(19) United States
(12) Patent Application Publication
(10) Pub. No.: US 2025/0129021 A1
(43) Pub. Date: Apr. 24, 2025

(54) SMALL MOLECULE PROTEIN SYNTHESIS MODULATORS

(71) Applicant: Interdict Bio, Inc., South San Francisco, CA (US)

(72) Inventors: David Gygi, San Francisco, CA (US); Sogole Sami Bahmanyar, San Francisco, CA (US); Lawrence Hamann, San Francisco, CA (US)

(73) Assignee: Interdict Bio, Inc., South San Francisco, CA (US)

(21) Appl. No.: 18/890,690

(22) Filed: Sep. 19, 2024

Related U.S. Application Data
(60) Provisional application No. 63/583,847, filed on Sep. 19, 2023.

(51) Int. Cl. C07D 207/12; A61K 31/40; A61K 31/4025; A61K 31/403; A61K 31/4035; A61K 31/407; A61K 31/41; A61K 31/4155; A61K 31/416; A61K 31/4178; A61K 31/4184; A61K 31/4192; A61K 31/4196; A61K 31/422; A61K 31/4245; A61K 31/427; A61K 31/428; A61K 31/429; A61K 31/433; A61K 31/437; A61K 31/4439; A61K 31/444; A61K 31/454; A61K 31/4725; A61K 31/496; A61K 31/499; A61K 31/501; A61K 31/506; A61K 31/519; A61K 31/5386; A61K 31/55; C07D 401/12; C07D 401/14; C07D 403/12; C07D 403/14; C07D 405/12; C07D 405/14; C07D 409/10; C07D 409/12; C07D 413/10; C07D 413/12; C07D 413/14; C07D 417/10; C07D 417/12; C07D 417/14; C07D 471/04; C07D 471/10; C07D 487/04; C07D 487/10; C07D 495/04; C07D 498/08; C07D 513/04; C07F 5/02

(52) U.S. Cl. CPC C07D 207/12; A61K 31/40; A61K 31/4025; A61K 31/403; A61K 31/4035; A61K 31/407; A61K 31/41; A61K 31/4155; A61K 31/416; A61K 31/4178; A61K 31/4184; A61K 31/4192; A61K 31/4196; A61K 31/422; A61K 31/4245; A61K 31/427; A61K 31/428; A61K 31/429; A61K 31/433; A61K 31/437; A61K 31/4439; A61K 31/444; A61K 31/454; A61K 31/4725; A61K 31/496; A61K 31/499; A61K 31/501; A61K 31/506; A61K 31/519; A61K 31/5386; A61K 31/55; C07D 401/12; C07D 401/14; C07D 403/12; C07D 403/14; C07D 405/12; C07D 405/14; C07D 409/10; C07D 409/12; C07D 413/10; C07D 413/12; C07D 413/14; C07D 417/10; C07D 417/12; C07D 417/14; C07D 471/04; C07D 471/10; C07D 487/04; C07D 487/10; C07D 495/04; C07D 498/08; C07D 513/04; C07F 5/025

(57) ABSTRACT

The present disclosure provides compounds of the formulae herein (e.g., Formula (I), Formula (V)), and pharmaceutically acceptable salts thereof, which are useful for modulating protein synthesis (e.g., modulating synthesis of BCL-2, MYC, CCND1, MCL-1, ALK, KRAS-G12D). The present disclosure also provides pharmaceutical compositions and kits comprising the compounds, or pharmaceutically acceptable salts thereof, and methods of treating or preventing diseases or disorders (e.g., diseases or disorders associated with BCL-2, MYC, CCND1, MCL-1, ALK, KRAS-G12D) by administering to a subject in need thereof the compounds, or pharmaceutically acceptable salts thereof, or pharmaceutical compositions thereof.

### CROSS-REFERENCE TO RELATED APPLICATIONS

This application claims the benefit of and priority under 35 U.S.C. § 119(e) to U.S. Provisional Application No. 63/583,847, filed Sep. 19, 2023, titled SMALL MOLECULE PROTEIN MODULATORS, the contents of which are incorporated herewith by reference in their entirety.

### BACKGROUND OF THE INVENTION

Small molecule therapeutics have for decades largely focused on binding to the target protein of interest to inhibit its action, or to induce activation of its function. In the case of inhibition, the net effect of the drug binding to its target is to sequester the target, making it unable to effectively perform its native (or in the case of certain pathologic states, aberrant) function. More recently, small molecule therapeutics have also been developed which alter protein homeostasis. Protein homeostasis refers to the equilibrium between protein synthesis and protein turnover, or degradation. In one instance, the existing pool of a given protein inside the cell may be diminished by accelerating the induction of its degradation. This can be achieved by small molecules that bind to a target and/or an E3 ligase, whereby the net result of binding is the induction of the target's degradation by native cellular machinery, as is the case for both molecular glues and PROteolysis TArgeting ChimeraS (PROTACS). The counterbalancing aspect of protein homeostasis relates to modulating the rate of protein synthesis. In this manner, one means to address specific protein targets is to block their synthesis by inhibition of the translation machinery of the cell. Protein synthesis takes place in the ribosome, where a molecule of mRNA encoding for the protein of interest is translated into the protein through a sequence of steps including initiation, elongation, and termination. Small molecules can bind either to elongation or initiation accessory factors to disable proper assembly and operation of the translation machinery or bind directly inside the ribosome to impede translation. In this way, cellular levels of a given protein can be downregulated to ameliorate diseases arising from an overabundance of pathologic proteins. This has broad applications in all therapeutic areas, including, but not limited to oncology, immunology and inflammation, neurodegeneration, cardiovascular and metabolic diseases, rare genetic diseases, and infectious diseases.

### SUMMARY OF THE INVENTION

The present invention relates to small molecules that modulate protein synthesis by inhibiting the translation machinery. These compounds demonstrate therapeutic utility, including but not limited to, in their ability to kill cancer cells.

In one aspect, the present disclosure provides compounds of Formula (I):

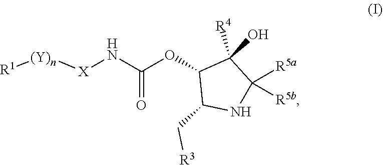

or pharmaceutically acceptable salts thereof, wherein X, R¹, R³, R⁴, R^{5a}, R^{5b}, Y, and n are as defined herein.

In another aspect, the present disclosure provides compounds of Formula (V):

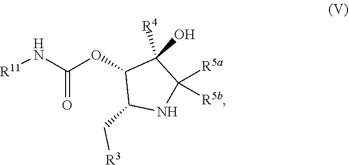

or pharmaceutically acceptable salts thereof, wherein R³, R⁴, R^{5a}, and R¹¹ are as defined herein.

In another aspect, the present disclosure provides pharmaceutical compositions comprising a compound disclosed herein. In some embodiments, the pharmaceutical composition comprises an excipient.

In another aspect, the present disclosure provides methods of modulating protein synthesis in a subject in need thereof or in a cell, tissue, or biological sample, comprising administering to the subject in need thereof or contacting the cell, tissue, or biological sample with an effective amount of a provided compound, or a pharmaceutical composition thereof.

In another aspect, the present disclosure provides methods of decreasing protein synthesis in a subject in need thereof or in a cell, tissue, or biological sample, comprising administering to the subject in need thereof or contacting the cell, tissue, or biological sample with an effective amount of a provided compound, or a pharmaceutical composition thereof.

In another aspect, the present disclosure provides methods of treating or preventing a disease or disorder in a subject in need thereof, comprising administering to the subject in need thereof a therapeutically effective amount of a provided compound, or a pharmaceutical composition thereof.

In another aspect, the present disclosure provides kits comprising a provided compound or pharmaceutical composition and instructions for its use.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The present disclosure additionally encompasses compounds as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C_{1-6_}alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylindenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

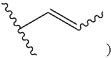

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylindenyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 □ electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 □ electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(Rb)₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{a}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{a})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rd groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg}groups;
   each instance of R^{f} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)₊X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxy,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “alkoxy” as used herein, refers to an alkyl group, as defined herein, appended to the parent molecular moiety through an oxygen atom. Representative examples of alkoxy include, but are not limited to, methoxy, ethoxy, n-propoxy, isopropoxy, n-butoxy and tert-butoxy.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{a} is as defined herein.

The term “boronyl” refers to boranes, boronic acids, boronic esters, borinic acids, and borinic esters, e.g., boronyl groups of the formula —B(R^{aa})₂, —B(OR^{cc})₂, and —BR^{aa}(OR^{cc}), wherein R^{aa} and R^{cc} are as defined herein.

The term “phosphino” refers to the group —P(R^{cc})₂, wherein R^{cc} is as defined herein.

The term “phosphono” refers to the group —(P═O)(OR^{cc})₂, wherein R^{aa} and R^{cc} are as defined herein.

The term “phosphoramido” refers to the group —O(P═O)(N(R^{bb})₂)₂, wherein each R^{bb} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyrrolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, 4,4′-dimethoxytrityl (4,4′-dimethoxytriphenylmethyl or DMT), a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent.

Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂—OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The present disclosure is not limited in any manner by the above exemplary listing of substituents.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the present disclosure is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the present disclosure are administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for modulating protein synthesis (e.g., decreasing protein synthesis). In certain embodiments, a therapeutically effective amount is an amount sufficient for treating a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis). In certain embodiments, a therapeutically effective amount is an amount sufficient for modulating protein synthesis (e.g., decreasing protein synthesis) and a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis).

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for modulating protein synthesis (e.g., decreasing protein synthesis). In certain embodiments, a prophylactically effective amount is an amount sufficient for treating a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis). In certain embodiments, a prophylactically effective amount is an amount sufficient for modulating protein synthesis (e.g., decreasing protein synthesis) and a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis).

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The term “about X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast, triple-negative breast cancer (TNBC)); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, hemolytic autoimmune anemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischemic colitis, diversion colitis, Behcet's syndrome, indeterminate colitis) and inflammatory bowel syndrome (IBS)), lupus, multiple sclerosis, morphea, myasthenia gravis, myocardial ischemia, nephrotic syndrome, pemphigus vulgaris, pernicious anemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, scleroderma, sarcoidosis, spondyloarthropathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatitis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), Huntington's disease, and Friedreich's ataxia. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; cerebellar ataxia; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

Immune disorders, such as auto-immune disorders, include, but are not limited to, arthritis (including rheumatoid arthritis, spondyloarthropathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

A “kinase” is a type of enzyme that transfers phosphate groups from high energy donor molecules, such as ATP, to specific substrates, referred to as phosphorylation. Kinases are part of the larger family of phosphotransferases. One of the largest groups of kinases are protein kinases, which act on and modify the activity of specific proteins. Kinases are used extensively to transmit signals and control complex processes in cells. Various other kinases act on small molecules such as lipids, carbohydrates, amino acids, and nucleotides, either for signaling or to prime them for metabolic pathways. Kinases are often named after their substrates. More than 500 different protein kinases have been identified in humans. These exemplary human protein kinases include, but are not limited to, AAK1, ABL, ACK, ACTR2, ACTR2B, AKT1, AKT2, AKT3, ALK, ALK1, ALK2, ALK4, ALK7, AMPKa1, AMPKa2, ANKRD3, ANPa, ANPb, ARAF, ARAFps, ARG, AurA, AurAps1, AurAps2, AurB, AurBps1, AurC, AXL, BARK1, BARK2, BIKE, BLK, BMPR1A, BMPR1Aps1, BMPR1Aps2, BMPR1B, BMPR2, BMX, BRAF, BRAFps, BRK, BRSK1, BRSK2, BTK, BUB1, BUBR1, CaMK1a, CaMK1b, CaMK1d, CaMK1g, CaMK2a, CaMK2b, CaMK2d, CaMK2g, CaMK4, CaMKK1, CaMKK2, caMLCK, CASK, CCK4, CCRK, CDC2, CDC7, CDK10, CDK11, CDK2, CDK3, CDK4, CDK4ps, CDK5, CDK5ps, CDK6, CDK7, CDK7ps, CDK8, CDK8ps, CDK9, CDKL1, CDKL2, CDKL3, CDKL4, CDKL5, CGDps, CHED, CHK1, CHK2, CHK2ps1, CHK2ps2, CK1a, CK1a2, CK1aps1, CK1aps2, CK1aps3, CK1d, CK1e, CK1g1, CK1g2, CK1g2ps, CK1g3, CK2a1, CK2a1-rs, CK2a2, CLIK1, CLIK1L, CLK1, CLK2, CLK2ps, CLK3, CLK3ps, CLK4, COT, CRIK, CRK7, CSK, CTK, CYGD, CYGF, DAPK1, DAPK2, DAPK3, DCAMKL1, DCAMKL2, DCAMKL3, DDR1, DDR2, DLK, DMPK1, DMPK2, DRAK1, DRAK2, DYRK1A, DYRK1B, DYRK2, DYRK3, DYRK4, EGFR, EphA1, EphA10, EphA2, EphA3, EphA4, EphA5, EphA6, EphA7, EphA8, EphB1, EphB2, EphB3, EphB4, EphB6, Erk1, Erk2, Erk3, Erk3ps1, Erk3ps2, Erk3ps3, Erk3ps4, Erk4, Erk5, Erk7, FAK, FER, FERps, FES, FGFR1, FGFR2, FGFR3, FGFR4, FGR, FLT1, FLT1ps, FLT3, FLT4, FMS, FRK, Fused, FYN, GAK, GCK, GCN2, GCN22, GPRK4, GPRK5, GPRK6, GPRK6ps, GPRK7, GSK3A, GSK3B, Haspin, HCK, HER2/ErbB2, HER3/ErbB3, HER4/ErbB4, HH498, HIPK1, HIPK2, HIPK3, HIPK4, HPK1, HRI, HRIps, HSER, HUNK, ICK, IGF1R, IKKa, IKKb, IKKe, ILK, INSR, IRAK1, IRAK2, IRAK3, IRAK4, IRE1, IRE2, IRR, ITK, JAKI, JAK2, JAK3, JNK1, JNK2, JNK3, KDR, KHS1, KHS2, KIS, KIT, KSGCps, KSR1, KSR2, LATS1, LATS2, LCK, LIMK1, LIMK2, LIMK2ps, LKB1, LMR1, LMR2, LMR3, LOK, LRRK1, LRRK2, LTK, LYN, LZK, MAK, MAP2K1, MAP2K1ps, MAP2K2, MAP2K2ps, MAP2K3, MAP2K4, MAP2K5, MAP2K6, MAP2K7, MAP3K1, MAP3K2, MAP3K3, MAP3K4, MAP3K5, MAP3K6, MAP3K7, MAP3K8, MAPKAPK2, MAPKAPK3, MAPKAPK5, MAPKAPKps1, MARK1, MARK2, MARK3, MARK4, MARKps01, MARKps02, MARKps03, MARKps04, MARKps05, MARKps07, MARKps08, MARKps09, MARKps10, MARKps11, MARKps12, MARKps13, MARKps15, MARKps16, MARKps17, MARKps18, MARKps19, MARKps20, MARKps21, MARKps22, MARKps23, MARKps24, MARKps25, MARKps26, MARKps27, MARKps28, MARKps29, MARKps30, MAST1, MAST2, MAST3, MAST4, MASTL, MELK, MER, MET, MISR2, MLK1, MLK2, MLK3, MLK4, MLKL, MNK1, MNK1ps, MNK2, MOK, MOS, MPSK1, MPSK1ps, MRCKa, MRCKb, MRCKps, MSK1, MSK12, MSK2, MSK22, MSSK1, MST1, MST2, MST3, MST3ps, MST4, MUSK, MYO3A, MYO3B, MYT1, NDR1, NDR2, NEK1, NEK10, NEK11, NEK2, NEK2ps1, NEK2ps2, NEK2ps3, NEK3, NEK4, NEK4ps, NEK5, NEK6, NEK7, NEK8, NEK9, NIK, NIM1, NLK, NRBP1, NRBP2, NuaK1, NuaK2, Obscn, Obscn2, OSR1, p38a, p38b, p38d, p38g, p70S6K, p70S6Kb, p70S6Kps1, p70S6Kps2, PAK1, PAK2, PAK2ps, PAK3, PAK4, PAK5, PAK6, PASK, PBK, PCTAIRE1, PCTAIRE2, PCTAIRE3, PDGFRa, PDGFRb, PDK1, PEK, PFTAIRE1, PFTAIRE2, PHKg1, PHKg1ps1, PHKg1ps2, PHKg1ps3, PHKg2, PIK3R4, PIM1, PIM2, PIM3, PINK1, PITSLRE, PKACa, PKACb, PKACg, PKCa, PKCb, PKCd, PKCe, PKCg, PKCh, PKCi, PKCips, PKCt, PKCz, PKD1, PKD2, PKD3, PKG1, PKG2, PKN1, PKN2, PKN3, PKR, PLK1, PLK1ps1, PLK1ps2, PLK2, PLK3, PLK4, PRKX, PRKXps, PRKY, PRP4, PRP4ps, PRPK, PSKH1, PSKH1ps, PSKH2, PYK2, QIK, QSK, RAF1, RAF1ps, RET, RHOK, RIPK1, RIPK2, RIPK3, RNAseL, ROCK1, ROCK2, RON, ROR1, ROR2, ROS, RSK1, RSK12, RSK2, RSK22, RSK3, RSK32, RSK4, RSK42, RSKL1, RSKL2, RYK, RYKps, SAKps, SBK, SCYL1, SCYL2, SCYL2ps, SCYL3, SGK, SgK050ps, SgK069, SgK071, SgK085, SgK110, SgK196, SGK2, SgK223, SgK269, SgK288, SGK3, SgK307, SgK384ps, SgK396, SgK424, SgK493, SgK494, SgK495, SgK496, SIK (e.g., SIK1, SIK2), skMLCK, SLK, Slob, smMLCK, SNRK, SPEG, SPEG2, SRC, SRM, SRPK1, SRPK2, SRPK2ps, SSTK, STK33, STK33ps, STLK3, STLK5, STLK6, STLK6ps1, STLK6-rs, SuRTK106, SYK, TAK1, TAO1, TAO2, TAO3, TBCK, TBK1, TEC, TESK1, TESK2, TGFbR1, TGFbR2, TIE1, TIE2, TLK1, TLK1ps, TLK2, TLK2ps1, TLK2ps2, TNK1, Trad, Trb1, Trb2, Trb3, Trio, TRKA, TRKB, TRKC, TSSK1, TSSK2, TSSK3, TSSK4, TSSKps1, TSSKps2, TTBK1, TTBK2, TTK, TTN, TXK, TYK2, TYK22, TYRO3, TYRO3ps, ULK1, ULK2, ULK3, ULK4, VACAMKL, VRK1, VRK2, VRK3, VRK3ps, Wee1, Wee1B, Wee1Bps, Wee1ps1, Wee1ps2, Wnk1, Wnk2, Wnk3, Wnk4, YANK1, YANK2, YANK3, YES, YESps, YSK1, ZAK, ZAP70, ZC1/HGK, ZC2/TNIK, ZC3/MINK, and ZC4/NRK.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “inhibition,” “inhibiting,” “inhibit,” or “inhibitor” refer to the ability of a compound to reduce, slow, halt or prevent activity of a particular biological process (e.g., protein activity, protein synthesis) in a cell relative to vehicle.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compounds

In one aspect, the present disclosure provides a compound of Formula (I):

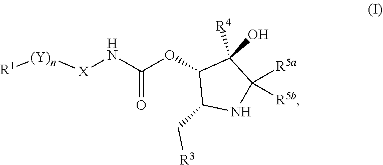

or a pharmaceutically acceptable salt thereof, wherein:
X is —CH(R²)— or —NH—;
R¹ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{1a}, or —N(R^{1a})₂;
each instance of R^{1a} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or two instances of R^{1a} are joined together with their intervening atom to form an optionally substituted heterocyclic ring or optionally substituted heteroaryl ring;
R² is hydrogen or optionally substituted alkyl;
or R¹ and R² are joined together with their intervening atoms to form optionally substituted carbocyclyl, or optionally substituted heterocyclyl;
R³ is optionally substituted carbocyclyl, optionally substituted heterocyclyl, optionally substituted aryl, or optionally substituted heteroaryl;
R⁴ is hydrogen or optionally substituted alkyl;
R^{5a} and R^{5b} are each hydrogen; or R^{5a} and R^{5b} are joined together with their intervening atom to form optionally substituted carbocyclyl;
each instance of Y is independently —C(R^{Y})₂—, —O—, or —N(R^{1a})—;
each instance of R^{Y} is independently hydrogen or halogen, or two instances of R^{Y} are taken together to form ═O; and
n is 0, 1, 2, 3, 4, or 5.

In another aspect, the present disclosure provides a compound of Formula (V):

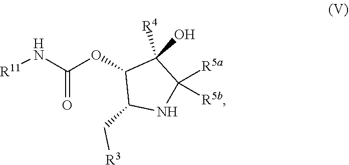

or a pharmaceutically acceptable salt thereof, wherein:
R³ is optionally substituted carbocyclyl, optionally substituted heterocyclyl, optionally substituted aryl, or optionally substituted heteroaryl;
R⁴ is hydrogen or optionally substituted alkyl;
R^{5a} and R^{5b} are each hydrogen; or R^{5a} and R^{5b} are joined together with their intervening atom to form optionally substituted carbocyclyl; and
R¹¹ is optionally substituted aryl or optionally substituted heteroaryl.
   R¹, R², X, Y, R^{1a}, R^{Y}, and n

As generally described herein, R¹ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{1a}, or —N(R^{1a})₂; and R² is hydrogen or optionally substituted alkyl; or R¹ and R² are joined together with their intervening atoms to form optionally substituted carbocyclyl or optionally substituted heterocyclyl.

In some embodiments, R¹ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{1a}, or —N(R^{1a})₂. In some embodiments, R¹ is hydrogen, optionally substituted alkyl, optionally substituted carbocyclyl, optionally substituted heterocyclyl, optionally substituted aryl, optionally substituted heteroaryl, —OR^{1a}, or —N(R^{1a})₂. In some embodiments, R¹ is hydrogen, optionally substituted alkyl, optionally substituted alkenyl, optionally substituted alkynyl, —OR^{1a}, or —N(R^{1a})₂. In some embodiments, R¹ is hydrogen, optionally substituted alkyl, optionally substituted alkenyl, or optionally substituted alkynyl. In some embodiments, R¹ is hydrogen, optionally substituted alkyl, —OR^{1a}, or —N(R^{1a})₂. In some embodiments, R¹ is hydrogen or optionally substituted alkyl. In some embodiments, R¹ is hydrogen, optionally substituted alkyl, optionally substituted carbocyclyl, optionally substituted heterocyclyl, optionally substituted aryl, optionally substituted heteroaryl, —OR^{1a}, or —N(R^{1a})₂. In some embodiments, R¹ is hydrogen, optionally substituted alkyl, optionally substituted carbocyclyl, optionally substituted heterocyclyl, optionally substituted aryl, or optionally substituted heteroaryl. In some embodiments, R¹ is optionally substituted alkyl, optionally substituted carbocyclyl, optionally substituted heterocyclyl, optionally substituted aryl, optionally substituted heteroaryl, —OR^{1a}, or —N(R^{1a})₂. In some embodiments, R¹ is optionally substituted alkyl, optionally substituted carbocyclyl, optionally substituted heterocyclyl, optionally substituted aryl, or optionally substituted heteroaryl. In some embodiments, R¹ is optionally substituted carbocyclyl, optionally substituted heterocyclyl, optionally substituted aryl, or optionally substituted heteroaryl. In some embodiments, R¹ is optionally substituted carbocyclyl or optionally substituted heterocyclyl. In some embodiments, R¹ is optionally substituted aryl or optionally substituted heteroaryl. In some embodiments, R¹ is optionally substituted heterocyclyl, optionally substituted aryl, or optionally substituted heteroaryl. In some embodiments, R¹ is —OR^{1a} or —N(R^{1a})₂.

In some embodiments, R¹ is hydrogen, optionally substituted alkyl, optionally substituted alkenyl, or optionally substituted alkynyl. In some embodiments, R¹ is hydrogen. In some embodiments, R¹ is optionally substituted alkyl. In some embodiments, R¹ is optionally substituted C₁₋₁₂ alkyl. In some embodiments, R¹ is optionally substituted C₁₋₈ alkyl. In some embodiments, R¹ is optionally substituted C₈ alkyl. In some embodiments, R¹ is optionally substituted C₇ alkyl. In some embodiments, R¹ is optionally substituted C₁₋₆ alkyl. In some embodiments, R¹ is optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In some embodiments, R¹ is substituted C₁₋₆ alkyl. In some embodiments, R¹ is substituted methyl, substituted ethyl, substituted n-propyl, substituted isopropyl, substituted n-butyl, substituted tert-butyl, substituted sec-butyl, substituted isobutyl, substituted n-pentyl, substituted 3-pentanyl, substituted amyl, substituted neopentyl, substituted 3-methyl-2-butanyl, substituted tert-amyl, or substituted n-hexyl. In some embodiments, R¹ is unsubstituted C₁₋₆ alkyl. In some embodiments, R¹ is methyl, ethyl, n-propyl, isopropyl, n-butyl, tert-butyl, sec-butyl, isobutyl, n-pentyl, 3-pentanyl, amyl, neopentyl, 3-methyl-2-butanyl, tert-amyl, or n-hexyl. In some embodiments, R¹ is methyl or isopropyl. In some embodiments, R¹ is optionally substituted methyl. In some embodiments, R¹ is substituted methyl. In some embodiments, R¹ is methyl. In some embodiments, R¹ is optionally substituted ethyl. In some embodiments, R¹ is substituted ethyl. In some embodiments, R¹ is ethyl. In some embodiments, R¹ is optionally substituted isopropyl. In some embodiments, R¹ is substituted isopropyl. In some embodiments, R¹ is isopropyl.

In some embodiments, R¹ is alkyl substituted with ═O, ═S, optionally substituted aryl, optionally substituted heteroaryl, —OR^{1a}, —N(R^{1a})₂, and/or halogen. In some embodiments, R¹ is C₁₋₆ alkyl substituted with ═O, ═S, optionally substituted aryl, optionally substituted heteroaryl, —OR^{1a}, —N(R^{1a})₂, and/or halogen. In some embodiments, R¹ is alkyl substituted with ═O, ═S, optionally substituted aryl, optionally substituted heteroaryl, and/or —N(R^{1a})₂. In some embodiments, R¹ is C₁₋₆ alkyl substituted with ═O, ═S, optionally substituted aryl, optionally substituted heteroaryl, and/or —N(R^{1a})₂.

In some embodiments, R¹ is alkyl substituted with ═O or ═S. In some embodiments, R¹ is alkyl substituted with ═O. In some embodiments, R¹ is C₁₋₆ alkyl substituted with ═O. In some embodiments, R¹ is optionally substituted methyl substituted with ═O. In some embodiments, R¹ is optionally substituted ethyl substituted with ═O. In some embodiments, R¹ is optionally substituted n-propyl substituted with ═O.

In some embodiments, R¹ is alkyl substituted with optionally substituted aryl or optionally substituted heteroaryl. In some embodiments, R¹ is alkyl substituted with optionally substituted aryl. In some embodiments, R¹ is alkyl substituted with optionally substituted phenyl. In some embodiments, R¹ is alkyl substituted with phenyl. In some embodiments, R¹ is C₁₋₆ alkyl substituted with optionally substituted aryl or optionally substituted heteroaryl. In some embodiments, R¹ is C₁₋₆ alkyl substituted with optionally substituted phenyl. In some embodiments, R¹ is C₁₋₆ alkyl substituted with phenyl. In some embodiments, R¹ is methyl substituted with phenyl. In some embodiments, R¹ is ethyl substituted with phenyl. In some embodiments, R¹ is propyl substituted with phenyl. In some embodiments, R¹ is alkyl substituted with optionally substituted heteroaryl.

In some embodiments, R¹ is alkyl substituted with —N(R^{1a})₂. In some embodiments, R¹ is C₁₋₆ alkyl substituted with —N(R^{1a})₂. In some embodiments, R¹ is C₁₋₆ alkyl substituted with —NH₂. In some embodiments, R¹ is methyl substituted with —N(R^{1a})₂. In some embodiments, R¹ is methyl substituted with —NH₂. In some embodiments, R¹ is ethyl substituted with —N(R^{1a})₂. In some embodiments, R¹ is isopropyl substituted with —N(R^{1a})₂.

In some embodiments, R¹ is alkyl substituted with —OR^{1a}. In some embodiments, R¹ is C₁₋₆ alkyl substituted with —OR^{1a}. In some embodiments, R¹ is alkyl substituted with halogen. In some embodiments, R¹ is alkyl substituted with —F. In some embodiments, R¹ is C₁₋₆ alkyl substituted with halogen. In some embodiments, R¹ is C₁₋₆ alkyl substituted with —F.

In some embodiments, R¹ is optionally substituted alkenyl. In some embodiments, R¹ is optionally substituted C₂₋₁₂ alkenyl. In some embodiments, R¹ is optionally substituted C₂₋₆ alkenyl. In some embodiments, R¹ is substituted C₂₋₆ alkenyl. In some embodiments, R¹ is unsubstituted C₂₋₆ alkenyl. In some embodiments, R¹ is optionally substituted ethenyl, optionally substituted 1-propenyl, optionally substituted 2-propenyl, optionally substituted 1-butenyl, optionally substituted 2-butenyl, optionally substituted pentenyl, or optionally substituted hexenyl. In some embodiments, R¹ is substituted ethenyl, substituted 1-propenyl, substituted 2-propenyl, substituted 1-butenyl, substituted 2-butenyl, substituted pentenyl, or substituted hexenyl. In some embodiments, R¹ is unsubstituted ethenyl, unsubstituted 1-propenyl, unsubstituted 2-propenyl, unsubstituted 1-butenyl, unsubstituted 2-butenyl, unsubstituted pentenyl, or unsubstituted hexenyl.

In some embodiments, R¹ is optionally substituted alkynyl. In some embodiments, R¹ is optionally substituted C₂₋₁₂ alkynyl. In some embodiments, R¹ is optionally substituted C₂₋₆ alkynyl. In some embodiments, R¹ is substituted C₂₋₆ alkynyl. In some embodiments, R¹ is unsubstituted C₂₋₆ alkynyl. In some embodiments, R¹ is optionally substituted ethynyl, optionally substituted 1-propynyl, optionally substituted 2-propynyl, optionally substituted 1-butynyl, optionally substituted 2-butynyl, optionally substituted pentynyl, or optionally substituted hexynyl. In some embodiments, R¹ is substituted ethynyl, substituted 1-propynyl, substituted 2-propynyl, substituted 1-butynyl, substituted 2-butynyl, substituted pentynyl, or substituted hexynyl. In some embodiments, R¹ is unsubstituted ethynyl, unsubstituted 1-propynyl, unsubstituted 2-propynyl, unsubstituted 1-butynyl, unsubstituted 2-butynyl, unsubstituted pentynyl, or unsubstituted hexynyl. In some embodiments, R¹ is optionally substituted ethynyl. In some embodiments, R¹ is substituted ethynyl. In some embodiments, R¹ is ethynyl.

In some embodiments, R¹ is hydrogen, methyl, ethyl, isopropyl,

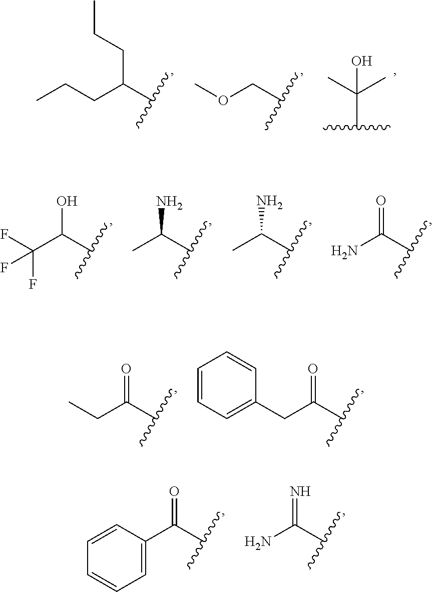

—CHF₂, —CF₃, —CH₂OCF₃, —CH₂SCF₃,

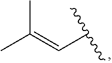 [SMILES: *C=C(C)C]

ethynyl, or

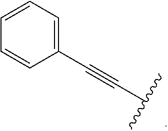 [SMILES: *C#Cc1ccccc1]

In some embodiments, R¹ is hydrogen, methyl, ethyl, isopropyl,

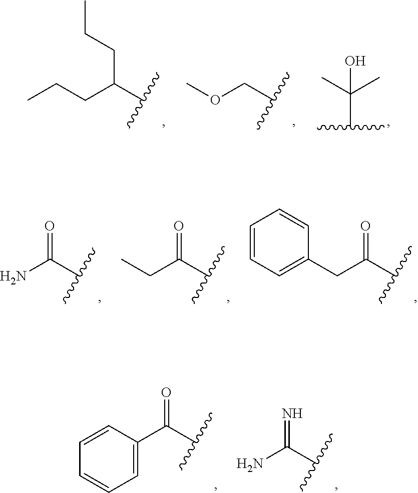

—CF₃, or ethynyl.

In some embodiments, R¹ is hydrogen, methyl, isopropyl,

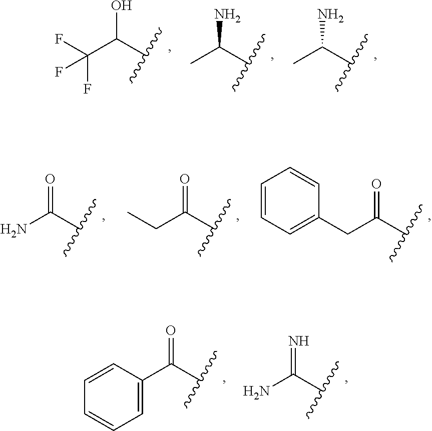

—CHF₂, —CF₃, —CH₂OCF₃, —CH₂SCF₃,

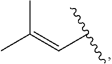 [SMILES: *C=C(C)C]

ethynyl, or

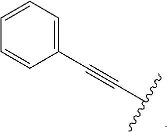 [SMILES: *C#Cc1ccccc1]

In some embodiments, R¹ is hydrogen, methyl, isopropyl,

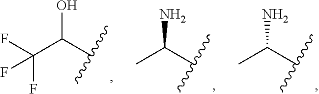

—CHF₂, —CH₂OCF₃, —CH₂SCF₃,

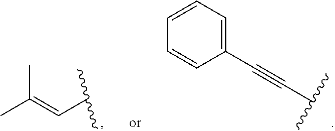

In some embodiments, R¹ is hydrogen, methyl, isopropyl,

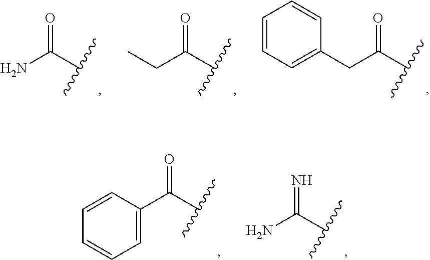

or ethynyl. In some embodiments, R¹ is methyl, isopropyl,

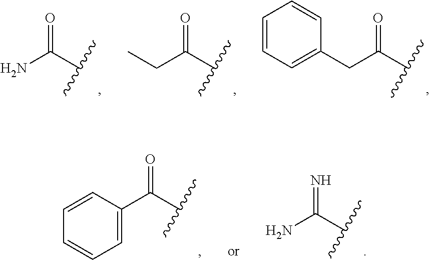

In some embodiments, R¹ is methyl or isopropyl. In some embodiments, R¹ is

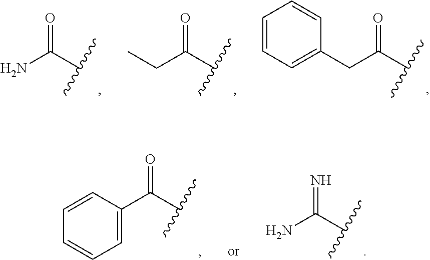

In some embodiments, R¹ is

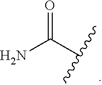 [SMILES: *C(N)=O]

In some embodiments, R¹ is

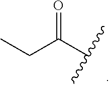 [SMILES: *C(=O)CC]

In some embodiments, R¹ is

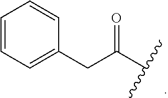 [SMILES: *C(=O)Cc1ccccc1]

In some embodiments, R¹ is

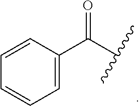 [SMILES: *C(=O)c1ccccc1]

In some embodiments, R¹ is

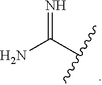 [SMILES: *C(=N)N]

In some embodiments, R¹ is optionally substituted carbocyclyl, optionally substituted heterocyclyl, optionally substituted aryl, or optionally substituted heteroaryl. In some embodiments, R¹ is optionally substituted carbocyclyl or optionally substituted heterocyclyl. In some embodiments, R¹ is optionally substituted carbocyclyl. In some embodiments, R¹ is optionally substituted C₃₋₁₀carbocyclyl. In some embodiments, R¹ is optionally substituted C₃₋₆ carbocyclyl. In some embodiments, R¹ is optionally substituted cyclopropyl, optionally substituted cyclobutyl, optionally substituted cyclobutenyl, optionally substituted cyclopentyl, or optionally substituted cyclohexyl. In some embodiments, R¹ is optionally substituted cyclopropyl or optionally substituted cyclobutenyl. In some embodiments, R¹ is substituted cyclopropyl or substituted cyclobutenyl. In some embodiments, R¹ is substituted cyclopropyl. In some embodiments, R¹ is substituted cyclobutenyl. In some embodiments, R¹ is unsubstituted cyclopropyl or unsubstituted cyclobutenyl. In some embodiments, R¹ is unsubstituted cyclopropyl. In some embodiments, R¹ is unsubstituted cyclobutenyl.

In some embodiments, R¹ is C₃₋₆ carbocyclyl substituted with halogen, —N(R^{1a})₂, and/or ═O. In some embodiments, R¹ is C₃₋₆ carbocyclyl substituted with halogen. In some embodiments, R¹ is C₃₋₆ carbocyclyl substituted with —F. In some embodiments, R¹ is

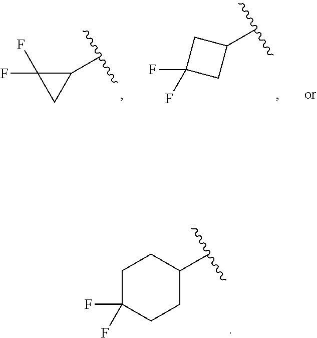 [SMILES: *C1CC1(F)F ; *C1CC(F)(F)C1 ; *C1CCC(F)(F)CC1]

In some embodiments, R¹ is C₃₋₆ carbocyclyl substituted with —N(R^{1a})₂. In some embodiments, R¹ is C₃₋₆ carbocyclyl substituted with —NH₂. In some embodiments, R¹ is C₃₋₆ carbocyclyl substituted with —NHR^{1a}. In some embodiments, R¹ is C₃₋₆ carbocyclyl substituted with —NHR^{1a}, and R^{1a} is optionally substituted alkyl. In some embodiments, R¹ is C₃₋₆ carbocyclyl substituted with —NHR^{1a}, and R^{1a} is benzyl. In some embodiments, R¹ is cyclopropyl substituted with —N(R^{1a})₂. In some embodiments, R¹ is cyclopropyl substituted with —NH₂. In some embodiments, R¹ is cyclobutenyl substituted with —N(R^{1a})₂. In some embodiments, R¹ is cyclobutenyl substituted with —NH₂. In some embodiments, R¹ is cyclobutenyl substituted with —NHR^{1a}. In some embodiments, R¹ is cyclobutenyl substituted with —NHR^{1a}, and R^{1a} is optionally substituted alkyl. In some embodiments, R¹ is cyclobutenyl substituted with —NHR^{1a}, and R^{1a} is benzyl or ethyl. In some embodiments, R¹ is C₃₋₆ carbocyclyl substituted with ═O. In some embodiments, R¹ is C₃₋₆ carbocyclyl substituted with —N(R^{1a})₂ and ═O. In some embodiments, R¹ is cyclobutenyl substituted with ═O. In some embodiments, R¹ is cyclobutenyl substituted with —N(R^{1a})₂ and ═O. In some embodiments, R¹ is

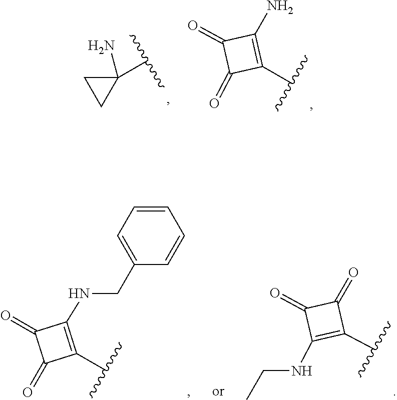

In some embodiments, R¹ is

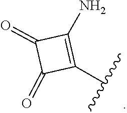 [SMILES: *c1c(N)c(=O)c1=O]

In some embodiments, R¹ is

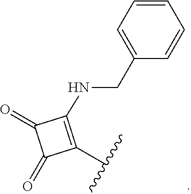 [SMILES: *c1c(NCc2ccccc2)c(=O)c1=O]

In some embodiments, R¹ is

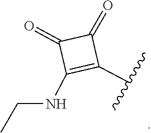 [SMILES: *c1c(NCC)c(=O)c1=O]

In some embodiments, R¹ is optionally substituted cyclopropyl, optionally substituted cyclobutyl, optionally substituted cyclohexyl, or optionally substituted cyclobutenyl. In some embodiments, R¹ is optionally substituted cyclopropyl or optionally substituted cyclobutenyl. In some embodiments, R¹ is

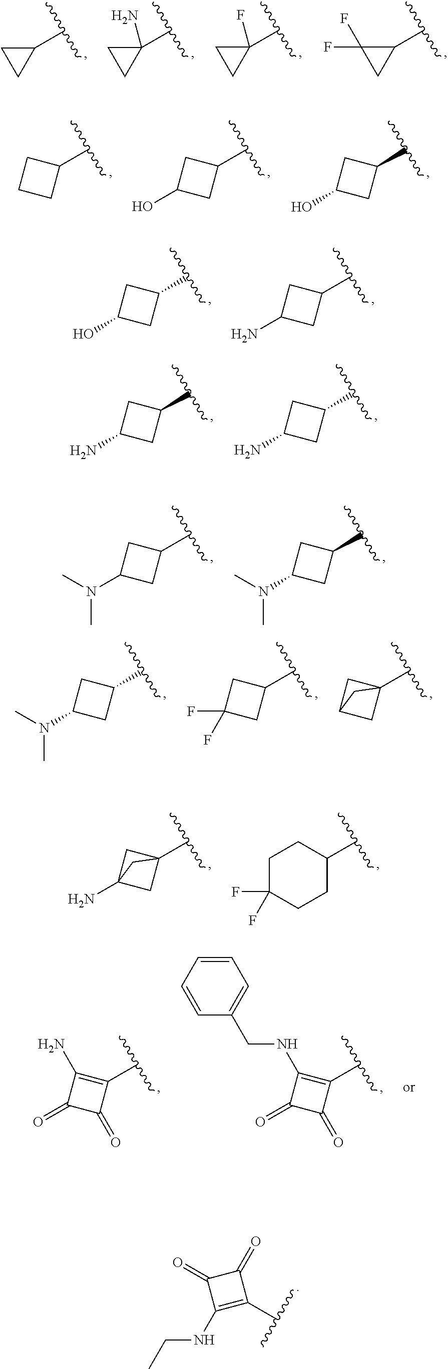

In some embodiments, R¹ is

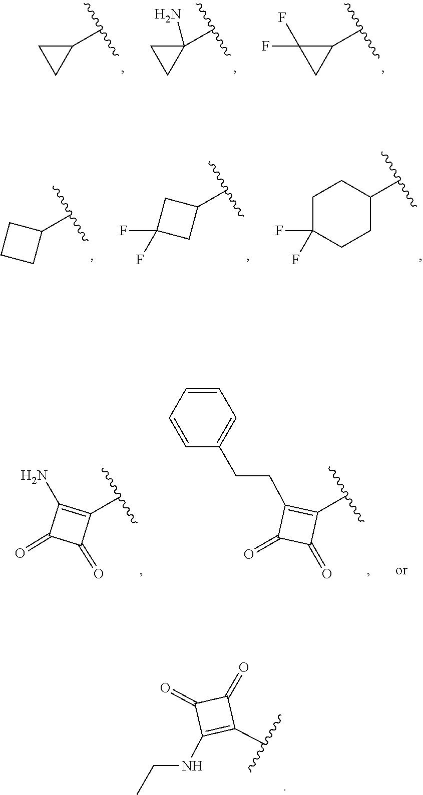

In some embodiments, R¹ is

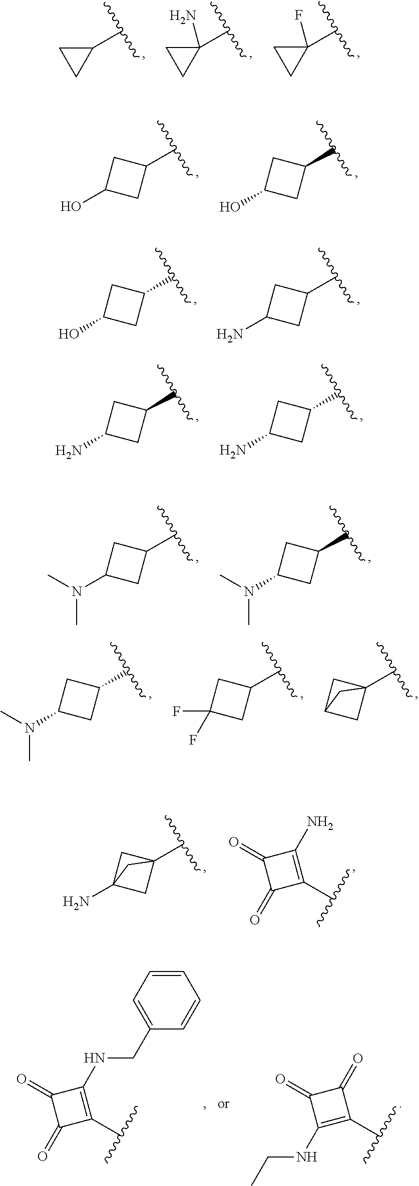

In some embodiments, R¹ is

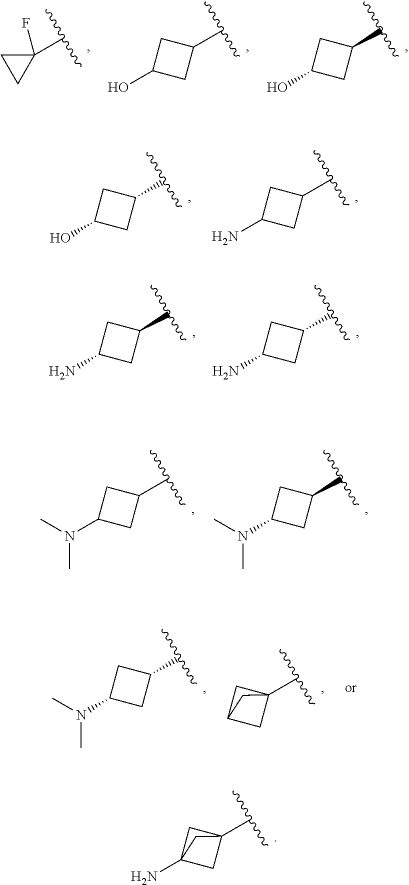

In some embodiments, R¹ is

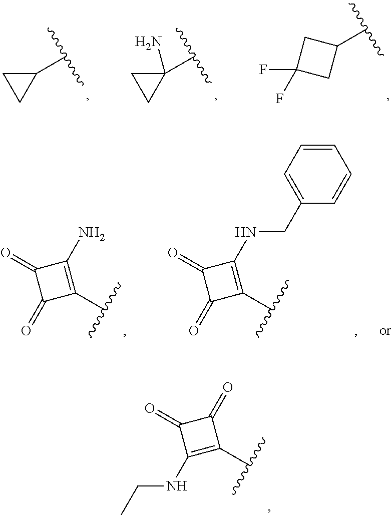

In some embodiments, R¹ is

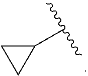 [SMILES: *C1CC1]

In some embodiments, R¹ is

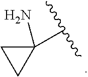 [SMILES: *C1(N)CC1]

In some embodiments, R¹ is

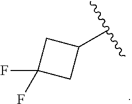 [SMILES: *C1CC(F)(F)C1]

In some embodiments, R¹ is

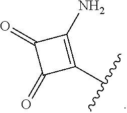 [SMILES: *c1c(N)c(=O)c1=O]

In some embodiments, R¹ is

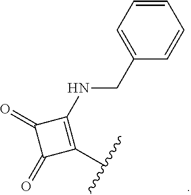 [SMILES: *c1c(NCc2ccccc2)c(=O)c1=O]

In some embodiments, R¹ is

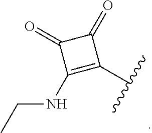 [SMILES: *c1c(NCC)c(=O)c1=O]

In some embodiments, R¹ is optionally substituted heterocyclyl, optionally substituted aryl, or optionally substituted heteroaryl. In some embodiments, R¹ is optionally substituted heterocyclyl. In some embodiments, R¹ is optionally substituted 3-14 membered heterocyclyl. In some embodiments, R¹ is optionally substituted tetrahydrofuranyl, optionally substituted tetrahydropyranyl, optionally substituted azetidinyl, optionally substituted pyrrolidinyl, optionally substituted imidazolidinyl, optionally substituted piperidinyl, optionally substituted piperazinyl, optionally substituted tetrahydroimidazopyrazinyl, optionally substituted tetrahydrothiopyranyl, optionally substituted 2,6-diazaspiro[3.3]heptyl, optionally substituted 6-azaspiro[3.4]octyl, 2,6-diazaspiro[3.4]octyl, optionally substituted 2,5,8-triazaspiro[3.5]nonyl, or optionally substituted 1,4,9-triazaspiro[5.5]undecyl. In some embodiments, R¹ is substituted tetrahydrofuranyl, substituted tetrahydropyranyl, substituted azetidinyl, substituted pyrrolidinyl, substituted imidazolidinyl, substituted piperidinyl, substituted piperazinyl, substituted tetrahydroimidazopyrazinyl, substituted tetrahydrothiopyranyl, substituted 2,6-diazaspiro[3.3]heptyl, substituted 6-azaspiro[3.4]octyl, 2,6-diazaspiro[3.4]octyl, substituted 2,5,8-triazaspiro[3.5]nonyl, or substituted 1,4,9-triazaspiro[5.5]undecyl. In some embodiments, R¹ is unsubstituted tetrahydrofuranyl, unsubstituted tetrahydropyranyl, unsubstituted azetidinyl, unsubstituted pyrrolidinyl, unsubstituted imidazolidinyl, unsubstituted piperidinyl, unsubstituted piperazinyl, unsubstituted tetrahydroimidazopyrazinyl, unsubstituted tetrahydrothiopyranyl, unsubstituted 2,6-diazaspiro[3.3]heptyl, unsubstituted 6-azaspiro[3.4]octyl, 2,6-diazaspiro[3.4]octyl, unsubstituted 2,5,8-triazaspiro[3.5]nonyl, or unsubstituted 1,4,9-triazaspiro[5.5]undecyl.

In some embodiments, R¹ is optionally substituted tetrahydrofuranyl, optionally substituted azetidinyl, optionally substituted pyrrolidinyl, optionally substituted imidazolidinyl, optionally substituted piperidinyl, optionally substituted piperazinyl, optionally substituted tetrahydrothiopyranyl, optionally substituted 2,6-diazaspiro[3.3]heptyl, optionally substituted 6-azaspiro[3.4]octyl, optionally substituted 2,6-diazaspiro[3.4]octyl, or optionally substituted 1,4,9-triazaspiro[5.5]undecyl. In some embodiments, R¹ is substituted tetrahydrofuranyl, substituted azetidinyl, substituted pyrrolidinyl, substituted imidazolidinyl, substituted piperidinyl, substituted piperazinyl, substituted tetrahydrothiopyranyl, substituted 2,6-diazaspiro[3.3]heptyl, substituted 6-azaspiro[3.4]octyl, substituted 2,6-diazaspiro[3.4]octyl, or substituted 1,4,9-triazaspiro[5.5]undecyl. In some embodiments, R¹ is unsubstituted tetrahydrofuranyl, unsubstituted azetidinyl, unsubstituted pyrrolidinyl, unsubstituted imidazolidinyl, unsubstituted piperidinyl, unsubstituted piperazinyl, unsubstituted tetrahydrothiopyranyl, unsubstituted 2,6-diazaspiro[3.3]heptyl, unsubstituted 6-azaspiro[3.4]octyl, unsubstituted 2,6-diazaspiro[3.4]octyl, or unsubstituted 1,4,9-triazaspiro[5.5]undecyl. In some embodiments, R¹ is optionally substituted tetrahydrofuranyl. In some embodiments, R¹ is optionally substituted azetidinyl. In some embodiments, R¹ is optionally substituted pyrrolidinyl. In some embodiments, R¹ is optionally substituted imidazolidinyl. In some embodiments, R¹ is optionally substituted piperidinyl. In some embodiments, R¹ is optionally substituted piperazinyl. In some embodiments, R¹ is optionally substituted tetrahydroimidazopyrazinyl. In some embodiments, R¹ is optionally substituted tetrahydrothiopyranyl. In some embodiments, R¹ is optionally substituted 2,6-diazaspiro[3.3]heptyl. In some embodiments, R¹ is optionally substituted 6-azaspiro[3.4]octyl. In some embodiments, R¹ is optionally substituted 2,6-diazaspiro[3.4]octyl. In some embodiments, R¹ is optionally substituted 2,5,8-triazaspiro[3.5]nonyl. In some embodiments, R¹ is optionally substituted 1,4,9-triazaspiro[5.5]undecyl.

In some embodiments, R¹ is optionally substituted 4-6 membered heterocyclyl. In some embodiments, R¹ is optionally substituted tetrahydrofuranyl, optionally substituted azetidinyl, optionally substituted pyrrolidinyl, optionally substituted imidazolidinyl, optionally substituted piperidinyl, optionally substituted piperazinyl, or optionally substituted tetrahydrothiopyranyl. In some embodiments, R¹ is optionally substituted azetidinyl, optionally substituted pyrrolidinyl, optionally substituted imidazolidinyl, optionally substituted piperidinyl, or optionally substituted piperazinyl.

In some embodiments, R¹ is 3-14 membered heterocyclyl substituted with ═O, optionally substituted alkyl, halogen, —OR^{1a}, —N(R^{1a})₂, and/or —CN. In some embodiments, R¹ is 3-14 membered heterocyclyl substituted with ═O. In some embodiments, R¹ is 3-14 membered heterocyclyl substituted with optionally substituted alkyl. In some embodiments, R¹ is 3-14 membered heterocyclyl substituted with optionally substituted C₁₋₆ alkyl. In some embodiments, R¹ is 3-14 membered heterocyclyl substituted with methyl. In some embodiments, R¹ is 3-14 membered heterocyclyl substituted with ═O and optionally substituted alkyl. In some embodiments, R¹ is 3-14 membered heterocyclyl substituted with ═O and methyl. In some embodiments, R¹ is 3-14 membered heterocyclyl substituted with halogen. In some embodiments, R¹ is 3-14 membered heterocyclyl substituted with —F. In some embodiments, R¹ is 3-14 membered heterocyclyl substituted with —OR^{1a}. In some embodiments, R¹ is 3-14 membered heterocyclyl substituted with —OH. In some embodiments, R¹ is 3-14 membered heterocyclyl substituted with —N(R^{1a})₂. In some embodiments, R¹ is 3-14 membered heterocyclyl substituted with —CN.

In some embodiments, R¹ is

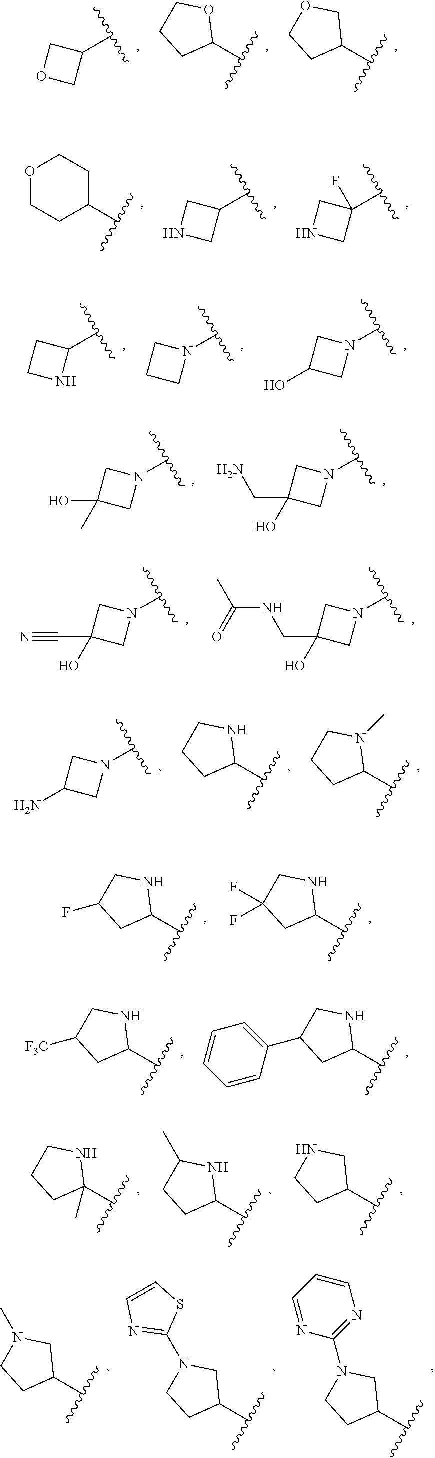

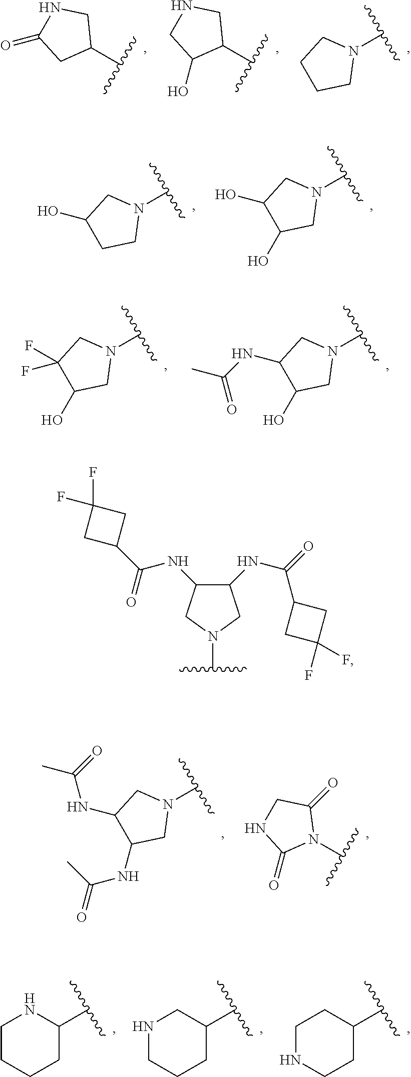

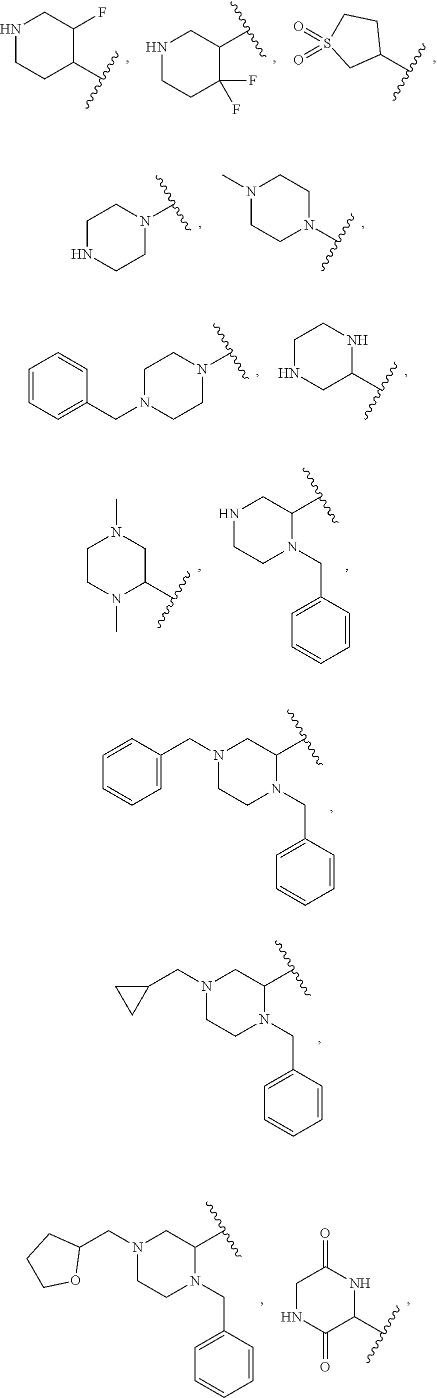

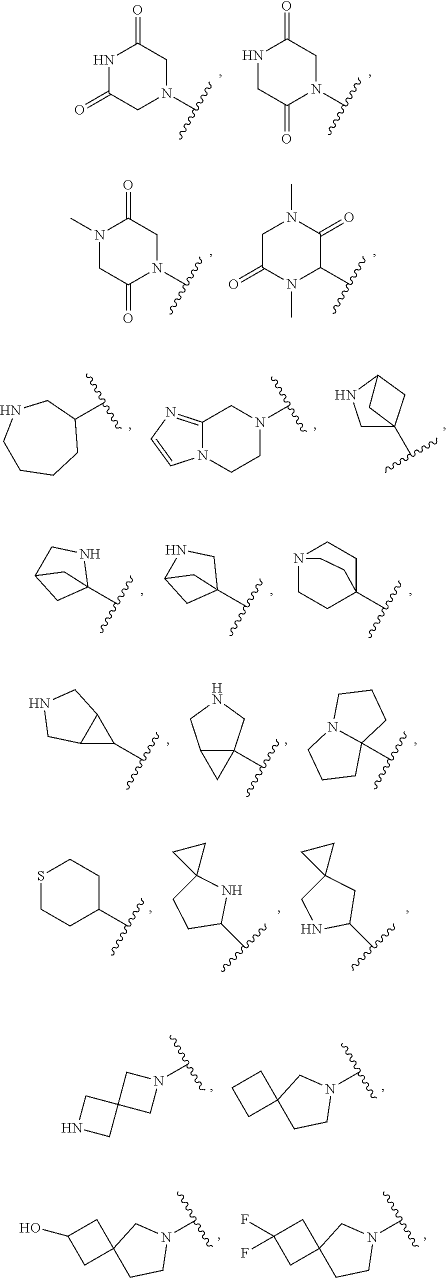

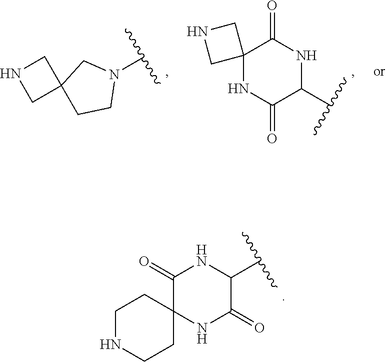 [SMILES: *N1CCC2(CNC2)C1 ; *C1NC(=O)C2(CNC2)NC1=O ; *C1NC(=O)C2(CCNCC2)NC1=O]

In some embodiments, R¹ is

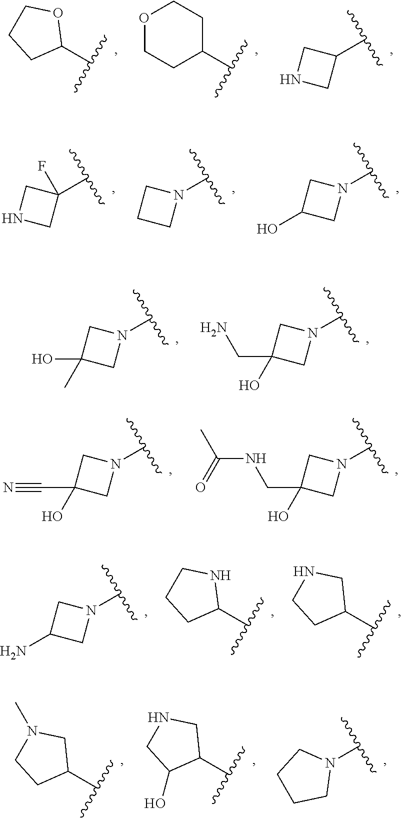

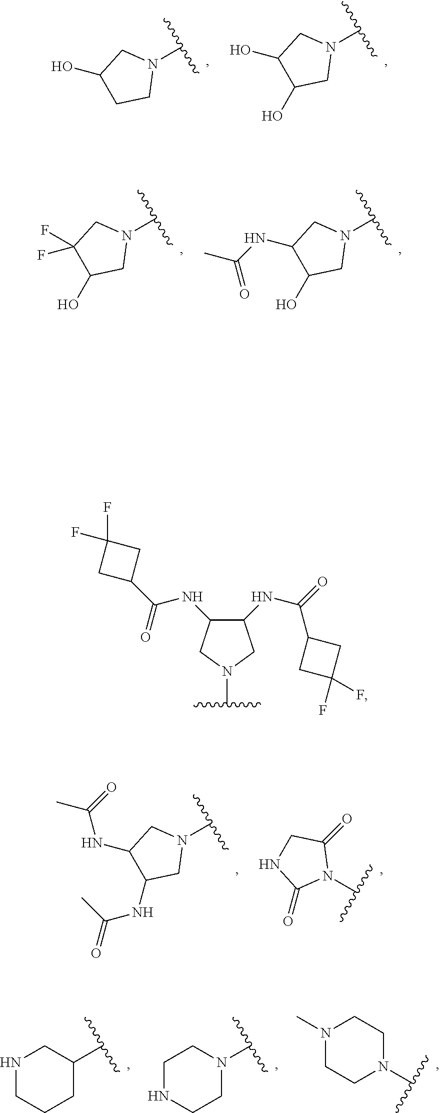

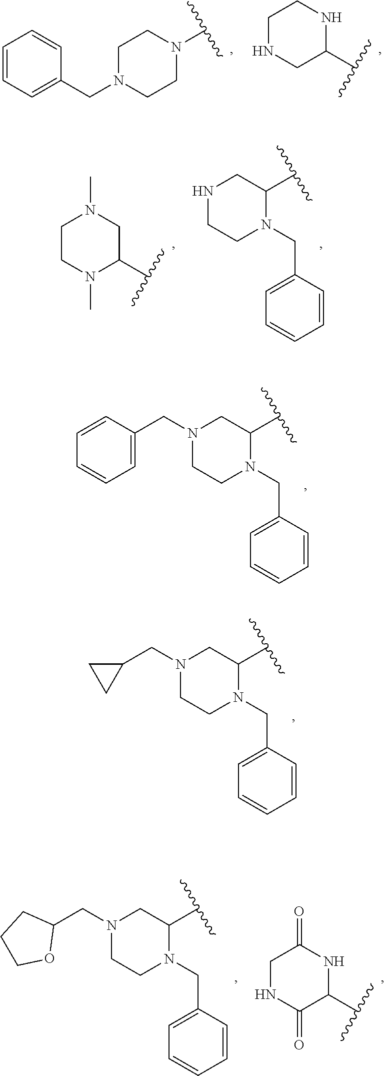

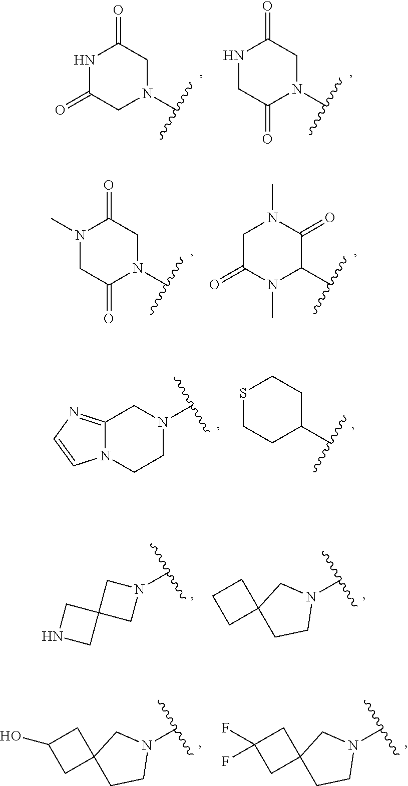

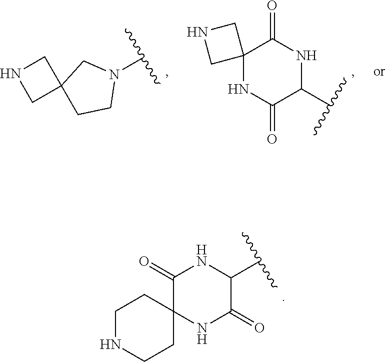 [SMILES: *N1CCC2(CNC2)C1 ; *C1NC(=O)C2(CNC2)NC1=O ; *C1NC(=O)C2(CCNCC2)NC1=O]

In some embodiments, R¹ is

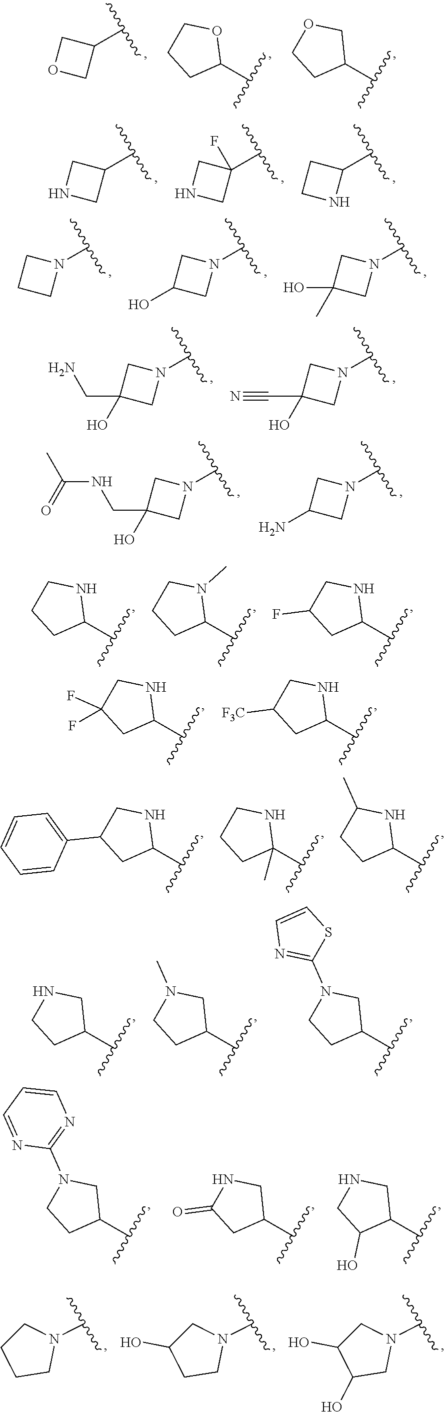

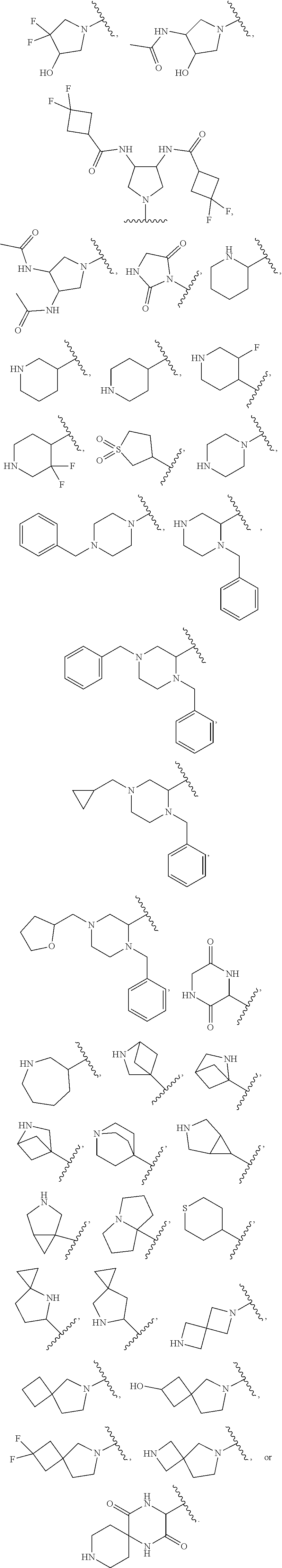

In some embodiments, R¹ is

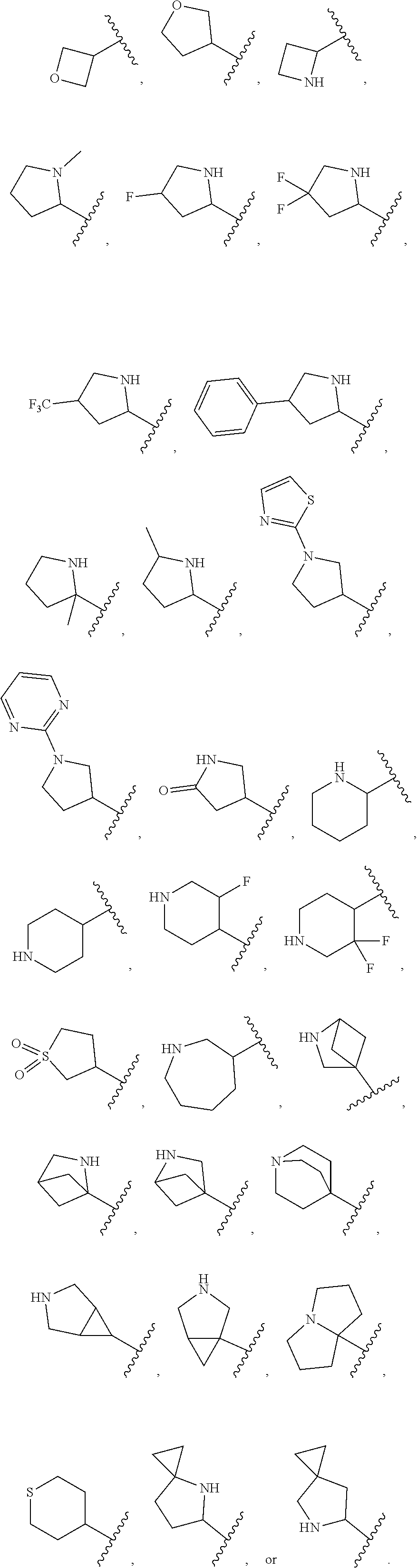

In some embodiments, R¹ is

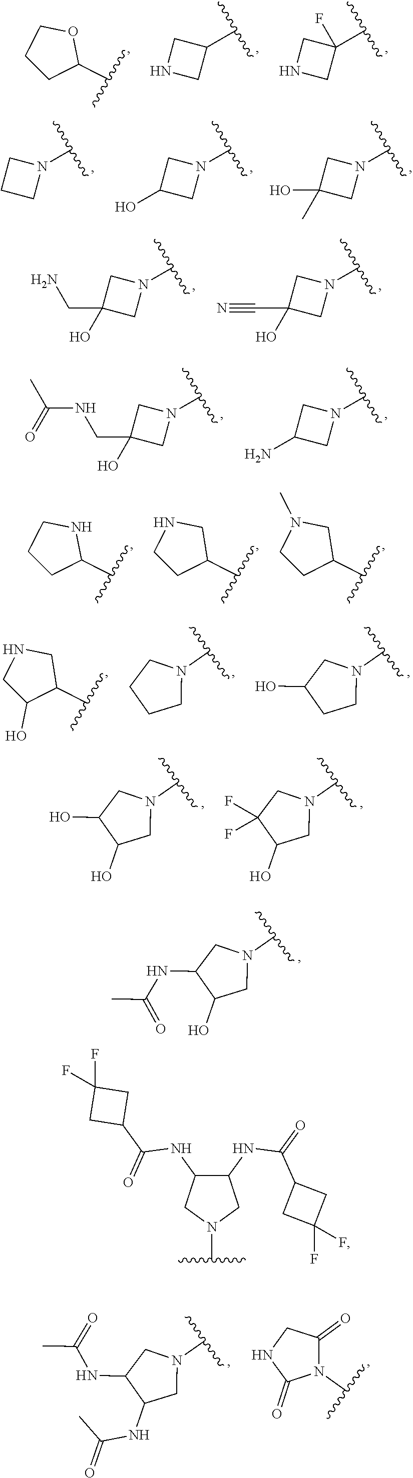

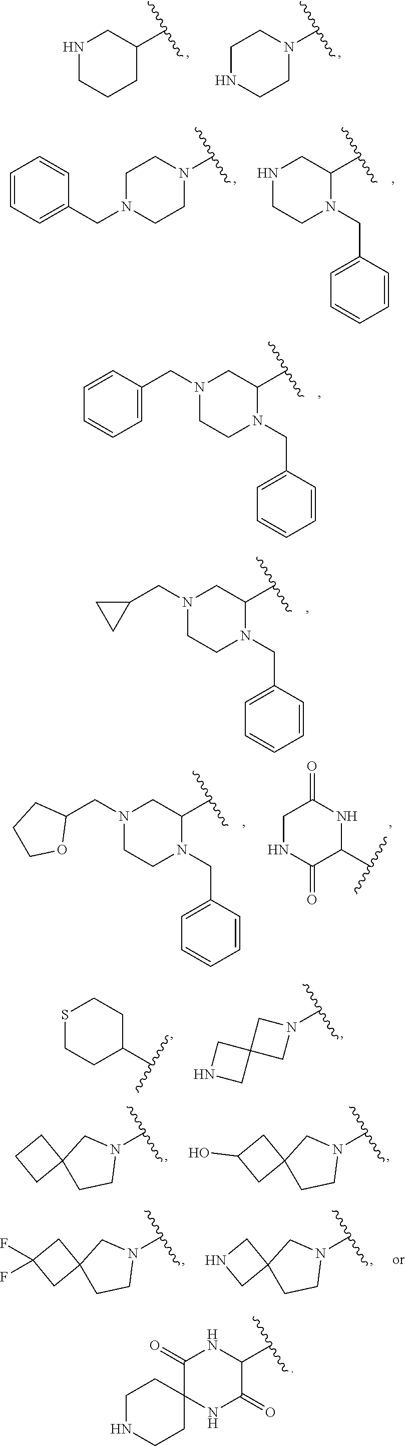

In some embodiments R¹ is

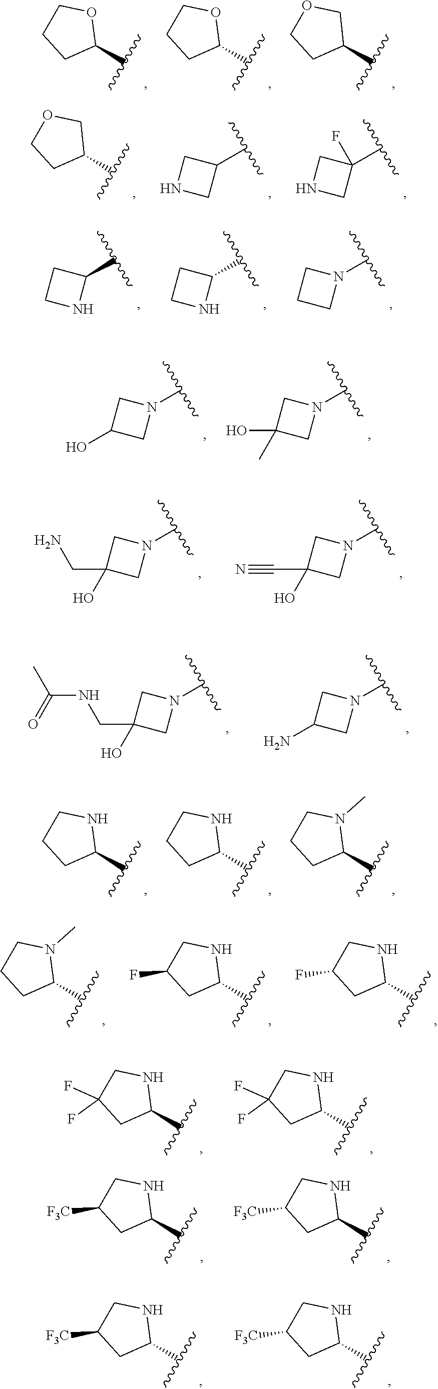

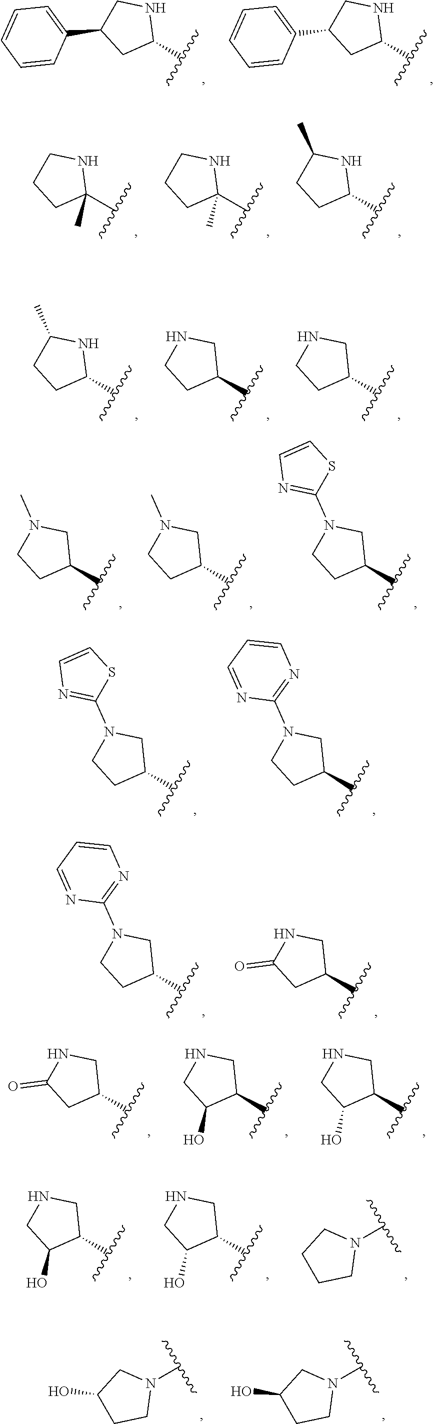

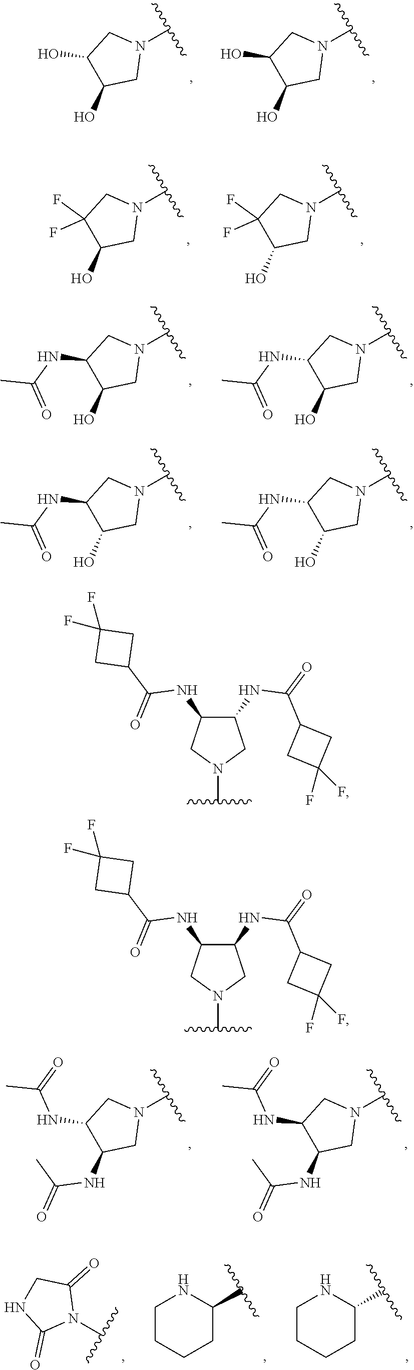

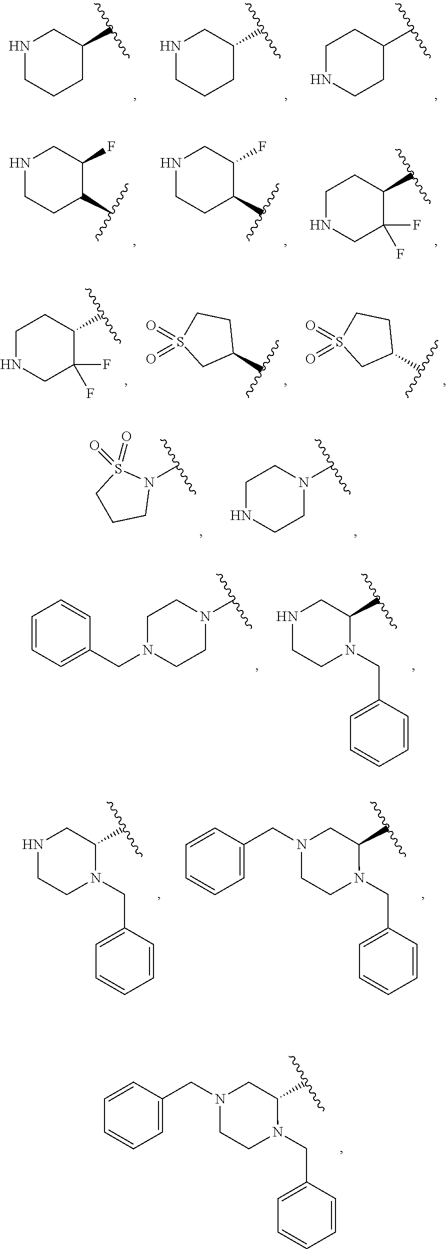

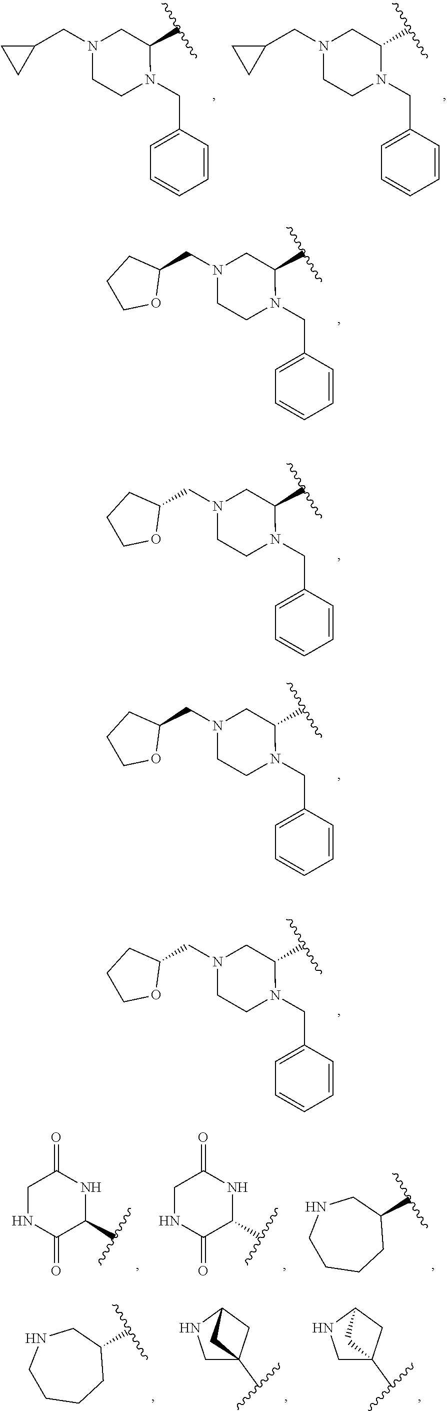

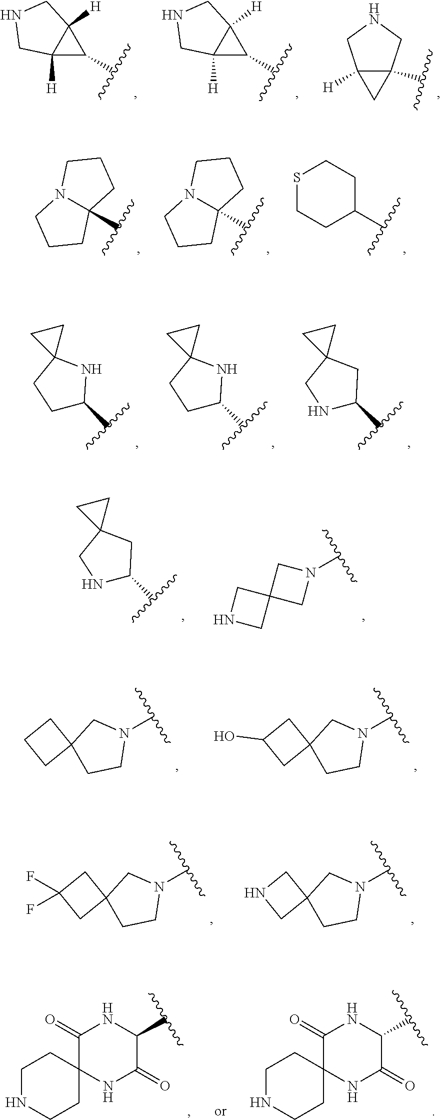

In some embodiments, R¹ is

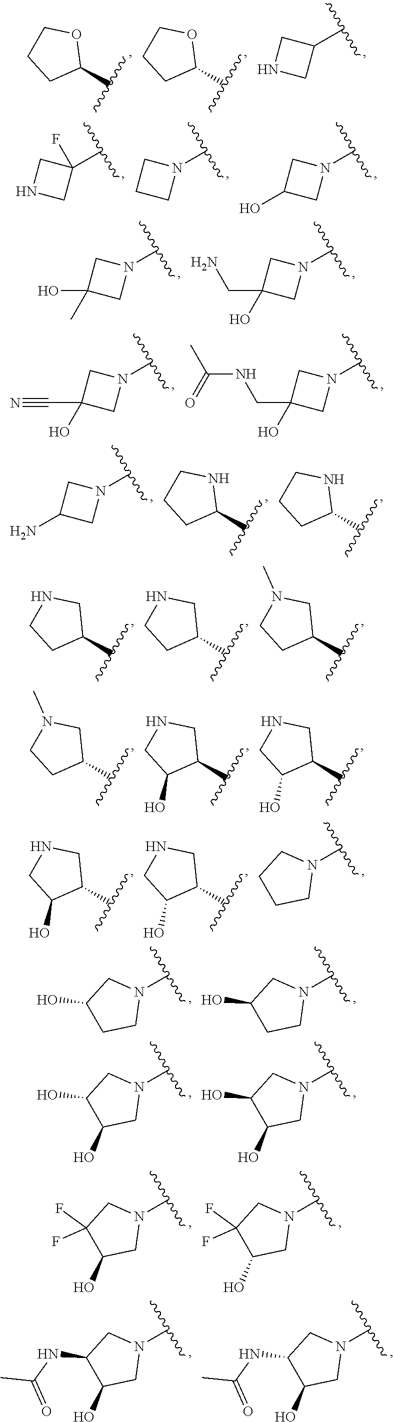

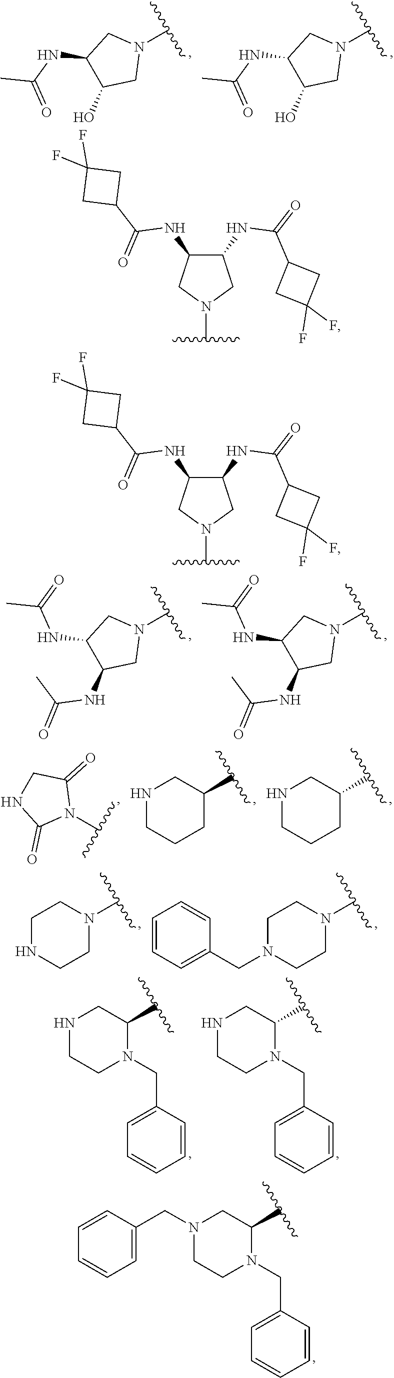

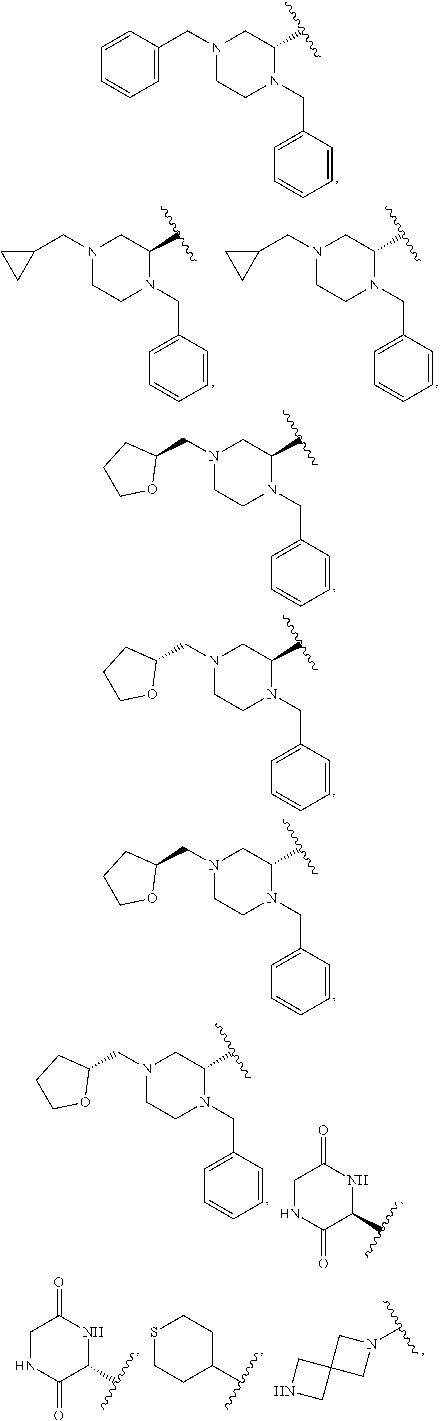

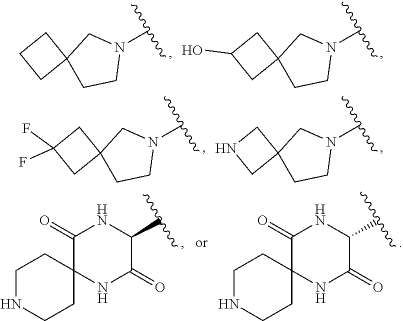

In some embodiments R¹ is

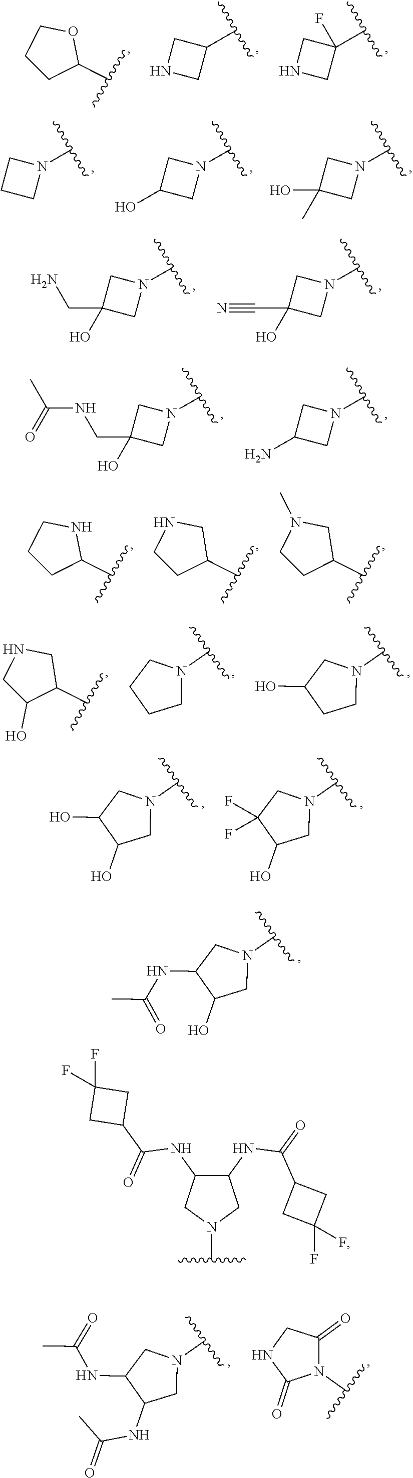

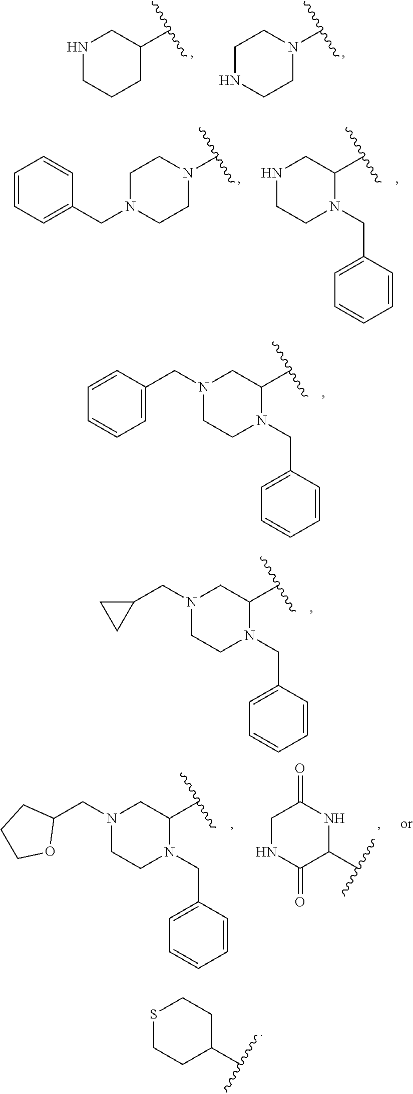

In some embodiments R¹ is

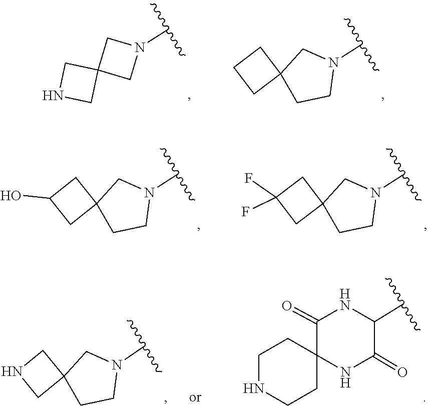 [SMILES: *N1CC2(CNC2)C1 ; *N1CCC2(CCC2)C1 ; *N1CCC2(CC(O)C2)C1 ; *N1CCC2(C1)CC(F)(F)C2 ; *N1CCC2(CNC2)C1 ; *C1NC(=O)C2(CCNCC2)NC1=O]

In some embodiments, R¹ is

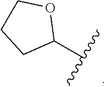 [SMILES: *C1CCCO1]

In some embodiments, R¹ is

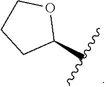 [SMILES: *[C@H]1CCCO1]

In some embodiments, R¹ is

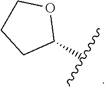 [SMILES: *[C@@H]1CCCO1]

In some embodiments, R¹ is

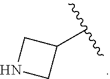 [SMILES: *C1CNC1]

In some embodiments, R¹ is

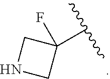 [SMILES: *C1(F)CNC1]

In some embodiments, R¹ is

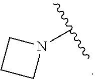 [SMILES: *N1CCC1]

In some embodiments, R¹ is

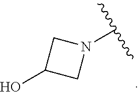 [SMILES: *N1CC(O)C1]

In some embodiments, R¹ is

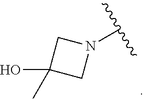 [SMILES: *N1CC(C)(O)C1]

In some embodiments, R¹ is

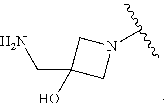 [SMILES: *N1CC(O)(CN)C1]

In some embodiments, R¹ is

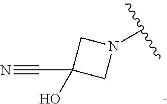 [SMILES: *N1CC(O)(C#N)C1]

In some embodiments, R¹ is

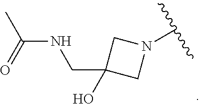 [SMILES: *N1CC(O)(CNC(C)=O)C1]

In some embodiments, R¹ is

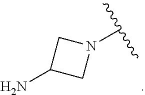 [SMILES: *N1CC(N)C1]

In some embodiments, R¹ is

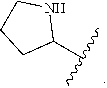 [SMILES: *C1CCCN1]

In some embodiments, R¹ is

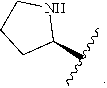 [SMILES: *[C@H]1CCCN1]

In some embodiments, R¹ is

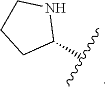 [SMILES: *[C@@H]1CCCN1]

In some embodiments, R¹ is

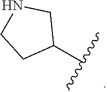 [SMILES: *C1CCNC1]

In some embodiments, R¹ is

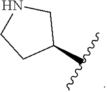 [SMILES: *[C@H]1CCNC1]

In some embodiments, R¹ is

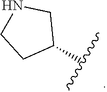 [SMILES: *[C@@H]1CCNC1]

In some embodiments, R¹ is

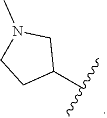 [SMILES: *C1CCN(C)C1]

In some embodiments, R¹ is

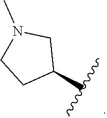 [SMILES: *[C@H]1CCN(C)C1]

In some embodiments, R¹ is

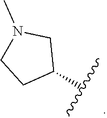 [SMILES: *[C@@H]1CCN(C)C1]

In some embodiments, R¹ is

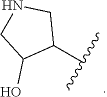 [SMILES: *C1CNCC1O]

In some embodiments, R¹ is

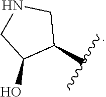 [SMILES: *[C@@H]1CNC[C@@H]1O]

In some embodiments, R¹ is

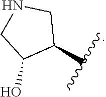 [SMILES: *[C@@H]1CNC[C@H]1O]

In some embodiments, R¹ is

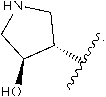 [SMILES: *[C@H]1CNC[C@@H]1O]

In some embodiments, R¹ is

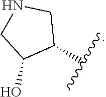 [SMILES: *[C@H]1CNC[C@H]1O]

In some embodiments, R¹ is

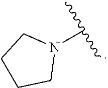 [SMILES: *N1CCCC1]

In some embodiments, R¹ is

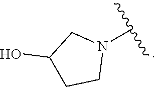 [SMILES: *N1CCC(O)C1]

In some embodiments, R¹ is

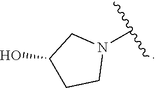 [SMILES: *N1CC[C@H](O)C1]

In some embodiments, R¹ is

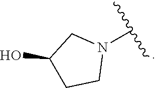 [SMILES: *N1CC[C@@H](O)C1]

In some embodiments, R¹ is

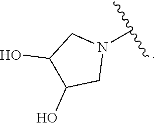 [SMILES: *N1CC(O)C(O)C1]

In some embodiments, R¹ is

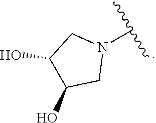 [SMILES: *N1C[C@@H](O)[C@H](O)C1]

In some embodiments, R¹ is

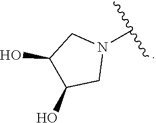 [SMILES: *N1C[C@@H](O)[C@@H](O)C1]

In some embodiments, R¹ is

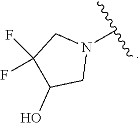 [SMILES: *N1CC(O)C(F)(F)C1]

In some embodiments, R¹ is

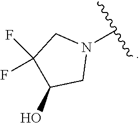 [SMILES: *N1C[C@@H](O)C(F)(F)C1]

In some embodiments, R¹ is

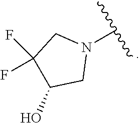 [SMILES: *N1C[C@H](O)C(F)(F)C1]

In some embodiments, R¹ is

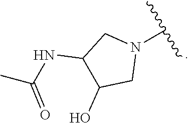 [SMILES: *N1CC(O)C(NC(C)=O)C1]

In some embodiments, R¹ is

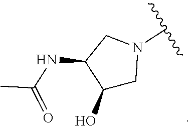 [SMILES: *N1C[C@@H](O)[C@@H](NC(C)=O)C1]

In some embodiments, R¹ is

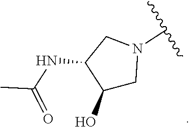 [SMILES: *N1C[C@@H](O)[C@H](NC(C)=O)C1]

In some embodiments, R¹ is

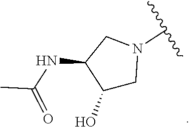 [SMILES: *N1C[C@H](NC(C)=O)[C@@H](O)C1]

In some embodiments, R¹ is

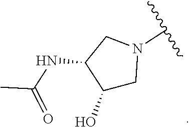 [SMILES: *N1C[C@H](O)[C@H](NC(C)=O)C1]

In some embodiments, R¹ is

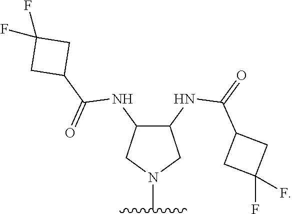 [SMILES: *N1CC(NC(=O)C2CC(F)(F)C2)C(NC(=O)C2CC(F)(F)C2)C1]

In some embodiments, R¹ is

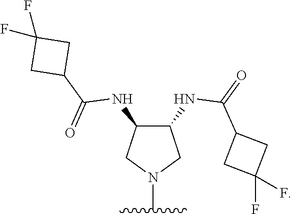 [SMILES: *N1C[C@@H](NC(=O)C2CC(F)(F)C2)[C@H](NC(=O)C2CC(F)(F)C2)C1]

In some embodiments, R¹ is

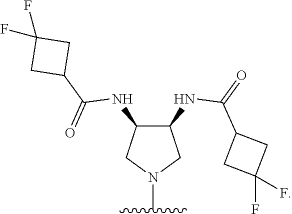 [SMILES: *N1C[C@H](NC(=O)C2CC(F)(F)C2)[C@H](NC(=O)C2CC(F)(F)C2)C1]

In some embodiments, R¹ is

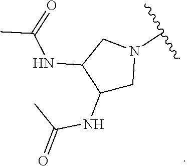 [SMILES: *N1CC(NC(C)=O)C(NC(C)=O)C1]

In some embodiments, R¹ is

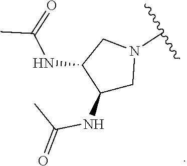 [SMILES: *N1C[C@@H](NC(C)=O)[C@H](NC(C)=O)C1]

In some embodiments, R¹ is

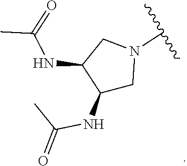 [SMILES: *N1C[C@H](NC(C)=O)[C@H](NC(C)=O)C1]

In some embodiments, R¹ is

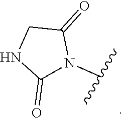 [SMILES: *N1C(=O)CNC1=O]

In some embodiments, R¹ is

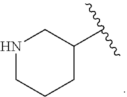 [SMILES: *C1CCCNC1]

In some embodiments, R¹ is

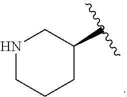 [SMILES: *[C@H]1CCCNC1]

In some embodiments, R¹ is

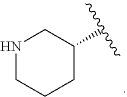 [SMILES: *[C@@H]1CCCNC1]

In some embodiments, R¹ is

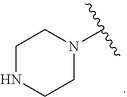 [SMILES: *N1CCNCC1]

In some embodiments, R¹ is

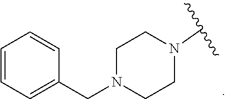 [SMILES: *N1CCN(Cc2ccccc2)CC1]

In some embodiments, R¹ is

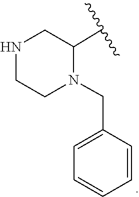 [SMILES: *C1CNCCN1Cc1ccccc1]

In some embodiments, R¹ is

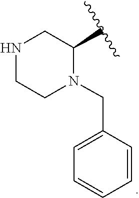 [SMILES: *[C@@H]1CNCCN1Cc1ccccc1]

In some embodiments, R¹ is

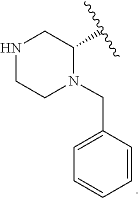 [SMILES: *[C@H]1CNCCN1Cc1ccccc1]

In some embodiments, R¹ is

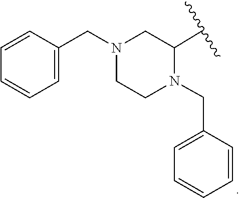 [SMILES: *C1CN(Cc2ccccc2)CCN1Cc1ccccc1]

In some embodiments, R¹ is

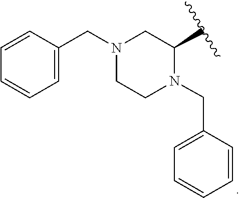 [SMILES: *[C@@H]1CN(Cc2ccccc2)CCN1Cc1ccccc1]

In some embodiments, R¹ is

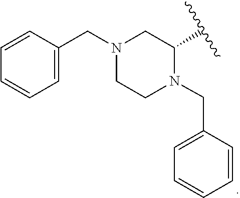 [SMILES: *[C@H]1CN(Cc2ccccc2)CCN1Cc1ccccc1]

In some embodiments, R¹ is

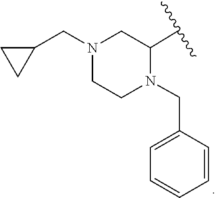 [SMILES: *C1CN(CC2CC2)CCN1Cc1ccccc1]

In some embodiments, R¹ is

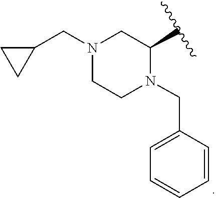 [SMILES: *[C@@H]1CN(CC2CC2)CCN1Cc1ccccc1]

In some embodiments, R¹ is

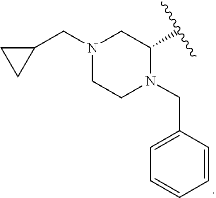 [SMILES: *[C@H]1CN(CC2CC2)CCN1Cc1ccccc1]

In some embodiments, R¹ is

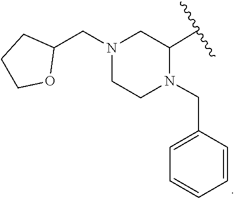 [SMILES: *C1CN(CC2CCCO2)CCN1Cc1ccccc1]

In some embodiments, R¹ is

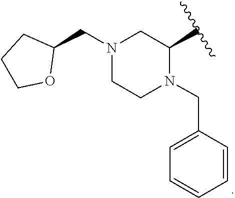 [SMILES: *[C@@H]1CN(C[C@@H]2CCCO2)CCN1Cc1ccccc1]

In some embodiments, R¹ is

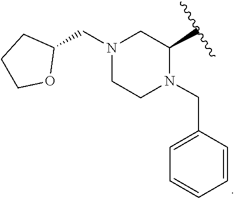 [SMILES: *[C@@H]1CN(C[C@H]2CCCO2)CCN1Cc1ccccc1]

In some embodiments, R¹ is

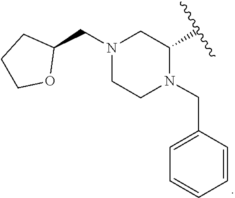 [SMILES: *[C@H]1CN(C[C@@H]2CCCO2)CCN1Cc1ccccc1]

In some embodiments, R¹ is

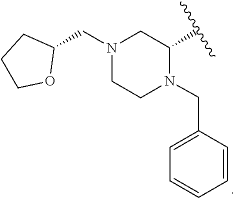 [SMILES: *[C@H]1CN(C[C@H]2CCCO2)CCN1Cc1ccccc1]

In some embodiments, R¹ is

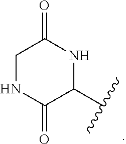 [SMILES: *C1NC(=O)CNC1=O]

In some embodiments, R¹ is

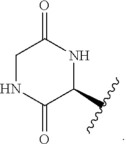 [SMILES: *[C@@H]1NC(=O)CNC1=O]

In some embodiments, R¹ is

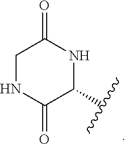 [SMILES: *[C@H]1NC(=O)CNC1=O]

In some embodiments, R¹ is

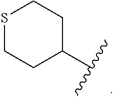 [SMILES: *C1CCSCC1]

In some embodiments, R¹ is

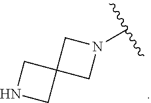 [SMILES: *N1CC2(CNC2)C1]

In some embodiments, R¹ is

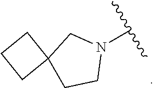 [SMILES: *N1CCC2(CCC2)C1]

In some embodiments, R¹ is

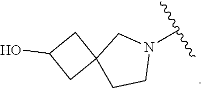 [SMILES: *N1CCC2(CC(O)C2)C1]

In some embodiments, R¹ is

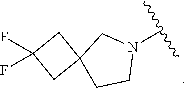 [SMILES: *N1CCC2(C1)CC(F)(F)C2]

In some embodiments, R¹ is

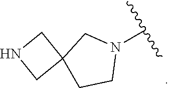 [SMILES: *N1CCC2(CNC2)C1]

In some embodiments, R¹ is or

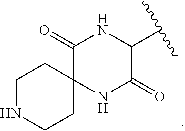 [SMILES: *C1NC(=O)C2(CCNCC2)NC1=O]

In some embodiments, R¹ is

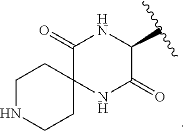 [SMILES: *[C@@H]1NC(=O)C2(CCNCC2)NC1=O]

In some embodiments, R¹ is

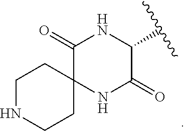 [SMILES: *[C@H]1NC(=O)C2(CCNCC2)NC1=O]

In some embodiments, R¹ is optionally substituted aryl or optionally substituted heteroaryl. In some embodiments, R¹ is optionally substituted aryl. In some embodiments, R¹ is optionally substituted C₆₋₁₄ aryl. In some embodiments, R¹ is optionally substituted phenyl. In some embodiments, R¹ is unsubstituted phenyl. In some embodiments, R¹ is substituted phenyl. In some embodiments, R¹ is phenyl substituted with halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂. In some embodiments, R¹ is phenyl substituted with halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —OR^{1a}, and/or —N(R^{1a})₂. In some embodiments, R¹ is phenyl optionally substituted with halogen, optionally substituted alkyl, optionally substituted aryl, —OR^{1a}, —N(R^{1a})₂, —C(═NR^{A})N(R^{A})₂, and/or —B(OR^{A})₂. In some embodiments, R¹ is phenyl substituted with halogen. In some embodiments, R¹ is phenyl substituted with optionally substituted alkyl. In some embodiments, R¹ is phenyl substituted with optionally substituted C₁₋₆ alkyl. In some embodiments, R¹ is phenyl substituted with optionally substituted phenyl. In some embodiments, R¹ is phenyl substituted with —OR^{1a}. In some embodiments, R¹ is phenyl substituted with —N(R^{1a})₂. In some embodiments, R¹ is phenyl substituted with —C(═NR^{A})N(R^{A})₂. In some embodiments, R¹ is phenyl substituted with —B(OR^{A})₂.

In some embodiments, R¹ is of formula:

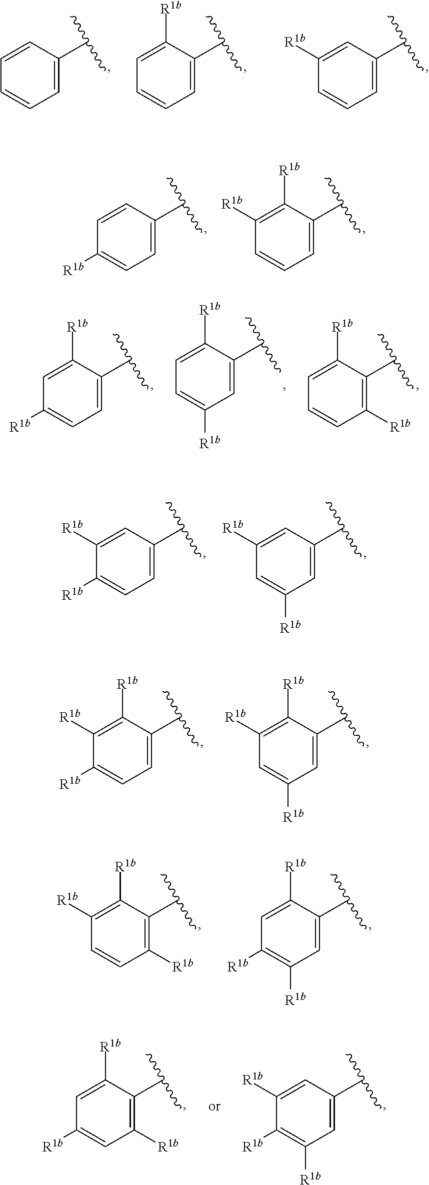

wherein each instance of R^{1b} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In some embodiments, R¹ is

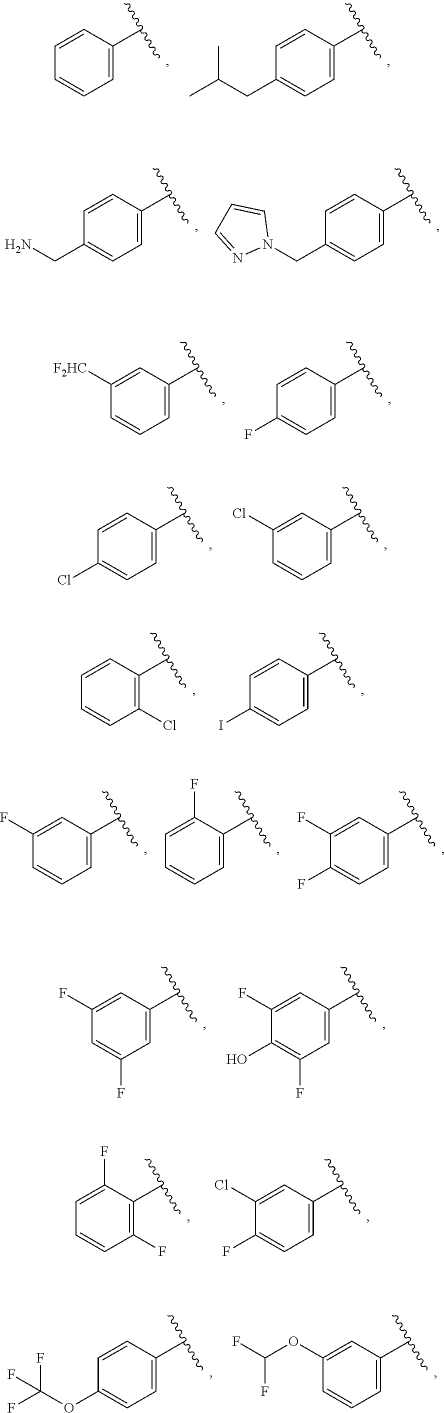

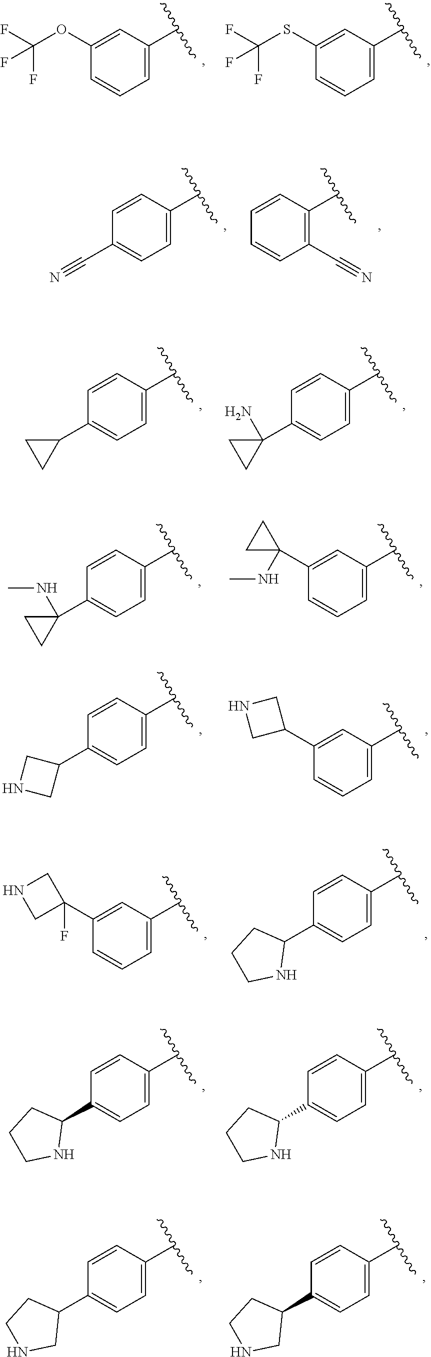

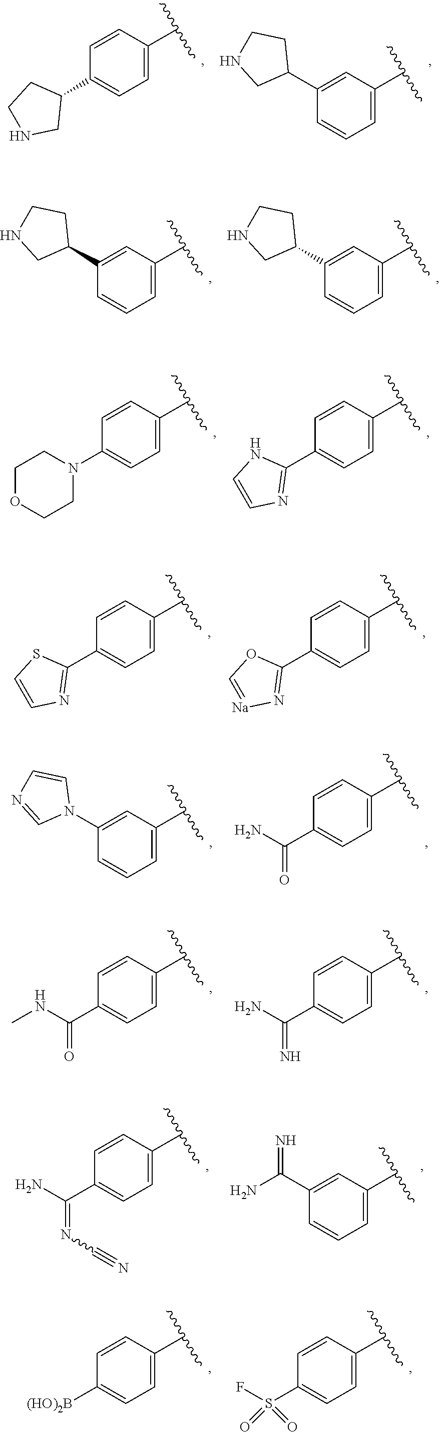

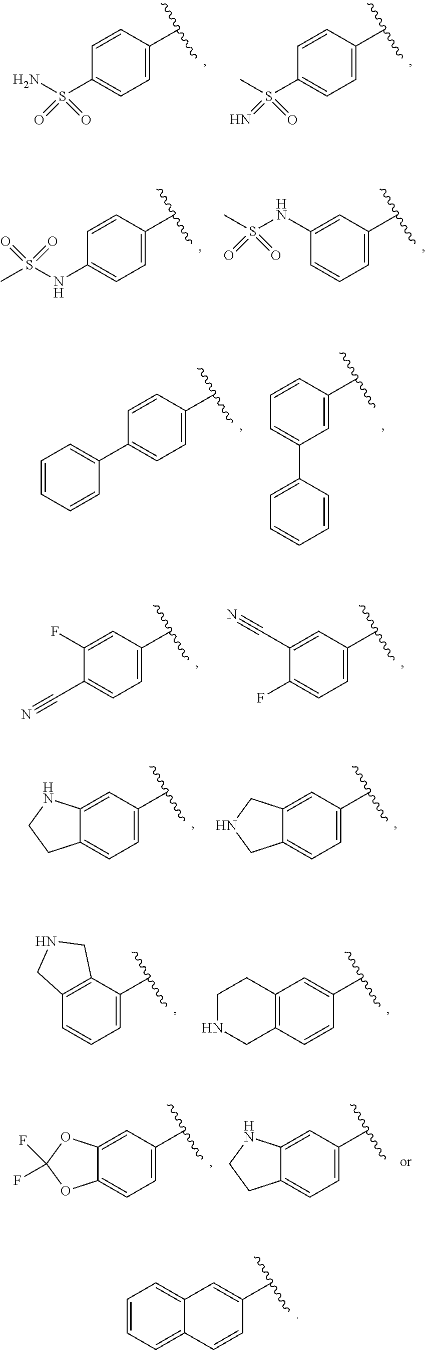

In some embodiments, R¹ is

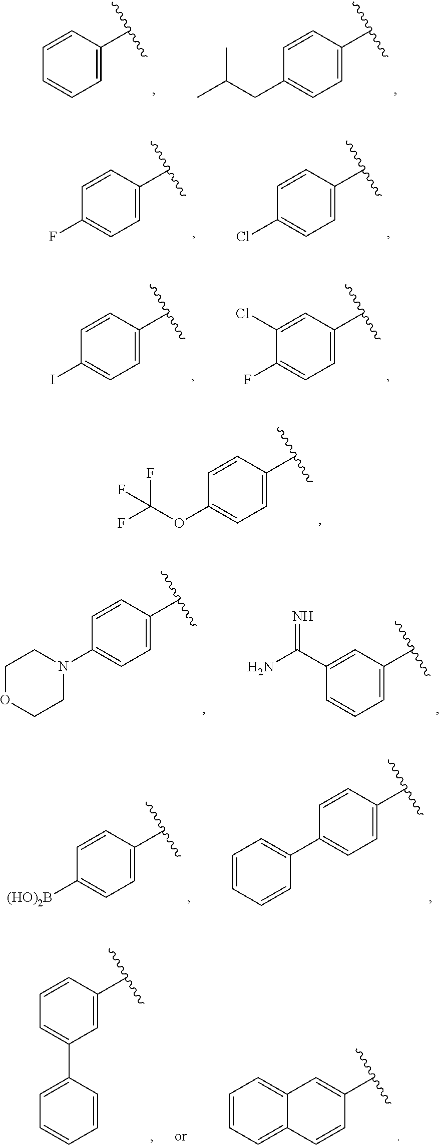

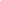

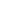

In some embodiments, R¹ is

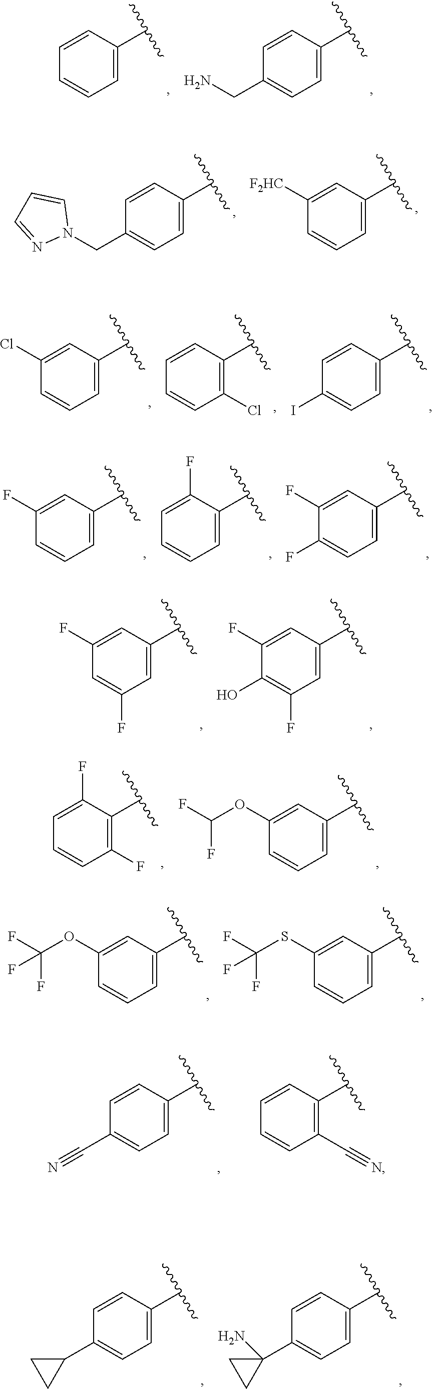

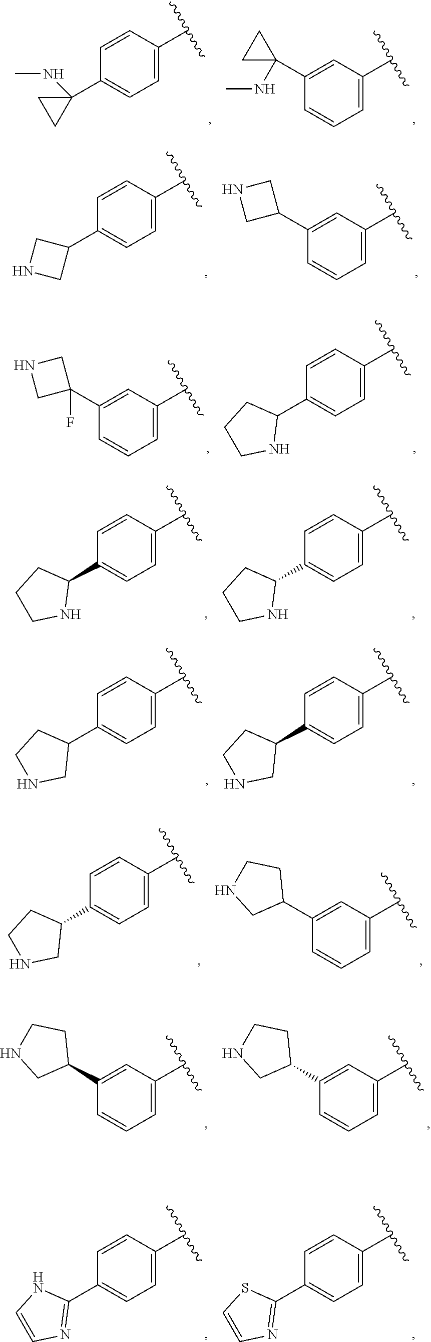

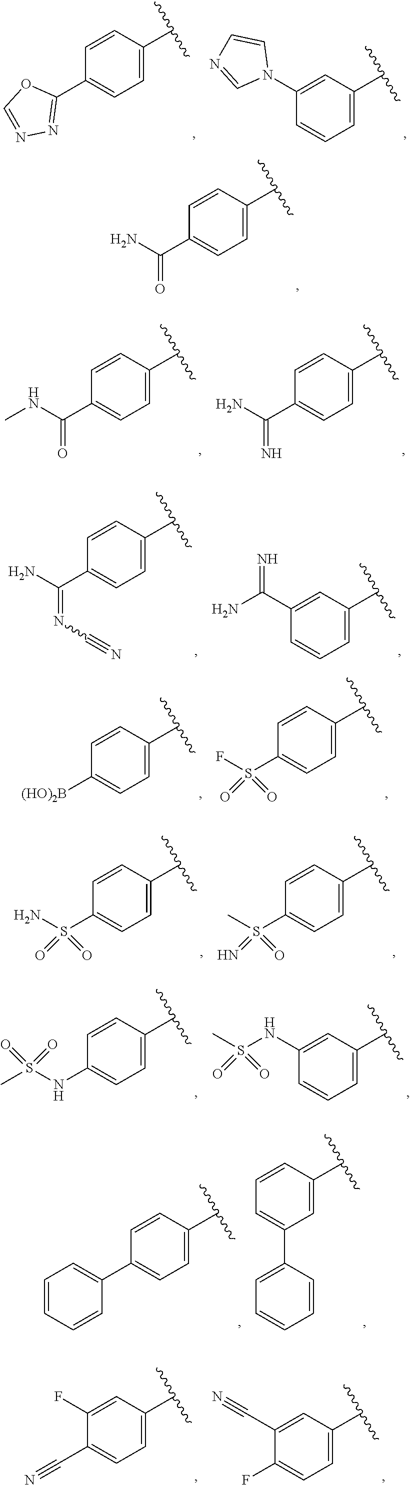

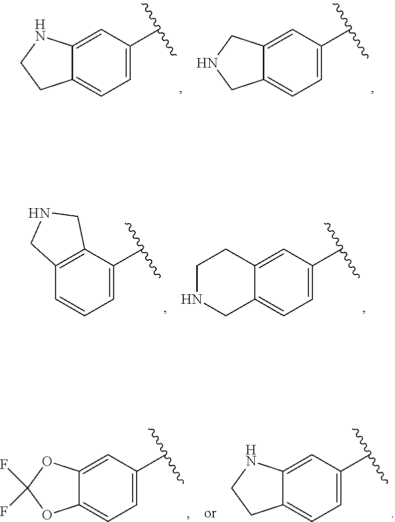 [SMILES: *c1ccc2c(c1)NCC2 ; *c1ccc2c(c1)CNC2 ; *c1cccc2c1CNC2 ; *c1ccc2c(c1)CCNC2 ; *c1ccc2c(c1)OC(F)(F)O2 ; *c1ccc2c(c1)NCC2]

In some embodiments, R¹ is

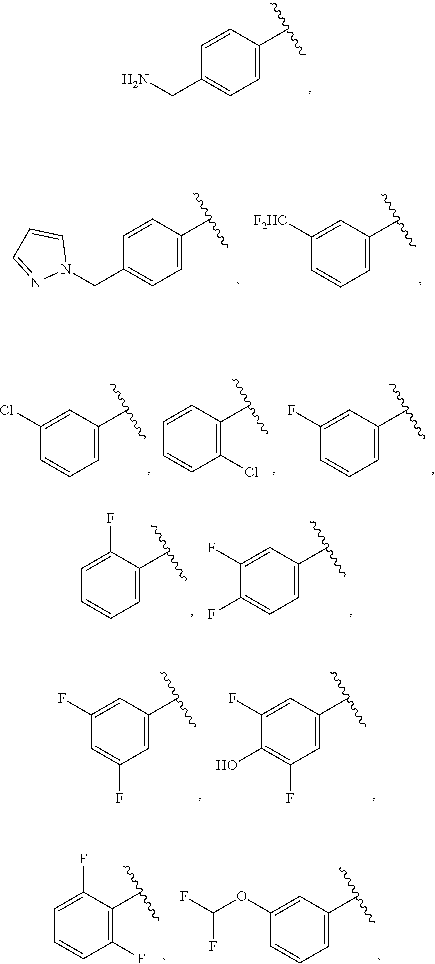

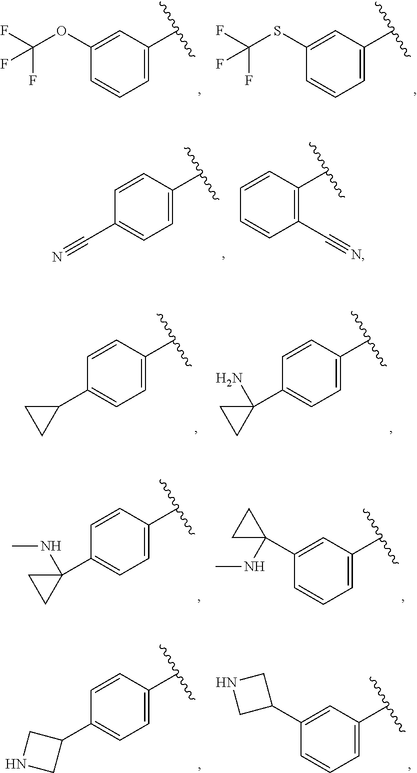

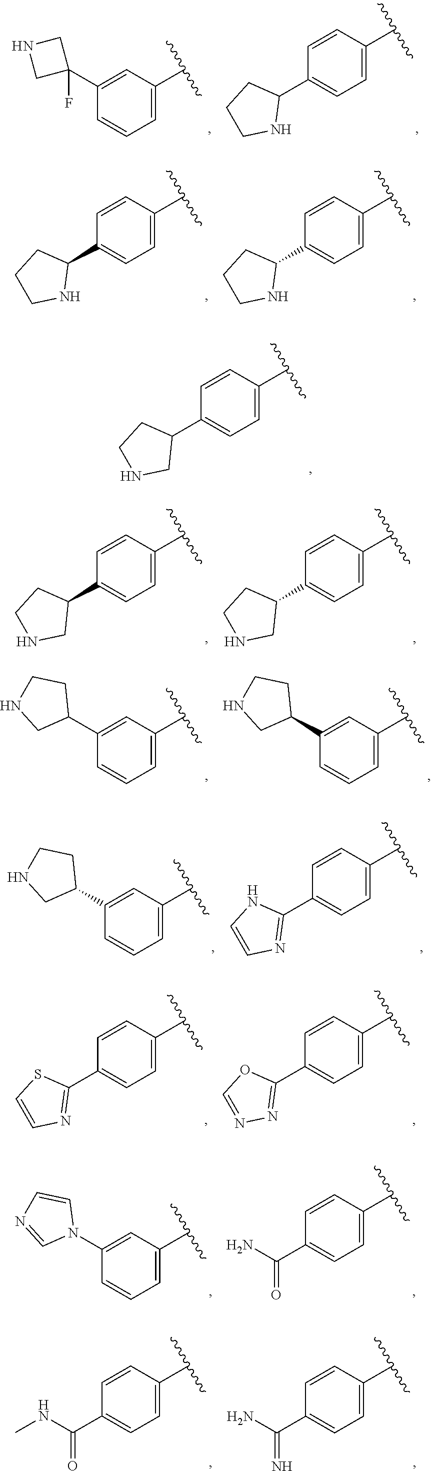

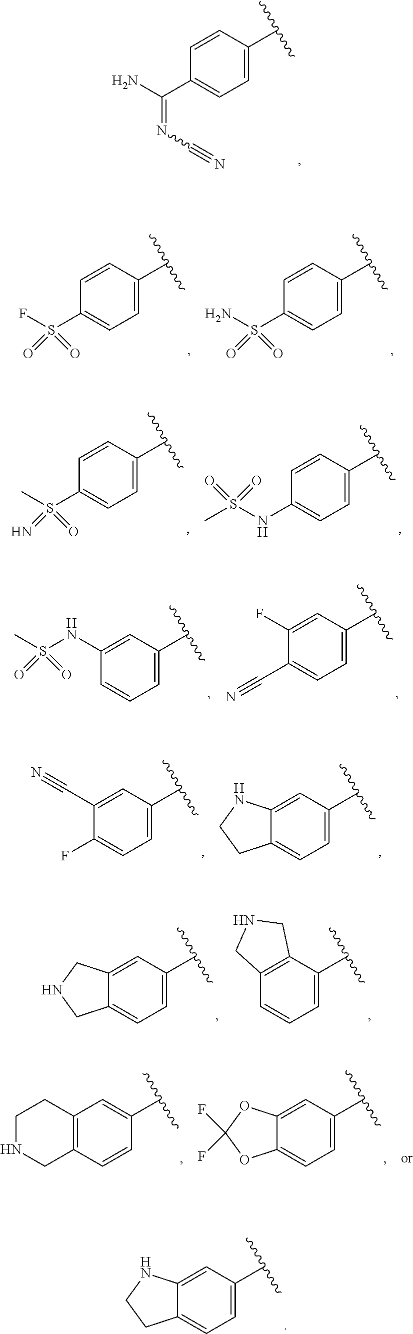

or In some embodiments, R¹ is

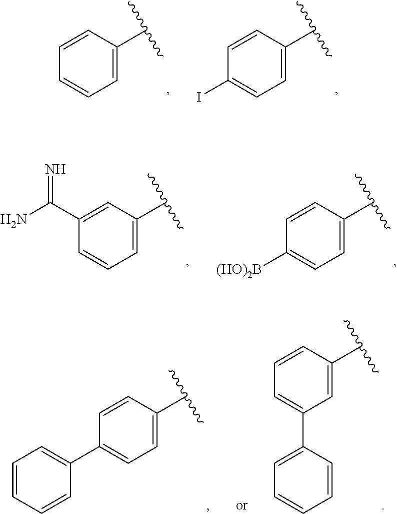 [SMILES: *c1ccccc1 ; *c1ccc(I)cc1 ; *c1cccc(C(=N)N)c1 ; *c1ccc(B(O)O)cc1 ; *c1ccc(-c2ccccc2)cc1 ; *c1cccc(-c2ccccc2)c1]

In some embodiments, R¹ is

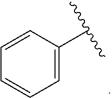 [SMILES: *c1ccccc1]

In some embodiments, R¹ is

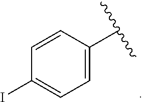 [SMILES: *c1ccc(I)cc1]

In some embodiments, R¹ is

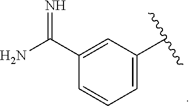 [SMILES: *c1cccc(C(=N)N)c1]

In some embodiments, R¹ is

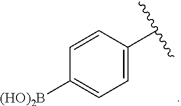 [SMILES: *c1ccc(B(O)O)cc1]

In some embodiments, R¹ is

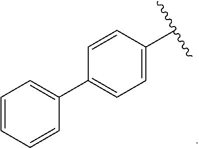 [SMILES: *c1ccc(-c2ccccc2)cc1]

In some embodiments, R¹ is

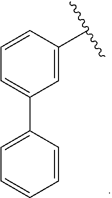 [SMILES: *c1cccc(-c2ccccc2)c1]

In some embodiments, R¹ is optionally substituted heteroaryl. In some embodiments, R¹ is optionally substituted 4-10 membered heteroaryl. In some embodiments, R¹ is optionally substituted 5-6 membered heteroaryl. In some embodiments, R¹ is optionally substituted monocyclic 5-6 membered heteroaryl. In some embodiments, R¹ is optionally substituted pyridyl, optionally substituted pyrazinyl, optionally substituted pyrimidinyl, optionally substituted pyridazinyl, optionally substituted pyrazolyl, optionally substituted imidazolyl, optionally substituted triazolyl, optionally substituted tetrazolyl, optionally substituted benzimidazolyl, optionally substituted benzotriazolyl, optionally substituted purinyl, optionally substituted thiophenyl, optionally substituted benzothiophenyl, optionally substituted oxazolyl, optionally substituted isoxazolyl, optionally substituted oxadiazolyl, optionally substituted benzooxadiazolyl, optionally substituted thiazolyl, optionally substituted thiadiazolyl, or optionally substituted benzothiadiazolyl. In some embodiments, R¹ is substituted pyridyl, substituted pyrazinyl, substituted pyrimidinyl, substituted pyridazinyl, substituted pyrazolyl, substituted imidazolyl, substituted triazolyl, substituted tetrazolyl, substituted benzimidazolyl, substituted benzotriazolyl, substituted purinyl, substituted thiophenyl, substituted benzothiophenyl, substituted oxazolyl, substituted isoxazolyl, substituted oxadiazolyl, substituted benzooxadiazolyl, substituted thiazolyl, substituted thiadiazolyl, or substituted benzothiadiazolyl. In some embodiments, R¹ is unsubstituted pyridyl, unsubstituted pyrazinyl, unsubstituted pyrimidinyl, unsubstituted pyridazinyl, unsubstituted pyrazolyl, unsubstituted imidazolyl, unsubstituted triazolyl, unsubstituted tetrazolyl, unsubstituted benzimidazolyl, unsubstituted benzotriazolyl, unsubstituted purinyl, unsubstituted thiophenyl, unsubstituted benzothiophenyl, unsubstituted oxazolyl, unsubstituted isoxazolyl, unsubstituted oxadiazolyl, unsubstituted benzooxadiazolyl, unsubstituted thiazolyl, unsubstituted thiadiazolyl, or unsubstituted benzothiadiazolyl. In some embodiments, R¹ is optionally substituted pyridyl. In some embodiments, R¹ is optionally substituted pyrazinyl. In some embodiments, R¹ is optionally substituted pyrimidinyl. In some embodiments, R¹ is optionally substituted pyridazinyl. In some embodiments, R¹ is optionally substituted pyrazolyl. In some embodiments, R¹ is optionally substituted imidazolyl. In some embodiments, R¹ is optionally substituted triazolyl. In some embodiments, R¹ is optionally substituted tetrazolyl. In some embodiments, R¹ is optionally substituted benzimidazolyl. In some embodiments, R¹ is optionally substituted benzotriazolyl. In some embodiments, R¹ is optionally substituted purinyl. In some embodiments, R¹ is optionally substituted thiophenyl. In some embodiments, R¹ is optionally substituted benzothiophenyl. In some embodiments, R¹ is optionally substituted oxazolyl. In some embodiments, R¹ is optionally substituted isoxazolyl. In some embodiments, R¹ is optionally substituted oxadiazolyl. In some embodiments, R¹ is optionally substituted benzooxadiazolyl. In some embodiments, R¹ is optionally substituted thiazolyl. In some embodiments, R¹ is optionally substituted thiadiazolyl. In some embodiments, R¹ is optionally substituted benzothiadiazolyl.

In some embodiments, R¹ is optionally substituted pyridyl, optionally substituted pyrimidinyl, optionally substituted pyridazinyl, optionally substituted pyrazolyl, optionally substituted imidazolyl, optionally substituted triazolyl, optionally substituted tetrazolyl, optionally substituted benzimidazolyl, optionally substituted purinyl, optionally substituted oxazolyl, optionally substituted isoxazolyl, or optionally substituted thiazolyl. In some embodiments, R¹ is substituted pyridyl, substituted pyrimidinyl, substituted pyridazinyl, substituted pyrazolyl, substituted imidazolyl, substituted triazolyl, substituted tetrazolyl, substituted benzimidazolyl, substituted purinyl, substituted oxazolyl, substituted isoxazolyl, or substituted thiazolyl. In some embodiments, R¹ is unsubstituted pyridyl, unsubstituted pyrimidinyl, unsubstituted pyridazinyl, unsubstituted pyrazolyl, unsubstituted imidazolyl, unsubstituted triazolyl, unsubstituted tetrazolyl, unsubstituted benzimidazolyl, unsubstituted purinyl, unsubstituted oxazolyl, unsubstituted isoxazolyl, or unsubstituted thiazolyl.

In some embodiments, R¹ is of formula:

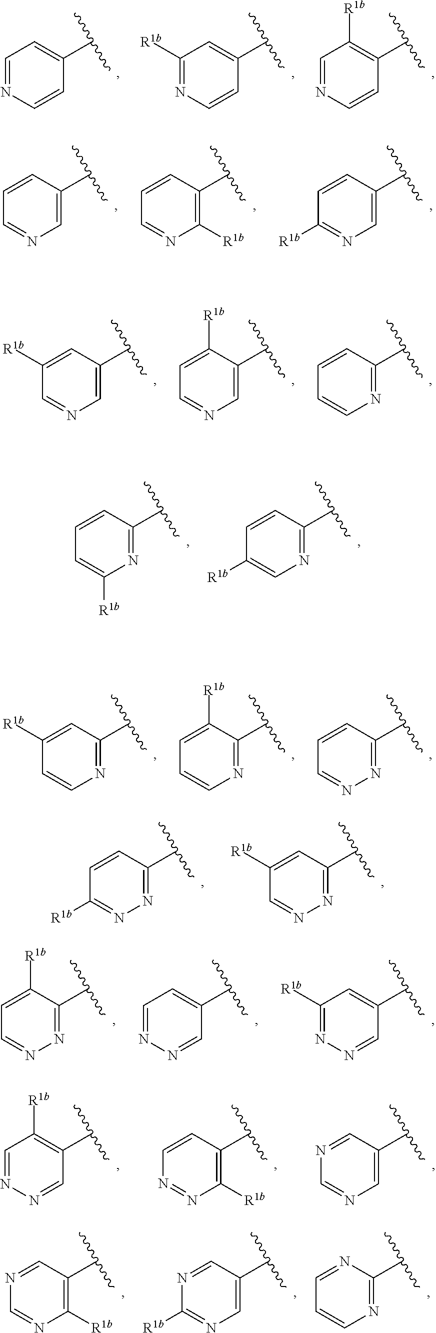

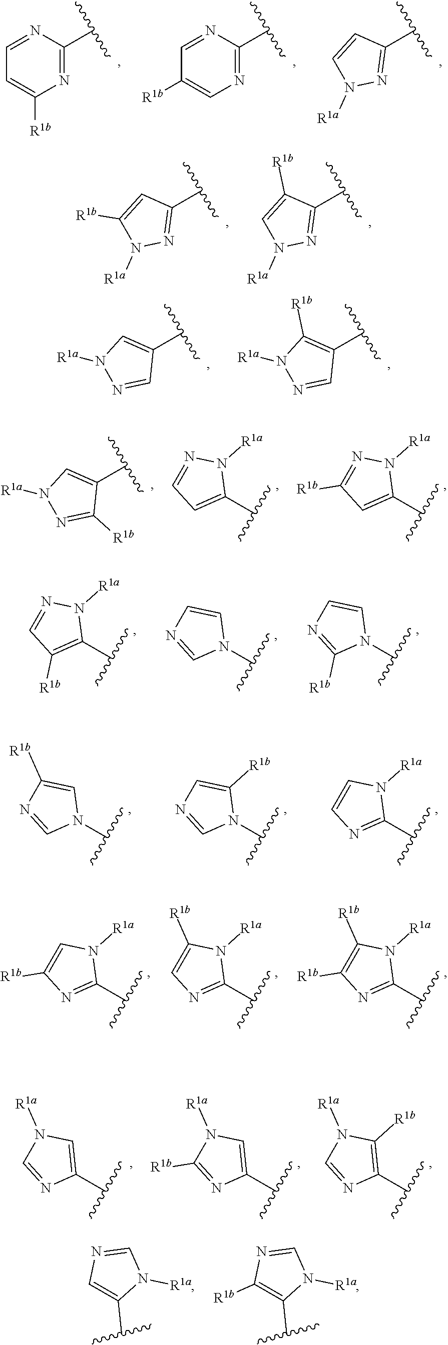

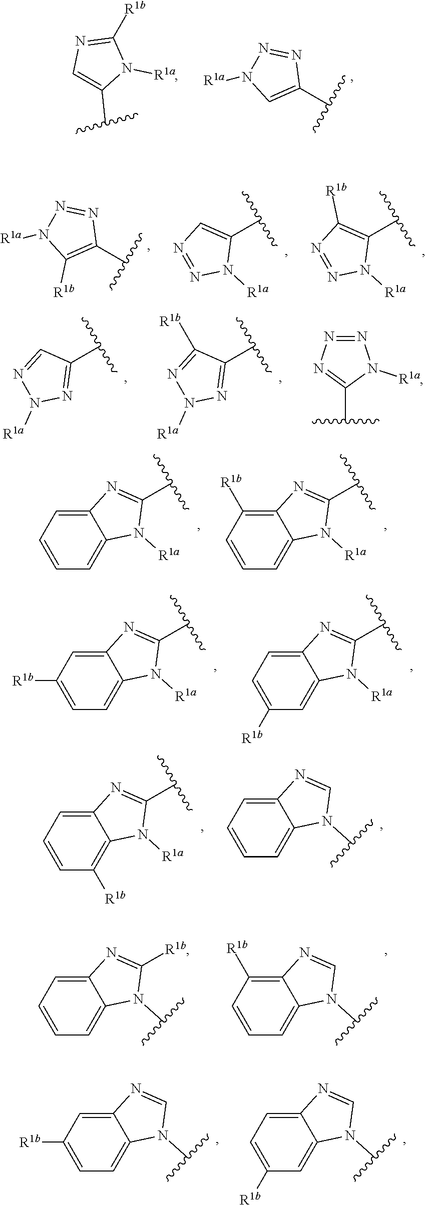

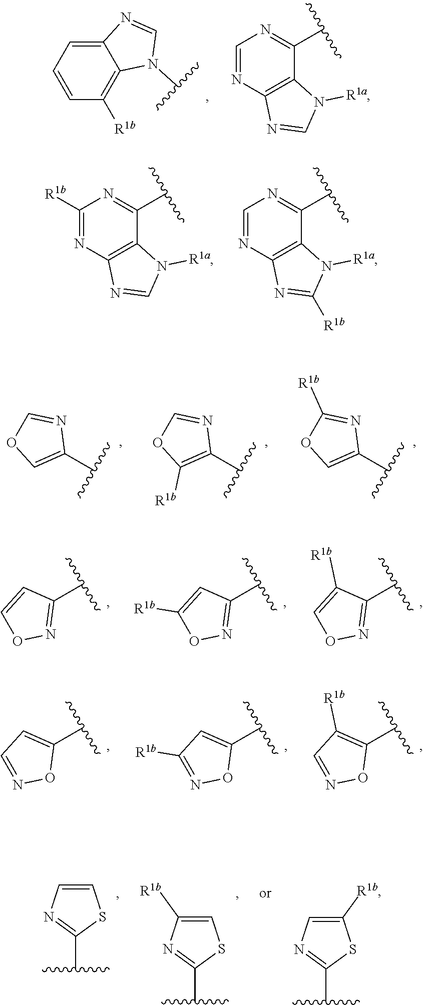

wherein:
each instance of R^{1a} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group; and
each instance of R^{1b} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In some embodiments, R¹ is

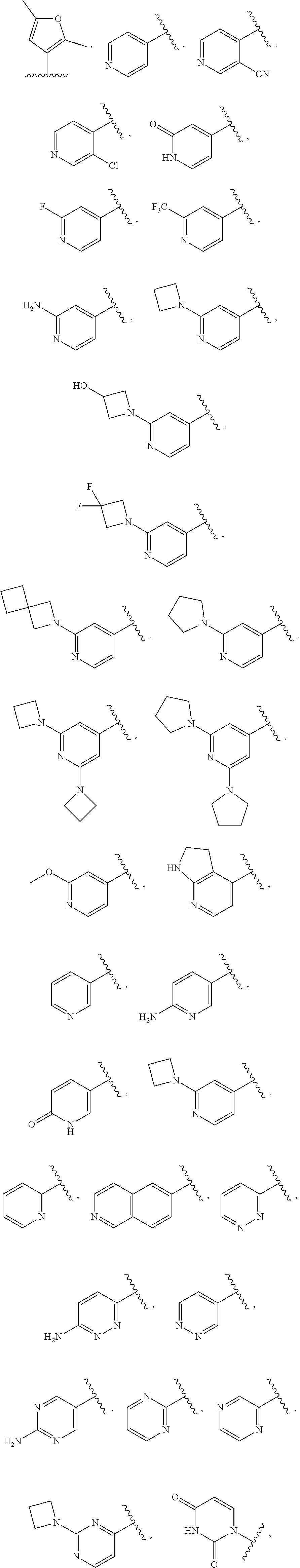

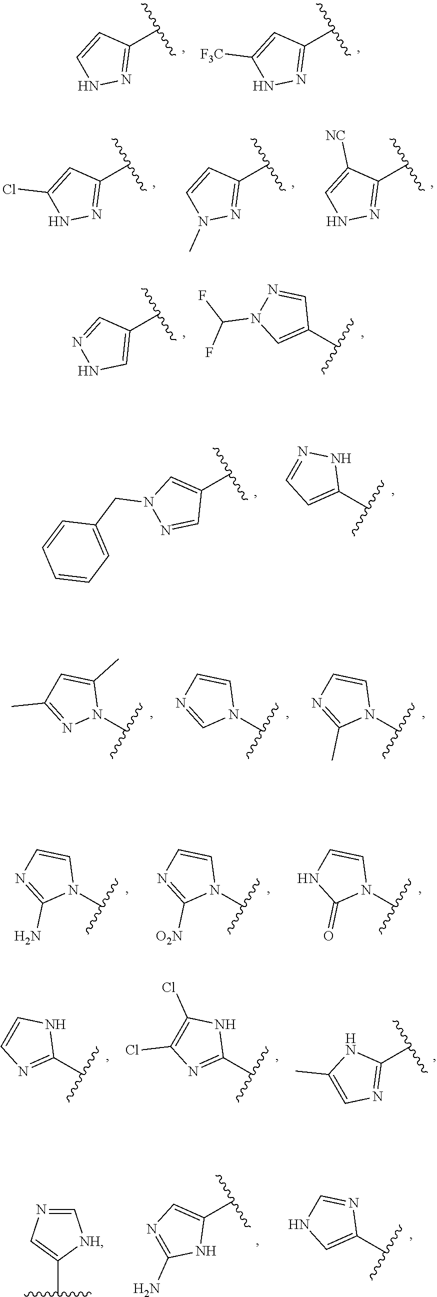

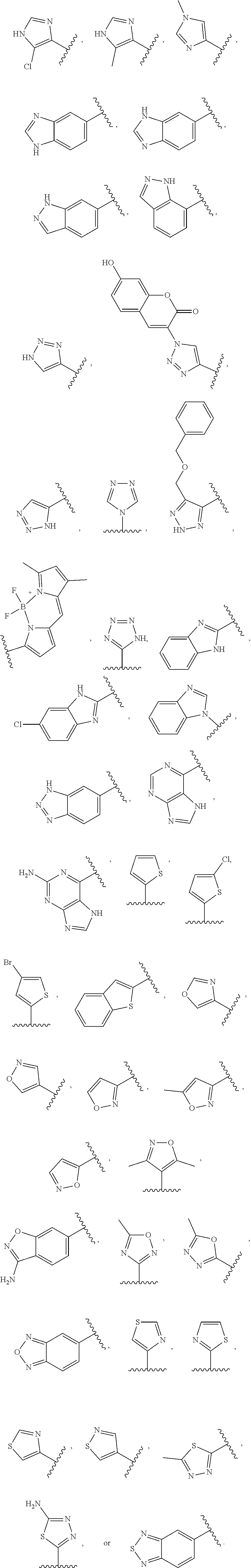

In some embodiments, R¹ is

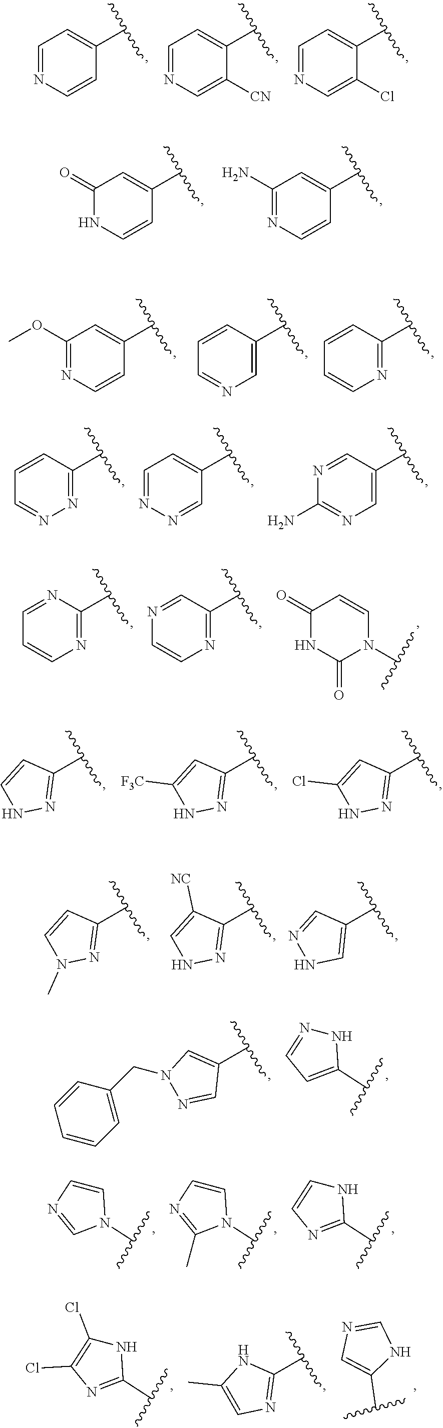

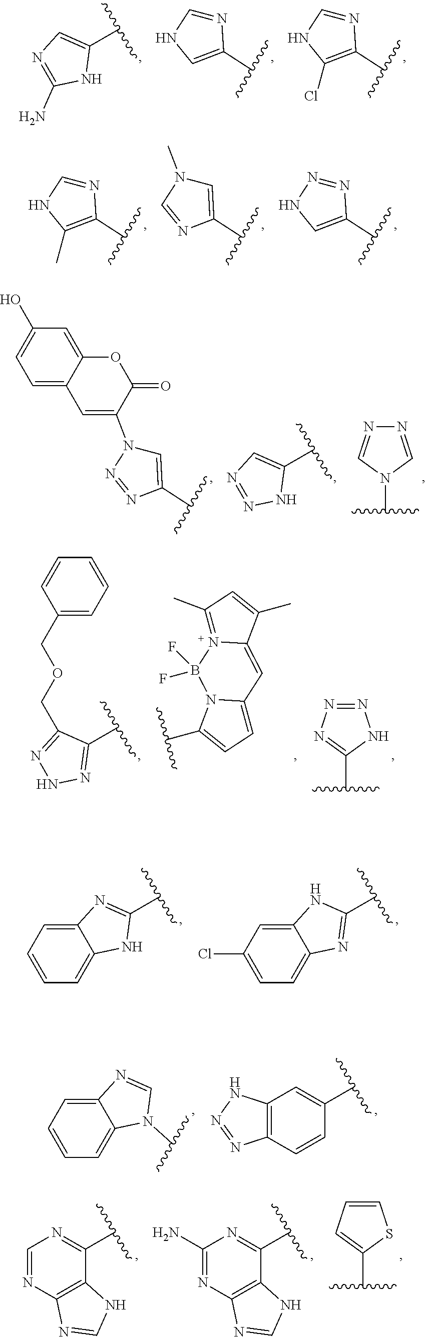

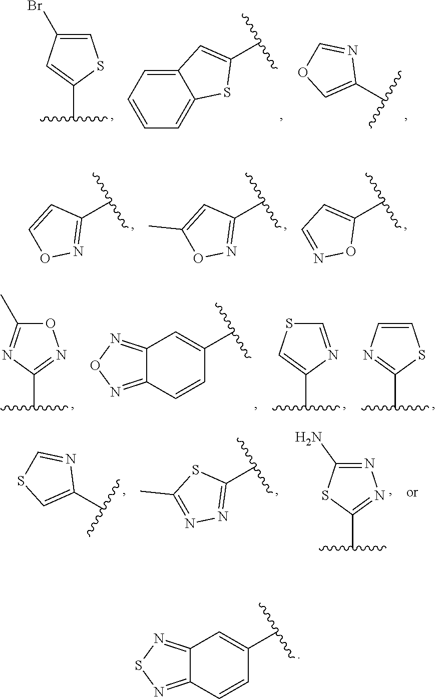

In some embodiments, R¹ is

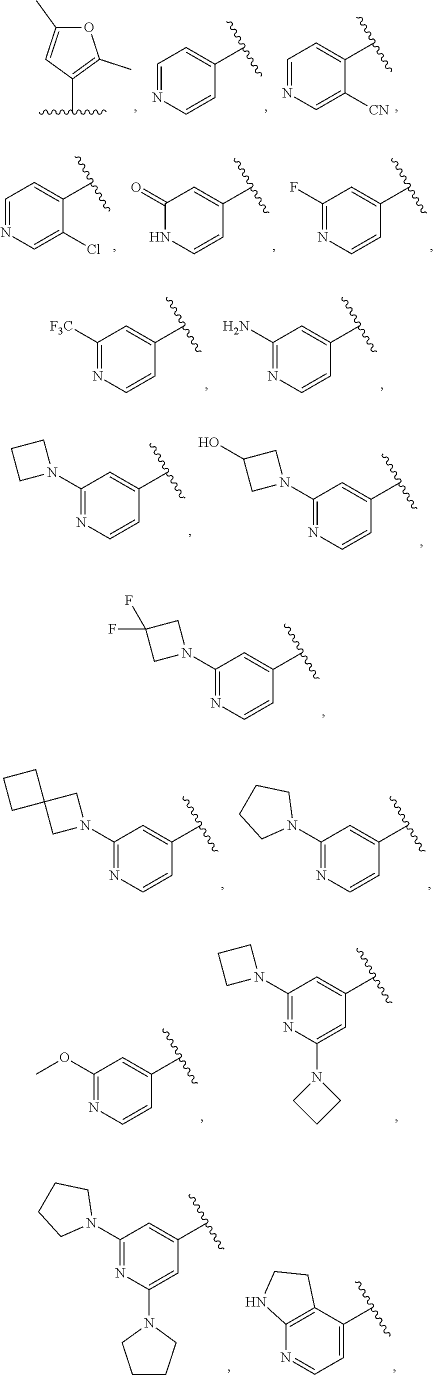

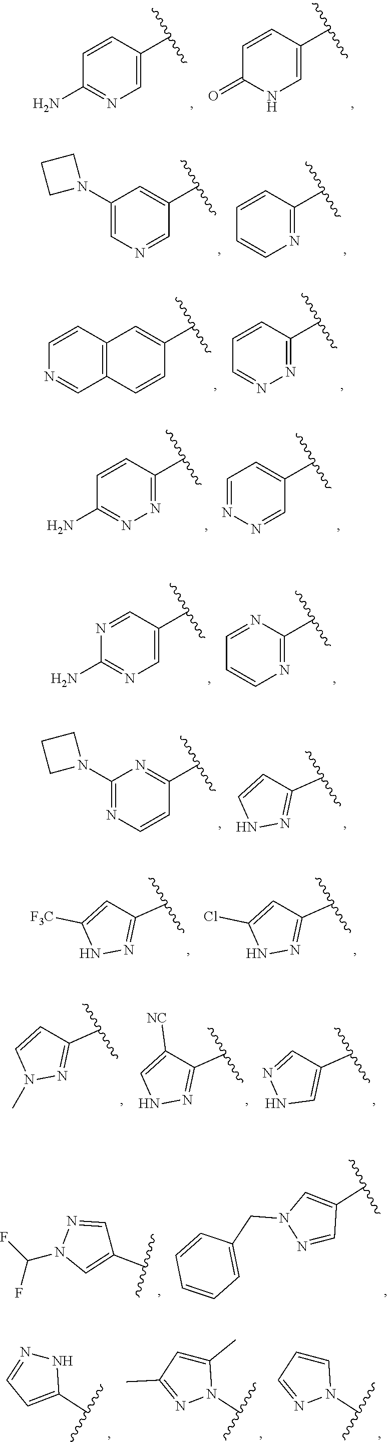

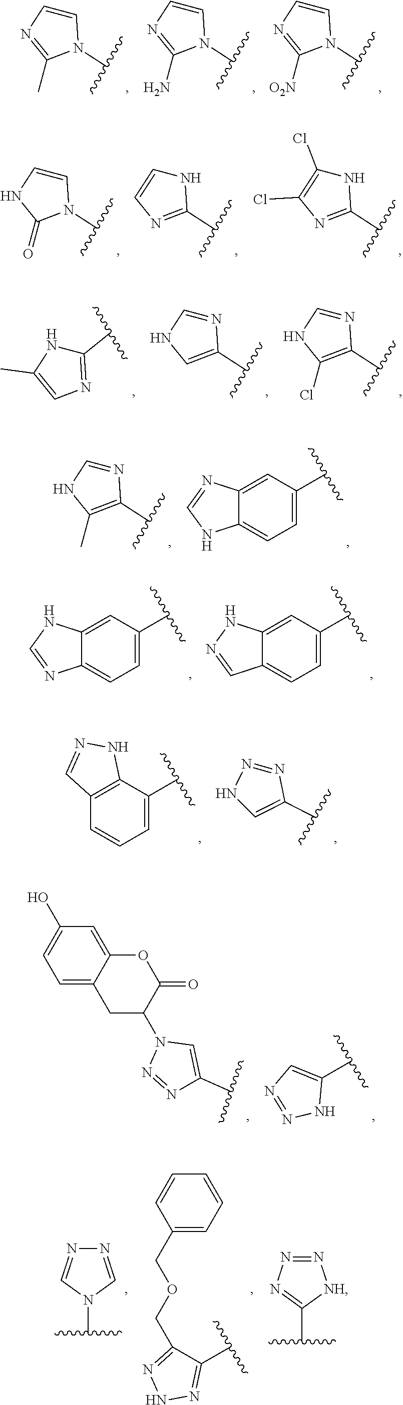

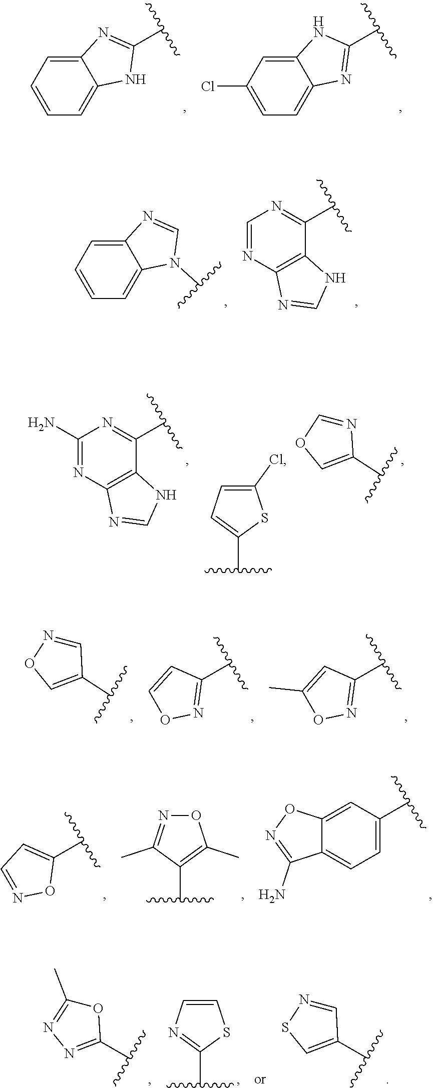

In some embodiments, R¹ is

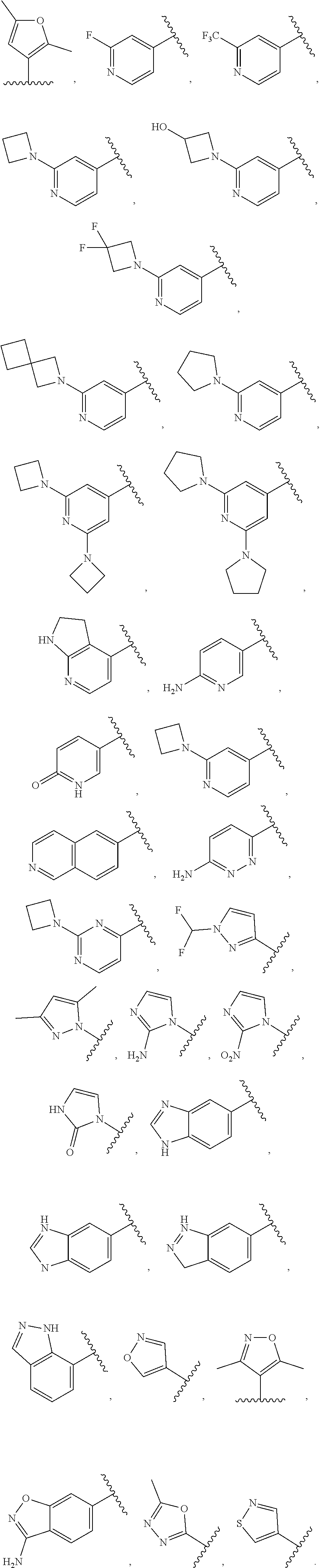

In some embodiments, R¹ is

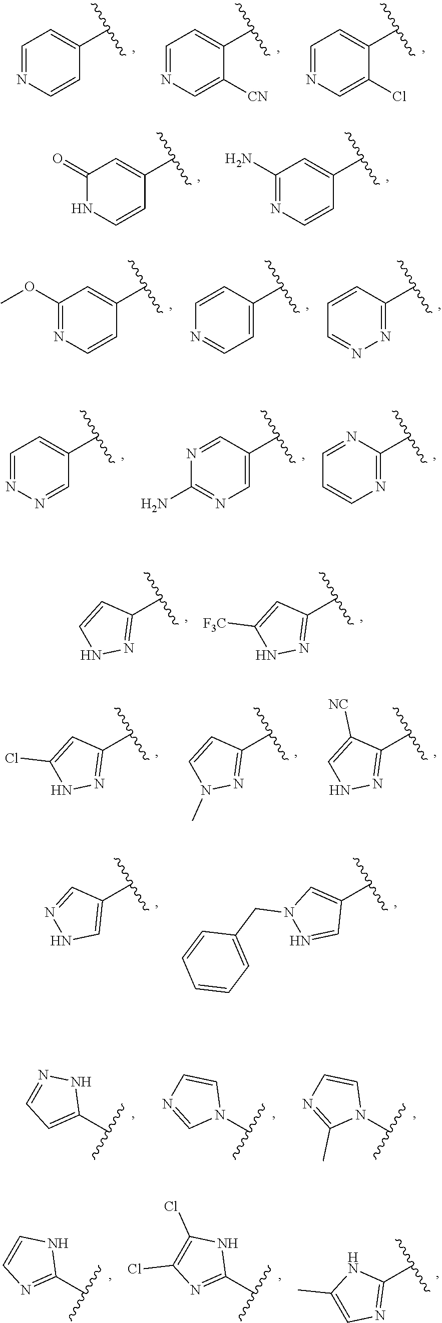

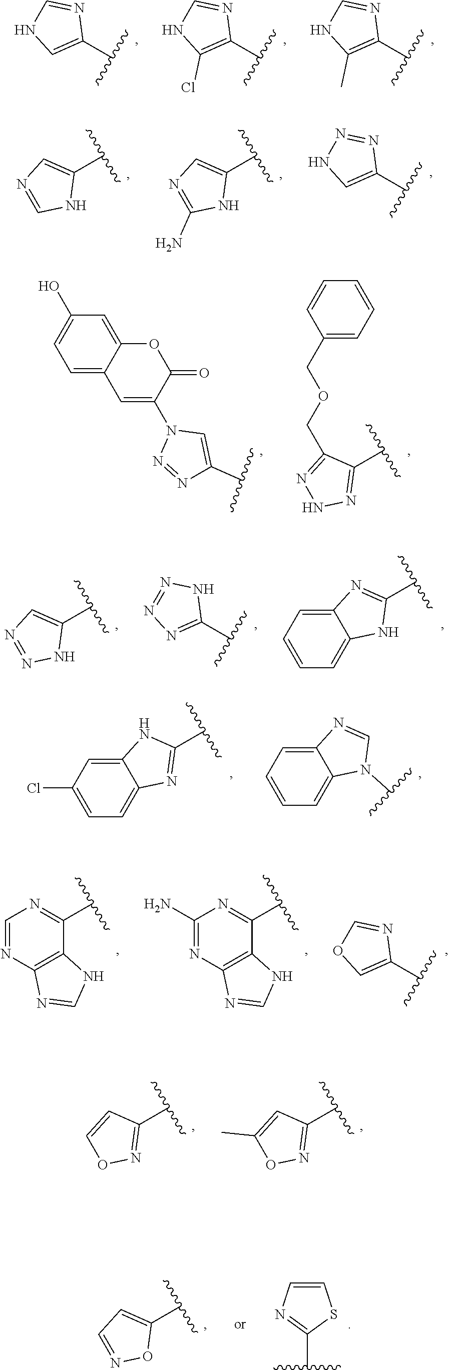

In some embodiments, R¹ is

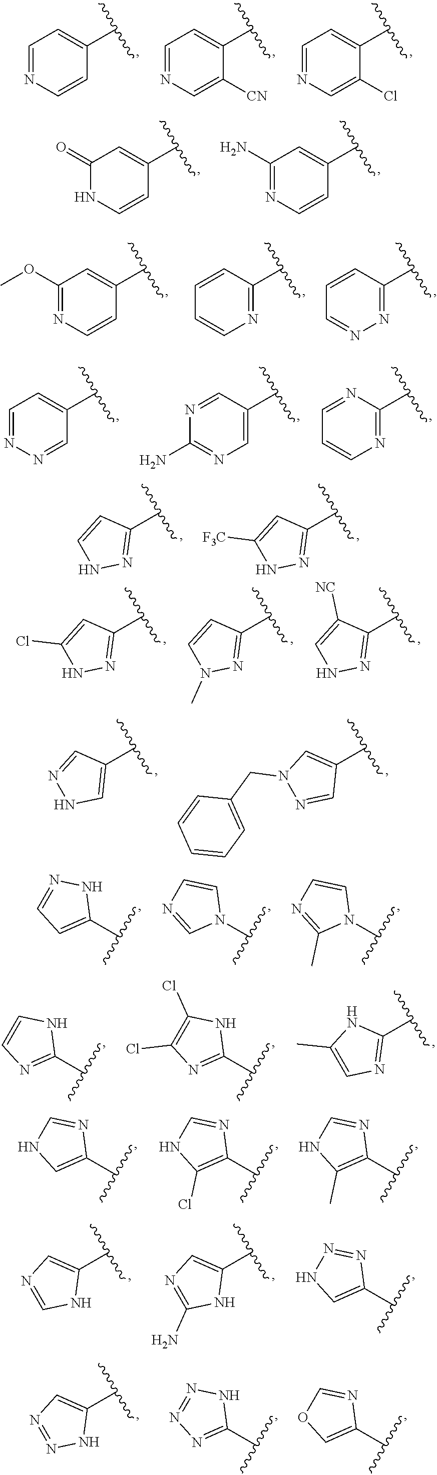

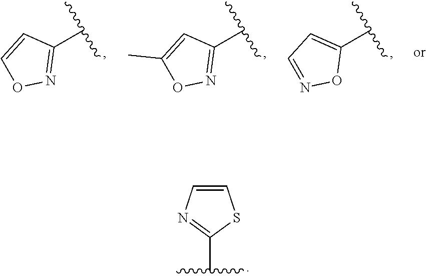

In some embodiments, R¹ is

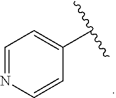 [SMILES: *c1ccncc1]

In some embodiments, R¹ is

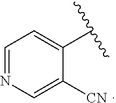 [SMILES: *c1ccncc1C#N]

In some embodiments, R¹ is

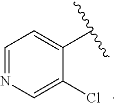 [SMILES: *c1ccncc1Cl]

In some embodiments, R¹ is

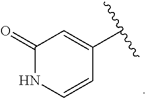 [SMILES: *c1cc[nH]c(=O)c1]

In some embodiments, R¹ is

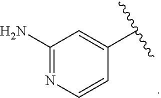 [SMILES: *c1ccnc(N)c1]

In some embodiments, R¹ is

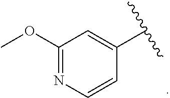 [SMILES: *c1ccnc(OC)c1]

In some embodiments, R¹ is

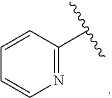 [SMILES: *c1ccccn1]

In some embodiments, R¹ is

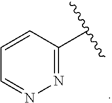 [SMILES: *c1cccnn1]

In some embodiments, R¹ is

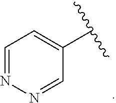 [SMILES: *c1ccnnc1]

In some embodiments, R¹ is

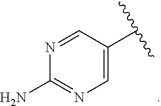 [SMILES: *c1cnc(N)nc1]

In some embodiments, R¹ is

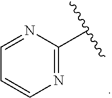 [SMILES: *c1ncccn1]

In some embodiments, R¹ is

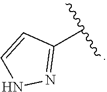 [SMILES: *c1cc[nH]n1]

In some embodiments, R¹ is

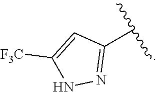 [SMILES: *c1cc(C(F)(F)F)[nH]n1]

In some embodiments, R¹ is

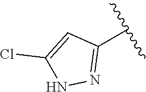 [SMILES: *c1cc(Cl)[nH]n1]

In some embodiments, R¹ is

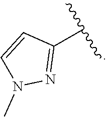 [SMILES: *c1ccn(C)n1]

In some embodiments, R¹ is

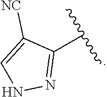 [SMILES: *c1n[nH]cc1C#N]

In some embodiments, R¹ is

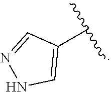 [SMILES: *c1cn[nH]c1]

In some embodiments, R¹ is

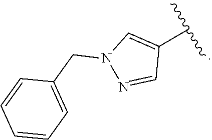 [SMILES: *c1cnn(Cc2ccccc2)c1]

In some embodiments, R¹ is

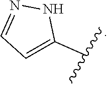 [SMILES: *c1ccn[nH]1]

In some embodiments, R¹ is

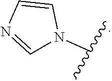 [SMILES: *n1ccnc1]

In some embodiments, R¹ is

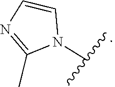 [SMILES: *n1ccnc1C]

In some embodiments, R¹ is

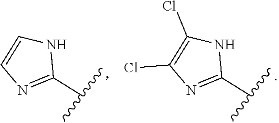 [SMILES: *c1ncc[nH]1 ; *c1nc(Cl)c(Cl)[nH]1]

In some embodiments, R¹ is

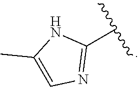 [SMILES: *c1ncc(C)[nH]1]

In some embodiments, R¹ is

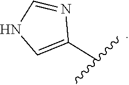 [SMILES: *c1c[nH]cn1]

In some embodiments, R¹ is

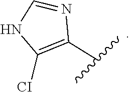

In some embodiments, R¹ is

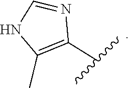 [SMILES: *c1nc[nH]c1C]

In some embodiments, R¹ is

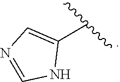 [SMILES: *c1cnc[nH]1]

In some embodiments, R¹ is

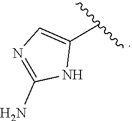 [SMILES: *c1cnc(N)[nH]1]

In some embodiments, R¹ is

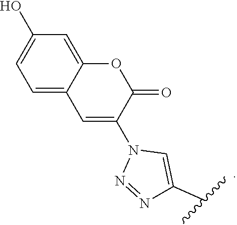 [SMILES: *c1cn(-c2cc3ccc(O)cc3oc2=O)nn1]

In some embodiments, R¹ is

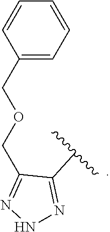 [SMILES: *c1n[nH]nc1COCc1ccccc1]

In some embodiments, R¹ is

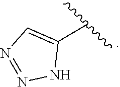 [SMILES: *c1cnn[nH]1]

In some embodiments, R¹ is

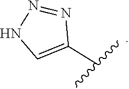 [SMILES: *c1c[nH]nn1]

In some embodiments, R¹ is

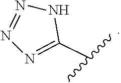 [SMILES: *c1nnn[nH]1]

In some embodiments, R¹ is

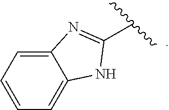 [SMILES: *c1nc2ccccc2[nH]1]

In some embodiments, R¹ is

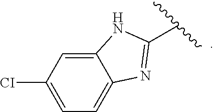

In some embodiments, R¹ is

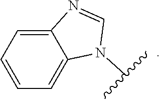 [SMILES: *n1cnc2ccccc21]

In some embodiments, R¹ is

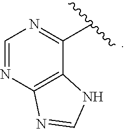 [SMILES: *c1ncnc2nc[nH]c12]

In some embodiments, R¹ is

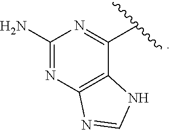 [SMILES: *c1nc(N)nc2nc[nH]c12]

In some embodiments, R¹ is

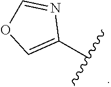 [SMILES: *c1cocn1]

In some embodiments, R¹ is

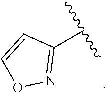 [SMILES: *c1ccon1]

In some embodiments, R¹ is

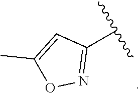 [SMILES: *c1cc(C)on1]

In some embodiments, R¹ is

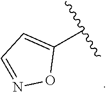 [SMILES: *c1ccno1]

In some embodiments, R¹ is

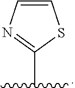 [SMILES: *c1nccs1]

In some embodiments, R¹ is —OR^{1a} or —N(R^{1a})₂. In some embodiments, R¹ is —OR^{1a}. In some embodiments, R¹ is —OH. In some embodiments, R¹ is —O (optionally substituted alkyl). In some embodiments, R¹ is —O (optionally substituted C₁₋₆ alkyl). In some embodiments, R¹ is —O (optionally substituted methyl), —O (optionally substituted ethyl), —O (optionally substituted n-propyl), —O (optionally substituted isopropyl), —O (optionally substituted n-butyl), —O (optionally substituted tert-butyl), —O (optionally substituted sec-butyl), —O (optionally substituted isobutyl), —O (optionally substituted n-pentyl), —O (optionally substituted 3-pentanyl), —O (optionally substituted amyl), —O (optionally substituted neopentyl), —O (optionally substituted 3-methyl-2-butanyl), —O (optionally substituted tert-amyl), or —O (optionally substituted n-hexyl). In some embodiments, R¹ is —O (optionally substituted isopropyl). In some embodiments, R¹ is —O (optionally substituted isobutyl).

In some embodiments, R¹ is —N(R^{1a})₂. In some embodiments, R¹ is —NR^{1a} (optionally substituted alkyl). In some embodiments, R¹ is —NR^{1a} (optionally substituted C₁₋₆ alkyl). In some embodiments, R¹ is —NR^{1a} (optionally substituted methyl), —NR^{1a} (optionally substituted ethyl), —NR^{1a} (optionally substituted n-propyl), —NR^{1a} (optionally substituted isopropyl), —NR^{1a} (optionally substituted n-butyl), —NR^{1a} (optionally substituted tert-butyl), —NR^{1a} (optionally substituted sec-butyl), —NR^{1a} (optionally substituted isobutyl), —NR^{1a} (optionally substituted n-pentyl), —NR^{1a} (optionally substituted 3-pentanyl), —NR^{1a} (optionally substituted amyl), —NR^{1a} (optionally substituted neopentyl), —NR^{1a} (optionally substituted 3-methyl-2-butanyl), —NR^{1a} (optionally substituted tert-amyl), or —NR^{1a} (optionally substituted n-hexyl). In some embodiments, R¹ is —NR^{1a} (optionally substituted methyl). In some embodiments, R¹ is —NR^{1a} (optionally substituted ethyl). In some embodiments, R¹ is —NR^{1a} (optionally substituted isopropyl). In some embodiments, R¹ is —NR^{1a} (optionally substituted n-butyl). In some embodiments, R¹ is —NR^{1a}(substituted methyl). In some embodiments, R¹ is —NR^{1a}(substituted ethyl). In some embodiments, R¹ is —NR^{1a}(substituted isopropyl). In some embodiments, R¹ is —NR^{1a}(substituted n-butyl). In some embodiments, R¹ is —NR^{1a} (unsubstituted methyl). In some embodiments, R¹ is —NR^{1a} (unsubstituted ethyl). In some embodiments, R¹ is —NR^{1a} (unsubstituted isopropyl). In some embodiments, R¹ is —NR^{1a} (unsubstituted n-butyl). In some embodiments, R¹ is —N (optionally substituted alkyl)₂. In some embodiments, R¹ is —N (optionally substituted C₁₋₆ alkyl)₂.

In some embodiments, R¹ is —NR^{1a} (alkyl substituted with ═O), —NR^{1a} (alkyl substituted with —OR^{1a}), —NR^{1a} (alkyl substituted with optionally substituted carbocyclyl), —NR^{1a} (alkyl substituted with optionally substituted aryl), or —NR^{1a} (alkyl substituted with optionally substituted heteroaryl). In some embodiments, R¹ is —NR^{1a} (alkyl substituted with ═O). In some embodiments, R¹ is —NR^{1a} (C₁₋₆ alkyl substituted with ═O). In some embodiments, R¹ is —NR^{1a} (methyl substituted with ═O).

In some embodiments, R¹ is —NR^{1a} (alkyl substituted with —OR^{1a}). In some embodiments, R¹ is —NR^{1a} (C₁₋₆ alkyl substituted with —OR^{1a}). In some embodiments, R¹ is —NR^{1a} (ethyl substituted with —OR^{1a}). In some embodiments, R¹ is —NR^{1a} (alkyl substituted with —O (optionally substituted C₁₋₆ alkyl)). In some embodiments, R¹ is —NR^{1a} (C₁₋₆ alkyl substituted with —O (optionally substituted C₁₋₆ alkyl)). In some embodiments, R¹ is —NR^{1a} (ethyl substituted with —O (optionally substituted C₁₋₆ alkyl)). In some embodiments, R¹ is —NR^{1a} (C₁₋₆ alkyl substituted with —O (unsubstituted C₁₋₆ alkyl)). In some embodiments, R¹ is —NR^{1a} (alkyl substituted with —OCH₃). In some embodiments, R¹ is —NR^{1a} (C₁₋₆ alkyl substituted with —OCH₃). In some embodiments, R¹ is —NR^{1a} (ethyl substituted with —OCH₃).

In some embodiments, R¹ is —NR^{1a} (alkyl substituted with optionally substituted carbocyclyl). In some embodiments, R¹ is —NR^{1a} (alkyl substituted with optionally substituted C₃₋₆ carbocyclyl). In some embodiments, R¹ is —NR^{1a} (alkyl substituted with substituted C₃₋₆ carbocyclyl). In some embodiments, R¹ is —NR^{1a} (alkyl substituted with unsubstituted C₃₋₆ carbocyclyl). In some embodiments, R¹ is —NR^{1a} (alkyl substituted with ═O and optionally substituted C₃₋₆ carbocyclyl). In some embodiments, R¹ is —NR^{1a} (methyl substituted with ═O and optionally substituted C₃₋₆ carbocyclyl).

In some embodiments, R¹ is —NR^{1a} (alkyl substituted with optionally substituted aryl). In some embodiments, R¹ is —NR^{1a} (alkyl substituted with optionally substituted phenyl). In some embodiments, R¹ is —NR^{1a} (alkyl substituted with unsubstituted phenyl). In some embodiments, R¹ is —NR^{1a} (C₁₋₆ alkyl substituted with optionally substituted phenyl). In some embodiments, R¹ is —NR^{1a} (C₁₋₆ alkyl substituted with unsubstituted phenyl). In some embodiments, R¹ is —NR^{1a} (ethyl substituted with unsubstituted phenyl).

In some embodiments, R¹ is —NR^{1a} (alkyl substituted with optionally substituted heteroaryl). In some embodiments, R¹ is —NR^{1a} (alkyl substituted with optionally substituted imidazolyl) or —NR^{1a} (alkyl substituted with optionally substituted pyridyl). In some embodiments, R¹ is —NR^{1a} (C₁₋₆ alkyl substituted with optionally substituted imidazolyl) or —NR^{1a} (C₁₋₆ alkyl substituted with optionally substituted pyridyl). In some embodiments, R¹ is —NR^{1a} (C₁₋₆ alkyl substituted with unsubstituted imidazolyl) or —NR^{1a} (C₁₋₆ alkyl substituted with unsubstituted pyridyl). In some embodiments, R¹ is —NR^{1a} (ethyl substituted with unsubstituted imidazolyl) or —NR^{1a} (ethyl substituted with unsubstituted pyridyl).

In some embodiments, R¹ is —NHR^{1a}. In some embodiments, R¹ is —NH (optionally substituted alkyl). In some embodiments, R¹ is —NH (optionally substituted C₁₋₆ alkyl). In some embodiments, R¹ is —NH₂.

In some embodiments, R¹ is —OH, —OCHF₂, —OCF₃,

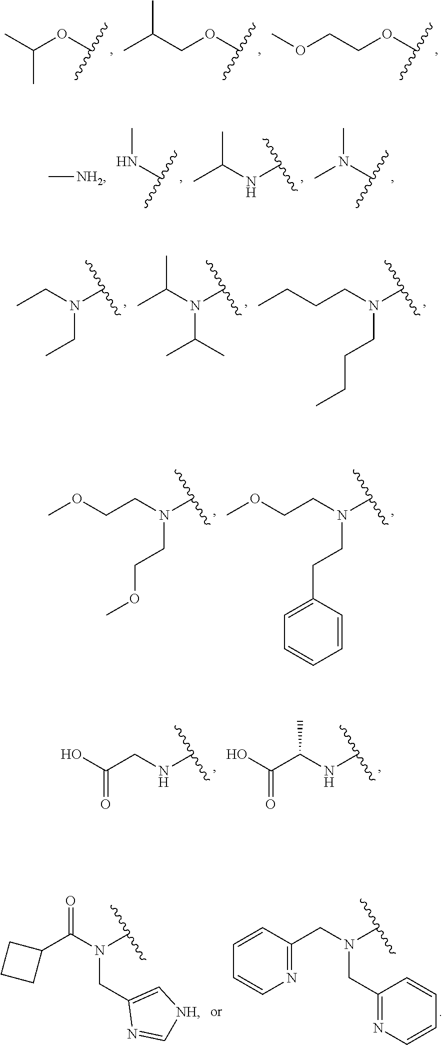

In some embodiments, R¹ is —OH,

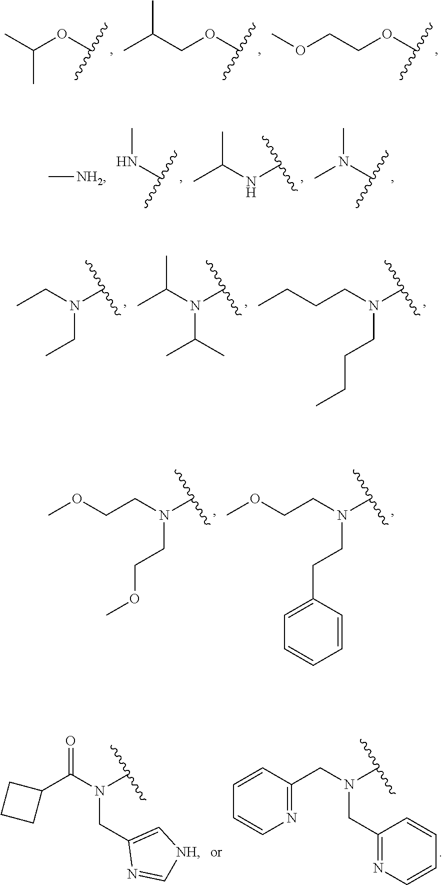

In some embodiments, R¹ is —OH, —OCHF₂, —OCF₃,

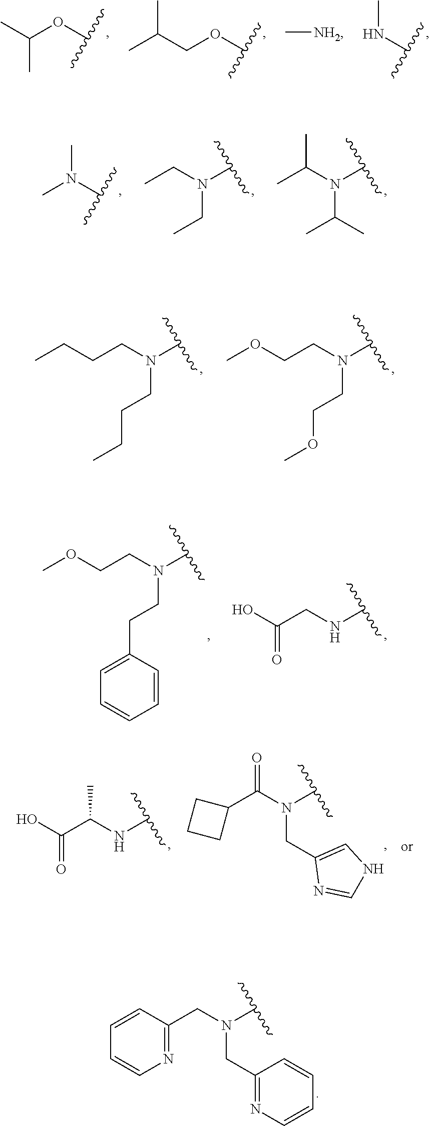

In some embodiments, R¹ is —OCHF₂, —OCF₃,

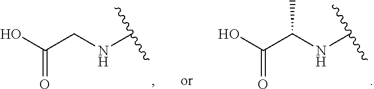

In some embodiments, R¹ is —OH,

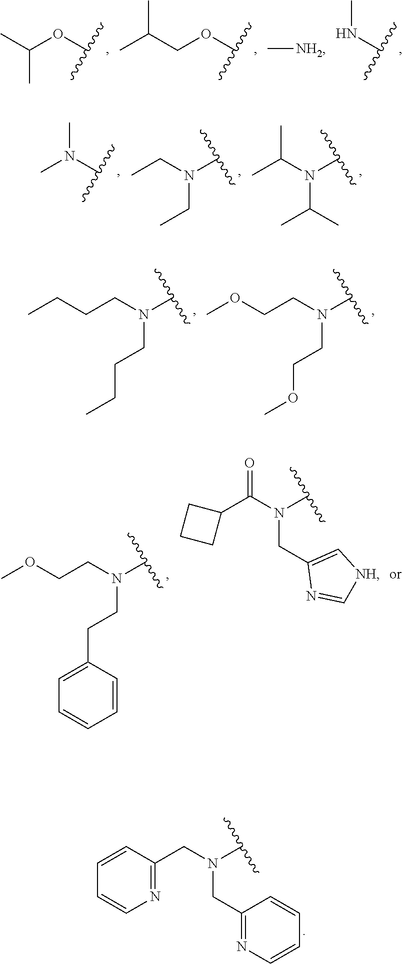

In some embodiments, R¹ is —OH,

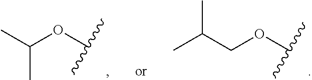

In some embodiments, R¹ is —NH₂,

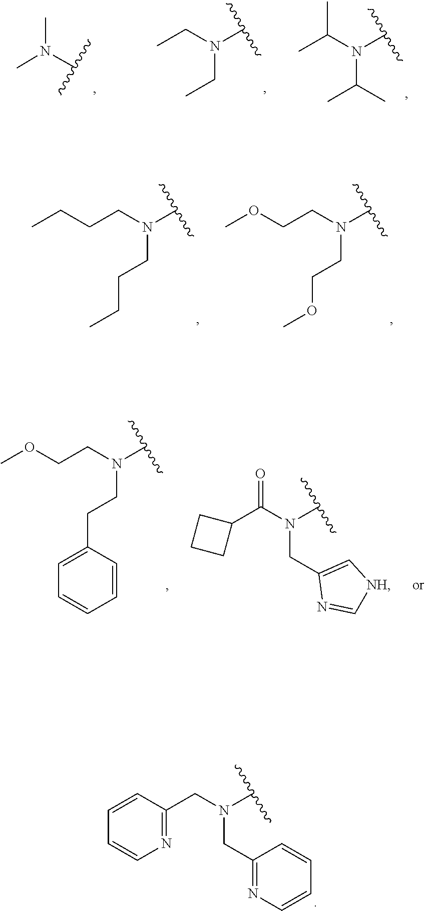

As generally described herein, X is —CH(R²)— or —NH—. In some embodiments, X is —CH(R²)—. In some embodiments, X is —CH₂—. In some embodiments, X is —CH (optionally substituted alkyl)-. In some embodiments, X is —CH (substituted alkyl)-. In some embodiments, X is —CH (unsubstituted alkyl)-. In some embodiments, X is —CH (optionally substituted C₁₋₆ alkyl)-. In some embodiments, X is —CH (substituted C₁₋₆ alkyl)-. In some embodiments, X is —CH (unsubstituted C₁₋₆ alkyl)-. In some embodiments, X is —CH(CH₃)—. In some embodiments, X is —CH(R²)—, and R¹ and R² are joined together with their intervening atoms to form optionally substituted carbocyclyl or optionally substituted heterocyclyl. In some embodiments, X is —CH(R²)—, and R¹ and R² are joined together with their intervening atoms to form optionally substituted carbocyclyl. In some embodiments, X is —CH(R²)—, and R¹ and R² are joined together with their intervening atoms to form optionally substituted heterocyclyl. In some embodiments, X is —NH—.

As generally described herein, R² is hydrogen or optionally substituted alkyl. In some embodiments, R² is hydrogen. In some embodiments, R² is optionally substituted alkyl. In some embodiments, R² is optionally substituted C₁₋₁₂ alkyl. In some embodiments, R² is optionally substituted C₁₋₆ alkyl. In some embodiments, R² is optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In some embodiments, R² is substituted C₁₋₆ alkyl. In some embodiments, R² is substituted methyl, substituted ethyl, substituted n-propyl, substituted isopropyl, substituted n-butyl, substituted tert-butyl, substituted sec-butyl, substituted isobutyl, substituted n-pentyl, substituted 3-pentanyl, substituted amyl, substituted neopentyl, substituted 3-methyl-2-butanyl, substituted tert-amyl, or substituted n-hexyl. In some embodiments, R² is unsubstituted C₁₋₆ alkyl. In some embodiments, R² is methyl, ethyl, n-propyl, isopropyl, n-butyl, tert-butyl, sec-butyl, isobutyl, n-pentyl, 3-pentanyl, amyl, neopentyl, 3-methyl-2-butanyl, tert-amyl, or n-hexyl. In some embodiments, R² is optionally substituted methyl. In some embodiments, R² is substituted methyl. In some embodiments, R² is methyl.

As generally described herein, n is 0, 1, 2, 3, 4, or 5. In some embodiments, n is 0 or 1. In some embodiments, n is 0. In some embodiments, n is 1. In some embodiments, n is 2. In some embodiments, n is 3. In some embodiments, n is 4. In some embodiments, n is 5. In some embodiments, X is —CH₂—, and n is 0.

As generally described herein, each instance of R^{Y} is independently hydrogen or halogen, or two instances of R^{Y} are taken together to form ═O. In some embodiments, each instance of R^{Y} is independently hydrogen or halogen. In some embodiments, at least one instance of R^{Y} is hydrogen. In some embodiments, at least one instance of R^{Y} is halogen. In some embodiments, at least one instance of R^{Y} is —F. In some embodiments, each instance of R^{Y} is hydrogen. In some embodiments, each instance of R^{Y} is halogen. In some embodiments, each instance of R^{Y} is —F. In some embodiments, two instances of R^{Y} are taken together to form ═O.

In some embodiments, each instance of Y is independently —C(R^{Y})₂—, —O—, or —N(R^{1a})—. In some embodiments, at least one instance of Y is —C(R^{Y})₂—. In some embodiments, at least one instance of Y is —CHR^{Y}—. In some embodiments, at least one instance of Y is —CH(CH₃)—. In some embodiments, at least one instance of Y is —CH₂—. In some embodiments, at least one instance of Y is —CFR^{Y}—. In some embodiments, at least one instance of Y is —CHF—. In some embodiments, at least one instance of Y is —CF₂—. In some embodiments, at least one instance of Y is —C(═O)—. In some embodiments, at least one instance of Y is —O— or —N(R^{1a})—. In some embodiments, at least one instance of Y is —O—. In some embodiments, at least one instance of Y is —N(R^{1a})—. In some embodiments, at least one instance of Y is —NH—. In some embodiments, at least one instance of Y is —N(R^{1a})—, and R^{1a} is optionally substituted alkyl or nitrogen protecting group. In some embodiments, at least one instance of Y is —N(R^{1a})—, and R^{1a} is optionally substituted C₁₋₆ alkyl. In some embodiments, at least one instance of Y is —N(R^{1a})—, and R^{1a} is nitrogen protecting group.

In some embodiments, (Y)ₙ is —CH₂—. In some embodiments, X is —CH₂—, and (Y)ₙ is —CH₂—. In some embodiments, (Y)ₙ is —(CH₂)₂—. In some embodiments, X is —CH₂—, and (Y)ₙ is —(CH₂)₂—. In some embodiments, (Y)ₙ is —OCH₂—. In some embodiments, X is —CH₂—, and (Y)ₙ is —OCH₂—. In some embodiments, (Y)ₙ is —CF₂—. In some embodiments, X is —CH₂—, and (Y)ₙ is —CF₂—. In some embodiments, (Y)ₙ is —C(═O)—. In some embodiments, X is —CH₂—, and (Y)ₙ is —C(═O)—. In some embodiments, (Y)ₙ is —CH₂C(═O)— or —C(═O)CH₂—. In some embodiments, X is —CH₂—, and (Y)ₙ is —CH₂C(═O)— or —C(═O)CH₂—. In some embodiments, (Y)ₙ is —NH—. In some embodiments, X is —CH₂—, and (Y)ₙ is —NH—. In some embodiments, (Y)ₙ is —NHCH₂— or —CH₂NH—. In some embodiments, X is —CH₂—, and (Y)ₙ is —NHCH₂— or —CH₂NH—. In some embodiments, (Y)ₙ is —C(═O)NHCH₂— or —CH₂NHC(═O)—. In some embodiments, X is —CH₂—, and (Y)ₙ is —C(═O)NHCH₂— or —CH₂NHC(═O)—.

In some embodiments, R¹ and R² are joined together with their intervening atoms to form optionally substituted carbocyclyl or optionally substituted heterocyclyl. In some embodiments, R¹ and R² are joined together with their intervening atoms to form optionally substituted carbocyclyl. In some embodiments, R¹ and R² are joined together with their intervening atoms to form optionally substituted C₃₋₁₀ carbocyclyl. In some embodiments, R¹ and R² are joined together with their intervening atoms to form optionally substituted C₃₋₆ carbocyclyl. In some embodiments, R¹ and R² are joined together with their intervening atoms to form optionally substituted cyclobutyl, optionally substituted cyclohexyl, optionally substituted spiro[2.2]pentyl, optionally substituted spiro[2.3]hexyl, or optionally substituted spiro[3.3]heptyl. In some embodiments, R¹ and R² are joined together with their intervening atoms to form optionally substituted cyclobutyl. In some embodiments, R¹ and R² are joined together with their intervening atoms to form substituted cyclobutyl. In some embodiments, R¹ and R² are joined together with their intervening atoms to form unsubstituted cyclobutyl.

In some embodiments, R¹ and R² are joined together with their intervening atoms to form carbocyclyl substituted with halogen, optionally substituted alkyl, —OR^{1a}, and/or —N(R^{1a})₂. In some embodiments, R¹ and R² are joined together with their intervening atoms to form carbocyclyl substituted with halogen. In some embodiments, R¹ and R² are joined together with their intervening atoms to form carbocyclyl substituted with —F, —Cl, or —Br. In some embodiments, R¹ and R² are joined together with their intervening atoms to form carbocyclyl substituted with —F. In some embodiments, R¹ and R² are joined together with their intervening atoms to form cyclobutyl substituted with halogen. In some embodiments, R¹ and R² are joined together with their intervening atoms to form cyclobutyl substituted with —F. In some embodiments, R¹ and R² are joined together with their intervening atoms to form carbocyclyl substituted with —N(R^{1a})₂. In some embodiments, R¹ and R² are joined together with their intervening atoms to form cyclobutyl substituted with —N(R^{1a})₂. In some embodiments, R¹ and R² are joined together with their intervening atoms to form carbocyclyl substituted with —NHR^{1a}. In some embodiments, R¹ and R² are joined together with their intervening atoms to form cyclobutyl substituted with —NHR^{1a}. In some embodiments, R¹ and R² are joined together with their intervening atoms to form cyclobutyl substituted with —NH₂. In some embodiments, R¹ and R² are joined together with their intervening atoms to form carbocyclyl substituted with optionally substituted alkyl. In some embodiments, R¹ and R² are joined together with their intervening atoms to form carbocyclyl substituted with optionally substituted C₁₋₆ alkyl. In some embodiments, R¹ and R² are joined together with their intervening atoms to form carbocyclyl substituted with —OR^{1a}.

In some embodiments, R¹ and R² are joined together with their intervening atoms to form

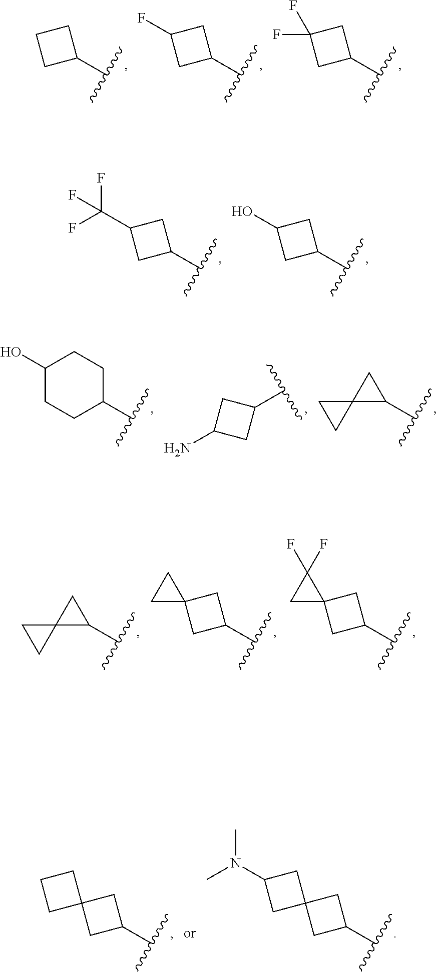

In some embodiments, R¹ and R² are joined together with their intervening atoms to form

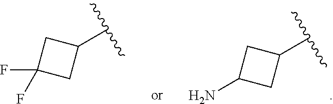 [SMILES: *C1CC(F)(F)C1 ; *C1CC(N)C1]

In some embodiments, R¹ and R² are joined together with their intervening atoms to form

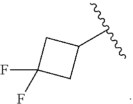 [SMILES: *C1CC(F)(F)C1]

In some embodiments, R¹ and R² are joined together with their intervening atoms to form

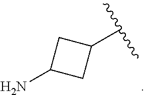 [SMILES: *C1CC(N)C1]

In some embodiments, R¹ and R² are joined together with their intervening atoms to form optionally substituted heterocyclyl. In some embodiments, R¹ and R² are joined together with their intervening atoms to form substituted 3-14 membered heterocyclyl. In some embodiments, R¹ and R² are joined together with their intervening atoms to form optionally substituted oxetanyl, optionally substituted tetrahydrofuranyl, optionally substituted tetrahydropyranyl, optionally substituted azetidinyl, optionally substituted pyrrolidinyl, optionally substituted 2-azaspiro[3.3]heptyl, optionally substituted 2,6-diazaspiro[3.4]octanyl, optionally substituted tetrahydrothiophenyl, optionally substituted dithiolanyl, or optionally substituted 1-imino-1-oxo-hexahydro-1λ⁶-thiopyranyl. In some embodiments, R¹ and R² are joined together with their intervening atoms to form optionally substituted oxetanyl. In some embodiments, R¹ and R² are joined together with their intervening atoms to form optionally substituted tetrahydrofuranyl. In some embodiments, R¹ and R² are joined together with their intervening atoms to form optionally substituted tetrahydropyranyl. In some embodiments, R¹ and R² are joined together with their intervening atoms to form optionally substituted azetidinyl. In some embodiments, R¹ and R² are joined together with their intervening atoms to form optionally substituted pyrrolidinyl. In some embodiments, R¹ and R² are joined together with their intervening atoms to form optionally substituted 2-azaspiro[3.3]heptyl. In some embodiments, R¹ and R² are joined together with their intervening atoms to form optionally substituted 2,6-diazaspiro[3.4]octanyl. In some embodiments, R¹ and R² are joined together with their intervening atoms to form optionally substituted tetrahydrothiophenyl. In some embodiments, R¹ and R² are joined together with their intervening atoms to form optionally substituted dithiolanyl. In some embodiments, R¹ and R² are joined together with their intervening atoms to form optionally substituted 1-imino-1-oxo-hexahydro-1λ⁶-thiopyranyl. In some embodiments, R¹ and R² are joined together with their intervening atoms to form optionally substituted azetidinyl or optionally substituted pyrrolidinyl. In some embodiments, R¹ and R² are joined together with their intervening atoms to form substituted azetidinyl or substituted pyrrolidinyl. In some embodiments, R¹ and R² are joined together with their intervening atoms to form substituted azetidinyl. In some embodiments, R¹ and R² are joined together with their intervening atoms to form substituted pyrrolidinyl. In some embodiments, R¹ and R² are joined together with their intervening atoms to form unsubstituted azetidinyl or unsubstituted pyrrolidinyl. In some embodiments, R¹ and R² are joined together with their intervening atoms to form unsubstituted azetidinyl. In some embodiments, R¹ and R² are joined together with their intervening atoms to form unsubstituted pyrrolidinyl.

In some embodiments, R¹ and R² are joined together with their intervening atoms to form optionally substituted heterocyclyl substituted with optionally substituted alkyl or ═O. In some embodiments, R¹ and R² are joined together with their intervening atoms to form 3-14 membered heterocyclyl substituted with optionally substituted alkyl or ═O. In some embodiments, R¹ and R² are joined together with their intervening atoms to form 3-14 membered heterocyclyl substituted with optionally substituted C₁₋₆ alkyl. In some embodiments, R¹ and R² are joined together with their intervening atoms to form 3-14 membered heterocyclyl substituted with ═O. In some embodiments, R¹ and R² are joined together with their intervening atoms to form 3-14 membered heterocyclyl substituted with optionally substituted C₁₋₆ alkyl and ═O.

In some embodiments, R¹ and R² are joined together with their intervening atoms to form

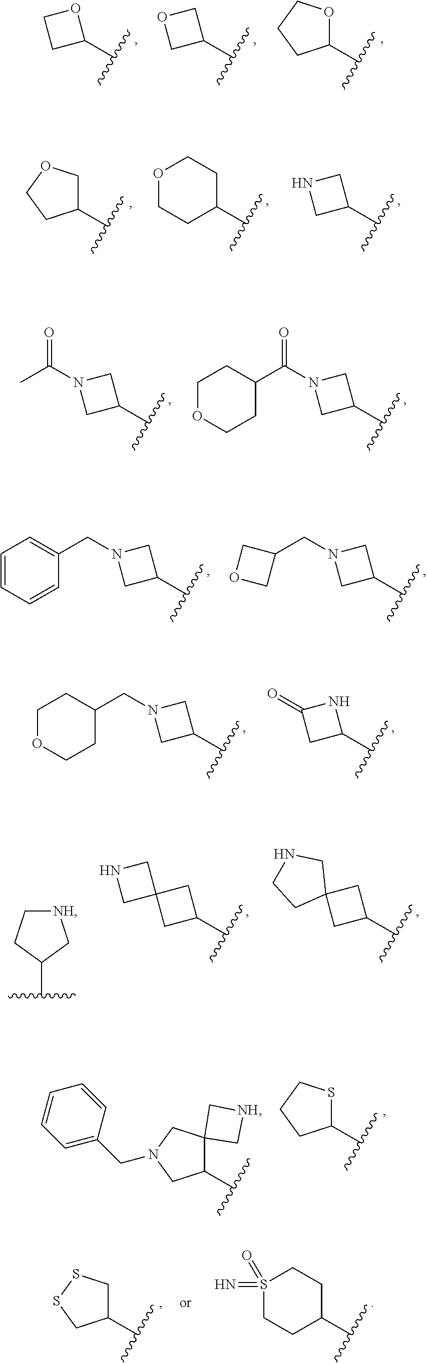

In some embodiments, R¹ and R² are joined together with their intervening atoms to form

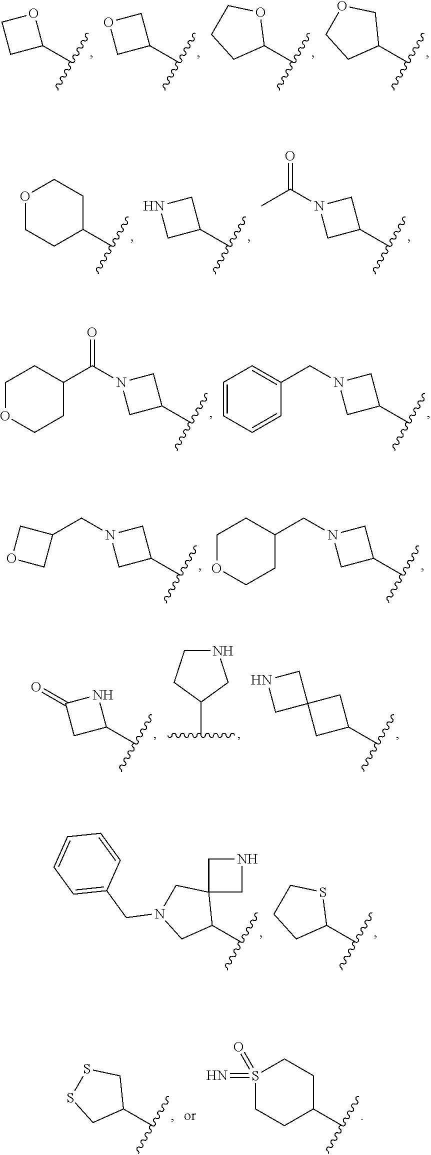

In some embodiments, R¹ and R² are joined together with their intervening atoms to form

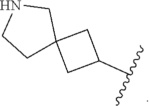 [SMILES: *C1CC2(CCNC2)C1]

In some embodiments, R¹ and R² are joined together with their intervening atoms to form

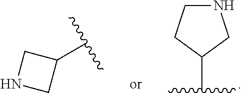 [SMILES: *C1CNC1 ; *C1CCNC1]

In some embodiments, R¹ and R² are joined together with their intervening atoms to form

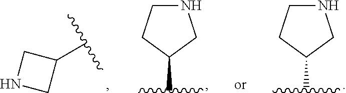 [SMILES: *C1CNC1 ; *[C@H]1CCNC1 ; *[C@@H]1CCNC1]

In some embodiments, R¹ and R² are joined together with their intervening atoms to form

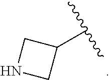 [SMILES: *C1CNC1]

In some embodiments, R¹ and R² are joined together with their intervening atoms to form

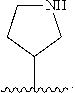 [SMILES: *C1CCNC1]

In some embodiments, R¹ and R² are joined together with their intervening atoms to form

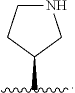 [SMILES: *[C@H]1CCNC1]

In some embodiments, R¹ and R² are joined together with their intervening atoms to form

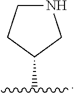 [SMILES: *[C@@H]1CCNC1]

As generally described herein, each instance of R^{1a} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or two instances of R^{1a} are joined together with their intervening atom to form an optionally substituted heterocyclic ring or optionally substituted heteroaryl ring.

In some embodiments, at least one instance of R^{1a} is hydrogen. In some embodiments, at least one instance of R^{1a} is optionally substituted acyl. In some embodiments, at least one instance of R^{1a} is optionally substituted alkyl. In some embodiments, at least one instance of R^{1a} is optionally substituted C₁₋₁₂ alkyl. In some embodiments, at least one instance of R^{1a} is optionally substituted C₁₋₆ alkyl. In some embodiments, at least one instance of R^{1a} is hydrogen, methyl, ethyl, isopropyl, n-butyl, isobutyl,

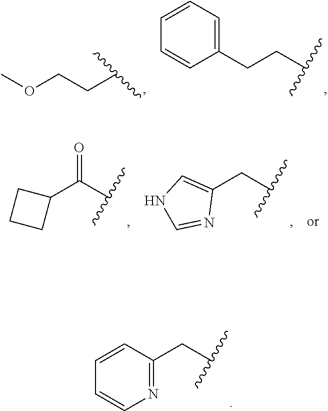

In some embodiments, at least one instance of R^{1a} is hydrogen.

In some embodiments, at least one instance of R^{1a} is optionally substituted C₂₋₁₂ alkenyl. In some embodiments, at least one instance of R^{1a} is optionally substituted C₂₋₆ alkenyl. In some embodiments, at least one instance of R^{1a} is optionally substituted C₂₋₁₂ alkynyl. In some embodiments, at least one instance of R^{1a} is optionally substituted C₂₋₆ alkynyl. In some embodiments, at least one instance of R^{1a} is optionally substituted heteroC₁₋₁₂ alkyl. In some embodiments, at least one instance of R^{1a} is optionally substituted heteroC₁₋₆ alkyl. In some embodiments, at least one instance of R^{1a} is optionally substituted heteroC₁₋₁₂ alkenyl. In some embodiments, at least one instance of R^{1a} is optionally substituted heteroC₁₋₆ alkenyl. In some embodiments, at least one instance of R^{1a} is optionally substituted heteroC₁₋₁₂ alkynyl. In some embodiments, at least one instance of R^{1a} is optionally substituted heteroC₁₋₆ alkynyl. In some embodiments, at least one instance of R^{1a} is optionally substituted C₃₋₁₄ cycloalkyl. In some embodiments, at least one instance of R^{1a} is optionally substituted 5-10 membered heterocyclyl. In some embodiments, at least one instance of R^{1a} is optionally substituted 6-14 membered aryl. In some embodiments, at least one instance of R^{1a} is optionally substituted 5-14 membered heteroaryl. In some embodiments, at least one instance of R^{1a} is a nitrogen protecting group when attached to a nitrogen atom. In some embodiments, at least one instance of R^{1a} is an oxygen protecting group when attached to an oxygen atom. In some embodiments, at least two instances of R^{1a} are joined together with their intervening atom to form an optionally substituted 5-10 membered heterocyclic ring. In some embodiments, at least two instances of R^{1a} are joined together with their intervening atom to form an optionally substituted 5-14 membered heteroaryl ring.

### R³

As generally described herein, R³ is optionally substituted carbocyclyl, optionally substituted heterocyclyl, optionally substituted aryl, or optionally substituted heteroaryl. In some embodiments, R³ is optionally substituted carbocyclyl. In some embodiments, R³ is optionally substituted C₃₋₁₀ carbocyclyl. In some embodiments, R³ is optionally substituted C₃₋₆ carbocyclyl. In some embodiments, R³ is optionally substituted heterocyclyl. In some embodiments, R³ is optionally substituted 3-14 membered heterocyclyl.

In some embodiments, R³ is optionally substituted heteroaryl. In some embodiments, R³ is optionally substituted 4-10 membered heteroaryl. In some embodiments, R³ is

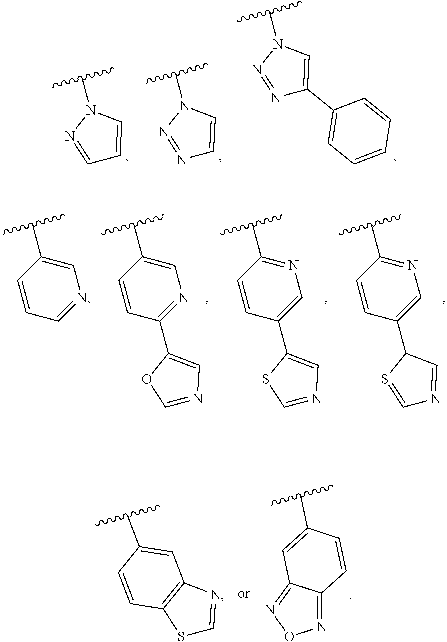

In some embodiments, R³ is optionally substituted aryl. In some embodiments, R³ is optionally substituted C₆₋₁₄ aryl. In some embodiments, R³ is optionally substituted phenyl. In some embodiments, R³ is unsubstituted phenyl. In some embodiments, R³ is substituted phenyl. In some embodiments, R³ is phenyl substituted with halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂. In some embodiments, R³ is phenyl substituted with halogen, optionally substituted alkyl, optionally substituted alkenyl, optionally substituted alkynyl, —CN, —OR^{1a}, optionally substituted aryl, and/or optionally substituted heteroaryl. In some embodiments, R³ is phenyl substituted with halogen, optionally substituted alkynyl, —CN, —OR^{1a}, optionally substituted phenyl, optionally substituted triazolyl, and/or optionally substituted tetrazolyl. In some embodiments, R³ is phenyl substituted with halogen, —OR¹a, and/or optionally substituted triazolyl.

In some embodiments, R³ is phenyl substituted with halogen, optionally substituted alkyl, optionally substituted alkenyl, optionally substituted alkynyl, —CN, —NO₂, —OR^{1a}, —N(R^{1a})₂, —NR^{1a}C(═O)R^{1a}, optionally substituted carbocyclyl, optionally substituted heterocyclyl, optionally substituted aryl, and/or optionally substituted heteroaryl. In some embodiments, R³ is phenyl substituted with halogen, optionally substituted C₁₋₃ alkyl, optionally substituted C₂₋₃ alkynyl, —CN, —NO₂, —OR^{1a}, —N(R^{1a})₂, —NR^{1a}C(═O)R^{1a}, optionally substituted C₃₋₄ carbocyclyl, optionally substituted C₃₋₄ heterocyclyl, optionally substituted phenyl, optionally substituted 5-6 membered heteroaryl containing 1, 2, 3, or 4 ring heteroatoms selected from N, O, and S, and/or optionally substituted 8-10 membered heteroaryl containing 1, 2, 3, or 4 ring heteroatoms selected from N, O, and S.

In some embodiments, R³ is phenyl substituted with halogen. In some embodiments, R³ is phenyl substituted with —F, —Cl, —Br, or —I. In some embodiments, R³ is phenyl substituted with —F or —Cl. In some embodiments, R³ is phenyl substituted with —F. In some embodiments, R³ is phenyl substituted with —Cl. In some embodiments, R³ is phenyl substituted with optionally substituted alkyl. In some embodiments, R³ is phenyl substituted with optionally substituted C₁₋₃ alkyl. In some embodiments, R³ is phenyl substituted with optionally substituted alkynyl. In some embodiments, R³ is phenyl substituted with optionally substituted C₂₆ alkynyl. In some embodiments, R³ is phenyl substituted with optionally substituted C₂₃ alkynyl. In some embodiments, R³ is phenyl substituted with —CN. In some embodiments, R³ is phenyl substituted with —NO₂. In some embodiments, R³ is phenyl substituted with —OR^{1a}. In some embodiments, R³ is phenyl substituted with —O (optionally substituted alkyl). In some embodiments, R³ is phenyl substituted with —O (optionally substituted C₁₋₆ alkyl). In some embodiments, R³ is phenyl substituted with —O(substituted C₁₋₆ alkyl). In some embodiments, R³ is phenyl substituted with —O (unsubstituted C₁₋₆ alkyl). In some embodiments, R³ is phenyl substituted with —O (optionally substituted C₁₋₃ alkyl). In some embodiments, R³ is phenyl substituted with —OCH₃. In some embodiments, R³ is phenyl substituted with —O (optionally substituted C₁₋₆ alkyl) and halogen. In some embodiments, R³ is phenyl substituted with —OCH₃ and —F or —Cl. In some embodiments, R³ is phenyl substituted with —N(R^{1a})₂. In some embodiments, R³ is phenyl substituted with —NR^{1a}C(═O)R^{1a}. In some embodiments, R³ is phenyl substituted with optionally substituted carbocyclyl. In some embodiments, R³ is phenyl substituted with optionally substituted C₃₋₄ carbocyclyl. In some embodiments, R³ is phenyl substituted with optionally substituted heterocyclyl. In some embodiments, R³ is phenyl substituted with optionally substituted C₃₋₄ heterocyclyl.

In some embodiments, R³ is phenyl substituted with optionally substituted phenyl. In some embodiments, R³ is phenyl substituted with substituted phenyl. In some embodiments, R³ is phenyl substituted with unsubstituted phenyl. In some embodiments, R³ is phenyl substituted with optionally substituted heteroaryl. In some embodiments, R³ is phenyl substituted with optionally substituted 5-6 membered heteroaryl containing 1, 2, 3, or 4 ring heteroatoms selected from N, O, and S. In some embodiments, R³ is phenyl substituted with optionally substituted 5-6 membered heteroaryl containing 1 ring S atom; 2 ring N atoms; 1 ring N atom and 1 ring 0 atom; 1 ring N atom and 1 ring S atom; 2 ring N atoms and 1 ring S atom; 3 ring N atoms; or 4 ring N atoms. In some embodiments, R³ is phenyl substituted with optionally substituted 8-10 membered heteroaryl containing 1, 2, 3, or 4 ring heteroatoms selected from N, O, and S. In some embodiments, R³ is phenyl substituted with optionally substituted 8-10 membered heteroaryl containing 2 ring N atoms; 1 ring N atom and 1 ring S atom; 2 ring N atoms and 1 ring S atom; 2 ring N atoms and 1 ring 0 atom; or 3 ring N atoms and 1 ring S atom.

In some embodiments, R³ is phenyl substituted with optionally substituted triazolyl. In some embodiments, R³ is phenyl substituted with substituted triazolyl. In some embodiments, R³ is phenyl substituted with triazolyl, wherein the triazolyl is substituted with halogen, optionally substituted alkyl, optionally substituted heterocyclyl, optionally substituted aryl, and/or optionally substituted heteroaryl. In some embodiments, R³ is phenyl substituted with triazolyl, wherein the triazolyl is substituted with —Cl, optionally substituted C₁₋₆ alkyl, optionally substituted 4-6 membered heterocyclyl, optionally substituted phenyl, or optionally substituted 5-6 membered heteroaryl. In some embodiments, R³ is phenyl substituted with triazolyl, wherein the triazolyl is substituted with —Cl. In some embodiments, R³ is phenyl substituted with triazolyl, wherein the triazolyl is substituted with substituted C₁₋₆ alkyl. In some embodiments, R³ is phenyl substituted with triazolyl, wherein the triazolyl is substituted with substituted methyl. In some embodiments, R³ is phenyl substituted with unsubstituted triazolyl. In some embodiments, R³ is phenyl substituted with optionally substituted tetrazolyl.

In some embodiments, R³ is

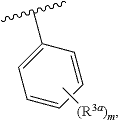

wherein:
each instance of R^{3a} is independently halogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted carbocyclyl, substituted or unsubstituted heterocyclyl, substituted or unsubstituted aryl, substituted or unsubstituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring; and
m is 0, 1, 2, 3, 4, or 5.

In some embodiments, at least one instance of R^{3a} is independently halogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted carbocyclyl, substituted or unsubstituted heterocyclyl, substituted or unsubstituted aryl, substituted or unsubstituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In some embodiments, at least one instance of R^{3a} is halogen, optionally substituted alkyl, optionally substituted alkenyl, optionally substituted alkynyl, —CN, —OR^{1a}, optionally substituted aryl, or optionally substituted heteroaryl. In some embodiments, at least one instance of R^{3a} is halogen, optionally substituted alkynyl, —CN, —OR^{1a}, optionally substituted phenyl, optionally substituted triazolyl, or optionally substituted tetrazolyl. In some embodiments, at least one instance of R^{3a} is halogen, —OR^{1a}, or optionally substituted triazolyl.

In some embodiments, at least one instance of R^{3a} is halogen, optionally substituted alkyl, optionally substituted alkenyl, optionally substituted alkynyl, —CN, —NO₂, —OR^{1a}, —N(R^{1a})₂, —NR^{1a}C(═O)R^{1a}, —C(═O)R^{1a}, optionally substituted carbocyclyl, optionally substituted heterocyclyl, optionally substituted aryl, or optionally substituted heteroaryl. In some embodiments, at least one instance of R^{3a} is halogen, optionally substituted C₁₋₃ alkyl, optionally substituted C₂₋₃ alkynyl, —CN, —NO₂, —OR^{1a}, —N(R^{1a})₂, —NR^{1a}C(═O)R^{1a}, —C(═O)R^{1a}, optionally substituted C₃₋₄ carbocyclyl, optionally substituted 3-4 membered heterocyclyl, optionally substituted phenyl, optionally substituted 5-6 membered heteroaryl containing 1, 2, 3, or 4 ring heteroatoms selected from N, O, and S, and/or optionally substituted 8-10 membered heteroaryl containing 1, 2, 3, or 4 ring heteroatoms selected from N, O, and S.

In some embodiments, at least one instance of R^{3a} is halogen. In some embodiments, at least one instance of R^{3a} is —F, —Cl, —Br, or —I. In some embodiments, at least one instance of R^{3a} is —F or —Cl. In some embodiments, at least one instance of R^{3a} is —F. In some embodiments, at least one instance of R^{3a} is —Cl. In some embodiments, at least one instance of R^{3a} is optionally substituted alkyl. In some embodiments, at least one instance of R^{3a} is optionally substituted C₁₋₃ alkyl. In some embodiments, at least one instance of R^{3a} is optionally substituted alkynyl. In some embodiments, at least one instance of R^{3a} is optionally substituted C₂₆ alkynyl. In some embodiments, at least one instance of R^{3a} is optionally substituted C₂₋₃ alkynyl. In some embodiments, at least one instance of R^{3a} is —CN. In some embodiments, at least one instance of R^{3a} is —NO₂. In some embodiments, at least one instance of R^{3a} is —OR^{1a}. In some embodiments, at least one instance of R^{3a} is —O (optionally substituted alkyl). In some embodiments, at least one instance of R^{3a} is —O (optionally substituted C₁₋₆ alkyl). In some embodiments, at least one instance of R^{3a} is —O(substituted C₁₋₆ alkyl). In some embodiments, at least one instance of R^{3a} is —O (unsubstituted C₁₋₆ alkyl). In some embodiments, at least one instance of R^{3a} is —O (optionally substituted C₁₋₃ alkyl). In some embodiments, at least one instance of R^{3a} is —OCH₃. In some embodiments, at least one instance of R^{3a} is —O (optionally substituted C₁₋₆ alkyl) and halogen. In some embodiments, at least one instance of R^{3a} is —OCH₃, and at least one instance of R^{3a} is —F or —Cl. In some embodiments, at least one instance of R^{3a} is —N(R^{1a})₂. In some embodiments, at least one instance of R^{3a} is —NR^{1a}C(═O)R^{1a}. In some embodiments, at least one instance of R^{3a} is —C(═O)R^{1a}. In some embodiments, at least one instance of R^{3a} is optionally substituted carbocyclyl. In some embodiments, at least one instance of R^{3a} is optionally substituted C₃₋₄ carbocyclyl. In some embodiments, at least one instance of R^{3a} is optionally substituted heterocyclyl. In some embodiments, at least one instance of R^{3a} is optionally substituted C₃₋₄ heterocyclyl.

In some embodiments, at least one instance of R^{3a} is optionally substituted phenyl. In some embodiments, at least one instance of R^{3a} is substituted phenyl. In some embodiments, at least one instance of R^{3a} is unsubstituted phenyl.

In some embodiments, at least one instance of R^{3a} is optionally substituted heteroaryl. In some embodiments, at least one instance of R^{3a} is optionally substituted 5-6 membered heteroaryl containing 1, 2, 3, or 4 ring heteroatoms selected from N, O, and S. In some embodiments, at least one instance of R^{3a} is optionally substituted 5-6 membered heteroaryl containing 1 ring S atom; 2 ring N atoms; 1 ring N atom and 1 ring O atom; 1 ring N atom and 1 ring S atom; 2 ring N atoms and 1 ring S atom; 3 ring N atoms; or 4 ring N atoms. In some embodiments, at least one instance of R^{3a} is optionally substituted 8-10 membered heteroaryl containing 1, 2, 3, or 4 ring heteroatoms selected from N, O, and S. In some embodiments, at least one instance of R^{3a} is optionally substituted 8-10 membered heteroaryl containing 2 ring N atoms; 1 ring N atom and 1 ring S atom; 2 ring N atoms and 1 ring S atom; 2 ring N atoms and 1 ring O atom; or 3 ring N atoms and 1 ring S atom.

In some embodiments, at least one instance of R^{3a} is optionally substituted triazolyl. In some embodiments, at least one instance of R^{3a} is substituted triazolyl. In some embodiments, at least one instance of R^{3a} is triazolyl, wherein the triazolyl is substituted with halogen, optionally substituted alkyl, optionally substituted heterocyclyl, optionally substituted aryl, and/or optionally substituted heteroaryl. In some embodiments, at least one instance of R^{3a} is triazolyl, wherein the triazolyl is substituted with —Cl, optionally substituted C₁₋₆ alkyl, optionally substituted 4-6 membered heterocyclyl, optionally substituted phenyl, or optionally substituted 5-6 membered heteroaryl. In some embodiments, at least one instance of R^{3a} is triazolyl, wherein the triazolyl is substituted with-Cl. In some embodiments, at least one instance of R^{3a} is triazolyl, wherein the triazolyl is substituted with substituted C₁₋₆ alkyl. In some embodiments, at least one instance of R^{3a} is triazolyl, wherein the triazolyl is substituted with substituted methyl. In some embodiments, at least one instance of R^{3a} is unsubstituted triazolyl. In some embodiments, at least one instance of R^{3a} is optionally substituted tetrazolyl.

In some embodiments, m is 0, 1, 2, 3, 4, or 5. In some embodiments, m is 0. In some embodiments, m is 1. In some embodiments, m is 2. In some embodiments, m is 3. In some embodiments, m is 4. In some embodiments, m is 5. In some embodiments, m is 0, 1, or 2. In some embodiments, m is 0 or 1.

In some embodiments, R³ is

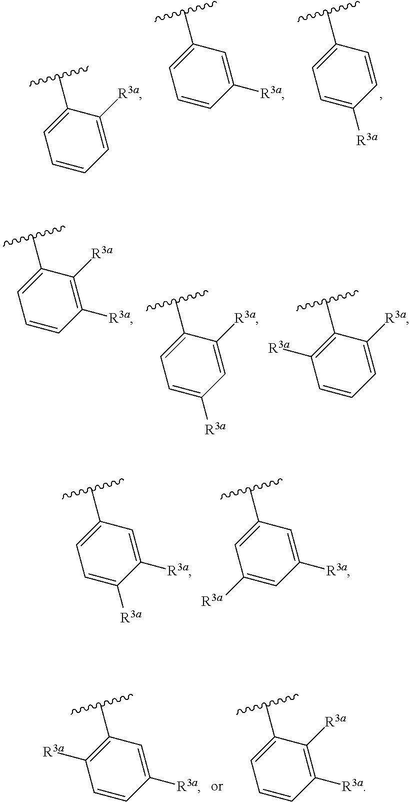

In some embodiments, R³ is

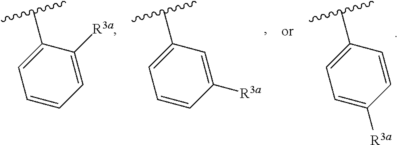

In some embodiments, R³ is

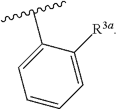

In some embodiments, R³ is

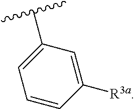

In some embodiments, R³ is

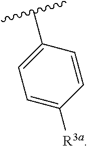

In some embodiments R³ is

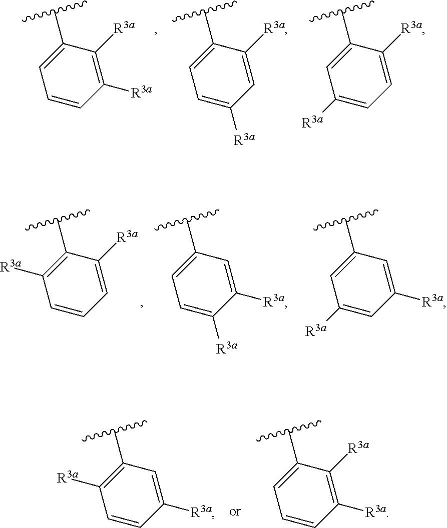

In some embodiments, R³ is

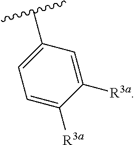

In some embodiments, R³ is

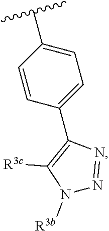

wherein R^{3b} is hydrogen, optionally substituted alkyl, optionally substituted heterocyclyl, optionally substituted aryl, or optionally substituted heteroaryl; and R^{3c} is hydrogen or halogen. In some embodiments, R^{3b} is hydrogen, methyl,

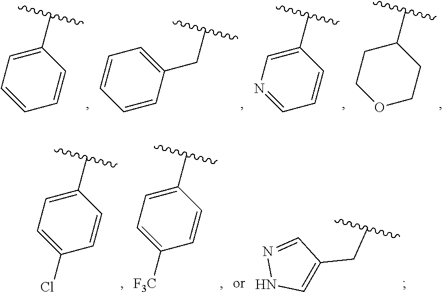

and R^{3c} is hydrogen or —Cl. In some embodiments, R^{3b} is

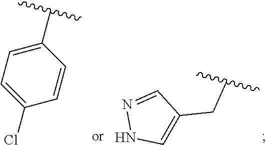 [SMILES: *c1ccc(Cl)cc1 ; *Cc1cn[nH]c1]

and R^{3c} is hydrogen.

In some embodiments, R³ is

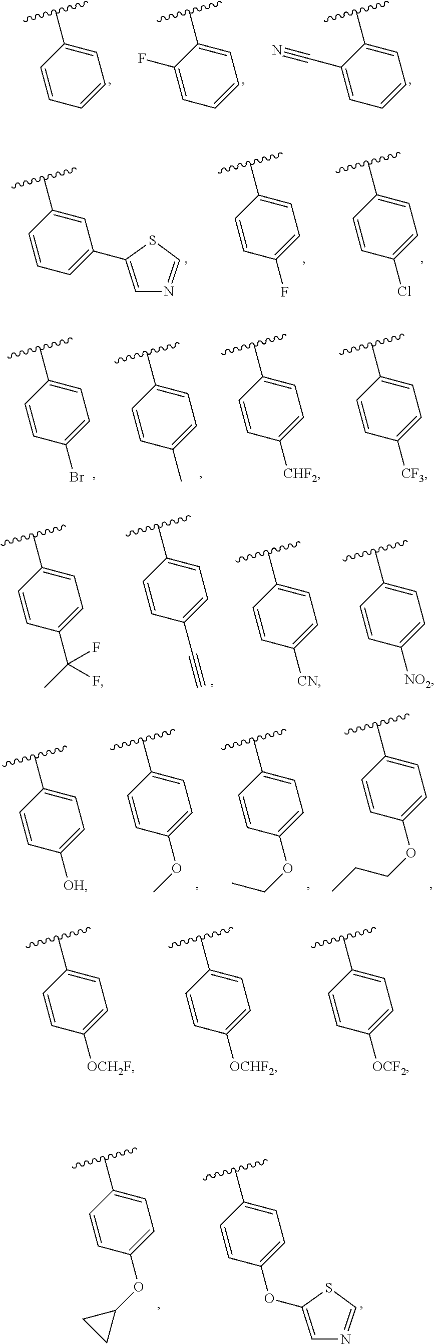

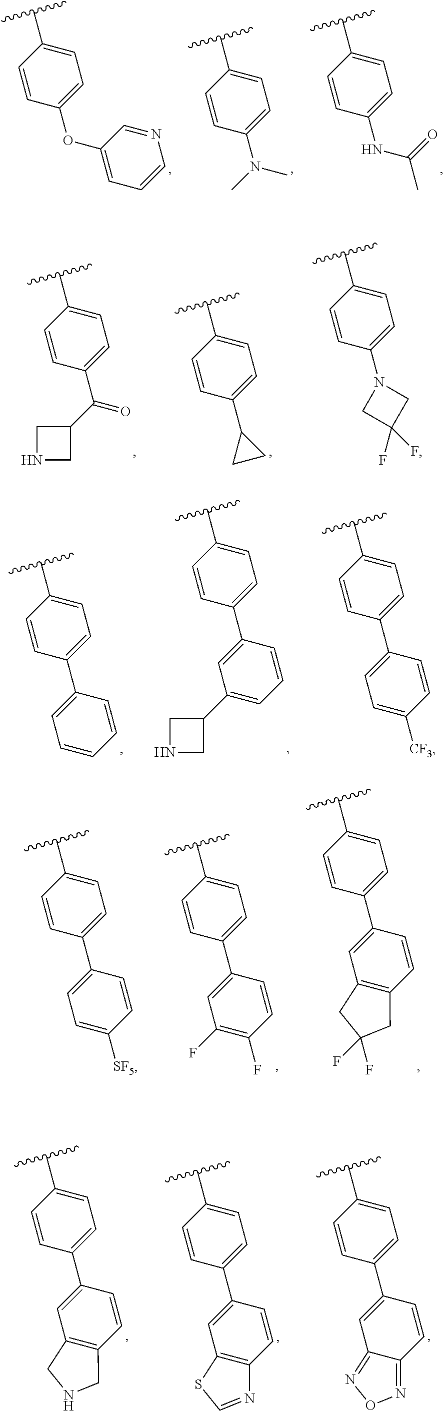

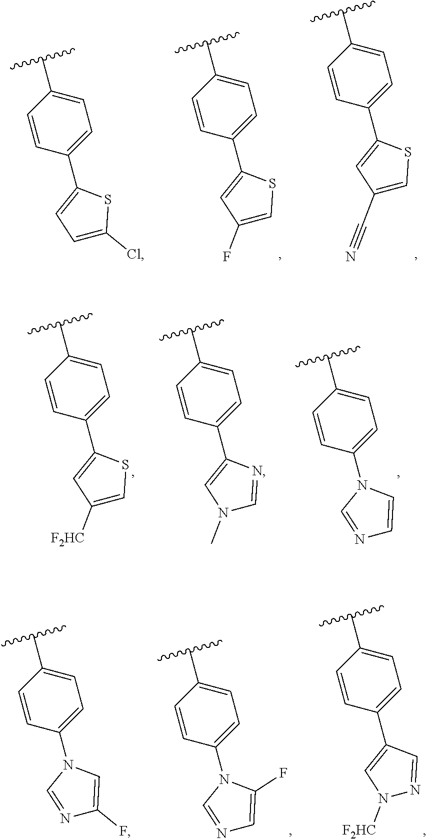

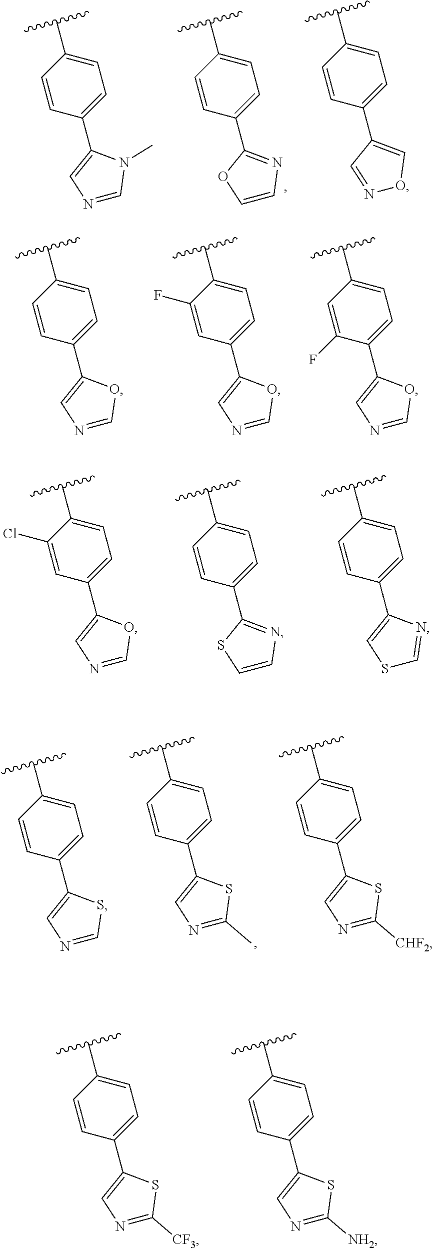

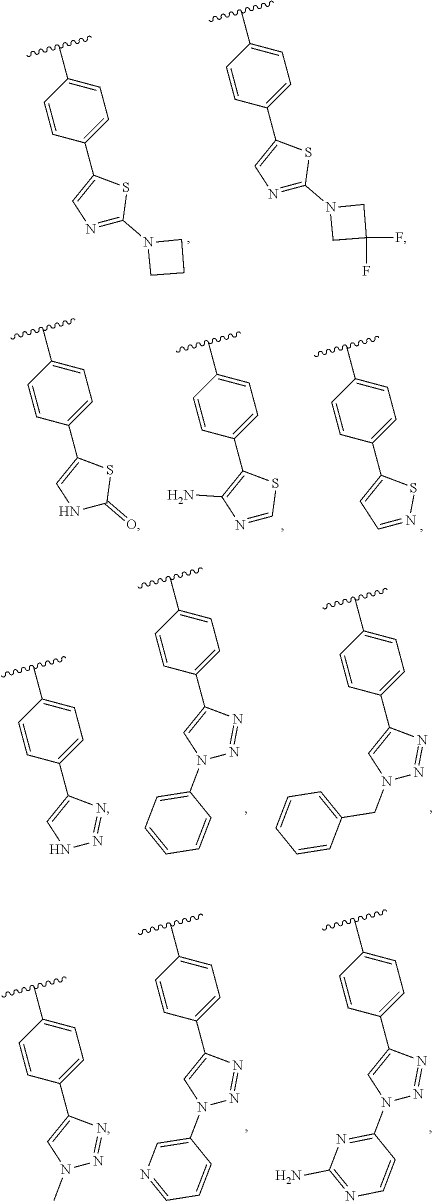

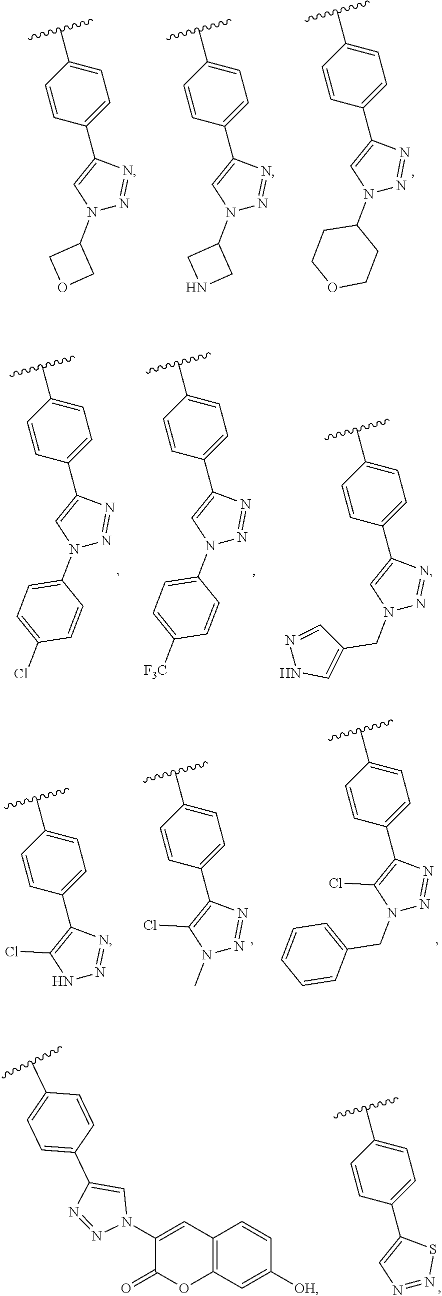

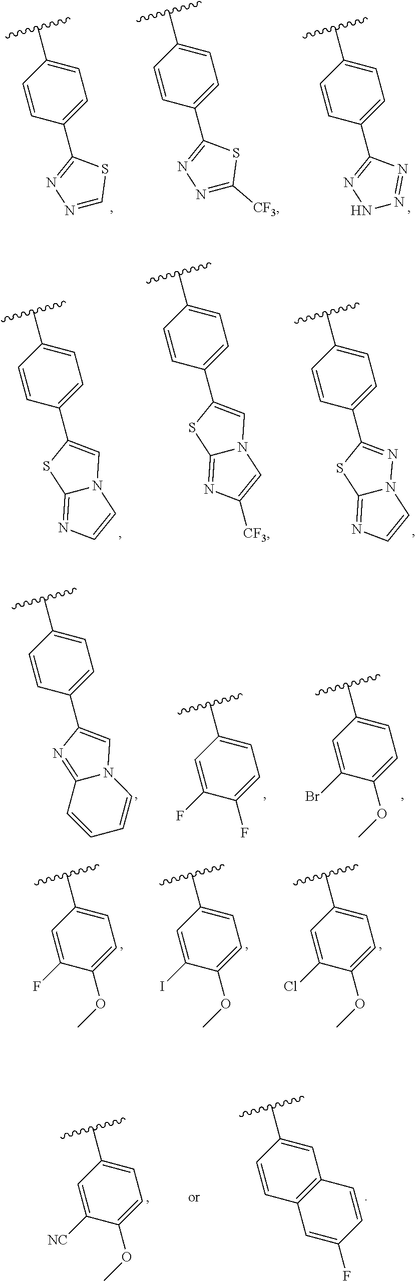

In some embodiments, R³ is

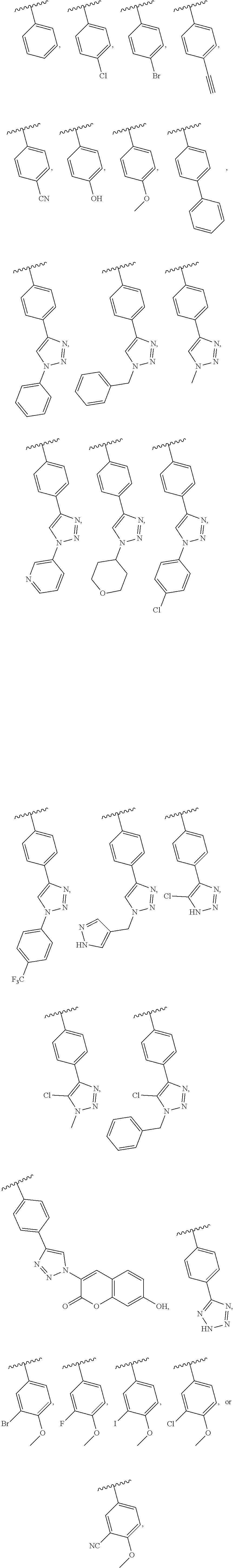

In some embodiments, R³ is

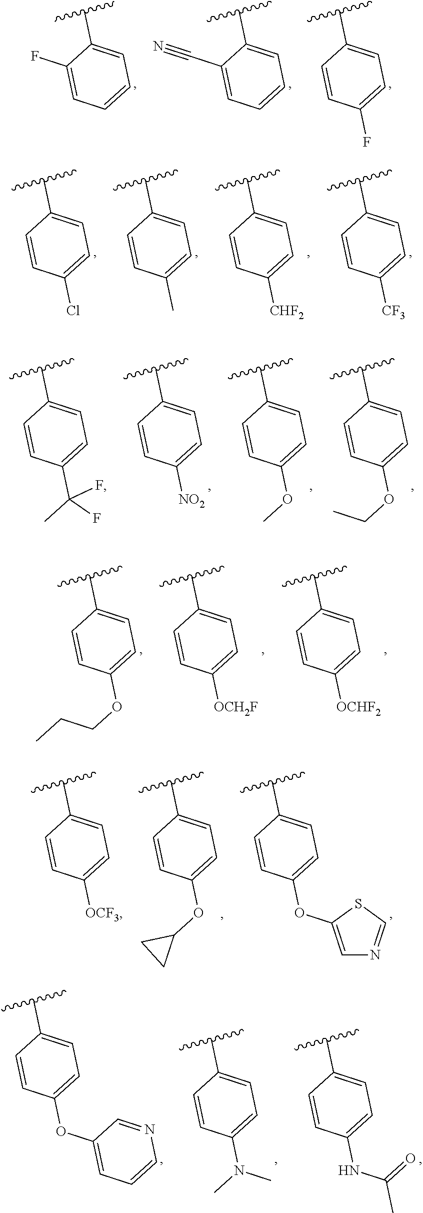

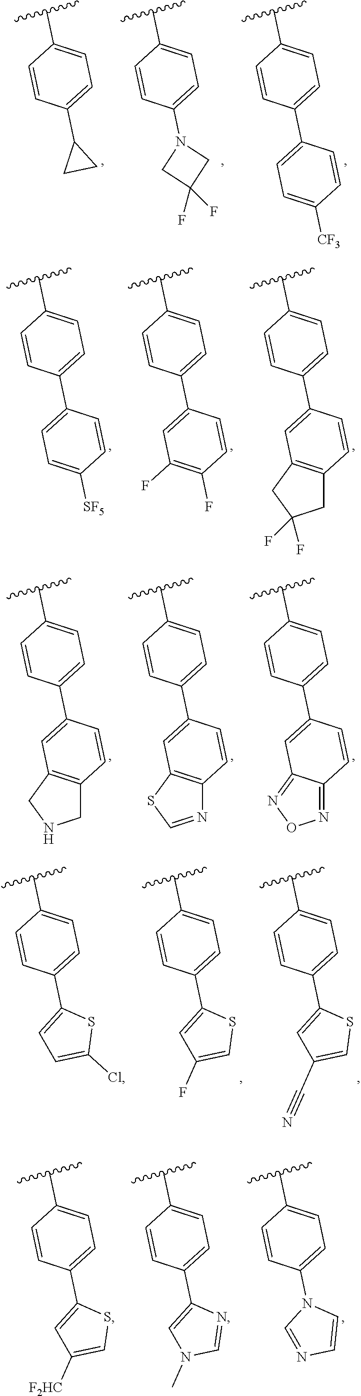

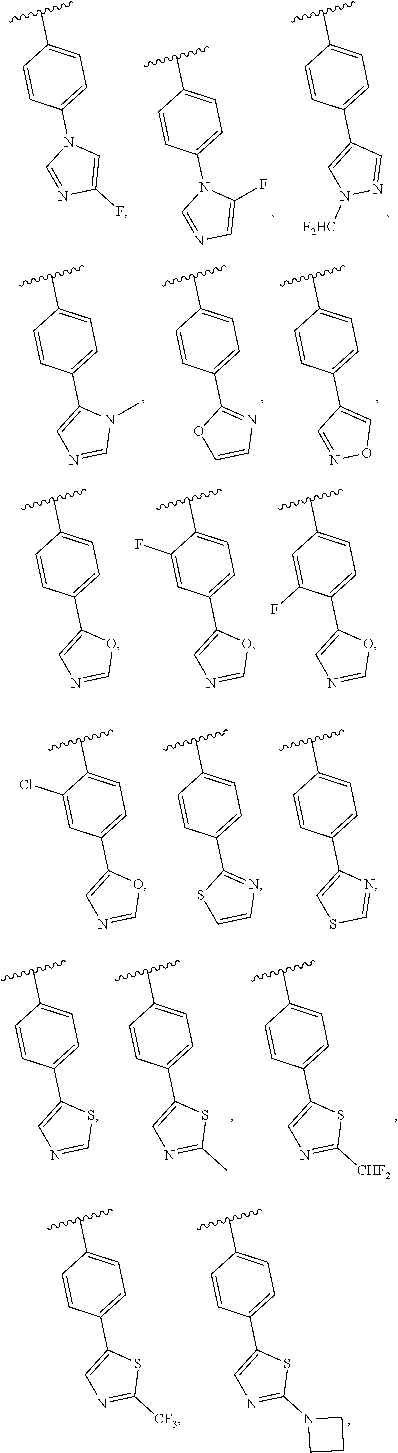

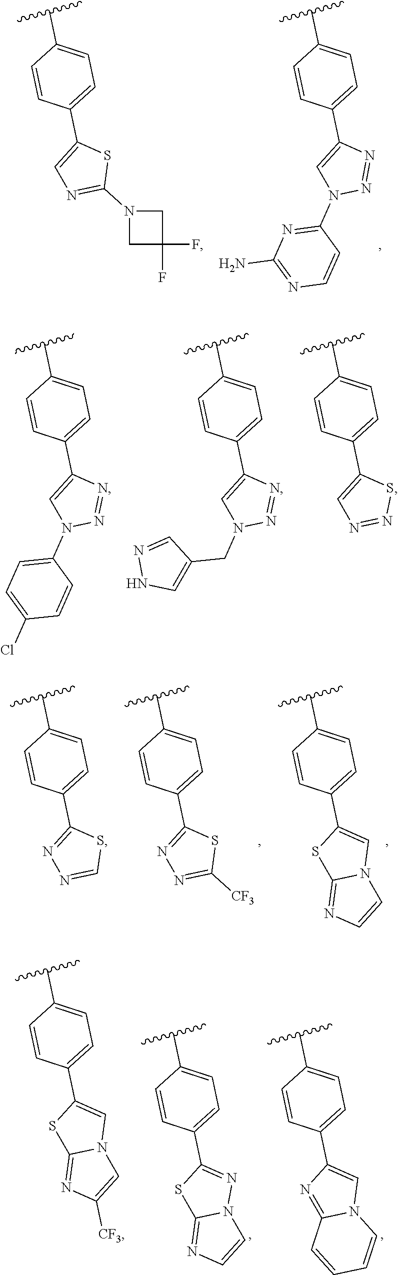

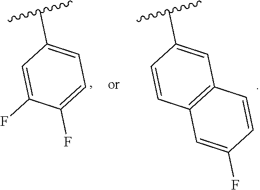 [SMILES: *c1ccc(F)c(F)c1 ; *c1ccc2cc(F)ccc2c1]

In some embodiments, R³ is

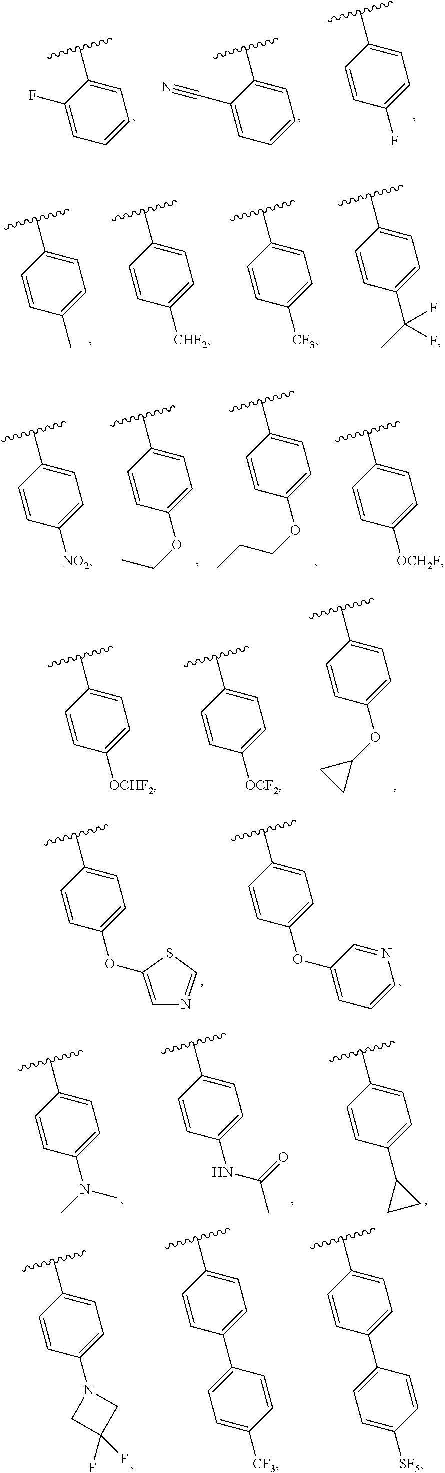

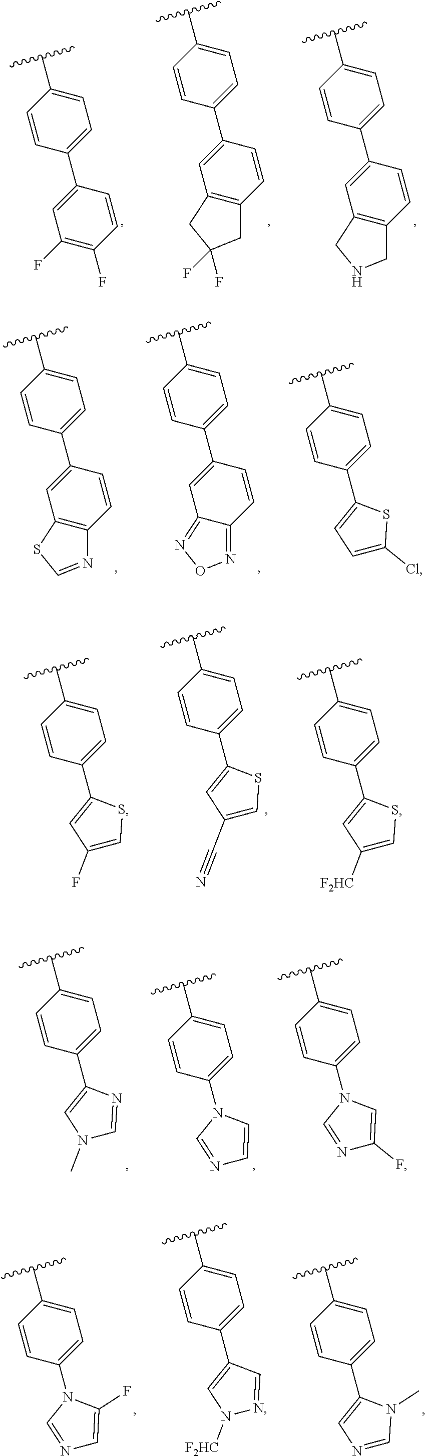

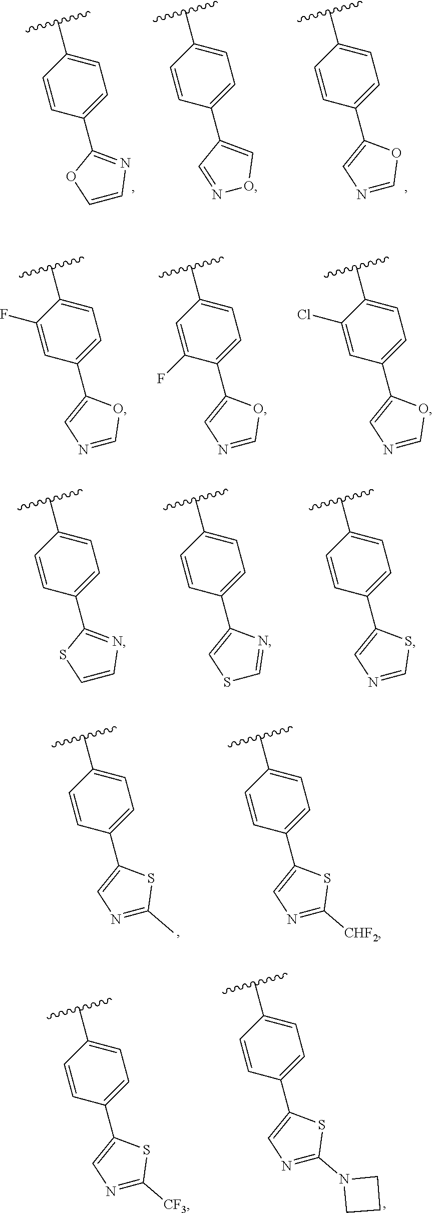

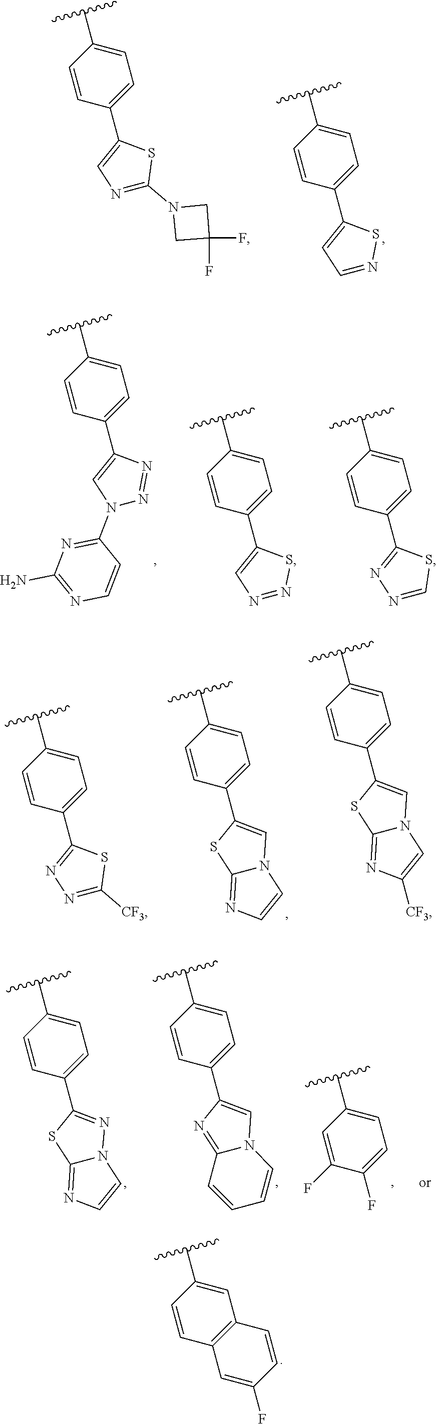

In some embodiments, R³ is

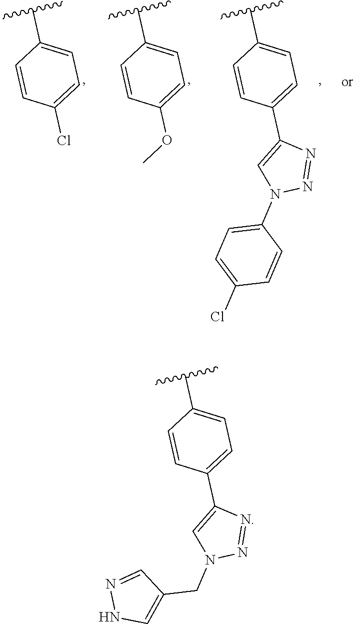

In some embodiments, R³ is

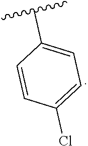 [SMILES: *c1ccc(Cl)cc1]

In some embodiments, R³ is

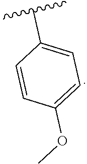 [SMILES: *c1ccc(OC)cc1]

In some embodiments, R³ is

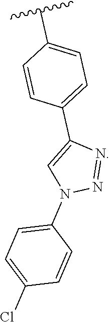 [SMILES: *c1ccc(-c2cn(-c3ccc(Cl)cc3)nn2)cc1]

In some embodiments, R³ is

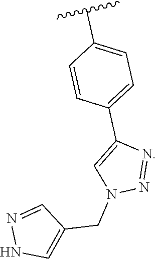 [SMILES: *c1ccc(-c2cn(Cc3cn[nH]c3)nn2)cc1]

R⁴, R^{5a}, and R^{5b}

As generally described herein, R⁴ is hydrogen or optionally substituted alkyl. In some embodiments, R⁴ is hydrogen. In some embodiments, R⁴ is optionally substituted alkyl. In some embodiments, R⁴ is optionally substituted C₁₋₁₂ alkyl. In some embodiments, R⁴ is hydrogen or optionally substituted C₁₋₆ alkyl. In some embodiments, R⁴ is optionally substituted C₁₋₆ alkyl. In some embodiments, R⁴ is optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In some embodiments, R⁴ is substituted C₁₋₆ alkyl. In some embodiments, R⁴ is substituted methyl, substituted ethyl, substituted n-propyl, substituted isopropyl, substituted n-butyl, substituted tert-butyl, substituted sec-butyl, substituted isobutyl, substituted n-pentyl, substituted 3-pentanyl, substituted amyl, substituted neopentyl, substituted 3-methyl-2-butanyl, substituted tert-amyl, or substituted n-hexyl. In some embodiments, R⁴ is hydrogen or unsubstituted C₁₋₆ alkyl. In some embodiments, R⁴ is unsubstituted C₁₋₆ alkyl. In some embodiments, R⁴ is methyl, ethyl, n-propyl, isopropyl, n-butyl, tert-butyl, sec-butyl, isobutyl, n-pentyl, 3-pentanyl, amyl, neopentyl, 3-methyl-2-butanyl, tert-amyl, or n-hexyl. In some embodiments, R⁴ is hydrogen or optionally substituted methyl. In some embodiments, R⁴ is optionally substituted methyl. In some embodiments, R⁴ is substituted methyl. In some embodiments, R⁴ is methyl. In some embodiments, R⁴ is hydrogen or methyl.

As generally described herein, R^{5a} and R^{5b} are each hydrogen, or R^{5a} and R^{5b} are joined together with their intervening atom to form optionally substituted carbocyclyl. In some embodiments, R^{5a} and R^{5b} are each hydrogen. In some embodiments, R^{5a} and R^{5b} are joined together with their intervening atom to form optionally substituted carbocyclyl. In some embodiments, R^{5a} and R^{5b} are joined together with their intervening atom to form optionally substituted C₃₋₁₀ carbocyclyl. In some embodiments, R^{5a} and R^{5b} are joined together with their intervening atom to form optionally substituted C₃₋₆ carbocyclyl. In some embodiments, R^{5a} and R^{5b} are joined together with their intervening atom to form optionally substituted cyclopropyl, optionally substituted cyclobutyl, optionally substituted cyclopentyl, or optionally substituted cyclohexyl. In some embodiments, R^{5a} and R^{5b} are joined together with their intervening atom to form substituted cyclopropyl, substituted cyclobutyl, substituted cyclopentyl, or substituted cyclohexyl. In some embodiments, R^{5a} and R^{5b} are joined together with their intervening atom to form substituted cyclopropyl. In some embodiments, R^{5a} and R^{5b} are joined together with their intervening atom to form unsubstituted cyclopropyl, unsubstituted cyclobutyl, unsubstituted cyclopentyl, or unsubstituted cyclohexyl. In some embodiments, R^{5a} and R^{5b} are joined together with their intervening atom to form unsubstituted cyclopropyl. In some embodiments, R^{5a} and R^{5b} are each hydrogen, or R^{5a} and R^{5b} are joined together with their intervening atom to form optionally substituted cyclopropyl.

### R¹¹

As generally described herein, R¹¹ is optionally substituted aryl or optionally substituted heteroaryl. In some embodiments, R¹¹ is optionally substituted aryl. In some embodiments, R¹¹ is optionally substituted C₆₋₁₄ aryl. In some embodiments, R¹¹ is optionally substituted phenyl. In some embodiments, R¹¹ is unsubstituted phenyl. In some embodiments, R¹¹ is substituted phenyl. In some embodiments, R¹¹ is phenyl substituted with halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂.

In some embodiments, R¹¹ is of formula:

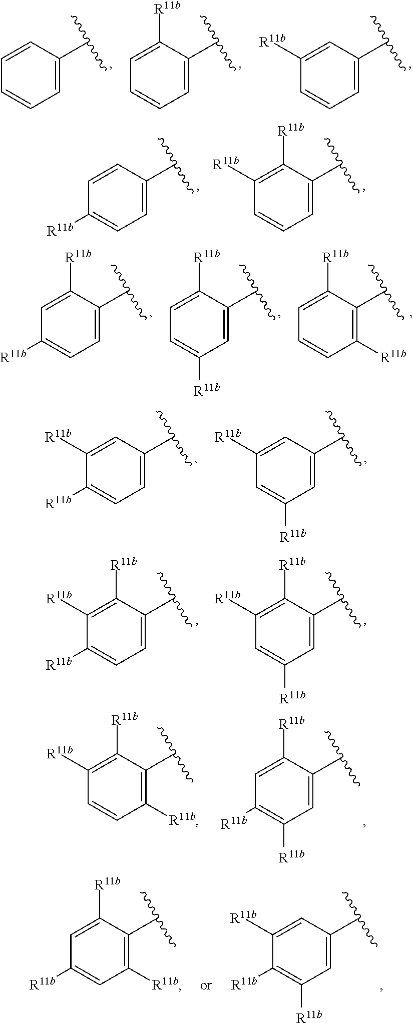

wherein each instance of R^{11b} is halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In some embodiments, R¹¹ is of formula:

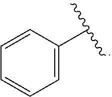 [SMILES: *c1ccccc1]

In some embodiments, R¹¹ is optionally substituted heteroaryl. In some embodiments, R¹¹ is optionally substituted 4-10 membered heteroaryl. In some embodiments, R¹¹ is optionally substituted 5-6 membered heteroaryl. In some embodiments, R¹¹ is optionally substituted monocyclic 5-6 membered heteroaryl. In some embodiments, R¹¹ is optionally substituted pyrazolyl, optionally substituted imidazolyl, optionally substituted triazolyl, optionally substituted benzimidazolyl, optionally substituted isoxazolyl, optionally substituted oxadiazolyl, optionally substituted thiazolyl, optionally substituted benzothiazolyl, or optionally substituted thiadiazolyl. In some embodiments, R¹¹ is substituted pyrazolyl, substituted imidazolyl, substituted triazolyl, substituted benzimidazolyl, substituted isoxazolyl, substituted oxadiazolyl, substituted thiazolyl, substituted benzothiazolyl, or substituted thiadiazolyl. In some embodiments, R¹¹ is unsubstituted pyrazolyl, unsubstituted imidazolyl, unsubstituted triazolyl, unsubstituted benzimidazolyl, unsubstituted isoxazolyl, unsubstituted oxadiazolyl, unsubstituted thiazolyl, unsubstituted benzothiazolyl, or unsubstituted thiadiazolyl.

In some embodiments, R¹¹ is optionally substituted pyrazolyl. In some embodiments, R¹¹ optionally substituted imidazolyl. In some embodiments, R¹¹ optionally substituted triazolyl. In some embodiments, R¹¹ optionally substituted benzimidazolyl. In some embodiments, R¹¹ optionally substituted isoxazolyl. In some embodiments, R¹¹ optionally substituted oxadiazolyl. In some embodiments, R¹¹ optionally substituted thiazolyl. In some embodiments, R¹¹ optionally substituted benzothiazolyl. In some embodiments, R¹¹ or optionally substituted thiadiazolyl. In some embodiments, R¹¹ is substituted pyrazolyl. In some embodiments, R¹¹ substituted imidazolyl. In some embodiments, R¹¹ substituted triazolyl. In some embodiments, R¹¹ substituted benzimidazolyl. In some embodiments, R¹¹ substituted isoxazolyl. In some embodiments, R¹¹ substituted oxadiazolyl. In some embodiments, R¹¹ substituted thiazolyl. In some embodiments, R¹¹ substituted benzothiazolyl. In some embodiments, R¹¹ or substituted thiadiazolyl. In some embodiments, R¹¹ is unsubstituted pyrazolyl. In some embodiments, R¹¹ unsubstituted imidazolyl. In some embodiments, R¹¹ unsubstituted triazolyl. In some embodiments, R¹¹ unsubstituted benzimidazolyl. In some embodiments, R¹¹ unsubstituted isoxazolyl. In some embodiments, R¹¹ unsubstituted oxadiazolyl. In some embodiments, R¹¹ unsubstituted thiazolyl. In some embodiments, R¹¹ unsubstituted benzothiazolyl. In some embodiments, R¹¹ or unsubstituted thiadiazolyl.

In some embodiments, R¹¹ is of formula:

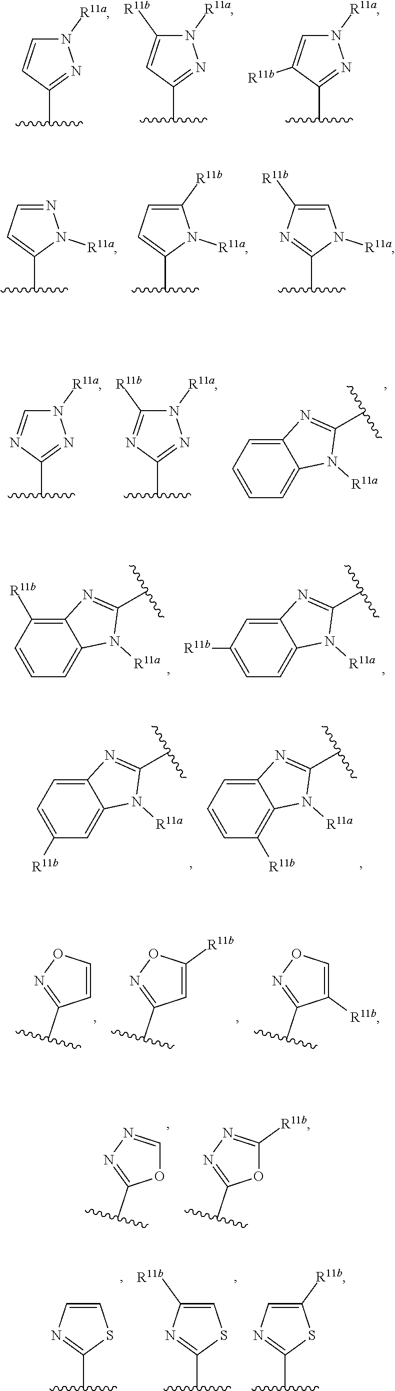

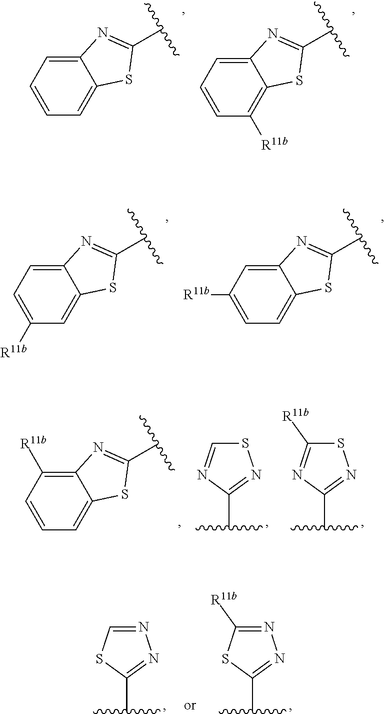

wherein:
each instance of R^{11a} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group; and
each instance of R^{11b} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In some embodiments, R¹¹ is

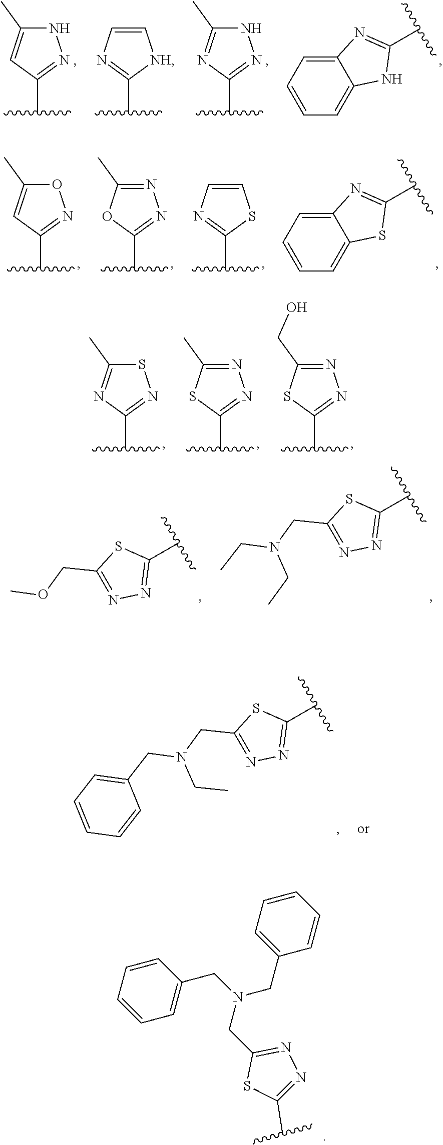

In some embodiments, R¹¹ is optionally substituted phenyl, optionally substituted pyrazolyl, optionally substituted imidazolyl, optionally substituted triazolyl, optionally substituted benzimidazolyl, optionally substituted isoxazolyl, optionally substituted oxadiazolyl, optionally substituted thiazolyl, optionally substituted benzothiazolyl, or optionally substituted thiadiazolyl. In some embodiments, R¹¹ is

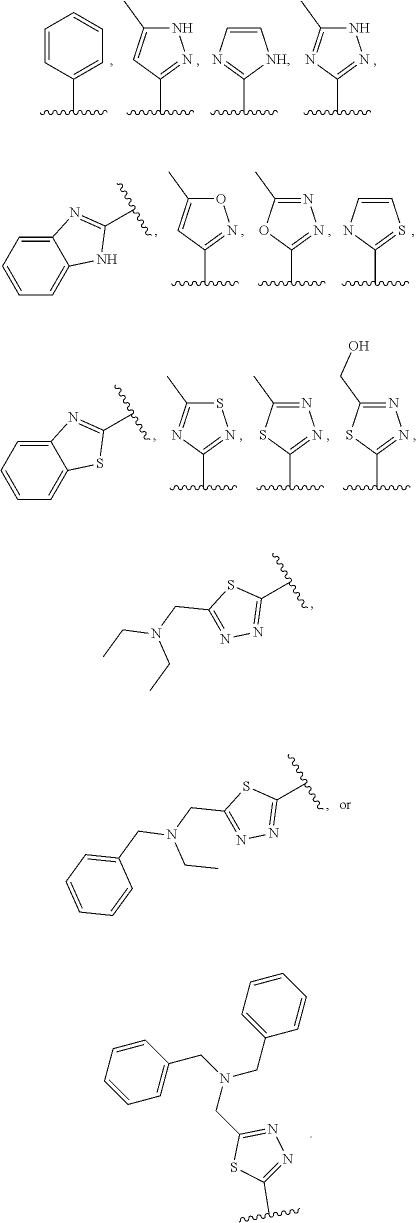

In some embodiments, R¹¹ is

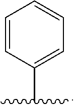 [SMILES: *c1ccccc1]

In some embodiments, R¹¹ is

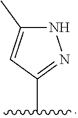 [SMILES: *c1cc(C)[nH]n1]

In some embodiments, R¹¹ is

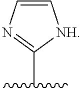 [SMILES: *c1ncc[nH]1]

In some embodiments, R¹¹ is

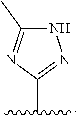 [SMILES: *c1n[nH]c(C)n1]

In some embodiments, R¹¹ is

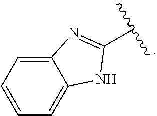 [SMILES: *c1nc2ccccc2[nH]1]

In some embodiments, R¹¹ is

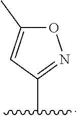 [SMILES: *c1cc(C)on1]

In some embodiments, R¹¹ is

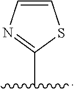 [SMILES: *c1nccs1]

In some embodiments, R¹¹ is

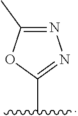 [SMILES: *c1nnc(C)o1]

In some embodiments, R¹¹ is

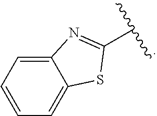 [SMILES: *c1nc2ccccc2s1]

In some embodiments, R¹¹ is

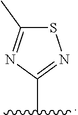 [SMILES: *c1nsc(C)n1]

In some embodiments, R¹¹ is

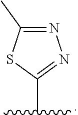 [SMILES: *c1nnc(C)s1]

In some embodiments, R¹¹ is

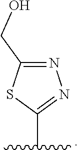 [SMILES: *c1nnc(CO)s1]

In some embodiments, R¹¹ is

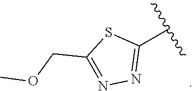 [SMILES: *c1nnc(COC)s1]

In some embodiments, R¹¹ is

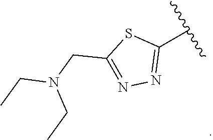 [SMILES: *c1nnc(CN(CC)CC)s1]

In some embodiments, R¹¹ is

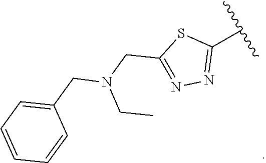 [SMILES: *c1nnc(CN(CC)Cc2ccccc2)s1]

In some embodiments, R¹¹ is

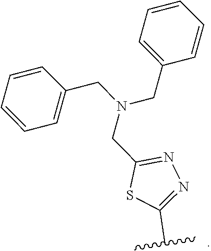 [SMILES: *c1nnc(CN(Cc2ccccc2)Cc2ccccc2)s1]

### R^{A}

As generally described herein, each instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring.

In some embodiments, at least one instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring. In some embodiments, at least one instance of R^{A} is hydrogen. In some embodiments, at least one instance of R^{A} is optionally substituted acyl. In some embodiments, at least one instance of R^{A} is optionally substituted C₁₋₁₂ alkyl. In some embodiments, at least one instance of R^{A} is optionally substituted C₁₋₆ alkyl. In some embodiments, at least one instance of R^{A} is unsubstituted C₁₋₆ alkyl. In some embodiments, at least one instance of R^{A} is substituted C₁₋₆ alkyl. In some embodiments, at least one instance of R^{A} is optionally substituted methyl, optionally substituted ethyl, optionally substituted n-propyl, optionally substituted isopropyl, optionally substituted n-butyl, optionally substituted tert-butyl, optionally substituted sec-butyl, optionally substituted isobutyl, optionally substituted n-pentyl, optionally substituted 3-pentanyl, optionally substituted amyl, optionally substituted neopentyl, optionally substituted 3-methyl-2-butanyl, optionally substituted tert-amyl, or optionally substituted n-hexyl. In some embodiments, at least one instance of R^{A} is optionally substituted C₂₋₁₂ alkenyl. In some embodiments, at least one instance of R^{A} is optionally substituted C₂₋₆ alkenyl. In some embodiments, at least one instance of R^{A} is optionally substituted ethenyl, optionally substituted 1-propenyl, optionally substituted 2-propenyl, optionally substituted 1-butenyl, optionally substituted 2-butenyl, optionally substituted butadienyl, optionally substituted pentenyl, optionally substituted pentadienyl, or optionally substituted hexenyl. In some embodiments, at least one instance of R^{A} is optionally substituted C₂₋₁₂ alkynyl. In some embodiments, at least one instance of R^{A} is optionally substituted C₂₋₆ alkynyl. In some embodiments, at least one instance of R^{A} is optionally substituted ethynyl, optionally substituted 1-propynyl, optionally substituted 2-propynyl, optionally substituted 1-butynyl, optionally substituted 2-butynyl, optionally substituted pentynyl, or optionally substituted hexynyl. In some embodiments, at least one instance of R^{A} is optionally substituted heteroC₁₋₁₂ alkyl. In some embodiments, at least one instance of R^{A} is optionally substituted heteroC₁₋₆ alkyl. In some embodiments, at least one instance of R^{A} is optionally substituted heteroC₁₋₁₂ alkenyl. In some embodiments, at least one instance of R^{A} is optionally substituted heteroC₁₋₆ alkenyl. In some embodiments, at least one instance of R^{A} is optionally substituted heteroC₁₋₁₂ alkynyl. In some embodiments, at least one instance of R^{A} is optionally substituted heteroC₁₋₆ alkynyl. In some embodiments, at least one instance of R^{A} is optionally substituted C₃₋₁₄ cycloalkyl. In some embodiments, at least one instance of R^{A} is optionally substituted 5-10 membered heterocyclyl. In some embodiments, at least one instance of R^{A} is optionally substituted 6-14 membered aryl. In some embodiments, at least one instance of R^{A} is optionally substituted 5-14 membered heteroaryl. In some embodiments, at least one instance of R^{A} is a nitrogen protecting group when attached to a nitrogen atom. In some embodiments, at least one instance of R^{A} is an oxygen protecting group when attached to an oxygen atom. In some embodiments, at least one instance of R^{A} is a sulfur protecting group when attached to a sulfur atom. In some embodiments, at least two instances of R^{A} are joined together with their intervening atom to form an optionally substituted 5-10 membered heterocyclic ring. In some embodiments, at least two instances of R^{A} are joined together with their intervening atom to form an optionally substituted 5-14 membered heteroaryl ring.

### Subgenera

In some embodiments, X is —CH(R²)—; R¹ is optionally substituted heterocyclyl, optionally substituted aryl, or optionally substituted heteroaryl; R² is hydrogen; and n is 0 or 1. In some embodiments, X is —CH(R²)—; R¹ is optionally substituted heterocyclyl, optionally substituted aryl, or optionally substituted heteroaryl; R² is hydrogen; and n is 0. In some embodiments, X is —CH(R²)—; R¹ is optionally substituted heterocyclyl, optionally substituted aryl, or optionally substituted heteroaryl; R² is hydrogen; and n is 1. In some embodiments, X is —CH(R²)—; R¹ is optionally substituted 4-6 membered heterocyclyl, optionally substituted phenyl, or optionally substituted 5-6 membered heteroaryl; R² is hydrogen; and n is 0 or 1. In some embodiments, X is —CH(R²)—; R¹ is optionally substituted 4-6 membered heterocyclyl, optionally substituted phenyl, or optionally substituted 5-6 membered heteroaryl; R² is hydrogen; and n is 0. In some embodiments, X is —CH(R²)—; R¹ is optionally substituted 4-6 membered heterocyclyl, optionally substituted phenyl, or optionally substituted 5-6 membered heteroaryl; R² is hydrogen; and n is 1.

In some embodiments, X is —CH(R²)—; R¹ is optionally substituted 4-6 membered heterocyclyl; R² is hydrogen; and n is 0 or 1. In some embodiments, X is —CH(R²)—; R¹ is optionally substituted 4-6 membered heterocyclyl; R² is hydrogen; and n is 0. In some embodiments, X is —CH(R²)—; R¹ is optionally substituted 4-6 membered heterocyclyl; R² is hydrogen; and n is 1. In some embodiments, X is —CH(R²)—; R¹ is optionally substituted phenyl; R² is hydrogen; and n is 0 or 1. In some embodiments, X is —CH(R²)—; R¹ is optionally substituted phenyl; R² is hydrogen; and n is 0. In some embodiments, X is —CH(R²)—; R¹ is optionally substituted phenyl; R² is hydrogen; and n is 1. In some embodiments, X is —CH(R²)—; R¹ is optionally substituted 5-6 membered heteroaryl; R² is hydrogen; and n is 0 or 1. In some embodiments, X is —CH(R²)—; R¹ is optionally substituted 5-6 membered heteroaryl; R² is hydrogen; and n is 0. In some embodiments, X is —CH(R²)—; R¹ is optionally substituted 5-6 membered heteroaryl; R² is hydrogen; and n is 1.

In some embodiments, X is —CH(R²)—; R¹ is optionally substituted tetrahydrofuranyl, optionally substituted tetrahydropyranyl, optionally substituted azetidinyl, optionally substituted pyrrolidinyl, optionally substituted imidazolidinyl, optionally substituted piperidinyl, optionally substituted piperazinyl, optionally substituted tetrahydrothiopyranyl, optionally substituted pyridyl, optionally substituted pyrazinyl, optionally substituted pyrimidinyl, optionally substituted pyridazinyl, optionally substituted pyrazolyl, optionally substituted imidazolyl, optionally substituted triazolyl, optionally substituted tetrazolyl, optionally substituted thiophenyl, optionally substituted oxazolyl, optionally substituted isoxazolyl, optionally substituted oxadiazolyl, optionally substituted thiazolyl, optionally substituted thiadiazolyl, or phenyl optionally substituted with halogen, optionally substituted alkyl, optionally substituted aryl, —OR^{1a}, —N(R^{1a})₂, —C(═NR^{A})N(R^{A})₂, and/or —B(OR^{A})₂; R² is hydrogen; and n is 0 or 1.

In some embodiments, X is —CH(R²)—; R¹ is optionally substituted tetrahydrofuranyl, optionally substituted tetrahydropyranyl, optionally substituted azetidinyl, optionally substituted pyrrolidinyl, optionally substituted imidazolidinyl, optionally substituted piperidinyl, optionally substituted piperazinyl, or optionally substituted tetrahydrothiopyranyl; R² is hydrogen; and n is 0 or 1. In some embodiments, X is —CH(R²)—; R¹ is optionally substituted tetrahydrofuranyl, optionally substituted azetidinyl, optionally substituted pyrrolidinyl, optionally substituted imidazolidinyl, optionally substituted piperidinyl, optionally substituted piperazinyl, or optionally substituted tetrahydrothiopyranyl; R² is hydrogen; and n is 0 or 1. In some embodiments, X is —CH(R²)—; R¹ is

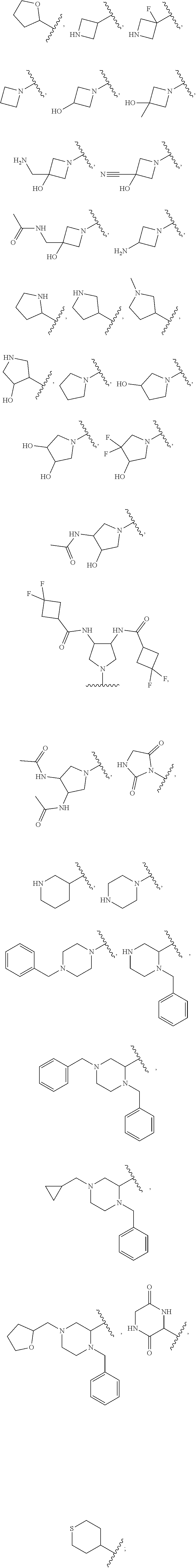

R² is hydrogen; and n is 0 or 1.

In some embodiments, X is —CH(R²)—; R¹ is optionally substituted pyridyl, optionally substituted pyrazinyl, optionally substituted pyrimidinyl, optionally substituted pyridazinyl, optionally substituted pyrazolyl, optionally substituted imidazolyl, optionally substituted triazolyl, optionally substituted tetrazolyl, optionally substituted thiophenyl, optionally substituted oxazolyl, optionally substituted isoxazolyl, optionally substituted oxadiazolyl, optionally substituted thiazolyl, or optionally substituted thiadiazolyl; R² is hydrogen; and n is 0 or 1. In some embodiments, X is —CH(R²)—; R¹ is optionally substituted pyridyl, optionally substituted pyrimidinyl, optionally substituted pyridazinyl, optionally substituted pyrazolyl, optionally substituted imidazolyl, optionally substituted triazolyl, optionally substituted tetrazolyl, optionally substituted oxazolyl, optionally substituted isoxazolyl, or optionally substituted thiazolyl; R² is hydrogen; and n is 0 or 1. In some embodiments, X is —CH(R²)—; R¹ is

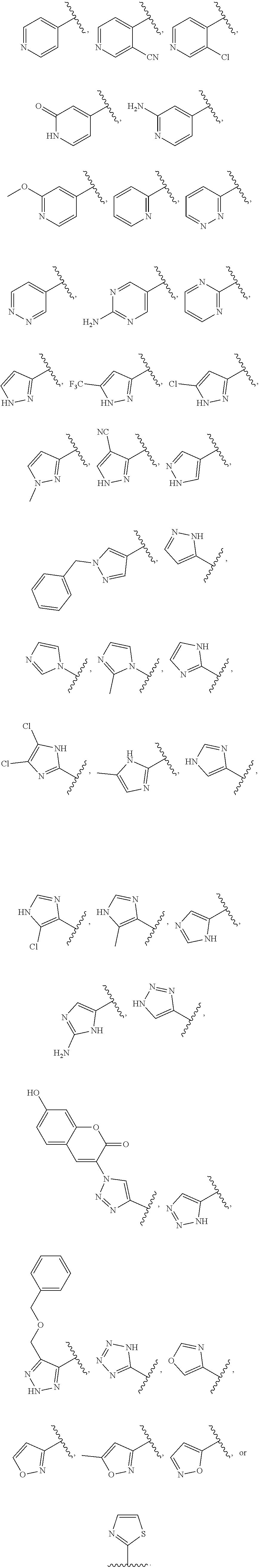

R² is hydrogen; and n is 0 or 1.

In some embodiments, X is —CH(R²)—; R¹ is phenyl optionally substituted with halogen, optionally substituted alkyl, optionally substituted aryl, —OR^{1a}, —N(R^{1a})₂, —C(═NR^{A})N(R^{A})₂, and/or —B(OR^{A})₂; R² is hydrogen; and n is 0 or 1. In some embodiments, X is —CH(R²)—; R¹ is

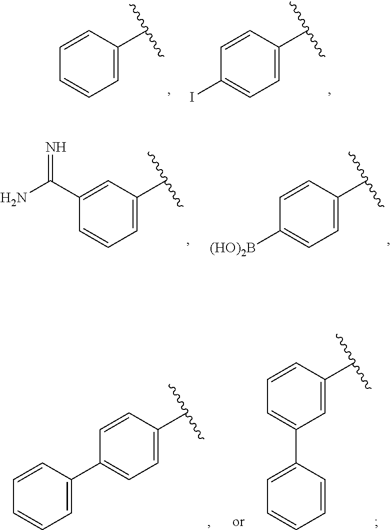 [SMILES: *c1ccccc1 ; *c1ccc(I)cc1 ; *c1cccc(C(=N)N)c1 ; *c1ccc(B(O)O)cc1 ; *c1ccc(-c2ccccc2)cc1 ; *c1cccc(-c2ccccc2)c1]

R² is hydrogen; and n is 0 or 1.

In some embodiments, the compound of Formula (I) is of Formula (I-a):

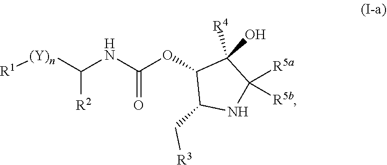

or a pharmaceutically acceptable salt thereof.

In some embodiments, the compound of Formula (I-a) is of Formula (I-a-1), (I-a-2), (I-a-3), or (I-a-4):

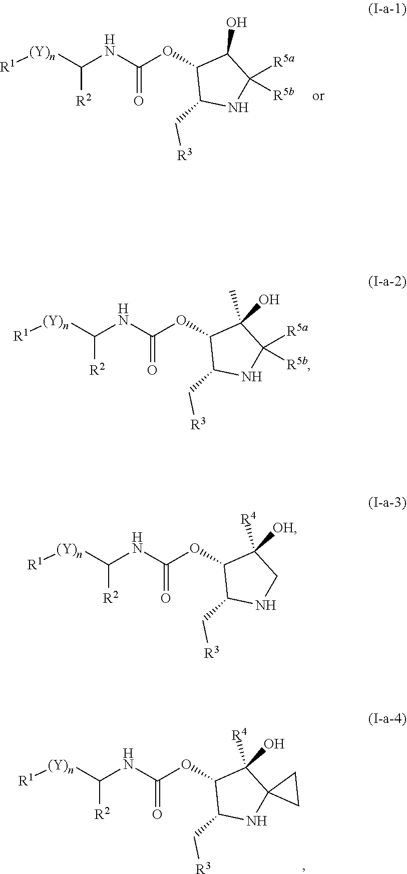

or a pharmaceutically acceptable salt thereof.

In some embodiments, the compound of Formula (I) is of Formula (I-b):

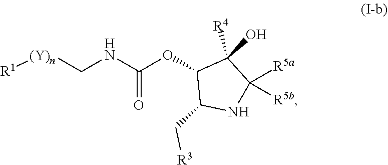

or a pharmaceutically acceptable salt thereof.

In some embodiments, the compound of Formula (I-b) is of Formula (I-b-1), (I-b-2), (I-b-3), or (I-b-4):

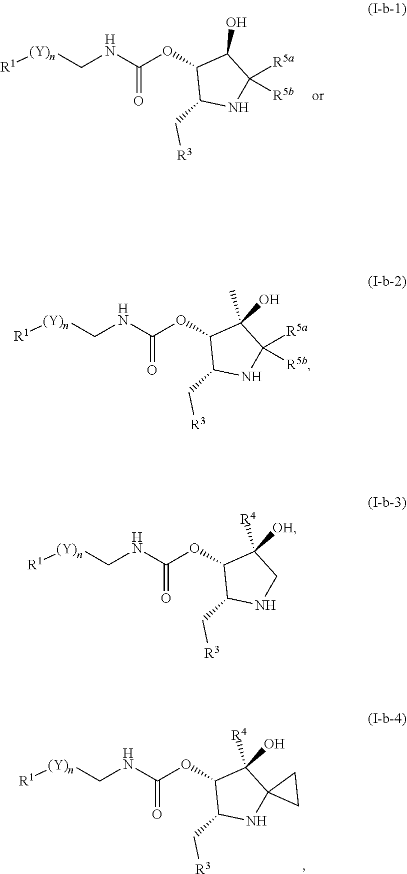

or a pharmaceutically acceptable salt thereof.

In some embodiments, the compound of Formula (I) is of Formula (I-c):

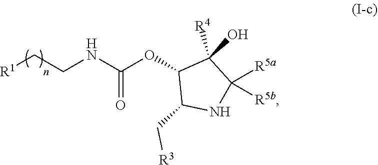

or a pharmaceutically acceptable salt thereof.

In some embodiments, the compound of Formula (I-c) is of Formula (I-c-1) or (I-c-2):

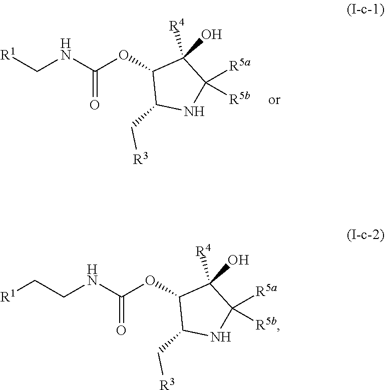

or a pharmaceutically acceptable salt thereof.

In some embodiments, the compound of Formula (I) is of Formula (II):

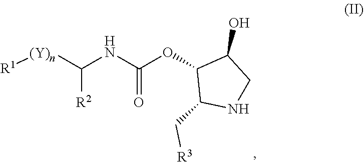

or a pharmaceutically acceptable salt thereof.

In some embodiments, the compound of Formula (II) is of Formula (II-a):

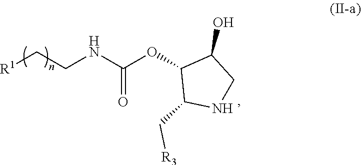

or a pharmaceutically acceptable salt thereof.

In some embodiments, the compound of Formula (II-a) is of Formula (II-a-1) or (II-a-2):

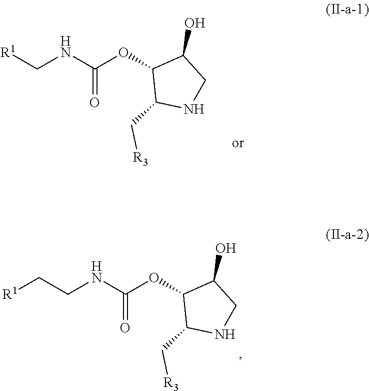

or a pharmaceutically acceptable salt thereof.

In some embodiments, the compound of Formula (I) is of Formula (III):

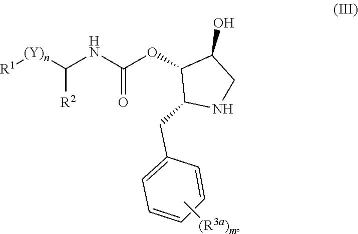

or a pharmaceutically acceptable salt thereof, wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring; and
m is 0, 1, 2, 3, 4, or 5.

In some embodiments, the compound of Formula (III) is of Formula (III-a-1), (III-a-2), or (III-a-3):

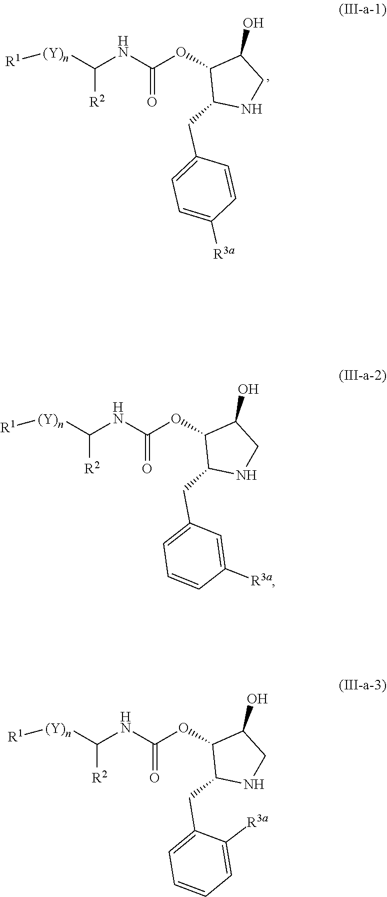

or a pharmaceutically acceptable salt thereof.

In some embodiments, the compound of Formula (III) is of Formula (III-b):

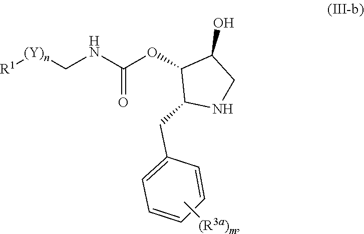

or a pharmaceutically acceptable salt thereof.

In some embodiments, the compound of Formula (III-b) is of Formula (III-b-1), (III-b-2), or (III-b-3):

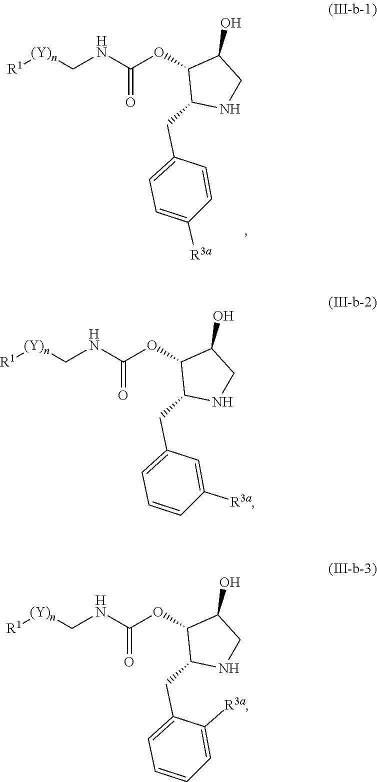

or a pharmaceutically acceptable salt thereof.

In some embodiments, the compound of Formula (III) is of Formula (III-c):

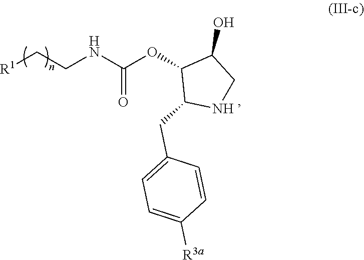

or a pharmaceutically acceptable salt thereof.

In some embodiments, the compound of Formula (III-c) is of Formula (III-c-1) or (III-c-2):

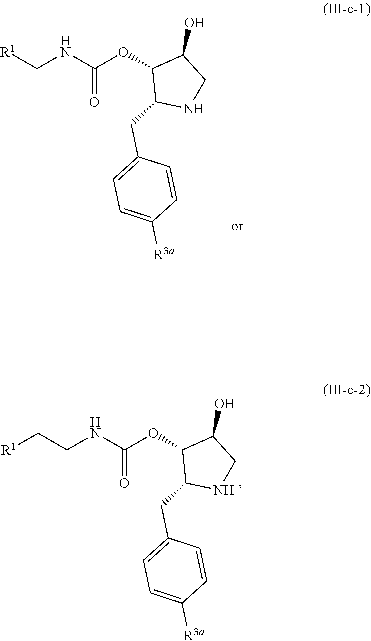

or a pharmaceutically acceptable salt thereof.

In some embodiments, the compound of Formula (I) is of Formula (IV):

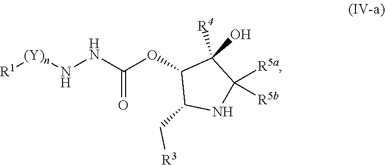

or a pharmaceutically acceptable salt thereof.

In some embodiments, the compound of Formula (IV) is of Formula (IV-a):

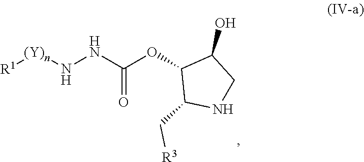

or a pharmaceutically acceptable salt thereof.

In some embodiments, the compound of Formula (IV) is of Formula (IV-b):

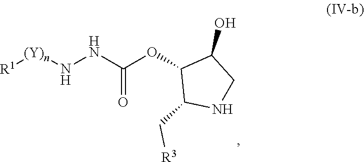

or a pharmaceutically acceptable salt thereof.

In some embodiments, the compound of Formula (IV) is of Formula (IV-c):

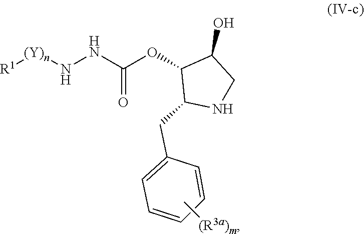

or a pharmaceutically acceptable salt thereof, wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring; and
m is 0, 1, 2, 3, 4, or 5.

In some embodiments, the compound of Formula (IV-c) is of Formula (IV-c-1), (IV-c-2), or (IV-c-3):

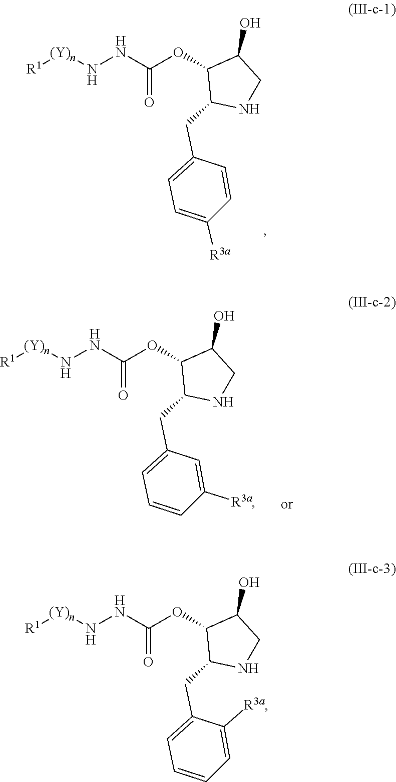

or a pharmaceutically acceptable salt thereof.

In some embodiments, the compound of Formula (V) is of Formula (V-a):

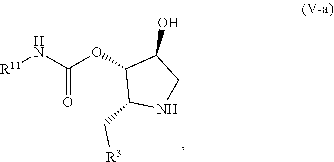

or pharmaceutically acceptable salt thereof.

In some embodiments, the compound of Formula (V) is of Formula (V-b):

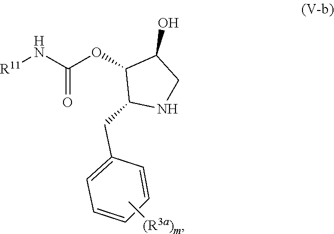

or a pharmaceutically acceptable salt thereof, wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring; and
m is 0, 1, 2, 3, 4, or 5.

In some embodiments, the compound of Formula (V-b) is of Formula (V-b-1), (V-b-2), or (V-b-3):

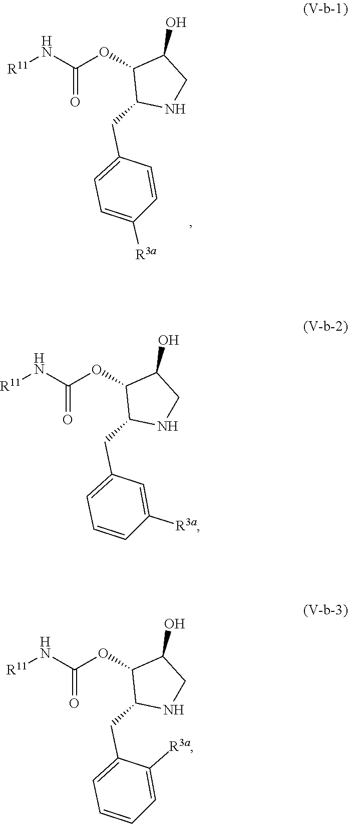

or pharmaceutically acceptable salt thereof.

In some embodiments, the compound of Formula (I), or pharmaceutically acceptable salt thereof, is selected from those in Table 1A, and pharmaceutically acceptable salts thereof: In some embodiments, the compound of Formula (I), or pharmaceutically acceptable salt thereof, is selected from those in Table 1B, and pharmaceutically acceptable salts thereof:

TABLE 1A
Compounds of Formula (I)

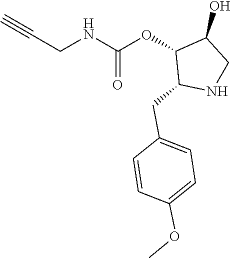 [SMILES: C#CCNC(=O)O[C@@H]1[C@@H](O)CN[C@@H]1Cc1ccc(OC)cc1]

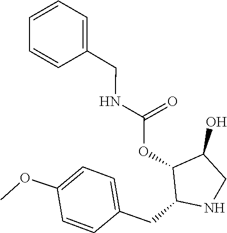 [SMILES: COc1ccc(C[C@H]2NC[C@H](O)[C@H]2OC(=O)NCc2ccccc2)cc1]

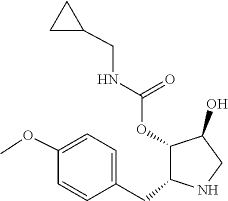 [SMILES: COc1ccc(C[C@H]2NC[C@H](O)[C@H]2OC(=O)NCC2CC2)cc1]

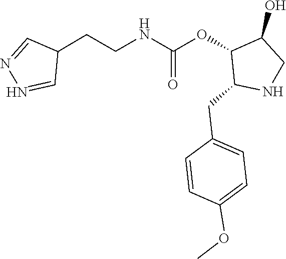 [SMILES: COc1ccc(C[C@H]2NC[C@H](O)[C@H]2OC(=O)NCCC2C=NN=C2)cc1]

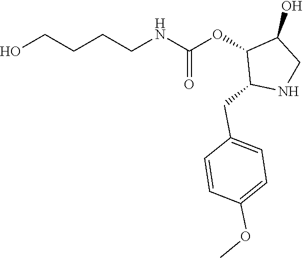 [SMILES: COc1ccc(C[C@H]2NC[C@H](O)[C@H]2OC(=O)NCCCCO)cc1]

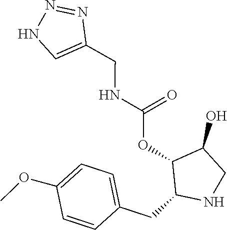 [SMILES: COc1ccc(C[C@H]2NC[C@H](O)[C@H]2OC(=O)NCc2c[nH]nn2)cc1]

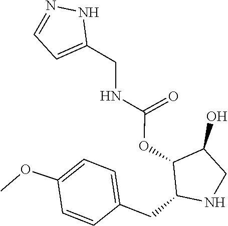 [SMILES: COc1ccc(C[C@H]2NC[C@H](O)[C@H]2OC(=O)NCc2ccn[nH]2)cc1]

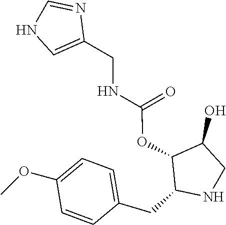 [SMILES: COc1ccc(C[C@H]2NC[C@H](O)[C@H]2OC(=O)NCc2c[nH]cn2)cc1]

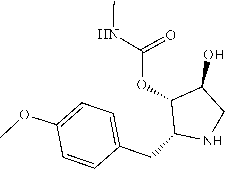 [SMILES: CNC(=O)O[C@@H]1[C@@H](O)CN[C@@H]1Cc1ccc(OC)cc1]

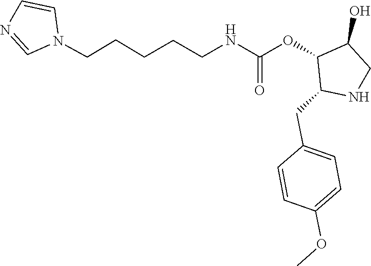 [SMILES: COc1ccc(C[C@H]2NC[C@H](O)[C@H]2OC(=O)NCCCCCn2ccnc2)cc1]

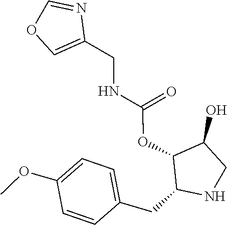 [SMILES: COc1ccc(C[C@H]2NC[C@H](O)[C@H]2OC(=O)NCc2cocn2)cc1]

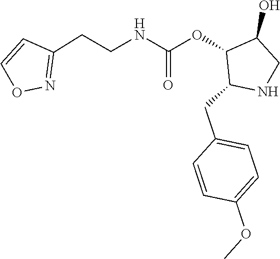 [SMILES: COc1ccc(C[C@H]2NC[C@H](O)[C@H]2OC(=O)NCCc2ccon2)cc1]

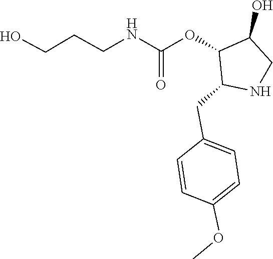 [SMILES: COc1ccc(C[C@H]2NC[C@H](O)[C@H]2OC(=O)NCCCO)cc1]

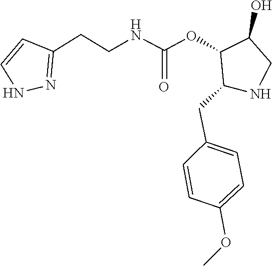 [SMILES: COc1ccc(C[C@H]2NC[C@H](O)[C@H]2OC(=O)NCCc2cc[nH]n2)cc1]

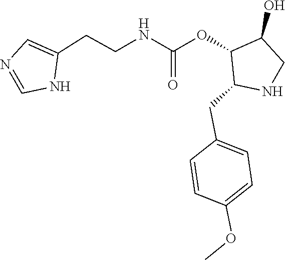 [SMILES: COc1ccc(C[C@H]2NC[C@H](O)[C@H]2OC(=O)NCCc2cnc[nH]2)cc1]

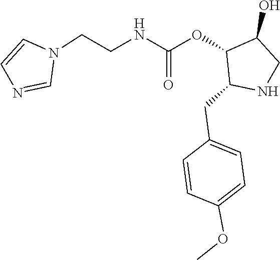 [SMILES: COc1ccc(C[C@H]2NC[C@H](O)[C@H]2OC(=O)NCCn2ccnc2)cc1]

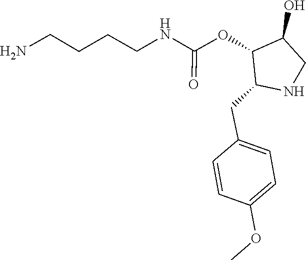 [SMILES: COc1ccc(C[C@H]2NC[C@H](O)[C@H]2OC(=O)NCCCCN)cc1]

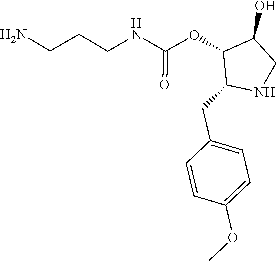 [SMILES: COc1ccc(C[C@H]2NC[C@H](O)[C@H]2OC(=O)NCCCN)cc1]

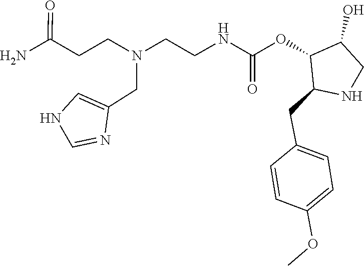 [SMILES: COc1ccc(C[C@@H]2NC[C@@H](O)[C@H]2OC(=O)NCCN(CCC(N)=O)Cc2c[nH]cn2)cc1]

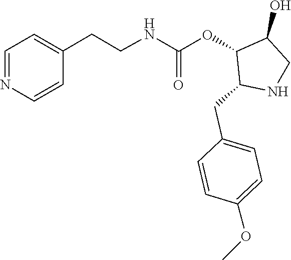 [SMILES: COc1ccc(C[C@H]2NC[C@H](O)[C@H]2OC(=O)NCCc2ccncc2)cc1]

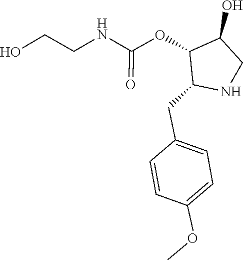 [SMILES: COc1ccc(C[C@H]2NC[C@H](O)[C@H]2OC(=O)NCCO)cc1]

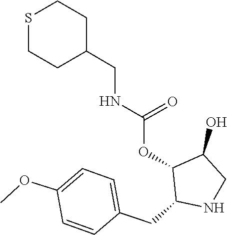 [SMILES: COc1ccc(C[C@H]2NC[C@H](O)[C@H]2OC(=O)NCC2CCSCC2)cc1]

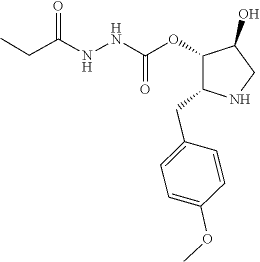 [SMILES: CCC(=O)NNC(=O)O[C@@H]1[C@@H](O)CN[C@@H]1Cc1ccc(OC)cc1]

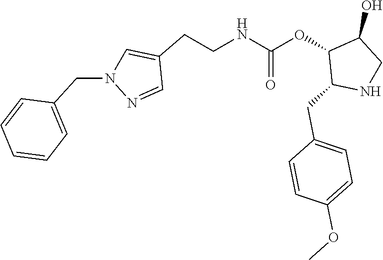 [SMILES: COc1ccc(C[C@H]2NC[C@H](O)[C@H]2OC(=O)NCCc2cnn(Cc3ccccc3)c2)cc1]

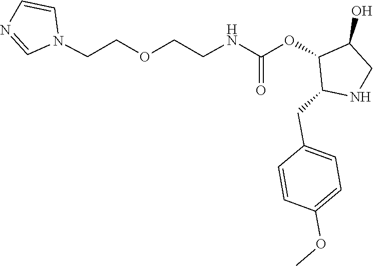 [SMILES: COc1ccc(C[C@H]2NC[C@H](O)[C@H]2OC(=O)NCCOCCn2ccnc2)cc1]

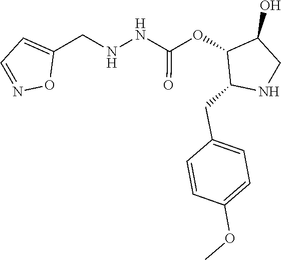 [SMILES: COc1ccc(C[C@H]2NC[C@H](O)[C@H]2OC(=O)NNCc2ccno2)cc1]

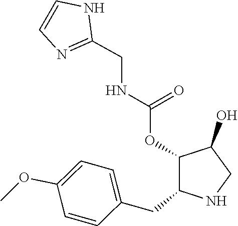 [SMILES: COc1ccc(C[C@H]2NC[C@H](O)[C@H]2OC(=O)NCc2ncc[nH]2)cc1]

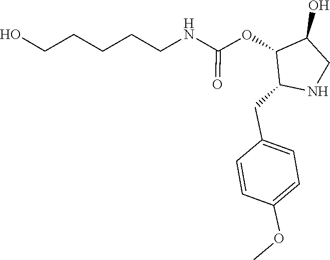 [SMILES: COc1ccc(C[C@H]2NC[C@H](O)[C@H]2OC(=O)NCCCCCO)cc1]

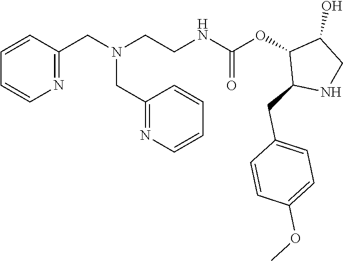 [SMILES: COc1ccc(C[C@@H]2NC[C@@H](O)[C@H]2OC(=O)NCCN(Cc2ccccn2)Cc2ccccn2)cc1]

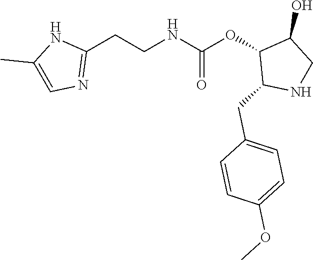 [SMILES: COc1ccc(C[C@H]2NC[C@H](O)[C@H]2OC(=O)NCCc2ncc(C)[nH]2)cc1]

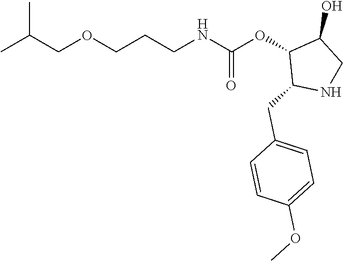 [SMILES: COc1ccc(C[C@H]2NC[C@H](O)[C@H]2OC(=O)NCCCOCC(C)C)cc1]

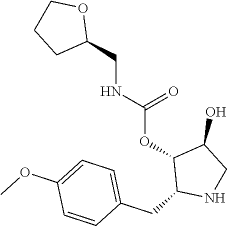 [SMILES: COc1ccc(C[C@H]2NC[C@H](O)[C@H]2OC(=O)NC[C@H]2CCCO2)cc1]

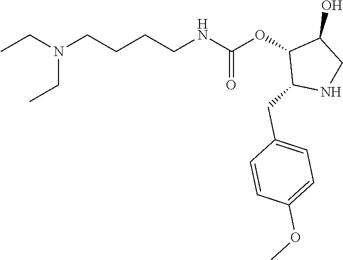 [SMILES: CCN(CC)CCCCNC(=O)O[C@@H]1[C@@H](O)CN[C@@H]1Cc1ccc(OC)cc1]

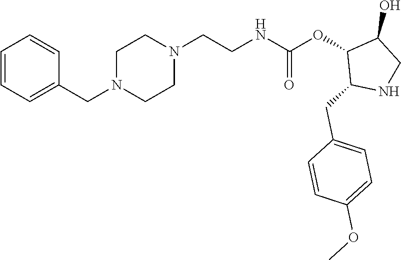 [SMILES: COc1ccc(C[C@H]2NC[C@H](O)[C@H]2OC(=O)NCCN2CCN(Cc3ccccc3)CC2)cc1]

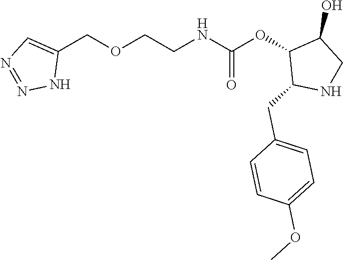 [SMILES: COc1ccc(C[C@H]2NC[C@H](O)[C@H]2OC(=O)NCCOCc2cnn[nH]2)cc1]

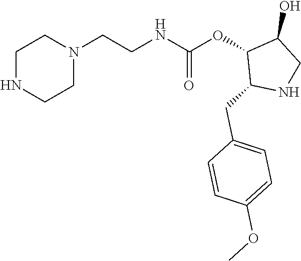 [SMILES: COc1ccc(C[C@H]2NC[C@H](O)[C@H]2OC(=O)NCCN2CCNCC2)cc1]

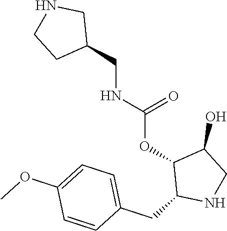 [SMILES: COc1ccc(C[C@H]2NC[C@H](O)[C@H]2OC(=O)NC[C@H]2CCNC2)cc1]

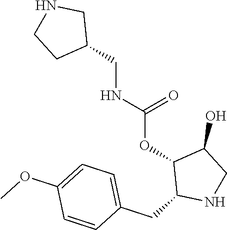 [SMILES: COc1ccc(C[C@H]2NC[C@H](O)[C@H]2OC(=O)NC[C@@H]2CCNC2)cc1]

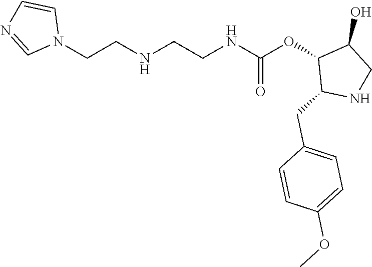 [SMILES: COc1ccc(C[C@H]2NC[C@H](O)[C@H]2OC(=O)NCCNCCn2ccnc2)cc1]

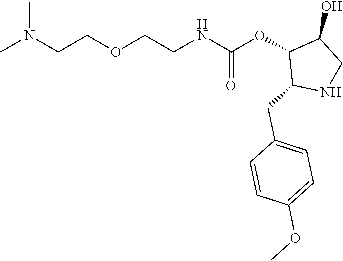 [SMILES: COc1ccc(C[C@H]2NC[C@H](O)[C@H]2OC(=O)NCCOCCN(C)C)cc1]

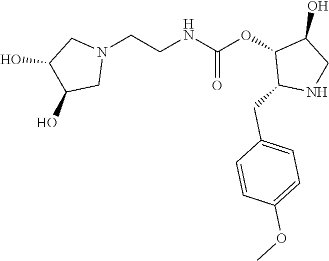 [SMILES: COc1ccc(C[C@H]2NC[C@H](O)[C@H]2OC(=O)NCCN2C[C@@H](O)[C@H](O)C2)cc1]

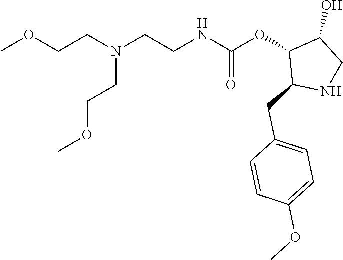 [SMILES: COCCN(CCNC(=O)O[C@@H]1[C@H](O)CN[C@H]1Cc1ccc(OC)cc1)CCOC]

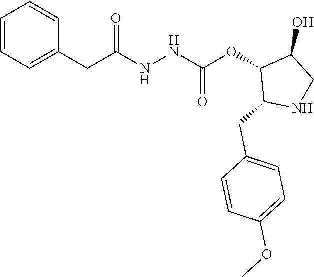 [SMILES: COc1ccc(C[C@H]2NC[C@H](O)[C@H]2OC(=O)NNC(=O)Cc2ccccc2)cc1]

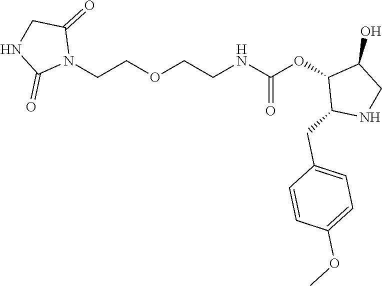 [SMILES: COc1ccc(C[C@H]2NC[C@H](O)[C@H]2OC(=O)NCCOCCN2C(=O)CNC2=O)cc1]

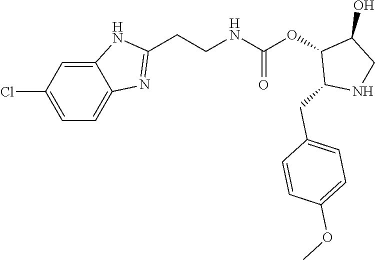 [SMILES: COc1ccc(C[C@H]2NC[C@H](O)[C@H]2OC(=O)NCCc2nc3ccc(Cl)cc3[nH]2)cc1]

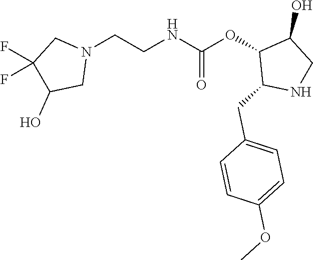 [SMILES: COc1ccc(C[C@H]2NC[C@H](O)[C@H]2OC(=O)NCCN2CC(O)C(F)(F)C2)cc1]

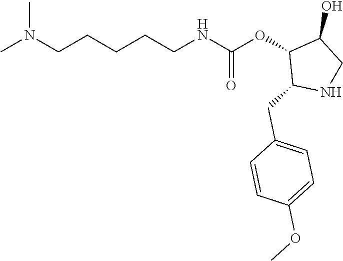 [SMILES: COc1ccc(C[C@H]2NC[C@H](O)[C@H]2OC(=O)NCCCCCN(C)C)cc1]

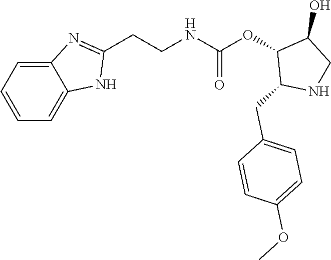 [SMILES: COc1ccc(C[C@H]2NC[C@H](O)[C@H]2OC(=O)NCCc2nc3ccccc3[nH]2)cc1]

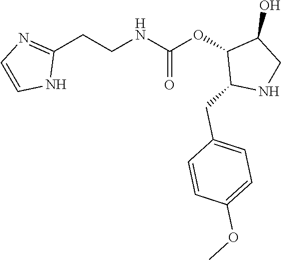 [SMILES: COc1ccc(C[C@H]2NC[C@H](O)[C@H]2OC(=O)NCCc2ncc[nH]2)cc1]

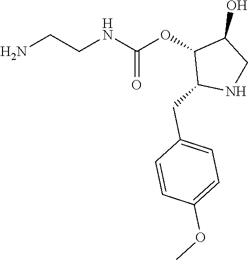 [SMILES: COc1ccc(C[C@H]2NC[C@H](O)[C@H]2OC(=O)NCCN)cc1]

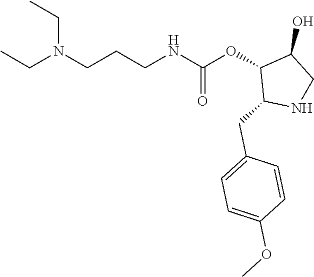 [SMILES: CCN(CC)CCCNC(=O)O[C@@H]1[C@@H](O)CN[C@@H]1Cc1ccc(OC)cc1]

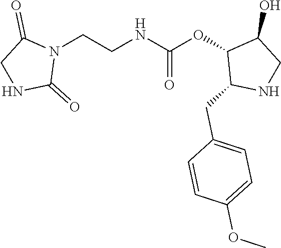 [SMILES: COc1ccc(C[C@H]2NC[C@H](O)[C@H]2OC(=O)NCCN2C(=O)CNC2=O)cc1]

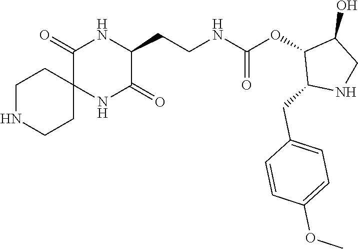 [SMILES: COc1ccc(C[C@H]2NC[C@H](O)[C@H]2OC(=O)NCC[C@@H]2NC(=O)C3(CCNCC3)NC2=O)cc1]

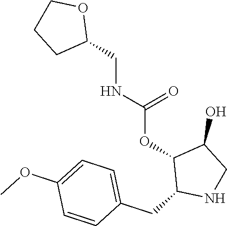 [SMILES: COc1ccc(C[C@H]2NC[C@H](O)[C@H]2OC(=O)NC[C@@H]2CCCO2)cc1]

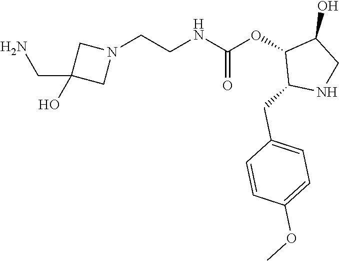 [SMILES: COc1ccc(C[C@H]2NC[C@H](O)[C@H]2OC(=O)NCCN2CC(O)(CN)C2)cc1]

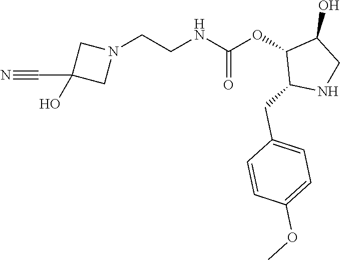 [SMILES: COc1ccc(C[C@H]2NC[C@H](O)[C@H]2OC(=O)NCCN2CC(O)(C#N)C2)cc1]

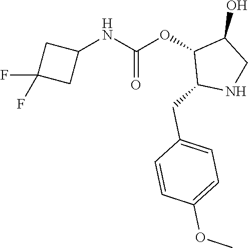 [SMILES: COc1ccc(C[C@H]2NC[C@H](O)[C@H]2OC(=O)NC2CC(F)(F)C2)cc1]

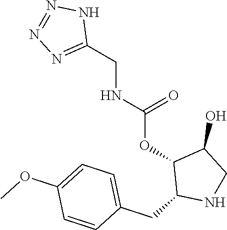 [SMILES: COc1ccc(C[C@H]2NC[C@H](O)[C@H]2OC(=O)NCc2nnn[nH]2)cc1]

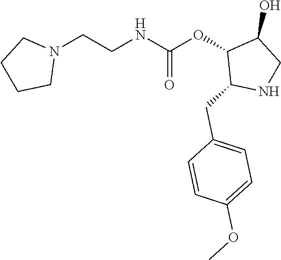 [SMILES: COc1ccc(C[C@H]2NC[C@H](O)[C@H]2OC(=O)NCCN2CCCC2)cc1]

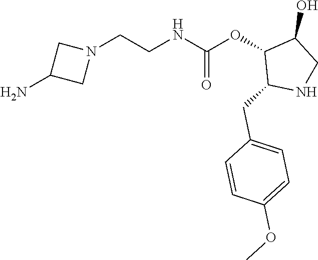 [SMILES: COc1ccc(C[C@H]2NC[C@H](O)[C@H]2OC(=O)NCCN2CC(N)C2)cc1]

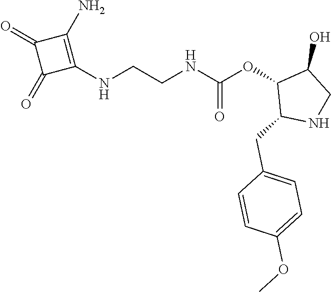 [SMILES: COc1ccc(C[C@H]2NC[C@H](O)[C@H]2OC(=O)NCCNc2c(N)c(=O)c2=O)cc1]

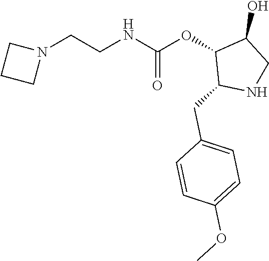 [SMILES: COc1ccc(C[C@H]2NC[C@H](O)[C@H]2OC(=O)NCCN2CCC2)cc1]

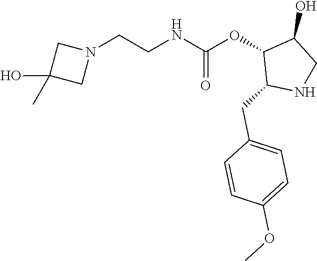 [SMILES: COc1ccc(C[C@H]2NC[C@H](O)[C@H]2OC(=O)NCCN2CC(C)(O)C2)cc1]

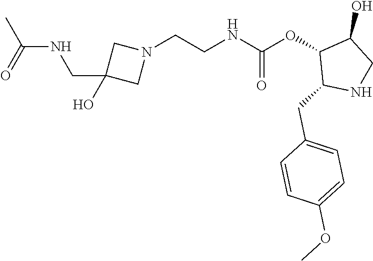 [SMILES: COc1ccc(C[C@H]2NC[C@H](O)[C@H]2OC(=O)NCCN2CC(O)(CNC(C)=O)C2)cc1]

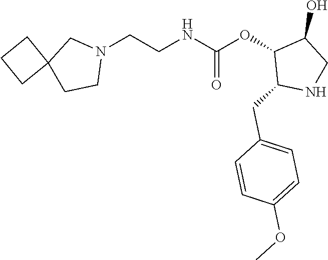 [SMILES: COc1ccc(C[C@H]2NC[C@H](O)[C@H]2OC(=O)NCCN2CCC3(CCC3)C2)cc1]

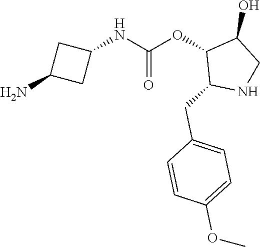 [SMILES: COc1ccc(C[C@H]2NC[C@H](O)[C@H]2OC(=O)N[C@H]2C[C@H](N)C2)cc1]

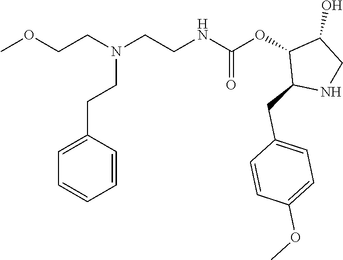 [SMILES: COCCN(CCNC(=O)O[C@@H]1[C@H](O)CN[C@H]1Cc1ccc(OC)cc1)CCc1ccccc1]

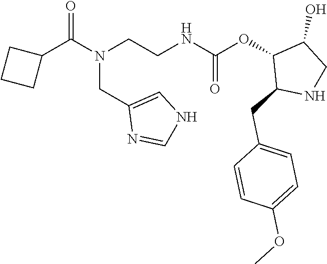 [SMILES: COc1ccc(C[C@@H]2NC[C@@H](O)[C@H]2OC(=O)NCCN(Cc2c[nH]cn2)C(=O)C2CCC2)cc1]

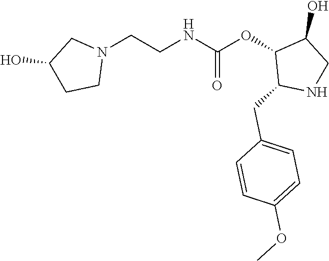 [SMILES: COc1ccc(C[C@H]2NC[C@H](O)[C@H]2OC(=O)NCCN2CC[C@H](O)C2)cc1]

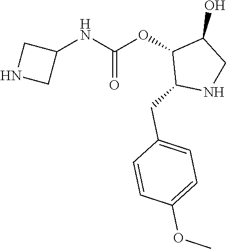 [SMILES: COc1ccc(C[C@H]2NC[C@H](O)[C@H]2OC(=O)NC2CNC2)cc1]

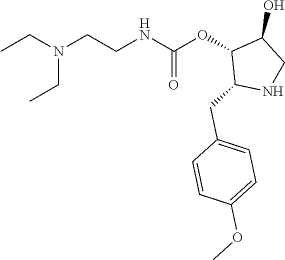 [SMILES: CCN(CC)CCNC(=O)O[C@@H]1[C@@H](O)CN[C@@H]1Cc1ccc(OC)cc1]

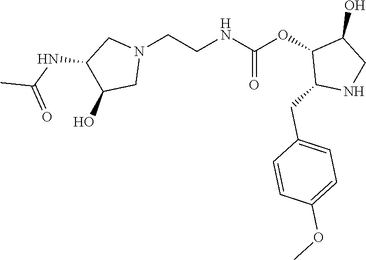 [SMILES: COc1ccc(C[C@H]2NC[C@H](O)[C@H]2OC(=O)NCCN2C[C@@H](O)[C@H](NC(C)=O)C2)cc1]

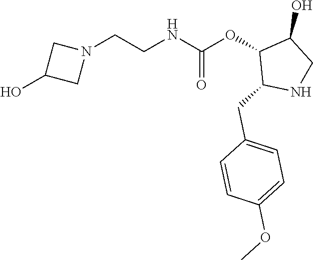 [SMILES: COc1ccc(C[C@H]2NC[C@H](O)[C@H]2OC(=O)NCCN2CC(O)C2)cc1]

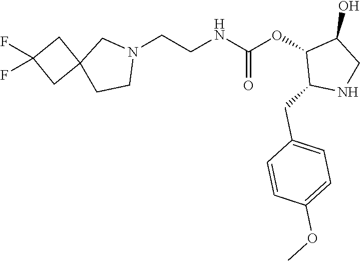 [SMILES: COc1ccc(C[C@H]2NC[C@H](O)[C@H]2OC(=O)NCCN2CCC3(C2)CC(F)(F)C3)cc1]

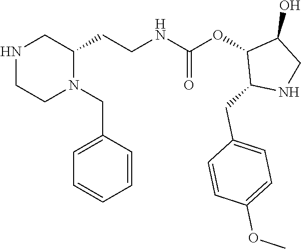 [SMILES: COc1ccc(C[C@H]2NC[C@H](O)[C@H]2OC(=O)NCC[C@H]2CNCCN2Cc2ccccc2)cc1]

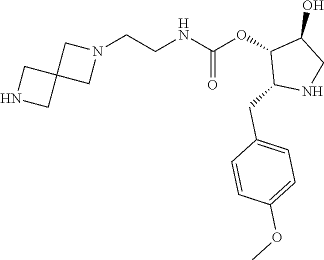 [SMILES: COc1ccc(C[C@H]2NC[C@H](O)[C@H]2OC(=O)NCCN2CC3(CNC3)C2)cc1]

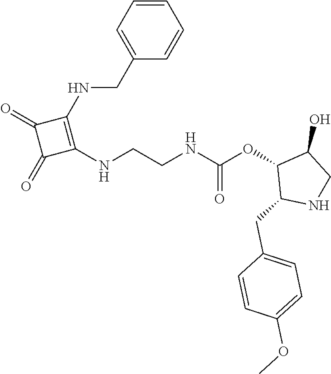 [SMILES: COc1ccc(C[C@H]2NC[C@H](O)[C@H]2OC(=O)NCCNc2c(NCc3ccccc3)c(=O)c2=O)cc1]

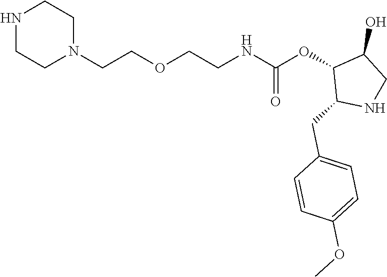 [SMILES: COc1ccc(C[C@H]2NC[C@H](O)[C@H]2OC(=O)NCCOCCN2CCNCC2)cc1]

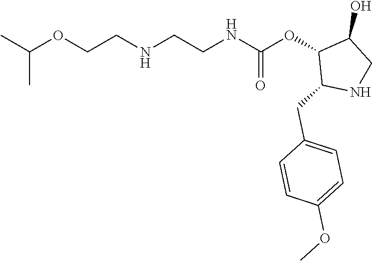 [SMILES: COc1ccc(C[C@H]2NC[C@H](O)[C@H]2OC(=O)NCCNCCOC(C)C)cc1]

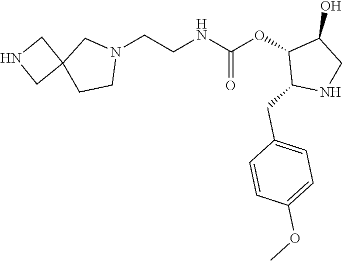 [SMILES: COc1ccc(C[C@H]2NC[C@H](O)[C@H]2OC(=O)NCCN2CCC3(CNC3)C2)cc1]

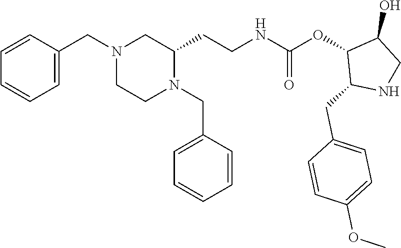 [SMILES: COc1ccc(C[C@H]2NC[C@H](O)[C@H]2OC(=O)NCC[C@H]2CN(Cc3ccccc3)CCN2Cc2ccccc2)cc1]

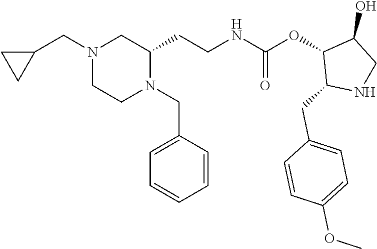 [SMILES: COc1ccc(C[C@H]2NC[C@H](O)[C@H]2OC(=O)NCC[C@H]2CN(CC3CC3)CCN2Cc2ccccc2)cc1]

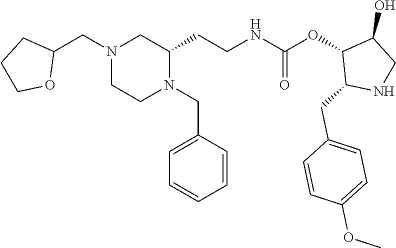 [SMILES: COc1ccc(C[C@H]2NC[C@H](O)[C@H]2OC(=O)NCC[C@H]2CN(CC3CCCO3)CCN2Cc2ccccc2)cc1]

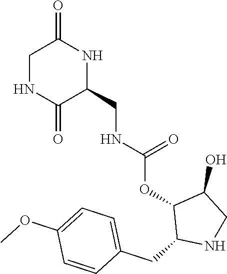 [SMILES: COc1ccc(C[C@H]2NC[C@H](O)[C@H]2OC(=O)NC[C@@H]2NC(=O)CNC2=O)cc1]

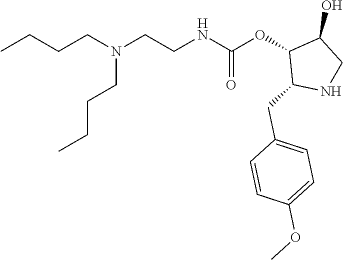 [SMILES: CCCCN(CCCC)CCNC(=O)O[C@@H]1[C@@H](O)CN[C@@H]1Cc1ccc(OC)cc1]

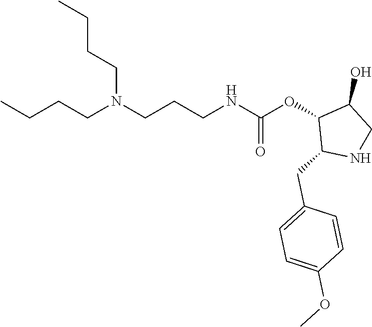 [SMILES: CCCCN(CCCC)CCCNC(=O)O[C@@H]1[C@@H](O)CN[C@@H]1Cc1ccc(OC)cc1]

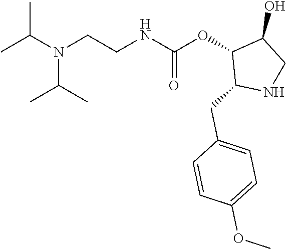 [SMILES: COc1ccc(C[C@H]2NC[C@H](O)[C@H]2OC(=O)NCCN(C(C)C)C(C)C)cc1]

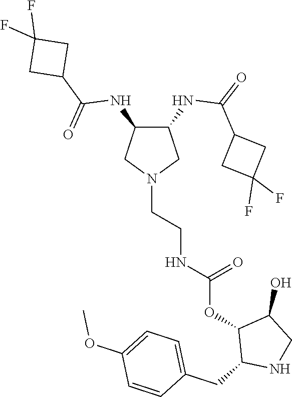 [SMILES: COc1ccc(C[C@H]2NC[C@H](O)[C@H]2OC(=O)NCCN2C[C@@H](NC(=O)C3CC(F)(F)C3)[C@H](NC(=O)C3CC(F)(F)C3)C2)cc1]

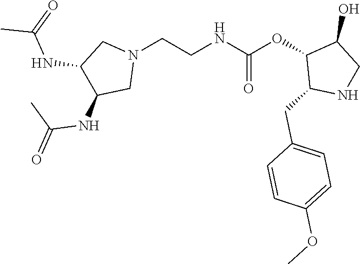 [SMILES: COc1ccc(C[C@H]2NC[C@H](O)[C@H]2OC(=O)NCCN2C[C@@H](NC(C)=O)[C@H](NC(C)=O)C2)cc1]

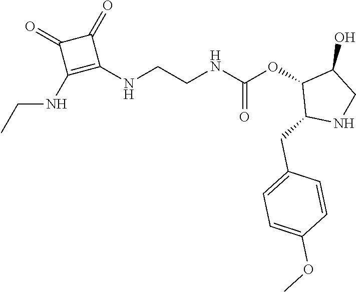 [SMILES: CCNc1c(NCCNC(=O)O[C@@H]2[C@@H](O)CN[C@@H]2Cc2ccc(OC)cc2)c(=O)c1=O]

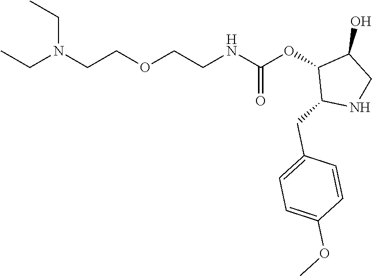 [SMILES: CCN(CC)CCOCCNC(=O)O[C@@H]1[C@@H](O)CN[C@@H]1Cc1ccc(OC)cc1]

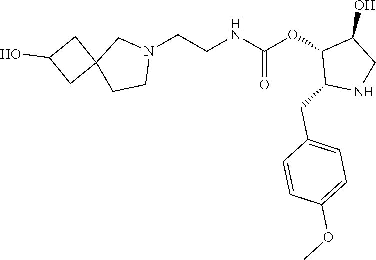 [SMILES: COc1ccc(C[C@H]2NC[C@H](O)[C@H]2OC(=O)NCCN2CCC3(CC(O)C3)C2)cc1]

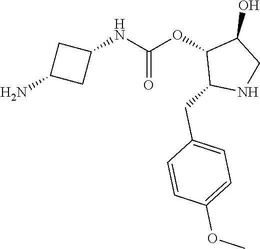 [SMILES: COc1ccc(C[C@H]2NC[C@H](O)[C@H]2OC(=O)N[C@H]2C[C@@H](N)C2)cc1]

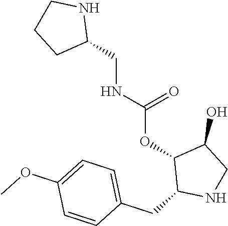 [SMILES: COc1ccc(C[C@H]2NC[C@H](O)[C@H]2OC(=O)NC[C@@H]2CCCN2)cc1]

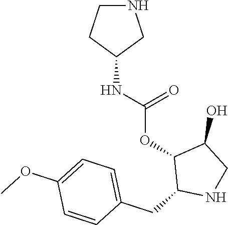 [SMILES: COc1ccc(C[C@H]2NC[C@H](O)[C@H]2OC(=O)N[C@@H]2CCNC2)cc1]

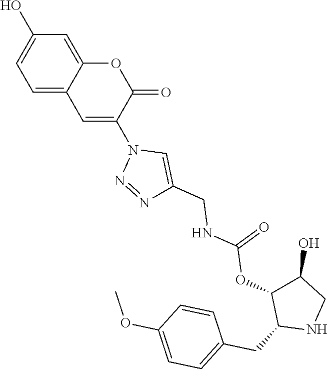 [SMILES: COc1ccc(C[C@H]2NC[C@H](O)[C@H]2OC(=O)NCc2cn(-c3cc4ccc(O)cc4oc3=O)nn2)cc1]

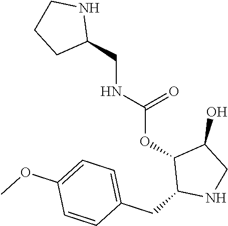 [SMILES: COc1ccc(C[C@H]2NC[C@H](O)[C@H]2OC(=O)NC[C@H]2CCCN2)cc1]

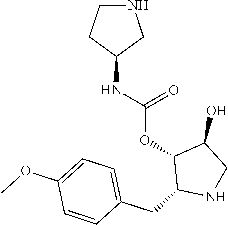 [SMILES: COc1ccc(C[C@H]2NC[C@H](O)[C@H]2OC(=O)N[C@H]2CCNC2)cc1]

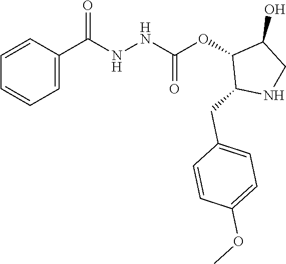 [SMILES: COc1ccc(C[C@H]2NC[C@H](O)[C@H]2OC(=O)NNC(=O)c2ccccc2)cc1]

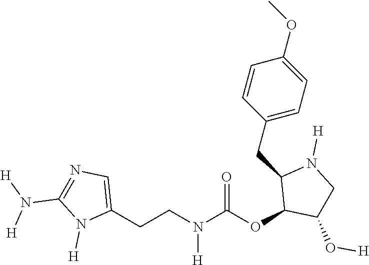 [SMILES: COc1ccc(C[C@H]2NC[C@H](O)[C@H]2OC(=O)NCCc2cnc(N)[nH]2)cc1]

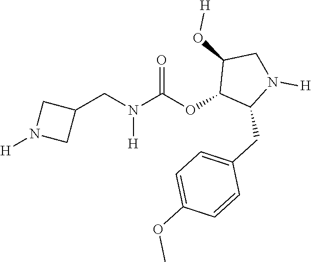 [SMILES: COc1ccc(C[C@H]2NC[C@H](O)[C@H]2OC(=O)NCC2CNC2)cc1]

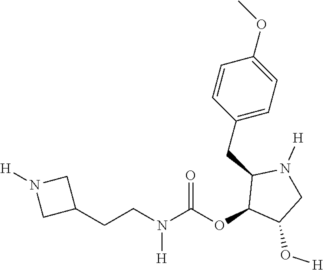 [SMILES: COc1ccc(C[C@H]2NC[C@H](O)[C@H]2OC(=O)NCCC2CNC2)cc1]

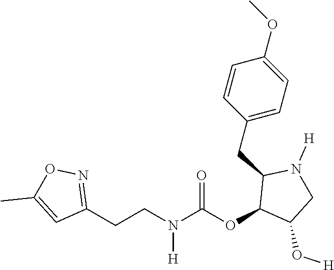 [SMILES: COc1ccc(C[C@H]2NC[C@H](O)[C@H]2OC(=O)NCCc2cc(C)on2)cc1]

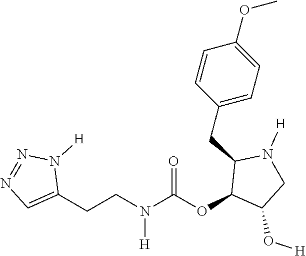 [SMILES: COc1ccc(C[C@H]2NC[C@H](O)[C@H]2OC(=O)NCCc2cnn[nH]2)cc1]

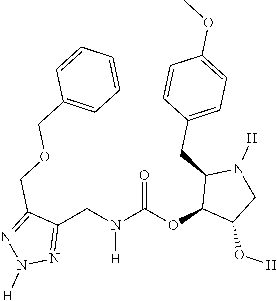 [SMILES: COc1ccc(C[C@H]2NC[C@H](O)[C@H]2OC(=O)NCc2n[nH]nc2COCc2ccccc2)cc1]

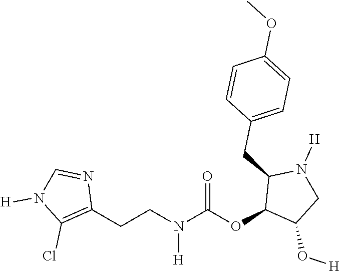 [SMILES: COc1ccc(C[C@H]2NC[C@H](O)[C@H]2OC(=O)NCCc2nc[nH]c2Cl)cc1]

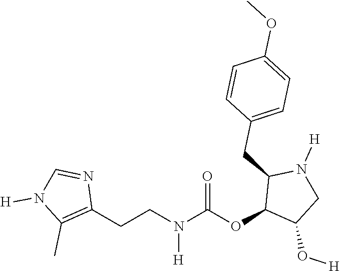 [SMILES: COc1ccc(C[C@H]2NC[C@H](O)[C@H]2OC(=O)NCCc2nc[nH]c2C)cc1]

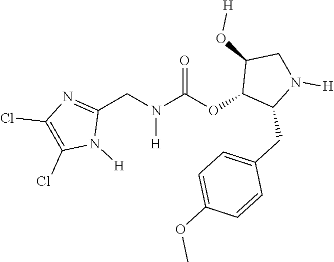 [SMILES: COc1ccc(C[C@H]2NC[C@H](O)[C@H]2OC(=O)NCc2nc(Cl)c(Cl)[nH]2)cc1]

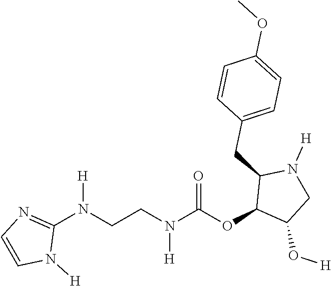 [SMILES: COc1ccc(C[C@H]2NC[C@H](O)[C@H]2OC(=O)NCCNc2ncc[nH]2)cc1]

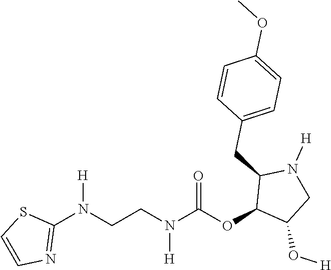 [SMILES: COc1ccc(C[C@H]2NC[C@H](O)[C@H]2OC(=O)NCCNc2nccs2)cc1]

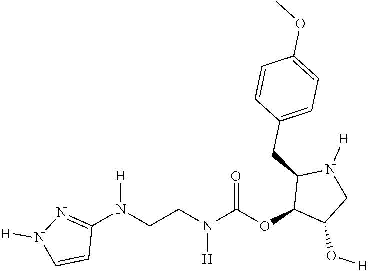 [SMILES: COc1ccc(C[C@H]2NC[C@H](O)[C@H]2OC(=O)NCCNc2cc[nH]n2)cc1]

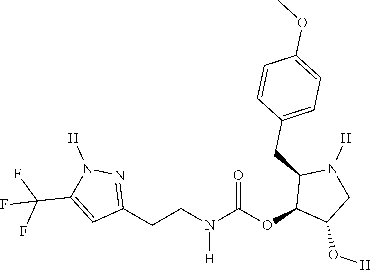 [SMILES: COc1ccc(C[C@H]2NC[C@H](O)[C@H]2OC(=O)NCCc2cc(C(F)(F)F)[nH]n2)cc1]

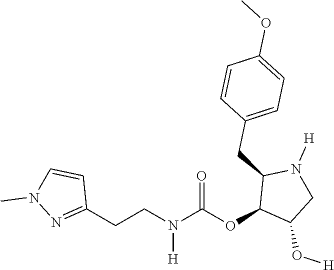 [SMILES: COc1ccc(C[C@H]2NC[C@H](O)[C@H]2OC(=O)NCCc2ccn(C)n2)cc1]

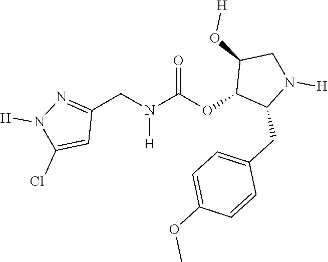 [SMILES: COc1ccc(C[C@H]2NC[C@H](O)[C@H]2OC(=O)NCc2cc(Cl)[nH]n2)cc1]

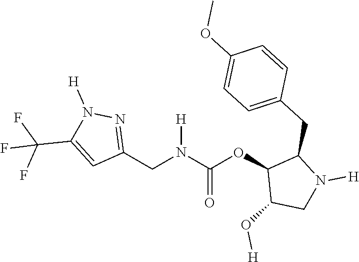 [SMILES: COc1ccc(C[C@H]2NC[C@H](O)[C@H]2OC(=O)NCc2cc(C(F)(F)F)[nH]n2)cc1]

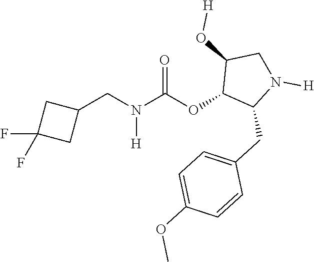 [SMILES: COc1ccc(C[C@H]2NC[C@H](O)[C@H]2OC(=O)NCC2CC(F)(F)C2)cc1]

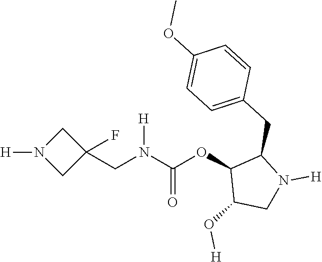 [SMILES: COc1ccc(C[C@H]2NC[C@H](O)[C@H]2OC(=O)NCC2(F)CNC2)cc1]

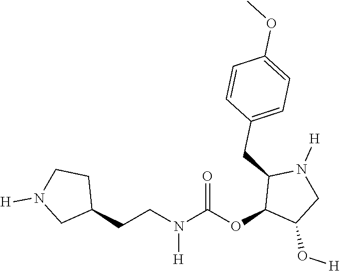 [SMILES: COc1ccc(C[C@H]2NC[C@H](O)[C@H]2OC(=O)NCC[C@@H]2CCNC2)cc1]

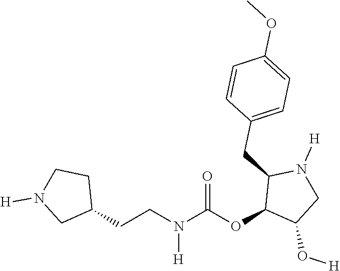 [SMILES: COc1ccc(C[C@H]2NC[C@H](O)[C@H]2OC(=O)NCC[C@H]2CCNC2)cc1]

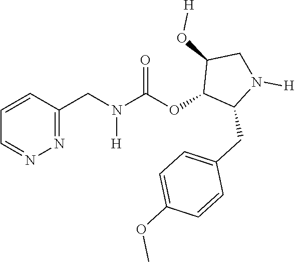 [SMILES: COc1ccc(C[C@H]2NC[C@H](O)[C@H]2OC(=O)NCc2cccnn2)cc1]

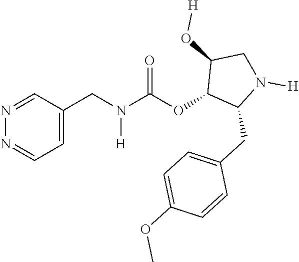 [SMILES: COc1ccc(C[C@H]2NC[C@H](O)[C@H]2OC(=O)NCc2ccnnc2)cc1]

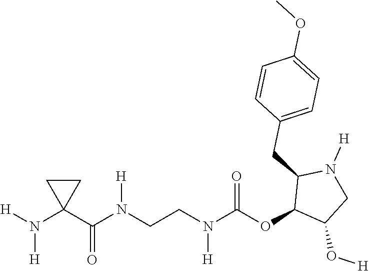 [SMILES: COc1ccc(C[C@H]2NC[C@H](O)[C@H]2OC(=O)NCCNC(=O)C2(N)CC2)cc1]

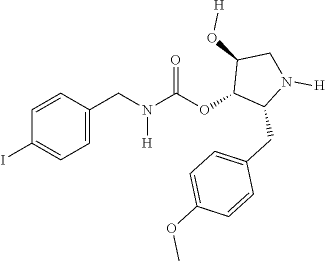 [SMILES: COc1ccc(C[C@H]2NC[C@H](O)[C@H]2OC(=O)NCc2ccc(I)cc2)cc1]

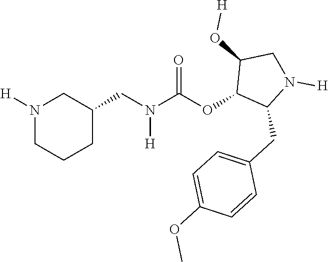 [SMILES: COc1ccc(C[C@H]2NC[C@H](O)[C@H]2OC(=O)NC[C@@H]2CCCNC2)cc1]

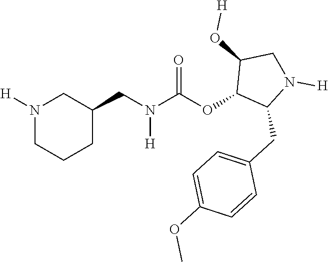 [SMILES: COc1ccc(C[C@H]2NC[C@H](O)[C@H]2OC(=O)NC[C@H]2CCCNC2)cc1]

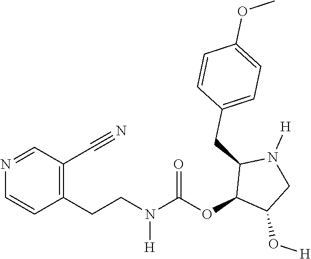 [SMILES: COc1ccc(C[C@H]2NC[C@H](O)[C@H]2OC(=O)NCCc2ccncc2C#N)cc1]

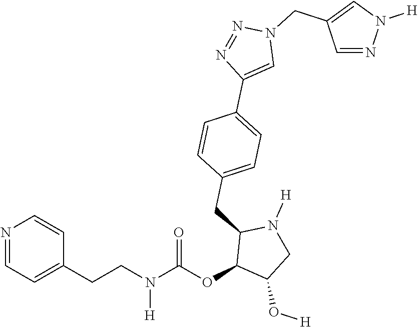 [SMILES: O=C(NCCc1ccncc1)O[C@@H]1[C@@H](O)CN[C@@H]1Cc1ccc(-c2cn(Cc3cn[nH]c3)nn2)cc1]

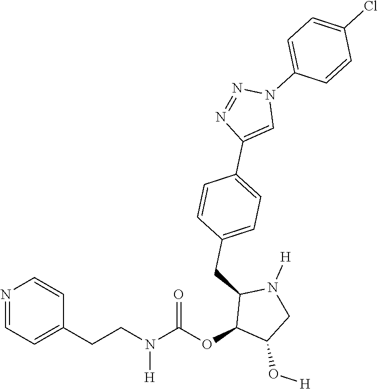 [SMILES: O=C(NCCc1ccncc1)O[C@@H]1[C@@H](O)CN[C@@H]1Cc1ccc(-c2cn(-c3ccc(Cl)cc3)nn2)cc1]

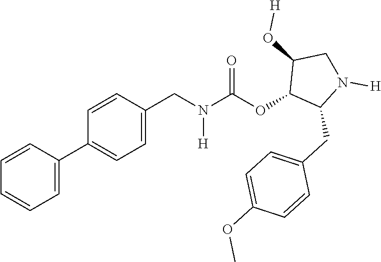 [SMILES: COc1ccc(C[C@H]2NC[C@H](O)[C@H]2OC(=O)NCc2ccc(-c3ccccc3)cc2)cc1]

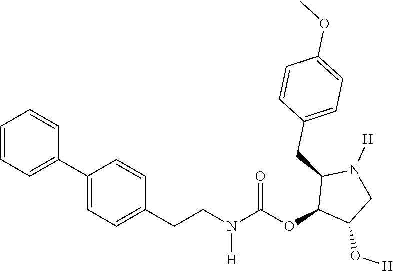 [SMILES: COc1ccc(C[C@H]2NC[C@H](O)[C@H]2OC(=O)NCCc2ccc(-c3ccccc3)cc2)cc1]

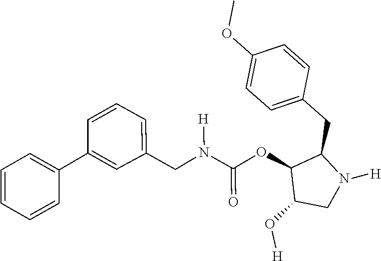 [SMILES: COc1ccc(C[C@H]2NC[C@H](O)[C@H]2OC(=O)NCc2cccc(-c3ccccc3)c2)cc1]

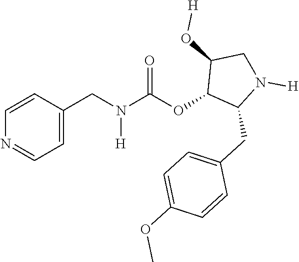 [SMILES: COc1ccc(C[C@H]2NC[C@H](O)[C@H]2OC(=O)NCc2ccncc2)cc1]

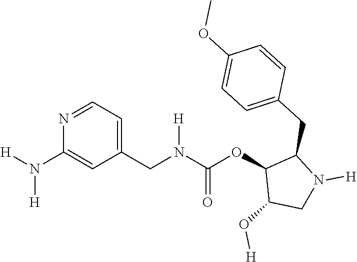 [SMILES: COc1ccc(C[C@H]2NC[C@H](O)[C@H]2OC(=O)NCc2ccnc(N)c2)cc1]

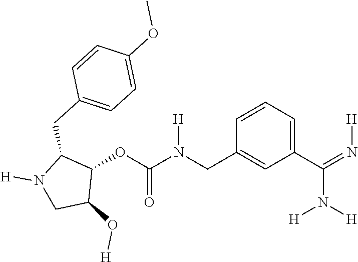 [SMILES: [H]/N=C(/N)c1cccc(CNC(=O)O[C@@H]2[C@@H](O)CN[C@@H]2Cc2ccc(OC)cc2)c1]

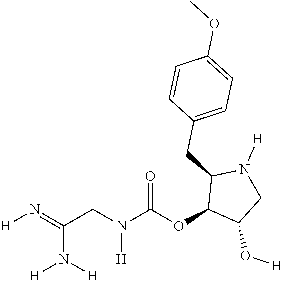 [SMILES: [H]/N=C(\N)CNC(=O)O[C@@H]1[C@@H](O)CN[C@@H]1Cc1ccc(OC)cc1]

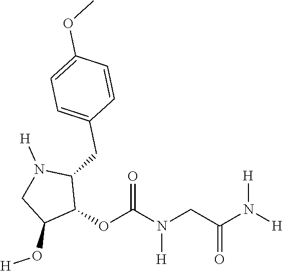 [SMILES: COc1ccc(C[C@H]2NC[C@H](O)[C@H]2OC(=O)NCC(N)=O)cc1]

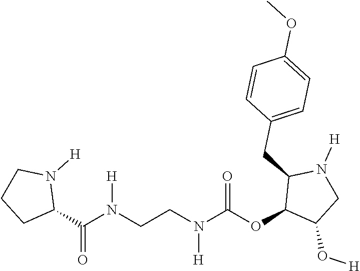 [SMILES: COc1ccc(C[C@H]2NC[C@H](O)[C@H]2OC(=O)NCCNC(=O)[C@@H]2CCCN2)cc1]

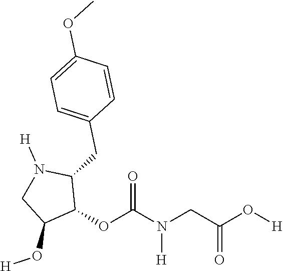 [SMILES: COc1ccc(C[C@H]2NC[C@H](O)[C@H]2OC(=O)NCC(=O)O)cc1]

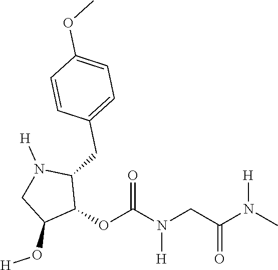 [SMILES: CNC(=O)CNC(=O)O[C@@H]1[C@@H](O)CN[C@@H]1Cc1ccc(OC)cc1]

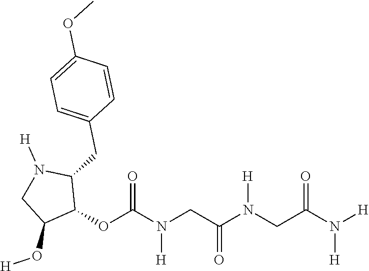 [SMILES: COc1ccc(C[C@H]2NC[C@H](O)[C@H]2OC(=O)NCC(=O)NCC(N)=O)cc1]

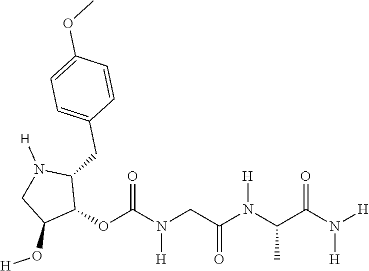 [SMILES: COc1ccc(C[C@H]2NC[C@H](O)[C@H]2OC(=O)NCC(=O)N[C@@H](C)C(N)=O)cc1]

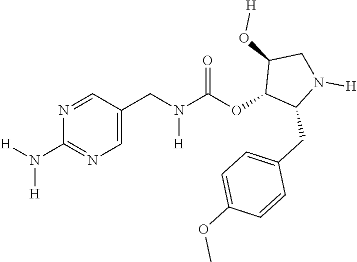 [SMILES: COc1ccc(C[C@H]2NC[C@H](O)[C@H]2OC(=O)NCc2cnc(N)nc2)cc1]

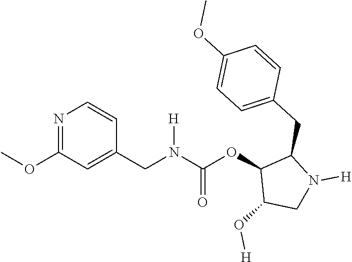 [SMILES: COc1ccc(C[C@H]2NC[C@H](O)[C@H]2OC(=O)NCc2ccnc(OC)c2)cc1]

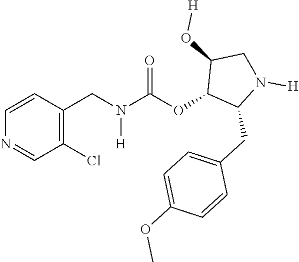 [SMILES: COc1ccc(C[C@H]2NC[C@H](O)[C@H]2OC(=O)NCc2ccncc2Cl)cc1]

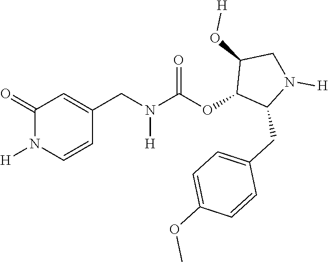 [SMILES: COc1ccc(C[C@H]2NC[C@H](O)[C@H]2OC(=O)NCc2cc[nH]c(=O)c2)cc1]

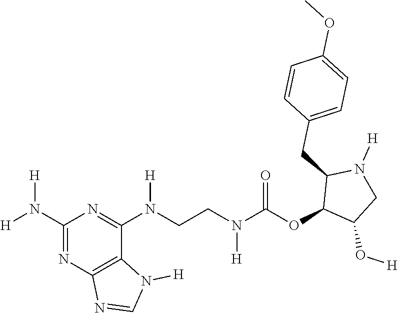 [SMILES: COc1ccc(C[C@H]2NC[C@H](O)[C@H]2OC(=O)NCCNc2nc(N)nc3nc[nH]c23)cc1]

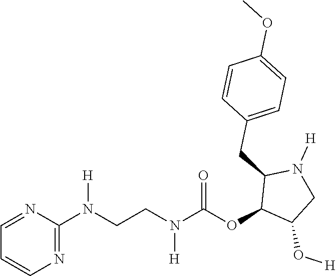 [SMILES: COc1ccc(C[C@H]2NC[C@H](O)[C@H]2OC(=O)NCCNc2ncccn2)cc1]

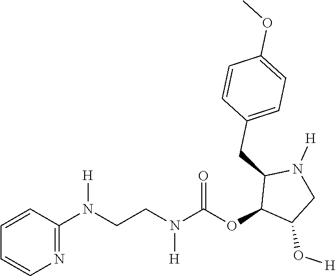 [SMILES: COc1ccc(C[C@H]2NC[C@H](O)[C@H]2OC(=O)NCCNc2ccccn2)cc1]

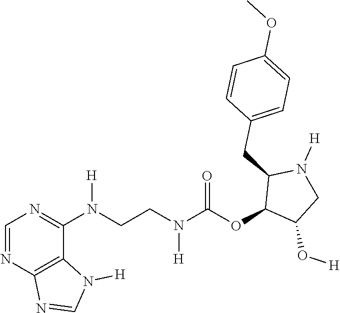 [SMILES: COc1ccc(C[C@H]2NC[C@H](O)[C@H]2OC(=O)NCCNc2ncnc3nc[nH]c23)cc1]

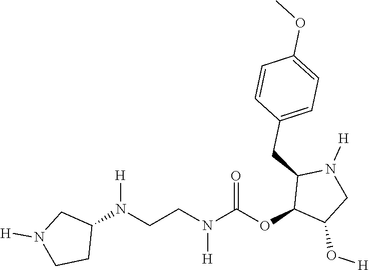 [SMILES: COc1ccc(C[C@H]2NC[C@H](O)[C@H]2OC(=O)NCCN[C@@H]2CCNC2)cc1]

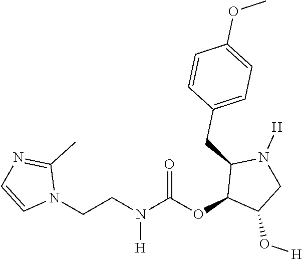 [SMILES: COc1ccc(C[C@H]2NC[C@H](O)[C@H]2OC(=O)NCCn2ccnc2C)cc1]

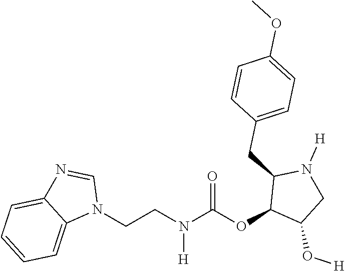 [SMILES: COc1ccc(C[C@H]2NC[C@H](O)[C@H]2OC(=O)NCCn2cnc3ccccc32)cc1]

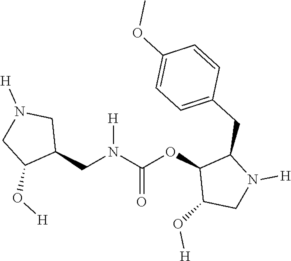 [SMILES: COc1ccc(C[C@H]2NC[C@H](O)[C@H]2OC(=O)NC[C@@H]2CNC[C@H]2O)cc1]

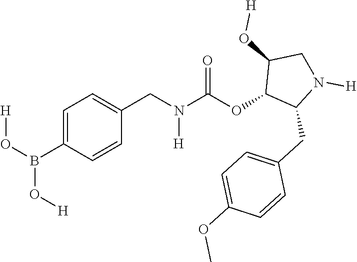 [SMILES: COc1ccc(C[C@H]2NC[C@H](O)[C@H]2OC(=O)NCc2ccc(B(O)O)cc2)cc1]

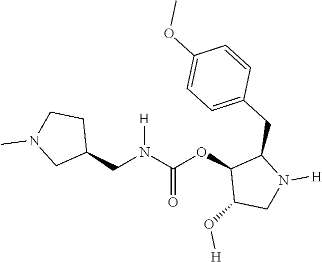 [SMILES: COc1ccc(C[C@H]2NC[C@H](O)[C@H]2OC(=O)NC[C@@H]2CCN(C)C2)cc1]

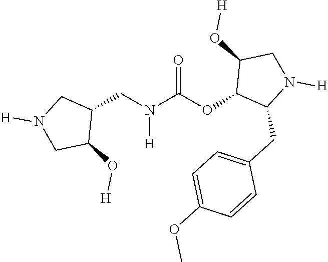 [SMILES: COc1ccc(C[C@H]2NC[C@H](O)[C@H]2OC(=O)NC[C@H]2CNC[C@@H]2O)cc1]

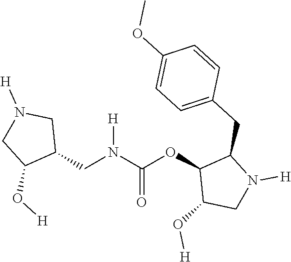 [SMILES: COc1ccc(C[C@H]2NC[C@H](O)[C@H]2OC(=O)NC[C@H]2CNC[C@H]2O)cc1]

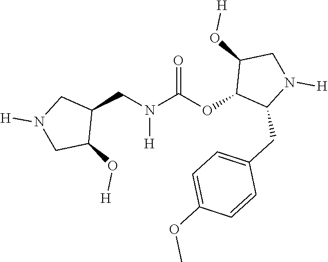 [SMILES: COc1ccc(C[C@H]2NC[C@H](O)[C@H]2OC(=O)NC[C@@H]2CNC[C@@H]2O)cc1]

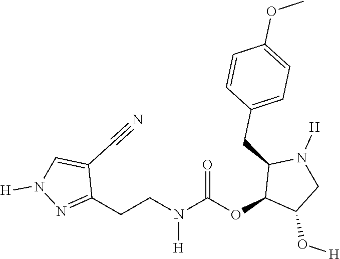 [SMILES: COc1ccc(C[C@H]2NC[C@H](O)[C@H]2OC(=O)NCCc2n[nH]cc2C#N)cc1]

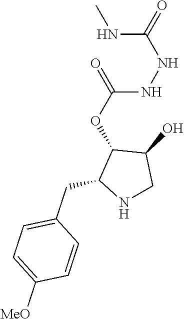 [SMILES: CNC(=O)NNC(=O)O[C@@H]1[C@@H](O)CN[C@@H]1Cc1ccc(OC)cc1]

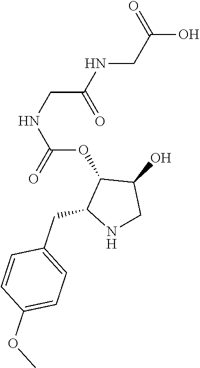 [SMILES: COc1ccc(C[C@H]2NC[C@H](O)[C@H]2OC(=O)NCC(=O)NCC(=O)O)cc1]

TABLE 1B
Additional Compounds of Formula (I)

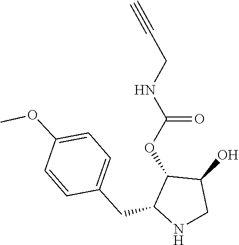 [SMILES: C#CCNC(=O)O[C@@H]1[C@@H](O)CN[C@@H]1Cc1ccc(OC)cc1]

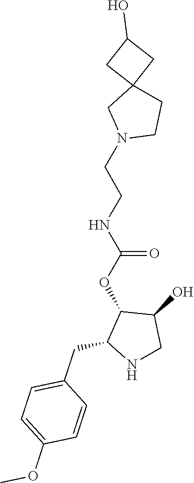 [SMILES: COc1ccc(C[C@H]2NC[C@H](O)[C@H]2OC(=O)NCCN2CCC3(CC(O)C3)C2)cc1]

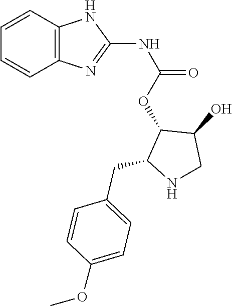 [SMILES: COc1ccc(C[C@H]2NC[C@H](O)[C@H]2OC(=O)Nc2nc3ccccc3[nH]2)cc1]

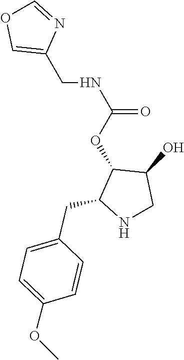 [SMILES: COc1ccc(C[C@H]2NC[C@H](O)[C@H]2OC(=O)NCc2cocn2)cc1]

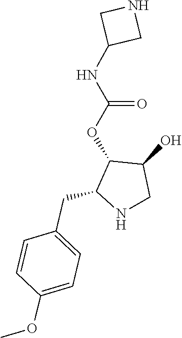 [SMILES: COc1ccc(C[C@H]2NC[C@H](O)[C@H]2OC(=O)NC2CNC2)cc1]

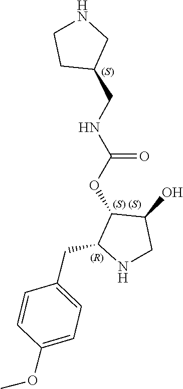

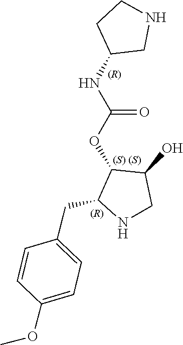

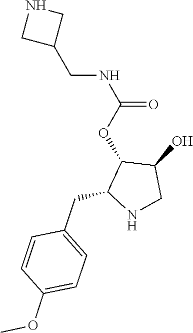 [SMILES: COc1ccc(C[C@H]2NC[C@H](O)[C@H]2OC(=O)NCC2CNC2)cc1]

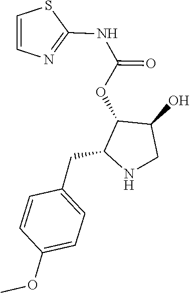 [SMILES: COc1ccc(C[C@H]2NC[C@H](O)[C@H]2OC(=O)Nc2nccs2)cc1]

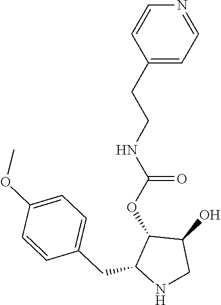 [SMILES: COc1ccc(C[C@H]2NC[C@H](O)[C@H]2OC(=O)NCCc2ccncc2)cc1]

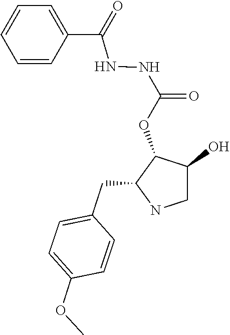

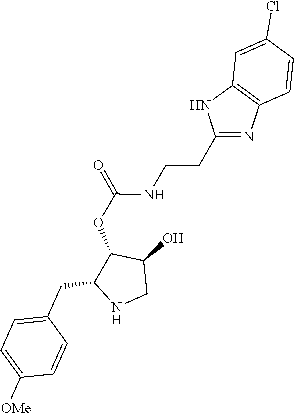 [SMILES: COc1ccc(C[C@H]2NC[C@H](O)[C@H]2OC(=O)NCCc2nc3ccc(Cl)cc3[nH]2)cc1]

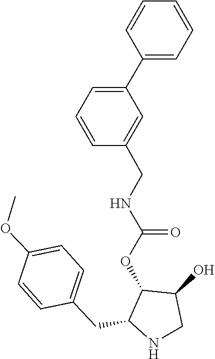 [SMILES: COc1ccc(C[C@H]2NC[C@H](O)[C@H]2OC(=O)NCc2cccc(-c3ccccc3)c2)cc1]

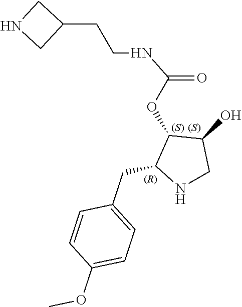

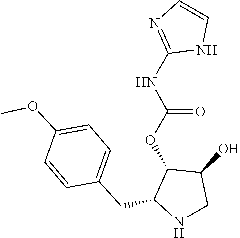 [SMILES: COc1ccc(C[C@H]2NC[C@H](O)[C@H]2OC(=O)Nc2ncc[nH]2)cc1]

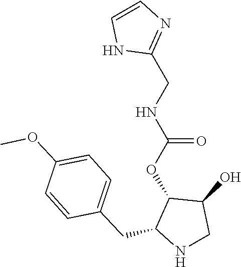 [SMILES: COc1ccc(C[C@H]2NC[C@H](O)[C@H]2OC(=O)NCc2ncc[nH]2)cc1]

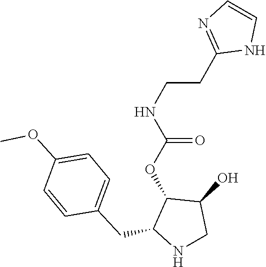 [SMILES: COc1ccc(C[C@H]2NC[C@H](O)[C@H]2OC(=O)NCCc2ncc[nH]2)cc1]

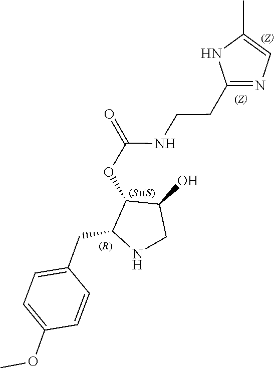

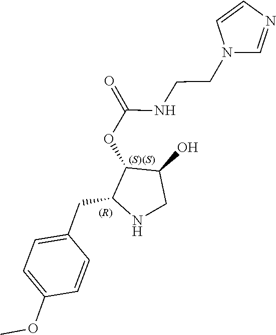

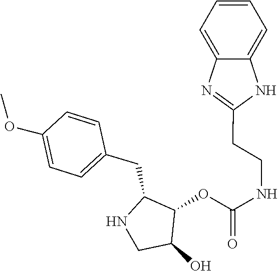 [SMILES: COc1ccc(C[C@H]2NC[C@H](O)[C@H]2OC(=O)NCCc2nc3ccccc3[nH]2)cc1]

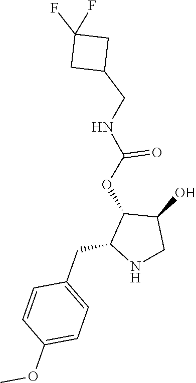 [SMILES: COc1ccc(C[C@H]2NC[C@H](O)[C@H]2OC(=O)NCC2CC(F)(F)C2)cc1]

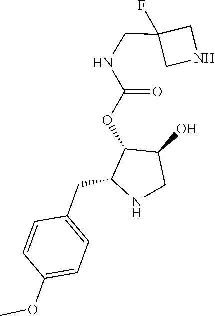 [SMILES: COc1ccc(C[C@H]2NC[C@H](O)[C@H]2OC(=O)NCC2(F)CNC2)cc1]

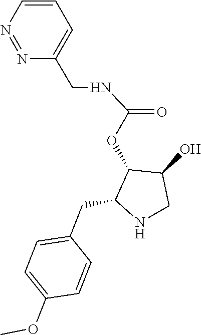 [SMILES: COc1ccc(C[C@H]2NC[C@H](O)[C@H]2OC(=O)NCc2cccnn2)cc1]

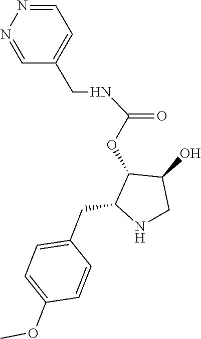 [SMILES: COc1ccc(C[C@H]2NC[C@H](O)[C@H]2OC(=O)NCc2ccnnc2)cc1]

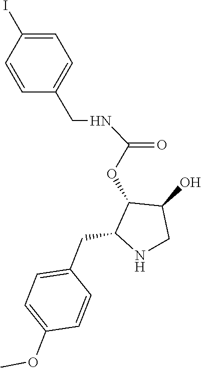 [SMILES: COc1ccc(C[C@H]2NC[C@H](O)[C@H]2OC(=O)NCc2ccc(I)cc2)cc1]

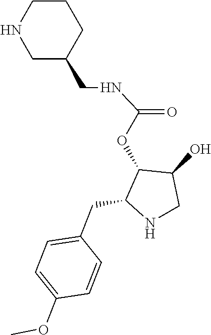 [SMILES: COc1ccc(C[C@H]2NC[C@H](O)[C@H]2OC(=O)NC[C@@H]2CCCNC2)cc1]

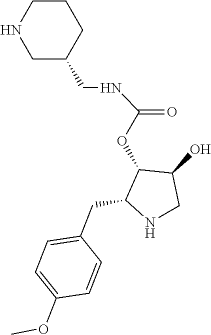 [SMILES: COc1ccc(C[C@H]2NC[C@H](O)[C@H]2OC(=O)NC[C@H]2CCCNC2)cc1]

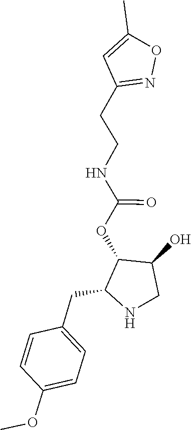 [SMILES: COc1ccc(C[C@H]2NC[C@H](O)[C@H]2OC(=O)NCCc2cc(C)on2)cc1]

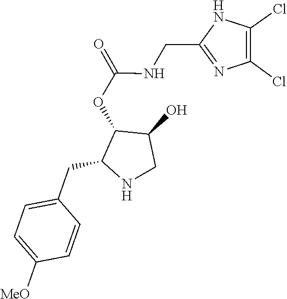 [SMILES: COc1ccc(C[C@H]2NC[C@H](O)[C@H]2OC(=O)NCc2nc(Cl)c(Cl)[nH]2)cc1]

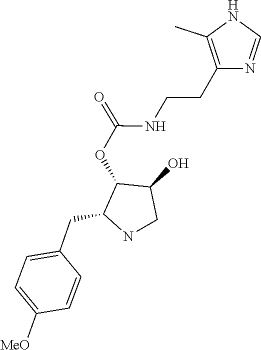

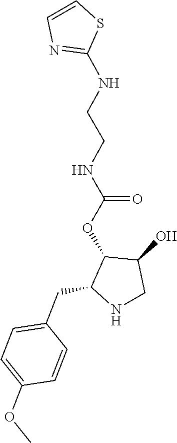 [SMILES: COc1ccc(C[C@H]2NC[C@H](O)[C@H]2OC(=O)NCCNc2nccs2)cc1]

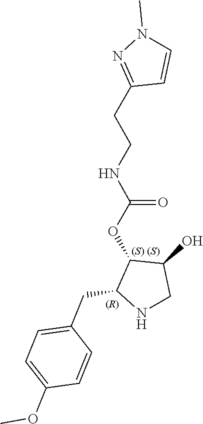

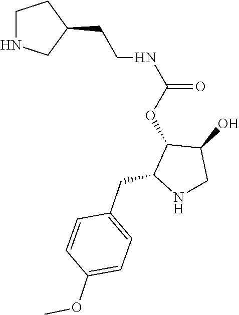 [SMILES: COc1ccc(C[C@H]2NC[C@H](O)[C@H]2OC(=O)NCC[C@@H]2CCNC2)cc1]

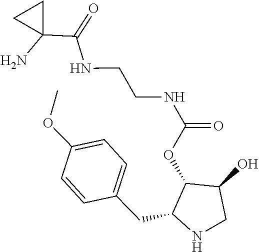 [SMILES: COc1ccc(C[C@H]2NC[C@H](O)[C@H]2OC(=O)NCCNC(=O)C2(N)CC2)cc1]

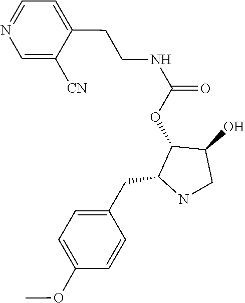

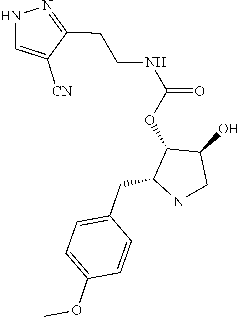

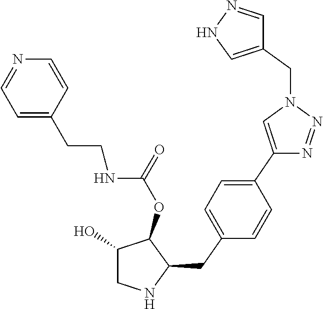 [SMILES: O=C(NCCc1ccncc1)O[C@@H]1[C@@H](O)CN[C@@H]1Cc1ccc(-c2cn(Cc3cn[nH]c3)nn2)cc1]

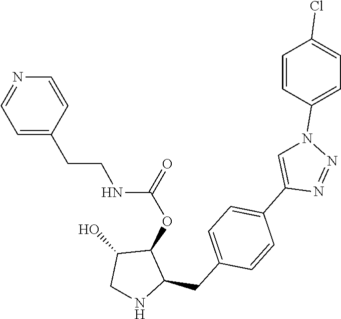 [SMILES: O=C(NCCc1ccncc1)O[C@@H]1[C@@H](O)CN[C@@H]1Cc1ccc(-c2cn(-c3ccc(Cl)cc3)nn2)cc1]

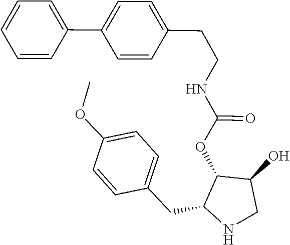 [SMILES: COc1ccc(C[C@H]2NC[C@H](O)[C@H]2OC(=O)NCCc2ccc(-c3ccccc3)cc2)cc1]

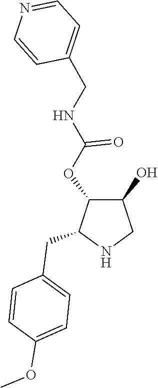 [SMILES: COc1ccc(C[C@H]2NC[C@H](O)[C@H]2OC(=O)NCc2ccncc2)cc1]

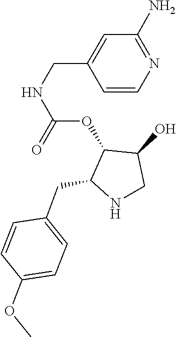 [SMILES: COc1ccc(C[C@H]2NC[C@H](O)[C@H]2OC(=O)NCc2ccnc(N)c2)cc1]

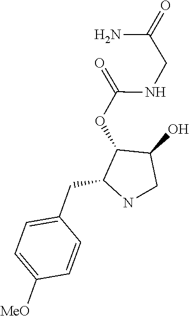

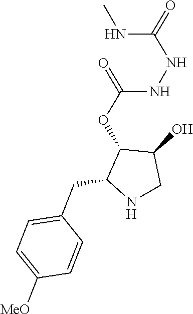 [SMILES: CNC(=O)NNC(=O)O[C@@H]1[C@@H](O)CN[C@@H]1Cc1ccc(OC)cc1]

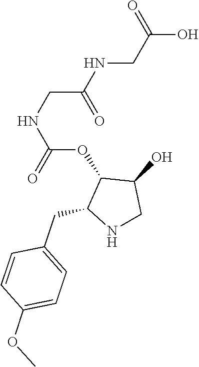 [SMILES: COc1ccc(C[C@H]2NC[C@H](O)[C@H]2OC(=O)NCC(=O)NCC(=O)O)cc1]

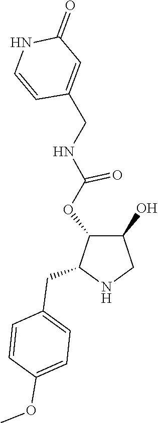 [SMILES: COc1ccc(C[C@H]2NC[C@H](O)[C@H]2OC(=O)NCc2cc[nH]c(=O)c2)cc1]

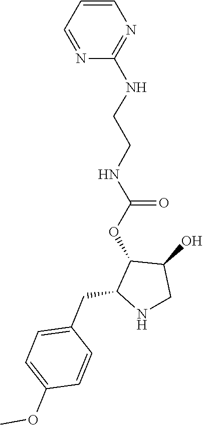 [SMILES: COc1ccc(C[C@H]2NC[C@H](O)[C@H]2OC(=O)NCCNc2ncccn2)cc1]

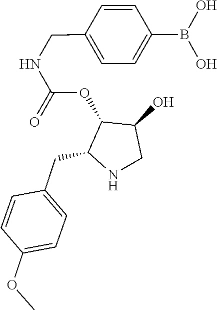 [SMILES: COc1ccc(C[C@H]2NC[C@H](O)[C@H]2OC(=O)NCc2ccc(B(O)O)cc2)cc1]

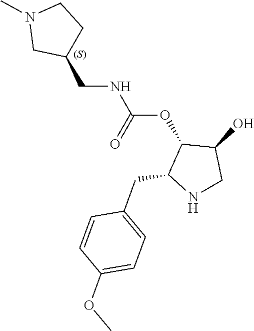

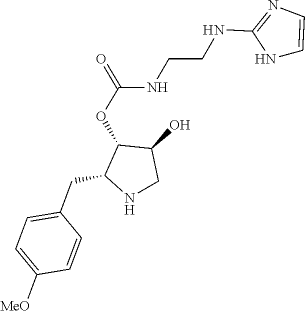 [SMILES: COc1ccc(C[C@H]2NC[C@H](O)[C@H]2OC(=O)NCCNc2ncc[nH]2)cc1]

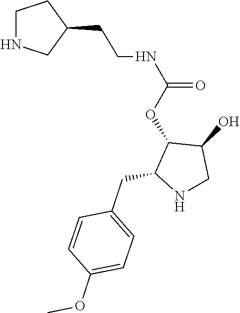 [SMILES: COc1ccc(C[C@H]2NC[C@H](O)[C@H]2OC(=O)NCC[C@@H]2CCNC2)cc1]

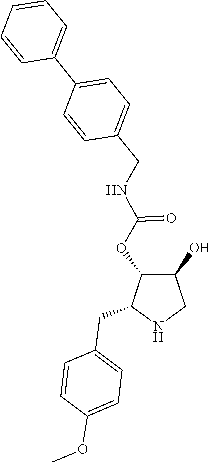 [SMILES: COc1ccc(C[C@H]2NC[C@H](O)[C@H]2OC(=O)NCc2ccc(-c3ccccc3)cc2)cc1]

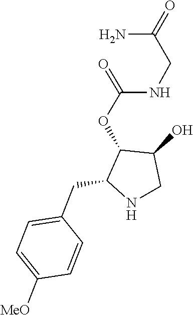 [SMILES: COc1ccc(C[C@H]2NC[C@H](O)[C@H]2OC(=O)NCC(N)=O)cc1]

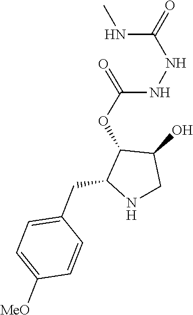 [SMILES: CNC(=O)NNC(=O)O[C@@H]1[C@@H](O)CN[C@@H]1Cc1ccc(OC)cc1]

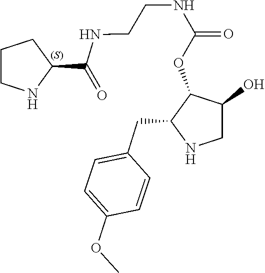

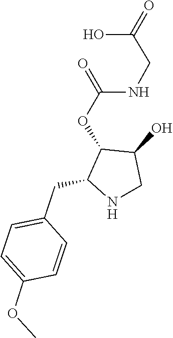 [SMILES: COc1ccc(C[C@H]2NC[C@H](O)[C@H]2OC(=O)NCC(=O)O)cc1]

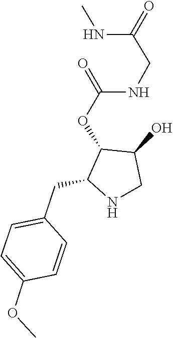 [SMILES: CNC(=O)CNC(=O)O[C@@H]1[C@@H](O)CN[C@@H]1Cc1ccc(OC)cc1]

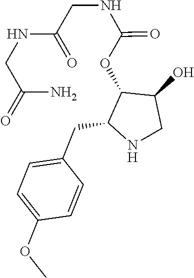 [SMILES: COc1ccc(C[C@H]2NC[C@H](O)[C@H]2OC(=O)NCC(=O)NCC(N)=O)cc1]

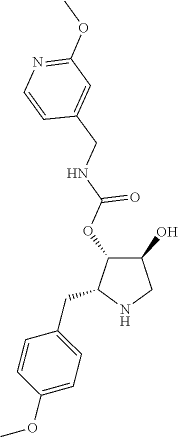 [SMILES: COc1ccc(C[C@H]2NC[C@H](O)[C@H]2OC(=O)NCc2ccnc(OC)c2)cc1]

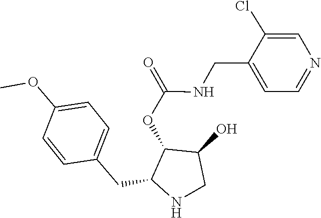 [SMILES: COc1ccc(C[C@H]2NC[C@H](O)[C@H]2OC(=O)NCc2ccncc2Cl)cc1]

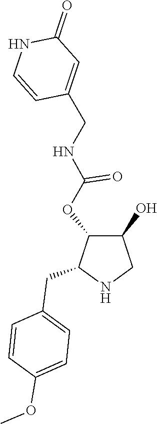 [SMILES: COc1ccc(C[C@H]2NC[C@H](O)[C@H]2OC(=O)NCc2cc[nH]c(=O)c2)cc1]

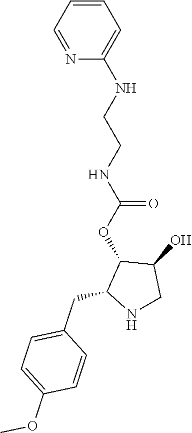 [SMILES: COc1ccc(C[C@H]2NC[C@H](O)[C@H]2OC(=O)NCCNc2ccccn2)cc1]

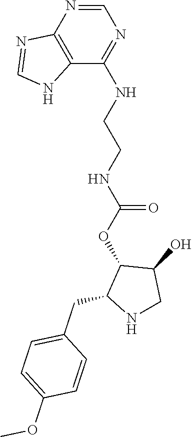 [SMILES: COc1ccc(C[C@H]2NC[C@H](O)[C@H]2OC(=O)NCCNc2ncnc3nc[nH]c23)cc1]

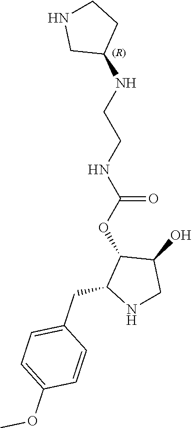

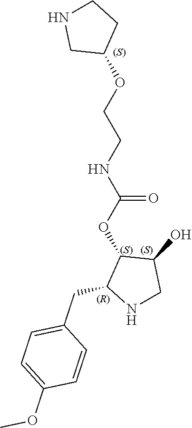

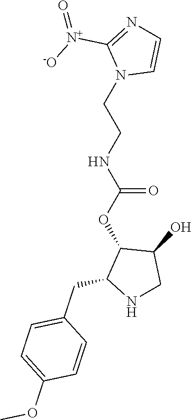

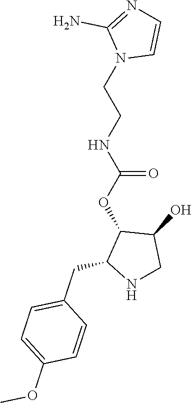 [SMILES: COc1ccc(C[C@H]2NC[C@H](O)[C@H]2OC(=O)NCCn2ccnc2N)cc1]

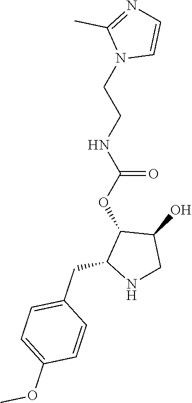 [SMILES: COc1ccc(C[C@H]2NC[C@H](O)[C@H]2OC(=O)NCCn2ccnc2C)cc1]

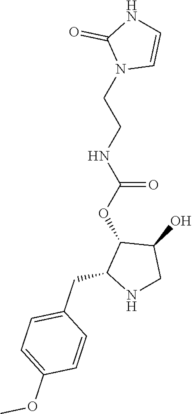 [SMILES: COc1ccc(C[C@H]2NC[C@H](O)[C@H]2OC(=O)NCCn2cc[nH]c2=O)cc1]

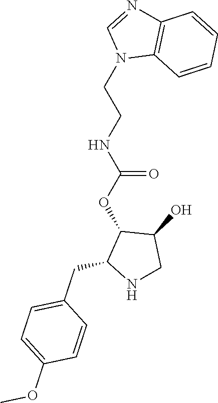 [SMILES: COc1ccc(C[C@H]2NC[C@H](O)[C@H]2OC(=O)NCCn2cnc3ccccc32)cc1]

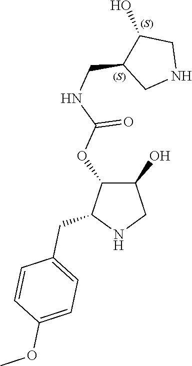

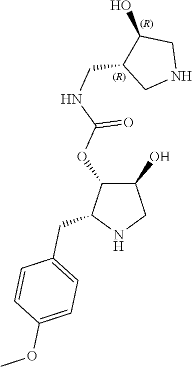

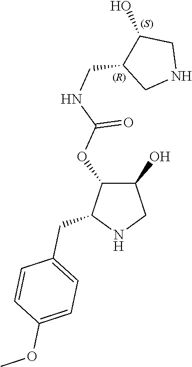

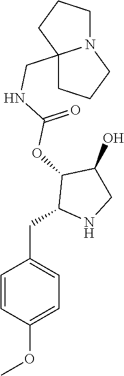 [SMILES: COc1ccc(C[C@H]2NC[C@H](O)[C@H]2OC(=O)NCC23CCCN2CCC3)cc1]

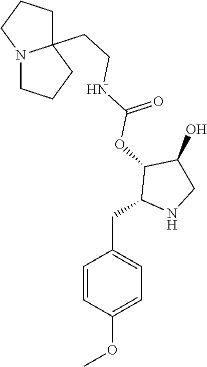 [SMILES: COc1ccc(C[C@H]2NC[C@H](O)[C@H]2OC(=O)NCCC23CCCN2CCC3)cc1]

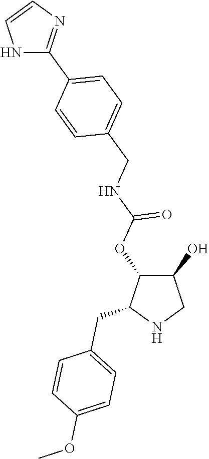 [SMILES: COc1ccc(C[C@H]2NC[C@H](O)[C@H]2OC(=O)NCc2ccc(-c3ncc[nH]3)cc2)cc1]

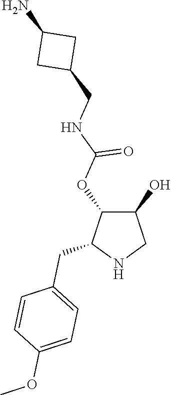 [SMILES: COc1ccc(C[C@H]2NC[C@H](O)[C@H]2OC(=O)NC[C@H]2C[C@@H](N)C2)cc1]

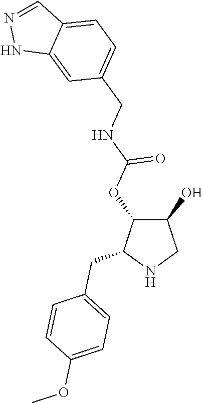 [SMILES: COc1ccc(C[C@H]2NC[C@H](O)[C@H]2OC(=O)NCc2ccc3cn[nH]c3c2)cc1]

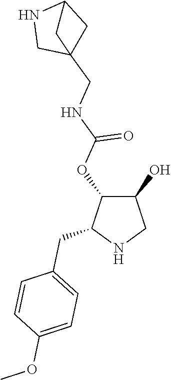 [SMILES: COc1ccc(C[C@H]2NC[C@H](O)[C@H]2OC(=O)NCC23CNC(C2)C3)cc1]

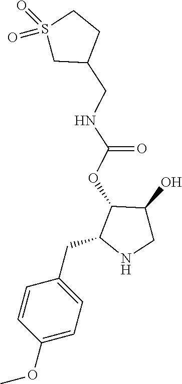 [SMILES: COc1ccc(C[C@H]2NC[C@H](O)[C@H]2OC(=O)NCC2CCS(=O)(=O)C2)cc1]

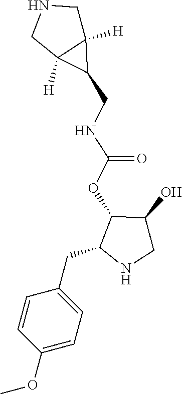 [SMILES: COc1ccc(C[C@H]2NC[C@H](O)[C@H]2OC(=O)NC[C@@H]2[C@@H]3CNC[C@@H]32)cc1]

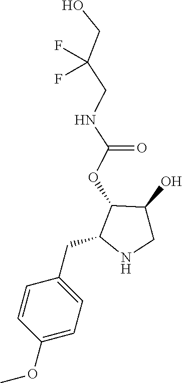 [SMILES: COc1ccc(C[C@H]2NC[C@H](O)[C@H]2OC(=O)NCC(F)(F)CO)cc1]

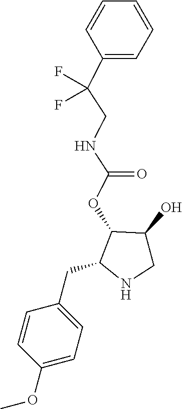 [SMILES: COc1ccc(C[C@H]2NC[C@H](O)[C@H]2OC(=O)NCC(F)(F)c2ccccc2)cc1]

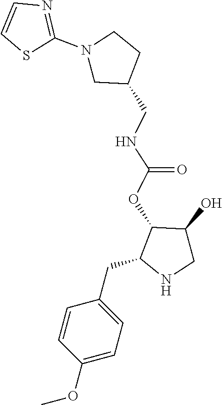 [SMILES: COc1ccc(C[C@H]2NC[C@H](O)[C@H]2OC(=O)NC[C@H]2CCN(c3nccs3)C2)cc1]

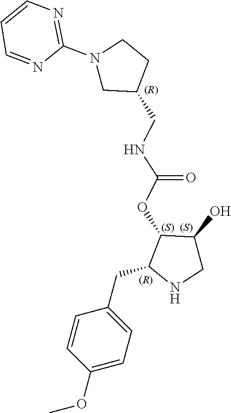

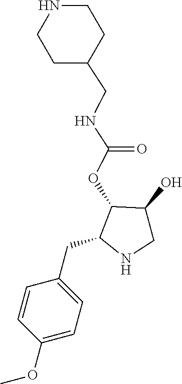 [SMILES: COc1ccc(C[C@H]2NC[C@H](O)[C@H]2OC(=O)NCC2CCNCC2)cc1]

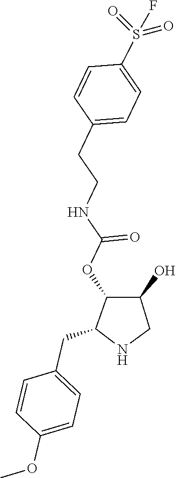 [SMILES: COc1ccc(C[C@H]2NC[C@H](O)[C@H]2OC(=O)NCCc2ccc(S(=O)(=O)F)cc2)cc1]

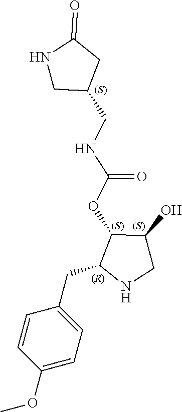

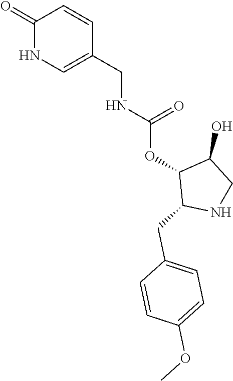 [SMILES: COc1ccc(C[C@H]2NC[C@H](O)[C@H]2OC(=O)NCc2ccc(=O)[nH]c2)cc1]

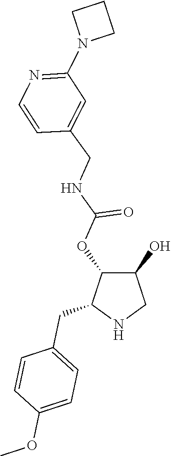 [SMILES: COc1ccc(C[C@H]2NC[C@H](O)[C@H]2OC(=O)NCc2ccnc(N3CCC3)c2)cc1]

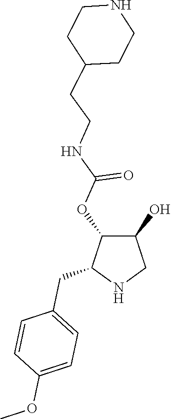 [SMILES: COc1ccc(C[C@H]2NC[C@H](O)[C@H]2OC(=O)NCCC2CCNCC2)cc1]

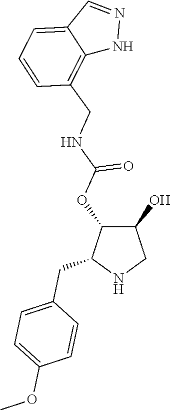 [SMILES: COc1ccc(C[C@H]2NC[C@H](O)[C@H]2OC(=O)NCc2cccc3cn[nH]c23)cc1]

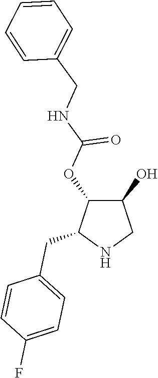 [SMILES: O=C(NCc1ccccc1)O[C@@H]1[C@@H](O)CN[C@@H]1Cc1ccc(F)cc1]

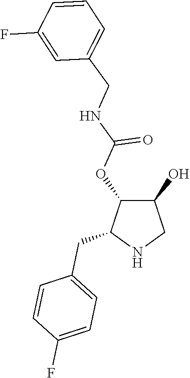 [SMILES: O=C(NCc1cccc(F)c1)O[C@@H]1[C@@H](O)CN[C@@H]1Cc1ccc(F)cc1]

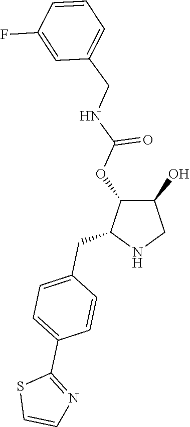 [SMILES: O=C(NCc1cccc(F)c1)O[C@@H]1[C@@H](O)CN[C@@H]1Cc1ccc(-c2nccs2)cc1]

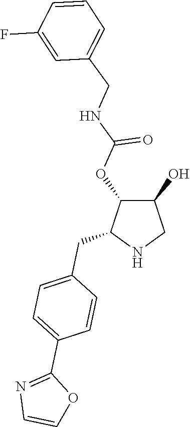 [SMILES: O=C(NCc1cccc(F)c1)O[C@@H]1[C@@H](O)CN[C@@H]1Cc1ccc(-c2ncco2)cc1]

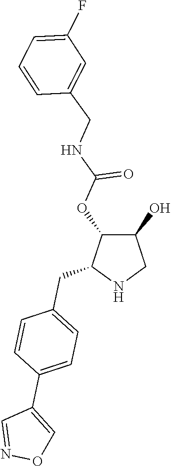 [SMILES: O=C(NCc1cccc(F)c1)O[C@@H]1[C@@H](O)CN[C@@H]1Cc1ccc(-c2cnoc2)cc1]

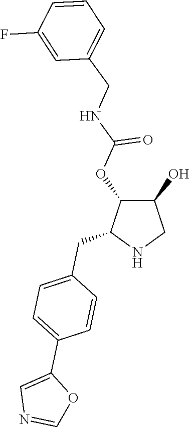 [SMILES: O=C(NCc1cccc(F)c1)O[C@@H]1[C@@H](O)CN[C@@H]1Cc1ccc(-c2cnco2)cc1]

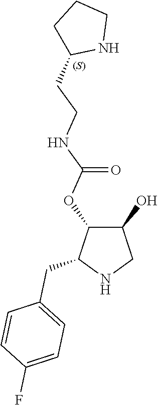

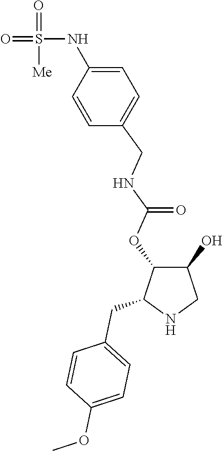 [SMILES: COc1ccc(C[C@H]2NC[C@H](O)[C@H]2OC(=O)NCc2ccc(NS(C)(=O)=O)cc2)cc1]

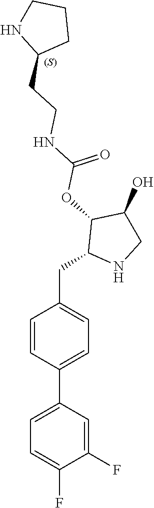

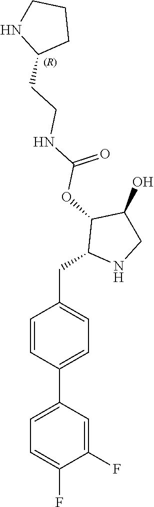

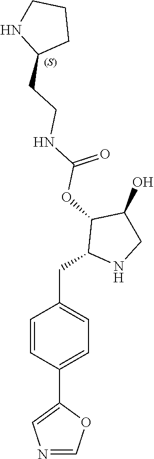

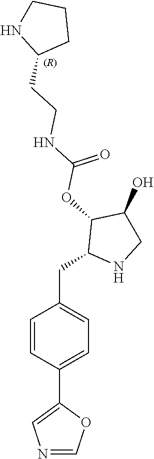

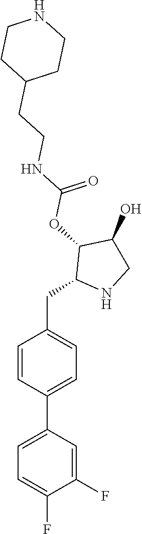 [SMILES: O=C(NCCC1CCNCC1)O[C@@H]1[C@@H](O)CN[C@@H]1Cc1ccc(-c2ccc(F)c(F)c2)cc1]

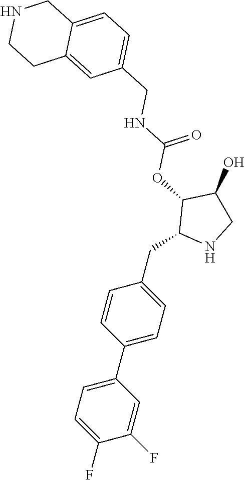 [SMILES: O=C(NCc1ccc2c(c1)CCNC2)O[C@@H]1[C@@H](O)CN[C@@H]1Cc1ccc(-c2ccc(F)c(F)c2)cc1]

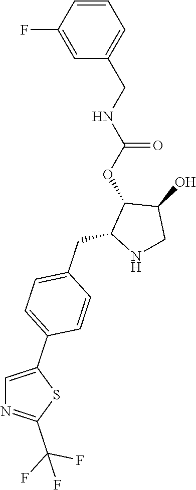 [SMILES: O=C(NCc1cccc(F)c1)O[C@@H]1[C@@H](O)CN[C@@H]1Cc1ccc(-c2cnc(C(F)(F)F)s2)cc1]

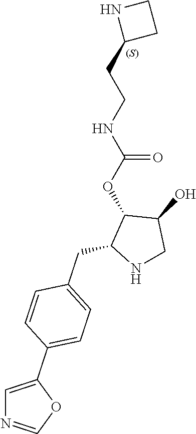

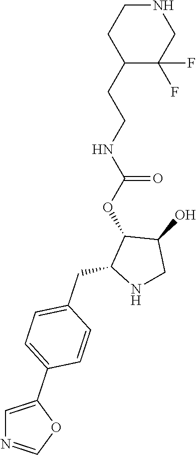 [SMILES: O=C(NCCC1CCNCC1(F)F)O[C@@H]1[C@@H](O)CN[C@@H]1Cc1ccc(-c2cnco2)cc1]

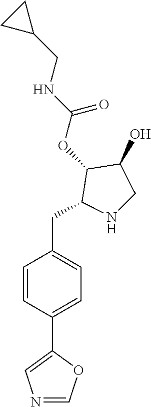 [SMILES: O=C(NCC1CC1)O[C@@H]1[C@@H](O)CN[C@@H]1Cc1ccc(-c2cnco2)cc1]

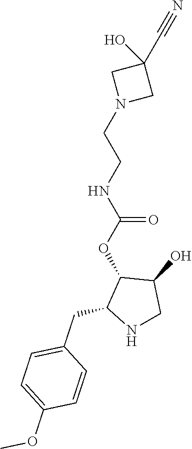 [SMILES: COc1ccc(C[C@H]2NC[C@H](O)[C@H]2OC(=O)NCCN2CC(O)(C#N)C2)cc1]

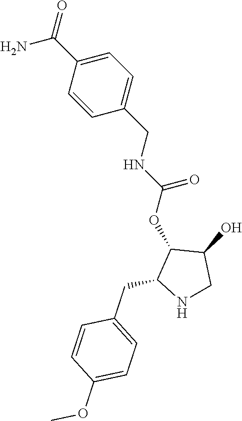 [SMILES: COc1ccc(C[C@H]2NC[C@H](O)[C@H]2OC(=O)NCc2ccc(C(N)=O)cc2)cc1]

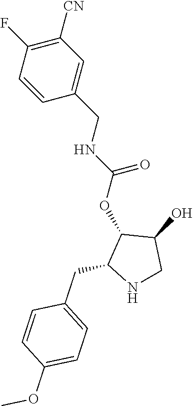 [SMILES: COc1ccc(C[C@H]2NC[C@H](O)[C@H]2OC(=O)NCc2ccc(F)c(C#N)c2)cc1]

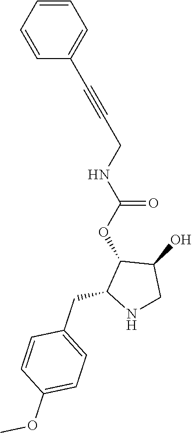 [SMILES: COc1ccc(C[C@H]2NC[C@H](O)[C@H]2OC(=O)NCC#Cc2ccccc2)cc1]

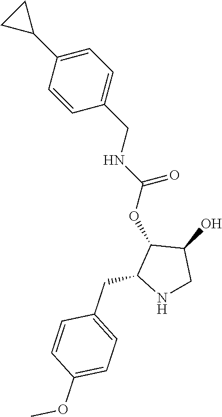 [SMILES: COc1ccc(C[C@H]2NC[C@H](O)[C@H]2OC(=O)NCc2ccc(C3CC3)cc2)cc1]

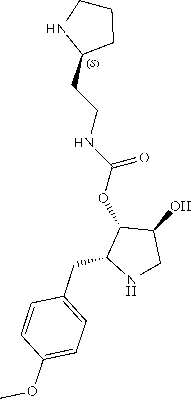

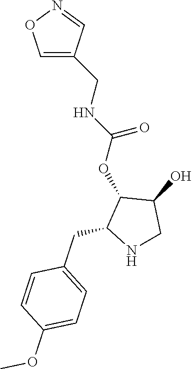 [SMILES: COc1ccc(C[C@H]2NC[C@H](O)[C@H]2OC(=O)NCc2cnoc2)cc1]

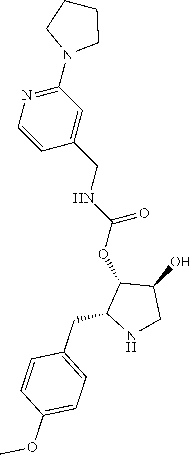 [SMILES: COc1ccc(C[C@H]2NC[C@H](O)[C@H]2OC(=O)NCc2ccnc(N3CCCC3)c2)cc1]

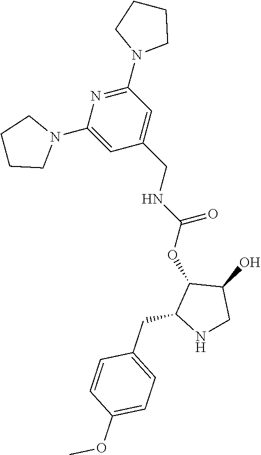 [SMILES: COc1ccc(C[C@H]2NC[C@H](O)[C@H]2OC(=O)NCc2cc(N3CCCC3)nc(N3CCCC3)c2)cc1]

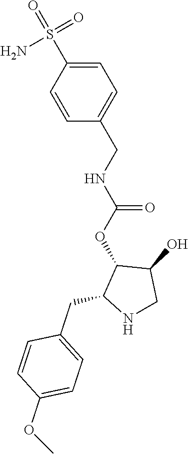 [SMILES: COc1ccc(C[C@H]2NC[C@H](O)[C@H]2OC(=O)NCc2ccc(S(N)(=O)=O)cc2)cc1]

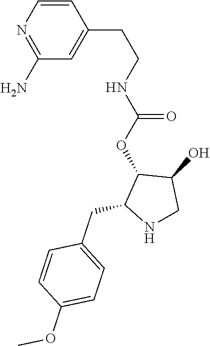 [SMILES: COc1ccc(C[C@H]2NC[C@H](O)[C@H]2OC(=O)NCCc2ccnc(N)c2)cc1]

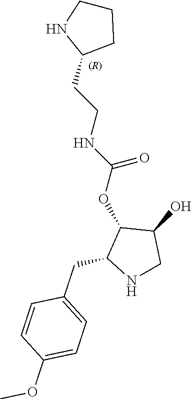

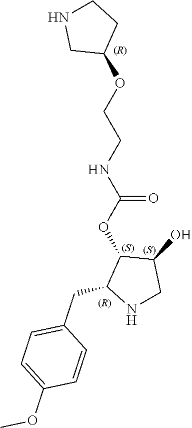

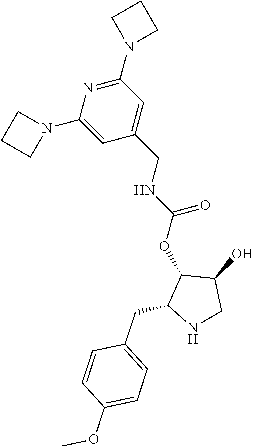 [SMILES: COc1ccc(C[C@H]2NC[C@H](O)[C@H]2OC(=O)NCc2cc(N3CCC3)nc(N3CCC3)c2)cc1]

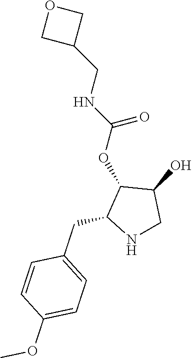 [SMILES: COc1ccc(C[C@H]2NC[C@H](O)[C@H]2OC(=O)NCC2COC2)cc1]

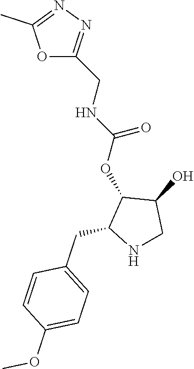 [SMILES: COc1ccc(C[C@H]2NC[C@H](O)[C@H]2OC(=O)NCc2nnc(C)o2)cc1]

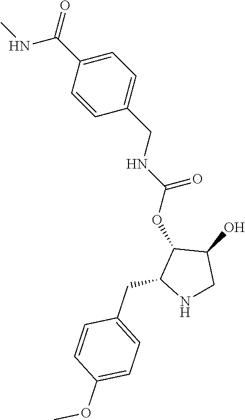 [SMILES: CNC(=O)c1ccc(CNC(=O)O[C@@H]2[C@@H](O)CN[C@@H]2Cc2ccc(OC)cc2)cc1]

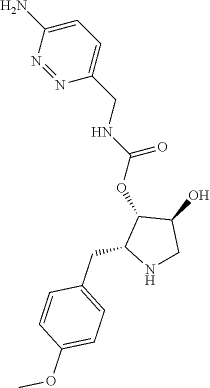 [SMILES: COc1ccc(C[C@H]2NC[C@H](O)[C@H]2OC(=O)NCc2ccc(N)nn2)cc1]

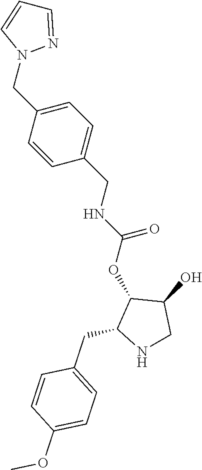 [SMILES: COc1ccc(C[C@H]2NC[C@H](O)[C@H]2OC(=O)NCc2ccc(Cn3cccn3)cc2)cc1]

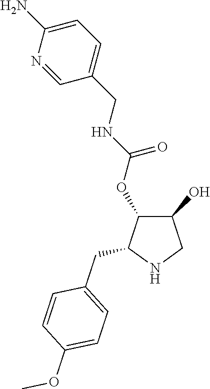 [SMILES: COc1ccc(C[C@H]2NC[C@H](O)[C@H]2OC(=O)NCc2ccc(N)nc2)cc1]

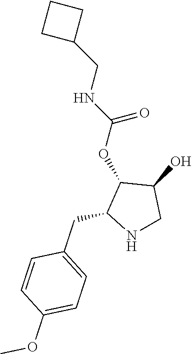 [SMILES: COc1ccc(C[C@H]2NC[C@H](O)[C@H]2OC(=O)NCC2CCC2)cc1]

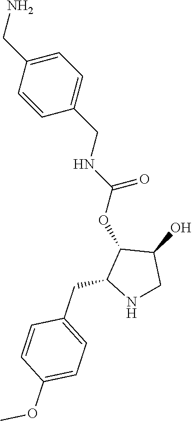 [SMILES: COc1ccc(C[C@H]2NC[C@H](O)[C@H]2OC(=O)NCc2ccc(CN)cc2)cc1]

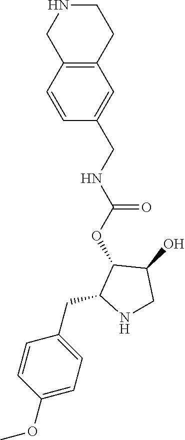 [SMILES: COc1ccc(C[C@H]2NC[C@H](O)[C@H]2OC(=O)NCc2ccc3c(c2)CCNC3)cc1]

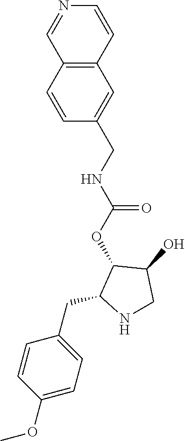 [SMILES: COc1ccc(C[C@H]2NC[C@H](O)[C@H]2OC(=O)NCc2ccc3cnccc3c2)cc1]

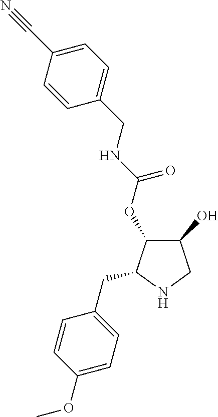 [SMILES: COc1ccc(C[C@H]2NC[C@H](O)[C@H]2OC(=O)NCc2ccc(C#N)cc2)cc1]

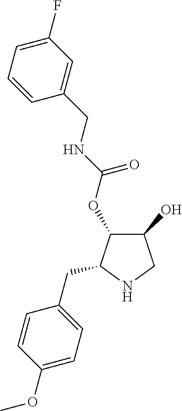 [SMILES: COc1ccc(C[C@H]2NC[C@H](O)[C@H]2OC(=O)NCc2cccc(F)c2)cc1]

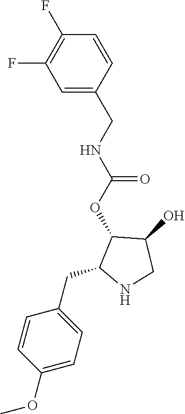 [SMILES: COc1ccc(C[C@H]2NC[C@H](O)[C@H]2OC(=O)NCc2ccc(F)c(F)c2)cc1]

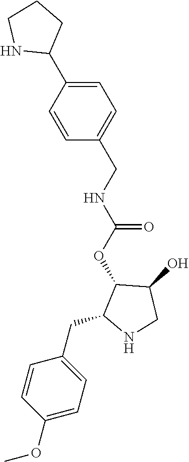 [SMILES: COc1ccc(C[C@H]2NC[C@H](O)[C@H]2OC(=O)NCc2ccc(C3CCCN3)cc2)cc1]

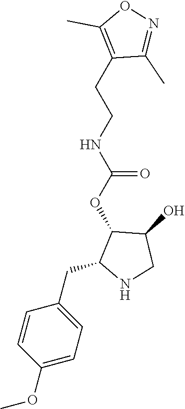 [SMILES: COc1ccc(C[C@H]2NC[C@H](O)[C@H]2OC(=O)NCCc2c(C)noc2C)cc1]

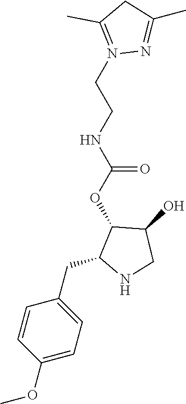

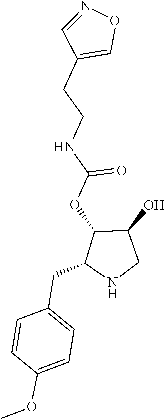 [SMILES: COc1ccc(C[C@H]2NC[C@H](O)[C@H]2OC(=O)NCCc2cnoc2)cc1]

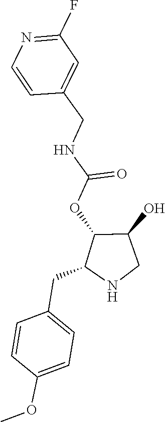 [SMILES: COc1ccc(C[C@H]2NC[C@H](O)[C@H]2OC(=O)NCc2ccnc(F)c2)cc1]

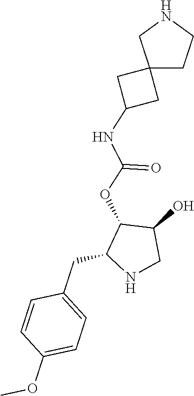 [SMILES: COc1ccc(C[C@H]2NC[C@H](O)[C@H]2OC(=O)NC2CC3(CCNC3)C2)cc1]

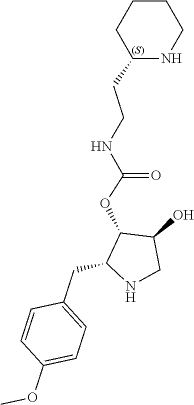

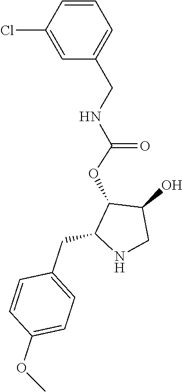 [SMILES: COc1ccc(C[C@H]2NC[C@H](O)[C@H]2OC(=O)NCc2cccc(Cl)c2)cc1]

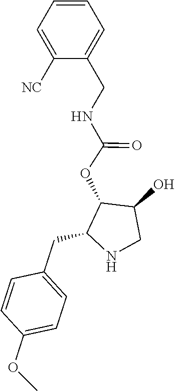 [SMILES: COc1ccc(C[C@H]2NC[C@H](O)[C@H]2OC(=O)NCc2ccccc2C#N)cc1]

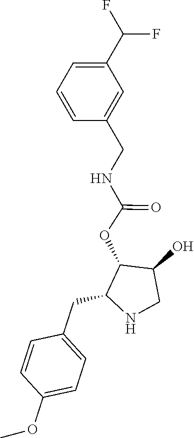 [SMILES: COc1ccc(C[C@H]2NC[C@H](O)[C@H]2OC(=O)NCc2cccc(C(F)F)c2)cc1]

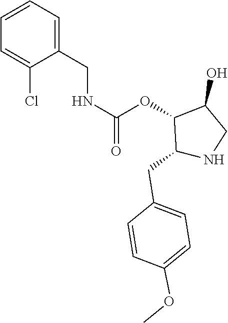 [SMILES: COc1ccc(C[C@H]2NC[C@H](O)[C@H]2OC(=O)NCc2ccccc2Cl)cc1]

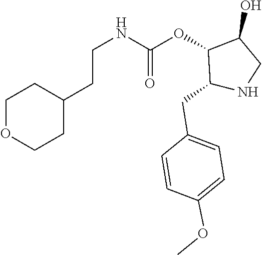 [SMILES: COc1ccc(C[C@H]2NC[C@H](O)[C@H]2OC(=O)NCCC2CCOCC2)cc1]

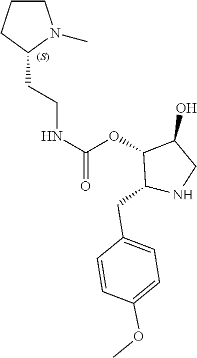

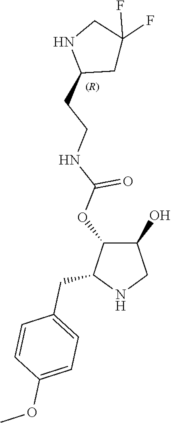

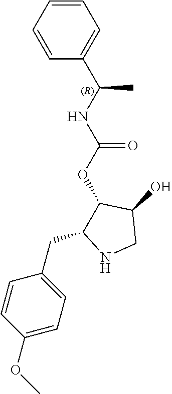

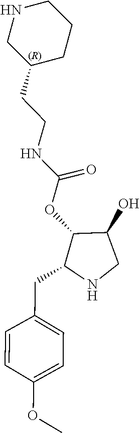

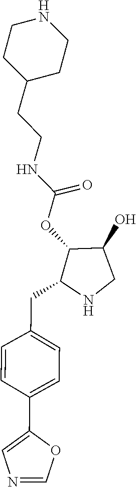 [SMILES: O=C(NCCC1CCNCC1)O[C@@H]1[C@@H](O)CN[C@@H]1Cc1ccc(-c2cnco2)cc1]

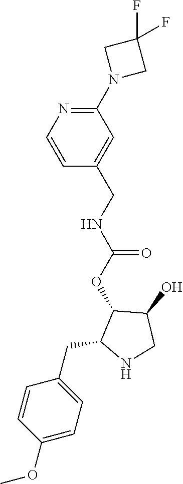 [SMILES: COc1ccc(C[C@H]2NC[C@H](O)[C@H]2OC(=O)NCc2ccnc(N3CC(F)(F)C3)c2)cc1]

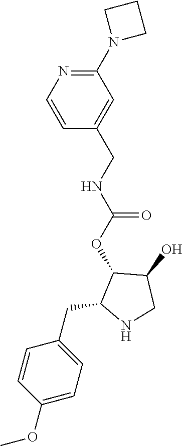 [SMILES: COc1ccc(C[C@H]2NC[C@H](O)[C@H]2OC(=O)NCc2ccnc(N3CCC3)c2)cc1]

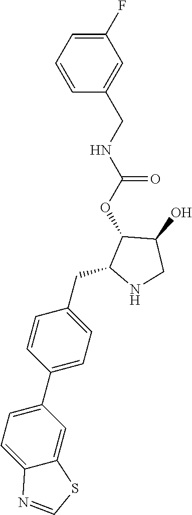 [SMILES: O=C(NCc1cccc(F)c1)O[C@@H]1[C@@H](O)CN[C@@H]1Cc1ccc(-c2ccc3ncsc3c2)cc1]

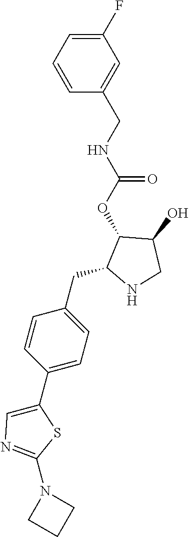 [SMILES: O=C(NCc1cccc(F)c1)O[C@@H]1[C@@H](O)CN[C@@H]1Cc1ccc(-c2cnc(N3CCC3)s2)cc1]

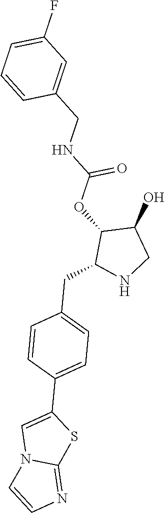 [SMILES: O=C(NCc1cccc(F)c1)O[C@@H]1[C@@H](O)CN[C@@H]1Cc1ccc(-c2cn3ccnc3s2)cc1]

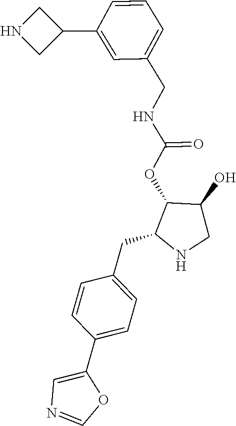 [SMILES: O=C(NCc1cccc(C2CNC2)c1)O[C@@H]1[C@@H](O)CN[C@@H]1Cc1ccc(-c2cnco2)cc1]

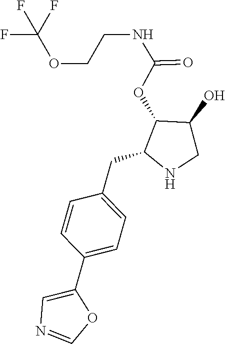 [SMILES: O=C(NCCOC(F)(F)F)O[C@@H]1[C@@H](O)CN[C@@H]1Cc1ccc(-c2cnco2)cc1]

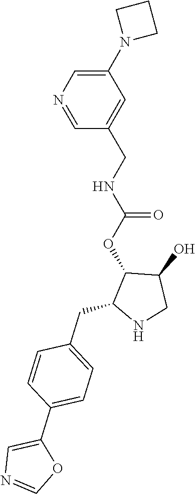 [SMILES: O=C(NCc1cncc(N2CCC2)c1)O[C@@H]1[C@@H](O)CN[C@@H]1Cc1ccc(-c2cnco2)cc1]

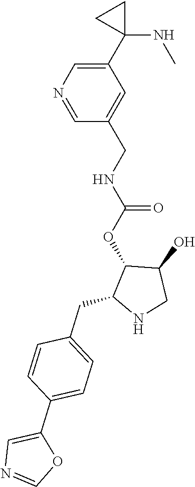 [SMILES: CNC1(c2cncc(CNC(=O)O[C@@H]3[C@@H](O)CN[C@@H]3Cc3ccc(-c4cnco4)cc3)c2)CC1]

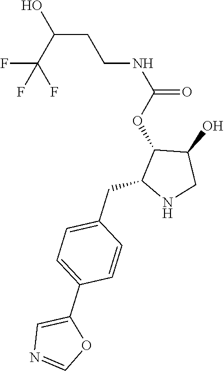 [SMILES: O=C(NCCC(O)C(F)(F)F)O[C@@H]1[C@@H](O)CN[C@@H]1Cc1ccc(-c2cnco2)cc1]

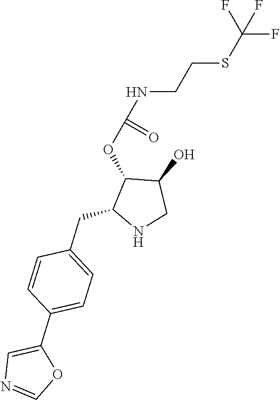 [SMILES: O=C(NCCSC(F)(F)F)O[C@@H]1[C@@H](O)CN[C@@H]1Cc1ccc(-c2cnco2)cc1]

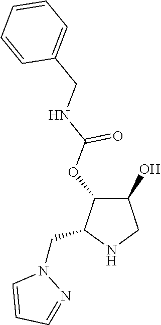 [SMILES: O=C(NCc1ccccc1)O[C@@H]1[C@@H](O)CN[C@@H]1Cn1cccn1]

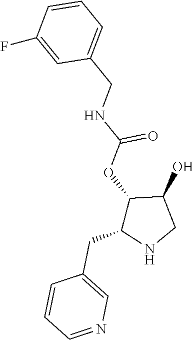 [SMILES: O=C(NCc1cccc(F)c1)O[C@@H]1[C@@H](O)CN[C@@H]1Cc1cccnc1]

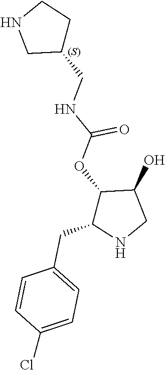

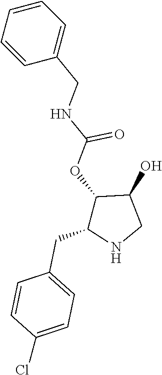 [SMILES: O=C(NCc1ccccc1)O[C@@H]1[C@@H](O)CN[C@@H]1Cc1ccc(Cl)cc1]

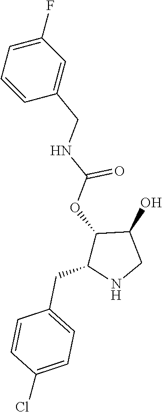 [SMILES: O=C(NCc1cccc(F)c1)O[C@@H]1[C@@H](O)CN[C@@H]1Cc1ccc(Cl)cc1]

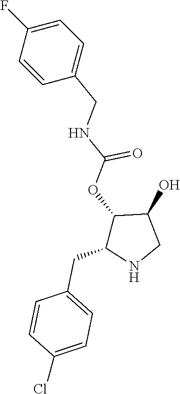 [SMILES: O=C(NCc1ccc(F)cc1)O[C@@H]1[C@@H](O)CN[C@@H]1Cc1ccc(Cl)cc1]

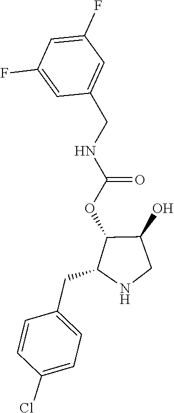 [SMILES: O=C(NCc1cc(F)cc(F)c1)O[C@@H]1[C@@H](O)CN[C@@H]1Cc1ccc(Cl)cc1]

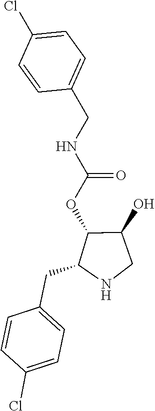 [SMILES: O=C(NCc1ccc(Cl)cc1)O[C@@H]1[C@@H](O)CN[C@@H]1Cc1ccc(Cl)cc1]

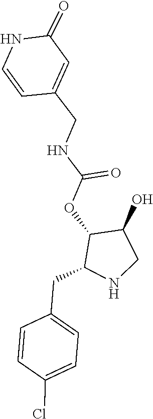 [SMILES: O=C(NCc1cc[nH]c(=O)c1)O[C@@H]1[C@@H](O)CN[C@@H]1Cc1ccc(Cl)cc1]

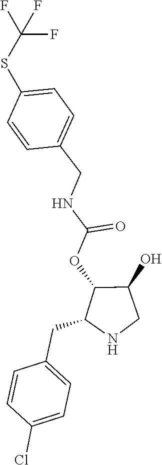 [SMILES: O=C(NCc1ccc(SC(F)(F)F)cc1)O[C@@H]1[C@@H](O)CN[C@@H]1Cc1ccc(Cl)cc1]

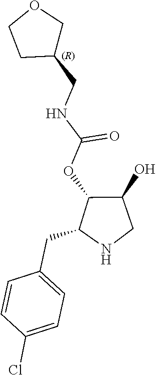

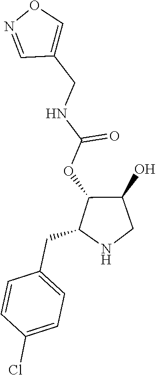 [SMILES: O=C(NCc1cnoc1)O[C@@H]1[C@@H](O)CN[C@@H]1Cc1ccc(Cl)cc1]

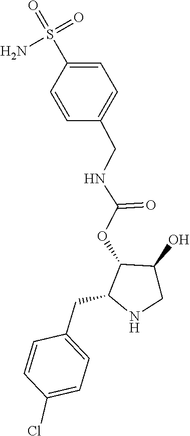 [SMILES: NS(=O)(=O)c1ccc(CNC(=O)O[C@@H]2[C@@H](O)CN[C@@H]2Cc2ccc(Cl)cc2)cc1]

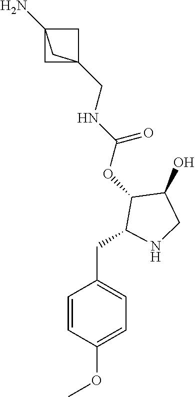 [SMILES: COc1ccc(C[C@H]2NC[C@H](O)[C@H]2OC(=O)NCC23CC(N)(C2)C3)cc1]

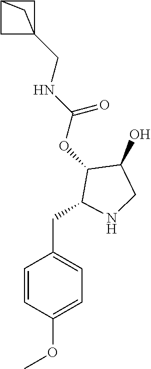 [SMILES: COc1ccc(C[C@H]2NC[C@H](O)[C@H]2OC(=O)NCC23CC(C2)C3)cc1]

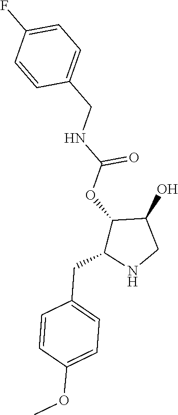 [SMILES: COc1ccc(C[C@H]2NC[C@H](O)[C@H]2OC(=O)NCc2ccc(F)cc2)cc1]

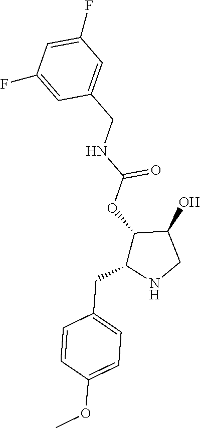 [SMILES: COc1ccc(C[C@H]2NC[C@H](O)[C@H]2OC(=O)NCc2cc(F)cc(F)c2)cc1]

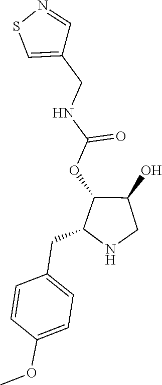 [SMILES: COc1ccc(C[C@H]2NC[C@H](O)[C@H]2OC(=O)NCc2cnsc2)cc1]

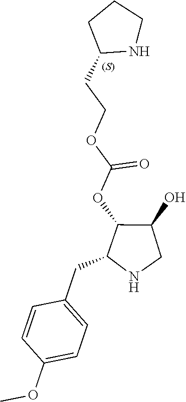

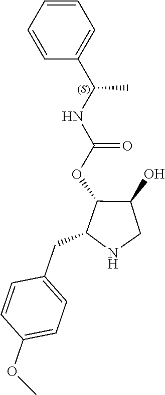

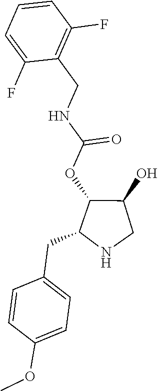 [SMILES: COc1ccc(C[C@H]2NC[C@H](O)[C@H]2OC(=O)NCc2c(F)cccc2F)cc1]

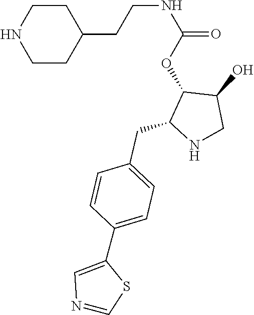 [SMILES: O=C(NCCC1CCNCC1)O[C@@H]1[C@@H](O)CN[C@@H]1Cc1ccc(-c2cncs2)cc1]

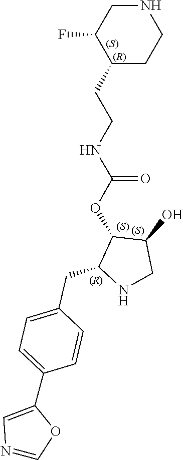

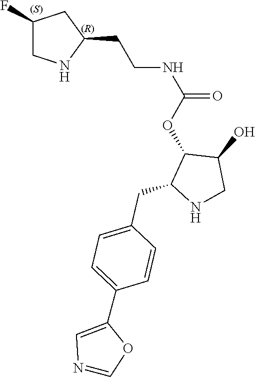

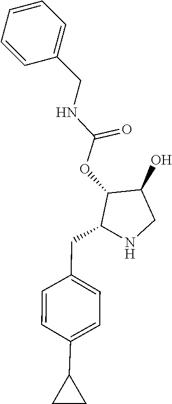 [SMILES: O=C(NCc1ccccc1)O[C@@H]1[C@@H](O)CN[C@@H]1Cc1ccc(C2CC2)cc1]

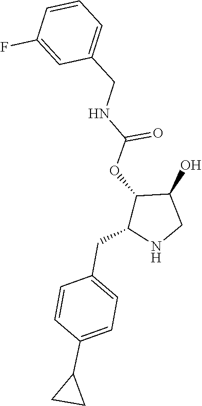 [SMILES: O=C(NCc1cccc(F)c1)O[C@@H]1[C@@H](O)CN[C@@H]1Cc1ccc(C2CC2)cc1]

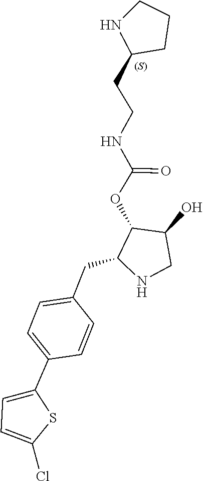

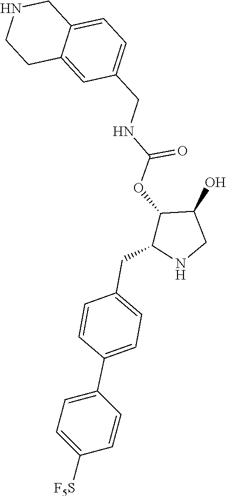

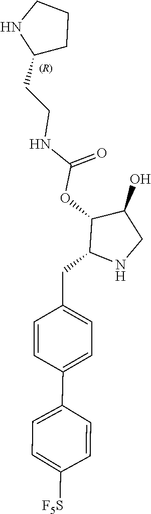

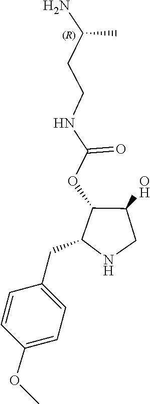

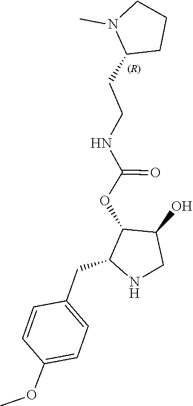

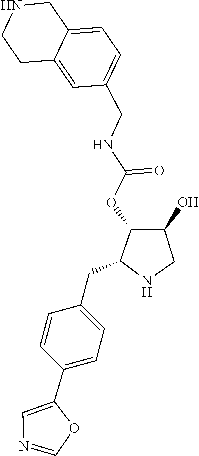 [SMILES: O=C(NCc1ccc2c(c1)CCNC2)O[C@@H]1[C@@H](O)CN[C@@H]1Cc1ccc(-c2cnco2)cc1]

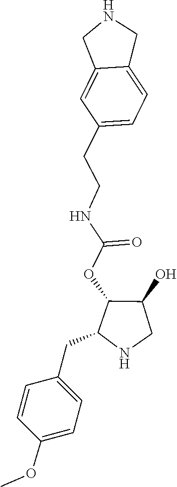 [SMILES: COc1ccc(C[C@H]2NC[C@H](O)[C@H]2OC(=O)NCCc2ccc3c(c2)CNC3)cc1]

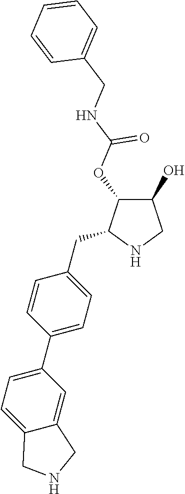 [SMILES: O=C(NCc1ccccc1)O[C@@H]1[C@@H](O)CN[C@@H]1Cc1ccc(-c2ccc3c(c2)CNC3)cc1]

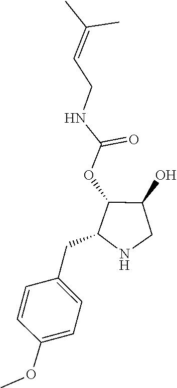 [SMILES: COc1ccc(C[C@H]2NC[C@H](O)[C@H]2OC(=O)NCC=C(C)C)cc1]

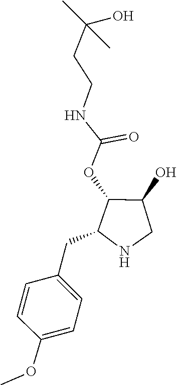 [SMILES: COc1ccc(C[C@H]2NC[C@H](O)[C@H]2OC(=O)NCCC(C)(C)O)cc1]

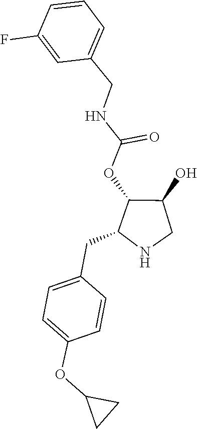 [SMILES: O=C(NCc1cccc(F)c1)O[C@@H]1[C@@H](O)CN[C@@H]1Cc1ccc(OC2CC2)cc1]

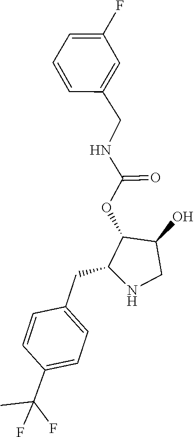 [SMILES: CC(F)(F)c1ccc(C[C@H]2NC[C@H](O)[C@H]2OC(=O)NCc2cccc(F)c2)cc1]

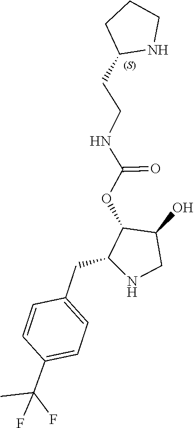

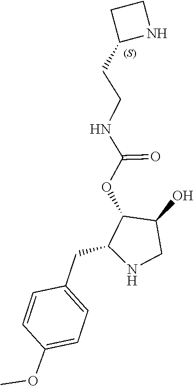

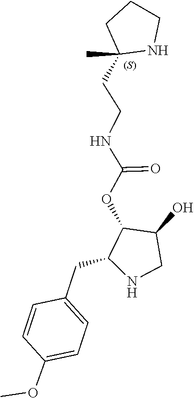

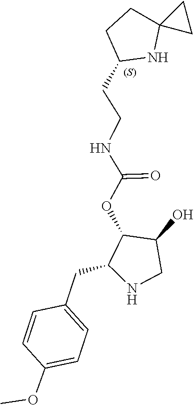

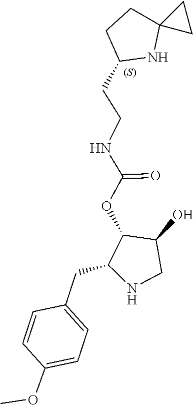

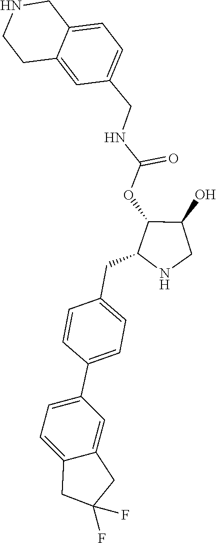 [SMILES: O=C(NCc1ccc2c(c1)CCNC2)O[C@@H]1[C@@H](O)CN[C@@H]1Cc1ccc(-c2ccc3c(c2)CC(F)(F)C3)cc1]

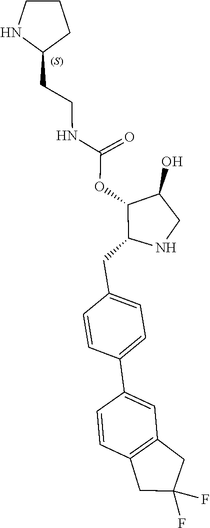

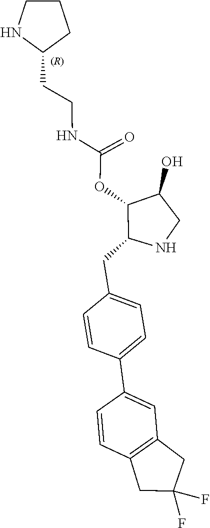

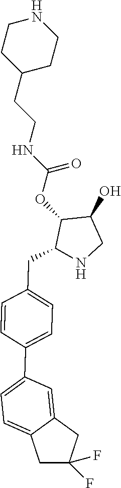 [SMILES: O=C(NCCC1CCNCC1)O[C@@H]1[C@@H](O)CN[C@@H]1Cc1ccc(-c2ccc3c(c2)CC(F)(F)C3)cc1]

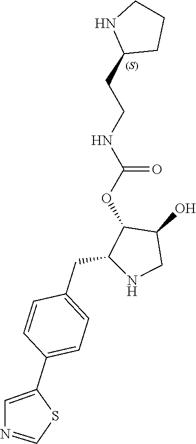

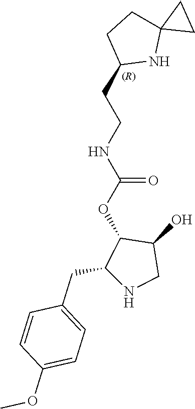

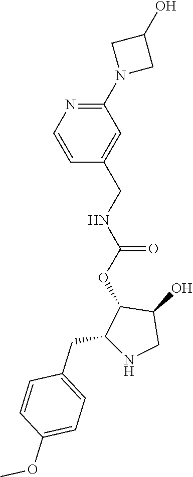 [SMILES: COc1ccc(C[C@H]2NC[C@H](O)[C@H]2OC(=O)NCc2ccnc(N3CC(O)C3)c2)cc1]

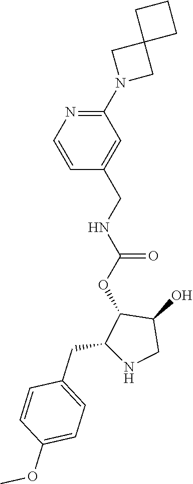 [SMILES: COc1ccc(C[C@H]2NC[C@H](O)[C@H]2OC(=O)NCc2ccnc(N3CC4(CCC4)C3)c2)cc1]

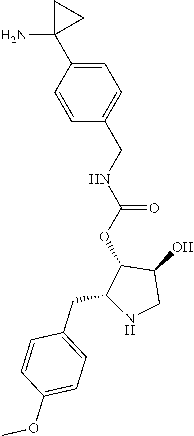 [SMILES: COc1ccc(C[C@H]2NC[C@H](O)[C@H]2OC(=O)NCc2ccc(C3(N)CC3)cc2)cc1]

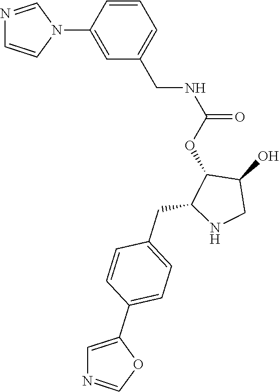 [SMILES: O=C(NCc1cccc(-n2ccnc2)c1)O[C@@H]1[C@@H](O)CN[C@@H]1Cc1ccc(-c2cnco2)cc1]

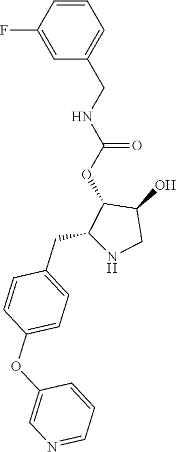 [SMILES: O=C(NCc1cccc(F)c1)O[C@@H]1[C@@H](O)CN[C@@H]1Cc1ccc(Oc2cccnc2)cc1]

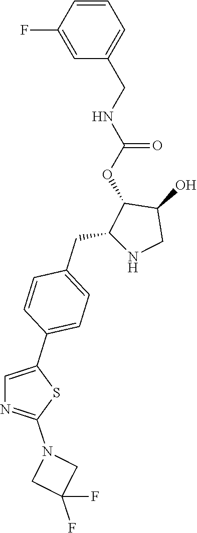 [SMILES: O=C(NCc1cccc(F)c1)O[C@@H]1[C@@H](O)CN[C@@H]1Cc1ccc(-c2cnc(N3CC(F)(F)C3)s2)cc1]

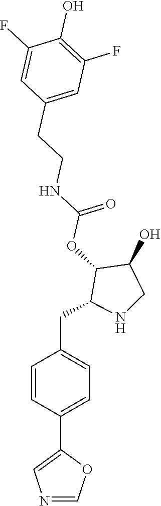 [SMILES: O=C(NCCc1cc(F)c(O)c(F)c1)O[C@@H]1[C@@H](O)CN[C@@H]1Cc1ccc(-c2cnco2)cc1]

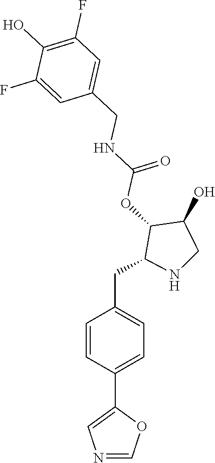 [SMILES: O=C(NCc1cc(F)c(O)c(F)c1)O[C@@H]1[C@@H](O)CN[C@@H]1Cc1ccc(-c2cnco2)cc1]

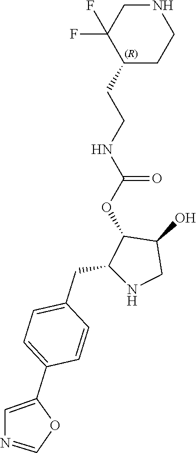

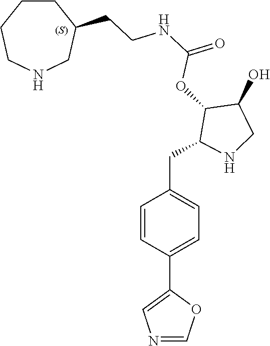

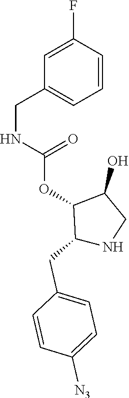 [SMILES: [N-]=[N+]=Nc1ccc(C[C@H]2NC[C@H](O)[C@H]2OC(=O)NCc2cccc(F)c2)cc1]

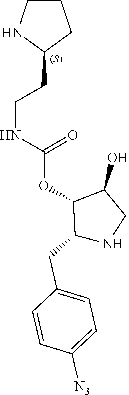

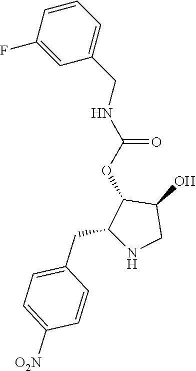 [SMILES: O=C(NCc1cccc(F)c1)O[C@@H]1[C@@H](O)CN[C@@H]1Cc1ccc([N+](=O)[O-])cc1]

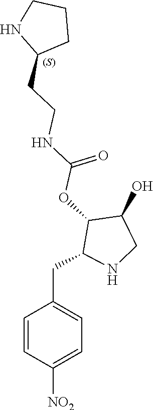

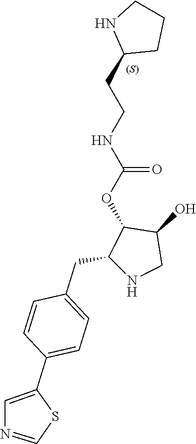

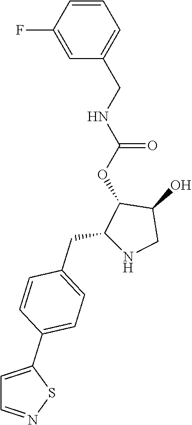 [SMILES: O=C(NCc1cccc(F)c1)O[C@@H]1[C@@H](O)CN[C@@H]1Cc1ccc(-c2ccns2)cc1]

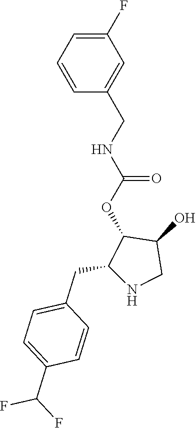 [SMILES: O=C(NCc1cccc(F)c1)O[C@@H]1[C@@H](O)CN[C@@H]1Cc1ccc(C(F)F)cc1]

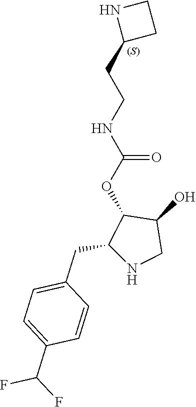

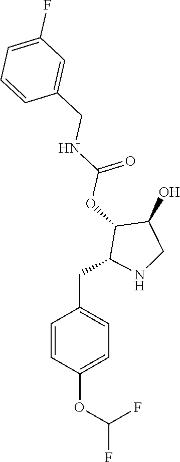 [SMILES: O=C(NCc1cccc(F)c1)O[C@@H]1[C@@H](O)CN[C@@H]1Cc1ccc(OC(F)F)cc1]

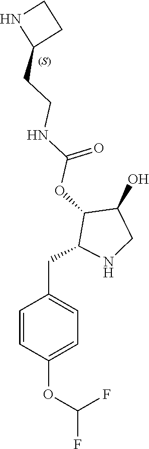

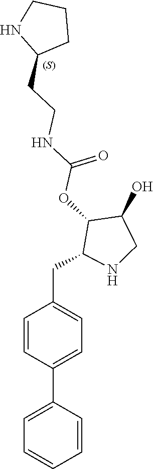

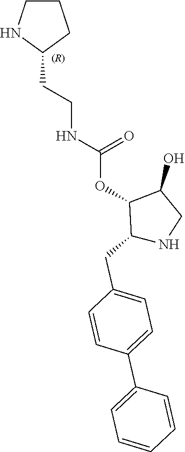

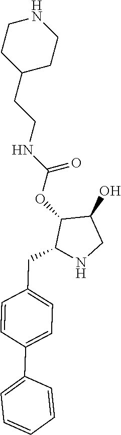 [SMILES: O=C(NCCC1CCNCC1)O[C@@H]1[C@@H](O)CN[C@@H]1Cc1ccc(-c2ccccc2)cc1]

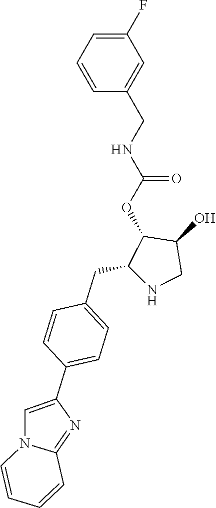 [SMILES: O=C(NCc1cccc(F)c1)O[C@@H]1[C@@H](O)CN[C@@H]1Cc1ccc(-c2cn3ccccc3n2)cc1]

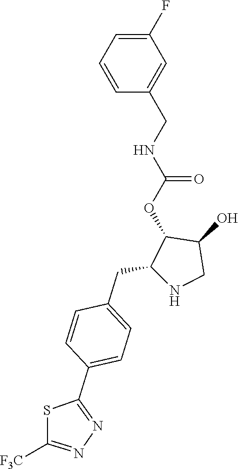 [SMILES: O=C(NCc1cccc(F)c1)O[C@@H]1[C@@H](O)CN[C@@H]1Cc1ccc(-c2nnc(C(F)(F)F)s2)cc1]

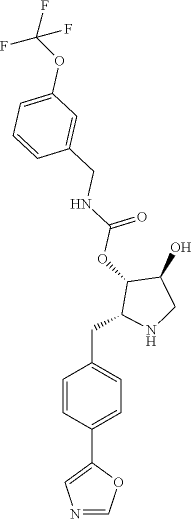 [SMILES: O=C(NCc1cccc(OC(F)(F)F)c1)O[C@@H]1[C@@H](O)CN[C@@H]1Cc1ccc(-c2cnco2)cc1]

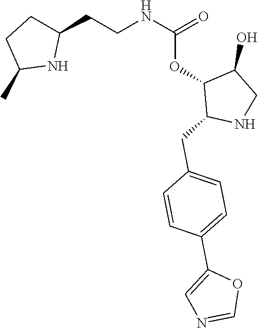 [SMILES: C[C@H]1CC[C@@H](CCNC(=O)O[C@@H]2[C@@H](O)CN[C@@H]2Cc2ccc(-c3cnco3)cc2)N1]

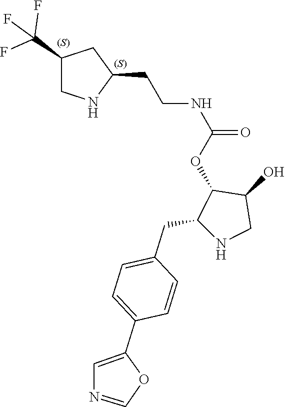

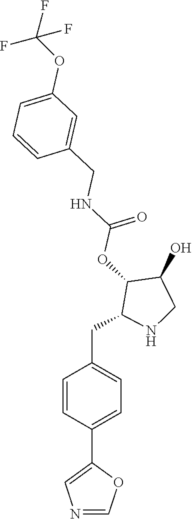 [SMILES: O=C(NCc1cccc(OC(F)(F)F)c1)O[C@@H]1[C@@H](O)CN[C@@H]1Cc1ccc(-c2cnco2)cc1]

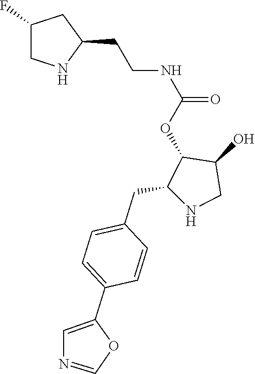 [SMILES: O=C(NCC[C@@H]1C[C@@H](F)CN1)O[C@@H]1[C@@H](O)CN[C@@H]1Cc1ccc(-c2cnco2)cc1]

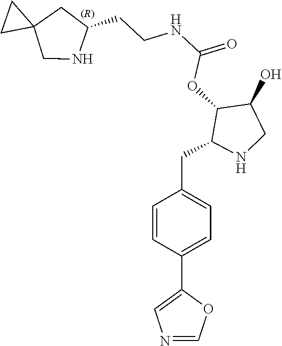

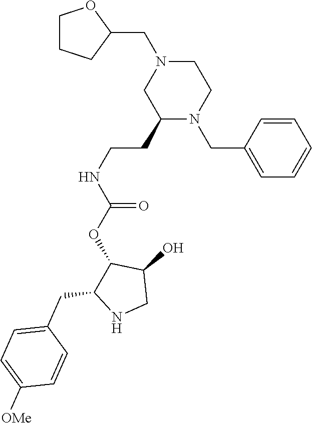 [SMILES: COc1ccc(C[C@H]2NC[C@H](O)[C@H]2OC(=O)NCC[C@H]2CN(CC3CCCO3)CCN2Cc2ccccc2)cc1]

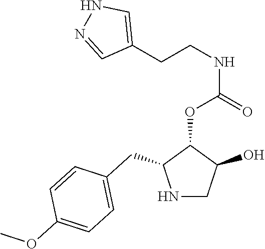 [SMILES: COc1ccc(C[C@H]2NC[C@H](O)[C@H]2OC(=O)NCCc2cn[nH]c2)cc1]

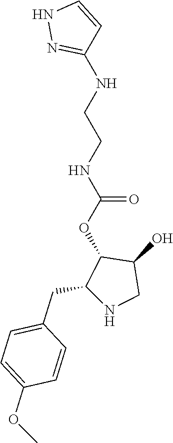 [SMILES: COc1ccc(C[C@H]2NC[C@H](O)[C@H]2OC(=O)NCCNc2cc[nH]n2)cc1]

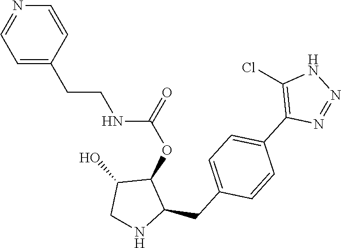 [SMILES: O=C(NCCc1ccncc1)O[C@@H]1[C@@H](O)CN[C@@H]1Cc1ccc(-c2nn[nH]c2Cl)cc1]

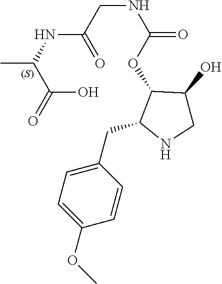

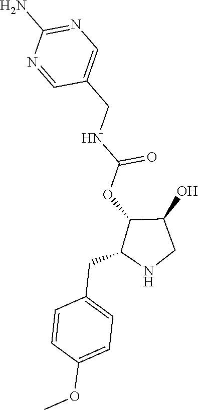 [SMILES: COc1ccc(C[C@H]2NC[C@H](O)[C@H]2OC(=O)NCc2cnc(N)nc2)cc1]

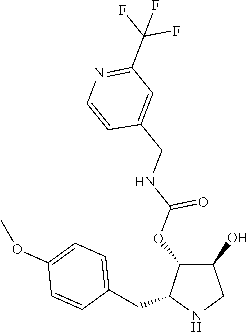 [SMILES: COc1ccc(C[C@H]2NC[C@H](O)[C@H]2OC(=O)NCc2ccnc(C(F)(F)F)c2)cc1]

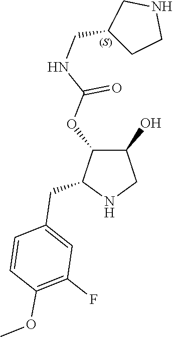

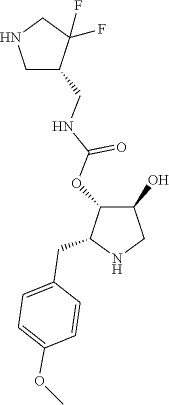 [SMILES: COc1ccc(C[C@H]2NC[C@H](O)[C@H]2OC(=O)NC[C@@H]2CNCC2(F)F)cc1]

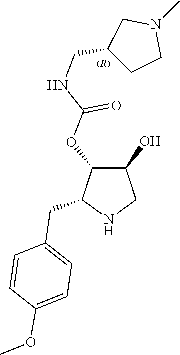

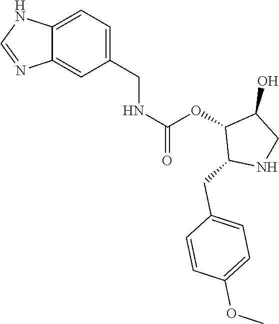 [SMILES: COc1ccc(C[C@H]2NC[C@H](O)[C@H]2OC(=O)NCc2ccc3[nH]cnc3c2)cc1]

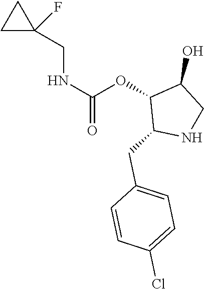 [SMILES: O=C(NCC1(F)CC1)O[C@@H]1[C@@H](O)CN[C@@H]1Cc1ccc(Cl)cc1]

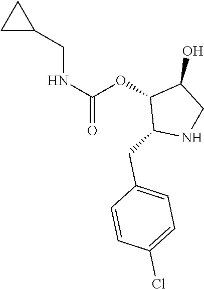 [SMILES: O=C(NCC1CC1)O[C@@H]1[C@@H](O)CN[C@@H]1Cc1ccc(Cl)cc1]

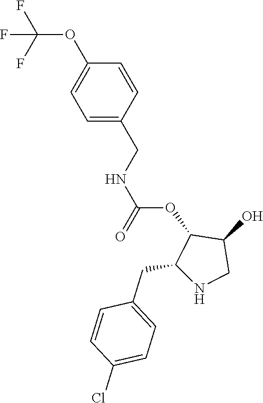 [SMILES: O=C(NCc1ccc(OC(F)(F)F)cc1)O[C@@H]1[C@@H](O)CN[C@@H]1Cc1ccc(Cl)cc1]

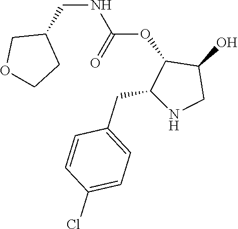 [SMILES: O=C(NC[C@@H]1CCOC1)O[C@@H]1[C@@H](O)CN[C@@H]1Cc1ccc(Cl)cc1]

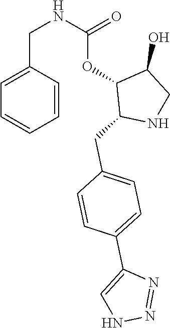 [SMILES: O=C(NCc1ccccc1)O[C@@H]1[C@@H](O)CN[C@@H]1Cc1ccc(-c2c[nH]nn2)cc1]

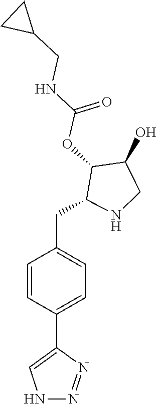 [SMILES: O=C(NCC1CC1)O[C@@H]1[C@@H](O)CN[C@@H]1Cc1ccc(-c2c[nH]nn2)cc1]

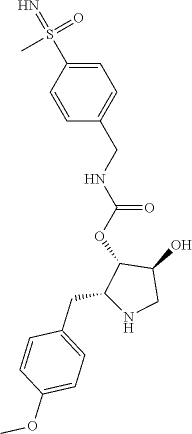 [SMILES: COc1ccc(C[C@H]2NC[C@H](O)[C@H]2OC(=O)NCc2ccc(S(C)(=N)=O)cc2)cc1]

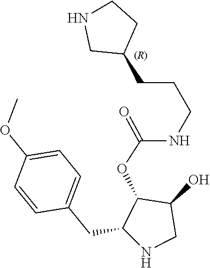

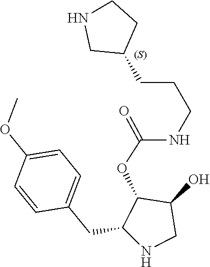

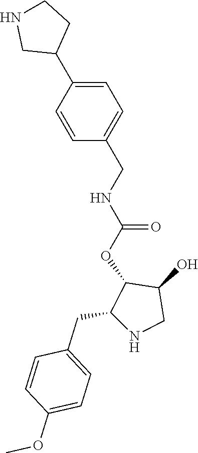 [SMILES: COc1ccc(C[C@H]2NC[C@H](O)[C@H]2OC(=O)NCc2ccc(C3CCNC3)cc2)cc1]

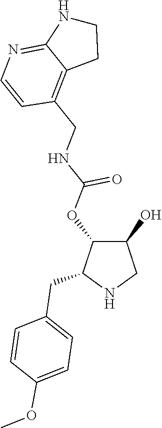 [SMILES: COc1ccc(C[C@H]2NC[C@H](O)[C@H]2OC(=O)NCc2ccnc3c2CCN3)cc1]

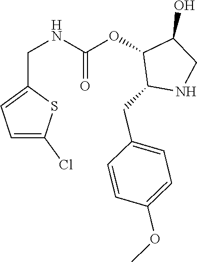 [SMILES: COc1ccc(C[C@H]2NC[C@H](O)[C@H]2OC(=O)NCc2ccc(Cl)s2)cc1]

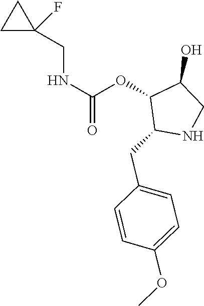 [SMILES: COc1ccc(C[C@H]2NC[C@H](O)[C@H]2OC(=O)NCC2(F)CC2)cc1]

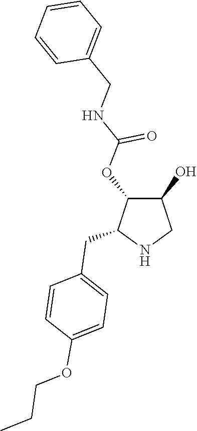 [SMILES: CCCOc1ccc(C[C@H]2NC[C@H](O)[C@H]2OC(=O)NCc2ccccc2)cc1]

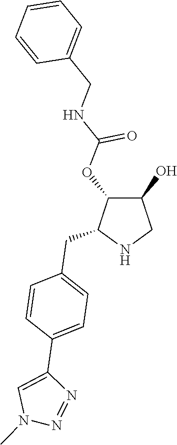 [SMILES: Cn1cc(-c2ccc(C[C@H]3NC[C@H](O)[C@H]3OC(=O)NCc3ccccc3)cc2)nn1]

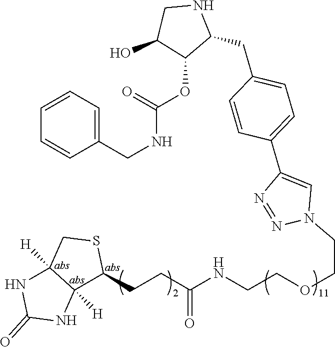

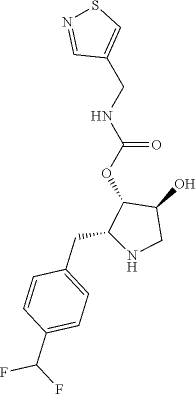 [SMILES: O=C(NCc1cnsc1)O[C@@H]1[C@@H](O)CN[C@@H]1Cc1ccc(C(F)F)cc1]

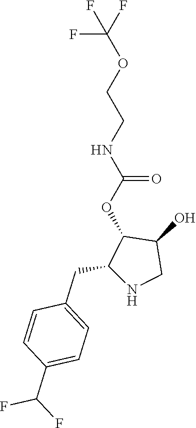 [SMILES: O=C(NCCOC(F)(F)F)O[C@@H]1[C@@H](O)CN[C@@H]1Cc1ccc(C(F)F)cc1]

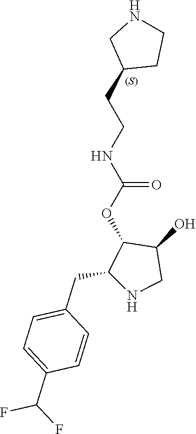

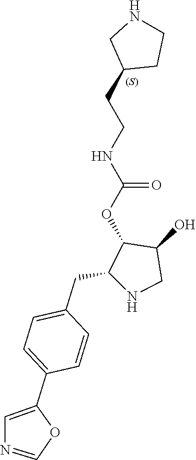

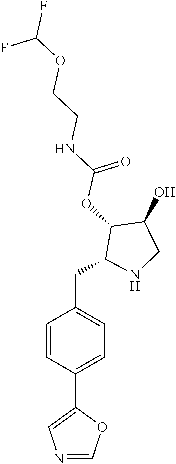 [SMILES: O=C(NCCOC(F)F)O[C@@H]1[C@@H](O)CN[C@@H]1Cc1ccc(-c2cnco2)cc1]

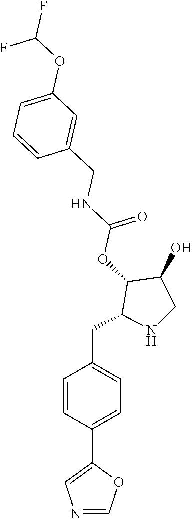 [SMILES: O=C(NCc1cccc(OC(F)F)c1)O[C@@H]1[C@@H](O)CN[C@@H]1Cc1ccc(-c2cnco2)cc1]

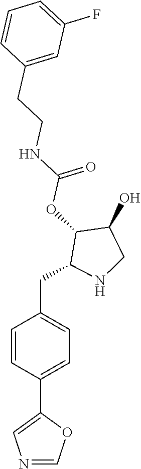 [SMILES: O=C(NCCc1cccc(F)c1)O[C@@H]1[C@@H](O)CN[C@@H]1Cc1ccc(-c2cnco2)cc1]

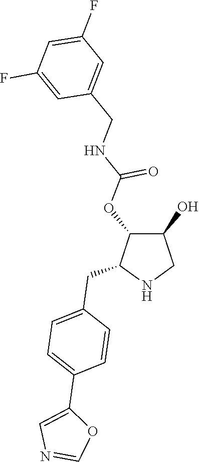 [SMILES: O=C(NCc1cc(F)cc(F)c1)O[C@@H]1[C@@H](O)CN[C@@H]1Cc1ccc(-c2cnco2)cc1]

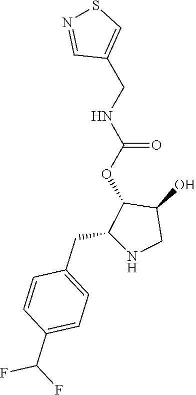 [SMILES: O=C(NCc1cnsc1)O[C@@H]1[C@@H](O)CN[C@@H]1Cc1ccc(C(F)F)cc1]

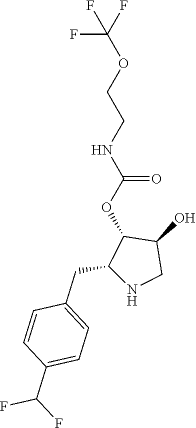 [SMILES: O=C(NCCOC(F)(F)F)O[C@@H]1[C@@H](O)CN[C@@H]1Cc1ccc(C(F)F)cc1]

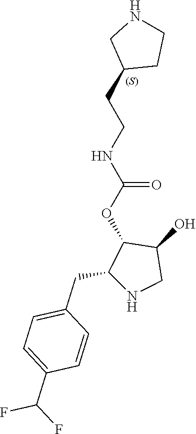

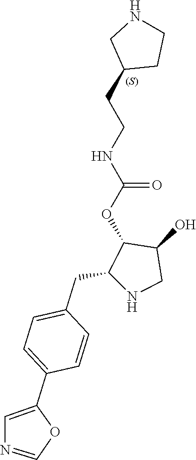

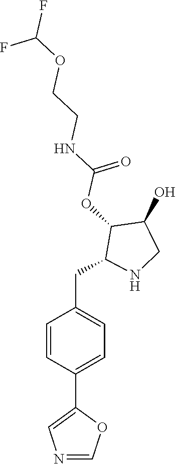 [SMILES: O=C(NCCOC(F)F)O[C@@H]1[C@@H](O)CN[C@@H]1Cc1ccc(-c2cnco2)cc1]

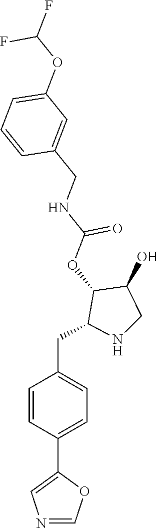 [SMILES: O=C(NCc1cccc(OC(F)F)c1)O[C@@H]1[C@@H](O)CN[C@@H]1Cc1ccc(-c2cnco2)cc1]

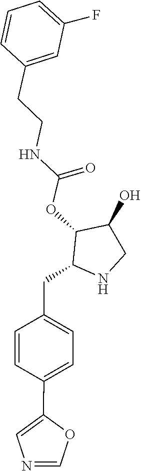 [SMILES: O=C(NCCc1cccc(F)c1)O[C@@H]1[C@@H](O)CN[C@@H]1Cc1ccc(-c2cnco2)cc1]

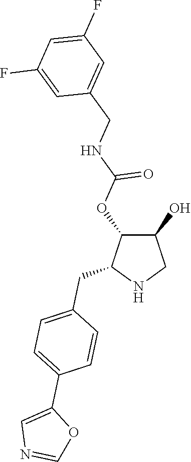 [SMILES: O=C(NCc1cc(F)cc(F)c1)O[C@@H]1[C@@H](O)CN[C@@H]1Cc1ccc(-c2cnco2)cc1]

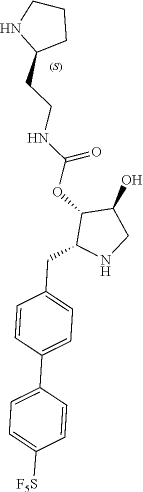

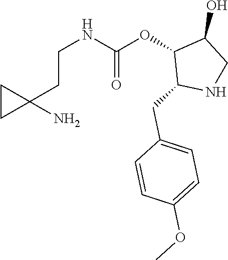 [SMILES: COc1ccc(C[C@H]2NC[C@H](O)[C@H]2OC(=O)NCCC2(N)CC2)cc1]

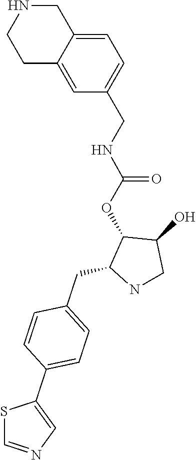

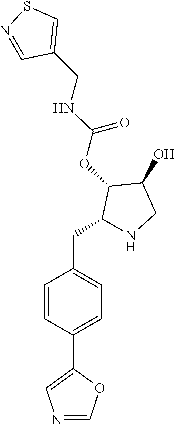 [SMILES: O=C(NCc1cnsc1)O[C@@H]1[C@@H](O)CN[C@@H]1Cc1ccc(-c2cnco2)cc1]

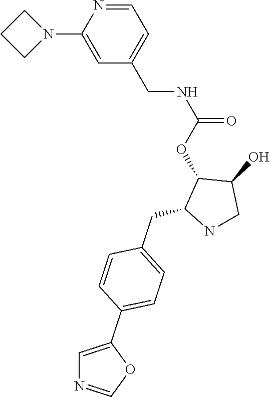

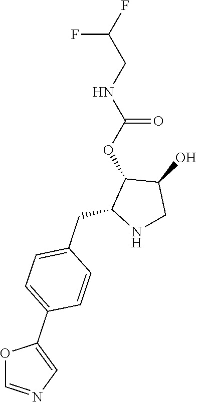 [SMILES: O=C(NCC(F)F)O[C@@H]1[C@@H](O)CN[C@@H]1Cc1ccc(-c2cnco2)cc1]

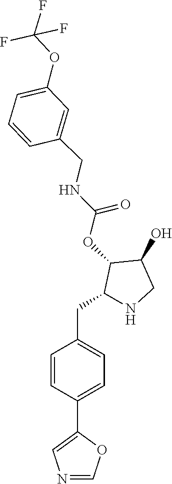 [SMILES: O=C(NCc1cccc(OC(F)(F)F)c1)O[C@@H]1[C@@H](O)CN[C@@H]1Cc1ccc(-c2cnco2)cc1]

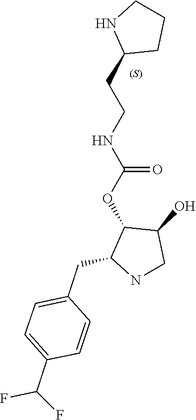

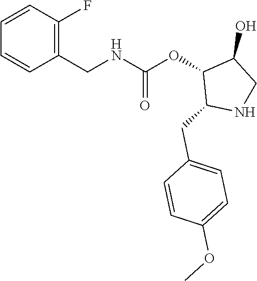 [SMILES: COc1ccc(C[C@H]2NC[C@H](O)[C@H]2OC(=O)NCc2ccccc2F)cc1]

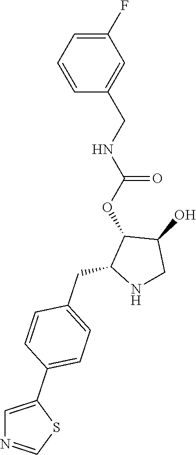 [SMILES: O=C(NCc1cccc(F)c1)O[C@@H]1[C@@H](O)CN[C@@H]1Cc1ccc(-c2cncs2)cc1]

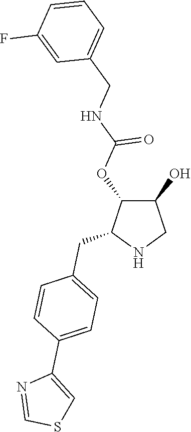 [SMILES: O=C(NCc1cccc(F)c1)O[C@@H]1[C@@H](O)CN[C@@H]1Cc1ccc(-c2cscn2)cc1]

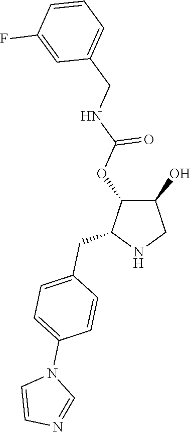 [SMILES: O=C(NCc1cccc(F)c1)O[C@@H]1[C@@H](O)CN[C@@H]1Cc1ccc(-n2ccnc2)cc1]

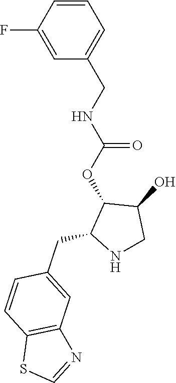 [SMILES: O=C(NCc1cccc(F)c1)O[C@@H]1[C@@H](O)CN[C@@H]1Cc1ccc2scnc2c1]

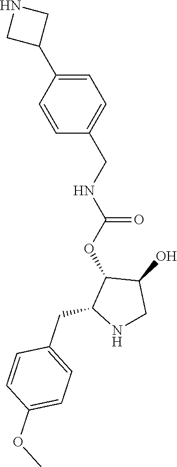 [SMILES: COc1ccc(C[C@H]2NC[C@H](O)[C@H]2OC(=O)NCc2ccc(C3CNC3)cc2)cc1]

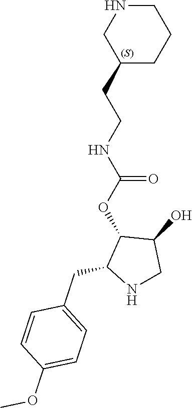

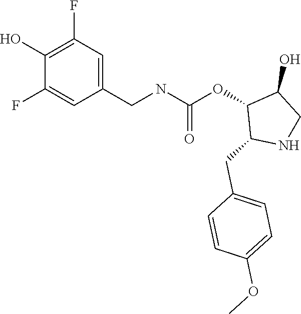

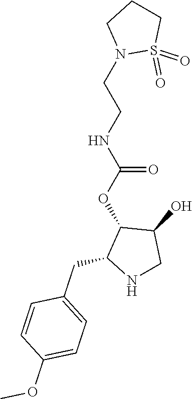 [SMILES: COc1ccc(C[C@H]2NC[C@H](O)[C@H]2OC(=O)NCCN2CCCS2(=O)=O)cc1]

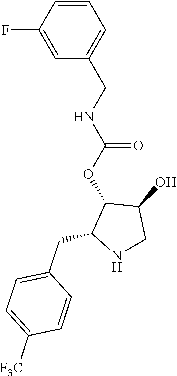 [SMILES: O=C(NCc1cccc(F)c1)O[C@@H]1[C@@H](O)CN[C@@H]1Cc1ccc(C(F)(F)F)cc1]

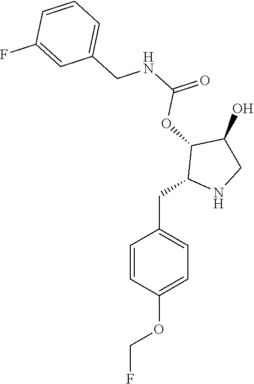 [SMILES: O=C(NCc1cccc(F)c1)O[C@@H]1[C@@H](O)CN[C@@H]1Cc1ccc(OCF)cc1]

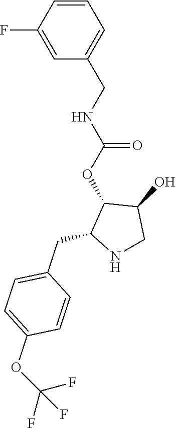 [SMILES: O=C(NCc1cccc(F)c1)O[C@@H]1[C@@H](O)CN[C@@H]1Cc1ccc(OC(F)(F)F)cc1]

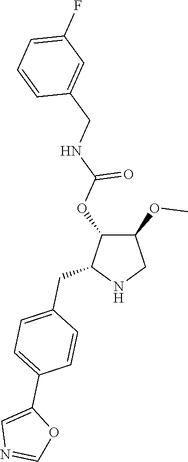 [SMILES: CO[C@H]1CN[C@H](Cc2ccc(-c3cnco3)cc2)[C@@H]1OC(=O)NCc1cccc(F)c1]

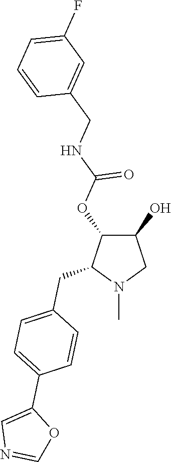 [SMILES: CN1C[C@H](O)[C@@H](OC(=O)NCc2cccc(F)c2)[C@H]1Cc1ccc(-c2cnco2)cc1]

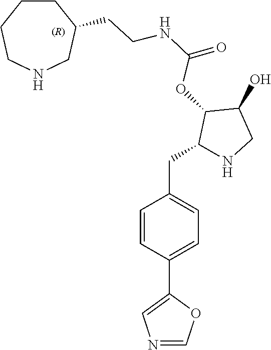

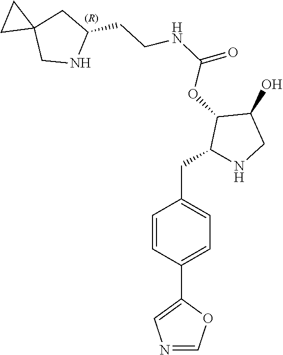

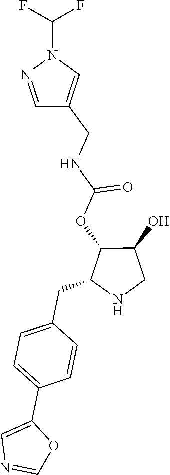 [SMILES: O=C(NCc1cnn(C(F)F)c1)O[C@@H]1[C@@H](O)CN[C@@H]1Cc1ccc(-c2cnco2)cc1]

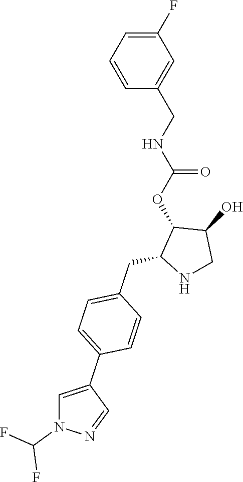 [SMILES: O=C(NCc1cccc(F)c1)O[C@@H]1[C@@H](O)CN[C@@H]1Cc1ccc(-c2cnn(C(F)F)c2)cc1]

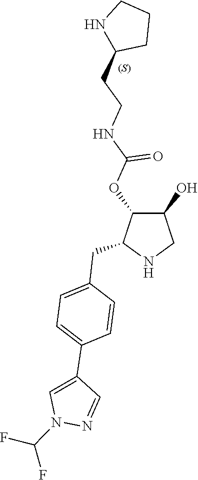

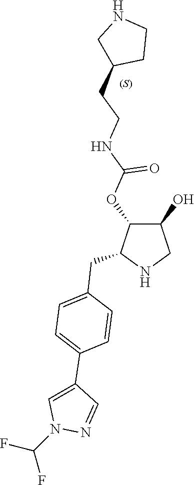

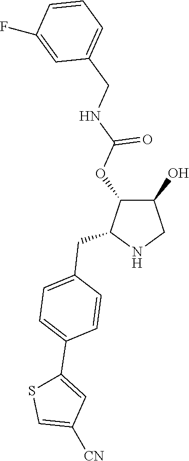 [SMILES: N#Cc1csc(-c2ccc(C[C@H]3NC[C@H](O)[C@H]3OC(=O)NCc3cccc(F)c3)cc2)c1]

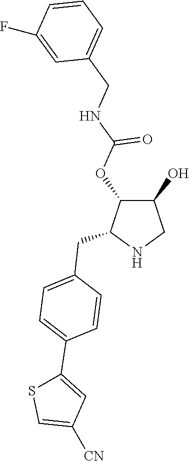 [SMILES: N#Cc1csc(-c2ccc(C[C@H]3NC[C@H](O)[C@H]3OC(=O)NCc3cccc(F)c3)cc2)c1]

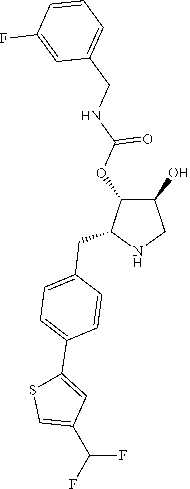 [SMILES: O=C(NCc1cccc(F)c1)O[C@@H]1[C@@H](O)CN[C@@H]1Cc1ccc(-c2cc(C(F)F)cs2)cc1]

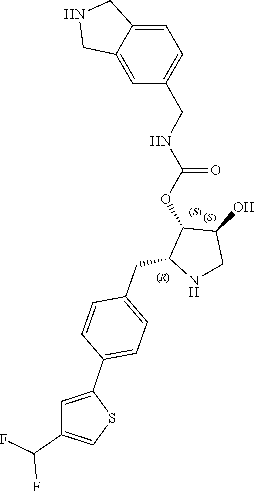

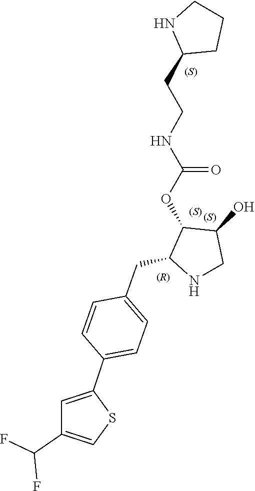

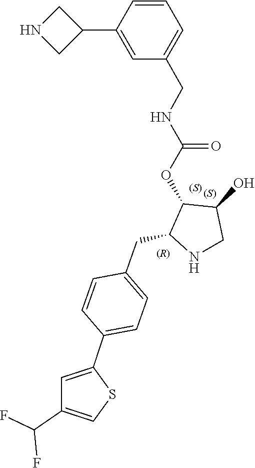

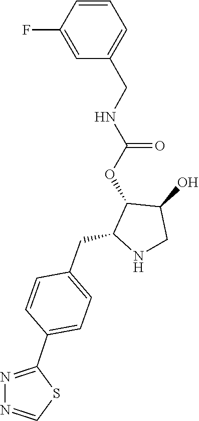 [SMILES: O=C(NCc1cccc(F)c1)O[C@@H]1[C@@H](O)CN[C@@H]1Cc1ccc(-c2nncs2)cc1]

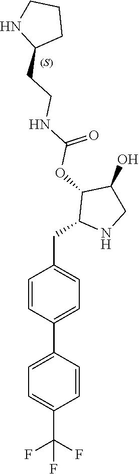

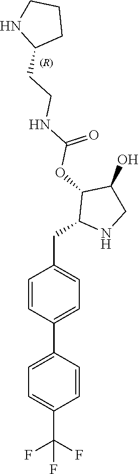

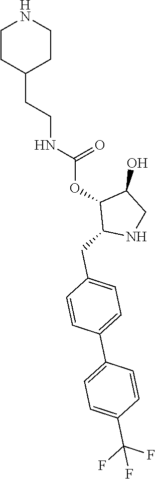 [SMILES: O=C(NCCC1CCNCC1)O[C@@H]1[C@@H](O)CN[C@@H]1Cc1ccc(-c2ccc(C(F)(F)F)cc2)cc1]

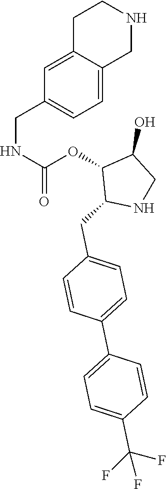 [SMILES: O=C(NCc1ccc2c(c1)CCNC2)O[C@@H]1[C@@H](O)CN[C@@H]1Cc1ccc(-c2ccc(C(F)(F)F)cc2)cc1]

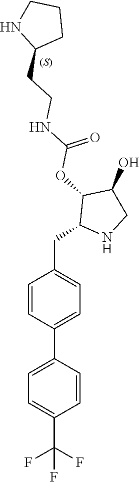

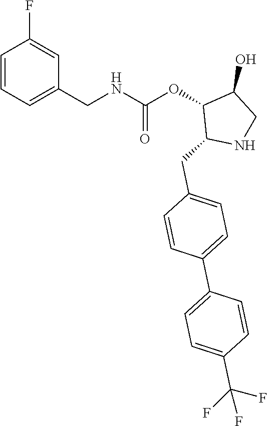 [SMILES: O=C(NCc1cccc(F)c1)O[C@@H]1[C@@H](O)CN[C@@H]1Cc1ccc(-c2ccc(C(F)(F)F)cc2)cc1]

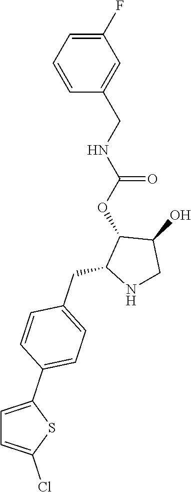 [SMILES: O=C(NCc1cccc(F)c1)O[C@@H]1[C@@H](O)CN[C@@H]1Cc1ccc(-c2ccc(Cl)s2)cc1]

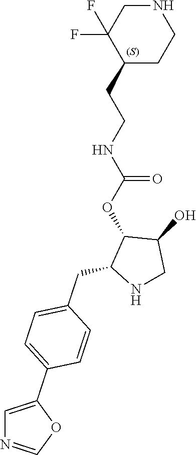

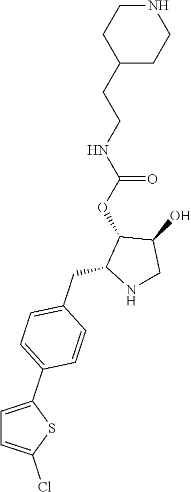 [SMILES: O=C(NCCC1CCNCC1)O[C@@H]1[C@@H](O)CN[C@@H]1Cc1ccc(-c2ccc(Cl)s2)cc1]

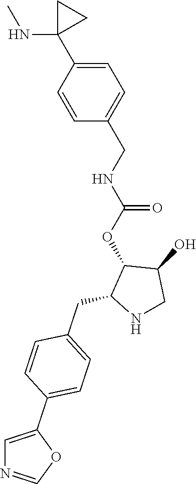 [SMILES: CNC1(c2ccc(CNC(=O)O[C@@H]3[C@@H](O)CN[C@@H]3Cc3ccc(-c4cnco4)cc3)cc2)CC1]

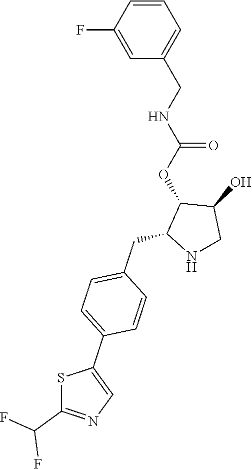 [SMILES: O=C(NCc1cccc(F)c1)O[C@@H]1[C@@H](O)CN[C@@H]1Cc1ccc(-c2cnc(C(F)F)s2)cc1]

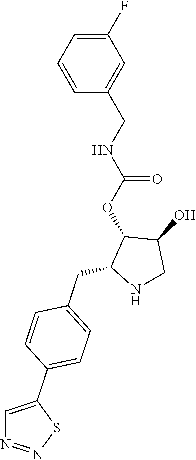 [SMILES: O=C(NCc1cccc(F)c1)O[C@@H]1[C@@H](O)CN[C@@H]1Cc1ccc(-c2cnns2)cc1]

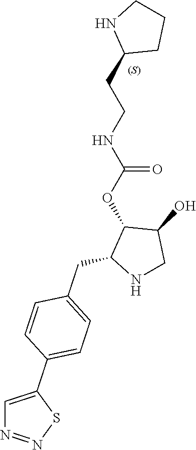

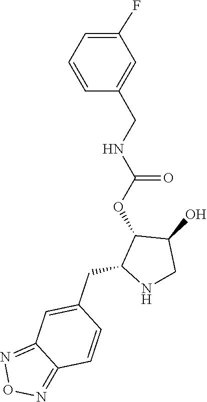 [SMILES: O=C(NCc1cccc(F)c1)O[C@@H]1[C@@H](O)CN[C@@H]1Cc1ccc2nonc2c1]

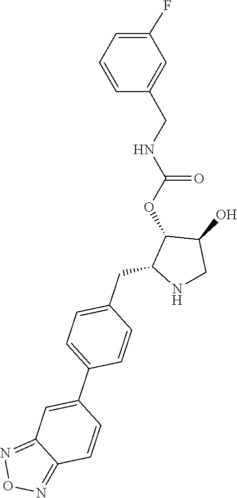 [SMILES: O=C(NCc1cccc(F)c1)O[C@@H]1[C@@H](O)CN[C@@H]1Cc1ccc(-c2ccc3nonc3c2)cc1]

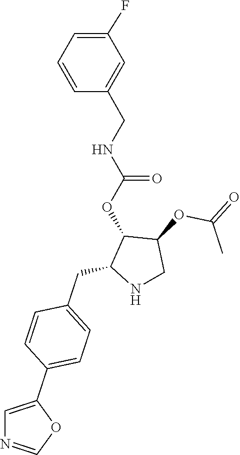 [SMILES: CC(=O)O[C@H]1CN[C@H](Cc2ccc(-c3cnco3)cc2)[C@@H]1OC(=O)NCc1cccc(F)c1]

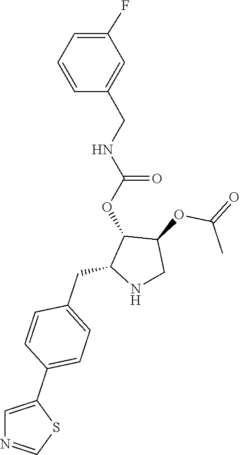 [SMILES: CC(=O)O[C@H]1CN[C@H](Cc2ccc(-c3cncs3)cc2)[C@@H]1OC(=O)NCc1cccc(F)c1]

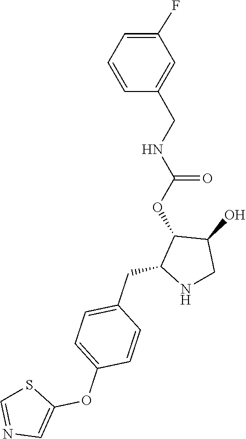 [SMILES: O=C(NCc1cccc(F)c1)O[C@@H]1[C@@H](O)CN[C@@H]1Cc1ccc(Oc2cncs2)cc1]

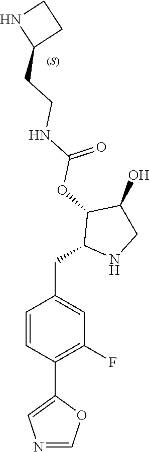

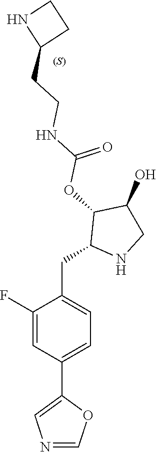

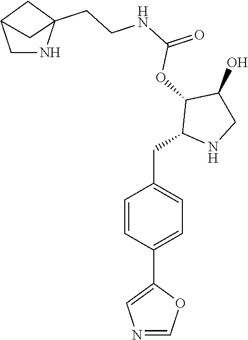 [SMILES: O=C(NCCC12CC(CN1)C2)O[C@@H]1[C@@H](O)CN[C@@H]1Cc1ccc(-c2cnco2)cc1]

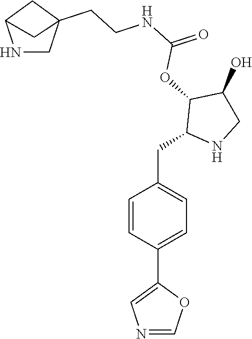 [SMILES: O=C(NCCC12CNC(C1)C2)O[C@@H]1[C@@H](O)CN[C@@H]1Cc1ccc(-c2cnco2)cc1]

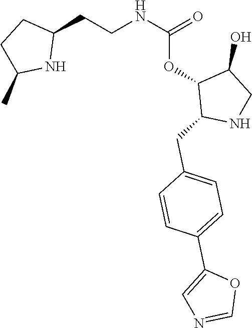 [SMILES: C[C@H]1CC[C@@H](CCNC(=O)O[C@@H]2[C@@H](O)CN[C@@H]2Cc2ccc(-c3cnco3)cc2)N1]

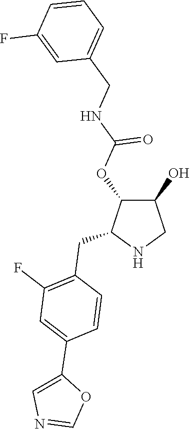 [SMILES: O=C(NCc1cccc(F)c1)O[C@@H]1[C@@H](O)CN[C@@H]1Cc1ccc(-c2cnco2)cc1F]

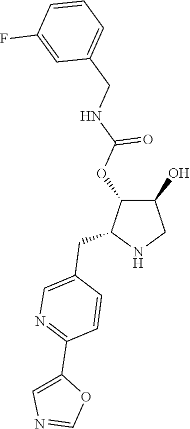 [SMILES: O=C(NCc1cccc(F)c1)O[C@@H]1[C@@H](O)CN[C@@H]1Cc1ccc(-c2cnco2)nc1]

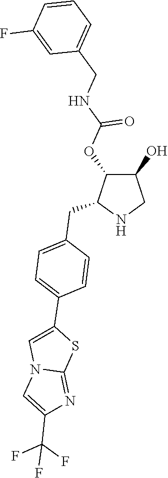 [SMILES: O=C(NCc1cccc(F)c1)O[C@@H]1[C@@H](O)CN[C@@H]1Cc1ccc(-c2cn3cc(C(F)(F)F)nc3s2)cc1]

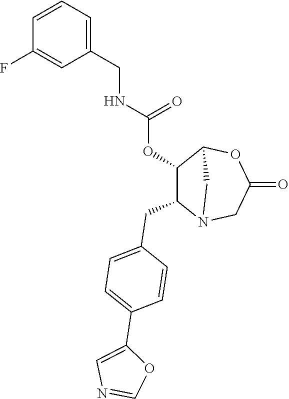 [SMILES: O=C1CN2C[C@H](O1)[C@@H](OC(=O)NCc1cccc(F)c1)[C@H]2Cc1ccc(-c2cnco2)cc1]

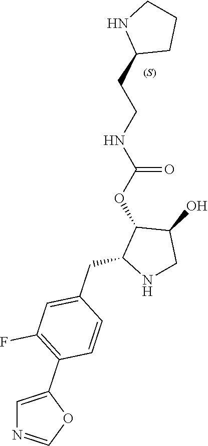

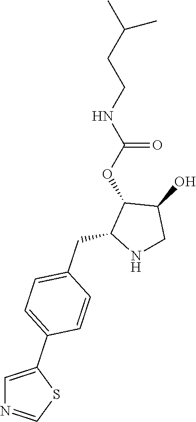 [SMILES: CC(C)CCNC(=O)O[C@@H]1[C@@H](O)CN[C@@H]1Cc1ccc(-c2cncs2)cc1]

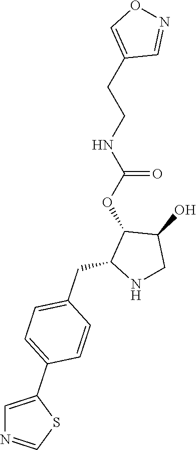 [SMILES: O=C(NCCc1cnoc1)O[C@@H]1[C@@H](O)CN[C@@H]1Cc1ccc(-c2cncs2)cc1]

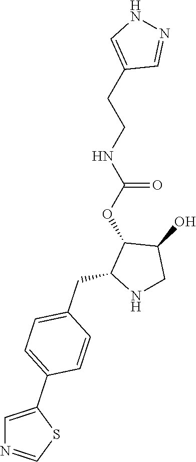 [SMILES: O=C(NCCc1cn[nH]c1)O[C@@H]1[C@@H](O)CN[C@@H]1Cc1ccc(-c2cncs2)cc1]

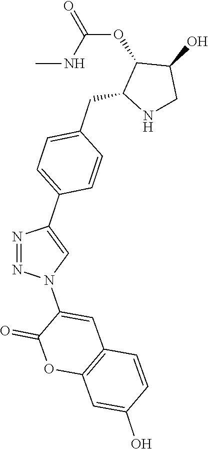 [SMILES: CNC(=O)O[C@@H]1[C@@H](O)CN[C@@H]1Cc1ccc(-c2cn(-c3cc4ccc(O)cc4oc3=O)nn2)cc1]

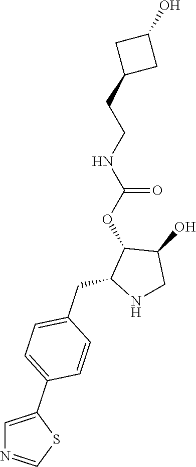 [SMILES: O=C(NCC[C@H]1C[C@H](O)C1)O[C@@H]1[C@@H](O)CN[C@@H]1Cc1ccc(-c2cncs2)cc1]

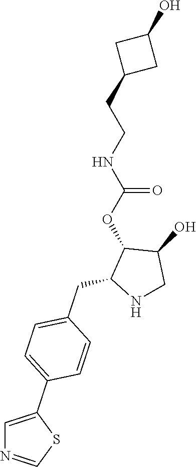 [SMILES: O=C(NCC[C@H]1C[C@@H](O)C1)O[C@@H]1[C@@H](O)CN[C@@H]1Cc1ccc(-c2cncs2)cc1]

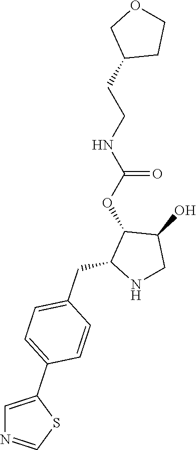 [SMILES: O=C(NCC[C@H]1CCOC1)O[C@@H]1[C@@H](O)CN[C@@H]1Cc1ccc(-c2cncs2)cc1]

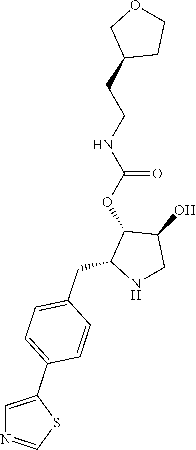 [SMILES: O=C(NCC[C@@H]1CCOC1)O[C@@H]1[C@@H](O)CN[C@@H]1Cc1ccc(-c2cncs2)cc1]

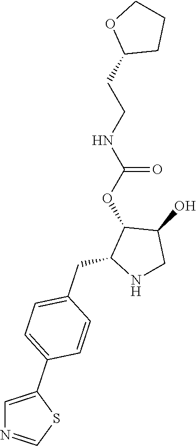 [SMILES: O=C(NCC[C@H]1CCCO1)O[C@@H]1[C@@H](O)CN[C@@H]1Cc1ccc(-c2cncs2)cc1]

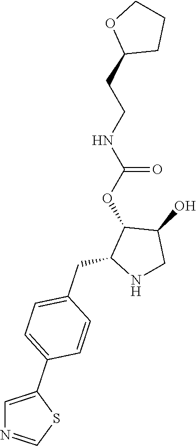 [SMILES: O=C(NCC[C@@H]1CCCO1)O[C@@H]1[C@@H](O)CN[C@@H]1Cc1ccc(-c2cncs2)cc1]

In some embodiments, the compound of Formula (V), or pharmaceutically acceptable salt thereof, is selected from those in Table 2, and pharmaceutically acceptable salts thereof:

TABLE 2
Compounds of Formula (V)

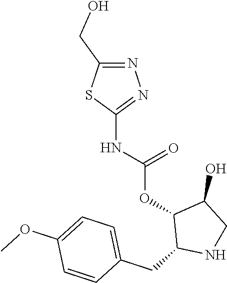 [SMILES: COc1ccc(C[C@H]2NC[C@H](O)[C@H]2OC(=O)Nc2nnc(CO)s2)cc1]

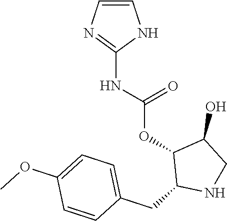 [SMILES: COc1ccc(C[C@H]2NC[C@H](O)[C@H]2OC(=O)Nc2ncc[nH]2)cc1]

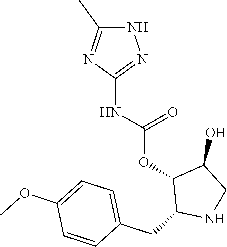 [SMILES: COc1ccc(C[C@H]2NC[C@H](O)[C@H]2OC(=O)Nc2n[nH]c(C)n2)cc1]

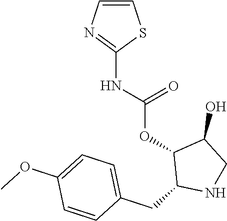 [SMILES: COc1ccc(C[C@H]2NC[C@H](O)[C@H]2OC(=O)Nc2nccs2)cc1]

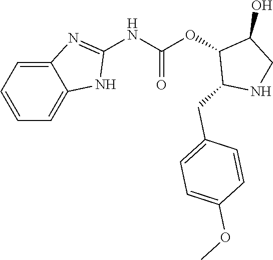 [SMILES: COc1ccc(C[C@H]2NC[C@H](O)[C@H]2OC(=O)Nc2nc3ccccc3[nH]2)cc1]

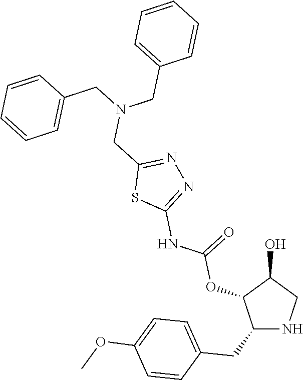 [SMILES: COc1ccc(C[C@H]2NC[C@H](O)[C@H]2OC(=O)Nc2nnc(CN(Cc3ccccc3)Cc3ccccc3)s2)cc1]

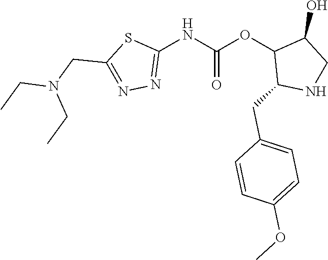 [SMILES: CCN(CC)Cc1nnc(NC(=O)OC2[C@@H](O)CN[C@@H]2Cc2ccc(OC)cc2)s1]

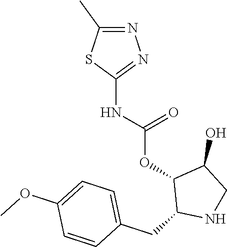 [SMILES: COc1ccc(C[C@H]2NC[C@H](O)[C@H]2OC(=O)Nc2nnc(C)s2)cc1]

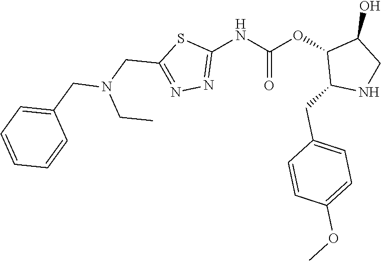 [SMILES: CCN(Cc1ccccc1)Cc1nnc(NC(=O)O[C@@H]2[C@@H](O)CN[C@@H]2Cc2ccc(OC)cc2)s1]

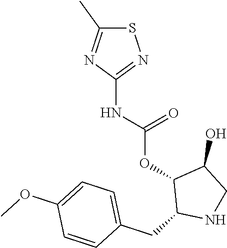 [SMILES: COc1ccc(C[C@H]2NC[C@H](O)[C@H]2OC(=O)Nc2nsc(C)n2)cc1]

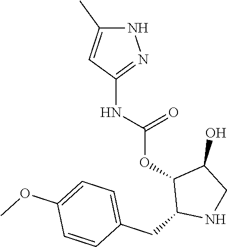 [SMILES: COc1ccc(C[C@H]2NC[C@H](O)[C@H]2OC(=O)Nc2cc(C)[nH]n2)cc1]

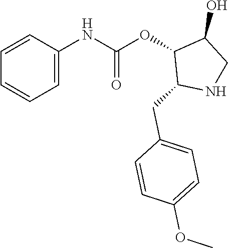 [SMILES: COc1ccc(C[C@H]2NC[C@H](O)[C@H]2OC(=O)Nc2ccccc2)cc1]

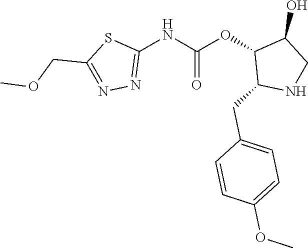 [SMILES: COCc1nnc(NC(=O)O[C@@H]2[C@@H](O)CN[C@@H]2Cc2ccc(OC)cc2)s1]

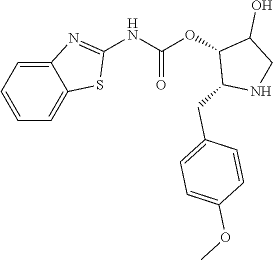 [SMILES: COc1ccc(C[C@H]2NCC(O)[C@H]2OC(=O)Nc2nc3ccccc3s2)cc1]

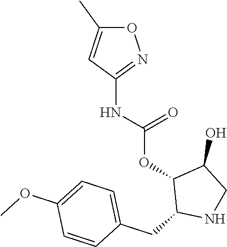 [SMILES: COc1ccc(C[C@H]2NC[C@H](O)[C@H]2OC(=O)Nc2cc(C)on2)cc1]

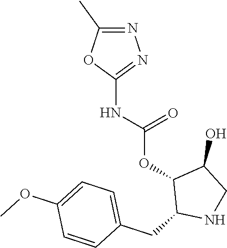 [SMILES: COc1ccc(C[C@H]2NC[C@H](O)[C@H]2OC(=O)Nc2nnc(C)o2)cc1]

In some embodiments, a provided compound (a compound described herein, a compound of the present disclosure) is a compound of any of the formulae herein (e.g., Formulae (I), (II), (III), (IV), (V)) or pharmaceutically acceptable salt thereof. In some embodiments, a provided compound is a compound of any of the formulae herein (e.g., Formulae (I), (II), (III), (IV), (V)), or a salt thereof. In some embodiments, a provided compound is a compound of any of the formulae herein (e.g., Formulae (I), (II), (III), (IV), (V)).

### Pharmaceutical Compositions, Kits, and Administration

The present disclosure provides pharmaceutical compositions comprising a compound provided herein, or a pharmaceutically acceptable salt thereof (e.g., a compound of Formula (I), or a pharmaceutically acceptable salt thereof), and optionally a pharmaceutically acceptable excipient. In some embodiments, the pharmaceutical composition described herein comprises a compound provided herein, or a pharmaceutically acceptable salt thereof, and a pharmaceutically acceptable excipient. In certain embodiments, a compound described herein is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable carrier or excipient, and/or any additional ingredients in a pharmaceutical composition described herein will vary, depending upon the identity, size, and/or condition of the subject treated and further depending upon the route by which the composition is to be administered.

Pharmaceutically acceptable carriers/excipients used in the manufacture of provided pharmaceutical compositions include inert diluents, solvents, dispersing and/or granulating agents, surface active agents and/or emulsifiers, disintegrating agents, binding agents, preservatives, buffering agents, lubricating agents, oils, butters, and/or waxes. Excipients such as coloring agents, coating agents, sweetening agents, flavoring agents, and fragrances may also be present in the composition.

The compounds and compositions provided herein can be administered by any route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form a single unit dosage form. Thus, in one aspect, provided are kits including a first container comprising a compound or pharmaceutical composition described herein. In certain embodiments, the kits are useful for treating and/or preventing a disease, disorder, or condition in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits provide instructions for treating a disease (e.g., cancer) in a subject in need thereof. In certain embodiments, the kits provide instructions for preventing a disease in a subject in need thereof. A kit described herein may include one or more additional pharmaceutical agents described herein as a separate composition.

### Methods of Modulating Protein Synthesis

In another aspect, the present disclosure provides a method of modulating protein synthesis in a subject in need thereof or in a cell, tissue, or biological sample, comprising administering to the subject in need thereof or contacting the cell, tissue, or biological sample with an effective amount of a provided compound (e.g., a compound of the present disclosure, or a pharmaceutically acceptable salt thereof), or a pharmaceutical composition thereof. In another aspect, the present disclosure provides a method of modulating protein synthesis in a subject in need thereof or in a cell, tissue, or biological sample, comprising administering to the subject in need thereof or contacting the cell, tissue, or biological sample with an effective amount of a compound of Formula (I), or a pharmaceutical composition thereof. In another aspect, the present disclosure provides a method of modulating protein synthesis in a subject in need thereof or in a cell, tissue, or biological sample, comprising administering to the subject in need thereof or contacting the cell, tissue, or biological sample with an effective amount of a compound of Formula (V), or a pharmaceutical composition thereof.

In some embodiments, the present disclosure provides a method of modulating protein synthesis in a subject in need thereof, comprising administering to the subject in need thereof an effective amount of a provided compound, or a pharmaceutical composition thereof. In some embodiments, the present disclosure provides a method of modulating protein synthesis in a cell, tissue, or biological sample, comprising contacting the cell, tissue, or biological sample with an effective amount of a provided compound, or a pharmaceutical composition thereof. In some embodiments, the present disclosure provides a provided compound, or a pharmaceutical composition thereof, for use in modulating protein synthesis in a subject in need thereof. In some embodiments, the present disclosure provides a provided compound, or a pharmaceutical composition thereof, for use in the manufacture of a medicament for modulating protein synthesis in a subject in need thereof.

In some embodiments modulating protein synthesis comprises modulating synthesis of a target protein. In some embodiments, modulating protein synthesis comprises decreasing protein synthesis. In some embodiments, the protein synthesis is decreased by at least about 10%, at least about 20%, at least about 30%, at least about 40%, at least about 50%, at least about 60%, at least about 70%, at least about 80%, at least about 90%, at least about 95%, or at least about 98%. In some embodiments, protein synthesis is decreased by not more than about 10%, not more than about 20%, not more than about 30%, not more than about 40%, not more than about 50%, not more than about 60%, not more than about 70%, not more than about 80%, not more than about 90%, not more than about 95%, or not more than about 98%. In some embodiments, protein synthesis is decreased by a range between a percentage described in this paragraph and another percentage described in this paragraph, inclusive.

In another aspect, the present disclosure provides a method of decreasing protein synthesis in a subject in need thereof or in a cell, tissue, or biological sample, comprising administering to the subject in need thereof or contacting the cell, tissue, or biological sample with an effective amount of a provided compound (e.g., a compound of the present disclosure, or a pharmaceutically acceptable salt thereof), or a pharmaceutical composition thereof. In some embodiments, the present disclosure provides a method of decreasing protein synthesis in a subject in need thereof, comprising administering to the subject in need thereof an effective amount of a provided compound, or a pharmaceutical composition thereof. In some embodiments, the present disclosure provides a method of decreasing protein synthesis in a cell, tissue, or biological sample, comprising contacting the cell, tissue, or biological sample with an effective amount of a provided compound, or a pharmaceutical composition thereof. In some embodiments, the present disclosure provides a provided compound, or a pharmaceutical composition thereof, for use in decreasing protein synthesis in a subject in need thereof. In some embodiments, the present disclosure provides a provided compound, or a pharmaceutical composition thereof, for use in the manufacture of a medicament for decreasing protein synthesis in a subject in need thereof.

In some embodiments, decreasing protein synthesis comprises decreasing synthesis of a target protein. In some embodiments, the protein synthesis is decreased by at least about 10%, at least about 20%, at least about 30%, at least about 40%, at least about 50%, at least about 60%, at least about 70%, at least about 80%, at least about 90%, at least about 95%, or at least about 98%. In some embodiments, protein synthesis is decreased by not more than about 10%, not more than about 20%, not more than about 30%, not more than about 40%, not more than about 50%, not more than about 60%, not more than about 70%, not more than about 80%, not more than about 90%, not more than about 95%, or not more than about 98%. In some embodiments, protein synthesis is decreased by a range between a percentage described in this paragraph and another percentage described in this paragraph, inclusive.

In some embodiments, the method is selective for decreasing synthesis of a first protein compared to synthesis of a second protein. In some embodiments, the ratio of the decrease in synthesis of the first protein to the decrease in synthesis of the second protein is about 1.1:1, about 1.2:1, about 1.3:1, about 1.4:1, about 1.5:1, about 1.6:1, about 1.7:1, about 1.8:1, about 1.9:1, about 2:1, about 2.5:1, about 3:1, about 3.5:1, about 4:1, about 4.5:1, about 5:1, about 6:1, about 7:1, about 8:1, about 9:1, about 10:1, about 15:1, about 20:1, about 25:1, about 50:1, about 75:1, about 100:1, about 200:1, about 300:1, about 400:1, about 500:1, about 1,000:1, about 10,000:1, or about 100,000:1. In some embodiments, the ratio of the decrease in synthesis of the first protein to the decrease in synthesis of the second protein is between a ratio described in this paragraph and another ratio described in this paragraph, inclusive.

In some embodiments, the method further comprises decreasing an amount of mRNA, wherein the mRNA is associated with synthesis of the target protein. In some embodiments, the amount of mRNA is decreased by at least about 10%, at least about 20%, at least about 30%, at least about 40%, at least about 50%, at least about 60%, at least about 70%, at least about 80%, at least about 90%, at least about 95%, or at least about 98%. In some embodiments, the amount of mRNA is decreased by not more than 10%, not more than 20%, not more than 30%, not more than 40%, not more than 50%, not more than 60%, not more than 70%, not more than 80%, not more than 90%, not more than 95%, or not more than 98%. In some embodiments, the amount of mRNA is decreased by a range between a percentage described in this paragraph and another percentage described in this paragraph, inclusive.

In some embodiments, the method is in vitro. In some embodiments, the method is in vivo.

In some embodiments, the target protein is B-cell lymphoma 2 (BCL-2), MYC proto-oncogene bHLH transcription factor (MYC), cyclin D1 (CCND1), myeloid cell leukemia 1 (MCL-1), anaplastic lymphoma kinase (ALK), or GTPase KRas G12D mutant (KRAS-G12D). In some embodiments, the target protein is BCL-2. In some embodiments, the target protein is MYC. In some embodiments, the target protein is CCND1. In some embodiments, the target protein is MCL-1. In some embodiments, the target protein is ALK. In some embodiments, the target protein is KRAS-G12D. The target proteins KRAS-G12D. BCL-2, MYC, CCND1, MCL-1, ALK, and KRAS-G12D are exemplary target proteins, and the methods disclosed herein are not limited to these target proteins.

In some embodiments, the cell is a cancer cell. In some embodiments, the cell is a HEK 293T, HPAF-II, KLE, LS411N, MCF7, NCI-H1915, HCC38, HEPG2, KATO-III, MS751, or T47D cell. In some embodiments, the cell is a HEK 293T cell. In some embodiments, the cell is a HPAF-II cell. In some embodiments, the cell is a KLE cell. In some embodiments, the cell is a LS411N cell. In some embodiments, the cell is a MCF7 cell. In some embodiments, the cell is a NCI-H1915 cell. In some embodiments, the cell is a HCC38 cell. In some embodiments, the cell is a HEPG2 cell. In some embodiments, the cell is a KATO-III cell. In some embodiments, the cell is a MS751 cell. In some embodiments, the cell is a T47D cell.

### Methods of Treatment and Prevention

In another aspect, the present disclosure provides a method of treating or preventing a disease in a subject in need thereof, comprising administering to the subject in need thereof a therapeutically effective amount of a provided compound (e.g., a compound of the present disclosure, or a pharmaceutically acceptable salt thereof), or a pharmaceutical composition thereof. In some embodiments, the present disclosure provides a method of treating a disease in a subject in need thereof, comprising administering to the subject in need thereof a therapeutically effective amount of a provided compound, or a pharmaceutical composition thereof. In some embodiments, the present disclosure provides a method of preventing a disease in a subject in need thereof, comprising administering to the subject in need thereof a therapeutically effective amount of a provided compound, or a pharmaceutical composition thereof. In another aspect, the present disclosure provides a method of treating or preventing a disease in a subject in need thereof, comprising administering to the subject in need thereof a therapeutically effective amount of a compound of Formula (I), or a pharmaceutical composition thereof. In another aspect, the present disclosure provides a method of treating or preventing a disease in a subject in need thereof, comprising administering to the subject in need thereof a therapeutically effective amount of a compound of Formula (V), or a pharmaceutical composition thereof.

In some embodiments, the present disclosure provides a provided compound, or a pharmaceutical composition thereof, for use in treating or preventing a disease in a subject in need thereof. In some embodiments, the present disclosure provides a provided compound, or a pharmaceutical composition thereof, for use in treating a disease in a subject in need thereof. In some embodiments, the present disclosure provides a provided compound, or a pharmaceutical composition thereof, for use in preventing a disease in a subject in need thereof.

In some embodiments, the present disclosure provides a provided compound, or a pharmaceutical composition thereof, for use in the manufacture of a medicament for treating or preventing a disease in a subject in need thereof. In some embodiments, the present disclosure provides a provided compound, or a pharmaceutical composition thereof, for use in the manufacture of a medicament for treating a disease in a subject in need thereof. In some embodiments, the present disclosure provides a provided compound, or a pharmaceutical composition thereof, for use in the manufacture of a medicament for preventing a disease in a subject in need thereof.

In some embodiments, the disease is a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis).

In some embodiments, the disease is a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)). In some embodiments, the proliferative disease is cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma). In some embodiments, the cancer is prostate cancer (e.g., prostate adenocarcinoma), pancreatic cancer (e.g., pancreatic adenocarcinoma, intraductal papillary mucinous neoplasm (IPMN), Islet cell tumors), lung cancer (e.g., bronchogenic carcinoma, small cell lung cancer (SCLC), non-small cell lung cancer (NSCLC), adenocarcinoma of the lung), breast cancer (e.g., adenocarcinoma of the breast, papillary carcinoma of the breast, mammary cancer, medullary carcinoma of the breast, triple-negative breast cancer (TNBC)), colorectal cancer (e.g., colon cancer, rectal cancer, colorectal adenocarcinoma), endometrial cancer (e.g., uterine cancer, uterine sarcoma), ovarian cancer (e.g., cystadenocarcinoma, ovarian embryonal carcinoma, ovarian adenocarcinoma), cervical cancer (e.g., cervical adenocarcinoma), esophageal cancer (e.g., adenocarcinoma of the esophagus, Barrett's adenocarcinoma), bladder cancer, biliary cancer (e.g., cholangiocarcinoma), hematopoietic cancer (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), or neuroblastoma.

In some embodiments, the cancer is prostate cancer (e.g., prostate adenocarcinoma). In some embodiments, the cancer is prostate adenocarcinoma. In some embodiments, the cancer is pancreatic cancer (e.g., pancreatic adenocarcinoma, intraductal papillary mucinous neoplasm (IPMN), Islet cell tumors). In some embodiments, the cancer is pancreatic adenocarcinoma. In some embodiments, the cancer is intraductal papillary mucinous neoplasm (IPMN). In some embodiments, the cancer is Islet cell tumors. In some embodiments, the cancer is lung cancer (e.g., bronchogenic carcinoma, small cell lung cancer (SCLC), non-small cell lung cancer (NSCLC), adenocarcinoma of the lung). In some embodiments, the cancer is bronchogenic carcinoma. In some embodiments, the cancer is small cell lung cancer (SCLC). In some embodiments, the cancer is non-small cell lung cancer (NSCLC). In some embodiments, the cancer is adenocarcinoma of the lung. In some embodiments, the cancer is breast cancer (e.g., adenocarcinoma of the breast, papillary carcinoma of the breast, mammary cancer, medullary carcinoma of the breast, triple-negative breast cancer (TNBC)). In some embodiments, the cancer is adenocarcinoma of the breast. In some embodiments, the cancer is papillary carcinoma of the breast. In some embodiments, the cancer is mammary cancer. In some embodiments, the cancer is medullary carcinoma of the breast. In some embodiments, the cancer is triple-negative breast cancer (TNBC). In some embodiments, the cancer is colorectal cancer (e.g., colon cancer, rectal cancer, colorectal adenocarcinoma). In some embodiments, the cancer is colon cancer. In some embodiments, the cancer is rectal cancer. In some embodiments, the cancer is colorectal adenocarcinoma. In some embodiments, the cancer is endometrial cancer (e.g., uterine cancer, uterine sarcoma). In some embodiments, the cancer is uterine cancer. In some embodiments, the cancer is uterine sarcoma. In some embodiments, the cancer is ovarian cancer (e.g., cystadenocarcinoma, ovarian embryonal carcinoma, ovarian adenocarcinoma). In some embodiments, the cancer is cystadenocarcinoma. In some embodiments, the cancer is ovarian embryonal carcinoma. In some embodiments, the cancer is ovarian adenocarcinoma. In some embodiments, the cancer is cervical cancer (e.g., cervical adenocarcinoma). In some embodiments, the cancer is cervical adenocarcinoma. In some embodiments, the cancer is esophageal cancer (e.g., adenocarcinoma of the esophagus, Barrett's adenocarcinoma). In some embodiments, the cancer is adenocarcinoma of the esophagus. In some embodiments, the cancer is Barrett's adenocarcinoma. In some embodiments, the cancer is bladder cancer. In some embodiments, the cancer is biliary cancer (e.g., cholangiocarcinoma). In some embodiments, the cancer is cholangiocarcinoma. In some embodiments, the cancer is hematopoietic cancer (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)). In some embodiments, the cancer is leukemia (e.g., acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)). In some embodiments, the cancer is acute lymphocytic leukemia (ALL) (e.g., B-cell ALL, T-cell ALL). In some embodiments, the cancer is B-cell ALL. In some embodiments, the cancer is T-cell ALL. In some embodiments, the cancer is acute myelocytic leukemia (AML) (e.g., B-cell AML, T-cell AML). In some embodiments, the cancer is B-cell AML. In some embodiments, the cancer is T-cell AML. In some embodiments, the cancer is chronic myelocytic leukemia (CML) (e.g., B-cell CML, T-cell CML). In some embodiments, the cancer is B-cell CML. In some embodiments, the cancer is T-cell CML. In some embodiments, the cancer is chronic lymphocytic leukemia (CLL) (e.g., B-cell CLL, T-cell CLL). In some embodiments, the cancer is B-cell CLL. In some embodiments, the cancer is T-cell CLL. In some embodiments, the cancer is lymphoma (e.g., Hodgkin lymphoma (HL) (e.g., B-cell HL, T-cell HL); non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma); T-cell NHL (e.g., precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma)). In some embodiments, the cancer is Hodgkin lymphoma (HL) (e.g., B-cell HL, T-cell HL). In some embodiments, the cancer is B-cell HL. In some embodiments, the cancer is T-cell HL. In some embodiments, the cancer is non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma). In some embodiments, the cancer is diffuse large cell lymphoma (DLCL) (e.g., diffuse large B-cell lymphoma). In some embodiments, the cancer is diffuse large B-cell lymphoma. In some embodiments, the cancer is follicular lymphoma. In some embodiments, the cancer is chronic lymphocytic leukemia/small lymphocytic lymphoma (CLL/SLL). In some embodiments, the cancer is mantle cell lymphoma (MCL). In some embodiments, the cancer is marginal zone B-cell lymphomas (e.g., mucosa-associated lymphoid tissue (MALT) lymphomas, nodal marginal zone B-cell lymphoma, splenic marginal zone B-cell lymphoma). In some embodiments, the cancer is mucosa-associated lymphoid tissue (MALT) lymphomas. In some embodiments, the cancer is nodal marginal zone B-cell lymphoma. In some embodiments, the cancer is splenic marginal zone B-cell lymphoma. In some embodiments, the cancer is primary mediastinal B-cell lymphoma. In some embodiments, the cancer is Burkitt lymphoma. In some embodiments, the cancer is lymphoplasmacytic lymphoma (i.e., Waldenström's macroglobulinemia). In some embodiments, the cancer is Waldenström's macroglobulinemia. In some embodiments, the cancer is hairy cell leukemia (HCL). In some embodiments, the cancer is immunoblastic large cell lymphoma. In some embodiments, the cancer is precursor B-lymphoblastic lymphoma. In some embodiments, the cancer is primary central nervous system (CNS) lymphoma. In some embodiments, the cancer is T-cell NHL (e.g., precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma)). In some embodiments, the cancer is precursor T-lymphoblastic lymphoma/leukemia. In some embodiments, the cancer is peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma). In some embodiments, the cancer is cutaneous T-cell lymphoma (CTCL) (e.g., mycosis fungoides, Sezary syndrome). In some embodiments, the cancer is mycosis fungoides. In some embodiments, the cancer is Sezary syndrome. In some embodiments, the cancer is angioimmunoblastic T-cell lymphoma. In some embodiments, the cancer is extranodal natural killer T-cell lymphoma. In some embodiments, the cancer is enteropathy type T-cell lymphoma. In some embodiments, the cancer is subcutaneous panniculitis-like T-cell lymphoma. In some embodiments, the cancer is anaplastic large cell lymphoma. In some embodiments, the cancer is a mixture of one or more leukemia/lymphoma as described above. In some embodiments, the cancer is multiple myeloma (MM). In some embodiments, the cancer is neuroblastoma.

In some embodiments, the disease is a neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)).

In some embodiments, the neurological disease is cerebellar ataxia. In some embodiments, the neurological disease is a neurodegenerative disease (e.g., Alzheimer's disease, Parkinson's disease, amyotrophic lateral sclerosis, tauopathy (including frontotemporal dementia), Huntington's disease, Friedreich's ataxia). In some embodiments, the neurodegenerative disease is Alzheimer's disease. In some embodiments, the neurodegenerative disease is Parkinson's disease. In some embodiments, the neurodegenerative disease is amyotrophic lateral sclerosis. In some embodiments, the neurodegenerative disease is tauopathy (e.g., frontotemporal dementia). In some embodiments, the neurodegenerative disease is frontotemporal dementia. In some embodiments, the neurodegenerative disease is Huntington's disease. In some embodiments, the neurodegenerative disease is Friedreich's ataxia.

In some embodiments, the disease is an immune disorder (e.g., psoriasis, lupus, rheumatoid arthritis). In some embodiments, the immune disorder is psoriasis. In some embodiments, the immune disorder is lupus. In some embodiments, the immune disorder is rheumatoid arthritis.

In some embodiments, the disease is associated with BCL-2, MYC, CCND1, MCL-1, ALK, or KRAS-G12D. In some embodiments, the disease is associated with MYC, ALK, or KRAS-G12D. In some embodiments, the disease associated with BCL-2, MYC, CCND1, MCL-1, ALK, or KRAS-G12D is a proliferative disease (e.g., cancer (e.g., prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, neuroblastoma)), neurological disease (e.g., cerebellar ataxia, neurodegenerative disease (e.g., Alzheimer's disease, Parkinson's disease, amyotrophic lateral sclerosis, tauopathy (including frontotemporal dementia), Huntington's disease, Friedreich's ataxia)), or immune disorder (e.g., psoriasis, lupus, rheumatoid arthritis).

In certain embodiments, the disease or disorder is cancer. In some embodiments, the cancer is breast cancer, lung cancer, prostate cancer, bladder cancer, liver cancer, colorectal cancer, endometrial cancer, ovarian cancer, pancreatic cancer, esophagus cancer, gastric cancer, esophageal cancer, uterine cancer, skin cancer, leukemia, or lymphoma.

In some embodiments, the disease or disorder is Breast cancer, NSCLC, Prostate cancer, Bladder cancer, Colorectal cancer, Endometrial cancer, Melanoma, Ovarian cancer, Pancreatic cancer, Hepatocellular cancer, Esophagus cancer, Gastric cancer, Diffuse Large B-cell lymphoma, Uterine sarcoma, or Acute myeloid leukemia.

In certain embodiments, the disease or disorder is Acral Lentiginous Melanoma, Acute Lymphoblastic Leukemia, Acute Myeloid Leukemia, Adenocarcinoma of the Gastroesophageal Junction, AL Amyloidosis, ALK-Positive Anaplastic Large Cell Lymphoma, ALK-Positive Large B-Cell Lymphoma, Anal Carcinoma, Anaplastic Large Cell Lymphoma, Astrocytoma, B-Cell Acute Lymphoblastic Leukemia, B-Cell Lymphoma, B-Cell Non-Hodgkin Lymphoma, Biliary Tract Carcinoma, Bladder Carcinoma, Bladder Papillary Urothelial Neoplasm, Brain Glioblastoma, Breast Angiosarcoma, Breast Carcinoma, Bronchogenic Carcinoma, Burkitt Lymphoma, Carcinoma, Central Nervous System Neoplasm, Cholangiocarcinoma, Chondrosarcoma, Chronic Lymphocytic Leukemia, Chronic Lymphocytic Leukemia/Small Lymphocytic Lymphoma, Chronic Myeloid Leukemia, Colon Carcinoma, Colorectal Adenocarcinoma, Colorectal Carcinoma, Dedifferentiated Chondrosarcoma, Desmoid-Type Fibromatosis, Desmoplastic/Nodular Medulloblastoma, Diffuse Large B-Cell Lymphoma, Diffuse Large B-Cell Lymphoma Activated B-Cell Type, Double-Hit Lymphoma, EBV-Positive Diffuse Large B-Cell Lymphoma, Endometrial Serous Adenocarcinoma, Erdheim-Chester Disease, Esophageal Adenocarcinoma, Esophageal Adenosquamous Carcinoma, Esophageal Carcinoma, Esophageal Squamous Cell Carcinoma, Ewing Sarcoma, Follicular Lymphoma, Ganglioneuroblastoma, Gastric Adenocarcinoma, Gastric Adenosquamous Carcinoma, Gastric Carcinoma, Gastric Squamous Cell Carcinoma, Germinal Center B-Cell-Like Diffuse Large B-Cell Lymphoma, Glioblastoma, Hairy Cell Leukemia, Head and Neck Carcinoma, Head and Neck Squamous Cell Carcinoma, Hematopoietic and Lymphoid Malignancy, Hepatocellular Carcinoma, High Grade B-Cell Lymphoma, High Grade B-Cell Lymphoma with MYC and BCL2 and/or BCL6 Rearrangements, High Grade Ovarian Serous Adenocarcinoma, Histiocytic and Dendritic Cell Neoplasm, Hodgkin Lymphoma, Hypopharyngeal Squamous Cell Carcinoma, Inflammatory Myofibroblastic Tumor, Intracranial Primitive Neuroectodermal Neoplasm, Intrahepatic Cholangiocarcinoma, Intraocular Lymphoma, Invasive Breast Carcinoma, Juvenile Myelomonocytic Leukemia, Langerhans Cell Histiocytosis, Large Cell/Anaplastic Medulloblastoma, Laryngeal Squamous Cell Carcinoma, Leukemia, Low Grade Glioma, Lung Adenocarcinoma, Lung Carcinoma, Lymphoma, Lymphoplasmacytic Lymphoma, Malignant Breast Neoplasm, Malignant Central Nervous System Neoplasm, Malignant Colon Neoplasm, Malignant Colorectal Neoplasm, Malignant Endometrial Neoplasm, Malignant Gastric Neoplasm, Malignant Glioma, Malignant Lung Neoplasm, Malignant Ovarian Epithelial Tumor, Malignant Ovarian Neoplasm, Malignant Pancreatic Neoplasm, Malignant Pleural Mesothelioma, Malignant Prostate Neoplasm, Malignant Solid Tumor, Malignant Thyroid Gland Neoplasm, Mantle Cell Lymphoma, Marginal Zone Lymphoma, Mature B-Cell Lymphoma/Leukemia, Mature B-Cell Non-Hodgkin Lymphoma, Mature T-Cell and NK-Cell Lymphoma/Leukemia, Medulloblastoma, Medulloblastoma with Extensive Nodularity, Melanoma, Merkel Cell Carcinoma, Multiple Myeloma, Myelodysplastic/Myeloproliferative Neoplasm, Myeloid Neoplasm, Nasopharyngeal Carcinoma, Neuroblastoma, Non-Hodgkin Lymphoma, Non-Small Cell Lung Carcinoma, Non-Squamous Non-Small Cell Lung Carcinoma, Oral Cavity Carcinoma, Oropharyngeal Squamous Cell Carcinoma, Osteosarcoma, Ovarian Carcinoma, Pancreatic Adenocarcinoma, Pancreatic Carcinoma, Pancreatic Ductal Adenocarcinoma, Penile Carcinoma, Peripheral T-Cell Lymphoma, Primary Central Nervous System Lymphoma, Primary Cutaneous Anaplastic Large Cell Lymphoma, Primary Malignant Liver Neoplasm, Prostate Carcinoma, Rosai-Dorfman Disease, Sarcoma, Small Cell Lung Carcinoma, Small Intestinal Carcinoma, Small Intestinal Lymphoma, Small Lymphocytic Leukemia, Small Lymphocytic Lymphoma, Soft Tissue Sarcoma, Squamous Cell Lung Carcinoma, Synovial Sarcoma, Systemic Anaplastic Large Cell Lymphoma, Thyroid Gland Follicular Carcinoma, Thyroid Gland Medullary Carcinoma, Thyroid Gland Undifferentiated (Anaplastic) Carcinoma, Transformed Non-Hodgkin Lymphoma, Triple-Hit Lymphoma, Urothelial Carcinoma, Vaginal Carcinoma, or Vulvar Carcinoma.

In some embodiments, the disease or disorder is associated with KRAS-G12D. In some embodiments, the disease or disorder is mediated by KRAS-G12D. In some embodiments, the disease or disorder is Juvenile Myelomonocytic Leukemia, Non-Small Cell Lung Carcinoma, Pancreatic Adenocarcinoma, Malignant Ovarian Neoplasm, Colorectal Carcinoma, Malignant Endometrial Neoplasm, Cholangiocarcinoma, Malignant Solid Tumor, Malignant Ovarian Epithelial Tumor, Esophageal Adenocarcinoma, Biliary Tract Carcinoma, Carcinoma, Colorectal Adenocarcinoma, Malignant Gastric Neoplasm, Malignant Colon Neoplasm, Neuroblastoma, Malignant Colorectal Neoplasm, Pancreatic Carcinoma, Intrahepatic Cholangiocarcinoma, Melanoma, Malignant Lung Neoplasm, Myelodysplastic/Myeloproliferative Neoplasm, Pancreatic Ductal Adenocarcinoma, Thyroid Gland Follicular Carcinoma, Malignant Pancreatic Neoplasm, or Lung Adenocarcinoma.

In some embodiments, the disease or disorder is associated with ALK. In some embodiments, the disease or disorder is mediated by ALK. In certain embodiments, the disease or disorder is Small Intestinal Carcinoma, Non-Small Cell Lung Carcinoma, Colon Carcinoma, Soft Tissue Sarcoma, Small Cell Lung Carcinoma, Astrocytoma, Rosai-Dorfman Disease, Colorectal Carcinoma, Malignant Thyroid Gland Neoplasm, Cholangiocarcinoma, Non-Squamous Non-Small Cell Lung Carcinoma, Glioblastoma, Malignant Solid Tumor, Acute Myeloid Leukemia, Leukemia, Erdheim-Chester Disease, Malignant Glioma, Hepatocellular Carcinoma, ALK-Positive Large B-Cell Lymphoma, B-Cell Non-Hodgkin Lymphoma, Thyroid Gland Medullary Carcinoma, EBV-Positive Diffuse Large B-Cell Lymphoma, Colorectal Adenocarcinoma, Systemic Anaplastic Large Cell Lymphoma, Thyroid Gland Undifferentiated (Anaplastic) Carcinoma, Malignant Colon Neoplasm, Hematopoietic and Lymphoid Malignancy, ALK-Positive Anaplastic Large Cell Lymphoma, Diffuse Large B-Cell Lymphoma, Malignant Pleural Mesothelioma, Squamous Cell Lung Carcinoma, Non-Hodgkin Lymphoma, Neuroblastoma, Ganglioneuroblastoma, Malignant Central Nervous System Neoplasm, Primary Cutaneous Anaplastic Large Cell Lymphoma, Malignant Colorectal Neoplasm, Low Grade Glioma, Gastric Carcinoma, Multiple Myeloma, Inflammatory Myofibroblastic Tumor, Pancreatic Carcinoma, Melanoma, Malignant Breast Neoplasm, Malignant Lung Neoplasm, Anaplastic Large Cell Lymphoma, Histiocytic and Dendritic Cell Neoplasm, Central Nervous System Neoplasm, Mature T-Cell and NK-Cell Lymphoma/Leukemia, Esophageal Carcinoma, Pancreatic Ductal Adenocarcinoma, Adenocarcinoma of the Gastroesophageal Junction, Langerhans Cell Histiocytosis, Lymphoma, Mature B-Cell Lymphoma/Leukemia, or Lung Adenocarcinoma.

In certain embodiments, the disease or disorder is associated with CCND-1. In certain embodiments, the disease or disorder is mediated by CCND-1. In some embodiments, the disease or disorder is Non-Small Cell Lung Carcinoma, Malignant Ovarian Neoplasm, Soft Tissue Sarcoma, Malignant Prostate Neoplasm, Oropharyngeal Squamous Cell Carcinoma, Nasopharyngeal Carcinoma, Mature B-Cell Lymphoma/Leukemia, Lung Carcinoma, Oral Cavity Carcinoma, Primary Central Nervous System Lymphoma, Laryngeal Squamous Cell Carcinoma, Malignant Solid Tumor, Osteosarcoma, Bronchogenic Carcinoma, AL Amyloidosis, Bladder Carcinoma, Malignant Glioma, B-Cell Non-Hodgkin Lymphoma, Hypopharyngeal Squamous Cell Carcinoma, Mantle Cell Lymphoma, Squamous Cell Lung Carcinoma, Non-Hodgkin Lymphoma, Malignant Colorectal Neoplasm, Multiple Myeloma, Melanoma, Malignant Breast Neoplasm, Acral Lentiginous Melanoma, Breast Carcinoma, Dedifferentiated Chondrosarcoma, Anaplastic Large Cell Lymphoma, Head and Neck Squamous Cell Carcinoma, Histiocytic and Dendritic Cell Neoplasm, Bladder Papillary Urothelial Neoplasm, Primary Malignant Liver Neoplasm, Chondrosarcoma, Lymphoma, Malignant Pancreatic Neoplasm, or Urothelial Carcinoma.

In some embodiments, the disease or disorder is associated with CCNE1. In some embodiments, the disease or disorder is mediated by CCNE1. In certain embodiments, the disease or disorder is Vaginal Carcinoma, High Grade Ovarian Serous Adenocarcinoma, Malignant Ovarian Neoplasm, Soft Tissue Sarcoma, Gastric Adenocarcinoma, Vulvar Carcinoma, Malignant Solid Tumor, Osteosarcoma, Hepatocellular Carcinoma, Penile Carcinoma, Anal Carcinoma, Synovial Sarcoma, Non-Hodgkin Lymphoma, Multiple Myeloma, Malignant Breast Neoplasm, Malignant Lung Neoplasm, Breast Carcinoma, Histiocytic and Dendritic Cell Neoplasm, Malignant Pancreatic Neoplasm, or Malignant Ovarian Epithelial Tumor.

In certain embodiments, the disease or disorder is BCL-2. In some embodiments, the disease or disorder is mediated by BCL-2. In some embodiments, the disease or disorder is Non-Small Cell Lung Carcinoma, Burkitt Lymphoma, Small Cell Lung Carcinoma, Acute Lymphoblastic Leukemia, Follicular Lymphoma, Malignant Endometrial Neoplasm, Invasive Breast Carcinoma, Glioblastoma, Malignant Solid Tumor, Acute Myeloid Leukemia, Chronic Lymphocytic Leukemia, B-Cell Non-Hodgkin Lymphoma, Diffuse Large B-Cell Lymphoma Activated B-Cell Type, EBV-Positive Diffuse Large B-Cell Lymphoma, High Grade B-Cell Lymphoma, High Grade B-Cell Lymphoma with MYC and BCL2 and/or BCL6 Rearrangements, Triple-Hit Lymphoma, Chronic Myeloid Leukemia, Transformed Non-Hodgkin Lymphoma, Double-Hit Lymphoma, B-Cell Acute Lymphoblastic Leukemia, Hematopoietic and Lymphoid Malignancy, Diffuse Large B-Cell Lymphoma, Non-Hodgkin Lymphoma, Multiple Myeloma, B-Cell Lymphoma, Hodgkin Lymphoma, Diffuse Large B-Cell Lymphoma, Breast Carcinoma, Head and Neck Squamous Cell Carcinoma, Germinal Center B-Cell-Like Diffuse Large B-Cell Lymphoma, Lymphoma, or Mature B-Cell Lymphoma/Leukemia.

In some embodiments, the disease or disorder is associated with MCL-1. In some embodiments, the disease or disorder is mediated by MCL-1. In some embodiments, the disease or disorder is Melanoma, Malignant Breast Neoplasm, Lymphoma, Non-Small Cell Lung Carcinoma, Malignant Ovarian Neoplasm, Breast Carcinoma, Acute Lymphoblastic Leukemia, Head and Neck Squamous Cell Carcinoma, Hepatocellular Carcinoma, Malignant Prostate Neoplasm, Malignant Thyroid Gland Neoplasm, Pancreatic Ductal Adenocarcinoma, Malignant Colorectal Neoplasm, Malignant Solid Tumor, or Multiple Myeloma.

In some embodiments, the disease or disorder is associated with MYC. In certain embodiments, the disease or disorder is mediated by MYC. In some embodiments, the disease or disorder is Vaginal Carcinoma, Pancreatic Adenocarcinoma, Soft Tissue Sarcoma, Marginal Zone Lymphoma, Small Cell Lung Carcinoma, Follicular Lymphoma, Medulloblastoma, B-Cell Non-Hodgkin Lymphoma, Ewing Sarcoma, Small Lymphocytic Leukemia, Peripheral T-Cell Lymphoma, Transformed Non-Hodgkin Lymphoma, Double-Hit Lymphoma, Intraocular Lymphoma, Neuroblastoma, Merkel Cell Carcinoma, Head and Neck Squamous Cell Carcinoma, Myeloid Neoplasm, Adenocarcinoma of the Gastroesophageal Junction, Hodgkin Lymphoma, Mature B-Cell Non-Hodgkin Lymphoma, High Grade Ovarian Serous Adenocarcinoma, Gastric Squamous Cell Carcinoma, Acute Lymphoblastic Leukemia, Vulvar Carcinoma, Malignant Prostate Neoplasm, Glioblastoma, Esophageal Adenocarcinoma, Small Intestinal Lymphoma, Breast Angiosarcoma, Esophageal Squamous Cell Carcinoma, Diffuse Large B-Cell Lymphoma Activated B-Cell Type, EBV-Positive Diffuse Large B-Cell Lymphoma, High Grade B-Cell Lymphoma, High Grade B-Cell Lymphoma with MYC and BCL2 and/or BCL6 Rearrangements, Triple-Hit Lymphoma, Diffuse Large B-Cell Lymphoma, Chronic Lymphocytic Leukemia/Small Lymphocytic Lymphoma, B-Cell Lymphoma, Pancreatic Carcinoma, Endometrial Serous Adenocarcinoma, Pancreatic Ductal Adenocarcinoma, Lymphoma, Burkitt Lymphoma, Malignant Solid Tumor, Acute Myeloid Leukemia, Osteosarcoma, Ovarian Carcinoma, Leukemia, Bladder Carcinoma, Chronic Lymphocytic Leukemia, Penile Carcinoma, Anal Carcinoma, Sarcoma, Hematopoietic and Lymphoid Malignancy, Small Lymphocytic Lymphoma, Non-Hodgkin Lymphoma, Malignant Esophageal Neoplasm, Multiple Myeloma, Melanoma, Diffuse Large B-Cell Lymphoma, Malignant Breast Neoplasm, Breast Carcinoma, Germinal Center B-Cell-Like Diffuse Large B-Cell Lymphoma, Esophageal Adenosquamous Carcinoma, Non-Small Cell Lung Carcinoma, Malignant Ovarian Neoplasm, Prostate Carcinoma, Gastric Adenocarcinoma, Colorectal Carcinoma, Hairy Cell Leukemia, Gastric Adenosquamous Carcinoma, Brain Glioblastoma, Head and Neck Carcinoma, Esophageal Adenoid Cystic Carcinoma, Mantle Cell Lymphoma, Malignant Colorectal Neoplasm, Lymphoplasmacytic Lymphoma, Malignant Lung Neoplasm, or Mature B-Cell Lymphoma/Leukemia.

In some embodiments, the disease or disorder is associated with beta-catenin. In certain embodiments, the disease or disorder is mediated by beta-catenin. In some embodiments, the disease or disorder is Desmoplastic/Nodular Medulloblastoma, Non-Small Cell Lung Carcinoma, Colon Carcinoma, Gastric Adenocarcinoma, Colorectal Carcinoma, Medulloblastoma, Malignant Endometrial Neoplasm, Malignant Solid Tumor, Acute Myeloid Leukemia, Hepatocellular Carcinoma, Desmoid-Type Fibromatosis, Large Cell/Anaplastic Medulloblastoma, Colorectal Adenocarcinoma, Malignant Colon Neoplasm, Medulloblastoma with Extensive Nodularity, Malignant Colorectal Neoplasm, Intracranial Primitive Neuroectodermal Neoplasm, Malignant Lung Neoplasm, Primary Malignant Liver Neoplasm, or Pancreatic Ductal Adenocarcinoma.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1: Synthesis of Intermediates to Compounds of the Present Disclosure

### Synthetic Procedure of Intermediates Int-1 and Int-2

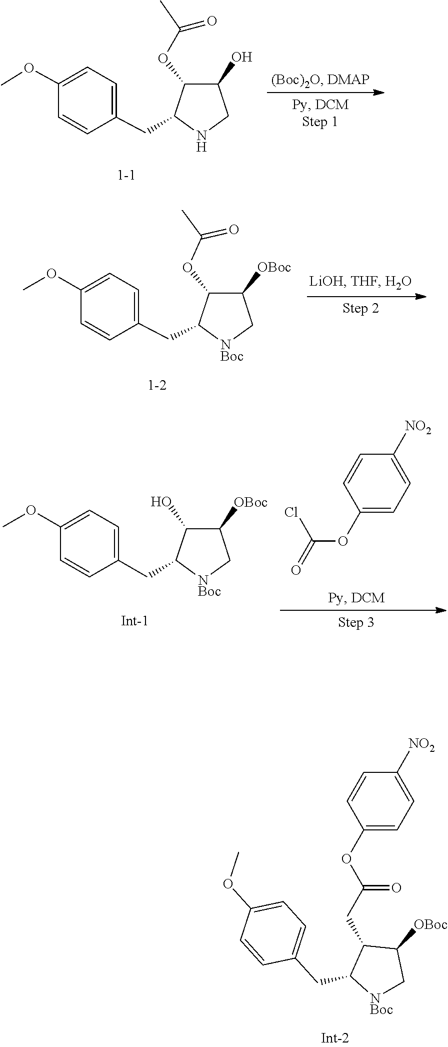

Step 1: To a stirred solution of anisomycin (5 g, 18.85 mmol, 1 eq.) in Pyridine (100 mL) was added Boc₂O (41.13 g, 188.46 mmol, 10 eq.) and DMAP (3.45 g, 28.27 mmol, 1.5 eq.) at room temperature. The resulting mixture was stirred over night at 25° C. Upon completion, the reaction mixture was concentrated in vacuo. The residue was purified by reversed-phase flash chromatography (0.05% NH₄HCO₃ in H₂O/ACN) to afford tert-butyl (2R,3S,4S)-3-(acetyloxy)-4-[(tert-butoxycarbonyl)oxy]-2-[(4-methoxyphenyl)methyl]pyrrolidine-1-carboxylate (8 g, 91.2%) as a light yellow oil. MS: m/z: Calc'd for C₂₄H₃₅NO₈ [M+H−56−100]⁺310. found 310.

Step 2: To a stirred solution of tert-butyl (2R,3S,4S)-3-(acetyloxy)-4-[(tert-butoxycarbonyl)oxy]-2-[(4-methoxyphenyl)methyl]pyrrolidine-1-carboxylate (8 g, 17.18 mmol, 1 eq.) in THF (100 mL) was added LiOH (1.23 g, 51.55 mmol, 3 eq.) and H₂O (10 mL) at room temperature. The resulting mixture was stirred over night at 25° C. The resulting mixture was extracted with EtOAc (3×30 mL). The combined organic layers were washed with water (3×10 mL), dried over anhydrous Na₂SO₄. After filtration, the filtrate was concentrated under reduced pressure to afford crude product tert-butyl (2R,3S,4S)-4-[(tert-butoxycarbonyl)oxy]-3-hydroxy-2-[(4-methoxyphenyl)methyl]pyrrolidine-1-carboxylate (7.2 g, 98.9%) as a light yellow solid, which was used directly in the next step without further purification. MS: m/z: Calc'd for C₂₂H₃₃NO₇ [M+H−56−56]⁺312. found 312.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (4.7 g, 11.10 mmol, 1 eq.) and 4-nitrophenyl carbonochloridate (3.36 g, 16.67 mmol, 1.5 eq.) in DCM (50 mL) was added Pyridine (1.76 g, 22.25 mmol, 2 eq.) at room temperature. The resulting mixture was stirred at room temperature for another 2 h. After completion of reaction monitored by LCMS. The mixture was concentrated, the residue was purified by silica gel column chromatography, eluted with PE/EA (5:1) to afford tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (5.4 g, 82.7%) as a light yellow oil. MS: m/z: Calc'd for C₂₉H₃₆N₂O₁₁ [M+H−100]⁺489. found 489.

### Synthetic Procedure of Intermediates Int-3 and Int-4

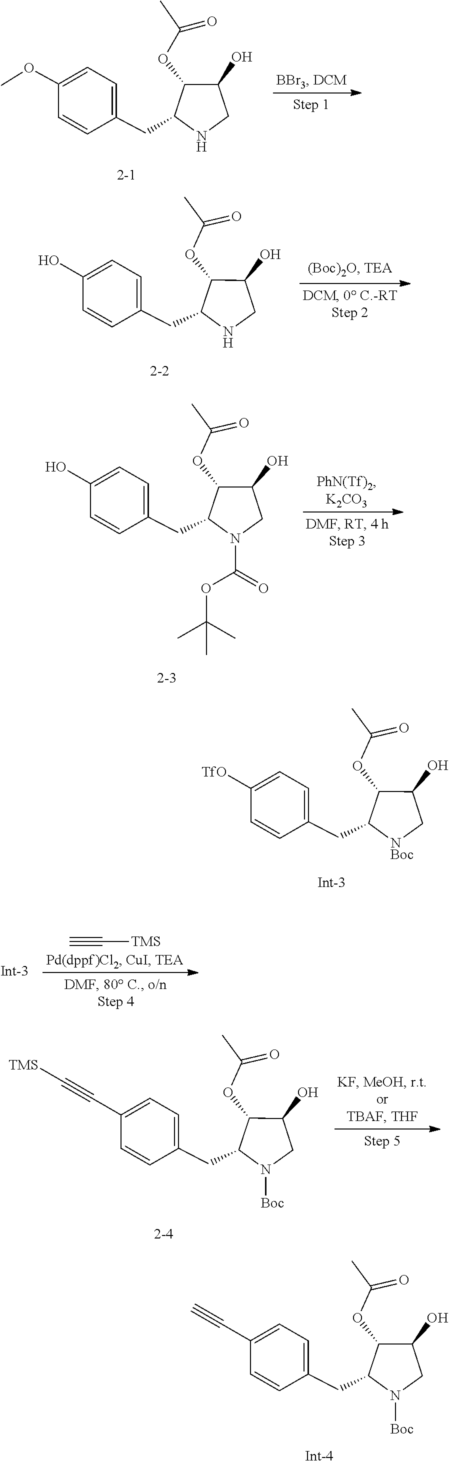

Step 1: To a stirred solution of anisomycin (1.5 g, 5.65 mmol, 1 equiv) in Dichloromethane (8 mL) was added Boron tribromide (16.8 mL, 3 equiv) at −78° C. The reaction mixture was stirred at −78° C. for 2 h and warmed to room temperature. The mixture was stirred for 1 h at room temperature and quenched by saturated NaHCO₃ solution. The DCM was removed, and the solution was lyophilized to obtain (2R,3S,4S)-4-hydroxy-2-[(4-hydroxyphenyl)methyl]pyrrolidin-3-yl acetate (1.8 g, 85.41%) as a crude. MS: m/z: Calc'd for C₁₃H₁₇NO₄ [M+H]⁺ 252. found 252.

Step 2: To a stirred solution of (2R,3S,4S)-4-hydroxy-2-[(4-hydroxyphenyl)methyl]pyrrolidin-3-yl acetate (1.8 g, 7.16 mmol, 1 equiv) and triethylamine (2.54 g, 25.07 mmol, 3.5 equiv) in DCM (30 mL) was added di-tert-butyl dicarbonate (1.88 g, 8.59 mmol, 1.2 equiv) at 0° C. The mixture was stirred at room temperature for 3 h. After completion of the reaction monitored by LCMS, the reaction mixture was filtrated. The filtrate was concentrated. The residue was purified by a reversed-phase column to obtain tert-butyl (2R,3S,4S)-3-(acetyloxy)-4-hydroxy-2-[(4-hydroxyphenyl)methyl]pyrrolidine-1-carboxylate (1.7 g, 67.54%) as a white solid. MS: m/z: Calc'd for C₁₈H₂₅NO₆ [M−H]⁻ 350. found 350.

Step 3: To a stirred solution of tert-butyl (2R,3S,4S)-3-(acetyloxy)-4-hydroxy-2-[(4-hydroxyphenyl)methyl]pyrrolidine-1-carboxylate (1.7 g, 4.83 mmol,) and Potassium carbonate (2.01 g, 14.51 mmol, 3 equiv) in DMF (16 mL) was added 1,1,1-trifluoro-N-phenyl-N-trifluoromethanesulfonylmethanesulfonamide (2.25 g, 6.28 mmol, 1.3 equiv) at 0° C. The reaction mixture was stirred at room temperature for 2 h. After completion of the reaction monitored by LCMS, the reaction mixture was filtrated. The filtrate was injected into a reversed-phase column and purified to obtain tert-butyl (2R,3S,4S)-3-(acetyloxy)-4-hydroxy-2-{[4-(trifluoromethanesulfonyloxy)phenyl]methyl}pyrrolidine-1-carboxylate (1.5 g, 64.13%) as a white solid. MS: m/z: Calc'd for C₁₉H₂₄F₃NO₈S [M+NH₄]⁺501. found 501.

Step 4: To a stirred solution of tert-butyl (2R,3S,4S)-3-(acetyloxy)-4-hydroxy-2-{[4-(trifluoromethanesulfonyloxy)phenyl]methyl}pyrrolidine-1-carboxylate (200 mg, 0.42 mmol, 1 equiv) and trimethylsilylacetylene (121.9 mg, 1.24 mmol, 3 equiv) in DMF (10 mL) was added TEA (167.4 mg, 1.65 mmol, 4 equiv), CuI (7.9 mg, 0.04 mmol, 0.1 equiv) and Pd(dppf)Cl₂·CH₂Cl₂ (50.6 mg, 0.06 mmol, 0.15 equiv) in portions at room. The reaction was placed under vacuum, sonicated and backfilled with nitrogen. The solution was stirred at 80° C. for 12 h. Desired product could be detected by LCMS. Water was used to quench the reaction, extracted with EA, concentrated, the residue was purified by Prep-TLC (PE/EA 2:1) to afford tert-butyl (2R,3S,4S)-3-(acetyloxy)-4-hydroxy-2-({4-[2-(trimethylsilyl)ethynyl] phenyl}methyl)pyrrolidine-1-carboxylate (160 mg, 89.6%) as a yellow oil. MS: m/z: Calc'd for C₂₃H₃₃NO₅Si [M-100]⁺ 332. found 332.

Step 5: A solution of tert-butyl (2R,3S,4S)-3-(acetyloxy)-4-hydroxy-2-({4-[2-(trimethylsilyl) ethynyl]phenyl}methyl)pyrrolidine-1-carboxylate (170 mg, 0.39 mmol, 1 equiv) and Triethylamine trihydrofluoride (190.5 mg, 1.18 mmol, 3.0 equiv) in THF (5 mL) was stirred at 60° C. for 12 h. Desired product could be detected by LCMS. Concentrated, the residue was purified by Prep-TLC (PE/EA 3:1) to afford tert-butyl (2R,3S,4S)-3-(acetyloxy)-2-[(4-ethynylphenyl)methyl]-4-hydroxypyrrolidine-1-carboxylate (135 mg, 95.36%) as a light yellow solid. MS: m/z: Calc'd for C₂₀H₂₅NO₅ [M-56]⁺304. found 304.

### Synthetic Procedure of Intermediate Int-5A

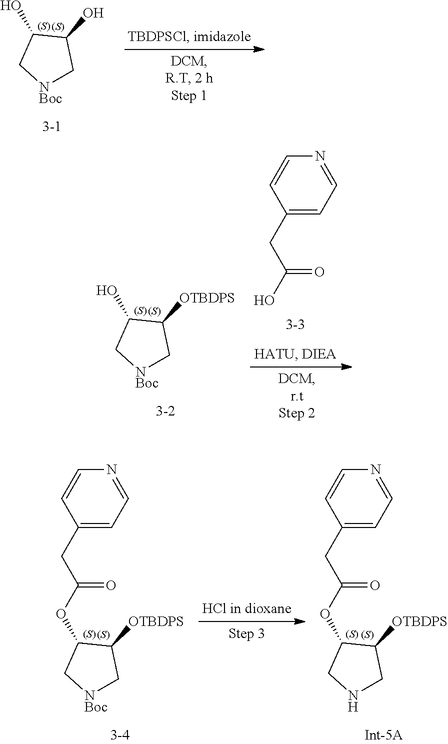

Step 1: To a stirred solution of tert-butyl (3S,4S)-3,4-dihydroxypyrrolidine-1-carboxylate (3-1, 1 g, 4.92 mmol, 1 eq.), Imidazole (1 g, 14.76 mmol, 3 eq.) and TBDPSCl (1.2 g, 4.43 mmol, 0.9 eq.) in DCM (10 mL) was stirred for 1 h at room temperature. Desired product could be detected by LCMS. Concentrated, the residue was purified by silica gel column chromatography, eluted with PE/EA (2:1) to afford tert-butyl (3S,4S)-3-[(tert-butyldiphenylsilyl)oxy]-4-hydroxypyrrolidine-1-carboxylate (1.3 g, 59.8%) as a colorless oil. MS: m/z: Calc'd for C₂₅H₃₅NO₄Si [M−H]⁻ 440. found 440.

Step 2: To a solution of tert-butyl (3S,4S)-3-[(tert-butyldiphenylsilyl)oxy]-4-hydroxypyrrolidine-1-carboxylate (3-2, 1.3 g, 2.94 mmol, 1 eq.) in DCM (15 mL) was added 4-acetylpyridine (3-3, 0.6 g, 4.42 mmol, 1.5 eq.), DCC (0.9 g, 4.42 mmol, 1.5 eq.) and DMAP (360 mg, 2.94 mmol, 1 eq.). The mixture was stirred at room temperature for 1 h. Desired product could be detected by LCMS. The resulting mixture was filtered, the filter cake was washed with ethyl acetate (3×5 mL). The filtrate was concentrated under reduced pressure. The residue was purified by silica gel column chromatography, eluted with PE/EA (1:1) to afford tert-butyl (3S,4S)-3-[(tert-butyldiphenylsilyl)oxy]-4-{[2-(pyridin-4-yl)acetyl]oxy}pyrrolidine-1-carboxylate (1.6 g, 96.9%) as a colorless oil. MS: m/z: Calc'd for C₃₂H₄₀N₂O₅Si [M-56]⁺505. found 505.

Step 3: To a solution of tert-butyl (3S,4S)-3-[(tert-butyldiphenylsilyl)oxy]-4-{[2-(pyridin-4-yl)acetyl]oxy}pyrrolidine-1-carboxylate (1.6 g, 2.85 mmol) in dioxane (15 mL) was added HCl(gas) in 1,4-dioxane (15 mL). The mixture was stirred at room temperature for 1 h. Desired product could be detected by LCMS. The mixture was concentrated under reduced pressure to afford crude product which was used in the next step directly without further purification. MS: m/z: Calc'd for C₂₇H₃₂N₂O₃Si [M+H]⁺461. found 461.

### Example 2: Synthesis of Compounds of the Present Disclosure

General Procedure I: Condensation Reaction with DCC and DMAP

To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int-1, 0.24 mmol, 1 eq.) and corresponding acid (2 eq.) in DCM (5 mL) was added DCC (1.5 eq.) and DMAP (1 eq.). The reaction mixture was stirred at ambient temperature for 2 h. Upon completion, the reaction mixture was quenched with water and extracted with EA. The extracts were washed with brine, dried over anhydrous sodium sulfate, filtered and concentrated. The residue was purified by reversed column chromatography.

### General Procedure II: Substitution Reaction for Synthesis of Carbamate

To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2, 0.12 mmol, 1 eq.) and corresponding amine (1 eq.) in ACN (5 mL) was added DIEA (3 eq.) at room temperature. The resulting mixture was stirred at room temperature for 12 h. After completion, the reaction mixture was concentrated in vacuo. The residue was purified by reversed column chromatography.

### General Procedure III: Boc Deprotection

The corresponding Boc-protected amine (1 equiv) was dissolved in anhydrous CH₂Cl₂ (5 mL/mmol), and TFA (5 mL/mmol) was added. The mixture was stirred at r.t. for 2 h. After removal of the volatiles, the oily residue was further dried under high vacuum. The residue was purified by prep-HPLC.

General Procedure IV: Reductive Amination with AcOH and NaBH(OAc)₃

To a stirred solution of (3S,4S)-4-[(tert-butyldiphenylsilyl)oxy]pyrrolidin-3-yl 2-(pyridin-4-yl)acetate (Int-5A, 0.16 mmol, 1 eq.) and corresponding benzaldehyde (1.5 eq.) in DCM (2 mL) was added DIEA (2 eq.) and AcOH (2 eq.), the resulting mixture was stirred at room temperature for 1 h. Then NaBH(OAc)₃ (2 eq.) was added at 0° C. and the resulting mixture was stirred at RT for 1 h. Upon completion, the mixture was cooled to r.t., concentrated in vacuo. The residue was purified by reversed-phase flash chromatography.

### General Procedure V: TBDPS Deprotection

The corresponding TBDPS-protected alcohol (1 equiv) was dissolved in anhydrous THF (5 mL/mmol), and TBAF (1 equiv) was added at 0° C. The mixture was stirred at r.t. for 2 h. Upon completion, the mixture was concentrated in vacuo. The residue was purified by prep-HPLC.

### General Procedure VI: Cyclization Reaction for Synthesis of Substituted Triazoles

To a stirred solution of tert-butyl (2R,3S,4S)-4-[(tert-butoxycarbonyl)oxy]-2-[(4-methoxyphenyl)methyl]-3-(prop-2-yn-1-yloxy)pyrrolidine-1-carboxylate (Int-4, 80 mg, 0.17 mmol, 1 eq.) and corresponding azide (1 eq.) in MeOH (4 mL) was added CuSO₄·5H₂O (1 eq.) and sodium (5R)-5-[(1S)-1,2-dihydroxyethyl]-3,4-dihydroxy-2,5-dihydrofuran-2-one (2 eq.) at 0° C. The resulting mixture was stirred at room temperature for overnight. Desired product could be detected by LCMS. The resulting mixture was concentrated under reduced pressure. The residue was purified by reversed-phase flash chromatography.

### Synthetic Procedure of V187189

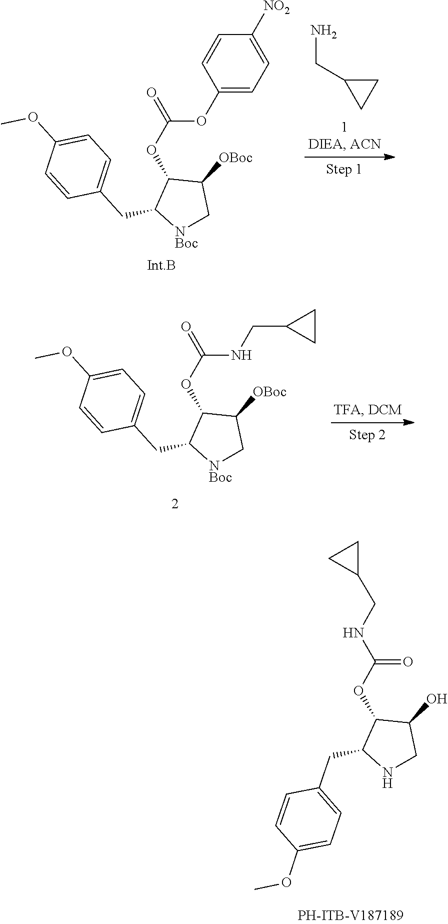

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1.0 eq.) and 1-cyclopropylmethanamine (24.2 mg, 0.34 mmol, 2.0 eq.) in MeCN (3 mL) was added DIEA (65.87 mg, 0.51 mmol, 3.0 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl) oxy]-3-[(cyclopropylmethylcarbamoyl)oxy]-2-[(4-methoxyphenyl)methyl]pyrrolidine-1-carboxylate (80 mg, 90.45%) as a yellow oil. MS: m/z: Calc'd for C₂₇H₄₀N₂O₈ [M+H−56−100]⁺365. Found, 365.

Step 2: To a stirred solution of tert-butyl (2R,3S,4S)-4-[(tert-butoxycarbonyl)oxy]-3-[(cyclopropylmethylcarbamoyl)oxy]-2-[(4-methoxyphenyl)methyl]pyrrolidine-1-carboxylate (55 mg, 0.11 mmol, 1.0 eq.) in DCM (3 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-(cyclopropylmethyl)carbamate; trifluoroacetic acid (21 mg, 45.7%) as a white solid. MS: m/z: Calc'd for C₁₇H₂₄N₂O₄ [M+H]⁺321. Found, 321. ¹H NMR (400 MHz, Methanol-d₄) δ 7.28-7.20 (m, 2H), 6.97-6.89 (m, 2H), 4.94 (d, J=3.5 Hz, 1H), 4.38 (d, J=4.3 Hz, 1H), 4.17-4.13 (m, 1H), 3.80 (s, 3H), 3.58 (dd, J=12.6, 4.3 Hz, 1H), 3.22 (d, J=12.6 Hz, 1H), 3.17-2.93 (m, 4H), 1.08-1.00 (m, 1H), 0.59-0.50 (m, 2H), 0.28-0.24 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 5% B to 55% B in 10 min; Wave Length: 254 nm/220 nm; RT1(min): 8.68

### Synthetic Procedure of V187190

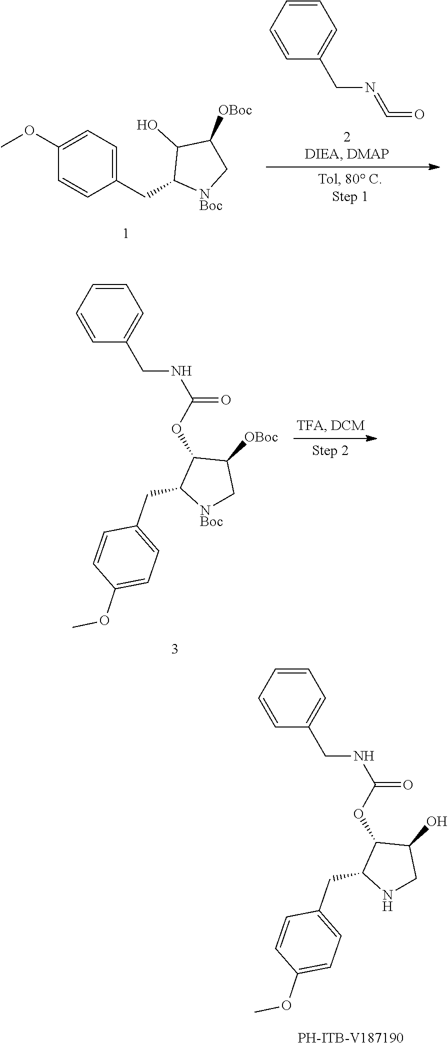

Step 1: To a stirred solution of tert-butyl (2R,4S)-4-[(tert-butoxycarbonyl)oxy]-3-hydroxy-2-[(4-methoxyphenyl)methyl]pyrrolidine-1-carboxylate (Int A, 80 mg, 0.19 mmol, 1 eq.) and (isocyanatomethyl)benzene (50.3 mg, 0.38 mmol, 2 eq.) in Toluene (8 mL) were added DIEA (73.2 mg, 0.57 mmol, 3 eq.) and DMAP (23.1 mg, 0.19 mmol, 1 eq.) and the resulting mixture was stirred at 80° C. for overnight. The mixture was concentrated, the crude was purified by column chromatography to obtain tert-butyl (2R,3S,4S)-3-[(benzylcarbamoyl)oxy]-4-[(tert-butoxycarbonyl)oxy]-2-[(4-methoxyphenyl)methyl]pyrrolidine-1-carboxylate (85 mg, 80.8%) as a yellow oil. MS: m/z: Calc'd for C₃₀H₄₀N₂O₈ [M+H−100−56]⁺401. found 401.

Step 2: To a stirred solution of tert-butyl (2R,3S,4S)-3-[(benzylcarbamoyl)oxy]-4-[(tert-butoxycarbonyl)oxy]-2-[(4-methoxyphenyl)methyl]pyrrolidine-1-carboxylate (60 mg, 0.11 mmol, 1 eq.) in DCM (5 mL) was added TFA (1 mL) and the resulting mixture was stirred at room temperature for 2 h. After completion of the reaction monitored by LCMS, then the mixture was concentrated and the residue was purified by Prep-HPLC to obtain (2R,3S,4S)-4-hydroxy-2-[(4-methoxyphenyl)methyl]pyrrolidin-3-yl N-benzylcarbamate; trifluoroacetic acid (25.3 mg, 49.1%) as a white solid. MS: m/z: Calc'd for C₂₀H₂₄N₂O₄ [M+H]⁺357. found 357. ¹H NMR (400 MHz, Methanol-d₄) δ 7.41-7.26 (m, 5H), 7.20 (d, J=8.2 Hz, 2H), 6.87 (d, J=8.1 Hz, 2H), 4.93 (d, J=3.3 Hz, 1H), 4.43-4.35 (m, 2H), 4.30 (d, J=15.1 Hz, 1H), 4.16 (dd, J=7.9, 3.4 Hz, 1H), 3.79 (s, 3H), 3.59 (dd, J=13.7, 3.5 Hz, 1H), 3.23 (d, J=12.6 Hz, 1H), 3.10 (dd, J=14.0, 7.7 Hz, 1H), 2.98 (dd, J=14.2, 8.5 Hz, 1H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm; RT1(min): 8.5

### Synthetic Procedure of V187191

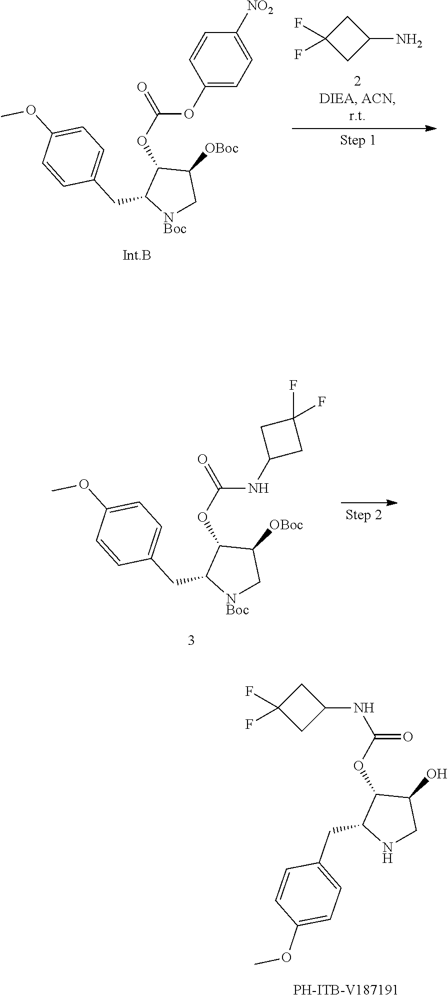

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int B, 100 mg, 0.17 mmol, 1 eq.) and 3,3-difluorocyclobutan-1-amine (18.2 mg, 0.17 mmol, 1 eq.) in ACN (8 mL) was added DIEA (65.8 mg, 0.51 mmol, 3 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl)oxy]-3-{[(3,3-difluorocyclobutyl)carbamoyl]oxy}-2-[(4-methoxyphenyl)methyl]pyrrolidine-1-carboxylate (60 mg, 63.4%) as a white solid. MS: m/z: Calc'd for C₂₇H₃₈F₂N₂O₈ [M+H−100−56]⁺401. found 401.

Step 2: To a stirred solution of tert-butyl (2R,3S,4S)-4-[(tert-butoxycarbonyl)oxy]-3-{[(3,3-difluorocyclobutyl)carbamoyl]oxy}-2-[(4-methoxyphenyl)methyl]pyrrolidine-1-carboxylate (55 mg, 0.10 mmol, 1 eq.) in DCM (5 mL) was added TFA (1 mL) at room temperature and the resulting mixture was stirred for 2 h. Then the mixture was concentrated and purified by Prep-HPLC to obtain (2R,3S,4S)-4-hydroxy-2-[(4-methoxyphenyl)methyl]pyrrolidin-3-yl N-(3,3-difluorocyclobutyl)carbamate; trifluoroacetic acid (28.6 mg, 61.4%) as a white solid. MS: m/z: Calc'd for C₁₇H₂₂F₂N₂O₄[M+H]⁺357. found 357. ¹H NMR (400 MHz, Methanol-d₄) δ 7.23 (d, J=8.3 Hz, 2H), 6.93 (d, J=8.2 Hz, 2H), 4.95 (d, J=3.5 Hz, 1H), 4.39 (d, J=4.2 Hz, 1H), 4.18 (t, J=6.0, 4.3 Hz, 1H), 4.01 (d, J=7.3 Hz, 1H), 3.80 (s, 3H), 3.57 (dd, J=12.6, 4.3 Hz, 1H), 3.22 (d, J=12.7 Hz, 1H), 3.11 (dd, J=14.1, 7.2 Hz, 1H), 3.02-2.95 (m, 3H), 2.66-2.51 (m, 2H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min m/min; Gradient: 2% B to 30% B in 10 min; Wave Length: 254 nm/220 nm; RT1(min): 8.5

### Synthetic Procedure of V187192

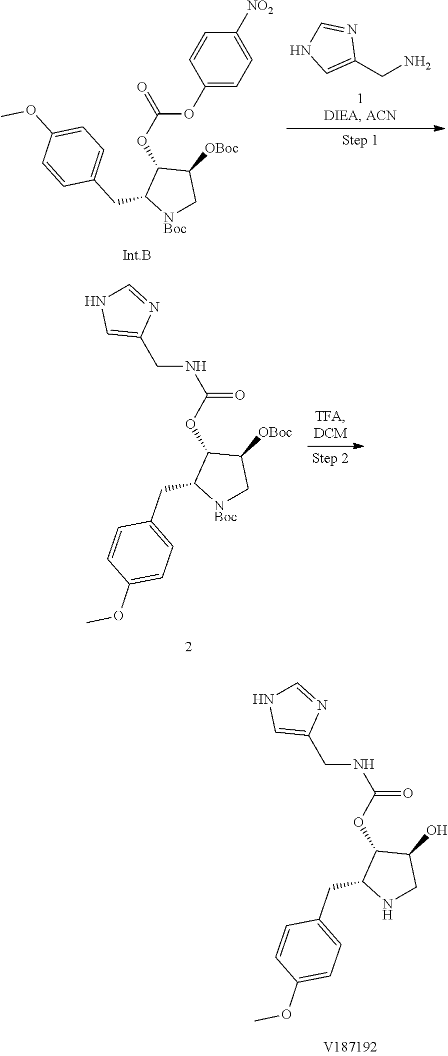

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq.) and 1-(1H-imidazol-4-yl)methanamine (33 mg, 0.34 mmol, 2 eq.) in MeCN (3 mL) was added DIEA (88 mg, 0.68 mmol, 4 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl) oxy]-3-{[(1H-imidazol-4-ylmethyl)carbamoyl]oxy}-2-[(4-methoxyphenyl)methyl]pyrrolidine-1-carboxylate (60 mg, 32.3%) as a white solid. MS: m/z: Calc'd for C₂₇H₃₈N₄O₈ [M+H]⁺547. Found, 547.

Step 2: To a stirred solution of tert-butyl (2R,3S,4S)-4-[(tert-butoxycarbonyl)oxy]-3-{[(1H-imidazol-4-ylmethyl)carbamoyl]oxy}-2-[(4-methoxyphenyl)methyl]pyrrolidine-1-carboxylate (60 mg, 0.11 mmol) in DCM (3 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-(1H-imidazol-4-ylmethyl)carbamate (12.5 mg, 19.6%) as a white solid. MS: m/z: Calc'd for C₁₇H₂₂N₄O₄ [M+H]⁺347. Found, 347. ¹H NMR (400 MHz, Methanol-d₄) δ 7.66 (s, 1H), 7.12 (d, J=8.3 Hz, 2H), 7.02 (s, 1H), 6.82 (d, J=8.4 Hz, 2H), 4.66 (s, 1H), 4.36-4.23 (m, 2H), 4.18 (s, 1H), 3.77 (s, 3H), 3.56 (s, 1H), 2.97-2.63 (m, 4H). Prep-HPLC-conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min mL/min; Gradient: 2% B to 32% B in 13 min; Wave Length: 254 nm/220 nm; RT1(min): 4.67.

### Synthetic Procedure of V187315

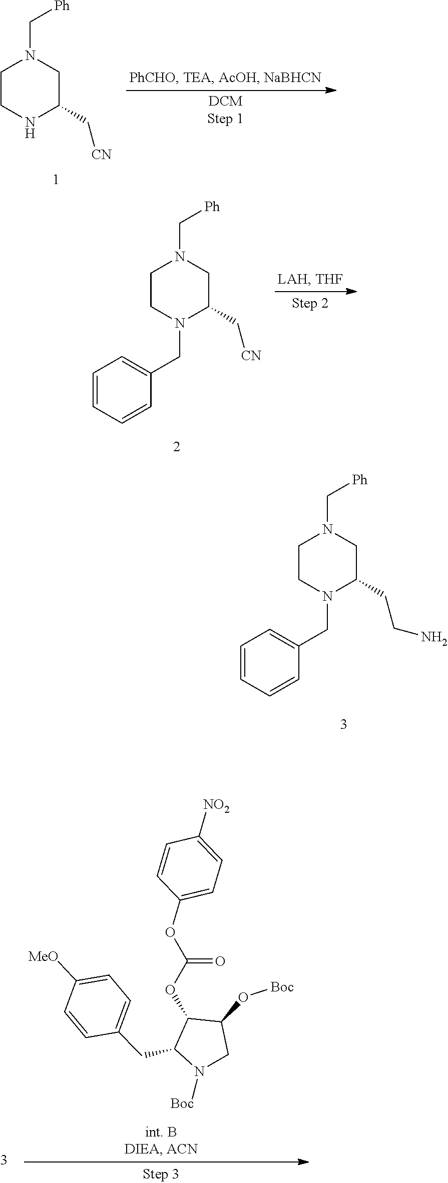 [SMILES: N#CC[C@H]1CN(Cc2ccccc2)CCN1 ; N#CC[C@H]1CN(Cc2ccccc2)CCN1Cc1ccccc1 ; NCC[C@H]1CN(Cc2ccccc2)CCN1Cc1ccccc1 ; COc1ccc(C[C@@H]2[C@H](OC(=O)Oc3ccc([N+](=O)[O-])cc3)[C@@H](OC(=O)OC(C)(C)C)CN2C(=O)OC(C)(C)C)cc1]

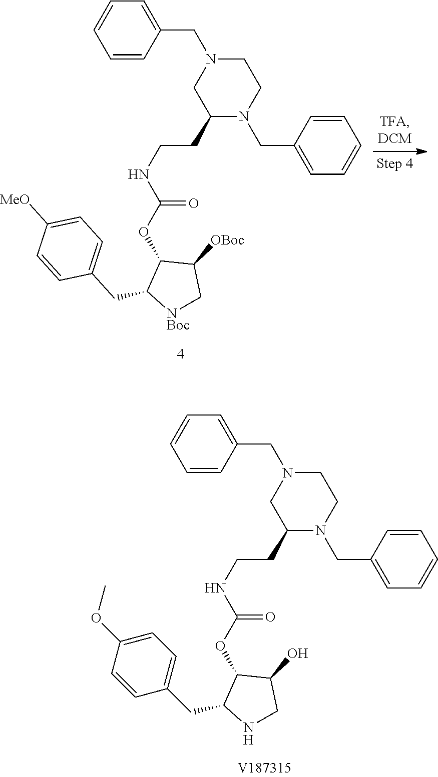

Step 1: To a stirred solution of 2-[(2S)-4-benzylpiperazin-2-yl]acetonitrile (400 mg, 1.86 mmol, 1 eq.) and benzaldehyde (296 mg, 2.79 mmol, 1.5 equiv) in DCM (5 ml) were added TEA (188 mg, 1.86 mmol, 1 eq.) and AcOH (223 mg, 3.72 mmol, 2 eq.) dropwise at room temperature. The reaction mixture was stirred for 30 min. Then NaBH(OAc)₃ (2 eq.) was added at 0° C. and the resulting mixture was stirred at RT for 2h. Upon completion, the mixture was concentrated under vacuo. The resulting residue was dissolved with DMSO and purified by a reversed-phase column chromatography (0.05% TFA in water and acetonitrile) to afford 2-[(2S)-1,4-dibenzylpiperazin-2-yl]acetonitrile (100 mg, 15.7%) as a white solid. MS: m/z: Calc'd for C₂₀H₂₃N₃[M+H]⁺306. found 306.

Step 2: To a stirred solution of 2-[(2S)-1,4-dibenzylpiperazin-2-yl]acetonitrile (100 mg, 0.33 mmol, 1 equiv) in THF (5 ml) was added LAH (4 M, 0.3 mL) dropwise at 0° C. The final reaction mixture was stirred for 2 h. Upon completion, the reaction mixture was quenched with ice water (0.5 ml) and directly purified by reversed phase column chromatography to afford product 2-[(2S)-1,4-dibenzylpiperazin-2-yl]ethanamine (76 mg, 45.0%) as a white solid. MS: m/z: Calc'd for C₂₀H₂₇N₃ [M+H]⁺310. Found 310.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 76 mg, 0.13 mmol, 1 eq.) and 2-[(2S)-1,4-dibenzylpiperazin-2-yl]ethanamine (80 mg, 0.26 mmol, 2 eq.) in MeCN (3 mL) was added DIEA (67 mg, 0.52 mmol, 4 eq.). The resulting reaction mixture was stirred at ambient temperature for overnight. After completion of the reaction monitored by LCMS, the mixture was purified directly by reversed-phase column chromatography (0.05% TFA in water and acetonitrile) to afford tert-butyl (2R,3S,4S)-4-[(tert-butoxycarbonyl)oxy]-3-[((2-[(2S)-1,4-dibenzylpiperazin-2-yl]ethylcarbamoyl)oxy]-2-[(4-methoxyphenyl)methyl]pyrrolidine-1-carboxylate (70 mg, 71.4%) as a yellow solid. MS: m/z: Calc'd for C₄₃H₅₈N₄O₈ [M+H]⁺759. Found, 759.

Step 4: To a stirred solution of tert-butyl (2R,3S,4S)-4-[(tert-butoxycarbonyl)oxy]-3-[((2-[(2S)-1,4-dibenzylpiperazin-2-yl]ethylcarbamoyl)oxy]-2-[(4-methoxyphenyl)methyl]pyrrolidine-1-carboxylate (100 mg, 0.13 mmol) in DCM (5 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-(2-[(2S)-1,4-dibenzylpiperazin-2-yl]ethylcarbamate; trifluoroacetic acid (24.9 mg, 27.5%) as a white solid. MS: m/z: Calc'd for C₃₃H₄₂N₄O₄ [M+H]⁺559. Found, 559. ¹H NMR (400 MHz, Methanol-d₄) δ 7.49-7.32 (m, 10H), 7.27-7.19 (m, 2H), 6.95-6.87 (m, 2H), 4.96 (d, J=3.5 Hz, 1H), 4.55-4.21 (m, 2H), 4.21-3.92 (m, 3H), 3.78 (s, 3H), 3.71 (s, 1H), 3.61-3.46 (m, 1H), 3.32 (d, J=1.6 Hz, 4H), 3.21-3.03 (m, 4H), 2.97 (t, J=11.6 Hz, 3H), 2.75 (s, 1H), 2.05-2.03 (m, 2H). Prep-HPLC conditions: Column: Xselect CSH Prep C18 OBD Column, 19*250 mm, 5 μm; Mobile Phase A: Water (0.05% TFA), Mobile Phase B: ACN; Flow rate: 25 mL/min mL/min; Gradient: 14% B to 30% B in 10 min; Wave Length: 254 nm/220 nm; RT1(min): 9.6

### Synthetic Procedure of V187321

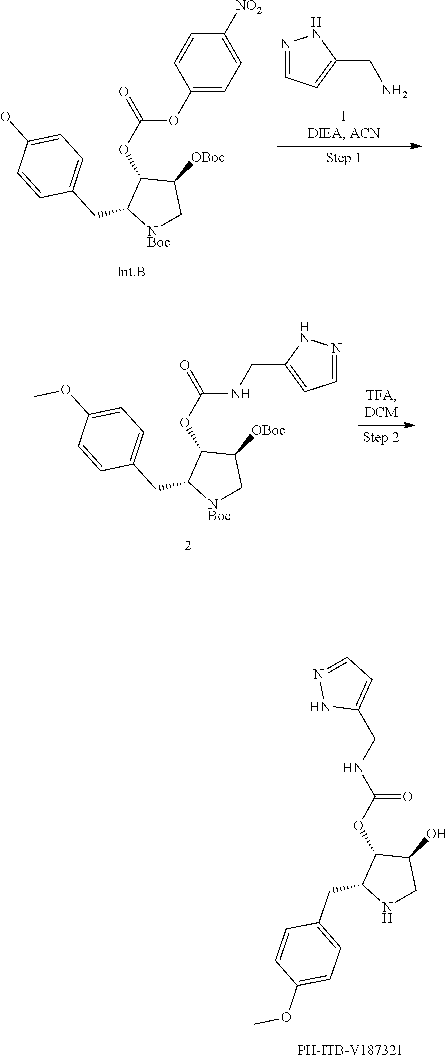

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1.0 eq.) and 3-methyl-2H-pyrazole; nitrogen (37.4 mg, 0.34 mmol, 2.0 eq.) in MeCN (3 mL) was added DIEA (88 mg, 0.68 mmol, 4 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl) oxy]-2-[(4-methoxyphenyl)methyl]-3-{[(2H-pyrazol-3-ylmethyl)carbamoyl]oxy}pyrrolidine-1-carboxylate (65 mg, 70.0%) as a light yellow oil. MS: m/z: Calc'd for C₂₇H₃₈N₄O₈ [M+H−56−56]⁺435. Found, 435.

Step 2: To a stirred solution of tert-butyl (2R,3S,4S)-4-[(tert-butoxycarbonyl)oxy]-2-[(4-methoxyphenyl)methyl]-3-{[(2H-pyrazol-3-ylmethyl)carbamoyl]oxy}pyrrolidine-1-carboxylate (60 mg, 0.11 mmol) in DCM (2 mL) was added TFA (1 mL) at room temperature. The resulting mixture was stirred at 25° C. for 1 h. The resulting mixture was concentrated under reduced pressure. The crude product (60 mg) was purified by Prep-HPLC to afford (2R,3S,4S)-4-hydroxy-2-[(4-methoxyphenyl)methyl]pyrrolidin-3-yl N-(2H-pyrazol-3-ylmethyl)carbamate; trifluoroacetic acid (16.3 mg, 31.74%) as a light yellow solid. MS: m/z: Calc'd for C₁₇H₂₂N₄O₄ [M+H]⁺347. Found, 347. ¹H NMR (400 MHz, Methanol-d₄) δ 7.66 (d, J=16.3 Hz, 1H), 7.21 (d, J=8.1 Hz, 2H), 6.90 (d, J=8.1 Hz, 2H), 6.35-6.33 (d, J=20.2 Hz, 1H), 4.95 (d, J=3.4 Hz, 1H), 4.47-4.27 (m, 3H), 4.18-4.14 (m, 1H), 3.64-3.50 (m, 1H), 3.33 (s, 3H), 3.22 (d, J=12.5 Hz, 1H), 3.11 (dd, J=14.1, 7.4 Hz, 1H), 2.97 (dd, J=13.9, 9.2 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 24% B in 10 min; Wave Length: 254 nm/220 nm; RT1(min): 8.68

### Synthetic Procedure of V187327

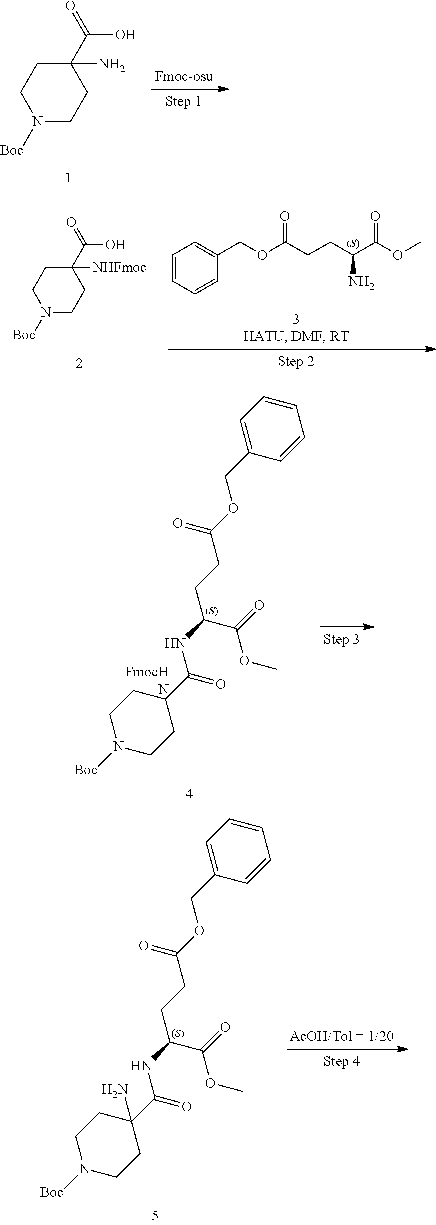

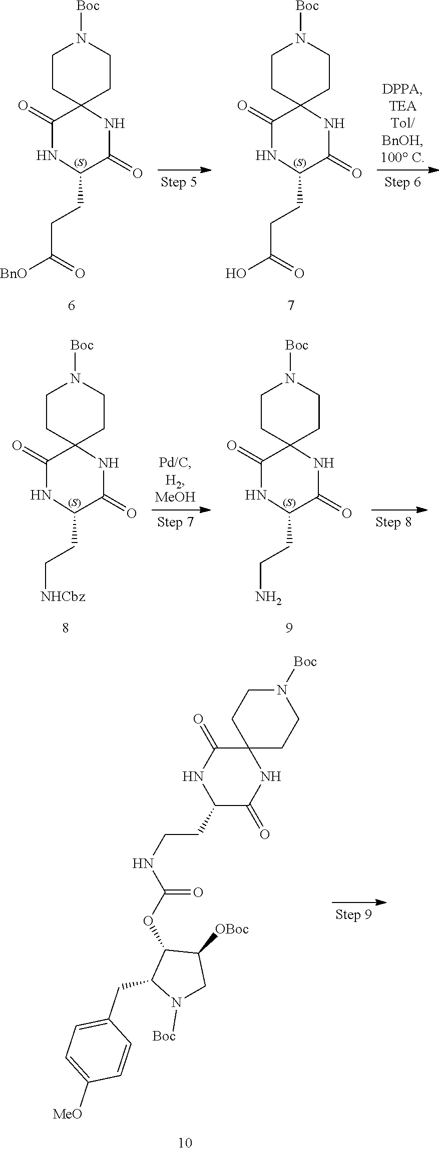

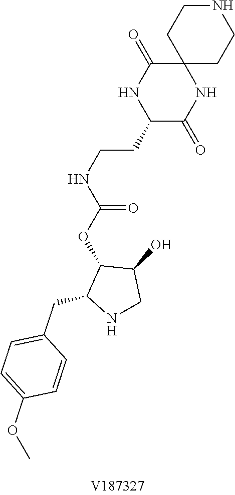 [SMILES: COc1ccc(C[C@H]2NC[C@H](O)[C@H]2OC(=O)NCC[C@@H]2NC(=O)C3(CCNCC3)NC2=O)cc1]

Step 1: To a stirred solution of 4-amino-1-(tert-butoxycarbonyl)piperidine-4-carboxylic acid (2000 mg, 8.18 mmol, 1 eq.) and 2,5-dioxopyrrolidin-1-yl 9H-fluoren-9-ylmethyl carbonate (6904.2 mg, 20.46 mmol, 2.5 eq.) in THF (20 mL) and water (20 mL) was added Na₂CO₃ (3470.8 mg, 32.74 mmol, 4 eq.) and the resulting mixture was stirred at room temperature for 2v h. The reaction mixture was quenched with water and extracted with EA. The extracts were washed with brine, dried over anhydrous sodium sulfate, filtered and concentrated. The residue was purified by flash to obtain 1-(tert-butoxycarbonyl)-4-{[(9H-fluoren-9-ylmethoxy)carbonyl]amino}piperidine-4-carboxylic acid (3000 mg, 78.5%) as a white solid. MS: m/z: Calc'd for C₂₆H₃₀N₂O₆ [M+H−100]⁺367. found 367.

Step 2: To a stirred solution of 1-(tert-butoxycarbonyl)-4-{[(9H-fluoren-9-ylmethoxy)carbonyl]amino}piperidine-4-carboxylic acid (2000 mg, 4.28 mmol, 1 eq.) and 5-benzyl 1-methyl (2S)-2-aminopentanedioate (1077.2 mg, 4.28 mmol, 1 eq.) in DMF (25 mL) were added HATU (3260.1 mg, 8.57 mmol, 2 eq.) and DIEA (2216.3 mg, 17.14 mmol, 4 eq.) and the resulting mixture was stirred at room temperature for overnight. The reaction mixture was quenched with water and extracted with EA. The extracts were washed with brine, dried over anhydrous sodium sulfate, filtered and concentrated. The residue was purified by flash to obtain 5-benzyl 1-methyl (2S)-2-{[1-(tert-butoxycarbonyl)-4-{[(9H-fluoren-9-ylmethoxy)carbonyl]amino}piperidin-4-yl]formamido}pentanedioate (2500 mg, 83.3%) as a yellow solid. MS: m/z: Calc'd for C₃₉H₄₅N₃O₉ [M+H−100]⁺600. found 600.

Step 3: To a stirred solution of 5-benzyl 1-methyl (2S)-2-{[1-(tert-butoxycarbonyl)-4-{[(9H-fluoren-9-ylmethoxy)carbonyl]amino}piperidin-4-yl]formamido}pentanedioate (2500 mg, 3.57 mmol, 1 eq.) in DCM (20 mL) was added DBU (1631.6 mg, 10.72 mmol, 3 eq.) and the resulting mixture was stirred at room temperature for overnight. The reaction mixture was quenched with water and extracted with EA. The extracts were washed with brine, dried over anhydrous sodium sulfate, filtered and concentrated. The residue was purified by reversed phase column to obtain 5-benzyl 1-methyl (2S)-2-{[4-amino-1-(tert-butoxycarbonyl)piperidin-4-yl]formamido}pentanedioate (1200 mg, 70.3%) as a white solid. MS: m/z: Calc'd for C₂₄H₃₅N₃O₇ [M+H]⁺478. found 478.

Step 4: The mixture of 5-benzyl 1-methyl (2S)-2-{[4-amino-1-(tert-butoxycarbonyl)piperidin-4-yl]formamido}pentanedioate (800 mg, 1.68 mmol, 1 eq.) in AcOH (1 mL) and Toluene (20 mL) was stirred at 60° C. for overnight. The reaction mixture was quenched with water and extracted with EA. The extracts were washed with brine, dried over anhydrous sodium sulfate, filtered and concentrated. The residue was purified by flash to obtain tert-butyl (3S)-3-[3-(benzyloxy)-3-oxopropyl]-2,5-dioxo-1,4,9-triazaspiro[5.5]undecane-9-carboxylate (350 mg, 46.9%) as a yellow solid. MS: m/z: Calc'd for C₂₃H₃₁N₃O₆ [M+H−56]⁺390. found 390.

Step 5: To a stirred solution of tert-butyl (3S)-3-[3-(benzyloxy)-3-oxopropyl]-2,5-dioxo-1,4,9-triazaspiro[5.5]undecane-9-carboxylate (300 mg, 0.67 mmol, 1 eq.) in MeOH (50 mL) was added Pd/C (358.3 mg, 3.36 mmol, 5 eq.) at hydrogen atmosphere, and the resulting mixture was stirred at room temperature for overnight. LCMS showed the reaction was completed. The reaction was filtered and washed with MeOH. The filtrate was concentrated and dried in vacuo to obtain 3-[(3S)-9-(tert-butoxycarbonyl)-2,5-dioxo-1,4,9-triazaspiro[5.5]undecan-3-yl]propanoic acid (200 mg, 83.5%) as a yellow oil. MS: m/z: Calc'd for C₁₆H₂₅N₃O₆ [M−H]⁺354. found 354.

Step 6: To a stirred solution of 3-[(3S)-9-(tert-butoxycarbonyl)-2,5-dioxo-1,4,9-triazaspiro[5.5]undecan-3-yl]propanoic acid (140 mg, 0.39 mmol, 1 eq.) in Toluene (15 mL) were added DPPA (216.8 mg, 0.78 mmol, 2 eq.) and TEA (159.5 mg, 1.57 mmol, 4 eq.) at 110° C. for 2 h under nitrogen atmosphere. The reaction was cooled to room temperature, benzyl alcohol (255.6 mg, 2.36 mmol, 6 eq.) was added, the mixture was stirred at 110° C. for overnight under nitrogen atmosphere. The reaction mixture was quenched with water and extracted with EA. The extracts were washed with brine, dried over anhydrous sodium sulfate, filtered and concentrated. The residue was purified by flash to obtain tert-butyl (3S)-3-(2-{[(benzyloxy)carbonyl]amino}ethyl)-2,5-dioxo-1,4,9-triazaspiro[5.5]undecane-9-carboxylate (60 mg, 33.1%) as a yellow solid. MS: m/z: Calc'd for C₂₃H₃₂N₄O₆ [M−H]⁺459. found 459.

Step 7: To a stirred solution of tert-butyl (3S)-3-(2-{[(benzyloxy)carbonyl]amino}ethyl)-2,5-dioxo-1,4,9-triazaspiro[5.5]undecane-9-carboxylate (70 mg, 0.15 mmol, 1 eq.) in MeOH (5 mL) was added Pd/C (80.8 mg, 0.76 mmol, 5 eq.) at hydrogen atmosphere for overnight. LCMS showed the reaction was completed. Then the mixture was filtered and washed with MeOH. Filtrate was concentrated and dried in vacuo to obtain tert-butyl (3S)-3-(2-aminoethyl)-2,5-dioxo-1,4,9-triazaspiro[5.5]undecane-9-carboxylate (35 mg, 70.5%) as a yellow oil. MS: m/z: Calc'd for C₁₅H₂₆N₄O₄ [M+H]⁺325. found 325.

Step 8: To a stirred solution of tert-butyl (3S)-3-(2-aminoethyl)-2,5-dioxo-1,4,9-triazaspiro[5.5]undecane-9-carboxylate (50 mg, 0.15 mmol, 1 eq.) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int B, 90.17 mg, 0.15 mmol, 1 eq.) in MeOH (4 mL) was added DIEA (59.4 mg, 0.46 mmol, 3 eq.) at room temperature for overnight. The reaction mixture was quenched with water and extracted with EA. The extracts were washed with brine, dried over anhydrous sodium sulfate, filtered and concentrated. The residue was purified by flash to obtain tert-butyl (3S)-3-{2-[({[(2R,3S,4S)-1-(tert-butoxycarbonyl)-4-[(tert-butoxycarbonyl)oxy]-2-[(4-methoxyphenyl)methyl]pyrrolidin-3-yl]oxy}carbonyl)amino]ethyl}-2,5-dioxo-1,4,9-triazaspiro[5.5]undecane-9-carboxylate (15 mg, 12.6%) as a white solid. MS: m/z: Calc'd for C₃₈H₅₇N₅O₁₂ [M+H−100]⁺676. found 676.

Step 9: To a stirred solution of tert-butyl (3S)-3-{2-[{[(2R,3S,4S)-1-(tert-butoxycarbonyl)-4-[(tert-butoxycarbonyl)oxy]-2-[(4-methoxyphenyl)methyl]pyrrolidin-3-yl]oxy}carbonyl)amino]ethyl}-2,5-dioxo-1,4,9-triazaspiro[5.5]undecane-9-carboxylate (15 mg, 0.02 mmol, 1 eq.) in DCM (2.5 mL) was added TFA (0.5 mL) at room temperature for 2 h. After completion of the reaction monitored by LCMS, the mixture was concentrated. The residue was purified by Prep-HPLC to obtain (2R,3S,4S)-4-hydroxy-2-[(4-methoxyphenyl)methyl]pyrrolidin-3-yl N-{2-[(3S)-2,5-dioxo-1,4,9-triazaspiro[5.5]undecan-3-yl]ethyl}carbamate; trifluoroacetic acid (2.1 mg, 17.26%) as a white solid. MS: m/z: Calc'd for C₂₃H₃₃N₅O₆ [M+H]⁺476. found 476. ¹H NMR (400 MHz, Methanol-d₄) δ 7.27-7.20 (m, 2H), 6.97-6.90 (m, 2H), 4.99-4.95 (m, 1H), 4.41 (d, J=4.2 Hz, 1H), 4.18-4.05 (m, 2H), 3.80 (s, 3H), 3.68-3.54 (m, 3H), 3.45-3.35 (m, 4H), 3.25 (dd, J=27.0, 9.9 Hz, 1H), 3.11 (dd, J=14.2, 7.2 Hz, 1H), 2.97 (dd, J=14.2, 8.6 Hz, 1H), 2.47-2.38 (m, 2H), 2.12-1.98 (m, 4H). Prep-HPLC-conditions: Column: Atlantis T3 Prep OBD Column, 19*250 mm, 5 μm; Mobile Phase A: Water (0.05% TFA), Mobile Phase B: ACN; Flow rate: 25 mL/min mL/min; Gradient: 3% B to 25% B in 10 min; Wave Length: 254 nm/220 nm; RT1(min): 9.15

### Synthetic Procedure of V187344

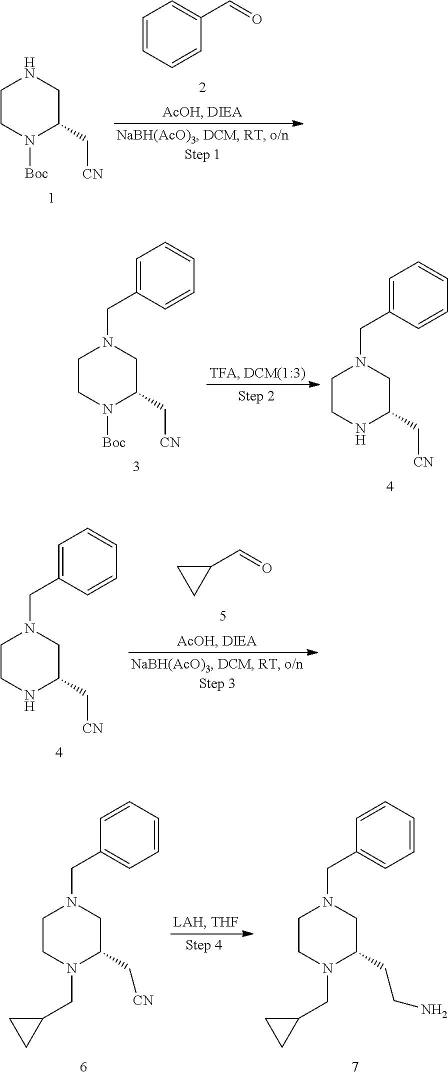

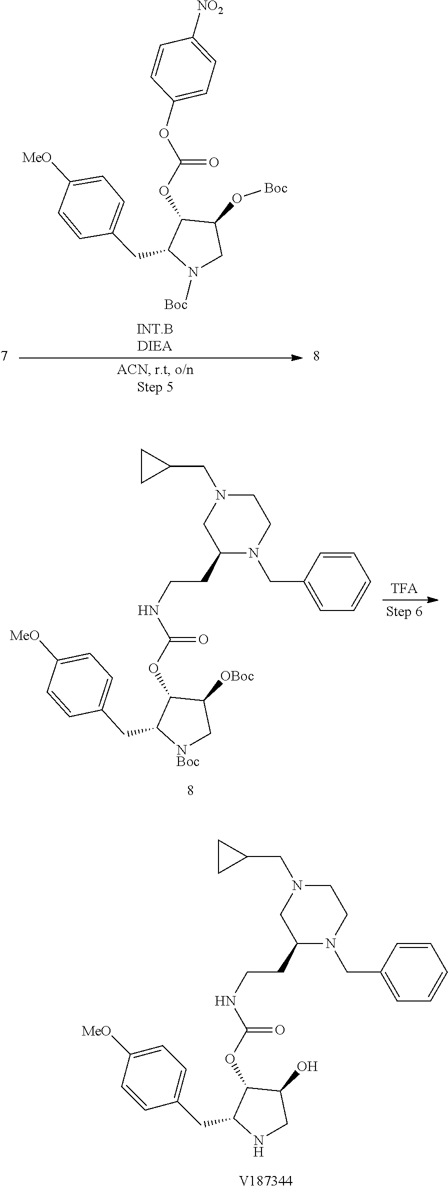

Step 1: To a solution of bis(tert-butyl (2S)-2-(cyanomethyl)piperazine-1-carboxylate) (3 g, 6.65 mmol, 1 equiv) in DCM (30 mL) was treated with bis(benzaldehyde) (2.12 g, 9.98 mmol, 1.5 equiv) for 5 mins at room temperature under nitrogen atmosphere followed by the addition of DIEA (2.6 g, 3 equiv) dropwise at room temperature. To the above mixture was added AcOH (1.6 g, 4 equiv) dropwise and the resulting mixture was stirred for 30 mins at room temperature. To the mixture was added NaBH(AcO)₃ (4.23 g, 19.97 mmol, 3 equiv) and the resulting mixture was stirred for 30 mins. The reaction mixture was concentrated under reduced pressure. The residue was purified by reversed-phase flash chromatography with the following conditions: column, C18 silica gel; mobile phase, NH₄HCO₃ in ACN, 10% to 50% gradient in 10 min; detector, UV 254 nm. afford bis(tert-butyl (2S)-4-benzyl-2-(cyanomethyl)piperazine-1-carboxylate) (3.5 g, 83.3%) as a white solid. MS: m/z: Calc'd for C₁₈H₂₅N₃O₂ [M+H]⁺316. Found, 316.

Step 2: To a stirred solution of tert-butyl (2S)-4-benzyl-2-(cyanomethyl)piperazine-1-carboxylate (1.8 g, 5.71 mmol, 1 equiv) in DCM (20 mL) was added TFA (5 mL) at room temperature under Nitrogen atmosphere. The resulting mixture was stirred for 2 h and then concentrated under vacuum. The crude product was used in the next step directly without further purification. MS: m/z: Calc'd for C₁₃H₁₇N₃ [M+H]⁺216. Found, 216.

Step 3: A solution of tris(2-[(2S)-4-benzylpiperazin-2-yl]acetonitrile) (400 mg, 0.62 mmol, 1 equiv) in DCM (10 mL) was treated with tris(4-formylpyridine) (239 mg, 0.74 mmol, 1.2 equiv) for 5 mins at room temperature under nitrogen atmosphere followed by the addition of DIEA (240 mg, 1.86 mmol, 2 equiv) and AcOH (223 mg, 3.72 mmol, 4 equiv). The mixture was stirred for 30 mins and NaBH(OAc)₃ (394 mg, 1.86 mmol, 2 equiv) was added. The reaction mixture was stirred for another 30 mins. and then was filtered and the filtrate was concentrated to give the crude product which was purified by reverse phase flash with the following conditions to afford tris(2-[(2S)-4-benzyl-1-(pyridin-4-ylmethyl)piperazin-2-yl]acetonitrile) (90 mg, 15.8%) as a white solid. MS: m/z: Calc'd for C₁₇H₂₃N₃[M+H]⁺270. Found, 270.

Step 4: A solution of 2-[(2S)-4-benzyl-1-(cyclopropylmethyl)piperazin-2-yl]acetonitrile (150 mg, 0.56 mmol, 1 equiv) in THF (5 mL) was added LAH (42 mg, 1.11 mmol, 2 equiv) at 0° C. under nitrogen atmosphere. The resulting mixture was stirred for 15 mins at room temperature. The mixture was diluted with EA, quenched with water. The aqueous layer was extracted with EA, dried and concentrated to give the crude product, which was used in the next step directly without further purification. MS: m/z: Calc'd for C₁₇H₂₇N₃[M+H]⁺274. Found, 274.

Step 5: A solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (80 mg, 0.14 mmol, 1.00 equiv) in ACN (3 mL) was added 2-[(2S)-4-benzyl-1-(cyclopropylmethyl)piperazin-2-yl]ethanamine (47 mg, 0.16 mmol, 1.2 equiv) at room temperature under nitrogen atmosphere followed by the addition of DIEA (35 mg, 0.27 mmol, 2 equiv). The resulting mixture was stirred at room temperature for overnight. The reaction was concentrated under vacuum and the resulting residue was purified by reversed-phase flash chromatography with the following conditions (ACN/H₂O, 0.1% NH₄HCO₃ in water) to afford tert-butyl (2R,3S,4S)-3-[({2-[(2S)-1-benzyl-4-(cyclopropylmethyl)piperazin-2-yl]ethyl}carbamoyl)oxy]-4-[(tert-butoxycarbonyl)oxy]-2-[(4-methoxyphenyl)methyl]pyrrolidine-1-carboxylate (40 mg, 40.7%) as a yellow solid. MS: m/z: Calc'd for C₄₀H₅₈N₄O₈ [M+H]⁺723. Found, 723.

Step 6: To the solution of tert-butyl (2R,3S,4S)-3-[({2-[(2S)-1-benzyl-4-(cyclopropylmethyl) piperazin-2-yl]ethyl}carbamoyl)oxy]-4-[(tert-butoxycarbonyl)oxy]-2-[(4-methoxyphenyl) methyl]pyrrolidine-1-carboxylate (40 mg, 0.06 mmol, 1 equiv) in DCM (3 mL) was added TFA (1 mL) at 0° C. The resulting mixture was stirred for 2 h and was concentrated under vacuum. The crude product (40 mg) was purified by Prep-HPLC to afford (2R,3S,4S)-4-hydroxy-2-[(4-methoxyphenyl)methyl]pyrrolidin-3-yl N-{2-[(2S)-1-benzyl-4-(cyclopropylmethyl)piperazin-2-yl]ethyl}carbamate (16.3 mg, 56.4%) as a white solid. MS: m/z: Calc'd for C₃₀H₄₂N₄O₄ [M+H]⁺523. Found, 523. ¹H NMR (400 MHz, DMSO-d₆) δ 7.36-7.26 (m, 4H), 7.24-7.21 (m, 1H), 7.15 (t, J=5.8 Hz, 1H), 7.12-7.05 (m, 2H), 6.85-6.78 (m, 2H), 4.99 (d, J=4.2 Hz, 1H), 4.54-4.48 (m, 1H), 3.91 (s, 1H), 3.71 (s, 3H), 3.52-3.44 (m, 1H), 3.38 (dd, J=13.2, 2.8 Hz, 1H), 3.29 (dd, J=7.3, 4.1 Hz, 1H), 3.10 (dd, J=11.5, 5.6 Hz, 1H), 3.05-2.81 (m, 3H), 2.64 (dd, J=13.5, 7.1 Hz, 1H), 2.56-2.53 (m, 2H), 2.50-2.43 (m, 3H), 2.38 (d, J=9.7 Hz, 1H), 2.26-2.05 (m, 3H), 1.61 (s, 2H), 0.80 (s, 1H), 0.43 (m, 2H), 0.06 (t, J=5.4 Hz, 2H). Prep-HPLC-conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min mL/min; Gradient: 23% B to 53% B in 13 min; Wave Length: 254 nm/220 nm nm; RT1(min): 7.98

### Synthetic Procedure of V187345

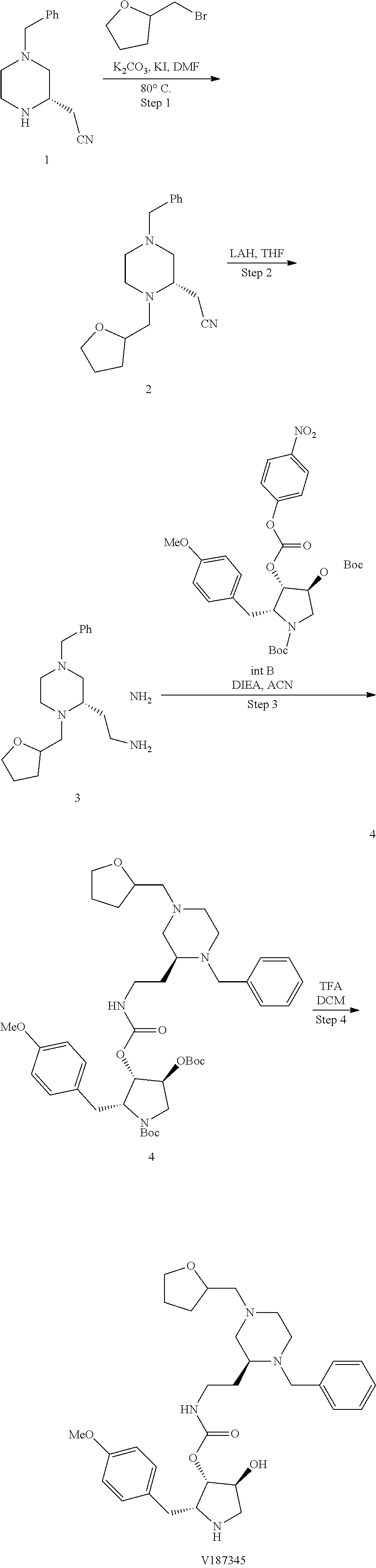

Step 1: To the stirred solution of 2-[(2S)-4-benzylpiperazin-2-yl]acetonitrile (400 mg, 1.86 mmol, 1 equiv) and 2-(bromomethyl)oxolane (307 mg, 1.86 mmol, 1 equiv) in DMF (5 ml) were added K₂CO₃ (514 mg, 3.72 mmol, 2 equiv) and KI (31 mg, 0.19 mmol, 0.1 equiv). The reaction mixture was stirred at 80° C. for 2 hours. Desired product could be detected by LCMS. The mixture was filtered and the filtrate was purified by reversed-phase flash chromatography with the following conditions: column, C18 silica gel; mobile phase, MeCN in Water (10 mmol/L NH₄HCO₃), 0% to 100% gradient in 25 min; detector, UV 254 nm. The resulting mixture was concentrated under reduced pressure to afford 2-[(2S)-4-benzyl-1-(oxolan-2-ylmethyl)piperazin-2-yl]acetonitrile (222 mg, 35.9%) as a white solid. MS: m/z: Calc'd for C₁₈H₂₅N₃O [M+H]⁺ 300. found [M+H]⁺ 300.

Step 2: To a stirred solution of 2-[(2S)-4-benzyl-1-(oxolan-2-ylmethyl)piperazin-2-yl]acetonitrile (200 mg, 0.67 mmol, 1 equiv) in THF (5 ml) was added LAH (1 mL in THF) dropwise at 0° C. under N2 atmosphere. The resulting reaction mixture was stirred at 0° C. for 2 hours. LCMS showed the reaction was completed. The reaction was quenched with MeOH at 0° C. The resulting mixture was concentrated under reduced pressure. This resulted in 2-[(2S)-4-benzyl-1-(oxolan-2-ylmethyl)piperazin-2-yl]ethanamine (150 mg, 74.0%) as a white solid. MS: m/z: Calc'd for C₁₈H₂₉N₃O [M+H]⁺ 304. found [M+H]⁺ 304.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.00 equiv) and 2-[(2S)-4-benzyl-1-(oxolan-2-ylmethyl)piperazin-2-yl]ethanamine (103 mg, 0.34 mmol, 2 equiv) in acetonitrile (5 ml) was added DIEA (88 mg, 0.68 mmol, 4 equiv). The reaction mixture was stirred at room temperature for overnight. Desired product could be detected by LCMS. The resulting mixture was concentrated under reduced pressure. This resulted in tert-butyl (2R,3S,4S)-3-[({2-[(2S)-1-benzyl-4-(oxolan-2-ylmethyl)piperazin-2-yl]ethyl}carbamoyl)oxy]-4-[(tert-butoxycarbonyl)oxy]-2-[(4-methoxyphenyl)methyl]pyrrolidine-1-carboxylate (100 mg, 39.1%) as a yellow solid. The crude product mixture was used in the next step directly without further purification. MS: m/z: Calc'd for C₄₁H₆₀N₄O₉[M+H]⁺ 753. Found, 753 [M+H]⁺.

Step 4: To a stirred solution of tert-butyl (2R,3S,4S)-3-[({2-[(2S)-1-benzyl-4-(oxolan-2-ylmethyl)piperazin-2-yl]ethyl}carbamoyl)oxy]-4-[(tert-butoxycarbonyl)oxy]-2-[(4-methoxyphenyl)methyl]pyrrolidine-1-carboxylate (50 mg, 0.066 mmol, 1 equiv) in DCM (5 ml) was added TFA (1 mL) dropwise at room temperature and the resulting mixture was stirred at room temperature for 1 hour. Desired product could be detected by LCMS. The mixture was concentrated to give the crude product (50 mg), which was purified by Prep-HPLC to afford (2R,3S,4S)-4-hydroxy-2-[(4-methoxyphenyl)methyl]pyrrolidin-3-yl N-{2-[(2S)-1-benzyl-4-(oxolan-2-ylmethyl)piperazin-2-yl]ethyl}carbamate (11 mg, 26.9%) as a white solid. MS: m/z: Calc'd for C₃₁H₄₄N₄O₅ [M+H]⁺ 553. Found, 553[M+H]⁺. ¹H NMR (400 MHz, Methanol-d₄) δ 7.45-7.32 (m, 5H), 7.27-7.19 (m, 2H), 6.97-6.88 (m, 2H), 5.00-4.94 (m, 1H), 4.40-4.33 (m, 1H), 4.27-4.11 (m, 2H), 3.97-3.86 (m, 2H), 3.84-3.74 (m, 5H), 3.60-3.52 (m, 2H), 3.46-3.33 (m, 2H), 3.27-3.17 (m, 4H), 3.14-3.03 (m, 3H), 3.01-2.86 (m, 2H), 2.86-2.81 (m, 2H), 2.21-1.86 (m, 5H), 1.68-1.55 (m, 1H). Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 29% B in 10 min; Wave Length: 254 nm/220 nm; RT1(min): 8.68

### Synthetic Procedure of V187381

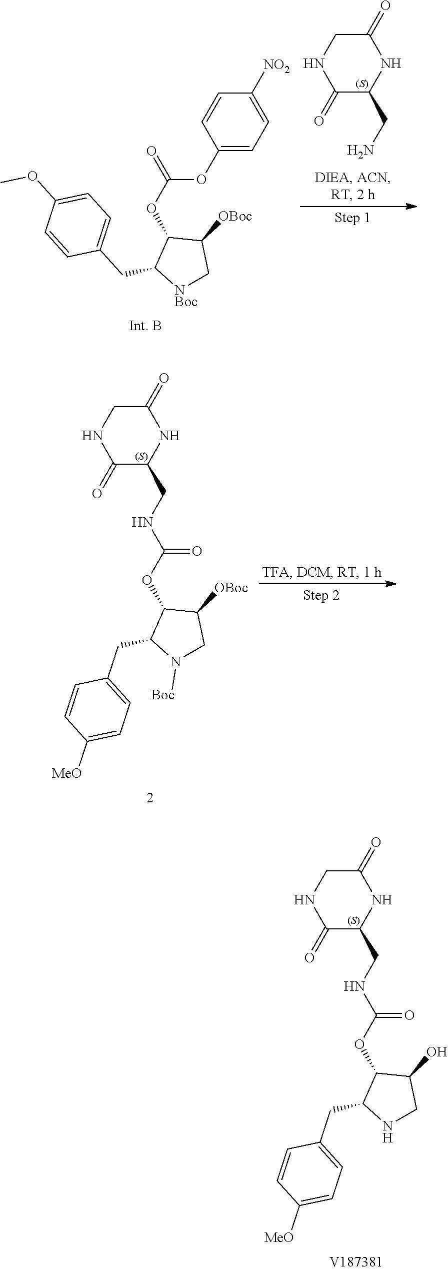

Step 1: To a stirred solution of (3S)-3-(aminomethyl)piperazine-2,5-dione (24.3 mg, 0.17 mmol, 1 equiv.) and tert-butyl (2R,3S,4S)-4-[(tert-butoxycarbonyl)oxy]-2-[(4-methoxyphenyl) methyl]-3-[(4-nitrophenoxycarbonyl)oxy]pyrrolidine-1-carboxylate (100 mg, 0.17 mmol, 1 equiv) in ACN (5 mL) was added DIEA (66 mg, 0.51 mmol, 3 equiv) at room temperature and was stirred for overnight at room temperature. The mixture was concentrated to give the crude product, which was purified by reversed-phase flash chromatography with the following conditions: column, C18 silica gel; mobile phase, MeCN in Water (0.1% TFA), 30% to 50% gradient in 10 min; detector, UV 254 nm. The resulting mixture was concentrated under vacuum. This resulted in tert-butyl (2R,3S,4S)-4-[(tert-butoxycarbonyl)oxy]-3-[({[(2S)-3,6-dioxopiperazin-2-yl]methyl}carbamoyl)oxy]-2-[(4-methoxyphenyl)methyl]pyrrolidine-1-carboxylate (36 mg, 35.7%) as a yellow solid. MS: m/z: Calc'd for C₂₈H₄₀N₄O₁₀ [M+H−100−56]⁺437. found 437.

Step 2: To a stirred solution of tert-butyl (2R,3S,4S)-4-[(tert-butoxycarbonyl)oxy]-3-[({[(2S)-3,6-dioxopiperazin-2-yl]methyl}carbamoyl)oxy]-2-[(4-methoxyphenyl)methyl]pyrrolidine-1-carboxylate (36 mg, 0.06 mmol, 1 equiv) in DCM (5 mL) was added TFA (1 mL) at 0° C. under N2 atmosphere. The resulting mixture was stirred for 1 h at room temperature. The mixture was concentrated under reduced pressure and the crude product (30 mg) was purified by Prep-HPLC to afford (2R,3S,4S)-4-hydroxy-2-[(4-methoxyphenyl)methyl]pyrrolidin-3-yl N-{[(2S)-3,6-dioxopiperazin-2-yl]methyl}carbamate (21.1 mg, 66.8%) as a white solid. MS: m/z: Calc'd for C₁₈H₂₄N₄O₆ [M+H]⁺393. found 393. ¹H NMR (400 MHz, Methanol-d₄) δ 7.30-7.19 (m, 2H), 6.98-6.90 (m, 2H), 4.97 (d, J=3.5 Hz, 1H), 4.43-4.37 (m, 1H), 4.22-4.05 (m, 2H), 4.04-3.88 (m, 2H), 3.80 (s, 3H), 3.69-3.52 (m, 3H), 3.21 (d, J=12.6 Hz, 1H), 3.18-3.07 (m, 1H), 2.93 (dd, J=14.2, 8.7 Hz, 1H). Prep-HPLC-conditions: Column: Atlantis T3 Prep OBD Column, 19*250 mm, 5 μm; Mobile Phase A: Water (0.05% TFA), Mobile Phase B: ACN; Flow rate: 25 mL/min mL/min; Gradient: 3% B to 25% B in 8 min; Wave Length: 254 nm/220 nm; RT1(min): 7.9

### Synthetic Procedure of V187382

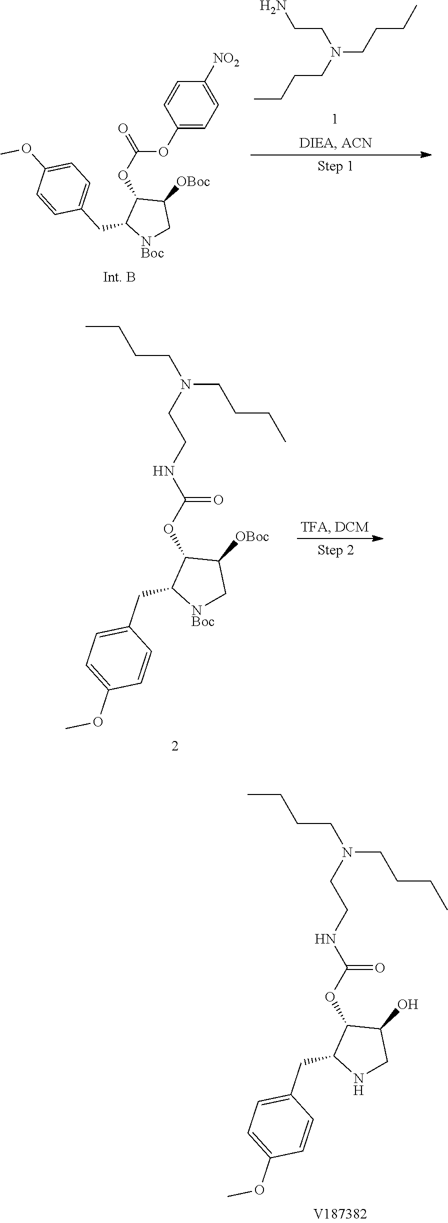

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq.) and (2-aminoethyl)dibutylamine (58.5 mg, 0.34 mmol, 2 eq.) in MeCN (3 mL) was added DIEA (88 mg, 0.68 mmol, 4 eq.). The resulting reaction mixture was stirred at ambient temperature for overnight. After completion of the reaction monitored by LCMS, the mixture was purified directly by reversed-phase column chromatography (0.05% TFA in water and acetonitrile) to afford tert-butyl (2R,3S,4S)-4-[(tert-butoxycarbonyl)oxy]-3-({[2-(dibutylamino)ethyl]carbamoyl}oxy)-2-[(4-methoxyphenyl)methyl]pyrrolidine-1-carboxylate (60 mg, 56.8%) as a white solid. MS: m/z: Calc'd for C₃₃H₅₅N₃O₈ [M+H]⁺623. Found, 623.

Step 2: To a stirred solution of (2R,3S,4S)-4-[(tert-butoxycarbonyl)oxy]-3-({[2-(dibutylamino) ethyl]carbamoyl}oxy)-2-[(4-methoxyphenyl)methyl]pyrrolidine-1-carboxylate (65 mg, 0.10 mmol) in DCM (3 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-[2-(dibutylamino)ethyl]carbamate (20.5 mg, 46.0%) as a white solid. MS: m/z: Calc'd for C₂₃H₃₉N₃O₄ [M+H]⁺422. Found, 422. ¹H NMR (400 MHz, Methanol-d₄) δ 7.28-7.20 (m, 2H), 6.93 (d, J=8.5 Hz, 2H), 5.01 (s, 1H), 4.40 (d, J=4.2 Hz, 1H), 4.22-4.14 (m, 1H), 3.80-3.79 (s, 3H), 3.57 (t, J=6.5 Hz, 3H), 3.35 (d, J=6.5 Hz, 2H), 3.27-3.18 (m, 5H), 3.11 (m, 1H), 2.99 (m, 1H), 1.79-1.66 (m, 4H), 1.44 (h, J=7.4 Hz, 4H), 1.02 (t, J=7.3 Hz, 6H). Prep-HPLC conditions: Column: XSelect CSH Prep C18 OBD Column, 5 um, 30*150 mm; Mobile Phase A: Water (0.05% TFA), Mobile Phase B: ACN; Flow rate: 60 mL/min; Gradient: 2 B to 30 B in 30 min; 254/220 nm; RT1:8.5; RT2: Injection Volume: ml; Number Of Runs:

### Synthetic Procedure of V187383

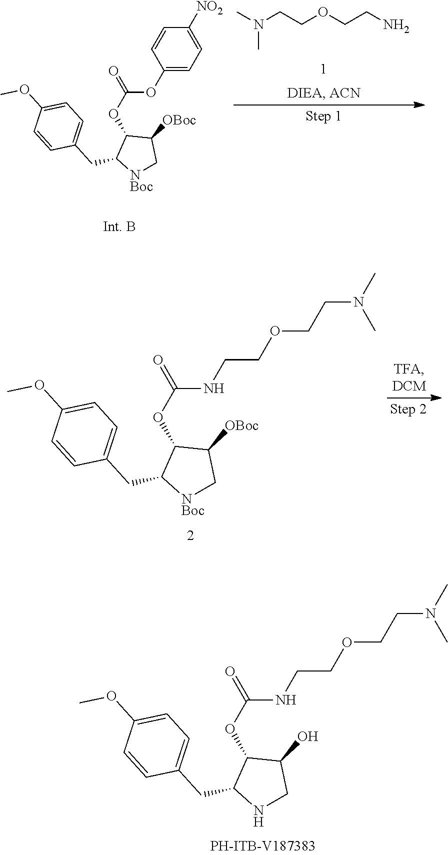

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq.) and [2-(2-aminoethoxy)ethyl]dimethylamine (44.9 mg, 0.34 mmol, 2 eq.) in MeCN (3 mL) was added DIEA (88 mg, 0.68 mmol, 4 eq.). The resulting reaction mixture was stirred at ambient temperature for overnight. After completion of the reaction monitored by LCMS, the mixture was purified directly by reversed-phase column chromatography (0.05% TFA in water and acetonitrile) to afford tert-butyl(2R,3S,4S)-4-[(tert-butoxycarbonyl)oxy]-3-[({2-[2-(dimethylamino)ethoxy]ethyl}carbamoyl)oxy]-2-[(4-methoxyphenyl)methyl]pyrrolidine-1-carboxylate (60 mg, 60.7%) as a white solid. MS: m/z: Calc'd for C₂₉H₄₇N₃O₉ [M+H]⁺582. Found 582.

Step 2: To a stirred solution of (2R,3S,4S)-4-[(tert-butoxycarbonyl)oxy]-3-[({2-[2-(dimethylamino)ethoxy]ethyl}carbamoyl)oxy]-2-[(4-methoxyphenyl)methyl]pyrrolidine-1-carboxylate (65 mg, 0.11 mmol) in DCM (3 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-{2-[2-(dimethylamino)ethoxy]ethyl}carbamate (21.2 mg, 49.2%) as a white solid. MS: m/z: Calc'd for C₁₉H₃₁N₃O₅ [M+H]⁺382. Found, 382. ¹H NMR (400 MHz, Methanol-d₄) δ 7.28-7.20 (m, 2H), 6.97-6.89 (m, 2H), 5.00-4.94 (m, 1H), 4.40-4.34 (m, 1H), 4.16-4.13 (m, 1H), 3.86-3.80 (m, 5H), 3.58-3.56 (m, 3H), 3.44-3.33 (m, 4H), 3.21-3.19 (m, 1H), 3.11-3.09 (m, 1H), 2.98-2.95 (m, 1H), 2.93 (s, 6H). Prep-HPLC conditions: Column: XSelect CSH Prep C18 OBD Column, 5 um, 30*150 mm; Mobile Phase A: Water (0.05% TFA), Mobile Phase B: ACN; Flow rate: 60 mL/min; Gradient: 2 B to 27 B in 24 min; 254/220 nm; RT1:8.68; RT2: Injection Volume: ml; Number Of Runs:

### Synthetic Procedure of V187384

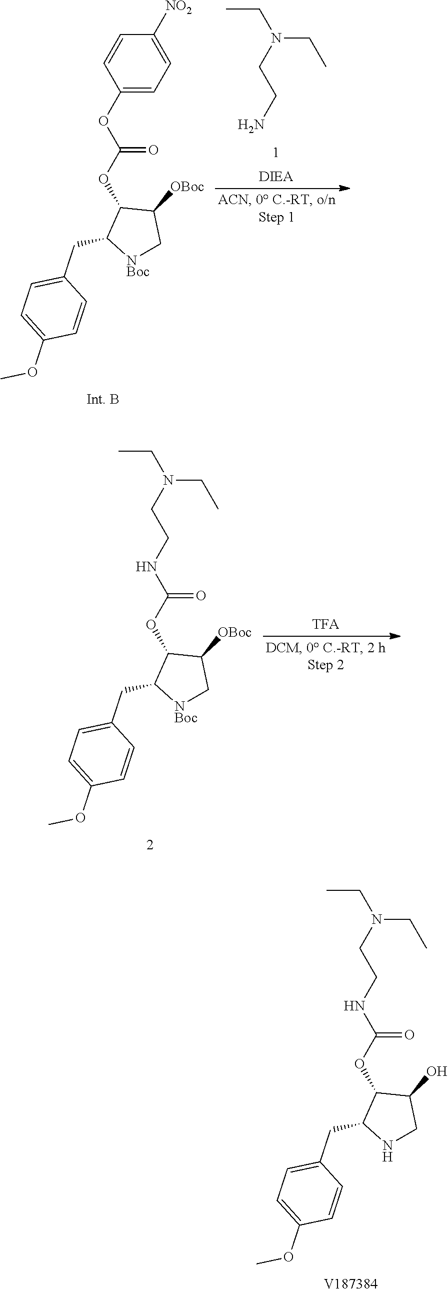

Step 1: To a stirred solution of (2R,3S,4S)-4-[(tert-butoxycarbonyl)oxy]-2-[(4-methoxyphenyl) methyl]pyrrolidin-3-yl 4-nitrophenyl carbonate (Int.B, 100 mg, 0.20 mmol, 1 eq.) and (2-aminoethyl)diethylamine (23.7 mg, 0.20 mmol, 1 eq.) in ACN (3 mL) were added DIEA (79.3 mg, 0.61 mmol, 3 eq.) at 0° C. The reaction was stirred at r.t. for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl)oxy]-3-({[2-(diethylamino)ethyl]carbamoyl}oxy)-2-[(4-methoxyphenyl) methyl]pyrrolidine-1-carboxylate (120 mg, 103.6%) as a yellow oil. MS: m/z: Calc'd for C₂₉H₄₇N₃O₈ [M−H]⁺ 566. Found, 566.

Step 2: To a stirred solution tert-butyl (2R,3S,4S)-4-[(tert-butoxycarbonyl)oxy]-3-({[2-(diethylamino)ethyl]carbamoyl}oxy)-2-[(4-methoxyphenyl)methyl]pyrrolidine-1-carboxylate (115 mg, 0.20 mmol, 1 eq.) in DCM (3 mL) were added TFA (1 mL) dropwise at 0° C. The reaction was stirred at r.t. for 2 h. After completion of the reaction monitored by LCMS. The resulting mixture was concentrated under reduced pressure. The crude product was purified by Prep-HPLC to afford (2R,3S,4S)-4-hydroxy-2-[(4-methoxyphenyl)methyl]pyrrolidin-3-yl N-[2-(diethylamino)ethyl]carbamate (14.5 mg, 19.1%) as a white semi-solid. MS: m/z: Calc'd for C₁₉H₃₁N₃O₄ [M+H]⁺366. Found, 366. ¹H NMR (400 MHz, Methanol-d₄) δ 7.28-7.21 (m, 2H), 6.98-6.90 (m, 2H), 5.05-4.99 (m, 1H), 4.42 (d, J=4.2 Hz, 1H), 4.19-4.15 (m, 1H), 3.80 (s, 3H), 3.62-3.53 (m, 3H), 3.37-3.27 (m, 6H), 3.22 (d, J=12.6 Hz, 1H), 3.12-3.08 (m, 6.7 Hz, 1H), 2.98-2.90 (m, 8.9 Hz, 1H), 1.36 (t, J=7.3 Hz, 6H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 17% B in 10 min; Wave Length: 254 nm/220 nm; RT1(min): 8.68

### Synthetic Procedure of V187385

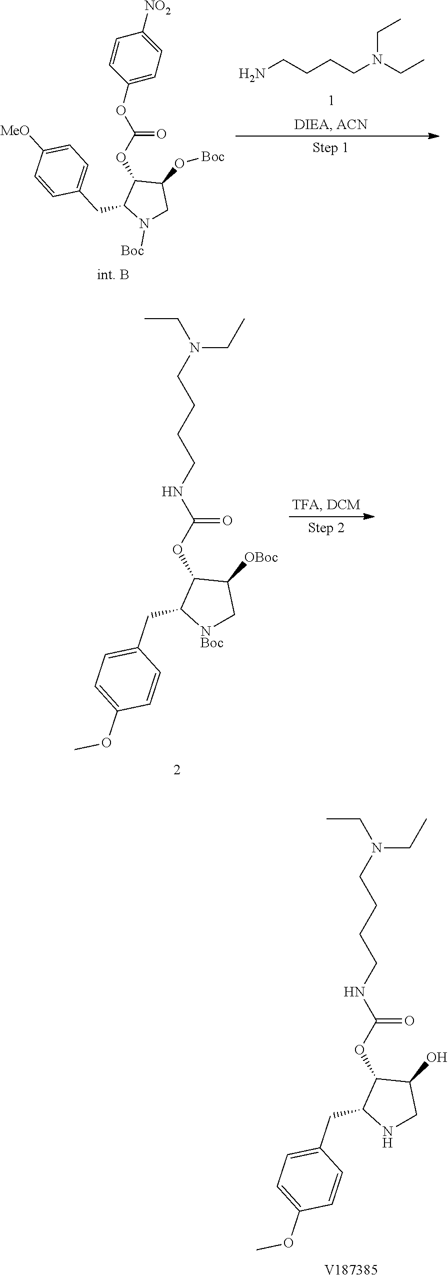

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq.) and (4-aminobutyl)diethylamine (49 mg, 0.34 mmol, 2 eq.) in MeCN (3 mL) was added DIEA (88 mg, 0.68 mmol, 4 eq.). The resulting reaction mixture was stirred at ambient temperature for overnight. After completion of the reaction monitored by LCMS, the mixture was purified directly by reversed-phase column chromatography (0.05% TFA in water and acetonitrile) to afford tert-butyl (2R,3S,4S)-4-[(tert-butoxycarbonyl)oxy]-3-({[4-(diethylamino)butyl]carbamoyl}oxy)-2-[(4-methoxyphenyl)methyl]pyrrolidine-1-carboxylate (80 mg, 55.5%) as a yellow solid. MS: m/z: Calc'd for C₃₁H₅₁N₃O₈ [M+H]⁺594. Found, 594.

Step 2: To a stirred solution of (2R,3S,4S)-4-[(tert-butoxycarbonyl)oxy]-3-({[4-(diethylamino) butyl]carbamoyl}oxy)-2-[(4-methoxyphenyl)methyl]pyrrolidine-1-carboxylate (100 mg, 0.17 mmol) in DCM (3 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-[4-(diethylamino)butyl]carbamate; trifluoroacetic acid (20 mg, 23.0%) as a colorless oil. MS: m/z: Calc'd for C₂₁H₃₅N₃O₄ [M+H]⁺394. Found, 394. ¹H NMR (400 MHz, Methanol-d₄) δ 7.28-7.20 (m, 2H), 6.97-6.89 (m, 2H), 4.98-4.93 (m, 1H), 4.37 (d, J=4.2 Hz, 1H), 4.21-4.11 (m, 1H), 3.79 (s, 3H), 3.62-3.53 (m, 1H), 3.29-3.15 (m, 9H), 3.15-3.06 (m, 1H), 3.03-2.92 (m, 1H), 1.82-1.70 (m, 2H), 1.70-1.58 (m, 2H), 1.32 (t, J=7.3 Hz, 6H). Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 3% B to 25% B in 10 min; Wave Length: 254 nm/220 nm; RT1(min): 8.68

### Synthetic Procedure of V187386

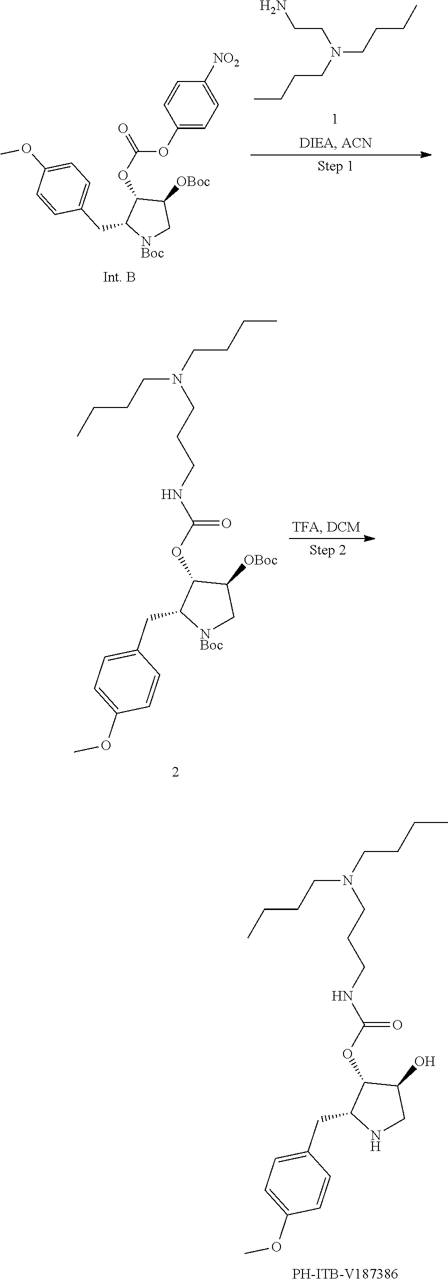

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq.) and (3-aminopropyl)dibutylamine (63.3 mg, 0.34 mmol, 2 eq.) in MeCN (3 mL) was added DIEA (88 mg, 0.68 mmol, 4 eq.). The resulting reaction mixture was stirred at ambient temperature for overnight. After completion of the reaction monitored by LCMS, the mixture was purified directly by reversed-phase column chromatography (0.05% TFA in water and acetonitrile) to afford (2R,3S,4S)-4-[(tert-butoxycarbonyl)oxy]-3-({[3-(dibutylamino)propyl]carbamoyl}oxy)-2-[(4-methoxyphenyl)methyl]pyrrolidine-1-carboxylate (60 mg, 55.5%) as a white solid. MS: m/z: Calc'd for C₃₄H₅₇N₃O₈ [M+H]⁺636. Found, 636.

Step 2: To a stirred solution of tert-butyl (2R,3S,4S)-4-[(tert-butoxycarbonyl)oxy]-3-({[3-(dibutylamino)propyl]carbamoyl}oxy)-2-[(4-methoxyphenyl)methyl]pyrrolidine-1-carboxylate (65 mg, 0.10 mmol) in DCM (3 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-[3-(dibutylamino) propyl]carbamate (29.0 mg, 64.6%) as a white solid. MS: m/z: Calc'd for C₂₄H₄₁N₃O₄ [M+H]⁺436. Found, 436. ¹H NMR (400 MHz, Methanol-d₄) δ 7.28-7.20 (m, 2H), 6.97-6.89 (m, 2H), 5.01-4.96 (m, 1H), 4.39 (d, J=4.2 Hz, 1H), 4.22-4.13 (m, 1H), 3.80 (s, 3H), 3.58 (m, 1H), 3.28-3.12 (m, 10H), 3.15-3.06 (m, 1H), 2.03-1.86 (m, 2H), 1.70-1.68 (m, 4H), 1.43 (d, J=7.4 Hz, 4H), 1.01 (t, J=7.4 Hz, 6H). Prep-HPLC conditions: Column: XSelect CSH Prep C18 OBD Column, 5 um, 30*150 mm; Mobile Phase A: Water (0.05% TFA), Mobile Phase B: ACN; Flow rate: 60 mL/min; Gradient: 2 B to 23 B in 60 min; 254/220 nm; RT1:8.5; RT2: Injection Volume: ml; Number Of Runs:

### Synthetic Procedure of V187387

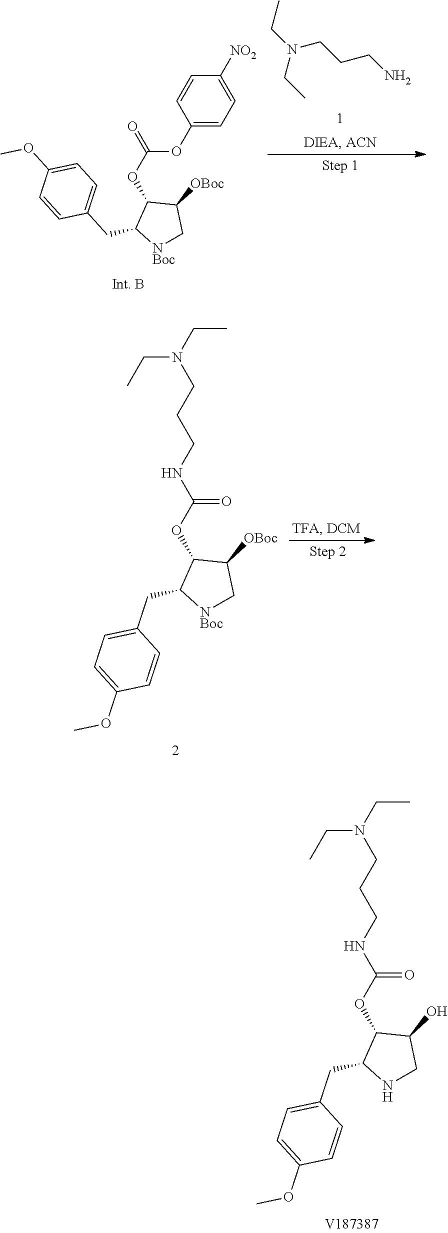

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1.0 eq.) and (3-aminopropyl)diethylamine (44.3 mg, 0.34 mmol, 2.0 eq.) in MeCN (3 mL) was added DIEA (65.9 mg, 0.51 mmol, 3.0 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl)oxy]-3-({[3-(diethylamino)propyl]carbamoyl}oxy)-2-[(4-methoxyphenyl)methyl]pyrrolidine-1-carboxylate (100 mg, 81.2%) as a little yellow oil. MS: m/z: Calc'd for C₃₀H₄₉N₃O₈ [M+H]⁺580. Found, 580.

Step 2: To a stirred solution of tert-butyl (2R,3S,4S)-4-[(tert-butoxycarbonyl)oxy]-3-({[3-(diethylamino)propyl]carbamoyl}oxy)-2-[(4-methoxyphenyl)methyl]pyrrolidine-1-carboxylate (55 mg, 0.10 mmol, 1.0 eq.) in DCM (2 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-[3-(diethylamino)propyl]carbamate trifluoroacetic acid salt (17.7 mg, 37.5%) as a light yellow oil. MS: m/z: Calc'd for C₂₀H₃₃N₃O₄ [M+H]⁺380. Found, 380. ¹H NMR (400 MHz, Methanol-d₄) δ 7.25 (d, J=8.3 Hz, 2H), 6.98-6.87 (m, 2H), 4.99 (d, J=3.4 Hz, 1H), 4.39 (d, J=4.1 Hz, 1H), 4.20-4.16 (m, 1H), 3.80 (s, 3H), 3.58 (dd, J=12.6, 4.3 Hz, 1H), 3.31-3.16 (m, 9H), 3.12 (dd, J=14.2, 6.9 Hz, 1H), 2.98 (dd, J=14.2, 8.7 Hz, 1H), 2.02-1.90 (m, 2H), 1.36-1.32 (m, 6H).

Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 18% B in 10 min; Wave Length: 254 nm/220 nm; RT1(min): 8.68

### Synthetic Procedure of V187436

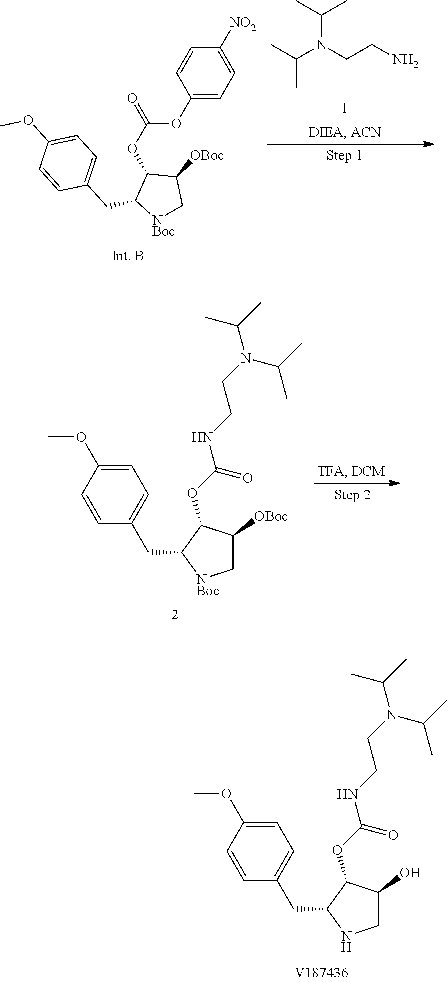

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq.) and (2-aminoethyl)diisopropylamine (49.0 mg, 0.34 mmol, 2 eq.) in MeCN (3 mL) was added DIEA (88 mg, 0.68 mmol, 4 eq.). The resulting reaction mixture was stirred at ambient temperature for overnight. After completion of the reaction monitored by LCMS, the mixture was purified directly by reversed-phase column chromatography (0.05% TFA in water and acetonitrile) to afford tert-butyl (2R,3S,4S)-4-[(tert-butoxycarbonyl)oxy]-3-({[2-(diisopropylamino)ethyl]carbamoyl}oxy)-2-[(4-methoxyphenyl)methyl]pyrrolidine-1-carboxylate (60 mg, 59.4%) as a white solid. MS: m/z: Calc'd for C₃₁H₅₁N₃O₈ [M+H]⁺594. Found 594.

Step 2: To a stirred solution of tert-butyl (2R,3S,4S)-4-[(tert-butoxycarbonyl)oxy]-3-({[2-(diisopropylamino)ethyl]carbamoyl}oxy)-2-[(4-methoxyphenyl)methyl]pyrrolidine-1-carboxylate (65 mg, 0.10 mmol) in DCM (3 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-[2-(diisopropylamino)ethyl]carbamate (19.8 mg, 45.8%) as a white solid. MS: m/z: Calc'd for C₂₁H₃₅N₃O₄ [M+H]⁺394. Found, 394. ¹H NMR (400 MHz, Methanol-d₄) δ 7.28-7.20 (m, 2H), 6.97-6.90 (m, 2H), 4.99 (d, J=3.6 Hz, 1H), 4.42-4.36 (m, 1H), 4.18-4.15 (m, 1H), 3.79-3.79 (m, 5H), 3.55-3.53 (m, 3H), 3.30 (s, 2H), 3.29 (t, J=7.3 Hz, 1H), 3.22-3.20 (m, 1H), 3.10-3.08 (m, 1H), 2.98-1.42 (m, 12H). Prep-HPLC conditions: Column: XSelect CSH Prep C18 OBD Column, 5 um, 30*150 mm; Mobile Phase A: Water (0.05% TFA), Mobile Phase B: ACN; Flow rate: 60 mL/min; Gradient: 2 B to 22 B in 30 min; 254/220 nm; RT1:8.5; RT2: Injection Volume: ml; Number Of Runs:

### Synthetic Procedure of V187437

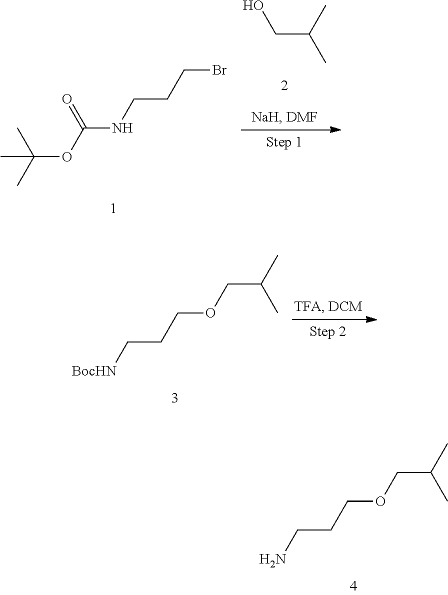

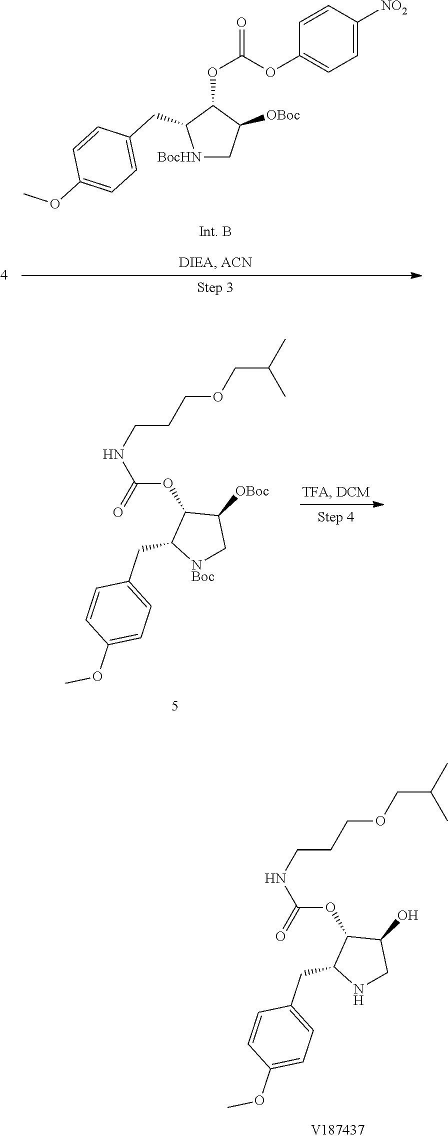

Step 1: To a stirred solution of isobutanol (373.5 mg, 5.04 mmol, 2.0 eq.) in DMF (10 mL) was added NaH (181.4 mg, 7.56 mmol, 3.0 eq.) at 0° C. and the resulting mixture was stirred for 0.5 hr. Then tert-butyl N-(3-bromopropyl)carbamate (600 mg, 2.52 mmol, 1.0 eq.) was added in portions at 0° C. The resulting reaction mixture was stirred at ambient temperature for overnight. After completion of the reaction monitored by LCMS. The reaction was quenched with Water at 0° C. The aqueous layer was extracted with EtOAc (3×10 mL). The combined organic layers were washed with brine and dried over anhydrous sodium sulfate. After filtration, the filtrate was concentrated under reduced pressure. This resulted in tert-butyl N-[3-(2-methylpropoxy)propyl]carbamate (400 mg, 68.6%) as a light yellow oil. MS: m/z: Calc'd for C₁₂H₂₅NO₃ [M+H]⁺232. Found, 232.

Step 2: To a stirred solution of tert-butyl N-[3-(2-methylpropoxy)propyl]carbamate (395 mg, 1.71 mmol, 1.0 eq.) in DCM (4 mL) was added TFA (2 mL) at room temperature. The resulting mixture was stirred at ambient temperature for 1 h. After completion of the reaction monitored by LCMS, the resulting mixture was concentrated under reduced pressure to afford 3-(2-methylpropoxy)propan-1-amine (200 mg, 89.3%) as a light yellow oil. MS: m/z: Calc'd for C₇H₁₇NO [M+H]⁺132. Found, 132.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.14 mmol, 1.0 eq.) and 3-(2-methylpropoxy)propan-1-amine (35.7 mg, 0.27 mmol, 2.0 eq.) in MeCN (3 mL) was added DIEA (52.7 mg, 0.41 mmol, 3.0 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl) oxy]-2-[(4-methoxyphenyl)methyl]-3-({[3-(2-methylpropoxy)propyl]carbamoyl}oxy) pyrrolidine-1-carboxylate (60 mg, 75.9%) as a white solid. MS: m/z: Calc'd for C₃₀H₄₈N₂O₉[M+H−56−56]⁺469. Found, 469.

Step 4: To a stirred solution of tert-butyl (2R,3S,4S)-4-[(tert-butoxycarbonyl)oxy]-2-[(4-methoxyphenyl)methyl]-3-({[3-(2-methylpropoxy)propyl]carbamoyl}oxy)pyrrolidine-1-carboxylate (60 mg, 0.10 mmol, 1.0 eq.) in DCM (2 mL) was added TFA (1 mL) at room temperature. The resulting mixture was stirred 1 h at 25° C. Desired product could be detected by LCMS. The resulting mixture was concentrated under reduced pressure. The crude product (60 mg) was purified by Prep-HPLC to afford (2R,3S,4S)-4-hydroxy-2-[(4-methoxyphenyl)methyl]pyrrolidin-3-yl N-[3-(2-methylpropoxy)propyl]carbamate (15 mg, 36.5%) as a yellow semi-solid. MS: m/z: Calc'd for C₂₀H₃₂N₂O₅ [M+H]⁺381. Found, 381. ¹H NMR (400 MHz, Methanol-d₄) δ 7.24 (dd, J=8.8, 2.9 Hz, 2H), 6.97-6.90 (m, 2H), 4.94 (d, J=3.7 Hz, 1H), 4.38 (d, J=4.0 Hz, 1H), 4.18-4.14 (m, 1H), 3.80 (d, J=2.8 Hz, 3H), 3.58-3.50 (m, 3H), 3.28-3.20 (m, 5H), 3.12 (dd, J=14.2, 7.2 Hz, 1H), 2.96 (dd, J=14.2, 8.6 Hz, 1H), 1.90-1.78 (m, 3H), 0.93 (d, J=6.5 Hz, 6H). Prep-HPLC-conditions: Column: Xselect CSH Prep Fluoro-Phenyl OBD C18 Column, 30*150 mm, 5 μm; Mobile Phase A: Water (0.05% TFA), Mobile Phase B: ACN; Flow rate: 60 m/min mL/min; Gradient: 10% B to 35% B in 8 min; Wave Length: 254 nm/220 nm; RT1(min): 7.07

### Synthetic Procedure of V187439

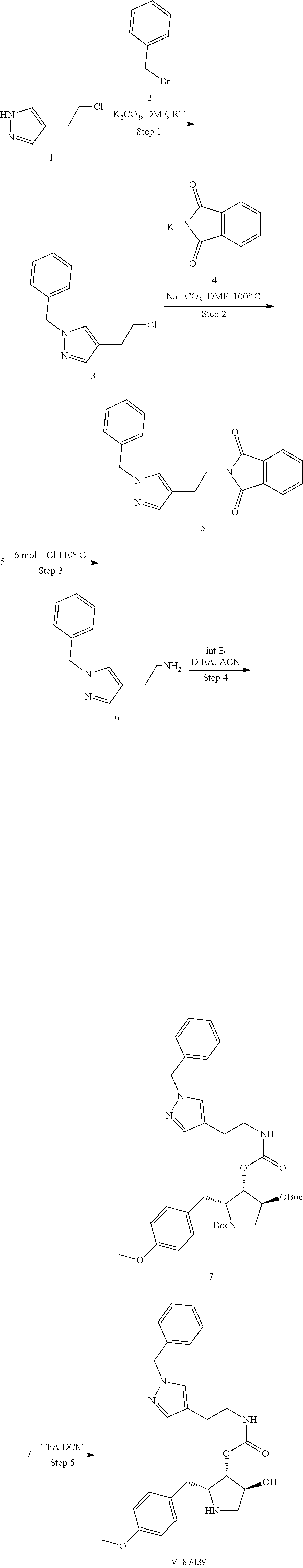

Step 1: To a stirred solution of 4-(2-chloroethyl)-1H-pyrazole (1 g, 7.66 mmol, 1 equiv) and benzyl bromide (1.57 g, 9.19 mmol, 1.2 equiv) in DMF (10 mL) was added K₂CO₃ (3.18 g, 22.97 mmol, 3 equiv). The reaction mixture was stirred at room temperature for overnight. Desired product could be detected by LCMS. The mixture was filtered and the filtrate was purified by reversed-phase flash chromatography with the following conditions: column, C18 silica gel; mobile phase, MeCN in Water (10 mmol/L NH4HCO3), 00% to 100% gradient in 25 min; detector, UV 254 nm. This resulted in 1-benzyl-4-(2-chloroethyl)pyrazole (1 g, 51.47%) as a white solid. MS: m/z: Calc'd for C₁₂H₁₃ClN₂ [M+H]⁺221. found 221.

Step 2: To a stirred solution of 1-benzyl-4-(2-chloroethyl)pyrazole (500 mg, 2.27 mmol, 1 equiv) and potassium phthalimide (503 mg, 2.72 mmol, 1.2 equiv) in DMF (5 ml) were added NaHCO₃ (571 mg, 6.80 mmol, 3 equiv). The reaction mixture was stirred at 100° C. for 2 hours. Desired product could be detected by LCMS. The mixture was filtered and the filtrate was purified by reversed-phase flash chromatography with the following conditions: column, C18 silica gel; mobile phase, MeCN in Water (10 mmol/L NH₄HCO₃), 0% to 100% gradient in 25 min; detector, UV 254 nm. The resulting mixture was concentrated under reduced pressure. This resulted in 2-[2-(1-benzylpyrazol-4-yl)ethyl]isoindole-1,3-dione (500 mg, 59.9%) as a white solid. MS: m/z: Calc'd for C₂₀H₁₇N₃O₂ [M+H]⁺332. found 332.

Step 3: To a stirred solution of 2-[2-(1-benzylpyrazol-4-yl)ethyl]isoindole-1,3-dione (500 mg, 0.60 mmol, 1 equiv, 40%) was added HCl (6M) (6 mL) in portions at room temperature under air atmosphere. The final reaction mixture was stirred at 110° C. for overnight. Desired product could be detected by LCMS. The mixture was concentrated under reduced pressure. This resulted in 2-(1-benzylpyrazol-4-yl)ethanamine (500 mg, 205.8%) as a white solid. The crude product mixture was used in the next step directly without further purification. MS: m/z: Calc'd for C₁₂H₁₅N₃[M+H]⁺202. Found, 202.

Step 4: To a stirred solution of 2-(1-benzylpyrazol-4-yl)ethanamine (68 mg, 0.34 mmol, 2 equiv) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.00 equiv) in acetonitrile (5 ml) was added DIEA (88 mg, 0.68 mmol, 4 equiv). The final reaction mixture was stirred at room temperature for overnight. Desired product could be detected by LCMS. The resulting mixture was concentrated under reduced pressure. This resulted in tert-butyl (2R,3S,4S)-3-({[2-(1-benzylpyrazol-4-yl)ethyl]carbamoyl}oxy)-4-[(tert-butoxycarbonyl)oxy]-2-[(4-methoxyphenyl)methyl]pyrrolidine-1-carboxylate (100 mg, 36.2%) as a white solid. The crude product mixture was used in the next step directly without further purification. MS: m/z: Calc'd for C₃₅H₄₆N₄O₈ [M+H]⁺651. Found, 651.

Step 5: To the solution of tert-butyl (2R,3S,4S)-3-({[2-(1-benzylpyrazol-4-yl)ethyl]carbamoyl}oxy)-4-[(tert-butoxycarbonyl)oxy]-2-[(4-methoxyphenyl)methyl]pyrrolidine-1-carboxylate (100 mg, 0.16 mmol) in DCM (5 mL) was added TFA (1 mL). The mixture was stirred at rt for 1 hour. LCMS showed the reaction was completed. The mixture was concentrated to give the crude product, which was purified by Prep-HPLC to afford the (2R,3S,4S)-4-hydroxy-2-(4-methoxybenzyl)pyrrolidin-3-yl (2-(1-benzyl-1H-pyrazol-4-yl)ethyl) carbamate (17.1 mg, 23.8%) as a white solid. MS: m/z: Calc'd for C₂₅H₃₀N₄O₄[M+H]⁺451. Found, 451. ¹H NMR (400 MHz, Methanol-d₄) δ 7.56 (s, 1H), 7.43 (s, 1H), 7.37-7.24 (m, 3H), 7.23-7.16 (m, 4H), 6.91 (d, J=8.5 Hz, 2H), 5.27 (s, 2H), 4.91 (d, J=3.4 Hz, 1H), 4.33 (d, J=4.2 Hz, 1H), 4.17-4.08 (m, 1H), 3.78 (s, 3H), 3.52 (dd, J=12.7, 4.3 Hz, 1H), 3.44-3.33 (m, 2H), 3.19 (d, J=12.6 Hz, 1H), 3.10-3.00 (m, 1H), 2.96-2.85 (m, 1H), 2.72 (t, J=7.0 Hz, 2H). Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 9% B to 39% B in 10 min; Wave Length: 254 nm/220 nm; RT1(min): 8.5

### Synthetic Procedure of V187465

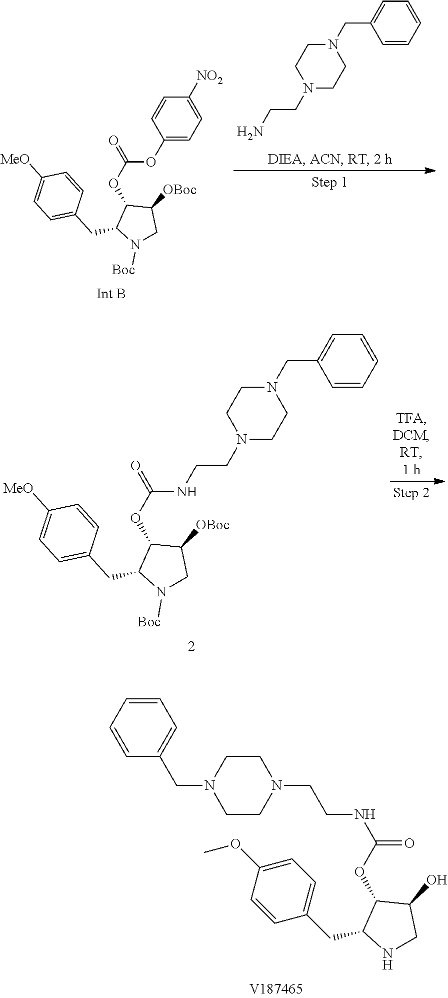

Step 1: To a stirred solution of 2-(4-benzylpiperazin-1-yl)ethanamine (45 mg, 0.20 mmol, 1.2 equiv) in ACN (5 mL) was added tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.00 equiv) at 0° C. under N2 atmosphere. The resulting mixture was stirred at room temperature for overnight. The mixture was concentrated under reduced pressure to afford tert-butyl (2R,3S,4S)-3-({[2-(4-benzylpiperazin-1-yl)ethyl]carbamoyl}oxy)-4-[(tert-butoxycarbonyl) oxy]-2-[(4-methoxyphenyl)methyl]pyrrolidine-1-carboxylate (120 mg, 96%) as a yellow oil. MS: m/z: Calc'd for C₃₆H₅₂N₄O₈ [M+H]⁺669. found 669.

Step 2: To a stirred solution of tert-butyl (2R,3S,4S)-3-({[2-(4-benzylpiperazin-1-yl)ethyl]carbamoyl}oxy)-4-[(tert-butoxycarbonyl)oxy]-2-[(4-methoxyphenyl)methyl]pyrrolidine-1-carboxylate (200 mg, 0.29 mmol, 1 equiv) in DCM (5 mL) was added TFA (1 mL) at 0° C. The resulting mixture was stirred for 1 h at room temperature and the resulting mixture was concentrated under vacuum. The crude product (100 mg) was purified by Prep-HPLC to afford (2R,3S,4S)-4-hydroxy-2-[(4-methoxyphenyl)methyl]pyrrolidin-3-yl N-[2-(4-benzylpiperazin-1-yl)ethyl]carbamate (32.7 mg, 22.7%) as a white solid. MS: m/z: Calc'd for C₂₅H₃₄FNO₈ [M+H]⁺469. found 469. ¹H NMR (400 MHz, Methanol-d₄) δ 7.45 (s, 5H), 7.29-7.20 (m, 2H), 6.97-6.89 (m, 2H), 5.02-4.96 (m, 1H), 4.42-4.34 (m, 1H), 4.21-4.11 (m, 3H), 3.79 (s, 3H), 3.57 (dd, J=12.7, 4.3 Hz, 1H), 3.51-3.33 (m, 3H), 3.22-3.03 (m, 9H), 3.02-2.90 (m, 1H), 2.91 (s, 2H). Prep-HPLC-conditions: Column: XBridge Prep Shield RP18 OBD C18 Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm; RT1(min): 8.5

### Synthetic Procedure of V187507

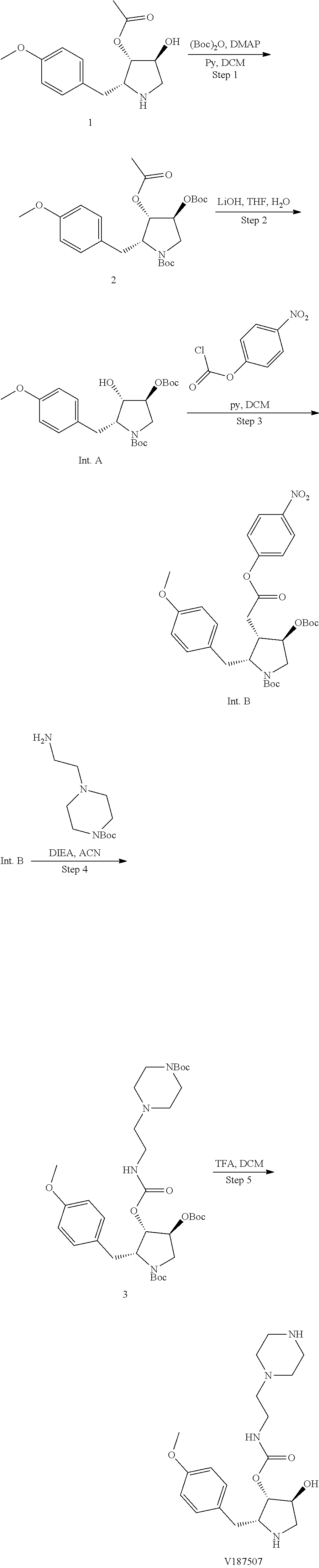

Step 1: To a stirred solution of anisomycin (5 g, 18.85 mmol, 1 eq.) in Pyridine (100 mL) was added Boc₂O (41.13 g, 188.46 mmol, 10 eq.) and DMAP (3.45 g, 28.27 mmol, 1.5 eq.) at room temperature. The resulting mixture was stirred at 25° C. for overnight. Upon completion, the reaction mixture was concentrated in vacuo. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 220 nm. This resulted in tert-butyl (2R,3S,4S)-3-(acetyloxy)-4-[(tert-butoxycarbonyl)oxy]-2-[(4-methoxyphenyl)methyl]pyrrolidine-1-carboxylate (8 g, 91.2%) as a light yellow oil. MS: m/z: Calc'd for C₂₄H₃₅NO₈ [M+H−56−100]⁺310. found 310.

Step 2: To a stirred solution of tert-butyl (2R,3S,4S)-3-(acetyloxy)-4-[(tert-butoxycarbonyl)oxy]-2-[(4-methoxyphenyl)methyl]pyrrolidine-1-carboxylate (8 g, 17.18 mmol, 1 eq.) in THF (100 mL) was added LiOH (1.23 g, 51.55 mmol, 3 eq.) and H₂O (10 mL) at room temperature. The resulting mixture was stirred for overnight. The resulting mixture was extracted with EtOAc (3×30 mL). The combined organic layers were washed with water (3×10 mL), dried over anhydrous Na₂SO₄. After filtration, the filtrate was concentrated under reduced pressure to afford crude product tert-butyl (2R,3S,4S)-4-[(tert-butoxycarbonyl)oxy]-3-hydroxy-2-[(4-methoxyphenyl)methyl]pyrrolidine-1-carboxylate (7.2 g, 98.9%) as a light yellow solid, which was used directly in the next step without further purification. MS: m/z: Calc'd for C₂₂H₃₃NO₇ [M+H−56−56]⁺312. found 312.

Step 3: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (4.7 g, 11.10 mmol, 1 eq.) and 4-nitrophenyl carbonochloridate (3.36 g, 16.67 mmol, 1.5 eq.) in DCM (50 mL) was added Pyridine (1.76 g, 22.25 mmol, 2 eq.) dropwise at room temperature under nitrogen atmosphere. The resulting mixture was stirred at room for 2 h. After completion of reaction monitored by LCMS. The residue was purified by silica gel column chromatography, eluted with PE/EA (5:1) to afford tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (5.4 g, 82.7%) as a light yellow oil. MS: m/z: Calc'd for C₂₉H₃₆N₂O₁₁ [M+H−100]⁺489. found 489.

Step 4: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (70 mg, 0.12 mmol, 1 eq.) and tert-butyl 4-(2-aminoethyl)piperazine-1-carboxylate (27.3 mg, 0.12 mmol, 1 eq.) in ACN (5 mL) was added DIEA (46.1 mg, 0.36 mmol, 3 eq.) dropwise at room temperature under nitrogen atmosphere. The resulting mixture was stirred for 12 h. Desired product could be detected by LCMS. The crude product was used in the next step directly without further purification. MS: m/z: Calc'd for C₃₄H₅₄N₄O₁₀ [M+H]⁺679. found 679.

Step 5: To a stirred solution of tert-butyl 4-{2-[({[(2R,3S,4S)-1-(tert-butoxycarbonyl)-4-[(tert-butoxycarbonyl)oxy]-2-[(4-methoxyphenyl)methyl]pyrrolidin-3-yl]oxy}carbonyl)amino]ethyl}piperazine-1-carboxylate (90 mg, 0.13 mmol, 1 equiv) in DCM (5 mL) was added TFA (1 mL) at room temperature under nitrogen atmosphere. The resulting mixture was stirred for 2 h. After removing the solvent, the crude product (90 mg) was purified by Prep-HPLC to afford (2R,3S,4S)-4-hydroxy-2-[(4-methoxyphenyl)methyl]pyrrolidin-3-ylN-[2-(piperazin-1-yl)ethyl]carbamate; trifluoroacetic acid (29.5 mg, 45.1%) as a light yellow solid. MS: m/z: Calc'd for C₁₉H₃₀N₄O₄ [M+H]⁺379. found 379. ¹H NMR (400 MHz, Methanol-d₄) δ 7.25 (d, J=8.3 Hz, 2H), 6.94 (d, J=8.2 Hz, 2H), 4.99 (d, J=3.4 Hz, 1H), 4.40 (d, J=4.1 Hz, 1H), 4.17 (s, 1H), 3.80 (d, J=1.1 Hz, 3H), 3.57 (dd, J=12.6, 4.3 Hz, 1H), 3.34 (d, J=14.9 Hz, 2H), 3.30-3.18 (m, 5H), 3.16-3.08 (m, 1H), 2.96 (dd, J=14.2, 8.7 Hz, 1H), 2.83 (s, 4H), 2.66 (m, 2H). Prep-HPLC-conditions: Column: Xselect CSH Prep C₁₈ Column, 30*150 mm, 5 μm; Mobile Phase A: Water (0.05% TFA), Mobile Phase B: ACN; Flow rate: 60 mL/min mL/min; Gradient: 2% B to 17% B in 10 min; Wave Length: 254 nm/220 nm nm; RT1(min): 8.68

### Synthetic Procedure of V187508

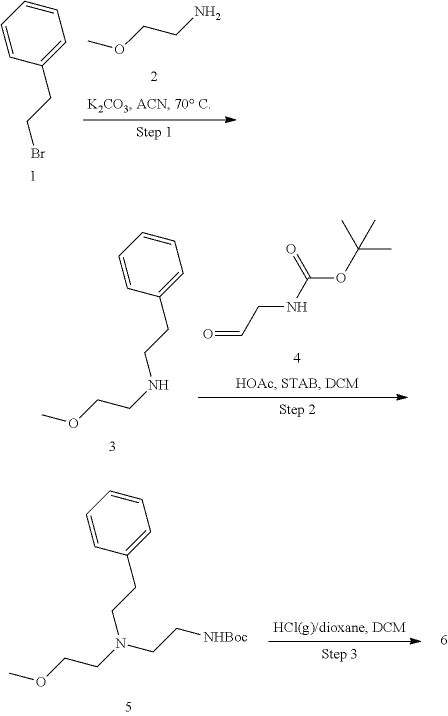

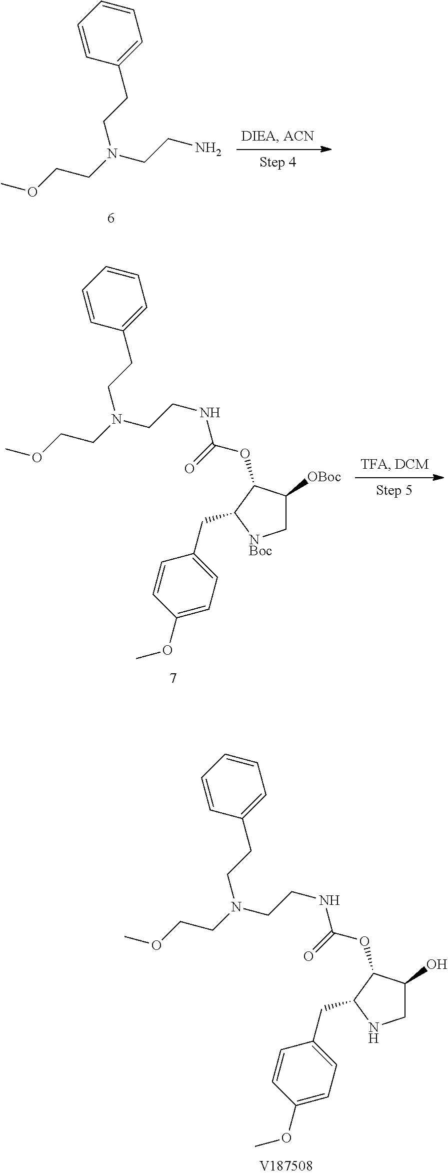

Step 1: To the solution of (2-bromoethyl)benzene (500 mg, 2.70 mmol, 1 eq.) and ethanamine, 2-methoxy- (244 mg, 3.24 mmol, 1.2 eq.) in ACN (5 mL) was added K₂CO₃ (747 mg, 5.40 mmol, 2 eq.). The resulting mixture was stirred at 80° C. for overnight. Upon completion. The mixture was filtered and the filtrate was concentrated to give the crude product which was used directly in the next step without further purification. MS: m/z: Calc'd for C₁₁HINO [M+H]⁺180. found 180.

Step 2: To the solution of (2-methoxyethyl)(2-phenylethyl)amine (200 mg, 1.12 mmol, 1 eq.) and tert-butyl N-(2-oxoethyl)carbamate (355 mg, 2.23 mmol, 2 eq.) in DCM (10 mL) was added HOAc (6.70 mg, 0.11 mmol, 0.1 eq.). The mixture was stirred at r.t. for 1 h before NaBH(OAc)₃ (709 mg, 3.35 mmol, 3 eq.) was added. The result mixture was stirred for another 1 h. Upon completion. The mixture was filtered and concentrated to give the crude product, which was purified by reversed phase chromatography to afford tert-butyl N-{2-[(2-methoxyethyl)(2-phenylethyl)amino]ethyl}carbamate (136 mg, 37.8%) as a light yellow oil. MS: m/z: Calc'd for C₁₈H₃₀N₂O₃[M+H]⁺323. found 323.

Step 3: To the solution of tert-butyl N-{2-[(2-methoxyethyl)(2-phenylethyl)amino]ethyl}carbamate (130 mg, 0.40 mmol) in DCM (2 mL) was added TFA (2 mL). The mixture was stirred at r.t. for 1 h. Upon completion. The mixture was concentrated to give the crude product, which was used directly in the next step without further purification. MS: m/z: Calc'd for C₁₃H₂₂N₂O [M+H]⁺223. found 223.

Step 4: To the solution of (2-aminoethyl)(2-methoxyethyl)(2-phenylethyl)amine (45 mg, 0.20 mmol, 1.2 eq.) and tert-butyl (2R,3S,4S)-4-[(tert-butoxycarbonyl)oxy]-2-[(4-methoxyphenyl) methyl]-3-[(4-nitrophenoxycarbonyl)oxy]pyrrolidine-1-carboxylate (100 mg, 0.17 mmol, 1 eq.) in ACN (3 mL) was added DIEA (44 mg, 0.34 mmol, 2 eq.). The mixture was stirred at r.t. for 2 h. Upon completion. The mixture was concentrated to give the crude product which was used directly in the next step without further purification. MS: m/z: Calc'd for C₃₆H₅₃N₃O₉ [M+H]⁺672. found 672.

Step 5: To the solution of tert-butyl (2S,3S,4R)-4-[(tert-butoxycarbonyl)oxy]-3-[({2-[(2-methoxyethyl)(2-phenylethyl)amino]ethyl}carbamoyl)oxy]-2-[(4-methoxyphenyl) methyl]pyrrolidine-1-carboxylate (100 mg, 0.15 mmol) in DCM (5 mL) was added TFA (1 mL) slowly. The mixture was stirred at r.t. for 1 h. Upon completion. The mixture was concentrated to give the crude product, which was purified by Prep HPLC to obtained (2S,3S,4R)-4-hydroxy-2-[(4-methoxyphenyl)methyl]pyrrolidin-3-yl N-{2-[(2-methoxyethyl)(2-phenylethyl)amino]ethyl}carbamate as a brown solid. MS: m/z: Calc'd for C₂₆H₃₇N₃O₅ [M+H]⁺472. found 472. ¹H NMR (400 MHz, Methanol-d₄) δ 7.38-7.25 (m, 5H), 7.23 (d, 2H), 6.92 (d, 2H), 5.03-4.97 (m, 1H), 4.39 (d, J=4.2 Hz, 1H), 4.23-4.13 (m, 1H), 3.82-3.77 (m, 2H), 3.77 (s, 3H), 3.64-3.41 (m, 9H), 3.43 (s, 3H), 3.21 (d, J=12.7 Hz, 1H), 3.15-3.04 (m, 3H), 3.03-2.93 (m, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 4% B to 28% B in 10 min; Wave Length: 254 nm/220 nm; RT1(min): 8.68

### Synthetic Procedure of V187509

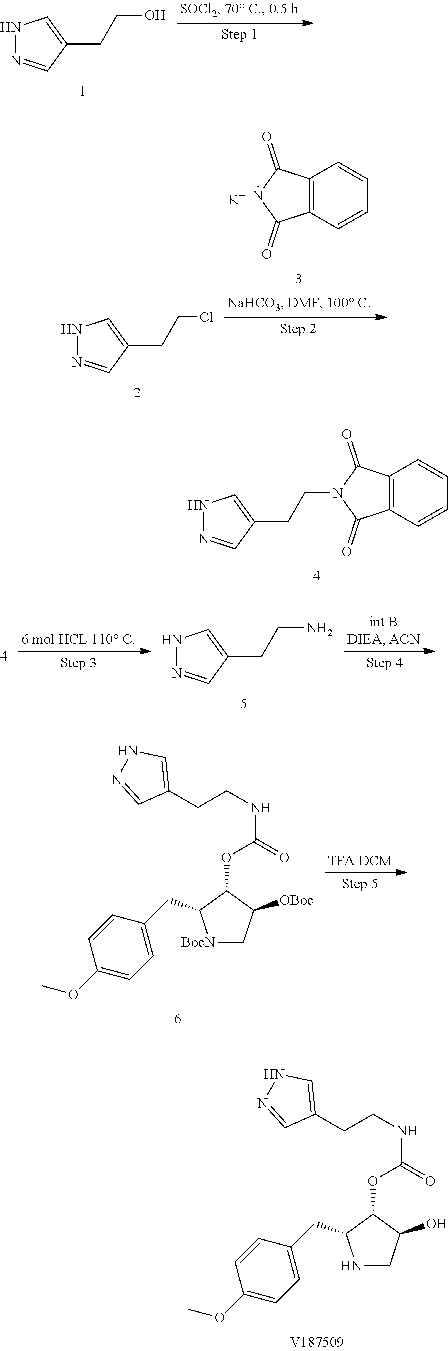

Step 1: The solution of 2-(1H-pyrazol-4-yl)ethanol (1 g, 8.92 mmol, 1 equiv) in SOCl₂ (5 mL) was stirred at 70° C. for 0.5 h. TLC showed the starting material was consumed. The mixture was concentrated under reduced pressure to afford 4-(2-chloroethyl)-1H-pyrazole (1.2 g, 92.74%) as a yellow solid. The crude product mixture was used in the next step directly without further purification.

Step 2: To a stirred solution of 4-(2-chloroethyl)-1H-pyrazole (400 mg, 3.06 mmol, 1 equiv) and potassium phthalimide (681 mg, 3.68 mmol, 1.2 equiv) in DMF (5 ml) was added NaHCO₃ (221 mg, 9.19 mmol, 3 equiv). The final reaction mixture was stirred at 100° C. for 2 hours. Desired product could be detected by LCMS. The mixture was filtered and the filtrate was purified by reversed-phase flash chromatography with the following conditions: column, C18 silica gel; mobile phase, MeCN in Water (10 mmol/L NH₄HCO₃), 0% to 100% gradient in 25 min; detector, UV 254 nm. The resulting mixture was concentrated under reduced pressure. This resulted in 2-[2-(1H-pyrazol-4-yl)ethyl]isoindole-1,3-dione (400 mg, 48.7%) as a white solid. MS: m/z: Calc'd for C₁₃H₁₁N₃O₂ [M+H]⁺ 242. found 242.

Step 3: A solution of 2-[2-(1H-pyrazol-4-yl)ethyl]isoindole-1,3-dione (400 mg, 1.66 mmol, 1 equiv) in HCl (6 M) (6 mL) was stirred for overnight at 110° C. under air atmosphere. Desired product could be detected by LCMS. The resulting mixture was concentrated under reduced pressure. This resulted in 2-(1H-pyrazol-4-yl)ethanamine (350 mg, 170.93%) as a white solid. The crude product mixture was used in the next step directly without further purification. MS: m/z: Calc'd for C₅H₉N₃ [M+H]⁺112. Found, 112.

Step 4: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.00 equiv) and 2-(1H-pyrazol-4-yl)ethanamine (38 mg, 0.34 mmol, 2 equiv) in acetonitrile (5 ml) was added DIEA (88 mg, 0.68 mmol, 4 equiv). The resulting reaction mixture was stirred at rt for 2 h. Desired product could be detected by LCMS. The resulting mixture was concentrated under reduced pressure to afford tert-butyl (2R,3S,4S)-4-[(tert-butoxycarbonyl) oxy]-2-[(4-methoxyphenyl)methyl]-3-({[2-(1H-pyrazol-4-yl)ethyl]carbamoyl}oxy)pyrrolidine-1-carboxylate (100 mg, 52.5%) as a yellow solid. The crude product mixture was used in the next step directly without further purification. MS: m/z: Calc'd for C₂₈H₄₀N₄O₈ [M+H]⁺561. Found, 561.

Step 5: To a stirred solution of tert-butyl (2R,3S,4S)-4-[(tert-butoxycarbonyl)oxy]-2-[(4-methoxyphenyl)methyl]-3-({[2-(1H-pyrazol-4-yl)ethyl]carbamoyl}oxy)pyrrolidine-1-carboxylate (100 mg, 0.178 mmol, 1 equiv) in DCM (5 ml) was added TFA (1 mL, 13.463 mmol, 75.48 equiv) dropwise. The reaction mixture was stirred at room temperature for 1 hour. LCMS showed the reaction was complete. The crude product (50 mg) was purified by Prep-HPLC to afford (2R,3S,4S)-4-hydroxy-2-[(4-methoxyphenyl)methyl]pyrrolidin-3-yl N-[2-(1H-pyrazol-4-yl)ethyl]carbamate (23.9 mg, 35.6%) as a white solid. MS: m/z: Calc'd for C₁₈H₂₄N₄O₄ [M+H]⁺361. Found, 361. ¹H NMR (400 MHz, Methanol-d₄) δ 7.56 (d, J=2.9 Hz, 2H), 7.26-7.14 (m, 2H), 6.97-6.87 (m, 2H), 4.92 (d, J=3.6 Hz, 1H), 4.35 (d, J=4.2 Hz, 1H), 4.18-4.09 (m, 1H), 3.79 (s, 3H), 3.59-3.50 (m, 1H), 3.45-3.32 (m, 2H), 3.20 (d, J=12.7 Hz, 1H), 3.13-3.03 (m, 1H), 2.97-2.87 (m, 1H), 2.76 (t, J=7.0 Hz, 2H). Prep-HPLC conditions: Column: Xselect CSH Prep C18 OBD Column, 19*250 mm, 5 μm; Mobile Phase A: Water (0.05% TFA), Mobile Phase B: ACN; Flow rate: 25 mL/min mL/min; Gradient: 14% B to 30% B in 10 min; Wave Length: 254 nm/220 nm; RT1(min): 9.6.

### Synthetic Procedure of V187516

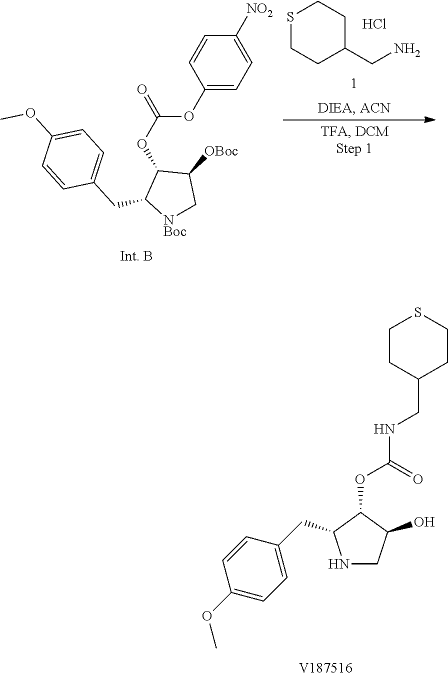

To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq) and 1-(thian-4-yl) methanamine hydrochloride (57 mg, 0.34 mmol, 2 eq) in ACN (10 mL) was added DIEA (66 mg, 0.51 mmol, 3 eq). The resulting reaction mixture was stirred at ambient temperature for overnight. After completion of the reaction monitored by LCMS, the mixture was concentrated under reduced pressure. To the mixture added DCM (5 mL) and TFA (5 mL) at 0° C. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl N-(thian-4-ylmethyl) carbamate (12.9 mg, 19.5%) as a white solid. MS: m/z: Calc'd for C₁₉H₂₈N₂O₄S [M+H]⁺381. found 381. 1H NMR (400 MHz, DMSO-d₆) δ 7.26 (t, J=6.0 Hz, 1H), 7.12-7.06 (m, 2H), 6.81 (d, J=8.5 Hz, 2H), 4.99 (s, 1H), 4.48 (dd, J=3.9, 1.7 Hz, 1H), 3.95-3.89 (m, 1H), 3.71 (s, 3H), 3.36-3.26 (m, 1H), 3.12 (dd, J=11.5, 5.6 Hz, 1H), 2.92 (dd, J=13.3, 6.5 Hz, 1H), 2.83-2.80 (m, 1H), 2.66 (dd, J=13.4, 7.2 Hz, 1H), 2.61-2.51 (m, 6H), 2.27 (s, 1H), 1.98-1.89 (m, 2H), 1.49-1.41 (m, 1H), 1.23-1.18 (m, 2H). Prep-HPLC conditions: Column: XBridge Prep OBD C18 Column, 30*150 mm, 5 μm; Mobile Phase A: Water (10nmol/L NH₄HCO₃), Mobile Phase B: ACN; Flow rate: 60 mL/min mL/min; Gradient: 14% B to 44% B in 10 min; Wave Length: 254 nm/220 nm; RT1(min): 8.5.

### Synthetic Procedure of V187518

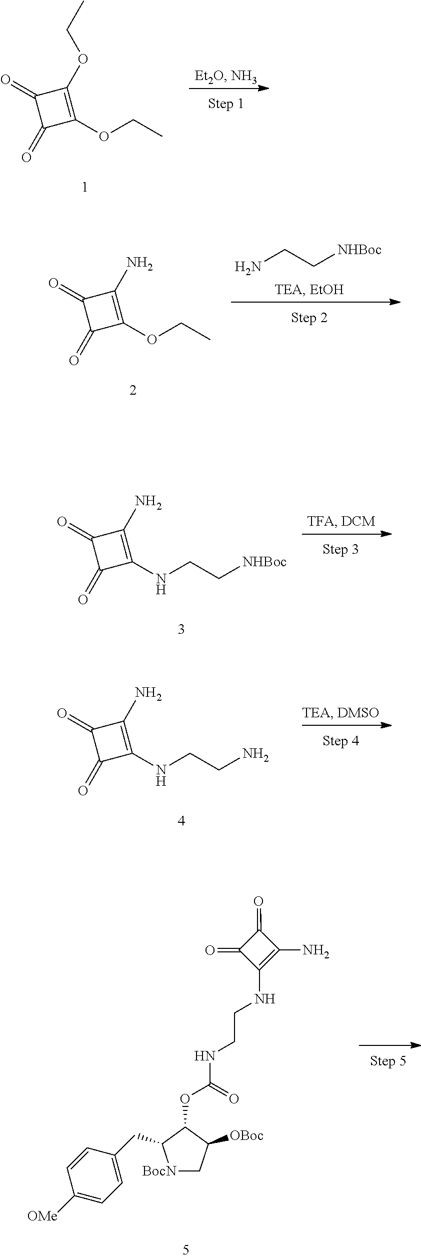

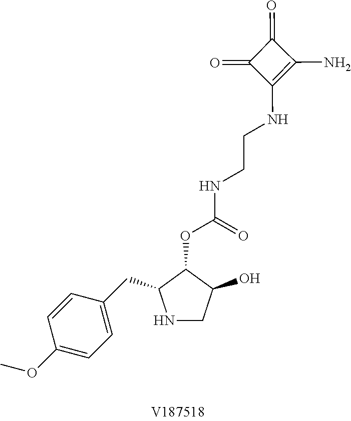 [SMILES: COc1ccc(C[C@H]2NC[C@H](O)[C@H]2OC(=O)NCCNc2c(N)c(=O)c2=O)cc1]

Step 1: To a stirred solution of diethoxycyclobut-3-ene-1,2-dione (1000 mg, 5.87 mmol, 1 eq.) in Et₂O (20 mL) was added NH₃(g) in MeOH (0.05 mL, 5.88 mmol, 1 eq) at room temperature and the resulting mixture was stirred for overnight. Then the mixture was filtered, filter cake was dried in vacuo to obtain 3-amino-4-ethoxycyclobut-3-ene-1,2-dione (700 mg, 84.4%) as a white solid. MS: m/z: Calc'd for C₆H₇NO₃ [M+H]⁺142. found 142.

Step 2: To a stirred solution of 3-amino-4-ethoxycyclobut-3-ene-1,2-dione (200 mg, 1.42 mmol, 1 eq.) and tert-butyl N-(2-aminoethyl)carbamate (454.1 mg, 2.83 mmol, 2 eq.) in EtOH (8 mL) was added TEA (430.2 mg, 4.25 mmol, 3 eq.) and the resulting mixture was stirred at room temperature for overnight. Then the mixture was filtered, filter cake was dried in vacuo to obtain tert-butyl N-{2-[(2-amino-3,4-dioxocyclobut-1-en-1-yl)amino]ethyl}carbamate (150 mg, 41.5%) as a white solid. MS: m/z: Calc'd for C₁₁H₁₇N₃O₄ [M+H−56]⁺200. found 200.

Step 3: To a stirred solution of tert-butyl N-{2-[(2-amino-3,4-dioxocyclobut-1-en-1-yl)amino]ethyl}carbamate (100 mg, 0.39 mmol, 1 eq.) in DCM (5 mL) was added TFA (1 mL) and the resulting mixture was stirred at room temperature for 2 h. Then the mixture was concentrated to obtain 3-amino-4-[(2-aminoethyl)amino]cyclobut-3-ene-1,2-dione (60 mg, 98.7%) as a yellow oil. MS: m/z: Calc'd for C₆H₉N₃O₂[M+H]⁺156. found 156.

Step 4: To a stirred solution of 3-amino-4-[(2-aminoethyl)amino]cyclobut-3-ene-1,2-dione (60 mg, 0.38 mmol, 1 eq.) and tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (227.6 mg, 0.38 mmol, 1 eq.) in DMSO (6 mL) was added TEA (117.4 mg, 1.16 mmol, 3 eq.) at room temperature and the resulting mixture was stirred for overnight. Then the mixture was purified by reversed-phase column to obtain tert-butyl (2R,3S,4S)-3-[({2-[(2-amino-3,4-dioxocyclobut-1-en-1-yl)amino]ethyl}carbamoyl)oxy]-4-[(tert-butoxycarbonyl)oxy]-2-[(4-methoxyphenyl) methyl]pyrrolidine-1-carboxylate (60 mg, 25.7%) as a white solid. MS: m/z: Calc'd for C₂₉H₄₀N₄O₁₀ [M+H−100−56]⁺449. found 449.

Step 5: To a stirred solution of tert-butyl (2R,3S,4S)-3-[({2-[(2-amino-3,4-dioxocyclobut-1-en-1-yl)amino]ethyl}carbamoyl)oxy]-4-[(tert-butoxycarbonyl)oxy]-2-[(4-methoxyphenyl) methyl]pyrrolidine-1-carboxylate (55 mg, 0.09 mmol, 1 eq.) in DCM (5 mL) was added TFA (1 mL) at room temperature and the resulting mixture was stirred for 2 h. Then concentrated and purified by Prep-HPLC to obtain (2R,3S,4S)-4-hydroxy-2-[(4-methoxyphenyl)methyl]pyrrolidin-3-yl N-{2-[(2-amino-3,4-dioxocyclobut-1-en-1-yl)amino]ethyl}carbamate; trifluoroacetic acid (29.3 mg, 62.1%) as a white solid. MS: m/z: Calc'd for C₁₉H₂₄N₄O₆ [M+H]⁺405. found 405. ¹H NMR (400 MHz, Methanol-d₄) δ 7.28-7.17 (m, 2H), 6.97-6.88 (m, 2H), 4.89 (d, J=3.4 Hz, 1H), 4.40 (d, J=4.2 Hz, 1H), 4.13 (dd, J=8.4, 3.4 Hz, 1H), 3.79 (s, 3H), 3.72 (s, 1H), 3.58-3.41 (m, 2H), 3.40-3.34 (m, 2H), 3.33-3.14 (m, 1H), 3.08 (dd, J=14.2, 7.0 Hz, 1H), 2.93 (dd, J=14.1, 8.6 Hz, 1H). Prep-HPLC-conditions: Column: XBridge Shield RP18 OBD Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm; RT1(min): 8.5

### Synthetic Procedure of V187519

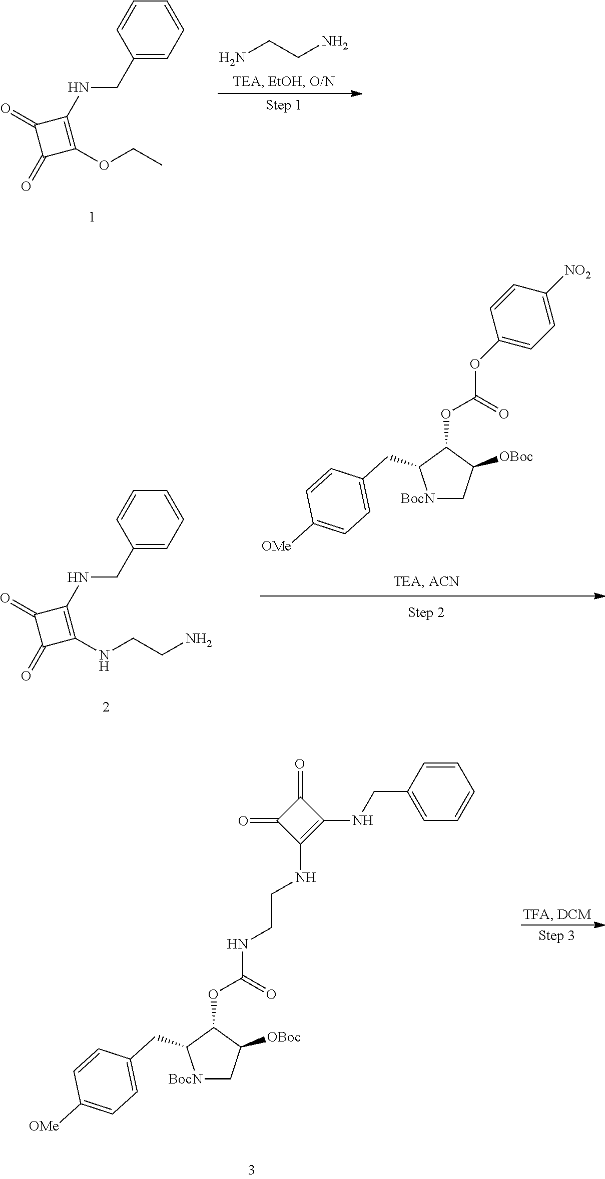

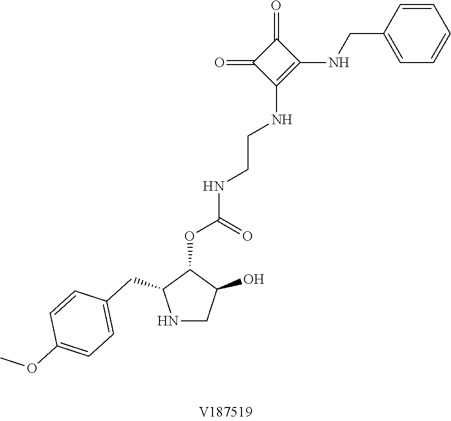 [SMILES: COc1ccc(C[C@H]2NC[C@H](O)[C@H]2OC(=O)NCCNc2c(NCc3ccccc3)c(=O)c2=O)cc1]

Step 1: To a stirred solution of 3-(benzylamino)-4-ethoxycyclobut-3-ene-1,2-dione (230 mg, 0.99 mmol, 1 eq.) and ethylenediamine (119.5 mg, 1.99 mmol, 2 eq.) in EtOH (8 mL) was added TEA (301.9 mg, 2.98 mmol, 3 eq.) at room temperature and the resulting mixture was stirred for overnight. Then the mixture was filtered, washed with EtOH. The filter cake was dried in vacuo to obtain 3-[(2-aminoethyl)amino]-4-(benzylamino)cyclobut-3-ene-1,2-dione (160 mg, 65.6%) as a white solid. MS: m/z: Calc'd for C₁₃H₁₅N₃O₂ [M+H]⁺246. found 246.

Step 2: To a stirred solution of 3-[(2-aminoethyl)amino]-4-(benzylamino)cyclobut-3-ene-1,2-dione (150 mg, 0.62 mmol, 1 eq.) and tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (Int B, 359.9 mg, 0.62 mmol, 1 eq.) in ACN (8 mL) was added TEA (185.6 mg, 1.84 mmol, 3 eq.), and the resulting mixture was stirred at room temperature for overnight. After completion of the reaction monitored by LCMS, the mixture was purified by reverse-phase column to obtain tert-butyl (2R,3S,4S)-3-{[(2-{[2-(benzylamino)-3,4-dioxocyclobut-1-en-1-yl]amino}ethyl) carbamoyl]oxy}-4-[(tert-butoxycarbonyl)oxy]-2-[(4-methoxyphenyl)methyl]pyrrolidine-1-carboxylate (80 mg, 18.83%) as a yellow solid. MS: m/z: Calc'd for C₃₆H₄₆N₄O₁₀ [M+H]⁺695. found 695.

Step 3: To a stirred solution of tert-butyl (2R,3S,4S)-3-{[(2-{[2-(benzylamino)-3,4-dioxocyclobut-1-en-1-yl]amino}ethyl)carbamoyl]oxy}-4-[(tert-butoxycarbonyl)oxy]-2-[(4-methoxyphenyl)methyl]pyrrolidine-1-carboxylate (75 mg, 0.11 mmol, 1 eq.) in DCM (10 mL) was added TFA (2 mL) and the resulting mixture was stirred at room temperature for 2 h. After completion of the reaction monitored by LCMS, the mixture was concentrated. The residue was purified by Prep-HPLC to obtain (2R,3S,4S)-4-hydroxy-2-[(4-methoxyphenyl)methyl]pyrrolidin-3-yl N-(2-{[2-(benzylamino)-3,4-dioxocyclobut-1-en-1-yl]amino}ethyl)carbamate; trifluoroacetic acid (24.9 mg, 37.5%) as a white solid. MS: m/z: Calc'd for C₂₆H₃₀N₄O₆ [M+H]⁺495. found 495. ¹H NMR (400 MHz, Methanol-d₄) δ 7.41-7.35 (m, 1H), 7.35 (s, 3H), 7.35-7.23 (m, 1H), 7.26-7.17 (m, 2H), 6.96-6.88 (m, 2H), 4.90 (d, J=3.4 Hz, 1H), 4.83-4.68 (m, 2H), 4.41 (d, J=4.2 Hz, 1H), 4.11 (dd, J=8.6, 3.4 Hz, 1H), 3.79 (s, 3H), 3.77-3.64 (m, 1H), 3.55 (dd, J=12.7, 4.4 Hz, 1H), 3.47-3.34 (m, 1H), 3.34-3.10 (m, 3H), 3.06 (dd, J=14.2, 6.9 Hz, 1H), 2.92 (dd, J=14.2, 8.7 Hz, 1H). Prep-HPLC-conditions: Column: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 5% B to 35% B in 10 min; Wave Length: 254 nm/220 nm; RT1(min): 8.68

### Synthetic Procedure of V187520

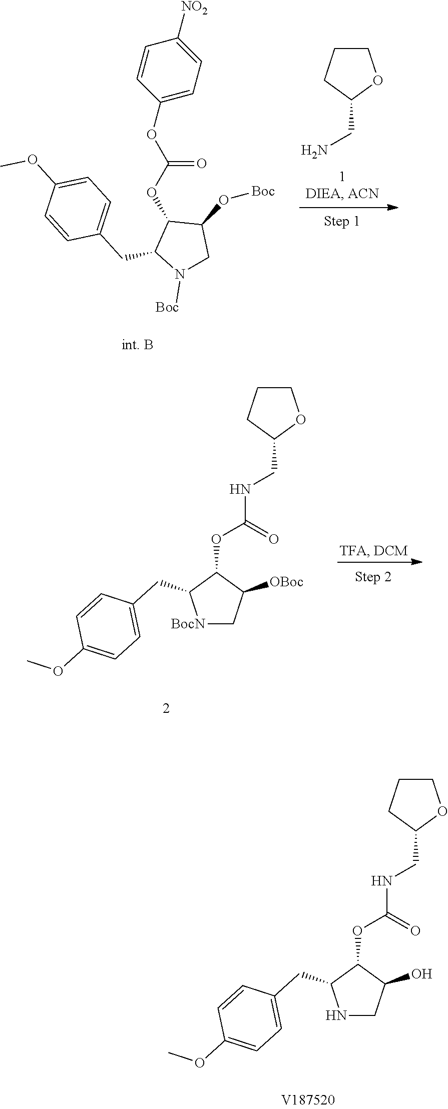

Step 1: To a stirred solution of tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (Int.B, 100 mg, 0.17 mmol, 1 eq.) and 1-[(2S)-oxolan-2-yl]methanamine (34 mg, 0.34 mmol, 2 eq.) in MeCN (3 mL) was added DIEA (88 mg, 0.68 mmol, 4 eq.). The resulting reaction mixture was stirred at ambient temperature for overnight. After completion of the reaction monitored by LCMS, the mixture was purified directly by reversed-phase column chromatography (0.05% TFA in water and acetonitrile) to afford tert-butyl (2R,3S,4S)-4-[(tert-butoxycarbonyl)oxy]-2-[(4-methoxy phenyl)methyl]-3-({[(2S)-oxolan-2-ylmethyl]carbamoyl}oxy)pyrrolidine-1-carboxylate (100 mg, 64.1%) as a yellow solid. C₂₈H₄₂N₂O₉ [M−H]⁻549. Found, 549.

Step 2: To a stirred solution of tert-butyl (2R,3S,4S)-4-[(tert-butoxycarbonyl)oxy]-2-[(4-methoxyphenyl) methyl]-3-({[(2S)-oxolan-2-ylmethyl]carbamoyl}oxy)pyrrolidine-1-carboxylate (100 mg, 0.18 mmol) in DCM (5 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-[(2S)-oxolan-2-ylmethyl]carbamate; trifluoroacetic acid (36.3 mg, 43.0%) as a white solid. MS: m/z: Calc'd for C₁₈H₂₆N₂O₅ [M+H]⁺351. Found, 351. ¹H NMR (400 MHz, Methanol-d₄) δ 7.27-7.19 (m, 2H), 6.96-6.88 (m, 2H), 4.94 (d, J=4.1 Hz, 1H), 4.38 (d, J=4.2 Hz, 1H), 4.19-4.10 (m, 1H), 4.06-3.95 (m, 1H), 3.95-3.85 (m, 1H), 3.79 (s, 3H), 3.83-3.73 (m, 1H), 3.62-3.53 (m, 1H), 3.28 (dd, J=13.9, 4.1 Hz, 1H), 3.25-3.16 (m, 2H), 3.16-3.07 (m, 1H), 3.01-2.91 (m, 1H), 2.09-1.88 (m, 3H), 1.69-1.56 (m, 1H). Prep-HPLC conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 29% B in 10 min; Wave Length: 254 nm/220 nm; RT1(min): 8.68

### Synthetic Procedure of V187521

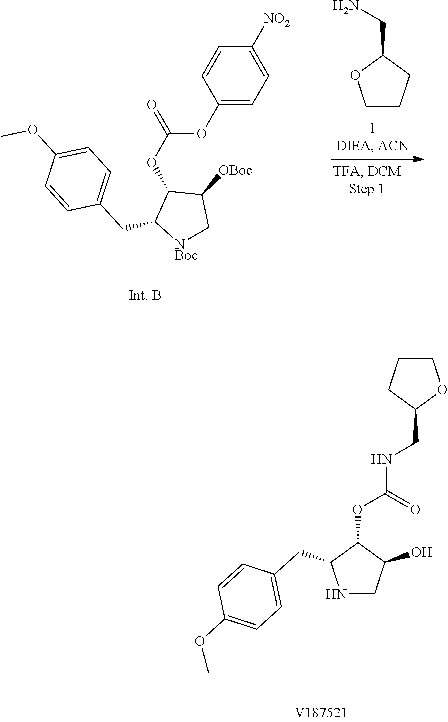

To a stirred solution of tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (Int.B, 100 mg, 0.17 mmol, 1 eq.) and 1-[(2S)-oxolan-2-yl]methanamine (34 mg, 0.34 mmol, 2 eq.) in ACN (3 mL) was added DIEA (66 mg, 0.51 mmol, 3 eq.). The resulting reaction mixture was stirred at ambient temperature for overnight. After completion of the reaction monitored by LCMS, the mixture was concentrated under reduced pressure. To the mixture was added DCM (5 mL) and TFA (5 mL) dropwise at 0° C. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl N-[(2S)-oxolan-2-ylmethyl]carbamate; trifluoroacetic acid (14.0 mg, 17.3%) as a white solid. MS: m/z: Calc'd for C₁₈H₂₆N₂O₅ [M+H]⁺351. found 351. ¹H NMR (400 MHz, DMSO-d₆+D₂O) δ 7.17 (d, J=8.3 Hz, 2H), 6.91-6.83 (m, 2H), 4.68-4.63 (m, 1H), 4.18 (d, J=4.2 Hz, 1H), 3.93 (d, J=3.6 Hz, 1H), 3.74 (dd, J=8.1, 6.5 Hz, 1H), 3.70 (s, 4H), 3.62-3.60 (m, 1H), 3.39 (dd, J=12.8, 4.5 Hz, 1H), 3.22-2.82 (m, 5H), 1.97-1.70 (m, 3H), 1.48-1.40 (m, 1H). Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 30% B in 10 min; Wave Length: 254 nm/220 nm; RT1(min): 8.68.

### Synthetic Procedure of V187534

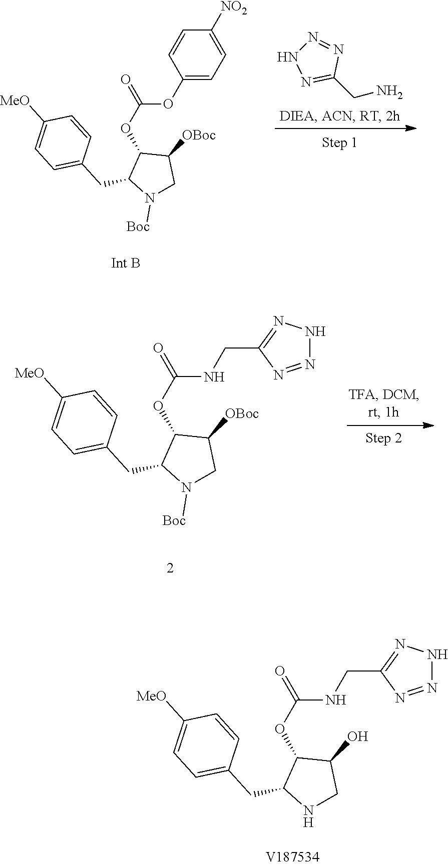

Step 1: To a stirred solution of 1-(2H-1,2,3,4-tetrazol-5-yl)methanamine (34 mg, 0.34 mmol, 2 equiv) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.00 equiv) in ACN (5 mL) was added DIEA (66 mg, 0.51 mmol, 3 equiv). The resulting mixture was stirred for overnight at room temperature. The resulting mixture was concentrated under vacuum and the residue was purified by reversed-phase flash chromatography with the following conditions: column, C18 silica gel; mobile phase, MeCN in Water (0.05% TFA), 10% to 50% gradient in 10 min; detector, UV 254 nm. to afford tert-butyl (2R,3S,4S)-4-[(tert-butoxy carbonyl)oxy]-2-[(4-methoxyphenyl)methyl]-3-{[(2H-1,2,3,4-tetrazol-5-ylmethyl)carbamoyl]oxy}pyrrolidine-1-carboxylate (100 mg, 107.2%) as a yellow oil. MS: m/z: Calc'd for C₂₅H₃₆N₆O₈ [M+H−100−56]⁺393. found 393.

Step 2: To a stirred mixture of tert-butyl (2R,3S,4S)-4-[(tert-butoxycarbonyl)oxy]-2-[(4-methoxy phenyl)methyl]-3-{[(2H-1,2,3,4-tetrazol-5-ylmethyl)carbamoyl]oxy}pyrrolidine-1-carboxylate (100 mg, 0.18 mmol, 1 equiv) in DCM (5 mL) was added TFA (1 mL) at 0° C. The resulting mixture was stirred for 1 h at room temperature. The resulting mixture was concentrated under vacuum and the resulting crude product was purified by Prep-HPLC to afford (2R,3S,4S)-4-hydroxy-2-[(4-methoxyphenyl)methyl]pyrrolidin-3-yl N-(2H-1,2,3,4-tetrazol-5-ylmethyl) carbamate; trifluoroacetic acid (14.4 mg, 16.7%) as a yellow solid. MS: m/z: Calc'd for C₁₅H₂₀N₆O₄ [M+H]⁺349. found 349. ¹H NMR (400 MHz, Methanol-d₄) δ 7.21 (d, J=8.2 Hz, 2H), 6.89 (d, J=8.4 Hz, 2H), 4.96 (d, J=3.5 Hz, 1H), 4.67 (s, 2H), 4.42 (d, J=4.2 Hz, 1H), 4.16-4.03 (m, 1H), 3.79 (s, 3H), 3.68-3.48 (m, 1H), 3.22 (d, J=12.7 Hz, 1H), 3.11 (dd, J=14.2, 7.4 Hz, 1H), 2.98 (dd, J=14.2, 8.5 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 20% B in 10 min; Wave Length: 254 nm/220 nm; RT1(min): 8.68

### Synthetic Procedure of V187536

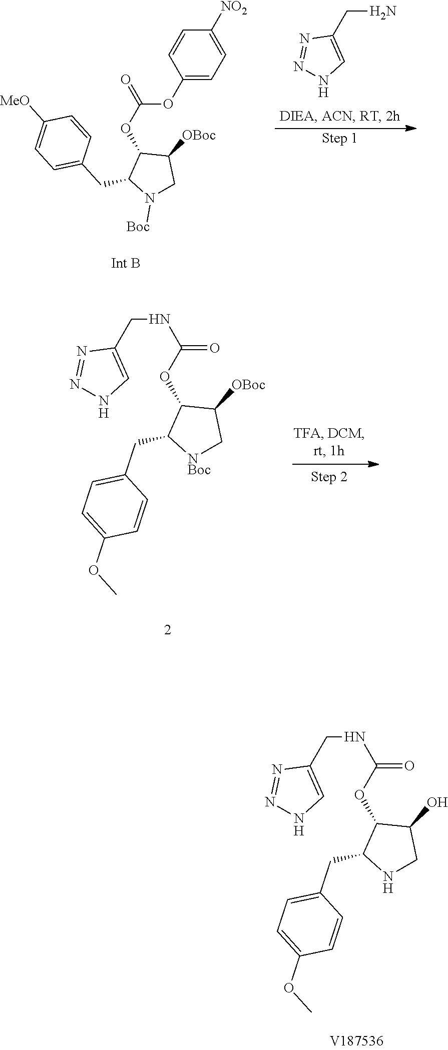

Step 1: To a stirred mixture of 1-(1H-1,2,3-triazol-4-yl)methanamine (33 mg, 0.34 mmol, 2 equiv) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.00 equiv) in ACN (5 mL) was added DIEA (66 mg, 0.51 mmol, 3 equiv). The resulting mixture was stirred for overnight at room temperature. The mixture was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. to afford tert-butyl (2R,3S,4S)-4-[(tert-butoxycarbonyl) oxy]-2-[(4-methoxyphenyl)methyl]-3-{[(1H-1,2,3-triazol-4-ylmethyl)carbamoyl]oxy}pyrrolidine-1-carboxylate (100 mg, 97.4%) as a yellow oil. MS: m/z: Calc'd for C₂₆H₃₇N₅O₈ [M+H−56−56]⁺436. found 436.

Step 2: To a stirred mixture of tert-butyl (2R,3S,4S)-4-[(tert-butoxycarbonyl)oxy]-2-[(4-methoxyphenyl) methyl]-3-{[(1H-1,2,3-triazol-4-ylmethyl)carbamoyl]oxy}pyrrolidine-1-carboxylate (100 mg, 0.18 mmol, 1 equiv) in DCM (5 mL) was added TFA (1 mL) at 0° C. The resulting mixture was stirred for 1 h at room temperature. The resulting mixture was concentrated under vacuum. The crude product (30 mg) was purified by Prep-HPLC to afford (2R,3S,4S)-4-hydroxy-2-[(4-methoxyphenyl)methyl]pyrrolidin-3-yl N-(1H-1,2,3-triazol-4-ylmethyl) carbamate; trifluoroacetic acid (25.1 mg, 29.2%) as a yellow solid. MS: m/z: Calc'd for C₂₆H₃₇N₅O₈ [M+H]⁺348. found 348. ¹H NMR (400 MHz, Methanol-d₄) δ 7.76 (s, 1H), 7.27-7.11 (m, 2H), 6.94-6.85 (m, 2H), 4.94 (s, 1H), 4.57-4.33 (m, 3H), 4.15-4.02 (m, 1H), 3.79 (s, 3H), 3.58 (dd, J=12.6, 4.2 Hz, 1H), 3.22 (d, J=12.6 Hz, 1H), 3.10 (dd, J=14.1, 7.4 Hz, 1H), 3.01-2.91 (m, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 20% B in 10 min; Wave Length: 254 nm/220 nm; RT1(min): 8.68

### Synthetic Procedure of V187537

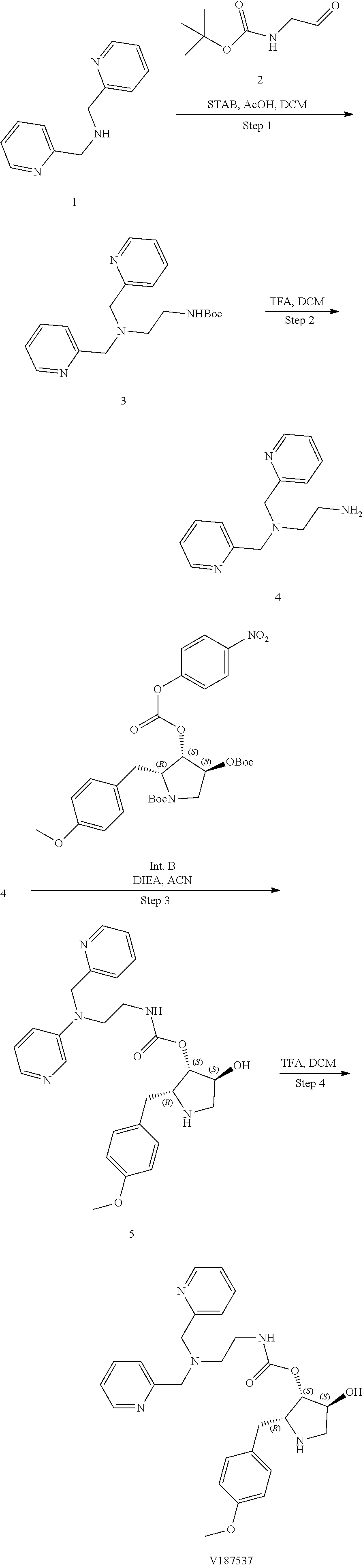

Step 1: To a stirred solution of dipicolylamine (300 mg, 1.51 mmol, 1.0 eq.) and tert-butyl N-(2-oxoethyl)carbamate (359.5 mg, 2.26 mmol, 1.5 eq) in DCM (5 mL) was added AcOH (180.8 mg, 3.01 mmol, 2.0 eq.) at room temperature and the resulting mixture was stirred for 0.5 h. Then NaBH(OAc)₃ (638.2 mg, 3.01 mmol, 2.0 eq.) was added at 0° C. The resulting mixture was stirred at ambient temperature for overnight. The reaction was quenched with Water at 0° C. The aqueous layer was extracted with DCM (2×3 mL). The combined organic layers were washed with brine and dried over anhydrous sodium sulfate. After filtration, the filtrate was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 220 nm. This resulted in tert-butyl N-{2-[bis (pyridin-2-ylmethyl)amino]ethyl}carbamate (300 mg, 58.2%) as a light yellow oil. MS: m/z: Calc'd for C₁₉H₂₆N₄O₂ [M+H]⁺343. Found, 343.

Step 2: To a stirred solution of tert-butyl N-{2-[bis(pyridin-2-ylmethyl)amino]ethyl}carbamate (100 mg, 0.29 mmol, 1.0 eq.) in DCM (1 mL) was added TFA (1 mL) at room temperature. The resulting mixture was stirred at ambient temperature for 1 h. After completion of the reaction monitored by LCMS. The resulting mixture was concentrated under reduced pressure. The crude product was used in the next step directly without further purification. MS: m/z: Calc'd for C₁₄H₁₈N₄ [M+H]⁺243. Found, 243.

Step 3: To a stirred solution of tert-butyl (2R,3S,4S)-4-[(tert-butoxycarbonyl)oxy]-2-[(4-methoxyphenyl) methyl]-3-[(4-nitrophenoxycarbonyl)oxy]pyrrolidine-1-carboxylate (Int.B, 80 mg, 0.21 mmol, 1.0 eq.) and (2-aminoethyl)bis(pyridin-2-ylmethyl)amine (65.87 mg, 0.27 mmol, 2.0 eq.) in MeCN (3 mL) was added DIEA (88 mg, 0.68 mmol, 4 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2S,3S,4R)-3-[({2-[bis(pyridin-2-ylmethyl)amino]ethyl}carbamoyl)oxy]-4-[(tert-butoxycarbonyl)oxy]-2-[(4-methoxyphenyl) methyl]pyrrolidine-1-carboxylate (90 mg, 95.7%) as a light yellow oil. MS: m/z: Calc'd for C₂₇H₃₃N₅O₄ [M+H]⁺692. Found, 692.

Step 4: To a stirred solution of tert-butyl (2S,3S,4R)-3-[({2-[bis(pyridin-2-ylmethyl) amino]ethyl}carbamoyl)oxy]-4-[(tert-butoxycarbonyl)oxy]-2-[(4-methoxyphenyl) methyl]pyrrolidine-1-carboxylate (60 mg, 0.09 mmol, 1.0 eq.) in DCM (3 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2S,3S,4R)-4-hydroxy-2-[(4-methoxyphenyl) methyl]pyrrolidin-3-yl N-{2-[bis(pyridin-2-ylmethyl)amino]ethyl}carbamate; trifluoroacetic acid (26.0 mg, 48.8%) as a yellow semi-solid. MS: m/z: Calc'd for C₂₇H₃₃N₅O₄ [M+H]⁺492. Found, 492. ¹H NMR (400 MHz, Methanol-d₄) δ 8.74-8.65 (m, 2H), 8.07-8.03 (m, 2H), 7.64 (d, J=7.8 Hz, 2H), 7.58-7.54 (m, 2H), 7.28-7.19 (m, 2H), 6.98-6.87 (m, 2H), 4.95 (d, J=3.6 Hz, 1H), 4.50 (s, 4H), 4.39 (d, J=4.2 Hz, 1H), 4.20-4.16 (m, 1H), 3.78 (s, 3H), 3.62-3.50 (m, 3H), 3.26-3.21 (m, 3H), 3.10 (dd, J=14.2, 6.9 Hz, 1H), 2.97 (dd, J=14.2, 8.6 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 20% B in 10 min; Wave Length: 254 nm/220 nm; RT1(min): 8.68

### Synthetic Procedure of V187538

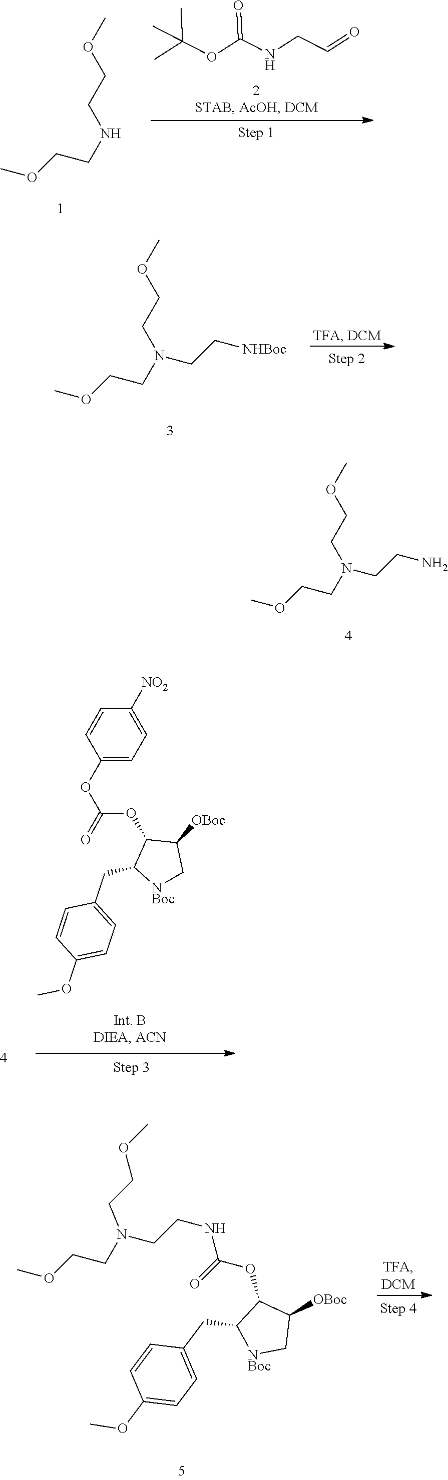

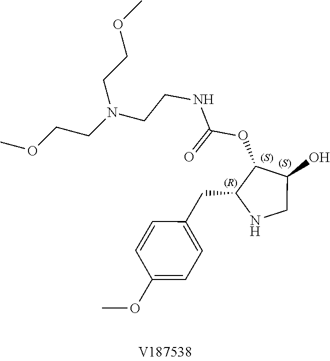

Step 1: To a stirred solution of bis(2-methoxyethyl)amine (300 mg, 2.25 mmol, 1.0 eq.) and tert-butyl N-(2-oxoethyl)carbamate (537.8 mg, 3.38 mmol, 1.5 eq.) in DCM (5 mL) was added AcOH (270.5 mg, 4.51 mmol, 2.0 eq.) at room temperature stirred 0.5 h. Then added NaBH(OAc)₃ (954.8 mg, 4.511 mmol, 2.0 eq.) at 0° C. The resulting mixture was stirred at ambient temperature for overnight. The reaction was quenched with Water at 0° C. The aqueous layer was extracted with DCM (2×3 mL). The combined organic layers were washed with brine and dried over anhydrous sodium sulfate. After filtration, the filtrate was concentrated under reduced pressure. The resulting residue was purified by reversed-phase flash chromatography with the following conditions: column, C18 silica gel; mobile phase, MeCN in Water (0.1% TFA), 10% to 50% gradient in 10 min; detector, UV 220 nm. This resulted in tert-butyl N-{2-[bis(2-methoxyethyl) amino]ethyl}carbamate (100 mg, 16.0%) as a colorless oil. MS: m/z: Calc'd for C₁₃H₂₈N₂O₄ [M+H]⁺277. Found, 277.

Step 2: To a stirred solution of tert-butyl N-{2-[bis(2-methoxyethyl)amino]ethyl}carbamate (100 mg, 0.36 mmol) in DCM (2 mL) was added TFA (1 mL) at room temperature. The resulting mixture was stirred at ambient temperature for 1 h. After completion of the reaction monitored by LCMS. The resulting mixture was concentrated under reduced pressure. The crude product was used in the next step directly without further purification. MS: m/z: Calc'd for C₈H₂₀N₂O₂ [M+H]⁺177. Found, 177.

Step 3: To a stirred solution of tert-butyl (2R,3S,4S)-4-[(tert-butoxycarbonyl)oxy]-2-[(4-methoxyphenyl) methyl]-3-[(4-nitrophenoxycarbonyl)oxy]pyrrolidine-1-carboxylate (Int.B, 80 mg, 0.21 mmol, 1.0 eq.) and (2-aminoethyl)bis(2-methoxyethyl)amine (72.6 mg, 0.41 mmol, 2.0 eq.) in MeCN (3 mL) was added DIEA (88 mg, 0.68 mmol, 4 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford (2S,3S,4R)-4-hydroxy-2-[(4-methoxyphenyl) methyl]pyrrolidin-3-yl N-{2-[bis(2-methoxyethyl)amino]ethyl}carbamate (70 mg, 79.9%) as a light yellow oil. MS: m/z: Calc'd for C₃₁H₅₁N₃O₁₀ [M+H]⁺626. Found, 626.

Step 4: To a stirred solution of tert-butyl (2S,3S,4R)-3-[({2-[bis(2-methoxyethyl)amino]ethyl}carbamoyl)oxy]-4-[(tert-butoxycarbonyl)oxy]-2-[(4-methoxyphenyl)methyl]pyrrolidine-1-carboxylate (60 mg, 0.10 mmol, 1.0 eq.) in DCM (3 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2S,3S,4R)-4-hydroxy-2-[(4-methoxyphenyl)methyl]pyrrolidin-3-yl N-{2-[bis(2-methoxyethyl)amino]ethyl}carbamate; trifluoroacetic acid (28.7 mg, 55.2%) as a light yellow semi-solid. MS: m/z: Calc'd for C₂₁H₃₅N₃O₆ [M+H]⁺426. Found, 426. ¹H NMR (400 MHz, Methanol-d₄) δ 7.28-7.22 (m, 2H), 6.97-6.91 (m, 2H), 5.03-4.99 (m, 1H), 4.43-4.39 (m, 1H), 4.22-4.88 (m, 1H), 3.80 (s, 3H), 3.77-3.73 (m, 4H), 3.62-3.55 (m, 3H), 3.55-3.51 (m, 4H), 3.42 (s, 8H), 3.23 (d, J=12.6 Hz, 1H), 3.12 (dd, J=14.3, 6.9 Hz, 1H), 2.99 (dd, J=14.2, 8.7 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 20% B in 10 min; Wave Length: 254 nm/220 nm; RT1(min): 8.68

### Synthetic Procedure of V187539

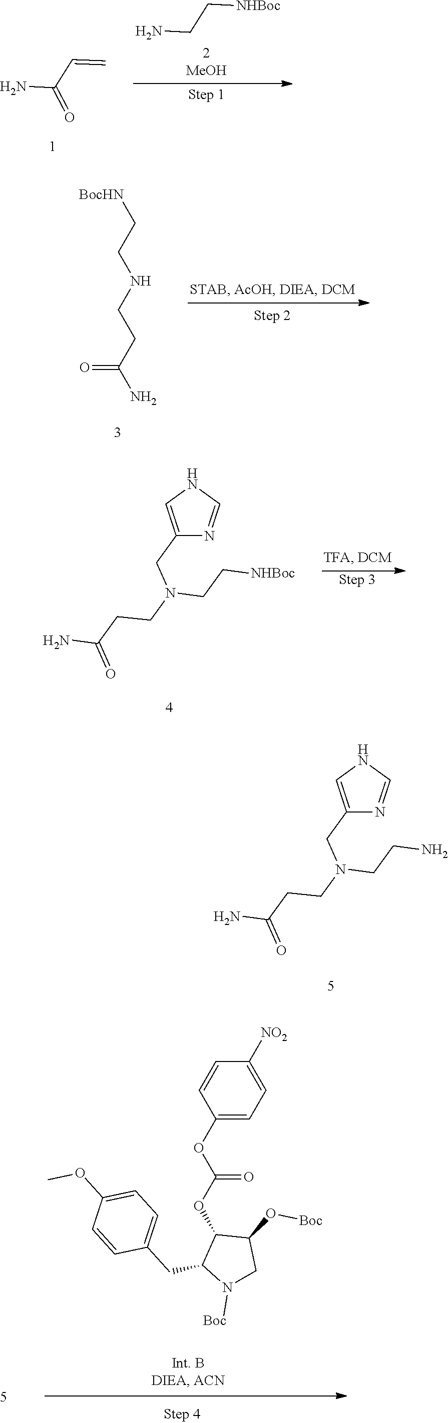

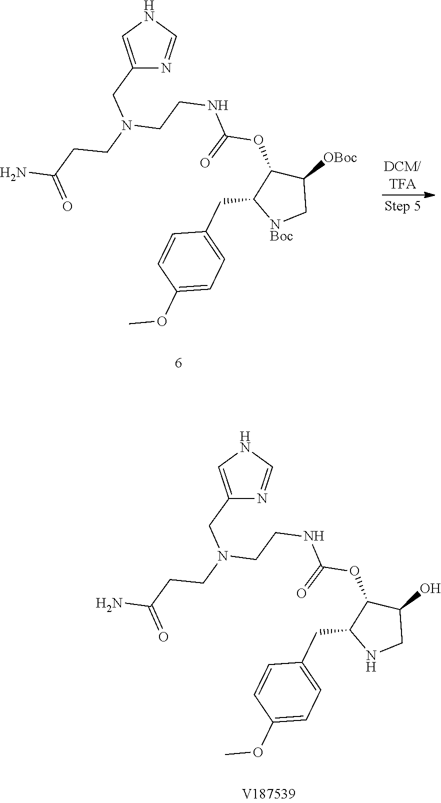

Step 1: To a stirred solution of polyacrylamide (100 mg, 1.40 mmol, 1 eq) in MeOH (5 mL) was added tert-butyl N-(2-aminoethyl)carbamate (225 mg, 1.41 mmol, 1 eq). The resulting reaction mixture was stirred at 60° C. for overnight. After completion of the reaction monitored by LCMS. The mixture was purified directly by reversed-phase column chromatography (10 mmol/L NH₄HCO₃ in water and acetonitrile) to afford tert-butyl N-{2-[(2-carbamoylethyl)amino]ethyl}carbamate (325 mg, 99.9%) as a colorless oil. MS: m/z: Calc'd for C₁₀H₂₁N₃O₃[M+H]⁺232. Found, 232.

Step 2: To a stirred solution of tert-butyl N-{2-[(2-carbamoylethyl)amino]ethyl}carbamate (295 mg, 1.27 mmol, 1 eq) and 1H-imidazole-4-carbaldehyde (122 mg, 1.27 mmol, 1 eq) in MeOH (10 mL) was added AcOH (306 mg, 5.10 mmol, 4 eq) dropwise at room temperature. The resulting mixture was stirred at room temperature for 12 h. The mixture was added NaBH₃CN (541 mg, 2.55 mmol, 2 eq). The resulting mixture was stirred at room temperature for 5 h. After completion of the reaction monitored by LCMS. The mixture was concentrated under reduced pressure. The residue was purified directly by reversed-phase column chromatography (10 mmol/L NH₄HCO₃ in water and acetonitrile) to afford tert-butyl N-{2-[(2-carbamoylethyl) (1H-imidazol-4-ylmethyl)amino]ethyl}carbamate (200 mg, 50.36%) as a colorless oil. MS: m/z: Calc'd for C₁₄H₂₅N₅O₃ [M+H]⁺312. Found, 312.

Step 3: To a stirred solution of tert-butyl tert-butyl N-{2-[(2-carbamoylethyl)(1H-imidazol-4-ylmethyl)amino]ethyl}carbamate (295 mg, 0.94 mmol) in DCM (10 mL) was added TFA (10 mL) dropwise at 0° C. The resulting mixture was stirred at room temperature for 1 h. After completion of the reaction monitored by LCMS, the resulting mixture was concentrated under reduced pressure, the residue was purified by reversed-phase column chromatography (10 mmol/L NH₄HCO₃ in water and acetonitrile) to afford 3-[(2-aminoethyl)(1H-imidazol-4-ylmethyl)amino]propanamide (200 mg, 99.9%) as a colorless oil. MS: m/z: Calc'd for C₉H₁₇N₅O [M+H]⁺212. Found, 212.

Step 4: To a stirred solution of tert-butyl (2R,3S,4S)-4-[(tert-butoxycarbonyl)oxy]-2-[(4-methoxyphenyl) methyl]-3-[(4-nitrophenoxycarbonyl)oxy]pyrrolidine-1-carboxylate (Int.B, 100 mg, 0.17 mmol, 1 eq.) and 3-[(2-aminoethyl)(1H-imidazol-4-ylmethyl)amino]propanamide (72 mg, 0.34 mmol, 2 eq.) in ACN (5 mL) were added DIEA (66 mg, 0.51 mmol, 3 eq.) at r.t. The resulting mixture was stirred at 60° C. for 2 h. After completion of the reaction monitored by LCMS, the resulting mixture was purified directly by reversed-phase column chromatography (10 mmol/L NH₄HCO₃ in water and acetonitrile) to afford tert-butyl (2S,3S,4R)-4-[(tert-butoxycarbonyl)oxy]-3-[({2-[(2-carbamoylethyl)(1H-imidazol-4-ylmethyl)amino]ethyl}carbamoyl)oxy]-2-[(4-methoxyphenyl) methyl]pyrrolidine-1-carboxylate (80 mg, 71.26%) as a colorless oil. MS: m/z: Calc'd for C₃₂H₄₈N₆O₉ [M+H]⁺661. Found, 661.

Step 5: To a stirred solution of tert-butyl (2S,3S,4R)-4-[(tert-butoxycarbonyl)oxy]-3-[({2-[(2-carbamoylethyl)(1H-imidazol-4-ylmethyl)amino]ethyl}carbamoyl)oxy]-2-[(4-methoxyphenyl) methyl]pyrrolidine-1-carboxylate (80 mg, 0.12 mmol) in DCM (5 mL) was added TFA (5 mL) at room temperature. The resulting mixture was stirred at room temperature for 1 h. After completion of the reaction monitored by LCMS, the mixture was concentrated. The residue was purified by Prep-HPLC to afford (2S,3S,4R)-4-hydroxy-2-[(4-methoxyphenyl)methyl]pyrrolidin-3-yl N-{2-[(2-carbamoylethyl)(1H-imidazol-4-ylmethyl)amino]ethyl}carbamate; trifluoroacetic acid (17.9 mg, 25.3%) as a white solid. MS: m/z: Calc'd for C₂₂H₃₂N₆O₅ [M+H]⁺461. Found, 461. ¹H NMR (400 MHz, DMSO-d6) δ 7.55 (s, 1H), 7.38 (s, 1H), 7.25 (s, 1H), 7.08 (d, J=8.2 Hz, 2H), 6.89 (s, 1H), 6.78 (dd, J=11.0, 5.6 Hz, 3H), 4.54-4.47 (m, 1H), 4.04 (s, 1H), 3.93 (s, 1H), 3.70 (s, 3H), 3.57 (s, 2H), 3.31 (s, 1H), 3.25 (s, 1H), 3.10-3.08 (m, 3H), 2.64 (t, J=7.2 Hz, 3H), 2.54 (s, 1H), 2.51 (d, J=1.8 Hz, 1H), 2.50-2.43 (m, 3H), 2.23 (t, J=7.2 Hz, 2H). Prep-HPLC conditions: Column: XBridge Prep OBD C18 Column, 30*150 mm, 5 μm; Mobile Phase A: Water (10 nmol/L NH₄HCO₃), Mobile Phase B: ACN; Flow rate: 60 mL/min mL/min; Gradient: 2% B to 26% B in 13 min; Wave Length: 254 nm/220 nm; RT1(min): 8.87.

### Synthetic Procedure of V187540

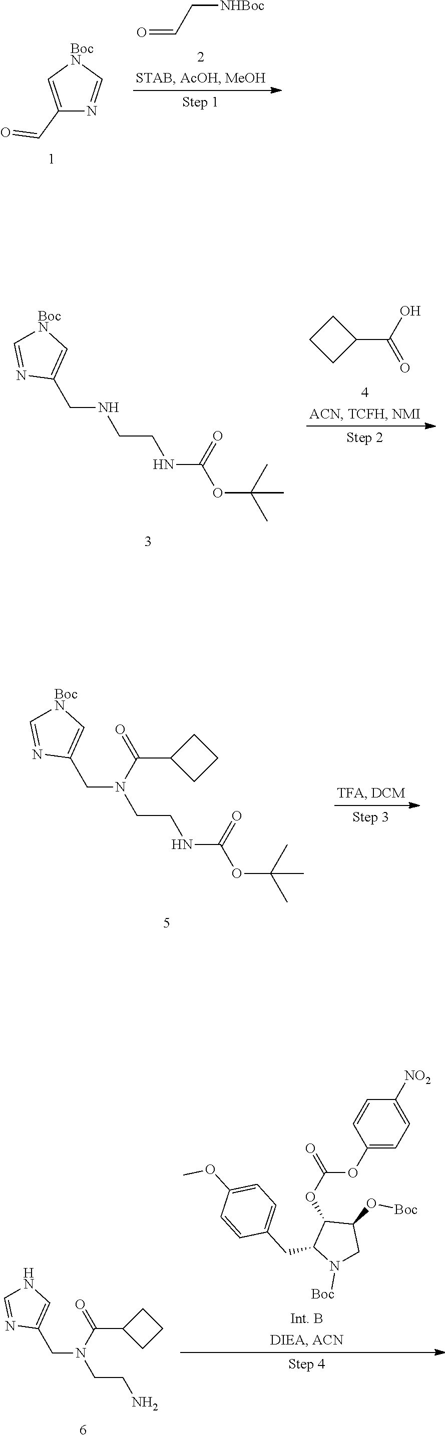

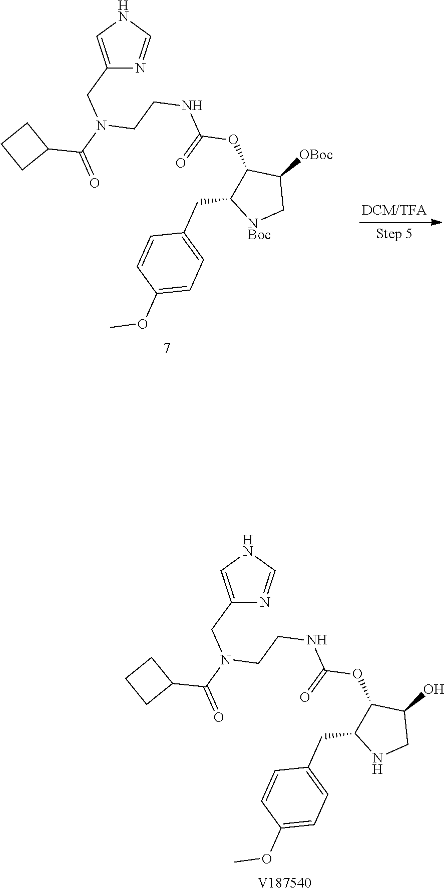

Step 1: To a stirred solution of tert-butyl 4-formylimidazole-1-carboxylate (200 mg, 1.02 mmol, 1 eq) and tert-butyl N-(2-aminoethyl)carbamate (327 mg, 2.04 mmol, 2 eq.) in MeOH (15 mL) was added AcOH (122 mg, 2.04 mmol, 2 eq.) at r.t. The resulting reaction mixture was stirred at room temperature for overnight. STAB (432 mg, 2.04 mmol, 2 eq.) was added to the mixture. The resulting mixture was stirred at room temperature for 1 h. After completion of the reaction monitored by LCMS, the resulting mixture was concentrated under reduced pressure. The residue was purified by reversed-phase column chromatography (10 mmol/L NH₄HCO₃ in water and acetonitrile) to afford tert-butyl 4-[({2-[(tert-butoxycarbonyl)amino]ethyl}amino)methyl]imidazole-1-carboxylate (136 mg, 39.2%) as a colorless oil. MS: m/z: Calc'd for C₁₆H₂₈N₄O₄ [M+H]⁺341. Found, 341.

Step 2: To a stirred solution of tert-butyl 4-[({2-[(tert-butoxycarbonyl)amino]ethyl}amino) methyl]imidazole-1-carboxylate (125 mg, 0.37 mmol, 1 eq.) and cyclobutanecarboxylic acid (44 mg, 0.44 mmol, 1.2 eq.) in ACN (6 mL) were added NMI (90 mg, 1.10 mmol, 3 eq.) and TCFH (206 mg, 0.73 mmol, 2 eq.) at room temperature. The resulting mixture was stirred at room temperature for 2 h. After completion of the reaction monitored by LCMS, the resulting mixture was concentrated under reduced pressure. The residue was purified directly by Prep-TLC (EA as eluent) to afford tert-butyl 4-[(N-{2-[(tert-butoxycarbonyl) amino]ethyl}-1-cyclobutyl formamido) methyl]imidazole-1-carboxylate (105 mg, 67.7%) as a light yellow oil. MS: m/z: Calc'd for C₂₁H₃₄N₄O₅ [M+H−100]⁺323. Found, 323.

Step 3: To a stirred solution of tert-butyl 4-[(N-{2-[(tert-butoxycarbonyl)amino]ethyl}-1-cyclobutylformamido)methyl]imidazole-1-carboxylate (100 mg, 0.23 mmol) in DCM (5 mL) was added TFA (5 mL) dropwise at 0° C. The resulting mixture was stirred for 1 h at room temperature. After completion of the reaction monitored by LCMS, the resulting mixture was concentrated under reduced pressure to afford crude product which was used directly in the next step without further purification. MS: m/z: Calc'd for C₁₁H₁₈N₄O [M+H]⁺223. Found, 223.

Step 4: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq.) and N-(2-aminoethyl)-N-(1H-imidazol-4-ylmethyl)cyclobutene carboxamide (76 mg, 0.34 mmol, 2 eq.) in ACN (5 mL) was added DIEA (110 mg, 0.85 mmol, 5 eq.). The resulting mixture was stirred at room temperature for 2 h. After completion of the reaction monitored by LCMS, the resulting mixture was purified directly by reversed-phase column chromatography (10 mmol/L NH₄HCO₃ in water and acetonitrile) to afford tert-butyl (2S,3S,4R)-4-[(tert-butoxycarbonyl)oxy]-3-[({2-[l-cyclobutyl-N-(1H-imidazol-4-ylmethyl) formamido]ethyl}carbamoyl) oxy]-2-[(4-methoxyphenyl)methyl]pyrrolidine-1-carboxylate (59 mg, 51.7%) as a white solid. MS: m/z: Calc'd for C₃₄H₄₉N₅O₉ [M+H]⁺672. Found, 672.

Step 5: To a stirred solution of tert-butyl (2S,3S,4R)-4-[(tert-butoxycarbonyl)oxy]-3-[({2-[1-cyclobutyl-N-(1H-imidazol-4-ylmethyl)formamido]ethyl}carbamoyl)oxy]-2-[(4-methoxy phenyl)methyl]pyrrolidine-1-carboxylate (54 mg, 0.08 mmol) in DCM (5 mL) was added TFA (5 mL) at room temperature. The resulting mixture was stirred at room temperature for 1 h. After completion of the reaction monitored by LCMS, the mixture was concentrated. The residue was purified by Prep-HPLC to afford (2S,3S,4R)-4-hydroxy-2-[(4-methoxyphenyl) methyl]pyrrolidin-3-yl N-{2-[1-cyclobutyl-N-(1H-imidazol-4-ylmethyl)formamido]ethyl}carbamate (7.7 mg, 19.9%) as a white solid. MS: m/z: Calc'd for C₂₄H₃₃N₅O₅ [M+H]⁺472. Found, 472. ¹H NMR (400 MHz, Methanol-d4) δ 7.65-7.62 (m, 1H), 7.13 (dd, J=8.7, 3.3 Hz, 2H), 7.01 (d, J=8.7 Hz, 1H), 6.83 (dd, J=8.6, 3.2 Hz, 2H), 4.63 (dd, J=7.2, 3.7 Hz, 1H), 4.56 (s, 1H), 4.47 (s, 1H), 4.19-4.10 (m, 1H), 3.76 (d, J=1.9 Hz, 3H), 3.65-3.46 (m, 3H), 3.38 (d, J=6.3 Hz, 1H), 3.28-4.24 (m, 3H), 2.87-2.67 (m, 3H), 2.40-2.25 (m, 2H), 2.18-2.15 (m, 2H), 1.99-1.96 (m, 1H), 1.85-1.83 (m, 1H). Prep-HPLC conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min mL/min; Gradient: 11% B to 36% B in 13 min; Wave Length: 254 nm/220 nm; RT1(min): 7.57; 9.60.

### Synthetic Procedure of V187551

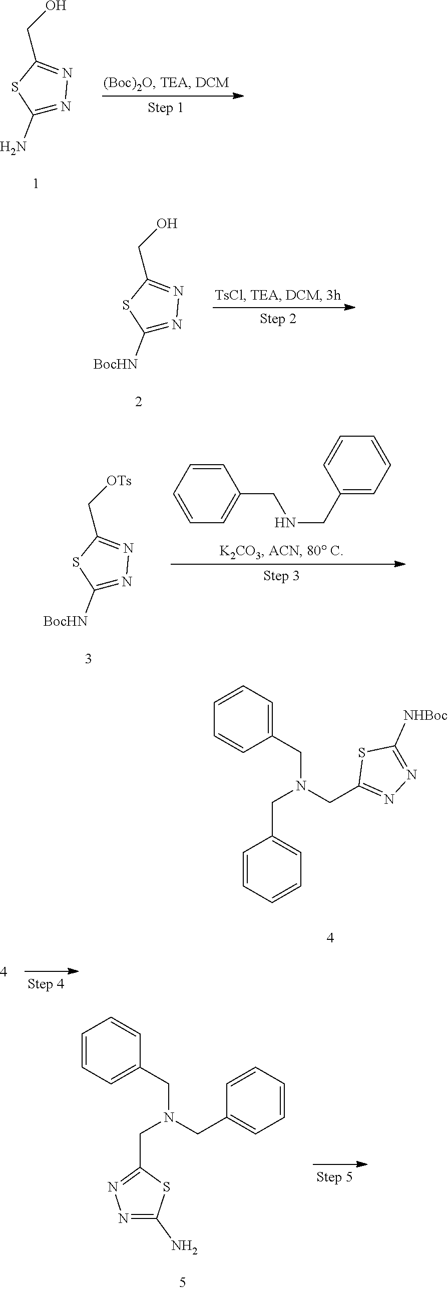 [SMILES: Nc1nnc(CO)s1 ; CC(C)(C)OC(=O)Nc1nnc(CO)s1 ; Cc1ccc(S(=O)(=O)OCc2nnc(NC(=O)OC(C)(C)C)s2)cc1 ; c1ccc(CNCc2ccccc2)cc1 ; CC(C)(C)OC(=O)Nc1nnc(CN(Cc2ccccc2)Cc2ccccc2)s1 ; Nc1nnc(CN(Cc2ccccc2)Cc2ccccc2)s1]

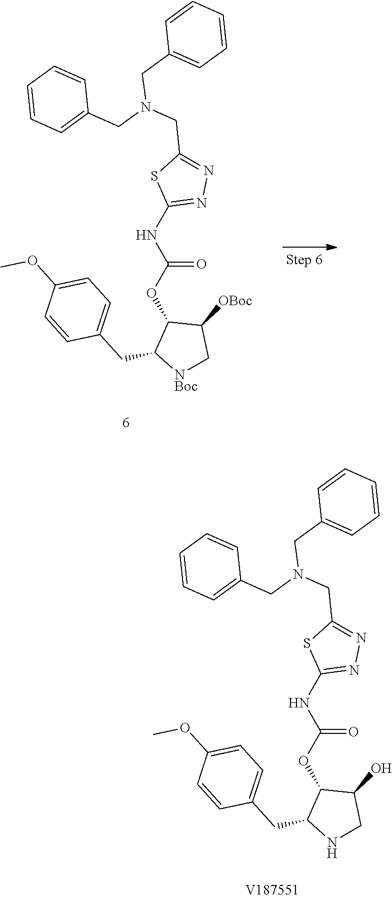

Step 1: To a stirred solution of (5-amino-1,3,4-thiadiazol-2-yl)methanol (500 mg, 3.81 mmol, 1 equiv) and di-tert-butyl dicarbonate (1664 mg, 7.62 mmol, 2 equiv) in DCM (100 mL) was added TEA (1157.3 mg, 11.43 mmol, 3 equiv) at 0° C. under N2 atmosphere. The resulting mixture was stirred for overnight at room temperature. Desired product could be detected by LCMS. The resulting mixture was concentrated under vacuum. The residue was purified by silica gel column chromatography, eluted with PE/EA (1:9) to afford tert-butyl N-[5-(hydroxymethyl)-1,3,4-thiadiazol-2-yl]carbamate (150 mg, 17.0%) as a white solid. MS: m/z: Calc'd for C₈H₁₃N₃O₃S [M+H]⁺232. found 232.

Step 2: To a stirred mixture of tert-butyl N-[5-(hydroxymethyl)-1,3,4-thiadiazol-2-yl]carbamate (200 mg, 0.86 mmol, 1 equiv) and Et₃N (262 mg, 2.59 mmol, 3 equiv) in DCM (1 mL) was added TsCl (197.8 mg, 1.03 mmol, 1.2 equiv) at 0° C. under N2 atmosphere. The resulting mixture stirred for overnight at room temperature. The resulting mixture was quenched with water and extracted with EtOAc (2×100 mL). The combined organic layers were washed with water (2×20 mL), dried over anhydrous Na₂SO₄. After filtration, the filtrate was concentrated under reduced pressure. This resulted in tert-butyl N-(5-{[(4-methylbenzene sulfonyl)oxy]methyl}-1,3,4-thiadiazol-2-yl)carbamate (250 mg, 75.0%) as a yellow solid. MS: m/z: Calc'd for C₁₅H₁₉N₃O₅S₂[M+H−56]⁺330. found 330.

Step 3: To a stirred mixture of tert-butyl N-(5-{[(4-methylbenzenesulfonyl)oxy]methyl}-1,3,4-thiadiazol-2-yl)carbamate (500 mg, 1.29 mmol, 1 equiv) and dibenzyl amine (512 mg, 2.59 mmol, 2 equiv) in DMF (10 mL) was added K₂CO₃ (538 mg, 3.89 mmol, 3 equiv) at 0° C. The resulting mixture was stirred for 1 h at room temperature. Desired product could be detected by LCMS. The resulting mixture was quenched with water and extracted with EtOAc (2×200 mL). The combined organic layers were washed with water (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in tert-butyl N-{5-[(dibenzylamino)methyl]-1,3,4-thiadiazol-2-yl}carbamate as a yellow oil. MS: m/z: Calc'd for C₂₂H₂₆N₄O₂S [M+H]⁺411. found 411.

Step 4: To a stirred mixture of tert-butyl N-{5-[(dibenzylamino)methyl]-1,3,4-thiadiazol-2-yl}carbamate (120 mg, 0.29 mmol, 1 equiv) in DCM (5 mL) was added TFA (1 mL). The resulting mixture was stirred for 2 hours at room temperature and the mixture was concentrated under vacuum. This resulted in 5-[(dibenzylamino)methyl]-1,3,4-thiadiazol-2-amine (100 mg, 93.6%) as a yellow oil. MS: m/z: Calc'd for C₁₇H₁₈N₄S [M+H]⁺311. found 311.

Step 5: To a stirred mixture of 5-[(dibenzylamino)methyl]-1,3,4-thiadiazol-2-amine (105 mg, 0.34 mmol, 2 equiv) and tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (100 mg, 0.17 mmol, 1.00 equiv) in ACN (5 mL) was added DIEA (66 mg, 0.51 mmol, 3 equiv). The resulting mixture was stirred for 72 h at room temperature. The resulting mixture was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in tert-butyl (2R,3S,4S)-4-[(tert-butoxycarbonyl)oxy]-3-[({5-[(dibenzylamino)methyl]-1,3,4-thiadiazol-2-yl}carbamoyl)oxy]-2-[(4-methoxyphenyl)methyl]pyrrolidine-1-carboxylate (110 mg, 85.2%) as a white solid. MS: m/z: Calc'd for C₄₀H₄₉N₅O₈S [M+H]⁺760. found 760.

Step 6: To a stirred mixture of tert-butyl (2R,3S,4S)-4-[(tert-butoxycarbonyl)oxy]-3-[({5-[(dibenzylamino)methyl]-1,3,4-thiadiazol-2-yl}carbamoyl)oxy]-2-[(4-methoxyphenyl) methyl]pyrrolidine-1-carboxylate (100 mg, 0.13 mmol, 1 equiv) in DCM (5 mL) as added TFA (1 mL) at 0° C. The resulting mixture was stirred for 2 h at room temperature. Desired product could be detected by LCMS. The resulting mixture was concentrated under reduced pressure. The crude product (50 mg) was purified by Prep-HPLC to afford (2R,3S,4S)-4-hydroxy-2-[(4-methoxyphenyl)methyl]pyrrolidin-3-yl N-{5-[(dibenzylamino)methyl]-1,3,4-thiadiazol-2-yl}carbamate (31.2 mg, 35.0%) as a white solid. MS: m/z: Calc'd for C₃₀H₃₃N₅O₄S [M+H]⁺560. found 560. ¹H NMR (400 MHz, Methanol-d₄) δ 7.53-7.20 (m, 12H), 6.95-6.89 (m, 2H), 5.17 (d, J=3.9 Hz, 1H), 4.54 (d, J=4.2 Hz, 1H), 4.31-4.19 (m, 1H), 4.07 (s, 2H), 3.81 (d, J=38.5 Hz, 7H), 3.68-3.58 (m, 1H), 3.28 (d, J=12.7 Hz, 1H), 3.18 (dd, J=14.4, 7.1 Hz, 1H), 3.04-2.98 (m, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 19% B to 49% B in 10 min; Wave Length: 254 nm/220 nm; RT1(min): 8.68

### Synthetic Procedure of V187552

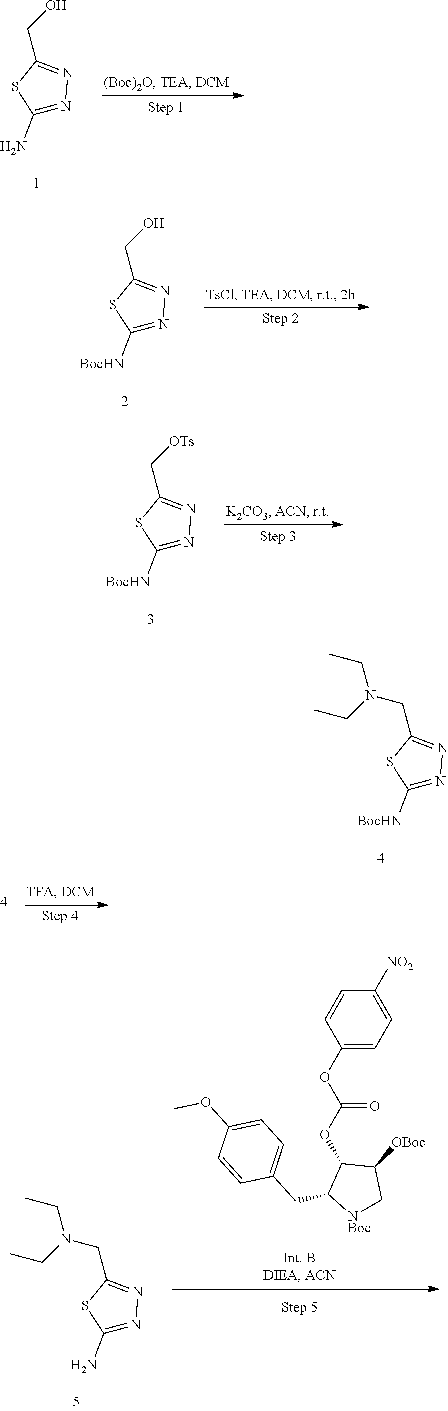

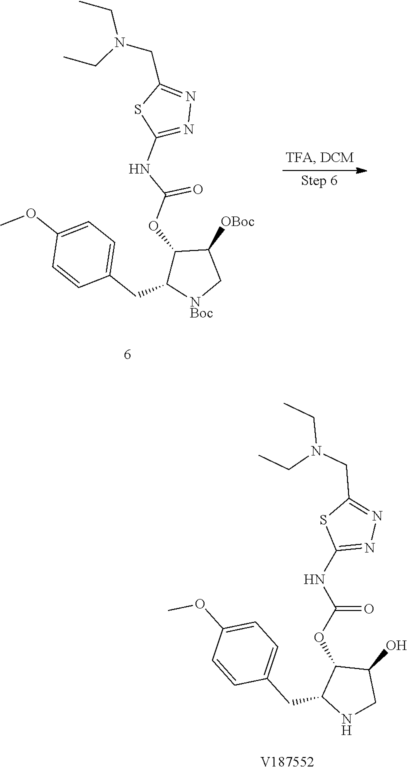

Step 1: To a stirred solution of (5-amino-1,3,4-thiadiazol-2-yl)methanol (500 mg, 3.81 mmol, 1 equiv) and di-tert-butyl dicarbonate (1664 mg, 7.62 mmol, 2 equiv) in DCM (100 mL) was added TEA (1157 mg, 11.43 mmol, 3 equiv) at 0° C. The resulting mixture was stirred for overnight at room temperature. The mixture was concentrated under vacuum. The residue was purified by silica gel column chromatography, eluted with PE/EA (1:9) to afford tert-butyl N-[5-(hydroxymethyl)-1,3,4-thiadiazol-2-yl]carbamate (150 mg, 17.0%) as a white solid. MS: m/z: Calc'd for C₈H₁₃N₃O₃S [M+H]⁺232. found 232.

Step 2: To a stirred mixture of tert-butyl N-[5-(hydroxymethyl)-1,3,4-thiadiazol-2-yl]carbamate (200 mg, 0.86 mmol, 1 equiv) and Et₃N (263 mg, 2.59 mmol, 3 equiv) in DCM (1 mL) was added TsCl (198 mg, 1.03 mmol, 1.2 equiv) at 0° C. The resulting mixture was stirred for overnight at room temperature. The mixture was extracted with EtOAc (2×100 mL). The combined organic layers were washed with water (2×20 mL), dried over anhydrous Na₂SO₄. After filtration, the filtrate was concentrated under reduced pressure. This resulted in tert-butyl N-(5-{[(4-methylbenzenesulfonyl)oxy]methyl}-1,3,4-thiadiazol-2-yl)carbamate (250 mg, 75.0%) as a yellow solid. MS: m/z: Calc'd for C₁₅H₁₉N₃O₅S₂[M+H−56]⁺330. found 330.

Step 3: To a stirred mixture of tert-butyl N-(5-{[(4-methylbenzenesulfonyl)oxy]methyl}-1,3,4-thiadiazol-2-yl)carbamate (500 mg, 1.29 mmol, 1 equiv) and diethylamine (190 mg, 2.59 mmol, 2 equiv) in DMF (15 mL) was added K₂CO₃ (538 mg, 3.89 mmol, 3 equiv). The resulting mixture was stirred for overnight at 80° C. The mixture was extracted with EtOAc (2×100 mL). The combined organic layers were washed with water (2×20 mL), dried over anhydrous Na₂SO₄. After filtration, the filtrate was concentrated under reduced pressure. The residue was purified by reversed-phase flash chromatography to afford tert-butyl N-{5-[(diethylamino)methyl]-1,3,4-thiadiazol-2-yl}carbamate (400 mg, 95.8%) as a yellow oil. MS: m/z: Calc'd for C₁₂H₂₂N₄O₂S [M+H]⁺287. found 287.

Step 4: To a stirred mixture of tert-butyl N-{5-[(diethylamino)methyl]-1,3,4-thiadiazol-2-yl}carbamate (300 mg, 1.04 mmol, 1 equiv) in DCM (5 mL) was added TFA (1 mL) at 0° C. The resulting mixture was stirred for overnight at room temperature. The mixture was concentrated under reduced pressure. This resulted in 5-[(diethylamino)methyl]-1,3,4-thiadiazol-2-amine (200 mg, 92.2%) as a yellow oil. MS: m/z: Calc'd for C₇H₁₄N₄S [M+H]⁺187. found 187.

Step 5: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1 eq) and 5-[(diethylamino)methyl]-1,3,4-thiadiazol-2-amine (63 mg, 0.34 mmol, 2 eq) in DMF (6 mL) was added Cs₂CO₃ (166 mg, 0.51 mmol, 3 eq). The resulting mixture was stirred for 12 h at room temperature. The mixture was filtered and the filtrate was purified by reversed-phase flash chromatography to afford tert-butyl (2R,3S,4S)-4-[(tert-butoxycarbonyl) oxy]-3-[({5-[(diethylamino)methyl]-1,3,4-thiadiazol-2-yl}carbamoyl)oxy]-2-[(4-methoxy phenyl)methyl]pyrrolidine-1-carboxylate (103 mg, 95.4%) as a colorless oil. MS: m/z: Calc'd for: C₃₀H₄₅N₅O₈S [M+H]⁺636. Found, 636.

Step 6: To a stirred solution of tert-butyl (2R,3S,4S)-4-[(tert-butoxycarbonyl)oxy]-3-[({5-[(diethyl amino)methyl]-1,3,4-thiadiazol-2-yl}carbamoyl)oxy]-2-[(4-methoxyphenyl) methyl]pyrrolidine-1-carboxylate (100 mg, 0.16 mmol, 1 eq) in DCM (5 mL) was added TFA (1 mL). The resulting mixture was stirred for 2 h at room temperature. The mixture was concentrated and the crude product (80 mg) was purified by Prep-HPLC to afford (2R,3S,4S)-4-hydroxy-2-[(4-methoxyphenyl)methyl]pyrrolidin-3-yl N-{5-[(diethylamino)methyl]-1,3,4-thiadiazol-2-yl}carbamate; trifluoroacetic acid (17.8 mg, 20.49%) as a white solid. MS: m/z: Calc'd for C₂₀H₂₉N₅O₄S [M+H]⁺436. Found, 436. ¹H NMR (400 MHz, Methanol-d₄) δ 7.30-7.21 (m, 2H), 6.95-6.87 (m, 2H), 5.19-5.13 (m, 1H), 4.81 (s, 2H), 4.55-4.50 (m, 1H), 4.36-4.27 (m, 1H), 3.78 (s, 3H), 3.64 (dd, J=12.7, 4.2 Hz, 1H), 3.40-3.33 (m, 4H), 3.29 (d, J=12.7 Hz, 1H), 3.17 (dd, J=14.4, 7.2 Hz, 1H), 3.05 (dd, J=14.3, 8.8 Hz, 1H), 1.41 (t, J=7.3 Hz, 6H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 20% B in 10 min; Wave Length: 254 nm/220 nm; RT1(min): 8.68.

### Synthetic Procedure of V187553

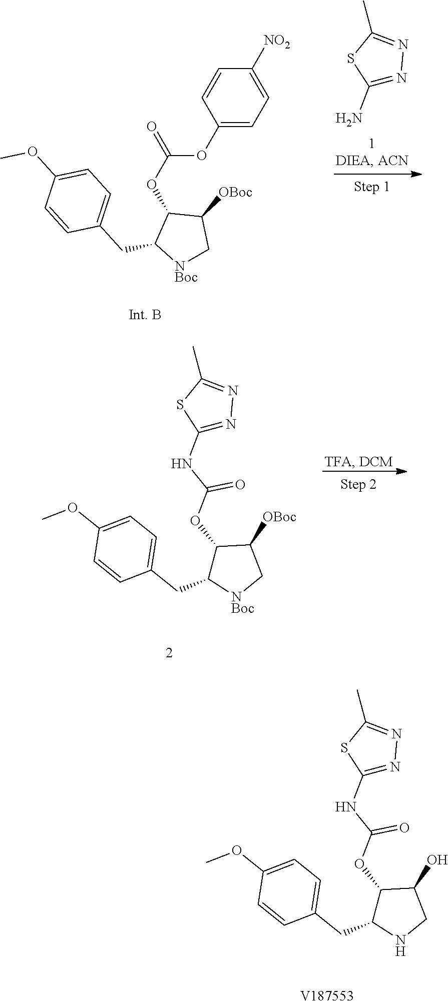

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq.) and 5-methyl-1,3,4-thiadiazol-2-amine (39 mg, 0.34 mmol, 2 eq.) in ACN (4 mL) was added DIEA (66 mg, 0.51 mmol, 3 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl)oxy]-2-[(4-methoxyphenyl)methyl]-3-{[(5-methyl-1,3,4-thiadiazol-2-yl)carbamoyl]oxy}pyrrolidine-1-carboxylate (90 mg, 93.8%) as a white solid. MS: m/z: Calc'd for C₂₆H₃₆N₄O₈S [M+H]⁺565. Found, 565.

Step 2: To a stirred solution of tert-butyl (2R,3S,4S)-4-[(tert-butoxycarbonyl) oxy]-2-[(4-methoxyphenyl) methyl]-3-{[(5-methyl-1,3,4-thiadiazol-2-yl) carbamoyl]oxy}pyrrolidine-1-carboxylate (90 mg, 0.16 mmol) in DCM (5 ml) was added TFA (5 mL) at 0° C. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-(5-methyl-1,3,4-thiadiazol-2-yl)carbamate; trifluoroacetic acid (14.8 mg, 19.3%) as a white solid. MS: m/z: Calc'd for C₁₆H₂₀N₄O₄S [M+H]⁺365. Found, 365. ¹H NMR (400 MHz, DMSO-d₆) δ 12.34 (s, 1H), 9.70 (s, 1H), 9.20 (s, 1H), 7.24 (d, J=8.4 Hz, 2H), 6.92-6.84 (m, 2H), 6.10 (s, 1H), 4.85 (d, J=3.8 Hz, 1H), 4.42-4.36 (m, 1H), 4.11 (s, 1H), 3.72 (s, 3H), 3.13 (d, J=12.4 Hz, 1H), 3.06 (dd, J=14.0, 7.5 Hz, 1H), 2.96 (dd, J=14.1, 7.9 Hz, 1H), 2.61 (s, 3H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 31% B in 10 min; Wave Length: 254 nm/220 nm; RT1(min): 8.68.

### Synthetic Procedure of V187554

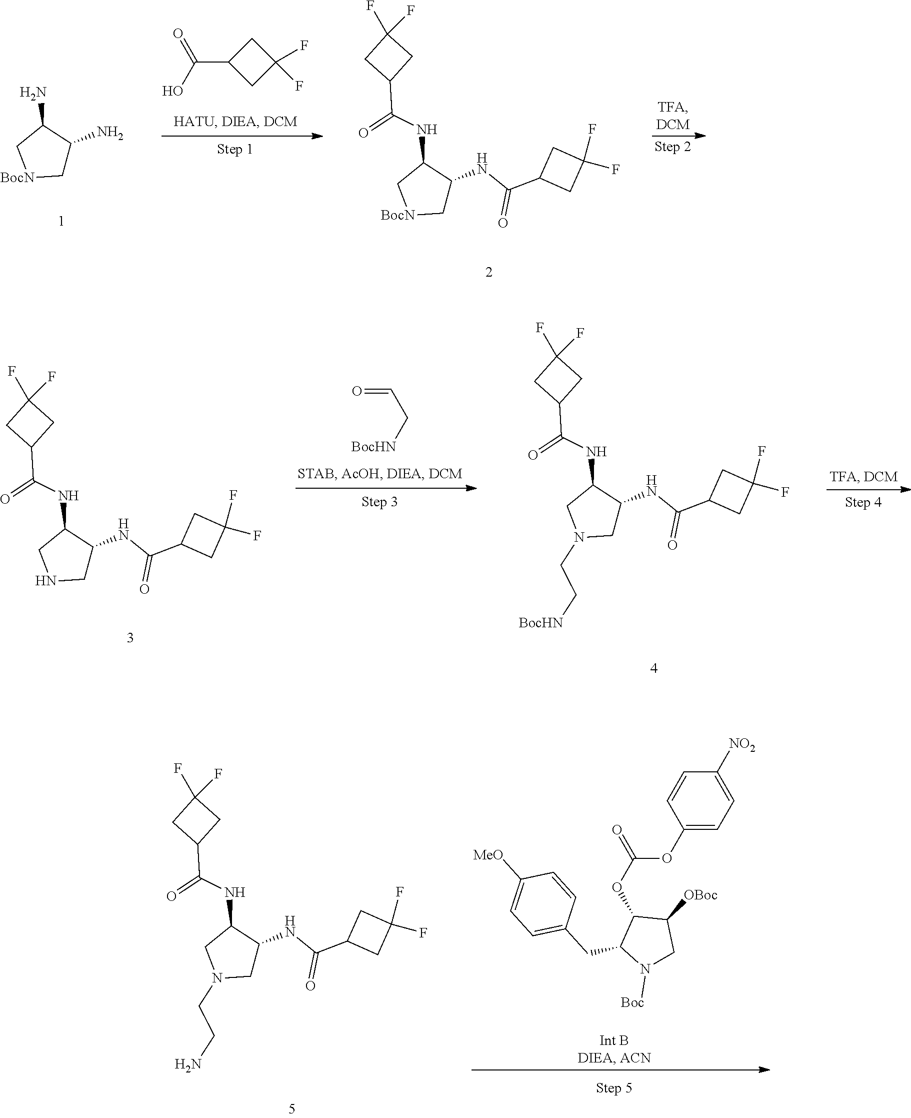

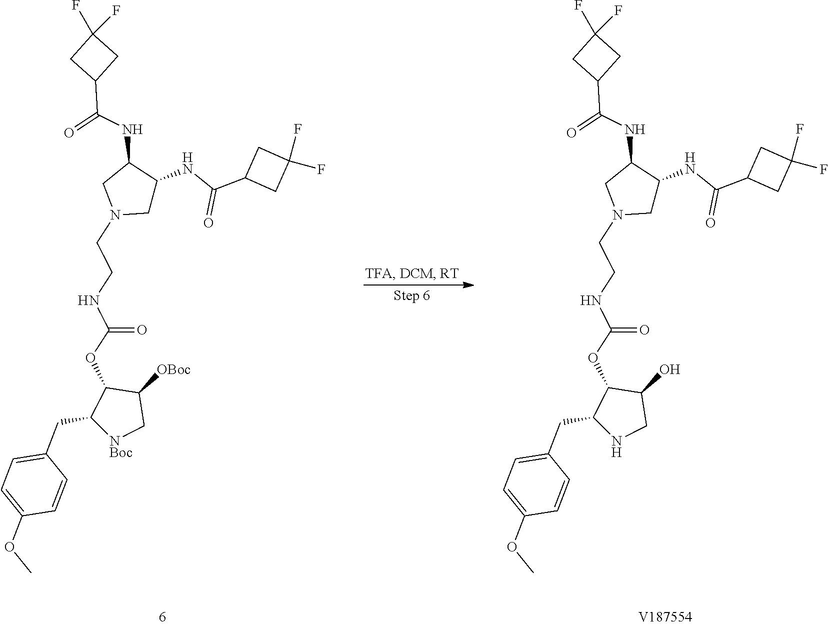

Step 1: A solution of 3,3-difluorocyclobutane-1-carboxylic acid (135 mg, 0.99 mmol, 2 equiv) and TCFH (209 mg, 0.74 mmol, 1.5 equiv) in ACN (5 mL) was treated with NMI (82 mg, 0.99 mmol, 2 equiv) for 30 min at 0° C. under nitrogen atmosphere followed by the addition of tert-butyl (3R,4R)-3,4-diaminopyrrolidine-1-carboxylate (100 mg, 0.49 mmol, 1 equiv). The resulting mixture was stirred for overnight at room temperature. The resulting mixture was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in tert-butyl (3R,4R)-3,4-bis(3,3-difluorocyclobutaneamido)pyrrolidine-1-carboxylate (150 mg, 69.0%) as a yellow solid. MS: m/z: Calc'd for C₁₉H₂₇F₄N₃O₄[M+H+22]⁺460. found 460.

Step 2: To a stirred solution of tert-butyl (3R,4R)-3,4-bis(3,3-difluorocyclobutane amido) pyrrolidine-1-carboxylate (120 mg, 0.27 mmol, 1 equiv) in DCM (5 mL) was added TFA (1 mL) dropwise at 0° C. The resulting mixture was stirred for 1 h at room temperature. Desired product could be detected by LCMS. The resulting mixture was concentrated under reduced pressure. This resulted in N-[(3R,4R)-4-(3,3-difluorocyclobutaneamido)pyrrolidin-3-yl]-3,3-difluorocyclobutane-1-carboxamide (80 mg, 86.4%) as a brown oil. MS: m/z: Calc'd for C₁₄H₁₉F₄N₃O₂[M+H]⁺338. found 338.

Step 3: A solution of N-[(3R,4R)-4-(3,3-difluorocyclobutaneamido)pyrrolidin-3-yl]-3,3-difluorocyclo butane-1-carboxamide (92 mg, 0.273 mmol, 1 equiv) and DIEA (177 mg, 1.36 mmol, 5 equiv) in DCM (5 mL) was treated with HOAc (115 mg, 1.91 mmol, 7 equiv) and tert-butyl N-(2-oxoethyl)carbamate (65 mg, 0.41 mmol, 1.5 equiv) for 1 h at 0° C. under nitrogen atmosphere followed by the addition of NaBH(OAc)₃ (116 mg, 0.55 mmol, 2 equiv). The resulting mixture was stirred for overnight at room temperature. Desired product could be detected by LCMS. The resulting mixture was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in tert-butyl N-{2-[(3R,4R)-3,4-bis(3,3-difluorocyclo butaneamido)pyrrolidin-1-yl]ethyl}carbamate (130 mg, 98.98%) as a black oil. MS: m/z: Calc'd for C₂₁H₃₂F₄N₄O₄[M+H]⁺481. found 481.

Step 4: To a stirred mixture of tert-butyl N-{2-[(3R,4R)-3,4-bis(3,3-difluorocyclobutaneamido) pyrrolidin-1-yl]ethyl}carbamate (146 mg, 0.30 mmol, 1 equiv) in DCM (5 mL) was added TFA (1 mL) dropwise at 0° C. under air atmosphere. The resulting mixture was stirred for 1 h at room temperature. Desired product could be detected by LCMS. The resulting mixture was concentrated under reduced pressure. to afford N-[(3R,4R)-1-(2-aminoethyl)-4-(3,3-difluorocyclobutaneamido)pyrrolidin-3-yl]-3,3-difluorocyclobutane-1-carboxamide (101 mg, 87.3%) as a black oil. MS: m/z: Calc'd for C₁₆H₂₄F₄N₄O₂[M+H]⁺381. found 381.

Step 5: To a stirred mixture of N-[(3R,4R)-1-(2-aminoethyl)-4-(3,3-difluorocyclobutane amido)pyrrolidin-3-yl]-3,3-difluorocyclobutane-1-carboxamide (129 mg, 0.34 mmol, 2 equiv) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.00 equiv) in ACN (5 mL) was added DIEA (66 mg, 0.51 mmol, 3 equiv). The mixture was stirred at rt for 2 hours. The mixture was concentrated under vacuum. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in tert-butyl (2R,3S,4S)-3-[({2-[(3R,4R)-3,4-bis(3,3-difluorocyclobutaneamido) pyrrolidin-1-yl]ethyl}carbamoyl)oxy]-4-[(tert-butoxycarbonyl)oxy]-2-[(4-methoxy phenyl)methyl]pyrrolidine-1-carboxylate (90 mg, 63.8%) as a yellow oil. MS: m/z: Calc'd for C₃₉H₅₅F₄N₅O₁₀ [M+H+22]⁺⁸⁵². found 852.

Step 6: To a stirred solution of tert-butyl (2R,3S,4S)-3-[({2-[(3R,4R)-3,4-bis(3,3-difluoro cyclobutaneamido)pyrrolidin-1-yl]ethyl}carbamoyl)oxy]-4-[(tert-butoxycarbonyl)oxy]-2-[(4-methoxyphenyl)methyl]pyrrolidine-1-carboxylate (60 mg, 0.07 mmol, 1 equiv) in DCM (5 mL) was added TFA (1 mL) at 0° C. The resulting mixture was stirred for 2 h at room temperature. The resulting mixture was concentrated under vacuum. The crude product (40 mg) was purified by Prep-HPLC to afford (2R,3S,4S)-4-hydroxy-2-[(4-methoxyphenyl)methyl]pyrrolidin-3-yl N-{2-[(3R,4R)-3,4-bis(3,3-difluorocyclobutaneamido)pyrrolidin-1-yl]ethyl}carbamate; trifluoroacetic acid (31.1 mg, 56.4%) as a brown solid. MS: m/z: Calc'd for C₂₉H₃₉F₄N₅O₆ [M+H+22]⁺630. found 630. ¹H NMR (400 MHz, Methanol-d₄) δ 7.28-7.20 (m, 2H), 6.97-6.89 (m, 2H), 5.00 (d, J=3.4 Hz, 1H), 4.52-4.33 (m, 3H), 4.17-4.05 (m, 1H), 3.92 (s, 2H), 3.79 (s, 3H), 3.66-3.30 (m, 7H), 3.21 (d, J=12.6 Hz, 1H), 3.13 (dd, J=14.3, 6.6 Hz, 1H), 3.04-2.86 (m, 3H), 2.87-2.62 (m, 8H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 6% B to 31% B in 10 min; Wave Length: 254 nm/220 nm; RT1(min): 8.68

### Synthetic Procedure of V187565

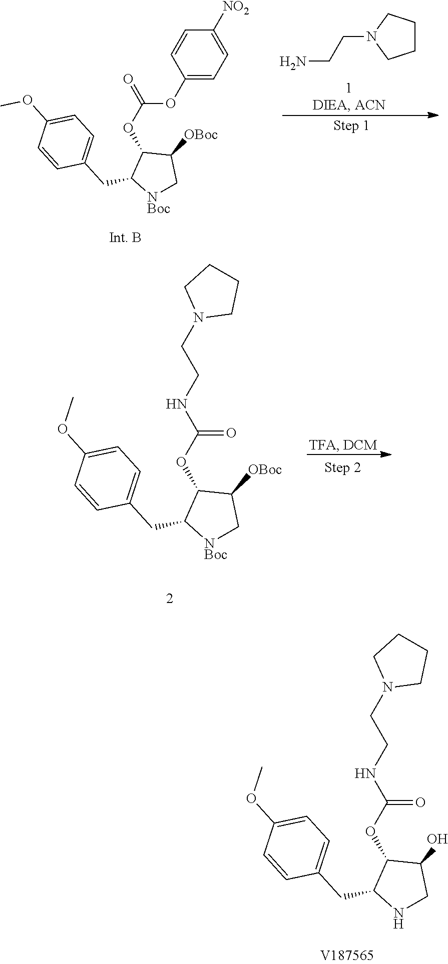

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (80 mg, 0.14 mmol, 1 eq.) and DIEA (52.7 mg, 0.41 mmol, 3 eq.) in ACN (5 mL) was added 1-pyrrolidineethanamine (15.5 mg, 0.14 mmol, 1 eq.) at room temperature under nitrogen atmosphere. The resulting mixture was stirred at room temperature for 12 h and the reaction mixture was concentrated in vacuo. The residue was purified by reversed-phase flash chromatography with the following conditions: column, C18 silica gel; mobile phase, ACN in Water (0.5% TFA), 10% to 80% gradient in 20 min; detector, UV 254 nm. This resulted in tert-butyl (2R,3S,4S)-4-[(tert-butoxycarbonyl)oxy]-2-[(4-methoxyphenyl)methyl]-3-({[2-(pyrrolidin-1-yl)ethyl]carbamoyl}oxy)pyrrolidine-1-carboxylate (65 mg, 84.8%) as a colorless oil. MS: m/z: Calc'd for C₂₉H₄₅N₃O₈ [M+H+22]⁺564. Found, 564.

Step 2: To a stirred solution of tert-butyl (2R,3S,4S)-4-[(tert-butoxycarbonyl)oxy]-2-[(4-methoxyphenyl)methyl]-3-({[2-(pyrrolidin-1-yl)ethyl]carbamoyl}oxy)pyrrolidine-1-carboxylate (60 mg, 0.11 mmol) in DCM (5 mL) was added TFA (1.0 mL) dropwise at 0° C. under nitrogen atmosphere. The resulting mixture was stirred at room temperature for 1 h. After completion of reaction monitored by LCMS, the mixture was concentrated. The crude product was purified by Prep-HPLC to afford (2R,3S,4S)-4-hydroxy-2-[(4-methoxyphenyl)methyl]pyrrolidin-3-yl N-[2-(pyrrolidin-1-yl)ethyl]carbamate; trifluoroacetic acid (16.7 mg, 32.8%) as a white solid. MS: m/z: Calc'd for C₁₉H₂₉N₃O₄ [M+H]⁺364. Found, 364. ¹H NMR (400 MHz, Methanol-d₄) δ 7.28-7.22 (m, 2H), 6.98-6.90 (m, 2H), 5.02 (d, J=3.5 Hz, 1H), 4.43 (d, J=4.1 Hz, 1H), 4.29-4.19 (m, 1H), 3.80 (s, 5H), 3.65-3.50 (m, 3H), 3.38 (t, J=6.1 Hz, 2H), 3.27-3.07 (m, 4H), 2.98 (dd, J=14.3, 9.0 Hz, 1H), 2.12 (d, J=40.0 Hz, 4H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 13% B in 10 min; Wave Length: 254 nm/220 nm; RT1(min): 8.68.

### Synthetic Procedure of V187566

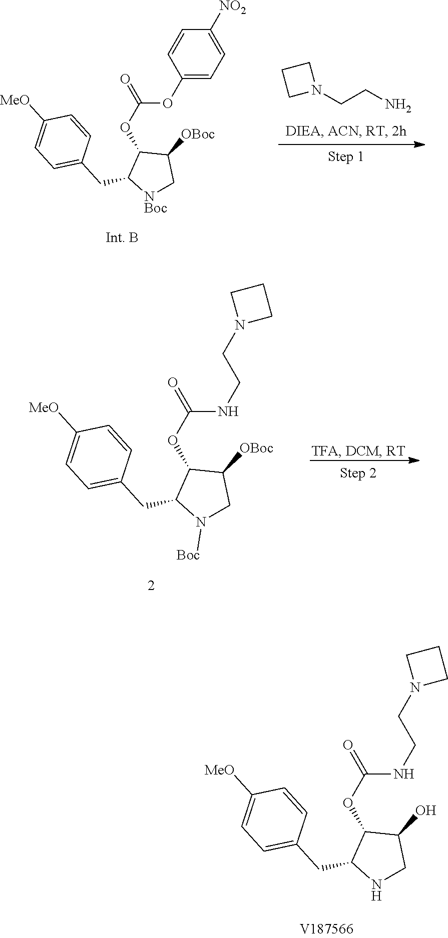

Step 1: To a stirred mixture of 2-(azetidin-1-yl)ethanamine (34 mg, 0.34 mmol, 2 equiv) in ACN (5 mL) was added tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.0 equiv) at 0° C. under N2 atmosphere. The resulting mixture was stirred at room temperature for overnight. After removing the solvent, the residue was purified by reversed-phase flash chromatography with the following conditions: column, C18 silica gel; mobile phase, MeCN in Water (0.05% TFA), 40% to 60% gradient in 10 min; detector, UV 254 nm. This resulted in tert-butyl (2R,3S,4S)-3-({[2-(azetidin-1-yl)ethyl]carbamoyl}oxy)-4-[(tert-butoxycarbonyl) oxy]-2-[(4-methoxyphenyl)methyl]pyrrolidine-1-carboxylate (116 mg, 124.2%) as a yellow oil. MS: m/z: Calc'd for C₂₈H₄₃N₃O₈ [M+H]⁺550. found 550.

Step 2: To a stirred solution of tert-butyl (2R,3S,4S)-3-({[2-(azetidin-1-yl)ethyl]carbamoyl}oxy)-4-[(tert-butoxycarbonyl)oxy]-2-[(4-methoxyphenyl)methyl]pyrrolidine-1-carboxylate (95 mg, 0.17 mmol, 1 equiv) in DCM (5 mL) was added TFA (1 mL) at 0° C. The resulting mixture was stirred for 1 h at room temperature and the mixture was concentrated under vacuum. The crude product (30 mg) was purified by Prep-HPLC to afford (2R,3S,4S)-4-hydroxy-2-[(4-methoxyphenyl)methyl]pyrrolidin-3-yl N-[2-(azetidin-1-yl)ethyl]carbamate; trifluoroacetic acid (20.9 mg, 25.9%) as a pink solid. MS: m/z: Calc'd for C₁₈H₂₇N₃O₄ [M+H]⁺350. found 350. ¹H NMR (400 MHz, Methanol-d₄) δ 7.28-7.20 (m, 2H), 6.97-6.89 (m, 2H), 5.00 (d, J=3.5 Hz, 1H), 4.46-4.40 (m, 1H), 4.35-4.08 (m, 5H), 3.79 (s, 3H), 3.57 (dd, J=12.7, 4.3 Hz, 1H), 3.51-3.33 (m, 4H), 3.21 (d, J=12.6 Hz, 1H), 3.11 (dd, J=14.4, 6.6 Hz, 1H), 2.98 (dd, J=14.3, 8.9 Hz, 1H), 2.52 (s, 2H). Prep-HPLC-conditions: Column: Atlantis T3 Prep OBD Column, 19*250 mm, 5 μm; Mobile Phase A: Water (0.05% TFA), Mobile Phase B: ACN; Flow rate: 25 mL/min mL/min; Gradient: 3% B to 25% B in 8 min; Wave Length: 254 nm/220 nm; RT1(min): 7.77

### Synthetic Procedure of V187568

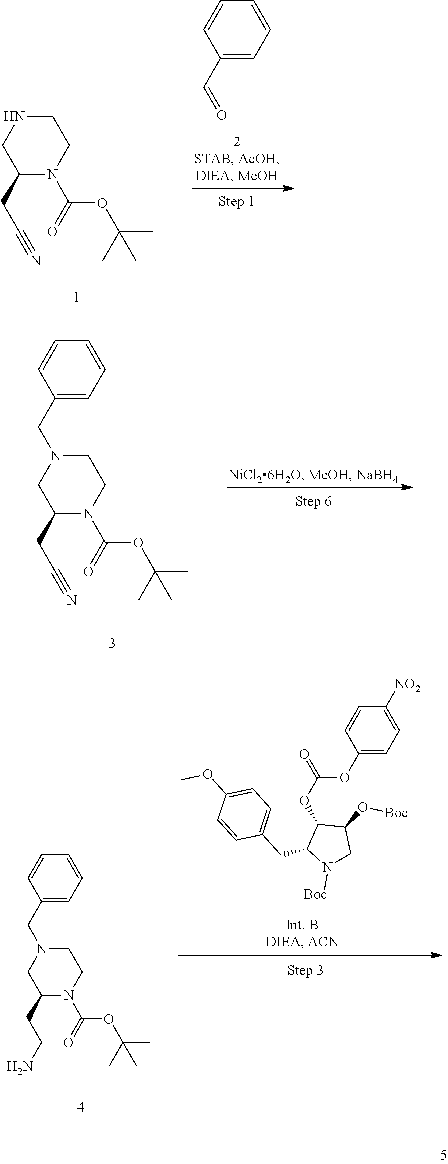 [SMILES: O=Cc1ccccc1 ; CC(C)(C)OC(=O)N1CCNC[C@@H]1CC#N ; CC(C)(C)OC(=O)N1CCN(Cc2ccccc2)C[C@@H]1CC#N ; CC(C)(C)OC(=O)N1CCN(Cc2ccccc2)C[C@@H]1CCN ; COc1ccc(C[C@@H]2[C@H](OC(=O)Oc3ccc([N+](=O)[O-])cc3)[C@@H](OC(=O)OC(C)(C)C)CN2C(=O)OC(C)(C)C)cc1]

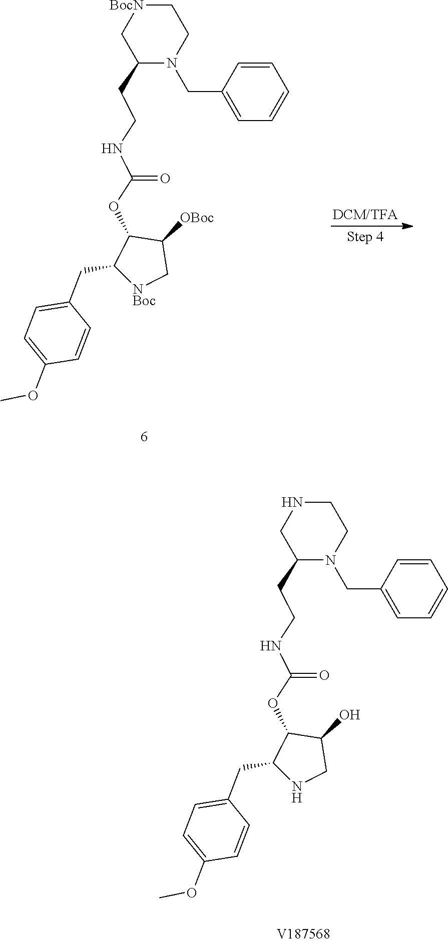

Step 1: To a stirred solution of 2,5-piperazinedione (tert-butyl (2S)-2-(cyanomethyl)piperazine-1-carboxylate (400 mg, 1.77 mmol, 1 eq.) and benzaldehyde (188 mg, 1.77 mmol, 1 eq.) in MeOH (6 mL) was added AcOH (213 mg, 3.55 mmol, 2 eq) at 0° C. The resulting reaction mixture was stirred at room temperature for 3h. NaBH(OAc)₃ (1129 mg, 5.32 mmol, 3 eq) was added to the mixture. The resulting mixture was stirred at room temperature for another 1 h. After completion of the reaction monitored by LCMS, the mixture was purified directly by reversed-phase column chromatography (10 mmol/L NH₄HCO₃ in water and acetonitrile) to afford tert-butyl (2S)-4-benzyl-2-(cyanomethyl)piperazine-1-carboxylate (270 mg, 48.2%) as a colorless oil. MS: m/z: Calc'd for C₁₈H₂₅N₃O₂ [M+H]⁺316. Found, 316.

Step 2: To a stirred solution of tert-butyl (2S)-4-benzyl-2-(cyanomethyl)piperazine-1-carboxylate (240 mg, 0.761 mmol, 1 eq) and NiCl₂·6H₂O (72.34 mg, 0.304 mmol, 0.4 eq) in MeOH (5 mL) was added a mixture of NaBH₄ (1295.31 mg, 34.245 mmol, 45 eq) in 5 mL MeOH dropwise at 0° C. The resulting mixture was stirred at room temperature for 2 h. After completion of the reaction monitored by LCMS, the resulting mixture was diluted with water (100 mL). The aqueous layer was extracted with EtOAc (3×200 mL). The combined organic layers were concentrated under reduced pressure. The residue was purified by reversed-phase column chromatography (10 mmol/L NH₄HCO₃ in water and acetonitrile) to afford tert-butyl (2S)-2-(2-aminoethyl)-4-benzylpiperazine-1-carboxylate (200 mg, 82.3%) as a light yellow oil. MS: m/z: Calc'd for C₁₈H₂₉N₃O₂ [M+H]⁺320. Found, 320.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq.) and tert-butyl (2S)-2-(2-aminoethyl)-4-benzylpiperazine-1-carboxylate (109 mg, 0.34 mmol, 2 eq.) in ACN (3 mL) was added DIEA (66 mg, 0.51 mmol, 3 eq.) at room temperature. The resulting mixture was stirred at room temperature for 3 h. After completion of the reaction monitored by LCMS, the resulting mixture was purified directly by reversed-phase column chromatography (10 mmol/L NH₄HCO₃ in water and acetonitrile) to afford tert-butyl (3S)-4-benzyl-3-{2-[({[(2R,3S,4S)-1-(tert-butoxycarbonyl)-4-[(tert-butoxycarbonyl)oxy]-2-[(4-methoxyphenyl)methyl]pyrrolidin-3-yl]oxy}carbonyl)amino]ethyl}piperazine-1-carboxylate (90 mg, 68.9%) as a white solid. MS: m/z: Calc'd for C₄₁H₆₀N₄O₁₀ [M+H]⁺769. Found, 769.

Step 4: To a stirred solution of tert-butyl (3S)-4-benzyl-3-{2-[({[(2R,3S,4S)-1-(tert-butoxycarbonyl)-4-[(tert-butoxycarbonyl) oxy]-2-[(4-methoxyphenyl) methyl]pyrrolidin-3-yl]oxy}carbonyl) amino]ethyl}piperazine-1-carboxylate (75 mg, 0.10 mmol) in DCM (5 mL) was added TFA (5 mL) at 0° C. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl N-{2-[(2S)-1-benzylpiperazin-2-yl]ethyl}carbamate (14.5 mg, 31.2%) as a white solid. MS: m/z: Calc'd for C₂₆H₃₆N₄O₄ [M+H]⁺469. Found, 469. ¹H NMR (400 MHz, Methanol-d₄) δ 7.36-7.26 (m, 4H), 7.30-7.24 (m, 1H), 7.13 (d, J=8.1 Hz, 2H), 6.85 (d, J=8.1 Hz, 2H), 4.62 (t, J=2.4 Hz, 1H), 4.15 (d, J=5.5 Hz, 1H), 3.76 (s, 3H), 3.53 (t, J=10.9 Hz, 3H), 3.30 (s, 1H), 3.21 (t, J=6.9 Hz, 2H), 2.94 (d, J=12.1 Hz, 1H), 2.79-2.54 (m, 7H), 2.15-2.04 (m, 1H), 1.84 (t, J=10.3 Hz, 1H), 1.57-1.53 (m, 2H). Prep-HPLC conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 16% B to 46% B in 7 min; Wave Length: 254 nm/220 nm; RT1(min): 6.6.

### Synthetic Procedure of V187575

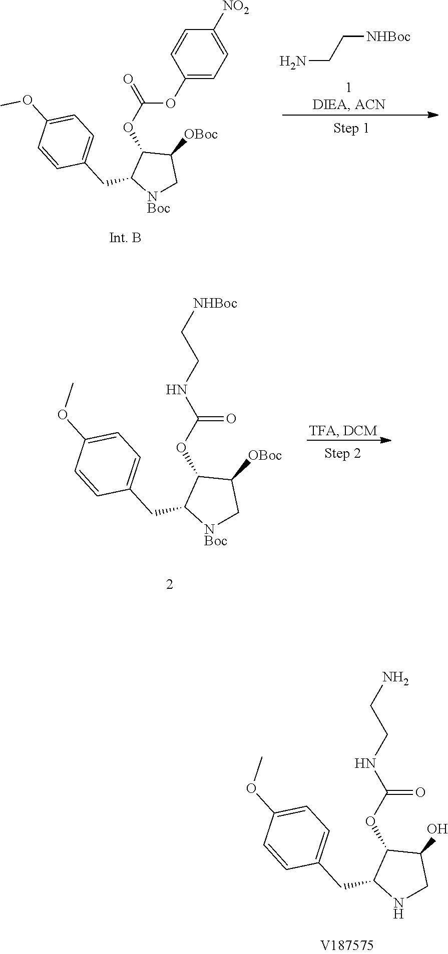

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int. B, 80 mg, 0.17 mmol, 1 eq.) and DIEA (52.7 mg, 0.41 mmol, 3 eq.) in ACN (5 mL) was added tert-butyl N-(2-aminoethyl)carbamate (21.8 mg, 0.14 mmol, 1 eq.) at room temperature under nitrogen atmosphere. The resulting mixture was stirred at room temperature for 12 h. After completion of reaction monitored by LCMS. The reaction was concentrated and the resulting residue was purified by reversed-phase flash chromatography with the following conditions: column, C18 silica gel; mobile phase, ACN in Water (0.5% TFA), 10% to 50% gradient in 20 min; detector, UV 254 nm. This resulted in tert-butyl (2R,3S,4S)-3-[({2-[(tert-butoxycarbonyl) amino]ethyl}carbamoyl)oxy]-4-[(tert-butoxycarbonyl)oxy]-2-[(4-methoxyphenyl)methyl]pyrrolidine-1-carboxylate (68 mg, 82.1%) as a colorless solid. MS: m/z: Calc'd for C₃₀H₄₇N₃O₁₀ [M+H+22]⁺632. Found, 632.

Step 2: To a stirred solution of tert-butyl (2R,3S,4S)-3-[({2-[(tert-butoxycarbonyl)amino]ethyl}carbamoyl)oxy]-4-[(tert-butoxycarbonyl)oxy]-2-[(4-methoxyphenyl) methyl]pyrrolidine-1-carboxylate (63 mg, 0.10 mmol) in DCM (5 mL) was added TFA (1 mL) dropwise at 0° C. under nitrogen atmosphere. The resulting mixture was stirred at room temperature for 2 h. After completion of reaction monitored by LCMS, the mixture was concentrated. The crude product (45 mg) was purified by Prep-HPLC to afford (2R,3S,4S)-4-hydroxy-2-[(4-methoxyphenyl) methyl]pyrrolidin-3-yl N-(2-aminoethyl)carbamate; trifluoroacetic acid (35.6 mg, 81.1%) as a off-white solid. MS: m/z: Calc'd for C₁₅H₂₃N₃O₄ [M+H]⁺410. Found, 410. ¹H NMR (400 MHz, Methanol-d₄) δ 7.25 (d, J=8.6 Hz, 2H), 6.93 (dd, J=8.4, 2.1 Hz, 2H), 4.99 (s, 1H), 4.43 (d, J=4.2 Hz, 1H), 4.30-4.10 (m, 1H), 3.79 (d, J=0.9 Hz, 3H), 3.59 (d, J=12.3 Hz, 1H), 3.55-3.38 (m, 2H), 3.21 (d, J=12.6 Hz, 1H), 3.15-3.07 (m, 3H), 2.99 (t, J=11.8 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 13% B in 10 min; Wave Length: 254 nm/220 nm; RT1(min): 8.68.

### Synthetic Procedure of V187576

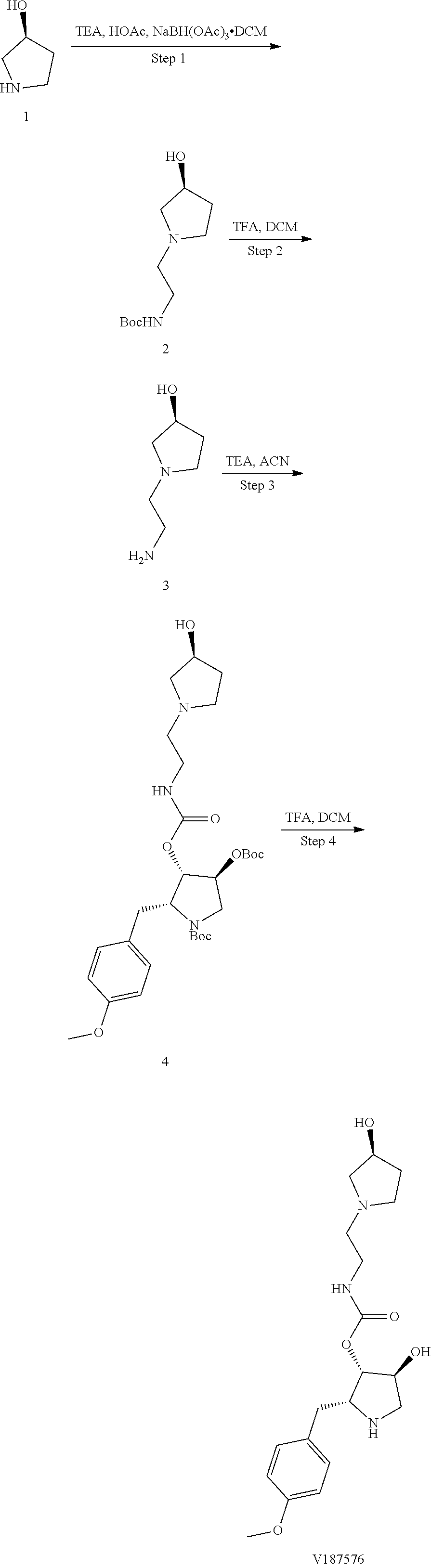

Step 1: To the solution of (3S)-pyrrolidin-3-ol (140 mg, 1.61 mmol, 1 eq.) and tert-butyl N-(2-oxoethyl)carbamate (281 mg, 1.77 mmol, 1.1 eq.) in DCM (4 mL) was added HOAc (10 mg, 0.16 mmol, 0.1 eq.). The mixture was stirred at r.t. for 0.5 h before NaBH(OAc)₃ (511 mg, 2.41 mmol, 1.5 eq.) was added. The mixture was stirred at r.t. for 5 h. Upon completion. The mixture was filtered and concentrated to give the crude product which was purified by reversed phase flash chromatography to afford tert-butyl N-{2-[(3S)-3-hydroxypyrrolidin-1-yl]ethyl}carbamate (30 mg, 8.1%) as a light yellow oil. MS: m/z: Calc'd for C₁₁H₂₂N₂O₃ [M+H]⁺231. found 231.

Step 2: To the solution of tert-butyl N-{2-[(3S)-3-hydroxypyrrolidin-1-yl]ethyl}carbamate (80 mg, 0.35 mmol) in DCM (3 mL) was added TFA (1 mL). The mixture was stirred at r.t. for 1 h. Upon completion. The mixture was concentrated to give the crude product, which was used directly in the next step without further purification. MS: m/z: Calc'd for C₆H₁₄N₂O [M+H]⁺131. found 131.

Step 3: To the solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (90 mg, 0.15 mmol) and (3S)-1-(2-aminoethyl)pyrrolidin-3-ol (40 mg, 0.31 mmol, 1 eq.) in ACN (1 mL) was added TEA (77 mg, 0.76 mmol, 2 eq.). The mixture was stirred at r.t. for 2 h. Upon completion. The mixture was concentrated to give the crude product which was used directly in the next step without further purification. MS: m/z: Calc'd for C₂₉H₄₅N₃O₉ [M+H]⁺580. found 580.

Step 4: To the solution of tert-butyl (2R,3S,4S)-4-[(tert-butoxycarbonyl)oxy]-3-[({2-[(3S)-3-hydroxypyrrolidin-1-yl]ethyl}carbamoyl)oxy]-2-[(4-methoxyphenyl)methyl]pyrrolidine-1-carboxylate (84 mg, 0.14 mmol) in DCM (5 mL) was added TFA (1 mL). The mixture was stirred at r.t. for 1 h. Upon completion. The mixture was concentrated to give the crude product which was purified by Prep HPLC to obtained (2R,3S,4S)-4-hydroxy-2-[(4-methoxyphenyl) methyl]pyrrolidin-3-yl N-{2-[(3S)-3-hydroxypyrrolidin-1-yl]ethyl}carbamate trifluoroacetic acid salt (18.1 mg, 24.4%) as a colorless semi-solid. MS: m/z: Calc'd for C₁₉H₂₉N₃O₅ [M+H]⁺380. found 380. ¹H NMR (400 MHz, Methanol-d₄) δ 7.29-7.20 (m, 2H), 6.98-6.89 (m, 2H), 5.01 (s, 1H), 4.59 (s, 1H), 4.44 (d, J=6.3 Hz, 1H), 4.24-4.13 (m, 1H), 3.79 (s, 3H), 3.65-3.49 (m, 3H), 3.50-3.34 (m, 3H), 3.26-3.17 (m, 2H), 3.16-2.88 (m, 3H), 2.40-2.36 (m, 1H), 2.12-2.08 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 14% B in 10 min; Wave Length: 254 nm/220 nm; RT1(min): 8.68.

### Synthetic Procedure of V187577

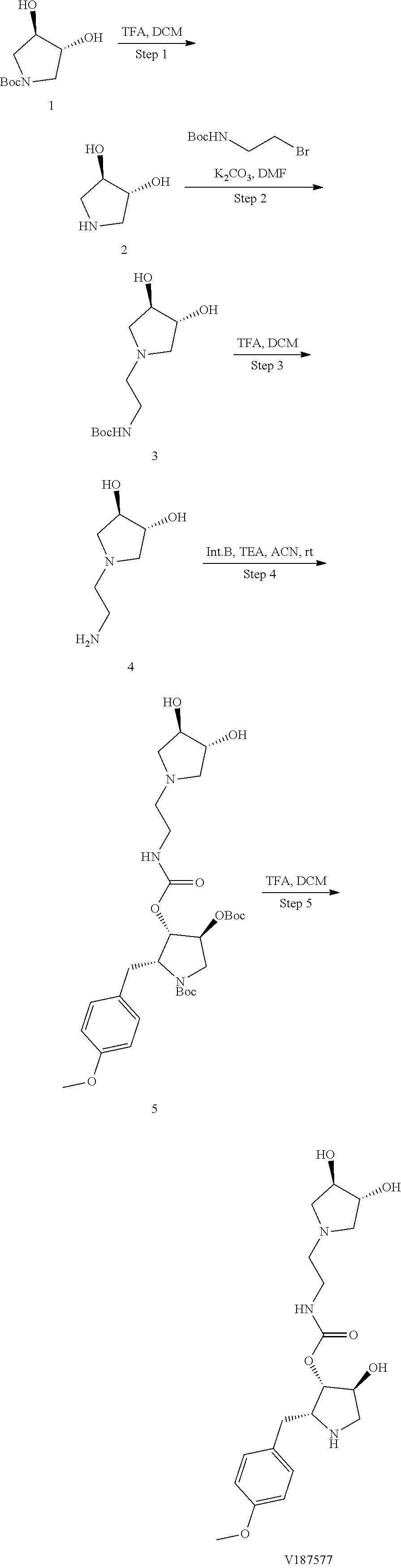

Step 1: To the solution of tert-butyl (3R,4R)-3,4-dihydroxypyrrolidine-1-carboxylate (300 mg, 1.47 mmol) in DCM (4 mL) was added TFA (2 mL). The mixture was stirred at r.t. for 1 h. Upon completion. The mixture was concentrated to give the crude product which was used directly in the next step without further purification. MS: m/z: Calc'd for C₄H₉NO₂ [M+H]⁺104. found 104.

Step 2: To the solution of (3R,4R)-pyrrolidine-3,4-diol (100 mg, 0.97 mmol) and tert-butyl N-(2-bromoethyl)carbamate (261 mg, 1.16 mmol, 1 eq.) in DMF (3 mL) was added K₂CO₃ (268 mg, 1.94 mmol, 1.6 eq.). The mixture was stirred at r.t. for overnight. Upon completion. The mixture was filtered and purified by reversed phase flash chromatography to afford tert-butyl N-{2-[(3R,4R)-3,4-dihydroxypyrrolidin-1-yl]ethyl}carbamate (47 mg, 19.7%) as a light yellow oil. MS: m/z: Calc'd for C₁₁H₂₂N₂O₄ [M+H]⁺247. found 247.

Step 3: To the solution of tert-butyl N-{2-[(3R,4R)-3,4-dihydroxypyrrolidin-1-yl]ethyl}carbamate (30 mg, 0.12 mmol) in DCM (3 mL) was added TFA (0.5 mL). The mixture was stirred at r.t. for 1 h. Upon completion. The mixture was concentrated to give the crude product which was used directly in the next step without further purification. MS: m/z: Calc'd for C₆H₁₄N₂O₂ [M+H]⁺147. found 147.

Step 4: To the solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int B, 75 mg, 0.13 mmol, 1 eq.) and (3R,4R)-1-(2-aminoethyl)pyrrolidine-3,4-diol (22 mg, 0.15 mmol, 1.1 eq.) in ACN (3 mL) was added TEA (26 mg, 0.25 mmol, 1.6 eq.). The mixture was stirred at r.t. for 2h. Upon completion. The mixture was concentrated to give the crude product which was purified by reverse phase flash. The tert-butyl (2R,3S,4S)-4-[(tert-butoxycarbonyl)oxy]-3-[({2-[(3R,4R)-3,4-dihydroxypyrrolidin-1-yl]ethyl}carbamoyl)oxy]-2-[(4-methoxyphenyl)methyl]pyrrolidine-1-carboxylate (50 mg, 65.9%) was obtained as a colorless oil. MS: m/z: Calc'd for C₂₉H₄₅N₃O₁₀ [M+H]⁺596. found 596.

Step 5: To the solution of tert-butyl (2R,3S,4S)-4-[(tert-butoxycarbonyl)oxy]-3-[({2-[(3R,4R)-3,4-dihydroxypyrrolidin-1-yl]ethyl}carbamoyl)oxy]-2-[(4-methoxyphenyl)methyl]pyrrolidine-1-carboxylate (50 mg, 0.08 mmol) in DCM (5 mL) was added TFA (1 mL). The mixture was stirred at r.t. for 1 h. Upon completion. The mixture was concentrated to give the crude product which was purified by Prep HPLC to obtained (2R,3S,4S)-4-hydroxy-2-[(4-methoxyphenyl) methyl]pyrrolidin-3-yl N-{2-[(3R,4R)-3,4-dihydroxypyrrolidin-1-yl]ethyl}carbamate (10.9 mg, 31.2%) as a grey solid. MS: m/z: Calc'd for C₁₉H₂₉N₃O₆ [M+H]⁺396. found 396. ¹H NMR (400 MHz, Methanol-d₄) δ 7.29-7.20 (m, 2H), 6.98-6.89 (m, 2H), 5.09 (s, 1H), 4.43 (d, J=4.1 Hz, 1H), 4.29 (d, J=3.7 Hz, 2H), 4.23-4.14 (m, 1H), 3.83-3.78 (m, 4H), 3.69-3.48 (m, 4H), 3.51-3.36 (m, 3H), 3.23 (dd, J=12.6, 3.7 Hz, 2H), 3.18-3.07 (m, 1H), 3.05-2.94 (m, 1H). Prep-HPLC-conditions: Column: Atlantis T3 Prep OBD Column, 19*250 mm, 5 μm; Mobile Phase A: Water (0.05% TFA), Mobile Phase B: ACN; Flow rate: 25 mL/min mL/min; Gradient: 3% B to 25% B in 8 min; Wave Length: 254 nm/220 nm; RT1(min): 7.62.

### Synthetic Procedure of V187578

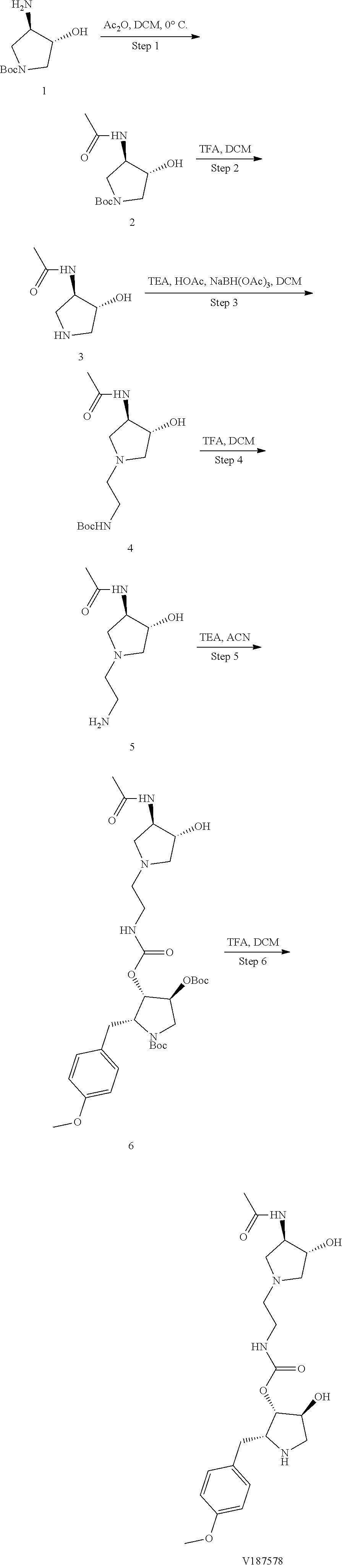

Step 1: To the solution of tert-butyl (3R,4R)-3-amino-4-hydroxypyrrolidine-1-carboxylate (250 mg, 1.24 mmol) in DCM (5 mL) was added acetic anhydride (139 mg, 1.36 mmol) at 0° C. The mixture was stirred at 0° C. for 2 hours. LCMS showed the reaction was complete. The mixture was concentrated to give the crude product which was used in the next step without further purification. MS: m/z: Calc'd for C₁₁H₂₀N₂O₄ [M−H]⁻243. found 243.

Step 2: To the solution of tert-butyl (3R,4R)-3-amino-4-hydroxypyrrolidine-1-carboxylate (300 mg, 1.22 mmol) in DCM (4 mL) was added TFA (2 mL) slowly. The mixture was stirred at rt for 1 hour. LCMS showed the reaction was complete. The mixture was concentrated to give the crude which was used directly in the next step. MS: m/z: Calc'd for C₆H₁₂N₂O₂ [M+H]⁺145. found 145.

Step 3: To the solution of N-[(3R,4R)-4-hydroxypyrrolidin-3-yl]acetamide (100 mg, 0.69 mmol) and tert-butyl N-(2-oxoethyl)carbamate (132 mg, 0.83 mmol) in DCM (5 mL) were added TEA (140 mg, 1.39 mmol) and HOAc (4.17 mg, 0.069 mmol). The mixture was stirred at rt for 0.5 hour before NaBH(OAc)₃ (294 mg, 1.39 mmol) was added. The mixture was stirred for another 0.5 hour. Upon completion, the reaction mixture was concentrated in vacuo. The resulting residue was dissolved with DMSO and purified by a reversed-phase column chromatography (0.05% TFA in water and acetonitrile) to afford N-{2-[(3R,4R)-3-acetamido-4-hydroxypyrrolidin-1-yl]ethyl}carbamate (90 mg, 45.2%) was obtained as a light yellow oil. MS: m/z: Calc'd for C₁₃H₂₅N₃O₄ [M+H]⁺288. found 288.

Step 4: To the solution of tert-butyl N-{2-[(3R,4R)-3-acetamido-4-hydroxypyrrolidin-1-yl]ethyl}carbamate (90 mg, 0.31 mmol) in DCM (3 mL) was added TFA (1 mL) slowly. The mixture was stirred at rt for 1 hour. Upon completion, the reaction mixture was concentrated in vacuo to afford the crude product which was used in the next step without further purification. MS: m/z: Calc'd for C₈H₁₇N₃O₂ [M+H]⁺188. found 188.

Step 5: To the solution of tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (80 mg, 0.14 mmol) and N-[(3R,4R)-1-(2-aminoethyl)-4-hydroxypyrrolidin-3-yl]acetamide (31 mg, 0.10 mmol) in ACN (1 mL) was added TEA (41 mg, 0.41 mmol). The mixture was stirred at rt for 2 hours. Upon completion, the reaction mixture was concentrated in vacuo. The resulting residue was dissolved with DMSO and purified by a reversed-phase column chromatography (0.05% TFA in water and acetonitrile) to afford tert-butyl (2R,3S,4S)-4-[(tert-butoxycarbonyl)oxy]-3-[({2-[(3R,4R)-3-acetamido-4-hydroxypyrrolidin-1-yl]ethyl}carbamoyl)oxy]-2-[(4-methoxyphenyl) methyl]pyrrolidine-1-carboxylate (84 mg, 97.1%) was obtained as a colorless oil. MS: m/z: Calc'd for C₃₁H₄₈N₄O₁₀ [M+H]⁺637. found 637.

Step 6: To the solution of tert-butyl (2R,3S,4S)-4-[(tert-butoxycarbonyl)oxy]-3-[({2-[(3R,4R)-3-acetamido-4-hydroxypyrrolidin-1-yl]ethyl}carbamoyl)oxy]-2-[(4-methoxyphenyl)methyl]pyrrolidine-1-carboxylate (84 mg, 0.13 mmol) in DCM (5 mL) was added TFA (1 mL) slowly. The mixture was stirred at rt for 1 hour. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-{2-[(3R,4R)-3-acetamido-4-hydroxypyrrolidin-1-yl]ethyl}carbamate (16.9 mg, 23.3%) was obtained as an off-white solid. MS: m/z: Calc'd for C₂₁H₃₂N₄O₆ [M+H]⁺437. found 437. ¹H NMR (400 MHz, Methanol-d₄) δ 7.29-7.21 (m, 2H), 6.98-6.90 (m, 2H), 5.01 (d, J=3.5 Hz, 1H), 4.43 (dd, J=15.3, 4.0 Hz, 2H), 4.23-4.14 (m, 3H), 3.80 (s, 3H), 3.67-3.52 (m, 4H), 3.54-3.36 (m, 3H), 3.22 (d, J=12.6 Hz, 2H), 3.13 (dd, J=14.3, 6.6 Hz, 1H), 2.98 (dd, J=14.3, 8.9 Hz, 1H), 1.98 (s, 3H). Prep-HPLC-conditions: Column: Column: XBridge Prep Shield RP18 OBD C18 Column, 30*150 mm, 5 μm; Mobile Phase A: Water (0.05% TFA), Mobile Phase B: ACN; Flow rate: 60 mL/min mL/min; Gradient: 1% B to 20% B in 10 min; Wave Length: 254 nm/220 nm; RT1(min): 8.5.

### Synthetic Procedure of V187579

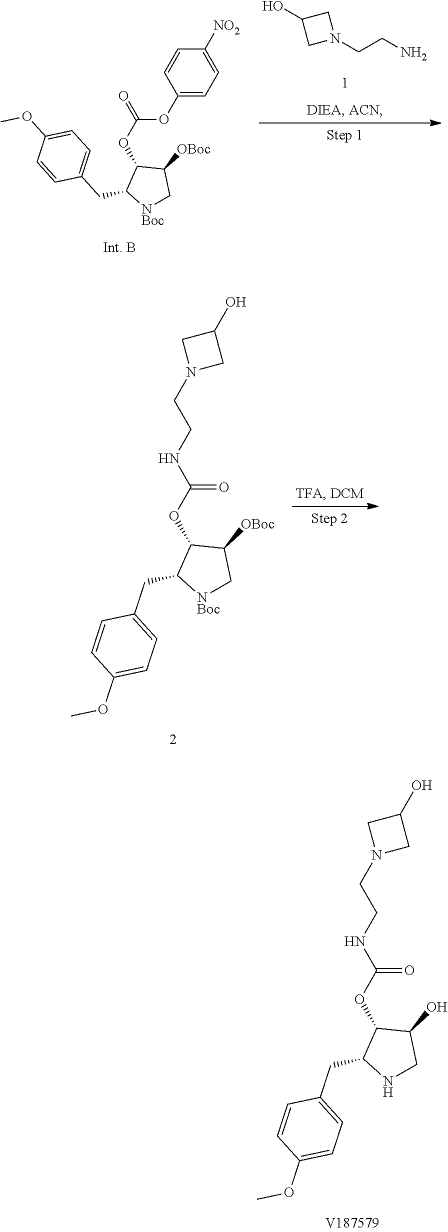

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int. B, 80 mg, 0.14 mmol, 1 eq.) and DIEA (52.7 mg, 0.41 mmol, 3 eq.) in ACN (5 mL) was added 1-(2-aminoethyl)azetidin-3-ol (15.8 mg, 0.14 mmol, 1 eq.) at room temperature under nitrogen atmosphere. The resulting mixture was stirred at room temperature for 12 h under nitrogen atmosphere. After completion of reaction monitored by LCMS. The solvent was removed and the residue was purified by reversed-phase flash chromatography with the following conditions: column, C18 silica gel; mobile phase, ACN in Water (0.5% TFA), 10% to 50% gradient in 20 min; detector, UV 254 nm. This resulted in tert-butyl (2R,3S,4S)-4-[(tert-butoxycarbonyl)oxy]-3-({[2-(3-hydroxyazetidin-1-yl)ethyl]carbamoyl}oxy)-2-[(4-methoxyphenyl)methyl]pyrrolidine-1-carboxylate TFA salt (100 mg, 97.1%) as a light yellow oil. MS: m/z: Calc'd for C₂₈H₄₃N₃O₉ [M+H]⁺566. Found, 566.

Step 2: To a stirred solution of tert-butyl (2R,3S,4S)-4-[(tert-butoxycarbonyl)oxy]-3-({[2-(3-hydroxyazetidin-1-yl)ethyl]carbamoyl}oxy)-2-[(4-methoxyphenyl)methyl]pyrrolidine-1-carboxylate (100 mg, 0.18 mmol) in 1,4-dioxane (3 mL) was added HCl (gas) in 1,4-dioxane (3 mL) dropwise at 0° C. under nitrogen atmosphere. The resulting mixture was stirred at room temperature for 2 h. After completion of reaction monitored by LCMS, the mixture was concentrated. The crude product (70 mg) was purified by Prep-HPLC to afford (2R,3S,4S)-4-hydroxy-2-[(4-methoxyphenyl)methyl]pyrrolidin-3-yl N-[2-(3-hydroxyazetidin-1-yl)ethyl]carbamate; trifluoroacetic acid (19.8 mg, 22.9%) as a light yellow solid. MS: m/z: Calc'd for C₁₈H₂₇N₃O₅ [M+H]⁺366. Found, 366. ¹H NMR (400 MHz, Methanol-d₄) δ 7.28-7.20 (m, 2H), 6.98-6.89 (m, 2H), 5.04-4.98 (m, 1H), 4.66 (t, J=6.1 Hz, 1H), 4.46-4.25 (m, 3H), 4.23-4.10 (m, 1H), 4.08-3.80 (s, 2H), 3.75 (s, 3H), 3.56 (dd, J=12.6, 4.3 Hz, 1H), 3.50-3.36 (m, 4H), 3.22 (d, J=12.6 Hz, 1H), 3.15-3.07 (m, 1H), 2.97 (dd, J=14.3, 9.0 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 15% B in 10 min; Wave Length: 254 nm/220 nm; RT1(min): 8.68.

### Synthetic Procedure of V187580

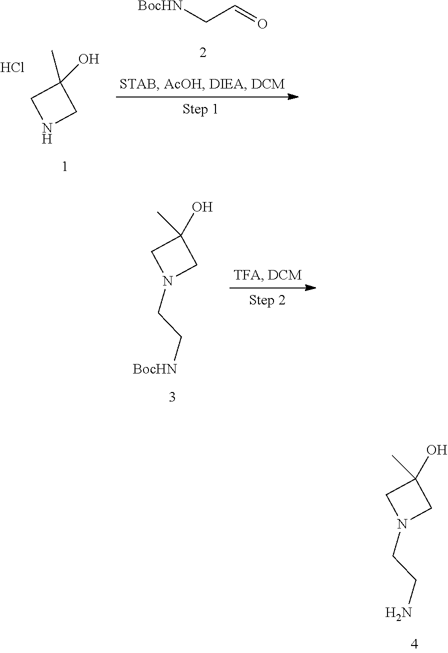

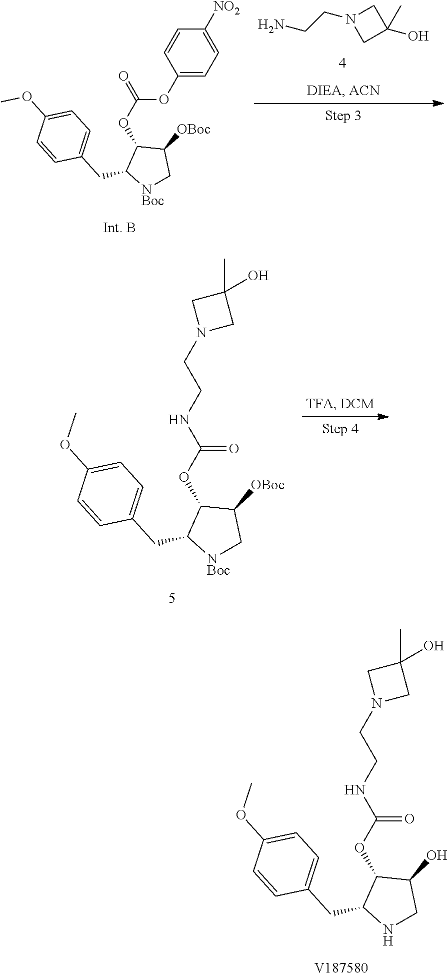

Step 1: A solution of 3-methylazetidin-3-ol hydrochloride (500 mg, 4.05 mmol, 1 eq.) in DCM (15 mL) was treated with TEA (1.2 g, 12.14 mmol, 3 eq) for 5 min at 0° C. under nitrogen atmosphere followed by the addition of AcOH (1.46 g, 24.28 mmol, 6 eq.) and tert-butyl N-(2-oxoethyl)carbamate (644.1 mg, 4.05 mmol, 1 eq.) at 0° C. The resulting mixture was stirred at room temperature for 1 h under nitrogen atmosphere. To the above mixture was added NaBH(OAc)₃ (1.72 g, 8.10 mmol, 2 eq.) in portions at 0° C. The resulting mixture was stirred for additional 2h at room temperature. Upon completion, the reaction mixture was concentrated in vacuo. The residue was purified by reversed-phase flash chromatography with the following conditions: column, C18 silica gel; mobile phase, ACN in Water (0.5% TFA), 10% to 50% gradient in 20 min; detector, UV 254 nm. This resulted in tert-butyl N-[2-(3-hydroxy-3-methylazetidin-1-yl)ethyl]carbamate (920 mg, 98.7%) as a light yellow oil. MS: m/z: Calc'd for C_{II}H₂₂N₂O₃ [M+H]⁺231. Found, 231.

Step 2: To a stirred solution of tert-butyl N-[2-(3-hydroxy-3-methylazetidin-1-yl)ethyl]carbamate (100 mg, 0.45 mmol) in DCM (5 mL) was added TFA (1 mL) dropwise at 0° C. under nitrogen atmosphere. The resulting mixture was stirred at room temperature under for 2 h. Upon completion, the reaction mixture was concentrated in vacuo. The crude product was used in the next step directly without further purification. MS: m/z: Calc'd for C₆H₁₄N₂O [M+H]⁺131. Found, 131.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int. B, 80 mg, 0.14 mmol, 1 eq) and 1-(2-aminoethyl)-3-methylazetidin-3-ol (17.7 mg, 0.14 mmol, 1 eq.) in ACN (5 mL) was added DIEA (52.7 mg, 0.41 mmol, 3 eq.) at room temperature under nitrogen atmosphere. The resulting mixture was stirred for 12 h. Upon completion, the reaction mixture was concentrated in vacuo. The residue was purified by reversed-phase flash chromatography with the following conditions: column, C18 silica gel; mobile phase, ACN in Water (0.5% TFA), 10% to 50% gradient in 20 min; detector, UV 254 nm. This resulted in tert-butyl (2R,3S,4S)-4-[(tert-butoxycarbonyl)oxy]-3-({[2-(3-hydroxy-3-methylazetidin-1-yl)ethyl]carbamoyl}oxy)-2-[(4-methoxyphenyl)methyl]pyrrolidine-1-carboxylate (28 mg, 35.5%) as a colorless oil. MS: m/z: Calc'd for C₂₉H₄₅N₃O₉ [M+H]⁺580. Found, 580.

Step 4: To a stirred solution of tert-butyl (2R,3S,4S)-4-[(tert-butoxycarbonyl)oxy]-3-({[2-(3-hydroxy-3-methylazetidin-1-yl)ethyl]carbamoyl}oxy)-2-[(4-methoxyphenyl)methyl]pyrrolidine-1-carboxylate (40 mg, 0.07 mmol) in DCM (5 mL) was added TFA (1 mL) dropwise at 0° C. under nitrogen atmosphere. The resulting mixture was stirred at room temperature for 2 h under nitrogen atmosphere. After completion of reaction monitored by LCMS. The crude product (30 mg) was purified by Prep-HPLC to afford (2R,3S,4S)-4-hydroxy-2-[(4-methoxyphenyl) methyl]pyrrolidin-3-yl N-[2-(3-hydroxy-3-methylazetidin-1-yl)ethyl]carbamate; trifluoroacetic acid (8.3 mg, 23.5%) as a colorless semi-solid. MS: m/z: Calc'd for C₁₉H₂₉N₃O₅ [M+H]⁺380. Found, 380. ¹H NMR (400 MHz, Methanol-d4) δ 7.24 (d, J=8.5 Hz, 2H), 6.94 (d, J=8.5 Hz, 2H), 5.02 (d, J=3.5 Hz, 1H), 4.43 (s, 1H), 4.20 (s, 1H), 4.05-4.03 (m, 3H), 3.80 (s, 3H), 3.57-3.55 (m, 1H), 3.53 (d, J=4.1 Hz, 2H), 3.41 (s, 3H), 3.23 (d, J=12.5 Hz, 1H), 3.11 (d, J=6.6 Hz, 1H), 2.96-2.94 (m, 1H), 1.53 (s, 3H). Prep-HPLC-conditions: Column: Welch Ultimate XB-C18 50*250, 10 μm; Mobile Phase A: Water (0.05% TFA), Mobile Phase B: ACN; Flow rate: 60 mL/min mL/min; Gradient: 2% B to 40% B in 20 min; Wave Length: 254 nm/220 nm; RT1(min): 15.

### Synthetic Procedure of V187581

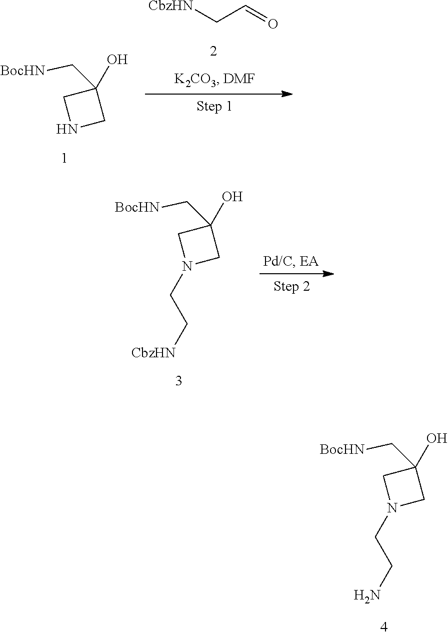 [SMILES: O=CCNC(=O)OCc1ccccc1 ; CC(C)(C)OC(=O)NCC1(O)CNC1 ; CC(C)(C)OC(=O)NCC1(O)CN(CCNC(=O)OCc2ccccc2)C1 ; CC(C)(C)OC(=O)NCC1(O)CN(CCN)C1]

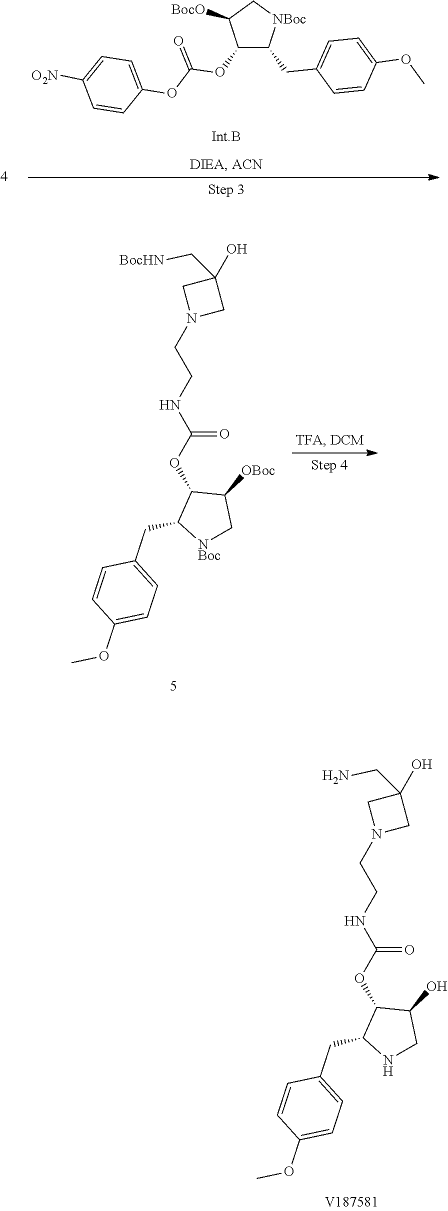

Step 1: To a stirred solution of tert-butyl N-[(3-hydroxyazetidin-3-yl)methyl]carbamate (300 mg, 1.48 mmol, 1.0 eq.) and benzyl N-(2-oxoethyl)carbamate (286.6 mg, 1.48 mmol, 1.0 eq.) in DMF (5 mL) was added K₂CO₃ (614.9 mg, 4.44 mmol, 3 equiv) in portions at room temperature. The resulting mixture was stirred over night at 25° C. under a nitrogen atmosphere. The resulting mixture was filtered, the filter cake was washed with DMF (1×1 mL). The residue was purified by reversed-phase flash chromatography with the following conditions: column, C18 silica gel; mobile phase, MeCN in Water (0.1% TFA), 10% to 50% gradient in 10 min; detector, UV 220 nm. This resulted in benzyl N-[2-(3-{[(tert-butoxycarbonyl)amino]methyl}-3-hydroxyazetidin-1-yl)ethyl]carbamate (300 mg, 53.3%) as a light yellow oil. MS: m/z: Calc'd for C₁₉H₂₉N₃O₅ [M+H]⁺380. found 380.

Step 2: Under a nitrogen atmosphere, Pd/C (100 mg) was added to a solution of benzyl N-[2-(3-{[(tert-butoxycarbonyl)amino]methyl}-3-hydroxyazetidin-1-yl)ethyl]carbamate (290 mg, 0.76 mmol, 1.0 eq.) in EA (5 mL), H2 was subsequently introduced into the reaction system, and the resulting mixture was stirred at r.t. for 2 h. Upon completion, the reaction mixture was filtered and concentrated in vacuo to afford crude product tert-butyl N-{[1-(2-aminoethyl)-3-hydroxyazetidin-3-yl]methyl}carbamate (180 mg, 96.0%) as a light yellow oil. MS: m/z: Calc'd for C₁₁H₂₃N₃O₃ [M+H]⁺246. found 246.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.14 mmol, 1.0 eq.) and tert-butyl N-{[1-(2-aminoethyl)-3-hydroxyazetidin-3-yl]methyl}carbamate (66.7 mg, 0.27 mmol, 2.0 eq.) in MeCN (3 mL) was added DIEA (52.7 mg, 0.41 mmol, 3 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-3-({[2-(3-{[(tert-butoxycarbonyl)amino]methyl}-3-hydroxyazetidin-1-yl)ethyl]carbamoyl}oxy)-4-[(tert-butoxycarbonyl)oxy]-2-[(4-methoxyphenyl) methyl]pyrrolidine-1-carboxylate (60 mg, 63.5%) as a light yellow oil. MS: m/z: Calc'd for C₂₇H₃₈N₄O₈ [M+H]⁺695. Found, 695.

Step 4: To a stirred mixture of tert-butyl (2R,3S,4S)-3-({[2-(3-{[(tert-butoxycarbonyl)amino]methyl}-3-hydroxyazetidin-1-yl)ethyl]carbamoyl}oxy)-4-[(tert-butoxycarbonyl)oxy]-2-[(4-methoxyphenyl)methyl]pyrrolidine-1-carboxylate (55 mg, 0.08 mmol, 1 eq.) in DCM (3 mL) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-{2-[3-(aminomethyl)-3-hydroxyazetidin-1-yl]ethyl}carbamate; trifluoroacetic acid (24.5 mg, 60.0%) as a white semi-solid. MS: m/z: Calc'd for C₁₉H₃₀N₄O₅ [M+H]⁺395. found 395. ¹H NMR (400 MHz, Methanol-d₄) δ 7.29-7.21 (m, 2H), 6.93 (d, J=8.2 Hz, 2H), 4.99 (s, 1H), 4.45 (d, J=4.9 Hz, 3H), 4.28-4.09 (m, 3H), 3.62-3.53 (m, 1H), 3.47 (dd, J=9.5, 6.0 Hz, 3H), 3.39 (s, 2H), 3.23-3.07 (m, 2H), 3.01-2.95 (m, 1H). Prep-HPLC-conditions: Column: Atlantis T3 Prep OBD Column, 19*250 mm, 5 μm; Mobile Phase A: Water (0.05% TFA), Mobile Phase B: ACN; Flow rate: 25 mL/min mL/min; Gradient: 3% B to 25% B in 8 min; Wave Length: 254 nm/220 nm; RT1(min): 7

### Synthetic Procedure of V187582

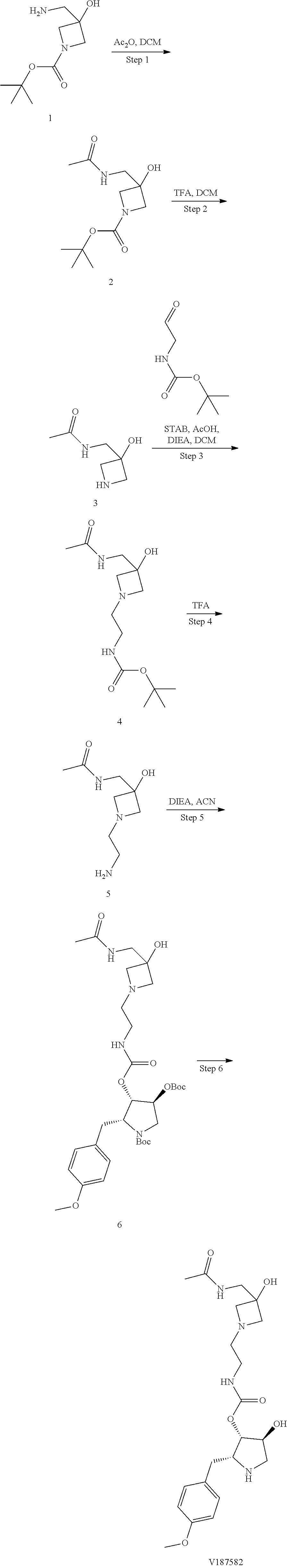

Step 1: To a stirred solution of tert-butyl 3-(aminomethyl)-3-hydroxyazetidine-1-carboxylate (300 mg, 1.483 mmol, 1 eq.) and Ac₂O (151.4 mg, 1.48 mmol, 1 eq.) in DCM (8 mL) was added DIEA (575.1 mg, 4.45 mmol, 3 eq.) and the resulting mixture was stirred at room temperature for 2 h. Upon completion, the reaction mixture was concentrated in vacuo. The resulting residue was dissolved with DMSO and purified by column chromatography to obtain tert-butyl 3-(acetamidomethyl)-3-hydroxyazetidine-1-carboxylate (200 mg, 55.2%) as a yellow oil. MS: m/z: Calc'd for C₁₁H₂₀N₂O₄ [M+H]⁺245. found 245.

Step 2: To a stirred solution of tert-butyl 3-(acetamidomethyl)-3-hydroxyazetidine-1-carboxylate (195 mg, 0.80 mmol, 1 eq.) in DCM (8 mL) was added TFA (1.6 mL) and the resulting mixture was stirred at room temperature for 2 h. LCMS showed the reaction was completed. The reaction mixture was concentrated in vacuo to obtain N-[(3-hydroxyazetidin-3-yl)methyl]acetamide (115 mg, 99.9%) as a yellow oil. MS: m/z: Calc'd for C₆H₁₂N₂O₂ [M+H]⁺145. found 145.

Step 3: To a stirred solution of N-[(3-hydroxyazetidin-3-yl)methyl]acetamide (145 mg, 1 mmol, 1 eq.) and tert-butyl N-(2-oxoethyl)carbamate (320.2 mg, 2 mmol, 2 eq.) in DCM (8 mL) was added AcOH (241.6 mg, 4.02 mmol, 4 eq.) at room temperature for 1 h. Then NaBH(OAc)₃ (426.31 mg, 2.02 mmol, 2 eq.) was added the mixture slowly at ice bath. The mixture was stirred at room temperature for 2 h. LCMS showed the reaction was completed. H₂O was added the mixture and extracted with DCM, the organic layer was concentrated. The crude was purified by column chromatography to obtain tert-butyl N-{2-[3-(acetamidomethyl)-3-hydroxyazetidin-1-yl]ethyl}carbamate (90 mg, 31.14%) as a yellow oil. MS: m/z: Calc'd for C₁₃H₂₅N₃O₄ [M+H]⁺288. found 288.

Step 4: To a stirred solution of tert-butyl N-{2-[3-(acetamidomethyl)-3-hydroxyazetidin-1-yl]ethyl}carbamate (90 mg, 0.32 mmol, 1 eq.) in DCM (5 mL) was added TFA (1 mL), and the resulting mixture was stirred at room temperature for 2 h. LCMS showed the reaction was completed. The reaction mixture was concentrated in vacuo to obtain N-{[1-(2-aminoethyl)-3-hydroxyazetidin-3-yl]methyl}acetamide (58 mg, 98.90%) as a brown oil. MS: m/z: Calc'd for C₈H₁₇N₃O₂ [M+H]⁺188. found 188.

Step 5: To a stirred solution of N-{[1-(2-aminoethyl)-3-hydroxyazetidin-3-yl]methyl}acetamide (60 mg, 0.32 mmol, 1 eq.) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int B, 188.6 mg, 0.32 mmol, 1 eq.) in ACN (7 mL) was added DIEA (124.2 mg, 0.96 mmol, 3 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl)oxy]-3-[({2-[3-(acetamidomethyl)-3-hydroxyazetidin-1-yl]ethyl}carbamoyl)oxy]-2-[(4-methoxyphenyl)methyl]pyrrolidine-1-carboxylate (60 mg, 29.41%) as a white solid. MS: m/z: Calc'd for C₃₁H₄₈N₄O₁₀ [M+H]⁺637. found 637.

Step 6: To a stirred solution of tert-butyl (2R,3S,4S)-4-[(tert-butoxycarbonyl)oxy]-3-[({2-[3-(acetamidomethyl)-3-hydroxyazetidin-1-yl]ethyl}carbamoyl)oxy]-2-[(4-methoxyphenyl) methyl]pyrrolidine-1-carboxylate (60 mg, 0.09 mmol, 1 eq.) in 1,4-dioxane (4 mL) was added HCl(gas) in 1,4-dioxane (4 M, 4 mL), and the resulting mixture was stirred at room temperature for 2 h. After completion of the reaction monitored by LCMS, the mixture was concentrated and purified by Prep-HPLC to obtain (2R,3S,4S)-4-hydroxy-2-[(4-methoxyphenyl)methyl]pyrrolidin-3-yl N-{2-[3-(acetamidomethyl)-3-hydroxyazetidin-1-yl]ethyl}carbamate hydrochloride (18.8 mg, 41.80%) as a light yellow solid. MS: m/z: Calc'd for C₂₁H₃₂N₄O₆ [M+H]⁺437. found 437. ¹H NMR (400 MHz, Methanol-d₄) δ 7.29 (dd, J=8.7, 2.8 Hz, 2H), 6.98-6.90 (m, 2H), 4.99-4.90 (m, 1H), 4.49 (dd, J=9.3, 5.4 Hz, 2H), 4.18 (s, 3H), 4.00 (dd, J=11.5, 8.4 Hz, 1H), 3.80 (s, 3H), 3.71-3.63 (m, 1H), 3.58 (d, J=2.2 Hz, 1H), 3.56-3.37 (m, 5H), 3.24-3.10 (m, 2H), 3.03 (dd, J=14.2, 8.9 Hz, 1H), 2.05 (d, J=1.4 Hz, 3H). Prep-HPLC-conditions: Column: Welch Ultimate XB-C18 50*250, 10 μm; Mobile Phase A: Water (0.05% HCL), Mobile Phase B: ACN; Flow rate: 60 mL/min mL/min; Gradient: 1% B to 30% B in 20 min; Wave Length: 254 nm/220 nm; RT1(min): 13

### Synthetic Procedure of V187583

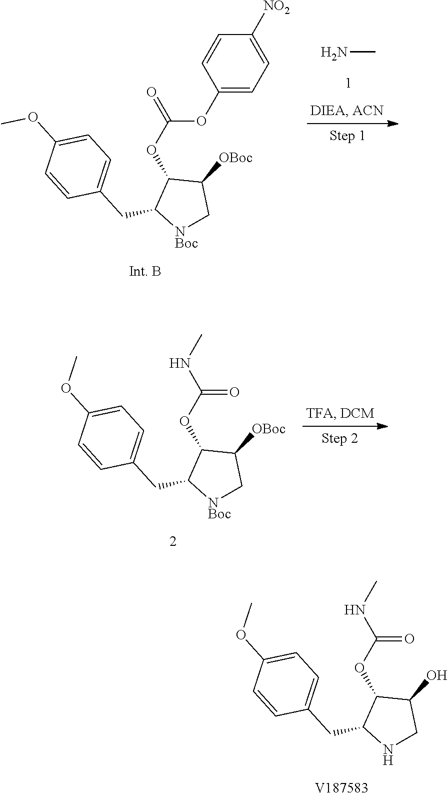

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (80 mg, 0.14 mmol, 1 eq.) and DIEA (35.1 mg, 0.27 mmol, 2 eq.) in ACN (5 mL) was added methylamine (4.2 mg, 0.14 mmol, 1 eq.) at room temperature under nitrogen atmosphere. The resulting mixture was stirred at room temperature for 12 h under nitrogen atmosphere. Upon completion, the reaction mixture was concentrated in vacuo. The residue was purified by reversed-phase flash chromatography with the following conditions: column, C18 silica gel; mobile phase, ACN in Water (0.5% TFA), 10% to 40% gradient in 14 min; detector, UV 254 nm. This resulted in tert-butyl (2R,3S,4S)-4-[(tert-butoxycarbonyl)oxy]-2-[(4-methoxyphenyl)methyl]-3-[(methyl carbamoyl)oxy]pyrrolidine-1-carboxylate (70 mg, 107.2%) as a colorless solid. MS: m/z: Calc'd for C₂₄H₃₆N₂O₈ [M+H−56−56]⁺369. Found, 369.

Step 2: To a stirred solution of tert-butyl (2R,3S,4S)-4-[(tert-butoxycarbonyl)oxy]-2-[(4-methoxyphenyl)methyl]-3-[(methylcarbamoyl)oxy]pyrrolidine-1-carboxylate (65 mg, 0.16 mmol) in DCM (5 mL) was added TFA (1 mL) dropwise at 0° C. under nitrogen atmosphere. The resulting mixture was stirred at room temperature for 2 h. After completion of reaction monitored by LCMS, the mixture was concentrated. The crude product (45 mg) was purified by Prep-HPLC to afford (2R,3S,4S)-4-hydroxy-2-[(4-methoxyphenyl)methyl]pyrrolidin-3-yl N-methylcarbamate; trifluoroacetic acid (21.0 mg, 37.8%) as a white solid. MS: m/z: Calc'd for C₁₄H₂₀N₂O₄ [M+H]⁺281; Found, 281. ¹H NMR (400 MHz, Methanol-d₄) δ 7.26-7.19 (m, 2H), 6.97-6.89 (m, 2H), 4.95 (d, J=3.5 Hz, 1H), 4.38 (d, J=4.3 Hz, 1H), 4.28-4.14 (m, 1H), 3.79 (s, 3H), 3.55 (dd, J=12.6, 4.4 Hz, 1H), 3.20 (d, J=12.6 Hz, 1H), 3.11-3.09 (m, 1H), 2.94-2.92 (m, 1H), 2.78 (s, 3H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 22% B in 10 min; Wave Length: 254 nm/220 nm; RT1(min): 8.68.

### Synthetic Procedure of V187584

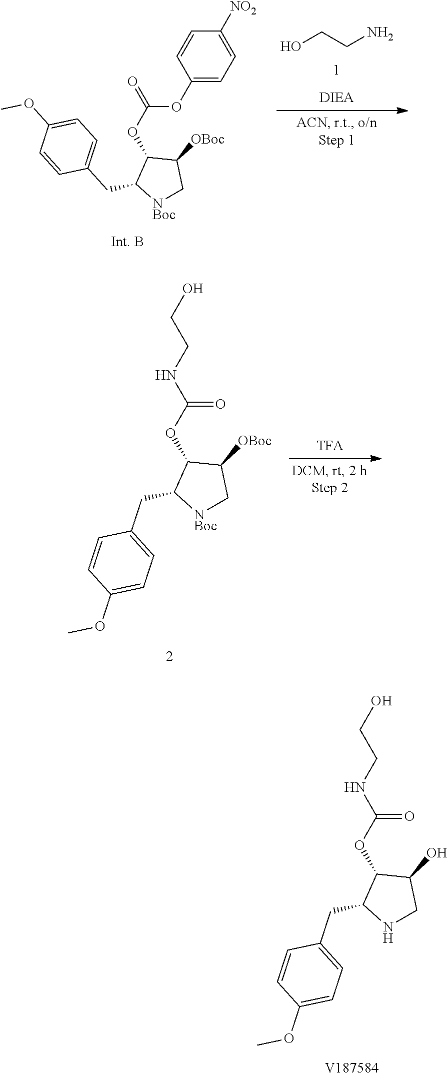

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.01 mmol, 1 eq.) in ACN (3 mL) were added DIEA (52.7 mg, 0.40 mmol, 3 eq.) and ethanolamine (8.3 mg, 0.13 mmol, 1 eq.) at 0° C. The reaction was stirred at r.t. for overnight. After completion of the reaction monitored by LCMS, The reaction mixture was quenched with water and extracted with EA. The extracts were washed with brine, dried over anhydrous sodium sulfate, filtered and concentrated. The residue was purified by Prep-HPLC (10 mmol/L NH₄HCO₃ in water and acetonitrile) to afford tert-butyl (2R,3S,4S)-4-[(tert-butoxycarbonyl) oxy]-3-{[(2-hydroxyethyl)carbamoyl]oxy}-2-[(4-methoxyphenyl) methyl]pyrrolidine-1-carboxylate (70 mg, 100.8%) as a white solid. MS: m/z: Calc'd for C₂₅H₃₈N₂O₉ [M−H]⁻509. Found, 509.

Step 2: To a stirred solution of tert-butyl (2R,3S,4S)-4-[(tert-butoxycarbonyl)oxy]-3-{[(2-hydroxyethyl)carbamoyl]oxy}-2-[(4-methoxyphenyl)methyl]pyrrolidine-1-carboxylate (60 mg, 0.11 mmol, 1 eq.) in DCM (3 mL) were added TFA (1 mL) dropwise at 0° C. The reaction was stirred at r.t. for 2 h. The resulting mixture was concentrated under reduced pressure. The crude product (60 mg) was purified by Prep-HPLC to afford (2R,3S,4S)-4-hydroxy-2-[(4-methoxy phenyl)methyl]pyrrolidin-3-yl N-(2-hydroxyethyl)carbamate (18.2 mg, 49.2%) as a yellow oil. MS: m/z: Calc'd for C₁₅H₂₂N₂O₅ [M+H]⁺311. Found, 311. ¹H NMR (400 MHz, Methanol-d4) δ 7.28-7.20 (m, 2H), 6.97-6.89 (m, 2H), 4.95 (d, J=3.4 Hz, 1H), 4.39 (d, J=4.4 Hz, 1H), 4.15-4.11 (m, 1H), 3.80 (s, 3H), 3.68-3.54 (m, 3H), 3.29 (t, J=5.7 Hz, 2H), 3.21 (d, J=12.6 Hz, 1H), 3.12-3.07 (m, 1H), 2.98-2.93 (m, 1H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 19% B in 10 min; Wave Length: 254 nm/220 nm; RT1(min): 8.68

### Synthetic Procedure of V187585

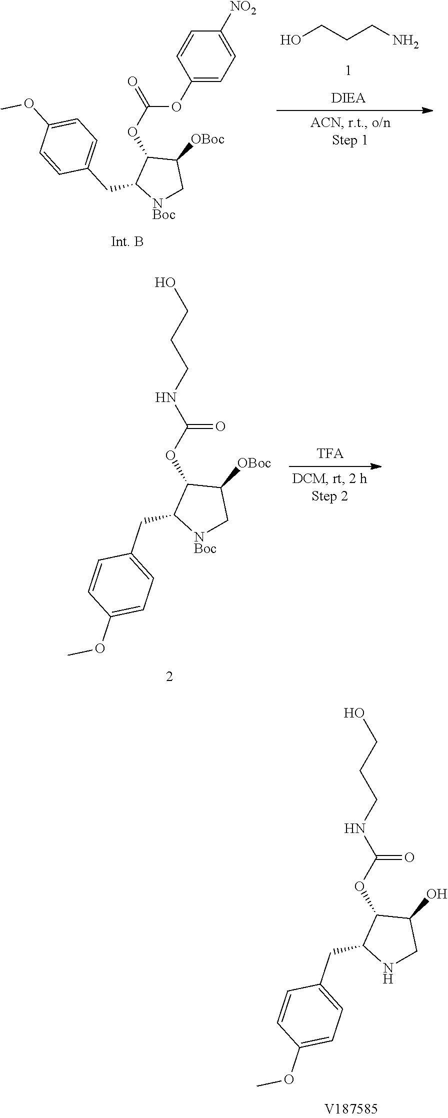

Step 1: To a stirred solution of tert-butyl (2R,3S,4S)-4-[(tert-butoxycarbonyl)oxy]-2-[(4-hydroxyphenyl)methyl]-3-[(4-nitrophenoxycarbonyl)oxy]pyrrolidine-1-carboxylate (Int.B, 80 mg, 0.01 mmol, 1 eq.) in ACN (3 mL) were added DIEA (53.9 mg, 0.41 mmol, 3 eq.) and propanolamine (15.6 mg, 0.20 mmol, 1.5 eq.) at 0° C. The reaction was stirred at r.t. for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl)oxy]-3-{[(3-hydroxypropyl) carbamoyl]oxy}-2-[(4-methoxyphenyl)methyl]pyrrolidine-1-carboxylate (70 mg, 95.8%) as a white solid. MS: m/z: Calc'd for C₂₆H₄₀N₂O₉[M−H]⁻523. Found, 523.

Step 2: To a stirred solution of tert-butyl (2R,3S,4S)-4-[(tert-butoxycarbonyl)oxy]-3-{1[(3-hydroxypropyl)carbamoyl]oxy}-2-[(4-methoxyphenyl)methyl]pyrrolidine-1-carboxylate (60 mg, 0.11 mmol, 1 eq.) in DCM (3 mL) were added TFA (1 mL) at 0° C. The reaction was stirred at r.t. for 2h. The resulting mixture was concentrated under reduced pressure. The crude product (60 mg) was purified by Prep-HPLC to afford (2R,3S,4S)-4-hydroxy-2-[(4-methoxyphenyl) methyl]pyrrolidin-3-yl N-(3-hydroxypropyl)carbamate (13.4 mg, 35.8%) as a yellow solid. MS: m/z: Calc'd for C₁₆H₂₄N₂O₅[M+H]⁺325. Found, 325. ¹H NMR (400 MHz, Methanol-d₄) δ 7.27-7.19 (m, 2H), 6.97-6.88 (m, 2H), 4.93 (d, J=3.1 Hz, 1H), 4.38 (d, J=4.2 Hz, 1H), 4.15-4.12 (m, 1H), 3.79 (s, 3H), 3.68-3.53 (m, 3H), 3.36-3.17 (m, 3H), 3.11-3.07 (m, 1H), 2.97-2.95 (m, 1H), 1.77-1.73 (m, 2H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 21% B in 10 min; Wave Length: 254 nm/220 nm; RT1(min): 8.68

### Synthetic Procedure of V187586

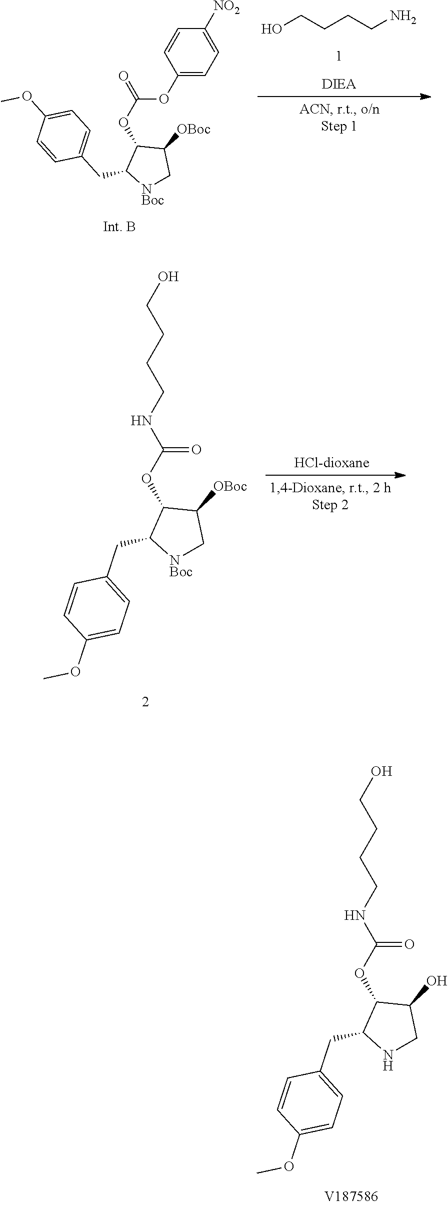

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.01 mmol, 1 eq.) in ACN (16.0 mL) were added DIEA (52.7 mg, 0.40 mmol, 3 eq.) and 4-aminobutan-1-ol (24.2 mg, 0.27 mmol, 2 eq.) at 0° C. The reaction was stirred at r.t. for overnight. After completion of the reaction monitored by LCMS, The reaction mixture was quenched with water and extracted with EA. The extracts were washed with brine, dried over anhydrous sodium sulfate, filtered and concentrated. The residue was purified by Prep-HPLC (10 mmol/L NH₄HCO₃ in water and acetonitrile) to afford tert-butyl (2R,3S,4S)-4-[(tert-butoxycarbonyl)oxy]-3-{[(4-hydroxybutyl)carbamoyl]oxy}-2-[(4-methoxyphenyl)methyl]pyrrolidine-1-carboxylate (70 mg, 95.6%) as a white solid. MS: m/z: Calc'd for C₂₇H₄₂N₂O₉ [M+H−100−56]⁺383. Found, 383.

Step 2: To a stirred solution of tert-butyl (2R,3S,4S)-4-[(tert-butoxycarbonyl)oxy]-3-{1[(4-hydroxybutyl)carbamoyl]oxy}-2-[(4-methoxyphenyl)methyl]pyrrolidine-1-carboxylate (60 mg, 0.11 mmol, 1 eq.) in 1,4-dioxane (1 mL) were added HCl-dioxane (4M, 1 mL) at 0° C. The reaction was stirred at r.t. for 2 h. The resulting mixture was concentrated under reduced pressure. The crude product (60 mg) was purified by Prep-HPLC to afford (2R,3S,4S)-4-hydroxy-2-[(4-methoxyphenyl)methyl]pyrrolidin-3-yl N-(4-hydroxybutyl)carbamate (19.4 mg, 50.3%) as a yellow oil. MS: m/z: Calc'd for C₁₇H₂₆N₂O₅[M+H]⁺339. Found, 339. ¹H NMR (400 MHz, Methanol-d₄) δ 7.23 (d, J=8.4 Hz, 2H), 6.97-6.89 (m, 2H), 4.38 (d, J=4.3 Hz, 1H), 4.20-4.11 (m, 1H), 3.80 (s, 3H), 3.65-3.51 (m, 3H), 3.28-3.06 (m, 3H), 3.10 (s, 1H), 2.96-2.92 (m, 1H), 1.62-1.54 (m, 5H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 24% B in 10 min; Wave Length: 254 nm/220 nm; RT1(min): 8.68

### Synthetic Procedure of V187587

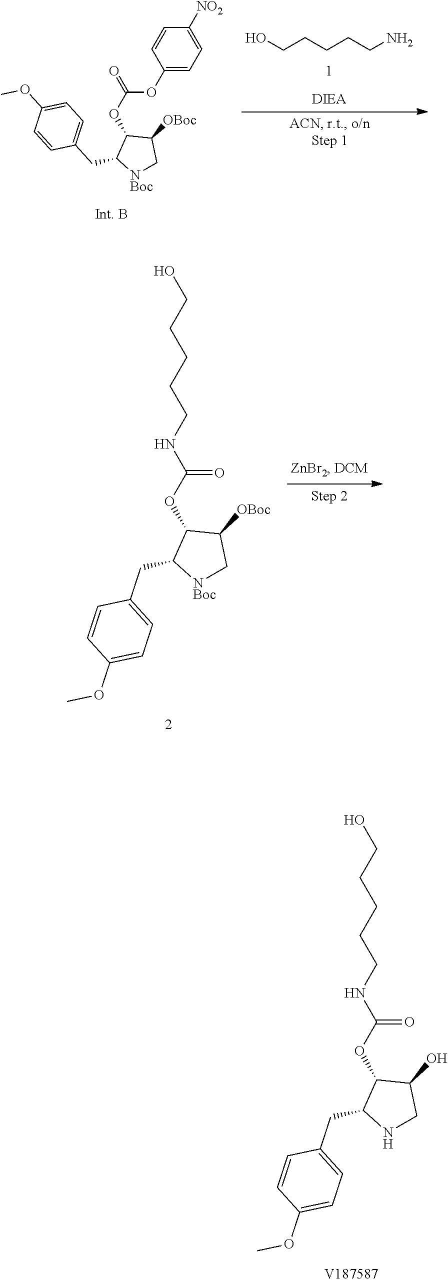

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.01 mmol, 1 eq.) in ACN (3 mL) were added DIEA (52.70 mg, 0.40 mmol, 3 eq.) and 5-aminopentanol (28.0 mg, 0.27 mmol, 2 eq.) at 0° C. The reaction was stirred at r.t. for overnight. After completion of the reaction monitored by LCMS, The reaction mixture was quenched with water and extracted with EA. The extracts were washed with brine, dried over anhydrous sodium sulfate, filtered and concentrated. The residue was purified by Prep-HPLC to obtained tert-butyl (2R,3S,4S)-4-[(tert-butoxycarbonyl)oxy]-3-{[(5-hydroxypentyl)carbamoyl]oxy}-2-[(4-methoxyphenyl)methyl]pyrrolidine-1-carboxylate (70 mg, 90.5%) as a white solid. MS: m/z: Calc'd for C₂₈H₄₄N₂O₉[M+H−100−56]⁺397. Found, 397.

Step 2: To a stirred solution of tert-butyl (2R,3S,4S)-4-[(tert-butoxycarbonyl)oxy]-3-{1[(5-hydroxypentyl)carbamoyl]oxy}-2-[(4-methoxyphenyl)methyl]pyrrolidine-1-carboxylate (60 mg, 0.10 mmol, 1 eq.) in DCM (3 mL) were added ZnBr₂ (73.3 mg, 0.32 mmol, 3 eq.) at 0° C. The reaction was stirred at r.t. for 2 h. The resulting mixture was concentrated under reduced pressure. The crude product (60 mg) was purified by Prep-HPLC to afford (2R,3S,4S)-4-hydroxy-2-[(4-methoxyphenyl)methyl]pyrrolidin-3-yl N-(5-hydroxypentyl)carbamate (18.1 mg, 47.1%) as a white solid. MS: m/z: Calc'd for C₁₈H₂₈N₂O₅[M+H]⁺353. Found, 353. ¹H NMR (400 MHz, Methanol-d₄) δ 7.27-7.19 (m, 2H), 6.97-6.90 (m, 2H), 4.93 (d, J=3.6 Hz, 1H), 4.46-4.35 (m, 1H), 4.16-4.11 (m, 1H), 3.80 (s, 3H), 3.57-3.54 (m, 3H), 3.25-3.07 (m, 4H), 2.97-2.94 (m, 1H), 1.58-1.55 (m, 4H), 1.52-1.38 (m, 2H). Prep-HPLC-conditions: Column: XBridge Prep OBD C18 Column, 30*150 mm, 5 μm; Mobile Phase A: Water (10 nmol/LNH4HCO3), Mobile Phase B: ACN; Flow rate: 60 mL/min mL/min; Gradient: 1% B to 31% B in 10 min; Wave Length: 254 nm/220 nm; RT1(min): 8.5

### Synthetic Procedure of V187588

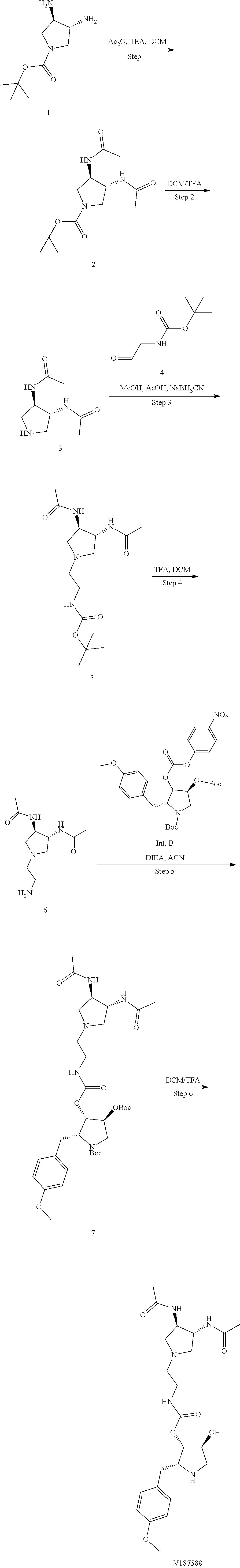

Step 1: To a stirred solution of tert-butyl (3R,4R)-3,4-diaminopyrrolidine-1-carboxylate (100 mg, 0.50 mmol, 1 eq.) and TEA (151 mg, 1.49 mmol, 3 eq.) in DCM (5 mL) was added a solution of Ac₂O (152 mg, 1.49 mmol, 3 eq.) in 1 mL DCM dropwise at 0° C. The resulting reaction mixture was stirred at room temperature for overnight. After completion of the reaction monitored by LCMS, the resulting mixture was concentrated under reduced pressure. The residue was purified by reversed-phase column chromatography (10 mmol/L FA in water and acetonitrile) to afford tert-butyl (3R,4R)-3,4-diacetamidopyrrolidine-1-carboxylate (106 mg, 74.7%) as a colorless oil. MS: m/z: Calc'd for C₁₃H₂₃N₃O₄ [M+H]⁺286. Found, 286.

Step 2: To a stirred solution of tert-butyl (3R,4R)-3,4-diacetamidopyrrolidine-1-carboxylate (106 mg, 0.37 mmol) in DCM (6 mL) were added TFA (3 m) at room temperature. The resulting mixture was stirred for 1 h at room temperature. After completion of the reaction monitored by LCMS, the resulting mixture was concentrated under reduced pressure to afford crude product as a light yellow oil. MS: m/z: Calc'd for C₈H₁₅N₃O₂ [M+H]⁺186. Found, 186.

Step 3: To a stirred solution of N-[(3R,4R)-4-acetamidopyrrolidin-3-yl]acetamide (70 mg, 0.38 mmol, 1 eq.) and tert-butyl N-(2-oxoethyl)carbamate (120 mg, 0.76 mmol, 2 eq.) in MeOH (3 mL) was added AcOH (45 mg, 0.76 mmol, 2 eq.) dropwise. The resulting mixture was stirred for 2 h at room temperature. STAB (160 mg, 0.76 mmol, 2 eq.) was added to the mixture, the resulting mixture was stirred for overnight. After completion of the reaction monitored by LCMS, the resulting mixture was purified directly by reversed-phase column chromatography (10 mmol/L TFA in water and acetonitrile) to afford tert-butyl N-{2-[(3R,4R)-3,4-diacetamido pyrrolidin-1-yl]ethyl}carbamate (60 mg, 48.3%) as a colorless oil. MS: m/z: Calc'd for C₁₅H₂₈N₄O₄ [M+H]⁺329. Found, 329.

Step 4: To a stirred solution of tert-butyl N-{2-[(3R,4R)-3,4-diacetamidopyrrolidin-1-yl]ethyl}carbamate (60 mg, 0.183 mmol) in DCM (5 mL) were added TFA (2 mL) at room temperature. The resulting mixture was stirred for 1 h at room temperature. After completion of the reaction monitored by LCMS, The resulting mixture was concentrated under reduced pressure to afford N-[(3R,4R)-1-(2-aminoethyl)-4-acetamidopyrrolidin-3-yl]acetamide (41 mg, 98.3%) as a yellow oil. MS: m/z: Calc'd for C₁₀H₂₀N₄O₂ [M+H]⁺229. Found, 229.

Step 5: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 70 mg, 0.12 mmol, 1 eq.) and N-[(3R,4R)-1-(2-aminoethyl)-4-acetamidopyrrolidin-3-yl]acetamide (30 mg, 0.13 mmol, 1.1 eq.) in ACN (3 mL) were added DIEA (46 mg, 0.36 mmol, 3 eq.). The resulting mixture was stirred overnight at room temperature. After completion of the reaction monitored by LCMS, the resulting mixture was purified directly by reversed-phase column chromatography (10 mmol/L NH₄HCO₃ in water and acetonitrile) to afford tert-butyl (2R,3S,4S)-4-[(tert-butoxycarbonyl) oxy]-3-[({2-[(3R,4R)-3,4-diacetamidopyrrolidin-1-yl]ethyl}carbamoyl)oxy]-2-[(4-methoxyphenyl) methyl]pyrrolidine-1-carboxylate (32 mg, 39.7%) as a colorless oil. MS: m/z: Calc'd for C₃₃H₅₁N₅O₁₀ [M+H]⁺678. Found, 678.

Step 6: To a stirred solution of tert-butyl (2R,3S,4S)-4-[(tert-butoxycarbonyl)oxy]-3-[({2-[(3R,4R)-3,4-diacetamidopyrrolidin-1-yl]ethyl}carbamoyl)oxy]-2-[(4-methoxyphenyl) methyl]pyrrolidine-1-carboxylate (32 mg, 0.05 mmol) in DCM (4 mL) was added TFA (4 mL) at room temperature. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl N-{2-[(3R,4R)-3,4-diacetamidopyrrolidin-1-yl]ethyl}carbamate; trifluoroacetic acid (14.9 mg, 53.3%) as a white solid. MS: m/z: Calc'd for C₂₃H₃₅N₅O₆ [M+H]⁺478. Found, 478. ¹H NMR (400 MHz, Methanol-d₄) δ 7.29-7.21 (m, 2H), 6.98-6.90 (m, 2H), 5.05-4.99 (m, 1H), 4.52-4.46 (m, 1H), 4.31 (s, 2H), 4.18-4.13 (m, 1H), 3.89 (s, 2H), 3.80 (s, 3H), 3.68-3.49 (m, 4H), 3.49-3.34 (m, 3H), 3.22 (d, J=12.6 Hz, 1H), 3.15 (dd, J=14.3, 6.6 Hz, 1H), 2.99 (dd, J=14.3, 8.9 Hz, 1H), 2.05 (s, 1H), 1.98 (s, 6H). Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 17% B in 10 min; Wave Length: 254 nm/220 nm; RT1(min): 8.68.

### Synthetic Procedure of V187589

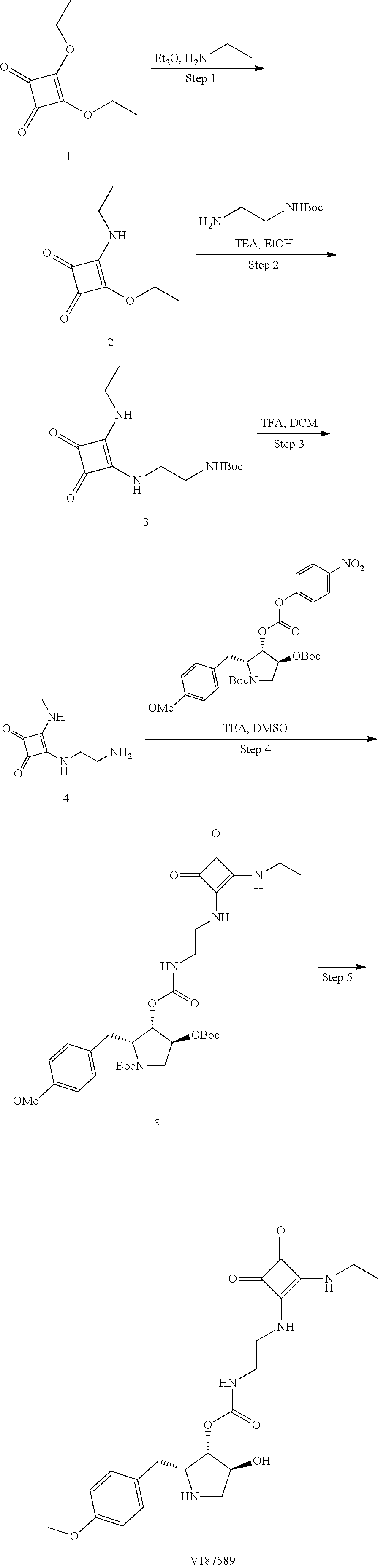

Step 1: To a stirred solution of diethoxycyclobut-3-ene-1,2-dione (300 mg, 1.76 mmol, 1 eq.) in EtOH (10 mL) was added ethylamine (79.49 mg, 1.76 mmol, 1 eq.) at room temperature, and the resulting mixture was stirred for overnight. LCMS showed the reaction was completed. Then the mixture was filtered, washed with EtOH. The filter cake was dried in vacuo to obtain 3-ethoxy-4-(ethylamino)cyclobut-3-ene-1,2-dione (250 mg, 83.8%) as a yellow solid. MS: m/z: Calc'd for C₈H₁₁NO₃ [M+H]⁺170. found N/A.

Step 2: To a stirred solution of 3-ethoxy-4-(ethylamino)cyclobut-3-ene-1,2-dione (250 mg, 1.48 mmol, 1 eq.) and tert-butyl N-(2-aminoethyl)carbamate (355.1 mg, 2.21 mmol, 1.5 eq.) in EtOH (10 mL) was added TEA (448.6 mg, 4.43 mmol, 3 eq.), and the resulting mixture was stirred at room temperature for overnight. LCMS showed the reaction was completed. The mixture was filtered, washed with EtOH. The filter cake was dried in vacuo to obtain tert-butyl N-(2-{[2-(ethylamino)-3,4-dioxocyclobut-1-en-1-yl]amino}ethyl)carbamate (210 mg, 50.1%) as a white solid. MS: m/z: Calc'd for C₁₃H₂₁N₃O₄[M+H−56]⁺228. found 228.

Step 3: To a stirred solution of tert-butyl N-(2-{[2-(ethylamino)-3,4-dioxocyclobut-1-en-1-yl]amino}ethyl)carbamate (210 mg, 0.74 mmol, 1 eq.) in DCM (5 mL) was added TFA (1 mL) and the resulting mixture was stirred at room temperature for 2 h. LCMS showed the reaction was completed. Then the mixture was concentrated to obtain 3-[(2-aminoethyl)amino]-4-(ethylamino)cyclobut-3-ene-1,2-dione (130 mg, 95.7%) as a yellow oil. MS: m/z: Calc'd for C₈H₁₃N₃O₂ [M+H]⁺184. found 184.

Step 4: To a stirred solution of 3-[(2-aminoethyl)amino]-4-(ethylamino)cyclobut-3-ene-1,2-dione (130 mg, 0.71 mmol, 1 eq.) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int B, 417.6 mg, 0.71 mmol, 1 eq.) in DMSO (10 mL) was added DIEA (275.1 mg, 2.13 mmol, 3 eq.), and the resulting mixture was stirred at room temperature for overnight. After completion of the reaction monitored by LCMS, the mixture was purified by reversed-phase column to obtain tert-butyl (2R,3S,4S)-4-[(tert-butoxycarbonyl)oxy]-3-{[(2-{[2-(ethylamino)-3,4-dioxocyclobut-1-en-1-yl]amino}ethyl)carbamoyl]oxy}-2-[(4-methoxyphenyl)methyl]pyrrolidine-1-carboxylate (130 mg, 28.9%) as a yellow solid. MS: m/z: Calc'd for C₃₁H₄₄N₄O₁₀ [M+H]⁺633. found 633.

Step 5: To a stirred solution of tert-butyl (2R,3S,4S)-4-[(tert-butoxycarbonyl)oxy]-3-{[(2-{[2-(ethylamino)-3,4-dioxocyclobut-1-en-1-yl]amino}ethyl)carbamoyl]oxy}-2-[(4-methoxyphenyl) methyl]pyrrolidine-1-carboxylate (120 mg, 0.19 mmol, 1 eq.) in DCM (5 mL) was added TFA (1 mL), and the resulting mixture was stirred at room temperature for 2 h. After completion of the reaction monitored by LCMS, the mixture was concentrated. The residue was purified by Prep-HPLC to obtain (2R,3S,4S)-4-hydroxy-2-[(4-methoxyphenyl)methyl]pyrrolidin-3-yl N-(2-{[2-(ethylamino)-3,4-dioxocyclobut-1-en-1-yl]amino}ethyl)carbamate; trifluoroacetic acid (27.4 mg, 26.1%) as a white solid. MS: m/z: Calc'd for C₂₁H₂₈N₄O₆ [M+H]⁺433. found 433. ¹H NMR (400 MHz, Methanol-d₄) δ 7.28-7.18 (m, 2H), 6.96-6.89 (m, 2H), 4.40 (d, J=4.1 Hz, 1H), 4.17-4.08 (m, 1H), 3.79-3.50 (m, 8H), 3.45 (dd, J=14.0, 5.2 Hz, 1H), 3.33-3.30 (m, 2H), 3.18 (d, J=12.7 Hz, 1H), 3.08 (dd, J=14.2, 6.9 Hz, 1H), 2.92 (dd, J=13.9, 8.7 Hz, 1H), 1.24 (t, J=7.2 Hz, 3H). Prep-HPLC-conditions: Column: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 5% B to 35% B in 10 min; Wave Length: 254 nm/220 nm; RT1(min): 8.68

### Synthetic Procedure of V187591

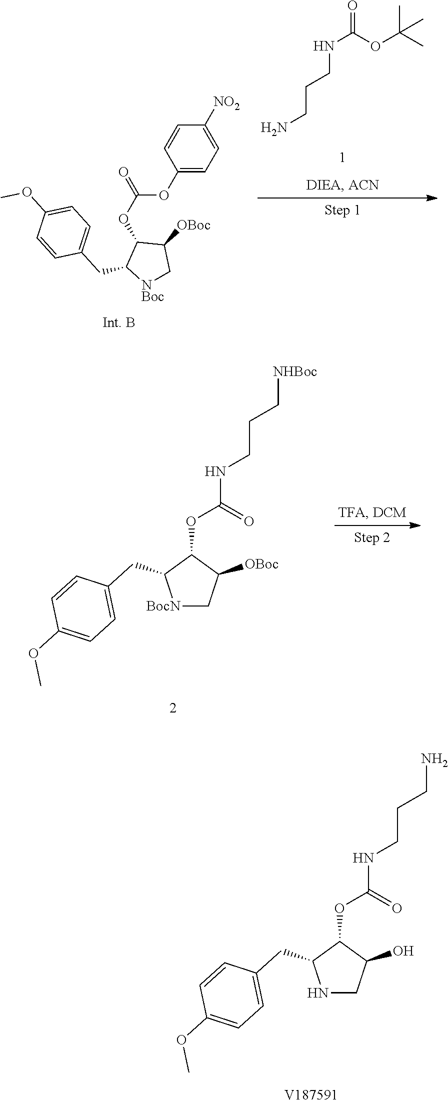

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq.) and tert-butyl N-(3-aminopropyl)carbamate (59 mg, 0.34 mmol, 2 eq.) in ACN (3 mL) was added DIEA (66 mg, 0.51 mmol, 3 eq.). The resulting reaction mixture was stirred at ambient temperature for 3 h. After completion of the reaction monitored by LCMS, the mixture was purified directly by reversed-phase column chromatography (10 mmol/L NH₄HCO₃ in water and acetonitrile) to afford tert-butyl (2R,3S,4S)-3-[({3-[(tert-butoxycarbonyl) amino]propyl}carbamoyl) oxy]-4-[(tert-butoxycarbonyl) oxy]-2-[(4-methoxyphenyl) methyl]pyrrolidine-1-carboxylate (99 mg, 93.4%) as a white solid. MS: m/z: Calc'd for C₃₁H₄₉N₃O₁₀ [M+H−100]⁺524. Found, 524.

Step 2: To a stirred solution of tert-butyl (3S)-4-benzyl-3-{2-[{[(2R,3S,4S)-1-(tert-butoxycarbonyl)-4-[(tert-butoxycarbonyl) oxy]-2-[(4-methoxyphenyl) methyl]pyrrolidin-3-yl]oxy}carbonyl) amino]ethyl}piperazine-1-carboxylate (75 mg, 0.10 mmol) in DCM (5 ml) was added TFA (5 mL) at room temperature. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl N-{2-[(2S)-1-benzylpiperazin-2-yl]ethyl}carbamate (14.5 mg, 31.2%) as a white solid. MS: m/z: Calc'd for C₁₆H₂₅N₃O₄ [M+H]⁺324. Found, 324. ¹H NMR (400 MHz, Methanol-d₄) δ 7.28-7.21 (m, 2H), 6.97-6.89 (m, 2H), 4.96 (d, J=4.2 Hz, 1H), 4.39 (d, J=4.2 Hz, 1H), 4.17-4.13 (m, 1H), 3.80 (s, 3H), 3.59 (dd, J=12.6, 4.3 Hz, 1H), 3.33-3.18 (m, 3H), 3.11 (dd, J=14.1, 7.2 Hz, 1H), 3.05-2.94 (m, 3H), 1.91-1.85 (m, 2H). Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min mL/min; Gradient: 16% B to 46% B in 7 min; Wave Length: 254 nm/220 nm; RT1(min): 6.6.

### Synthetic Procedure of V187592

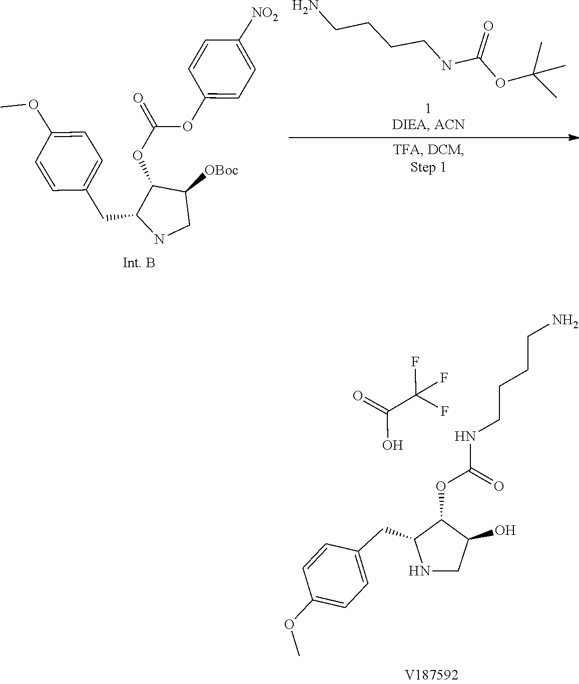

To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1 eq) and tert-butyl N-(4-aminobutyl)carbamate (64 mg, 0.34 mmol, 2 eq) in ACN (5 mL) was added DIEA (66 mg, 0.51 mmol, 3 eq). The resulting reaction mixture was stirred at room temperature for 3 h. After completion of the reaction monitored by LCMS, the mixture was concentrated under reduced pressure. To the residue was added DCM (5 mL) and TFA (5 mL) dropwise 0° C. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-(4-aminobutyl)carbamate; trifluoroacetic acid (14.1 mg, 18.3%) as a white solid. MS: m/z: Calc'd for C₁₇H₂₇N₃O₄ [M+H]⁺338. found 338. ¹H NMR (400 MHz, DMSO-d₆) δ 9.73 (s, 1H), 9.31 (s, 1H), 7.87 (s, 3H), 7.40-7.38 (m, 1H), 7.21 (t, J=6.6 Hz, 2H), 6.90 (d, J=6.7 Hz, 2H), 6.02-5.97 (m, 1H), 4.72 (d, J=4.2 Hz, 1H), 4.22 (d, J=4.2 Hz, 1H), 3.98 (s, 1H), 3.74-3.72 (m, 3H), 3.36 (s, 1H), 3.16-2.86 (m, 6H), 2.82-2.80 (m, 2H), 1.53-1.50 (m, 4H).

Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 17% B in 10 min; Wave Length: 254 nm/220 nm; RT1(min): 8.68.

### Synthetic Procedure of V187593

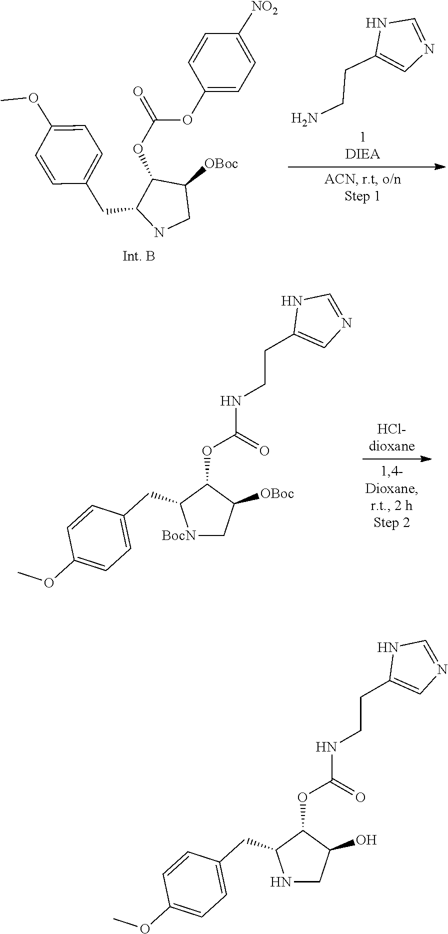

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.01 mmol, 1 eq.) in ACN (3 mL) were added DIEA (52.7 mg, 0.40 mmol, 3 eq.) and 1H-imidazole-5-ethanamine (30.2 mg, 0.27 mmol, 2 eq.) at 0° C. The reaction was stirred at r.t. for overnight. After completion of the reaction monitored by LCMS. The reaction mixture was quenched with water and extracted with EA. The extracts were washed with brine, dried over anhydrous sodium sulfate, filtered and concentrated. The residue was purified by Prep-HPLC (10 mmol/L NH₄HCO₃ in water and acetonitrile) to afford tert-butyl (2R,3S,4S)-4-[(tert-butoxycarbonyl)oxy]-3-({[2-(3H-imidazol-4-yl)ethyl]carbamoyl}oxy)-2-[(4-methoxyphenyl) methyl]pyrrolidine-1-carboxylate (34 mg, 44.6%) as a white solid. MS: m/z: Calc'd for C₂₈H₄₀N₄O₈ [M+H]⁺561. Found, 561.

Step 2: To a stirred solution of tert-butyl (2R,3S,4S)-4-[(tert-butoxycarbonyl)oxy]-3-({[2-(3H-imidazol-4-yl)ethyl]carbamoyl}oxy)-2-[(4-methoxyphenyl)methyl]pyrrolidine-1-carboxylate (30 mg, 0.05 mmol, 1 eq.) and in 1,4-dioxane (1.5 mL) were added HCl-dioxane (1.5 mL) at 0° C. The reaction was stirred at r.t. for 2 h. The resulting mixture was concentrated under reduced pressure. The crude product (30 mg) was purified by Prep-HPLC to afford (2R,3S,4S)-4-hydroxy-2-[(4-methoxyphenyl)methyl]pyrrolidin-3-yl N-[2-(3H-imidazol-4-yl)ethyl]carbamate (20.2 mg, 99.5%) as a light yellow oil. MS: m/z: Calc'd for C₁₈H₂₄N₄O₄ [M+H]⁺361. Found, 361. ¹H NMR (400 MHz, Methanol-d4) δ 8.83 (d, J=1.4 Hz, 1H), 7.40 (s, 1H), 7.28-7.18 (m, 2H), 6.96-6.88 (m, 2H), 4.93 (d, J=3.4 Hz, 1H), 4.34 (d, J=4.2 Hz, 1H), 4.19-4.09 (m, 1H), 3.80 (s, 3H), 3.57-3.52 (m, 1H), 3.50-3.45 (m, 2H), 3.20 (d, J=12.6 Hz, 1H), 3.07-3.04 (m, 1H), 2.99-2.89 (m, 3H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 17% B in 10 min; Wave Length: 254 nm/220 nm; RT1(min): 8.68

### Synthetic Procedure of V187597

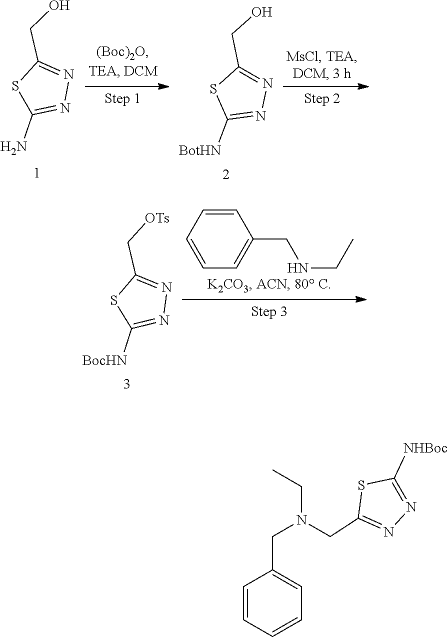

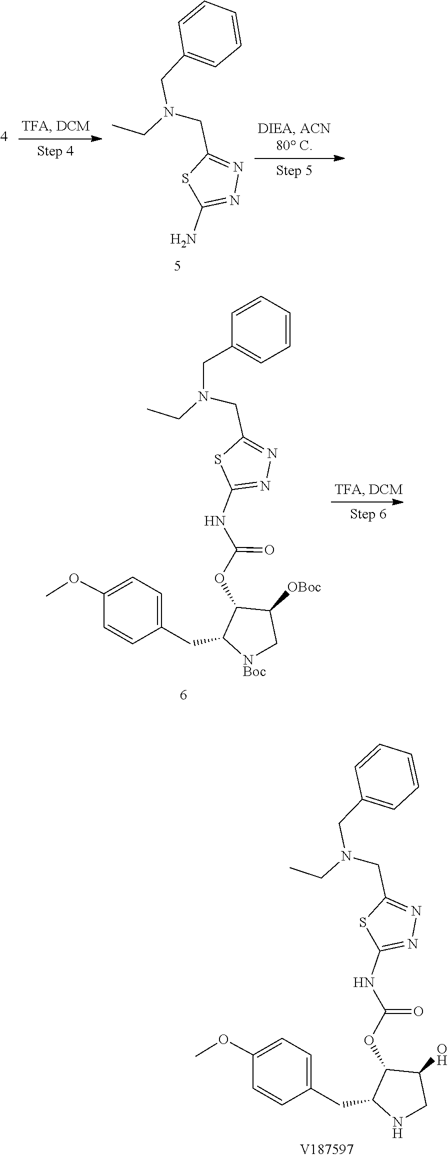

Step 1: To a stirred solution of (5-amino-1,3,4-thiadiazol-2-yl)methanol (500 mg, 3.81 mmol, 1 eq.) and di-tert-butyl dicarbonate (1664 mg, 7.62 mmol, 2 eq.) in DCM (100 mL) was added TEA (1157.3 mg, 11.43 mmol, 3 eq.) at 0° C. The resulting mixture was stirred at room temperature for overnight. The reaction mixture was quenched with water and extracted with EA. The extracts were washed with brine, dried over anhydrous sodium sulfate, filtered and concentrated. The residue was purified by Prep-TLC PE/EA (1:9) to afford tert-butyl N-[5-(hydroxymethyl)-1,3,4-thiadiazol-2-yl]carbamate (150 mg, 17.0%) as a white solid. MS: m/z: Calc'd for C₈H₁₃N₃O₃S [M+H]⁺232. found 232.

Step 2: To a stirred mixture of tert-butyl N-[5-(hydroxymethyl)-1,3,4-thiadiazol-2-yl]carbamate (200 mg, 0.86 mmol, 1 eq.) in DCM (5 mL) was added Et₃N (262.5 mg, 2.59 mmol, 3 eq.) and TsCl (197.8 mg, 1.03 mmol, 1.2 eq.) at 0° C. The resulting mixture was stirred at room temperature for 3 h. The reaction mixture was quenched with water and extracted with EA. The extracts were washed with brine, dried over anhydrous sodium sulfate, filtered and concentrated. The residue was purified by Prep-TLC to obtained tert-butyl N-(5-{[(4-methylbenzenesulfonyl) oxy]methyl}-1,3,4-thiadiazol-2-yl)carbamate (250 mg, 75.0%) as a yellow solid. MS: m/z: Calc'd for C₁₅H₁₉N₃O₅S₂[M+H−56]⁺330. found 330.

Step 3: To a stirred mixture of tert-butyl N-(5-{[(4-methylbenzenesulfonyl)oxy]methyl}-1,3,4-thiadiazol-2-yl)carbamate (500 mg, 1.297 mmol, 1 eq.) and benzenemethanamine, N-ethyl- (350.7 mg, 2.59 mmol, 2 eq.) in DMF (10 mL) was added K₂CO₃ (537.8 mg, 3.89 mmol, 3 eq.) at r.t. The resulting mixture was stirred at 80° C. for 1 h. The reaction mixture was quenched with water and extracted with EA. The combined extracts were washed with brine, dried over anhydrous sodium sulfate, filtered and concentrated. The residue was purified by Prep-HPLC to obtained tert-butyl N-(5-{[benzyl(ethyl)amino]methyl}-1,3,4-thiadiazol-2-yl)carbamate (500 mg, 94.0%) as a yellow oil. MS: m/z: Calc'd for C₁₇H₂₄N₄O₂S [M+H]⁺349. found 349.

Step 4: To a stirred solution of tert-butyl N-(5-{[benzyl(ethyl)amino]methyl}-1,3,4-thiadiazol-2-yl)carbamate (195 mg, 0.56 mmol, 1 eq.) in DCM (3 mL) were added TFA (1 mL) at 0° C. The reaction was stirred at r.t. for 2 h. After completion of the reaction monitored by LCMS, The resulting mixture was concentrated under reduced pressure. The resulting residue was purified by reverse phase flash to afford 5-{[benzyl(ethyl)amino]methyl}-1,3,4-thiadiazol-2-amine (100 mg, 71.9%) as a white solid. MS: m/z: Calc'd for C₁₂H₁₆N₄S [M+H]⁺249. Found, 249.

Step 5: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (95 mg, 0.01 mmol, 1 eq.) in ACN (3 mL) were added DIEA (62.5 mg, 0.48 mmol, 3 eq.) and 5-{[benzyl (ethyl)amino]methyl}-1,3,4-thiadiazol-2-amine (80.1 mg, 0.32 mmol, 2.0 eq.) at room temperature and the resulting mixture was stirred at 80° C. for overnight. After completion of the reaction monitored by LCMS, The resulting mixture was concentrated under reduced pressure. The residue product was purified by reverse phase flash to afford tert-butyl (2R,3S,4S)-3-{[(5-{[benzyl(ethyl)amino]methyl}-1,3,4-thiadiazol-2-yl)carbamoyl]oxy}-4-[(tert-butoxycarbonyl) oxy]-2-[(4-methoxyphenyl)methyl]pyrrolidine-1-carboxylate (33 mg, 29.3%) as a white solid. MS: m/z: Calc'd for C₃₅H₄₇N₅O₈S[M+H]⁺698. Found, 698.

Step 6: To a stirred solution of tert-butyl (2R,3S,4S)-3-{[(5-{[benzyl(ethyl)amino]methyl}-1,3,4-thiadiazol-2-yl)carbamoyl]oxy}-4-[(tert-butoxycarbonyl)oxy]-2-[(4-methoxyphenyl) methyl]pyrrolidine-1-carboxylate (30 mg, 0.04 mmol, 1 eq.) in DCM (3 mL) were added TFA (1 mL) at 0° C. The reaction was stirred at r.t. for 2 h. The resulting mixture was concentrated under reduced pressure. The crude product (30 mg) was purified by Prep-HPLC to afford (2R,3S,4S)-4-hydroxy-2-[(4-methoxyphenyl)methyl]pyrrolidin-3-yl N-(5-{[benzyl(ethyl)amino]methyl}-1,3,4-thiadiazol-2-yl)carbamate (21.3 mg, 98.1%) as a white solid. MS: m/z: Calc'd for C₃₅H₃₁N₅O₄S[M+H]⁺498. Found, 498. ¹H NMR (400 MHz, Methanol-d₄) δ 7.60-7.50 (m, 5H), 7.30-7.22 (m, 2H), 6.96-6.88 (m, 2H), 5.19-5.14 (m, 1H), 4.65 (s, 2H), 4.56-4.50 (m, 1H), 4.41 (s, 2H), 4.32-4.21 (m, 1H), 3.79 (s, 3H), 3.64-3.52 (m, 1H), 3.34-3.15 (m, 4H), 3.05-3.01 (m, 1H), 1.42 (t, J=7.2 Hz, 3H). Prep-HPLC-conditions: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 29% B in 10 min; Wave Length: 254 nm/220 nm; RT1(min): 8.68

### Synthetic Procedure of V187598

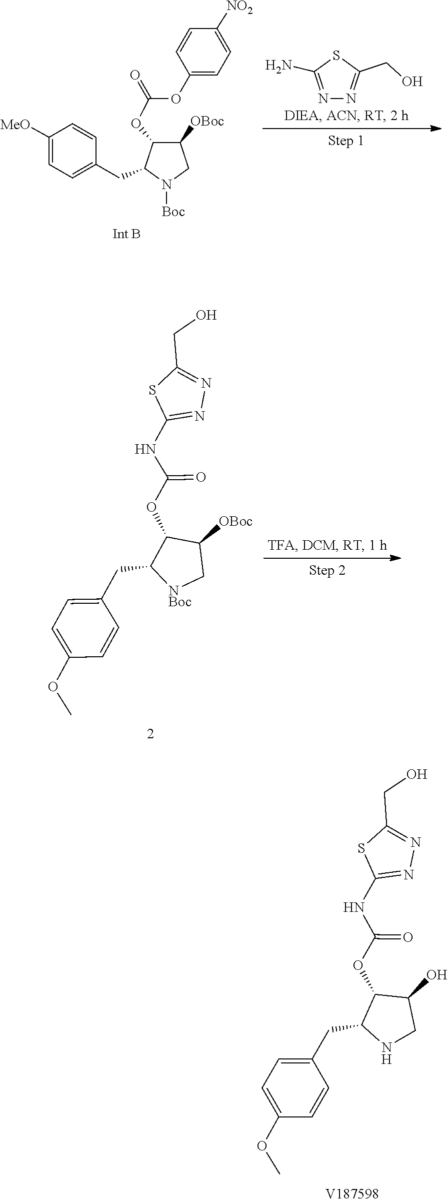

Step 1: To a stirred solution of (5-amino-1,3,4-thiadiazol-2-yl)methanol (45 mg, 0.34 mmol, 2 equiv) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.00 equiv) in ACN (6 mL) was added DIEA (66 mg, 0.51 mmol, 3 equiv) at 0° C. The resulting mixture was stirred for 72 h at room temperature. Desired product could be detected by LCMS. The resulting mixture was concentrated under reduced pressure. The residue was purified by reversed-phase flash chromatography with the following conditions: column, C18 silica gel; mobile phase, MeCN in Water (0.05% TFA), 50% to 70% gradient in 10 min; detector, UV 254 nm. This resulted in tert-butyl (2R,3S,4S)-4-[(tert-butoxycarbonyl)oxy]-3-({[5-(hydroxymethyl)-1,3,4-thiadiazol-2-yl]carbamoyl}oxy)-2-[(4-methoxyphenyl)methyl]pyrrolidine-1-carboxylate (50 mg, 50.6%) as a yellow oil. MS: m/z: Calc'd for C₂₆H₃₆N₄O₉S [M+H]⁺581. found 581.

Step 2: To a stirred solution of tert-butyl (2R,3S,4S)-4-[(tert-butoxycarbonyl)oxy]-3-({[5-(hydroxymethyl)-1,3,4-thiadiazol-2-yl]carbamoyl}oxy)-2-[(4-methoxyphenyl)methyl][0507]pyrrolidine-1-carboxylate (50 mg, 0.08 mmol, 1 equiv) in DCM (5 mL) was added TFA (1 mL) at 0° C. The resulting mixture was stirred for 2 h at room temperature. Desired product could be detected by LCMS. The resulting mixture was concentrated under reduced pressure. The crude product (20 mg) was purified by Prep-HPLC to afford (2R,3S,4S)-4-hydroxy-2-[(4-methoxyphenyl)methyl]pyrrolidin-3-yl N-[5-(hydroxymethyl)-1,3,4-thiadiazol-2-yl]carbamate; trifluoroacetic acid (23.3 mg, 54.2%) as a white solid. MS: m/z: Calc'd for C₁₆H₂₀N₄O₅S [M+H]⁺381. found 381. ¹H NMR (400 MHz, Methanol-d₄) δ 7.24-7.16 (m, 2H), 6.95-6.87 (m, 2H), 5.52-5.30 (m, 2H), 5.05-4.93 (m, 1H), 4.52-4.46 (m, 1H), 4.27-4.18 (m, 1H), 3.80 (s, 3H), 3.62 (dd, J=12.8, 4.5 Hz, 1H), 3.24 (d, J=12.8 Hz, 1H), 3.12 (dd, J=14.1, 7.2 Hz, 1H), 2.99 (dd, J=14.2, 8.6 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 21% B in 10 min; Wave Length: 254 nm/220 nm; RT1(min): 8.68

### Synthetic Procedure of V187620

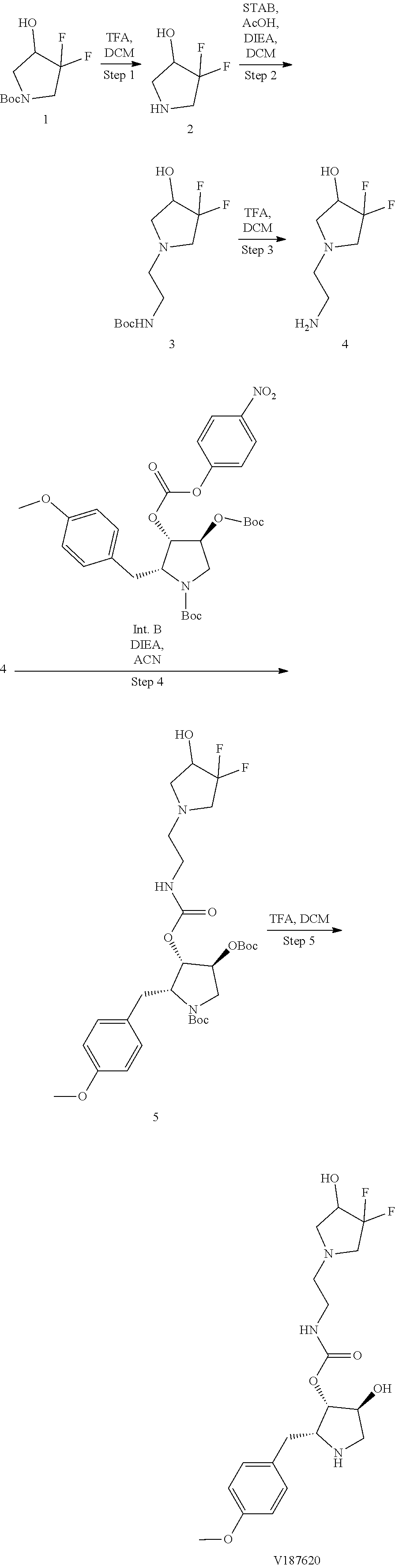

Step 1: To a stirred solution of tert-butyl 3,3-difluoro-4-hydroxypyrrolidine-1-carboxylate (200 mg, 0.89 mmol) in DCM (6 mL) was added TFA (3 mL) at room temperature. The resulting reaction mixture was stirred at room temperature for 1 h. After completion of the reaction monitored by LCMS, the resulting mixture was concentrated under reduced pressure to afford 4,4-difluoropyrrolidin-3-ol (110 mg, 99.7%) as a brown oil.

Step 2: To a stirred solution of 4,4-difluoropyrrolidin-3-ol (110 mg, 0.89 mmol, 1 eq.) and K₂CO₃ (370 mg, 2.68 mmol, 3 eq.) in DMF (3 mL) were added tert-butyl N-(2-bromoethyl)carbamate (240 mg, 1.07 mmol, 1.2 eq.) at room temperature. The resulting mixture was stirred for overnight. After completion of the reaction monitored by LCMS. The resulting mixture was filtered, the filter cake was washed with MeCN (3×10 mg). The filtrate was concentrated under reduced pressure. The resulting mixture was purified by reversed-phase column chromatography (10 mmol/L NH₄HCO₃ in water and acetonitrile) to afford tert-butyl N-[2-(3,3-difluoro-4-hydroxypyrrolidin-1-yl)ethyl]carbamate (78 mg, 32.8%) as a colorless oil. MS: m/z: Calc'd for C₁₁H₂₀F₂N₂O₃[M+H]⁺267. Found, 267.

Step 3: To a stirred solution of tert-butyl N-[2-(3,3-difluoro-4-hydroxypyrrolidin-1-yl)ethyl]carbamate (78 mg, 0.29 mmol) in DCM (4 mL) was added TFA (2 mL) and the resulting reaction mixture was stirred at room temperature for 1 h. After completion of the reaction monitored by LCMS, the resulting mixture was concentrated under reduced pressure to afford crude product as a yellow oil. MS: m/z: Calc'd for C₆H₁₂F₂N₂O [M+H]⁺167. Found, 167.

Step 4: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.14 mmol, 1 eq.) and 1-(2-aminoethyl)-4,4-difluoropyrrolidin-3-ol (45.17 mg, 0.27 mmol, 2 eq.) in ACN (5 mL) were added DIEA (53 mg, 0.41 mmol, 3 eq.). The resulting mixture was stirred overnight at room temperature. After completion of the reaction monitored by LCMS, the resulting mixture was purified directly by reversed-phase column chromatography (10 mmol/L NH₄HCO₃ in water and acetonitrile) to afford tert-butyl (2R,3S,4S)-4-[(tert-butoxycarbonyl)oxy]-3-({[2-(3,3-difluoro-4-hydroxypyrrolidin-1-yl)ethyl]carbamoyl}oxy)-2-[(4-methoxyphenyl) methyl]pyrrolidine-1-carboxylate (78 mg, 93.2%) as a colorless oil. MS: m/z: Calc'd for Chemical Formula: C₂₉H₄₃F₂N₃O₉ [M+H]⁺616. Found, 616.

Step 5: To a stirred solution of tert-butyl (2R,3S,4S)-4-[(tert-butoxycarbonyl)oxy]-3-[({2-[2-(imidazol-1-yl) ethoxy]ethyl}carbamoyl) oxy]-2-[(4-methoxyphenyl) methyl]pyrrolidine-1-carboxylate (82 mg, 0.14 mmol) in DCM (4 mL) was added TFA (4 mL) at room temperature. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl N-{2-[2-(imidazol-1-yl) ethoxy]ethyl}carbamate; trifluoroacetic acid (16.3 mg, 23.0%) as a white solid. MS: m/z: Calc'd for C₁₉H₂₇F₂N₃O₅ [M+H]⁺416. Found, 416. ¹H NMR (400 MHz, Methanol-d₄) δ 7.28-7.21 (m, 2H), 6.97-6.89 (m, 2H), 4.99 (d, J=3.5 Hz, 1H), 4.41 (dd, J=13.7, 5.8 Hz, 2H), 4.17-4.15 (m, 1H), 3.96-3.81 (m, 2H), 3.80-3.78 (m, 3H), 3.75-3.67 (m, 1H), 3.54-3.52 (m, 4H), 3.39-3.33 (m, 2H), 3.21 (d, J=12.7 Hz, 1H), 3.12 (dd, J=14.3, 6.8 Hz, 1H), 2.99 (dd, J=14.2, 8.7 Hz, 1H). Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 17% B in 10 min; Wave Length: 254 nm/220 nm; RT1(min): 8.68

### Synthetic Procedure of V187623

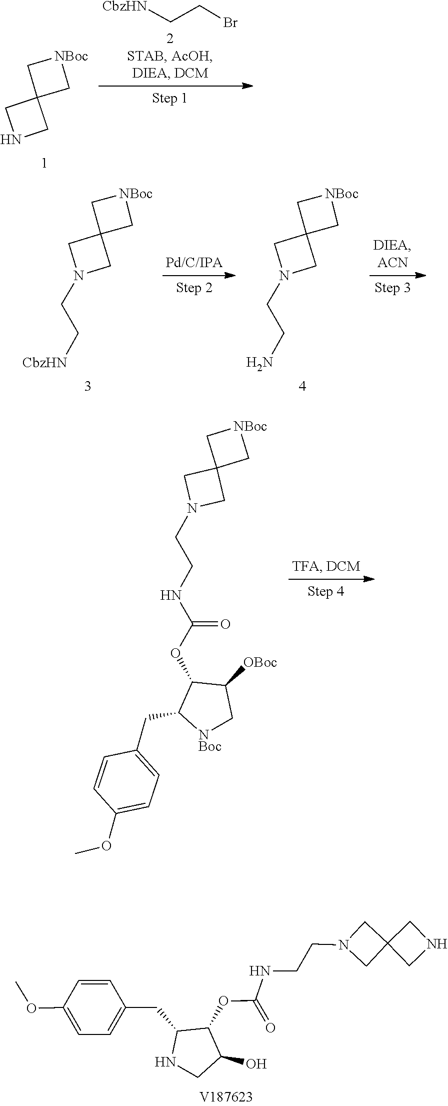

Step 1: To a stirred solution of tert-butyl 2,6-diazaspiro[3.3]heptane-2-carboxylate (300 mg, 1.51 mmol, 1 eq.) in ACN (5 mL) were added K₂CO₃ (627.3 mg, 4.53 mmol, 3 eq.) and benzyl N-(2-bromoethyl)carbamate (781.1 mg, 3.02 mmol, 2 eq.) dropwise at 0° C. The reaction was stirred at r.t. for overnight. After completion of the reaction monitored by LCMS, the resulting mixture was concentrated under reduced pressure. The residue product was purified by reverse phase flash to afford tert-butyl 6-(2-{[(benzyloxy)carbonyl]amino}ethyl)-2,6-diazaspiro [3.3]heptane-2-carboxylate (300 mg, 52.8%) as a white solid. MS: m/z: Calc'd for C₂₀H₂₉N₃O₄[M+H]⁺376. Found, 376.

Step 2: Under a nitrogen atmosphere, Pd/C (125 mg, 1.17 mmol, 1.76 eq.) was added to a solution of tert-butyl 6-(2-{[(benzyloxy)carbonyl]amino}ethyl)-2,6-diazaspiro[3.3]heptane-2-carboxylate (250 mg, 0.66 mmol, 1 eq.) in IPA (5 mL), H₂ was subsequently introduced into the reaction system, and the resulting mixture was stirred at r.t. for 2 h. The reaction mixture was filtered to obtain tert-butyl 6-(2-aminoethyl)-2,6-diazaspiro[3.3]heptane-2-carboxylate (100 mg, 62.2%) as a yellow solid. MS: m/z: Calc'd for C₁₂H₂₃N₃O₂ [M+H]⁺242. Found, 242.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 95 mg, 0.01 mmol, 1 eq.) in ACN (3 mL) were added tert-butyl 6-(2-aminoethyl)-2,6-diazaspiro [3.3]heptane-2-carboxylate (77.9 mg, 0.32 mmol, 2 eq.) and DIEA (62.5 mg, 0.48 mmol, 3 eq.) at 0° C. The reaction was stirred at r.t. for overnight. The reaction mixture was quenched with water and extracted with EA. The extracts were washed with brine, dried over anhydrous sodium sulfate, filtered and concentrated. The residue was purified by Prep-HPLC to afford tert-butyl 6-{2-[({[(2R,3S,4S)-1-(tert-butoxycarbonyl)-4-[(tert-butoxycarbonyl)oxy]-2-[(4-methoxy phenyl) methyl]pyrrolidin-3-yl]oxy}carbonyl)amino]ethyl}-2,6-diazaspiro [3.3]heptane-2-carboxylate (70 mg, 62.7%) as a yellow solid. MS: m/z: Calc'd for C₃₅H₅₄N₄O₁₀[M+H]⁺691. Found, 691.

Step 4: To a stirred solution of tert-butyl 6-{2-[({[(2R,3S,4S)-1-(tert-butoxycarbonyl)-4-[(tert-butoxycarbonyl)oxy]-2-[(4-methoxyphenyl)methyl]pyrrolidin-3-yl]oxy}carbonyl)amino]ethyl}-2,6-diazaspiro[3.3]heptane-2-carboxylate (65 mg, 0.09 mmol, 1 eq.) in DCM (3 mL) were added TFA (1 mL) at 0° C. The reaction was stirred at r.t. for 2 h. The resulting mixture was concentrated under reduced pressure. The crude product (60 mg) was purified by Prep-HPLC to afford (2R,3S,4S)-4-hydroxy-2-[(4-methoxyphenyl)methyl]pyrrolidin-3-yl N-(2-{2,6-diazaspiro [3.3]heptan-2-yl}ethyl)carbamate (26.3 mg, 71.0%) as a light yellow solid. MS: m/z: Calc'd for C₂₀H₃₀N₄O₄[M+H]⁺391. Found, 391. ¹H NMR (400 MHz, Methanol-d₄) δ 7.28-7.20 (m, 2H), 6.98-6.89 (m, 2H), 5.03-4.97 (m, 1H), 4.46 (s, 5H), 4.33 (s, 4H), 4.17-4.11 (m, 1H), 3.80 (s, 3H), 3.56-3.54 (m, 1H), 3.51-3.34 (m, 2H), 3.20 (d, J=12.6 Hz, 2H), 3.11-3.06 (m, 1H), 2.99-2.98 (m, 1H) 2.97-2.93 (m, 1H). Column: Atlantis T3 Prep OBD Column, 19*250 mm, 5 μm; Mobile Phase A: Water (0.1% FA), Mobile Phase B: ACN; Flow rate: 25 mL/min mL/min; Gradient: 3% B to 25% B in 8 min; Wave Length: 254 nm/220 nm; RT1(min): 7.32

### Synthetic Procedure of V187624

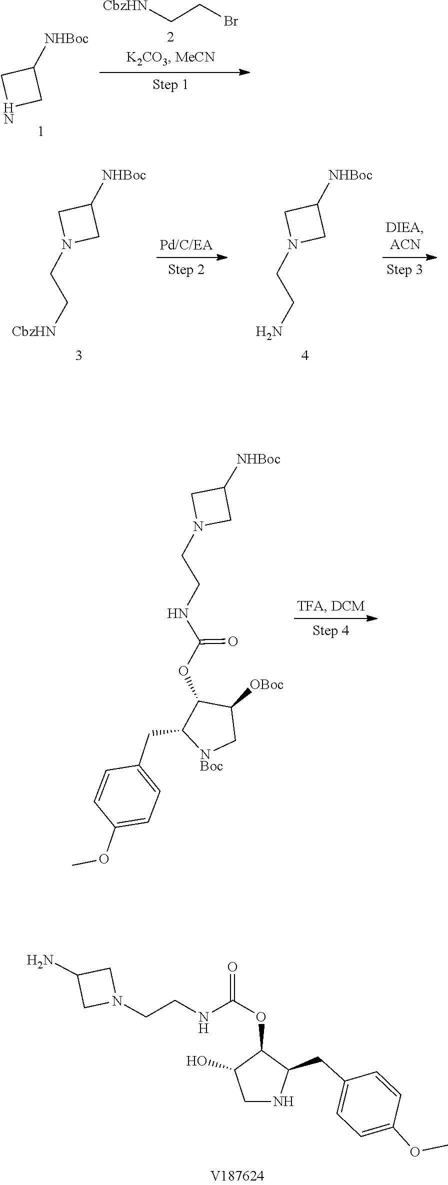

Step 1: To a stirred solution of tert-butyl N-(azetidin-3-yl)carbamate (300 mg, 1.74 mmol, 1 eq.) in ACN (5 mL) were added K₂CO₃ (722.2 mg, 5.22 mmol, 3 eq.) and benzyl N-(2-bromoethyl)carbamate (449.6 mg, 1.74 mmol, 1 eq.) at r.t. The reaction was stirred at r.t. for overnight. The reaction mixture was quenched with water and extracted with EA. The extracts were washed with brine, dried over anhydrous sodium sulfate, filtered and concentrated. The residue was purified by Prep-HPLC (10 mmol/L NH₄HCO₃ in water and acetonitrile) to afford benzyl N-(2-{3-[(tert-butoxycarbonyl)amino]azetidin-1-yl}ethyl)carbamate (320 mg, 52.5%) as a white solid. MS: m/z: Calc'd for C1₈H₂₇N₃O₄[M+H]⁺350. Found, 350.

Step 2: Under a nitrogen atmosphere, Pd/C (150 mg, 1.41 mmol) was added to a solution of benzyl N-(2-{3-[(tert-butoxycarbonyl)amino]azetidin-1-yl}ethyl)carbamate (310 mg, 0.88 mmol, 1 eq.) in EA (5 mL), H₂ was subsequently introduced into the reaction system, and the resulting mixture was stirred at r.t. for 2 h. The reaction mixture was filtered to obtain tert-butyl N-[1-(2-aminoethyl)azetidin-3-yl]carbamate (220 mg, 100%) as a yellow solid. MS: m/z: Calc'd for C₁₀H₂₁N₃O₂ [M+H]⁺216. Found, 216.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.01 mmol, 1 eq.) in ACN (3 mL) were added DIEA (65.8 mg, 0.51 mmol, 3 eq.) and tert-butyl N-[1-(2-aminoethyl)azetidin-3-yl]carbamate (36.5 mg, 0.17 mmol, 1 eq.) at room temperature and the resulting mixture was stirred for overnight. The reaction mixture was quenched with water and extracted with EA. The extracts were washed with brine, dried over anhydrous sodium sulfate, filtered and concentrated. The residue was purified by Prep-HPLC to afford tert-butyl (2R,3S,4S)-3-{[(2-{3-[(tert-butoxycarbonyl)amino]azetidin-1-yl}ethyl) carbamoyl]oxy}-4-[(tert-butoxycarbonyl)oxy]-2-[(4-methoxyphenyl) methyl]pyrrolidine-1-carboxylate (75 mg, 66.4%) as a yellow solid. MS: m/z: Calc'd for C₃₃H₂₂N₄O₁₀[M+H]⁺665. Found, 665.

Step 4: To a stirred solution of tert-butyl (2R,3S,4S)-3-{[(2-{3-[(tert-butoxycarbonyl) amino]azetidin-1-yl}ethyl)carbamoyl]oxy}-4-[(tert-butoxycarbonyl)oxy]-2-[(4-methoxyphenyl)methyl]pyrrolidine-1-carboxylate (70 mg, 0.10 mmol, 1 eq.) in DCM (3 mL) were added TFA (1 mL) at 0° C. The reaction was stirred at r.t. for 2 h. The resulting mixture was concentrated under reduced pressure. The crude product (70 mg) was purified by Prep-HPLC to afford (2R,3S,4S)-4-hydroxy-2-[(4-methoxyphenyl)methyl]pyrrolidin-3-yl N-[2-(3-aminoazetidin-1-yl)ethyl]carbamate (19.3 mg, 50.0%) as a light yellow solid. MS: m/z: Calc'd for C₁₈H₂₈N₄O₄ [M+H]⁺365. Found, 365. ¹H NMR (400 MHz, Methanol-d4) δ 7.28-7.21 (m, 2H), 6.97-6.89 (m, 2H), 4.99 (d, J=3.5 Hz, 1H), 4.57-4.49 (m, 2H), 4.45 (d, J=4.1 Hz, 1H), 4.36 (td, J=10.5, 6.0 Hz, 3H), 4.17 (m, 1H), 3.80 (s, 3H), 3.61-3.50 (m, 1H), 3.53-3.36 (m, 4H), 3.20 (d, J=12.6 Hz, 1H), 3.12 (m, 1H), 2.97 (m, 1H). Column: Welch Ultimate XB-C18 50*250, 10 μm; Mobile Phase A: Water (0.05% TFA), Mobile Phase B: ACN; Flow rate: 60 mL/min mL/min; Gradient: 1% B to 30% B in 20 min; Wave Length: 254 nm/220 nm; RT1(min): 13.5

### Synthetic Procedure of V187625

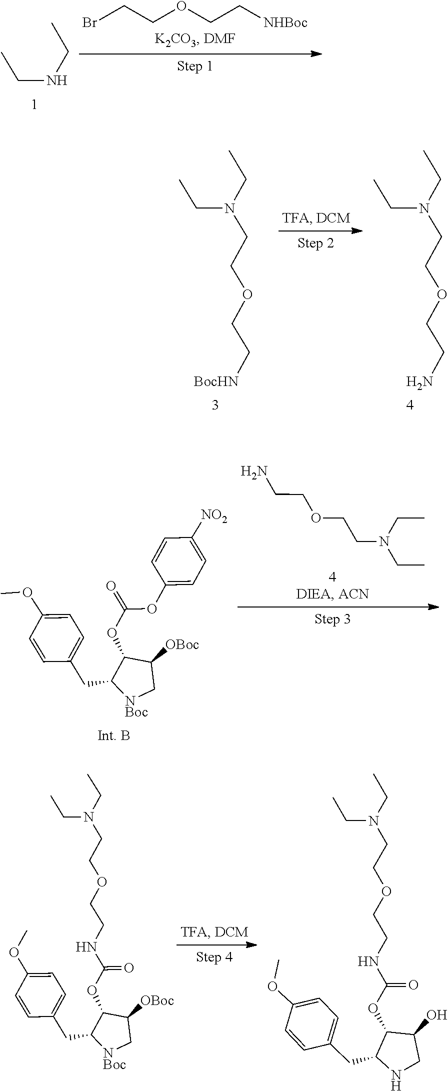

Step 1: To a stirred solution of diethylamine (300 mg, 4.10 mmol, 1 eq.) and tert-butyl N-[2-(2-bromoethoxy)ethyl]carbamate (989.9 mg, 3.69 mmol, 0.9 eq.) in DMF (10 mL) was added K₂CO₃ (1.70 g, 12.31 mmol, 3 eq.) at 80° C. under nitrogen atmosphere. The resulting mixture was stirred at 80° C. for 12h. Upon completion, the reaction mixture was concentrated in vacuo. The residue was purified by reversed-phase flash chromatography with the following conditions: column, C18 silica gel; mobile phase, ACN in Water (0.5% TFA), 10% to 50% gradient in 20 min; detector, UV 254 nm. This resulted in tert-butyl N-{2-[2-(diethylamino)ethoxy]ethyl}carbamate (1 g, 93.6%) as a light yellow oil. MS: m/z: Calc'd for C₁₃H₂₈N₂O₃ [M+H]⁺261. Found, 261.

Step 2: To a stirred solution of tert-butyl N-{2-[2-(diethylamino)ethoxy]ethyl}carbamate (400 mg, 1.54 mmol) in DCM (5 mL) was added TFA (1 mL) dropwise at 0° C. under nitrogen atmosphere. The resulting mixture was stirred at room temperature for 1h. After evaporation of the solvent, the crude product (300 mg) was purified by Prep-HPLC with the following conditions (UV 254 nm/220 nm Welch Ultimate XB-C18 50*250, 10 μm Water (10 nmol/L NH₄HCO₃) ACN 60 mL/min 0% B to 3% B in 20 min 8.5) to afford [2-(2-aminoethoxy) ethyl]diethylamine (55 mg, 22.3%) as a white solid. MS: m/z: Calc'd for C₈H₂₀N₂O [M+H]⁺161. Found, 161.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1 eq.) and [2-(2-aminoethoxy)ethyl]diethylamine (54.5 mg, 0.34 mmol, 2 eq.) in ACN (10 mL) was added DIEA (65.9 mg, 0.51 mmol, 3 eq.) dropwise at room temperature under nitrogen atmosphere. The resulting mixture was stirred at room temperature for 6 h under nitrogen atmosphere. After completion of reaction monitored by LCMS. The residue was purified by reversed-phase flash chromatography with the following conditions: column, C18 silica gel; mobile phase, ACN in Water (0.5% TFA), 10% to 50% gradient in 20 min; detector, UV 254 nm. This resulted in tert-butyl (2R,3S,4S)-4-[(tert-butoxycarbonyl)oxy]-3-[({2-[2-(diethylamino)ethoxy]ethyl}carbamoyl)oxy]-2-[(4-methoxyphenyl)methyl]pyrrolidine-1-carboxylate (95 mg, 91.7%) as a colorless solid. MS: m/z: Calc'd for: C₃₁H₅₁N₃O₉ [M+H]⁺610. Found, 610.

Step 4: To a stirred solution of tert-butyl (2R,3S,4S)-4-[(tert-butoxycarbonyl)oxy]-3-[({2-[2-(diethylamino)ethoxy]ethyl}carbamoyl)oxy]-2-[(4-methoxyphenyl)methyl]pyrrolidine-1-carboxylate (90 mg, 0.15 mmol) in DCM (5 mL) was added TFA (1 mL) dropwise at room temperature under nitrogen atmosphere. The resulting mixture was stirred at room temperature for 2 h. After completion of reaction monitored by LCMS. The crude product (75 mg) was purified by Prep-HPLC to afford (2R,3S,4S)-4-hydroxy-2-[(4-methoxyphenyl)methyl]pyrrolidin-3-yl N-{2-[2-(diethylamino)ethoxy]ethyl}carbamate; trifluoroacetic acid (15.6 mg, 20.11%) as a light yellow semi-solid. MS: m/z: Calc'd for C₂₁H₃₂N₄O₄ [M+H]⁺410. Found, 410. ¹H NMR (400 MHz, Methanol-d₄) δ 7.28-7.21 (m, 2H), 6.98-6.90 (m, 2H), 4.98 (d, J=3.5 Hz, 1H), 4.41-4.35 (m, 1H), 4.22-4.13 (m, 1H), 3.90-3.82 (m, 2H), 3.80 (s, 3H), 3.64 (t, J=5.6 Hz, 2H), 3.58 (dd, J=12.7, 4.3 Hz, 1H), 3.45-3.36 (m, 4H), 3.31-3.15 (m, 5H), 3.11 (dd, J=14.2, 7.0 Hz, 1H), 2.98 (dd, J=14.1, 8.6 Hz, 1H), 1.33 (t, J=7.3 Hz, 6H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 19% B in 10 min; Wave Length: 254 nm/220 nm; RT1(min): 8.68.

### Synthetic Procedure of V187626

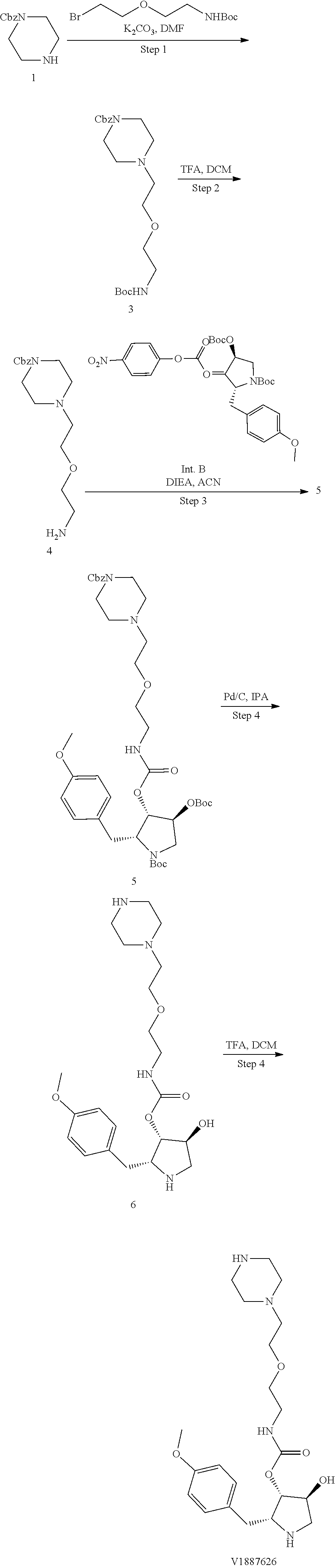

Step 1: To a stirred solution of benzyl piperazine-1-carboxylate (300 mg, 1.36 mmol, 1.0 eq.) and tert-butyl N-[2-(2-bromoethoxy)ethyl]carbamate (730.4 mg, 2.72 mmol, 2.0 eq.) in DMA (5 mL) was added K₂CO₃ (564.7 mg, 4.09 mmol, 3.0 eq.) in portions at room temperature. The resulting reaction mixture was stirred at ambient temperature for overnight. The resulting mixture was filtered, the filter cake was washed with DMA (1×1 mL). The residue was purified by reversed-phase flash chromatography with the following conditions: column, C18 silica gel; mobile phase, MeCN in Water (0.1% TFA), 10% to 50% gradient in 10 min; detector, UV 220 nm. This resulted in benzyl 4-(2-{2-[(tert-butoxycarbonyl)amino]ethoxy}ethyl)piperazine-1-carboxylate (500 mg, 90.1%) as a light yellow oil. MS: m/z: Calc'd for C₂₁H₃₃N₃O₅ [M+H]⁺408. Found, 408.

Step 2: To a stirred solution of benzyl 4-(2-{2-[(tert-butoxycarbonyl)amino]ethoxy}ethyl) piperazine-1-carboxylate (200 mg, 0.49 mmol, 1 eq.) in DCM (3 mL) was added TFA (1.5 mL) at room temperature. The resulting reaction mixture was stirred at ambient temperature for 1 h. The resulting mixture was concentrated under reduced pressure to afford benzyl 4-[2-(2-aminoethoxy)ethyl]piperazine-1-carboxylate (150 mg, 99.4%) as a light yellow oil. MS: m/z: Calc'd for C₁₆H₂₅N₃O₃ [M+H]⁺308. Found, 308.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.14 mmol, 1.0 eq.) and benzyl 4-[2-(2-aminoethoxy)ethyl]piperazine-1-carboxylate (83.6 mg, 0.27 mmol, 2.0 eq.) in MeCN (3 mL) was added DIEA (52.7 mg, 0.41 mmol, 3.0 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford benzyl 4-(2-{2-[({[(2R,3S,4S)-1-(tert-butoxycarbonyl)-4-[(tert-butoxycarbonyl)oxy]-2-[(4-methoxyphenyl) methyl]pyrrolidin-3-yl]oxy}carbonyl)amino]ethoxy}ethyl)piperazine-1-carboxylate (100 mg, 97.2%) as a light yellow oil. MS: m/z: Calc'd for C₃₉H₅₆N₄O₁₁ [M+H]⁺757. Found, 757.

Step 4: Under a nitrogen atmosphere, Pd/C (50 mg) was added to a solution of benzyl 4-(2-{2-[({[(2R,3S,4S)-1-(tert-butoxycarbonyl)-4-[(tert-butoxycarbonyl)oxy]-2-[(4-methoxyphenyl) methyl]pyrrolidin-3-yl]oxy}carbonyl)amino]ethoxy}ethyl)piperazine-1-carboxylate (100 mg, 0.13 mmol, 1.0 eq.) in IPA (3 mL), H2 was subsequently introduced into the reaction system, and the resulting mixture was stirred at r.t. for 2 h. Upon completion, the reaction mixture was filtered and concentrated in vacuo to afford crude product tert-butyl (2R,3S,4S)-4-[(tert-butoxycarbonyl)oxy]-2-[(4-methoxyphenyl)methyl]-3-[({2-[2-(piperazin-1-yl)ethoxy]ethyl}carbamoyl)oxy]pyrrolidine-1-carboxylate (80 mg, 97.2%) as a light yellow oil. MS: m/z: Calc'd for C₃₁H₅₀N₄O₉[M+H]⁺623. Found, 623.

Step 5: To a stirred solution of tert-butyl (2R,3S,4S)-4-[(tert-butoxycarbonyl)oxy]-2-[(4-methoxyphenyl)methyl]-3-[({2-[2-(piperazin-1-yl)ethoxy]ethyl}carbamoyl)oxy]pyrrolidine-1-carboxylate (95 mg, 0.15 mmol, 1 eq.) in DCM (3 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-{2-[2-(piperazin-1-yl)ethoxy]ethyl}carbamate; trifluoroacetic acid (21.4 mg, 25.9%) as a light yellow semi-solid. MS: m/z: Calc'd for C₂₁H₃₄N₄O₅ [M+H]⁺423. Found, 423. ¹H NMR (400 MHz, Methanol-d₄) δ 7.28-7.20 (m, 2H), 6.98-6.90 (m, 2H), 4.96 (d, J=3.5 Hz, 1H), 4.38 (d, J=4.2 Hz, 1H), 4.14-4.14 (m, 1H), 3.64-3.55 (m, 3H), 3.52-3.44 (m, 4H), 3.37 (dd, J=12.2, 5.7 Hz, 11H), 3.23-3.19 (m, 3H), 3.12 (dd, J=14.1, 7.0 Hz, 1H), 2.98 (dd, J=14.1, 8.6 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 15% B in 10 min; Wave Length: 254 nm/220 nm; RT1(min): 8.68

### Synthetic Procedure of V187627

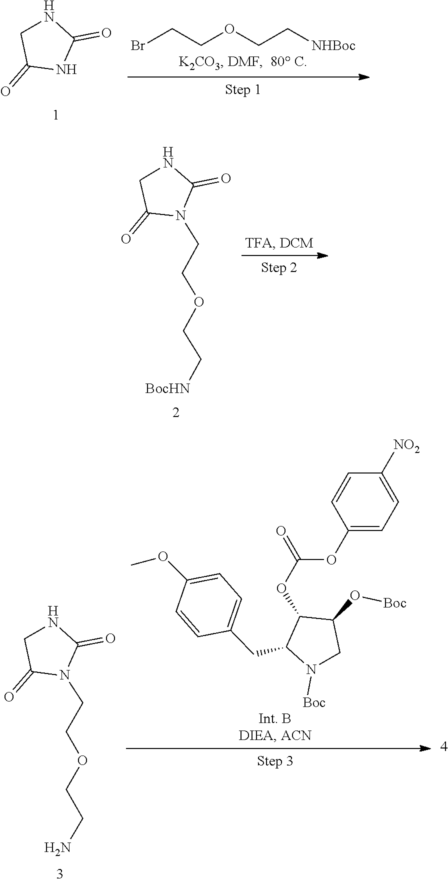

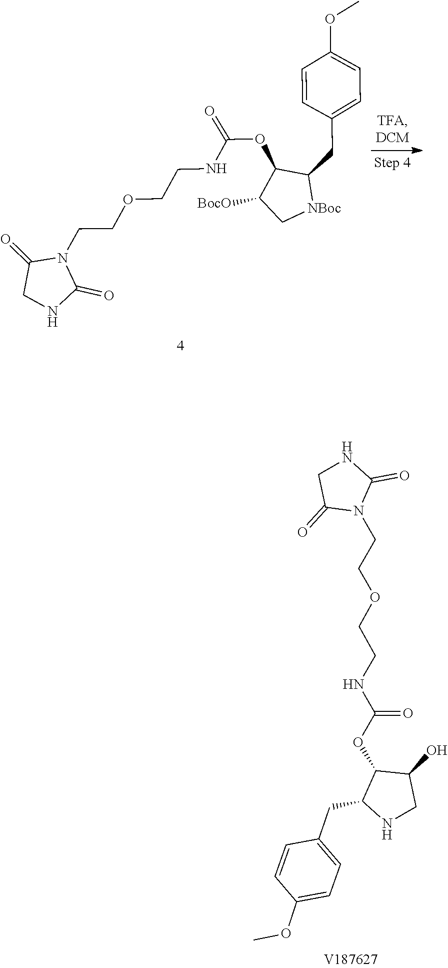

Step 1: To a stirred solution of hydantoin (300 mg, 3.00 mmol, 1 eq.) and K₂CO₃ (1243 mg, 8.99 mmol, 3 eq.) in DMF (4 mL) was added tert-butyl N-[2-(2-bromoethoxy)ethyl]carbamate (723 mg, 2.70 mmol, 0.9 eq.) at room temperature. The resulting mixture was stirred for 5 h at 80° C. After completion of the reaction monitored by LCMS. The resulting mixture was filtered, the filter cake was washed with MeCN (3×10 mL). The filtrate was concentrated under reduced pressure. The residue was purified by reversed-phase column chromatography (10 mmol/L NH₄HCO₃ in water and acetonitrile) to afford tert-butyl N-{2-[2-(2,5-dioxoimidazolidin-1-yl)ethoxy]ethyl}carbamate (92 mg, 10.7%) as a yellow oil. MS: m/z: Calc'd for C₁₂H₂₁N₃O₅ [M+H−100]⁺188. Found, 188.

Step 2: To a stirred solution of tert-butyl N-{2-[2-(2,5-dioxoimidazolidin-1-yl)ethoxy]ethyl}carbamate (92 mg, 0.32 mmol) in DCM (4 mL) was added TFA (2 mL) dropwise at room temperature. The resulting reaction mixture was stirred at room temperature for 1 h. After completion of the reaction monitored by LCMS, The resulting mixture was concentrated under reduced pressure to afford 3-[2-(2-aminoethoxy)ethyl]imidazolidine-2,4-dione (60 mg, 100%) as a yellow oil. MS: m/z: Calc'd for C₇H₁₃N₃O₃ [M+H]⁺188. Found, 188.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.14 mmol, 1 eq.) and 3-[2-(2-aminoethoxy)ethyl]imidazolidine-2,4-dione (51 mg, 0.27 mmol, 2 eq.) in ACN (4 mL) were added DIEA (53 mg, 0.41 mmol, 3 eq.). The resulting mixture was stirred overnight at room temperature. After completion of the reaction monitored by LCMS, the resulting mixture was purified directly by reversed-phase column chromatography (10 mmol/L NH₄HCO₃ in water and acetonitrile) to afford tert-butyl (2R,3S,4S)-4-[(tert-butoxycarbonyl)oxy]-3-[({2-[2-(2,5-dioxoimidazolidin-1-yl)ethoxy]ethyl}carbamoyl) oxy]-2-[(4-methoxyphenyl) methyl]pyrrolidine-1-carboxylate (76 mg, 87.8%) as a colorless oil. MS: m/z: Calc'd for C₃₀H₄₄N₄O₁₁ [M+H−100]⁺537. Found, 537.

Step 4: To a stirred solution of tert-butyl (2R,3S,4S)-4-[(tert-butoxycarbonyl)oxy]-3-[({2-[2-(2,5-dioxoimidazolidin-1-yl) ethoxy]ethyl}carbamoyl)oxy]-2-[(4-methoxyphenyl) methyl]pyrrolidine-1-carboxylate (76 mg, 0.12 mmol) in DCM (4 mL) was added TFA (4 mL) dropwise at room temperature. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl N-{2-[2-(2,5-dioxoimidazolidin-1-yl) ethoxy]ethyl}carbamate; trifluoroacetic acid (17.9 mg, 26.67%) as a white solid. MS: m/z: Calc'd for C₂₀H₂₈N₄O₇ [M+H]⁺437. Found, 437. ¹H NMR (400 MHz, Methanol-d4) δ 7.28-7.20 (m, 2H), 6.97-6.89 (m, 2H), 5.00-4.91 (m, 1H), 4.41 (d, J=4.3 Hz, 1H), 4.15-4.13 (m, 1H), 3.96 (d, J=2.2 Hz, 2H), 3.80 (d, J=2.0 Hz, 3H), 3.70-3.68 (m, 4H), 3.60-3.54 (m, 3H), 3.33 (d, J=10.7 Hz, 1H), 3.22 (d, J=12.6 Hz, 1H), 3.13 (dd, J=14.0, 7.4 Hz, 1H), 2.99 (dd, J=14.0, 8.3 Hz, 1H). Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 24% B in 10 min; Wave Length: 254 nm/220 nm; RT1(min): 8.68

### Synthetic Procedure of V187628

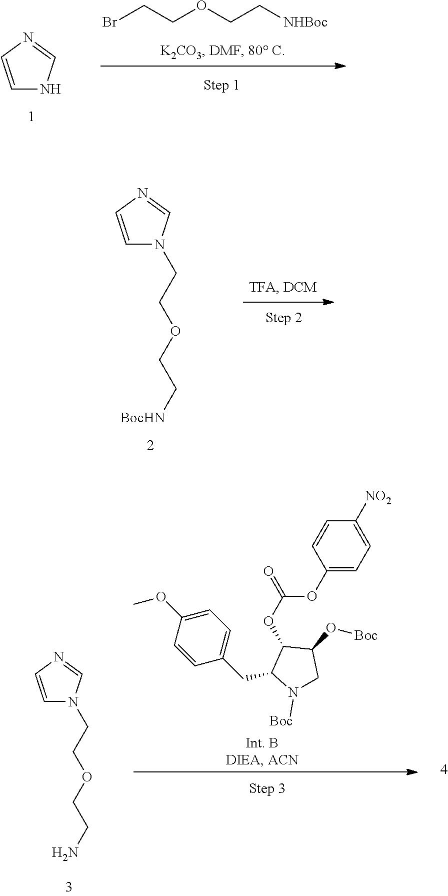 [SMILES: CC(C)(C)OC(=O)NCCOCCBr ; c1c[nH]cn1 ; CC(C)(C)OC(=O)NCCOCCn1ccnc1 ; NCCOCCn1ccnc1 ; COc1ccc(C[C@@H]2[C@H](OC(=O)Oc3ccc([N+](=O)[O-])cc3)[C@@H](OC(=O)OC(C)(C)C)CN2C(=O)OC(C)(C)C)cc1]

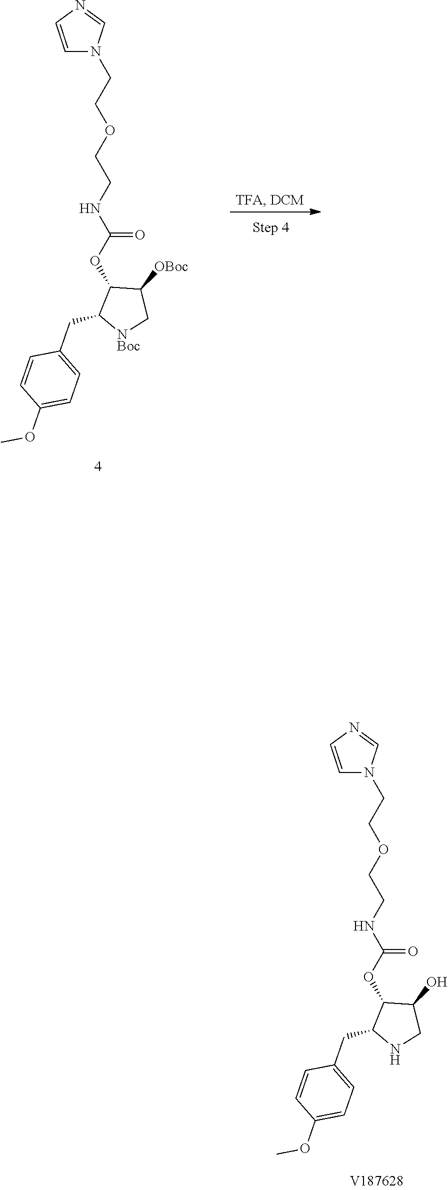

Step 1: To a stirred solution of imidazole (200 mg, 2.94 mmol, 1 eq.) and K₂CO₃ (1218 mg, 8.81 mmol, 3 eq.) in DMF (3 mL) was added tert-butyl N-[2-(2-bromoethoxy)ethyl]carbamate (788 mg, 2.94 mmol, 1 eq.) at room temperature. The resulting mixture was stirred at 80° C. for overnight. After completion of the reaction monitored by LCMS, the resulting mixture was filtered, the filter cake was washed with MeCN (3×10 mL). The filtrate was concentrated under reduced pressure. The residue was purified by reversed-phase column chromatography (10 mmol/L NH₄HCO₃ in water and acetonitrile) to afford tert-butyl N-{2-[2-(imidazol-1-yl)ethoxy]ethyl}carbamate (390 mg, 52.0%) as a yellow oil. MS: m/z: Calc'd for C₁₂H₂₁N₃O₃ [M+H]⁺256. Found, 256.

Step 2: To a stirred solution of tert-butyl N-{2-[2-(imidazol-1-yl)ethoxy]ethyl}carbamate (358 mg, 1.40 mmol) in DCM (8 mL) was added TFA (4 mL) dropwise at room temperature. The resulting reaction mixture was stirred at room temperature for 1 h. After completion of the reaction monitored by LCMS, The resulting mixture was concentrated under reduced pressure to afford 2-[2-(imidazol-1-yl)ethoxy]ethanamine (217 mg, 99.7%). MS: m/z: Calc'd for C₇H₁₃N₃O [M+H]⁺156. Found, 156.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.14 mmol, 1 eq.) and tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (80 mg, 0.14 mmol, 1 eq.) in ACN (4 mL) were added DIEA (53 mg, 0.41 mmol, 3 eq.). The resulting mixture was stirred overnight at room temperature. After completion of the reaction monitored by LCMS, the resulting mixture was purified directly by reversed-phase column chromatography (10 mmol/L NH₄HCO₃ in water and acetonitrile) to afford tert-butyl (2R,3S,4S)-4-[(tert-butoxycarbonyl) oxy]-3-[({2-[2-(imidazol-1-yl)ethoxy]ethyl}carbamoyl)oxy]-2-[(4-methoxyphenyl)methyl]pyrrolidine-1-carboxylate (82 mg, 99.8%) as a colorless oil. MS: m/z: Calc'd for C₃₀H₄₄N₄O₉[M+H]⁺605. Found, 605.

Step 4: To a stirred solution of tert-butyl (2R,3S,4S)-4-[(tert-butoxycarbonyl)oxy]-3-[({2-[2-(imidazol-1-yl) ethoxy]ethyl}carbamoyl) oxy]-2-[(4-methoxyphenyl) methyl]pyrrolidine-1-carboxylate (82 mg, 0.136 mmol) in DCM (4 mL) was added TFA (4 mL) at room temperature. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl N-{2-[2-(imidazol-1-yl)ethoxy]ethyl}carbamate; trifluoroacetic acid (16.3 mg, 23.0%) as a white solid. MS: m/z: Calc'd for C₂₀H₂₈N₄O₅ [M+H]⁺405. Found, 405. ¹H NMR (400 MHz, Methanol-d₄) δ 9.00-8.92 (m, 1H), 7.72-7.65 (m, 1H), 7.60-7.52 (m, 1H), 7.28-7.19 (m, 2H), 6.96-6.87 (m, 2H), 4.46 (s, 3H), 4.36 (s, 1H), 4.16 (s, 2H), 3.87 (s, 3H), 3.83 (d, J=11.4 Hz, 1H), 3.78 (d, J=7.2 Hz, 1H), 3.60 (s, 3H), 3.31-3.19 (m, 2H), 3.18-2.82 (m, 2H). Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 17% B in 10 min; Wave Length: 254 nm/220 nm; RT1(min): 8.68

### Synthetic Procedure of V187636

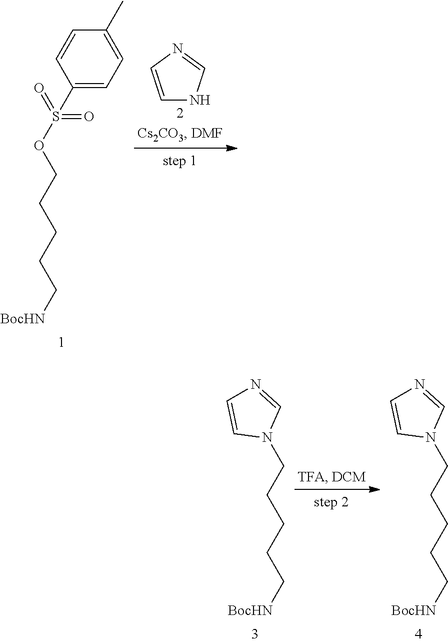

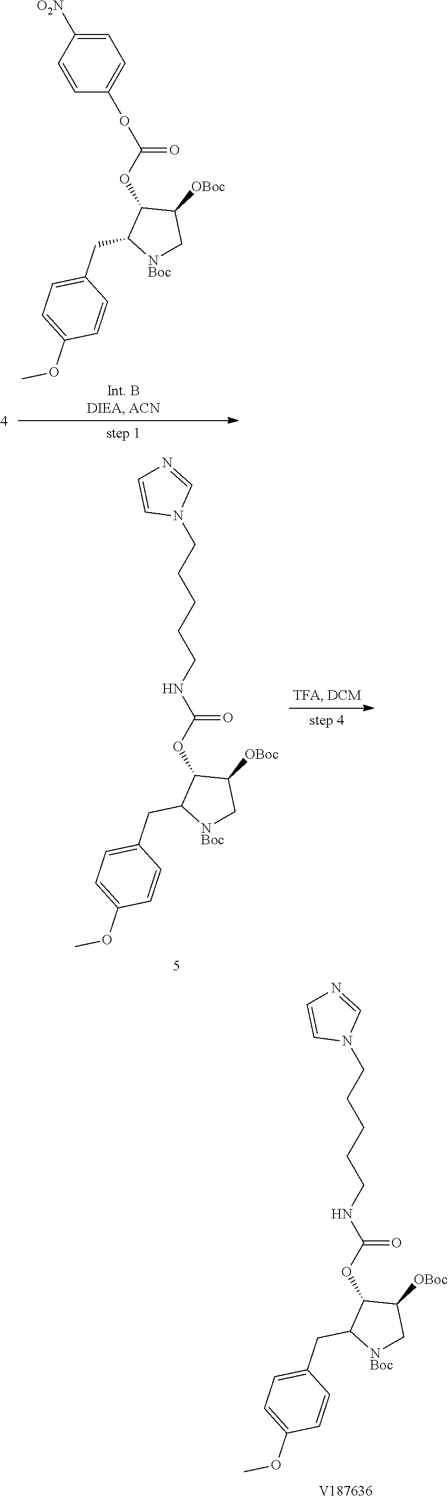

Step 1: To a stirred solution of tert-butyl N-{5-[(4-methylbenzenesulfonyl)oxy]pentyl}carbamate (300 mg, 0.84 mmol, 1.0 eq.) and imidazole (85.7 mg, 1.26 mmol, 1.5 eq.) in DMF (5 mL) was added Cs₂CO₃ (820.31 mg, 2.52 mmol, 3.0 eq.) at room temperature. The resulting reaction mixture was stirred at ambient temperature for overnight. The resulting mixture was filtered, the filter cake was washed with DMF (1×1 mL). The residue was purified by reversed-phase flash chromatography with the following conditions: column, C18 silica gel; mobile phase, MeCN in Water (0.1% TFA), 10% to 50% gradient in 10 min; detector, UV 220 nm. This resulted in tert-butyl N-[5-(imidazol-1-yl)pentyl]carbamate (200 mg, 94.1%) as a light yellow oil. MS: m/z: Calc'd for C₁₃H₂₃N₃O₂ [M+H]⁺254. Found, 254.

Step 2: To a stirred solution of tert-butyl N-[5-(imidazol-1-yl)pentyl]carbamate (100 mg, 0.40 mmol, 1.0 eq.) in DCM (2 mL) was added TFA (1 mL) at room temperature. The resulting reaction mixture was stirred at ambient temperature for 1 h. Desired product could be detected by LCMS. The resulting mixture was concentrated under reduced pressure. This resulted in 5-(imidazol-1-yl)pentan-1-amine (60 mg, 99.2%) as a light yellow oil. MS: m/z: Calc'd for C₈H₁₅N₃[M+H]⁺154. Found, 154.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.14 mmol, 1.0 eq.) and 5-(imidazol-1-yl)pentan-1-amine (41.7 mg, 0.27 mmol, 2.0 eq.) in MeCN (3 mL) was added DIEA (52.7 mg, 0.41 mmol, 3.0 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl) oxy]-3-({[5-(imidazol-1-yl)pentyl]carbamoyl}oxy)-2-[(4-methoxyphenyl)methyl]pyrrolidine-1-carboxylate (80 mg, 97.7%) as a light yellow oil. MS: m/z: Calc'd for C₃₁H₄₆N₄O₈ [M+H]⁺603. Found, 603.

Step 4: To a stirred solution of tert-butyl (2R,3S,4S)-4-[(tert-butoxycarbonyl)oxy]-3-({[5-(imidazol-1-yl)pentyl]carbamoyl}oxy)-2-[(4-methoxyphenyl)methyl]pyrrolidine-1-carboxylate (75 mg, 0.12 mmol, 1.0 eq.) in DCM (3 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-[5-(imidazol-1-yl)pentyl]carbamate; trifluoroacetic acid (15.7 mg, 24.2%) as a light yellow semi-solid. MS: m/z: Calc'd for C₂₁H₃₀N₄O₄[M+H]⁺423. Found, 423. ¹H NMR (400 MHz, Methanol-d₄) δ 8.96 (s, 1H), 7.67 (s, 1H), 7.57 (s, 1H), 7.28-7.18 (m, 2H), 6.92 (d, J=8.4 Hz, 2H), 4.93 (d, J=3.4 Hz, 1H), 4.36 (d, J=4.2 Hz, 1H), 4.30-4.26 (m, 2H), 4.17-4.12 (m, 1H), 3.79 (s, 3H), 3.60-3.55 (m, 1H), 3.26-3.04 (m, 4H), 3.00-2.94 (m, 1H), 2.00-1.93 (m, 2H), 1.66-1.58 (m, 2H), 1.45-1.37 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 21% B in 10 min; Wave Length: 254 nm/220 nm; RT1(min): 8.5

### Synthetic Procedure of V187637

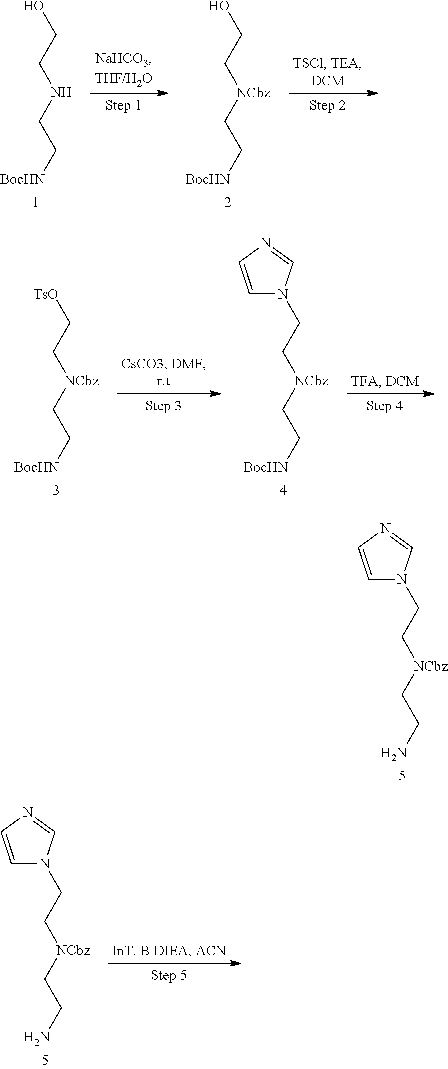 [SMILES: CC(C)(C)OC(=O)NCCNCCO ; CC(C)(C)OC(=O)NCCN(CCO)C(=O)OCc1ccccc1 ; Cc1ccc(S(=O)(=O)OCCN(CCNC(=O)OC(C)(C)C)C(=O)OCc2ccccc2)cc1 ; CC(C)(C)OC(=O)NCCN(CCn1ccnc1)C(=O)OCc1ccccc1 ; NCCN(CCn1ccnc1)C(=O)OCc1ccccc1 ; NCCN(CCn1ccnc1)C(=O)OCc1ccccc1]

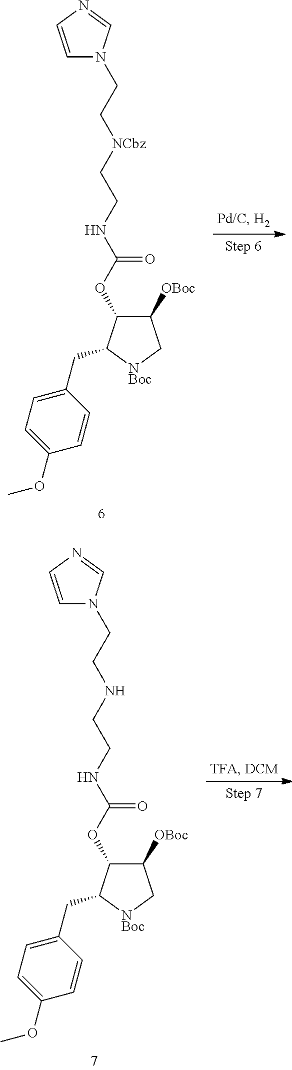

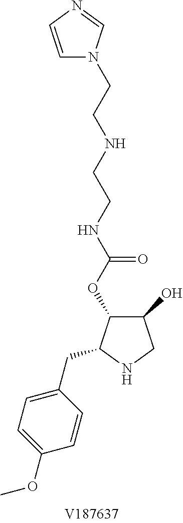 [SMILES: COc1ccc(C[C@H]2NC[C@H](O)[C@H]2OC(=O)NCCNCCn2ccnc2)cc1]

Step 1: To the solution of tert-butyl N-{2-[(2-hydroxyethyl)amino]ethyl}carbamate (500 mg, 2.45 mmol, 1 equiv) and CbzCl (543 mg, 3.18 mmol, 1.3 equiv) in THF (10 mL) and H₂O (2 mL) was added NaHCO₃ (617 mg, 7.34 mmol, 3 equiv). The mixture was stirred at room temperature for overnight. LCMS showed the reaction was completed. The mixture was diluted with EA, washed with water, dried and concentrated. The resulted crude product was purified by Prep-TLC. The tert-butyl N-(2-{[(benzyloxy)carbonyl](2-hydroxyethyl)amino}ethyl)carbamate (646 mg, 78.0%) was obtained as a colorless oil. MS: m/z: Calc'd for C₁₇H₂₆N₂O₅ [M+H]⁺339. found 339.

Step 2: To the solution of tert-butyl N-(2-{[(benzyloxy)carbonyl](2-hydroxyethyl)amino}ethyl) carbamate (430 mg, 1.27 mmol, 1 equiv) and DMAP (16 mg, 0.12 mmol, 0.1 equiv) in DCM (10 mL) was added TEA (193 mg, 1.90 mmol, 1.5 equiv) and TsCl (315 mg, 1.65 mmol, 1.3 equiv) slowly. The mixture was stirred at room temperature for 5 hours. LCMS showed the reaction was OK. The resulting mixture was concentrated under reduced pressure. The residue was purified by Prep-TLC (PE/EA 2:1) to afford tert-butyl N-(2-{[(benzyloxy)carbonyl]({2-[(4-methyl benzenesulfonyl)oxy]ethyl})amino}ethyl)carbamate (300 mg, 47.9%) as a colorless oil. MS: m/z: Calc'd for C₂₄H₃₂N₂O₇S [M+H−100]⁺393. found 393.

Step 3: To a stirred mixture of tert-butyl N-(2-{[(benzyloxy)carbonyl]({2-[(4-methylbenzene sulfonyl)oxy]ethyl})amino}ethyl)carbamate (220 mg, 0.44 mmol, 1 equiv) and imidazole (61 mg, 0.89 mmol, 2 equiv) in DMF (5 mL) was added Cs₂CO₃ (436 mg, 1.34 mmol, 3 equiv) at 0° C. The resulting mixture was stirred for overnight at room temperature. Desired product could be detected by LCMS. The mixture was filtered and the crude product was purified by reversed-phase flash chromatography with the following conditions: column, C18 silica gel; mobile phase, MeCN in Water (0.1% TFA), 50% to 60% gradient in 10 min; detector, UV 254 nm. to afford tert-butyl N-(2-{[(benzyloxy)carbonyl][2-(imidazol-1-yl)ethyl]amino}ethyl)carbamate (120 mg, 69.1%) as a yellow solid. MS: m/z: Calc'd for C₂₀H₂₈N₄O₄ [M+H]⁺389. found 389.

Step 4: To the solution of tert-butyl N-(2-{[(benzyloxy)carbonyl][2-(imidazol-1-yl)ethyl]amino}ethyl)carbamate (220 mg, 0.57 mmol, 1 equiv) in DCM (6 mL) was added TFA (2 mL) dropwise. The mixture was stirred at room temperature for 2 hours. Desired product could be detected by LCMS. The mixture was concentrated under reduced pressure. The crude product was used in the next step directly without further purification. MS: m/z: Calc'd for C₁₅H₂₀N₄O₂ [M+H]⁺289. found 289.

Step 5: A solution of tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (100 mg, 0.17 mmol, 1 equiv), benzyl N-(2-aminoethyl)-N-[2-(imidazol-1-yl)ethyl]carbamate (49 mg, 0.17 mmol, 1 equiv) and DIEA (44 mg, 0.34 mmol, 2 equiv) in MeCN (5 ml) was stirred for 2 h at room temperature under nitrogen atmosphere. Desired product could be detected by LCMS. The mixture was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in tert-butyl (2R,3S,4S)-3-{[(2-{[(benzyloxy)carbonyl][2-(imidazol-1-yl)ethyl]amino}ethyl)carbamoyl]oxy}-4-[(tert-butoxycarbonyl)oxy]-2-[(4-methoxyphenyl)methyl]pyrrolidine-1-carboxylate (48 mg, 38.3%) as a yellow oil. MS: m/z: Calc'd for C₃₈H₅₁N₅O₁₀ [M+H]⁺738. Found, 738.

Step 6: Under a nitrogen atmosphere, Pd/C (45 mg) was added to a solution of tert-butyl (2R,3S,4S)-3-{[(2-{[(benzyloxy)carbonyl][2-(imidazol-1-yl)ethyl]amino}ethyl)carbamoyl]oxy}-4-[(tert-butoxycarbonyl)oxy]-2-[(4-methoxyphenyl)methyl]pyrrolidine-1-carboxylate (43 mg, 0.06 mmol, 1 equiv) in IPA (3 mL), H2 was subsequently introduced into the reaction system, and the resulting mixture was stirred at r.t. for 2 h. Upon completion, the reaction mixture was filtered and concentrated in vacuo to afford crude product was used in the next step directly without further Purification. MS: m/z: Calc'd for C₃₀H₄₅N₅O₈ [M+H]⁺604. Found, 604.

Step 7: To a solution of tert-butyl (2R,3S,4S)-4-[(tert-butoxycarbonyl)oxy]-3-{[(2-{[2-(imidazol-1-yl)ethyl]amino}ethyl)carbamoyl]oxy}-2-[(4-methoxyphenyl)methyl]pyrrolidine-1-carboxylate (30 mg, 0.05 mmol, 1 equiv) in DCM (3 ml) was added TFA (1 mL). The mixture was stirred at room temperature for 2 hours. Desired product could be detected by LCMS. The mixture was concentrated under reduced pressure. The crude product (30 mg) was purified by Prep-HPLC to afford (2R,3S,4S)-4-hydroxy-2-[(4-methoxyphenyl)methyl]pyrrolidin-3-yl N-(2-{[2-(imidazol-1-yl)ethyl]amino}ethyl)carbamate (20 mg, 98%) as a brown semi-solid. MS: m/z: Calc'd for C₂₀H₂₉N₅O₄ [M+H]⁺404. Found, 404. ¹H NMR (400 MHz, Methanol-d₄) δ 7.69 (s, 1H), 7.20-7.10 (m, 3H), 6.98 (s, 1H), 6.88-6.81 (m, 2H), 4.71 (s, 1H), 4.65 (t, J=2.7 Hz, 1H), 4.14-4.10 (m, 3H), 3.77 (d, J=2.0 Hz, 3H), 3.60-3.54 (m, 1H), 3.27-3.22 (m, 2H), 3.05-2.97 (m, 2H), 2.86-2.72 (m, 5H). Prep-HPLC-conditions: Column: XBridge Prep OBD C18 Column, 30*150 mm, 5 μm; Mobile Phase A: Water (10 nmol/L NH₄HCO₃), Mobile Phase B: ACN; Flow rate: 60 mL/min mL/min; Gradient: 2% B to 29% B in 13 min; Wave Length: 254 nm/220 nm; RT1(min): 8.55

### Synthetic Procedure of V187638

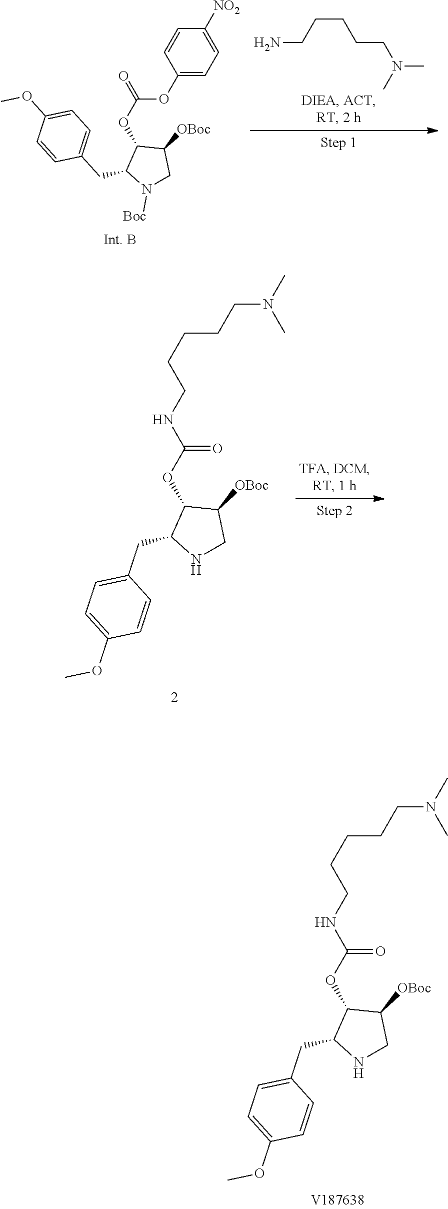

Step 1: To a stirred mixture of (5-aminopentyl)dimethylamine (44 mg, 0.34 mmol, 2 equiv) in ACN (5 mL) were added tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.00 equiv) and DIEA (88 mg, 0.68 mmol, 4 eq.). The resulting mixture was stirred for overnight at room temperature under air atmosphere. Desired product could be detected by LCMS. The mixture was concentrated under vacuum. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in tert-butyl (2R,3S,4S)-4-[(tert-butoxycarbonyl)oxy]-3-({[5-(dimethylamino)pentyl]carbamoyl}oxy)-2-[(4-methoxyphenyl)methyl]pyrrolidine-1-carboxylate (100 mg, 99.8%) as a yellow oil. MS: m/z: Calc'd for C₃₀H₄₉N₃O₈ [M+H]⁺580. found 580.

Step 2: To a stirred solution of tert-butyl (2R,3S,4S)-4-[(tert-butoxycarbonyl)oxy]-3-({[5-(dimethylamino)pentyl]carbamoyl}oxy)-2-[(4-methoxyphenyl)methyl]pyrrolidine-1-carboxylate (70 mg, 0.12 mmol, 1 equiv) in DCM (5 mL) was added TFA (1 mL) at 0° C. under air atmosphere. The resulting mixture was stirred for 2 h at room temperature. Desired product could be detected by LCMS. The mixture was concentrated under vacuum. The crude product (50 mg) was purified by Prep-HPLC to afford (2R,3S,4S)-4-hydroxy-2-[(4-methoxyphenyl) methyl]pyrrolidin-3-yl N-[5-(dimethylamino)pentyl]carbamate (25.7 mg, 42.8%) as a yellow oil. MS: m/z: Calc'd for C₂₀H₃₃N₃O₄ [M+H]⁺380. found 380. ¹H NMR (400 MHz, Methanol-d₄) δ 7.28-7.19 (m, 2H), 6.96-6.89 (m, 2H), 4.97-4.91 (m, 1H), 4.37 (d, J=4.2 Hz, 1H), 4.16-4.04 (m, 1H), 3.80 (s, 3H), 3.57 (dd, J=12.7, 4.3 Hz, 1H), 3.31-3.04 (m, 6H), 2.97 (dd, J=14.1, 8.5 Hz, 1H), 2.89 (s, 6H), 1.84-1.71 (m, 2H), 1.63-1.51 (m, 2H), 1.45-1.32 (m, 2H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 20% B in 10 min; Wave Length: 254 nm/220 nm; RT1(min): 8.68

### Synthetic Procedure of V187640

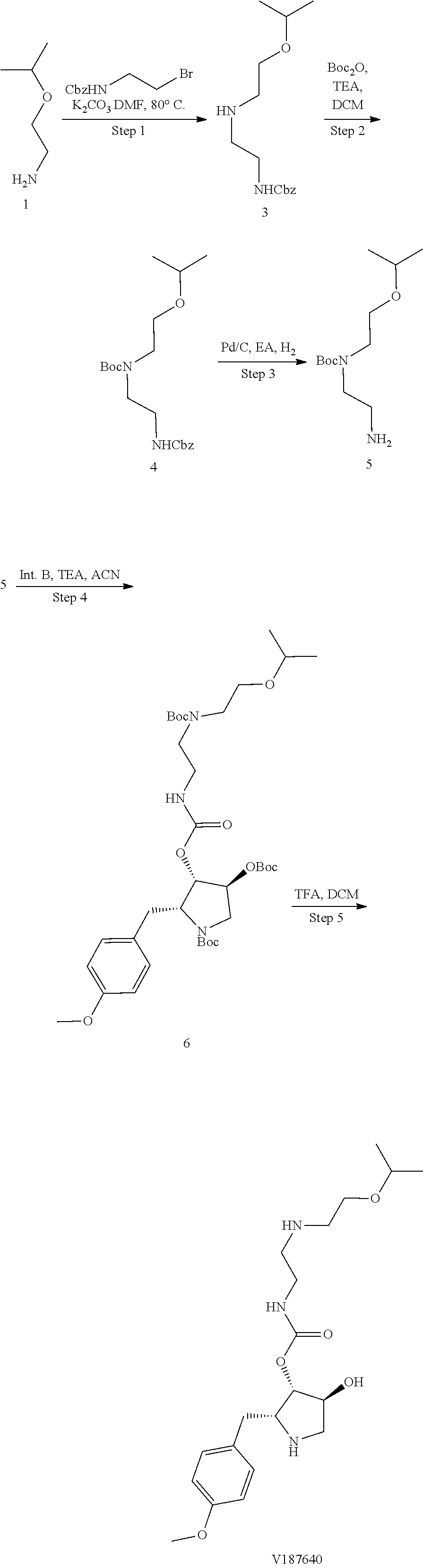

Step 1: To the mixture of 2-isopropoxyethanamine (1 g, 19.4 mmol, 10 eq.) and benzyl N-(2-bromoethyl)carbamate (500 mg, 1.94 mmol, 1 eq.) in DMF (1 mL) was added K₂CO₃ (803 mg, 5.81 mmol, 2 eq.). The mixture was stirred at 80° C. for 2 hours. LCMS showed the reaction was OK. The mixture was diluted with EA, washed with water, dried and concentrated to give the crude product which was used in the next step directly without further purification. MS: m/z: Calc'd for C₁₅H₂₄N₂O₃ [M+H]⁺281. found 281.

Step 2: To the solution of benzyl N-{2-[(2-isopropoxyethyl)amino]ethyl}carbamate (470 mg, 1.68 mmol, 1 eq.) in DCM (10 mL) were added TEA (339 mg, 3.35 mmol, 2 eq.) and Boc₂O (549 mg, 2.51 mmol, 1.5 eq.). The mixture was stirred at rt for 1 hour. Upon completion, the mixture was diluted with DCM, washed with water, dried and concentrated. The residue was purified by reversed phase column chromatography to afford benzyl N-{2-[(tert-butoxycarbonyl) (2-isopropoxyethyl)amino]ethyl}carbamate (510 mg, 89.0%) as a yellow oil. MS: m/z: Calc'd for C₂₀H₃₂N₂O₅ [M+H]⁺381. found 381.

Step 3: Under a nitrogen atmosphere, Pd/C (100 mg) was added to a solution of benzyl N-{2-[(tert-butoxycarbonyl)(2-isopropoxyethyl)amino]ethyl}carbamate (210 mg, 0.55 mmol) in EA (5 mL). H₂ was subsequently introduced into the reaction system, and the resulting mixture was stirred at r.t. for overnight. Upon completion, the reaction mixture was filtered through a Celite pad and concentrated in vacuo to give the crude product which was used directly in the next step without further purification. MS: m/z: Calc'd for C₁₂H₂₆N₂O₃ [M+H]⁺247. found 247.

Step 4: To the solution of tert-butyl (2R,3S,4S)-4-[(tert-butoxycarbonyl)oxy]-2-[(4-methoxy phenyl) methyl]-3-[(4-nitrophenoxycarbonyl)oxy]pyrrolidine-1-carboxylate (80 mg, 0.14 mmol, 1 eq.) and tert-butyl N-(2-aminoethyl)-N-(2-isopropoxyethyl)carbamate (40 mg, 0.16 mmol, 1.14 eq.) in ACN (5 mL) was added DIEA (35 mg, 0.27 mmol, 2 eq.). The mixture was stirred at room temperature for 1 hour. Upon completion, the reaction mixture was concentrated in vacuo. The residue was purified by reversed phase column chromatography to afford tert-butyl (2R,3S,4S)-3-[({2-[(tert-butoxycarbonyl)(2-isopropoxyethyl)amino]ethyl}carbamoyl)oxy]-4-[(tert-butoxycarbonyl)oxy]-2-[(4-methoxyphenyl)methyl]pyrrolidine-1-carboxylate (88 mg, 93.0%) was obtained as a colorless oil. MS: m/z: Calc'd for C₃₅H₅₇N₃O₁₁ [M+H−100]⁺596. found 596.

Step 5: To the solution of tert-butyl (2R,3S,4S)-3-[({2-[(tert-butoxycarbonyl)(2-isopropoxy ethyl)amino]ethyl}carbamoyl)oxy]-4-[(tert-butoxycarbonyl)oxy]-2-[(4-methoxyphenyl)methyl]pyrrolidine-1-carboxylate (80 mg, 0.12 mmol) in DCM (5 mL) was added TFA (1 mL) slowly. The mixture was stirred at rt for 1 hour. Upon completion, the reaction mixture was concentrated in vacuo. The resulting residue was purified by Prep HPLC. To afford (2R,3S,4S)-4-hydroxy-2-[(4-methoxyphenyl)methyl]pyrrolidin-3-yl N-{2-[(2-isopropoxyethyl) amino]ethyl}carbamate; trifluoroacetic acid (21.9 mg, 36.9%) as a colorless semi-solid. MS: m/z: Calc'd for C₂₀H₃₃N₃O₅ [M+H]⁺396. found 396. ¹H NMR (400 MHz, Methanol-d₄) δ 7.29-7.22 (m, 2H), 6.97-6.90 (m, 2H), 5.00 (d, J=3.8 Hz, 1H), 4.43 (d, J=4.9 Hz, 1H), 4.23-4.13 (m, 1H), 3.80 (s, 3H), 3.75-3.63 (m, 3H), 3.63-3.45 (m, 3H), 3.31-3.18 (m, 5H), 3.12 (dd, J=14.3, 6.7 Hz, 1H), 3.05-2.94 (m, 1H), 1.21 (d, J=6.2 Hz, 6H). Prep-HPLC-conditions: Column: Column: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 26% B in 10 min; Wave Length: 254 nm/220 nm; RT1(min): 8.68.

### Synthetic Procedure of V187641

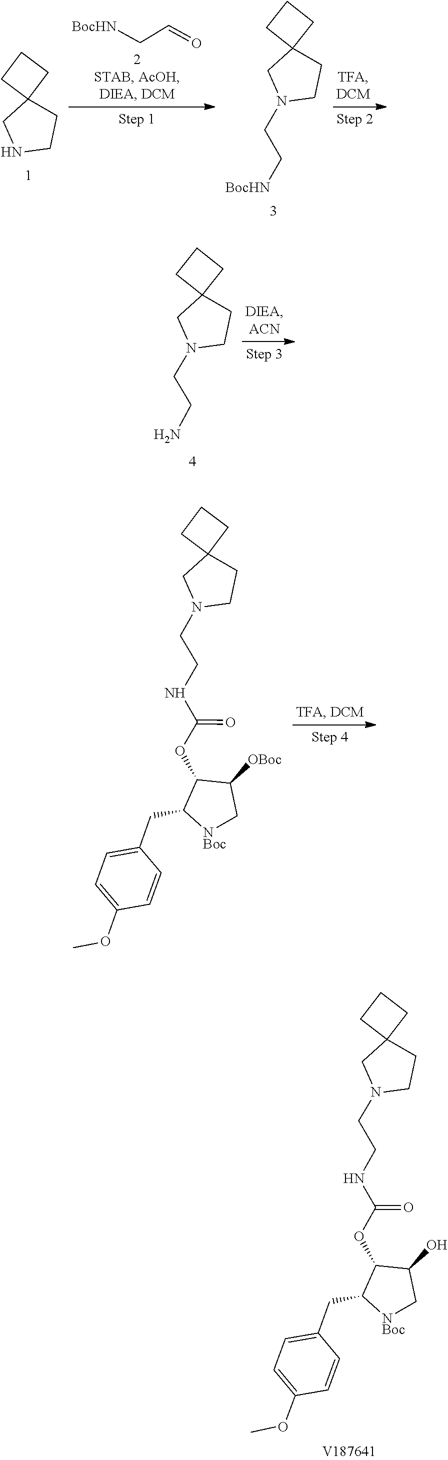

Step 1: A solution of 6-azaspiro[3.4]octane (150 mg, 1.35 mmol, 1 equiv) and HOAc (243 g, 4.05 mmol, 3 equiv) in DCM (5 mL) was treated with tert-butyl N-(2-oxoethyl)carbamate (279 mg, 1.75 mmol, 1.3 equiv) for 1 h at room temperature under nitrogen atmosphere followed by the addition of NaBH(OAc)₃ (572 mg, 2.69 mmol, 2 equiv). The resulting mixture was stirred for 2 h at room temperature under air atmosphere. Desired product could be detected by LCMS. The mixture was extracted with EtOAc (2×100 mL). The combined organic layers were washed with water (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in tert-butyl N-(2-{6-azaspiro[3.4]octan-6-yl}ethyl)carbamate (96 mg, 27.9%) as a white oil. MS: m/z: Calc'd for C₁₄H₂₆N₂O₂ [M+H]⁺255. found 255.

Step 2: To a stirred mixture of tert-butyl N-(2-{6-azaspiro[3.4]octan-6-yl}ethyl)carbamate (88 mg, 0.34 mmol, 1 equiv) in DCM (5 mL) was added TFA (1 mL) at 0° C. The resulting mixture was stirred for 1 h at room temperature. Desired product could be detected by LCMS. The resulting mixture was concentrated under reduced pressure. This resulted in 2-{6-azaspiro [3.4]octan-6-yl}ethanamine (60 mg, 92.4%) as a yellow oil. MS: m/z: Calc'd for C₉H₁₈N₂ [M+H]⁺155. found 155.

Step 3: To a stirred mixture of 2-{6-azaspiro[3.4]octan-6-yl}ethanamine (52 mg, 0.34 mmol, 2 equiv) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.00 equiv) in ACN (5 mL) was added DIEA (66 mg, 0.51 mmol, 3 equiv). The resulting mixture was stirred for overnight at room temperature. The resulting mixture was concentrated under vacuum. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in tert-butyl (2R,3S,4S)-3-{[(2-{6-azaspiro[3.4]octan-6-yl}ethyl)carbamoyl]oxy}-4-[(tert-butoxycarbonyl)oxy]-2-[(4-methoxyphenyl)methyl]pyrrolidine-1-carboxylate as a yellow solid. MS: m/z: Calc'd for C₃₂H₄₉N₃O₈ [M+H]⁺604. found 604.

Step 4: To a stirred solution of tert-butyl (2R,3S,4S)-3-{[(2-{6-azaspiro[3.4]octan-6-yl}ethyl) carbamoyl]oxy}-4-[(tert-butoxycarbonyl)oxy]-2-[(4-methoxyphenyl)methyl]pyrrolidine-1-carboxylate (50 mg, 0.08 mmol, 1 equiv) in DCM (5 mL) was added TFA (1 mL) at 0° C. The resulting mixture was stirred for 2 h at room temperature. Desired product could be detected by LCMS. The resulting mixture was concentrated under vacuum. The crude product (30 mg) was purified by Prep-HPLC to afford (2R,3S,4S)-4-hydroxy-2-[(4-methoxyphenyl) methyl]pyrrolidin-3-yl N-(2-{6-azaspiro[3.4]octan-6-yl}ethyl)carbamate (15.5 mg, 33.9%) as a brown solid. MS: m/z: Calc'd for C₂₂H₃₃N₃O₄ [M+H]⁺404. found 404. ¹H NMR (400 MHz, Methanol-d₄) δ 7.29-7.21 (m, 2H), 6.98-6.89 (m, 2H), 5.02 (d, J=4.0 Hz, 1H), 4.42 (d, J=4.2 Hz, 1H), 4.18-4.09 (m, 1H), 3.80 (s, 3H), 3.68-3.45 (m, 4H), 3.35 (d, J=6.1 Hz, 2H), 3.32 (s, 2H), 3.32-3.18 (m, 2H), 3.12 (dd, J=14.4, 6.6 Hz, 1H), 2.98 (dd, J=14.3, 9.0 Hz, 1H), 2.26-2.15 (m, 4H), 2.14-2.05 (m, 2H), 2.04-1.86 (m, 2H). Prep-HPLC-conditions: Column: Welch Ultimate XB-C18 50*250, 10 μm; Mobile Phase A: Water (0.05% TFA), Mobile Phase B: ACN; Flow rate: 60 mL/min mL/min; Gradient: 2% B to 50% B in 20 min; Wave Length: 254 nm/220 nm; RT1(min): 16

### Synthetic Procedure of V187643

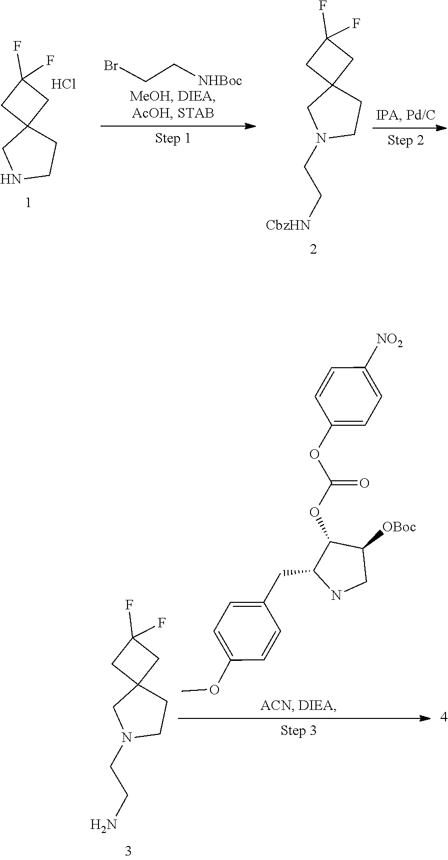

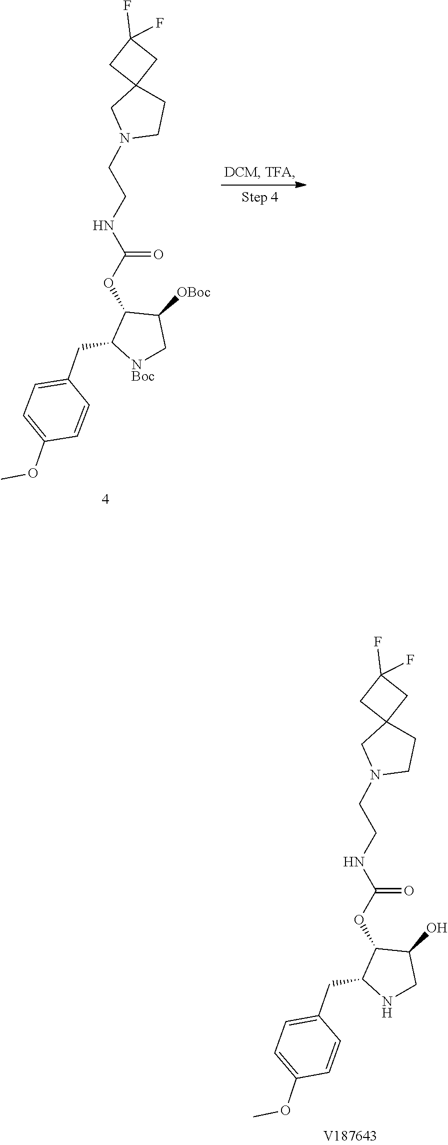

Step 1: To a stirred solution of 2,2-difluoro-6-azaspiro[3.4]octane hydrochloride (185 mg, 1.01 mmol, 1 eq.) and K₂CO₃ (418 mg, 3.02 mmol, 3 eq.) in ACN (8 mL) was added tert-butyl N-(2-bromoethyl)carbamate (271 mg, 1.21 mmol, 1.2 eq.) at room temperature. The resulting mixture was stirred overnight at 60° C. After completion of the reaction monitored by LCMS, the resulting mixture was filtered, the filter cake was washed with MeCN (3×10 mL). The filtrate was concentrated under reduced pressure. The residue was purified by reversed-phase flash chromatography with the following conditions: column, C18 silica gel; mobile phase, MeCN in Water (10 mmol/L NH₄HCO3), 0% to 60% gradient in 20 min; detector, UV 220 nm to afford benzyl N-(2-{2,2-difluoro-6-azaspiro[3.4]octan-6-yl}ethyl)carbamate (313 mg, 95.78%) as a light yellow oil. MS: m/z: Calc'd for C₁₇H₂₂F₂N₂O₂[M+H]⁺325. found 325.

Step 2: Under a nitrogen atmosphere, Pd/C (150 mg) was added to a solution of benzyl N-(2-{2,2-difluoro-6-azaspiro[3.4]octan-6-yl}ethyl)carbamate (150 mg, 0.46 mmol, 1 equiv) in IPA (5 mL), H2 was subsequently introduced into the reaction system, and the resulting mixture was stirred at r.t. for 2 h. Upon completion, the reaction mixture was filtered and concentrated in vacuo to afford crude product which was used in the next step directly without further purification. MS: m/z: Calc'd for C₉H₁₆F₂N₂ [M+H]⁺191. Found, 191.

Step 3: To a solution mixture of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (80 mg, 0.14 mmol, 1.00 equiv) and 2-{2,2-difluoro-6-azaspiro[3.4]octan-6-yl}ethanamine (31 mg, 0.16 mmol, 1.2 equiv) in ACN (3 mL) was added DIEA (0.2 mL, 0.27 mmol, 2 equiv) dropwise at room temperature. The resulting mixture was stirred at room temperature for 2 h. Desired product could be detected by LCMS. The residue was purified by reversed-phase flash chromatography with the following conditions: column, C18 silica gel; mobile phase, NH₄HCO₃ in ACN, 10% to 50% gradient in 10 min; detector, UV 254 nm. afford tert-butyl (2R,3S,4S)-4-[(tert-butoxycarbonyl)oxy]-3-{[(2-{2,2-difluoro-6-azaspiro[3.4]octan-6-yl}ethyl)carbamoyl]oxy}-2-[(4-methoxyphenyl)methyl]pyrrolidine-1-carboxylate (43 mg, 49.5%) as a white solid. MS: m/z: Calc'd for C₃₂H₄₇F₂N₃O₈ [M+H]⁺640. Found, 640.

Step 4: To a solution of tert-butyl (2R,3S,4S)-4-[(tert-butoxycarbonyl)oxy]-3-{[(2-{2,2-difluoro-6-azaspiro[3.4]octan-6-yl}ethyl)carbamoyl]oxy}-2-[(4-methoxyphenyl)methyl]pyrrolidine-1-carboxylate (43 mg, 0.07 mmol) in DCM (3 mL) was added TFA (1 mL) dropwise at room temperature. The resulting mixture was stirred for 2 h at room temperature. Upon completion, the reaction mixture was concentrated in vacuo. The crude product (30 mg) was purified by Prep-HPLC to afford (2R,3S,4S)-4-hydroxy-2-[(4-methoxyphenyl)methyl]pyrrolidin-3-yl N-(2-{2,2-difluoro-6-azaspiro[3.4]octan-6-yl}ethyl)carbamate (10.8 mg, 35.7%) as a yellow solid. MS: m/z: Calc'd for C₁₇H₂₂N₄O₄ [M+H]⁺440. Found, 440. ¹H NMR (400 MHz, Methanol-d₄) δ 7.14 (d, J=8.3 Hz, 2H), 6.85 (d, J=8.2 Hz, 2H), 4.64 (d, J=3.7 Hz, 1H), 4.16 (dd, J=6.1, 2.5 Hz, 1H), 3.78 (s, 3H), 3.52 (m, 1H), 3.39-3.25 (m, 4H), 2.83 (dd, J=13.5, 8.0 Hz, 1H), 2.78-2.69 (m, 5H), 2.59 (m, 6H), 2.03 (t, J=7.1 Hz, 2H). Prep-HPLC-conditions: Column: XBridge Prep OBD C18 Column, 30*150 mm, 5 μm; Mobile Phase A: Water (10nmol/L NH₄HCO₃), Mobile Phase B: ACN; Flow rate: 60 mL/min mL/min; Gradient: 16% B to 46% B in 13 min; Wave Length: 254 nm/220 nm; RT1(min): 8.28

### Synthetic Procedure of V187644

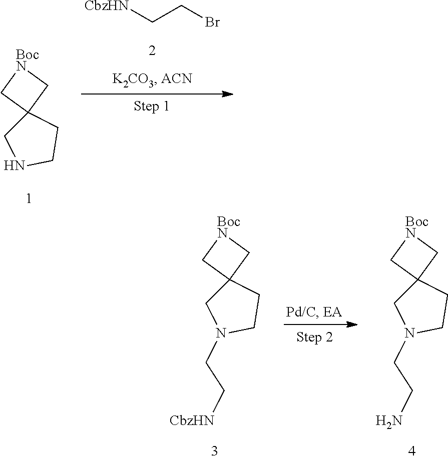 [SMILES: O=C(NCCBr)OCc1ccccc1 ; CC(C)(C)OC(=O)N1CC2(CCNC2)C1 ; CC(C)(C)OC(=O)N1CC2(CCN(CCNC(=O)OCc3ccccc3)C2)C1 ; CC(C)(C)OC(=O)N1CC2(CCN(CCN)C2)C1]

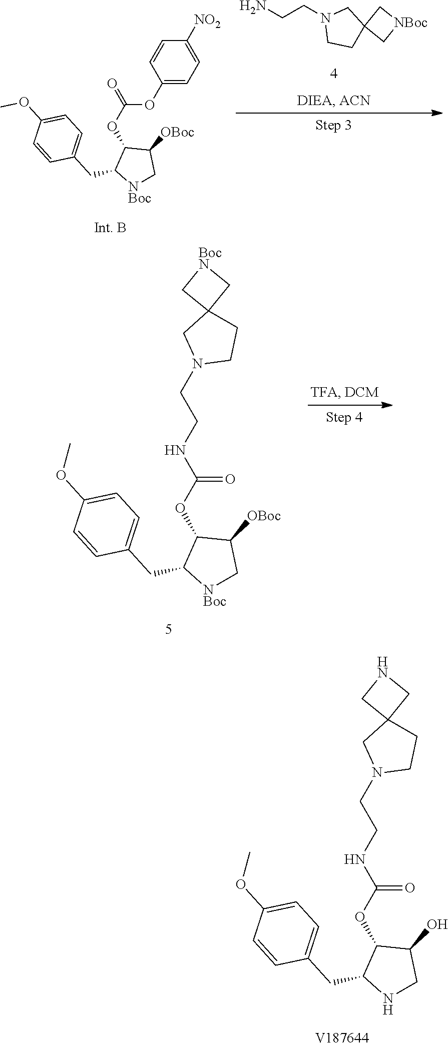

Step 1: To a stirred solution of tert-butyl 2,6-diazaspiro[3.4]octane-2-carboxylate (200 mg, 0.94 mmol, 1 eq.) and benzyl N-(2-bromoethyl)carbamate (243.17 mg, 0.942 mmol, 1 eq.) in ACN (10 mL) was added K₂CO₃ (390.6 mg, 2.81 mmol, 3 eq.) at room temperature under nitrogen atmosphere. The resulting mixture was stirred at room temperature for 12 h. The mixture was concentrated in vacuo and the residue was purified by reversed-phase flash chromatography with the following conditions: column, C18 silica gel; mobile phase, ACN in Water (0.5% TFA), 10% to 100% gradient in 20 min; detector, UV 254 nm. This resulted in tert-butyl 6-(2-{[(benzyloxy) carbonyl]amino}ethyl)-2,6-diazaspiro[3.4]octane-2-carboxylate (360 mg, 98.1%) as a colorless oil. MS: m/z: Calc'd for C₂₁H₃₁N₃O₄ [M+H]⁺390. Found, 390.

Step 2: To a stirred solution of tert-butyl 6-(2-{[(benzyloxy)carbonyl]amino}ethyl)-2,6-diazaspiro[3.4]octane-2-carboxylate (150 mg, 0.39 mmol, 1 eq.) in EtOH (40 mL) was added Pd/C (90.2 mg, 0.85 mmol, 2.2 eq.) in portions at room temperature under nitrogen atmosphere. The flask was evacuated and flushed three times with nitrogen, followed by flushing with hydrogen. The mixture was stirred 2h at room temperature under an atmosphere of hydrogen (balloon). After completion of reaction monitored by LCMS. The resulting mixture was filtered, the filter cake was washed with EtOH (3×30 mL). The filtrate was concentrated under reduced pressure. This resulted in tert-butyl 6-(2-aminoethyl)-2,6-diazaspiro[3.4]octane-2-carboxylate (90 mg, 91.8%) as a white solid. MS: m/z: Calc'd for C₁₃H₂₅N₃O₂ [M+H]⁺256. Found, 256.

Step 3: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.14 mmol, 1 eq.) and tert-butyl 6-(2-aminoethyl)-2,6-diazaspiro[3.4]octane-2-carboxylate (34.7 mg, 0.14 mmol, 1 eq.) in ACN (5 mL) was added DIEA (52.7 mg, 0.41 mmol, 3 eq.) at room temperature under nitrogen atmosphere. The resulting mixture was stirred at room temperature for 6 h. The reaction mixture was concentrated in vacuo and the resulting residue was purified by reversed-phase flash chromatography with the following conditions: column, C18 silica gel; mobile phase, ACN in Water (0.5% TFA), 10% to 60% gradient in 20 min; detector, UV 254 nm. This resulted in tert-butyl 6-{2-[({[(2R,3S,4S)-1-(tert-butoxycarbonyl)-4-[(tert-butoxycarbonyl)oxy]-2-[(4-methoxyphenyl)methyl]pyrrolidin-3-yl]oxy}carbonyl)amino]ethyl}-2,6-diazaspiro[3.4]octane-2-carboxylate (37 mg, 38.6%) as a light yellow oil. MS: m/z: Calc'd for: C₃₆H₅₆N₄O₁₀ [M+H]⁺705. Found, 705.

Step 4: To a stirred solution of tert-butyl 6-{2-[({[(2R,3S,4S)-1-(tert-butoxycarbonyl)-4-[(tert-butoxycarbonyl)oxy]-2-[(4-methoxyphenyl)methyl]pyrrolidin-3-yl]oxy}carbonyl)amino]ethyl}-2,6-diazaspiro[3.4]octane-2-carboxylate (34 mg, 0.05 mmol) in DCM (5 mL) was added TFA (1 mL) dropwise at room temperature under nitrogen atmosphere. The resulting mixture was stirred for 1.5 h. After completion of reaction monitored by LCMS, the mixture was concentrated. The crude product (30 mg) was purified by Prep-HPLC to afford (2R,3S,4S)-4-hydroxy-2-[(4-methoxyphenyl)methyl]pyrrolidin-3-yl N-(2-{2,6-diazaspiro[3.4]octan-6-yl}ethyl)carbamate; trifluoroacetic acid (20.7 mg, 81.4%) as a yellow semi-solid. MS: m/z: Calc'd for C₂₁H₃₂N₄O₄ [M+H]⁺405. Found, 405. ¹H NMR (400 MHz, Methanol-d₄) δ 7.25 (d, J=8.6 Hz, 2H), 6.94 (d, J=8.6 Hz, 2H), 5.02 (s, 1H), 4.43 (d, J=4.1 Hz, 1H), 4.27 (d, J=10.1 Hz, 2H), 4.15 (dd, J=18.8, 10.1 Hz, 3H), 3.80 (s, 3H), 3.64-3.48 (m, 4H), 3.37 (s, 5H), 3.20 (d, J=12.6 Hz, 1H), 3.11-3.09 (m, 1H), 2.97-2.95 (m, 1H), 2.53 (s, 2H). Prep-HPLC-conditions: Column: Welch Ultimate XB-C18 50*250, 10 μm; Mobile Phase A: Water (0.05% TFA), Mobile Phase B: ACN; Flow rate: 60 mL/min mL/min; Gradient: 2% B to 50% B in 20 min; Wave Length: 254 nm/220 nm; RT1(min): 16.

### Synthetic Procedure of V187652

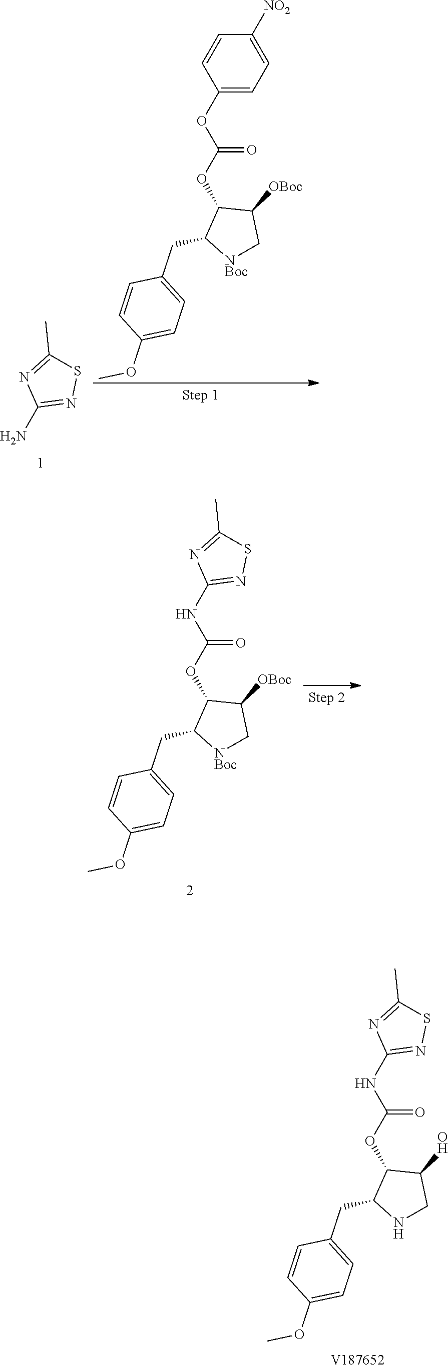

Step 1: To a stirred solution of 5-methyl-1,2,4-thiadiazol-3-amine (60 mg, 0.52 mmol, 1 eq.) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (306.7 mg, 0.52 mmol, 1 eq.) in ACN (8 mL) was added Cs₂CO₃ (509.3 mg, 1.56 mmol, 3 equiv), and the resulting mixture was stirred at room temperature for overnight. The reaction mixture was quenched with water and extracted with EA. The extracts were washed with brine, dried over anhydrous sodium sulfate, filtered and concentrated. The residue was purified by flash (10 mmol/L NH₄HCO₃ in water and acetonitrile) to afford tert-butyl (2R,3S,4S)-4-[(tert-butoxycarbonyl)oxy]-2-[(4-methoxyphenyl)methyl]-3-{[(5-methyl-1,2,4-thiadiazol-3-yl)carbamoyl]oxy}pyrrolidine-1-carboxylate (55 mg, 18.6%) as a white solid. MS: m/z: Calc'd for C₂₆H₃₆N₄O₈S [M+H−56−56]*453. found 453.

Step 2: To a stirred solution of tert-butyl (2R,3S,4S)-4-[(tert-butoxycarbonyl)oxy]-2-[(4-methoxyphenyl)methyl]-3-{[(5-methyl-1,2,4-thiadiazol-3-yl)carbamoyl]oxy}pyrrolidine-1-carboxylate (60 mg, 0.106 mmol, 1 equiv) in DCM (5 mL) was added TFA (1 mL) at room temperature, and the resulting mixture was stirred for 2 h. After completion of the reaction monitored by LCMS, the mixture was concentrated. The residue was purified by Prep-HPLC to obtain (2R,3S,4S)-4-hydroxy-2-[(4-methoxyphenyl)methyl]pyrrolidin-3-yl N-(5-methyl-1,2,4-thiadiazol-3-yl)carbamate; trifluoroacetic acid (26.0 mg, 50.89%) as a white solid. MS: m/z: Calc'd for C₁₆H₂₀N₄O₄S [M+H]⁺365. found 365. ¹H NMR (400 MHz, Methanol-d₄) δ 7.30-7.22 (m, 2H), 6.93 (d, J=8.4 Hz, 2H), 5.10 (d, J=3.5 Hz, 1H), 4.50 (d, J=4.1 Hz, 1H), 4.26 (dd, J=7.9, 3.5 Hz, 1H), 3.79 (s, 3H), 3.65 (dd, J=12.7, 4.3 Hz, 1H), 3.27 (d, J=12.6 Hz, 1H), 3.20 (dd, J=14.1, 7.2 Hz, 1H), 3.05 (dd, J=14.2, 8.5 Hz, 1H), 2.80 (s, 3H). Prep-HPLC-conditions: Column: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 5% B to 35% B in 10 min; Wave Length: 254 nm/220 nm; RT1(min): 8.68

### Synthetic Procedure of V187655

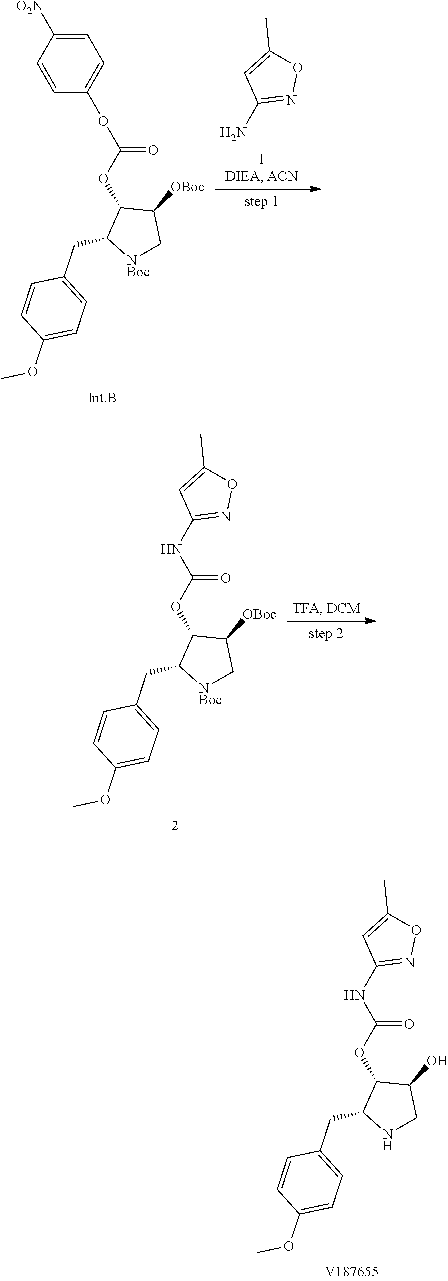

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 100 mg, 0.17 mmol, 1.0 eq.) and 5-methyl-1,2-oxazol-3-amine (33.3 mg, 0.34 mmol, 2.0 eq.) in MeCN (3 mL) was added DIEA (88 mg, 0.68 mmol, 4.0 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl) oxy]-2-[(4-methoxyphenyl)methyl]-3-{[(5-methyl-1,2-oxazol-3-yl)carbamoyl]oxy}pyrrolidine-1-carboxylate (70 mg, 75.2%) as a light yellow oil. MS: m/z: Calc'd for C₂₇H₃₇N₃O₉ [M+H−56−56]⁺436. Found, 436.

Step 2: To a stirred solution of tert-butyl (2R,3S,4S)-4-[(tert-butoxycarbonyl)oxy]-2-[(4-methoxyphenyl)methyl]-3-{[(5-methyl-1,2-oxazol-3-yl)carbamoyl]oxy}pyrrolidine-1-carboxylate (65 mg, 0.12 mmol, 1.0 eq.) in DCM (2 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-(5-methyl-1,2-oxazol-3-yl)carbamate; trifluoroacetic acid (15 mg, 27.2%) as a light yellow solid. MS: m/z: Calc'd for C₁₇H₂₁N₃O₅ [M+H]⁺348. Found, 348. ¹H NMR (400 MHz, Methanol-d₄) δ 7.29-7.21 (m, 2H), 6.97-6.89 (m, 2H), 6.48 (s, 1H), 5.07 (d, J=3.6 Hz, 1H), 4.48 (d, J=4.2 Hz, 1H), 4.30-4.20 (m, 1H), 3.79 (s, 3H), 3.62 (dd, J=12.6, 4.3 Hz, 1H), 3.26 (d, J=12.7 Hz, 1H), 3.17 (dd, J=14.2, 7.0 Hz, 1H), 3.02 (dd, J=14.2, 8.6 Hz, 1H), 2.42 (d, J=0.9 Hz, 3H). Prep-HPLC-conditions: Column: Xselect CSH Prep Fluoro-Phenyl Column, 19*250 mm, 5 μm; Mobile Phase A: Water (0.05% TFA), Mobile Phase B: MEOH; Flow rate: 25 mL/min mL/min; Gradient: 12% B to 42% B in 10 min; Wave Length: 254 nm/220 nm; RT1(min): 9.83

### Synthetic Procedure of V187657

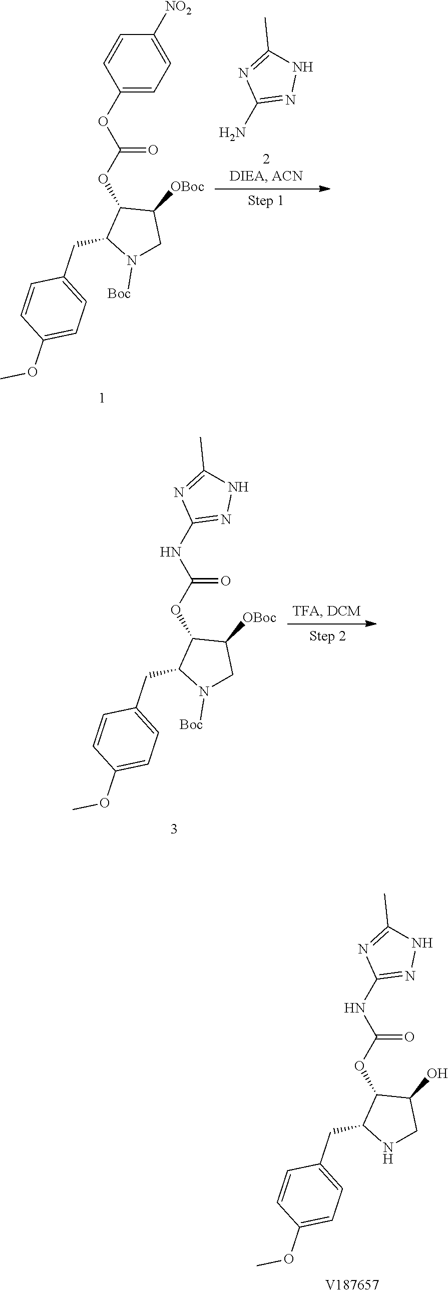

Step 1: To a solution of tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (80 mg, 0.14 mmol, 1 equiv), 5-methyl-1H-1,2,4-triazol-3-amine (16 mg, 0.16 mmol, 1.2 equiv) in MeCN (5 mL) was added DIEA (35 mg, 0.27 mmol, 2 equiv). The mixture was stirred for 2 h at room temperature under nitrogen atmosphere. Desired product could be detected by LCMS. The resulting mixture was concentrated under reduced pressure. The crude product was used in the next step directly without further purification. MS: m/z: Calc'd for C₂₆H₃₇N₅O₈ [M+H]⁺548. Found, 548.

Step 2: To the solution of tert-butyl (2R,3S,4S)-4-[(tert-butoxycarbonyl)oxy]-2-[(4-methoxy phenyl)methyl]-3-{[(5-methyl-1H-1,2,4-triazol-3-yl)carbamoyl]oxy}pyrrolidine-1-carboxylate (40 mg, 0.07 mmol, 1 equiv) in DCM (3 ml) was added TFA (1 mL) dropwise. The mixture was stirred for 2 h at room temperature under nitrogen atmosphere. The mixture was concentrated and the crude product (40 mg) was purified by Prep-HPLC to afford (2R,3S,4S)-4-hydroxy-2-[(4-methoxyphenyl)methyl]pyrrolidin-3-yl N-(5-methyl-1H-1,2,4-triazol-3-yl) carbamate (21.5 mg, 84.7%) as a white solid. MS: m/z: Calc'd for C₁₆H₂₁N₅O₄ [M+H]⁺348. Found, 348. ¹H NMR (400 MHz, DMSO-d₆) δ 7.27-7.16 (m, 2H), 6.89 (t, J=7.5 Hz, 2H), 5.03 (d, J=3.5 Hz, 1H), 4.46 (d, J=4.3 Hz, 1H), 4.12 (dd, J=8.1, 3.8 Hz, 1H), 3.75-3.69 (m, 3H), 3.51 (dd, J=12.8, 4.3 Hz, 1H), 3.15-2.87 (m, 3H), 2.18 (d, J=46.8 Hz, 3H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 25% B in 10 min; Wave Length: 254 nm/220 nm; RT1(min): 8.68

### Synthetic Procedure of V187658

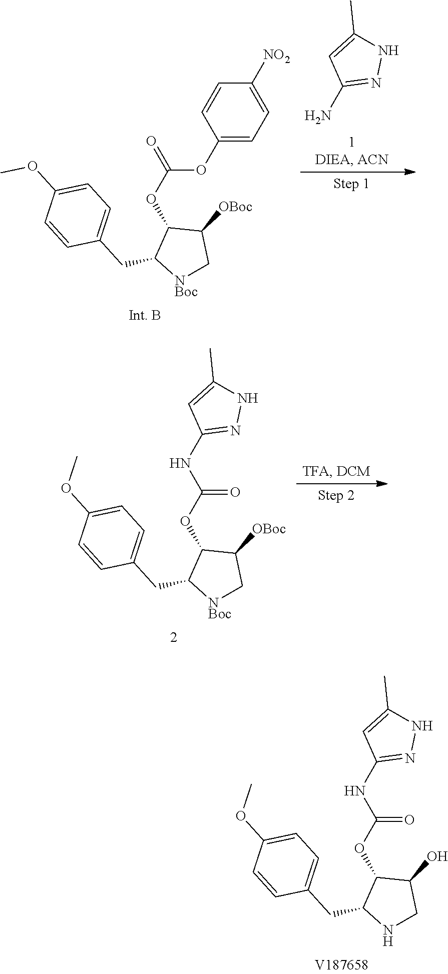

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (80 mg, 0.14 mmol, 1 eq.) and 5-methyl-1H-pyrazol-3-amine (13.2 mg, 0.14 mmol, 1 eq.) in ACN (5 mL) was added DIEA (52.7 mg, 0.41 mmol, 3 eq.) dropwise at room temperature under nitrogen atmosphere. The resulting mixture was stirred at room temperature for 12 h and was concentrated in vacuo. The residue was purified by reversed-phase flash chromatography with the following conditions: column, C18 silica gel; mobile phase, ACN in Water (0.5% TFA), 10% to 50% gradient in 20 min; detector, UV 254 nm. This resulted in tert-butyl (2R,3S,4S)-4-[(tert-butoxycarbonyl)oxy]-2-[(4-methoxyphenyl)methyl]-3-{[(5-methyl-1H-pyrazol-3-yl)carbamoyl]oxy}pyrrolidine-1-carboxylate (55 mg, 74.0%) as a colorless solid. MS: m/z: Calc'd for C₂₇H₃₈N₄O₈ [M+H]⁺657. Found, 657.

Step 2: To a stirred solution of tert-butyl (2R,3S,4S)-4-[(tert-butoxycarbonyl)oxy]-2-[(4-methoxyphenyl)methyl]-3-{[(5-methyl-1H-pyrazol-3-yl)carbamoyl]oxy}pyrrolidine-1-carboxylate (50 mg, 0.09 mmol) in DCM (5 mL) was added TFA (1 mL) dropwise at 0° C. under nitrogen atmosphere. The resulting mixture was stirred at room temperature for 2 h under nitrogen atmosphere. After completion of reaction monitored by LCMS, the mixture was concentrated. The crude product (40 mg) was purified by Prep-HPLC to afford (2R,3S,4S)-4-hydroxy-2-[(4-methoxyphenyl)methyl]pyrrolidin-3-yl N-(5-methyl-1H-pyrazol-3-yl)carbamate; trifluoroacetic acid (30.4 mg, 72.1%) as a white solid. MS: m/z: Calc'd for C₁₇H₂₂N₄O₄ [M+H]⁺347. Found, 347. ¹H NMR (400 MHz, Methanol-d₄) δ 7.25-7.17 (m, 2H), 6.97-6.90 (m, 2H), 5.19 (t, J=1.9 Hz, 1H), 4.51 (d, J=5.1 Hz, 1H), 4.22-4.23 (m, 1H), 3.79 (s, 4H), 3.27-3.18 (m, 1H), 3.18-3.06 (m, 2H), 2.23 (s, 3H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min m/min; Gradient: 3% B to 33% B in 10 min; Wave Length: 254 nm/220 nm; RT1(min): 8.68.

### Synthetic Procedure of V187659

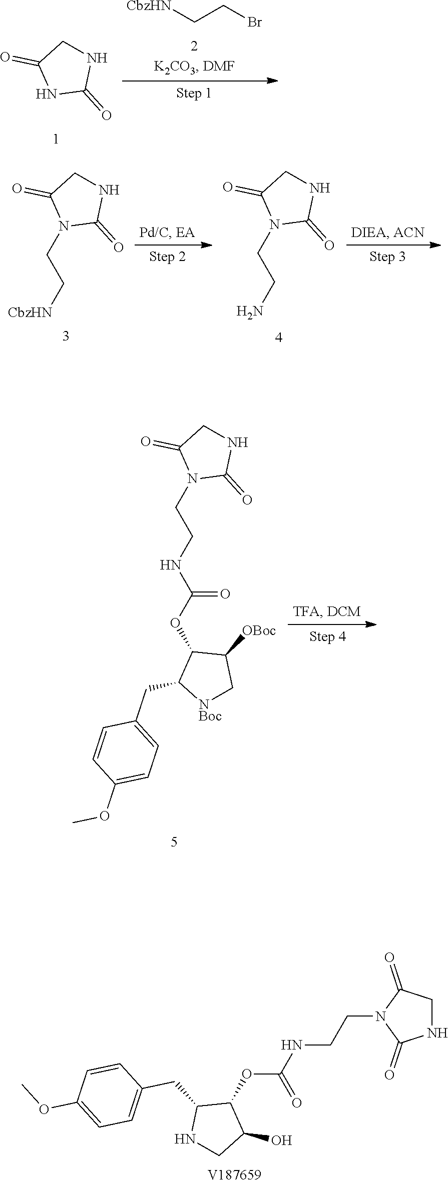

Step 1: To a stirred mixture of hydantoin (300 mg, 2.99 mmol, 1 equiv) and benzyl N-(2-bromoethyl)carbamate (696 mg, 2.69 mmol, 0.9 equiv) in ACN (5 mL) was added K₂CO₃ (1243 mg, 8.99 mmol, 3 equiv). The resulting mixture was stirred for overnight at 80° C. under air atmosphere. Desired product could be detected by LCMS. The mixture was filtered and the filtrate was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in benzyl N-[2-(2,5-dioxoimidazolidin-1-yl)ethyl]carbamate (100 mg, 10.7%) as a yellow oil. MS: m/z: Calc'd for C₁₃H₁₅N₃O₄ [M+H+22]⁺300. found 300.

Step 2: Under a nitrogen atmosphere, Pd/C (100 mg) was added to a solution of benzyl N-[2-(2,5-dioxoimidazolidin-1-yl)ethyl]carbamate (200 mg, 0.72 mmol, 1 equiv) in EtOH (10 mL), H2 was subsequently introduced into the reaction system, and the resulting mixture was stirred at r.t. for 2 h. Upon completion, the reaction mixture was filtered and concentrated in vacuo to afford crude product 3-(2-aminoethyl)imidazolidine-2,4-dione as a yellow oil.

Step 3: To a stirred mixture of 3-(2-aminoethyl)imidazolidine-2,4-dione (49 mg, 0.34 mmol, 2 equiv) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (100 mg, 0.17 mmol, 1.00 equiv) in ACN (5 mL) was added DIEA (66 mg, 0.51 mmol, 3 equiv). The resulting mixture was stirred for overnight at room temperature. Desired product could be detected by LCMS. The mixture was concentrated under vacuum. The residue was purified by reversed-phase flash chromatography with the following conditions: column, C18 silica gel; mobile phase, MeCN in Water (0.1% TFA), 10% to 50% gradient in 10 min; detector, UV 254 nm. This resulted in tert-butyl (2R,3S,4S)-4-[(tert-butoxycarbonyl)oxy]-3-({[2-(2,5-dioxoimidazolidin-1-yl)ethyl]carbamoyl}oxy)-2-[(4-methoxyphenyl)methyl]pyrrolidine-1-carboxylate (100 mg, 99.3%) as a yellow oil. MS: m/z: Calc'd for C₅H₉N₃O₂[M+H−100−56]⁺437. found 437.

Step 4: To a stirred solution of tert-butyl (2R,3S,4S)-4-[(tert-butoxycarbonyl)oxy]-3-({[2-(2,5-dioxoimidazolidin-1-yl)ethyl]carbamoyl}oxy)-2-[(4-methoxyphenyl)methyl]pyrrolidine-1-carboxylate (80 mg, 0.13 mmol, 1 equiv) in DCM (5 mL) was added TFA (1 mL) dropwise at 0° C. The resulting mixture was stirred for 2 h at room temperature under air atmosphere. Desired product could be detected by LCMS. The resulting mixture was concentrated under vacuum. The crude product (50 mg) was purified by Prep-HPLC to afford (2R,3S,4S)-4-hydroxy-2-[(4-methoxyphenyl)methyl]pyrrolidin-3-yl N-[2-(2,5-dioxoimidazolidin-1-yl)ethyl]carbamate; trifluoroacetic acid (37.6 mg, 54.7%) as a white solid. MS: m/z: Calc'd for C₁₈H₂₄N₄O₆ [M+H]⁺393. found 393. ¹H NMR (400 MHz, Methanol-d₄) δ 7.31-7.20 (m, 2H), 6.97-6.89 (m, 2H), 4.90 (s, 1H), 4.43 (dd, J=3.9, 1.4 Hz, 1H), 4.13-4.05 (m, 1H), 3.94 (s, 2H), 3.79 (s, 3H), 3.64-3.52 (m, 3H), 3.46-3.33 (m, 2H), 3.25-2.88 (m, 3H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 24% B in 10 min; Wave Length: 254 nm/220 nm; RT1(min): 8.68

### Synthetic Procedure of V187660

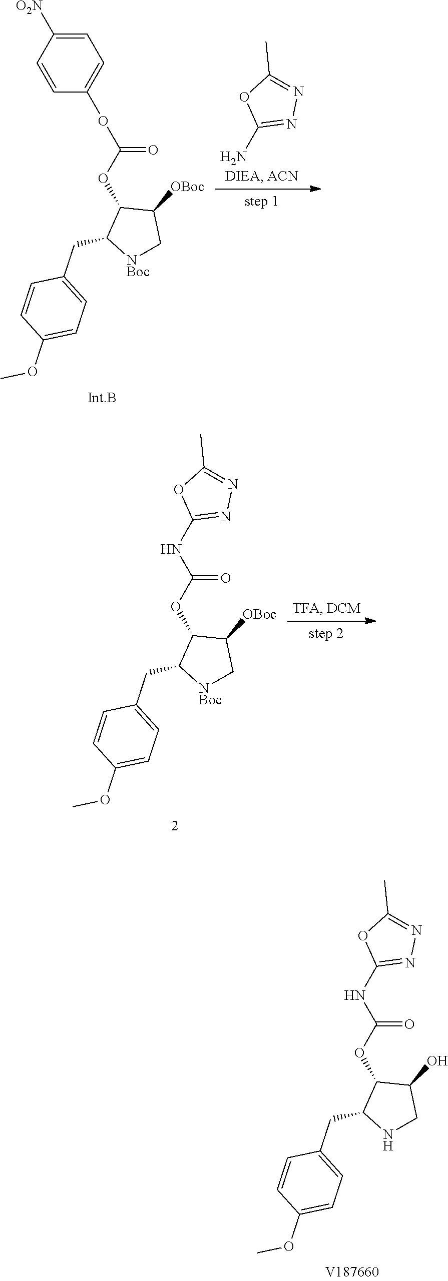

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.14 mmol, 1.0 eq) and 2-amino-5-ME-1,3,4-oxadiazole (26.9 mg, 0.27 mmol, 2.0 eq) in ACN (3 mL) was added Cs₂CO₃ (132.9 mg, 0.41 mmol, 3.0 eq) in portions at room temperature. The resulting mixture was stirred at ambient temperature for overnight. After completion of the reaction monitored by LCMS. The residue was purified by reversed-phase flash chromatography with the following conditions: column, C18 silica gel; mobile phase, MeCN in Water (0.1% TFA), 10% to 50% gradient in 10 min; detector, UV 220 nm to afford tert-butyl (2R,3S,4S)-4-[(tert-butoxycarbonyl)oxy]-2-[(4-methoxyphenyl)methyl]-3-{[(5-methyl-1,3,4-oxadiazol-2-yl)carbamoyl]oxy}pyrrolidine-1-carboxylate (65 mg, 87.2%) as a light yellow oil. MS: m/z: Calc'd for C₂₆H₃₆N₄O₉ [M+H]⁺549. Found, 549.

Step 2: To a stirred solution of tert-butyl (2R,3S,4S)-4-[(tert-butoxycarbonyl)oxy]-2-[(4-methoxyphenyl)methyl]-3-{[(5-methyl-1,3,4-oxadiazol-2-yl)carbamoyl]oxy}pyrrolidine-1-carboxylate (60 mg, 0.11 mmol) in DCM (2 mL, 31.46 mmol) was added TFA (1 mL) at room temperature. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl)methyl]pyrrolidin-3-yl N-(5-methyl-1,3,4-oxadiazol-2-yl)carbamate; trifluoroacetic acid (19.9 mg, 39.2%) as a white solid. MS: m/z: Calc'd for C₁₆H₂₀N₄O₅ [M+H]⁺349. Found, 349. ¹H NMR (400 MHz, Methanol-d₄) δ 7.27-7.23 (m, 2H), 6.93-6.91 (m, 2H), 5.09 (d, J=3.6 Hz, 1H), 4.50 (d, J=3.6 Hz, 1H), 4.28-4.23 (m, 1H), 3.79 (d, J=3.6 Hz, 3H), 3.66-3.60 (m, 1H), 3.26-3.24 (m, 1H), 3.18-3.16 (m, 1H), 3.09-3.00 (m, 1H), 2.50 (d, J=4.3 Hz, 3H). Prep-HPLC-conditions: (Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 25% B in 10 min; Wave Length: 254 nm/220 nm; RT1(min): 8.68.

### Synthetic Procedure of V187666

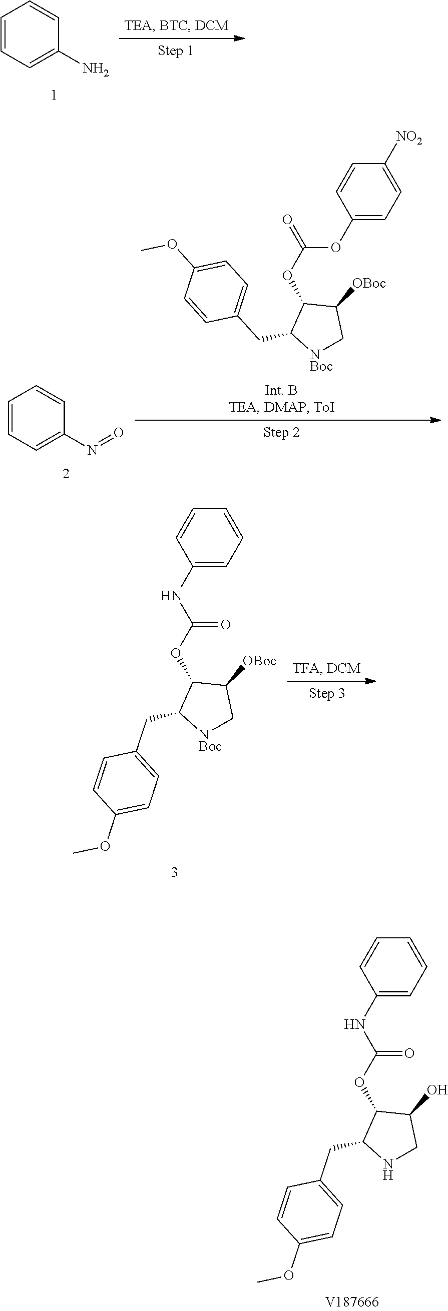

Step 1: To a stirred solution of aniline (200 mg, 2.14 mmol, 1 eq.) and ditrichloromethyl carbonate (637.2 mg, 2.14 mmol, 1 eq.) in DCM (4 mL) were added TEA (651.9 mg, 6.44 mmol, 3 eq.) at 0° C. The reaction was stirred at 0° C. for 2 h under nitrogen atmosphere. After completion of the reaction monitored by LCMS. The mixture was concentrated to give the crude product, which was used in the next step without further purification. MS: m/z: Calc'd for C₁₄H₁₄N₂O[M+H]⁺227. Found, 227.

Step 2: To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (Int.B, 95 mg, 0.01 mmol, 1 eq.) and phenyl isocyanate (53.4 mg, 0.44 mmol, 2 eq.) in Toluene (3 mL) were added TEA (68.1 mg, 0.67 mmol, 3 eq.) and DMAP (27.4 mg, 0.22 mmol, 1 eq.) at 0° C. The reaction was stirred at r.t. for overnight. The reaction mixture was quenched with water and extracted with EA. The extracts were washed with brine, dried over anhydrous sodium sulfate, filtered and concentrated. The residue was purified by flash to afford tert-butyl (2R,3S,4S)-4-[(tert-butoxycarbonyl)oxy]-2-[(4-methoxyphenyl)methyl]-3-[(phenylcarbamoyl)oxy]pyrrolidine-1-carboxylate (80 mg, 65.7%) as a white solid. MS: m/z: Calc'd for C₂₉H₃₈N₂O₈[M−H]⁻541. Found, 541.

Step 3: To a stirred solution of tert-butyl (2R,3S,4S)-4-[(tert-butoxycarbonyl)oxy]-2-[(4-methoxyphenyl)methyl]-3-[(phenylcarbamoyl)oxy]pyrrolidine-1-carboxylate (75 mg, 0.13 mmol, 1 eq.) in DCM (3 mL) was added TFA (1 mL) dropwise at 0° C. The reaction was stirred at r.t. for 2h. After completion of the reaction monitored by LCMS, the mixture was concentrated. The crude product (70 mg) was purified by Prep-HPLC to afford (2R,3S,4S)-4-hydroxy-2-[(4-methoxyphenyl)methyl]pyrrolidin-3-yl N-phenylcarbamate (23.3 mg, 47.3%) as a white solid. MS: m/z: Calc'd for C₁₉H₂₂N₂O₄ [M+H]⁺343. Found, 343. ¹H NMR (400 MHz, Methanol-d₄) δ 7.48 (d, J=8.0 Hz, 2H), 7.38-7.29 (m, 2H), 7.26 (d, J=8.5 Hz, 2H), 7.10 (t, J=7.5 Hz, 1H), 6.97-6.89 (m, 2H), 5.10-5.04 (m, 1H), 4.49 (d, J=4.1 Hz, 1H), 4.24-4.20 (m, 1H), 3.79 (s, 3H), 3.63-3.59 (m, 1H), 3.26-3.19 (m, 2H), 3.03-2.98 (m, 1H). Prep-HPLC-conditions: Xselect CSH Prep C18 Column, 30*150 mm, 5 μm; Mobile Phase A: Water (0.05% TFA), Mobile Phase B: ACN; Flow rate: 60 mL/min mL/min; Gradient: 10% B to 40% B in 10 min; Wave Length: 254 nm/220 nm; RT1(min): 8.5

### Synthetic Procedure of V187668

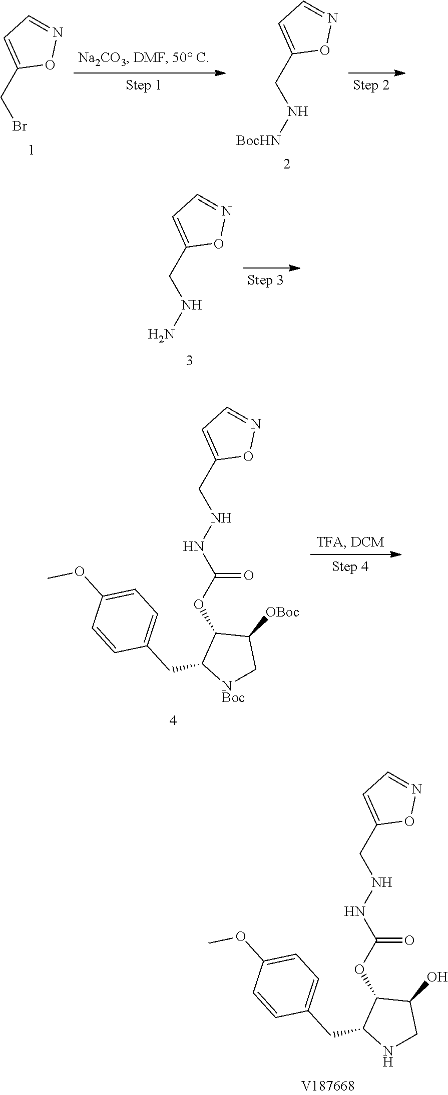

Step 1: To a stirred solution of 5-(bromomethyl)-1,2-oxazole (300 mg, 1.85 mmol, 1 eq.) and tert-butoxycarbohydrazide (489.5 mg, 3.70 mmol, 2 eq.) in DMF (10 mL) was added Na₂CO₃ (785.1 mg, 7.41 mmol, 4 eq.), and the resulting mixture was stirred at 50° C. for overnight. After completion of the reaction monitored by LCMS, the mixture was purified by reverse-phase column to obtain N′-(1,2-oxazol-5-ylmethyl)tert-butoxycarbohydrazide (210 mg, 53.1%) as a white solid. MS: m/z: Calc'd for C₉H₁₅N₃O₃ [M−H]⁺212. found 212.

Step 2: To a stirred solution of N′-(1,2-oxazol-5-ylmethyl)tert-butoxycarbohydrazide (210 mg, 0.98 mmol, 1 eq.) in DCM (10 mL) was added TFA (2 mL), and the resulting mixture was stirred at room temperature for 2 h. LCMS showed the reaction was completed. The mixture was concentrated to obtain 5-(hydrazinylmethyl)-1,2-oxazole (110 mg, 98.7%) as a yellow oil. MS: m/z: Calc'd for C₄H₇N₃O [M+H]⁺114. found 114.

Step 3: To a stirred solution of 5-(hydrazinylmethyl)-1,2-oxazole (100 mg, 0.88 mmol, 1 eq.) and tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (520.3 mg, 0.88 mmol, 1 eq.) in ACN (10 mL) was added DIEA (342.7 mg, 2.65 mmol, 3 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl)oxy]-2-[(4-methoxyphenyl)methyl]-3-{[N′-(1,2-oxazol-5-ylmethyl)hydrazinecarbonyl]oxy}pyrrolidine-1-carboxylate (100 mg, 20.1%) as a white solid. MS: m/z: Calc'd for C₂₇H₃₈N₄O₉ [M+H−56−100]⁺407. found 407.

Step 4: To a stirred solution of tert-butyl (2R,3S,4S)-4-[(tert-butoxycarbonyl)oxy]-2-[(4-methoxyphenyl)methyl]-3-{[N′-(1,2-oxazol-5-ylmethyl)hydrazinecarbonyl]oxy}pyrrolidine-1-carboxylate (60 mg, 0.11 mmol, 1 eq.) in DCM (5 mL) was added TFA (1 mL), and the resulting mixture was stirred at room temperature for 2 h. After completion of the reaction monitored by LCMS, the mixture was concentrated. The residue was purified by Prep-HPLC to obtain 1-{[(2R,3S,4S)-4-hydroxy-2-[(4-methoxyphenyl)methyl]pyrrolidin-3-yl]oxy}-N′-(1,2-oxazol-5-ylmethyl)formohydrazide; trifluoroacetic acid (21.0 mg, 41.29%) as a light-yellow semi-solid. MS: m/z: Calc'd for C₁₇H₂₂N₄O₅ [M+H]⁺363. found 363. ¹H NMR (400 MHz, Methanol-d₄) δ 8.36 (s, 1H), 7.25-7.17 (m, 2H), 6.93 (dd, J=8.5, 3.1 Hz, 2H), 6.43 (s, 1H), 4.99-4.95 (m, 1H), 4.39 (s, 1H), 4.19 (d, J=1.6 Hz, 3H), 3.80 (s, 3H), 3.55 (d, J=12.9 Hz, 1H), 3.22 (d, J=12.5 Hz, 1H), 3.08 (dd, J=14.0, 7.2 Hz, 1H), 2.94 (dd, J=14.3, 8.5 Hz, 1H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 25% B in 10 min; Wave Length: 254 nm/220 nm; RT1(min): 8.68

### Synthetic Procedure of V187681

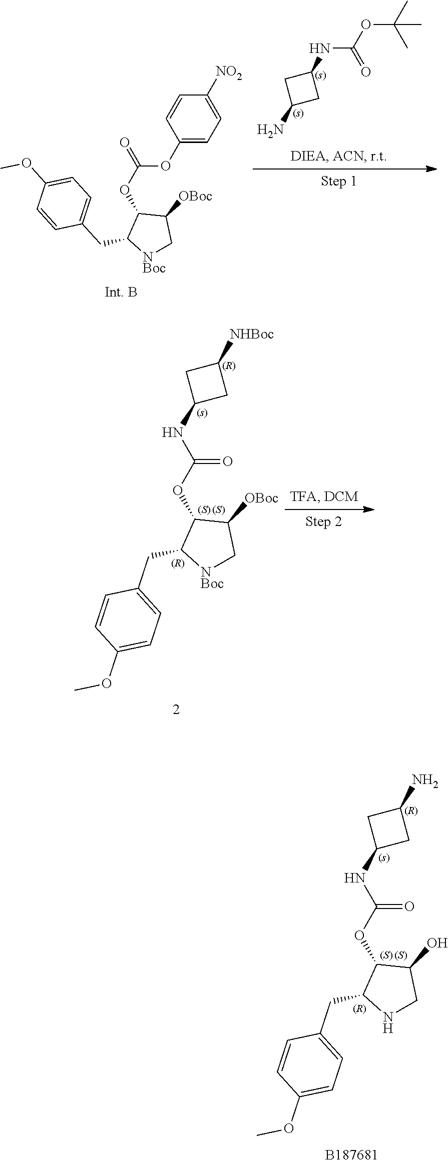

Step 1: To a solution of tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (80 mg, 0.13 mmol, 1 equiv), tert-butyl N-[(1s,3s)-3-aminocyclobutyl]carbamate (30 mg, 0.16 mmol, 1.2 equiv) in MeCN (5 ml) was added DIEA (35 mg, 0.27 mmol, 2 equiv), the mixture was stirred for 4 h at room temperature under nitrogen atmosphere. Desired product could be detected by LCMS. The mixture was concentrated and the residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. afford tert-butyl (2R,3S,4S)-4-[(tert-butoxycarbonyl)oxy]-2-[(4-methoxyphenyl)methyl]-3-({[(1s,3s)-3-[(tert-butoxy carbonyl)amino]cyclobutyl]carbamoyl}oxy)pyrrolidine-1-carboxylate (60 mg, 69.4%) as a yellow oil. MS: m/z: Calc'd for C₃₂H₄₉N₃O₁₀ [M−H]-634. Found, 634.

Step 2: A solution of tert-butyl (2R,3S,4S)-4-[(tert-butoxycarbonyl)oxy]-2-[(4-methoxy phenyl)methyl]-3-({[(1s,3s)-3-[(tert-butoxycarbonyl)amino]cyclobutyl]carbamoyl}oxy) pyrrolidine-1-carboxylate (45 mg, 0.07 mmol, 1 equiv) in DCM (3 mL) was added TFA (1 mL) dropwise. The mixture was stirred at room temperature for 2 h under N₂ atmosphere. Desired product could be detected by LCMS. The mixture was concentrated to give the crude product. The crude product (30 mg) was purified by Prep-HPLC to afford (2R,3S,4S)-4-hydroxy-2-[(4-methoxyphenyl)methyl]pyrrolidin-3-yl N-[(1s,3s)-3-aminocyclobutyl]carbamate (24.2 mg, 98%) as a white solid. MS: m/z: Calc'd for C₁₇H₂₅N₃O₄ [M+H]⁺336. Found, 336. ¹H NMR (400 MHz, Methanol-d₄) δ 7.14 (d, J=8.3 Hz, 2H), 6.89-6.79 (m, 2H), 4.66 (s, 1H), 4.18-4.12 (m, 1H), 3.78 (s, 4H), 3.62-3.58 (m, 1H), 3.23 (t, J=8.1 Hz, 1H), 2.89-2.66 (m, 5H), 1.93-1.75 (m, 2H). Prep-HPLC-conditions: Column: Welch Ultimate XB-C18 50*250, 10 μm; Mobile Phase A: Water (10 nmol/LNH4HCO3), Mobile Phase B: ACN; Flow rate: 60 mL/min mL/min; Gradient: 1% B to 50% B in 20 min; Wave Length: 254 nm/220 nm; RT1(min): 16

### Synthetic Procedure of V187682

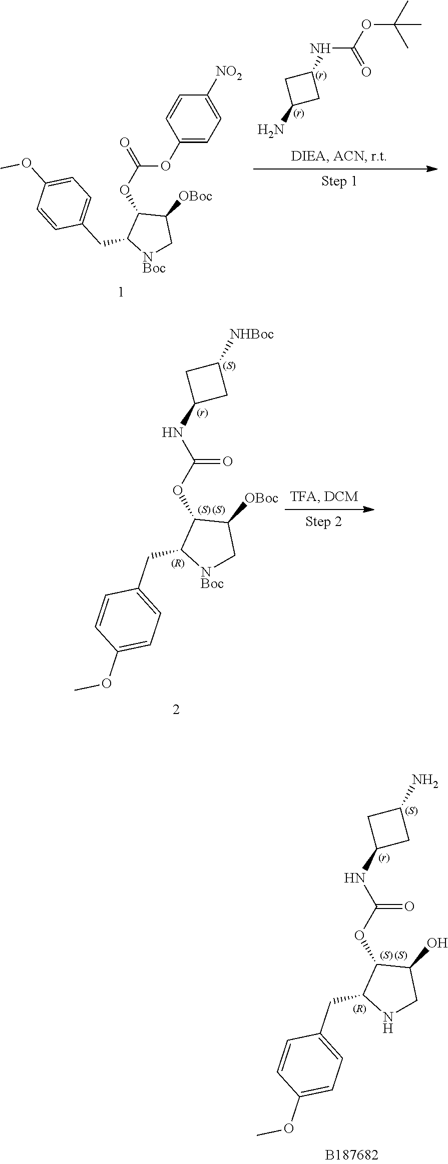

Step 1: To a solution of tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (80 mg, 0.14 mmol, 1 equiv), tert-butyl N-[(1r,3r)-3-aminocyclobutyl]carbamate (30.4 mg, 0.16 mmol, 1.2 equiv) in MeCN (5 mL) was added DIEA (35 mg, 0.27 mmol, 2 equiv), the mixture was stirred for 2 h at room temperature under nitrogen atmosphere. Desired product could be detected by LCMS. The mixture was concentrated. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 220 nm. To afford tert-butyl (2R,3S,4S)-4-[(tert-butoxycarbonyl)oxy]-2-[(4-methoxyphenyl)methyl]-3-({[(1r,3r)-3-[(tert-butoxycarbonyl)amino]cyclobutyl]carbamoyl}oxy)pyrrolidine-1-carboxylate (55 mg, 63.6%) as a yellow oil. MS: m/z: Calc'd for C₃₂H₄₉N₃O₁₀ [M−H]-634. Found, 634.

Step 2: To a solution of tert-butyl (2R,3S,4S)-4-[(tert-butoxycarbonyl)oxy]-2-[(4-methoxy phenyl)methyl]-3-({[(1r,3r)-3-[(tert-butoxycarbonyl)amino]cyclobutyl]carbamoyl}oxy) pyrrolidine-1-carboxylate (50 mg, 0.08 mmol, 1 equiv) in DCM (3 mL) was added TFA (1 mL). The mixture was stirred for 2 h at room temperature under N₂. Desired product could be detected by LCMS. The mixture was concentrate to give the crude product. The crude product (30 mg) was purified by Prep-HPLC to afford (2R,3S,4S)-4-hydroxy-2-[(4-methoxyphenyl)methyl]pyrrolidin-3-yl N-[(1r,3r)-3-aminocyclobutyl]carbamate (12 mg, 45.5%) as a white solid. MS: m/z: Calc'd for C₁₇H₂₅N₃O₄ [M+H]⁺336. Found, 336. ¹H NMR (400 MHz, Methanol-d₄) δ 7.14 (d, J=8.1 Hz, 2H), 6.85 (d, J=8.1 Hz, 2H), 4.63 (t, J=2.4 Hz, 1H), 4.21-4.11 (m, 2H), 3.77 (d, J=1.4 Hz, 3H), 3.66 (t, J=7.0 Hz, 1H), 3.55 (d, J=5.0 Hz, 1H), 2.88-2.72 (m, 3H), 2.28-2.10 (m, 4H). Prep-HPLC-conditions: Column: Welch Ultimate XB-C18 50*250, 10 μm; Mobile Phase A: Water (10 nmol/L NH₄HCO₃), Mobile Phase B: ACN; Flow rate: 60 mL/min mL/min; Gradient: 1% B to 50% B in 20 min; Wave Length: 254 nm/220 nm; RT1(min): 17

### Synthetic Procedure of V187683

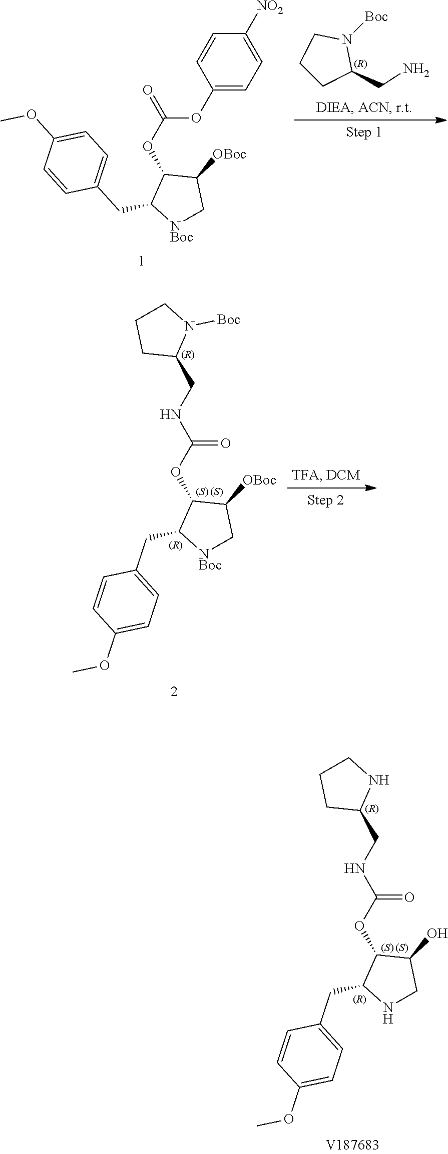

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (80 mg, 0.14 mmol, 1 eq) and tert-butyl (2R)-2-(aminomethyl)pyrrolidine-1-carboxylate (40.8 mg, 0.20 mmol, 1.5 eq) in ACN (5 mL) was added DIEA (70.2 mg, 0.54 mmol, 4 eq). The resulting reaction mixture was stirred at ambient temperature for overnight. After completion of the reaction monitored by LCMS, the mixture was purified directly by reversed-phase column chromatography (6.5 mmol/L NH₄HCO₃ in water and acetonitrile) to afford tert-butyl (2R,3S,4S)-4-[(tert-butoxycarbonyl)oxy]-3-[({[(2R)-1-(tert-butoxycarbonyl)pyrrolidin-2-yl]methyl}carbamoyl)oxy]-2-[(4-methoxyphenyl)methyl]pyrrolidine-1-carboxylate (75 mg, 84.9%) as an off-white solid. MS: m/z: Calc'd for C₃₃H₅₁N₃O₁₀ [M+H−100]⁺ 550. found 550.

Step 2: To a solution of tert-butyl (2R,3S,4S)-4-[(tert-butoxycarbonyl)oxy]-3-[({[(2R)-1-(tert-butoxycarbonyl)pyrrolidin-2-yl]methyl}carbamoyl)oxy]-2-[(4-methoxyphenyl)methyl]pyrrolidine-1-carboxylate (70 mg, 0.11 mmol) in DCM (5 mL) was added TFA (1 mL), the mixture was stirred at RT for 1 h. LCMS showed the starting material was consumed completely. The resulting solution was diluted with 10 mL of DCM and directly concentrated in vacuo to obtain the crude, which was purified by reversed-phase column chromatography (0.05% TFA and MeCN) and further purified by Prep-HPLC to obtain (2R,3S,4S)-4-hydroxy-2-[(4-methoxy phenyl)methyl]pyrrolidin-3-yl N-[(2R)-pyrrolidin-2-ylmethyl]carbamate (18.6 mg, 48.7%) as a white solid. MS: m/z: Calc'd for C₁₈H₂₇N₃O₄ [M+H]⁺350. found 350. ¹H NMR (400 MHz, Methanol-d₄) δ 7.25 (d, J=8.6 Hz, 2H), 6.96-6.90 (m, 2H), 5.00 (d, J=4.3 Hz, 1H), 4.44 (d, J=4.2 Hz, 1H), 4.23-4.11 (m, 1H), 3.80 (s, 3H), 3.78-3.68 (m, 1H), 3.59 (dd, J=12.7, 4.3 Hz, 1H), 3.49-3.47 (m, 2H), 3.42-3.35 (m, 1H), 3.32-3.27 (m, 1H), 3.22 (d, J=12.6 Hz, 1H), 3.12-3.10 (m, 1H), 3.00-2.98 (m, 1H), 2.35-1.93 (m, 3H), 1.89-1.71 (m, 1H). Prep-HPLC conditions: Column: Xselect CSH Prep C18 OBD Column, 19*250 mm, 5 μm; Mobile Phase A: Water (0.05% TFA), Mobile Phase B: ACN; Flow rate: 25 mL/min mL/min; Gradient: 3% B to 19% B in 6 min; Wave Length: 254 nm/220 nm; RT1(min): 5.08

### Synthetic Procedure of V187684

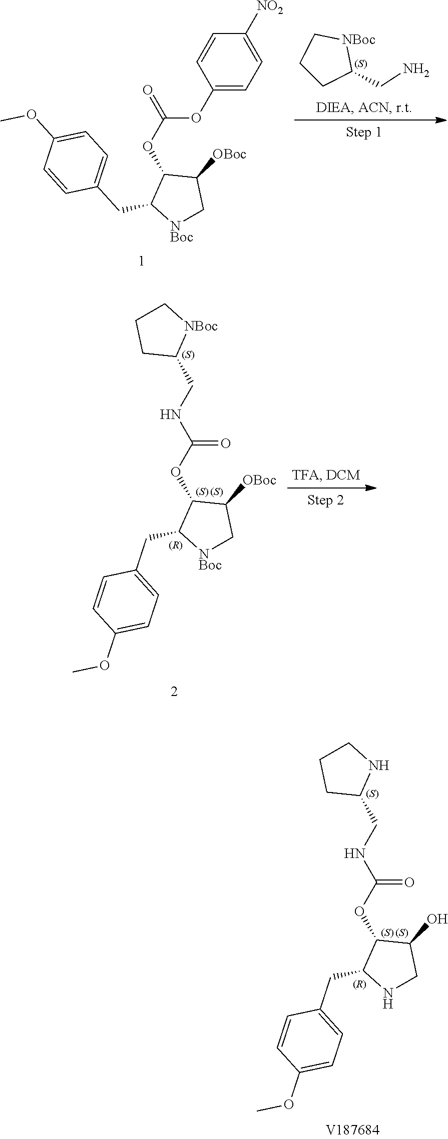

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (80 mg, 0.14 mmol, 1 eq) and tert-butyl (2S)-2-(aminomethyl)pyrrolidine-1-carboxylate (40.8 mg, 0.20 mmol, 1.5 eq) in ACN (5 mL) was added DIEA (70.3 mg, 0.54 mmol, 4 eq). The resulting reaction mixture was stirred at ambient temperature for overnight. After completion of the reaction monitored by LCMS, the mixture was purified directly by reversed-phase column chromatography (6.5 mmol/L NH₄HCO₃ in water and acetonitrile) to afford tert-butyl (2R,3S,4S)-4-[(tert-butoxycarbonyl)oxy]-3-[({[(2S)-1-(tert-butoxycarbonyl)pyrrolidin-2-yl]methyl}carbamoyl)oxy]-2-[(4-methoxyphenyl)methyl]pyrrolidine-1-carboxylate (75 mg, 84.9%) as an off-white solid. MS: m/z: Calc'd for C₃₃H₅₁N₃O₁₀ [M+H]⁺650. found 650.

Step 2: To a solution of tert-butyl (2R,3S,4S)-4-[(tert-butoxycarbonyl)oxy]-3-[({[(2S)-1-(tert-butoxycarbonyl)pyrrolidin-2-yl]methyl}carbamoyl)oxy]-2-[(4-methoxyphenyl)methyl]pyrrolidine-1-carboxylate (70 mg, 0.11 mmol) in DCM (5 mL) was added TFA (1 mL), the mixture was stirred at RT for 1 h. LCMS showed the starting material was consumed completely. The resulting solution was diluted with 10 mL of DCM and directly concentrated in vacuo to obtain the crude, which was purified by reversed-phase column chromatography (0.05% TFA and MeCN) and further purified by Prep-HPLC to obtain (2R,3S,4S)-4-hydroxy-2-[(4-methoxy phenyl)methyl]pyrrolidin-3-yl N-[(2S)-pyrrolidin-2-ylmethyl]carbamate; trifluoroacetic acid (20.0 mg, 39.5%) as a white solid. MS: m/z: Calc'd for C₁₈H₂₇N₃O₄ [M+H]⁺350. found 350.

¹H NMR (400 MHz, Methanol-d₄) δ 7.25 (d, J=8.1 Hz, 2H), 6.97-6.90 (m, 2H), 5.04-4.99 (m, 1H), 4.43 (d, J=4.1 Hz, 1H), 4.24-4.14 (m, 1H), 3.80 (d, J=2.1 Hz, 4H), 3.65-3.44 (m, 3H), 3.37 (t, J=7.6 Hz, 1H), 3.32 (s, 1H), 3.22 (d, J=12.6 Hz, 1H), 3.13 (dd, J=14.4, 6.4 Hz, 1H), 2.99 (dd, J=14.3, 9.1 Hz, 1H), 2.31-2.02 (m, 3H), 1.91-1.73 (m, 1H). Prep-HPLC conditions: Column: Xselect CSH Prep C18 OBD Column, 19*250 mm, 5 μm; Mobile Phase A: Water (0.05% TFA), Mobile Phase B: ACN; Flow rate: 25 mL/min mL/min; Gradient: 3% B to 19% B in 5.5 min; Wave Length: 254 nm/220 nm; RT1(min): 4.85

### Synthetic Procedure of V187685

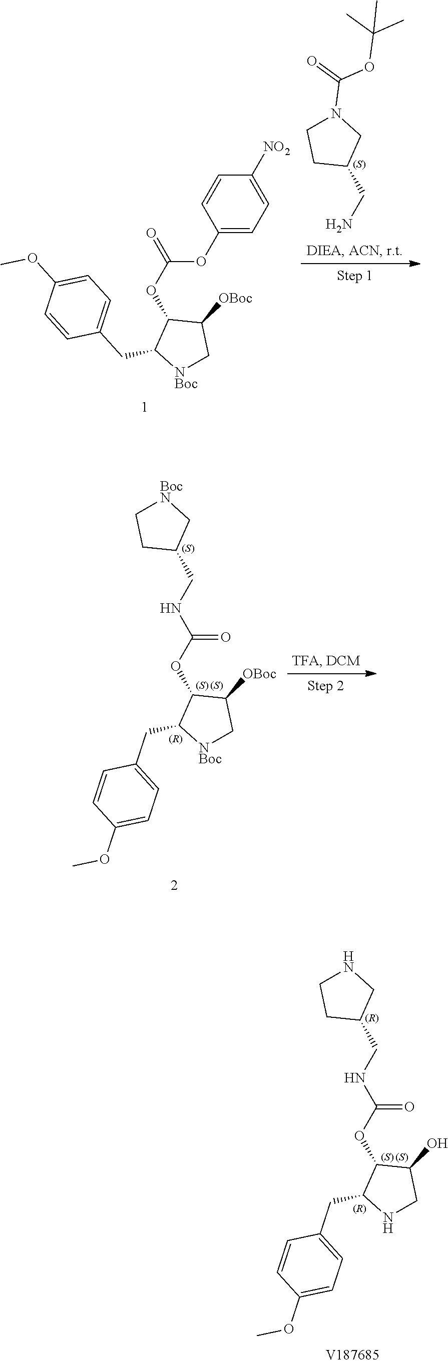

Step 1: To a solution of tert-butyl (2R,3S,4S)-4-[(tert-butoxycarbonyl)oxy]-2-[(4-methoxy phenyl)methyl]-3-[(4-nitrophenoxycarbonyl)oxy]pyrrolidine-1-carboxylate (80 mg, 0.14 mmol, 1 equiv), tert-butyl (3S)-3-(aminomethyl)pyrrolidine-1-carboxylate (33 mg, 0.16 mmol, 1.2 equiv) in MeCN (5 mL) was added and DIEA (35 mg, 0.27 mmol, 2 equiv), the mixture was stirred for 2 h at room temperature under nitrogen atmosphere. Desired product could be detected by LCMS. The mixture was concentrated to give the crude product which was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm afford tert-butyl (2R,3S,4S)-4-[(tert-butoxycarbonyl)oxy]-3-[({[(3S)-1-(tert-butoxycarbonyl)pyrrolidin-3-yl]methyl}carbamoyl)oxy]-2-[(4-methoxyphenyl)methyl]pyrrolidine-1-carboxylate (74 mg, 83.8%) as a yellow oil. MS: m/z: Calc'd for C₁₈H₂₅N₃O₂ [M+H]⁺650. Found, 650.

Step 2: To a solution of tert-butyl (2R,3S,4S)-4-[(tert-butoxycarbonyl)oxy]-3-[({[(3S)-1-(tert-butoxycarbonyl)pyrrolidin-3-yl]methyl}carbamoyl)oxy]-2-[(4-methoxyphenyl)methyl]pyrrolidine-1-carboxylate (60 mg, 0.09 mmol) in DCM (3 mL) was added TFA (1 mL) dropwise. The mixture was stirred at room temperature for 2 hours under nitrogen atmosphere. Desired product could be detected by LCMS. The mixture was concentrated to give the crude product (40 mg) which was purified by Prep-HPLC to afford (2R,3S,4S)-4-hydroxy-2-[(4-methoxy phenyl)methyl]pyrrolidin-3-yl N-[(3R)-pyrrolidin-3-ylmethyl]carbamate (17.4 mg, 52.5%) as a white solid. MS: m/z: Calc'd for C₃₀H₄₂N₄O₄ [M+H]⁺350. Found, 350. ¹H NMR (400 MHz, Methanol-d₄) δ 7.17-7.10 (m, 2H), 6.88-6.81 (m, 2H), 4.62 (d, J=3.9 Hz, 1H), 4.14-4.08 (m, 1H), 3.77 (s, 3H), 3.50-3.45 (m, 1H), 3.30 (d, J=5.7 Hz, 1H), 3.24-2.94 (m, 5H), 2.88-2.67 (m, 4H), 2.49-2.32 (m, 1H), 2.02-1.98 (m, 1H), 1.72-1.51 (m, 1H). Prep-HPLC-conditions: Column: XBridge Prep OBD C18 Column, 30*150 mm, 5 μm; Mobile Phase A: Water (10 mmol/L TFA), Mobile Phase B: ACN; Flow rate: 60 mL/min mL/min; Gradient: 2% B to 21% B in 13 min; Wave Length: 254 nm/220 nm; RT1(min): 7.84

### Synthetic Procedure of V187687

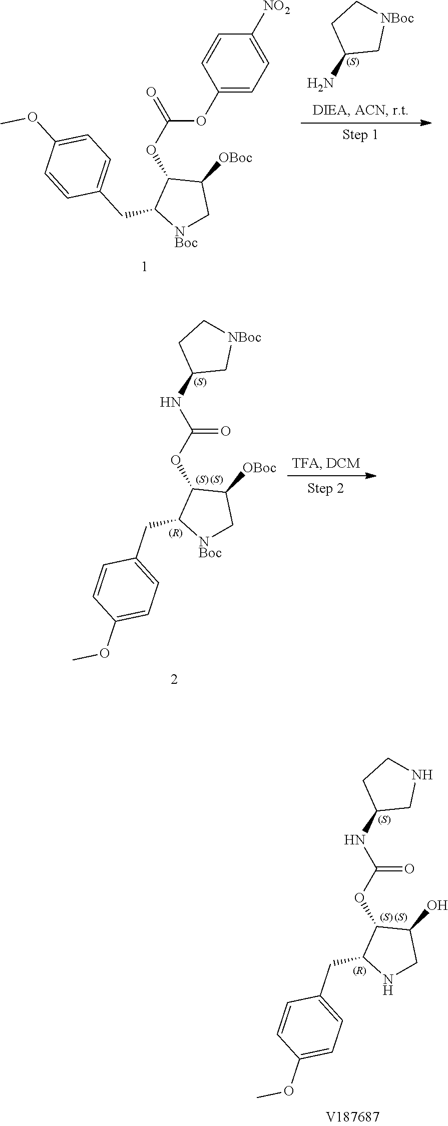

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (80 mg, 0.14 mmol, 1 eq.) and tert-butyl (3S)-3-aminopyrrolidine-1-carboxylate (37.9 mg, 0.20 mmol, 1.5 eq.) in ACN (5 mL) was added DIEA (70.3 mg, 0.54 mmol, 4 eq.). The resulting reaction mixture was stirred at ambient temperature for overnight. After completion of the reaction monitored by LCMS, the mixture was purified directly by reversed-phase column chromatography (6.5 mmol/L NH₄HCO₃ in water and acetonitrile) to afford tert-butyl (2R,3S,4S)-4-[(tert-butoxycarbonyl)oxy]-3-({[(3S)-1-(tert-butoxycarbonyl)pyrrolidin-3-yl]carbamoyl}oxy)-2-[(4-methoxyphenyl)methyl]pyrrolidine-1-carboxylate (80 mg, 92.6%) as a white solid. MS: m/z: Calc'd for C₃₂H₄₉N₃O₁₀ [M+Na]⁺658. found 658.

Step 2: To a solution of tert-butyl (2R,3S,4S)-4-[(tert-butoxycarbonyl)oxy]-3-({[(3S)-1-(tert-butoxycarbonyl)pyrrolidin-3-yl]carbamoyl}oxy)-2-[(4-methoxyphenyl)methyl]pyrrolidine-1-carboxylate (75 mg, 0.12 mmol) in DCM (5 mL) was added TFA (1 mL), the mixture was stirred at RT for 1 h. LCMS showed the starting material was consumed completely. The resulting solution was diluted with 10 mL of DCM and directly concentrated in vacuo to obtain the crude, which was purified by reversed-phase column chromatography (0.05% TFA and MeCN) and further purified by Prep-HPLC to obtain (2R,3S,4S)-4-hydroxy-2-[(4-methoxyphenyl) methyl]pyrrolidin-3-yl N-[(3S)-pyrrolidin-3-yl]carbamate; trifluoroacetic acid (20.5 mg, 38.1%) as a white solid. MS: m/z: Calc'd for C₁₇H₂₅N₃O₄ [M+H]⁺336. found 336. ¹H NMR (400 MHz, Methanol-d₄) δ 7.24 (d, J=8.2 Hz, 2H), 6.97-6.90 (m, 2H), 5.00-4.95 (m, 1H), 4.45-4.27 (m, 2H), 4.25-4.11 (m, 1H), 3.80-3.78 (m, 3H), 3.63-3.45 (m, 3H), 3.41-3.35 (m, 1H), 3.31 (d, J=8.6 Hz, 1H), 3.22 (d, J=12.6 Hz, 1H), 3.11 (dd, J=14.3, 6.9 Hz, 1H), 2.99 (dd, J=14.2, 8.7 Hz, 1H), 2.42-2.29 (m, 1H), 2.15-2.02 (m, 1H). Prep-HPLC conditions: Column: Atlantis T3 Prep OBD Column, 19*250 mm, 5 μm; Mobile Phase A: Water (0.05% TFA), Mobile Phase B: ACN; Flow rate: 25 mL/min mL/min; Gradient: 3% B to 25% B in 8 min; Wave Length: 254 nm/220 nm; RT1(min): 7.15

### Synthetic Procedure of V187692

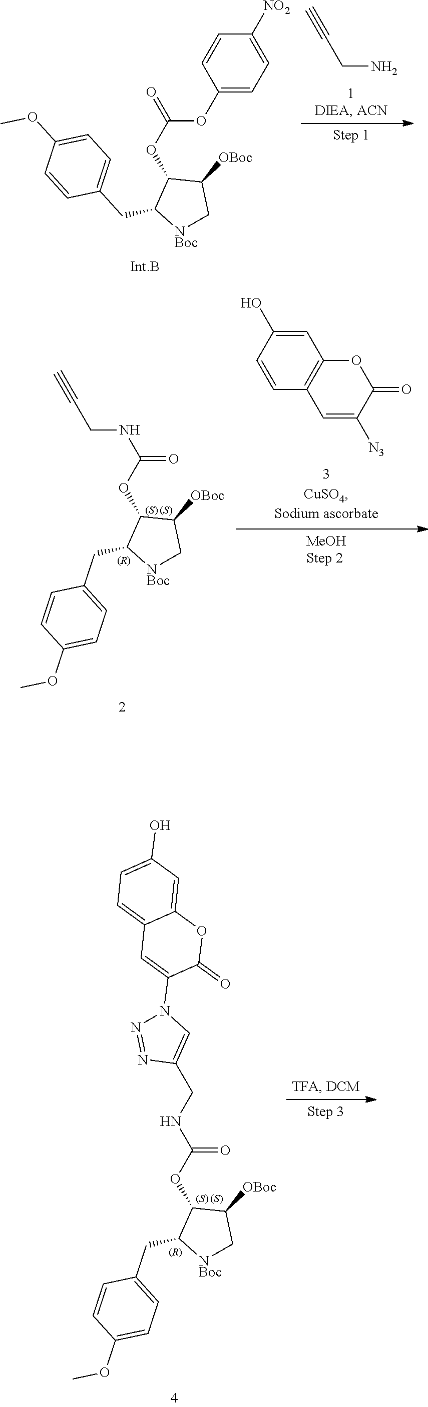

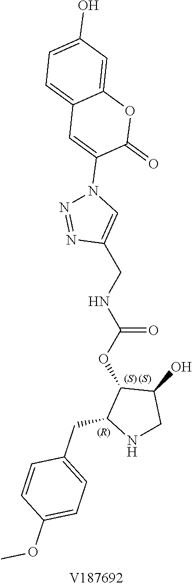

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 300 mg, 0.51 mmol, 1.0 eq.) and 2-propynylamine (56.15 mg, 1.02 mmol, 2.0 eq.) in MeCN (3 mL) was added DIEA (197.6 mg, 1.53 mmol, 3.0 eq.). The resulting reaction mixture was stirred at ambient temperature for overnight. After completion of the reaction monitored by LCMS, the mixture was purified directly by reversed-phase column chromatography (10 mmol/L NH₄HCO₃ in water and acetonitrile) to afford tert-butyl (2R,3S,4S)-4-[(tert-butoxycarbonyl)oxy]-2-[(4-methoxyphenyl)methyl]-3-{[(prop-2-yn-1-yl)carbamoyl]oxy}pyrrolidine-1-carboxylate (250 mg, 97.2%) as a light yellow oil. MS: m/z: Calc'd for C₂₆H₃₆N₂O₈ [M+H−56−100]*349. Found, 349.

Step 2: To a stirred solution of tert-butyl (2R,3S,4S)-4-[(tert-butoxycarbonyl)oxy]-2-[(4-methoxyphenyl)methyl]-3-{[(prop-2-yn-1-yl)carbamoyl]oxy}pyrrolidine-1-carboxylate (100 mg, 0.20 mmol, 1.0 eq.) and 3-azido-7-hydroxychromen-2-one (40.3 mg, 0.20 mmol, 1.0 eq.) in MeOH (3 mL) were added CuSO₄·5H₂O (49.5 mg, 0.20 mmol, 1.0 eq.) and sodium ascorbate (78.9 mg, 0.40 mmol, 2.0 eq.) at room temperature. The resulting reaction mixture was stirred at ambient temperature for overnight. The resulting mixture was filtered, the filter cake was washed with MeOH (1×1 mL). The filtrate was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 220 nm. This resulted in tert-butyl (2R,3S,4S)-4-[(tert-butoxycarbonyl)oxy]-3-[({[1-(7-hydroxy-2-oxochromen-3-yl)-1,2,3-triazol-4-yl]methyl}carbamoyl)oxy]-2-[(4-methoxyphenyl) methyl]pyrrolidine-1-carboxylate (120 mg, 85.6%) as a light yellow oil. MS: m/z: Calc'd for C₃₅H₄₁N₅O₁₁ [M+H]⁺708. Found, 708.

Step 3: To a stirred solution of tert-butyl (2R,3S,4S)-4-[(tert-butoxycarbonyl)oxy]-3-[({[1-(7-hydroxy-2-oxochromen-3-yl)-1,2,3-triazol-4-yl]methyl}carbamoyl)oxy]-2-[(4-methoxyphenyl) methyl]pyrrolidine-1-carboxylate (100 mg, 0.14 mmol, 1.0 eq.) in DCM (2 ml) was added TFA (1 mL) at 0° C. under nitrogen atmosphere. The resulting mixture was stirred at room temperature for 1 h. After completion of the reaction monitored by LCMS, the mixture was concentrated. The residue was purified by Prep-HPLC to afford (2R,3S,4S)-4-hydroxy-2-[(4-methoxyphenyl) methyl]pyrrolidin-3-yl N-{[1-(7-hydroxy-2-oxochromen-3-yl)-1,2,3-triazol-4-yl]methyl}carbamate; trifluoroacetic acid (28.7 mg, 32.6%) as a white solid. MS: m/z: Calc'd for C₂₅H₂₅N₅O₇ [M+H]⁺508. Found, 508. ¹H NMR (400 MHz, Methanol-d₄) δ 8.52 (s, 2H), 7.67 (d, J=8.6 Hz, 1H), 7.20 (d, J=8.3 Hz, 2H), 6.96-6.82 (m, 4H), 4.96 (d, J=4.1 Hz, 1H), 4.61-4.49 (m, 2H), 4.42 (d, J=4.2 Hz, 1H), 4.18-4.14 (m, 1H), 3.76 (s, 3H), 3.59 (dd, J=12.6, 4.2 Hz, 1H), 3.23 (d, J=12.6 Hz, 1H), 3.11 (dd, J=14.1, 7.5 Hz, 1H), 2.96 (dd, J=14.1, 8.3 Hz, 1H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 8% B to 31% B in 10 min; Wave Length: 254 nm/220 nm; RT1(min): 8.68

### Synthetic Procedure of V187698

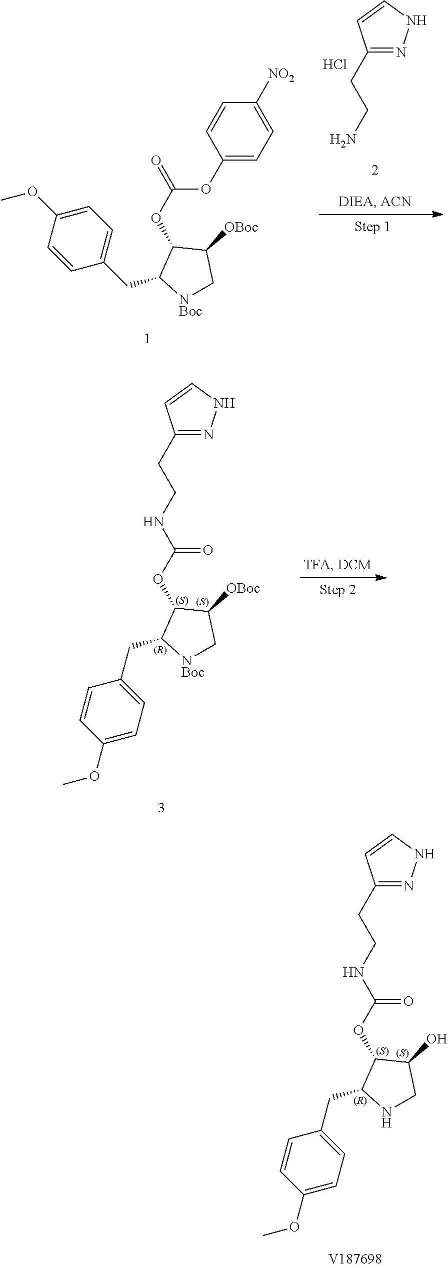

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (80 mg, 0.14 mmol, 1 eq) and betazole hydrochloride (20 mg, 0.14 mmol, 1 eq) in ACN (5 mL) was added DIEA (53 mg, 0.41 mmol, 3 eq). The resulting mixture was stirred at room temperature for 6 h. The mixture was concentrated and the residue was purified by reversed-phase flash chromatography to afford tert-butyl (2R,3S,4S)-4-[(tert-butoxycarbonyl)oxy]-2-[(4-methoxy phenyl)methyl]-3-({[2-(1H-pyrazol-3-yl)ethyl]carbamoyl}oxy)pyrrolidine-1-carboxylate (81 mg, 96.3%) as a colorless solid. MS: m/z: Calc'd for C₂₈H₄₀N₄O₈ [M+H]⁺561. Found, 561.

Step 2: To a stirred solution of tert-butyl (2R,3S,4S)-4-[(tert-butoxycarbonyl)oxy]-2-[(4-methoxyphenyl)methyl]-3-({[2-(1H-pyrazol-3-yl)ethyl]carbamoyl}oxy)pyrrolidine-1-carboxylate (78 mg, 0.14 mmol, 1 eq) in DCM (5 mL) was added TFA (1 mL) at 0° C. under nitrogen atmosphere. The resulting mixture was stirred at room temperature for 2 h. The mixture was concentrated and the crude product (55 mg) was purified by Prep-HPLC to afford (2R,3S,4S)-4-hydroxy-2-[(4-methoxyphenyl)methyl]pyrrolidin-3-yl N-[2-(1H-pyrazol-3-yl)ethyl]carbamate; trifluoroacetic acid (30.6 mg, 46.1%) as a light yellow semi-solid. MS: m/z: Calc'd for C₁₈H₂₄N₄O₄ [M+H]⁺361. Found, 361. ¹H NMR (400 MHz, Methanol-d₄) δ 7.59 (d, J=2.2 Hz, 1H), 7.24-7.16 (m, 2H), 6.96-6.88 (m, 2H), 6.25 (d, J=2.2 Hz, 1H), 4.92 (d, J=3.0 Hz, 1H), 4.36 (d, J=4.3 Hz, 1H), 4.18-4.09 (m, 1H), 3.79 (s, 3H), 3.59-3.53 (m, 1H), 3.52-3.43 (m, 2H), 3.20 (d, J=12.6 Hz, 1H), 3.08 (dd, J=14.1, 7.2 Hz, 1H), 2.99-2.87 (m, 3H). Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 24% B in 10 min; Wave Length: 254 nm/220 nm; RT1(min): 8.68

### Synthetic Procedure of V187700

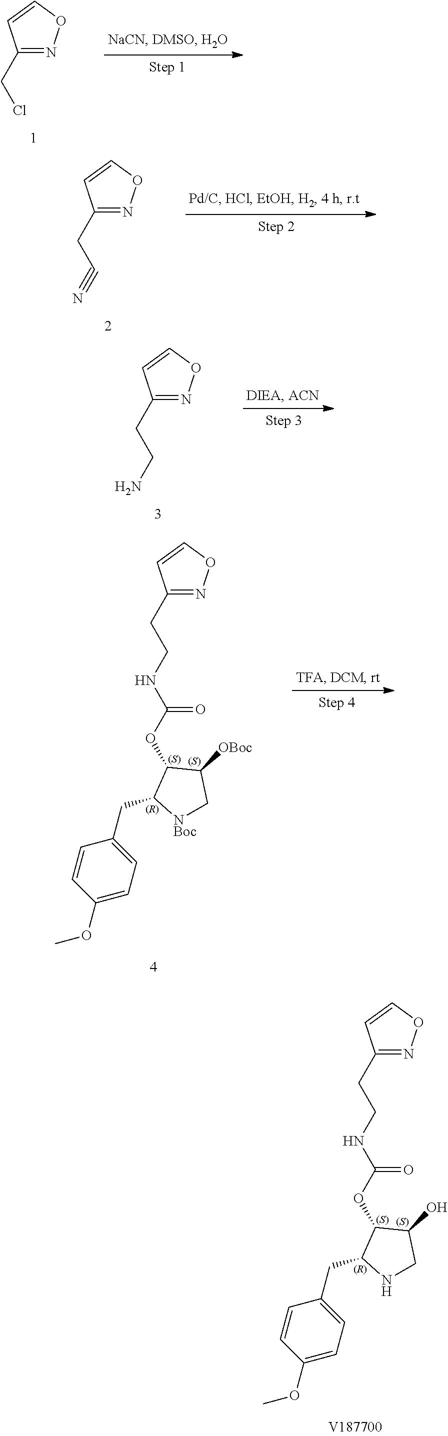

Step 1: To a stirred solution of 3-(chloromethyl)-1,2-oxazole (750 mg, 6.38 mmol, 1 eq.) in DMSO (7 mL) and H₂O (3 mL) was added NaCN (469.1 mg, 9.57 mmol, 1.5 eq.) at 0° C. The resulting mixture was stirred at room temperature for overnight. The reaction mixture was quenched with water and extracted with EA. The extracts were washed with brine, dried over anhydrous sodium sulfate, filtered and concentrated. The residue was purified by Prep-TLC PE/EA (3:1) to afford 2-(1,2-oxazol-3-yl)acetonitrile (106 mg, 15.3%) as a yellow oil. ¹H NMR (400 MHz, Chloroform-d) δ 8.60-8.40 (m, 1H), 6.50 (d, J=1.7 Hz, 1H), 3.89 (s, 2H).

Step 2: Under a nitrogen atmosphere, Pd/C (104.3 mg, 0.98 mmol) was added to a solution of tert-butyl 2-(1,2-oxazol-3-yl)acetonitrile (106 mg, 0.98 mmol, 1 eq.) and HCl (0.36 mg, 0.01 mmol, 0.01 eq.) in Ethanol (5 mL), H₂ was subsequently introduced into the reaction system, and the resulting mixture was stirred at r.t. for 2 h. The reaction mixture was filtered to obtain 2-(1,2-oxazol-3-yl)ethanamine (100 mg, 90.9%) as a yellow oil. MS: m/z: Calc'd for C₅H₈N₂O [M+H]⁺113. found 113.

Step 3: To a stirred solution of 2-(1,2-oxazol-3-yl) ethanamine (45.7 mg, 0.40 mmol, 2 eq.) and tert-butyl(2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (572.1 mg, 0.97 mmol, 1 eq.) in ACN (4 mL) was added DIEA (79.0 mg, 0.61 mmol, 3 eq.) at 0° C. The resulting mixture was stirred at room temperature for overnight. After completion of the reaction monitored by LCMS. The resulting mixture was concentrated under reduced pressure. The residue was purified by reversed-phase flash to afford tert-butyl (2R,3S,4S)-4-[(tert-butoxycarbonyl)oxy]-2-[(4-methoxyphenyl)methyl]-3-({[2-(1,2-oxazol-3-yl)ethyl]carbamoyl}oxy)pyrrolidine-1-carboxylate (100 mg) as a yellow oil. MS: m/z: Calc'd for C₂₈H₃₉N₃O₉ [M+22]⁺ 584. found 584.

Step 4: To a stirred mixture of tert-butyl (2R,3S,4S)-4-[(tert-butoxycarbonyl)oxy]-2-[(4-methoxyphenyl)methyl]-3-({[2-(1,2-oxazol-3-yl)ethyl]carbamoyl}oxy)pyrrolidine-1-carboxylate (100 mg, 0.17 mmol, 1 eq.) in DCM (5 mL) was added TFA (1 mL) at 0° C. The resulting mixture was stirred at room temperature for 2 h. After completion of the reaction monitored by LCMS. The resulting mixture was concentrated under reduced pressure. The crude product (50 mg) was purified by Prep-HPLC to afford (2R,3S,4S)-4-hydroxy-2-[(4-methoxyphenyl)methyl]pyrrolidin-3-yl N-[2-(1,2-oxazol-3-yl)ethyl]carbamate; trifluoroacetic acid (37.1 mg, 43.2%) as a yellow solid. MS: m/z: Calc'd for C₁₈H₂₃N₃O₅ [M+H]⁺362. found 362. ¹H NMR (400 MHz, Methanol-d₄) δ 8.59 (d, J=1.7 Hz, 1H), 7.25-7.17 (m, 2H), 6.95-6.88 (m, 2H), 6.47 (d, J=1.7 Hz, 1H), 4.89 (s, 1H), 4.38-4.32 (m, 1H), 4.13-4.02 (m, 1H), 3.79 (s, 3H), 3.65-3.43 (m, 3H), 3.20 (d, J=12.6 Hz, 1H), 3.07 (dd, J=13.9, 7.6 Hz, 1H), 2.96 (t, J=6.7 Hz, 3H). Column: Xselect CSH Prep C18 C18 Column, 30*150 mm, 5 μm; Mobile Phase A: Water (0.5% TFA), Mobile Phase B: ACN; Flow rate: 60 mL/min mL/min; Gradient: 2% B to 27% B in 10 min; Wave Length: 254 nm/220 nm; RT1(min): 8.68

### Synthetic Procedure of V187723

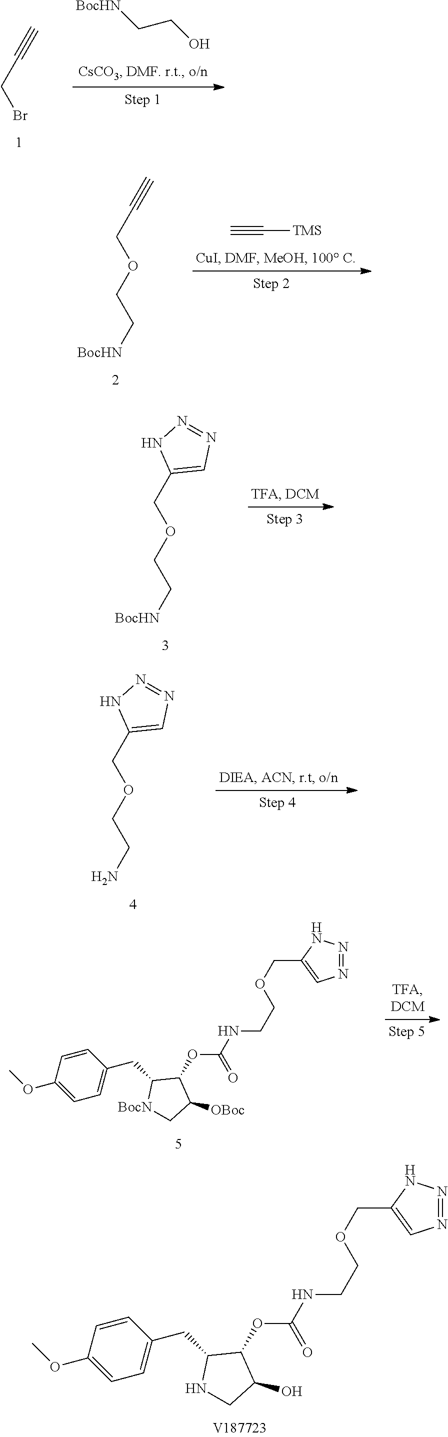

Step 1: To a stirred solution of propargyl bromide (1.1 g, 9.24 mmol, 1 eq.) and tert-butyl N-(2-hydroxyethyl)carbamate (1.49 g, 9.24 mmol, 1 eq.) in DMF (10 mL) was added Cs₂CO₃ (9.0 g, 27.74 mmol, 3 eq.) at 0° C. The resulting mixture was stirred at room temperature for overnight. The reaction mixture was quenched with water and extracted with EA. The extracts were washed with brine, dried over anhydrous sodium sulfate, filtered and concentrated. The residue was purified by silica gel column chromatography, eluted with PE/EA (10:1) to afford tert-butyl N-[2-(prop-2-yn-1-yloxy)ethyl]carbamate (1.5 g, 81.2%) as a yellow oil. ¹H NMR (400 MHz, DMSO-d₆) δ 6.85-6.77 (m, 1H), 4.12 (d, J=2.4 Hz, 2H), 3.45-3.38 (m, 3H), 3.07 (q, J=6.0 Hz, 2H), 1.37 (s, 9H)

Step 2: To a stirred solution of tert-butyl N-[2-(prop-2-yn-1-yloxy)ethyl]carbamate (440 mg, 2.20 mmol, 1 eq.) and trimethylsilylacetylene (433.7 mg, 4.41 mmol, 2 eq.) in DMF (5 mL) and MeOH (5 mL) was added CuI (420.7 mg, 2.20 mmol, 1 eq.) at room temperature. The resulting mixture was stirred at 100° C. for overnight. The resulting mixture was filtered, the filter cake was washed with MeOH (2×10 mL). The filtrate was concentrated under reduced pressure. The resulting mixture was concentrated under reduced pressure. The residue was purified by reversed-phase flash to obtain tert-butyl N-[2-(3H-1,2,3-triazol-4-ylmethoxy)ethyl]carbamate (500 mg, 93.4%) as a yellow oil. MS: m/z: Calc'd for C₁₀H₁₈N₄O₃[M+H]⁺243. found 243.

Step 3: To a stirred solution of tert-butyl N-[2-(3H-1,2,3-triazol-4-ylmethoxy)ethyl]carbamate (300 mg, 1.23 mmol, 1 eq.) in DCM (5 mL) was added TFA (1 mL) at 0° C. The resulting mixture was stirred at room temperature for 3 h. After completion of the reaction monitored by LCMS. The resulting mixture was concentrated under vacuum to afford 2-(3H-1,2,3-triazol-4-ylmethoxy)ethanamine (160 mg, 90.8%) as a yellow oil. MS: m/z: Calc'd for C₅H₁₀N₄O [M+H]⁺ 143; found 143.

Step 4: To a stirred solution of 2-(3H-1,2,3-triazol-4-ylmethoxy)ethanamine (48.3 mg, 0.340 mmol, 2 eq.) and tert-butyl (2R,3S,4S)-4-[(tert-butoxycarbonyl)oxy]-2-[(4-methoxyphenyl) methyl]-3-[(4-nitrophenoxycarbonyl)oxy]pyrrolidine-1-carboxylate (100 mg, 0.17 mmol, 1.00 eq.) in ACN (5 mL) was added DIEA (65.8 mg, 0.51 mmol, 3 eq.) at 0° C. The resulting mixture was stirred at room temperature for overnight. After completion of the reaction monitored by LCMS. The resulting mixture was concentrated under vacuum. The residue was purified by reversed-phase flash to obtain tert-butyl (2R,3S,4S)-4-[(tert-butoxycarbonyl)oxy]-2-[(4-methoxyphenyl)methyl]-3-({[2-(3H-1,2,3-triazol-4-ylmethoxy)ethyl]carbamoyl}oxy) pyrrolidine-1-carboxylate (100 mg, 99.4%) as a yellow oil. MS: m/z: Calc'd for C₂₈H₄₁N₅O₉ [M-100]⁺ 492. found 492.

Step 5: To a stirred solution of tert-butyl (2R,3S,4S)-4-[(tert-butoxycarbonyl)oxy]-2-[(4-methoxyphenyl)methyl]-3-({[2-(3H-1,2,3-triazol-4-ylmethoxy)ethyl]carbamoyl}oxy) pyrrolidine-1-carboxylate (80 mg, 0.13 mmol, 1 eq.) in DCM (5 mL) was added TFA (1 mL) at 0° C. The resulting mixture was stirred at room temperature for 2 h. After completion of the reaction monitored by LCMS. The resulting mixture was concentrated under reduced pressure. The crude product (50 mg) was purified by Prep-HPLC to afford 4-[hydroxy({[2-(1,2,3-triazolidin-4-ylmethoxy)ethyl]amino})methoxy]-5-[(4-methoxycyclohexyl)methyl]pyrrolidin-3-ol; trifluoroacetic acid (23.3 mg, 32.5%) as a yellow solid. MS: m/z: Calc'd for C₁₈H₂₅N₅O₅ [M+H]⁺392. found 392. ¹H NMR (400 MHz, Methanol-d4) δ 7.83 (s, 1H), 7.21 (d, J=8.5 Hz, 2H), 6.90 (d, J=8.4 Hz, 2H), 4.92 (d, J=3.4 Hz, 1H), 4.70 (s, 2H), 4.37 (d, J=4.2 Hz, 1H), 4.14-4.02 (m, 1H), 3.79 (s, 3H), 3.67-3.53 (m, 3H), 3.38-3.35 (m, 2H), 3.20 (d, J=12.7 Hz, 1H), 3.10 (dd, J=14.1, 7.3 Hz, 1H), 2.95 (dd, J=14.2, 8.5 Hz, 1H). Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 23% B in 10 min; Wave Length: 254 nm/220 nm; RT1(min): 8.68

### Synthetic Procedure of V187730

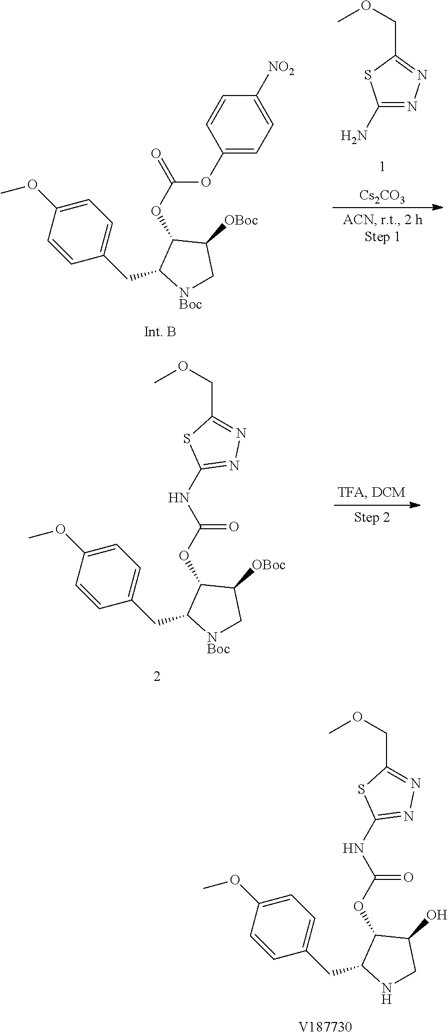

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int.B, 80 mg, 0.01 mmol, 1 eq.) in ACN (5 mL) were added Cs₂CO₃ (88.5 mg, 0.27 mmol, 2 eq.) and 5-(methoxymethyl)-1,3,4-thiadiazol-2-amine (39.4 mg, 0.27 mmol, 2 eq.) at r.t. The reaction was stirred at r.t. for 12 h. The reaction mixture was quenched with water and extracted with EA. The extracts were washed with brine, dried over anhydrous sodium sulfate, filtered and concentrated. The residue was purified by flash to obtained tert-butyl (2R,3S,4S)-4-[(tert-butoxycarbonyl) oxy]-3-({[5-(methoxymethyl)-1,3,4-thiadiazol-2-yl]carbamoyl}oxy)-2-[(4-methoxyphenyl) methyl]pyrrolidine-1-carboxylate (75 mg, 92.7%) as a white solid. MS: m/z: Calc'd for C₂₇H₃₈N₄O₉S [M+H]⁺595. Found, 595.

Step 2: To a stirred solution of tert-butyl (2R,3S,4S)-4-[(tert-butoxycarbonyl)oxy]-3-({[5-(methoxymethyl)-1,3,4-thiadiazol-2-yl]carbamoyl}oxy)-2-[(4-methoxyphenyl)methyl]pyrrolidine-1-carboxylate (75 mg, 0.12 mmol, 1 eq.) in DCM (3 mL) were added TFA (1 mL) at 0° C. The reaction was stirred at r.t. for 2 h. The resulting mixture was concentrated under reduced pressure. The crude product (70 mg) was purified by Prep-HPLC to afford (2R,3S,4S)-4-hydroxy-2-[(4-methoxyphenyl)methyl]pyrrolidin-3-yl N-[5-(methoxymethyl)-1,3,4-thiadiazol-2-yl]carbamate (23.3 mg, 46.7%) as a white solid. MS: m/z: Calc'd for C₁₇H₂₂N₄O₅S[M+H]⁺395. Found, 395. ¹H NMR (400 MHz, Methanol-d₄) δ 7.30-7.22 (m, 2H), 6.96-6.88 (m, 2H), 5.18-5.12 (m, 1H), 4.78 (s, 2H), 4.56-4.50 (m, 1H), 4.30-4.25 (m, 1H), 3.79 (s, 3H), 3.64-3.61 (m, 1H), 3.48 (s, 3H), 3.29 (d, J=12.7 Hz, 1H), 3.18-3.13 (m, 1H), 3.05-3.01 (m, 1H).

Prep-HPLC-conditions: Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 2% B to 31% B in 10 min; Wave Length: 254 nm/220 nm; RT1(min): 8.68

### Synthetic Procedure of V187739

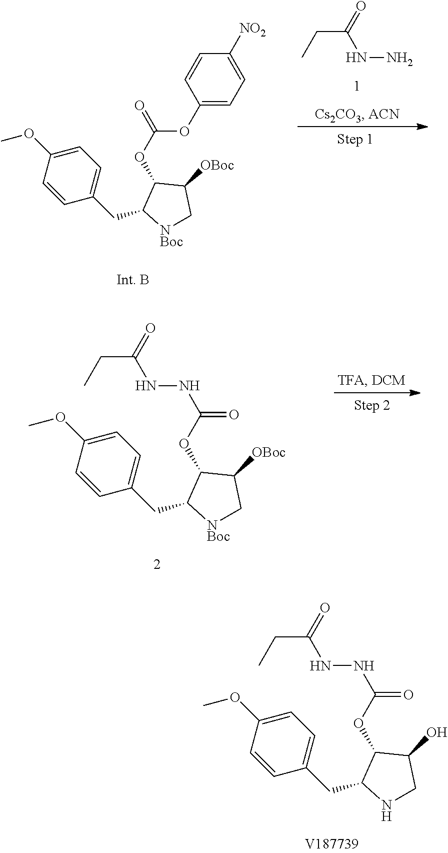

Step 1: To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (500 mg, 0.85 mmol, 1 eq) and propionic acid hydrazide (75 mg, 0.85 mmol, 1 eq) in ACN (15 mL) was added Cs₂CO₃ (830 mg, 2.55 mmol, 3 eq). The resulting mixture was stirred at 80° C. for 4 h under nitrogen atmosphere. The mixture was filtered and the filtrate was concentrated. The residue was purified by reversed-phase flash chromatography to afford tert-butyl (2R,3S,4S)-4-[(tert-butoxycarbonyl)oxy]-2-[(4-methoxyphenyl)methyl]-3-[(N′-propanoylhydrazinecarbonyl) oxy]pyrrolidine-1-carboxylate (370 mg, 81.0%) as a light yellow solid. MS: m/z: Calc'd for C₂₆H₃₉N₃O₉ [M+H−100−56]⁺382. Found, 382.

Step 2: To a stirred solution of tert-butyl (2R,3S,4S)-4-[(tert-butoxycarbonyl)oxy]-2-[(4-methoxyphenyl)methyl]-3-[(N′-propanoylhydrazinecarbonyl)oxy]pyrrolidine-1-carboxylate (55 mg, 0.10 mmol, 1 eq) in DCM (5 mL) was added TFA (1 mL) dropwise at 0° C. under nitrogen atmosphere. The resulting mixture was stirred at room temperature for 2 h. The mixture was concentrated and the crude product (45 mg) was purified by Prep-HPLC to afford 1-{[(2R,3S,4S)-4-hydroxy-2-[(4-methoxyphenyl)methyl]pyrrolidin-3-yl]oxy}-N′-propanoylformohydrazide; trifluoroacetic acid (21.8 mg, 46.9%) as a purple solid. MS: m/z: Calc'd for C₁₆H₂₃N₃O₅ [M+H]⁺338. Found, 338. ¹H NMR (400 MHz, Methanol-d₄) δ 7.27 (d, J=8.5 Hz, 2H), 6.98-6.90 (m, 2H), 4.98 (d, J=3.3 Hz, 1H), 4.45 (s, 1H), 4.18 (s, 1H), 3.80 (s, 3H), 3.61 (d, J=12.0 Hz, 1H), 3.24 (d, J=12.6 Hz, 1H), 3.20-3.10 (m, 1H), 3.04-2.94 (m, 1H), 2.35-2.24 (m, 2H), 1.19 (t, J=7.6 Hz, 3H). Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 17% B in 10 min; Wave Length: 254 nm/220 nm; RT1(min): 8.68

### Synthetic Procedure of V187741

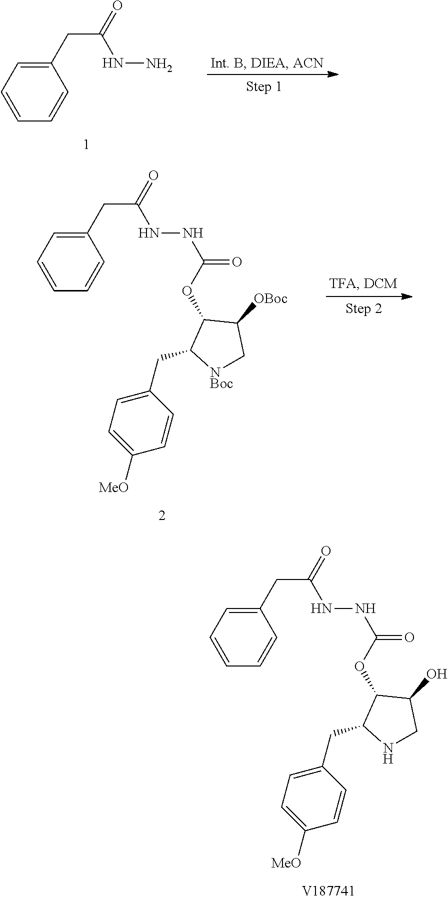

Step 1: To the solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (500 mg, 0.85 mmol, 1 equiv) and 2-phenylacetohydrazide (153 mg, 1.02 mmol, 1.2 equiv) in ACN (10 mL) was added TEA (172 mg, 1.70 mmol, 2 equiv). The mixture was stirred at rt for overnight. LCMS showed the reaction was OK. The mixture was concentrated to give the crude product which was purified by reverse phase flash. The tert-butyl (2R,3S,4S)-4-[(tert-butoxy carbonyl)oxy]-2-[(4-methoxyphenyl)methyl]-3-{[(2-phenylacetohydrazido)carbonyl]oxy}pyrrolidine-1-carboxylate (360 mg, 70.7%) was obtained as a white solid. MS: m/z: Calc'd for C₃₁H₄₁N₃O₉ [M−H]598. found 598.

Step 2: To a solution of tert-butyl (2R,3S,4S)-4-[(tert-butoxycarbonyl)oxy]-2-[(4-methoxy phenyl)methyl]-3-{[(2-phenylacetohydrazido)carbonyl]oxy}pyrrolidine-1-carboxylate (55 mg, 0.092 mmol, 1 equiv) in DCM (2 mL) was added TFA (1 mL) slowly. The mixture was stirred at rt for 1 hour. LCMS showed the reaction was completed. The mixture was concentrated to give the crude product, which was purified by Prep HPLC. The N′-({[(2R,3S,4S)-4-hydroxy-2-[(4-methoxyphenyl)methyl]pyrrolidin-3-yl]oxy}carbonyl)-2-phenylacetohydrazide; trifluoroacetic acid (20.7 mg, 43.8%) was obtained as a white solid. MS: m/z: Calc'd for C₂₁H₂₅N₃O₅ [M+H]⁺400. found 400. ¹H NMR (400 MHz, Methanol-d₄) δ 7.40-7.27 (m, 4H), 7.31-7.23 (m, 3H), 6.96-6.89 (m, 2H), 4.97 (d, J=3.1 Hz, 1H), 4.44 (s, 1H), 4.22-4.13 (m, 1H), 3.79 (s, 3H), 3.61 (s, 3H), 3.23 (d, J=12.5 Hz, 1H), 3.18-3.09 (m, 1H), 3.02-2.97 (m, 1H).

Prep-HPLC-conditions: Column: Xselect CSH Prep C18 C18 Column, 30*150 mm, 5 μm; Mobile Phase A: Water (0.1% FA), Mobile Phase B: ACN; Flow rate: 60 mL/min mL/min; Gradient: 4% B to 34% B in 10 min; Wave Length: 254 nm/220 nm; RT1(min): 8.68.

### Synthetic Procedure of V187753

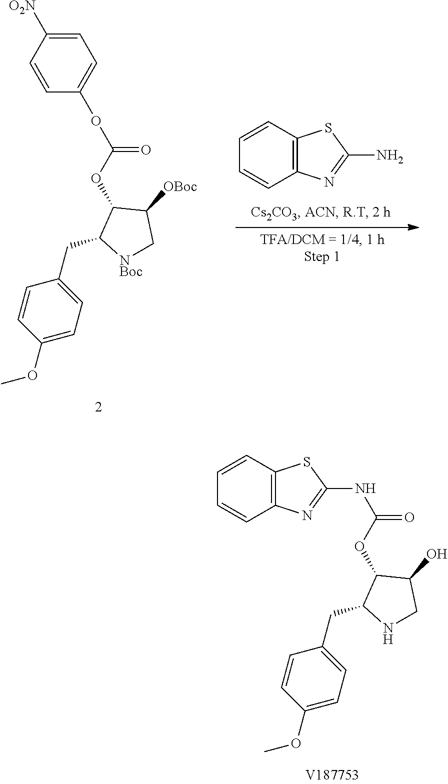

To a stirred mixture of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (80 mg, 0.14 mmol, 1 eq.) and 2-aminobenzothiazole (20.41 mg, 0.14 mmol, 1 eq.) in ACN (10 mL) was added Cs₂CO₃ (132.85 mg, 0.41 mmol, 3 eq.) at room temperature. The resulting mixture was stirred for 2 h and the mixture was concentrated under vacuum. To the residue was added DCM (6 mL) and TFA (2 mL) at room temperature. The resulting mixture was stirred at r.t. for 1 h. The mixture was concentrated under vacuum. The residue was purified by Prep-HPLC with the following conditions (Column: Xselect CSH Prep C18 C18 Column, 30*150 mm, 5 μm; Mobile Phase A: Water (0.05% TFA), Mobile Phase B: ACN; Flow rate: 60 mL/min mL/min; Gradient: 17% B to 38% B in 10 min; Wave Length: 254 nm/220 nm; RT1(min): 8.68) to afford (2R,3S,4S)-4-hydroxy-2-[(4-methoxyphenyl)methyl]pyrrolidin-3-yl N-(1,3-benzothiazol-2-yl)carbamate; trifluoroacetic acid (17.5 mg, 24.67%) as a white solid. MS: m/z: Calc'd for C₂₀H₂₁N₃O₄S [M+H]⁺400. found [M+H]⁺400. ¹H NMR (400 MHz, Methanol-d₄) δ 7.86 (dd, J=8.0, 1.2 Hz, 1H), 7.73 (d, J=7.9 Hz, 1H), 7.45-7.41 (m, 1H), 7.36-7.26 (m, 1H), 7.29-7.22 (m, 2H), 6.95-6.86 (m, 2H), 5.15 (d, J=3.5 Hz, 1H), 4.56-4.50 (m, 1H), 4.29-4.20 (m, 1H), 3.77 (s, 3H), 3.66 (dd, J=12.7, 4.3 Hz, 1H), 3.29 (d, J=12.7 Hz, 1H), 3.19 (dd, J=14.2, 7.2 Hz, 1H), 3.07 (dd, J=14.2, 8.6 Hz, 1H).

### Synthetic Procedure of V0074

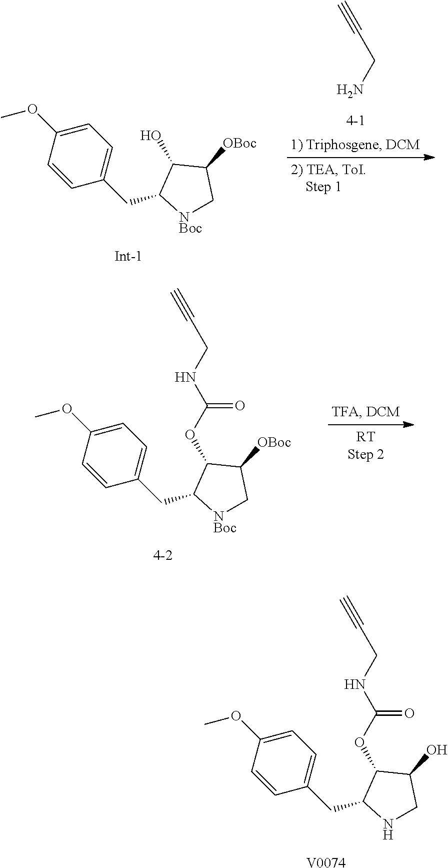

Step 1: To a stirred solution of 2-propynylamine (130 mg, 2.36 mmol, 5 eq.) in DCM (2 mL) was added Triphosgene (56.1 mg, 0.19 mmol, 0.4 eq.) and TEA (238.9 mg, 2.36 mmol, 5 eq.) at 0° C. and the resulting mixture was stirred at room temperature for 30 min. Then tert-butyl (2R,3S,4S)-4-[(tert-butoxycarbonyl)oxy]-3-hydroxy-2-[(4-methoxyphenyl)methyl]pyrrolidine-1-carboxylate (200 mg, 0.47 mmol, 1 eq.), DMAP (115.4 mg, 0.94 mmol, 2 eq.) and Toluene (5 mL) were added at room temperature. The resulting mixture was stirred at 80° C. for 12 h. Upon completion, the reaction mixture was quenched with water and extracted with EA. The organic phase was washed with brine, dried over anhydrous sodium sulfate, filtered and concentrated. The residue was purified by Prep-TLC to obtain tert-butyl (2R,3S,4S)-4-((tert-butoxycarbonyl)oxy)-2-(4-methoxybenzyl)-3-((prop-2-yn-1-ylcarbamoyl)oxy)pyrrolidine-1-carboxylate (80 mg, 51.42%) as a light-yellow oil. MS: m/z: Calc'd for C₂₆H₃₆N₂O₈ [M+H]⁺504. found 504.

Step 2: The compound was synthesized according to Boc Deprotection; General Procedure III using tert-butyl (2R,3S,4S)-4-((tert-butoxycarbonyl)oxy)-2-(4-methoxybenzyl)-3-((prop-2-yn-1-ylcarbamoyl)oxy)pyrrolidine-1-carboxylate (4-2, 80 mg, 0.16 mmol). The crude product was purified by Prep-HPLC to afford a yellow solid; yield: 26.1 mg (38.6%). MS: m/z: Calc'd for C₁₆H₂₀N₂O₄ [M+H]⁺305. found 305. ¹H NMR (400 MHz, DMSO-d₆) δ 9.50 (s, 1H), 9.07 (s, 1H), 7.71-7.69 (m, 1H), 7.22 (d, J=8.4 Hz, 2H), 6.90 (d, J=8.0 Hz, 2H), 5.99 (s, 1H), 4.76-4.68 (m, 1H), 4.21-4.19 (m, 1H), 3.98 (s, 1H), 3.92-3.80 (m, 2H), 3.74 (s, 4H), 3.57-3.50 (m, 1H), 3.21 (s, 1H), 3.07-3.05 (m, 1H), 3.01-2.83 (m, 2H). Prep-HPLC purification conditions: Column: UV 254 nm/220 nm Xselect CSH Prep OBD C18 Column, 30*150 mm, 5 μm; Mobile Phase A: Water (0.1% TFA), Mobile Phase B: ACN 60 mL/min; Gradient: 2% B to 26% B in 10 min 6.43 Wave Length: 254 nm/220 nm nm; RT1(min): 6.2

### Synthetic Procedure of V187642

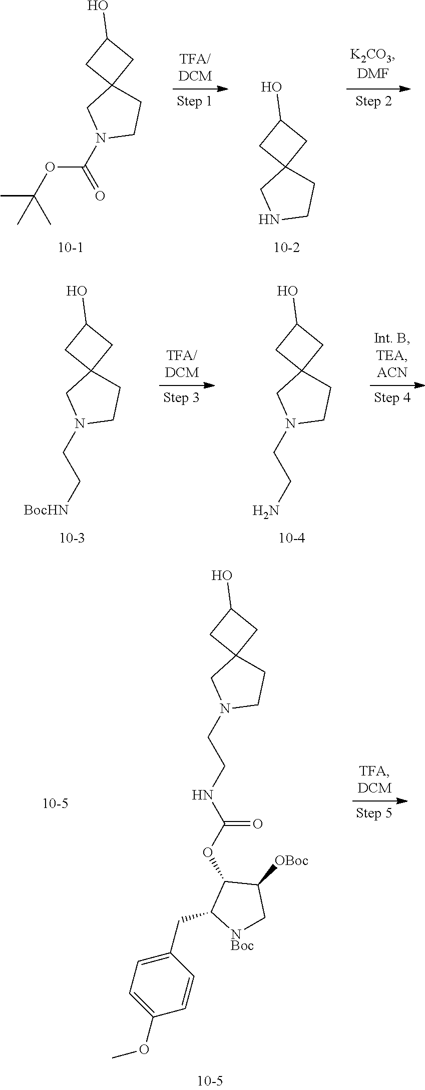

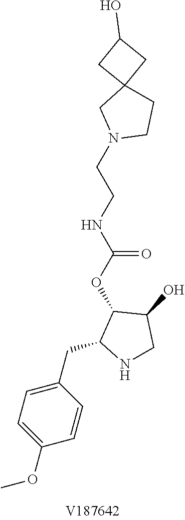 [SMILES: COc1ccc(C[C@H]2NC[C@H](O)[C@H]2OC(=O)NCCN2CCC3(CC(O)C3)C2)cc1]

Step 1: To a solution of tert-butyl 2-hydroxy-6-azaspiro[3.4]octane-6-carboxylate (10-1, 300 mg, 1.32 mmol, 1 eq.) in DCM (5 mL) was added TFA (1 mL). The mixture was stirred at room temperature for 1 hour. LCMS showed the reaction was complete. The mixture was concentrated to give the crude product which was used directly in the next step without further purification. MS: m/z: Calc'd for C₇H₁₃NO [M+H]⁺128. found 128.

Step 2: To a solution of 6-azaspiro[3.4]octan-2-ol (10-2, 160 mg, 1.26 mmol, 1 eq.) and tert-butyl N-(2-bromoethyl)carbamate (423 mg, 1.89 mmol, 1.5 eq.) in DMF (5 mL) was added K₂CO₃ (869 mg, 6.29 mmol, 5 eq.). The mixture was stirred at room temperature for overnight. The mixture was diluted with EA, washed with water, dried, filtered and concentrated to give the crude product which was used directly in the next step without further purification as a yellow oil. MS: m/z: Calc'd for C₁₄H₂₆N₂O₃ [M+H]⁺271. found 271.

Step 3: To a solution of tert-butyl N-(2-{2-hydroxy-6-azaspiro[3.4]octan-6-yl}ethyl)carbamate (10-3, 55 mg, 0.20 mmol, 1 eq.) in DCM (5 mL) was added TFA (1 mL). The mixture was stirred at room temperature for 1 hour. The mixture was concentrated to give the crude product which was used directly in the next step without further purification. MS: m/z: Calc'd for C₉H₁₈N₂O [M+H]⁺171. found 171.

Step 4: To a solution of tert-butyl (2R,3S,4S)-4-[(tert-butoxycarbonyl)oxy]-2-[(4-methoxyphenyl)methyl]-3-{[(4-nitrophenyl)carbamoyl]oxy}pyrrolidine-1-carboxylate (10-4, 100 mg, 0.17 mmol, 1 eq.) and 6-(2-aminoethyl)-6-azaspiro[3.4]octan-2-ol (29 mg, 0.17 mmol, 1 eq.) in ACN (5 mL) was added TEA (34 mg, 0.34 mmol, 2 eq.). The mixture was stirred at room temperature for 1 hour. LCMS showed the reaction was complete. The mixture was concentrated to give the crude product which was used directly in the next step without further purification. MS: m/z: Calc'd for C₃₂H₄₉N₃O₉ [M+H]⁺620. found 620.

Step 5: The compound was synthesized according to Boc Deprotection; General Procedure III using tert-butyl (2R,3S,4S)-4-[(tert-butoxycarbonyl)oxy]-3-{[(2-{2-hydroxy-6-azaspiro[3.4]octan-6-yl}ethyl)carbamoyl]oxy}-2-[(4-methoxyphenyl)methyl]pyrrolidine-1-carboxylate (10-5, 80 mg, 0.13 mmol). The crude product was purified by Prep-HPLC to afford a light yellow semi-solid; yield: 18.1 mg (26.1%). MS: m/z: Calc'd for C₂₂H₃₃N₃O₅ [M+H]⁺420. found 420. ¹H NMR (400 MHz, Methanol-d₄) δ 7.25 (d, J=8.3 Hz, 2H), 6.94 (d, J=8.1 Hz, 2H), 5.01 (d, J=3.4 Hz, 1H), 4.42 (d, J=4.2 Hz, 1H), 4.36-4.13 (m, 2H), 3.80 (s, 3H), 3.76-3.71 (m, 2H), 3.64-3.49 (m, 3H), 3.39-3.32 (m, 3H), 3.27-3.16 (m, 2H), 3.17-3.07 (m, 1H), 3.04-2.94 (m, 1H), 2.57-2.35 (m, 2H), 2.29-1.94 (m, 4H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 17% B in 10 min; Wave Length: 254 nm/220 nm; RT1(min): 8.68.

### Synthetic Procedure of V187754

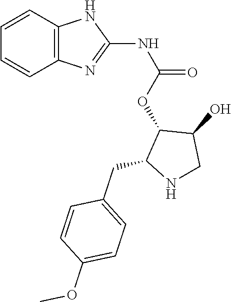 [SMILES: COc1ccc(C[C@H]2NC[C@H](O)[C@H]2OC(=O)Nc2nc3ccccc3[nH]2)cc1]

The title compound was prepared in 27.0% overall yield as a white solid according to Substitution reaction for synthesis of Carbamate; General Procedure II using 2-aminobenzimidazole in STEP 1; Boc Deprotection; General Procedure III in STEP 2. MS: m/z Calc'd for C₂₀H₂₂N₄O₄ [M+H]⁺383. Found, 383. ¹H NMR (400 MHz, Methanol-d₄) δ 9.62-9.60 (m, 2H), 7.50 (d, J=5.9 Hz, 2H), 7.35-7.17 (m, 4H), 6.95-6.85 (m, 2H), 6.05 (s, 1H), 4.96 (d, J=3.9 Hz, 1H), 4.39 (d, J=3.9 Hz, 1H), 4.07 (s, 1H), 3.71 (s, 3H), 3.47 (d, J=5.0 Hz, 2H), 3.11-2.97 (m, 3H). Prep-HPLC purification conditions: Column: Xselect CSH Prep C18 Column, 30*150 mm, 5 μm; Mobile Phase A: Water (0.05% HCl), Mobile Phase B: ACN; Flow rate: 60 mL/min; Gradient: 2% B to 25% B in 10 min; Wave Length: 254 nm/220 nm; RT1(min): 8.68.

### Synthetic Procedure of V187767

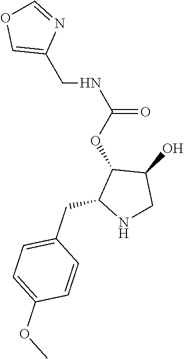 [SMILES: COc1ccc(C[C@H]2NC[C@H](O)[C@H]2OC(=O)NCc2cocn2)cc1]

The title compound was prepared in 25.8% overall yield as a white solid according to Substitution reaction for synthesis of Carbamate; General Procedure II using 1-(1,3-oxazol-4-yl)methanamine in STEP 1; Boc Deprotection; General Procedure III in STEP 2. MS: m/z Calc'd for C₁₇H₂₁N₃O₅ [M+H]⁺348. Found, 348. ¹H NMR (400 MHz, Methanol-d₄) δ 8.21 (s, 1H), 7.86 (s, 1H), 7.21 (d, J=8.3 Hz, 2H), 6.90 (d, J=8.3 Hz, 2H), 4.95 (d, J=3.5 Hz, 1H), 4.40 (d, J=4.2 Hz, 1H), 4.38-4.23 (m, 2H), 4.18-4.14 (m, 1H), 3.79 (s, 3H), 3.58 (dd, J=12.6, 4.3 Hz, 1H), 3.21 (d, J=12.6 Hz, 1H), 3.10 (dd, J=14.1, 7.3 Hz, 1H), 2.96 (dd, J=14.1, 8.4 Hz, 1H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.68.

### Synthetic Procedure of V187679

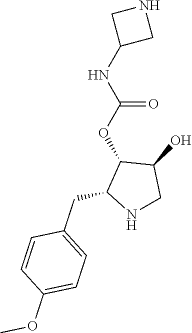 [SMILES: COc1ccc(C[C@H]2NC[C@H](O)[C@H]2OC(=O)NC2CNC2)cc1]

The title compound was prepared in 38.6% overall yield as yellow solid according to Substitution reaction for synthesis of Carbamate; General Procedure II using tert-butyl 3-aminoazetidine-1-carboxylate in STEP 1; Boc Deprotection; General Procedure III using ZnBr₂ instead of TFA in STEP 2. MS: m/z Calc'd for C₁₆H₂₃N₃O₄ [M+H]⁺322. found 322. ¹H NMR (400 MHz, Methanol-d₄) δ 8.58 (s, 2H), 7.23 (d, J=8.5 Hz, 2H), 6.96-6.89 (m, 2H), 4.94 (s, 1H), 4.64-4.54 (m, 1H), 4.39-4.16 (m, 5H), 4.10 (s, 1H), 3.79 (s, 3H), 3.56 (dd, J=12.6, 4.5 Hz, 1H), 3.16 (d, J=12.5 Hz, 1H), 3.12-2.93 (m, 2H). Prep-HPLC purification conditions: Column: Welch Ultimate XB-C18 50*250, 10 μm; Mobile Phase A: Water (0.1% FA), Mobile Phase B: ACN; Flow rate: 60 mL/min; Gradient: 0% B to 10% B in 20 min; Wave Length: 254 nm/220 nm; RT1(min): 15.35.

### Synthetic Procedure of V187686

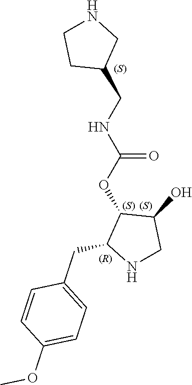

The title compound was prepared in 55.2% overall yield as yellow solid according to Substitution reaction for synthesis of Carbamate; General Procedure II using tert-butyl (R)-3-(aminomethyl)pyrrolidine-1-carboxylate in STEP 1; Boc Deprotection; General Procedure III using HCl (4 M in dioxane) instead of TFA in STEP 2. MS: m/z Calc'd for C₁₈H₂₇N₃O₄ [M+H]⁺350. Found, 350. ¹H NMR (400 MHz, Methanol-d₄) δ 8.56 (s, 2H), 7.27-7.19 (m, 2H), 6.96-6.88 (m, 2H), 4.35 (d, J=4.4 Hz, 1H), 4.89 (s, 1H), 4.14-4.04 (m, 1H), 3.80 (s, 3H), 3.57 (dd, J=12.6, 4.5 Hz, 1H), 3.48-3.35 (m, 2H), 3.33-3.26 (m, 3H), 3.26-3.17 (m, 1H), 3.17-2.93 (m, 3H), 2.70-2.55 (m, 1H), 2.25-2.12 (m, 1H), 1.86-1.72 (m, 1H). Prep-HPLC purification conditions: Column: Welch Ultimate XB-C18 50*250, 10 μm; Mobile Phase A: Water (0.1% FA), Mobile Phase B: ACN; Flow rate: 60 mL/min; Gradient: 0% B to 10% B in 20 min; Wave Length: 254 nm/220 nm; RT1(min): 14.12.

### Synthetic Procedure of V187688

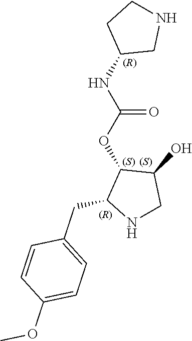

The title compound was prepared in 45.7% overall yield as yellow solid according to Substitution reaction for synthesis of Carbamate; General Procedure II using tert-butyl (R)-3-aminopyrrolidine-1-carboxylate in STEP 1; Boc Deprotection; General Procedure III using HCl (4 M in dioxane) instead of TFA in STEP 2. MS: m/z Calc'd for C₁₇H₂₅N₃O₄ [M+H]⁺336. Found, 336. ¹H NMR (400 MHz, Methanol-d₄) δ 8.54 (s, 1H), 7.26-7.20 (m, 2H), 6.97-6.88 (m, 2H), 4.94 (d, J=3.4 Hz, 2H), 4.39-4.31 (m, 2H), 4.17-4.07 (m, 1H), 3.80 (s, 3H), 3.60-3.44 (m, 3H), 3.44-3.34 (m, 1H), 3.18 (d, J=12.6 Hz, 1H), 3.09 (dd, J=14.1, 7.2 Hz, 1H), 2.98 (dd, J=14.1, 8.4 Hz, 1H), 2.41-2.28 (m, 1H), 2.09-2.07 (m, 1H). Prep-HPLC purification conditions: Column: Welch Ultimate XB-C18 50*250, 10 μm; Mobile Phase A: Water (0.1% FA), Mobile Phase B: ACN; Flow rate: 60 mL/min; Gradient: 0% B to 10% B in 20 min; Wave Length: 254 nm/220 nm; RT1(min): 14.

### Synthetic Procedure of V187599

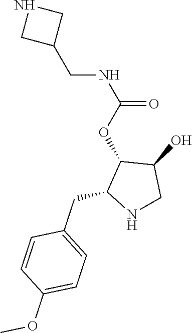 [SMILES: COc1ccc(C[C@H]2NC[C@H](O)[C@H]2OC(=O)NCC2CNC2)cc1]

The title compound was prepared in 38.0% overall yield as a white solid according to Substitution reaction for synthesis of Carbamate; General Procedure II using tert-butyl 3-(aminomethyl)azetidine-1-carboxylate in STEP 1; Boc Deprotection; General Procedure III in STEP 2. MS: m/z Calc'd for C₁₇H₂₅N₃O₄ [M+H]⁺336. Found, 336. ¹H NMR (400 MHz, Methanol-d₄) δ 7.24 (d, J=8.3 Hz, 2H), 6.93 (d, J=8.3 Hz, 2H), 4.96 (d, J=3.4 Hz, 1H), 4.39 (d, J=4.2 Hz, 1H), 4.19-4.11 (m, 3H), 3.97-3.93 (m, 2H), 3.80 (s, 3H), 3.59 (dd, J=12.7, 4.4 Hz, 1H), 3.43 (d, J=6.6 Hz, 2H), 3.22 (d, J=12.6 Hz, 1H), 3.16-3.08 (m, 2H), 2.99 (dd, J=14.1, 8.6 Hz, 1H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 32% B in 10 min; Wave Length: 254 nm/220 nm; RT1(min): 8.68.

### Synthetic Procedure of V187752

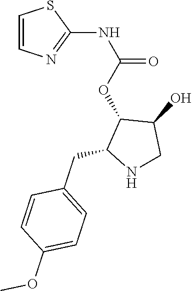 [SMILES: COc1ccc(C[C@H]2NC[C@H](O)[C@H]2OC(=O)Nc2nccs2)cc1]

The title compound was prepared in 46.4% overall yield as a white solid according to Substitution reaction for synthesis of Carbamate; General Procedure II using aminothiazole in STEP 1; Boc Deprotection; General Procedure III in STEP 2. MS: m/z Calc'd for C₁₆H₁₉N₃O₄S [M+H]⁺350. Found, 350. ¹H NMR (400 MHz, DMSO-d₆) δ 10.01 (s, 1H), 9.68 (s, 1H), 7.44 (d, J=3.5 Hz, 1H), 7.30-7.21 (m, 3H), 6.90-6.86 (m, 2H), 4.82 (d, J=3.9 Hz, 1H), 4.36 (d, J=3.9 Hz, 1H), 4.06 (d, J=10.3 Hz, 1H), 3.71-3.69 (m, 3H), 3.58 (s, 1H), 3.48-3.34 (m, 1H), 3.15-2.96 (m, 3H). Prep-HPLC purification conditions: Column: Xselect CSH Prep C18 Column, 30*150 mm, 5 μm; Mobile Phase A: Water (0.05% HCl), Mobile Phase B: ACN; Flow rate: 60 mL/min; Gradient: 2% B to 26% B in 10 min; Wave Length: 254 nm/220 nm; RT1(min): 8.68.

### Synthetic Procedure of V187807

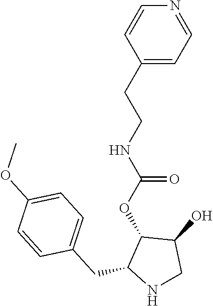 [SMILES: COc1ccc(C[C@H]2NC[C@H](O)[C@H]2OC(=O)NCCc2ccncc2)cc1]

The title compound was prepared in 23.4% overall yield as a light yellow oil according to Substitution reaction for synthesis of Carbamate; General Procedure II using 4-pyridine etheneamine in STEP 1; Boc Deprotection; General Procedure III in STEP 2. MS: m/z Calc'd for C₂₀H₂₅N₃O₄ [M+H]⁺372. found 372. ¹H NMR (400 MHz, Methanol-d₄) δ 8.72 (s, 2H), 7.92 (d, J=5.7 Hz, 2H), 7.20 (d, J=8.2 Hz, 2H), 6.92 (d, J=8.5 Hz, 2H), 4.92 (d, J=3.4 Hz, 2H), 4.34 (d, J=4.2 Hz, 1H), 4.13 (s, 1H), 3.80 (s, 3H), 3.62-3.51 (m, 3H), 3.22-3.11 (m, 3H), 3.05 (dd, J=14.1, 7.0 Hz, 1H), 2.91 (dd, J=14.2, 8.6 Hz, 1H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 8.68.

### Synthetic Procedure of V187812

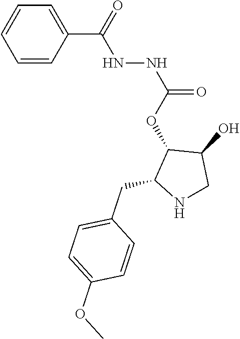 [SMILES: COc1ccc(C[C@H]2NC[C@H](O)[C@H]2OC(=O)NNC(=O)c2ccccc2)cc1]

The title compound was prepared in 42.9% overall yield as a white solid according to Substitution reaction for synthesis of Carbamate; General Procedure II using benzohydrazide in STEP 1; Boc Deprotection; General Procedure III in STEP 2. MS: m/z Calc'd for C₂₀H₂₃N₃O₅ [M+H]⁺386. found 386. ¹H NMR (400 MHz, Methanol-d₄) δ 7.90 (dd, J=7.2, 1.8 Hz, 2H), 7.61 (t, J=7.4 Hz, 1H), 7.51 (t, J=7.6 Hz, 2H), 7.22 (d, J=8.1 Hz, 2H), 6.87 (d, J=8.1 Hz, 2H), 4.71 (d, J=3.5 Hz, 1H), 4.26 (s, 1H), 3.78 (s, 3H), 3.54 (s, 1H), 3.44-3.36 (m, 1H), 2.93-2.67 (m, 3H). Prep-HPLC purification conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 6% B to 36% B in 10 min; Wave Length: 254 nm/220 nm; RT1(min): 8.7.

### Synthetic Procedure of V187821

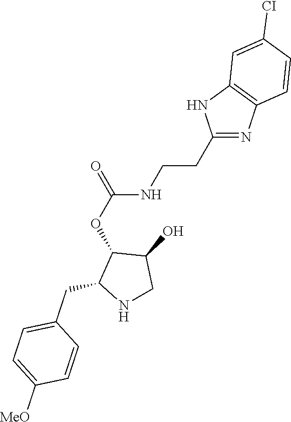

The title compound was prepared in 22.1% overall yield as a white solid according to Substitution reaction for synthesis of Carbamate; General Procedure II using 2-(5-chloro-3H-1,3-benzodiazol-2-yl)ethanamine in STEP 1; Boc Deprotection; General Procedure III in STEP 2. MS: m/z Calc'd for C₂₂H₂₅ClN₄O₄[M+H]⁺445. Found, 445. ¹H NMR (400 MHz, DMSO-d₆) δ 7.78 (d, J=1.9 Hz, 1H), 7.70 (d, J=8.6 Hz, 1H), 7.43 (dd, J=8.6, 2.0 Hz, 1H), 7.15-7.13 (m, 2H), 6.88-6.84 (m, 2H), 4.65 (d, J=3.7 Hz, 1H), 4.15 (d, J=4.1 Hz, 1H), 3.93-3.91 (m, 1H), 3.58-3.51 (m, 2H), 3.34 (dd, J=12.7, 4.3 Hz, 1H), 3.25-3.14 (m, 2H), 3.06 (d, J=12.5 Hz, 1H), 2.84-2.82 (m, 2H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 29% B in 10 min; Wave Length: 254 nm/220 nm; RT1(min): 8.68.

### Synthetic Procedure of V187982

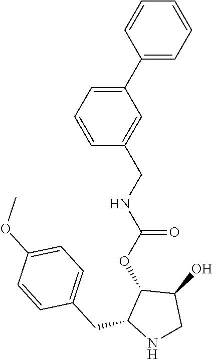 [SMILES: COc1ccc(C[C@H]2NC[C@H](O)[C@H]2OC(=O)NCc2cccc(-c3ccccc3)c2)cc1]

The title compound was prepared in 22.1% overall yield as a white solid according to Substitution reaction for synthesis of Carbamate; General Procedure II using 1-{[1,1′-biphenyl]-3-yl}methanamine in STEP 1; Boc Deprotection; General Procedure III in STEP 2. MS: m/z Calc'd for C₂₆H₂₈N₂O₄ [M+H]⁺432. found 432. ¹H NMR (400 MHz, Methanol-d₄) δ 7.70-7.60 (m, 3H), 7.57 (d, J=7.8 Hz, 1H), 7.50-7.39 (m, 3H), 7.35 (dd, J=7.8, 6.0 Hz, 2H), 7.17 (d, J=8.5 Hz, 2H), 6.82 (d, J=8.5 Hz, 2H), 4.96 (d, J=3.5 Hz, 1H), 4.49-4.47 (m, 1H), 4.41 (d, J=4.2 Hz, 1H), 4.36-4.34 (m, 1H), 4.20-4.11 (m, 1H), 3.74 (s, 3H), 3.58 (dd, J=12.6, 4.4 Hz, 1H), 3.23 (d, J=12.6 Hz, 1H), 3.11 (dd, J=14.0, 7.6 Hz, 1H), 2.96 (dd, J=14.0, 8.2 Hz, 1H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 18% B to 48% B in 10 min; Wave Length: 254 nm/220 nm; RT1(min): 8.35.

### Synthetic Procedure of V187600

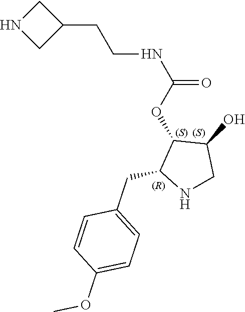

The title compound was prepared in 51.4% overall yield as an off-white semi-solid according to Substitution reaction for synthesis of Carbamate; General Procedure II using tert-butyl 3-(2-aminoethyl)azetidine-1-carboxylate in STEP 1; Boc Deprotection; General Procedure III in STEP 2. MS: m/z Calc'd for C₁₈H₂₇NO₄[M+H]⁺350, Found 350. ¹H NMR (400 MHz, Methanol-d₄) δ 7.27-7.20 (m, 2H), 6.97-6.89 (m, 2H), 4.95 (s, 1H), 4.38 (d, 1H), 4.16-4.14 (m, 3H), 3.85-3.80 (m, 3H), 3.58-3.56 (m, 1H), 3.28-3.06 (m, 4H), 3.09-2.93 (m, 2H), 1.91 (s, 2H). Prep-HPLC purification conditions: Column: Welch Ultimate AQ-C18 50*250, 10 μm; Mobile Phase A: Water (0.05% TFA), Mobile Phase B: ACN; Flow rate: 60 mL/min; Gradient: 2% B to 35% B in 20 min; Wave Length: 254 nm/220 nm; RT1(min): 8.5.

### Synthetic Procedure of V187755

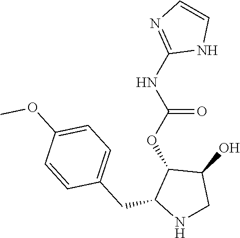 [SMILES: COc1ccc(C[C@H]2NC[C@H](O)[C@H]2OC(=O)Nc2ncc[nH]2)cc1]

The title compound was prepared in 9.6% overall yield as a white solid according to Substitution reaction for synthesis of Carbamate; General Procedure II using 1H-imidazol-2-amine in STEP 1; Boc Deprotection; General Procedure III in STEP 2. MS: m/z Calc'd for C₁₆H₂₀N₄O₄ [M+H]⁺333. found 333. ¹H NMR (400 MHz, Methanol-d₄) δ 7.30-7.05 (m, 2H), 6.98-6.72 (m, 2H), 6.52 (s, 2H), 4.62 (d, J=3.7 Hz, 1H), 4.35-4.09 (m, 1H), 3.77 (d, J=5.9 Hz, 3H), 3.69-3.36 (m, 2H), 3.24-2.54 (m, 3H), 1.77-1.20 (m, 1H). Prep-HPLC purification conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 3% B to 30% B in 10 min; Wave Length: 254 nm/220 nm; RT1(min): 9.2.

### Synthetic Procedure of V187815

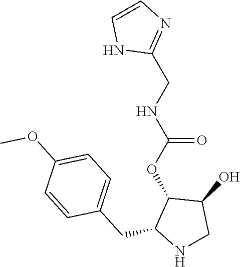 [SMILES: COc1ccc(C[C@H]2NC[C@H](O)[C@H]2OC(=O)NCc2ncc[nH]2)cc1]

The title compound was prepared in 34.5% overall yield as a yellow solid according to Substitution reaction for synthesis of Carbamate; General Procedure II using 1-(1H-imidazol-2-yl)methanamine in STEP 1; Boc Deprotection; General Procedure III in STEP 2. MS: m/z Calc'd for C₁₇H₂₂N₄O₄ [M+H]⁺347. found 347. ¹H NMR (400 MHz, Methanol-d₄) δ 7.51 (s, 2H), 7.26-7.19 (m, 2H), 6.90 (d, J=8.3 Hz, 2H), 4.99 (d, J=3.3 Hz, 1H), 4.66 (d, J=2.0 Hz, 2H), 4.41 (d, J=4.2 Hz, 1H), 4.17-4.05 (m, 1H), 3.79 (s, 3H), 3.61-3.48 (m, 1H), 3.22 (d, J=12.6 Hz, 1H), 3.11 (dd, J=14.2, 6.9 Hz, 1H), 3.00-2.89 (m, 1H). Prep-HPLC purification conditions: Column: XBridge Prep OBD C18 Column, 19*250 mm, 5 μm; Mobile Phase A: Water (0.05% TFA), Mobile Phase B: ACN; Flow rate: 25 mL/min; Gradient: 3% B to 26% B in 8 min; Wave Length: 254 nm/220 nm; RT1(min): 6.88.

### Synthetic Procedure of V187816

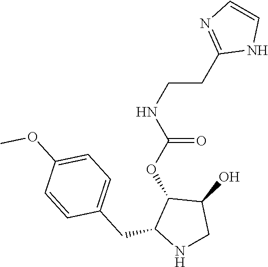 [SMILES: COc1ccc(C[C@H]2NC[C@H](O)[C@H]2OC(=O)NCCc2ncc[nH]2)cc1]

The title compound was prepared in 33.3% overall yield as a white solid according to Substitution reaction for synthesis of Carbamate; General Procedure II using 2-(1H-imidazol-2-yl)ethanamine in STEP 1; Boc Deprotection; General Procedure III in STEP 2. MS: m/z Calc'd for C₁₈H₂₄N₄O₄ [M+H]⁺361. found 361. ¹H NMR (400 MHz, Methanol-d₄) (7.46 (d, J=1.9 Hz, 2H), 7.29-7.17 (m, 2H), 6.92 (d, J=8.4 Hz, 2H), 4.90 (s, 1H), 4.32 (d, J=4.1 Hz, 1H), 4.17-4.09 (m, 1H), 3.79 (s, 3H), 3.63-3.51 (m, 3H), 3.26-3.16 (m, 3H), 3.05 (dd, J=14.2, 6.8 Hz, 1H), 2.92-2.79 (m, 1H). Prep-HPLC purification conditions: Column: XBridge Prep OBD C18 Column, 19*250 mm, 5 μm; Mobile Phase A: Water (0.05% TFA), Mobile Phase B: ACN; Flow rate: 25 mL/min; Gradient: 3% B to 26% B in 8 min; Wave Length: 254 nm/220 nm; RT1(min): 7.23.

### Synthetic Procedure of V187818

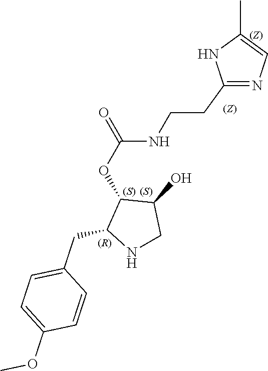

The title compound was prepared in 13.5% overall yield as a white solid according to Substitution reaction for synthesis of Carbamate; General Procedure II using 2-(4-methyl-3H-imidazol-2-yl)ethanamine in STEP 1; Boc Deprotection; General Procedure III in STEP 2. MS: m/z Calc'd for C₁₉H₂₆N₄O₄[M+H]⁺375, Found 375. ¹H NMR (400 MHz, Methanol-d₄) δ 7.28-7.18 (m, 2H), 7.14 (d, 1H), 6.97-6.88 (m, 2H), 4.97-4.91 (m, 1H), 4.36-4.31 (m, 1H), 4.14 (s, 1H), 3.80 (s, 3H), 3.57-3.55 (m, 3H), 3.24-3.12 (m, 3H), 3.06-3.04 (m, 1H), 2.91-2.89 (m, 1H), 2.31 (s, 3H). Prep-HPLC purification conditions: Column: XBridge Prep OBD C18 Column, 19*250 mm, 5 μm; Mobile Phase A: Water (0.05% TFA), Mobile Phase B: ACN; Flow rate: 25 mL/min; Gradient: 3% B to 26% B in 8 min; Wave Length: 254 nm/220 nm; RT1(min): 7.23.

### Synthetic Procedure of V187819

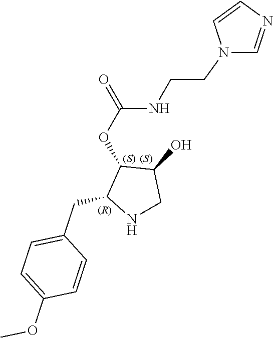

The title compound was prepared in 55.3% overall yield as a yellow oil according to Substitution reaction for synthesis of Carbamate; General Procedure II using O-aminoethylimidazole in STEP 1; Boc Deprotection; General Procedure III in STEP 2. MS: m/z Calc'd for C₁₈H₂₄N₄O₄[M+H]⁺361, Found 361. ¹H NMR (400 MHz, Methanol-d₄) δ 8.93 (s, 1H), 7.65 (s, 1H), 7.55 (s, 1H), 7.25-7.18 (m, 2H), 6.98-6.89 (m, 2H), 4.40 (d, 2H), 4.35 (d, 2H), 4.15-4.13 (m, 1H), 3.80 (s, 3H), 3.65 (s, 2H), 3.56-3.54 (m, 1H), 3.31 (s, 1H), 3.21 (d, 1H), 3.07-3.05 (m, 1H), 2.92-2.90 (m, 1H). Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 5% B to 95% B in 20 min; Wave Length: 254 nm/220 nm; RT1(min): 11.26.

### Synthetic Procedure of V187820

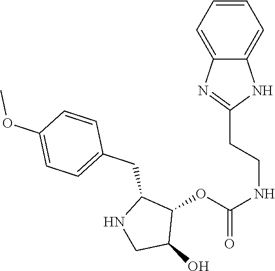 [SMILES: COc1ccc(C[C@H]2NC[C@H](O)[C@H]2OC(=O)NCCc2nc3ccccc3[nH]2)cc1]

The title compound was prepared in 32.4% overall yield as a white solid according to Substitution reaction for synthesis of Carbamate; General Procedure II using 2-(1H-1,3-benzodiazol-2-yl)ethanamine in STEP 1; Boc Deprotection; General Procedure III in STEP 2. MS: m/z Calc'd for C₂₂H₂₆N₄O₄ [M+H]⁺411. found 411. ¹H NMR (400 MHz, Methanol-d₄) δ 7.77-7.68 (m, 2H), 7.53-7.41 (m, 2H), 7.15 (d, J=8.2 Hz, 2H), 6.92-6.84 (m, 2H), 4.29-4.22 (m, 1H), 4.07-3.98 (m, 1H), 3.78 (s, 3H), 3.74-3.61 (m, 2H), 3.53-3.43 (m, 1H), 3.41-3.31 (m, 2H), 3.20-3.12 (m, 1H), 2.99-2.89 (m, 1H), 2.84-2.71 (m, 1H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 24% B in 10 min; Wave Length: 254 nm/220 nm; RT1(min): 8.68.

### Synthetic Procedure of V187862

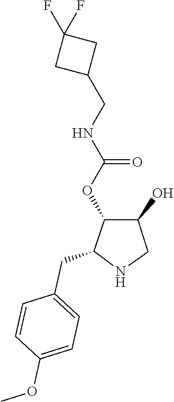 [SMILES: COc1ccc(C[C@H]2NC[C@H](O)[C@H]2OC(=O)NCC2CC(F)(F)C2)cc1]

The title compound was prepared in 32.4% overall yield as a white solid according to Substitution reaction for synthesis of Carbamate; General Procedure II using 1-(3,3-difluorocyclobutyl)methanamine hydrochloride in STEP 1; Boc Deprotection; General Procedure III in STEP 2. MS: m/z Calc'd for C₁₈H₂₄F₂NO₄[M+H]⁺371, Found 371. ¹H NMR (400 MHz, Methanol-d₄) δ 7.23 (dd, 2H), 6.97-6.89 (m, 2H), 4.93 (s, 1H), 4.42-4.33 (m, 1H), 4.21-4.13 (m, 1H), 3.78 (s, 3H), 3.56 (s, 1H), 3.32-3.18 (m, 3H), 3.09 (s, 1H), 2.97-2.95 (m, 1H), 2.71-2.58 (m, 2H), 2.41-2.30 (m, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 8% B to 38% B in 10 min; Wave Length: 254 nm/220 nm; RT1(min): 8.68.

### Synthetic Procedure of V187877

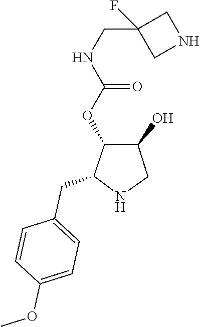 [SMILES: COc1ccc(C[C@H]2NC[C@H](O)[C@H]2OC(=O)NCC2(F)CNC2)cc1]

The title compound was prepared in 31.9% overall yield as a white solid according to Substitution reaction for synthesis of Carbamate; General Procedure II using tert-butyl 3-(aminomethyl)-3-fluoroazetidine-1-carboxylate in STEP 1; Boc Deprotection; General Procedure III in STEP 2. MS: m/z Calc'd for C₁₇H₂₄FNO₄[M+H]⁺354, Found 354. ¹H NMR (400 MHz, Methanol-d₄) δ 7.14 (d, 2H), 6.84-6.82 (m, 2H), 4.63-4.61 (m, 1H), 4.15-4.11 (m, 1H), 3.78 (s, 4H), 3.81-3.48 (m, 6H), 3.38-3.31 (m, 1H), 2.88-2.66 (m, 3H). Prep-HPLC purification conditions: Column: XBridge Prep OBD C18 Column, 50*250 mm, 10 μm; Mobile Phase A: Water (10 mmol/LNH₄HCO₃), Mobile Phase B: ACN; Flow rate: 60 mL/min; Gradient: 2% B to 35% B in 20 min; Wave Length: 254 nm/220 nm; RT1(min): 18.08.

### Synthetic Procedure of V187916

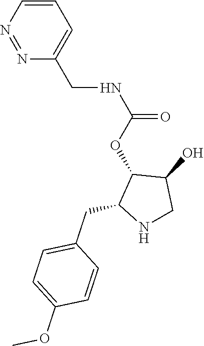 [SMILES: COc1ccc(C[C@H]2NC[C@H](O)[C@H]2OC(=O)NCc2cccnn2)cc1]

The title compound was prepared in 16.4% overall yield as a yellow oil according to Substitution reaction for synthesis of Carbamate; General Procedure II using 1-(pyridazin-3-yl)methanamine in STEP 1; Boc Deprotection; General Procedure III in STEP 2. MS: m/z Calc'd for C₁₈H₂₂N₄O₄[M+H]⁺359, Found 359. ¹H NMR (400 MHz, Methanol-d₄) δ 9.19-9.13 (m, 1H), 7.77-7.75 (m, 2H), 7.24-7.22 (m, 2H), 6.91-6.89 (m, 2H), 4.97-4.95 (m, 1H), 4.67 (s, 2H), 4.43-4.41 (m, 1H), 4.18-4.16 (m, 1H), 3.80 (s, 3H), 3.62-6.60 (m, 1H), 3.24-3.22 (m, 1H), 3.14-3.12 (m, 1H), 3.00-2.98 (m, 1H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 27% B in 10 min; Wave Length: 254 nm/220 nm; RT1(min): 8.68.

### Synthetic Procedure of V187917

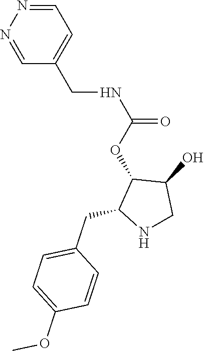 [SMILES: COc1ccc(C[C@H]2NC[C@H](O)[C@H]2OC(=O)NCc2ccnnc2)cc1]

The title compound was prepared in 37.8% overall yield as a brown oil according to Substitution reaction for synthesis of Carbamate; General Procedure II using 1-(pyridazin-4-yl)methanamine in STEP 1; Boc Deprotection; General Procedure III in STEP 2. MS: m/z Calc'd for C₁₈H₂₂N₄O₄[M+H]⁺359, Found 359. ¹H NMR (400 MHz, Methanol-d₄) δ 9.26-9.18 (m, 2H), 7.81-7.72 (m, 1H), 7.22-7.20 (m, 2H), 6.90-6.88 (m, 2H), 4.96-4.98 (m, 1H), 4.47-4.42 (m, 3H), 4.20-4.18 (m, 1H), 3.80 (s, 3H), 3.61-3.59 (m, 1H), 3.25-3.23 (m, 1H), 3.13-3.11 (m, 1H), 3.01-2.99 (m, 1H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 18% B in 10 min; Wave Length: 254 nm/220 nm; RT1(min): 8.68.

### Synthetic Procedure of V187928

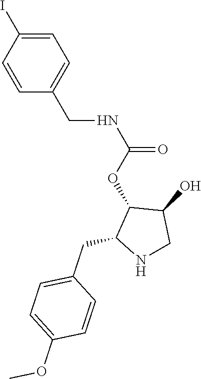 [SMILES: COc1ccc(C[C@H]2NC[C@H](O)[C@H]2OC(=O)NCc2ccc(I)cc2)cc1]

The title compound was prepared in 25.9% overall yield as a white solid according to Substitution reaction for synthesis of Carbamate; General Procedure II using 1-(4-iodophenyl)methanamine in STEP 1; Boc Deprotection; General Procedure III in STEP 2. MS: m/z Calc'd for C₂₀H₂₃IN-204[M+H]⁺483, Found 483. ¹H NMR (400 MHz, Methanol-d₄) δ 7.73-7.71 (m, 2H), 7.16-7.14 (m, 4H), 6.87-6.85 (m, 2H), 4.92-4.90 (m, 1H), 4.60 (s, 1H), 4.39-4.37 (m, 2H), 4.33-4.31 (m, 1H), 3.80 (s, 3H), 3.58-3.56 (m, 1H), 3.23-3.21 (m, 1H), 2.97-2.95 (m, 2H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 15% B to 45% B in 10 min; Wave Length: 254 nm/220 nm; RT1(min): 8.35.

### Synthetic Procedure of V187929

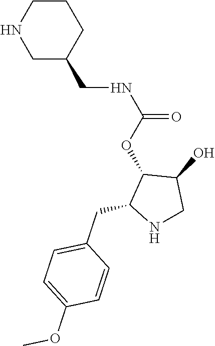 [SMILES: COc1ccc(C[C@H]2NC[C@H](O)[C@H]2OC(=O)NC[C@@H]2CCCNC2)cc1]

The title compound was prepared in 25.9% overall yield as a white semi-solid according to Substitution reaction for synthesis of Carbamate; General Procedure II using tert-butyl (3S)-3-(aminomethyl)piperidine-1-carboxylate in STEP 1; Boc Deprotection; General Procedure III in STEP 2. MS: m/z Calc'd for C₁₉H₂₉N₄O₄[M+H]⁺364, Found 364. ¹H NMR (400 MHz, Methanol-d₄) δ 7.28-7.20 (m, 2H), 6.97-6.89 (m, 2H), 4.94-4.92 (m, 1H), 4.38-4.36 (m, 1H), 4.17-4.15 (m, 1H), 3.80 (s, 3H), 3.58-3.56 (m, 1H), 3.37-3.35 (m, 2H), 3.26-3.06 (m, 4H), 3.04-2.88 (m, 2H), 2.79-2.65 (m, 1H), 2.02-1.91 (m, 3H), 1.83-1.68 (m, 1H), 1.40-1.26 (m, 1H). Prep-HPLC purification conditions: Column: Welch Ultimate AQ-C18 50*250, 10 μm; Mobile Phase A: Water (0.05% TFA), Mobile Phase B: ACN; Flow rate: 60 mL/min; Gradient: 2% B to 40% B in 20 min; Wave Length: 254 nm/220 nm; RT1(min): 17.08.

### Synthetic Procedure of V187930

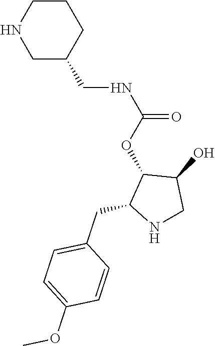 [SMILES: COc1ccc(C[C@H]2NC[C@H](O)[C@H]2OC(=O)NC[C@H]2CCCNC2)cc1]

The title compound was prepared in 25.9% overall yield as a white solid according to Substitution reaction for synthesis of Carbamate; General Procedure II using tert-butyl (3R)-3-(aminomethyl)piperidine-1-carboxylate in STEP 1; Boc Deprotection; General Procedure III in STEP 2. MS: m/z Calc'd for C₁₉H₂₉NO₄[M+H]⁺364, Found 364. ¹H NMR (400 MHz, Methanol-d₄) δ 7.24-7.22 (m, 2H), 6.93-6.91 (m, 2H), 4.96-4.94 (m, 1H), 4.39-4.35 (m, 1H), 4.17-4.15 (m, 1H), 3.80 (s, 3H), 3.59-3.57 (m, 1H), 3.43-3.35 (m, 2H), 3.26-3.07 (m, 4H), 3.04-2.87 (m, 2H), 2.78-2.66 (m, 1H), 2.08-1.90 (m, 3H), 1.83-1.68 (m, 1H), 1.33-1.31 (m, 1H). Prep-HPLC purification conditions: Column: Welch Ultimate AQ-C18 50*250, 10 μm; Mobile Phase A: Water (0.05% TFA), Mobile Phase B: ACN; Flow rate: 60 m/min; Gradient: 2% B to 25% B in 20 min; Wave Length: 254 nm/220 nm; RT1(min): 20.7.

### Synthetic Procedure of V187719

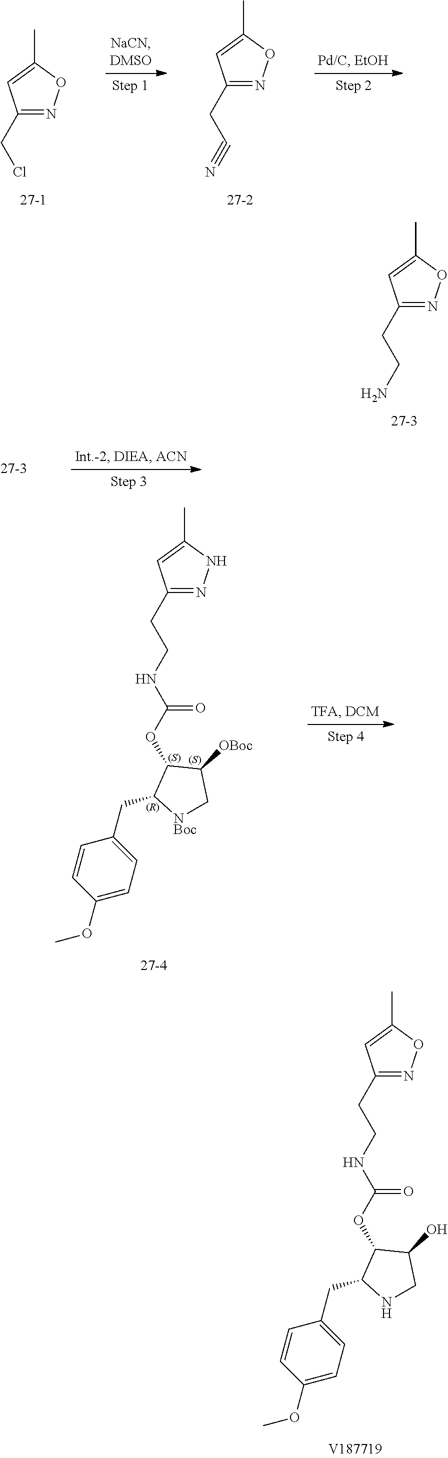

Step 1: To a stirred solution of 3-(chloromethyl)-5-methyl-1,2-oxazole (27-3, 1 g, 7.60 mmol, 1.0 eq.) in DMSO (10 mL) was added NaCN (745 mg, 15.202 mmol, 2.0 eq.) in portions at 0° C. The resulting mixture was stirred at 25° C. for overnight. LCMS showed the reaction was completed. The reaction was quenched with Water at 0° C. The aqueous layer was extracted with EtOAc (3×3 mL). Combined the organic phase, dried over anhydrous sodium sulfate. After filtration, the filtrate was concentrated under reduced pressure to afford 2-(5-methyl-1,2-oxazol-3-yl)acetonitrile (700 mg, 75.4%) as a light yellow solid which was used directly in the next step without further purification. MS: m/z: Calc'd for C₆H₆N₂O [M+H]⁺123. Found, 123.

Step 2: To a stirred solution of 2-(5-methyl-1,2-oxazol-3-yl)acetonitrile (27-2, 100 mg, 0.82 mmol, 1.0 eq.) in EtOH (5 mL) was added Pd/C (100 mg, 0.94 mmol, 1.2 eq.) in portions at room temperature under N₂. The reaction was placed under vacuum, sonicated and backfilled with H₂. The resulting mixture was stirred 1 h at 25° C. LCMS showed the reaction was completed. The resulting mixture was filtered, the filter cake was washed with EtOH (2×1 mL). The filtrate was concentrated under reduced pressure. The crude product was used in the next step directly without further purification. MS: m/z: Calc'd for C₆H₁₀N₂O [M+H]⁺127. Found, 127.

Step 3 & 4: The title compound was prepared in 24.0% overall yield as a light yellow semi-solid according to Substitution reaction for synthesis of Carbamate; General Procedure II using 2-(5-methyl-1,2-oxazol-3-yl)ethanamine (27-3) in STEP 3; Boc Deprotection; General Procedure III in STEP 4. MS: m/z Calc'd for C₁₉H₂₅N₃O₅ [M+H]⁺376. Found, 376. ¹H NMR (400 MHz, Methanol-d₄) δ 7.22 (d, J=8.3 Hz, 2H), 6.93 (d, J=8.3 Hz, 2H), 6.11 (s, 1H), 4.91 (d, J=3.5 Hz, 1H), 4.36 (d, J=4.3 Hz, 1H), 4.18-4.10 (m, J=7.8, 3.4 Hz, 1H), 3.80 (s, 3H), 3.61-3.40 (m, 3H), 3.21 (d, J=12.6 Hz, 1H), 3.07 (dd, J=14.1, 7.3 Hz, 1H), 2.98-2.80 (m, 3H), 2.38 (s, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 34% B to 55% B in 10 min; Wave Length: 254 nm/220 nm; RT1(min): 8.68.

### Synthetic Procedure of V187824

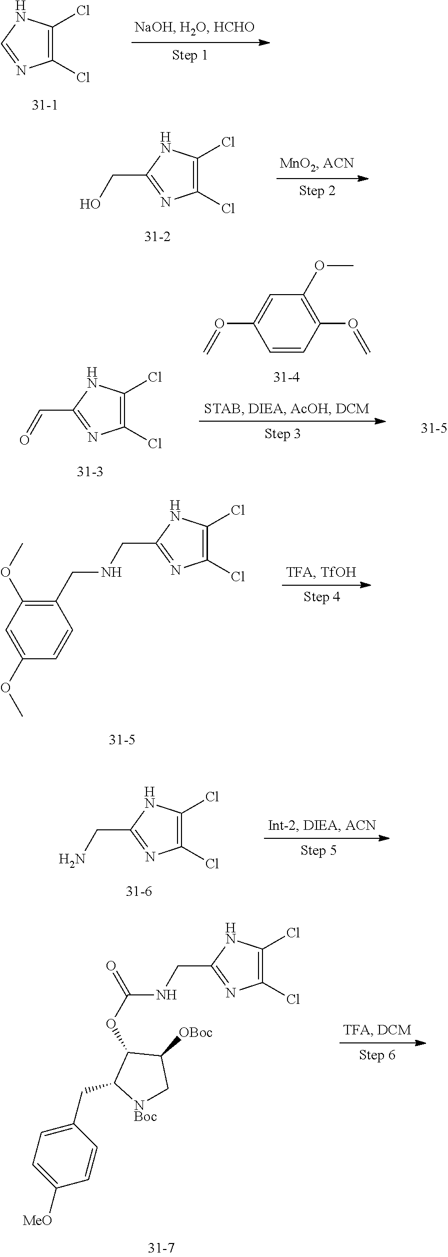

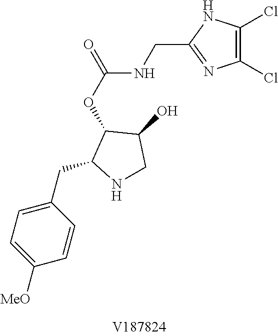 [SMILES: COc1ccc(C[C@H]2NC[C@H](O)[C@H]2OC(=O)NCc2nc(Cl)c(Cl)[nH]2)cc1]

Step 1: To a stirred solution of 4,5-dichloro-1H-imidazole (31-1, 500 mg, 3.65 mmol, 1.0 eq.) in H₂O (3.6 mL) was added NaOH (146.02 mg, 3.65 mmol, 1.0 eq.) and HCHO (109.6 mg, 3.65 mmol, 1.0 eq.) in portions at room temperature. The resulting mixture was stirred at room temperature for 2 h. Desired product could be detected by LCMS. The PH was adjusted to 4 with HCl (2 mol/L) at 0° C. The solids were collected by filtration to give (4,5-dichloro-1H-imidazol-2-yl)methanol (470 mg, 77.1%) as a white solid, which was used in the next step directly without further purification. MS: m/z: Calc'd for C₄H₄Cl₂N₂O [M+H]⁺167. Found, 167.

Step 2: To a stirred solution of (4,5-dichloro-1H-imidazol-2-yl)methanol (31-2, 250 mg, 1.5 mmol, 1.0 eq.) in ACN (1 mL) was added MnO₂ (650.8 mg, 7.49 mmol, 5.0 eq.) in portions at room temperature. The resulting mixture was stirred over night at 60° C. After completion of reaction monitored by LCMS. The resulting mixture was filtered, the filter cake was washed with MeCN (1×3 mL). The filtrate was concentrated under reduced pressure to afford 4,5-dichloro-1H-imidazole-2-carbaldehyde (240 mg, 97.2%) as a yellow oil, which was used in the next step directly without further purification. MS: m/z: Calc'd for C₄H₂Cl₂N₂O [M+H]⁺165. Found, 165.

Step 3: To a stirred solution of 4,5-dichloro-1H-imidazole-2-carbaldehyde (31-3, 200 mg, 1.21 mmol, 1.0 eq.) and 2,4-dimethoxybenzaldehyde (31-4, 201.5 mg, 1.21 mmol, 1.0 eq.) in DCM (5 mL) was added DIEA (470 mg, 3.64 mmol, 3.0 eq.) and AcOH (291.2 mg, 4.85 mmol, 4.0 eq.) at room temperature. After 0.5 h STAB (770.8 mg, 3.64 mmol, 3.0 eq.) was added at 0° C. The resulting mixture was stirred over night at room temperature. After completion of reaction monitored by LCMS. The reaction was quenched with Water at 0° C. The aqueous layer was extracted with DCM (3×3 mL). The resulting mixture was concentrated under reduced pressure. The residue was purified by reversed-phase flash chromatography to afford [(4,5-dichloro-1H-imidazol-2-yl)methyl][(2,4-dimethoxyphenyl)methyl]amine (300 mg, 78.2%) as a white solid. MS: m/z: Calc'd for C₁₃H₁₅Cl₂N₃O₂ [M+H]⁺316. Found, 316.

Step 4: To a stirred solution of [(4,5-dichloro-1H-imidazol-2-yl)methyl][(2,4-dimethoxyphenyl)methyl]amine (31-5, 270 mg, 0.854 mmol, 1 eq.) in TFA (3 mL) was added trifluoromethanesulfonic acid (0.3 mL) at 0° C. The resulting mixture was stirred at room temperature for 2 h. The resulting mixture was concentrated under reduced pressure. The crude product was used in the next step directly without further purification. MS: m/z: Calc'd for C₄H₅Cl₂N₃[M+H]⁺ 166. Found, 166.

Step 5 & 6: The title compound was prepared in 31.2% overall yield as a white solid according to Substitution reaction for synthesis of Carbamate; General Procedure II using 1-(4,5-dichloro-1H-imidazol-2-yl)methanamine (31-6) in STEP 5; Boc Deprotection; General Procedure III in STEP 6. MS: m/z Calc'd for C₁₇H₂₀Cl₂N₄O₄ [M+H]⁺415. Found, 415. ¹H NMR (400 MHz, Methanol-d₄) δ 7.25-7.09 (m, 2H), 6.90 (dd, J=8.4, 1.8 Hz, 2H), 4.95 (d, J=3.3 Hz, 1H), 4.42 (d, J=4.2 Hz, 1H), 4.31 (s, 2H), 4.18-4.14 (m, 1H), 3.79 (s, 3H), 3.62-3.58 (m, 1H), 3.22 (d, J=12.6 Hz, 1H), 3.11 (dd, J=14.1, 7.3 Hz, 1H), 3.04-2.92 (m, 1H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 5% B to 35% B in 10 min; Wave Length: 254 nm/220 nm; RT1(min): 8.57.

### Synthetic Procedure of V187823

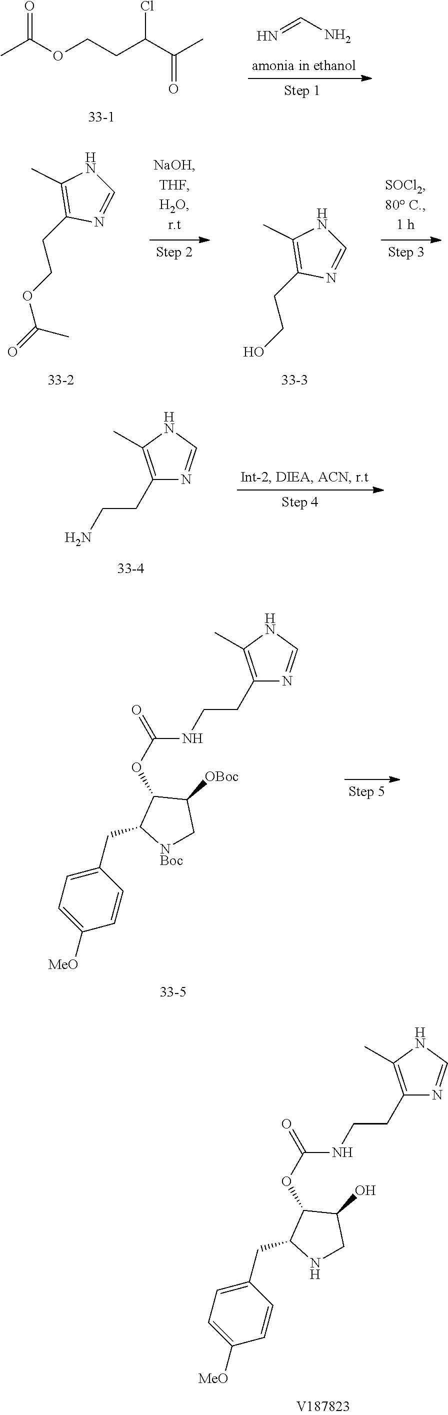

Step 1: To a stirred mixture of 3-chloro-4-oxopentyl acetate (33-1, 1 g, 5.59 mmol, 1 equiv) in EtOH (10 mL) was added formamidine (0.25 g, 5.59 mmol, 1 equiv) at room temperature. The resulting mixture was stirred at 80° C. for overnight. The resulting mixture was concentrated under reduced pressure. The residue was purified by reversed-phase flash chromatography to afford 2-(5-methyl-1H-imidazol-4-yl)ethyl acetate (500 mg, 53.1%) as a yellow oil. MS: m/z: Calc'd for C₈H₁₂N₂O₂ [M+H]⁺169. found 169.

Step 2: To a stirred mixture of 2-(5-methyl-1H-imidazol-4-yl)ethyl acetate (33-2, 300 mg, 1.784 mmol, 1 equiv) in THF (7.5 mL) and water (2.5 mL) was added NaOH (214.1 mg, 5.35 mmol, 3 equiv) at room temperature. The resulting mixture was stirred at room temperature for overnight. Upon completion, the mixture was concentrated, the residue was purified by reversed-phase flash chromatography to afford 2-(5-methyl-1H-imidazol-4-yl)ethanol (100 mg, 44.4%) as a yellow oil. MS: m/z: Calc'd for C₆H₁₀N₂O [M+H]⁺127. found 127.

Step 3: To a stirred mixture of 2-(5-methyl-1H-imidazol-4-yl)ethanol (33-3, 300 mg, 2.38 mmol, 1 equiv) was added SOCl₂ (10 mL) at room temperature under. The resulting mixture was stirred at 80° C. for overnight. The resulting mixture was concentrated under reduced pressure. The residue was added Ammonia at room temperature under nitrogen atmosphere. The resulting mixture was stirred at 80° C. for overnight. Upon completion, the mixture was concentrated in vacuum. The crude was used in the next step directly without further purification. MS: m/z: Calc'd for C₆H₁₁N₃ [M+H]⁺126. found 126.

Step 4 & 5: The title compound was prepared in 21.5% overall yield as a yellow solid according to Substitution reaction for synthesis of Carbamate; General Procedure II using 4-methylhistamine (33-4) in STEP 4; Boc Deprotection; General Procedure III in STEP 5. MS: m/z Calc'd for C₁₉H₂₆N₄O₄ [M+H]⁺375. found 375. ¹H NMR (400 MHz, Methanol-d₄) δ 8.71 (s, 1H), 7.22 (d, J=8.2 Hz, 2H), 6.92 (d, J=8.3 Hz, 2H), 4.94 (d, J=3.5 Hz, 1H), 4.33 (d, J=4.2 Hz, 1H), 4.14-4.02 (m, 1H), 3.79 (s, 3H), 3.56 (dd, J=12.7, 4.3 Hz, 1H), 3.43-3.32 (m, 2H), 3.20 (d, J=12.7 Hz, 1H), 3.07 (dd, J=14.2, 6.7 Hz, 1H), 2.92-2.83 (m, 3H), 2.32 (s, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 9.13.

### Synthetic Procedure of V187826

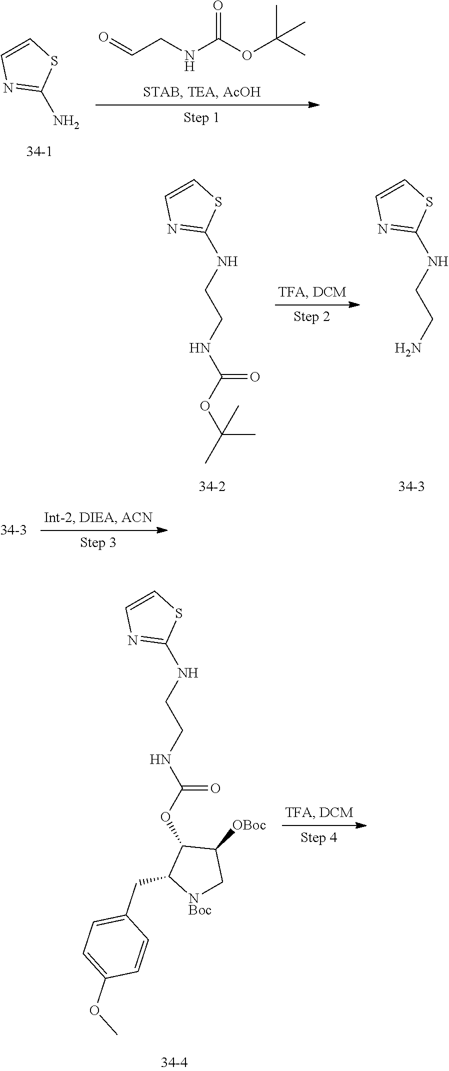

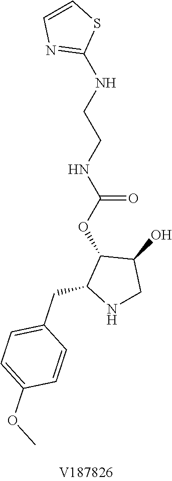 [SMILES: COc1ccc(C[C@H]2NC[C@H](O)[C@H]2OC(=O)NCCNc2nccs2)cc1]

Step 1: To a stirred solution of aminothiazole (500 mg, 4.99 mmol, 1 eq.) and tert-butyl N-(2-oxoethyl)carbamate (1.6 g, 9.99 mmol, 2 eq.) in MeOH (100 mL) was treated with AcOH (10 mL) for 1 h at 0° C. under nitrogen atmosphere followed by the addition of (2-methylpyridin-1-ium-1-yl)boranuide (2.1 g, 19.97 mmol, 4 eq.) at 0° C. The resulting mixture was stirred at room temperature for 1 h. After completion of reaction monitored by LCMS. The reaction was quenched with Water at 0° C. The resulting mixture was concentrated under reduced pressure. The residue was purified by flash. This resulted in tert-butyl N-[2-(1,3-thiazol-2-ylamino)ethyl]carbamate (190 mg, 15.6%) as a light yellow oil. MS: m/z: Calc'd for C₁₀H₁₇N₃O₂S [M+H]⁺244. found [M+H]⁺ 244.

Step 2: To a stirred solution of tert-butyl N-[2-(1,3-thiazol-2-ylamino)ethyl]carbamate (34-2, 180 mg, 0.74 mmol) in DCM (5 mL) was added TFA (1 mL). The resulting mixture was stirred at room temperature for 1 h. After completion of reaction monitored by LCMS, concentrated, the crude product was used in the next step directly without further purification. MS: m/z: Calc'd for C₅H₉N₃S [M+H]⁺144. found 144.

Step 3 & 4: The title compound was prepared in 32.4% overall yield as an off-white solid according to Substitution reaction for synthesis of Carbamate; General Procedure II using N1-(1,3-thiazol-2-yl)ethane-1,2-diamine (34-3) in STEP 3; Boc Deprotection; General Procedure III in STEP 4. MS: m/z Calc'd for C₁₈H₂₄N₄O₄S [M+H]⁺393. found 393. ¹H NMR (400 MHz, Methanol-d₄) δ 7.28-7.18 (m, 3H), 6.97-6.88 (m, 2H), 6.86 (d, J=4.2 Hz, 1H), 5.00-4.95 (m, 1H), 4.40-4.34 (m, 1H), 4.18-4.14 (m, 1H), 3.79 (s, 3H), 3.59-3.44 (m, 5H), 3.21 (d, J=12.6 Hz, 1H), 3.09 (dd, J=14.2, 6.8 Hz, 1H), 2.94 (dd, J=14.2, 8.7 Hz, 1H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 8.68.

### Synthetic Procedure of V187847

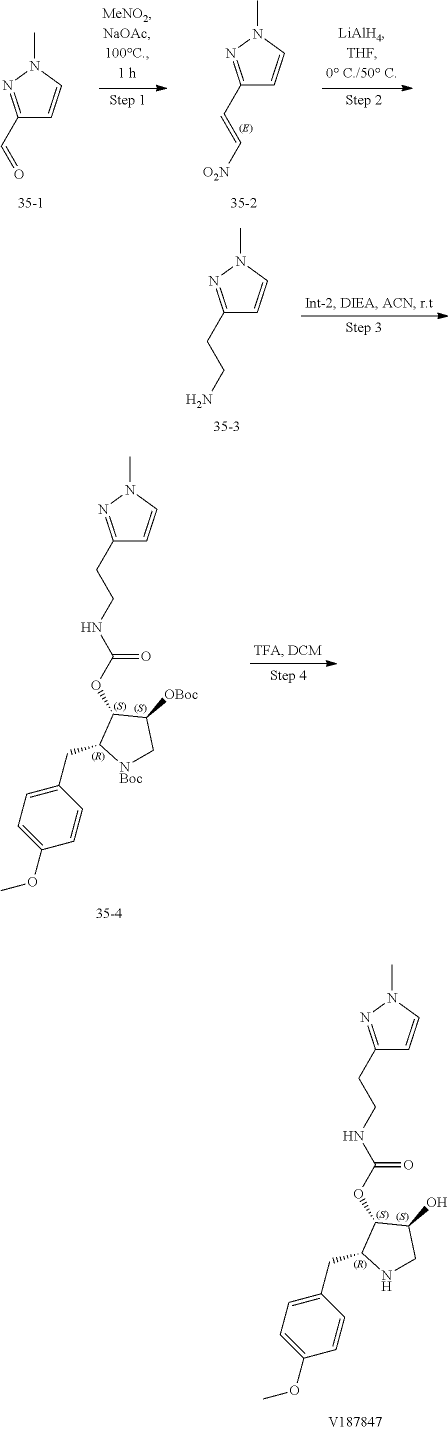

Step 1: To a stirred mixture of 1-methylpyrazole-3-carbaldehyde (1 g, 9.10 mmol, 1 eq.) in nitromethane (10 mL) was added ammonium acetate (1.05 g, 13.62 mmol, 1.5 eq.) at room temperature. The resulting mixture was stirred for overnight at 110° C. The resulting mixture was concentrated under reduced pressure. The residue was purified by silica gel column chromatography, eluted with PE/EA (3:1) to afford 1-methyl-3-[(E)-2-nitroethenyl]pyrazole (150 mg, 10.8%) as a yellow oil. MS: m/z: Calc'd for C₆H₇N₃O₂[M+H]⁺154. found 154.

Step 2: To a stirred mixture of 1-methyl-3-[(E)-2-nitroethenyl]pyrazole (100 mg, 0.65 mmol, 1 eq.) in THF (5 mL) was added LiAlH₄ (1 mL, 0.98 mmol, 1.5 eq.) at 0° C. The resulting mixture was stirred at 50° C. for overnight. The reaction was quenched with water/EA at 0° C. The precipitated solids were collected by filtration and washed with EtOAc (2×20 mL). The resulting mixture was concentrated under vacuum to afford crude product as a yellow oil. MS: m/z: Calc'd for C₆H₁₁N3 [M+H]⁺126. found 126.

Step 3 & 4: The title compound was prepared in 6.6% overall yield as a yellow solid according to Substitution reaction for synthesis of Carbamate; General Procedure II using 2-(1-methylpyrazol-3-yl)ethanamine (35-3) in STEP 3; Boc Deprotection; General Procedure III in STEP 4. MS: m/z Calc'd for C₁₉H₂₆N₄O₄ [M+H]⁺375. found 375. ¹H NMR (400 MHz, Methanol-d₄) δ 7.49 (d, J=2.2 Hz, 1H), 7.25-7.17 (m, 2H), 6.96-6.88 (m, 2H), 6.16 (d, J=2.2 Hz, 1H), 4.96-4.91 (m, 1H), 4.37 (d, J=4.3 Hz, 1H), 4.13-4.02 (m, 1H), 3.81 (d, J=13.0 Hz, 6H), 3.64-3.35 (m, 3H), 3.27-3.02 (m, 2H), 3.02-2.74 (m, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 28% B in 10 min; Wave Length: 254 nm/220 nm; RT1(min): 8.6.

### Synthetic Procedure of V187878

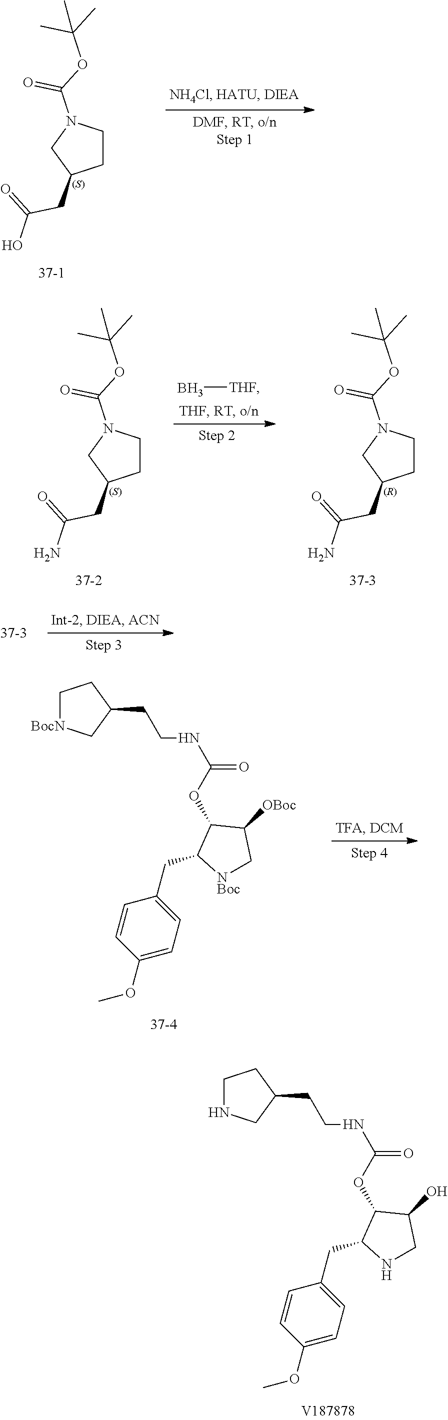

Step 1: To a stirred solution of [(3S)-1-(tert-butoxycarbonyl)pyrrolidin-3-yl]acetic acid (37-1, 500 mg, 2.18 mmol, 1.0 eq.) and NH₄Cl (583.3 mg, 10.91 mmol, 5.0 eq.) in DMF (10 mL) was added HATU (1658.4 mg, 4.36 mmol, 2.0 eq.) and DIEA (1127.4 mg, 8.72 mmol, 4.0 eq.) in portions at room temperature. The resulting mixture was stirred at 25° C. for overnight. The resulting mixture was extracted with EtOAc (3×10 mL). The combined organic layers were washed with water (3×10 mL), dried over anhydrous Na2SO4. After filtration, the filtrate was concentrated under reduced pressure. The residue was purified by reversed-phase flash chromatography to afford tert-butyl (3S)-3-(carbamoylmethyl)pyrrolidine-1-carboxylate (400 mg, 80.3%) as a light yellow oil. MS: m/z: Calc'd for C₁₁H₂₀N₂O₃ [M+H+22]⁺251. Found, 251.

Step 2: To a stirred solution of tert-butyl (3S)-3-(carbamoylmethyl)pyrrolidine-1-carboxylate (37-2, 150 mg, 0.66 mmol, 1.0 eq.) in THF (5 mL) was added BH₃-THF (3.29 mL, 3.285 mmol, 5.0 eq.) in portions at −78° C. The resulting mixture was stirred at −78° C. for 1 h under a nitrogen atmosphere. Desired product could be detected by LCMS. The reaction was quenched with MeOH at 0° C. The resulting mixture was concentrated under reduced pressure. The resulting mixture was used in the next step directly without further purification. MS: m/z: Calc'd for C₁₁H₂₂N₂O₂ [M+H]⁺215. Found, 215.

Step 3 & 4: The title compound was prepared in 35.8% overall yield as a yellow semi-solid according to Substitution reaction for synthesis of Carbamate; General Procedure II using tert-butyl (3R)-3-(2-aminoethyl)pyrrolidine-1-carboxylate (35-3) in STEP 3; Boc Deprotection; General Procedure III in STEP 4. MS: m/z Calc'd for C₁₉H₂₉N₃O₄ [M+H]⁺364. Found, 364. ¹H NMR (400 MHz, Methanol-d₄) δ 7.28-7.21 (m, 2H), 6.96-6.90 (m, 2H), 4.95 (d, J=3.5 Hz, 1H), 4.38 (d, J=4.1 Hz, 1H), 4.19-4.15 (m, J=7.8, 3.5 Hz, 1H), 3.80 (s, 3H), 3.57 (dd, J=12.6, 4.3 Hz, 1H), 3.50 (dd, J=11.5, 7.6 Hz, 1H), 3.44-3.38 (m, J=12.1, 8.5, 3.9 Hz, 1H), 3.27 (d, J=8.6 Hz, 1H), 3.26-3.17 (m, 3H), 3.11 (dd, J=14.1, 7.1 Hz, 1H), 2.98 (dd, J=14.1, 8.5 Hz, 1H), 2.88 (dd, J=11.6, 9.1 Hz, 1H), 2.44-2.21 (m, 2H), 1.81-1.61 (m, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 8.77.

### Synthetic Procedure of V187919

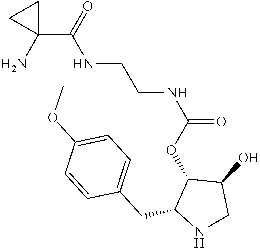 [SMILES: COc1ccc(C[C@H]2NC[C@H](O)[C@H]2OC(=O)NCCNC(=O)C2(N)CC2)cc1]

The title compound was prepared in 63.2% overall yield as a white solid according to Substitution reaction for synthesis of Carbamate; General Procedure II using tert-butyl N-{1-[(2-aminoethyl)carbamoyl]cyclopropyl}carbamate in STEP 1; Boc Deprotection; General Procedure III in STEP 2. MS: m/z Calc'd for C₁₉H₂₈N₄O₅ [M+H]⁺393. found 393. ¹H NMR (400 MHz, Methanol-d₄) δ 7.27-7.21 (m, 2H), 6.96-6.90 (m, 2H), 4.98-4.92 (m, 1H), 4.39 (d, J=4.2 Hz, 1H), 4.19-4.12 (m, 1H), 3.79 (s, 3H), 3.58 (dd, J=12.7, 4.3 Hz, 1H), 3.36 (s, 2H), 3.28-3.22 (m, 2H), 3.19 (s, 1H), 3.11 (dd, J=14.2, 7.0 Hz, 1H), 2.97 (dd, J=14.2, 8.7 Hz, 1H), 1.57-1.43 (m, 2H), 1.43-1.30 (m, 2H). Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 30% B in 20 min; Wave Length: 254 nm/220 nm; RT1(min): 17.13.

### Synthetic Procedure of V187940

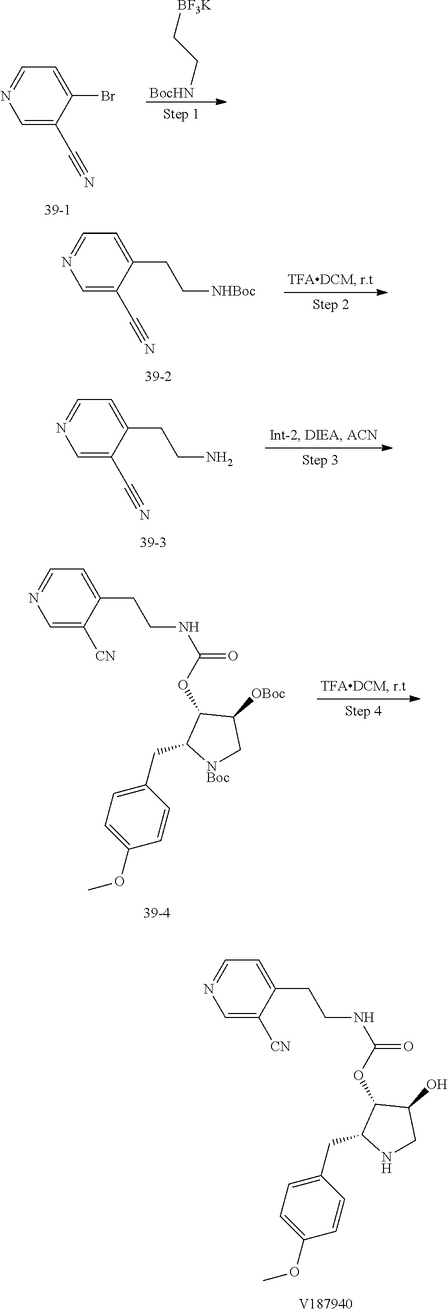

Step 1: To a stirred mixture of 4-bromopyridine-3-carbonitrile (39-1, 500 mg, 2.73 mmol, 1 eq.) and tert-butyl N-[2-(trifluoro-lambda4-boranyl)ethyl]carbamate potassium (1029.0 mg, 4.09 mmol, 1.5 eq.) in Toluene (7.5 mL) and H₂O (2.5 mL) was added Pd(dppf)Cl₂ (399.8 mg, 0.54 mmol, 0.2 eq.) and Cs₂CO₃ (2670.5 mg, 8.19 mmol, 3 eq.) at room temperature under nitrogen atmosphere. The resulting mixture was stirred at 90° C. for overnight under nitrogen atmosphere. The resulting mixture was concentrated under reduced pressure. The residue was dissolved in MeCN (5 mL) and purified by reversed-phase flash chromatography to afford tert-butyl N-[2-(3-cyanopyridin-4-yl)ethyl]carbamate (500 mg, 74.0%) as a yellow solid.

Step 2: To a stirred mixture of tert-butyl N-[2-(3-cyanopyridin-4-yl)ethyl]carbamate (39-2, 230 mg, 0.93 mmol) in DCM (5 mL) was added TFA (1 mL) at room temperature. The resulting mixture was stirred at room temperature for 2 h. The resulting mixture was concentrated under reduced pressure. This resulted in 4-(2-aminoethyl)pyridine-3-carbonitrile (120 mg, 87.6%) as a yellow oil. MS: m/z: Calc'd for C₈H₉N₃[M+H]⁺148. found 148.

Step 3 & 4: The title compound was prepared in 13.5% overall yield as a yellow solid according to Substitution reaction for synthesis of Carbamate; General Procedure II using 4-(2-aminoethyl)pyridine-3-carbonitrile (39-3) in STEP 3; Boc Deprotection; General Procedure III in STEP 4. MS: m/z Calc'd for C₂₁H₂₄N₄O₄ [M+H]⁺397. found 397. ¹H NMR (400 MHz, Methanol-d₄) δ 8.85 (s, 1H), 8.71 (d, J=5.2 Hz, 1H), 7.57 (d, J=5.2 Hz, 1H), 7.27-7.16 (m, 2H), 6.96-6.88 (m, 2H), 4.89 (s, 1H), 4.42-4.36 (m, 1H), 4.13-4.02 (m, 1H), 3.79 (s, 3H), 3.71-3.44 (m, 3H), 3.20 (d, J=12.7 Hz, 1H), 3.16-3.03 (m, 3H), 2.92 (dd, J=14.2, 8.6 Hz, 1H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 30% B in 10 min; Wave Length: 254 nm/220 nm; RT1(min): 8.82.

### Synthetic Procedure of V187948

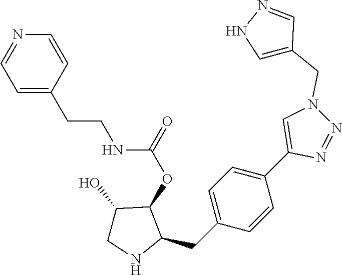 [SMILES: O=C(NCCc1ccncc1)O[C@@H]1[C@@H](O)CN[C@@H]1Cc1ccc(-c2cn(Cc3cn[nH]c3)nn2)cc1]

The title compound was prepared in 30.4% overall yield as a light green solid according to Cyclization reaction for synthesis of Substituted triazoles; General Procedure VI using 4-(azidomethyl)-1H-pyrazole in STEP 1; Boc Deprotection; General Procedure III in STEP 2. MS: m/z Calc'd for C₂₅H₂₈N₈O₃[M+H]⁺489. found 489. ¹H NMR (400 MHz, Methanol-d₄) δ 8.68 (d, J=94.4 Hz, 2H), 8.32 (s, 1H), 8.02 (s, 2H), 7.81 (d, J=8.0 Hz, 4H), 7.36 (d, J=8.0 Hz, 2H), 5.61 (s, 2H), 5.05 (s, 1H), 4.35 (d, J=4.1 Hz, 1H), 4.25-4.16 (m, 1H), 3.67-3.51 (m, 3H), 3.26-3.10 (m, 4H), 3.04 (dd, J=14.1, 8.4 Hz, 1H). Prep-HPLC purification conditions: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 8.77.

### Synthetic Procedure of V187950

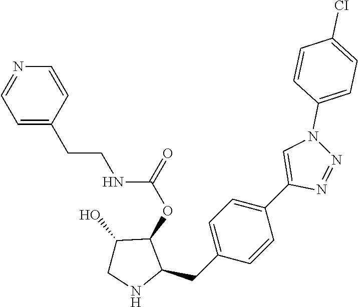

The title compound was prepared in 36.9% overall yield as a white solid according to Cyclization reaction for synthesis of Substituted triazoles; General Procedure VI using 1-azido-4-chlorobenzene in STEP 1; Boc Deprotection; General Procedure III in STEP 2. MS: m/z C₂₇H₂₇ClN₆O₃[M+H]⁺519. found 519. ¹H NMR (400 MHz, Methanol-d₄) δ 8.68-8.97 (s, 1H), 8.69 (s, 2H), 7.98-7.92 (m, 4H), 7.85 (s, 2H), 7.65 (dd, J=8.7, 1.6 Hz, 2H), 7.43 (d, J=8.1 Hz, 2H), 4.96 (s, 1H), 4.37 (d, J=4.0 Hz, 1H), 4.26 (s, 1H), 3.65-3.54 (m, 3H), 3.24 (d, J=13.1 Hz, 1H), 3.18-3.02 (m, 4H). Prep-HPLC purification conditions: Xselect CSH Prep C18 Column, 30*250 mm, 10 μm; Mobile Phase A: Water (0.05% TFA), Mobile Phase B: ACN; Flow rate: 60 mL/min; Gradient: 11% B to 41% B in 10 min; Wave Length: 254 nm/220 nm; RT1(min): 8.75.

### Synthetic Procedure of V187979

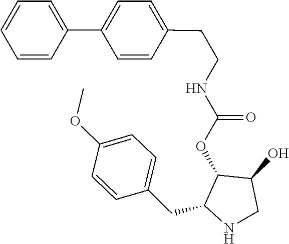 [SMILES: COc1ccc(C[C@H]2NC[C@H](O)[C@H]2OC(=O)NCCc2ccc(-c3ccccc3)cc2)cc1]

The title compound was prepared in 50.3% overall yield as a white solid according to Substitution reaction for synthesis of Carbamate; General Procedure II using 2-{[1,1′-biphenyl]-4-yl}ethanamine in STEP 1; Boc Deprotection; General Procedure III in STEP 2. MS: m/z Calc'd for C₂₇H₃₀N₂O₄[M+H]⁺447. found 447. ¹H NMR (400 MHz, Methanol-d₄) δ 7.60-7.51 (m, 4H), 7.41 (dd, J=8.5, 6.8 Hz, 2H), 7.38-7.29 (m, 3H), 7.19-7.14 (m, 2H), 6.91-6.84 (m, 2H), 4.93 (d, J=3.4 Hz, 1H), 4.36-4.30 (m, 1H), 4.15-4.06 (m, 1H), 3.75 (d, J=0.7 Hz, 3H), 3.59-3.39 (m, 3H), 3.18 (d, J=12.6 Hz, 1H), 3.03 (dd, J=14.2, 7.0 Hz, 1H), 2.99-2.81 (m, 3H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 m/min; Gradient: 20% B to 50% B in 10 min; Wave Length: 254 nm/220 nm; RT1(min): 8.2.

### Synthetic Procedure of V187987

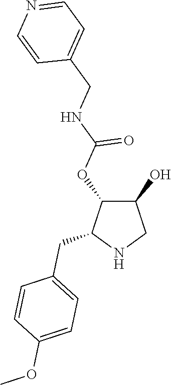 [SMILES: COc1ccc(C[C@H]2NC[C@H](O)[C@H]2OC(=O)NCc2ccncc2)cc1]

The title compound was prepared in 30.8% overall yield as a yellow solid according to Substitution reaction for synthesis of Carbamate; General Procedure II using 4-pyridylmethanamine in STEP 1; Boc Deprotection; General Procedure III in STEP 2. MS: m/z Calc'd for C₁₉H₂₃N₃O₄ [M+H]⁺358. found 358. ¹H NMR (400 MHz, Methanol-d₄) δ 8.78 (s, 2H), 7.93 (s, 2H), 7.28-7.21 (m, 2H), 6.92 (d, J=8.5 Hz, 2H), 4.87 (s, 2H), 4.63 (s, 1H), 4.46-4.40 (m, 1H), 4.21-4.10 (m, 1H), 3.79 (d, J=2.9 Hz, 3H), 3.62-3.50 (m, 1H), 3.24 (d, J=12.6 Hz, 1H), 3.14 (dd, J=14.2, 7.3 Hz, 1H), 3.08-2.98 (m, 1H). Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 7.53.

### Synthetic Procedure of V187988

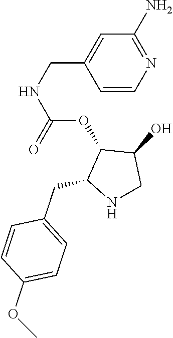 [SMILES: COc1ccc(C[C@H]2NC[C@H](O)[C@H]2OC(=O)NCc2ccnc(N)c2)cc1]

The title compound was prepared in 51.8% overall yield as a white solid according to Substitution reaction for synthesis of Carbamate; General Procedure II using tert-butyl N-[4-(aminomethyl)pyridin-2-yl]carbamate in STEP 1; Boc Deprotection; General Procedure III in STEP 2. MS: m/z Calc'd for C₁₉H₂₄N₄O₄ [M+H]⁺373. found 373. ¹H NMR (400 MHz, Methanol-d₄) δ 7.83 (d, J=7.0 Hz, 1H), 7.25 (d, J=8.5 Hz, 2H), 6.92 (d, J=8.3 Hz, 3H), 6.85 (d, J=6.7 Hz, 1H), 5.00 (s, 1H), 4.40 (d, J=15.7 Hz, 3H), 4.21 (s, 1H), 3.80 (s, 3H), 3.60 (d, J=11.8 Hz, 1H), 3.25 (d, J=12.8 Hz, 1H), 3.19-3.09 (m, 1H), 3.06-2.95 (m, 1H). Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 30% B in 20 min; Wave Length: 254 nm/220 nm; RT1(min): 18.87.

### Synthetic Procedure of V188002

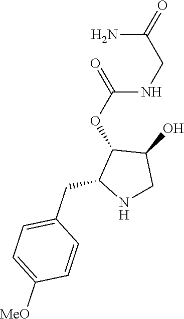 [SMILES: COc1ccc(C[C@H]2NC[C@H](O)[C@H]2OC(=O)NCC(N)=O)cc1]

The title compound was prepared in 12.0% overall yield as a white solid according to Substitution reaction for synthesis of Carbamate; General Procedure II using glycinamide in STEP 1; Boc Deprotection; General Procedure III in STEP 2. MS: m/z Calc'd for C₁₅H₂₁N₃O₅ [M+H]⁺324. Found, 324. ¹H NMR (400 MHz, Methanol-d₄) δ 7.30-7.22 (m, 2H), 6.98-6.90 (m, 2H), 4.96 (d, J=2.9 Hz, 1H), 4.44 (d, J=4.4 Hz, 1H), 4.20-4.11 (m, 1H), 3.85 (d, J=2.9 Hz, 2H), 3.80 (s, 3H), 3.62 (dd, J=12.6, 4.4 Hz, 1H), 3.22 (d, J=12.6 Hz, 1H), 3.15 (dd, J=14.2, 7.1 Hz, 1H), 2.99 (dd, J=14.2, 8.6 Hz, 1H). Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 8.25.

### Synthetic Procedure of V188053

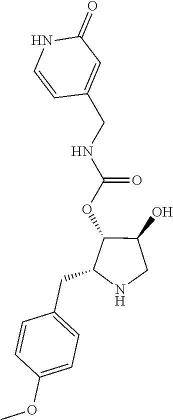 [SMILES: COc1ccc(C[C@H]2NC[C@H](O)[C@H]2OC(=O)NCc2cc[nH]c(=O)c2)cc1]

The title compound was prepared in 51.3% overall yield as a light yellow solid according to Substitution reaction for synthesis of Carbamate; General Procedure II using 4-(aminomethyl)-1H-pyridin-2-one in STEP 1; Boc Deprotection; General Procedure III in STEP 2. MS: m/z Calc'd for C₁₉H₂₃N₃O₅ [M+H]⁺374. Found, 374. ¹H NMR (400 MHz, DMSO-d₆) δ 11.46 (s, 1H), 9.46 (d, J=44.9 Hz, 1H), 9.11 (s, 1H), 7.92-7.72 (m, J=69.5, 6.3 Hz, 1H), 7.36-7.32 (m, J=7.3 Hz, 1H), 7.28-7.03 (m, 2H), 6.90 (dd, J=28.5, 8.4 Hz, 2H), 6.23 (d, J=26.1 Hz, 1H), 6.16-6.06 (m, 1H), 5.98 (s, 1H), 4.76-4.62 (m, 1H), 4.25 (s, 1H), 4.21-3.88 (m, 3H), 3.74 (d, J=4.0 Hz, 3H), 3.10 (d, J=14.5 Hz, 1H), 3.04-2.70 (m, 2H). Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 26% B in 10 min; Wave Length: 254 nm/220 nm; RT1(min): 8.42.

### Synthetic Procedure of V188058

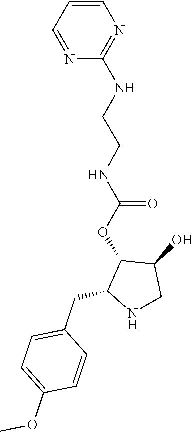 [SMILES: COc1ccc(C[C@H]2NC[C@H](O)[C@H]2OC(=O)NCCNc2ncccn2)cc1]

The title compound was prepared in 27.0% overall yield as a yellow oil according to Substitution reaction for synthesis of Carbamate; General Procedure II using N1-(pyrimidin-2-yl)ethane-1,2-diamine in STEP 1; Boc Deprotection; General Procedure III in STEP 2. MS: m/z Calc'd for C₁₉H₂₅N₅O₄ [M+H]⁺388. found 388. ¹H NMR (400 MHz, Methanol-d₄) δ 8.45 (s, 2H), 7.21 (d, J=8.3 Hz, 2H), 6.90 (d, J=8.2 Hz, 2H), 6.86-6.77 (m, 1H), 4.92 (d, J=3.5 Hz, 1H), 4.36 (d, J=4.2 Hz, 1H), 4.13-4.02 (m, 1H), 3.78 (s, 3H), 3.71-3.60 (m, 2H), 3.55 (dd, J=12.7, 4.3 Hz, 1H), 3.43-3.31 (m, 2H), 3.19 (d, J=12.6 Hz, 1H), 3.07 (dd, J=14.1, 7.1 Hz, 1H), 2.94 (dd, J=14.1, 8.4 Hz, 1H). Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.97.

### Synthetic Procedure of V188106

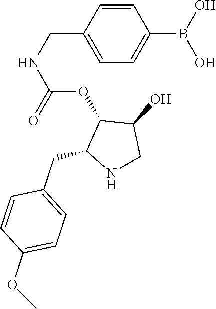 [SMILES: COc1ccc(C[C@H]2NC[C@H](O)[C@H]2OC(=O)NCc2ccc(B(O)O)cc2)cc1]

The title compound was prepared in 45.1% overall yield as a white solid according to Substitution reaction for synthesis of Carbamate; General Procedure II using 4-(aminomethyl) phenylboronic acid in STEP 1; Boc Deprotection; General Procedure III in STEP 2. MS: m/z Calc'd for C₂₀H₂₅BN₂O₆ [M+H]⁺401. found 401. ¹H NMR (400 MHz, Methanol-d₄) δ 7.78 (d, J=7.7 Hz, 1H), 7.64 (d, J=7.6 Hz, 1H), 7.34 (dd, J=13.7, 7.6 Hz, 2H), 7.23-7.16 (m, 2H), 6.87 (dd, J=7.0, 3.7 Hz, 2H), 4.42-4.28 (m, 3H), 4.16 (s, 1H), 3.59 (dd, J=12.6, 4.1 Hz, 1H), 4.32 (s, 4H), 3.23 (d, J=12.6 Hz, 1H), 3.10 (dd, J=14.1, 7.8 Hz, 1H), 2.99 (dd, J=13.4, 8.2 Hz, 1H). Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 4% B to 34% B in 10 min; Wave Length: 254 nm/220 nm; RT1(min): 8.45.

### Synthetic Procedure of V188109

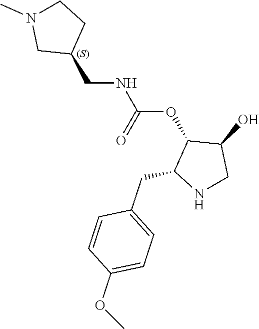

The title compound was prepared in 36.0% overall yield as a light green semi-solid according to Substitution reaction for synthesis of Carbamate; General Procedure II using 1-[(3S)-1-methylpyrrolidin-3-yl]methanamine in STEP 1; Boc Deprotection; General Procedure III in STEP 2. MS: m/z Calc'd for C₁₉H₂₉N₃O₄ [M+H]⁺364. found 364. ¹H NMR (400 MHz, Methanol-d₄) δ 7.27-7.20 (m, 2H), 6.97-6.89 (m, 2H), 4.96 (d, J=3.5 Hz, 1H), 4.39 (d, J=4.2 Hz, 1H), 4.22-4.12 (m, 1H), 3.80 (s, 4H), 3.64-3.55 (m, 1H), 3.48 (d, J=11.9 Hz, 1H), 3.31-3.23 (m, 3H), 3.21-3.08 (m, 2H), 3.05-2.98 (m, 1H), 2.96 (s, 3H), 2.89-2.78 (m, 1H), 2.68 (s, 1H), 2.28 (d, J=48.1 Hz, 1H), 1.89 (d, J=62.0 Hz, 1H). Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min mL/min; Gradient: 2% B to 23% B in 10 min; Wave Length: 254 nm/220 nm; RT1(min): 7.65.

### Synthetic Procedure of V187942

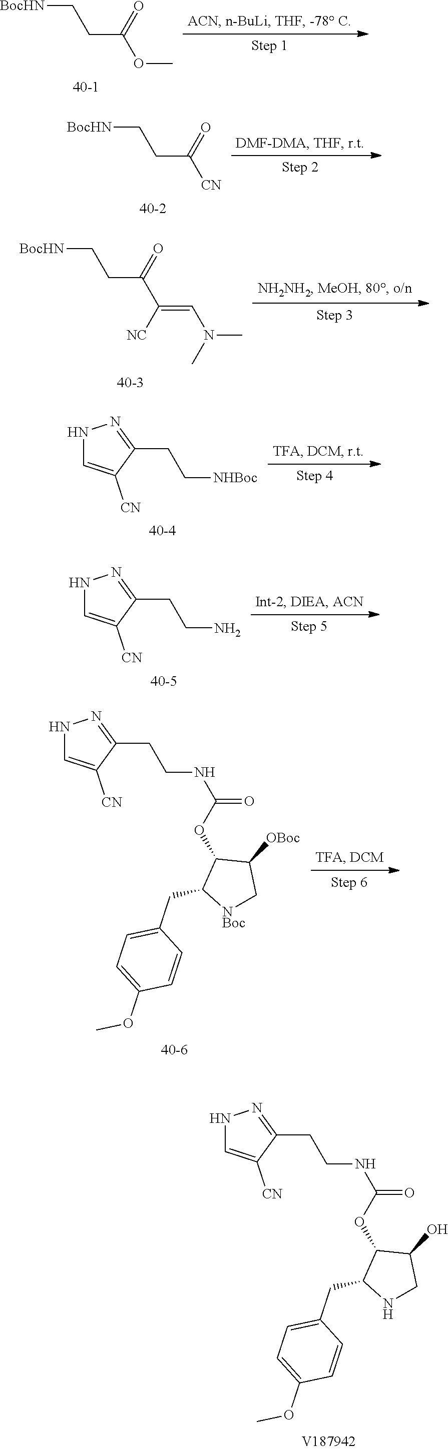

Step 1: A solution of n-BuLi (0.79 g, 12.3 mmol, 2.5 equiv) in THF (15 mL) was treated with ACN (0.50 g, 12.3 mmol, 2.5 equiv) for 1 h at −78° C. under nitrogen atmosphere followed by the addition of methyl 3-[(tert-butoxycarbonyl)amino]propanoate (40-1, 1 g, 4.92 mmol, 1 equiv) at −78° C. The resulting mixture was stirred at −50° C. for 1 h under nitrogen atmosphere. The reaction was quenched by the addition of sat. NH₄Cl (aq.) (10 mL) at 0° C. The resulting mixture was quenched with water and extracted with EA. The extracts were washed with brine, dried over anhydrous sodium sulfate, filtered and concentrated. The residue was purified by silica gel column chromatography, eluted with PE/EA (3:1) to afford tert-butyl N-(4-cyano-3-oxobutyl)carbamate (500 mg, 47.8%) as a brown oil.

Step 2: To a stirred mixture of tert-butyl N-[2-(3-cyanopyridin-4-yl)ethyl]carbamate (40-2, 230 mg, 0.93 mmol) in DCM (5 mL) was added TFA (1 mL) at room temperature. The resulting mixture was stirred at room temperature for 2 h. The resulting mixture was concentrated under reduced pressure. This resulted in 4-(2-aminoethyl)pyridine-3-carbonitrile (120 mg, 87.6%) as a yellow oil. MS: m/z: Calc'd for C₈H₉N₃[M+H]⁺148. found 148.

Step 3: To a stirred solution/mixture of tert-butyl N-[(4E)-4-cyano-4-[(dimethylamino)methylidene]-3-oxobutyl]carbamate (40-3, 300 mg, 1.12 mmol, 1 eq.) in MeOH (5 mL) was added hydrazine hydrate (168.5 mg, 3.36 mmol, 3 eq.) at room temperature under nitrogen atmosphere. The resulting mixture was stirred at 80° C. for overnight under nitrogen atmosphere. The resulting mixture was concentrated under reduced pressure. The residue was dissolved in DMSO (5 mL). The residue was purified by reversed-phase flash chromatography to afford tert-butyl N-[2-(4-cyano-1H-pyrazol-3-yl)ethyl]carbamate (200 mg, 75.4%) as a yellow oil. MS: m/z: Calc'd for C₁₁H₁₆N₄O₂ [M+H+22]⁺259. found 259.

Step 4: To a stirred mixture of tert-butyl N-[2-(4-cyano-1H-pyrazol-3-yl)ethyl]carbamate (40-4, 300 mg, 1.27 mmol) in DCM (5 mL) was added TFA (1 mL, 13.46 mmol, 10.6 eq) at room temperature. The resulting mixture was stirred at room temperature for 3 h. The resulting mixture was concentrated under vacuum. This resulted in 3-(2-aminoethyl)-1H-pyrazole-4-carbonitrile (160 mg, 92.5%) as a yellow oil. MS: m/z: Calc'd for C₆H₈N₄ [M+H]⁺137. found 137.

Step 5 & 6: The title compound was prepared in 24.5% overall yield as a white solid according to Substitution reaction for synthesis of Carbamate; General Procedure II using 3-(2-aminoethyl)-1H-pyrazole-4-carbonitrile (40-5) in STEP 5; Boc Deprotection; General Procedure III in STEP 6. MS: m/z Calc'd for C₁₉H₂₃N₅O₄[M+H]⁺386. found 386. ¹H NMR (400 MHz, Methanol-d₄) δ 8.07 (s, 1H), 7.21 (d, J=8.5 Hz, 2H), 6.96-6.88 (m, 2H), 4.90 (s, 1H), 4.41 (d, J=4.2 Hz, 1H), 4.13-4.02 (m, 1H), 3.79 (s, 3H), 3.69-3.50 (m, 2H), 3.45-3.32 (m, 1H), 3.20 (d, J=12.6 Hz, 1H), 3.14-2.91 (m, 4H). Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 32% B in 10 min; Wave Length: 254 nm/220 nm; RT1(min): 8.83.

### Synthetic Procedure of V188003

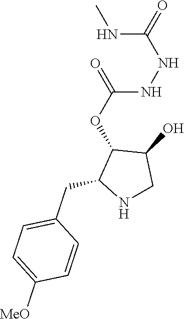 [SMILES: CNC(=O)NNC(=O)O[C@@H]1[C@@H](O)CN[C@@H]1Cc1ccc(OC)cc1]

The title compound was prepared in 42.5% overall yield as a white solid according to Substitution reaction for synthesis of Carbamate; General Procedure II using 1-amino-3-methylurea in STEP 1; Boc Deprotection; General Procedure III in STEP 2. MS: m/z Calc'd for C₁₅H₂₂N₄O₅ [M+H]⁺339. Found, 339. ¹H NMR (400 MHz, Methanol-d₄) δ 7.26 (d, J=8.5 Hz, 2H), 6.99-6.90 (m, 2H), 5.00 (d, J=3.3 Hz, 1H), 4.46 (s, 1H), 4.20-4.14 (m, J=10.4, 7.2, 3.3 Hz, 1H), 3.80 (s, 3H), 3.63-3.49 (m, 1H), 3.23 (d, J=12.6 Hz, 1H), 3.16-3.03 (m, 1H), 2.96 (d, J=17.9 Hz, 1H), 2.77 (s, 3H). Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 8.74.

### Synthetic Procedure of V188018

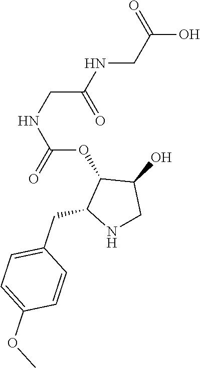 [SMILES: COc1ccc(C[C@H]2NC[C@H](O)[C@H]2OC(=O)NCC(=O)NCC(=O)O)cc1]

The title compound was prepared in 45.0% overall yield as a light orange solid according to Substitution reaction for synthesis of Carbamate; General Procedure II using tert-butyl 2-(2-aminoacetamido)acetate in STEP 1; Boc Deprotection; General Procedure III in STEP 2. MS: m/z Calc'd for C₃₁H₄₇N₃O₁₁ [M+H]⁺364. found 364. ¹H NMR (400 MHz, Methanol-d₄) δ 7.26 (dd, J=15.7, 8.2 Hz, 2H), 6.92 (d, J=8.3 Hz, 2H), 4.59 (s, 1H), 4.42 (d, J=4.1 Hz, 1H), 4.05 (s, 1H), 3.96-3.83 (m, 2H), 3.79 (s, 4H), 3.76-3.65 (m, 1H), 3.60-3.51 (m, 1H), 3.17-3.06 (m, 3H). Prep-HPLC purification conditions: Column: Column: Welch Ultimate AQ-C18 50*250, 10 μm; Mobile Phase A: Water (10nmol/LNH₄HCO₃), Mobile Phase B: ACN; Flow rate: 60 mL/min; Gradient: 0% B to 40% B in 20 min; Wave Length: 254 nm/220 nm; RT1(min): 41.32.

### Example 3: Synthesis of Intermediates to Additional Compounds of the Present Disclosure

### Synthetic Procedure of Intermediates (Int-1-13)

### Synthetic Procedure of Intermediates Int-1 and Int-2

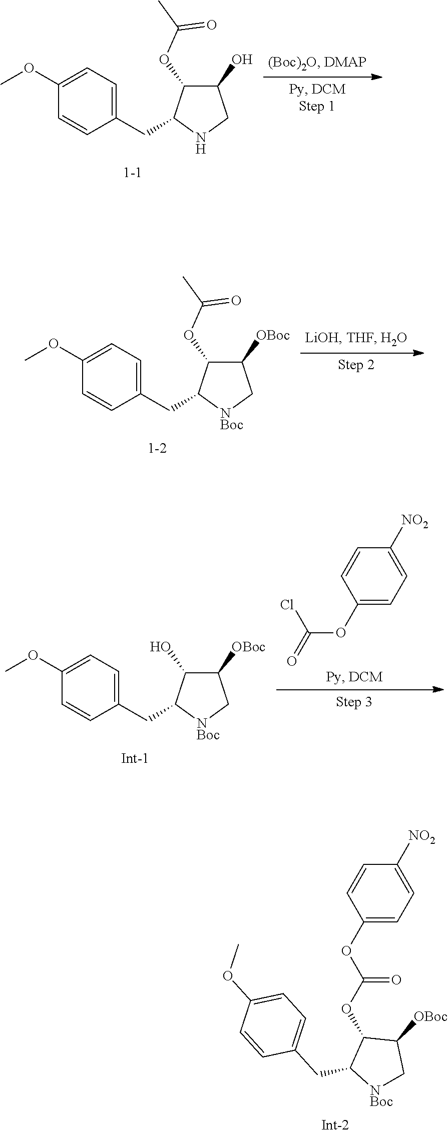

Step 1: To a stirred solution of anisomycin (5 g, 18.85 mmol, 1 eq.) in Pyridine (100 mL) was added Boc₂O (41.13 g, 188.46 mmol, 10 eq.) and DMAP (3.45 g, 28.27 mmol, 1.5 eq.) at room temperature. The resulting mixture was stirred over night at 25° C. Upon completion, the reaction mixture was concentrated in vacuo. The residue was purified by reversed-phase flash chromatography (0.05% NH₄HCO₃ in H₂O/ACN) to afford tert-butyl (2R,3S,4S)-3-(acetyloxy)-4-[(tert-butoxycarbonyl)oxy]-2-[(4-methoxyphenyl)methyl]pyrrolidine-1-carboxylate (8 g, 91.2%) as a light yellow oil. MS: m/z: Calc'd for C₂₄H₃₅NO₈ [M+H−56−100]⁺310. found 310.

Step 2: To a stirred solution of tert-butyl (2R,3S,4S)-3-(acetyloxy)-4-[(tert-butoxycarbonyl)oxy]-2-[(4-methoxyphenyl)methyl]pyrrolidine-1-carboxylate (8 g, 17.18 mmol, 1 eq.) in THF (100 mL) was added LiOH (1.23 g, 51.55 mmol, 3 eq.) and H₂O (10 mL) at room temperature. The resulting mixture was stirred over night at 25° C. The resulting mixture was extracted with EtOAc (3×30 mL). The combined organic layers were washed with water (3×10 mL), dried over anhydrous Na₂SO₄. After filtration, the filtrate was concentrated under reduced pressure to afford crude product tert-butyl (2R,3S,4S)-4-[(tert-butoxycarbonyl)oxy]-3-hydroxy-2-[(4-methoxyphenyl)methyl]pyrrolidine-1-carboxylate (7.2 g, 98.9%) as a light yellow solid, which was used directly in the next step without further purification. MS: m/z: Calc'd for C₂₂H₃₃NO₇ [M+H−56−56]⁺312. found 312.

### Step 3:

To a stirred solution of tert-butyl (2R,3S,4S)-4-[(tert-butoxycarbonyl)oxy]-3-hydroxy-2-[(4-methoxyphenyl)methyl]pyrrolidine-1-carboxylate (4.7 g, 11.10 mmol, 1 eq.) and 4-nitrophenyl carbonochloridate (3.36 g, 16.67 mmol, 1.5 eq.) in DCM (50 mL) was added Pyridine (1.76 g, 22.25 mmol, 2 eq.) at room temperature. The resulting mixture was stirred at room temperature for another 2 h. After completion of reaction monitored by LCMS. The mixture was concentrated, the residue was purified by silica gel column chromatography, eluted with PE/EA (5:1) to afford tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (5.4 g, 82.7%) as a light yellow oil. MS: m/z: Calc'd for C₂₉H₃₆N₂O₁₁ [M+H−100]⁺489. found 489.

### Synthetic Procedure of Intermediates Int-3 and Int-4

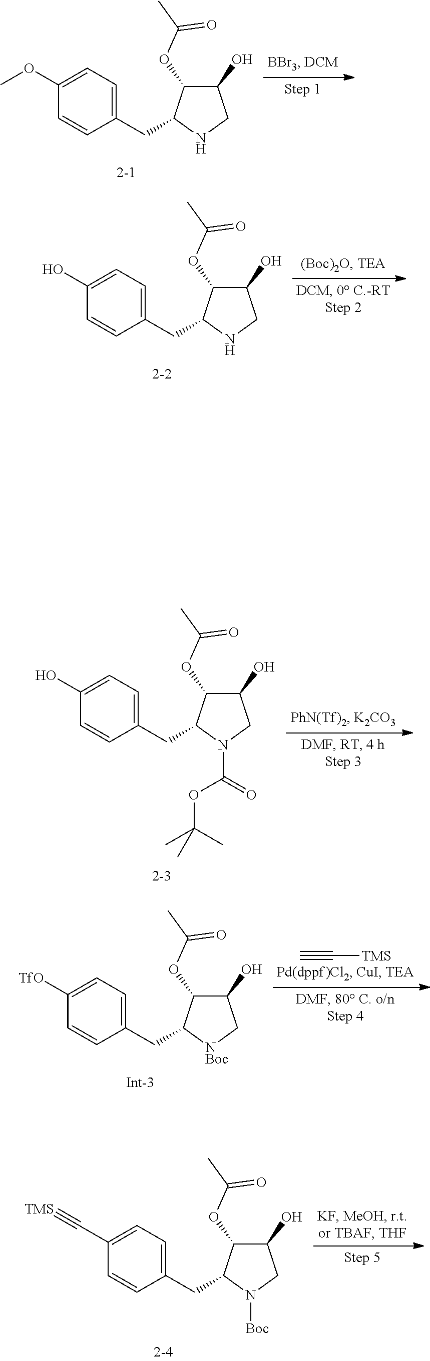

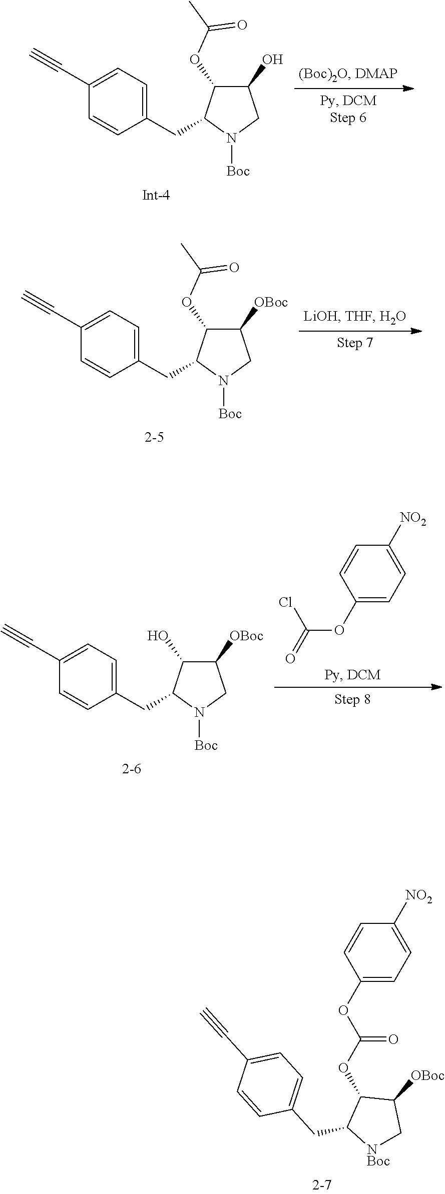

Step 1: To a stirred solution of anisomycin (1.5 g, 5.65 mmol, 1 equiv) in Dichloromethane (8 mL) was added Boron tribromide (16.8 mL, 3 equiv) at −78° C. The reaction mixture was stirred at −78° C. for 2 h and warmed to room temperature. The mixture was stirred for 1 h at room temperature and quenched by saturated NaHCO₃ solution. The DCM was removed, and the solution was lyophilized to obtain (2R,3S,4S)-4-hydroxy-2-[(4-hydroxyphenyl)methyl]pyrrolidin-3-yl acetate (1.8 g, 85.41%) as a crude. MS: m/z: Calc'd for C₁₃H₁₇NO₄ [M+H]⁺252. found 252.

Step 2: To a stirred solution of (2R,3S,4S)-4-hydroxy-2-[(4-hydroxyphenyl)methyl]pyrrolidin-3-yl acetate (1.8 g, 7.16 mmol, 1 equiv) and triethylamine (2.54 g, 25.07 mmol, 3.5 equiv) in DCM (30 mL) was added di-tert-butyl dicarbonate (1.88 g, 8.59 mmol, 1.2 equiv) at 0° C. The mixture was stirred at room temperature for 3 h. After completion of the reaction monitored by LCMS, the reaction mixture was filtrated. The filtrate was concentrated. The residue was purified by a reversed-phase column to obtain tert-butyl (2R,3S,4S)-3-(acetyloxy)-4-hydroxy-2-[(4-hydroxyphenyl)methyl]pyrrolidine-1-carboxylate (1.7 g, 67.54%) as a white solid. MS: m/z: Calc'd for C₁₈H₂₅NO₆ [M−H]⁻ 350. found 350.

Step 3: To a stirred solution of tert-butyl (2R,3S,4S)-3-(acetyloxy)-4-hydroxy-2-[(4-hydroxyphenyl)methyl]pyrrolidine-1-carboxylate (1.7 g, 4.83 mmol,) and Potassium carbonate (2.01 g, 14.51 mmol, 3 equiv) in DMF (16 mL) was added 1,1,1-trifluoro-N-phenyl-N-trifluoromethanesulfonylmethanesulfonamide (2.25 g, 6.28 mmol, 1.3 equiv) at 0° C. The reaction mixture was stirred at room temperature for 2 h. After completion of the reaction monitored by LCMS, the reaction mixture was filtrated. The filtrate was injected into a reversed-phase column and purified to obtain tert-butyl (2R,3S,4S)-3-(acetyloxy)-4-hydroxy-2-{[4-(trifluoromethanesulfonyloxy)phenyl]methyl}pyrrolidine-1-carboxylate (1.5 g, 64.13%) as a white solid. MS: m/z: Calc'd for C₁₉H₂₄F₃NO₈S [M+NH₄]⁺501. found 501.

Step 4: To a stirred solution of tert-butyl (2R,3S,4S)-3-(acetyloxy)-4-hydroxy-2-{[4-(trifluoromethanesulfonyloxy)phenyl]methyl}pyrrolidine-1-carboxylate (200 mg, 0.42 mmol, 1 equiv) and trimethylsilylacetylene (121.9 mg, 1.24 mmol, 3 equiv) in DMF (10 mL) was added TEA (167.4 mg, 1.65 mmol, 4 equiv), CuI (7.9 mg, 0.04 mmol, 0.1 equiv) and Pd(dppf)Cl₂·CH₂Cl₂ (50.6 mg, 0.06 mmol, 0.15 equiv) in portions at room. The reaction was placed under vacuum, sonicated and backfilled with nitrogen. The solution was stirred at 80° C. for 12 h. Desired product could be detected by LCMS. Water was used to quench the reaction, extracted with EA, concentrated, the residue was purified by Prep-TLC (PE/EA 2:1) to afford tert-butyl (2R,3S,4S)-3-(acetyloxy)-4-hydroxy-2-({4-[2-(trimethylsilyl)ethynyl]phenyl}methyl)pyrrolidine-1-carboxylate (160 mg, 89.6%) as a yellow oil. MS: m/z: Calc'd for C₂₃H₃₃NO₅Si [M-100]⁺ 332. found 332.

Step 5: A solution of tert-butyl (2R,3S,4S)-3-(acetyloxy)-4-hydroxy-2-({4-[2-(trimethylsilyl)ethynyl]phenyl}methyl)pyrrolidine-1-carboxylate (170 mg, 0.39 mmol, 1 equiv) and Triethylamine trihydrofluoride (190.5 mg, 1.18 mmol, 3.0 equiv) in THF (5 mL) was stirred at 60° C. for 12 h. Desired product could be detected by LCMS. Concentrated, the residue was purified by Prep-TLC (PE/EA 3:1) to afford tert-butyl (2R,3S,4S)-3-(acetyloxy)-2-[(4-ethynylphenyl)methyl]-4-hydroxypyrrolidine-1-carboxylate (135 mg, 95.36%) as a light yellow solid. MS: m/z: Calc'd for C₂₀H₂₅NO₅ [M-56]⁺304. found 304.

### Synthetic Procedure of Intermediates Int-5, Int-6, Int-6, and Int-8

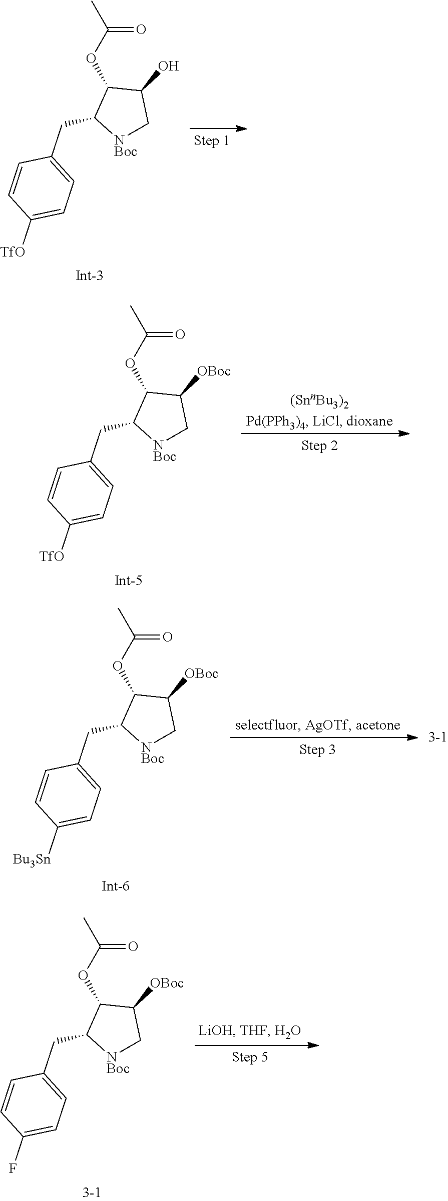

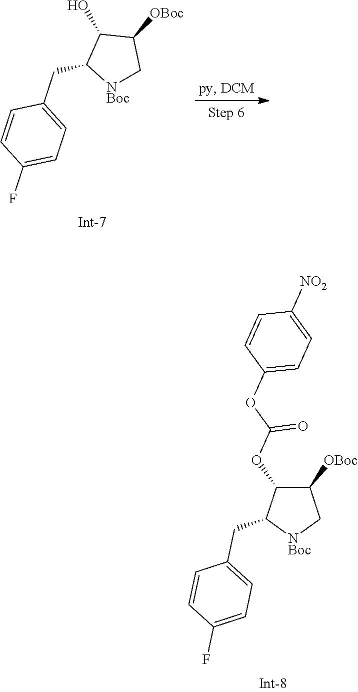

Step 1: To a stirred mixture of tert-butyl (2R,3S,4S)-3-(acetyloxy)-4-hydroxy-2-{[4-(trifluoromethanesulfonyloxy)phenyl]methyl}pyrrolidine-1-carboxylate (Int-3, 1000 mg, 2.06 mmol, 1 equiv) and DMAP (505.4 mg, 4.13 mmol, 2 equiv) in Py (10 mL) was added di-tert-butyl dicarbonate (4514.3 mg, 20.68 mmol, 10 equiv) at room temperature. The resulting mixture was stirred at room temperature for overnight. The resulting mixture was concentrated under reduced pressure. The residue was dissolved in MeCN (5 mL). The residue was purified by reversed-phase flash chromatography to afford tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (1000 mg, 82.8% yield, 95% purity) as a yellow solid. MS: m/z: Calc'd for C₂₄H₃₂F₃NO₁₀S [M+22]⁺ 606. found 606.

Step 2: To a solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (Int-5, 40 mg, 0.069 mmol, 1 equiv) and hexabutyldistannane (80 mg, 0.138 mmol, 2 equiv) in dioxane (2 mL) was added LiCl (15 mg, 0.35 mmol, 5 equiv) and Pd(PPh₃)₄(8 mg, 0.007 mmol, 0.1 equiv). The reaction was placed under vacuum, sonicated and backfilled with nitrogen. The mixture was stirred at 100° C. for overnight. Desired product could be detected by LCMS. The mixture was diluted with EA, washed with water, dried and concentrated. The crude product was purified by Prep-TLC with the following conditions (PE:EA=5:1) to afford tert-butyl (2R,3S,4S)-3-(acetyloxy)-4-[(tert-butoxycarbonyl)oxy]-2-{[4-(tributylstannyl)phenyl]methyl}pyrrolidine-1-carboxylate (10 mg, 20.14%) as a colorless oil. MS: m/z: Calc'd for C₃₅H₅₉NO₇ [M+H]⁺726. found 726.

Step 2: To a solution of tert-butyl (2R,3S,4S)-3-(acetyloxy)-4-[(tert-butoxycarbonyl)oxy]-2-{[4-(tributylstannyl)phenyl]methyl}pyrrolidine-1-carboxylate (Int-5, 110 mg, 0.15 mmol, 1 equiv), Silver trifluoromethanesulfonate (4 mg, 0.015 mmol, 0.1 equiv) and NaHCO₃ (26 mg, 0.30 mmol, 2 equiv) in acetone (5 mL) was added Selectfluor (81 mg, 0.23 mmol, 1.5 equiv). The mixture was stirred at 60° C. for 1 hour under N₂. Desired product could be detected by LCMS. The mixture was filtered and the filtrate was concentrated to give the crude product which was used in the next step without further purification. MS: m/z: Calc'd for C₂₃H₃₂FNO₇ [M+H−56]⁺398. found 398.

Step 3: To a solution of tert-butyl (2R,3S,4S)-3-(acetyloxy)-4-[(tert-butoxycarbonyl)oxy]-2-[(4-fluorophenyl)methyl]pyrrolidine-1-carboxylate (3-1, 100 mg, 0.22 mmol, 1 equiv) in THF (5 mL) and H₂O (0.5 mL) was added LiOH (16 mg, 0.66 mmol, 3 equiv). The mixture was stirred at rt for 6 hours. Desired product could be detected by LCMS. The mixture was diluted with EA, washed with water. The organic layer was concentrated to give the crude product which was used in the next step without further purification. MS: m/z: Calc'd for C₂₁H₃₀FNO₆ [M+H]⁺412. found 412.

Step 4: To the solution of tert-butyl (2R,3S,4S)-4-[(tert-butoxycarbonyl)oxy]-2-[(4-fluorophenyl)methyl]-3-hydroxypyrrolidine-1-carboxylate (Int-6, 60 mg, 0.15 mmol, 1 equiv) and 4-nitrophenyl carbonochloridate (44 mg, 0.22 mmol, 1.5 equiv) in DCM (3 mL) was added Pyridine (23 mg, 0.29 mmol, 2 equiv). The mixture was stirred at rt for overnight. After completion of reaction monitored by LCMS. The mixture was concentrated to give the crude product which was purified by reverse phase flash column (C18 silica gel; mobile phase, MeCN in Water (0.1% TFA), 0% to 100% gradient in 25 min; detector, UV 220 nm). The tert-butyl (2R,3S,4S)-4-[(tert-butoxycarbonyl)oxy]-2-[(4-fluorophenyl)methyl]-3-[(4-nitrophenoxycarbonyl)oxy]pyrrolidine-1-carboxylate (50 mg, 59.5%) was obtained as a yellow oil. MS: m/z: Calc'd for C₂₈H₃₃FN₂O₁₀ [M+H]⁺577. found 577.

### Synthetic Procedure of Intermediates Int-9 and Int-10

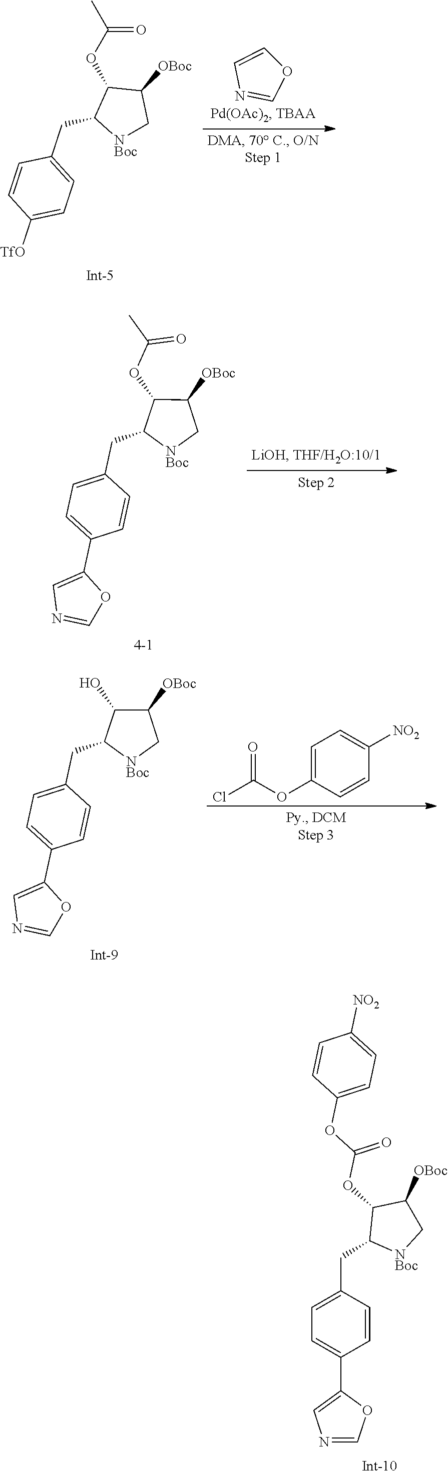

Step 1: To a solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (Int-5, 1000 mg, 1.71 mmol, 1 eq.) and oxazole (177.5 mg, 2.51 mmol, 1.5 eq.) in DMA (10 mL) were added Tetrabutylammonium acetate (774.9 mg, 2.57 mmol, 1.5 eq.) and Pd(OAc)₂ (38.4 mg, 0.17 mmol, 0.1 eq.). The reaction was placed under vacuum, sonicated and backfilled with nitrogen. The mixture was stirred at 70° C. overnight. Desired product could be detected by LCMS. The resulting mixture was filtered, the filter cake was washed with THF (2×2 mL). The filtrate was concentrated under reduced pressure. The residue was purified by reversed-phase flash chromatography (MeCN in Water (5.0 mmol/L NH₄HCO₃)) to afford tert-butyl (2S,3S,4S)-3-(acetyloxy)-4-[(tert-butoxycarbonyl)oxy]-2-{[4-(1,3-oxazol-5-yl)phenyl]methyl}pyrrolidine-1-carboxylate (660 mg, 76.6%) as a light yellow solid. MS: m/z: Calc'd for C₂₆H₃₄N₂O₈ [M+H]⁺503. found 503.

Step 2: To a solution of tert-butyl (2S,3S,4S)-3-(acetyloxy)-4-[(tert-butoxycarbonyl)oxy]-2-{[4-(1,3-oxazol-5-yl)phenyl]methyl}pyrrolidine-1-carboxylate (4-1, 660 mg, 1.31 mmol, 1 eq.) in THF (10 mL) and H₂O (1 mL) was added LiOH (157.2 mg, 6.56 mmol, 5 eq.). The mixture was stirred at rt for 6 hours. Desired product could be detected by LCMS. The resulting mixture was diluted with water (10 mL). The mixture was neutralized to pH 6 with 1M HCl (aq.). The resulting mixture was extracted with EtOAc (3×30 mL). The combined organic layers were dried over anhydrous Na₂SO₄. After filtration, the filtrate was concentrated under reduced pressure. The residue was purified by reversed-phase flash chromatography (MeCN in Water (5.0 mmol/L NH₄HCO₃)) to afford tert-butyl (2S,3S,4S)-4-[(tert-butoxycarbonyl)oxy]-3-hydroxy-2-{[4-(1,3-oxazol-5-yl)phenyl]methyl}pyrrolidine-1-carboxylate (500 mg, 82.7%) as a light yellow oil. MS: m/z: Calc'd for C₂₄H₃₂H₂O₇ [M+H]⁺461. found 461.

Step 3: To the solution of tert-butyl (2S,3S,4S)-4-[(tert-butoxycarbonyl)oxy]-3-hydroxy-2-{[4-(1,3-oxazol-5-yl)phenyl]methyl}pyrrolidine-1-carboxylate (Int-9, 500 mg, 1.08 mmol, 1 eq.) and 4-nitrophenyl carbonochloridate (437.6 mg, 2.17 mmol, 2 eq.) in DCM (5 mL) was added Pyridine (257.6 mg, 3.25 mmol, 3 eq.). The mixture was stirred at rt for overnight. After completion of reaction monitored by LCMS. The mixture was concentrated to give the crude product which was purified by Prep-TLC (PE/EA 3:1) to afford tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (320 mg, 47.1%) as a light yellow solid. MS: m/z: Calc'd for C31H35N3011 [M+H]⁺626. found 626.

### Synthetic Procedure of Intermediate Int-11

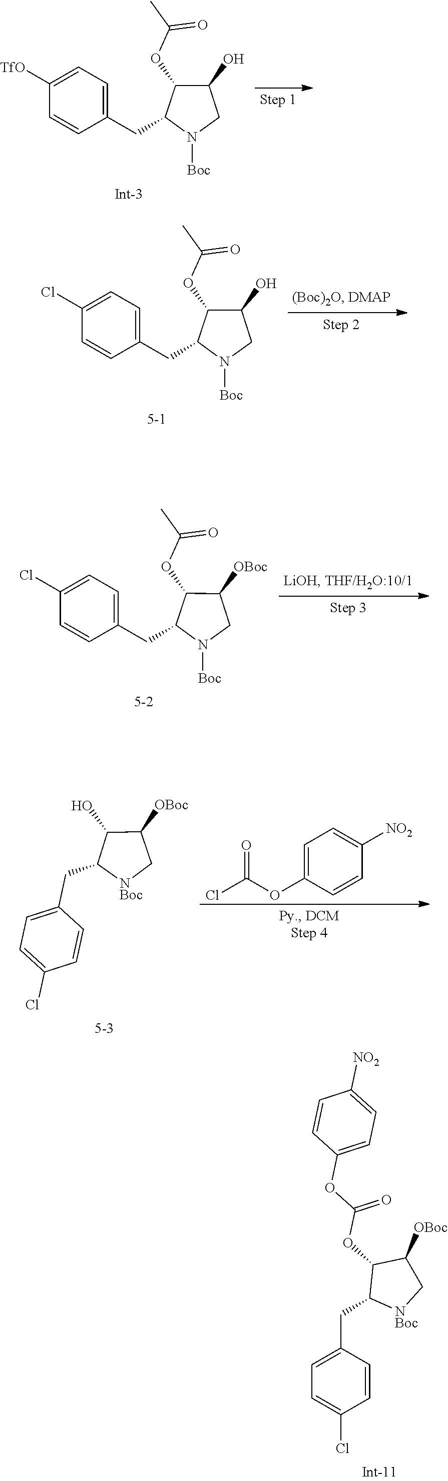

Step 1: To a stirred solution/mixture of tert-butyl (2R,3S,4S)-3-(acetyloxy)-4-hydroxy-2-{[4-(trifluoromethanesulfonyloxy)phenyl]methyl}pyrrolidine-1-carboxylate (Int-3, 800 mg, 1.65 mmol, 1 equiv) di-tert-butyl({3,6-dimethoxy-2-[2,4,6-tris(propan-2-yl)phenyl]phenyl})phosphane (192.4 mg, 0.39 mmol, 0.24 equiv) and potassium chloride (246.7 mg, 3.31 mmol, 2 equiv) in 1,4-dioxane (10 mL) was added KF (48.07 mg, 0.82 mmol, 0.5 equiv) and Pd₂(dba)₃ (75.7 mg, 0.08 mmol, 0.05 equiv) at room temperature under nitrogen atmosphere. The resulting mixture was stirred for overnight at 130° C. under nitrogen atmosphere. The resulting mixture was concentrated under reduced pressure.

The residue was dissolved in MeCN (5 mL). The residue was purified by reversed-phase flash chromatography to afford tert-butyl (2R,3S,4S)-3-(acetyloxy)-2-[(4-chlorophenyl)methyl]-4-hydroxypyrrolidine-1-carboxylate (400 mg, 65.3%) as a yellow oil. MS: m/z: Calc'd for C₁₈H₂₄ClNO₅ [M+22]⁺ 392. found 392.

Step 2: To a stirred mixture of tert-butyl (2R,3S,4S)-3-(acetyloxy)-2-[(4-chlorophenyl)methyl]-4-hydroxypyrrolidine-1-carboxylate (6-1, 350 mg, 0.96 mmol, 1 equiv) and (Boc)₂O (1032.7 mg, 4.73 mmol, 5 equiv) in Pyridine (10 mL) was added DMAP (173.3 mg, 1.41 mmol, 1.5 equiv) at room temperature. The resulting mixture was stirred at room temperature for overnight The resulting mixture was concentrated under reduced pressure. The residue was dissolved in DMSO (5 mL). The residue was purified by reversed-phase flash chromatography to afford tert-butyl (2R,3S,4S)-3-(acetyloxy)-4-[(tert-butoxycarbonyl)oxy]-2-[(4-chlorophenyl)methyl]pyrrolidine-1-carboxylate (400 mg, 89.4%) as a yellow solid. MS: m/z: Calc'd for C₂₃H₃₂ClNO₇ [M-112]⁺358; found 358.

Step 3: To a stirred mixture of tert-butyl (2R,3S,4S)-3-(acetyloxy)-4-[(tert-butoxycarbonyl)oxy]-2-[(4-chlorophenyl)methyl]pyrrolidine-1-carboxylate (390 mg, 0.83 mmol, 1 equiv) in THF (10 mL) and water (1 mL) was added LiOH (59.6 mg, 2.49 mmol, 3 equiv) at room temperature. The resulting mixture was stirred at room temperature for overnight. The resulting mixture was concentrated under reduced pressure. The residue was purified by reversed-phase flash chromatography to afford tert-butyl (2R,3S,4S)-4-[(tert-butoxycarbonyl)oxy]-2-[(4-chlorophenyl)methyl]-3-hydroxypyrrolidine-1-carboxylate (300 mg, 84.4%) as a white solid. MS: m/z: Calc'd for C₂₁H₃₀ClNO₆ [M+22]⁺450. found 450.

Step 4: To a stirred mixture of tert-butyl (2R,3S,4S)-4-[(tert-butoxycarbonyl)oxy]-2-[(4-chlorophenyl)methyl]-3-hydroxypyrrolidine-1-carboxylate (6-3, 300 mg, 0.70 mmol, 1.00 equiv) and 4-nitrophenyl chloroformate (211.9 mg, 1.05 mmol, 1.5 equiv) in DCM (10 mL) was added Pyridine (166.6 mg, 2.13 mmol, 3 equiv) at room temperature. The resulting mixture was stirred at room temperature for overnight. The residue was purified by silica gel column chromatography, eluted with PE/EA (5:1) to afford tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (300 mg, 72.1%) as a white solid.

MS: m/z: Calc'd for C₂₈H₃₃ClN₂O₁₀ [M-100]⁺ 493. found 493.

### Synthetic Procedure of Intermediates Int-12 and Int-13

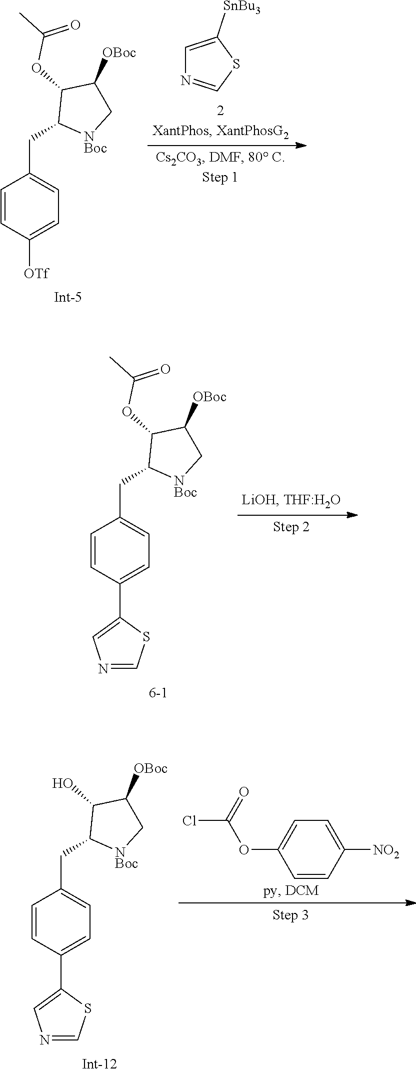

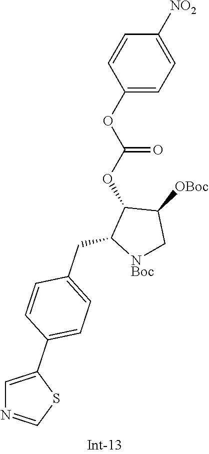

Step 1: To a stirred mixture of 5-(tributylstannyl)-1,3-thiazole (512.9 mg, 1.37 mmol, 2 equiv) and tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (Int-5, 400 mg, 0.685 mmol, 1.00 equiv) and CuCl (135.7 mg, 1.37 mmol, 2 equiv) in DMF (10 mL) was added XantPhos (158.6 mg, 0.27 mmol, 0.4 equiv) and XantPhos Pd G2 (121.8 mg, 0.13 mmol, 0.2 equiv) at room temperature under nitrogen atmosphere. The resulting mixture was stirred at 80° C. for overnight under nitrogen atmosphere. The residue was purified by reversed-phase flash chromatography to afford tert-butyl (2R,3S,4S)-3-(acetyloxy)-4-[(tert-butoxycarbonyl)oxy]-2-{[4-(1,3-thiazol-5-yl)phenyl]methyl}pyrrolidine-1-carboxylate (300 mg, 84.3% yield, 90% purity) as a white solid. MS: m/z: Calc'd for C26H34N207S [M+H]⁺519. found 519.

Step 2: To a stirred mixture of tert-butyl (2R,3S,4S)-3-(acetyloxy)-4-[(tert-butoxycarbonyl)oxy]-2-{[4-(1,3-thiazol-5-yl)phenyl]methyl}pyrrolidine-1-carboxylate (6-1, 300 mg, 0.57 mmol, 1 equiv) in THF (7.5 mL) and Water (2.5 mL) was added LiOH (41.5 mg, 1.73 mmol, 3 equiv) at room temperature. The resulting mixture was stirred at room temperature for 2h. The neutralized to pH 7 with conc. HCl. The reaction mixture was quenched with water and extracted with EA. The extracts were washed with brine, dried over anhydrous sodium sulfate, filtered and concentrated. This resulted in tert-butyl (2R,3S,4S)-4-[(tert-butoxycarbonyl)oxy]-3-hydroxy-2-{[4-(1,3-thiazol-5-yl)phenyl]methyl}pyrrolidine-1-carboxylate (250 mg, 90.6% yield, 90% purity) as a white oil. MS: m/z: Calc'd for C₂₄H₃₂N₂O₆S [M+H]⁺477. found 477.

Step 3: To a stirred solution of tert-butyl (2R,3S,4S)-4-[(tert-butoxycarbonyl)oxy]-3-hydroxy-2-{[4-(1,3-thiazol-5-yl)phenyl]methyl}pyrrolidine-1-carboxylate (210 mg, 0.44 mmol, 1 equiv) and 4-nitrophenyl carbonochloridate (133.2 mg, 0.66 mmol, 1.5 equiv) in DCM (15 mL) was added Pyridine (69.71 mg, 0.88 mmol, 2 equiv) at room temperature. The resulting mixture was stirred at room temperature for 3 h under nitrogen atmosphere. Desired product could be detected by LCMS. The residue was purified by Prep-TLC (PE:EA, 3:1). This resulted in tert-butyl (2R,3S,4S)-4-[(tert-butoxycarbonyl)oxy]-3-[(4-nitrophenoxycarbonyl)oxy]-2-{[4-(1,3-thiazol-5-yl)phenyl]methyl}pyrrolidine-1-carboxylate (140 mg, 49.5%) as a light yellow oil. MS: m/z: Calc'd for C31H35N3010S [M+H]⁺642. found 642.

### Example 4: Synthesis of Additional Compounds of the Present Disclosure

### General Procedure I: Substitution Reaction for Synthesis of Carbamate

To a stirred solution of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2, 0.12 mmol, 1 eq.) and corresponding amine (1 eq.) in ACN (5 mL) was added DIEA (3 eq.) at room temperature. The resulting mixture was stirred at room temperature for 12 h. After completion, the reaction mixture was concentrated in vacuo. The residue was purified by reversed column chromatography.

### General Procedure II: Boc Deprotection

The corresponding Boc-protected amine (1 equiv) was dissolved in anhydrous CH₂Cl₂ (5 mL/mmol), and TFA (5 mL/mmol) was added. The mixture was stirred at r.t. for 2 h. After removal of the volatiles, the oily residue was further dried under high vacuum. The residue was purified by prep-HPLC.

### General Procedure III: Cyclization Reaction for Synthesis of Substituted Triazoles

To a stirred solution of tert-butyl (2R,3S,4S)-4-[(tert-butoxycarbonyl)oxy]-2-[(4-methoxyphenyl)methyl]-3-(prop-2-yn-1-yloxy)pyrrolidine-1-carboxylate (Int-4, 80 mg, 0.17 mmol, 1 eq.) and corresponding azide (1 eq.) in MeOH (4 mL) was added CuSO₄·5H₂O (1 eq.) and sodium (5R)-5-[(1S)-1,2-dihydroxyethyl]-3,4-dihydroxy-2,5-dihydrofuran-2-one (2 eq.) at 0° C. The resulting mixture was stirred at room temperature for overnight. Desired product could be detected by LCMS. The resulting mixture was concentrated under reduced pressure. The residue was purified by reversed-phase flash chromatography.

General Procedure IV: Stille Coupling with Pd(PPh3)4, CuI and CsF

To a solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (Int-6, 0.51 mmol, 1 eq.) and corresponding bromide (1.5 eq.) in DMF (5 mL) were added CsF (2 eq.), Pd(PPh₃)₄ and CuI (0.1 eq.) at room temperature. The solution was heated at 80° C. under a nitrogen atmosphere for 16 h. Upon completion, the mixture was cooled to r.t., filtered and concentrated in vacuo. The residue was purified by reversed-phase flash chromatography.

General Procedure V: Hydrolysis Reaction with LiOH

To a solution of tert-butyl (2R,3S,4S)-3-(acetyloxy)-4-[(tert-butoxycarbonyl)oxy]-2-[(4-fluorophenyl)methyl]pyrrolidine-1-carboxylate (3-1, 0.22 mmol, 1 eq.) in THF (5 mL) and H₂O (0.5 mL) was added LiOH (3 eq.). The mixture was stirred at rt for 6 hours. Upon completion, the mixture was diluted with EA, washed with water. The organic layer was concentrated to give the crude product which was used in the next step without further purification.

### General Procedure VI: Addition Reaction: Isocyanates and Hydroxyl Groups

To a solution of tert-butyl (2R,3S,4S)-4-[(tert-butoxycarbonyl)oxy]-2-[(4-fluorophenyl)methyl]-3-hydroxypyrrolidine-1-carboxylate (Int-7, 0.11 mmol, 1 eq.), corresponding Isocyanates (1.5 eq.) in Toluene (3 mL) were added TEA (3 eq.) and DMAP (1 eq.). The reaction mixture was stirred at 80° C. for overnight. Desired product could be detected by LCMS. The resulting mixture was concentrated under vacuum. The residue was purified by reversed-phase flash chromatography.

### General Procedure VII: TBS Deprotection

The corresponding TBS-protected alcohol (1 equiv) was dissolved in anhydrous THF (5 mL/mmol), and Et₃N·3HF (5 mL/mmol) was added. The mixture was stirred at r.t. for 2 h. After removal of the volatiles, the residue was further purified by reversed phase flash with the following conditions (NH₄HCO₃/H₂O).

### Synthetic Procedure of (2R,3S,4S)-4-hydroxy-2-[(4-methoxyphenyl)methyl]pyrrolidin-3-yl N-(prop-2-yn-1-yl)carbamate (V0074)

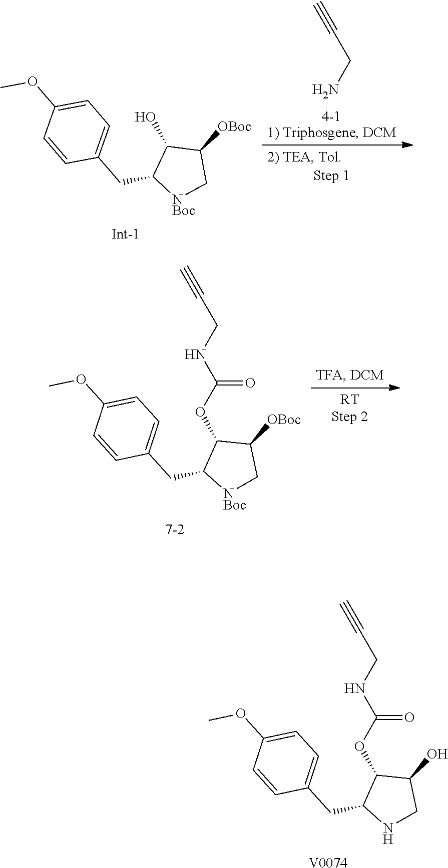

Step 1: To a stirred solution of 2-propynylamine (130 mg, 2.36 mmol, 5 eq.) in DCM (2 mL) was added Triphosgene (56.1 mg, 0.19 mmol, 0.4 eq.) and TEA (238.9 mg, 2.36 mmol, 5 eq.) at 0° C. and the resulting mixture was stirred at room temperature for 30 min. Then tert-butyl (2R,3S,4S)-4-[(tert-butoxycarbonyl)oxy]-3-hydroxy-2-[(4-methoxyphenyl)methyl]pyrrolidine-1-carboxylate (200 mg, 0.47 mmol, 1 eq.), DMAP (115.4 mg, 0.94 mmol, 2 eq.) and Toluene (5 mL) were added at room temperature. The resulting mixture was stirred at 80° C. for 12 h. Upon completion, the reaction mixture was quenched with water and extracted with EA. The organic phase was washed with brine, dried over anhydrous sodium sulfate, filtered and concentrated. The residue was purified by Prep-TLC to obtain tert-butyl (2R,3S,4S)-4-((tert-butoxycarbonyl)oxy)-2-(4-methoxybenzyl)-3-((prop-2-yn-1-ylcarbamoyl)oxy)pyrrolidine-1-carboxylate (80 mg, 51.42%) as a light-yellow oil. MS: m/z: Calc'd for C26H36N2O8 [M+H]⁺504. found 504.

Step 2: The compound was synthesized according to Boc Deprotection; General Procedure II using tert-butyl (2R,3S,4S)-4-((tert-butoxycarbonyl)oxy)-2-(4-methoxybenzyl)-3-((prop-2-yn-1-ylcarbamoyl)oxy)pyrrolidine-1-carboxylate (7-2, 80 mg, 0.16 mmol). The crude product was purified by Prep-HPLC to afford a yellow solid; yield: 26.1 mg (38.6%). MS: m/z: Calc'd for C₁₆H₂₀N₂O₄ [M+H]⁺305. found 305. ¹H NMR (400 MHz, DMSO-d₆) δ 9.50 (s, 1H), 9.07 (s, 1H), 7.71-7.69 (m, 1H), 7.22 (d, J=8.4 Hz, 2H), 6.90 (d, J=8.0 Hz, 2H), 5.99 (s, 1H), 4.76-4.68 (m, 1H), 4.21-4.19 (m, 1H), 3.98 (s, 1H), 3.92-3.80 (m, 2H), 3.74 (s, 4H), 3.57-3.50 (m, 1H), 3.21 (s, 1H), 3.07-3.05 (m, 1H), 3.01-2.83 (m, 2H).

Prep-HPLC purification conditions: Column: UV 254 nm/220 nm Xselect CSH Prep OBD C18 Column, 30*150 mm, 5 μm; Mobile Phase A: Water (0.1% TFA), Mobile Phase B: ACN 60 mL/min; Gradient: 2% B to 26% B in 10 min 6.43 Wave Length: 254 nm/220 nm; RT1(min): 6.2

### Synthetic Procedure of (2R,3S,4S)-4-hydroxy-2-[(4-methoxyphenyl)methyl]pyrrolidin-3-yl N-(2-{2-hydroxy-6-azaspiro[3.4]octan-6-yl}ethyl)carbamate (V187642)

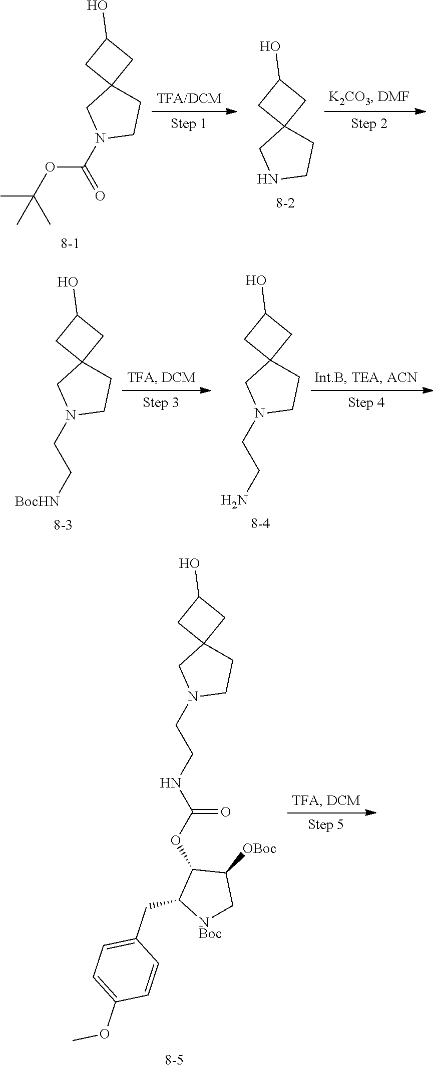

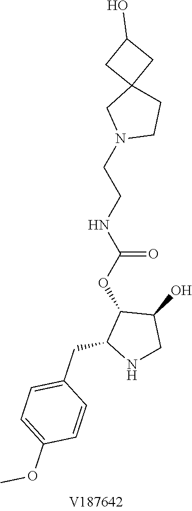 [SMILES: COc1ccc(C[C@H]2NC[C@H](O)[C@H]2OC(=O)NCCN2CCC3(CC(O)C3)C2)cc1]

Step 1: To a solution of tert-butyl 2-hydroxy-6-azaspiro[3.4]octane-6-carboxylate (8-1, 300 mg, 1.32 mmol, 1 eq.) in DCM (5 mL) was added TFA (1 mL). The mixture was stirred at room temperature for 1 hour. LCMS showed the reaction was complete. The mixture was concentrated to give the crude product which was used directly in the next step without further purification. MS: m/z: Calc'd for C7H13NO [M+H]⁺128. found 128.

Step 2: To a solution of 6-azaspiro[3.4]octan-2-ol (8-2, 160 mg, 1.26 mmol, 1 eq.) and tert-butyl N-(2-bromoethyl)carbamate (423 mg, 1.89 mmol, 1.5 eq.) in DMF (5 mL) was added K₂CO₃ (869 mg, 6.29 mmol, 5 eq.). The mixture was stirred at room temperature for overnight. The mixture was diluted with EA, washed with water, dried, filtered and concentrated to give the crude product which was used directly in the next step without further purification as a yellow oil. MS: m/z: Calc'd for C₁₄H₂₆N₂O₃ [M+H]⁺271. found 271.

Step 3: To a solution of tert-butyl N-(2-{2-hydroxy-6-azaspiro[3.4]octan-6-yl}ethyl)carbamate (8-3, 55 mg, 0.20 mmol, 1 eq.) in DCM (5 mL) was added TFA (1 mL). The mixture was stirred at room temperature for 1 hour. The mixture was concentrated to give the crude product which was used directly in the next step without further purification. MS: m/z: Calc'd for C9H18N₂O [M+H]⁺171. found 171.

### Steps 4 and 5

The title compound was prepared in 26.1% overall yield as a light yellow semi-solid according to Substitution reaction for synthesis of Carbamate; General Procedure I using 6-(2-aminoethyl)-6-azaspiro[3.4]octan-2-ol (8-4) in STEP 4; Boc Deprotection; General Procedure II in STEP 5. MS: m/z: Calc'd for C₂₂H₃₃N₃O₅ [M+H]⁺420. found 420. ¹H NMR (400 MHz, Methanol-d₄) δ 7.25 (d, J=8.3 Hz, 2H), 6.94 (d, J=8.1 Hz, 2H), 5.01 (d, J=3.4 Hz, 1H), 4.42 (d, J=4.2 Hz, 1H), 4.36-4.13 (m, 2H), 3.80 (s, 3H), 3.76-3.71 (m, 2H), 3.64-3.49 (m, 3H), 3.39-3.32 (m, 3H), 3.27-3.16 (m, 2H), 3.17-3.07 (m, 1H), 3.04-2.94 (m, 1H), 2.57-2.35 (m, 2H), 2.29-1.94 (m, 4H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 17% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-(1H-1,3-benzodiazol-2-yl)carbamate (V187754)

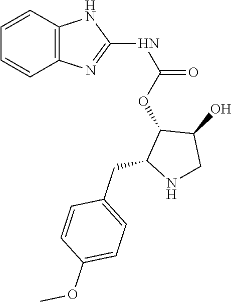 [SMILES: COc1ccc(C[C@H]2NC[C@H](O)[C@H]2OC(=O)Nc2nc3ccccc3[nH]2)cc1]

The title compound was prepared in 27.0% overall yield as a white solid according to Substitution reaction for synthesis of Carbamate; General Procedure I using 2-aminobenzimidazole in STEP 1; Boc Deprotection; General Procedure II in STEP 2. MS: m/z Calc'd for C₂₀H₂₂N₄O₄ [M+H]⁺383. Found, 383. ¹H NMR (400 MHz, Methanol-d₄) δ 9.62-9.60 (m, 2H), 7.50 (d, J=5.9 Hz, 2H), 7.35-7.17 (m, 4H), 6.95-6.85 (m, 2H), 6.05 (s, 1H), 4.96 (d, J=3.9 Hz, 1H), 4.39 (d, J=3.9 Hz, 1H), 4.07 (s, 1H), 3.71 (s, 3H), 3.47 (d, J=5.0 Hz, 2H), 3.11-2.97 (m, 3H).

Prep-HPLC purification conditions: Column: Xselect CSH Prep C18 Column, 30*150 mm, 5 μm; Mobile Phase A: Water (0.05% HCl), Mobile Phase B: ACN; Flow rate: 60 mL/min; Gradient: 2% B to 25% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-(1,3-oxazol-4-ylmethyl)carbamate (V187767)

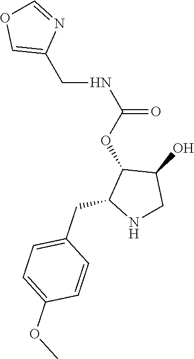 [SMILES: COc1ccc(C[C@H]2NC[C@H](O)[C@H]2OC(=O)NCc2cocn2)cc1]

The title compound was prepared in 25.8% overall yield as a white solid according to Substitution reaction for synthesis of Carbamate; General Procedure I using 1-(1,3-oxazol-4-yl)methanamine in STEP 1; Boc Deprotection; General Procedure II in STEP 2. MS: m/z Calc'd for C₁₇H₂₁N₃O₅ [M+H]⁺348. Found, 348. ¹H NMR (400 MHz, Methanol-d₄) δ 8.21 (s, 1H), 7.86 (s, 1H), 7.21 (d, J=8.3 Hz, 2H), 6.90 (d, J=8.3 Hz, 2H), 4.95 (d, J=3.5 Hz, 1H), 4.40 (d, J=4.2 Hz, 1H), 4.38-4.23 (m, 2H), 4.18-4.14 (m, 1H), 3.79 (s, 3H), 3.58 (dd, J=12.6, 4.3 Hz, 1H), 3.21 (d, J=12.6 Hz, 1H), 3.10 (dd, J=14.1, 7.3 Hz, 1H), 2.96 (dd, J=14.1, 8.4 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.68.

### Synthetic Procedure of (2R,3S,4S)-4-hydroxy-2-(4-methoxybenzyl)pyrrolidin-3-yl azetidin-3-ylcarbamate (V187679)

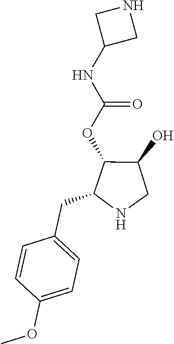 [SMILES: COc1ccc(C[C@H]2NC[C@H](O)[C@H]2OC(=O)NC2CNC2)cc1]

The title compound was prepared in 38.6% overall yield as yellow solid according to Substitution reaction for synthesis of Carbamate; General Procedure I using tert-butyl 3-aminoazetidine-1-carboxylate in STEP 1; Boc Deprotection; General Procedure II using ZnBr₂ instead of TFA in STEP 2. MS: m/z Calc'd for C₁₆H₂₃N₃O₄ [M+H]⁺322. found 322. ¹H NMR (400 MHz, Methanol-d₄) δ 8.58 (s, 2H), 7.23 (d, J=8.5 Hz, 2H), 6.96-6.89 (m, 2H), 4.94 (s, 1H), 4.64-4.54 (m, 1H), 4.39-4.16 (m, 5H), 4.10 (s, 1H), 3.79 (s, 3H), 3.56 (dd, J=12.6, 4.5 Hz, 1H), 3.16 (d, J=12.5 Hz, 1H), 3.12-2.93 (m, 2H).

Prep-HPLC purification conditions: Column: Welch Ultimate XB-C18 50*250, 10 μm; Mobile Phase A: Water (0.1% FA), Mobile Phase B: ACN; Flow rate: 60 mL/min; Gradient: 0% B to 10% B in 20 min; Wave Length: 254 nm/220 nm; RT1(min): 15.35.

### Synthetic Procedure of (2R,3S,4S)-4-hydroxy-2-(4-methoxybenzyl)pyrrolidin-3-yl (((S)-pyrrolidin-3-yl)methyl)carbamate (V187686)

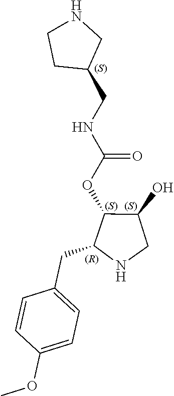

The title compound was prepared in 55.2% overall yield as yellow solid according to Substitution reaction for synthesis of Carbamate; General Procedure I using tert-butyl (R)-3-(aminomethyl)pyrrolidine-1-carboxylate in STEP 1; Boc Deprotection; General Procedure II using HCl (4 M in dioxane) instead of TFA in STEP 2. MS: m/z Calc'd for C₁₈H₂₇N₃O₄ [M+H]⁺350. Found, 350. ¹H NMR (400 MHz, Methanol-d₄) δ 8.56 (s, 2H), 7.27-7.19 (m, 2H), 6.96-6.88 (m, 2H), 4.35 (d, J=4.4 Hz, 1H), 4.89 (s, 1H), 4.14-4.04 (m, 1H), 3.80 (s, 3H), 3.57 (dd, J=12.6, 4.5 Hz, 1H), 3.48-3.35 (m, 2H), 3.33-3.26 (m, 3H), 3.26-3.17 (m, 1H), 3.17-2.93 (m, 3H), 2.70-2.55 (m, 1H), 2.25-2.12 (m, 1H), 1.86-1.72 (m, 1H).

Prep-HPLC purification conditions: Column: Welch Ultimate XB-C18 50*250, 10 μm; Mobile Phase A: Water (0.1% FA), Mobile Phase B: ACN; Flow rate: 60 mL/min; Gradient: 0% B to 10% B in 20 min; Wave Length: 254 nm/220 nm; RT1(min): 14.12.

### Synthetic Procedure of (2R,3S,4S)-4-hydroxy-2-(4-methoxybenzyl)pyrrolidin-3-yl ((R)-pyrrolidin-3-yl)carbamate (V187688)

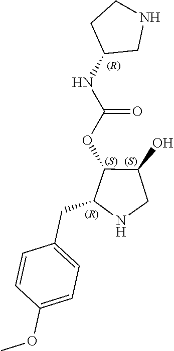

The title compound was prepared in 45.7% overall yield as yellow solid according to Substitution reaction for synthesis of Carbamate; General Procedure I using tert-butyl (R)-3-aminopyrrolidine-1-carboxylate in STEP 1; Boc Deprotection; General Procedure II using HCl (4 M in dioxane) instead of TFA in STEP 2. MS: m/z Calc'd for C₁₇H₂₅N₃O₄ [M+H]⁺336. Found, 336. ¹H NMR (400 MHz, Methanol-d₄) δ 8.54 (s, 1H), 7.26-7.20 (m, 2H), 6.97-6.88 (m, 2H), 4.94 (d, J=3.4 Hz, 2H), 4.39-4.31 (m, 2H), 4.17-4.07 (m, 1H), 3.80 (s, 3H), 3.60-3.44 (m, 3H), 3.44-3.34 (m, 1H), 3.18 (d, J=12.6 Hz, 1H), 3.09 (dd, J=14.1, 7.2 Hz, 1H), 2.98 (dd, J=14.1, 8.4 Hz, 1H), 2.41-2.28 (m, 1H), 2.09-2.07 (m, 1H).

Prep-HPLC purification conditions: Column: Welch Ultimate XB-C18 50*250, 10 μm; Mobile Phase A: Water (0.1% FA), Mobile Phase B: ACN; Flow rate: 60 mL/min; Gradient: 0% B to 10% B in 20 min; Wave Length: 254 nm/220 nm; RT1(min): 14.

### Synthetic Procedure of (2R,3S,4S)-4-hydroxy-2-[(4-methoxyphenyl)methyl]pyrrolidin-3-yl N-(azetidin-3-ylmethyl)carbamate (V187599)

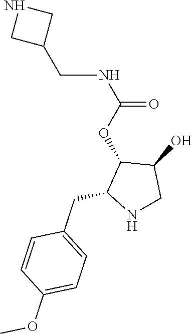 [SMILES: COc1ccc(C[C@H]2NC[C@H](O)[C@H]2OC(=O)NCC2CNC2)cc1]

The title compound was prepared in 38.0% overall yield as a white solid according to Substitution reaction for synthesis of Carbamate; General Procedure I using tert-butyl 3-(aminomethyl)azetidine-1-carboxylate in STEP 1; Boc Deprotection; General Procedure II in STEP 2. MS: m/z Calc'd for C₁₇H₂₅N₃O₄ [M+H]⁺336. Found, 336. ¹H NMR (400 MHz, Methanol-d₄) δ 7.24 (d, J=8.3 Hz, 2H), 6.93 (d, J=8.3 Hz, 2H), 4.96 (d, J=3.4 Hz, 1H), 4.39 (d, J=4.2 Hz, 1H), 4.19-4.11 (m, 3H), 3.97-3.93 (m, 2H), 3.80 (s, 3H), 3.59 (dd, J=12.7, 4.4 Hz, 1H), 3.43 (d, J=6.6 Hz, 2H), 3.22 (d, J=12.6 Hz, 1H), 3.16-3.08 (m, 2H), 2.99 (dd, J=14.1, 8.6 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 32% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-(1,3-thiazol-2-yl)carbamate Hydrochloride (V187752)

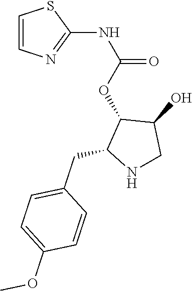 [SMILES: COc1ccc(C[C@H]2NC[C@H](O)[C@H]2OC(=O)Nc2nccs2)cc1]

The title compound was prepared in 46.4% overall yield as a white solid according to Substitution reaction for synthesis of Carbamate; General Procedure I using aminothiazole in STEP 1; Boc Deprotection; General Procedure II in STEP 2. MS: m/z Calc'd for C₁₆H₁₉N₃O₄S [M+H]350. Found, 350. ¹H NMR (400 MHz, DMSO-d₆) δ 10.01 (s, 1H), 9.68 (s, 1H), 7.44 (d, J=3.5 Hz, 1H), 7.30-7.21 (m, 3H), 6.90-6.86 (m, 2H), 4.82 (d, J=3.9 Hz, 1H), 4.36 (d, J=3.9 Hz, 1H), 4.06 (d, J=10.3 Hz, 1H), 3.71-3.69 (m, 3H), 3.58 (s, 1H), 3.48-3.34 (m, 1H), 3.15-2.96 (m, 3H).

Prep-HPLC purification conditions: Column: Xselect CSH Prep C18 Column, 30*150 mm, 5 μm; Mobile Phase A: Water (0.05% HCl), Mobile Phase B: ACN; Flow rate: 60 mL/min; Gradient: 2% B to 26% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-[2-(pyridin-4-yl)ethyl]carbamate (V187807)

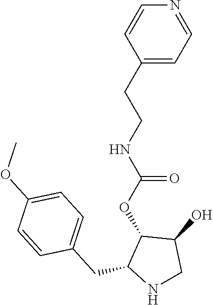 [SMILES: COc1ccc(C[C@H]2NC[C@H](O)[C@H]2OC(=O)NCCc2ccncc2)cc1]

The title compound was prepared in 23.4% overall yield as a light yellow oil according to Substitution reaction for synthesis of Carbamate; General Procedure I using 4-pyridine etheneamine in STEP 1; Boc Deprotection; General Procedure II in STEP 2. MS: m/z Calc'd for C₂₀H₂₅N₃O₄ [M+H]⁺372. found 372. ¹H NMR (400 MHz, Methanol-d₄) δ 8.72 (s, 2H), 7.92 (d, J=5.7 Hz, 2H), 7.20 (d, J=8.2 Hz, 2H), 6.92 (d, J=8.5 Hz, 2H), 4.92 (d, J=3.4 Hz, 2H), 4.34 (d, J=4.2 Hz, 1H), 4.13 (s, 1H), 3.80 (s, 3H), 3.62-3.51 (m, 3H), 3.22-3.11 (m, 3H), 3.05 (dd, J=14.1, 7.0 Hz, 1H), 2.91 (dd, J=14.2, 8.6 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 8.68.

### Synthetic Procedure of N′-({[(2R,3S,4S)-4-hydroxy-2-[(4-methoxyphenyl)methyl]pyrrolidin-3-yl]oxy}carbonyl)benzohydrazide (V187812)

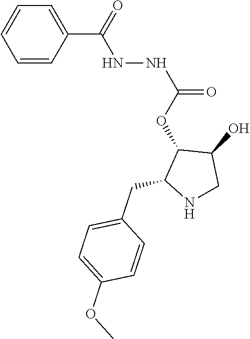 [SMILES: COc1ccc(C[C@H]2NC[C@H](O)[C@H]2OC(=O)NNC(=O)c2ccccc2)cc1]

The title compound was prepared in 42.9% overall yield as a white solid according to Substitution reaction for synthesis of Carbamate; General Procedure I using benzohydrazide in STEP 1; Boc Deprotection; General Procedure II in STEP 2. MS: m/z Calc'd for C₂₀H₂₃N₃O₅ [M+H]⁺386. found 386. ¹H NMR (400 MHz, Methanol-d₄) δ 7.90 (dd, J=7.2, 1.8 Hz, 2H), 7.61 (t, J=7.4 Hz, 1H), 7.51 (t, J=7.6 Hz, 2H), 7.22 (d, J=8.1 Hz, 2H), 6.87 (d, J=8.1 Hz, 2H), 4.71 (d, J=3.5 Hz, 1H), 4.26 (s, 1H), 3.78 (s, 3H), 3.54 (s, 1H), 3.44-3.36 (m, 1H), 2.93-2.67 (m, 3H).

Prep-HPLC purification conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 6% B to 36% B in 10 min; Wave Length: 254 nm/220 nm; RT1(min): 8.7.

### Synthetic Procedure of (2R,3S,4S)-4-hydroxy-2-[(4-methoxyphenyl)methyl]pyrrolidin-3-yl N-[2-(5-chloro-3H-1,3-benzodiazol-2-yl)ethyl]carbamate (V187821)

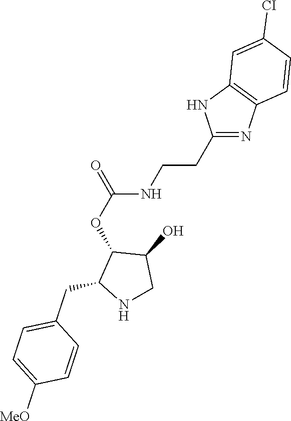

The title compound was prepared in 22.1% overall yield as a white solid according to Substitution reaction for synthesis of Carbamate; General Procedure I using 2-(5-chloro-3H-1,3-benzodiazol-2-yl)ethanamine in STEP 1; Boc Deprotection; General Procedure II in STEP 2. MS: m/z Calc'd for C₂₂H₂₅ClN₄O₄[M+H]⁺445. Found, 445. ¹H NMR (400 MHz, DMSO-d₆) δ 7.78 (d, J=1.9 Hz, 1H), 7.70 (d, J=8.6 Hz, 1H), 7.43 (dd, J=8.6, 2.0 Hz, 1H), 7.15-7.13 (m, 2H), 6.88-6.84 (m, 2H), 4.65 (d, J=3.7 Hz, 1H), 4.15 (d, J=4.1 Hz, 1H), 3.93-3.91 (m, 1H), 3.58-3.51 (m, 2H), 3.34 (dd, J=12.7, 4.3 Hz, 1H), 3.25-3.14 (m, 2H), 3.06 (d, J=12.5 Hz, 1H), 2.84-2.82 (m, 2H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 29% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-{[1,1′-biphenyl]-3-ylmethyl}carbamate (V187982)

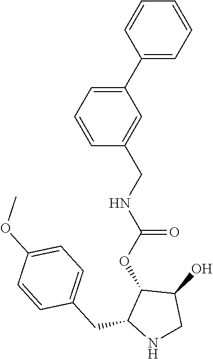 [SMILES: COc1ccc(C[C@H]2NC[C@H](O)[C@H]2OC(=O)NCc2cccc(-c3ccccc3)c2)cc1]

The title compound was prepared in 22.1% overall yield as a white solid according to Substitution reaction for synthesis of Carbamate; General Procedure I using 1-{[1,1′-biphenyl]-3-yl}methanamine in STEP 1; Boc Deprotection; General Procedure II in STEP 2. MS: m/z Calc'd for C₂₆H₂₈N₂O₄ [M+H]⁺432. found 432. ¹H NMR (400 MHz, Methanol-d₄) δ 7.70-7.60 (m, 3H), 7.57 (d, J=7.8 Hz, 1H), 7.50-7.39 (m, 3H), 7.35 (dd, J=7.8, 6.0 Hz, 2H), 7.17 (d, J=8.5 Hz, 2H), 6.82 (d, J=8.5 Hz, 2H), 4.96 (d, J=3.5 Hz, 1H), 4.49-4.47 (m, 1H), 4.41 (d, J=4.2 Hz, 1H), 4.36-4.34 (m, 1H), 4.20-4.11 (m, 1H), 3.74 (s, 3H), 3.58 (dd, J=12.6, 4.4 Hz, 1H), 3.23 (d, J=12.6 Hz, 1H), 3.11 (dd, J=14.0, 7.6 Hz, 1H), 2.96 (dd, J=14.0, 8.2 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 18% B to 48% B in 10 min; Wave Length: 254 nm/220 nm; RT1(min): 8.35.

### Synthetic Procedure of (2R,3S,4S)-4-hydroxy-2-[(4-methoxyphenyl)methyl]pyrrolidin-3-yl N-[2-(azetidin-3-yl)ethyl]carbamate (V187600)

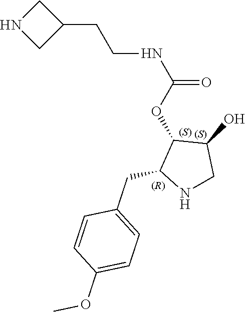

The title compound was prepared in 51.4% overall yield as an off-white semi-solid according to Substitution reaction for synthesis of Carbamate; General Procedure I using tert-butyl 3-(2-aminoethyl)azetidine-1-carboxylate in STEP 1; Boc Deprotection; General Procedure II in STEP 2. MS: m/z Calc'd for C₁₈H₂₇NO₄[M+H]⁺350, Found 350. ¹H NMR (400 MHz, Methanol-d₄) δ 7.27-7.20 (m, 2H), 6.97-6.89 (m, 2H), 4.95 (s, 1H), 4.38 (d, 1H), 4.16-4.14 (m, 3H), 3.85-3.80 (m, 3H), 3.58-3.56 (m, 1H), 3.28-3.06 (m, 4H), 3.09-2.93 (m, 2H), 1.91 (s, 2H).

Prep-HPLC purification conditions: Column: Welch Ultimate AQ-C18 50*250, 10 μm; Mobile Phase A: Water (0.05% TFA), Mobile Phase B: ACN; Flow rate: 60 mL/min; Gradient: 2% B to 35% B in 20 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-(1H-imidazol-2-yl)carbamate (V187755)

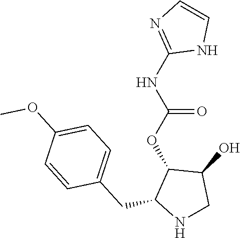 [SMILES: COc1ccc(C[C@H]2NC[C@H](O)[C@H]2OC(=O)Nc2ncc[nH]2)cc1]

The title compound was prepared in 9.6% overall yield as a white solid according to Substitution reaction for synthesis of Carbamate; General Procedure I using 1H-imidazol-2-amine in STEP 1; Boc Deprotection; General Procedure II in STEP 2. MS: m/z Calc'd for C₁₆H₂₀N₄O₄ [M+H]⁺333. found 333. ¹H NMR (400 MHz, Methanol-d₄) δ 7.30-7.05 (m, 2H), 6.98-6.72 (m, 2H), 6.52 (s, 2H), 4.62 (d, J=3.7 Hz, 1H), 4.35-4.09 (m, 1H), 3.77 (d, J=5.9 Hz, 3H), 3.69-3.36 (m, 2H), 3.24-2.54 (m, 3H), 1.77-1.20 (m, 1H).

Prep-HPLC purification conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 3% B to 30% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-(1H-imidazol-2-ylmethyl)carbamate (V187815)

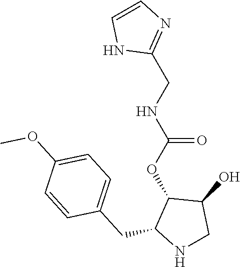 [SMILES: COc1ccc(C[C@H]2NC[C@H](O)[C@H]2OC(=O)NCc2ncc[nH]2)cc1]

The title compound was prepared in 34.5% overall yield as a yellow solid according to Substitution reaction for synthesis of Carbamate; General Procedure I using 1-(1H-imidazol-2-yl)methanamine in STEP 1; Boc Deprotection; General Procedure II in STEP 2. MS: m/z Calc'd for C₁₇H₂₂N₄O₄ [M+H]⁺347. found 347. ¹H NMR (400 MHz, Methanol-d₄) δ 7.51 (s, 2H), 7.26-7.19 (m, 2H), 6.90 (d, J=8.3 Hz, 2H), 4.99 (d, J=3.3 Hz, 1H), 4.66 (d, J=2.0 Hz, 2H), 4.41 (d, J=4.2 Hz, 1H), 4.17-4.05 (m, 1H), 3.79 (s, 3H), 3.61-3.48 (m, 1H), 3.22 (d, J=12.6 Hz, 1H), 3.11 (dd, J=14.2, 6.9 Hz, 1H), 3.00-2.89 (m, 1H).

Prep-HPLC purification conditions: Column: XBridge Prep OBD C18 Column, 19*250 mm, 5 μm; Mobile Phase A: Water (0.05% TFA), Mobile Phase B: ACN; Flow rate: 25 mL/min; Gradient: 3% B to 26% B in 8 min; Wave Length: 254 nm/220 nm; RT1(min): 6.88.

### Synthetic Procedure (2R,3S,4S)-4-hydroxy-2-[(4-methoxyphenyl)methyl]pyrrolidin-3-yl N-[2-(1H-imidazol-2-yl)ethyl]carbamate (V187816

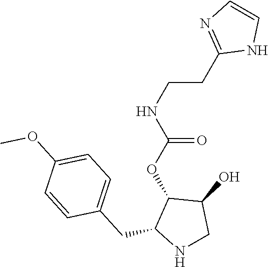 [SMILES: COc1ccc(C[C@H]2NC[C@H](O)[C@H]2OC(=O)NCCc2ncc[nH]2)cc1]

The title compound was prepared in 33.3% overall yield as a white solid according to Substitution reaction for synthesis of Carbamate; General Procedure I using 2-(1H-imidazol-2-yl)ethanamine in STEP 1; Boc Deprotection; General Procedure II in STEP 2. MS: m/z Calc'd for C₁₈H₂₄N₄O₄ [M+H]⁺361. found 361. ¹H NMR (400 MHz, Methanol-d₄) δ 7.46 (d, J=1.9 Hz, 2H), 7.29-7.17 (m, 2H), 6.92 (d, J=8.4 Hz, 2H), 4.90 (s, 1H), 4.32 (d, J=4.1 Hz, 1H), 4.17-4.09 (m, 1H), 3.79 (s, 3H), 3.63-3.51 (m, 3H), 3.26-3.16 (m, 3H), 3.05 (dd, J=14.2, 6.8 Hz, 1H), 2.92-2.79 (m, 1H).

Prep-HPLC purification conditions: Column: XBridge Prep OBD C18 Column, 19*250 mm, 5 μm; Mobile Phase A: Water (0.05% TFA), Mobile Phase B: ACN; Flow rate: 25 mL/min; Gradient: 3% B to 26% B in 8 min; Wave Length: 254 nm/220 nm; RT1(min): 7.23.

### Synthetic Procedure of (2R,3S,4S)-4-hydroxy-2-[(4-methoxyphenyl)methyl]pyrrolidin-3-yl N-[2-(4-methyl-3H-imidazol-2-yl)ethyl]carbamate (V187818)

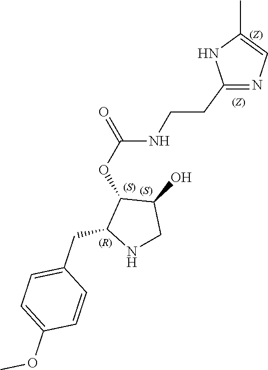

The title compound was prepared in 13.5% overall yield as a white solid according to Substitution reaction for synthesis of Carbamate; General Procedure I using 2-(4-methyl-3H-imidazol-2-yl)ethanamine in STEP 1; Boc Deprotection; General Procedure II in STEP 2. MS: m/z Calc'd for C₁₉H₂₆N₄O₄[M+H]⁺375, Found 375. ¹H NMR (400 MHz, Methanol-d₄) δ 7.28-7.18 (m, 2H), 7.14 (d, 1H), 6.97-6.88 (m, 2H), 4.97-4.91 (m, 1H), 4.36-4.31 (m, 1H), 4.14 (s, 1H), 3.80 (s, 3H), 3.57-3.55 (m, 3H), 3.24-3.12 (m, 3H), 3.06-3.04 (m, 1H), 2.91-2.89 (m, 1H), 2.31 (s, 3H).

Prep-HPLC purification conditions: Column: XBridge Prep OBD C18 Column, 19*250 mm, 5 μm; Mobile Phase A: Water (0.05% TFA), Mobile Phase B: ACN; Flow rate: 25 mL/min; Gradient: 3% B to 26% B in 8 min; Wave Length: 254 nm/220 nm; RT1(min): 7.23.

### Synthetic Procedure of (2R,3S,4S)-4-hydroxy-2-[(4-methoxyphenyl)methyl]pyrrolidin-3-yl N-[2-(imidazol-1-yl)ethyl]carbamate (V187819)

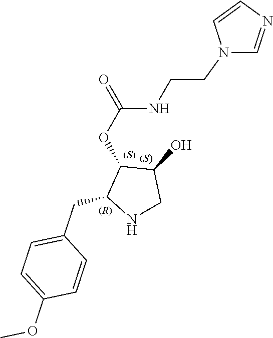

The title compound was prepared in 55.3% overall yield as a yellow oil according to Substitution reaction for synthesis of Carbamate; General Procedure I using O-aminoethylimidazole in STEP 1; Boc Deprotection; General Procedure II in STEP 2. MS: m/z Calc'd for C₁₈H₂₄N₄O₄[M+H]⁺361, Found 361. ¹H NMR (400 MHz, Methanol-d₄) δ 8.93 (s, 1H), 7.65 (s, 1H), 7.55 (s, 1H), 7.25-7.18 (m, 2H), 6.98-6.89 (m, 2H), 4.40 (d, 2H), 4.35 (d, 2H), 4.15-4.13 (m, 1H), 3.80 (s, 3H), 3.65 (s, 2H), 3.56-3.54 (m, 1H), 3.31 (s, 1H), 3.21 (d, 1H), 3.07-3.05 (m, 1H), 2.92-2.90 (m, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 5% B to 95% B in 20 min; Wave Length: 254 nm/220 nm; RT1(min): 11.26.

### Synthetic Procedure of (2R,3S,4S)-4-hydroxy-2-[(4-methoxyphenyl)methyl]pyrrolidin-3-yl N-[2-(1H-1,3-benzodiazol-2-yl)ethyl]carbamate (V187820)

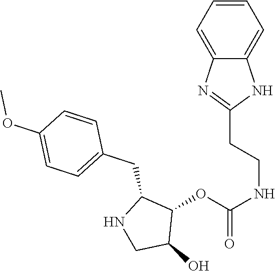 [SMILES: COc1ccc(C[C@H]2NC[C@H](O)[C@H]2OC(=O)NCCc2nc3ccccc3[nH]2)cc1]

The title compound was prepared in 32.4% overall yield as a white solid according to Substitution reaction for synthesis of Carbamate; General Procedure I using 2-(1H-1,3-benzodiazol-2-yl)ethanamine in STEP 1; Boc Deprotection; General Procedure II in STEP 2. MS: m/z Calc'd for C₂₂H₂₆N₄O₄ [M+H]⁺411. found 411. ¹H NMR (400 MHz, Methanol-d₄) δ 7.77-7.68 (m, 2H), 7.53-7.41 (m, 2H), 7.15 (d, J=8.2 Hz, 2H), 6.92-6.84 (m, 2H), 4.29-4.22 (m, 1H), 4.07-3.98 (m, 1H), 3.78 (s, 3H), 3.74-3.61 (m, 2H), 3.53-3.43 (m, 1H), 3.41-3.31 (m, 2H), 3.20-3.12 (m, 1H), 2.99-2.89 (m, 1H), 2.84-2.71 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 24% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-[(3,3-difluorocyclobutyl)methyl]carbamate (V187862)

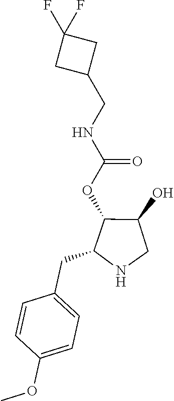 [SMILES: COc1ccc(C[C@H]2NC[C@H](O)[C@H]2OC(=O)NCC2CC(F)(F)C2)cc1]

The title compound was prepared in 32.4% overall yield as a white solid according to Substitution reaction for synthesis of Carbamate; General Procedure I using 1-(3,3-difluorocyclobutyl)methanamine hydrochloride in STEP 1; Boc Deprotection; General Procedure II in STEP 2. MS: m/z Calc'd for C₁₈H₂₄F₂NO₄[M+H]⁺371, Found 371. ¹H NMR (400 MHz, Methanol-d₄) δ 7.23 (dd, 2H), 6.97-6.89 (m, 2H), 4.93 (s, 1H), 4.42-4.33 (m, 1H), 4.21-4.13 (m, 1H), 3.78 (s, 3H), 3.56 (s, 1H), 3.32-3.18 (m, 3H), 3.09 (s, 1H), 2.97-2.95 (m, 1H), 2.71-2.58 (m, 2H), 2.41-2.30 (m, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 8% B to 38% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-[(3-fluoroazetidine-3-yl)methyl]carbamate (V187877)

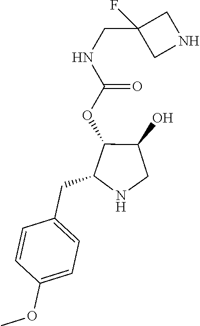 [SMILES: COc1ccc(C[C@H]2NC[C@H](O)[C@H]2OC(=O)NCC2(F)CNC2)cc1]

The title compound was prepared in 31.9% overall yield as a white solid according to Substitution reaction for synthesis of Carbamate; General Procedure I using tert-butyl 3-(aminomethyl)-3-fluoroazetidine-1-carboxylate in STEP 1; Boc Deprotection; General Procedure II in STEP 2. MS: m/z Calc'd for C₁₇H₂₄FNO₄[M+H]⁺354, Found 354. ¹H NMR (400 MHz, Methanol-d₄) δ 7.14 (d, 2H), 6.84-6.82 (m, 2H), 4.63-4.61 (m, 1H), 4.15-4.11 (m, 1H), 3.78 (s, 4H), 3.81-3.48 (m, 6H), 3.38-3.31 (m, 1H), 2.88-2.66 (m, 3H).

Prep-HPLC purification conditions: Column: XBridge Prep OBD C18 Column, 50*250 mm, 10 μm; Mobile Phase A: Water (10 mmol/LNH₄HCO₃), Mobile Phase B: ACN; Flow rate: 60 mL/min; Gradient: 2% B to 35% B in 20 min; Wave Length: 254 nm/220 nm; RT1(min): 18.08.

### Synthetic Procedure of (2R,3S,4S)-4-hydroxy-2-[(4-methoxyphenyl)methyl]pyrrolidin-3-yl N-(pyridazin-3-ylmethyl)carbamate (V187916)

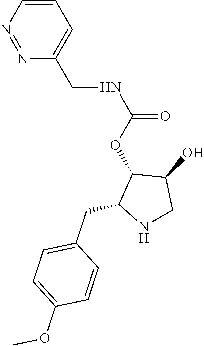 [SMILES: COc1ccc(C[C@H]2NC[C@H](O)[C@H]2OC(=O)NCc2cccnn2)cc1]

The title compound was prepared in 16.4% overall yield as a yellow oil according to Substitution reaction for synthesis of Carbamate; General Procedure I using 1-(pyridazin-3-yl)methanamine in STEP 1; Boc Deprotection; General Procedure II in STEP 2. MS: m/z Calc'd for C₁₈H₂₂N₄O₄[M+H]⁺359, Found 359. ¹H NMR (400 MHz, Methanol-d₄) δ 9.19-9.13 (m, 1H), 7.77-7.75 (m, 2H), 7.24-7.22 (m, 2H), 6.91-6.89 (m, 2H), 4.97-4.95 (m, 1H), 4.67 (s, 2H), 4.43-4.41 (m, 1H), 4.18-4.16 (m, 1H), 3.80 (s, 3H), 3.62-6.60 (m, 1H), 3.24-3.22 (m, 1H), 3.14-3.12 (m, 1H), 3.00-2.98 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 27% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-(pyridazin-4-ylmethyl)carbamate (V187917)

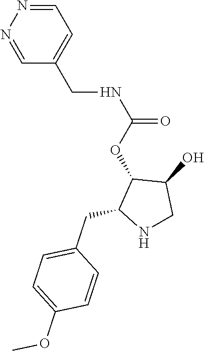 [SMILES: COc1ccc(C[C@H]2NC[C@H](O)[C@H]2OC(=O)NCc2ccnnc2)cc1]

The title compound was prepared in 37.8% overall yield as a brown oil according to Substitution reaction for synthesis of Carbamate; General Procedure I using 1-(pyridazin-4-yl)methanamine in STEP 1; Boc Deprotection; General Procedure II in STEP 2. MS: m/z Calc'd for C₁₈H₂₂N₄O₄[M+H]⁺359, Found 359. ¹H NMR (400 MHz, Methanol-d₄) δ 9.26-9.18 (m, 2H), 7.81-7.72 (m, 1H), 7.22-7.20 (m, 2H), 6.90-6.88 (m, 2H), 4.96-4.98 (m, 1H), 4.47-4.42 (m, 3H), 4.20-4.18 (m, 1H), 3.80 (s, 3H), 3.61-3.59 (m, 1H), 3.25-3.23 (m, 1H), 3.13-3.11 (m, 1H), 3.01-2.99 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 18% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-[(4-iodophenyl)methyl]carbamate (V187928)

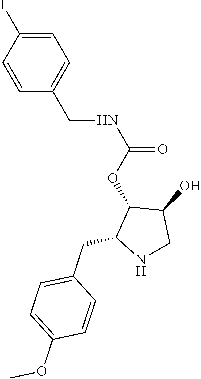 [SMILES: COc1ccc(C[C@H]2NC[C@H](O)[C@H]2OC(=O)NCc2ccc(I)cc2)cc1]

The title compound was prepared in 25.9% overall yield as a white solid according to Substitution reaction for synthesis of Carbamate; General Procedure I using 1-(4-iodophenyl)methanamine in STEP 1; Boc Deprotection; General Procedure II in STEP 2. MS: m/z Calc'd for C₂₀H₂₃INO₄[M+H]⁺483, Found 483. ¹H NMR (400 MHz, Methanol-d₄) δ 7.73-7.71 (m, 2H), 7.16-7.14 (m, 4H), 6.87-6.85 (m, 2H), 4.92-4.90 (m, 1H), 4.60 (s, 1H), 4.39-4.37 (m, 2H), 4.33-4.31 (m, 1H), 3.80 (s, 3H), 3.58-3.56 (m, 1H), 3.23-3.21 (m, 1H), 2.97-2.95 (m, 2H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 15% B to 45% B in 10 min; Wave Length: 254 nm/220 nm; RT1(min): 8.35.

### Synthetic Procedure of (2R,3S,4S)-4-hydroxy-2-[(4-methoxyphenyl)methyl]pyrrolidin-3-yl N-[(3R)-piperidin-3-ylmethyl]carbamate (V187929)

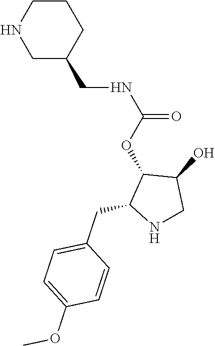 [SMILES: COc1ccc(C[C@H]2NC[C@H](O)[C@H]2OC(=O)NC[C@@H]2CCCNC2)cc1]

The title compound was prepared in 25.9% overall yield as a white semi-solid according to Substitution reaction for synthesis of Carbamate; General Procedure I using tert-butyl (3S)-3-(aminomethyl)piperidine-1-carboxylate in STEP 1; Boc Deprotection; General Procedure II in STEP 2. MS: m/z Calc'd for C₁₉H₂₉N₃O₄[M+H]⁺364, Found 364. ¹H NMR (400 MHz, Methanol-d₄) δ 7.28-7.20 (m, 2H), 6.97-6.89 (m, 2H), 4.94-4.92 (m, 1H), 4.38-4.36 (m, 1H), 4.17-4.15 (m, 1H), 3.80 (s, 3H), 3.58-3.56 (m, 1H), 3.37-3.35 (m, 2H), 3.26-3.06 (m, 4H), 3.04-2.88 (m, 2H), 2.79-2.65 (m, 1H), 2.02-1.91 (m, 3H), 1.83-1.68 (m, 1H), 1.40-1.26 (m, 1H).

Prep-HPLC purification conditions: Column: Welch Ultimate AQ-C18 50*250, 10 μm; Mobile Phase A: Water (0.05% TFA), Mobile Phase B: ACN; Flow rate: 60 mL/min; Gradient: 2% B to 40% B in 20 min; Wave Length: 254 nm/220 nm; RT1(min): 17.08.

### Synthetic Procedure of (2R,3S,4S)-4-hydroxy-2-[(4-methoxyphenyl)methyl]pyrrolidin-3-yl N-[(3S)-piperidin-3-ylmethyl]carbamate (V187930)

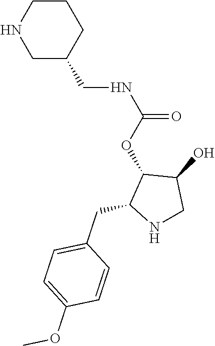 [SMILES: COc1ccc(C[C@H]2NC[C@H](O)[C@H]2OC(=O)NC[C@H]2CCCNC2)cc1]

The title compound was prepared in 25.9% overall yield as a white solid according to Substitution reaction for synthesis of Carbamate; General Procedure I using tert-butyl (3R)-3-(aminomethyl)piperidine-1-carboxylate in STEP 1; Boc Deprotection; General Procedure II in STEP 2. MS: m/z Calc'd for C₁₉H₂₉N₄O₄[M+H]⁺364, Found 364. ¹H NMR (400 MHz, Methanol-d₄) δ 7.24-7.22 (m, 2H), 6.93-6.91 (m, 2H), 4.96-4.94 (m, 1H), 4.39-4.35 (m, 1H), 4.17-4.15 (m, 1H), 3.80 (s, 3H), 3.59-3.57 (m, 1H), 3.43-3.35 (m, 2H), 3.26-3.07 (m, 4H), 3.04-2.87 (m, 2H), 2.78-2.66 (m, 1H), 2.08-1.90 (m, 3H), 1.83-1.68 (m, 1H), 1.33-1.31 (m, 1H).

Prep-HPLC purification conditions: Column: Welch Ultimate AQ-C18 50*250, 10 μm; Mobile Phase A: Water (0.05% TFA), Mobile Phase B: ACN; Flow rate: 60 mL/min; Gradient: 2% B to 25% B in 20 min; Wave Length: 254 nm/220 nm; RT1(min): 20.7.

### Synthetic Procedure of (2R,3S,4S)-4-hydroxy-2-[(4-methoxyphenyl)methyl]pyrrolidin-3-yl N-[2-(5-methyl-1,2-oxazol-3-yl)ethyl]carbamate (V187719)

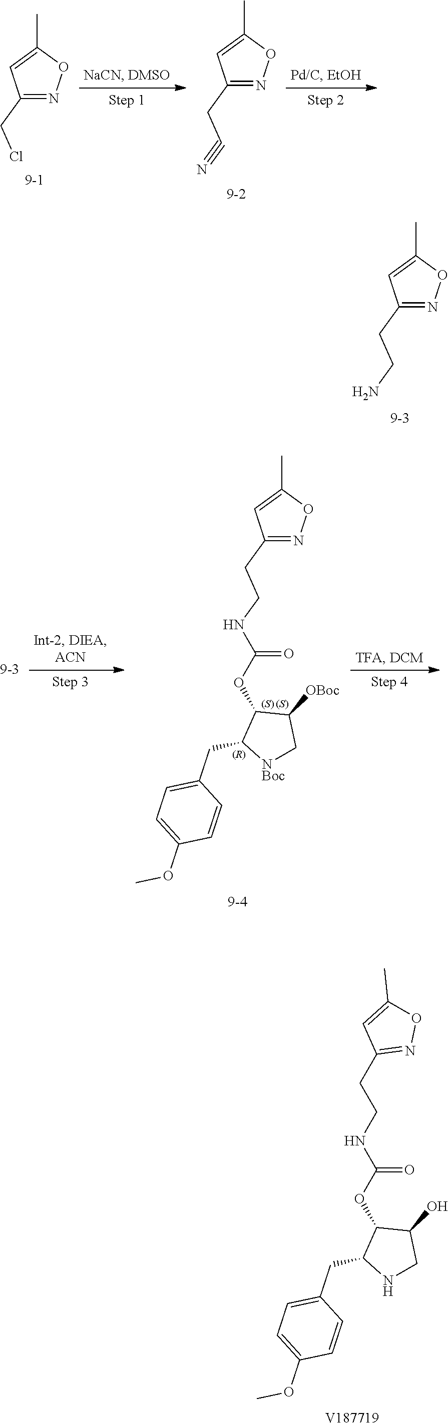

Step 1: To a stirred solution of 3-(chloromethyl)-5-methyl-1,2-oxazole (9-1, 1 g, 7.60 mmol, 1.0 eq.) in DMSO (10 mL) was added NaCN (745 mg, 15.202 mmol, 2.0 eq.) in portions at 0° C. The resulting mixture was stirred at 25° C. for overnight. LCMS showed the reaction was completed. The reaction was quenched with Water at 0° C. The aqueous layer was extracted with EtOAc (3×3 mL). Combined the organic phase, dried over anhydrous sodium sulfate. After filtration, the filtrate was concentrated under reduced pressure to afford 2-(5-methyl-1,2-oxazol-3-yl)acetonitrile (700 mg, 75.4%) as a light yellow solid which was used directly in the next step without further purification. MS: m/z: Calc'd for C₆H₆N₂O [M+H]⁺123. Found, 123.

Step 2: To a stirred solution of 2-(5-methyl-1,2-oxazol-3-yl)acetonitrile (9-2, 100 mg, 0.82 mmol, 1.0 eq.) in EtOH (5 mL) was added Pd/C (100 mg, 0.94 mmol, 1.2 eq.) in portions at room temperature under N₂. The reaction was placed under vacuum, sonicated and backfilled with H₂. The resulting mixture was stirred 1 h at 25° C. LCMS showed the reaction was completed. The resulting mixture was filtered, the filter cake was washed with EtOH (2×1 mL). The filtrate was concentrated under reduced pressure. The crude product was used in the next step directly without further purification. MS: m/z: Calc'd for C₆H₁₀N₂O [M+H]⁺127. Found, 127.

Step 3 and 4: The title compound was prepared in 24.0% overall yield as a light yellow semi-solid according to Substitution reaction for synthesis of Carbamate; General Procedure I using 2-(5-methyl-1,2-oxazol-3-yl)ethanamine (8-3) in STEP 3; Boc Deprotection; General Procedure II in STEP 4. MS: m/z Calc'd for C₁₉H₂₅N₃O₅ [M+H]⁺376. Found, 376. ¹H NMR (400 MHz, Methanol-d₄) δ 7.22 (d, J=8.3 Hz, 2H), 6.93 (d, J=8.3 Hz, 2H), 6.11 (s, 1H), 4.91 (d, J=3.5 Hz, 1H), 4.36 (d, J=4.3 Hz, 1H), 4.18-4.10 (m, J=7.8, 3.4 Hz, 1H), 3.80 (s, 3H), 3.61-3.40 (m, 3H), 3.21 (d, J=12.6 Hz, 1H), 3.07 (dd, J=14.1, 7.3 Hz, 1H), 2.98-2.80 (m, 3H), 2.38 (s, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 34% B to 55% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-[(4,5-dichloro-1H-imidazol-2-yl)methyl]carbamate (V187824)

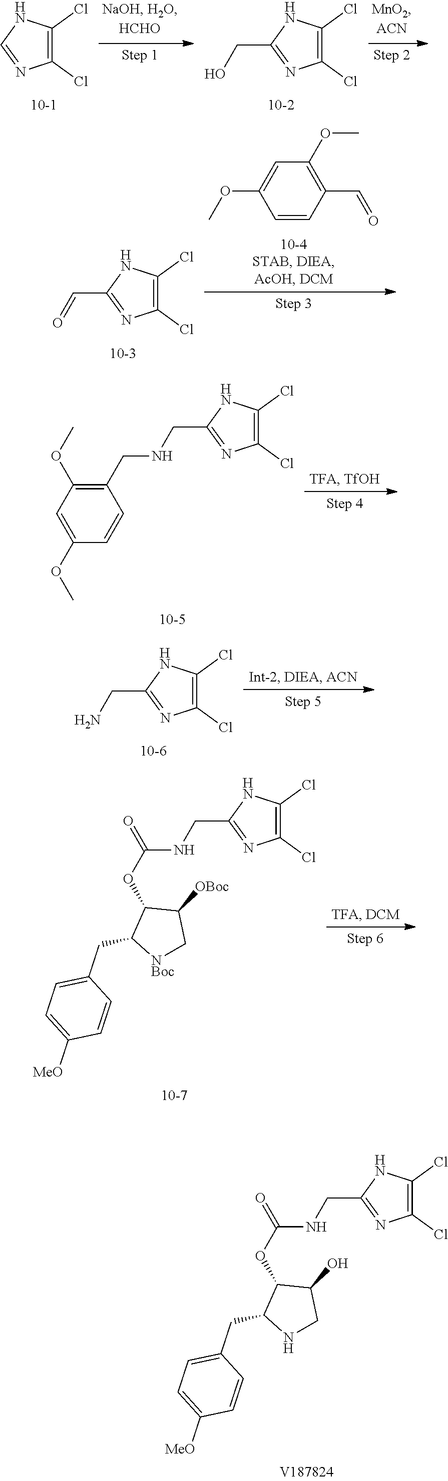

Step 1: To a stirred solution of 4,5-dichloro-1H-imidazole (10-1, 500 mg, 3.65 mmol, 1.0 eq.) in H₂O (3.6 mL) was added NaOH (146.02 mg, 3.65 mmol, 1.0 eq.) and HCHO (109.6 mg, 3.65 mmol, 1.0 eq.) in portions at room temperature. The resulting mixture was stirred at room temperature for 2 h. Desired product could be detected by LCMS. The PH was adjusted to 4 with HCl (2 mol/L) at 0° C. The solids were collected by filtration to give (4,5-dichloro-1H-imidazol-2-yl)methanol (470 mg, 77.1%) as a white solid, which was used in the next step directly without further purification. MS: m/z: Calc'd for C₄H₄Cl₂N₂O [M+H]⁺167. Found, 167.

Step 2: To a stirred solution of (4,5-dichloro-1H-imidazol-2-yl)methanol (10-2, 250 mg, 1.5 mmol, 1.0 eq.) in ACN (1 mL) was added MnO₂ (650.8 mg, 7.49 mmol, 5.0 eq.) in portions at room temperature. The resulting mixture was stirred over night at 60° C. After completion of reaction monitored by LCMS. The resulting mixture was filtered, the filter cake was washed with MeCN (1×3 mL). The filtrate was concentrated under reduced pressure to afford 4,5-dichloro-1H-imidazole-2-carbaldehyde (240 mg, 97.2%) as a yellow oil, which was used in the next step directly without further purification. MS: m/z: Calc'd for C₄H₂Cl₂N₂O [M+H]⁺165. Found, 165.

Step 3: To a stirred solution of 4,5-dichloro-1H-imidazole-2-carbaldehyde (10-3, 200 mg, 1.21 mmol, 1.0 eq.) and 2,4-dimethoxybenzaldehyde (10-4, 201.5 mg, 1.21 mmol, 1.0 eq.) in DCM (5 mL) was added DIEA (470 mg, 3.64 mmol, 3.0 eq.) and AcOH (291.2 mg, 4.85 mmol, 4.0 eq.) at room temperature. After 0.5 h STAB (770.8 mg, 3.64 mmol, 3.0 eq.) was added at 0° C. The resulting mixture was stirred over night at room temperature. After completion of reaction monitored by LCMS. The reaction was quenched with Water at 0° C. The aqueous layer was extracted with DCM (3×3 mL). The resulting mixture was concentrated under reduced pressure. The residue was purified by reversed-phase flash chromatography to afford [(4,5-dichloro-1H-imidazol-2-yl)methyl][(2,4-dimethoxyphenyl)methyl]amine (300 mg, 78.2%) as a white solid. MS: m/z: Calc'd for C₁₃H₁₅Cl₂N₃O₂ [M+H]⁺316. Found, 316.

Step 4: To a stirred solution of [(4,5-dichloro-1H-imidazol-2-yl)methyl][(2,4-dimethoxyphenyl)methyl]amine (10-5, 270 mg, 0.854 mmol, 1 eq.) in TFA (3 mL) was added trifluoromethanesulfonic acid (0.3 mL) at 0° C. The resulting mixture was stirred at room temperature for 2 h. The resulting mixture was concentrated under reduced pressure. The crude product was used in the next step directly without further purification. MS: m/z: Calc'd for C₄H₅Cl₂N₃[M+H]⁺166. Found, 166.

### Step 5 and 6

The title compound was prepared in 31.2% overall yield as a white solid according to Substitution reaction for synthesis of Carbamate; General Procedure I using 1-(4,5-dichloro-1H-imidazol-2-yl)methanamine (10-6) in STEP 5; Boc Deprotection; General Procedure II in STEP 6. MS: m/z Calc'd for C₁₇H₂₀Cl₂N₄O₄ [M+H]⁺415. Found, 415. ¹H NMR (400 MHz, Methanol-d₄) δ 7.25-7.09 (m, 2H), 6.90 (dd, J=8.4, 1.8 Hz, 2H), 4.95 (d, J=3.3 Hz, 1H), 4.42 (d, J=4.2 Hz, 1H), 4.31 (s, 2H), 4.18-4.14 (m, 1H), 3.79 (s, 3H), 3.62-3.58 (m, 1H), 3.22 (d, J=12.6 Hz, 1H), 3.11 (dd, J=14.1, 7.3 Hz, 1H), 3.04-2.92 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 5% B to 35% B in 10 min; Wave Length: 254 nm/220 nm; RT1(min): 8.57.

### Synthetic Procedure of (2R,3S,4S)-4-hydroxy-2-[(4-methoxyphenyl)methyl]pyrrolidin-3-yl N-[2-(5-methyl-1H-imidazol-4-yl)ethyl]carbamate (V187823)

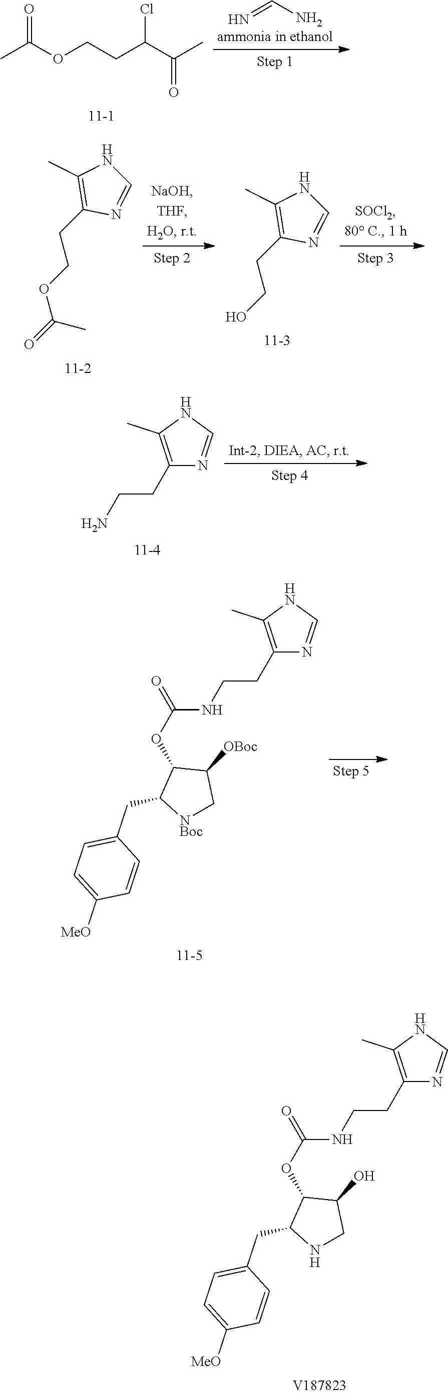

Step 1: To a stirred mixture of 3-chloro-4-oxopentyl acetate (11-1, 1 g, 5.59 mmol, 1 equiv) in EtOH (10 mL) was added formamidine (0.25 g, 5.59 mmol, 1 equiv) at room temperature. The resulting mixture was stirred at 80° C. for overnight. The resulting mixture was concentrated under reduced pressure. The residue was purified by reversed-phase flash chromatography to afford 2-(5-methyl-1H-imidazol-4-yl)ethyl acetate (500 mg, 53.1%) as a yellow oil. MS: m/z: Calc'd for C₈H₁₂N₂O₂ [M+H]⁺169. found 169.

Step 2: To a stirred mixture of 2-(5-methyl-1H-imidazol-4-yl)ethyl acetate (11-2, 300 mg, 1.784 mmol, 1 equiv) in THF (7.5 mL) and water (2.5 mL) was added NaOH (214.1 mg, 5.35 mmol, 3 equiv) at room temperature. The resulting mixture was stirred at room temperature for overnight. Upon completion, the mixture was concentrated, the residue was purified by reversed-phase flash chromatography to afford 2-(5-methyl-1H-imidazol-4-yl)ethanol (100 mg, 44.4%) as a yellow oil. MS: m/z: Calc'd for C₆H₁₀N₂O [M+H]⁺127. found 127.

Step 3: To a stirred mixture of 2-(5-methyl-1H-imidazol-4-yl)ethanol (11-3, 300 mg, 2.38 mmol, 1 equiv) was added SOCl₂ (10 mL) at room temperature under. The resulting mixture was stirred at 80° C. for overnight. The resulting mixture was concentrated under reduced pressure. The residue was added Ammonia at room temperature under nitrogen atmosphere. The resulting mixture was stirred at 80° C. for overnight. Upon completion, the mixture was concentrated in vacuum. The crude was used in the next step directly without further purification. MS: m/z: Calc'd for C₆H₁₁N₃ [M+H]⁺126. found 126.

### Step 4 and 5

The title compound was prepared in 21.5% overall yield as a yellow solid according to Substitution reaction for synthesis of Carbamate; General Procedure I using 4-methylhistamine (11-4) in STEP 4; Boc Deprotection; General Procedure II in STEP 5. MS: m/z Calc'd for C₁₉H₂₆N₄O₄ [M+H]⁺375. found 375. ¹H NMR (400 MHz, Methanol-d₄) δ 8.71 (s, 1H), 7.22 (d, J=8.2 Hz, 2H), 6.92 (d, J=8.3 Hz, 2H), 4.94 (d, J=3.5 Hz, 1H), 4.33 (d, J=4.2 Hz, 1H), 4.14-4.02 (m, 1H), 3.79 (s, 3H), 3.56 (dd, J=12.7, 4.3 Hz, 1H), 3.43-3.32 (m, 2H), 3.20 (d, J=12.7 Hz, 1H), 3.07 (dd, J=14.2, 6.7 Hz, 1H), 2.92-2.83 (m, 3H), 2.32 (s, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 9.13.

### Synthetic Procedure of (2R,3S,4S)-4-hydroxy-2-[(4-methoxyphenyl)methyl]pyrrolidin-3-yl N-[2-(1,3-thiazol-2-ylamino)ethyl]carbamate (V187826)

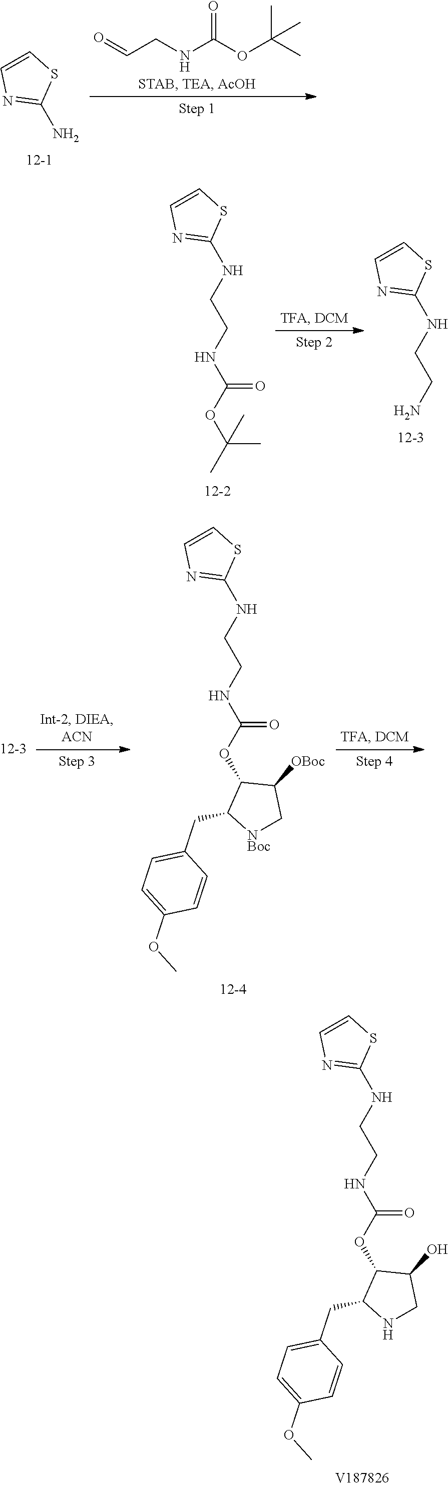

Step 1: To a stirred solution of aminothiazole (500 mg, 4.99 mmol, 1 eq.) and tert-butyl N-(2-oxoethyl)carbamate (1.6 g, 9.99 mmol, 2 eq.) in MeOH (100 mL) was treated with AcOH (10 mL) for 1 h at 0° C. under nitrogen atmosphere followed by the addition of (2-methylpyridin-1-ium-1-yl)boranuide (2.1 g, 19.97 mmol, 4 eq.) at 0° C. The resulting mixture was stirred at room temperature for 1 h. After completion of reaction monitored by LCMS. The reaction was quenched with Water at 0° C. The resulting mixture was concentrated under reduced pressure. The residue was purified by flash. This resulted in tert-butyl N-[2-(1,3-thiazol-2-ylamino)ethyl]carbamate (190 mg, 15.6%) as a light yellow oil. MS: m/z: Calc'd for C₁₀H₁₇N₃O₂S [M+H]⁺ 244. found [M+H]⁺ 244.

Step 2: To a stirred solution of tert-butyl N-[2-(1,3-thiazol-2-ylamino)ethyl]carbamate (12-2, 180 mg, 0.74 mmol) in DCM (5 mL) was added TFA (1 mL). The resulting mixture was stirred at room temperature for 1 h. After completion of reaction monitored by LCMS, concentrated, the crude product was used in the next step directly without further purification. MS: m/z: Calc'd for C₅H₉N₃S [M+H]⁺ 144. found 144.

Step 3 and 4: The title compound was prepared in 32.4% overall yield as an off-white solid according to Substitution reaction for synthesis of Carbamate; General Procedure I using N1-(1,3-thiazol-2-yl)ethane-1,2-diamine (12-3) in STEP 3; Boc Deprotection; General Procedure II in STEP 4. MS: m/z Calc'd for C₁₈H₂₄N₄O₄S [M+H]⁺393. found 393. ¹H NMR (400 MHz, Methanol-d₄) δ 7.28-7.18 (m, 3H), 6.97-6.88 (m, 2H), 6.86 (d, J=4.2 Hz, 1H), 5.00-4.95 (m, 1H), 4.40-4.34 (m, 1H), 4.18-4.14 (m, 1H), 3.79 (s, 3H), 3.59-3.44 (m, 5H), 3.21 (d, J=12.6 Hz, 1H), 3.09 (dd, J=14.2, 6.8 Hz, 1H), 2.94 (dd, J=14.2, 8.7 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-[2-(1-methylpyrazol-3-yl)ethyl]carbamate (V187847)

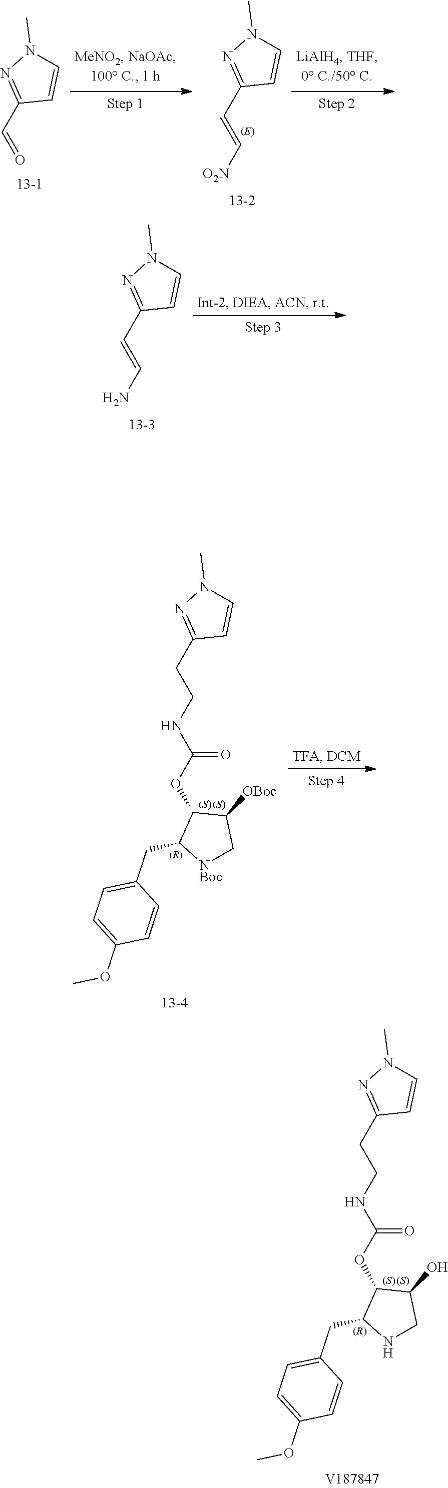

### Step 1

To a stirred mixture of 1-methylpyrazole-3-carbaldehyde (1 g, 9.10 mmol, 1 eq.) in nitromethane (10 mL) was added ammonium acetate (1.05 g, 13.62 mmol, 1.5 eq.) at room temperature. The resulting mixture was stirred for overnight at 110° C. The resulting mixture was concentrated under reduced pressure. The residue was purified by silica gel column chromatography, eluted with PE/EA (3:1) to afford 1-methyl-3-[(E)-2-nitroethenyl]pyrazole (150 mg, 10.8%) as a yellow oil. MS: m/z: Calc'd for C₆H₇N₃O₂[M+H]⁺ 154. found 154.

Step 2: To a stirred mixture of 1-methyl-3-[(E)-2-nitroethenyl]pyrazole (100 mg, 0.65 mmol, 1 eq.) in THF (5 mL) was added LiAlH₄ (1 mL, 0.98 mmol, 1.5 eq.) at 0° C. The resulting mixture was stirred at 50° C. for overnight. The reaction was quenched with water/EA at 0° C. The precipitated solids were collected by filtration and washed with EtOAc (2×20 mL). The resulting mixture was concentrated under vacuum to afford crude product as a yellow oil. MS: m/z: Calc'd for C₆H₁₁N₃ [M+H]⁺126. found 126.

Step 3 and 4: The title compound was prepared in 6.6% overall yield as a yellow solid according to Substitution reaction for synthesis of Carbamate; General Procedure I using 2-(1-methylpyrazol-3-yl)ethanamine (13-3) in STEP 3; Boc Deprotection; General Procedure II in STEP 4. MS: m/z Calc'd for C₁₉H₂₆N₄O₄ [M+H]⁺375. found 375. ¹H NMR (400 MHz, Methanol-d₄) δ 7.49 (d, J=2.2 Hz, 1H), 7.25-7.17 (m, 2H), 6.96-6.88 (m, 2H), 6.16 (d, J=2.2 Hz, 1H), 4.96-4.91 (m, 1H), 4.37 (d, J=4.3 Hz, 1H), 4.13-4.02 (m, 1H), 3.81 (d, J=13.0 Hz, 6H), 3.64-3.35 (m, 3H), 3.27-3.02 (m, 2H), 3.02-2.74 (m, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 28% B in 10 min; Wave Length: 254 nm/220 nm; RT1(min): 8.6.

### Synthetic Procedure of (2R,3S,4S)-4-hydroxy-2-[(4-methoxyphenyl)methyl]pyrrolidin-3-yl N-{2-[(3S)-pyrrolidin-3-yl]ethyl}carbamate (V187878)

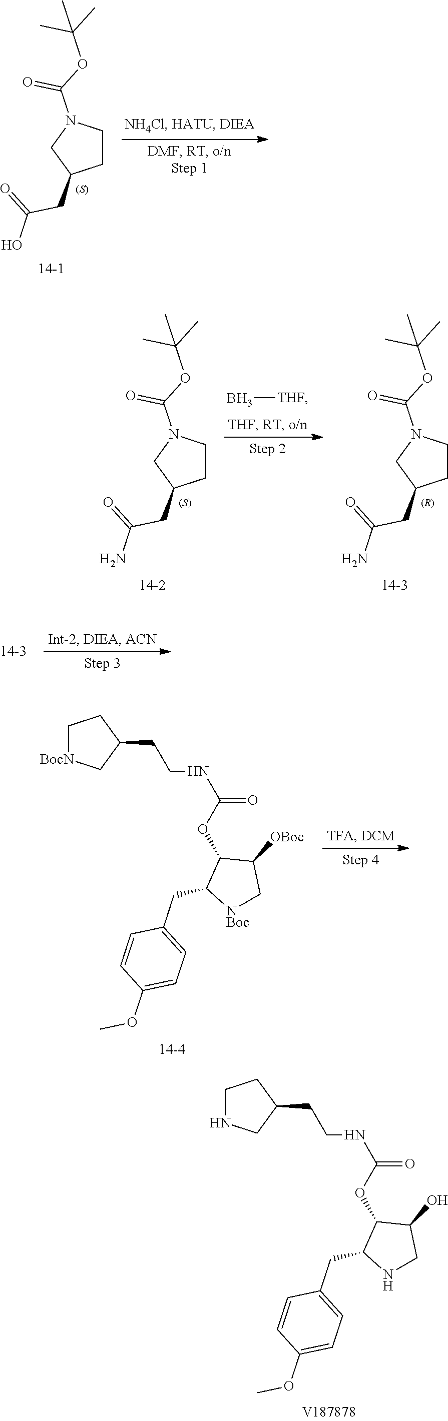

Step 1: To a stirred solution of [(3S)-1-(tert-butoxycarbonyl)pyrrolidin-3-yl]acetic acid (14-1, 500 mg, 2.18 mmol, 1.0 eq.) and NH₄Cl (583.3 mg, 10.91 mmol, 5.0 eq.) in DMF (10 mL) was added HATU (1658.4 mg, 4.36 mmol, 2.0 eq.) and DIEA (1127.4 mg, 8.72 mmol, 4.0 eq.) in portions at room temperature. The resulting mixture was stirred at 25° C. for overnight. The resulting mixture was extracted with EtOAc (3×10 mL). The combined organic layers were washed with water (3×10 mL), dried over anhydrous Na2SO4. After filtration, the filtrate was concentrated under reduced pressure. The residue was purified by reversed-phase flash chromatography to afford tert-butyl (3S)-3-(carbamoylmethyl)pyrrolidine-1-carboxylate (400 mg, 80.3%) as a light yellow oil. MS: m/z: Calc'd for C₁₁H₂₀N₂O₃ [M+H+22]⁺251. Found, 251.

Step 2: To a stirred solution of tert-butyl (3S)-3-(carbamoylmethyl)pyrrolidine-1-carboxylate (14-2, 150 mg, 0.66 mmol, 1.0 eq.) in THF (5 mL) was added BH₃-THF (3.29 mL, 3.285 mmol, 5.0 eq.) in portions at −78° C. The resulting mixture was stirred at −78° C. for 1 h under a nitrogen atmosphere. Desired product could be detected by LCMS. The reaction was quenched with MeOH at 0° C. The resulting mixture was concentrated under reduced pressure. The resulting mixture was used in the next step directly without further purification. MS: m/z: Calc'd for C₁₁H₂₂N₂O₂ [M+H]⁺215. Found, 215.

Step 3 and 4: The title compound was prepared in 35.8% overall yield as a yellow semi-solid according to Substitution reaction for synthesis of Carbamate; General Procedure I using tert-butyl (3R)-3-(2-aminoethyl)pyrrolidine-1-carboxylate (14-3) in STEP 3; Boc Deprotection; General Procedure II in STEP 4. MS: m/z Calc'd for C₁₉H₂₉N₃O₄ [M+H]⁺364. Found, 364. ¹H NMR (400 MHz, Methanol-d₄) δ 7.28-7.21 (m, 2H), 6.96-6.90 (m, 2H), 4.95 (d, J=3.5 Hz, 1H), 4.38 (d, J=4.1 Hz, 1H), 4.19-4.15 (m, J=7.8, 3.5 Hz, 1H), 3.80 (s, 3H), 3.57 (dd, J=12.6, 4.3 Hz, 1H), 3.50 (dd, J=11.5, 7.6 Hz, 1H), 3.44-3.38 (m, J=12.1, 8.5, 3.9 Hz, 1H), 3.27 (d, J=8.6 Hz, 1H), 3.26-3.17 (m, 3H), 3.11 (dd, J=14.1, 7.1 Hz, 1H), 2.98 (dd, J=14.1, 8.5 Hz, 1H), 2.88 (dd, J=11.6, 9.1 Hz, 1H), 2.44-2.21 (m, 2H), 1.81-1.61 (m, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 8.77.

### Synthetic Procedure of (2R,3S,4S)-4-hydroxy-2-[(4-methoxyphenyl)methyl]pyrrolidin-3-yl N-{2-[(1-aminocyclopropyl)formamido]ethyl}carbamate (V187919)

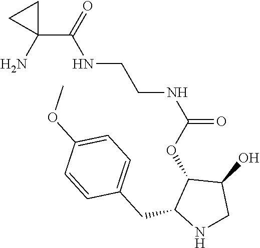 [SMILES: COc1ccc(C[C@H]2NC[C@H](O)[C@H]2OC(=O)NCCNC(=O)C2(N)CC2)cc1]

The title compound was prepared in 63.2% overall yield as a white solid according to Substitution reaction for synthesis of Carbamate; General Procedure I using tert-butyl N-{1-[(2-aminoethyl)carbamoyl]cyclopropyl}carbamate in STEP 1; Boc Deprotection; General Procedure II in STEP 2. MS: m/z Calc'd for C₁₉H₂₈N₄O₅ [M+H]⁺393. found 393. ¹H NMR (400 MHz, Methanol-d₄) δ 7.27-7.21 (m, 2H), 6.96-6.90 (m, 2H), 4.98-4.92 (m, 1H), 4.39 (d, J=4.2 Hz, 1H), 4.19-4.12 (m, 1H), 3.79 (s, 3H), 3.58 (dd, J=12.7, 4.3 Hz, 1H), 3.36 (s, 2H), 3.28-3.22 (m, 2H), 3.19 (s, 1H), 3.11 (dd, J=14.2, 7.0 Hz, 1H), 2.97 (dd, J=14.2, 8.7 Hz, 1H), 1.57-1.43 (m, 2H), 1.43-1.30 (m, 2H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 30% B in 20 min; Wave Length: 254 nm/220 nm; RT1(min): 17.13.

### Synthetic Procedure of (2R,3S,4S)-4-hydroxy-2-[(4-methoxyphenyl)methyl]pyrrolidin-3-yl N-[2-(3-cyanopyridin-4-yl)ethyl]carbamate (V187940)

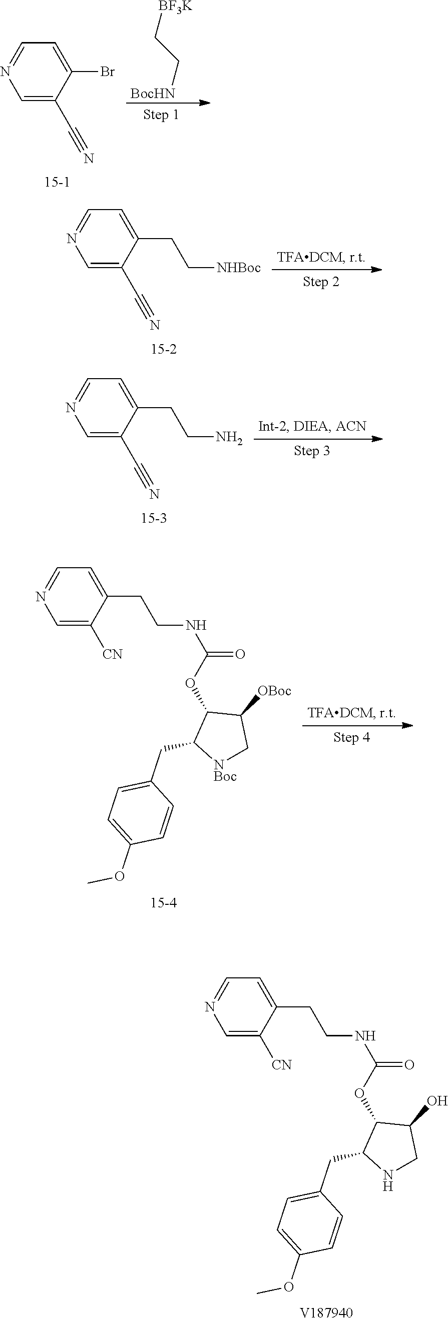

Step 1: To a stirred mixture of 4-bromopyridine-3-carbonitrile (15-1, 500 mg, 2.73 mmol, 1 eq.) and tert-butyl N-[2-(trifluoro-lambda4-boranyl)ethyl]carbamate potassium (1029.0 mg, 4.09 mmol, 1.5 eq.) in Toluene (7.5 mL) and H₂O (2.5 mL) was added Pd(dppf)Cl₂ (399.8 mg, 0.54 mmol, 0.2 eq.) and Cs₂CO₃ (2670.5 mg, 8.19 mmol, 3 eq.) at room temperature under nitrogen atmosphere. The resulting mixture was stirred at 90° C. for overnight under nitrogen atmosphere. The resulting mixture was concentrated under reduced pressure. The residue was dissolved in MeCN (5 mL) and purified by reversed-phase flash chromatography to afford tert-butyl N-[2-(3-cyanopyridin-4-yl)ethyl]carbamate (500 mg, 74.0%) as a yellow solid.

Step 2: To a stirred mixture of tert-butyl N-[2-(3-cyanopyridin-4-yl)ethyl]carbamate (15-2, 230 mg, 0.93 mmol) in DCM (5 mL) was added TFA (1 mL) at room temperature. The resulting mixture was stirred at room temperature for 2 h. The resulting mixture was concentrated under reduced pressure. This resulted in 4-(2-aminoethyl)pyridine-3-carbonitrile (120 mg, 87.6%) as a yellow oil. MS: m/z: Calc'd for C₈H₉N₃[M+H]⁺148. found 148.

Step 3 and 4: The title compound was prepared in 13.5% overall yield as a yellow solid according to Substitution reaction for synthesis of Carbamate; General Procedure I using 4-(2-aminoethyl)pyridine-3-carbonitrile (15-3) in STEP 3; Boc Deprotection; General Procedure II in STEP 4. MS: m/z Calc'd for C₂₁H₂₄N₄O₄ [M+H]⁺397. found 397. ¹H NMR (400 MHz, Methanol-d₄) δ 8.85 (s, 1H), 8.71 (d, J=5.2 Hz, 1H), 7.57 (d, J=5.2 Hz, 1H), 7.27-7.16 (m, 2H), 6.96-6.88 (m, 2H), 4.89 (s, 1H), 4.42-4.36 (m, 1H), 4.13-4.02 (m, 1H), 3.79 (s, 3H), 3.71-3.44 (m, 3H), 3.20 (d, J=12.7 Hz, 1H), 3.16-3.03 (m, 3H), 2.92 (dd, J=14.2, 8.6 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 30% B in 10 min; Wave Length: 254 nm/220 nm; RT1(min): 8.82.

### Synthetic Procedure of (2R,3S,4S)-4-hydroxy-2-[(4-methoxyphenyl)methyl]pyrrolidin-3-yl N-[2-(4-cyano-1H-pyrazol-3-yl)ethyl]carbamate (V187942)

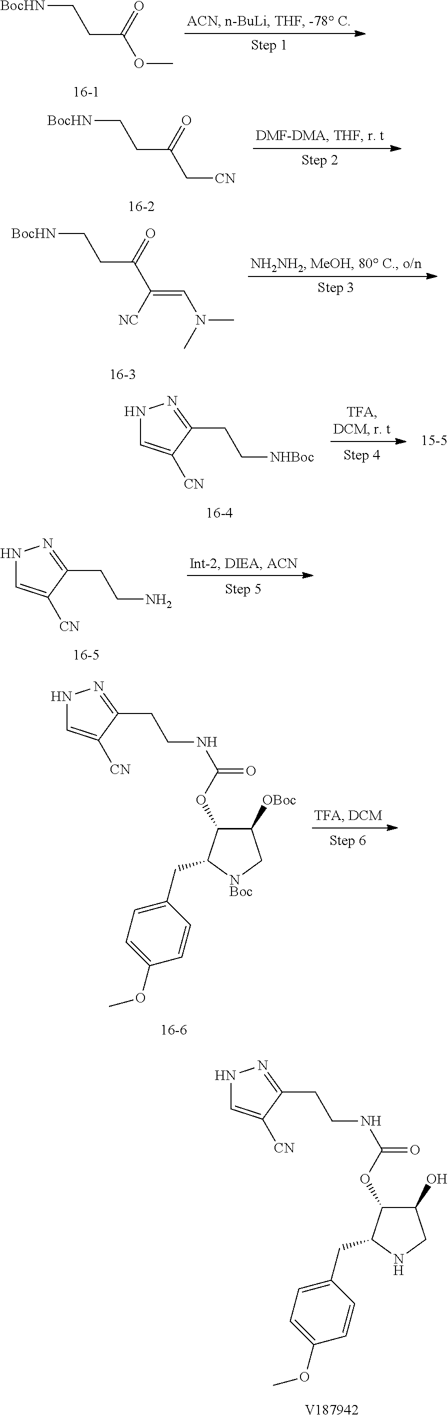

Step 1: A solution of n-BuLi (0.79 g, 12.3 mmol, 2.5 equiv) in THF (15 mL) was treated with ACN (0.50 g, 12.3 mmol, 2.5 equiv) for 1 h at −78° C. under nitrogen atmosphere followed by the addition of methyl 3-[(tert-butoxycarbonyl)amino]propanoate (16-1, 1 g, 4.92 mmol, 1 equiv) at −78° C. The resulting mixture was stirred at −50° C. for 1 h under nitrogen atmosphere. The reaction was quenched by the addition of sat. NH₄Cl (aq.) (10 mL) at 0° C. The resulting mixture was quenched with water and extracted with EA. The extracts were washed with brine, dried over anhydrous sodium sulfate, filtered and concentrated. The residue was purified by silica gel column chromatography, eluted with PE/EA (3:1) to afford tert-butyl N-(4-cyano-3-oxobutyl)carbamate (500 mg, 47.8%) as a brown oil.

Step 2: To a stirred mixture of tert-butyl N-[2-(3-cyanopyridin-4-yl)ethyl]carbamate (16-2, 230 mg, 0.93 mmol) in DCM (5 mL) was added TFA (1 mL) at room temperature. The resulting mixture was stirred at room temperature for 2 h. The resulting mixture was concentrated under reduced pressure. This resulted in 4-(2-aminoethyl)pyridine-3-carbonitrile (120 mg, 87.6%) as a yellow oil. MS: m/z: Calc'd for C₈H₉N₃[M+H]⁺148. found 148.

Step 3: To a stirred solution/mixture of tert-butyl N-[(4E)-4-cyano-4-[(dimethylamino)methylidene]-3-oxobutyl]carbamate (16-3, 300 mg, 1.12 mmol, 1 eq.) in MeOH (5 mL) was added hydrazine hydrate (168.5 mg, 3.36 mmol, 3 eq.) at room temperature under nitrogen atmosphere. The resulting mixture was stirred at 80° C. for overnight under nitrogen atmosphere. The resulting mixture was concentrated under reduced pressure. The residue was dissolved in DMSO (5 mL). The residue was purified by reversed-phase flash chromatography to afford tert-butyl N-[2-(4-cyano-1H-pyrazol-3-yl)ethyl]carbamate (200 mg, 75.4%) as a yellow oil. MS: m/z: Calc'd for C₁₁H₁₆N₄O₂ [M+H+22]⁺259. found 259.

Step 4: To a stirred mixture of tert-butyl N-[2-(4-cyano-1H-pyrazol-3-yl)ethyl]carbamate (16-4, 300 mg, 1.27 mmol) in DCM (5 mL) was added TFA (1 mL, 13.46 mmol, 10.6 eq) at room temperature. The resulting mixture was stirred at room temperature for 3 h. The resulting mixture was concentrated under vacuum. This resulted in 3-(2-aminoethyl)-1H-pyrazole-4-carbonitrile (160 mg, 92.5%) as a yellow oil. MS: m/z: Calc'd for C₆H₈N₄ [M+H]⁺137. found 137.

### Step 5 and 6

The title compound was prepared in 24.5% overall yield as a white solid according to Substitution reaction for synthesis of Carbamate; General Procedure I using 3-(2-aminoethyl)-1H-pyrazole-4-carbonitrile (16-5) in STEP 5; Boc Deprotection; General Procedure II in STEP 6. MS: m/z Calc'd for C₁₉H₂₃N₅O₄ [M+H]⁺386. found 386. ¹H NMR (400 MHz, Methanol-d₄) δ 8.07 (s, 1H), 7.21 (d, J=8.5 Hz, 2H), 6.96-6.88 (m, 2H), 4.90 (s, 1H), 4.41 (d, J=4.2 Hz, 1H), 4.13-4.02 (m, 1H), 3.79 (s, 3H), 3.69-3.50 (m, 2H), 3.45-3.32 (m, 1H), 3.20 (d, J=12.6 Hz, 1H), 3.14-2.91 (m, 4H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 32% B in 10 min; Wave Length: 254 nm/220 nm; RT1(min): 8.83.

### Synthetic Procedure of (2R,3S,4S)-4-hydroxy-2-({4-[1-(1H-pyrazol-4-ylmethyl)-1,2,3-triazol-4-yl]phenyl}methyl)pyrrolidin-3-yl N-[2-(pyridin-4-yl)ethyl]carbamate (V187948)

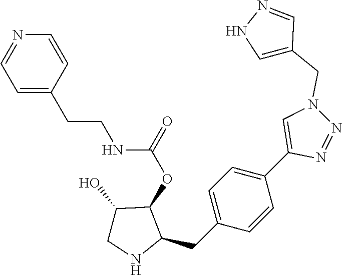 [SMILES: O=C(NCCc1ccncc1)O[C@@H]1[C@@H](O)CN[C@@H]1Cc1ccc(-c2cn(Cc3cn[nH]c3)nn2)cc1]

The title compound was prepared in 30.4% overall yield as a light green solid according to Substitution reaction for synthesis of Carbamate; General Procedure I using tert-butyl (2R,3S,4S)-4-[(tert-butoxycarbonyl)oxy]-2-[(4-ethynylphenyl)methyl]-3-[(4-nitrophenoxycarbonyl)oxy]pyrrolidine-1-carboxylate (2-7) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 2-(pyridin-4-yl)ethan-1-amine in STEP 1; Cyclization reaction for synthesis of Substituted triazoles; General Procedure III using 4-(azidomethyl)-1H-pyrazole in STEP 2; Boc Deprotection; General Procedure II in STEP 3. MS: m/z Calc'd for C₂₅H₂₈N₈O₃ [M+H]⁺489. found 489. ¹H NMR (400 MHz, Methanol-d₄) δ 8.68 (d, J=94.4 Hz, 2H), 8.32 (s, 1H), 8.02 (s, 2H), 7.81 (d, J=8.0 Hz, 4H), 7.36 (d, J=8.0 Hz, 2H), 5.61 (s, 2H), 5.05 (s, 1H), 4.35 (d, J=4.1 Hz, 1H), 4.25-4.16 (m, 1H), 3.67-3.51 (m, 3H), 3.26-3.10 (m, 4H), 3.04 (dd, J=14.1, 8.4 Hz, 1H).

Prep-HPLC purification conditions: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 8.77.

### Synthetic Procedure of (2R,3S,4S)-2-({4-[1-(4-chlorophenyl)-1,2,3-triazol-4-yl]phenyl}methyl)-4-hydroxypyrrolidin-3-yl N-[2-(pyridin-4-yl)ethyl]carbamate (V187950)

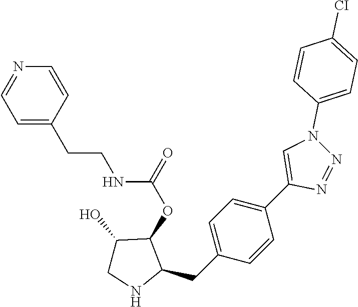

The title compound was prepared in 36.9% overall yield as a white solid according to Substitution reaction for synthesis of Carbamate; General Procedure I using tert-butyl (2R,3S,4S)-4-[(tert-butoxycarbonyl)oxy]-2-[(4-ethynylphenyl)methyl]-3-[(4-nitrophenoxycarbonyl)oxy]pyrrolidine-1-carboxylate (2-7) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 2-(pyridin-4-yl)ethan-1-amine in STEP 1; Cyclization reaction for synthesis of Substituted triazoles; General Procedure III using 1-azido-4-chlorobenzene in STEP 2; Boc Deprotection; General Procedure II in STEP 3. MS: m/z C₂₇H₂₇ClN₆O₃[M+H]⁺519. found 519. ¹H NMR (400 MHz, Methanol-d₄) δ 8.68-8.97 (s, 1H), 8.69 (s, 2H), 7.98-7.92 (m, 4H), 7.85 (s, 2H), 7.65 (dd, J=8.7, 1.6 Hz, 2H), 7.43 (d, J=8.1 Hz, 2H), 4.96 (s, 1H), 4.37 (d, J=4.0 Hz, 1H), 4.26 (s, 1H), 3.65-3.54 (m, 3H), 3.24 (d, J=13.1 Hz, 1H), 3.18-3.02 (m, 4H).

Prep-HPLC purification conditions: Xselect CSH Prep C18 Column, 30*250 mm, 10 μm; Mobile Phase A: Water (0.05% TFA), Mobile Phase B: ACN; Flow rate: 60 mL/min; Gradient: 11% B to 41% B in 10 min; Wave Length: 254 nm/220 nm; RT1(min): 8.75.

### Synthetic Procedure of (2R,3S,4S)-4-hydroxy-2-[(4-methoxyphenyl)methyl]pyrrolidin-3-yl N-{2-[(1-aminocyclopropyl)formamido]ethyl}carbamate (V187979)

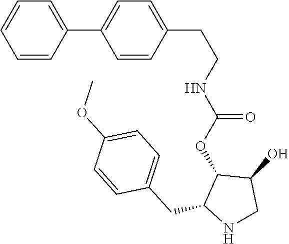 [SMILES: COc1ccc(C[C@H]2NC[C@H](O)[C@H]2OC(=O)NCCc2ccc(-c3ccccc3)cc2)cc1]

The title compound was prepared in 50.3% overall yield as a white solid according to Substitution reaction for synthesis of Carbamate; General Procedure I using 2-{[1,1′-biphenyl]-4-yl}ethanamine in STEP 1; Boc Deprotection; General Procedure II in STEP 2. MS: m/z Calc'd for C₂₇H₃₀N₂O₄[M+H]⁺447. found 447. ¹H NMR (400 MHz, Methanol-d₄) δ 7.60-7.51 (m, 4H), 7.41 (dd, J=8.5, 6.8 Hz, 2H), 7.38-7.29 (m, 3H), 7.19-7.14 (m, 2H), 6.91-6.84 (m, 2H), 4.93 (d, J=3.4 Hz, 1H), 4.36-4.30 (m, 1H), 4.15-4.06 (m, 1H), 3.75 (d, J=0.7 Hz, 3H), 3.59-3.39 (m, 3H), 3.18 (d, J=12.6 Hz, 1H), 3.03 (dd, J=14.2, 7.0 Hz, 1H), 2.99-2.81 (m, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 20% B to 50% B in 10 min; Wave Length: 254 nm/220 nm; RT1(min): 8.2.

### Synthetic Procedure of (2R,3S,4S)-4-hydroxy-2-[(4-methoxyphenyl)methyl]pyrrolidin-3-yl N-(pyridin-4-ylmethyl)carbamate (V187987)

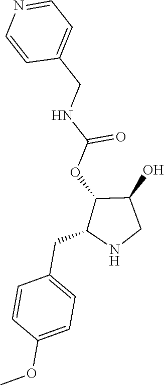 [SMILES: COc1ccc(C[C@H]2NC[C@H](O)[C@H]2OC(=O)NCc2ccncc2)cc1]

The title compound was prepared in 30.8% overall yield as a yellow solid according to Substitution reaction for synthesis of Carbamate; General Procedure I using 4-pyridylmethanamine in STEP 1; Boc Deprotection; General Procedure II in STEP 2. MS: m/z Calc'd for C₁₉H₂₃N₃O₄ [M+H]⁺358. found 358. ¹H NMR (400 MHz, Methanol-d₄) δ 8.78 (s, 2H), 7.93 (s, 2H), 7.28-7.21 (m, 2H), 6.92 (d, J=8.5 Hz, 2H), 4.87 (s, 2H), 4.63 (s, 1H), 4.46-4.40 (m, 1H), 4.21-4.10 (m, 1H), 3.79 (d, J=2.9 Hz, 3H), 3.62-3.50 (m, 1H), 3.24 (d, J=12.6 Hz, 1H), 3.14 (dd, J=14.2, 7.3 Hz, 1H), 3.08-2.98 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 7.53.

### Synthetic Procedure of (2R,3S,4S)-4-hydroxy-2-[(4-methoxyphenyl)methyl]pyrrolidin-3-yl N-[(2-aminopyridin-4-yl)methyl]carbamate (V187988)

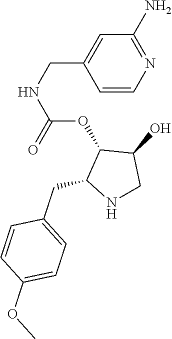 [SMILES: COc1ccc(C[C@H]2NC[C@H](O)[C@H]2OC(=O)NCc2ccnc(N)c2)cc1]

The title compound was prepared in 51.8% overall yield as a white solid according to Substitution reaction for synthesis of Carbamate; General Procedure I using tert-butyl N-[4-(aminomethyl)pyridin-2-yl]carbamate in STEP 1; Boc Deprotection; General Procedure II in STEP 2. MS: m/z Calc'd for C₁₉H₂₄N₄O₄ [M+H]⁺373. found 373. ¹H NMR (400 MHz, Methanol-d₄) δ 7.83 (d, J=7.0 Hz, 1H), 7.25 (d, J=8.5 Hz, 2H), 6.92 (d, J=8.3 Hz, 3H), 6.85 (d, J=6.7 Hz, 1H), 5.00 (s, 1H), 4.40 (d, J=15.7 Hz, 3H), 4.21 (s, 1H), 3.80 (s, 3H), 3.60 (d, J=11.8 Hz, 1H), 3.25 (d, J=12.8 Hz, 1H), 3.19-3.09 (m, 1H), 3.06-2.95 (m, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 30% B in 20 min; Wave Length: 254 nm/220 nm; RT1(min): 18.87.

### Synthetic Procedure of (2R,3S,4S)-4-hydroxy-2-[(4-methoxyphenyl)methyl]pyrrolidin-3-yl N-(carbamoylmethyl)carbamate (V188002)

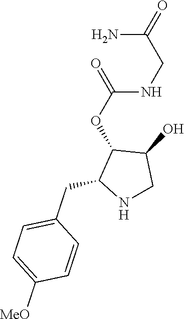 [SMILES: COc1ccc(C[C@H]2NC[C@H](O)[C@H]2OC(=O)NCC(N)=O)cc1]

The title compound was prepared in 12.0% overall yield as a white solid according to Substitution reaction for synthesis of Carbamate; General Procedure I using glycinamide in STEP 1; Boc Deprotection; General Procedure II in STEP 2. MS: m/z Calc'd for C₁₅H₂₁N₃O₅ [M+H]⁺324. Found, 324. ¹H NMR (400 MHz, Methanol-d₄) δ 7.30-7.22 (m, 2H), 6.98-6.90 (m, 2H), 4.96 (d, J=2.9 Hz, 1H), 4.44 (d, J=4.4 Hz, 1H), 4.20-4.11 (m, 1H), 3.85 (d, J=2.9 Hz, 2H), 3.80 (s, 3H), 3.62 (dd, J=12.6, 4.4 Hz, 1H), 3.22 (d, J=12.6 Hz, 1H), 3.15 (dd, J=14.2, 7.1 Hz, 1H), 2.99 (dd, J=14.2, 8.6 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 8.25.

### Synthetic Procedure of 1-{[(2R,3S,4S)-4-hydroxy-2-[(4-methoxyphenyl)methyl]pyrrolidin-3-yl]oxy}-N-[(methylcarbamoyl)amino]formamide (V188003)

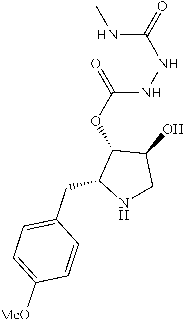 [SMILES: CNC(=O)NNC(=O)O[C@@H]1[C@@H](O)CN[C@@H]1Cc1ccc(OC)cc1]

The title compound was prepared in 42.5% overall yield as a white solid according to Substitution reaction for synthesis of Carbamate; General Procedure I using 1-amino-3-methylurea in STEP 1; Boc Deprotection; General Procedure II in STEP 2. MS: m/z Calc'd for C₁₅H₂₂N₄O₅ [M+H]⁺339. Found, 339. ¹H NMR (400 MHz, Methanol-d₄) δ 7.26 (d, J=8.5 Hz, 2H), 6.99-6.90 (m, 2H), 5.00 (d, J=3.3 Hz, 1H), 4.46 (s, 1H), 4.20-4.14 (m, J=10.4, 7.2, 3.3 Hz, 1H), 3.80 (s, 3H), 3.63-3.49 (m, 1H), 3.23 (d, J=12.6 Hz, 1H), 3.16-3.03 (m, 1H), 2.96 (d, J=17.9 Hz, 1H), 2.77 (s, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 8.74.

### Synthetic Procedure of {2-[({[(2R,3S,4S)-4-hydroxy-2-[(4-methoxyphenyl)methyl]pyrrolidin-3-yl]oxy}carbonyl)amino]acetamido}acetic Acid (V188018)

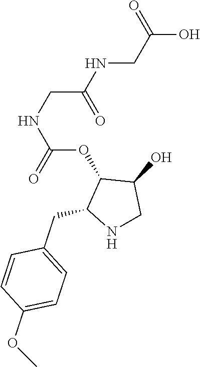 [SMILES: COc1ccc(C[C@H]2NC[C@H](O)[C@H]2OC(=O)NCC(=O)NCC(=O)O)cc1]

The title compound was prepared in 45.0% overall yield as a light orange solid according to Substitution reaction for synthesis of Carbamate; General Procedure I using tert-butyl 2-(2-aminoacetamido)acetate in STEP 1; Boc Deprotection; General Procedure II in STEP 2. MS: m/z Calc'd for C₃₁H₄₇N₃O₁₁ [M+H]⁺364. found 364. ¹H NMR (400 MHz, Methanol-d₄) δ 7.26 (dd, J=15.7, 8.2 Hz, 2H), 6.92 (d, J=8.3 Hz, 2H), 4.59 (s, 1H), 4.42 (d, J=4.1 Hz, 1H), 4.05 (s, 1H), 3.96-3.83 (m, 2H), 3.79 (s, 4H), 3.76-3.65 (m, 1H), 3.60-3.51 (m, 1H), 3.17-3.06 (m, 3H).

Prep-HPLC purification conditions: Column: Column: Welch Ultimate AQ-C18 50*250, 10 μm; Mobile Phase A: Water (10 nmol/LNH₄HCO₃), Mobile Phase B: ACN; Flow rate: 60 mL/min; Gradient: 0% B to 40% B in 20 min; Wave Length: 254 nm/220 nm; RT1(min): 41.32.

### Synthetic Procedure of (2R,3S,4S)-4-hydroxy-2-[(4-methoxyphenyl)methyl]pyrrolidin-3-yl N-[(2-oxo-1H-pyridin-4-yl)methyl]carbamate (V188053)

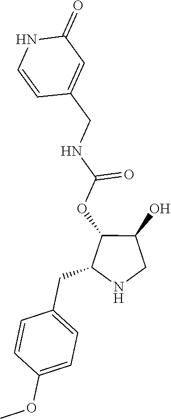 [SMILES: COc1ccc(C[C@H]2NC[C@H](O)[C@H]2OC(=O)NCc2cc[nH]c(=O)c2)cc1]

The title compound was prepared in 51.3% overall yield as a light yellow solid according to Substitution reaction for synthesis of Carbamate; General Procedure I using 4-(aminomethyl)-1H-pyridin-2-one in STEP 1; Boc Deprotection; General Procedure II in STEP 2. MS: m/z Calc'd for C₁₉H₂₃N₃O₅ [M+H]⁺374. Found, 374. ¹H NMR (400 MHz, DMSO-d₆) δ 11.46 (s, 1H), 9.46 (d, J=44.9 Hz, 1H), 9.11 (s, 1H), 7.92-7.72 (m, J=69.5, 6.3 Hz, 1H), 7.36-7.32 (m, J=7.3 Hz, 1H), 7.28-7.03 (m, 2H), 6.90 (dd, J=28.5, 8.4 Hz, 2H), 6.23 (d, J=26.1 Hz, 1H), 6.16-6.06 (m, 1H), 5.98 (s, 1H), 4.76-4.62 (m, 1H), 4.25 (s, 1H), 4.21-3.88 (m, 3H), 3.74 (d, J=4.0 Hz, 3H), 3.10 (d, J=14.5 Hz, 1H), 3.04-2.70 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 26% B in 10 min; Wave Length: 254 nm/220 nm; RT1(min): 8.42.

### Synthetic Procedure of (2R,3S,4S)-4-hydroxy-2-[(4-methoxyphenyl)methyl]pyrrolidin-3-yl N-[(2-oxo-1H-pyridin-4-yl)methyl]carbamate (V188058)

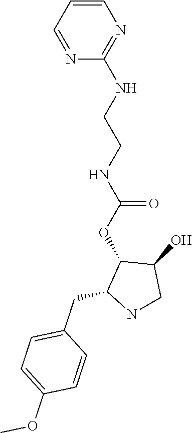

The title compound was prepared in 27.0% overall yield as a yellow oil according to Substitution reaction for synthesis of Carbamate; General Procedure I using N1-(pyrimidin-2-yl)ethane-1,2-diamine in STEP 1; Boc Deprotection; General Procedure II in STEP 2. MS: m/z Calc'd for C₁₉H₂₅N₅O₄ [M+H]⁺388. found 388. ¹H NMR (400 MHz, Methanol-d₄) δ 8.45 (s, 2H), 7.21 (d, J=8.3 Hz, 2H), 6.90 (d, J=8.2 Hz, 2H), 6.86-6.77 (m, 1H), 4.92 (d, J=3.5 Hz, 1H), 4.36 (d, J=4.2 Hz, 1H), 4.13-4.02 (m, 1H), 3.78 (s, 3H), 3.71-3.60 (m, 2H), 3.55 (dd, J=12.7, 4.3 Hz, 1H), 3.43-3.31 (m, 2H), 3.19 (d, J=12.6 Hz, 1H), 3.07 (dd, J=14.1, 7.1 Hz, 1H), 2.94 (dd, J=14.1, 8.4 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.97.

### Synthetic Procedure of (2R,3S,4S)-4-hydroxy-2-({4-[1-(1H-pyrazol-4-ylmethyl)-1,2,3-triazol-4-yl]phenyl}methyl)pyrrolidin-3-yl N-[2-(pyridin-4-yl)ethyl]carbamate (V188106)

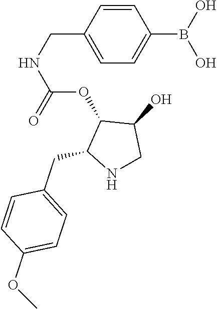 [SMILES: COc1ccc(C[C@H]2NC[C@H](O)[C@H]2OC(=O)NCc2ccc(B(O)O)cc2)cc1]

The title compound was prepared in 45.1% overall yield as a white solid according to Substitution reaction for synthesis of Carbamate; General Procedure I using 4-(aminomethyl)phenylboronic acid in STEP 1; Boc Deprotection; General Procedure II in STEP 2. MS: m/z Calc'd for C₂₀H₂₅BN₂O₆ [M+H]⁺401. found 401. ¹H NMR (400 MHz, Methanol-d₄) δ 7.78 (d, J=7.7 Hz, 1H), 7.64 (d, J=7.6 Hz, 1H), 7.34 (dd, J=13.7, 7.6 Hz, 2H), 7.23-7.16 (m, 2H), 6.87 (dd, J=7.0, 3.7 Hz, 2H), 4.42-4.28 (m, 3H), 4.16 (s, 1H), 3.59 (dd, J=12.6, 4.1 Hz, 1H), 4.32 (s, 4H), 3.23 (d, J=12.6 Hz, 1H), 3.10 (dd, J=14.1, 7.8 Hz, 1H), 2.99 (dd, J=13.4, 8.2 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 4% B to 34% B in 10 min; Wave Length: 254 nm/220 nm; RT1(min): 8.45.

### Synthetic Procedure of (2R,3S,4S)-4-hydroxy-2-[(4-methoxyphenyl)methyl]pyrrolidin-3-yl N-{[(3S)-1-methylpyrrolidin-3-yl]methyl}carbamate (V188109)

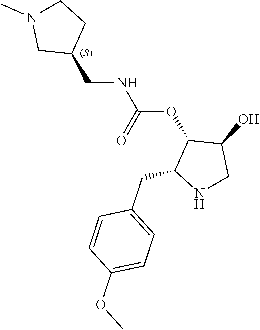

The title compound was prepared in 36.0% overall yield as a light green semi-solid according to Substitution reaction for synthesis of Carbamate; General Procedure I using 1-[(3S)-1-methylpyrrolidin-3-yl]methanamine in STEP 1; Boc Deprotection; General Procedure II in STEP 2. MS: m/z Calc'd for C₁₉H₂₉N₃O₄ [M+H]⁺364. found 364. ¹H NMR (400 MHz, Methanol-d₄) δ 7.27-7.20 (m, 2H), 6.97-6.89 (m, 2H), 4.96 (d, J=3.5 Hz, 1H), 4.39 (d, J=4.2 Hz, 1H), 4.22-4.12 (m, 1H), 3.80 (s, 4H), 3.64-3.55 (m, 1H), 3.48 (d, J=11.9 Hz, 1H), 3.31-3.23 (m, 3H), 3.21-3.08 (m, 2H), 3.05-2.98 (m, 1H), 2.96 (s, 3H), 2.89-2.78 (m, 1H), 2.68 (s, 1H), 2.28 (d, J=48.1 Hz, 1H), 1.89 (d, J=62.0 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min mL/min; Gradient: 2% B to 23% B in 10 min; Wave Length: 254 nm/220 nm; RT1(min): 7.65.

### Synthetic Procedure of (2R,3S,4S)-4-hydroxy-2-[(4-methoxyphenyl)methyl]pyrrolidin-3-yl N-[2-(1H-imidazol-2-ylamino)ethyl]carbamate (V187825)

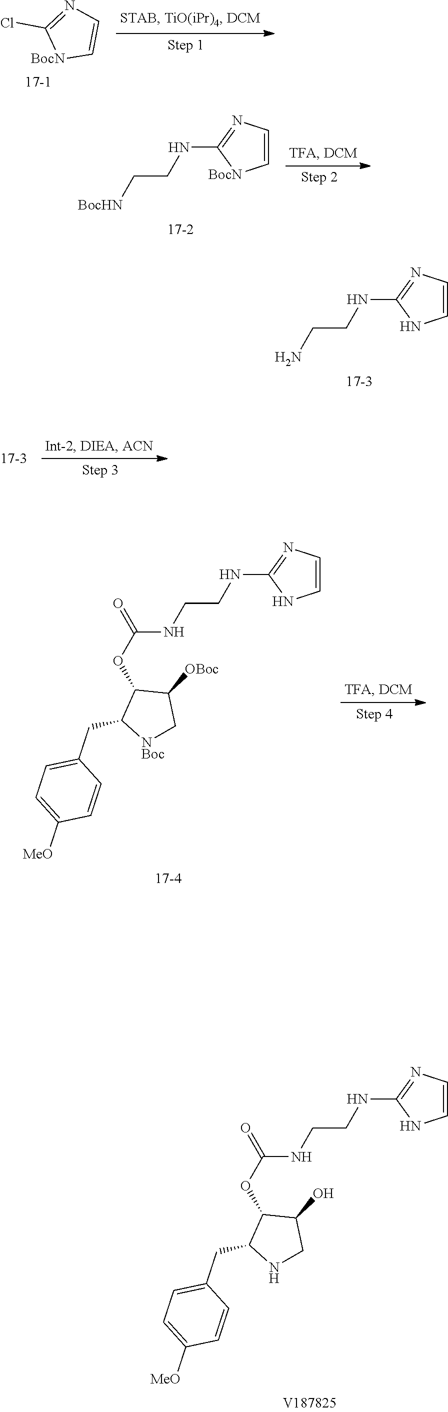

Step 1: To a stirred solution of tert-butyl 2-aminoimidazole-1-carboxylate (600 mg, 3.28 mmol, 1.0 eq.) and t tert-butyl 2-chloro-1H-imidazole-1-carboxylate (521.3 mg, 3.28 mmol, 1.0 eq.) in DMF (5 mL) was added tetrakis(propan-2-yloxy)titanium (1861.6 mg, 6.55 mmol, 2.0 eq.). The mixture was stirred at 25° C. for overnight. NaBH(OAc)₃ (1388.2 mg, 6.55 mmol, 2.0 eq.) was added at 0° C. The resulting mixture was stirred at 25° C. for another 1 h. Desired product could be detected by LCMS. The reaction was quenched with Water at 0° C. The aqueous layer was extracted with CH₂Cl₂ (3×10 mL). The residue was purified by reversed-phase flash chromatography to afford tert-butyl 2-({2-[(tert-butoxycarbonyl)amino]ethyl}amino)imidazole-1-carboxylate (150 mg, 14.0%) as a white solid. MS: m/z: Calc'd for C₁₅H₂₆N₄O₄ [M+H]⁺327. Found, 327.

Step 2: To a stirred solution of tert-butyl 2-({2-[(tert-butoxycarbonyl)amino]ethyl}amino)imidazole-1-carboxylate (140 mg, 0.43 mmol, 1.0 eq.) in DCM (4 mL) was added TFA (2 mL). The resulting mixture was stirred at 25° C. for 1 h. Desired product could be detected by LCMS. The resulting mixture was concentrated under reduced pressure. The resulting mixture was used in the next step directly without further purification. MS: m/z: Calc'd for C₅H₁₀N₄ [M+H]⁺127. Found, 127.

### Step 3 and 4

The title compound was prepared in 9.6% overall yield as a light yellow semi-solid according to Substitution reaction for synthesis of Carbamate; General Procedure I using N1-(1H-imidazol-2-yl)ethane-1,2-diamine (17-3) in STEP 3; Boc Deprotection; General Procedure II in STEP 4. MS: m/z Calc'd for C₁₈H₂₅N₅O₄ [M+H]⁺376. Found, 376. ¹H NMR (400 MHz, DMSO-d₆) δ 7.22 (d, J=8.1 Hz, 2H), 6.94-6.82 (m, 3H), 4.95 (s, 2H), 4.37 (d, J=4.1 Hz, 1H), 4.17-4.13 (m, J=7.9, 3.4 Hz, 1H), 3.79 (s, 3H), 3.57 (dd, J=12.7, 4.3 Hz, 1H), 3.50-3.40 (m, J=11.4, 7.6, 6.2 Hz, 4H), 3.20 (d, J=12.6 Hz, 1H), 3.09 (dd, J=14.1, 7.0 Hz, 1H), 2.96 (dd, J=14.2, 8.6 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.52.

### Synthetic Procedure of (2R,3S,4S)-4-hydroxy-2-[(4-methoxyphenyl)methyl]pyrrolidin-3-yl N-{2-[(3S)-pyrrolidin-3-yl]ethyl}carbamate (V187878)

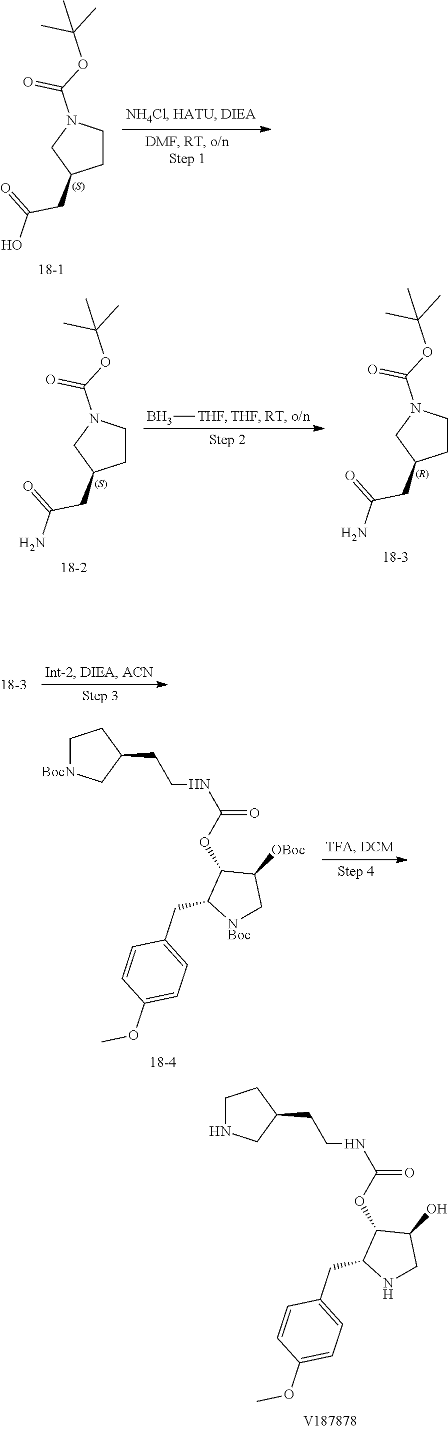

Step 1: To a stirred solution of [(3S)-1-(tert-butoxycarbonyl)pyrrolidin-3-yl]acetic acid (500 mg, 2.18 mmol, 1.0 eq.) and NH₄Cl (583.3 mg, 10.91 mmol, 5.0 eq.) in DMF (10 mL) was added HATU (1658.4 mg, 4.36 mmol, 2.0 eq.) and DIEA (1127.4 mg, 8.72 mmol, 4.0 eq.). The resulting mixture was stirred over night at room temperature. Upon completion, the resulting mixture was extracted with EtOAc (3×10 mL). The combined organic layers were washed with water (3×10 mL), dried over anhydrous Na₂SO₄. After filtration, the filtrate was concentrated under reduced pressure. The residue was purified by reversed-phase flash chromatography to afford tert-butyl (3S)-3-(carbamoylmethyl)pyrrolidine-1-carboxylate (400 mg, 80.3%) as a light yellow oil. MS: m/z: Calc'd for C₁₁H₂₀N₂O₃ [M+H+22]⁺251. Found, 251.

Step 2: To a stirred solution of tert-butyl (3S)-3-(carbamoylmethyl)pyrrolidine-1-carboxylate (150 mg, 0.66 mmol, 1.0 eq.) in THF (5 mL) was added BH₃-THF (3.29 mL, 3.285 mmol, 5.0 eq.) in portions at −78° C. The resulting mixture was stirred at −78° C. for 1 h. Desired product could be detected by LCMS. The reaction was quenched with MeOH at 0° C. The resulting mixture was concentrated under reduced pressure. The resulting mixture was used in the next step directly without further purification. MS: m/z: Calc'd for C₁₁H₂₂N₂O₂ [M+H]⁺215. Found, 215.

### Step 3 and 4

The title compound was prepared in 35.9% overall yield as a yellow semi-solid according to Substitution reaction for synthesis of Carbamate; General Procedure I using tert-butyl (3R)-3-(2-aminoethyl)pyrrolidine-1-carboxylate (18-3) in STEP 3; Boc Deprotection; General Procedure II in STEP 4. MS: m/z Calc'd for C₁₉H₂₉N₃O₄ [M+H]⁺364. Found, 364. ¹H NMR (400 MHz, Methanol-d₄) δ 7.28-7.21 (m, 2H), 6.96-6.90 (m, 2H), 4.95 (d, J=3.5 Hz, 1H), 4.38 (d, J=4.1 Hz, 1H), 4.19-4.15 (m, 1H), 3.80 (s, 3H), 3.57 (dd, J=12.6, 4.3 Hz, 1H), 3.50 (dd, J=11.5, 7.6 Hz, 1H), 3.44-3.38 (m, 1H), 3.27 (d, J=8.6 Hz, 1H), 3.26-3.17 (m, 3H), 3.11 (dd, J=14.1, 7.1 Hz, 1H), 2.98 (dd, J=14.1, 8.5 Hz, 1H), 2.88 (dd, J=11.6, 9.1 Hz, 1H), 2.44-2.21 (m, 2H), 1.81-1.61 (m, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 8.77.

### Synthetic Procedure of (2R,3S,4S)-4-hydroxy-2-[(4-methoxyphenyl)methyl]pyrrolidin-3-yl N-{[1,1′-biphenyl]-4-ylmethyl}carbamate (V187977)

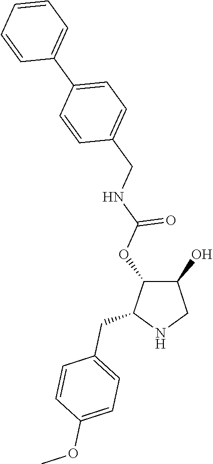 [SMILES: COc1ccc(C[C@H]2NC[C@H](O)[C@H]2OC(=O)NCc2ccc(-c3ccccc3)cc2)cc1]

The title compound was prepared in 33.4% overall yield as a white solid according to Substitution reaction for synthesis of Carbamate; General Procedure I using 1-{[1,1′-biphenyl]-4-yl}methanamine in STEP 1; Boc Deprotection; General Procedure II in STEP 2. MS: m/z Calc'd for C₂₆H₂₈N₂O₄[M+H]⁺ 433, Found 433. ¹H NMR (400 MHz, Methanol-d₄) δ 7.64-7.54 (m, 4H), 7.44 (t, J=7.7 Hz, 5H), 7.22-7.15 (m, 2H), 6.89-6.81 (m, 2H), 4.96-4.91 (m, 1H), 4.42-4.30 (d, J=15.4 Hz, 3H), 4.16 (td, J=7.8, 3.5 Hz, 1H), 3.74 (s, 3H), 3.59 (dd, J=12.6, 4.3 Hz, 1H), 3.23 (d, J=12.6 Hz, 1H), 3.11 (dd, J=14.0, 7.8 Hz, 1H), 2.98 (dd, J=14.0, 8.1 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 18% B to 48% B in 10 min; Wave Length: 254 nm/220 nm; RT1(min): 8.3.

### Synthetic Procedure of (2R,3S,4S)-4-hydroxy-2-[(4-methoxyphenyl)methyl]pyrrolidin-3-yl N-(carbamoylmethyl)carbamate (V188002)

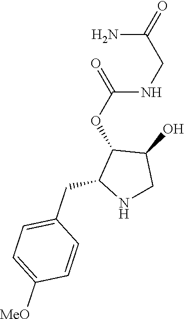 [SMILES: COc1ccc(C[C@H]2NC[C@H](O)[C@H]2OC(=O)NCC(N)=O)cc1]

The title compound was prepared in 12.0% overall yield as a light yellow semi-solid according to Substitution reaction for synthesis of Carbamate; General Procedure I using glycinamide in STEP 1; Boc Deprotection; General Procedure II in STEP 2. MS: m/z Calc'd for C₁₅H₂₁N₃O₅ [M+H]⁺324. Found, 324. ¹H NMR (400 MHz, Methanol-d₄) δ 7.30-7.22 (m, 2H), 6.98-6.90 (m, 2H), 4.96 (d, J=2.9 Hz, 1H), 4.44 (d, J=4.4 Hz, 1H), 4.20-4.11 (m, 1H), 3.85 (d, J=2.9 Hz, 2H), 3.80 (s, 3H), 3.62 (dd, J=12.6, 4.4 Hz, 1H), 3.22 (d, J=12.6 Hz, 1H), 3.15 (dd, J=14.2, 7.1 Hz, 1H), 2.99 (dd, J=14.2, 8.6 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 8.25.

### Synthetic Procedure of 1-{[(2R,3S,4S)-4-hydroxy-2-[(4-methoxyphenyl)methyl]pyrrolidin-3-yl]oxy}-N-[(methylcarbamoyl)amino]formamide (V188003)

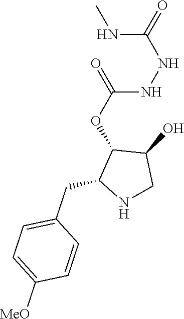 [SMILES: CNC(=O)NNC(=O)O[C@@H]1[C@@H](O)CN[C@@H]1Cc1ccc(OC)cc1]

The title compound was prepared in 42.8% overall yield as a white solid according to Substitution reaction for synthesis of Carbamate; General Procedure I using 1-amino-3-methylurea in STEP 1; Boc Deprotection; General Procedure II in STEP 2. MS: m/z Calc'd for C₁₅H₂₂N₄O₅ [M+H]⁺339. Found, 339. ¹H NMR (400 MHz, Methanol-d₄) δ 7.26 (d, J=8.5 Hz, 2H), 6.99-6.90 (m, 2H), 5.00 (d, J=3.3 Hz, 1H), 4.46 (s, 1H), 4.20-4.14 (m, 1H), 3.80 (s, 3H), 3.63-3.49 (m, 1H), 3.23 (d, J=12.6 Hz, 1H), 3.16-3.03 (m, 1H), 2.96 (d, J=17.9 Hz, 1H), 2.77 (s, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 8.74.

### Synthetic Procedure of (2R,3S,4S)-4-hydroxy-2-[(4-methoxyphenyl)methyl]pyrrolidin-3-yl N-{2-[(2S)-pyrrolidin-2-ylformamido]ethyl}carbamate (V188015)

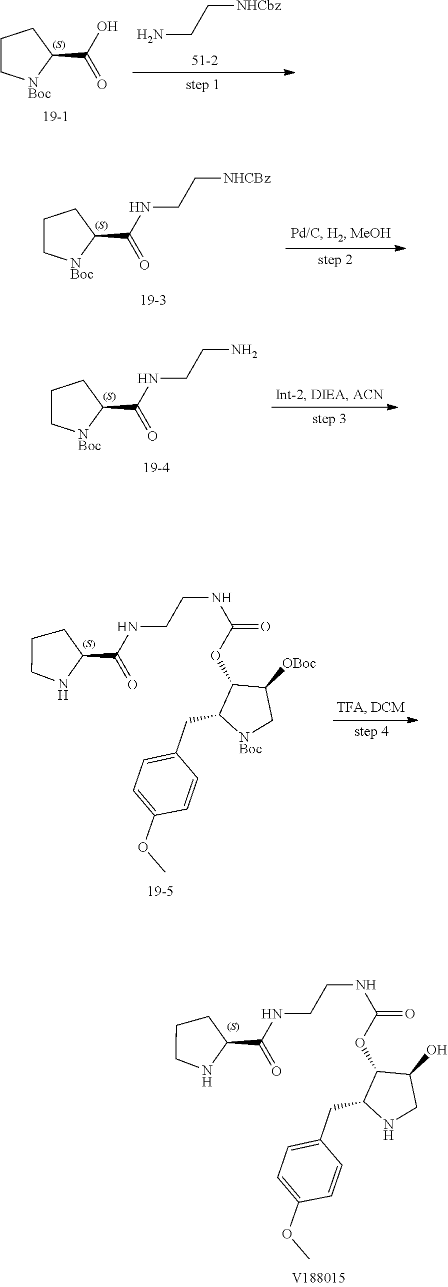

Step 1: To a stirred solution of (2S)-1-(tert-butoxycarbonyl)pyrrolidine-2-carboxylic acid (500 mg, 2.32 mmol, 1 eq.) and benzyl N-(2-aminoethyl)carbamate (902.3 mg, 4.64 mmol, 2 eq.) in DCM (5 mL) were added DCC (958.8 mg, 4.64 mmol, 2 eq.) and DMAP (283.7 mg, 2.32 mmol, 1 eq.) at 0° C. The resulting mixture was stirred at room temperature for overnight. The resulting mixture was concentrated under reduced pressure. The crude product was purified by reversed phase column chromatography to afford tert-butyl (2S)-2-[(2-{[(benzyloxy)carbonyl]amino}ethyl)carbamoyl]pyrrolidine-1-carboxylate (625 mg, 3436.5%) as a light yellow solid. MS: m/z: Calc'd for C₂₀H₂₉N₃O₅[M+H]⁺392. Found, 392.

Step 2: To a stirred solution of tert-butyl (2S)-2-[(2-{[(benzyloxy)carbonyl]amino}ethyl)carbamoyl]pyrrolidine-1-carboxylate (300 mg, 0.76 mmol, 1 eq.) in MeOH (6 mL) was added Pd/C (150.0 mg) at 0° C. The resulting mixture was stirred at room temperature for 2 h under hydrogen atmosphere. Upon completion, filtered, concentrated to afford tert-butyl (2S)-2-[(2-aminoethyl)carbamoyl]pyrrolidine-1-carboxylate (180 mg, 91.2%) as a yellow oil which was used directly in the next step without further purification. MS: m/z: Calc'd for C₁₂H₂₃N₃O₃ [M+H]⁺258. Found, 258.

Step 3 and 4: The title compound was prepared in 35.5% overall yield as a white solid according to Substitution reaction for synthesis of Carbamate; General Procedure I using tert-butyl (2S)-2-[(2-aminoethyl)carbamoyl]pyrrolidine-1-carboxylate (19-4) in STEP 3; Boc Deprotection; General Procedure II in STEP 4. MS: m/z Calc'd for C₂₀H₃₀N₄O₅[M+H]⁺407. Found, 407. ¹H NMR (400 MHz, Methanol-d₄) δ 7.29-7.20 (m, 2H), 6.98-6.90 (m, 2H), 4.96 (d, J=3.5 Hz, 1H), 4.40 (d, J=4.1 Hz, 1H), 4.24-4.17 (dd, J=8.7, 6.9, 3.5 Hz, 2H), 3.80 (s, 3H), 3.57 (dd, J=12.6, 4.3 Hz, 1H), 3.51-3.22 (d, J=12.6 Hz, 5H), 3.20-3.16 (m, 1H), 3.11 (dd, J=14.1, 6.9 Hz, 1H), 3.07-2.99 (m, 1H), 2.97 (dd, J=14.2, 8.7 Hz, 1H), 2.41-2.39 (m, 1H), 2.06-2.03 (m, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 9.

### Synthetic Procedure of (2R,3S,4S)-4-hydroxy-2-({4-[1-(1H-pyrazol-4-ylmethyl)-1,2,3-triazol-4-yl]phenyl}methyl)pyrrolidin-3-yl Acetate (V188016)

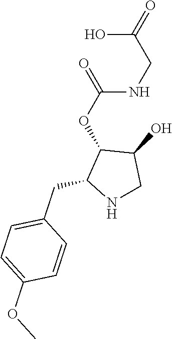 [SMILES: COc1ccc(C[C@H]2NC[C@H](O)[C@H]2OC(=O)NCC(=O)O)cc1]

The title compound was prepared in 17.7% overall yield as a yellow semi-solid according to Substitution reaction for synthesis of Carbamate; General Procedure I using glycine in STEP 1; Boc Deprotection; General Procedure II in STEP 2. MS: m/z Calc'd for C₁₅H₂₀N₂O₆[M+H]⁺325, Found 325. ¹H NMR (400 MHz, Methanol-d₄) δ 7.30-7.22 (m, 2H), 6.98-6.89 (m, 2H), 4.96 (d, J=3.5 Hz, 1H), 4.42 (d, J=4.3 Hz, 1H), 4.15-4.12 (m, 1H), 4.00-3.87 (m, 2H), 3.80 (s, 3H), 3.65-3.50 (m, 1H), 3.23 (d, J=12.6 Hz, 1H), 3.15 (dd, J=14.1, 7.3 Hz, 1H), 2.99 (dd, J=14.2, 8.4 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.67.

### Synthetic Procedure of (2R,3S,4S)-4-hydroxy-2-[(4-methoxyphenyl)methyl]pyrrolidin-3-yl N-[(methylcarbamoyl)methyl]carbamate (V188017)

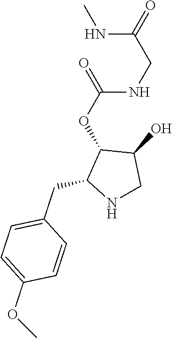 [SMILES: CNC(=O)CNC(=O)O[C@@H]1[C@@H](O)CN[C@@H]1Cc1ccc(OC)cc1]

The title compound was prepared in 32.6% overall yield as a yellow oil according to Substitution reaction for synthesis of Carbamate; General Procedure I using 2-amino-N-methylacetamide in STEP 1; Boc Deprotection; General Procedure II in STEP 2. MS: m/z Calc'd for C₁₆H₂₃N₃O₅[M+H]⁺ 338, Found 338. ¹H NMR (400 MHz, Methanol-d₄) δ 7.26 (d, J=8.2 Hz, 2H), 6.94 (d, J=8.3 Hz, 2H), 4.97 (d, J=3.4 Hz, 1H), 4.44 (d, J=4.2 Hz, 1H), 4.21-4.11 (m, 1H), 3.88-3.74 (m, 5H), 3.62 (dd, J=12.6, 4.4 Hz, 1H), 3.26-3.13 (m, 1H), 3.10-3.03 (m, 1H), 2.99 (dd, J=14.2, 8.6 Hz, 1H), 2.79 (s, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.73.

### Synthetic Procedure of (2R,3S,4S)-4-hydroxy-2-[(4-methoxyphenyl)methyl]pyrrolidin-3-yl N-[(carbamoylmethylcarbamoyl)methyl]carbamate (V188021)

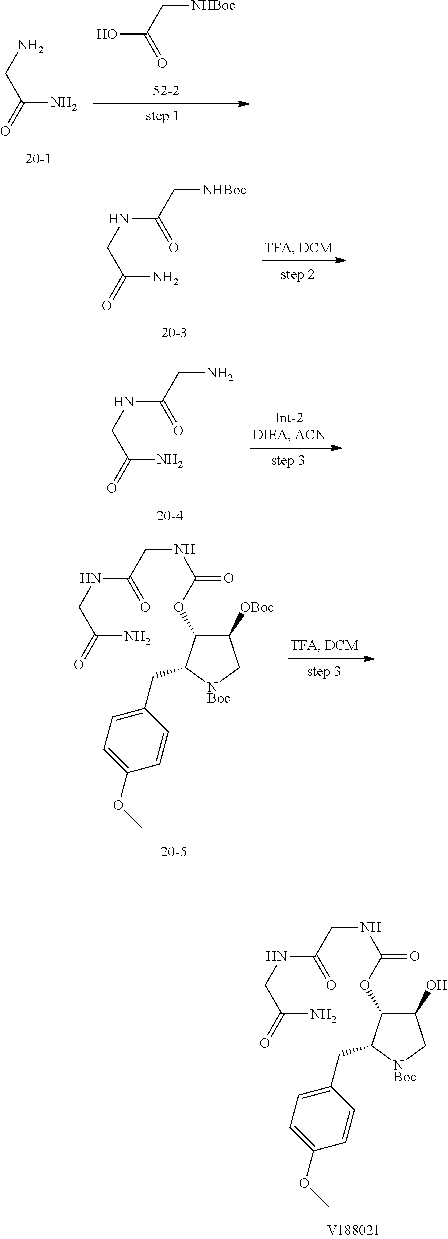

Step 1: To a stirred solution of glycinamide (500 mg, 6.74 mmol, 1 eq.) and [(tert-butoxycarbonyl)amino]acetic acid (2364.7 mg, 13.49 mmol, 2 eq.) in DCM (5 mL) were added ethyl 2-ethoxy-1,2-dihydroquinoline-1-carboxylate (5007.0 mg, 20.24 mmol, 3 eq.) at 0° C. The resulting mixture was stirred at room temperature for overnight. The crude product was purified by reversed phase flash chromatography to afford tert-butyl N-[(carbamoylmethylcarbamoyl)methyl]carbamate (200 mg, 12.8%) as a light yellow oil. MS: m/z: Calc'd for C₉H₇N₃O₄[M+H−56]⁺176. Found, 176.

Step 2: To a stirred solution of tert-butyl N-[(carbamoylmethylcarbamoyl)methyl]carbamate (195 mg, 0.84 mmol, 1 eq.) in DCM (3 mL) were added TFA (0.6 mL) at 0° C. The resulting mixture was stirred at room temperature for 2 h. The resulting mixture was concentrated under reduced pressure to afford 2-amino-N-(carbamoylmethyl)acetamide (180 mg, 162.7%) as a light yellow oil. MS: m/z: Calc'd for C₄H₉N₃O₂[M+H+22]⁺154. Found, 154.

### Step 3 and 4

The title compound was prepared in 20.3% overall yield as a white solid according to Substitution reaction for synthesis of Carbamate; General Procedure I using 2-amino-N-(carbamoylmethyl)acetamide (20-4) in STEP 3; Boc Deprotection; General Procedure II in STEP 4. MS: m/z Calc'd for C₁₇H₂₄N₄O₆[M+H]⁺381. Found, 381. ¹H NMR (400 MHz, Methanol-d₄) δ 7.30-7.23 (m, 2H), 6.98-6.90 (m, 2H), 4.98-4.96 (m, 1H), 4.45 (dd, J=9.1, 3.8 Hz, 1H), 4.16-4.13 (m, 1H), 4.04-3.83 (m, 4H), 3.80 (s, 3H), 3.59-3.57 (m, 1H), 3.26-3.15 (m, 1H), 3.18-3.08 (m, 1H), 3.00-2.98 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 9.33.

### Synthetic Procedure of (2R,3S,4S)-4-hydroxy-2-[(4-methoxyphenyl)methyl]pyrrolidin-3-yl N-[(2-methoxypyridin-4-yl)methyl]carbamate (V188051)

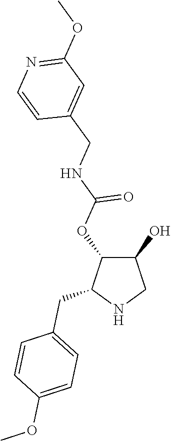 [SMILES: COc1ccc(C[C@H]2NC[C@H](O)[C@H]2OC(=O)NCc2ccnc(OC)c2)cc1]

The title compound was prepared in 36.2% overall yield as a light yellow semi-solid according to Substitution reaction for synthesis of Carbamate; General Procedure I using 1-(2-methoxypyridin-4-yl)methanamine in STEP 1; Boc Deprotection; General Procedure II in STEP 2. MS: m/z Calc'd for C₂₀H₂₅N₃O₅[M+H]⁺ 388, Found 388. ¹H NMR (400 MHz, Methanol-d₄) δ 8.13 (d, J=5.4 Hz, 1H), 7.23 (d, J=8.3 Hz, 2H), 7.08-6.96 (m, 1H), 6.91-6.86 (m, 3H), 4.93 (s, 1H), 4.41 (dd, J=10.3, 6.1 Hz, 2H), 4.32 (d, J=16.3 Hz, 1H), 4.19-4.17 (m, 1H), 3.96 (s, 3H), 3.80 (s, 3H), 3.61 (dd, J=12.5, 4.4 Hz, 1H), 3.25 (d, J=12.6 Hz, 1H), 3.13 (dd, J=14.0, 7.8 Hz, 1H), 3.01 (dd, J=14.0, 8.1 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 31% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-[(3-chloropyridin-4-yl)methyl]carbamate (V188052)

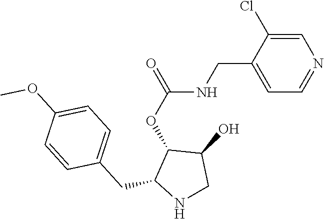 [SMILES: COc1ccc(C[C@H]2NC[C@H](O)[C@H]2OC(=O)NCc2ccncc2Cl)cc1]

The title compound was prepared in 25.3% overall yield as a white solid according to Substitution reaction for synthesis of Carbamate; General Procedure I using 1-(3-chloropyridin-4-yl)methanamine in STEP 1; Boc Deprotection; General Procedure II in STEP 2. MS: m/z Calc'd for C₁₉H₂₂ClN₃O₄[M+H]⁺392, Found 392. ¹H NMR (400 MHz, Methanol-d₄) δ 8.59 (s, 1H), 8.50 (d, J=5.1 Hz, 1H), 7.46 (d, J=5.1 Hz, 1H), 7.28-7.20 (m, 2H), 6.95-6.87 (m, 2H), 5.00-4.95 (m, 1H), 4.59-4.40 (m, 3H), 4.21-4.19 (m, 1H), 3.80 (s, 3H), 3.61 (dd, J=12.6, 4.3 Hz, 1H), 3.25 (d, J=12.6 Hz, 1H), 3.14 (dd, J=14.1, 7.4 Hz, 1H), 3.01 (dd, J=14.1, 8.4 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 32% B in 10 min; Wave Length: 254 nm/220 nm; RT1(min): 9.63.

### Synthetic Procedure of (2R,3S,4S)-4-hydroxy-2-[(4-methoxyphenyl)methyl]pyrrolidin-3-yl N-[(2-oxo-1H-pyridin-4-yl)methyl]carbamate (V188053)

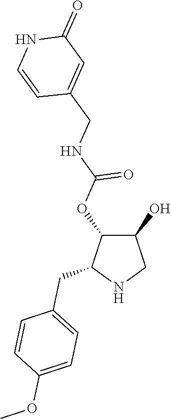 [SMILES: COc1ccc(C[C@H]2NC[C@H](O)[C@H]2OC(=O)NCc2cc[nH]c(=O)c2)cc1]

The title compound was prepared in 51.3% overall yield as a light yellow solid according to Substitution reaction for synthesis of Carbamate; General Procedure I using 4-(aminomethyl)-1H-pyridin-2-one in STEP 1; Boc Deprotection; General Procedure II in STEP 2. MS: m/z Calc'd for C₁₉H₂₃N₃O₅ [M+H]⁺374. Found, 374. ¹H NMR (400 MHz, DMSO-d₆) δ 11.46 (s, 1H), 9.46 (d, J=44.9 Hz, 1H), 9.11 (s, 1H), 7.92-7.72 (m, 1H), 7.36-7.32 (m, 1H), 7.28-7.03 (m, 2H), 6.90 (dd, J=28.5, 8.4 Hz, 2H), 6.23 (d, J=26.1 Hz, 1H), 6.16-6.06 (m, 1H), 5.98 (s, 1H), 4.76-4.62 (m, 1H), 4.25 (s, 1H), 4.21-3.88 (m, 3H), 3.74 (d, J=4.0 Hz, 3H), 3.10 (d, J=14.5 Hz, 1H), 3.04-2.70 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 26% B in 10 min; Wave Length: 254 nm/220 nm; RT1(min): 8.42.

### Synthetic Procedure of (2R,3S,4S)-4-hydroxy-2-[(4-methoxyphenyl)methyl]pyrrolidin-3-yl N-[2-(pyridin-2-ylamino)ethyl]carbamate (V188059)

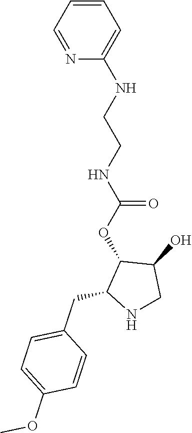 [SMILES: COc1ccc(C[C@H]2NC[C@H](O)[C@H]2OC(=O)NCCNc2ccccn2)cc1]

The title compound was prepared in 39.2% overall yield as a yellow oil according to Substitution reaction for synthesis of Carbamate; General Procedure I using N1-(pyridin-2-yl)ethane-1,2-diamine in STEP 1; Boc Deprotection; General Procedure II in STEP 2. MS: m/z Calc'd for C₂₀H₂₆N₄O₄ [M+H]⁺387. found 387. ¹H NMR (400 MHz, Methanol-d₄) δ 7.92-7.81 (m, 1H), 7.87 (dd, J=6.5, 1.7 Hz, 1H), 7.24-7.17 (m, 2H), 7.12 (d, J=9.1 Hz, 1H), 6.94-6.86 (m, 3H), 4.94 (d, J=3.4 Hz, 1H), 4.35 (d, J=4.3 Hz, 1H), 4.13-4.01 (m, 1H), 3.78 (s, 3H), 3.57-3.42 (m, 3H), 3.47-3.35 (m, 2H), 3.20 (d, J=12.6 Hz, 1H), 3.06 (dd, J=14.1, 7.0 Hz, 1H), 2.95 (dd, J=14.2, 8.5 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 24% B in 10 min; Wave Length: 254 nm/220 nm; RT1(min): 8.52.

### Synthetic Procedure of (2R,3S,4S)-4-hydroxy-2-[(4-methoxyphenyl)methyl]pyrrolidin-3-yl N-[2-(7H-purin-6-ylamino)ethyl]carbamate (V188060)

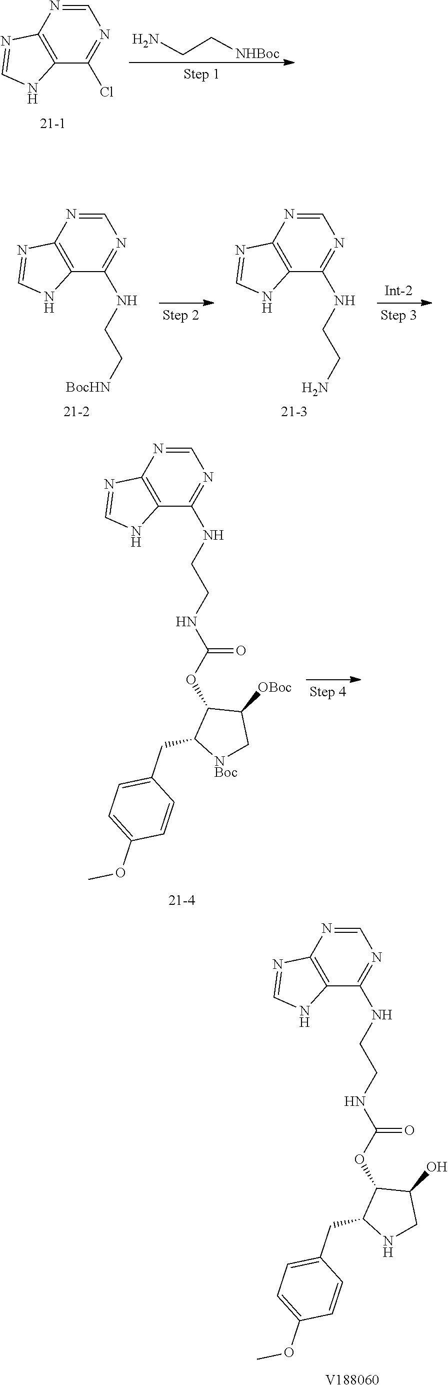

Step 1: To a stirred mixture of 6-chloropurine (21-1, 500 mg, 3.23 mmol, 1 eq.) and tert-butyl N-(2-aminoethyl)carbamate (777.4 mg, 4.85 mmol, 1.5 eq.) in n-BuOH (10 mL) was added DIPEA (836.2 mg, 6.47 mmol, 2 eq.) at room temperature under nitrogen atmosphere. The resulting mixture was stirred at 110° C. for overnight. The precipitated solids were collected by filtration and washed with EtOH (2×6 mL). The resulting solid was dried under vacuum. This resulted in tert-butyl N-[2-(7H-purin-6-ylamino)ethyl]carbamate (700 mg, 77.7%) as a yellow solid. MS: m/z: Calc'd for C₁₂H₁₈N₆O₂ [M+H]⁺279. found 279.

Step 2: To a stirred mixture of tert-butyl N-[2-(7H-purin-6-ylamino)ethyl]carbamate (21-2, 200 mg, 0.72 mmol) in DCM (5 mL) was added TFA (1 mL) at room temperature. The resulting mixture was stirred at room temperature for 3h. Upon completion, concentrated under vacuum to afford N1-(7H-purin-6-yl)ethane-1,2-diamine (120 mg, 93.7%) as a yellow oil. MS: m/z: Calc'd for C₇H₁₀N₆ [M+H]⁺179. found 179.

### Step 3 and 4

The title compound was prepared in 28.6% overall yield as a yellow solid according to Substitution reaction for synthesis of Carbamate; General Procedure I using N1-(7H-purin-6-yl)ethane-1,2-diamine (21-3) in STEP 3; Boc Deprotection; General Procedure II in STEP 4. MS: m/z Calc'd for C₂₀H₂₅N₇O₄ [M+H]⁺428. found 428. ¹H NMR (400 MHz, Methanol-d₄) δ 8.44 (s, 1H), 8.30-8.24 (m, 1H), 7.19-7.11 (m, 2H), 6.92-6.83 (m, 2H), 4.92 (d, J=3.4 Hz, 1H), 4.35-4.29 (m, 1H), 4.10-4.01 (m, 1H), 3.88 (s, 2H), 3.77 (s, 3H), 3.57-3.52 (m, 2H), 3.52-3.46 (m, 1H), 3.17 (d, J=12.6 Hz, 1H), 3.01 (dd, J=14.2, 6.9 Hz, 1H), 2.86-2.74 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.42.

### Synthetic Procedure of (2R,3S,4S)-4-hydroxy-2-[(4-methoxyphenyl)methyl]pyrrolidin-3-yl N-{2-[(3R)-pyrrolidin-3-ylamino]ethyl}carbamate (V188073)

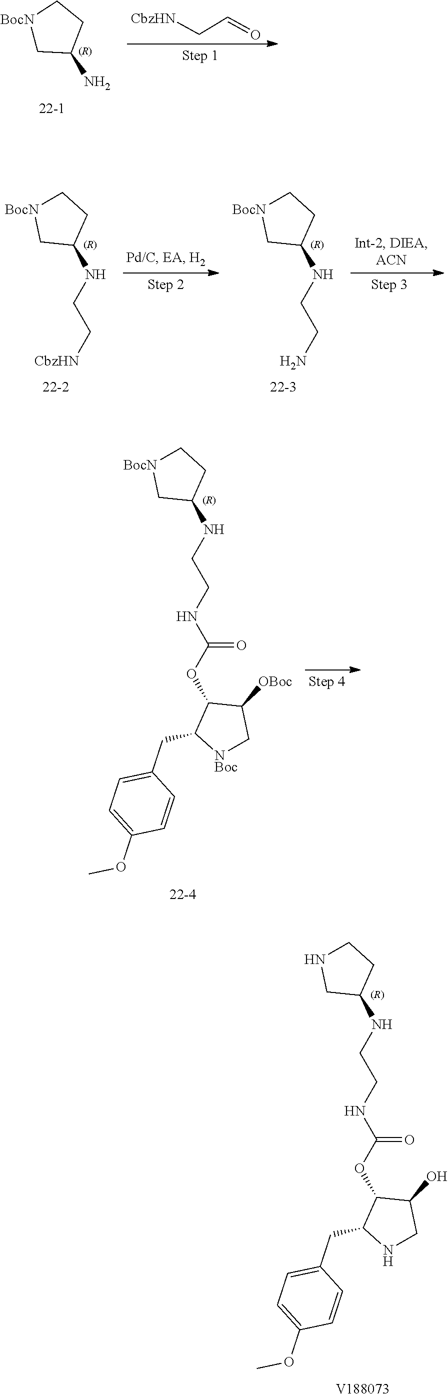

Step 1: A solution of tert-butyl (3R)-3-aminopyrrolidine-1-carboxylate (22-1, 100 mg, 0.53 mmol, 1 eq.) and DIEA (346.9 mg, 2.68 mmol, 5 eq.) in DCM (5 mL) was treated with HOAc (225.6 mg, 3.75 mmol, 7 eq.) and benzyl N-(2-oxoethyl)carbamate (103.7 mg, 0.53 mmol, 1 eq.) for 0.5 h at room temperature followed by the addition of NaBH(OAc)₃ (227.5 mg, 1.07 mmol, 2 eq.) at room temperature. The resulting mixture was stirred for overnight at room temperature. The reaction was quenched by the addition of Water (1 mL) at 0° C. The resulting mixture was concentrated under reduced pressure. The residue was purified by reversed-phase flash chromatography to afford tert-butyl (3R)-3-[(2-{[(benzyloxy)carbonyl]amino}ethyl)amino]pyrrolidine-1-carboxylate (220 mg) as a yellow oil. MS: m/z: Calc'd for C₁₉H₂₉N₃O₄ [M+H]⁺364. found 364.

Step 2: To a stirred mixture of tert-butyl (3R)-3-[(2-{[(benzyloxy)carbonyl]amino}ethyl)amino]pyrrolidine-1-carboxylate (22-2, 210 mg, 0.57 mmol, 1 equiv) in EA (7 mL) was added Pd/C (122.9 mg, 1.15 mmol, 2 equiv) at room temperature. The reaction was placed under vacuum, sonicated and backfilled with hydrogen. The resulting mixture was stirred for overnight. The resulting mixture was filtered, the filter cake was washed with EA (7 mL) (2×2 mL). The filtrate was concentrated under reduced pressure. This resulted in tert-butyl (3R)-3-[(2-aminoethyl)amino]pyrrolidine-1-carboxylate (150 mg) as a yellow oil. MS: m/z: Calc'd for C₁₁H₂₃N₃O₂ [M+H]⁺ 230. found 230.

### Step 3 and 4

The title compound was prepared in 24.7% overall yield as a yellow oil according to Substitution reaction for synthesis of Carbamate; General Procedure I using tert-butyl (3R)-3-[(2-aminoethyl)amino]pyrrolidine-1-carboxylate (22-3) in STEP 3; Boc Deprotection; General Procedure II in STEP 4. MS: m/z Calc'd for C₁₉H₃₀N₄O₄[M+H]⁺379. found 379. ¹H NMR (400 MHz, Methanol-d₄) δ 7.29-7.20 (m, 2H), 6.98-6.90 (m, 2H), 5.02 (d, J=3.5 Hz, 1H), 4.43 (d, J=4.6 Hz, 1H), 4.18-4.05 (m, 1H), 4.09-3.98 (m, 1H), 3.80 (s, 3H), 3.72 (dd, J=13.0, 7.7 Hz, 1H), 3.65-3.37 (m, 5H), 3.34 (s, 1H), 3.27-3.17 (m, 3H), 3.15-3.07 (m, 1H), 2.98 (dd, J=14.3, 9.1 Hz, 1H), 2.54-2.41 (m, 1H), 2.22-2.11 (m, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 15% B in 15 min; Wave Length: 254 nm/220 nm; RT1(min): 18.48.

### Synthetic Procedure of: (2R,3S,4S)-4-hydroxy-2-[(4-methoxyphenyl)methyl]pyrrolidin-3-yl N-[(4,5-dichloro-1H-imidazol-2-yl)methyl]carbamate (V188084)

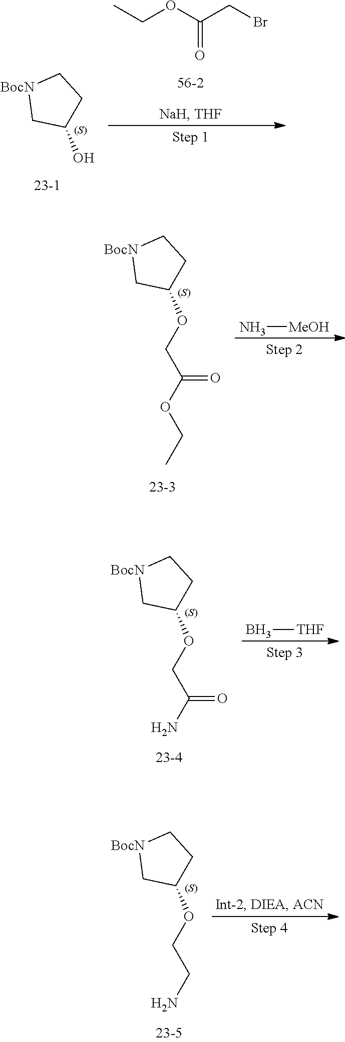

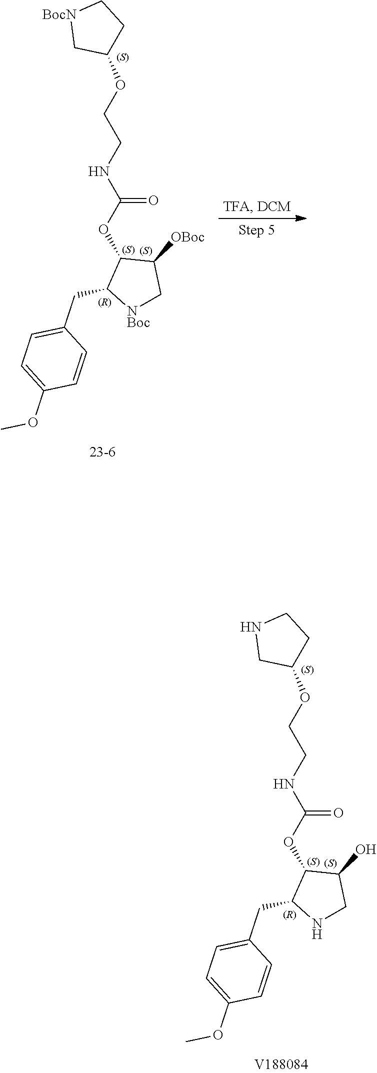

Step 1: To a stirred solution of tert-butyl (3S)-3-hydroxypyrrolidine-1-carboxylate (23-1, 1 g, 5.341 mmol, 1 eq.) in THF (10 mL) was added NaH (0.51 g, 21.36 mmol, 4.0 eq.) in portions at 0° C. The mixture was stirred at 0° C. for 30 min. Ethyl bromoacetate (1.8 g, 10.68 mmol, 2.0 eq.) was added in portions at 0° C. The resulting mixture was stirred at 25° C. for overnight. Upon completion, concentrated, the residue was purified by reversed-phase flash chromatography to afford tert-butyl (3S)-3-(2-ethoxy-2-oxoethoxy)pyrrolidine-1-carboxylate (800 mg, 54.8%) as a white solid. MS: m/z: Calc'd for C₁₃H₂₃NO₅ [M+H]⁺218. Found, 218.

Step 2: To a stirred solution of tert-butyl (3S)-3-(2-ethoxy-2-oxoethoxy)pyrrolidine-1-carboxylate (23-3, 500 mg, 1.83 mmol, 1.0 eq.) in NH₃(g) in MeOH (15 mL) was stirred at room temperature for 2 h. Desired product could be detected by LCMS. The resulting mixture was concentrated under reduced pressure. The residue was purified by reversed-phase flash chromatography to afford tert-butyl (3S)-3-(carbamoylmethoxy)pyrrolidine-1-carboxylate (400 mg, 89.5%) as a white solid. MS: m/z: Calc'd for C₁₁H₂₀N₂O₄ [M+H−56]⁺189. Found, 189.

Step 3: To a stirred solution of tert-butyl (3S)-3-(carbamoylmethoxy)pyrrolidine-1-carboxylate (23-4, 400 mg, 1.64 mmol, 1.0 eq.) in THF (2 mL) was added BH₃-THF (4 mL) in portions at room temperature. The resulting mixture was stirred over night at 25° C. Desired product could be detected by LCMS. The resulting mixture was concentrated under reduced pressure. This resulted in tert-butyl (3S)-3-(2-aminoethoxy)pyrrolidine-1-carboxylate (200 mg, 53.0%) as a light yellow oil. MS: m/z: Calc'd for C₁₁H₂₂N₂O₃ [M+H]⁺231. Found, 231.

### Step 4 and 5

The title compound was prepared in 28.2% overall yield as a white solid according to Substitution reaction for synthesis of Carbamate; General Procedure I using tert-butyl (3S)-3-(2-aminoethoxy)pyrrolidine-1-carboxylate (23-5) in STEP 4; Boc Deprotection; General Procedure II in STEP 5. MS: m/z Calc'd for C₁₇H₂₂N₄O₄ [M+H]⁺380. Found, 380. ¹H NMR (400 MHz, Methanol-d₄) δ 7.28-7.20 (m, 2H), 6.97-6.90 (m, 2H), 4.97 (d, J=3.4 Hz, 1H), 4.38 (d, J=4.2 Hz, 1H), 4.34 (t, J=4.4 Hz, 1H), 4.18-4.14 (m, 1H), 3.80 (s, 3H), 3.62-3.58 (m, 3H), 3.49-3.35 (m, 5H), 3.31-3.17 (m, 2H), 3.12 (dd, J=14.2, 6.9 Hz, 1H), 2.98 (dd, J=14.2, 8.6 Hz, 1H), 2.29-2.18 (m, 1H), 2.14-2.04 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 18% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-[2-(2-nitroimidazol-1-yl)ethyl]carbamate (V188085)

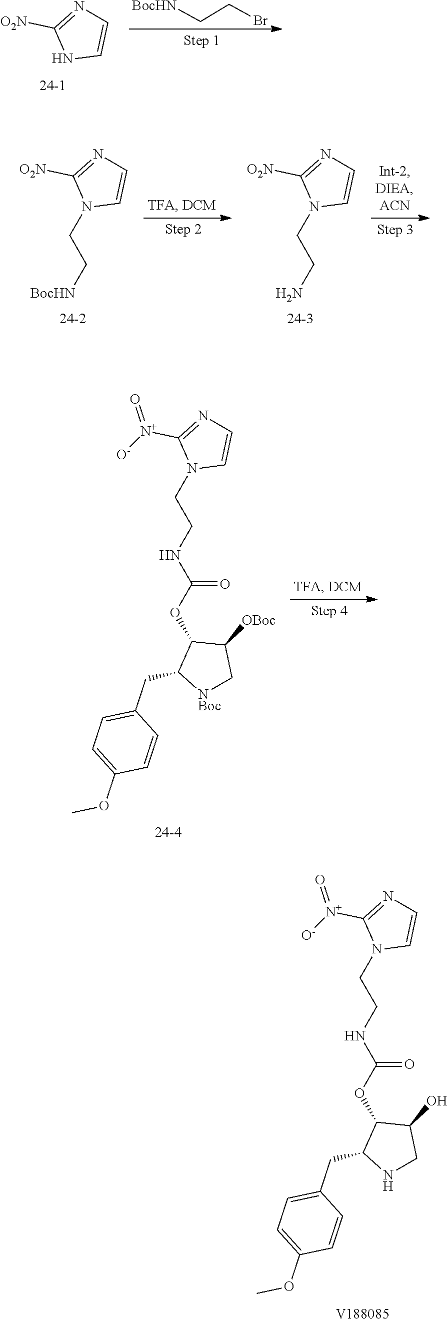

Step 1: To a stirred mixture of 2-nitroimidazole (24-1, 500 mg, 4.42 mmol, 1 eq.) and tert-butyl N-(2-bromoethyl)carbamate (1189.1 mg, 5.30 mmol, 1.2 eq.) in DMF (5 mL) was added K₂CO₃ (1833.3 mg, 13.26 mmol, 3 eq.) at room temperature. The resulting mixture was stirred at room temperature for overnight. Upon completion, concentrated, the residue was purified by reversed-phase flash chromatography to afford tert-butyl N-[2-(2-nitroimidazol-1-yl)ethyl]carbamate (800 mg) as a yellow oil. MS: m/z: Calc'd for C₁₀H₁₆N₄O₄ [M+H]⁺257. found 257.

Step 2: To a stirred mixture of tert-butyl N-[2-(2-nitroimidazol-1-yl)ethyl]carbamate (24-2, 800 mg, 3.12 mmol) in DCM (5 mL) was added TFA (1 mL) at room temperature. The resulting mixture was stirred at room temperature for 2h. Upon completion, concentrated to afford 2-(2-nitroimidazol-1-yl)ethanamine (420 mg, 86.1%) as a yellow oil.

### Step 3 and 4

The title compound was prepared in 38.6% overall yield as a yellow solid according to Substitution reaction for synthesis of Carbamate; General Procedure I using 2-(2-nitroimidazol-1-yl)ethanamine (24-3) in STEP 3; Boc Deprotection; General Procedure II in STEP 4. MS: m/z Calc'd for C₁₈H₂₃N₅O₆ [M+H]⁺406. found 406. ¹H NMR (400 MHz, Methanol-d₄) δ 7.46 (d, J=1.2 Hz, 1H), 7.25-7.17 (m, 2H), 7.14 (d, J=1.1 Hz, 1H), 6.97-6.89 (m, 2H), 4.84 (d, J=3.5 Hz, 1H), 4.65-4.52 (m, 2H), 4.33 (d, J=4.1 Hz, 1H), 4.13-4.01 (m, 1H), 3.80 (s, 3H), 3.67-3.52 (m, 1H), 3.59 (dd, J=6.8, 4.5 Hz, 1H), 3.56-3.47 (m, 1H), 3.20 (d, J=12.7 Hz, 1H), 3.05 (dd, J=14.3, 6.9 Hz, 1H), 2.89 (dd, J=14.3, 8.7 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 31% B in 10 min; Wave Length: 254 nm/220 nm; RT1(min): 9.78.

### Synthetic Procedure of (2R,3S,4S)-4-hydroxy-2-[(4-methoxyphenyl)methyl]pyrrolidin-3-yl N-[2-(2-aminoimidazole-1-yl)ethyl]carbamate (V188086)

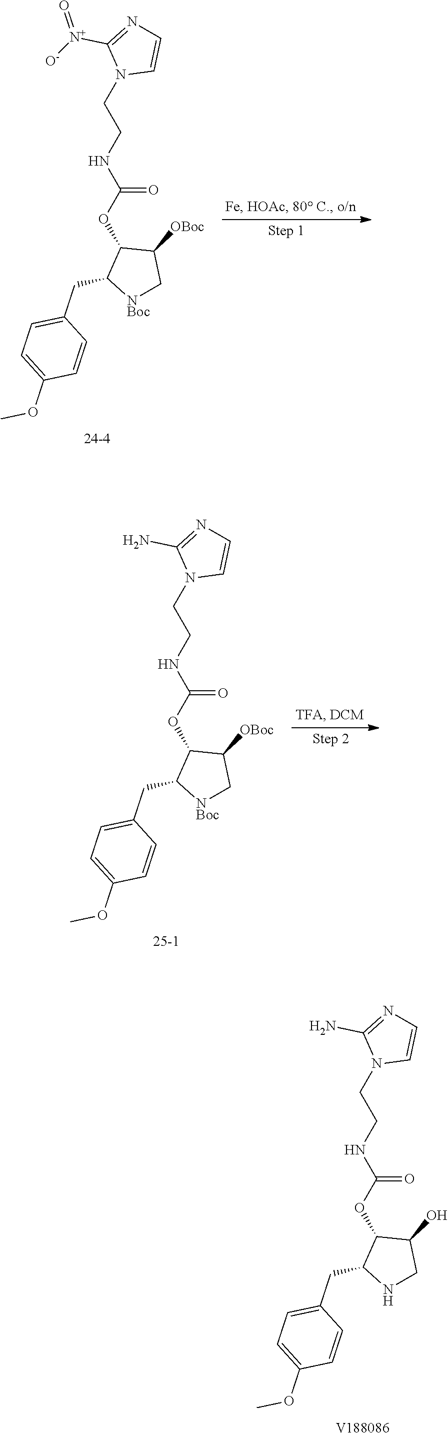

Step 1: To a stirred mixture of tert-butyl (2R,3S,4S)-4-[(tert-butoxycarbonyl)oxy]-2-[(4-methoxyphenyl)methyl]-3-({[2-(2-nitroimidazol-1-yl)ethyl]carbamoyl}oxy)pyrrolidine-1-carboxylate (24-4, 300 mg, 0.49 mmol, 1 eq.) in HOAc (10 mL) was added Fe (165.9 mg, 2.97 mmol, 6 eq.) at room temperature under nitrogen atmosphere. The resulting mixture was stirred at 80° C. for overnight. The resulting mixture was filtered, the filter cake was washed with HOAc (10 mL) (1×2 mL). The filtrate was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 254 nm. to afford tert-butyl (2R,3S,4S)-3-({[2-(2-aminoimidazole-1-yl)ethyl]carbamoyl}oxy)-4-[(tert-butoxycarbonyl)oxy]-2-[(4-methoxyphenyl)methyl]pyrrolidine-1-carboxylate (150 mg) as a brown oil. MS: m/z: Calc'd for C₂₈H₄₁N₅O₈ [M+H]⁺576. found 576.

Step 2: The title compound was prepared in 58.7% yield as a brown solid according to Boc Deprotection; General Procedure II. MS: m/z Calc'd for C₂₈H₄₁N₅O₈ [M+H]⁺376. found 376. ¹H NMR (400 MHz, Methanol-d₄) δ 7.19-7.09 (m, 2H), 6.89-6.79 (m, 2H), 6.64 (d, J=1.8 Hz, 1H), 6.54 (d, J=1.8 Hz, 1H), 4.62 (d, J=3.6 Hz, 1H), 4.18-4.11 (m, 1H), 3.95-3.84 (m, 2H), 3.77 (s, 3H), 3.56-3.42 (m, 1H), 3.45-3.32 (m, 2H), 3.41 (s, 1H), 2.87-2.70 (m, 3H).

Prep-HPLC purification conditions: Column: WelFlash C18, Regular C18 20-40 m, 120g; Mobile Phase A: Water (10 mmol/L NH4HCO3), Mobile Phase B: ACN; Flow rate: 60 mL/min; Gradient: 2% B to 25% B in 30 min; Wave Length: 254 nm/220 nm; RT1(min): 26.34.

### Synthetic Procedure of (2R,3S,4S)-4-hydroxy-2-[(4-methoxyphenyl)methyl]pyrrolidin-3-yl N-[2-(2-methylimidazol-1-yl)ethyl]carbamate (V188087)

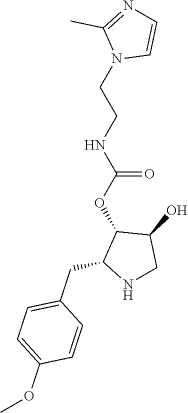 [SMILES: COc1ccc(C[C@H]2NC[C@H](O)[C@H]2OC(=O)NCCn2ccnc2C)cc1]

The title compound was prepared in 39.2% overall yield as a yellow oil according to Substitution reaction for synthesis of Carbamate; General Procedure I using 2-(2-methylimidazol-1-yl)ethanamine in STEP 1; Boc Deprotection; General Procedure II in STEP 2. MS: m/z Calc'd for C₁₉H₂₆N₄O₄[M+H]⁺375, Found 375. ¹H NMR (400 MHz, Methanol-d₄) δ 7.50 (d, J=2.1 Hz, 1H), 7.41 (d, J=2.2 Hz, 1H), 7.25-7.18 (m, 2H), 6.97-6.89 (m, 2H), 4.91 (s, 1H), 4.31 (s, 3H), 4.18-4.09 (m, 1H), 3.80 (s, 3H), 3.59-3.57 (m, 3H), 3.20 (d, J=12.7 Hz, 1H), 3.06 (dd, J=14.2, 7.0 Hz, 1H), 2.95 (dd, J=14.2, 8.6 Hz, 1H), 2.69 (s, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-[2-(2-oxo-3H-imidazol-1-yl)ethyl]carbamate (V188088)

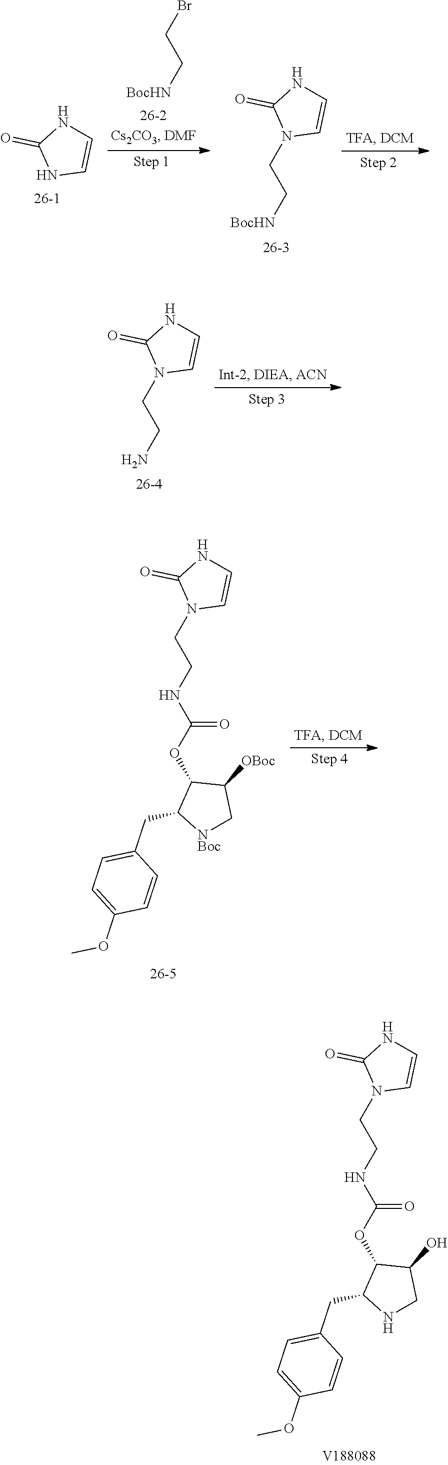

Step 1: To a stirred solution of 4-imidazolin-2-one (26-1, 300 mg, 3.56 mmol, 1 eq.) in DMF (5 mL) were added Cs₂CO₃ (3487.6 mg, 10.70 mmol, 3 eq.) and tert-butyl N-(2-bromoethyl)carbamate (26-2, 1599.2 mg, 7.13 mmol, 2 eq.) at 0° C. The resulting mixture was stirred at room temperature for overnight. The resulting mixture was concentrated under reduced pressure. The crude product was purified by reverse phase flash with the following conditions (NH₄HCO₃/H₂O) to afford tert-butyl N-[2-(2-oxo-3H-imidazol-1-yl)ethyl]carbamate (280 mg, 34.5%) as a light yellow oil. MS: m/z: Calc'd for C₁₀H₁₇N₃O₃[2M+H]⁺455. Found, 455.

Step 2: To a stirred solution of tert-butyl N-[2-(2-oxo-3H-imidazol-1-yl)ethyl]carbamate (26-3, 160 mg, 0.70 mmol) in DCM (3 mL) were added TFA (0.6 mL) at 0° C. The resulting mixture was stirred at room temperature for 2h. Upon completion, the resulting mixture was concentrated under reduced pressure to afford 1-(2-aminoethyl)-3H-imidazol-2-one as a light yellow oil. MS: m/z: Calc'd for C₅H₉N₃O[M+H]⁺128. Found, 128.

Step 3 and 4: The title compound was prepared in 68.1% overall yield as a light yellow semi-solid according to Substitution reaction for synthesis of Carbamate; General Procedure I using 1-(2-aminoethyl)-3H-imidazol-2-one (26-4) in STEP 3; Boc Deprotection; General Procedure II in STEP 4. MS: m/z Calc'd for C₁₈H₂₄N₄O₅ [M+H]⁺377. Found, 377. ¹H NMR (400 MHz, Methanol-d₄) δ 7.28-7.19 (m, 2H), 6.97-6.89 (m, 2H), 6.53-6.46-6.38 (m, 2H), 4.90 (d, J=3.5 Hz, 1H), 4.37 (dd, J=11.6, 4.3 Hz, 1H), 4.18-4.08 (m, 1H), 3.80-3.71 (m, 5H), 3.58 (dd, J=12.7, 4.4 Hz, 1H), 3.44 (t, J=5.8 Hz, 2H), 3.20 (d, J=12.6 Hz, 1H), 3.09 (dd, J=14.3, 7.1 Hz, 1H), 2.94 (dd, J=14.1, 8.5 Hz, 1H).

Prep-HPLC purification conditions: Column: Welch Ultimate AQ-C18 50*250, 10 μm; Mobile Phase A: Water (0.05% TFA), Mobile Phase B: ACN; Flow rate: 60 mL/min; Gradient: 2% B to 35% B in 15 min; Wave Length: 254 nm/220 nm; RT1(min): 16.8.

### Synthetic Procedure of (2R,3S,4S)-4-hydroxy-2-[(4-methoxyphenyl)methyl]pyrrolidin-3-yl N-[2-(1,3-benzodiazol-1-yl)ethyl]carbamate (V188090)

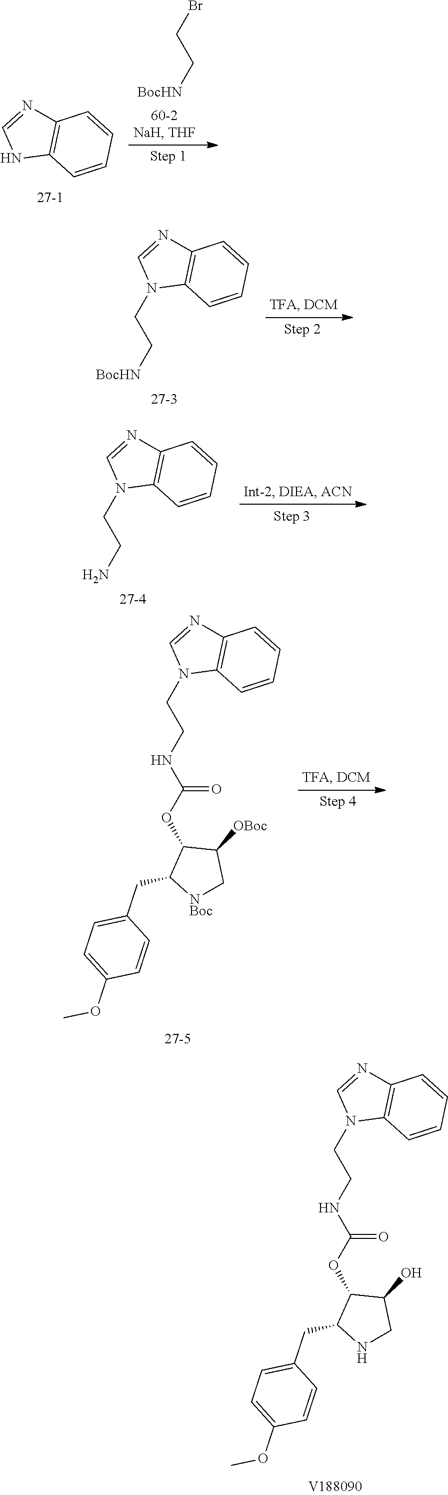

Step 1: To a stirred solution of NaH (81.2 mg, 3.38 mmol, 2 eq.) in THF (5 mL) was added benzimidazole (27-1, 200 mg, 1.69 mmol, 1 eq.) at 0° C. The resulting mixture was stirred for 30 min at 0° C. To the above mixture was added tert-butyl N-(2-bromoethyl)carbamate (27-2, 569.0 mg, 2.54 mmol, 1.5 eq.) at 0° C. The resulting mixture was stirred at room temperature for additional 8 h. The reaction was quenched with MeOH/H₂O at 0° C. The resulting mixture was extracted with EtOAc (3×50 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (1:2) to afford tert-butyl N-[2-(1,3-benzodiazol-1-yl)ethyl]carbamate (435 mg, 98.3%) as a white solid. MS: m/z: Calc'd for C₁₄H₁₉N₃O₂[M+H]⁺262. Found, 262.

Step 2: To a stirred solution of tert-butyl N-[2-(1,3-benzodiazol-1-yl)ethyl]carbamate (27-3, 430 mg, 1.64 mmol, 1 eq.) in DCM (5 mL) were added TFA (1 ml) at 0° C. The resulting mixture was stirred at room temperature for 2 h. The resulting mixture was concentrated under reduced pressure to afford (2R,3S,4S)-4-hydroxy-2-[(4-methoxyphenyl)methyl]pyrrolidin-3-yl N-[2-(hexahydropyrrolizin-7a-yl)ethyl]carbamate; trifluoroacetic acid as a yellow oil. MS: m/z: Calc'd for C₉H₁₁N₃[M+H]⁺162. Found, 162.

### Step 3 and 4

The title compound was prepared in 18.8% overall yield as a yellow oil according to Substitution reaction for synthesis of Carbamate; General Procedure I using 2-(1,3-benzodiazol-1-yl)ethanamine (27-4) in STEP 3; Boc Deprotection; General Procedure II in STEP 4. MS: m/z Calc'd for C₂₂H₂₆N₄O₄ [M+H]⁺411. Found, 411. ¹H NMR (400 MHz, Methanol-d₄) δ 9.46 (s, 1H), 8.00 (d, J=8.1 Hz, 1H), 7.85 (d, J=8.0 Hz, 1H), 7.71-7.58 (m, 2H), 7.14 (d, J=8.3 Hz, 2H), 6.89 (d, J=8.3 Hz, 2H), 4.81 (s, 3H), 4.03-4.01 (m, 1H), 3.96 (d, J=4.1 Hz, 1H), 3.84 (s, 5H), 3.11 (d, J=12.6 Hz, 1H), 2.89 (dd, J=14.2, 6.7 Hz, 1H), 2.80 (dd, J=14.2, 8.7 Hz, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 28% B in 10 min; Wave Length: 254 nm/220 nm; RT1(min): 8.62.

### Synthetic Procedure of (2R,3S,4S)-4-hydroxy-2-[(4-methoxyphenyl)methyl]pyrrolidin-3-yl N-{[(3S,4S)-4-hydroxypyrrolidin-3-yl]methyl}carbamate (V188093)

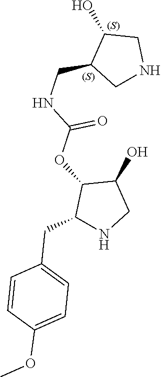

The title compound was prepared in 73.5% overall yield as a light yellow semi-solid according to Substitution reaction for synthesis of Carbamate; General Procedure I using tert-butyl (3S,4R)-3-(aminomethyl)-4-hydroxypyrrolidine-1-carboxylate in STEP 1; Boc Deprotection; General Procedure II in STEP 2. MS: m/z Calc'd for C₁₈H₂₇N₃O₅[M+H]⁺366, Found 366. ¹H NMR (400 MHz, Methanol-d₄) δ 7.24 (d, J=8.4 Hz, 2H), 6.97-6.90 (m, 2H), 4.94 (d, J=3.4 Hz, 1H), 4.39 (d, J=4.3 Hz, 1H), 4.33 (dd, J=5.3, 2.8 Hz, 1H), 4.16 (dd, J=7.8, 3.4 Hz, 1H), 3.80 (s, 3H), 3.65-3.55 (m, 2H), 3.45 (dd, J=12.4, 4.7 Hz, 1H), 3.32-3.06 (m, 6H), 3.01 (dd, J=14.1, 8.4 Hz, 1H), 2.53 (dd, J=9.4, 7.5, 4.0 Hz, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 30% B in 15 min; Wave Length: 254 nm/220 nm; RT1(min): 14.93.

### Synthetic Procedure of (2R,3S,4S)-4-hydroxy-2-[(4-methoxyphenyl)methyl]pyrrolidin-3-yl N-{[(3R,4R)-4-hydroxypyrrolidin-3-yl]methyl}carbamate (V188114)

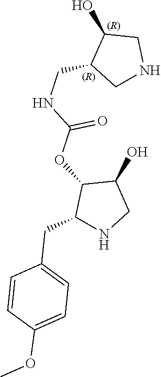

The title compound was prepared in 47.9% overall yield as a yellow oil according to Substitution reaction for synthesis of Carbamate; General Procedure I using tert-butyl (3S,4R)-3-(aminomethyl)-4-hydroxypyrrolidine-1-carboxylate in STEP 1; Boc Deprotection; General Procedure II in STEP 2. MS: m/z Calc'd for C₁₈H₂₇N₃O₅ [M+H]⁺366. found 366. ¹H NMR (400 MHz, Methanol-d₄) δ 7.29-7.20 (m, 2H), 6.97-6.89 (m, 2H), 4.98-4.92 (m, 1H), 4.39 (d, J=4.2 Hz, 1H), 4.33-4.21 (m, 1H), 4.17-4.02 (m, 1H), 3.79 (s, 3H), 3.64-3.54 (m, 2H), 3.43 (dd, J=12.3, 4.7 Hz, 1H), 3.23-3.12 (m, 4H), 3.16-3.06 (m, 2H), 2.99 (dd, J=14.1, 8.5 Hz, 1H), 2.53-2.42 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 7.93.

### Synthetic Procedure of (2R,3S,4S)-4-hydroxy-2-[(4-methoxyphenyl)methyl]pyrrolidin-3-yl N-{[(3R,4S)-4-hydroxypyrrolidin-3-yl]methyl}carbamate (V188115)

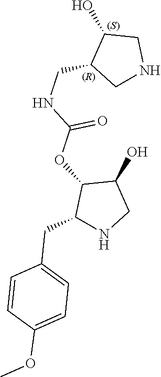

The title compound was prepared in 50.1% overall yield as a light yellow semi-solid according to Substitution reaction for synthesis of Carbamate; General Procedure I using tert-butyl (3S,4S)-3-(aminomethyl)-4-hydroxypyrrolidine-1-carboxylate in STEP 1; Boc Deprotection; General Procedure II in STEP 2. MS: m/z Calc'd for C₁₈H₂₇N₃O₅ [M+H]⁺366. Found, 366. ¹H NMR (400 MHz, Methanol-d₄) δ 7.27-7.20 (m, 2H), 6.97-6.89 (m, 2H), 4.97 (d, J=3.5 Hz, 1H), 4.45-4.43 (m, 1H), 4.39 (d, J=4.2 Hz, 1H), 4.19-4.15 (m, 1H), 3.80 (s, 3H), 3.59 (dd, J=12.6, 4.3 Hz, 1H), 3.52-3.37 (m, 3H), 3.37-3.34 (m, 2H), 3.22 (d, J=12.6 Hz, 1H), 3.16-3.07 (m, 2H), 2.99 (dd, J=14.1, 8.6 Hz, 1H), 2.56-2.48 (m, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 30% B in 15 min; Wave Length: 254 nm/220 nm; RT1(min): 14.32.

### Synthetic Procedure of (2R,3S,4S)-4-hydroxy-2-[(4-methoxyphenyl)methyl]pyrrolidin-3-yl N-(hexahydropyrrolizin-7a-ylmethyl)carbamate (V188134)

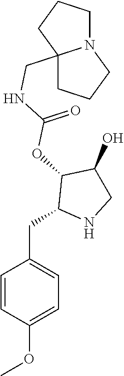 [SMILES: COc1ccc(C[C@H]2NC[C@H](O)[C@H]2OC(=O)NCC23CCCN2CCC3)cc1]

The title compound was prepared in 50.1% overall yield as a light yellow oil according to Substitution reaction for synthesis of Carbamate; General Procedure I using 1-(hexahydropyrrolizin-7a-yl)methanamine in STEP 1; Boc Deprotection; General Procedure II in STEP 2. MS: m/z Calc'd for C₂₁H₃₁N₃O₄[M+H]⁺390. Found, 390. ¹H NMR (400 MHz, Methanol-d₄) δ 7.29-7.20 (m, 2H), 6.97-6.88 (m, 2H), 4.98 (d, J=3.4 Hz, 1H), 4.41 (d, J=4.3 Hz, 1H), 4.20-4.18 (m, 1H), 3.80 (s, 3H), 3.62-3.53 (m, 5H), 3.31-3.20 (m, 3H), 3.12 (dd, J=14.2, 7.3 Hz, 1H), 3.02 (dd, J=14.1, 8.5 Hz, 1H), 2.20-2.18 (m, 4H), 2.06-2.04 (m, 4H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 30% B in 15 min; Wave Length: 254 nm/220 nm; RT1(min): 16.53.

### Synthetic Procedure of (2R,3S,4S)-4-hydroxy-2-[(4-methoxyphenyl)methyl]pyrrolidin-3-yl N-[2-(hexahydropyrrolizin-7a-yl)ethyl]carbamate (V188135)

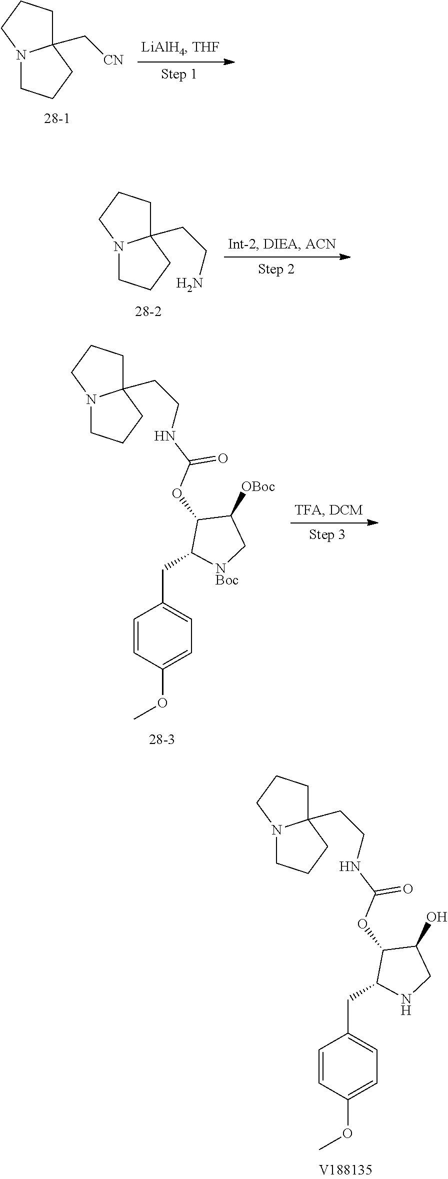

Step 1: To a stirred solution of 2-(hexahydropyrrolizin-7a-yl)acetonitrile (28-1, 150 mg, 0.99 mmol, 1 eq.) in THF (4 mL) were added LiAlH₄ (75.7 mg, 1.99 mmol, 2 eq.) at 0° C. The resulting mixture was stirred for overnight at 80° C. Upon completion, the reaction was quenched with Na₂SO₄·10H₂O, filtered, washed with. The filtrate was concentrated under reduced pressure to afford 2-(hexahydropyrrolizin-7a-yl)ethanamine (150 mg, 97.3%) as a yellow oil. MS: m/z: Calc'd for C₉H₁₈N₂ [M+H]⁺155. Found, 155.

Step 2 and 3: The title compound was prepared in 30.3% overall yield as a white solid according to Substitution reaction for synthesis of Carbamate; General Procedure I using 2-(hexahydropyrrolizin-7a-yl)ethanamine (28-2) in STEP 2; Boc Deprotection; General Procedure II in STEP 3. MS: m/z Calc'd for C₂₂H₃₃N₃O₄ [M+H]⁺404. Found, 404. ¹H NMR (400 MHz, Methanol-d₄) δ 7.28-7.20 (m, 2H), 6.96-6.90 (m, 2H), 4.97 (d, J=3.9 Hz, 1H), 4.38 (d, J=4.3 Hz, 1H), 4.21-4.12 (m, 1H), 3.80 (s, 3H), 3.59-3.57 (m, 3H), 3.34-3.26 (m, 2H), 3.22-3.16 (m, 3H), 3.11 (dd, J=14.2, 7.1 Hz, 1H), 2.98 (dd, J=14.1, 8.5 Hz, 1H), 2.27-1.97 (m, 10H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min mL/min; Gradient: 2% B to 35% B in 15 min; Wave Length: 254 nm/220 nm; RT1(min): 15.97.

### Synthetic Procedure of (2R,3S,4S)-4-hydroxy-2-[(4-methoxyphenyl)methyl]pyrrolidin-3-yl N-{[4-(1H-imidazol-2-yl)phenyl]methyl}carbamate (V188144)

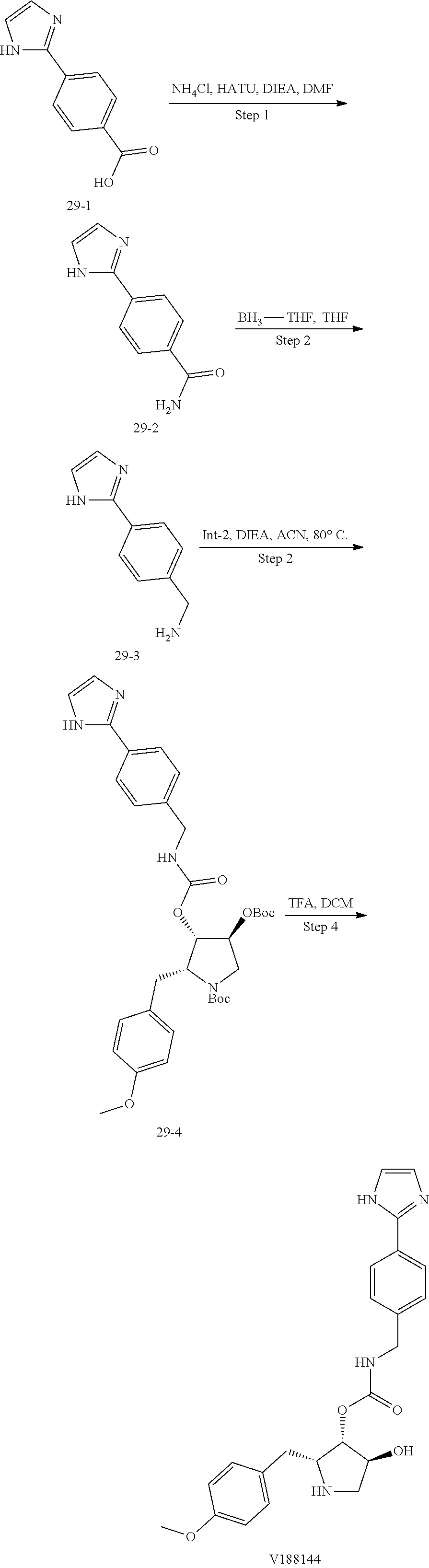

Step 1: To a stirred solution of 4-(1H-imidazol-2-yl)benzoic acid (29-1, 150 mg, 0.79 mmol, 1 eq.) and NH₄Cl (85.2 mg, 1.59 mmol, 2 eq.) in DMF (3 mL) was added HATU (454.6 mg, 1.19 mmol, 1.5 eq.) and DIEA (309.0 mg, 2.39 mmol, 3 eq.) at 0° C. The resulting mixture was stirred at room temperature for overnight. Upon completion, the crude product (150 mg) was purified by Prep-HPLC to afford 4-(1H-imidazol-2-yl)benzamide (150 mg, 97.4%) as a light yellow oil. MS: m/z: Calc'd for C₁₀H₉N₃O [M+H]⁺188. Found, 188.

Step 2: To a stirred solution of 4-(1H-imidazol-2-yl)benzamide (29-2, 225 mg, 1.20 mmol, 1 eq.) in THF (4 mL) were added BH₃-THF (8 mL) at 0° C. The resulting mixture was stirred at room temperature for overnight. Upon completion, the resulting mixture was concentrated under reduced pressure to afford 4-(1H-imidazol-2-yl)benzamide (200 mg, 96.1%) as a light yellow oil. MS: m/z: Calc'd for C₁₀H₁₁N₃[M+H]⁺174. Found, 174.

### Step 3 and 4

The title compound was prepared in 41.2% overall yield as a white solid according to Substitution reaction for synthesis of Carbamate; General Procedure I using 1-[4-(1H-imidazol-2-yl)phenyl]methanamine (29-3) in STEP 3; Boc Deprotection; General Procedure II in STEP 4. MS: m/z Calc'd for C23H₂₆N₄O₄ [M+H]⁺423. Found, 423. ¹H NMR (400 MHz, Methanol-d₄) δ 7.97-7.90 (m, 2H), 7.64 (d, J=9.4 Hz, 4H), 7.26-7.19 (m, 2H), 6.93-6.85 (m, 2H), 5.00-4.95 (m, 1H), 4.51 (d, J=15.9 Hz, 2H), 4.47-4.38 (m, 1H), 4.18-4.16 (m, 1H), 3.78 (s, 3H), 3.60 (dd, J=12.6, 4.3 Hz, 1H), 3.24 (d, J=12.6 Hz, 1H), 3.15-3.07 (m, 1H), 2.99 (dd, J=14.1, 8.4 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-{[(1s,3s)-3-aminocyclobutyl]methyl}carbamate (V188147)

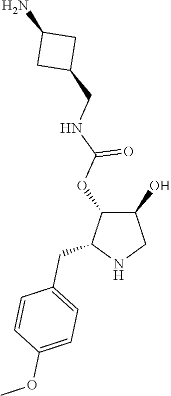 [SMILES: COc1ccc(C[C@H]2NC[C@H](O)[C@H]2OC(=O)NC[C@H]2C[C@@H](N)C2)cc1]

The title compound was prepared in 17.6% overall yield as a white semi-solid according to Substitution reaction for synthesis of Carbamate; General Procedure I using tert-butyl N-[(1s,3s)-3-(aminomethyl)cyclobutyl]carbamate in STEP 1; Boc Deprotection; General Procedure II in STEP 2. MS: m/z Calc'd for C₁₈H₂₇N₃O₄ [M+H]⁺350. Found, 350. ¹H NMR (400 MHz, Methanol-d₄) δ 7.23 (d, J=8.3 Hz, 2H), 6.93 (d, J=8.5 Hz, 2H), 4.94 (d, J=3.5 Hz, 1H), 4.37 (d, J=4.2 Hz, 1H), 4.18-4.14 (m, 1H), 3.80 (s, 3H), 3.72-7.64 (m, 1H), 3.58 (dd, J=12.6, 4.3 Hz, 1H), 3.31-3.18 (m, 3H), 3.11 (dd, J=14.1, 7.1 Hz, 1H), 2.98 (dd, J=14.1, 8.5 Hz, 1H), 2.52-2.46 (m, 2H), 2.43-2.29 (m, 1H), 1.94-1.86 (m, 2H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 25% B in 15 min; Wave Length: 254 nm/220 nm; RT1(min): 14.45.

### Synthetic Procedure of (2R,3S,4S)-4-hydroxy-2-[(4-methoxyphenyl)methyl]pyrrolidin-3-yl N-(1H-indazol-6-ylmethyl)carbamate (V188151)

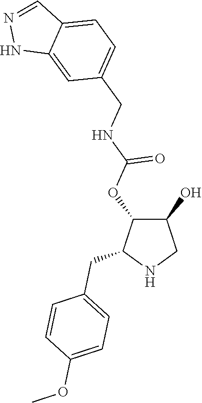 [SMILES: COc1ccc(C[C@H]2NC[C@H](O)[C@H]2OC(=O)NCc2ccc3cn[nH]c3c2)cc1]

The title compound was prepared in 15.5% overall yield as a white solid according to Substitution reaction for synthesis of Carbamate; General Procedure I using 1-(1H-indazol-6-yl)methanamine in STEP 1; Boc Deprotection; General Procedure II in STEP 2. MS: m/z Calc'd for C₂₁H₂₄N₄O₄ [M+H]⁺397. Found, 397. ¹H NMR (400 MHz, Methanol-d₄) δ 8.05 (s, 1H), 7.79 (d, J=8.3 Hz, 1H), 7.53 (s, 1H), 7.24-7.11 (m, 3H), 6.80-6.76 (m, 2H), 4.58-4.36 (m, 3H), 4.15-4.13 (m, 1H), 3.72 (d, J=3.5 Hz, 3H), 3.65-3.55 (m, 1H), 3.23 (d, J=12.6 Hz, 1H), 3.09 (dd, J=14.0, 7.9 Hz, 1H), 2.99 (dd, J=14.0, 7.5 Hz, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 15% B to 45% B in 15 min; Wave Length: 254 nm/220 nm; RT1(min): 14.18.

### Synthetic Procedure of (2R,3S,4S)-4-hydroxy-2-[(4-methoxyphenyl)methyl]pyrrolidin-3-yl N-{2-azabicyclo[2.1.1]hexan-4-ylmethyl}carbamate (V188152)

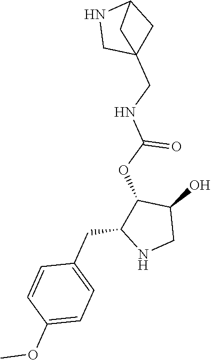 [SMILES: COc1ccc(C[C@H]2NC[C@H](O)[C@H]2OC(=O)NCC23CNC(C2)C3)cc1]

The title compound was prepared in 26.8% overall yield as a yellow semi-solid according to Substitution reaction for synthesis of Carbamate; General Procedure I using tert-butyl 4-(aminomethyl)-2-azabicyclo[2.1.1]hexane-2-carboxylate in STEP 1; Boc Deprotection; General Procedure II in STEP 2. MS: m/z Calc'd for C₁₉H₂₇N₃O₄ [M+H]⁺362. Found, 362. ¹H NMR (400 MHz, Methanol-d₄) δ 7.29-7.20 (m, 2H), 6.97-6.89 (m, 2H), 4.97 (d, J=3.5 Hz, 1H), 4.39 (d, J=4.2 Hz, 1H), 4.23-4.13 (m, 2H), 3.80 (s, 3H), 3.65-3.44 (m, 3H), 3.30-3.19 (m, 3H), 3.13 (dd, J=14.1, 7.2 Hz, 1H), 3.00 (dd, J=14.1, 8.5 Hz, 1H), 2.09-1.99 (m, 2H), 1.66-1.54 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 32% B in 15 min; Wave Length: 254 nm/220 nm; RT1(min): 15.95.

### Synthetic Procedure of (2R,3S,4S)-4-hydroxy-2-[(4-methoxyphenyl)methyl]pyrrolidin-3-yl N-[(1,1-dioxo-1lambda6-thiolan-3-yl)methyl]carbamate (V188154)

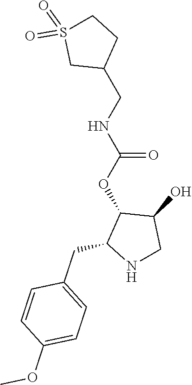 [SMILES: COc1ccc(C[C@H]2NC[C@H](O)[C@H]2OC(=O)NCC2CCS(=O)(=O)C2)cc1]

The title compound was prepared in 23.3% overall yield as a white solid according to Substitution reaction for synthesis of Carbamate; General Procedure I using 3-(aminomethyl)-1lambda6-thiolane-1,1-dione in STEP 1; Boc Deprotection; General Procedure II in STEP 2. MS: m/z Calc'd for C₁₇H₂₂N₄O₄ [M+H]⁺399. Found, 399. ¹H NMR (400 MHz, Methanol-d₄) δ 7.24 (dd, J=8.5, 1.7 Hz, 2H), 6.94 (dd, J=8.7, 1.9 Hz, 2H), 4.94-4.92 (m, 1H), 4.40 (d, J=3.8 Hz, 1H), 4.21-4.17 (m, 1H), 3.80 (s, 3H), 3.58 (dd, J=12.6, 4.3 Hz, 1H), 3.38 (dd, J=14.0, 6.0 Hz, 1H), 3.32-3.19 (m, 4H), 3.18-3.05 (m, 2H), 3.00 (dd, J=14.1, 8.2 Hz, 1H), 2.89-2.83 (m, 1H), 2.76-2.63 (m, 1H), 2.38-2.30 (m, 1H), 2.00-1.80 (m, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 40% B in 15 min; Wave Length: 254 nm/220 nm; RT1(min): 16.15.

### Synthetic Procedure of (2R,3S,4S)-4-hydroxy-2-[(4-methoxyphenyl)methyl]pyrrolidin-3-yl N-[(1R,5S,6S)-3-azabicyclo[3.1.0]hexan-6-ylmethyl]carbamate (V188161)

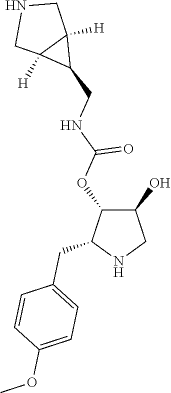 [SMILES: COc1ccc(C[C@H]2NC[C@H](O)[C@H]2OC(=O)NC[C@@H]2[C@@H]3CNC[C@@H]32)cc1]

The title compound was prepared in 29.7% overall yield as a light yellow oil according to Substitution reaction for synthesis of Carbamate; General Procedure I using tert-butyl (1R,5S,6S)-6-(aminomethyl)-3-azabicyclo[3.1.0]hexane-3-carboxylate in STEP 1; Boc Deprotection; General Procedure II in STEP 2. MS: m/z Calc'd for C₁₉H₂₇N₃O₄ [M+H]⁺362. found 362. ¹H NMR (400 MHz, Methanol-d₄) δ 7.24 (d, J=8.5 Hz, 2H), 6.97-6.89 (m, 2H), 4.97 (d, J=3.5 Hz, 1H), 4.39 (d, J=4.2 Hz, 1H), 4.18-4.05 (m, 1H), 3.80 (s, 3H), 3.73-3.62 (m, 2H), 3.60 (dd, J=12.6, 4.3 Hz, 1H), 3.41 (dd, J=12.7, 3.7 Hz, 2H), 3.25 (dd, J=12.4, 10.2 Hz, 3H), 3.12 (dd, J=14.2, 7.4 Hz, 1H), 3.00 (dd, J=14.1, 8.4 Hz, 1H), 2.18-2.07 (m, 2H), 1.47-1.37 (m, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 25% B in 15 min; Wave Length: 254 nm/220 nm; RT1(min): 16.75.

### Synthetic Procedure of (2R,3S,4S)-4-hydroxy-2-[(4-methoxyphenyl)methyl]pyrrolidin-3-yl N-(2,2-difluoro-3-hydroxypropyl)carbamate (V188164)

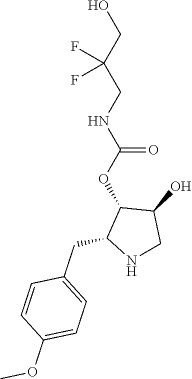 [SMILES: COc1ccc(C[C@H]2NC[C@H](O)[C@H]2OC(=O)NCC(F)(F)CO)cc1]

The title compound was prepared in 41.7% overall yield as a white solid according to Substitution reaction for synthesis of Carbamate; General Procedure I using tert-butyl (1R,5S,6S)-6-(aminomethyl)-3-azabicyclo[3.1.0]hexane-3-carboxylate in STEP 1; Boc Deprotection; General Procedure II in STEP 2. MS: m/z Calc'd for C₁₆H₂₂F₂N₂O₅[M+H]⁺361. Found, 361. ¹H NMR (400 MHz, Methanol-d₄) δ 7.28-7.20 (m, 2H), 6.97-6.89 (m, 2H), 4.99-4.93 (m, 1H), 4.40 (d, J=4.2 Hz, 1H), 4.18-4.16 (m, 1H), 3.80 (s, 3H), 3.79-3.64 (m, 3H), 3.64-3.54 (m, 1H), 3.38 (s, 1H), 3.23 (d, J=12.7 Hz, 1H), 3.13 (dd, J=14.3, 7.6 Hz, 1H), 2.98 (dd, J=14.0, 8.4 Hz, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 40% B in 15 min; Wave Length: 254 nm/220 nm; RT1(min): 16.2.

### Synthetic Procedure of (2R,3S,4S)-4-hydroxy-2-[(4-methoxyphenyl)methyl]pyrrolidin-3-yl N-(2,2-difluoro-2-phenylethyl)carbamate (V188165)

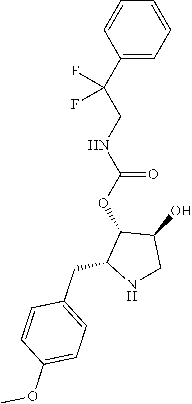 [SMILES: COc1ccc(C[C@H]2NC[C@H](O)[C@H]2OC(=O)NCC(F)(F)c2ccccc2)cc1]

The title compound was prepared in 35.1% overall yield as a white solid according to Substitution reaction for synthesis of Carbamate; General Procedure I using tert-butyl (1R,5S,6S)-6-(aminomethyl)-3-azabicyclo[3.1.0]hexane-3-carboxylate in STEP 1; Boc Deprotection; General Procedure II in STEP 2. MS: m/z Calc'd for C₂₁H₂₄F₂N₂O₄[M+H]⁺407. Found, 407. ¹H NMR (400 MHz, Methanol-d₄) δ 7.61-7.45 (m, 5H), 7.19 (d, J=8.2 Hz, 2H), 7.09 (s, 2H), 4.91 (d, J=8.2 Hz, 1H), 4.27 (d, J=4.2 Hz, 1H), 4.13-4.06 (m, 1H), 3.85 (d, J=11.8 Hz, 2H), 3.80 (s, 3H), 3.53 (dd, J=12.5, 4.3 Hz, 1H), 3.30 (s, 1H), 3.20 (d, J=12.6 Hz, 1H), 2.87 (dd, J=14.1, 8.3 Hz, 1H).

Prep-HPLC purification conditions: Column: Xselect CSH Prep Fluoro-Phenyl OBD C18 Column, 30*150 mm, 5 μm; Mobile Phase A: Water (0.1% TFA), Mobile Phase B: ACN; Flow rate: 60 mL/min; Gradient: 9% B to 34% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-{[(3R)-1-(1,3-thiazol-2-yl)pyrrolidin-3-yl]methyl}carbamate (V188170)

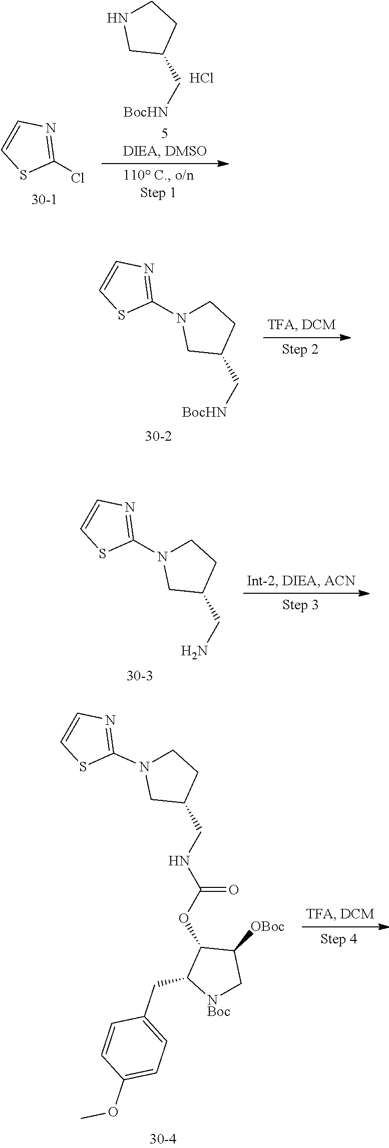

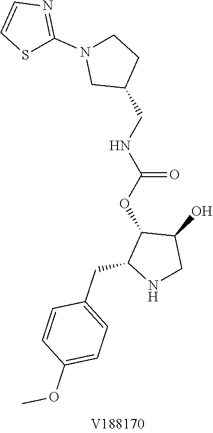 [SMILES: COc1ccc(C[C@H]2NC[C@H](O)[C@H]2OC(=O)NC[C@H]2CCN(c3nccs3)C2)cc1]

Step 1: To a stirred solution of 2-chloro-1,3-thiazole (30-1, 150 mg, 1.25 mmol, 1 eq.) and DIEA (486.4 mg, 3.76 mmol, 3 eq.) in DMSO (3 mL) was added tert-butyl N-[(3S)-pyrrolidin-3-ylmethyl]carbamate hydrochloride (593.9 mg, 2.50 mmol, 2 eq.) at room temperature. The resulting mixture was stirred at 110° C. for overnight. Upon completion, the mixture was purified by reversed phase flash to afford tert-butyl N-{[(3R)-1-(1,3-thiazol-2-yl)pyrrolidin-3-yl]methyl}carbamate (180 mg, 50.6%) as a light yellow solid. MS: m/z: Calc'd for C₁₃H₂₁N₃O₂S[M+H]⁺284. Found, 284.

Step 2: To a stirred solution of tert-butyl N-{[(3R)-1-(1,3-thiazol-2-yl)pyrrolidin-3-yl]methyl}carbamate (30-2, 175 mg, 0.61 mmol, 1 eq.) in DCM (3 mL) were added TFA (0.6 mL) at 0° C. The resulting mixture was stirred at room temperature for 2h. The resulting mixture was concentrated under reduced pressure to afford 1-[(3R)-1-(1,3-thiazol-2-yl)pyrrolidin-3-yl]methanamine (150 mg) as a light yellow solid. MS: m/z: Calc'd for C₈H₁₃N₃S[M+H]⁺184. Found, 184.

### Step 3 and 4

The title compound was prepared in 26.8% overall yield as a yellow oil according to Substitution reaction for synthesis of Carbamate; General Procedure I using 1-[(3R)-1-(1,3-thiazol-2-yl)pyrrolidin-3-yl]methanamine (30-3) in STEP 3; Boc Deprotection; General Procedure II in STEP 4. MS: m/z Calc'd for C₂₁H₂₈N₄O₄S [M+H]⁺433. Found, 433. ¹H NMR (400 MHz, Methanol-d₄) δ 7.32 (d, J=4.3 Hz, 1H), 7.28-7.20 (m, 2H), 6.96-6.85 (m, 3H), 4.96 (d, J=2.8 Hz, 1H), 4.40 (d, J=4.0 Hz, 1H), 4.19-4.17 (m, 1H), 3.78 (s, 3H), 3.77-3.65 (m, 2H), 3.61-3.54 (m, 2H), 3.32-3.20 (m, 2H), 3.12 (dd, J=14.1, 7.3 Hz, 1H), 2.98-2.89 (m, 1H), 2.83 (dd, J=14.1, 8.5 Hz, 1H), 2.75 (d, J=7.0 Hz, 1H), 2.38-2.35 (m, 1H), 2.32-2.30 (m, 1H), 2.08-1.95 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 26% B in 10 min; Wave Length: 254 nm/220 nm; RT1(min): 8.78.

### Synthetic Procedure of (2R,3S,4S)-4-hydroxy-2-[(4-methoxyphenyl)methyl]pyrrolidin-3-yl N-{[(3R)-1-(pyrimidin-2-yl)pyrrolidin-3-yl]methyl}carbamate (V188171)

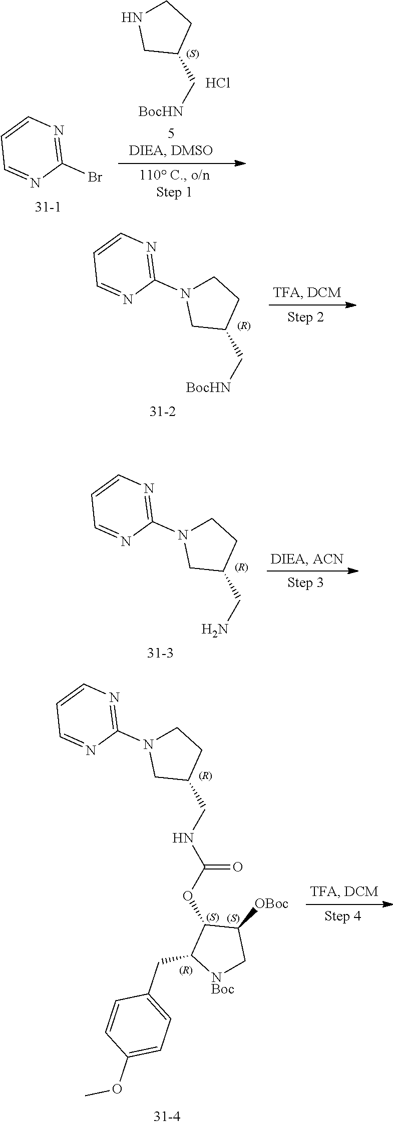

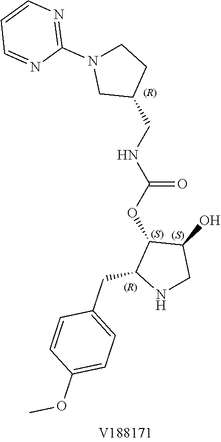

Step 1: To a stirred solution of pyrimidine, 2-bromo- (150 mg, 0.94 mmol, 1 eq.) and DIEA (31-1, 365.8 mg, 2.82 mmol, 3 eq.) in DMSO (3 mL) was added tert-butyl N-[(3S)-pyrrolidin-3-ylmethyl]carbamate hydrochloride (446.7 mg, 1.88 mmol, 2 eq.) at room temperature. The resulting mixture was stirred at 110° C. for overnight. Upon completion, the mixture was purified by reversed phase flash to afford tert-butyl N-{[(3R)-1-(pyrimidin-2-yl)pyrrolidin-3-yl]methyl}carbamate (200 mg, 76.1%) as a light yellow solid. MS: m/z: Calc'd for C₁₄H₂₂N₄O₂[M+H]⁺279. Found, 279.

Step 2: To a stirred solution of tert-butyl N-{[(3R)-1-(pyrimidin-2-yl)pyrrolidin-3-yl]methyl}carbamate (31-2, 195 mg, 0.70 mmol) in DCM (3 mL) was added TFA (0.6 mL) at 0° C. The resulting mixture was stirred at room temperature for 2 h. The resulting mixture was concentrated under reduced pressure to afford 1-[(3R)-1-(pyrimidin-2-yl)pyrrolidin-3-yl]methanamine (150 mg) as a light yellow solid. MS: m/z: Calc'd for C₉H₁₄N₄[M+H]⁺179. Found, 179.

### Step 3 and 4

The title compound was prepared in 34.6% overall yield as a light yellow semi-solid according to Substitution reaction for synthesis of Carbamate; General Procedure I using 1-[(3R)-1-(pyrimidin-2-yl)pyrrolidin-3-yl]methanamine (64-3) in STEP 3; Boc Deprotection; General Procedure II in STEP 4. MS: m/z Calc'd for C₂₂H₂₉N₅O₄ [M+H]⁺428. Found, 428. ¹H NMR (400 MHz, Methanol-d₄) δ 8.43 (d, J=5.1 Hz, 2H), 7.24 (d, J=8.5 Hz, 2H), 6.96-6.87 (m, 2H), 6.75 (t, J=5.0 Hz, 1H), 4.97-4.92 (m, 1H), 4.43-4.38 (m, 1H), 4.18-4.16 (m, 1H), 3.85-3.71 (m, 1H), 3.69 (s, 4H), 3.67-3.54 (m, 2H), 3.43-3.32 (m, 2H), 3.24 (dd, J=13.5, 7.2 Hz, 2H), 3.12 (dd, J=14.1, 7.4 Hz, 1H), 2.98 (dd, J=14.1, 8.4 Hz, 1H), 2.63 (d, J=7.0 Hz, 1H), 2.22-2.20 (m, 1H), 1.89-1.85 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 27% B in 10 min; Wave Length: 254 nm/220 nm; RT1(min): 9.

### Synthetic Procedure of (2R,3S,4S)-4-hydroxy-2-[(4-methoxyphenyl)methyl]pyrrolidin-3-yl N-(piperidin-4-ylmethyl)carbamate (V188172)

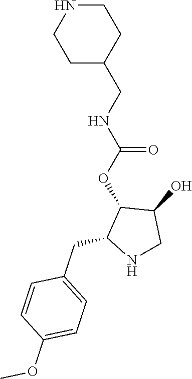 [SMILES: COc1ccc(C[C@H]2NC[C@H](O)[C@H]2OC(=O)NCC2CCNCC2)cc1]

The title compound was prepared in 44.2% overall yield as a white solid according to Substitution reaction for synthesis of Carbamate; General Procedure I using tert-butyl 4-(aminomethyl)piperidine-1-carboxylate in STEP 1; Boc Deprotection; General Procedure II in STEP 2. MS: m/z Calc'd for C₁₉H₂₉N₃O₄ [M+H]⁺364. found 364. ¹H NMR (400 MHz, Methanol-d₄) δ 7.28-7.20 (m, 2H), 6.96-6.88 (m, 2H), 4.98-4.92 (m, 1H), 4.41-4.35 (m, 1H), 4.17-4.05 (m, 1H), 3.80 (s, 3H), 3.59 (dd, J=12.6, 4.3 Hz, 1H), 3.49-3.39 (m, 2H), 3.22 (d, J=12.6 Hz, 1H), 3.19-3.06 (m, 3H), 3.00-2.89 (m, 3H), 2.03-1.93 (m, 2H), 1.86-1.74 (m, 1H), 1.53-1.36 (m, 2H)

Prep-HPLC purification conditions: Column: Welch Ultimate AQ-C18 50*250, 10 μm; Mobile Phase A: Water (0.05% TFA), Mobile Phase B: ACN; Flow rate: 60 mL/min; Gradient: 2% B to 30% B in 15 min; Wave Length: 254 nm/220 nm; RT1(min): 16.47.

### Synthetic Procedure of (2R,3S,4S)-4-hydroxy-2-[(4-methoxyphenyl)methyl]pyrrolidin-3-yl N-{2-[4-(fluorosulfonyl)phenyl]ethyl}carbamate (V188175)

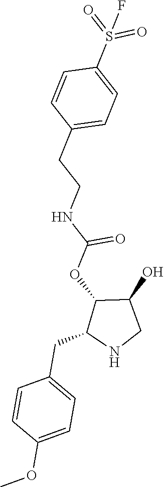 [SMILES: COc1ccc(C[C@H]2NC[C@H](O)[C@H]2OC(=O)NCCc2ccc(S(=O)(=O)F)cc2)cc1]

The title compound was prepared in 44.2% overall yield as a white solid according to Substitution reaction for synthesis of Carbamate; General Procedure I using aebsf in STEP 1; Boc Deprotection; General Procedure II in STEP 2. MS: m/z Calc'd for C₂₁H₂₅FN₂O₆S [M+H]⁺453, found 453. ¹H NMR (400 MHz, Methanol-d₄) δ 8.04-7.97 (m, 2H), 7.63 (d, J=8.2 Hz, 2H), 7.23-7.15 (m, 2H), 6.96-6.88 (m, 2H), 4.94-4.89 (m, 1H), 4.35-4.30 (m, 1H), 4.13-4.02 (m, 1H), 3.80 (s, 3H), 3.60-3.43 (m, 3H), 3.20 (d, J=12.6 Hz, 1H), 3.04-2.94 (m, 3H), 2.89 (dd, J=14.1, 8.5 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 17% B to 47% B in 10 min; Wave Length: 254 nm/220 nm; RT1(min): 9.05.

### Synthetic Procedure of (2R,3S,4S)-4-hydroxy-2-[(4-methoxyphenyl)methyl]pyrrolidin-3-yl N-{2-[4-(fluorosulfonyl)phenyl]ethyl}carbamate (V188205)

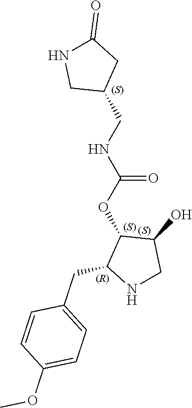

The title compound was prepared in 48.8% overall yield as a white solid according to Substitution reaction for synthesis of Carbamate; General Procedure I using aebsf in STEP 1; Boc Deprotection; General Procedure II in STEP 2. MS: m/z Calc'd for C₂₈H₄₁N₃O₉ [M+H]⁺364, found 364. ¹H NMR (400 MHz, Methanol-d₄) δ 7.27-7.19 (m, 2H), 6.97-6.90 (m, 2H), 4.94 (d, J=3.1 Hz, 1H), 4.39 (d, J=4.1 Hz, 1H), 4.17-4.05 (m, 1H), 3.80 (s, 3H), 3.66-3.48 (m, 2H), 3.26 (d, J=6.8 Hz, 2H), 3.23-3.05 (m, 3H), 3.02-2.91 (m, 1H), 2.78-2.65 (m, 1H), 2.47 (dd, J=17.0, 8.9 Hz, 1H), 2.15 (dd, J=17.1, 6.2 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 23% B in 10 min; Wave Length: 254 nm/220 nm; RT1(min): 8.87.

### Synthetic Procedure of (2R,3S,4S)-4-hydroxy-2-[(4-methoxyphenyl)methyl]pyrrolidin-3-yl N-[(6-oxo-1H-pyridin-3-yl)methyl]carbamate (V188206)

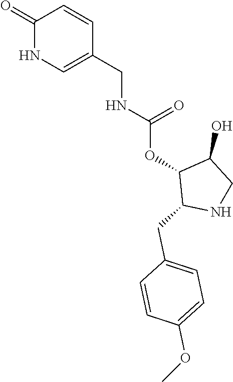 [SMILES: COc1ccc(C[C@H]2NC[C@H](O)[C@H]2OC(=O)NCc2ccc(=O)[nH]c2)cc1]

The title compound was prepared in 34.5% overall yield as a yellow solid according to Substitution reaction for synthesis of Carbamate; General Procedure I using 5-(aminomethyl)-1H-pyridin-2-one in STEP 1; Boc Deprotection; General Procedure II in STEP 2. MS: m/z Calc'd for C₁₉H₂₃N₃O₅[M+H]⁺374. Found, 374. ¹H NMR (400 MHz, Methanol-d₄) δ 7.64 (dd, J=9.4, 2.6 Hz, 1H), 7.42 (d, J=2.6 Hz, 1H), 7.22-7.14 (m, 2H), 6.92-6.84 (m, 2H), 6.58 (d, J=9.4 Hz, 1H), 4.96-4.91 (m, 1H), 4.39 (d, J=4.2 Hz, 1H), 4.21-4.09 (d, J=15.0 Hz, 3H), 3.79 (s, 3H), 3.56 (dd, J=12.7, 4.3 Hz, 1H), 3.23 (d, J=12.6 Hz, 1H), 3.09 (dd, J=14.0, 7.6 Hz, 1H), 2.96 (dd, J=14.0, 8.2 Hz, 1H).

Prep-HPLC purification conditions: Column: Welch Ultimate AQ-C18 50*250, 10 μm; Mobile Phase A: Water (0.05% TFA), Mobile Phase B: ACN; Flow rate: 60 mL/min; Gradient: 2% B to 40% B in 15 min; Wave Length: 254 nm/220 nm; RT1(min): 15.75.

### Synthetic Procedure of (2R,3S,4S)-4-hydroxy-2-[(4-methoxyphenyl)methyl]pyrrolidin-3-yl N-{[2-(azetidin-1-yl)pyridin-4-yl]methyl}carbamate (V188225)

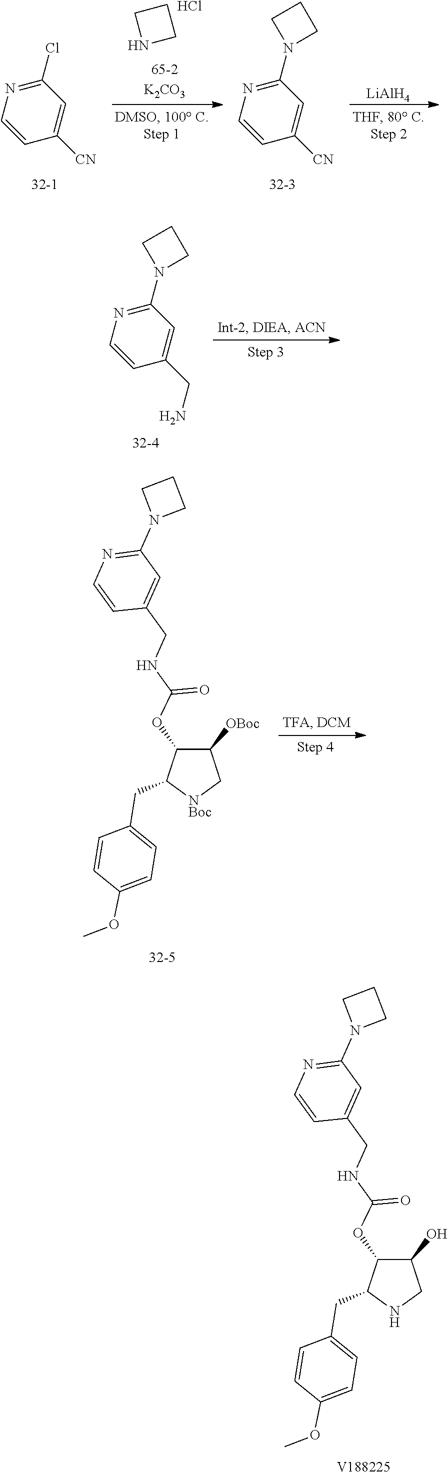

Step 1: To a stirred solution of 2-chloropyridine-4-carbonitrile hydrochloride (32-1, 500 mg, 2.857 mmol, 1 eq.) and azetidine (32-2, 244.6 mg, 4.28 mmol, 1.5 eq.) in DMSO (6 mL) was added K₂CO₃ (1184.5 mg, 8.57 mmol, 3 eq.) at 0° C. The resulting mixture was stirred at 100° C. for overnight. The crude product was purified by reversed phase flash to afford 2-(azetidin-1-yl)pyridine-4-carbonitrile (320 mg, 70.3%) as a light yellow solid. MS: m/z: Calc'd for C₉H₉N₃ [M+H]⁺160. Found, 160.

Step 2: To a stirred solution of 2-(azetidin-1-yl)pyridine-4-carbonitrile (32-3, 320 mg, 2.01 mmol, 1 eq.) in THF (5 mL) were added LiAlH₄ (152.5 mg, 4.02 mmol, 2 eq.) at 0° C. The resulting mixture was stirred at 80° C. for 2h. Upon completion, the reaction was quenched with Na₂SO₄·10H₂O. Filtered, washed with EA, the filtrate was concentrated, the crude product (300 mg) was purified by Prep-HPLC to afford 1-[2-(azetidin-1-yl)pyridin-4-yl]methanamine (71 mg, 21.6%) as a light yellow solid. MS: m/z: Calc'd for C₉H₁₃N₃[M+H]⁺164. Found, 164.

### Step 3 and 4

The title compound was prepared in 37.4% overall yield as a yellow oil according to Substitution reaction for synthesis of Carbamate; General Procedure I using 1-[2-(azetidin-1-yl)pyridin-4-yl]methanamine (32-4) in STEP 3; Boc Deprotection; General Procedure II in STEP 4. MS: m/z Calc'd for C₂₂H₂₈N₄O₄ [M+H]⁺413. Found, 413. ¹H NMR (400 MHz, Methanol-d₄) δ 7.84 (d, J=6.7 Hz, 1H), 7.24 (d, J=8.2 Hz, 2H), 6.92 (d, J=8.5 Hz, 2H), 6.85 (d, J=7.0 Hz, 1H), 6.70 (s, 1H), 5.00 (d, J=3.5 Hz, 1H), 4.47-4.38 (m, 3H), 4.35-4.30 (m, 4H), 4.20-4.18 (m, 1H), 3.80 (s, 3H), 3.62 (dd, J=12.6, 4.3 Hz, 1H), 3.24 (d, J=12.6 Hz, 1H), 3.14 (dd, J=14.2, 6.9 Hz, 1H), 3.01 (dd, J=14.1, 8.6 Hz, 1H), 2.59-2.57 (m, 2H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 23% B to 53% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-[2-(piperidin-4-yl)ethyl]carbamate (V188229)

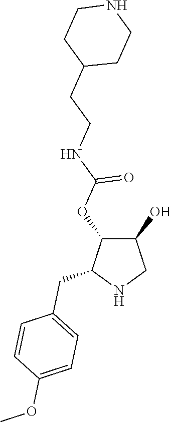 [SMILES: COc1ccc(C[C@H]2NC[C@H](O)[C@H]2OC(=O)NCCC2CCNCC2)cc1]

The title compound was prepared in 31.9% overall yield as a white semi-solid according to Substitution reaction for synthesis of Carbamate; General Procedure I using tert-butyl 4-(2-aminoethyl)piperidine-1-carboxylate in STEP 1; Boc Deprotection; General Procedure II in STEP 2. MS: m/z Calc'd for C₂₀H₃₁N₃O₄ [M+H]⁺378. Found, 378. ¹H NMR (400 MHz, Methanol-d₄) δ 7.27-7.20 (m, 2H), 6.96-6.89 (m, 2H), 4.94 (d, J=3.6 Hz, 1H), 4.38 (d, J=4.1 Hz, 1H), 4.19-4.13 (m, 1H), 3.80 (s, 3H), 3.60-3.56 (m, 1H), 3.42-3.38 (m, 2H), 3.28-3.18 (m, 3H), 3.11 (dd, J=14.1, 7.2 Hz, 1H), 3.04-2.91 (m, 3H), 2.01 (d, J=14.1 Hz, 2H), 1.74-1.68 (m, 1H), 1.59-1.53 (m, 2H), 1.50-1.36 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 9.57.

### Synthetic Procedure of (2R,3S,4S)-4-hydroxy-2-[(4-methoxyphenyl)methyl]pyrrolidin-3-yl N-(1H-indazol-7-ylmethyl)carbamate (V190459)

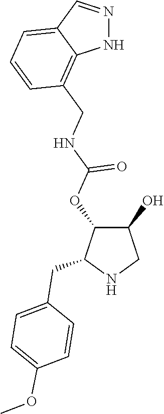 [SMILES: COc1ccc(C[C@H]2NC[C@H](O)[C@H]2OC(=O)NCc2cccc3cn[nH]c23)cc1]

The title compound was prepared in 31.6% overall yield as a white solid according to Substitution reaction for synthesis of Carbamate; General Procedure I using 1-(1H-indazol-7-yl)methanamine in STEP 1; Boc Deprotection; General Procedure II in STEP 2. MS: m/z C₂₁H₂₄N₄O₄[M+H]⁺397. Found 397. ¹H NMR (400 MHz, Methanol-d₄) δ 8.11 (d, J=7.9 Hz, 1H), 7.82-7.66 (m, 1H), 7.43-7.28 (m, 1H), 7.28-7.04 (m, 3H), 6.87-6.70 (m, 2H), 5.01-4.91 (m, 1H), 4.75-4.53 (m, 2H), 4.40 (dd, J=8.3, 4.3 Hz, 1H), 4.22-4.06 (m, 1H), 3.76 (d, J=8.1 Hz, 3H), 3.65-3.45 (m, 1H), 3.26-3.13 (m, 1H), 3.14-2.99 (m, 1H), 3.00-2.87 (m, 1H).

Prep-HPLC purification conditions: Column: Xselect CSH Prep C18 OBD Column, 30*150 mm, 5 μm; Mobile Phase A: Water (0.05% TFA), Mobile Phase B: ACN; Flow rate: 60 mL/min mL/min; Gradient: 6% B to 36% B in 7 min; Wave Length: 254 nm/220 nm; RT1(min): 6.8.

### Synthetic Procedure of (2R,3S,4S)-2-[(4-fluorophenyl)methyl]-4-hydroxypyrrolidin-3-yl N-{2-[(2S)-pyrrolidin-2-yl]ethyl}carbamate (V188406)

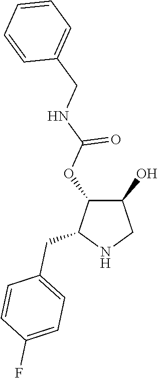 [SMILES: O=C(NCc1ccccc1)O[C@@H]1[C@@H](O)CN[C@@H]1Cc1ccc(F)cc1]

The title compound was prepared in 27.2% overall yield as a white solid according to Substitution reaction for synthesis of Carbamate; General Procedure I using tert-butyl (2R,3S,4S)-4-[(tert-butoxycarbonyl)oxy]-2-[(4-fluorophenyl)methyl]-3-[(4-nitrophenoxycarbonyl)oxy]pyrrolidine-1-carboxylate (Int-8) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2); benzylamine in STEP 1; Boc Deprotection; General Procedure II in STEP 2. MS: m/z Calc'd for C₁₉H₂₁FN₂O₃[M+H]⁺345. found 345. ¹H NMR (400 MHz, Methanol-d₄) δ 7.44-7.22 (m, 7H), 7.17-6.95 (m, 2H), 5.01-4.90 (m, 1H), 4.48-4.27 (m, 3H), 4.24-4.13 (m, 1H), 3.59 (dd, J=12.7, 4.3 Hz, 1H), 3.29-3.07 (m, 2H), 3.11-2.93 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 9% B to 39% B in 10 min; Wave Length: 254 nm/220 nm; RT1(min): 10.47.

### Synthetic Procedure of (2R,3S,4S)-2-[(4-fluorophenyl)methyl]-4-hydroxypyrrolidin-3-yl N-[(3-fluorophenyl)methyl]carbamate (V190506)

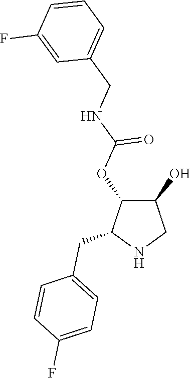 [SMILES: O=C(NCc1cccc(F)c1)O[C@@H]1[C@@H](O)CN[C@@H]1Cc1ccc(F)cc1]

The title compound was prepared in 11.3% overall yield as a white solid according to Addition reaction: Isocyanates and hydroxyl groups; General Procedure VI using 1-fluoro-3-(isocyanatomethyl)benzene in STEP 1; Boc Deprotection; General Procedure II in STEP 2. MS: m/z Calc'd for C₁₉H₂₀F₂N₂O₃[M+H]⁺363. found 363. ¹H NMR (400 MHz, Methanol-d₄) δ 7.48-7.26 (m, 3H), 7.23-6.97 (m, 5H), 4.96 (d, J=3.5 Hz, 1H), 4.41 (d, J=3.3 Hz, 1H), 4.39-4.27 (m, 2H), 4.26-4.12 (m, 1H), 3.59 (dd, J=12.6, 4.3 Hz, 1H), 3.24 (d, J=12.6 Hz, 1H), 3.21-3.13 (m, 1H), 3.10-3.00 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 11% B to 41% B in 10 min; Wave Length: 254 nm/220 nm; RT1(min): 8.87.

### Synthetic Procedure of (2R,3S,4S)-2-{[4-(dimethylamino)phenyl]methyl}-4-hydroxypyrrolidin-3-yl N-benzylcarbamate (V190512)

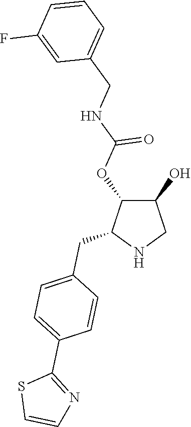 [SMILES: O=C(NCc1cccc(F)c1)O[C@@H]1[C@@H](O)CN[C@@H]1Cc1ccc(-c2nccs2)cc1]

The title compound was prepared in 6.12% overall yield as a white solid according to Stille Coupling with Pd(PPh₃)₄, CuI and CsF; General Procedure IV using 2-bromothiazole in STEP 1; Hydrolysis reaction with LiOH; General Procedure VII using tert-butyl (2S,3S,4S)-3-(acetyloxy)-4-[(tert-butoxycarbonyl)oxy]-2-{[4-(1,3-thiazol-2-yl)phenyl]methyl}pyrrolidine-1-carboxylate instead of tert-butyl (2R,3S,4S)-3-(acetyloxy)-4-[(tert-butoxycarbonyl)oxy]-2-[(4-fluorophenyl)methyl]pyrrolidine-1-carboxylate (3-1) in STEP 2; Addition reaction: Isocyanates and hydroxyl groups; General Procedure VI using tert-butyl (2S,3S,4S)-4-[(tert-butoxycarbonyl)oxy]-3-hydroxy-2-{[4-(1,3-thiazol-2-yl)phenyl]methyl}pyrrolidine-1-carboxylate instead of tert-butyl (2R,3S,4S)-4-[(tert-butoxycarbonyl)oxy]-2-[(4-fluorophenyl)methyl]-3-hydroxypyrrolidine-1-carboxylate (Int-7), 1-fluoro-3-(isocyanatomethyl)benzene in STEP 3; Boc Deprotection; General Procedure II in STEP 4. MS: m/z Calc'd for C₂₂H₂₂FN₃O₃S [M+H]⁺428. found 428. ¹H NMR (400 MHz, Methanol-d₄) δ 7.97-7.87 (m, 3H), 7.64 (d, J=3.3 Hz, 1H), 7.53-7.30 (m, 3H), 7.30-6.87 (m, 3H), 5.00 (d, J=3.6 Hz, 1H), 4.48-4.13 (m, 4H), 3.62 (dd, J=12.9, 4.2 Hz, 1H), 3.30-3.20 (m, 2H), 3.13 (dd, J=14.2, 8.2 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 10% B to 40% B in 10 min; Wave Length: 254 nm/220 nm; RT1(min): 8.9.

### Synthetic Procedure of (2R,3S,4S)-4-hydroxy-2-{[4-(1,3-oxazol-2-yl)phenyl]methyl}pyrrolidin-3-yl N-[(3-fluorophenyl)methyl]carbamate (V190514)

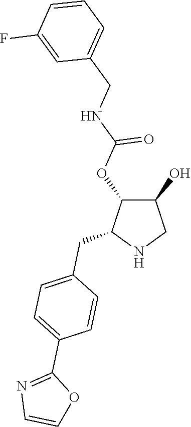 [SMILES: O=C(NCc1cccc(F)c1)O[C@@H]1[C@@H](O)CN[C@@H]1Cc1ccc(-c2ncco2)cc1]

The title compound was prepared in 7.51% overall yield as a white solid according to Stille Coupling with Pd(PPh₃)₄, CuI and CsF; General Procedure IV using tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (Int-3) instead of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (Int-6), 2-(tributylstannyl)-1,3-oxazole in STEP 1; Hydrolysis reaction with LiOH; General Procedure V using tert-butyl (2R,3S,4S)-3-(acetyloxy)-4-[(tert-butoxycarbonyl)oxy]-2-{[4-(1,3-oxazol-2-yl)phenyl]methyl}pyrrolidine-1-carboxylate instead of tert-butyl (2R,3S,4S)-3-(acetyloxy)-4-[(tert-butoxycarbonyl)oxy]-2-[(4-fluorophenyl)methyl]pyrrolidine-1-carboxylate (3-1) in STEP 2; Addition reaction: Isocyanates and hydroxyl groups; General Procedure VI using tert-butyl (2R,3S,4S)-4-[(tert-butoxycarbonyl)oxy]-3-hydroxy-2-{[4-(1,3-oxazol-2-yl)phenyl]methyl}pyrrolidine-1-carboxylate instead of tert-butyl (2R,3S,4S)-4-[(tert-butoxycarbonyl)oxy]-2-[(4-fluorophenyl)methyl]-3-hydroxypyrrolidine-1-carboxylate (Int-7), 1-fluoro-3-(isocyanatomethyl)benzene in STEP 3; Boc Deprotection; General Procedure II in STEP 4. MS: m/z Calc'd for C₂₂H₂₂FN₃O₄[M+H]⁺412. found 412. ¹H NMR (400 MHz, Methanol-d₄) δ 8.04-7.98 (m, 3H), 7.47 (d, J=8.0 Hz, 2H), 7.41-7.36 (m, 1H), 7.35-7.28 (m, 1H), 7.16 (d, J=7.6 Hz, 1H), 7.12-7.06 (m, 1H), 7.05-7.01 (m, 1H), 5.00 (s, 1H), 4.50-4.20 (m, 4H), 3.60 (dd, J=12.5, 4.2 Hz, 1H), 3.31-3.21 (m, 2H), 3.13 (dd, J=13.9, 8.4 Hz, 1H).

Prep-HPLC purification conditions: Column: Xselect CSH Prep C18 Column, 30*150 mm, 5 μm; Mobile Phase A: Water (0.1% FA), Mobile Phase B: ACN; Flow rate: 60 m/min; Gradient: 8% B to 38% B in 10 min; Wave Length: 254 nm/220 nm; RT1(min): 10.25.

### Synthetic Procedure of (2R,3S,4S)-4-hydroxy-2-{[4-(1,2-oxazol-4-yl)phenyl]methyl}pyrrolidin-3-yl N-[(3-fluorophenyl)methyl]carbamate (V190543)

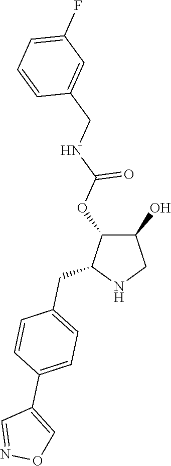 [SMILES: O=C(NCc1cccc(F)c1)O[C@@H]1[C@@H](O)CN[C@@H]1Cc1ccc(-c2cnoc2)cc1]

The title compound was prepared in 5.62% overall yield as a white solid according to Stille Coupling with Pd(PPh₃)₄, CuI and CsF; General Procedure IV using 4-iodo-1,2-oxazole in STEP 1; Hydrolysis reaction with LiOH; General Procedure V using tert-butyl (2R,3S,4S)-3-(acetyloxy)-4-[(tert-butoxycarbonyl)oxy]-2-{[4-(1,2-oxazol-4-yl)phenyl]methyl}pyrrolidine-1-carboxylate instead of tert-butyl (2R,3S,4S)-3-(acetyloxy)-4-[(tert-butoxycarbonyl)oxy]-2-[(4-fluorophenyl)methyl]pyrrolidine-1-carboxylate (3-1) in STEP 2; Addition reaction: Isocyanates and hydroxyl groups; General Procedure VI using tert-butyl (2R,3S,4S)-4-[(tert-butoxycarbonyl)oxy]-3-hydroxy-2-{[4-(1,2-oxazol-4-yl)phenyl]methyl}pyrrolidine-1-carboxylate instead of tert-butyl (2R,3S,4S)-4-[(tert-butoxycarbonyl)oxy]-2-[(4-fluorophenyl)methyl]-3-hydroxypyrrolidine-1-carboxylate (Int-7), 1-fluoro-3-(isocyanatomethyl)benzene in STEP 3; Boc Deprotection; General Procedure II in STEP 4. MS: m/z Calc'd for C₂₂H₂₂FN₃O₄[M+H]⁺412. found 412. ¹H NMR (400 MHz, Methanol-d₄) δ 9.06 (s, 1H), 8.82 (s, 1H), 7.61 (d, J=8.1 Hz, 2H), 7.38 (dd, J=6.8, 4.1 Hz, 3H), 7.24-6.99 (m, 3H), 4.97 (d, J=3.5 Hz, 1H), 4.42 (s, 1H), 4.39-4.28 (m, 2H), 4.28-4.18 (m, 1H), 3.68-3.56 (m, 1H), 3.29-3.27 (m, 1H), 3.24-3.16 (m, 1H), 3.09 (dd, J=14.0, 8.1 Hz, 1H).

Prep-HPLC purification conditions: Column: XBridge Prep Shield RP18 OBD C18 Column, 30*150 mm, 5 μm; Mobile Phase A: Water (0.05% TFA), Mobile Phase B: ACN; Flow rate: 60 mL/min; Gradient: 14% B to 35% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-[(3-fluorophenyl)methyl]carbamate (V190545)

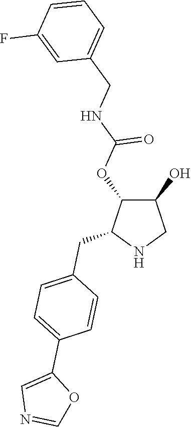 [SMILES: O=C(NCc1cccc(F)c1)O[C@@H]1[C@@H](O)CN[C@@H]1Cc1ccc(-c2cnco2)cc1]

The title compound was prepared in 12.3% overall yield as a white solid according to Addition reaction: Isocyanates and hydroxyl groups; General Procedure VI using tert-butyl (2S,3S,4S)-4-[(tert-butoxycarbonyl)oxy]-3-({[(3-fluorophenyl)methyl]carbamoyl}oxy)-2-{[4-(1,3-oxazol-5-yl)phenyl]methyl}pyrrolidine-1-carboxylate (Int-9) instead of tert-butyl (2R,3S,4S)-4-[(tert-butoxycarbonyl)oxy]-2-[(4-fluorophenyl)methyl]-3-hydroxypyrrolidine-1-carboxylate (Int-7), 1-fluoro-3-(isocyanatomethyl)benzene in STEP 1; Boc Deprotection; General Procedure II in STEP 2. MS: m/z Calc'd for C₂₂H₂₂FN₃O₄[M+H]⁺412. found 412. ¹H NMR (400 MHz, Methanol-d₄) δ 8.28 (s, 1H), 7.70 (d, J=8.1 Hz, 2H), 7.53 (s, 1H), 7.48-7.31 (m, 3H), 7.27-7.14 (m, 1H), 7.13-7.08 (m, 1H), 7.07-6.97 (m, 1H), 4.96 (d, J=3.5 Hz, 1H), 4.64-4.10 (m, 4H), 3.78-3.54 (m, 1H), 3.26-3.23 (m, 1H), 3.23-3.17 (m, 1H), 3.11-3.09 (m, 1H).

Prep-HPLC purification conditions: Column: Xselect CSH Prep Fluoro-Phenyl C18 Column, 30*150 mm, 5 μm; Mobile Phase A: Water (0.05% TFA), Mobile Phase B: ACN; Flow rate: 60 mL/min; Gradient: 5% B to 35% B in 10 min; Wave Length: 254 nm/220 nm; RT1(min): 7.9.

### Synthetic Procedure of (2R,3S,4S)-2-[(4-fluorophenyl)methyl]-4-hydroxypyrrolidin-3-yl N-{2-[(2S)-pyrrolidin-2-yl]ethyl}carbamate (V191886)

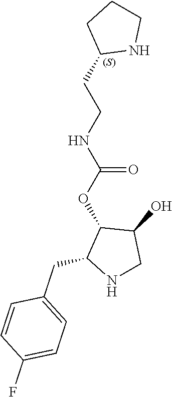

The title compound was prepared in 27.2% overall yield as a white solid according to Substitution reaction for synthesis of Carbamate; General Procedure I using tert-butyl (2R,3S,4S)-4-[(tert-butoxycarbonyl)oxy]-2-[(4-fluorophenyl)methyl]-3-[(4-nitrophenoxycarbonyl)oxy]pyrrolidine-1-carboxylate (Int-8) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate in STEP 1; Boc Deprotection; General Procedure II in STEP 2. MS: m/z Calc'd for C₁₈H₂₆FN₃O₃[M+H]⁺352. found 352. ¹H NMR (400 MHz, Methanol-d₄) δ 7.40-7.31 (m, 2H), 7.16-7.03 (m, 2H), 4.98 (d, J=3.5 Hz, 1H), 4.39 (d, J=4.1 Hz, 1H), 4.26-4.16 (m, 1H), 3.67-3.49 (m, 2H), 3.32-3.26 (m, 3H), 3.26-3.13 (m, 3H), 3.12-2.98 (m, 1H), 2.36-2.25 (m, 1H), 2.19-1.86 (m, 4H), 1.78-1.65 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 8.89.

### Synthetic Procedure of (2R,3S,4S)-4-hydroxy-2-[(4-methoxyphenyl)methyl]pyrrolidin-3-yl N-[(4-methanesulfonamidophenyl)methyl]carbamate (V191955)

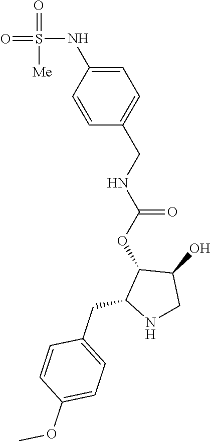 [SMILES: COc1ccc(C[C@H]2NC[C@H](O)[C@H]2OC(=O)NCc2ccc(NS(C)(=O)=O)cc2)cc1]

The title compound was prepared in 37.7% overall yield as a white solid according to Substitution reaction for synthesis of Carbamate; General Procedure I using N-[4-(aminomethyl)phenyl]methanesulfonamide in STEP 1; Boc Deprotection; General Procedure II in STEP 2. MS: m/z Calc'd for C₂₁H₂₇N₃O₆S [M+H]⁺450. found 450. ¹H NMR (400 MHz, Methanol-d₄) δ 7.33 (d, J=8.3 Hz, 2H), 7.26 (d, J=8.4 Hz, 2H), 7.19 (d, J=8.4 Hz, 2H), 6.92-6.83 (m, 2H), 4.93 (d, J=3.6 Hz, 1H), 4.39 (d, J=4.3 Hz, 1H), 4.36-4.22 (m, 2H), 4.19-4.13 (m, 1H), 3.79 (s, 3H), 3.57 (dd, J=12.6, 4.3 Hz, 1H), 3.22 (d, J=12.6 Hz, 1H), 3.10 (dd, J=13.9, 7.6 Hz, 1H), 3.01-2.91 (m, 1H), 2.95 (s, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 3% B to 33% B in 10 min; Wave Length: 254 nm/220 nm; RT1(min): 10.28.

### Synthetic Procedure of (2R,3S,4S)-2-({3′,4′-difluoro-[1,1′-biphenyl]-4-yl}methyl)-4-hydroxypyrrolidin-3-yl N-{2-[(2S)-pyrrolidin-2-yl]ethyl}carbamate (V191962)

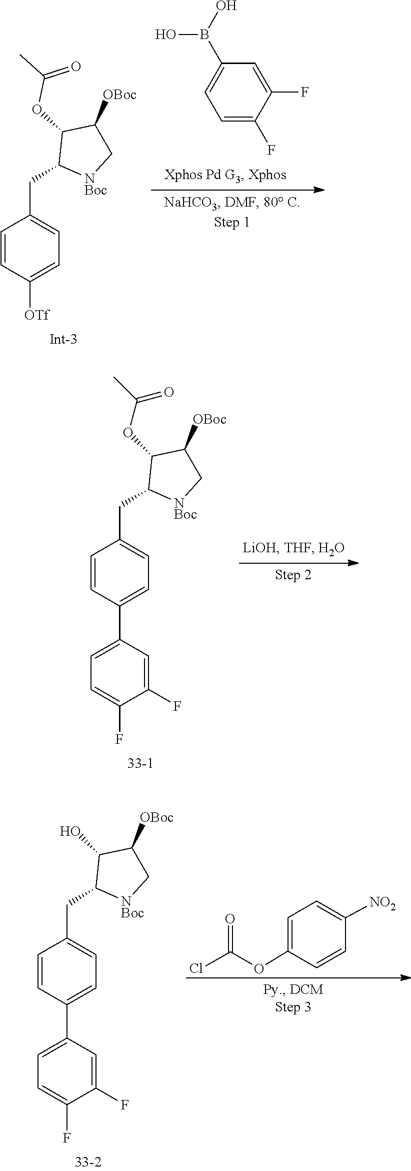

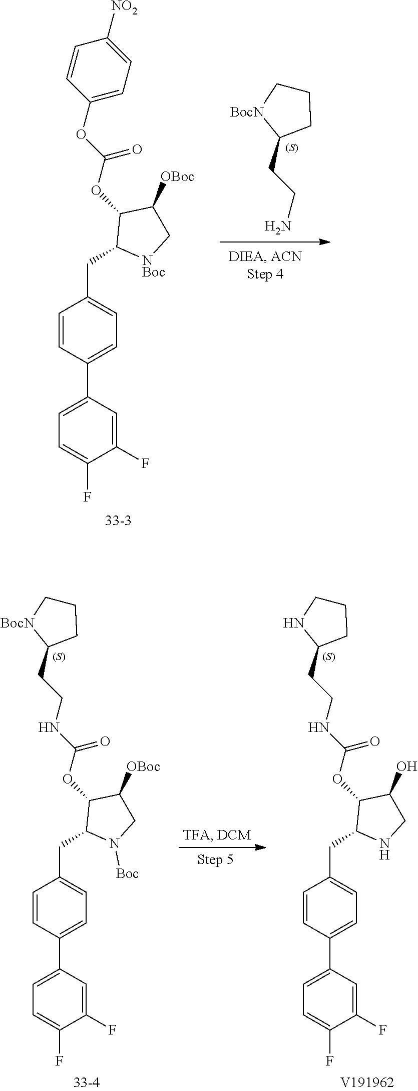

Step 1: To a degassed mixture of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (150 mg, 0.25 mmol, 1 equiv) and 3,4-difluorophenylboronic acid (60.8 mg, 0.38 mmol, 1.5 equiv) in DMF (3 mL) was added NaHCO₃ (64.7 mg, 0.77 mmol, 3 equiv) followed by XPhos Pd G3 (21.7 mg, 0.02 mmol, 0.1 equiv) and XPhos (24.5 mg, 0.05 mmol, 0.2 equiv) at room temperature. The reaction was placed under vacuum, sonicated and backfilled with nitrogen. The reaction mixture was stirred at 80° C. for overnight. After completion of the reaction, the reaction mixture was quenched with water 30 mL. The aqueous layer was extracted with ethyl acetate (100 mL). Then, the combined organic phase was washed with brine, dried over anhydrous Na₂SO₄. Purified by column chromatography on silica gel (Combiflash) (PE/EtOAc=4:1) to afford tert-butyl (2S,3S,4S)-3-(acetyloxy)-4-[(tert-butoxycarbonyl)oxy]-2-({3′,4′-difluoro-[1,1′-biphenyl]-4-yl}methyl)pyrrolidine-1-carboxylate (120 mg, 85.2%) as a light yellow oil. MS: m/z: Calc'd for C₂₉H₃₅F₂NO₇ [M−56−100]⁺ 392, found 392.

Step 2: The compound was prepared in 90.2% yield as a light yellow oil according to Hydrolysis reaction with LiOH; General Procedure V using tert-butyl (2S,3S,4S)-3-(acetyloxy)-4-[(tert-butoxycarbonyl)oxy]-2-({3′,4′-difluoro-[1,1′-biphenyl]-4-yl}methyl)pyrrolidine-1-carboxylate (33-1) instead of tert-butyl (2R,3S,4S)-3-(acetyloxy)-4-[(tert-butoxycarbonyl)oxy]-2-[(4-fluorophenyl)methyl]pyrrolidine-1-carboxylate (3-1) in STEP 2. MS: m/z Calc'd for C₂₇H₃₃F₂NO₆ [M−56−100]⁺350. found 350.

Step 3: To a stirred solution of tert-butyl (2S,3S,4S)-4-[(tert-butoxycarbonyl)oxy]-2-({3′,4′-difluoro-[1,1′-biphenyl]-4-yl}methyl)-3-hydroxypyrrolidine-1-carboxylate (33-2, 100 mg, 0.19 mmol, 1 equiv) in DCM (3 mL) was added 4-nitrophenyl carbonochloridate (59.8 mg, 0.29 mmol, 1.5 equiv) and Pyridine (46.9 mg, 0.59 mmol, 3 equiv) at room temperature under nitrogen atmosphere. The resulting mixture was stirred for 2 h at room temperature. Desired product could be detected by LCMS. The resulting mixture was concentrated under vacuum. The residue was purified by reverse phase flash with the following conditions (5.0 mmol/L NH₄HCO₃, ACN) to afford tert-butyl (2S,3S,4S)-4-[(tert-butoxycarbonyl)oxy]-2-({3′,4′-difluoro-[1,1′-biphenyl]-4-yl}methyl)-3-[(4-nitrophenoxycarbonyl)oxy]pyrrolidine-1-carboxylate (100 mg, 75.38%) as a light yellow oil. MS: m/z: Calc'd for C₃₄H₃₆F₂N₂O₁₀ [M−100]⁺ 571. found 571.

Step 4 and 5: The title compound was prepared in 33.1% overall yield as an off-white solid according to Substitution reaction for synthesis of Carbamate; General Procedure I using tert-butyl (2S)-2-(2-aminoethyl)pyrrolidine-1-carboxylate in STEP 4; Boc Deprotection; General Procedure II in STEP 5. MS: m/z Calc'd for C₂₄H₂₉F₂N₃O₃[M+H]⁺446. found 446. ¹H NMR (400 MHz, Methanol-d₄) δ 7.67-7.60 (m, 2H), 7.58-7.52 (m, 1H), 7.49-7.40 (m, 3H), 7.35-7.32 (m, 1H), 5.04-4.98 (m, 1H), 4.41 (d, J=4.2 Hz, 1H), 4.28-4.24 (m, 1H), 3.65-3.52 (m, 2H), 3.35 (d, J=7.9 Hz, 1H), 3.30-3.19 (m, 5H), 3.11-3.09 (m, 1H), 2.42-2.22 (m, 1H), 2.16-2.07 (m, 1H), 2.08-1.87 (m, 3H), 1.79-1.65 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 4% B to 34% B in 10 min; Wave Length: 254 nm/220 nm; RT1(min): 9.22.

### Synthetic Procedure of (2R,3S,4S)-2-({3′,4′-difluoro-[1,1′-biphenyl]-4-yl}methyl)-4-hydroxypyrrolidin-3-yl N-{2-[(2R)-pyrrolidin-2-yl]ethyl}carbamate (V192037)

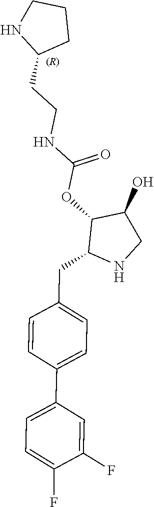

The title compound was prepared in 35.1% overall yield as an off-white solid according to Substitution reaction for synthesis of Carbamate; General Procedure I using tert-butyl (2R,3S,4S)-4-[(tert-butoxycarbonyl)oxy]-2-({3′,4′-difluoro-[1,1′-biphenyl]-4-yl}methyl)-3-[(4-nitrophenoxycarbonyl)oxy]pyrrolidine-1-carboxylate (33-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2R)-2-(2-aminoethyl)pyrrolidine-1-carboxylate in STEP 1; Boc Deprotection; General Procedure II in STEP 2. MS: m/z Calc'd for C₂₄H₂₉F₂N₃O₃[M+H]⁺446, found 446. ¹H NMR (400 MHz, Methanol-d₄) δ 7.66-7.59 (m, 2H), 7.56-7.52 (m, 1H), 7.48-7.40 (m, 3H), 7.35-7.32 (m, 1H), 5.04-4.98 (m, 1H), 4.41 (d, J=4.2 Hz, 1H), 4.28-4.18 (m, 1H), 3.66-3.53 (m, 2H), 3.35 (d, J=4.1 Hz, 2H), 3.30 (d, J=7.1 Hz, 2H), 3.23 (dd, J=13.5, 7.9 Hz, 2H), 3.11 (dd, J=14.1, 8.5 Hz, 1H), 2.35-2.27 (m, 1H), 2.18-1.91 (m, 4H), 1.79-1.62 (m, 1H).

Prep-HPLC purification conditions: Column: Welch Ultimate PFP-C18 30*250, 10 μm; Mobile Phase A: Water (0.05% TFA), Mobile Phase B: ACN; Flow rate: 60 mL/min; Gradient: 3% B to 33% B in 10 min; Wave Length: 254 nm/220 nm; RT1(min): 10.48.

### Synthetic Procedure of (2R,3S,4S)-4-hydroxy-2-{[4-(1,3-oxazol-5-yl)phenyl]methyl}pyrrolidin-3-yl N-{2-[(2S)-pyrrolidin-2-yl]ethyl}carbamate (V192047)

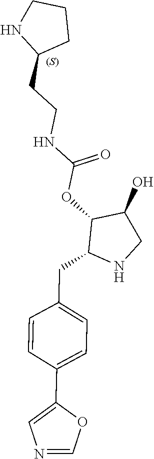

The title compound was prepared in 23.3% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate in STEP 1; Boc Deprotection; General Procedure II in STEP 2. MS: m/z Calc'd for C₂₁H₂₈N₄O₄ [M+H]⁺401. found 401. ¹H NMR (400 MHz, Methanol-d₄) δ 8.23 (s, 1H), 7.70-7.64 (m, 2H), 7.48 (s, 1H), 7.38-7.32 (m, 2H), 4.67 (d, J=3.9 Hz, 1H), 4.18-4.12 (m, 1H), 3.64-3.56 (m, 1H), 3.30-3.28 (m, 1H), 3.28-3.17 (m, 2H), 3.11-2.97 (m, 2H), 2.97-2.83 (m, 3H), 2.74 (dd, J=12.1, 2.6 Hz, 1H), 2.09-1.97 (m, 1H), 1.94-1.81 (m, 2H), 1.79-1.68 (m, 2H), 1.48-1.35 (m, 1H).

Prep-HPLC purification conditions: Column: XBridge Prep OBD C18 Column, 30*150 mm, 5 μm; Mobile Phase A: Water (10 nmol/LNH₄HCO₃), Mobile Phase B: ACN; Flow rate: 60 mL/min; Gradient: 2% B to 22% B in 10 min; Wave Length: 254 nm/220 nm; RT1(min): 8.6.

### Synthetic Procedure of (2R,3S,4S)-4-hydroxy-2-{[4-(1,3-oxazol-5-yl)phenyl]methyl}pyrrolidin-3-yl N-{2-[(2R)-pyrrolidin-2-yl]ethyl}carbamate (V192048)

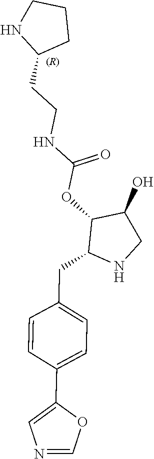

The title compound was prepared in 22.3% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2R)-2-(2-aminoethyl)pyrrolidine-1-carboxylate in STEP 1; Boc Deprotection; General Procedure II in STEP 2. MS: m/z Calc'd for C₂₁H₂₈N₄O₄ [M+H]⁺401. found 401. ¹H NMR (400 MHz, Methanol-d₄) δ 8.23 (s, 1H), 7.67 (d, J=8.1 Hz, 2H), 7.48 (s, 1H), 7.34 (d, J=8.2 Hz, 2H), 4.68 (d, J=4.8 Hz, 1H), 4.15 (d, J=4.8 Hz, 1H), 3.77-3.55 (m, 1H), 3.29 (s, 1H), 3.24 (t, J=7.0 Hz, 2H), 3.19-3.12 (m, 1H), 3.12-3.03 (m, 1H), 2.99-2.81 (m, 3H), 2.74-2.72 (m, 1H), 2.12-2.02 (m, 1H), 2.00-1.80 (m, 2H), 1.80-1.68 (m, 2H), 1.51-1.38 (m, 1H).

Prep-HPLC purification conditions: Column: XBridge Prep OBD C18 Column, 30*150 mm, 5 μm; Mobile Phase A: Water (10 nmol/LNH₄HCO₃), Mobile Phase B: ACN; Flow rate: 60 mL/min; Gradient: 2% B to 23% B in 10 min; Wave Length: 254 nm/220 nm; RT1(min): 8.52.

### Synthetic Procedure of (2R,3S,4S)-2-({3′,4′-difluoro-[1,1′-biphenyl]-4-yl}methyl)-4-hydroxypyrrolidin-3-yl N-[2-(piperidin-4-yl)ethyl]carbamate (V192051)

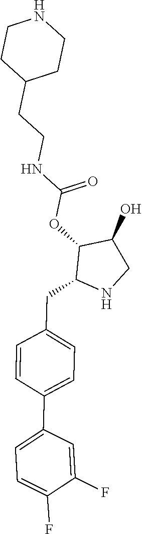 [SMILES: O=C(NCCC1CCNCC1)O[C@@H]1[C@@H](O)CN[C@@H]1Cc1ccc(-c2ccc(F)c(F)c2)cc1]

The title compound was prepared in 42.5% overall yield as a white solid according to Substitution reaction for synthesis of Carbamate; General Procedure I using tert-butyl (2R,3S,4S)-4-[(tert-butoxycarbonyl)oxy]-2-({3′,4′-difluoro-[1,1′-biphenyl]-4-yl}methyl)-3-[(4-nitrophenoxycarbonyl)oxy]pyrrolidine-1-carboxylate (33-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 4-(2-aminoethyl)piperidine-1-carboxylate in STEP 1; Boc Deprotection; General Procedure II in STEP 2. MS: m/z Calc'd for C₂₅H₃₁F₂N₃O₃[M+H]⁺460, found 460. ¹H NMR (400 MHz, Methanol-d₄) δ 7.64-7.58 (m, 2H), 7.56-7.50 (m, 1H), 7.42 (d, J=8.0 Hz, 3H), 7.35-7.28 (m, 1H), 4.98 (d, J=3.4 Hz, 1H), 4.39 (d, J=4.3 Hz, 1H), 4.26-4.20 (m, 1H), 3.69-3.61 (m, 1H), 3.44-3.35 (m, 2H), 3.28-3.18 (m, 4H), 3.18-3.08 (m, 1H), 3.02-2.89 (m, 2H), 2.00 (d, J=14.1 Hz, 2H), 1.78-1.66 (m, 1H), 1.57-1.55 (m, 2H), 1.51-1.36 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 9% B to 39% B in 10 min; Wave Length: 254 nm/220 nm; RT1(min): 8.5.

### Synthetic Procedure of (2R,3S,4S)-2-({3′,4′-difluoro-[1,1′-biphenyl]-4-yl}methyl)-4-hydroxypyrrolidin-3-ylN-(1,2,3,4-tetrahydroisoquinolin-6-ylmethyl)carbamate (V192057)

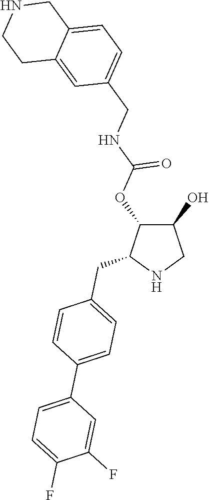 [SMILES: O=C(NCc1ccc2c(c1)CCNC2)O[C@@H]1[C@@H](O)CN[C@@H]1Cc1ccc(-c2ccc(F)c(F)c2)cc1]

The title compound was prepared in 18.9% overall yield as a white solid according to Substitution reaction for synthesis of Carbamate; General Procedure I using tert-butyl (2R,3S,4S)-4-[(tert-butoxycarbonyl)oxy]-2-({3′,4′-difluoro-[1,1′-biphenyl]-4-yl}methyl)-3-[(4-nitrophenoxycarbonyl)oxy]pyrrolidine-1-carboxylate (33-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 6-(aminomethyl)-3,4-dihydro-1H-isoquinoline-2-carboxylate in STEP 1; Boc Deprotection; General Procedure II in STEP 2. MS: m/z Calc'd for C₂₁H₂₈N₄O₄ [M+H]⁺401, found 401. ¹H NMR (400 MHz, Methanol-d₄) δ 7.60 (d, J=8.1 Hz, 2H), 7.57-7.52 (m, 1H), 7.44-7.30 (m, 4H), 7.30-7.19 (m, 3H), 5.01 (d, J=3.5 Hz, 1H), 4.43-4.37 (m, 1H), 4.36-4.31 (m, 3H), 4.27 (d, J=12.8 Hz, 1H), 3.61 (dd, J=12.7, 4.2 Hz, 1H), 3.49 (t, J=6.4 Hz, 2H), 3.34 (s, 1H), 3.29-3.18 (m, 2H), 3.16-3.05 (m, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 10% B to 40% B in 15 min; Wave Length: 254 nm/220 nm; RT1(min): 16.5.

### Synthetic Procedure of (2R,3S,4S)-4-hydroxy-2-({4-[2-(trifluoromethyl)-1,3-thiazol-5-yl]phenyl}methyl)pyrrolidin-3-yl N-[(3-fluorophenyl)methyl]carbamate (V192114)

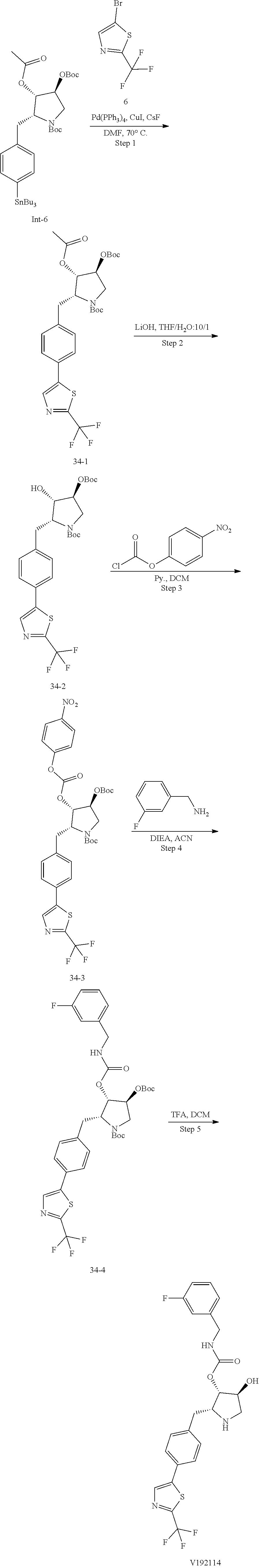

### Step 1 and 2

The compound was prepared in 38.3% overall yield as a yellow solid according to Stille Coupling with Pd(PPh₃)₄, CuI and CsF; General Procedure IV using 5-bromo-2-(trifluoromethyl)-1,3-thiazole in STEP 1; Hydrolysis reaction with LiOH; General Procedure V using tert-butyl (2R,3S,4S)-3-(acetyloxy)-4-[(tert-butoxycarbonyl)oxy]-2-({4-[2-(trifluoromethyl)-1,3-thiazol-5-yl]phenyl}methyl)pyrrolidine-1-carboxylate (34-1) instead of tert-butyl (2R,3S,4S)-3-(acetyloxy)-4-[(tert-butoxycarbonyl)oxy]-2-[(4-fluorophenyl)methyl]pyrrolidine-1-carboxylate (3-1) in STEP 2. MS: m/z Calc'd for C₂₅H₃₁F₃N₂O₆S [M+H]⁺545. found 545.

Step 3: To a stirred solution of tert-butyl (2R,3S,4S)-4-[(tert-butoxycarbonyl)oxy]-3-hydroxy-2-({4-[2-(trifluoromethyl)-1,3-thiazol-5-yl]phenyl}methyl)pyrrolidine-1-carboxylate (34-2, 85 mg, 0.15 mmol, 1 equiv) in DCM (5 mL) was added 4-nitrophenyl carbonochloridate (62.9 mg, 0.31 mmol, 2 equiv) and Pyridine (37.0 mg, 0.46 mmol, 3 equiv) at room temperature under nitrogen atmosphere. The resulting mixture was stirred for 2 h at room temperature. Desired product could be detected by LCMS. The resulting mixture was concentrated under vacuum. The residue was purified by Prep-TLC (PE/EA 3:1) to afford tert-butyl (2R,3S,4S)-4-[(tert-butoxycarbonyl)oxy]-3-[(4-nitrophenoxycarbonyl)oxy]-2-({4-[2-(trifluoromethyl)-1,3-thiazol-5-yl]phenyl}methyl)pyrrolidine-1-carboxylate (100 mg, 90.2%) as a light yellow solid. MS: m/z: Calc'd for C₃₂H₃₄F₃N₃O₁₀S [M+H]⁺710. found 710.

### Step 4 and 5

The title compound was prepared in 32.8% overall yield as a white solid according to Substitution reaction for synthesis of Carbamate; General Procedure I using 1-(3-fluorophenyl)methanamine in STEP 4; Boc Deprotection; General Procedure II in STEP 5. MS: m/z Calc'd for C₂₃H₂₁F₄N₃O₃[M+H]⁺496. found 496. ¹H NMR (400 MHz, Methanol-d₄) δ 8.31-8.26 (m, 1H), 7.73-7.67 (m, 2H), 7.47-7.32 (m, 3H), 7.17 (d, J=7.7 Hz, 1H), 7.13-7.06 (m, 1H), 7.06-6.98 (m, 1H), 4.97 (d, J=3.6 Hz, 1H), 4.55-4.17 (m, 4H), 3.61-3.59 (m, 1H), 3.31-3.18 (m, 2H), 3.13-3.11 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 25% B to 55% B in 13 min; Wave Length: 254 nm/220 nm; RT1(min): 12.6.

### Synthetic Procedure of (2R,3S,4S)-4-hydroxy-2-{[4-(1,3-oxazol-5-yl)phenyl]methyl}pyrrolidin-3-yl N-{2-[(2S)-pyrrolidin-2-yl]ethyl}carbamate (V192136)

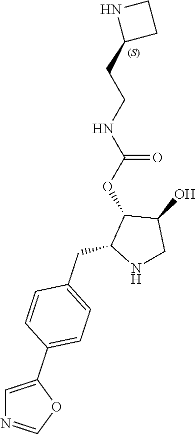

The title compound was prepared in 24.6% overall yield as an off-white semi-solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2R)-2-(2-aminoethyl)azetidine-1-carboxylate in STEP 1; Boc Deprotection; General Procedure II in STEP 2. MS: m/z Calc'd for C₂₀H₂₆N₄O₄ [M+H]⁺387. found 387. ¹H NMR (400 MHz, Methanol-d₄) δ 8.56 (s, 1H), 8.27 (s, 1H), 8.17-7.94 (m, OH), 7.79-7.72 (m, 2H), 7.54 (s, 1H), 7.44 (d, J=8.0 Hz, 2H), 4.95 (d, J=3.5 Hz, 1H), 4.52 (t, J=7.7 Hz, 1H), 4.37 (s, 1H), 4.18 (s, 1H), 4.13-4.01 (m, 1H), 3.95-3.85 (m, 1H), 3.61-3.53 (m, 1H), 3.29-3.15 (m, 4H), 3.16-3.00 (m, 1H), 2.65-2.54 (m, 1H), 2.48-2.29 (m, 1H), 2.24-2.02 (m, 2H).

Prep-HPLC purification conditions: Column: Welch Ultimate LP-C18 30*150, 10 μm; Mobile Phase A: Water (0.1% FA), Mobile Phase B: ACN; Flow rate: 60 mL/min mL/min; Gradient: 33% B to 63% B in 10 min; Wave Length: 254 nm/220 nm; RT1(min): 8.27.

### Synthetic Procedure of (2R,3S,4S)-4-hydroxy-2-{[4-(1,3-oxazol-5-yl)phenyl]methyl}pyrrolidin-3-yl N-{2-[(2S)-pyrrolidin-2-yl]ethyl}carbamate (V192232)

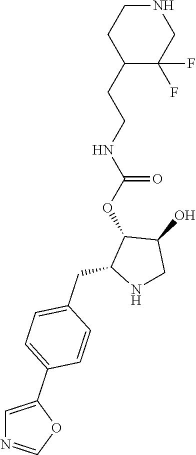 [SMILES: O=C(NCCC1CCNCC1(F)F)O[C@@H]1[C@@H](O)CN[C@@H]1Cc1ccc(-c2cnco2)cc1]

The title compound was prepared in 31.2% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 4-(2-aminoethyl)-3,3-difluoropiperidine-1-carboxylate in STEP 1; Boc Deprotection; General Procedure II in STEP 2. MS: m/z Calc'd for C₂₂H₂₈F₂N₄O₄[M+H]⁺451, found 451. ¹H NMR (400 MHz, Methanol-d₄) δ 8.27 (s, 1H), 7.75 (dd, J=8.4, 2.1 Hz, 2H), 7.53 (s, 1H), 7.44 (dd, J=8.2, 3.4 Hz, 2H), 4.98 (d, J=3.5 Hz, 1H), 4.40 (d, J=4.1 Hz, 1H), 4.27 (d, J=9.3 Hz, 1H), 3.72 (s, 1H), 3.65-3.56 (m, 1H), 3.55-3.37 (m, 3H), 3.32-3.18 (m, 3H), 3.14 (s, 2H), 2.25 (d, J=15.9 Hz, 2H), 2.07 (d, J=12.3 Hz, 1H), 1.74 (d, J=12.7 Hz, 1H), 1.65-1.39 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 15% B in 16 min; Wave Length: 254 nm/220 nm; RT1(min): 15.5.

### Synthetic Procedure of (2R,3S,4S)-4-hydroxy-2-{[4-(1,3-oxazol-5-yl)phenyl]methyl}pyrrolidin-3-yl N-(cyclopropylmethyl)carbamate (V192262)

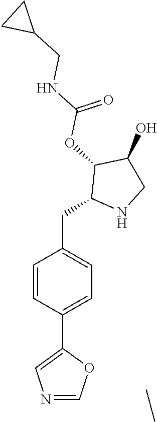

The title compound was prepared in 29.7% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-cyclopropylmethanamine in STEP 1; Boc Deprotection; General Procedure II in STEP 2. MS: m/z Calc'd for C₁₉H₂₃N₃O₄ [M+H]⁺358. found 358. ¹H NMR (400 MHz, Methanol-d₄) δ 8.27 (s, 1H), 7.79-7.72 (m, 2H), 7.54 (s, 1H), 7.44 (d, J=8.2 Hz, 2H), 4.98 (s, 1H), 4.43-4.37 (m, 1H), 4.25 (t, J=5.8 Hz, 1H), 3.60-3.58 (m, 1H), 3.24-3.22 (m, 2H), 3.16-2.92 (m, 3H), 1.18-0.94 (m, 1H), 0.59-0.50 (m, 2H), 0.39-0.15 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 7% B to 37% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-[2-(3-cyano-3-hydroxyazetidin-1-yl)ethyl]carbamate (V187567)

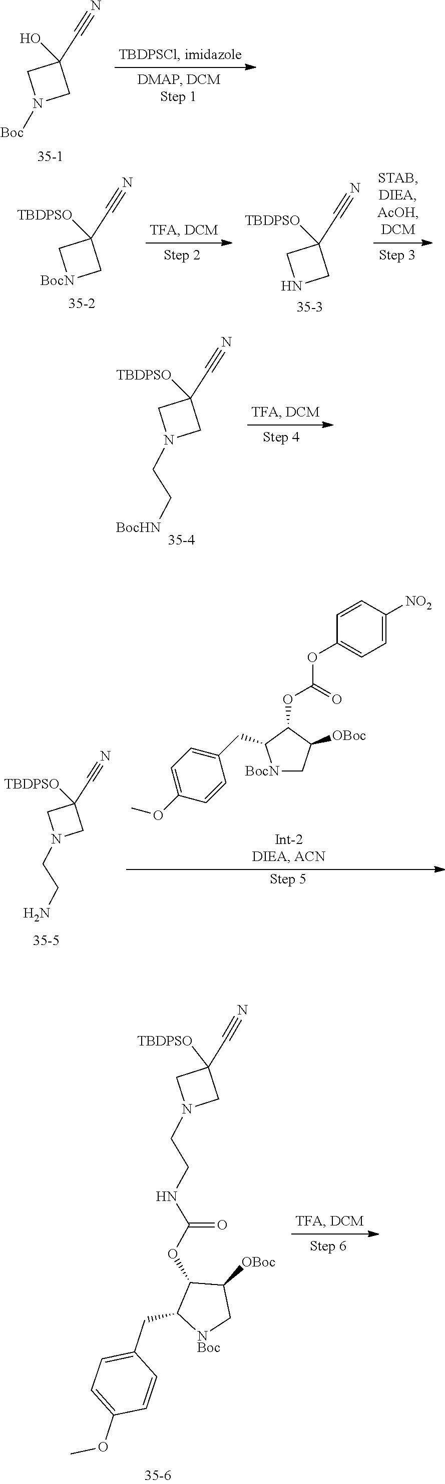

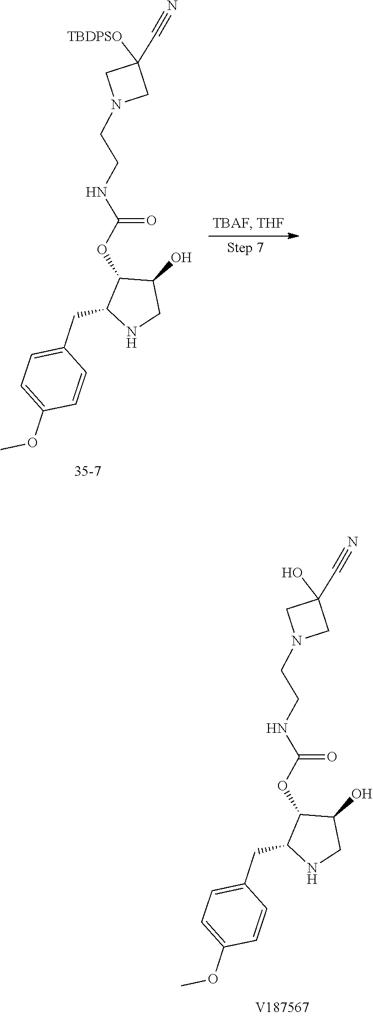

Step 1: To a stirred solution of tert-butyl 3-cyano-3-hydroxyazetidine-1-carboxylate (35-1, 300 mg, 1.51 mmol, 1.0 eq.) in DCM (10 mL) was added 1H-imidazole (309.1 mg, 4.54 mmol, 3.0q.) and DMAP (184. mg, 1.51 mmol, 1.0 eq.) and TBDPSCl (832 mg, 3.03 mmol, 2.0 eq.) in portions at 0° C. The resulting mixture was stirred over night at 25° C. under a nitrogen atmosphere. The residue was purified by silica gel column chromatography, eluted with PE/EA (1:1) to afford tert-butyl 3-[(tert-butyldiphenylsilyl)oxy]-3-cyanoazetidine-1-carboxylate (500 mg, 75.7%) as a light yellow oil. MS: m/z: Calc'd for C₂₅H₃₂N₂O₃Si [M+H−56+41]⁺422. Found, 422.

Step 2: The compound was prepared in 93.2% yield as a light yellow oil according to Boc Deprotection; General Procedure II in STEP 2. MS: m/z: Calc'd for C₂₀H₂₄N₂O_{Si} [M+H]⁺337. Found, 337.

Step 3: To a stirred solution of 3-[(tert-butyldiphenylsilyl)oxy]azetidine-3-carbonitrile (35-3, 200 mg, 0.59 mmol, 1.0 eq.) in DCM (4 mL) was added DIEA (230.5 mg, 1.78 mmol, 3.0 eq.) in portions at room temperature stirred 5 min. Then add tert-butyl N-(2-oxoethyl)carbamate (189.2 mg, 1.19 mmol, 2.0 eq.) and AcOH (178.5 mg, 2.97 mmol, 5.0 eq.) stir 0.5 h at 25° C. Then add NaBH(AcO)₃ (377.9 mg, 1.78 mmol, 3.0 eq.) at 0° C. The resulting mixture was stirred over night at 25° C. Desired product could be detected by LCMS. The resulting mixture was concentrated under reduced pressure. The residue was purified by reversed-phase flash chromatography to afford tert-butyl N-(2-{3-[(tert-butyldiphenylsilyl)oxy]-3-cyanoazetidine-1-yl}ethyl)carbamate (150 mg, 52.6%) as a light yellow oil. MS: m/z: Calc'd for C₂₇H₃₇N₃O₃Si [M+H]⁺480. Found, 480.

Step 4: The compound was prepared in 89.3% yield as a light yellow oil according to Boc Deprotection; General Procedure II in STEP 2. MS: m/z: Calc'd for C₂₂H₂₉N₃O_{Si} [M+H]⁺380. Found, 380.

### Step 5 and 6

The compound 70-7 was prepared in 76.2% overall yield as a light yellow solid according to Substitution reaction for synthesis of Carbamate; General Procedure I using 1-(2-aminoethyl)-3-[(tert-butyldiphenylsilyl)oxy]azetidine-3-carbonitrile (70-6) in STEP 5; Boc Deprotection; General Procedure II in STEP 6. MS: m/z Calc'd for C₃₅H₄₄N₄O₅Si [M+H]⁺629. Found, 629.

Step 7: To a stirred solution of (2R,3S,4S)-4-hydroxy-2-[(4-methoxyphenyl)methyl]pyrrolidin-3-yl N-(2-{3-[(tert-butyldiphenylsilyl)oxy]-3-cyanoazetidine-1-yl}ethyl)carbamate (35-7, 60 mg, 0.10 mmol, 1.0 eq.) in THF (1 mL) was added TBAF (49.9 mg, 0.19 mmol, 2.0 eq.) in portions at room temperature. The resulting mixture was stirred 1 h at 25° C. The crude product (60 mg) was purified by Prep-HPLC to afford (2R,3S,4S)-4-hydroxy-2-[(4-methoxyphenyl)methyl]pyrrolidin-3-yl N-[2-(3-cyano-3-hydroxyazetidin-1-yl)ethyl]carbamate (1.4 mg, 2.8%) as a light yellow oil. MS: m/z: Calc'd for C₁₉H₂₆N₄O₅ [M+H]⁺391. Found, 391. ¹H NMR (400 MHz, Methanol-d₄) δ 7.28-7.20 (m, 2H), 6.98-6.90 (m, 2H), 5.00 (d, J=3.5 Hz, 1H), 4.64-4.60 (m, 2H), 4.44 (d, J=4.6 Hz, 1H), 4.23-4.08 (m, 3H), 3.80 (s, 3H), 3.57 (dd, J=12.6, 4.3 Hz, 1H), 3.49-3.38 (m, 2H), 3.37 (s, 2H), 3.22 (d, J=12.6 Hz, 1H), 3.12 (dd, J=14.2, 6.6 Hz, 1H), 2.98 (dd, J=14.3, 8.9 Hz, 1H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.67.

### Synthetic Procedure of (2R,3S,4S)-4-hydroxy-2-[(4-methoxyphenyl)methyl]pyrrolidin-3-yl N-[(4-carbamoylphenyl)methyl]carbamate (V188296)

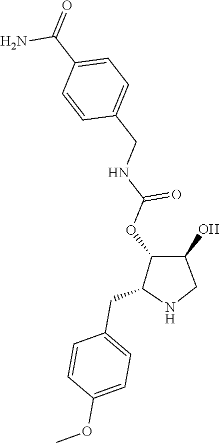 [SMILES: COc1ccc(C[C@H]2NC[C@H](O)[C@H]2OC(=O)NCc2ccc(C(N)=O)cc2)cc1]

The title compound was prepared in 42.7% overall yield as a white solid according to Substitution reaction for synthesis of Carbamate; General Procedure I using 4-(aminomethyl)benzamide in STEP 1; Boc Deprotection; General Procedure II in STEP 2. MS: m/z Calc'd for C₂₁H₂₅N₃O₅ [M+H]⁺400. Found, 400. ¹H NMR (400 MHz, Methanol-d₄) δ 7.90 (d, J=8.3 Hz, 2H), 7.45 (d, J=8.0 Hz, 2H), 7.20 (d, J=8.5 Hz, 2H), 6.87 (dd, J=8.8, 2.2 Hz, 2H), 4.94 (d, J=3.4 Hz, 1H), 4.49-4.32 (m, 3H), 4.20-4.16 (m, 1H), 3.79 (d, J=0.9 Hz, 3H), 3.60 (dd, J=12.5, 4.5 Hz, 1H), 3.24 (d, J=12.6 Hz, 1H), 3.11 (dd, J=14.0, 7.7 Hz, 1H), 2.99 (dd, J=13.9, 8.1 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 29% B in 10 min; Wave Length: 254 nm/220 nm; RT1(min): 9.8.

### Synthetic Procedure of (2R,3S,4S)-4-hydroxy-2-[(4-methoxyphenyl)methyl]pyrrolidin-3-yl N-[(3-cyano-4-fluorophenyl)methyl]carbamate (V188310)

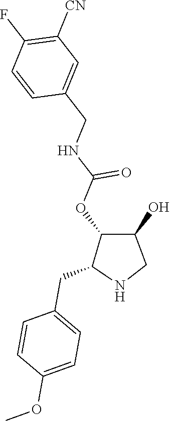 [SMILES: COc1ccc(C[C@H]2NC[C@H](O)[C@H]2OC(=O)NCc2ccc(F)c(C#N)c2)cc1]

The title compound was prepared in 34.9% overall yield as a white solid according to Substitution reaction for synthesis of Carbamate; General Procedure I using 5-(aminomethyl)-2-fluorobenzonitrile in STEP 1; Boc Deprotection; General Procedure II in STEP 2. MS: m/z Calc'd for C₂₁H₂₂FN₃O₄[M+H]⁺400. Found, 400. ¹H NMR (400 MHz, Methanol-d₄) δ 7.77-7.66 (m, 2H), 7.40-7.36 (m, 1H), 7.24-7.17 (m, 2H), 6.93-6.85 (m, 2H), 4.94 (d, J=3.6 Hz, 1H), 4.49-4.28 (m, 3H), 4.18-4.16 (m, 1H), 3.80 (s, 3H), 3.59 (dd, J=12.6, 4.3 Hz, 1H), 3.23 (d, J=12.6 Hz, 1H), 3.11 (dd, J=14.1, 7.6 Hz, 1H), 2.98 (dd, J=14.0, 8.2 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 7% B to 37% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-(3-phenylprop-2-yn-1-yl)carbamate (V188315)

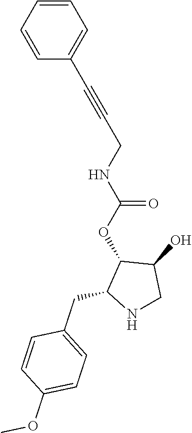 [SMILES: COc1ccc(C[C@H]2NC[C@H](O)[C@H]2OC(=O)NCC#Cc2ccccc2)cc1]

The title compound was prepared in 45.1% overall yield as a white solid according to Substitution reaction for synthesis of Carbamate; General Procedure I using 3-phenylprop-2-yn-1-amine in STEP 1; Boc Deprotection; General Procedure II in STEP 2. MS: m/z Calc'd for C₂₂H₂₄N₂O₄ [M+H]⁺381. Found, 381. ¹H NMR (400 MHz, Methanol-d₄) δ 7.47-7.30 (m, 5H), 7.28-7.20 (m, 2H), 6.95-6.84 (m, 2H), 4.97 (d, J=3.5 Hz, 1H), 4.44-4.39 (m, 1H), 4.23-4.10 (m, 3H), 3.73 (s, 3H), 3.59 (dd, J=12.5, 4.3 Hz, 1H), 3.23 (d, J=12.5 Hz, 1H), 3.14 (dd, J=14.0, 7.4 Hz, 1H), 2.99 (dd, J=14.1, 8.3 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 11% B to 41% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-[(4-cyclopropylphenyl)methyl]carbamate (V188321)

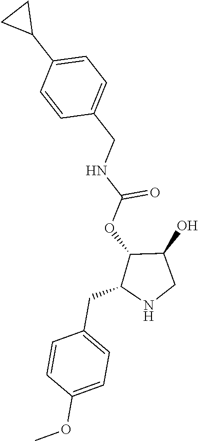 [SMILES: COc1ccc(C[C@H]2NC[C@H](O)[C@H]2OC(=O)NCc2ccc(C3CC3)cc2)cc1]

The title compound was prepared in 45.7% overall yield as a white solid according to Substitution reaction for synthesis of Carbamate; General Procedure I using 1-(4-cyclopropylphenyl)methanamine in STEP 1; Boc Deprotection; General Procedure II in STEP 2. MS: m/z Calc'd for C₂₃H₂₈N₂O₄ [M+H]⁺397. Found, 397. ¹H NMR (400 MHz, Methanol-d₄) δ 7.26-6.99 (m, 6H), 6.86 (d, J=8.2 Hz, 2H), 4.92 (s, 1H), 4.41-4.21 (m, 3H), 4.17-4.13 (m, 1H), 3.79 (s, 3H), 3.06-3.56 (m, 1H), 3.22 (d, J=12.6 Hz, 1H), 3.09-3.07 (m, 1H), 3.02-2.91 (m, 1H), 1.96-1.88 (m, 1H), 1.01-0.92 (m, 2H), 0.71-0.60 (m, 2H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-{2-[(2S)-pyrrolidin-2-yl]ethyl}carbamate (V188341)

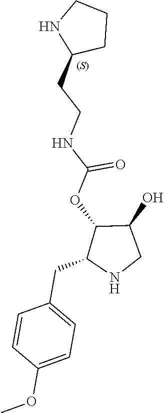

The title compound was prepared in 45.7% overall yield as a white solid according to Substitution reaction for synthesis of Carbamate; General Procedure I using tert-butyl (2S)-2-(2-aminoethyl)pyrrolidine-1-carboxylate in STEP 1; Boc Deprotection; General Procedure II in STEP 2. MS: m/z Calc'd for C₁₉H₂₉N₃O₄ [M+H]⁺364. Found, 364. ¹H NMR (400 MHz, Methanol-d₄) δ 7.26-7.18 (m, 2H), 6.95-6.87 (m, 2H), 4.94 (d, J=4.0 Hz, 1H), 4.36 (d, J=4.2 Hz, 1H), 4.18-4.14 (m, 1H), 3.78 (s, 3H), 3.61-3.47 (m, 2H), 3.34 (d, J=7.7 Hz, 1H), 3.29-3.25 (m, J=6.9, 3.0 Hz, 3H), 3.20-3.18 (m, 1H), 3.09 (dd, J=14.2, 7.1 Hz, 1H), 2.97 (dd, J=14.1, 8.5 Hz, 1H), 2.32-2.24 (m, 1H), 2.17-1.84 (m, 4H), 1.74-1.64 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 19% B in 10 min; Wave Length: 254 nm/220 nm; RT1(min): 9.8.

### Synthetic Procedure of (2R,3S,4S)-4-hydroxy-2-[(4-methoxyphenyl)methyl]pyrrolidin-3-yl N-(1,2-oxazol-4-ylmethyl)carbamate (V188383)

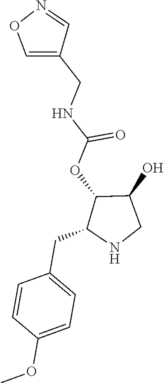 [SMILES: COc1ccc(C[C@H]2NC[C@H](O)[C@H]2OC(=O)NCc2cnoc2)cc1]

The title compound was prepared in 45.7% overall yield as an off-white solid according to Substitution reaction for synthesis of Carbamate; General Procedure I using 1-(1,2-oxazol-4-yl)methanamine in STEP 1; Boc Deprotection; General Procedure II in STEP 2. MS: m/z Calc'd for C₁₇H₂₁N₃O₅ [M+H]⁺348. Found, 348. ¹H NMR (400 MHz, Methanol-d₄) δ 8.66 (s, 1H), 8.44 (s, 1H), 7.22-7.14 (m, 2H), 6.91-6.84 (m, 2H), 4.39 (d, J=4.2 Hz, 1H), 4.32-4.21 (m, 2H), 4.20-4.14 (m, 1H), 3.80 (s, 3H), 3.61-3.52 (m, 1H), 3.23 (d, J=12.6 Hz, 1H), 3.09 (dd, J=14.1, 7.6 Hz, 1H), 3.02-2.92 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 32% B in 10 min; Wave Length: 254 nm/220 nm; RT1(min): 9.5.

### Synthetic Procedure of (2R,3S,4S)-4-hydroxy-2-[(4-methoxyphenyl)methyl]pyrrolidin-3-yl N-{[2-(pyrrolidin-1-yl)pyridin-4-yl]methyl}carbamate (V188226)

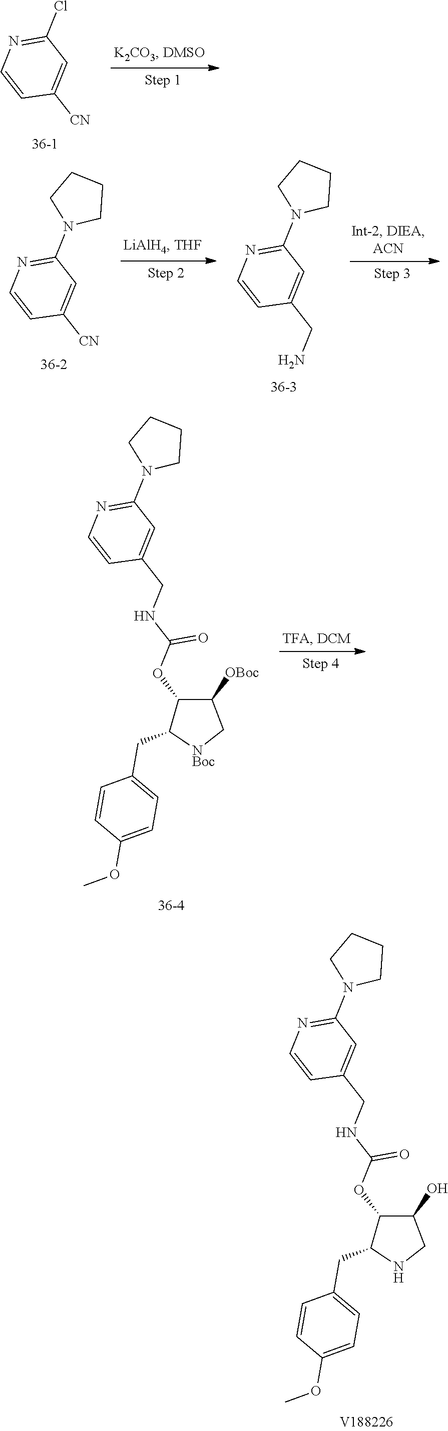

Step 1: To a stirred mixture of 2-chloropyridine-4-carbonitrile (36-1, 500 mg, 3.60 mmol, 1 equiv) and pyrrolidine (513.3 mg, 7.21 mmol, 2 equiv) in DMSO (10 mL) was added K₂CO₃ (1496.2 mg, 10.82 mmol, 3 equiv) at room temperature. The resulting mixture was stirred at 100° C. for overnight. The resulting mixture was filtered. the filter cake was washed with DMSO (1×2 mL). The residue was purified by reversed-phase flash chromatography to afford 2-(pyrrolidin-1-yl)pyridine-4-carbonitrile (600 mg, 95.9%) as a yellow solid. MS: m/z: Calc'd for C₁₀H₁₁N₃ [M+H]⁺174. found 174.

Step 2: To a stirred mixture of 2-(pyrrolidin-1-yl)pyridine-4-carbonitrile (36-2, 870 mg, 5.02 mmol, 1 equiv) in THF (6 mL) was added LiAlH₄ (4 mL, 10.04 mmol, 2 equiv) at 0° C. under nitrogen atmosphere. The resulting mixture was stirred for overnight at 80° C. under nitrogen atmosphere. The mixture was allowed to cool down to 0° C. The reaction was quenched with CAS:7727-73-3 and EA (30 ml) at 0° C. The resulting mixture was filtered. the filter cake was washed with EtOAc (2×20 mL). The filtrate was concentrated under reduced pressure. to afford 1-[2-(pyrrolidin-1-yl)pyridin-4-yl]methanamine (700 mg, 78.6%) as a yellow oil. MS: m/z: Calc'd for C₁₀H₁₅N₃[M+H]⁺178. found 178.

### Step 3 and 4

The title compound was prepared in 26.1% overall yield as an off-white solid according to Substitution reaction for synthesis of Carbamate; General Procedure I using 1-[2-(pyrrolidin-1-yl)pyridin-4-yl]methanamine in STEP 3; Boc Deprotection; General Procedure II in STEP 4. MS: m/z Calc'd for C₂₃H₃₀N₄O₄[M+H]⁺427. found 427. ¹H NMR (400 MHz, Methanol-d₄) δ 7.85 (d, J=6.7 Hz, 1H), 7.27-7.20 (m, 2H), 7.03-6.79 (m, 4H), 5.01 (d, J=3.6 Hz, 1H), 4.55-4.31 (m, 3H), 4.18-4.02 (m, 1H), 3.79 (s, 3H), 3.66-3.54 (m, 5H), 3.24 (d, J=12.6 Hz, 1H), 3.14 (dd, J=14.2, 7.0 Hz, 1H), 3.01 (dd, J=14.1, 8.6 Hz, 1H), 2.19-2.03 (m, 4H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 27% B in 10 min; Wave Length: 254 nm/220 nm; RT1(min): 9.88.

### Synthetic Procedure of (2R,3S,4S)-4-hydroxy-2-[(4-methoxyphenyl)methyl]pyrrolidin-3-yl N-{[2,6-bis(pyrrolidin-1-yl)pyridin-4-yl]methyl}carbamate (V188228)

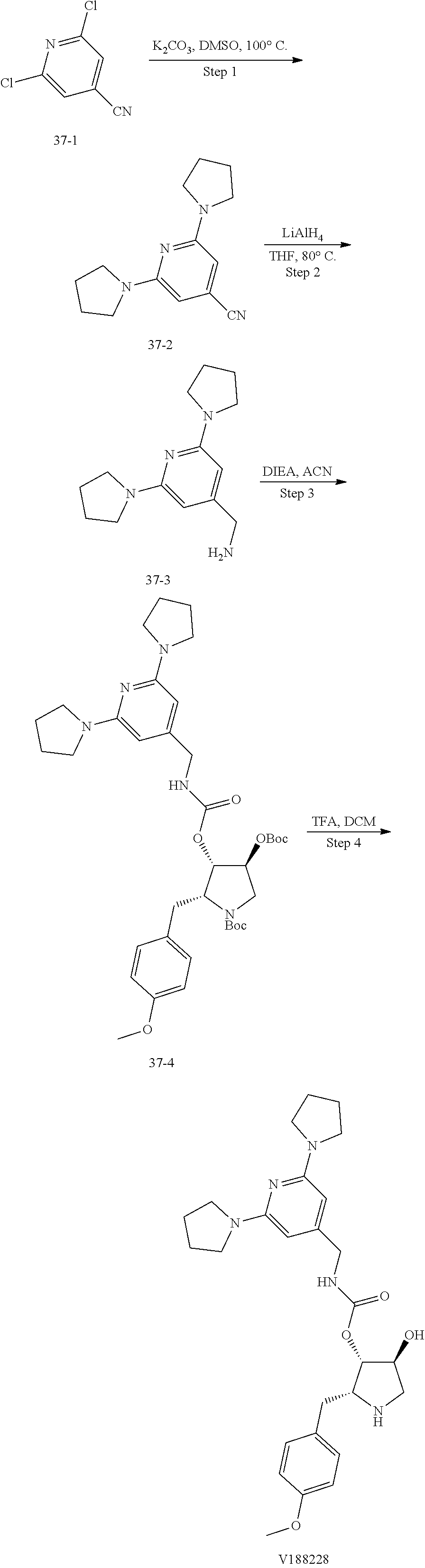

Step 1: To a stirred mixture of 2,6-dichloropyridine-4-carbonitrile (37-1, 300 mg, 1.73 mmol, 1 equiv) and pyrrolidine (246.6 mg, 3.46 mmol, 2 equiv) in DMSO (8 mL) was added K₂CO₃ (718.9 mg, 5.20 mmol, 3 equiv) at room temperature. The resulting mixture was stirred at 100° C. for overnight. The resulting mixture was filtered. the filter cake was washed with DMSO (2 mL). The residue was purified by reversed-phase flash chromatography to afford 2,6-bis(pyrrolidin-1-yl)pyridine-4-carbonitrile (400 mg, 95.1%) as a green oil. MS: m/z: Calc'd for C₁₄H₁₈N₄ [M+H]⁺243, found 243.

Step 2: To a stirred mixture of 2,6-bis(pyrrolidin-1-yl)pyridine-4-carbonitrile (37-2, 400 mg, 1.651 mmol, 1 equiv) in THF (6 mL) was added LiAlH₄ (1.3 mL, 10.046 mmol. 2 equiv) at 0° C. under nitrogen atmosphere. The resulting mixture was stirred for overnight at 80° C. under nitrogen atmosphere. The mixture was allowed to cool down to 0° C. The reaction was quenched with CAS:7727-73-3 and EA (30 ml) at 0° C. The resulting mixture was filtered. the filter cake was washed with EtOAc (2×20 mL). The filtrate was concentrated under reduced pressure. to afford 1-[2,6-bis(pyrrolidin-1-yl)pyridin-4-yl]methanamine (300 mg, 73.7%) as a yellow oil. MS: m/z: Calc'd for C₁₄H₂₂N₄[M+H]⁺247. found 247.

Step 3 and 4: The title compound was prepared in 35.5% overall yield as a yellow solid according to Substitution reaction for synthesis of Carbamate; General Procedure I using 1-[2,6-bis(pyrrolidin-1-yl)pyridin-4-yl]methanamine (37-3) in STEP 3; Boc Deprotection; General Procedure II in STEP 4. MS: m/z Calc'd for C₂₇H₃₇N₅O₄ [M+H]⁺496. found 496. ¹H NMR (400 MHz, DMSO-d₆) δ 9.44-9.42 (m, 2H), 7.81-7.69 (m, 1H), 7.31-6.78 (m, 4H), 5.71-5.69 (m, 2H), 4.76-4.74 (m, 1H), 4.25 (d, J=3.9 Hz, 1H), 4.17 (d, J=5.8 Hz, 1H), 4.12-4.01 (m, 1H), 4.05 (d, J=6.0 Hz, 1H), 4.01 (s, 1H), 3.97-3.93 (m, 1H), 3.91 (s, 1H), 3.73 (s, 3H), 3.39 (d, J=6.1 Hz, 9H), 3.14-2.79 (m, 3H), 1.89-1.72 (m, 8H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 32% B in 10 min; Wave Length: 254 nm/220 nm; RT1(min): 10.17.

### Synthetic Procedure of (2R,3S,4S)-4-hydroxy-2-[(4-methoxyphenyl)methyl]pyrrolidin-3-yl N-[(4-sulfamoylphenyl)methyl]carbamate (V188304)

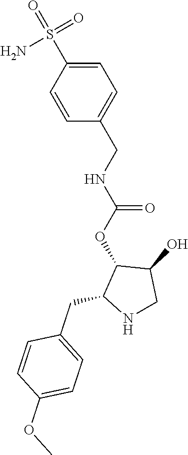 [SMILES: COc1ccc(C[C@H]2NC[C@H](O)[C@H]2OC(=O)NCc2ccc(S(N)(=O)=O)cc2)cc1]

The title compound was prepared in 46.4% overall yield as an off-white solid according to Substitution reaction for synthesis of Carbamate; General Procedure I using emilene in STEP 1; Boc Deprotection; General Procedure II in STEP 2. MS: m/z Calc'd for C₂₀H₂₅N₃O₆S [M+H]⁺436, found 436. ¹H NMR (400 MHz, Methanol-d₄) δ 7.95-7.87 (m, 2H), 7.51 (d, J=8.3 Hz, 2H), 7.23-7.09 (m, 2H), 6.92-6.84 (m, 2H), 4.93 (d, J=3.6 Hz, 1H), 4.52-4.34 (m, 3H), 4.17-4.02 (m, 1H), 3.79 (s, 3H), 3.68-3.53 (m, 1H), 3.23 (d, J=12.6 Hz, 1H), 3.11 (dd, J=14.1, 7.7 Hz, 1H), 2.99 (dd, J=13.9, 7.6 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 3% B to 33% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-[2-(2-aminopyridin-4-yl)ethyl]carbamate (V188307)

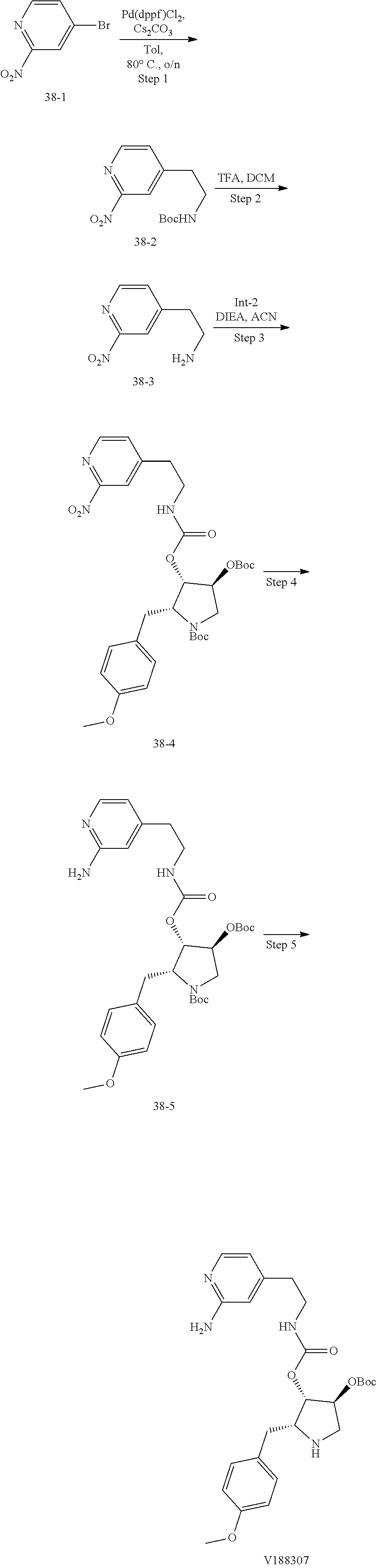

Step 1: To a stirred mixture of 4-bromo-2-nitropyridine (38-1, 300 mg, 1.47 mmol, 1 equiv) and tert-butyl N-[2-(trifluoroboranuidyl)ethyl]carbamate (375.9 mg, 1.77 mmol, 1.2 equiv) in Toluene (8 mL) and H₂O (2 mL) was added 1,1′-bis(diphenylphosphino)ferrocene-palladium(II (120.3 mg, 0.14 mmol, 0.1 equiv) and Cs₂CO₃ (1444.55 mg, 4.434 mmol, 3 equiv) at room temperature. The reaction was placed under vacuum, sonicated and backfilled with nitrogen. The resulting mixture was stirred at 80° C. for overnight. The resulting mixture was filtered, the filter cake was washed with MeCN (2×2 mL). The filtrate was concentrated under reduced pressure. The residue was dissolved in MeCN (5 mL). The residue was purified by reversed-phase flash chromatography to afford tert-butyl N-[2-(2-nitropyridin-4-yl)ethyl]carbamate (300 mg, 75.9%) as a yellow oil. MS: m/z: Calc'd for C₁₂H₁₇N₃O₄ [M+H]⁺268. found 268.

Step 2: To a stirred mixture of tert-butyl N-[2-(2-nitropyridin-4-yl)ethyl]carbamate (200 mg, 0.74 mmol, 1 equiv) in DCM (5 mL) was added TFA (1 mL) at room temperature. The resulting mixture was stirred at room temperature for 2 h. The resulting mixture was concentrated under reduced pressure. to afford 2-(2-nitropyridin-4-yl)ethanamine (100 mg, 79.9%) as a yellow oil. MS: m/z: Calc'd for C₇H₉N₃O₂[M+H]⁺168. found 168.

Step 3: The compound was prepared in 76.3% yield as a yellow solid according to Substitution reaction for synthesis of Carbamate; General Procedure I using 2-(2-nitropyridin-4-yl)ethanamine (38-3). MS: m/z Calc'd for C₃₀H₄₀N₄O₁₀ [M+22]⁺639. found 639.

Step 4: To a stirred mixture of tert-butyl (2R,3S,4S)-4-[(tert-butoxycarbonyl)oxy]-2-[(4-methoxyphenyl)methyl]-3-({[2-(2-nitropyridin-4-yl)ethyl]carbamoyl}oxy)pyrrolidine-1-carboxylate (38-4, 100 mg, 0.16 mmol, 1 equiv) in AcOH (10 mL) was added Fe (54.3 mg, 0.97 mmol, 6 equiv) at room temperature. The resulting mixture was stirred for overnight at 80° C. under nitrogen atmosphere. The resulting mixture was concentrated under reduced pressure. The residue was dissolved in MeCN (5 mL). The residue was purified by reversed-phase flash chromatography to afford tert-butyl (2R,3S,4S)-3-({[2-(2-aminopyridin-4-yl)ethyl]carbamoyl}oxy)-4-[(tert-butoxycarbonyl)oxy]-2-[(4-methoxyphenyl)methyl]pyrrolidine-1-carboxylate (80 mg, 84.0%) as a yellow oil. MS: m/z: Calc'd for C₃₀H₄₂N₄O₈ [M+H]⁺587. found 587.

Step 5: The title compound was prepared in 45.8% yield as a yellow solid according to Boc Deprotection; General Procedure II. MS: m/z Calc'd for C₂₀H₂₆N₄O₄ [M+H]⁺387. found 387. ¹H NMR (400 MHz, Methanol-d₄) δ 7.74 (d, J=6.6 Hz, 1H), 7.23-7.13 (m, 2H), 6.94-6.80 (m, 4H), 4.92 (d, J=3.4 Hz, 1H), 4.36-4.30 (m, 1H), 4.12-4.01 (m, 1H), 3.77 (s, 3H), 3.50-3.38 (m, 3H), 3.18 (d, J=12.6 Hz, 1H), 3.05 (dd, J=14.1, 7.0 Hz, 1H), 2.96-2.83 (m, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 23% B in 10 min; Wave Length: 254 nm/220 nm; RT1(min): 9.1.

### Synthetic Procedure of (2R,3S,4S)-4-hydroxy-2-[(4-methoxyphenyl)methyl]pyrrolidin-3-yl N-{2-[(2R)-pyrrolidin-2-yl]ethyl}carbamate (V188342)

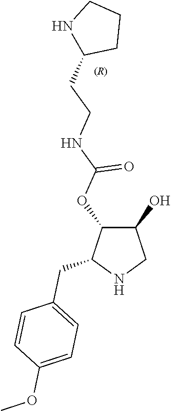

The title compound was prepared in 63.7% overall yield as a white solid according to Substitution reaction for synthesis of Carbamate; General Procedure I using tert-butyl (2R)-2-(2-aminoethyl)pyrrolidine-1-carboxylate in STEP 1; Boc Deprotection; General Procedure II in STEP 2. MS: m/z Calc'd for C₁₉H₂₉N₃O₄ [M+H]⁺364. found 364. ¹H NMR (400 MHz, Methanol-d₄) δ 7.26-7.17 (m, 2H), 6.92-6.86 (m, 2H), 4.97-4.92 (m, 1H), 4.40-4.34 (m, 1H), 4.15-4.02 (m, 1H), 3.78 (s, 3H), 3.62-3.50 (m, 2H), 3.36-3.31 (m, 1H), 3.32-3.24 (m, 3H), 3.20 (d, J=12.7 Hz, 1H), 3.09 (dd, J=14.2, 7.1 Hz, 1H), 2.96 (dd, J=14.1, 8.5 Hz, 1H), 2.34-2.22 (m, 1H), 2.17-1.84 (m, 4H), 1.69-1.52 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 9.3.

### Synthetic Procedure of (2R,3S,4S)-4-hydroxy-2-[(4-methoxyphenyl)methyl]pyrrolidin-3-yl N-{2-[(3R)-pyrrolidin-3-yloxy]ethyl}carbamate (V188071)

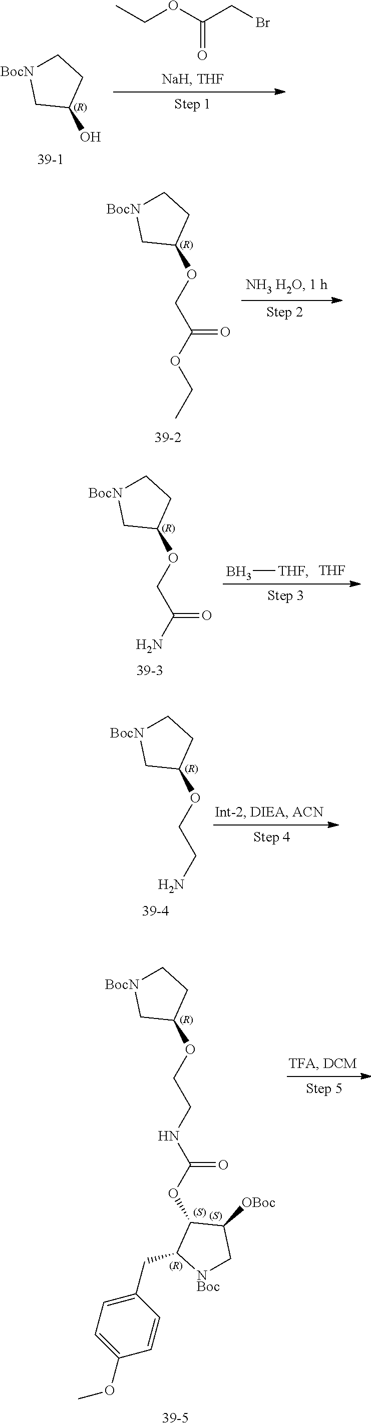

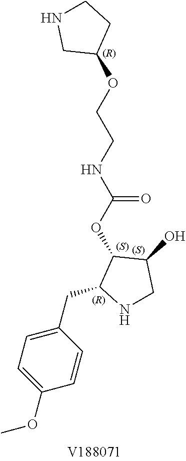

Step 1: To a stirred solution of NaH (0.3 g, 16.02 mmol, 3 eq.) in THF (10 mL) were added tert-butyl (3R)-3-hydroxypyrrolidine-1-carboxylate (1 g, 5.34 mmol, 1 eq.) and ethyl bromoacetate (1.7 g, 10.68 mmol, 2 eq.) at 0° C. The resulting mixture was stirred at room temperature for overnight. The crude product was purified by reversed phase flash to afford tert-butyl (3R)-3-(2-ethoxy-2-oxoethoxy)pyrrolidine-1-carboxylate (760 mg, 52.0%) as a light yellow oil. MS: m/z: Calc'd for C₁₃H₂₃NO₅ [M+H−56]⁺218. Found, 218.

Step 2: To a stirred solution of tert-butyl (3R)-3-(2-ethoxy-2-oxoethoxy)pyrrolidine-1-carboxylate (750 mg, 2.74 mmol, 1 eq.) in NH₃·H₂O (8 mL) at room temperature. The resulting mixture was stirred at room temperature for 1 h. The resulting mixture was concentrated under reduced pressure to afford crude product tert-butyl (3R)-3-(carbamoylmethoxy)pyrrolidine-1-carboxylate (700 mg) as a light yellow oil. MS: m/z: Calc'd for C₁₁H₂₀N₂O₄[M+H]⁺245. Found, 245.

Step 3: To a stirred solution of tert-butyl (3R)-3-(carbamoylmethoxy)pyrrolidine-1-carboxylate (250 mg, 1.02 mmol, 1 eq.) in THF (2 mL) was added BH₃-THF (4 mL) at 0° C. The resulting mixture was stirred at room temperature for overnight. The resulting mixture was concentrated under reduced pressure to afford tert-butyl (3R)-3-(2-aminoethoxy)pyrrolidine-1-carboxylate (50 mg, 21.2%) as a light yellow oil. MS: m/z: Calc'd for C₁₁H₂₂N₂O₃ [M+H]⁺231. Found, 231.

### Step 4 and 5

The title compound was prepared in 61.1% overall yield as a white solid according to Substitution reaction for synthesis of Carbamate; General Procedure I using tert-butyl (3R)-3-(2-aminoethoxy)pyrrolidine-1-carboxylate (39-4) in STEP 4; Boc Deprotection; General Procedure II in STEP 5. MS: m/z Calc'd for C₂₂H₂₈N₄O₄ [M+H]⁺413. Found, 413. ¹H NMR (400 MHz, Methanol-d₄) δ 7.22-7.11 (m, 2H), 6.89-6.77 (m, 2H), 4.69-4.64 (m, 1H), 4.22-4.12 (m, 2H), 3.78 (s, 3H), 3.55 (s, 3H), 3.60-3.53 (m, 1H), 3.51-3.43 (m, 1H), 3.31 (d, J=3.5 Hz, 1H), 3.20-3.08 (m, 2H), 3.01 (dd, J=12.4, 4.6 Hz, 2H), 2.89-2.70 (m, 3H), 1.97 (dd, J=13.6, 10.3 Hz, 2H).

Prep-HPLC purification conditions: Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 5% B to 25% B in 10 min; Wave Length: 254 nm/220 nm; RT1(min): 6.7.

### Synthetic Procedure of (2R,3S,4S)-4-hydroxy-2-[(4-methoxyphenyl)methyl]pyrrolidin-3-yl N-{[2,6-bis(azetidin-1-yl)pyridin-4-yl]methyl}carbamate (V188227)

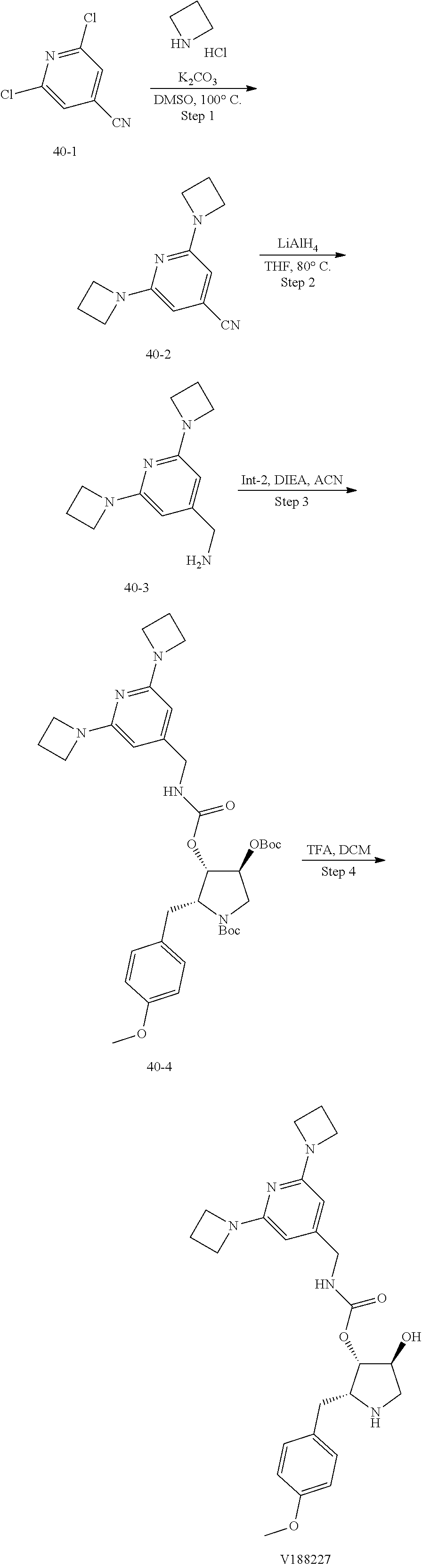

Step 1: To a stirred solution of 2,6-dichloropyridine-4-carbonitrile (300 mg, 1.73 mmol, 1 eq.) and azetidine hydrochloride (324.4 mg, 3.46 mmol, 2 eq.) in DMSO (5 mL) were added K₂CO₃ (718.9 mg, 5.20 mmol, 3 equiv) at 0° C. The resulting mixture was stirred at 100° C. for overnight. The crude product was purified by reversed phase flash to afford 2,6-bis(azetidin-1-yl)pyridine-4-carbonitrile (100 mg, 26.9%) as a light yellow solid. MS: m/z: Calc'd for C₁₂H₁₄N₄[M+H]⁺215. Found, 215.

Step 2: To a stirred solution of 2,6-bis(azetidin-1-yl)pyridine-4-carbonitrile (95 mg, 0.44 mmol, 1 eq.) in THF (3 mL) was added LiAlH₄ (33.6 mg, 0.88 mmol, 2 eq.) at 0° C. The resulting mixture was stirred at 80° C. for 2 h. The crude product was purified by Prep-HPLC to afford 1-[2,6-bis(azetidin-1-yl)pyridin-4-yl]methanamine (58 mg, 59.9%) as a light yellow solid. MS: m/z: Calc'd for C₁₂H₁₈N₄[M+H]⁺219. Found, 219.

Step 3 and 4: The title compound was prepared in 38.4% overall yield as a white solid according to Substitution reaction for synthesis of Carbamate; General Procedure I using 1-[2,6-bis(azetidin-1-yl)pyridin-4-yl]methanamine (40-3) in STEP 3; Boc Deprotection; General Procedure II in STEP 4. MS: m/z Calc'd for C₂₅H₃₃N₅O₄ [M+H]⁺468. Found, 468. ¹H NMR (400 MHz, Methanol-d₄) δ 9.58 (s, 1H), 9.19 (s, 1H), 7.90-7.71 (t, J=6.2 Hz, 1H), 7.26-7.18 (m, 2H), 6.91-6.84 (m, 2H), 5.64-5.59 (m, 2H), 4.78-4.70 (m, 1H), 4.24-4.22 (m, 1H), 4.18-4.07 (m, 3H), 4.07-3.99 (m, 8H), 3.74 (s, 3H), 3.39 (s, 2H), 3.09 (s, 2H), 3.04-2.81 (m, 4H), 2.28 (d, J=7.4 Hz, 4H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 23% B to 53% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-(oxetan-3-ylmethyl)carbamate (V188299)

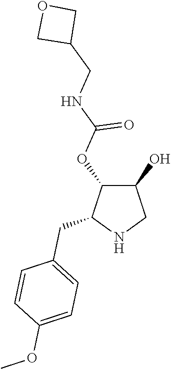 [SMILES: COc1ccc(C[C@H]2NC[C@H](O)[C@H]2OC(=O)NCC2COC2)cc1]

The title compound was prepared in 6.2% overall yield as a white solid according to Substitution reaction for synthesis of Carbamate; General Procedure I using 1-(oxetan-3-yl)methanamine in STEP 1; Boc Deprotection; General Procedure II in STEP 2. MS: m/z Calc'd for C₁₇H₂₄N₂O₅[M+H]⁺337. Found, 337. ¹H NMR (400 MHz, Methanol-d₄) δ 7.17-7.10 (m, 2H), 6.89-6.81 (m, 2H), 4.81-4.79 (m, 2H), 4.62-4.60 (m, 1H), 4.48-4.46 (m, 2H), 4.18-4.11 (m, 1H), 3.78 (s, 3H), 3.68 (s, 1H), 3.60-3.48 (m, 2H), 2.70 (m, 1H), 3.26-3.14 (m, 1H), 2.88-2.70 (m, 3H).

Prep-HPLC purification conditions: Column: Torus Diol OBD 3*25 cm, 5 um; Mobile Phase A: CO2, Mobile Phase B: IPA (1%-2M-NH₃-MeOH); Flow rate: 65 mL/min; Gradient: isocratic 34% B; Column Temperature (° C.): 35; Back Pressure (bar): 100; Wave Length: 220 nm; RT1(min): 6.6; Sample Solvent: IPA+DCM; Injection Volume: 1.5 mL.

### Synthetic Procedure of (2R,3S,4S)-4-hydroxy-2-[(4-methoxyphenyl)methyl]pyrrolidin-3-yl N-[(5-methyl-1,3,4-oxadiazol-2-yl)methyl]carbamate (V188303)

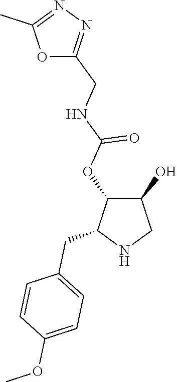 [SMILES: COc1ccc(C[C@H]2NC[C@H](O)[C@H]2OC(=O)NCc2nnc(C)o2)cc1]

The title compound was prepared in 24.1% overall yield as a white solid according to Substitution reaction for synthesis of Carbamate; General Procedure I using 1-(5-methyl-1,3,4-oxadiazol-2-yl)methanamine in STEP 1; Boc Deprotection; General Procedure II in STEP 2. MS: m/z Calc'd for C₁₇H₂₂N₄O₅[M+H]⁺363. Found, 363. ¹H NMR (400 MHz, Methanol-d₄) δ 7.25 (s, 2H), 6.97-6.89 (m, 2H), 5.01-4.95 (m, 1H), 4.65-4.51 (m, 2H), 4.43 (d, J=4.5 Hz, 1H), 4.22-4.13 (m, 1H), 3.80 (s, 3H), 3.61 (dd, J=12.6, 4.3 Hz, 1H), 3.23 (d, J=12.7 Hz, 1H), 3.14 (dd, J=14.1, 7.2 Hz, 1H), 2.99 (dd, J=14.2, 8.5 Hz, 1H), 2.55 (s, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 27% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-{[4-(methylcarbamoyl)phenyl]methyl}carbamate (V188305)

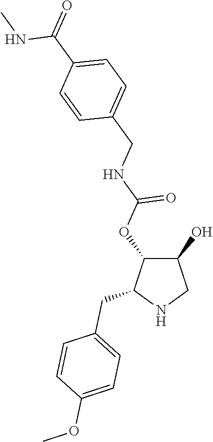 [SMILES: CNC(=O)c1ccc(CNC(=O)O[C@@H]2[C@@H](O)CN[C@@H]2Cc2ccc(OC)cc2)cc1]

The title compound was prepared in 25.5% overall yield as a white solid according to Substitution reaction for synthesis of Carbamate; General Procedure I using 4-(aminomethyl)-N-methylbenzamide in STEP 1; Boc Deprotection; General Procedure II in STEP 2. MS: m/z Calc'd for C₂₂H₂₇N₃O₅[M+H]⁺414. Found, 414. ¹H NMR (400 MHz, Methanol-d₄) δ 7.87-7.80 (m, 2H), 7.44 (d, J=8.1 Hz, 2H), 7.24-7.15 (m, 2H), 6.90-6.83 (m, 2H), 4.93 (d, J=4.2 Hz, 1H), 4.51-4.32 (m, 3H), 4.17-4.15 (m, 1H), 3.78 (s, 3H), 3.60 (dd, J=12.5, 4.2 Hz, 1H), 3.23 (d, J=12.6 Hz, 1H), 3.11 (dd, J=14.0, 7.7 Hz, 1H), 2.99 (dd, J=14.0, 8.2 Hz, 1H), 2.93 (s, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 3% B to 33% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-[(6-aminopyridazine-3-yl)methyl]carbamate (V188311)

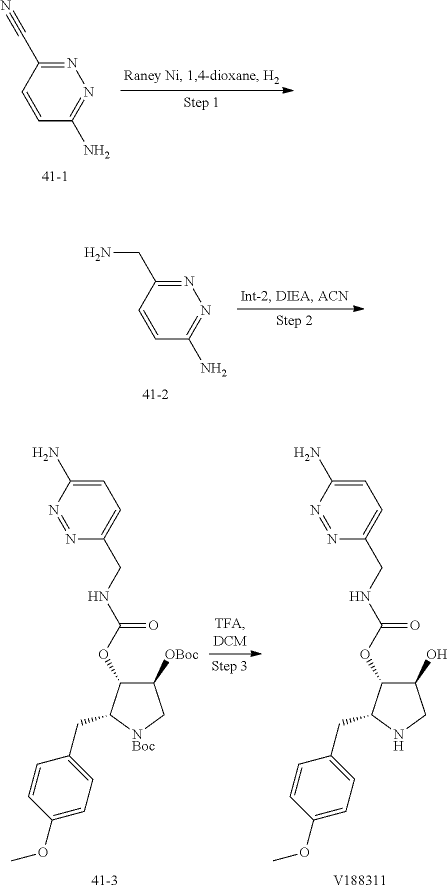

Step 1: To a stirred solution of 6-aminopyridazine-3-carbonitrile (150 mg, 1.24 mmol, 1 eq.) in 1,4-dioxane (4 mL) was added Raney Nickel (50 mg, 0.58 mmol) at 0° C. The resulting mixture was stirred at 60° C. under hydrogen atmosphere for overnight. The resulting mixture was filtered, the filter cake was washed with 1,4-dioxane (1×30 mL). The filtrate was concentrated under reduced pressure to afford 6-(aminomethyl)pyridazin-3-amine (150 mg, 96.7%) as a yellow oil. MS: m/z: Calc'd for C₅H₈N₄ [M+H]⁺125. Found, 125.

Step 2 and 3: The title compound was prepared in 15.8% overall yield as a white solid according to Substitution reaction for synthesis of Carbamate; General Procedure I using 6-(aminomethyl)pyridazin-3-amine (41-2) in STEP 2; Boc Deprotection; General Procedure II in STEP 3. MS: m/z Calc'd for C₁₈H₂₃N₅O₄ [M+H]⁺374. Found, 374. ¹H NMR (400 MHz, Methanol-d₄) δ 7.37 (d, J=9.2 Hz, 1H), 7.12 (d, J=8.3 Hz, 2H), 6.98 (t, J=9.5 Hz, 1H), 6.89-6.78 (m, 2H), 4.62 (d, J=3.8 Hz, 1H), 4.48-4.33 (m, 2H), 4.16 (d, J=5.3 Hz, 1H), 3.77 (s, 3H), 3.69-3.58 (m, 1H), 3.56-3.49 (m, 1H), 2.80-2.73 (dd, J=7.6, 3.4 Hz, 3H).

Prep-HPLC purification conditions: Column: XBridge Prep OBD C18 Column, 30*150 mm, 5 μm; Mobile Phase A: Water (10 nmol/LNH₄HCO₃), Mobile Phase B: ACN; Flow rate: 60 mL/min; Gradient: 2% B to 24% B in 10 min; Wave Length: 254 nm/220 nm; RT1(min): 8.72.

### Synthetic Procedure of (2R,3S,4S)-4-hydroxy-2-[(4-methoxyphenyl)methyl]pyrrolidin-3-yl N-{[4-(pyrazol-1-ylmethyl)phenyl]methyl}carbamate (V188314)

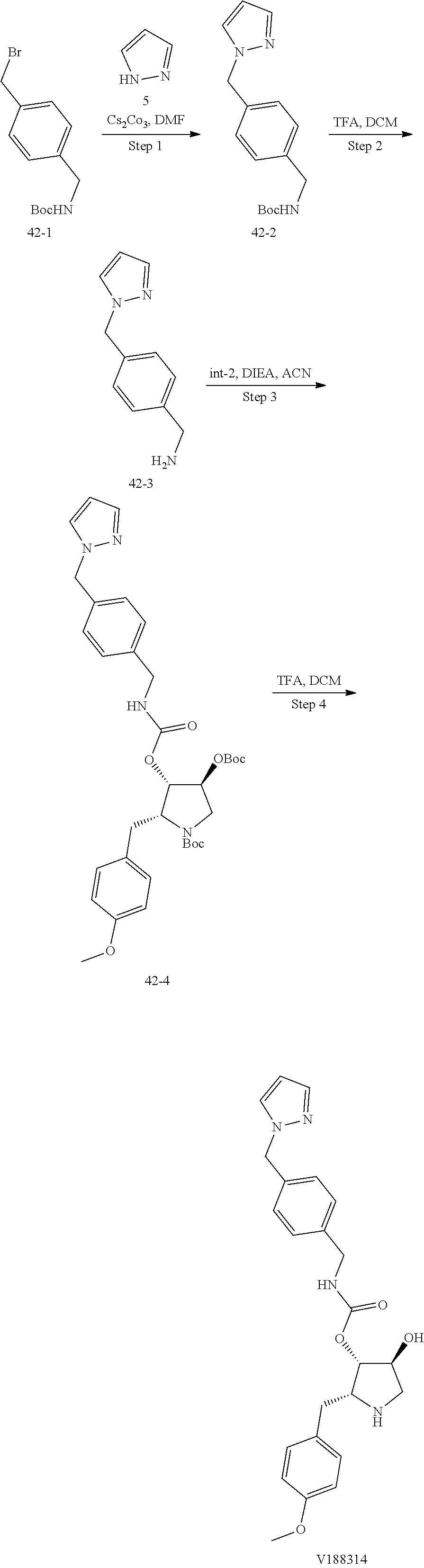

Step 1: To a stirred solution of tert-butyl N-{[4-(bromomethyl)phenyl]methyl}carbamate (300 mg, 0.99 mmol, 1 eq.) and Cs₂CO₃ (976.8 mg, 2.99 mmol, 3 eq.) in DMF (4 mL) was added pyrazole (136.0 mg, 1.99 mmol, 2 eq.) at 0° C. After completion, the resulting mixture was stirred at room temperature for 2 h. The crude product was purified by reversed phase flash to afford tert-butyl N-{[4-(pyrazol-1-ylmethyl)phenyl]methyl}carbamate (250 mg, 87.0%) as a light yellow oil. MS: m/z: Calc'd for C₁₆H₂₁N₃O₂[M+H]⁺288. Found, 288.

Step 2: To a stirred solution of tert-butyl N-{[4-(pyrazol-1-ylmethyl)phenyl]methyl}carbamate (250 mg, 0.87 mmol, 1 eq.) in DCM (3 mL) was added TFA (0.6 mL) at 0° C. The resulting mixture was stirred at room temperature for 2 h. The resulting mixture was concentrated under reduced pressure to afford 1-[4-(pyrazol-1-ylmethyl)phenyl]methanamine as a light yellow solid which was used directly in the next step without further purification. MS: m/z: Calc'd for C₁₁H₁₃N₃[M+H]⁺ 188. Found, 188.

Step 3 and 4: The title compound was prepared in 45.8% overall yield as a white solid according to Substitution reaction for synthesis of Carbamate; General Procedure I using 1-[4-(pyrazol-1-ylmethyl)phenyl]methanamine (42-3) in STEP 3; Boc Deprotection; General Procedure II in STEP 4. MS: m/z Calc'd for C₂₄H₂₈N₄O₄ [M+H]⁺437. Found, 437. ¹H NMR (400 MHz, Methanol-d₄) δ 7.70 (d, J=2.3 Hz, 1H), 7.53 (d, J=1.9 Hz, 1H), 7.32 (d, J=8.0 Hz, 2H), 7.21 (dd, J=14.1, 8.3 Hz, 4H), 6.93-6.84 (m, 2H), 6.34 (t, J=2.2 Hz, 1H), 5.36 (s, 2H), 4.94 (d, J=3.3 Hz, 1H), 4.37-4.29 (m, 3H), 4.16-4.14 (m, 1H), 3.79 (s, 3H), 3.56 (dd, J=12.6, 4.3 Hz, 1H), 3.22 (d, J=12.6 Hz, 1H), 3.09 (dd, J=14.1, 7.6 Hz, 1H), 2.95 (dd, J=14.0, 8.3 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 11% B to 41% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-[(6-aminopyridin-3-yl)methyl]carbamate (V188316)

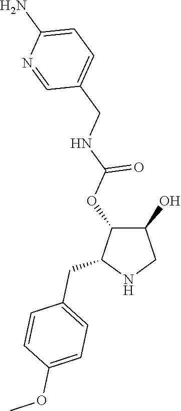 [SMILES: COc1ccc(C[C@H]2NC[C@H](O)[C@H]2OC(=O)NCc2ccc(N)nc2)cc1]

The title compound was prepared in 40.3% overall yield as a white solid according to Substitution reaction for synthesis of Carbamate; General Procedure I using 5-(aminomethyl)pyridin-2-amine in STEP 1; Boc Deprotection; General Procedure II in STEP 2. MS: m/z Calc'd for C₁₉H₂₄N₄O₄[M+H]⁺373. Found, 373. ¹H NMR (400 MHz, Methanol-d₄) δ 7.93 (dd, J=9.2, 2.1 Hz, 1H), 7.81 (d, J=2.1 Hz, 1H), 7.23-7.16 (m, 2H), 7.03 (d, J=9.2 Hz, 1H), 6.93-6.85 (m, 2H), 4.95 (d, J=3.6 Hz, 1H), 4.39 (d, J=4.2 Hz, 1H), 4.27-4.13 (m, 3H), 3.79 (s, 3H), 3.57 (dd, J=12.6, 4.3 Hz, 1H), 3.23 (d, J=12.6 Hz, 1H), 3.09 (dd, J=14.1, 7.3 Hz, 1H), 2.97 (dd, J=14.1, 8.4 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 23% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-(cyclobutylmethyl)carbamate (V188318)

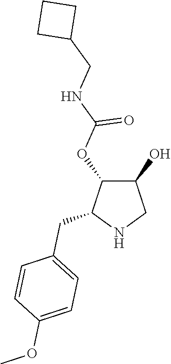 [SMILES: COc1ccc(C[C@H]2NC[C@H](O)[C@H]2OC(=O)NCC2CCC2)cc1]

The title compound was prepared in 60.2% overall yield as a white solid according to Substitution reaction for synthesis of Carbamate; General Procedure I using 1-cyclobutylmethanamine in STEP 1; Boc Deprotection; General Procedure II in STEP 2. MS: m/z Calc'd for C₁₈H₂₆N₂O₄[M+H]⁺335. Found, 335. ¹H NMR (400 MHz, Methanol-d₄) δ 7.27-7.18 (m, 2H), 6.97-6.88 (m, 2H), 4.92-4.90 (m, 1H), 4.37-4.35 (m, 1H), 4.13-4.11 (m, 1H), 3.80 (s, 3H), 3.55-3.53 (m, 1H), 3.27-3.06 (m, 4H), 2.95 (dd, J=14.0, 8.4 Hz, 1H), 2.54 (d, J=7.5 Hz, 1H), 2.10 (s, 2H), 2.02-1.84 (m, 2H), 1.83-1.70 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 12% B to 42% B in 10 min; Wave Length: 254 nm/220 nm; RT1(min): 9.5.

### Synthetic Procedure of (2R,3S,4S)-4-hydroxy-2-[(4-methoxyphenyl)methyl]pyrrolidin-3-yl N-{[4-(aminomethyl)phenyl]methyl}carbamate (V188322)

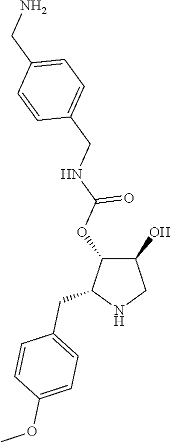 [SMILES: COc1ccc(C[C@H]2NC[C@H](O)[C@H]2OC(=O)NCc2ccc(CN)cc2)cc1]

The title compound was prepared in 57.0% overall yield as a white solid according to Substitution reaction for synthesis of Carbamate; General Procedure I using tert-butyl N-{[4-(aminomethyl)phenyl]methyl}carbamate in STEP 1; Boc Deprotection; General Procedure II in STEP 2. MS: m/z Calc'd for C₂₁H₂₇N₃O₄[M+H]⁺386. Found, 386. ¹H NMR (400 MHz, Methanol-d₄) δ 7.50-7.40 (m, 4H), 7.25-7.19 (m, 2H), 6.94-6.86 (m, 2H), 4.95 (d, J=3.6 Hz, 1H), 4.47-4.34 (m, 3H), 4.20-4.13 (m, 3H), 3.80 (s, 3H), 3.58 (dd, J=12.6, 4.3 Hz, 1H), 3.22 (d, J=12.6 Hz, 1H), 3.10 (dd, J=14.1, 7.4 Hz, 1H), 2.97 (dd, J=14.0, 8.3 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 23% B in 10 min; Wave Length: 254 nm/220 nm; RT1(min): 9.5.

### Synthetic Procedure of (2R,3S,4S)-4-hydroxy-2-[(4-methoxyphenyl)methyl]pyrrolidin-3-yl N-(1,2,3,4-tetrahydroisoquinolin-6-ylmethyl)carbamate (V188345)

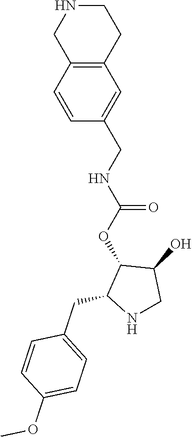 [SMILES: COc1ccc(C[C@H]2NC[C@H](O)[C@H]2OC(=O)NCc2ccc3c(c2)CCNC3)cc1]

The title compound was prepared in 21.7% overall yield as a white solid according to Substitution reaction for synthesis of Carbamate; General Procedure I using tert-butyl 6-(aminomethyl)-3,4-dihydro-1H-isoquinoline-2-carboxylate in STEP 1; Boc Deprotection; General Procedure II in STEP 2. MS: m/z Calc'd for C₂₃H₂₉N₃O₄[M+H]⁺412. Found, 412. ¹H NMR (400 MHz, Methanol-d₄) δ 7.31-7.19 (m, 5H), 6.94-6.85 (m, 2H), 4.98-4.93 (m, 1H), 4.42-4.29 (m, 5H), 4.17-4.15 (m, 1H), 3.80 (s, 3H), 3.59 (dd, J=12.6, 4.3 Hz, 1H), 3.51 (t, J=6.4 Hz, 2H), 3.23 (d, J=12.6 Hz, 1H), 3.11 (dd, J=14.4, 6.7 Hz, 3H), 2.97 (dd, J=14.0, 8.4 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 26% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-(isoquinolin-6-ylmethyl)carbamate (V188348)

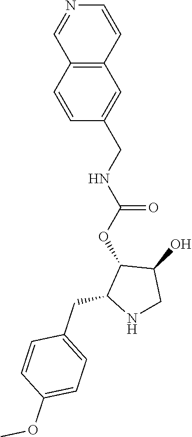 [SMILES: COc1ccc(C[C@H]2NC[C@H](O)[C@H]2OC(=O)NCc2ccc3cnccc3c2)cc1]

The title compound was prepared in 43.3% overall yield as a white solid according to Substitution reaction for synthesis of Carbamate; General Procedure I using 1-(isoquinolin-6-yl)methanamine in STEP 1; Boc Deprotection; General Procedure II in STEP 2. MS: m/z Calc'd for C₂₃H₂₅N₃O₄[M+H]⁺408. Found, 408. ¹H NMR (400 MHz, Methanol-d₄) δ 9.65 (s, 1H), 8.57 (d, J=6.5 Hz, 1H), 8.45 (d, J=8.6 Hz, 1H), 8.32 (d, J=6.4 Hz, 1H), 8.15 (s, 1H), 7.97 (dd, J=8.6, 1.6 Hz, 1H), 7.26-7.19 (m, 2H), 6.96-6.79 (m, 2H), 5.02-4.96 (m, 1H), 4.75-4.60 (m, 2H), 4.44 (d, J=4.1 Hz, 1H), 4.20-4.18 (m, 1H), 3.77 (s, 3H), 3.68-3.58 (m, 1H), 3.25 (d, J=12.7 Hz, 1H), 3.14 (dd, J=13.8, 7.6 Hz, 1H), 3.01 (dd, J=14.1, 8.4 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 23% B in 10 min; Wave Length: 254 nm/220 nm; RT1(min): 9.3.

### Synthetic Procedure of (2R,3S,4S)-4-hydroxy-2-[(4-methoxyphenyl)methyl]pyrrolidin-3-yl N-[(4-cyanophenyl)methyl]carbamate (V188377)

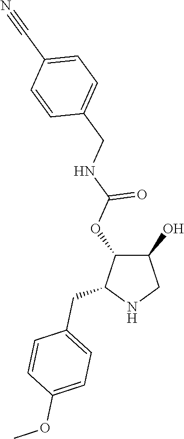 [SMILES: COc1ccc(C[C@H]2NC[C@H](O)[C@H]2OC(=O)NCc2ccc(C#N)cc2)cc1]

The title compound was prepared in 38.1% overall yield as a white solid according to Substitution reaction for synthesis of Carbamate; General Procedure I using 4-(aminomethyl)benzonitrile in STEP 1; Boc Deprotection; General Procedure II in STEP 2. MS: m/z Calc'd for C₂₁H₂₃N₃O₄[M+H]⁺382. Found, 382. ¹H NMR (400 MHz, Methanol-d₄) δ 7.77-7.71 (m, 2H), 7.52 (d, J=8.2 Hz, 2H), 7.23-7.15 (m, 2H), 6.92-6.84 (m, 2H), 4.93 (s, 1H), 4.52-4.34 (m, 3H), 4.12-4.10 (m, 1H), 3.79 (s, 3H), 3.55 (dd, J=12.6, 4.4 Hz, 1H), 3.19 (d, J=12.6 Hz, 1H), 3.09 (dd, J=14.0, 7.5 Hz, 1H), 2.95 (dd, J=14.0, 8.2 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 10% B to 40% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-[(3-fluorophenyl)methyl]carbamate (V188379)

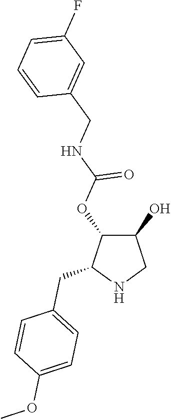 [SMILES: COc1ccc(C[C@H]2NC[C@H](O)[C@H]2OC(=O)NCc2cccc(F)c2)cc1]

The title compound was prepared in 38.8% overall yield as a white solid according to Substitution reaction for synthesis of Carbamate; General Procedure I using 1-(3-fluorophenyl)methanamine in STEP 1; Boc Deprotection; General Procedure II in STEP 2. MS: m/z Calc'd for C₂₀H₂₃FN₂O₄[M+H]⁺375. Found, 375. ¹H NMR (400 MHz, Methanol-d₄) δ 7.38-7.36 (m, 1H), 7.24-6.99 (m, 5H), 6.92-6.84 (m, 2H), 4.9 (s, 1H), 4.44-4.35 (m, 3H), 4.13-4.11 (m, 1H), 3.79 (s, 3H), 3.56 (dd, J=12.6, 4.4 Hz, 1H), 3.20 (d, J=12.6 Hz, 1H), 3.09 (dd, J=14.0, 7.7 Hz, 1H), 2.96 (dd, J=14.0, 8.1 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 13% B to 43% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-[(3,4-difluorophenyl)methyl]carbamate (V190408)

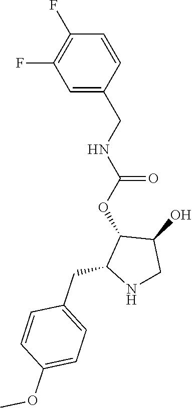 [SMILES: COc1ccc(C[C@H]2NC[C@H](O)[C@H]2OC(=O)NCc2ccc(F)c(F)c2)cc1]

The title compound was prepared in 57.8% overall yield as a white solid according to Substitution reaction for synthesis of Carbamate; General Procedure I using 1-(3,4-difluorophenyl)methanamine in STEP 1; Boc Deprotection; General Procedure II in STEP 2. MS: m/z Calc'd for C₂₀H₂₂F₂N₂O₄[M+H]⁺393. Found, 393. ¹H NMR (400 MHz, Methanol-d₄) δ 7.32-7.11 (m, 5H), 6.93-6.85 (m, 2H), 4.94 (d, J=3.5 Hz, 1H), 4.40-4.28 (d, J=4.2 Hz, 3H), 4.17-4.15 (m, 1H), 3.80 (s, 3H), 3.58 (dd, J=12.6, 4.3 Hz, 1H), 3.23 (d, J=12.6 Hz, 1H), 3.11 (dd, J=14.0, 7.6 Hz, 1H), 2.97 (dd, J=14.0, 8.2 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 15% B to 45% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-{[4-(pyrrolidin-2-yl)phenyl]methyl}carbamate (V188355)

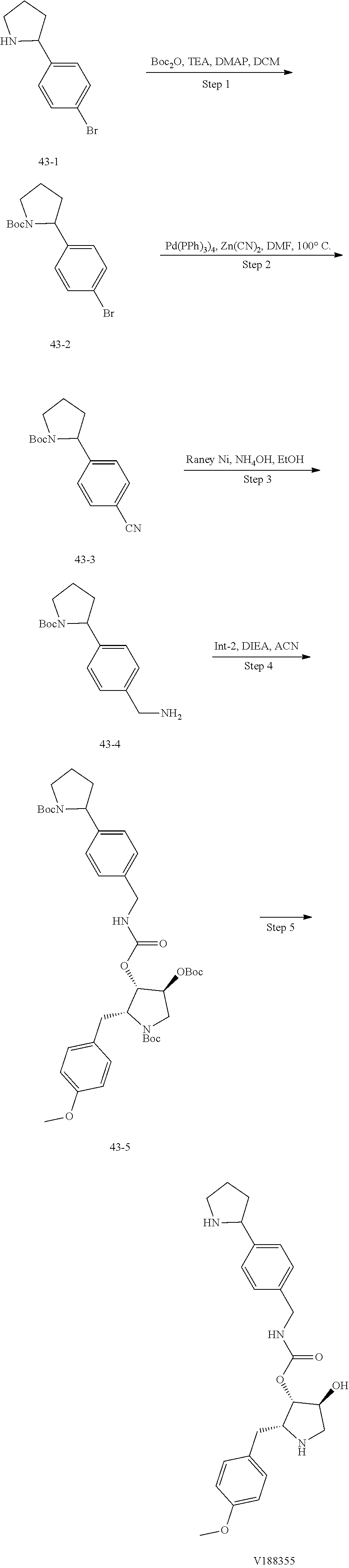

Step 1: To the solution of 2-(4-bromophenyl)pyrrolidine (300 mg, 1.33 mmol, 1 eq.), DMAP (16 mg, 0.13 mmol, 0.1 eq.) and TEA (269 mg, 2.65 mmol, 2 eq.) in DCM (5 mL) was added Boc₂O (619 mg, 2.65 mmol, 2 eq.) slowly. The result mixture was stirred at rt for overnight. The mixture was concentrated to give the crude product which was purified by Prep TLC (PE:EA=5:1 as eluent). The tert-butyl 2-(4-bromophenyl)pyrrolidine-1-carboxylate (240 mg, 55.5%) was obtained as a white solid. MS: m/z: Calc'd for C₁₅H₂₀BrNO₂ [M+H]⁺326. found 326.

Step 2: To the solution of tert-butyl 2-(4-bromophenyl)pyrrolidine-1-carboxylate (110 mg, 0.34 mmol, 1 eq.) and Zn(CN)₂ (63 mg, 0.54 mmol, 1.6 eq.) in DMF (3 mL) was added Pd(PPh₃)₄ (39 mg, 0.03 mmol, 0.1 eq.). The mixture was stirred at 100° C. for overnight under N₂. The mixture was filtered and the filtrate was purified by reversed phase flash column to afford tert-butyl 2-(4-cyanophenyl)pyrrolidine-1-carboxylate (90 mg, 98.0%) was obtained as a white solid. MS: m/z: Calc'd for C₁₆H₂₀N₂O₂ [M+H−56]⁺217. found 217.

Step 3: To the solution of tert-butyl 2-(4-cyanophenyl)pyrrolidine-1-carboxylate (90 mg, 0.33 mmol, 1 eq.) in MeOH (3 mL) was added Raney Ni (20 mg, 0.23 mmol, 0.71 eq.) and 2 drops NH₄OH. The reaction was placed under vacuum, sonicated and backfilled with hydrogen. The mixture was stirred at rt overnight. The mixture was filtered and the filtrate was concentrated to give the crude product which was used in the next step without further purification. MS: m/z: Calc'd for C₁₆H₂₄N₂O₂ [2M+H]⁺553. found 553.

Step 4 and 5: The title compound was prepared in 60.2% overall yield as a white solid according to Substitution reaction for synthesis of Carbamate; General Procedure I using tert-butyl 2-[4-(aminomethyl)phenyl]pyrrolidine-1-carboxylate (43-4) in STEP 4; Boc Deprotection; General Procedure II in STEP 5. MS: m/z Calc'd for C₂₄H₃₁N₃O₄[M+H]⁺426. Found, 426. ¹H NMR (400 MHz, Methanol-d₄) δ 7.48 (d, J=8.3 Hz, 4H), 7.25-7.18 (m, 2H), 6.93-6.85 (m, 2H), 4.95 (d, J=3.5 Hz, 1H), 4.64 (t, J=8.2 Hz, 1H), 4.46-4.37 (m, 3H), 4.16-4.13 (m, 1H), 3.79 (s, 3H), 3.67-3.54 (m, 1H), 3.45 (t, J=6.7 Hz, 2H), 3.23 (d, J=12.6 Hz, 1H), 3.10 (dd, J=14.0, 7.4 Hz, 1H), 2.97 (dd, J=14.0, 8.4 Hz, 1H), 2.55-2.44 (m, 1H), 2.34-2.15 (m, 3H).

Prep-HPLC purification conditions: Column: Xselect CSH Prep C18 OBD Column, 30*150 mm, 5 μm; Mobile Phase A: Water (0.05% TFA), Mobile Phase B: ACN; Flow rate: 60 mL/min; Gradient: 3% B to 23% B in 7 min; Wave Length: 254 nm/220 nm; RT1(min): 5.82.

### Synthetic Procedure of (2R,3S,4S)-4-hydroxy-2-[(4-methoxyphenyl)methyl]pyrrolidin-3-yl N-[2-(3,5-dimethyl-1,2-oxazol-4-yl)ethyl]carbamate (V190414)

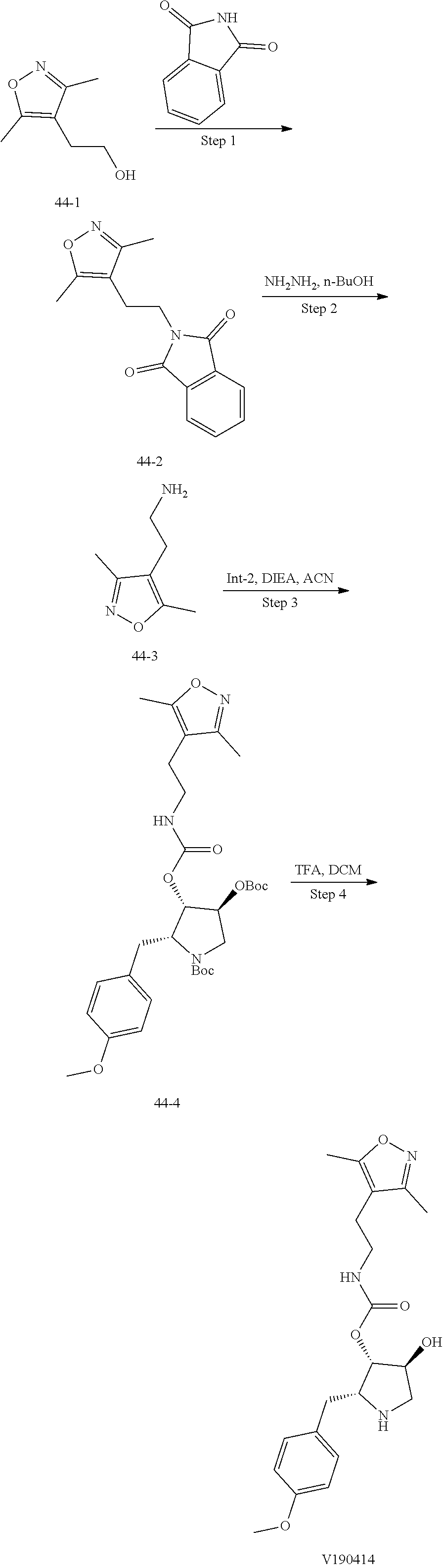

Step 1: To a stirred solution of 2-(3,5-dimethyl-1,2-oxazol-4-yl)ethanol (500 mg, 3.54 mmol, 1 eq.) and phthalimide (781.6 mg, 5.31 mmol, 1.5 eq.) in THF (10 mL) were added DTBAD (1631.1 mg, 7.08 mmol, 2 eq.) and PPH₃ (1857.9 mg, 7.08 mmol, 2 eq.) at 0° C. The resulting mixture was stirred at room temperature under nitrogen atmosphere for overnight. The crude product was purified by reversed phase flash to afford 2-[2-(3,5-dimethyl-1,2-oxazol-4-yl)ethyl]isoindole-1,3-dione (510 mg, 53.2%) as a yellow oil. MS: m/z: Calc'd for C₁₅H₁₄N₂O₃[M+H]⁺271. Found, 271.

Step 2: To a stirred solution of 2-[2-(3,5-dimethyl-1,2-oxazol-4-yl)ethyl]isoindole-1,3-dione (500 mg, 1.85 mmol, 1 eq.) in n-BuOH (10 mL) was added hydrazine hydrate (463.0 mg, 9.25 mmol, 5 eq.) at room temperature. The resulting mixture was stirred at 100° C. for overnight. The resulting mixture was concentrated under reduced pressure. The crude product (500 mg) was purified by Prep-HPLC to afford 2-(3,5-dimethyl-1,2-oxazol-4-yl)ethanamine (500 mg, 192.8%) as a white solid. MS: m/z: Calc'd for C₇H₁₂N₂O[M+H]⁺141. Found, 141.

Step 3 and 4: The title compound was prepared in 27.9% overall yield as a white solid according to Substitution reaction for synthesis of Carbamate; General Procedure I using 2-(3,5-dimethyl-1,2-oxazol-4-yl)ethanamine (44-3) in STEP 3; Boc Deprotection; General Procedure II in STEP 4. MS: m/z Calc'd for C₂₀H₂₇N₃O₉ [M+H]⁺390. Found, 390. ¹H NMR (400 MHz, Methanol-d₄) δ 7.20 (d, J=8.2 Hz, 2H), 6.93 (d, J=8.2 Hz, 2H), 4.92 (d, J=3.4 Hz, 1H), 4.31 (d, J=4.2 Hz, 1H), 4.13-4.11 (m, 1H), 3.80 (s, 3H), 3.51 (dd, J=12.6, 4.5 Hz, 1H), 3.30 (d, J=6.7 Hz, 2H), 3.20 (d, J=12.6 Hz, 1H), 3.07 (dd, J=14.2, 7.0 Hz, 1H), 2.90 (dd, J=14.2, 8.5 Hz, 1H), 2.61 (t, J=6.9 Hz, 2H), 2.36 (s, 3H), 2.26 (s, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 32% B in 10 min; Wave Length: 254 nm/220 nm; RT1(min): 10.75.

### Synthetic Procedure of (2R,3S,4S)-4-hydroxy-2-[(4-methoxyphenyl)methyl]pyrrolidin-3-yl N-[2-(3,5-dimethylpyrazol-1-yl)ethyl]carbamate (V190415)

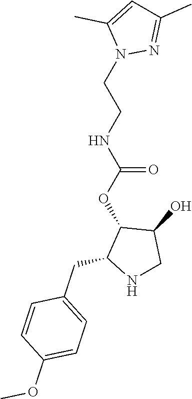

The title compound was prepared in 31.4% overall yield as a white solid according to Substitution reaction for synthesis of Carbamate; General Procedure I using 2-(3,5-dimethylpyrazol-1-yl)ethanamine in STEP 1; Boc Deprotection; General Procedure II in STEP 2. MS: m/z Calc'd for C₂₀H₂₈N₄O₄[M+H]⁺389. Found, 389. ¹H NMR (400 MHz, Methanol-d₄) δ 7.24-7.16 (m, 2H), 6.97-6.88 (m, 2H), 5.90 (s, 1H), 4.92 (d, J=3.2 Hz, 1H), 4.34 (d, J=4.3 Hz, 1H), 4.16 (t, J=6.0 Hz, 3H), 3.80 (s, 3H), 3.63-3.43 (m, 3H), 3.20 (d, J=12.6 Hz, 1H), 3.06 (dd, J=14.2, 6.8 Hz, 1H), 2.91 (dd, J=14.3, 8.7 Hz, 1H), 2.29 (s, 3H), 2.18 (s, 3H).

Prep-HPLC purification conditions: Column: Xselect CSH Prep C18 OBD Column, 30*150 mm, 5 μm; Mobile Phase A: Water (0.05% TFA), Mobile Phase B: ACN; Flow rate: 60 mL/min; Gradient: 3% B to 23% B in 7 min; Wave Length: 254 nm/220 nm; RT1(min): 5.82.

### Synthetic Procedure of (2R,3S,4S)-4-hydroxy-2-[(4-methoxyphenyl)methyl]pyrrolidin-3-yl N-[2-(1,2-oxazol-4-yl)ethyl]carbamate (V190449)

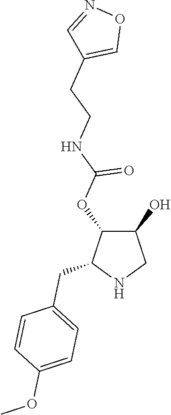 [SMILES: COc1ccc(C[C@H]2NC[C@H](O)[C@H]2OC(=O)NCCc2cnoc2)cc1]

The title compound was prepared in 42.3% overall yield as a white solid according to Substitution reaction for synthesis of Carbamate; General Procedure I using 2-(1,2-oxazol-4-yl)ethanamine in STEP 1; Boc Deprotection; General Procedure II in STEP 2. MS: m/z Calc'd for C₁₈H₂₃N₃O₅[M+H]⁺362. Found, 362. ¹H NMR (400 MHz, Methanol-d₄) δ 8.55 (s, 1H), 8.39 (s, 1H), 7.23-7.16 (m, 2H), 6.97-6.89 (m, 2H), 4.91 (d, J=3.5 Hz, 1H), 4.35 (d, J=4.1 Hz, 1H), 4.15-4.13 (m, 1H), 3.80 (s, 3H), 3.49-3.39 (m, 1H), 3.43-3.33 (m, 2H), 3.21 (d, J=12.6 Hz, 1H), 3.08 (dd, J=14.1, 7.2 Hz, 1H), 2.92 (dd, J=14.1, 8.4 Hz, 1H), 2.75 (t, J=6.8 Hz, 2H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 29% B in 10 min; Wave Length: 254 nm/220 nm; RT1(min): 10.38.

### Synthetic Procedure of (2R,3S,4S)-4-hydroxy-2-[(4-methoxyphenyl)methyl]pyrrolidin-3-yl N-[(2-fluoropyridin-4-yl)methyl]carbamate (V190522)

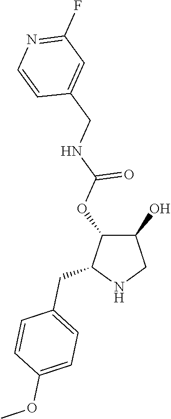 [SMILES: COc1ccc(C[C@H]2NC[C@H](O)[C@H]2OC(=O)NCc2ccnc(F)c2)cc1]

The title compound was prepared in 41.5% overall yield as a white solid according to Substitution reaction for synthesis of Carbamate; General Procedure I using 1-(2-fluoropyridin-4-yl)methanamine in STEP 1; Boc Deprotection; General Procedure II in STEP 2. MS: m/z Calc'd for C₁₉H₂₂FN₃O₄[M+H]⁺376. Found, 376. ¹H NMR (400 MHz, Methanol-d₄) δ 8.19 (d, J=5.2 Hz, 1H), 7.28-7.23 (d, J=8.2 Hz, 3H), 7.04 (s, 1H), 6.91 (d, J=8.2 Hz, 2H), 4.95 (d, J=3.5 Hz, 1H), 4.54-4.36 (m, 3H), 4.19-4.17 (m, 1H), 3.80 (s, 3H), 3.68-3.55 (m, 1H), 3.24 (d, J=12.6 Hz, 1H), 3.13 (dd, J=14.1, 7.5 Hz, 1H), 3.00 (dd, J=14.1, 8.2 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 32% B in 10 min; Wave Length: 254 nm/220 nm; RT1(min): 10.75.

### Synthetic Procedure of (2R,3S,4S)-4-hydroxy-2-[(4-methoxyphenyl)methyl]pyrrolidin-3-yl N-{6-azaspiro[3.4]octan-2-yl}carbamate (V190538)

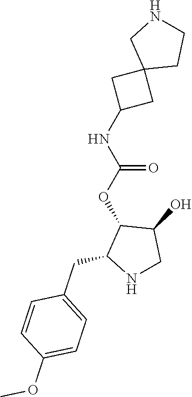 [SMILES: COc1ccc(C[C@H]2NC[C@H](O)[C@H]2OC(=O)NC2CC3(CCNC3)C2)cc1]

The title compound was prepared in 39.9% overall yield as a white solid according to Substitution reaction for synthesis of Carbamate; General Procedure I using tert-butyl 2-amino-6-azaspiro[3.4]octane-6-carboxylate in STEP 1; Boc Deprotection; General Procedure II in STEP 2. MS: m/z Calc'd for C₂₀H₂₉N₃O₄[M+H]⁺376. Found, 376. ¹H NMR (400 MHz, Methanol-d₄) δ 7.26-7.18 (m, 2H), 6.96-6.88 (m, 2H), 4.92 (d, J=3.6 Hz, 1H), 4.35 (d, J=4.2 Hz, 1H), 4.23-4.08 (m, 2H), 3.80 (s, 3H), 3.55 (dd, J=12.6, 4.3 Hz, 1H), 3.42 (s, 1H), 3.33-3.25 (m, 1H), 3.25-3.15 (m, 3H), 3.09 (dd, J=14.1, 7.2 Hz, 1H), 2.96 (dd, J=14.1, 8.5 Hz, 1H), 2.42-2.18 (m, 2H), 2.14 (m, 4H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 3% B to 33% B in 10 min; Wave Length: 254 nm/220 nm; RT1(min): 16.57.

### Synthetic Procedure of (2R,3S,4S)-4-hydroxy-2-[(4-methoxyphenyl)methyl]pyrrolidin-3-yl N-{2-[(2S)-piperidin-2-yl]ethyl}carbamate (V191902)

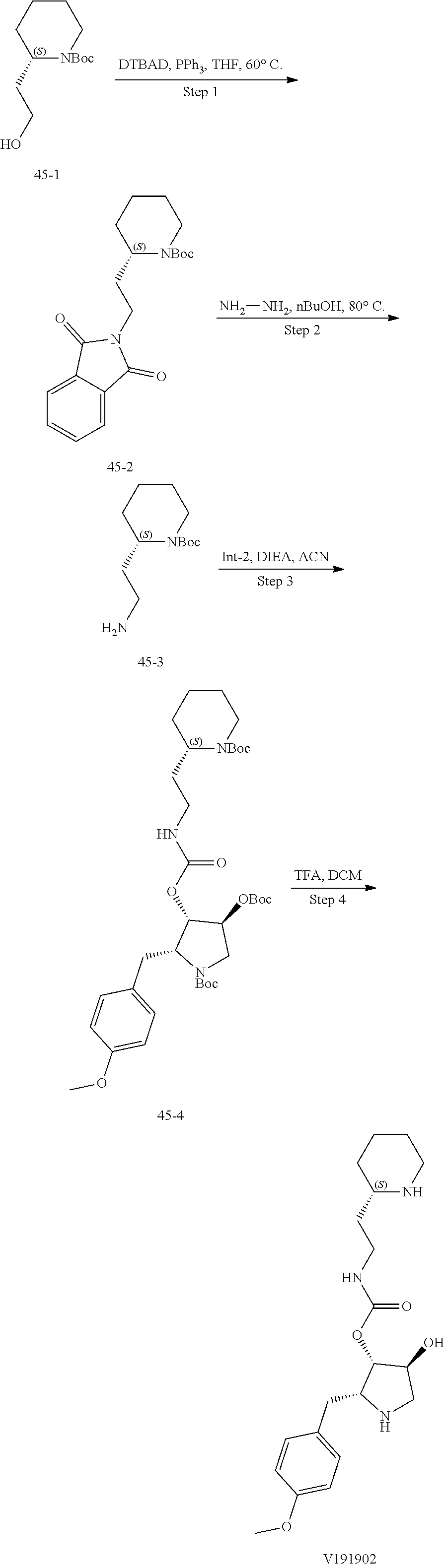

Step 1: To the solution of tert-butyl (2S)-2-(2-hydroxyethyl)piperidine-1-carboxylate (240 mg, 1.05 mmol, 1 eq.) and phthalimide (154 mg, 1.05 mmol, 1 eq.) in THF (5 mL) was added DTBAD (265 mg, 1.15 mmol, 1.1 eq.) and PPh₃ (302 mg, 1.15 mmol, 1.1 eq.). The mixture was stirred at 60° C. under N₂ for overnight. The mixture was concentrated to give the crude product which was purified by Prep TLC (PE:EA=2:1) to afford tert-butyl (2S)-2-[2-(1,3-dioxoisoindol-2-yl)ethyl]piperidine-1-carboxylate (300 mg, 80.0%) as a colorless oil. MS: m/z: Calc'd for C₂₀H₂₆N₂O₄ [M+H]⁺359. found 359.

Step 2: To the solution of tert-butyl (2S)-2-[2-(1,3-dioxoisoindol-2-yl)ethyl]piperidine-1-carboxylate (300 mg, 0.84 mmol, 1 eq.) in n-BuOH (5 mL) was added NH₂NH₂·H₂O (210 mg, 4.19 mmol, 5 eq.). The mixture was stirred at 80° C. for 1 hour. The mixture was concentrated to give the crude product which was purified by Prep HPLC to afford tert-butyl (2S)-2-(2-aminoethyl)piperidine-1-carboxylate (100 mg, 52.3%) as a colorless oil. MS: m/z: Calc'd for C₁₂H₂₄N₂O₂ [M+H−56]⁺229, found 229.

Step 3 and 4: The title compound was prepared in 37.9% overall yield as a white solid according to Substitution reaction for synthesis of Carbamate; General Procedure I using tert-butyl (2S)-2-(2-aminoethyl)piperidine-1-carboxylate (45-3) in STEP 3; Boc Deprotection; General Procedure II in STEP 4. MS: m/z Calc'd for C₂₀H₂₁N₃O₄ [M+H]⁺378. found 378. ¹H NMR (400 MHz, Methanol-d₄) δ 7.29-7.20 (m, 2H), 6.97-6.89 (m, 2H), 4.95 (d, J=3.5 Hz, 1H), 4.39 (d, J=4.1 Hz, 1H), 4.18-4.16 (m, 1H), 3.80 (d, J=1.7 Hz, 3H), 3.63-3.54 (m, 1H), 3.42-3.33 (m, 2H), 3.36-3.23 (m, 1H), 3.22 (d, J=12.7 Hz, 1H), 3.11 (dd, J=14.1, 7.2 Hz, 2H), 3.05-2.89 (m, 2H), 2.12-2.03 (m, 1H), 1.86-1.83 (m, 4H), 1.76-1.41 (m, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 19% B in 10 min; Wave Length: 254 nm/220 nm; RT1(min): 8.72.

### Synthetic Procedure of (2R,3S,4S)-4-hydroxy-2-[(4-methoxyphenyl)methyl]pyrrolidin-3-yl N-[(3-chlorophenyl)methyl]carbamate (V191904)

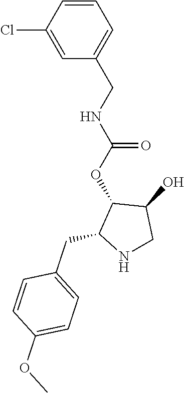 [SMILES: COc1ccc(C[C@H]2NC[C@H](O)[C@H]2OC(=O)NCc2cccc(Cl)c2)cc1]

The title compound was prepared in 38.2% overall yield as a white solid according to Substitution reaction for synthesis of Carbamate; General Procedure I using 1-(3-chlorophenyl)methanamine in STEP 1; Boc Deprotection; General Procedure II in STEP 2. MS: m/z Calc'd for C₂₀H₂₃ClN₂O₄[M+H]⁺391. Found, 391. ¹H NMR (400 MHz, Methanol-d₄) δ 7.41-7.25 (m, 4H), 7.23-7.17 (m, 2H), 6.93-6.85 (m, 2H), 4.93 (d, J=3.6 Hz, 1H), 4.43-4.29 (m, 3H), 4.16-4.14 (m, 1H), 3.79 (s, 3H), 3.58 (dd, J=12.6, 4.3 Hz, 1H), 3.22 (d, J=12.6 Hz, 1H), 3.11 (dd, J=14.0, 7.7 Hz, 1H), 2.97 (dd, J=14.0, 8.1 Hz, 1H).

Prep-HPLC purification conditions: Column: Xselect CSH Prep C18 Column, 30*150 mm, 5 μm; Mobile Phase A: Water (0.1% FA), Mobile Phase B: ACN; Flow rate: 60 m/min; Gradient: 12% B to 42% B in 10 min; Wave Length: 254 nm/220 nm; RT1(min): 10.03.

### Synthetic Procedure of (2R,3S,4S)-4-hydroxy-2-[(4-methoxyphenyl)methyl]pyrrolidin-3-yl N-[(2-cyanophenyl)methyl]carbamate (V191911)

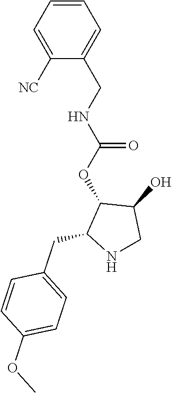 [SMILES: COc1ccc(C[C@H]2NC[C@H](O)[C@H]2OC(=O)NCc2ccccc2C#N)cc1]

The title compound was prepared in 36.3% overall yield as a white solid according to Substitution reaction for synthesis of Carbamate; General Procedure I using 2-(aminomethyl)benzonitrile in STEP 1; Boc Deprotection; General Procedure II in STEP 2. MS: m/z Calc'd for C₂₁H₂₃N₃O₄[M+H]⁺382. Found, 382. ¹H NMR (400 MHz, Methanol-d₄) δ 8.31 (d, J=8.0 Hz, 1H), 7.99 (t, J=7.6 Hz, 1H), 7.86-7.73 (m, 2H), 7.33 (d, J=8.4 Hz, 2H), 6.96 (d, J=8.2 Hz, 2H), 5.35 (dd, J=12.7, 3.9 Hz, 3H), 4.65 (d, J=4.0 Hz, 1H), 4.43-4.35 (m, 1H), 3.80 (s, 3H), 3.69 (s, 1H), 3.53-3.35 (m, 1H), 3.28-3.21 (m, 1H), 3.15-3.06 (m, 1H).

Prep-HPLC purification conditions: Column: Xselect CSH Prep C18 Column, 30*150 mm, 5 μm; Mobile Phase A: Water (0.1% FA), Mobile Phase B: ACN; Flow rate: 60 m/min; Gradient: 40% B to 70% B in 10 min; Wave Length: 254 nm/220 nm; RT1(min): 9.28.

### Synthetic Procedure of (2R,3S,4S)-4-hydroxy-2-[(4-methoxyphenyl)methyl]pyrrolidin-3-yl N-{[3-(difluoromethyl)phenyl]methyl}carbamate (V191954)

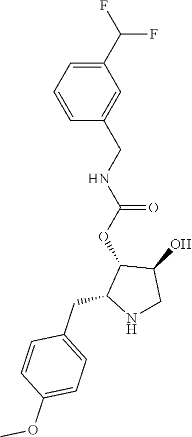 [SMILES: COc1ccc(C[C@H]2NC[C@H](O)[C@H]2OC(=O)NCc2cccc(C(F)F)c2)cc1]

The title compound was prepared in 38.7% overall yield as a white solid according to Substitution reaction for synthesis of Carbamate; General Procedure I using 1-[3-(difluoromethyl)phenyl]methanamine in STEP 1; Boc Deprotection; General Procedure II in STEP 2. MS: m/z Calc'd for C₂₁H₂₄F₂N₂O₄[M+H]⁺407. Found, 407. ¹H NMR (400 MHz, Methanol-d₄) δ 7.53-7.46 (m, 4H), 7.19 (dd, J=8.7, 2.6 Hz, 2H), 6.95-6.78 (m, 3H), 4.95 (d, J=3.4 Hz, 1H), 4.49-4.32 (m, 3H), 4.16-4.14 (m, 1H), 3.78 (d, J=2.6 Hz, 3H), 3.58 (dd, J=12.6, 3.4 Hz, 1H), 3.32 (d, J=1.7 Hz, 1H), 3.16-3.05 (m, 1H), 3.02-2.91 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 11% B to 41% B in 10 min; Wave Length: 254 nm/220 nm; RT1(min): 8.72.

### Synthetic Procedure of (2R,3S,4S)-4-hydroxy-2-[(4-methoxyphenyl)methyl]pyrrolidin-3-yl N-[(2-chlorophenyl)methyl]carbamate (V191915)

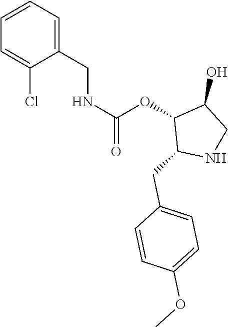 [SMILES: COc1ccc(C[C@H]2NC[C@H](O)[C@H]2OC(=O)NCc2ccccc2Cl)cc1]

The title compound was prepared in 39.4% overall yield as a light brown solid according to Substitution reaction for synthesis of Carbamate; General Procedure I using 1-(2-chlorophenyl)methanamine in STEP 1; Boc Deprotection; General Procedure II in STEP 2. MS: m/z Calc'd for C₂₀H₂₃ClN₂O₄[M+H]⁺391. found 391. ¹H NMR (400 MHz, Methanol-d₄) δ 7.50-7.40 (m, 2H), 7.39-7.27 (m, 2H), 7.25-7.28-7.18 (m, 2H), 6.93-6.85 (m, 2H), 4.94 (d, J=3.5 Hz, 1H), 4.54-4.37 (m, 3H), 4.21-4.11 (m, 1H), 3.79 (s, 3H), 3.59 (dd, J=12.6, 4.3 Hz, 1H), 3.23 (d, J=12.6 Hz, 1H), 3.12 (dd, J=14.1, 7.6 Hz, 1H), 2.99 (dd, J=14.1, 8.2 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 11% B to 41% B in 10 min; Wave Length: 254 nm/220 nm; RT1(min): 9.08.

### Synthetic Procedure of (2R,3S,4S)-4-hydroxy-2-[(4-methoxyphenyl)methyl]pyrrolidin-3-yl N-[2-(oxan-4-yl)ethyl]carbamate (V191930)

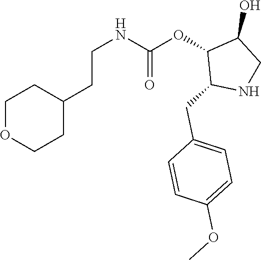 [SMILES: COc1ccc(C[C@H]2NC[C@H](O)[C@H]2OC(=O)NCCC2CCOCC2)cc1]

The title compound was prepared in 29.1% overall yield as a white solid according to Substitution reaction for synthesis of Carbamate; General Procedure I using 2-(oxan-4-yl)ethanamine in STEP 1; Boc Deprotection; General Procedure II in STEP 2. MS: m/z Calc'd for C₂₀H₃₀N₂O₅ [M+H]⁺379. found 379. ¹H NMR (400 MHz, Methanol-d₄) δ 7.28-7.20 (m, 2H), 6.97-6.89 (m, 2H), 4.97-4.92 (m, 1H), 4.38 (d, J=4.2 Hz, 1H), 4.25-4.05 (m, 1H), 3.99-3.90 (m, 2H), 3.80 (s, 3H), 3.57 (dd, J=12.6, 4.4 Hz, 1H), 3.49-3.39 (m, 2H), 3.32-3.18 (m, 3H), 3.12 (dd, J=14.1, 7.3 Hz, 1H), 2.97 (dd, J=14.1, 8.4 Hz, 1H), 1.74-1.59 (m, 3H), 1.58-1.45 (m, 2H), 1.38-1.24 (m, 2H).

Prep-HPLC purification conditions: Column: Xselect CSH Prep Cis Column, 30*150 mm, 5 μm; Mobile Phase A: Water (0.05% TFA), Mobile Phase B: ACN; Flow rate: 60 mL/min; Gradient: 2% B to 32% B in 10 min; Wave Length: 254 nm/220 nm; RT1(min): 8.87.

### Synthetic Procedure of (2R,3S,4S)-4-hydroxy-2-[(4-methoxyphenyl)methyl]pyrrolidin-3-yl N-{2-[(2S)-1-methylpyrrolidin-2-yl]ethyl}carbamate (V191930)

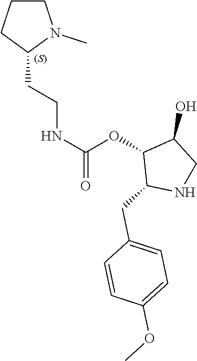

The title compound was prepared in 33.6% overall yield as a white solid according to Substitution reaction for synthesis of Carbamate; General Procedure I using 2-[(2S)-1-methylpyrrolidin-2-yl]ethanamine in STEP 1; Boc Deprotection; General Procedure II in STEP 2. MS: m/z Calc'd for C₂₀H₃₁N₃O₄ [M+H]⁺378. found 378. ¹H NMR (400 MHz, Methanol-d₄) δ 7.28-7.20 (m, 2H), 6.93 (d, J=8.3 Hz, 2H), 4.99 (s, 1H), 4.39 (d, J=4.2 Hz, 1H), 4.18 (d, J=8.7 Hz, 1H), 3.80 (s, 3H), 3.70 (s, 1H), 3.59 (dd, J=12.3, 4.1 Hz, 1H), 3.32-3.07 (m, 6H), 3.02-2.93 (m, 4H), 2.49-2.41 (m, 1H), 1.99-2.25 (m, 3H), 1.82 (t, J=11.3 Hz, 2H).

Prep-HPLC purification conditions: Column: Xselect CSH Prep C₁₈ Column, 30*150 mm, 5 μm; Mobile Phase A: Water (0.05% TFA), Mobile Phase B: ACN; Flow rate: 60 mL/min; Gradient: 2% B to 18% B in 10 min; Wave Length: 254 nm/220 nm; RT1(min): 9.67.

### Synthetic Procedure of (2R,3S,4S)-4-hydroxy-2-[(4-methoxyphenyl)methyl]pyrrolidin-3-yl N-{2-[(2R)-4,4-difluoropyrrolidin-2-yl]ethyl}carbamate (V190526)

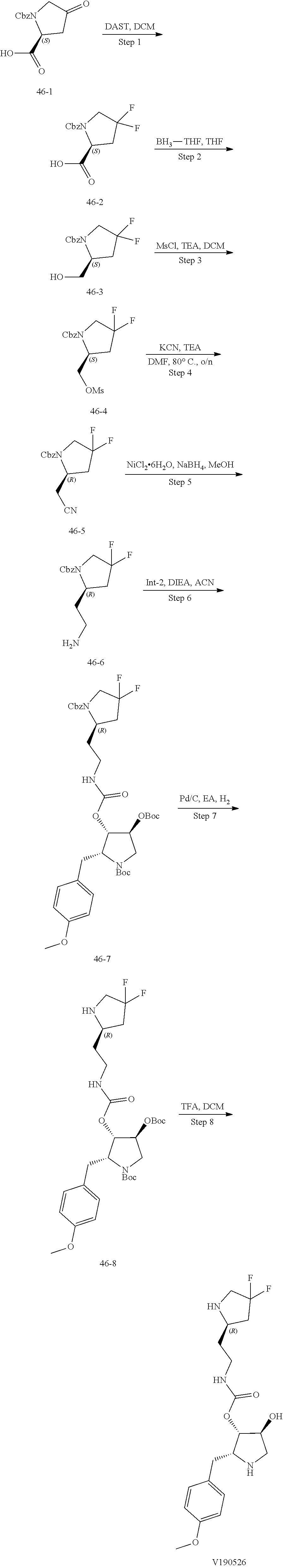

Step 1: To a stirred solution of (2S)-1-[(benzyloxy)carbonyl]-4-oxopyrrolidine-2-carboxylic acid (46-1, 2 g, 7.59 mmol, 1 eq.) in DCM (20 mL) was added DAST (12246.2 mg, 75.97 mmol, 10 eq.) at 0° C. The resulting mixture was stirred at room temperature for overnight. The resulting mixture was concentrated under reduced pressure. The resulting mixture was extracted with CH₂Cl₂ (2×400 mL), dried over anhydrous Na₂SO₄. After filtration, the filtrate was concentrated under reduced pressure. The crude product was purified by reverse phase flash with the following conditions (FA/H₂O) to afford (2S)-1-[(benzyloxy)carbonyl]-4,4-difluoropyrrolidine-2-carboxylic acid (1.8 g, 83.0%) as a dark yellow oil. MS: m/z: Calc'd for C₁₃H1₃F₂NO₄[M+H]⁺286. Found, 286.

Step 2: To a stirred solution of (2S)-1-[(benzyloxy)carbonyl]-4,4-difluoropyrrolidine-2-carboxylic acid (46-2, 1.8 g, 6.31 mmol, 1 eq.) in THF (5 mL) were added BH₃-THF (10 mL) at 0° C. The resulting mixture was stirred at room temperature for overnight. The resulting mixture was concentrated under reduced pressure. The crude product was purified by reversed phase flash to afford benzyl (2S)-4,4-difluoro-2-(hydroxymethyl)pyrrolidine-1-carboxylate (1.36 g, 79.4%) as a light yellow solid. MS: m/z: Calc'd for C₁₃H₁₅F₂NO₃ [M+H]⁺272. Found, 272.

Step 3: To a stirred solution of benzyl (2S)-4,4-difluoro-2-(hydroxymethyl)pyrrolidine-1-carboxylate (46-3, 1.3 g, 5.01 mmol, 1 eq.) in DCM (15 mL) were added TEA (1522.0 mg, 15.04 mmol, 3 eq.) and MSCL (861.3 mg, 7.52 mmol, 1.5 eq.) at 0° C. The resulting mixture was stirred at 0° C. for 1 h. The resulting mixture was concentrated under reduced pressure. The crude product was purified by reverse phase flash with the following conditions (FA/H₂O) to afford benzyl (2S)-4,4-difluoro-2-[(methanesulfonyloxy)methyl]pyrrolidine-1-carboxylate (1.6 g, 91.3%) as a light yellow oil. MS: m/z: Calc'd for C₁₄H₁₇F₂NO₅S [M+H+22]⁺372. Found, 372.

Step 4: To a stirred solution of benzyl (2S)-4,4-difluoro-2-[(methanesulfonyloxy)methyl]pyrrolidine-1-carboxylate (46-4, 1.6 g, 4.58 mmol, 1 eq.) in DMSO (20 mL) were added TEA (1390.3 mg, 13.74 mmol, 3 eq.) and KCN (596.4 mg, 9.16 mmol, 2 eq.) at 0° C. The resulting mixture was stirred at 80° C. for overnight. The resulting mixture was extracted with CH₂Cl₂ (2×200 mL). The combined organic layers were washed with brine (1×200 mL), dried over anhydrous Na₂SO₄. After filtration, the filtrate was concentrated under reduced pressure. The crude product was purified by reversed phase flash to afford benzyl (2R)-2-(cyanomethyl)-4,4-difluoropyrrolidine-1-carboxylate (830 mg, 64.6%) as a light yellow oil. MS: m/z: Calc'd for C₁₄H₁₄F₂N₂O₂[M+H]⁺281. Found, 281.

Step 5: To a stirred solution of benzyl (2R)-2-(cyanomethyl)-4,4-difluoropyrrolidine-1-carboxylate (46-5, 400 mg, 1.42 mmol, 1 eq.) in MeOH (5 mL) were added NiCl₂·6H₂O (678.4 mg, 2.85 mmol, 2 eq.) and NaBH4 (161.9 mg, 4.28 mmol, 3 eq.) at 0° C. The resulting mixture was stirred at room temperature for overnight. The resulting mixture was extracted with EtOAc (2×300 mL). The combined organic layers were washed with brine (1×200 mL), dried over anhydrous Na₂SO₄. After filtration, the filtrate was concentrated under reduced pressure. The crude product (200 mg) was purified by Prep-HPLC to afford benzyl (2R)-2-(2-aminoethyl)-4,4-difluoropyrrolidine-1-carboxylate (170 mg, 41.9%) as a light yellow oil. MS: m/z: Calc'd for C₁₄H₁₈F₂N₂O₂[M+H]⁺285. Found, 285.

Step 6: The compound was prepared in 87.9% yield as a yellow oil according to Substitution reaction for synthesis of Carbamate; General Procedure I using benzyl (2R)-2-(2-aminoethyl)-4,4-difluoropyrrolidine-1-carboxylate (46-6). MS: m/z: Calc'd for C₃₇H₄₉F₂N₃O₁₀[M+H−100−56]⁺578. Found, 578.

Step 7: To a stirred solution of tert-butyl (2R,3S,4S)-3-[({2-[(2R)-1-[(benzyloxy)carbonyl]-4,4-difluoropyrrolidin-2-yl]ethyl}carbamoyl)oxy]-4-[(tert-butoxycarbonyl)oxy]-2-[(4-methoxyphenyl)methyl]pyrrolidine-1-carboxylate (46-7, 110 mg, 0.15 mmol, 1 eq.) in EA (3 mL) was added Pd/C (110.0 mg, 1.03 mmol, 6.90 eq.) at room temperature. The resulting mixture was stirred at room temperature under hydrogen atmosphere for 2 h. The resulting mixture was concentrated under reduced pressure to afford crude product as a light yellow oil. MS: m/z: Calc'd for C₂₉H₄₃F₂N₃O₈[M+H−100−56]⁺600. Found, 600.

Step 8: The title compound was prepared in 28.6% yield as a white solid according to Boc Deprotection; General Procedure II. MS: m/z Calc'd for Calc'd for C₁₉H₂₇F₂N₃O₄[M+H]⁺400. Found, 400. ¹H NMR (400 MHz, Methanol-d₄) δ 7.28-7.20 (m, 2H), 6.98-6.89 (m, 2H), 5.00-4.95 (m, 1H), 4.43-4.37 (m, 1H), 4.18-4.16 (m, 1H), 3.96-3.94 (m, 1H), 3.93-3.80 (m, 4H), 3.71-3.69 (m, 1H), 3.58 (dd, J=12.6, 4.3 Hz, 1H), 3.30-3.22 (m, 2H), 3.12 (dd, J=14.2, 7.0 Hz, 1H), 2.98 (dd, J=14.2, 8.6 Hz, 1H), 2.87-2.85 (m, 1H), 2.76-2.54 (m, 1H), 2.38 (dd, J=18.5, 10.7 Hz, 1H), 2.07 (dd, J=21.0, 14.0 Hz, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 24% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-[(1R)-1-phenylethyl]carbamate (V191970)

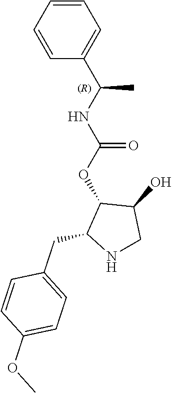

The title compound was prepared in 34.1% overall yield as a white solid according to Substitution reaction for synthesis of Carbamate; General Procedure I using D-a-methylbenzylamine in STEP 1; Boc Deprotection; General Procedure II in STEP 2. MS: m/z Calc'd for C₂₁H₂₆N₂O₄ [M+H]⁺371. found 371. ¹H NMR (400 MHz, Methanol-d₄) δ 7.36-7.26 (d, J=4.3 Hz, 7H), 6.94 (d, J=7.9 Hz, 2H), 6.76 (s, 1H), 4.80 (d, J=7.0 Hz, 1H), 4.32 (s, 1H), 4.16 (s, 1H), 3.81 (s, 3H), 3.55 (dd, J=12.7, 4.3 Hz, 1H), 3.16 (dd, J=27.4, 10.1 Hz, 2H), 3.04-2.94 (m, 1H), 1.53 (d, J=7.0 Hz, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 9% B to 39% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-{2-[(3R)-piperidin-3-yl]ethyl}carbamate (V192046)

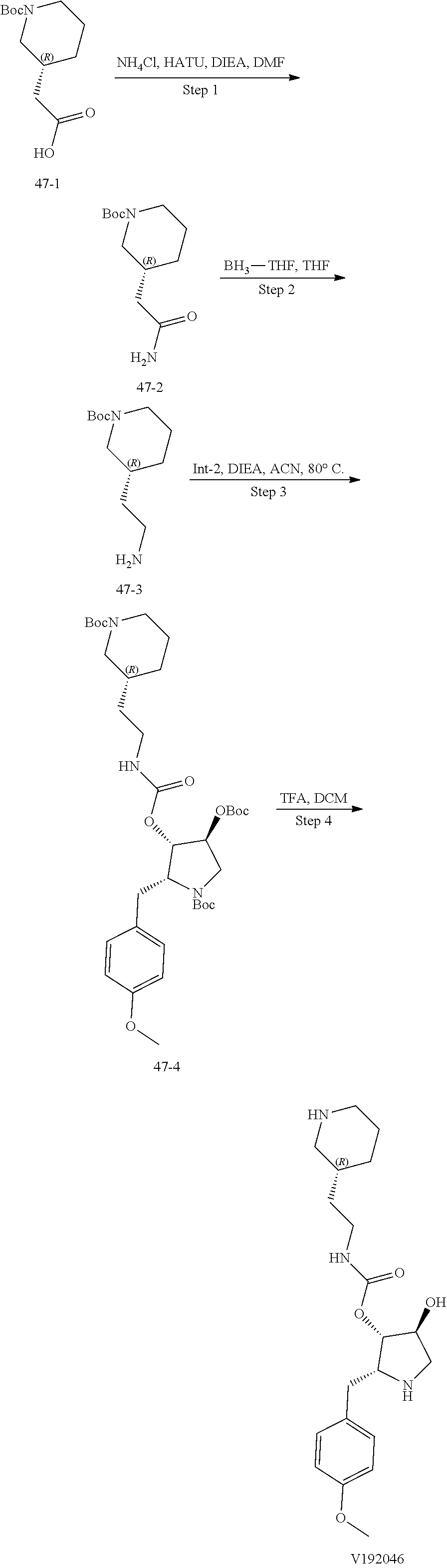

Step 1: To a stirred solution of [(3R)-1-(tert-butoxycarbonyl)piperidin-3-yl]acetic acid (300 mg, 1.23 mmol, 1 eq.) and NH₄Cl (131.9 mg, 2.46 mmol, 2 eq.) in DMF (4 mL) were added HATU (1406.5 mg, 3.69 mmol, 3 eq.) and DIEA (637.4 mg, 4.93 mmol, 4 eq.) at 0° C. The resulting mixture was stirred at room temperature for overnight. The crude product was purified by reverse phase flash with the following conditions (NH₄HCO₃/H₂O) to afford tert-butyl (3R)-3-(carbamoylmethyl)piperidine-1-carboxylate (300 mg, 100%) as a light yellow solid. MS: m/z: Calc'd for C₁₂H₂₂N₂O₃[M+H]⁺243. Found, 243.

Step 2: To a stirred solution of tert-butyl (3R)-3-(carbamoylmethyl)piperidine-1-carboxylate (300 mg, 1.23 mmol, 1 eq.) in THF (1 mL) were added BH₃-THF (3 mL, 31.34 mmol, 25.3 eq.) at 0° C. The resulting mixture was stirred at room temperature for overnight. The resulting mixture was concentrated under reduced pressure to afford tert-butyl (3R)-3-(2-aminoethyl)piperidine-1-carboxylate (250 mg, 88.4%) as a light yellow solid. MS: m/z: Calc'd for C₁₂H₂₄N₂O₂ [M+H]⁺229. Found, 229.

### Step 3 and 4

The title compound was prepared in 26.2% overall yield as a white solid according to Substitution reaction for synthesis of Carbamate; General Procedure I using tert-butyl (3R)-3-(2-aminoethyl)piperidine-1-carboxylate (47-3) in STEP 3; Boc Deprotection; General Procedure II in STEP 4. MS: m/z Calc'd for C₂₀H₂₁N₃O₄ [M+H]⁺378. found 378. ¹H NMR (400 MHz, Methanol-d₄) δ 7.23 (d, J=8.2 Hz, 2H), 6.92 (d, J=8.3 Hz, 2H), 4.96 (d, J=3.5 Hz, 1H), 4.38 (d, J=4.1 Hz, 1H), 4.16-4.14 (m, 1H), 3.79 (s, 3H), 3.57 (dd, J=12.6, 4.3 Hz, 1H), 3.37 (s, 2H), 3.22 (d, J=7.7 Hz, 3H), 3.10 (dd, J=14.2, 7.0 Hz, 1H), 2.94 (d, J=13.8 Hz, 1H), 2.84 (d, J=9.0 Hz, 1H), 2.69 (t, J=12.1 Hz, 1H), 2.02-1.91 (m, 2H), 1.84 (s, 1H), 1.75 (dd, J=10.4, 6.8 Hz, 1H), 1.55 (dd, J=7.0, 3.3 Hz, 2H), 1.30-1.28 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 24% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-[2-(piperidin-4-yl)ethyl]carbamate (V192049)

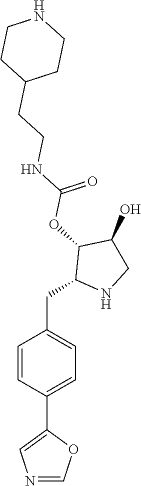 [SMILES: O=C(NCCC1CCNCC1)O[C@@H]1[C@@H](O)CN[C@@H]1Cc1ccc(-c2cnco2)cc1]

The title compound was prepared in 37.2% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 4-(2-aminoethyl)piperidine-1-carboxylate in STEP 1; Boc Deprotection; General Procedure II in STEP 2. MS: m/z Calc'd for C₂₂H₃₀N₄O₄[M+H]⁺415. found 415. ¹H NMR (400 MHz, Methanol-d₄) δ 8.26 (s, 1H), 7.75 (d, J=8.2 Hz, 1H), 7.65 (d, J=8.6 Hz, 1H), 7.53 (s, 1H), 7.48-7.45 (m, 1H), 7.44-7.41 (m, 1H), 5.02-4.96 (m, 1H), 4.39 (d, J=4.1 Hz, 1H), 4.25-4.23 (m, 1H), 3.67-3.55 (m, 1H), 3.40 (d, J=12.7 Hz, 1H), 3.35 (d, J=8.4 Hz, 1H), 3.30-3.16 (m, 4H), 3.10 (dd, J=14.2, 8.4 Hz, 1H), 2.98-2.96 (m, 2H), 2.05-1.97 (m, 2H), 1.56 (d, J=7.2 Hz, 1H), 1.46 (d, J=14.7 Hz, 2H), 1.41 (s, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 15% B in 15 min; Wave Length: 254 nm/220 nm; RT1(min): 8.6.

### Synthetic Procedure of (2R,3S,4S)-4-hydroxy-2-[(4-methoxyphenyl)methyl]pyrrolidin-3-yl N-{[2-(3,3-difluoroazetidin-1-yl)pyridin-4-yl]methyl}carbamate (V192066)

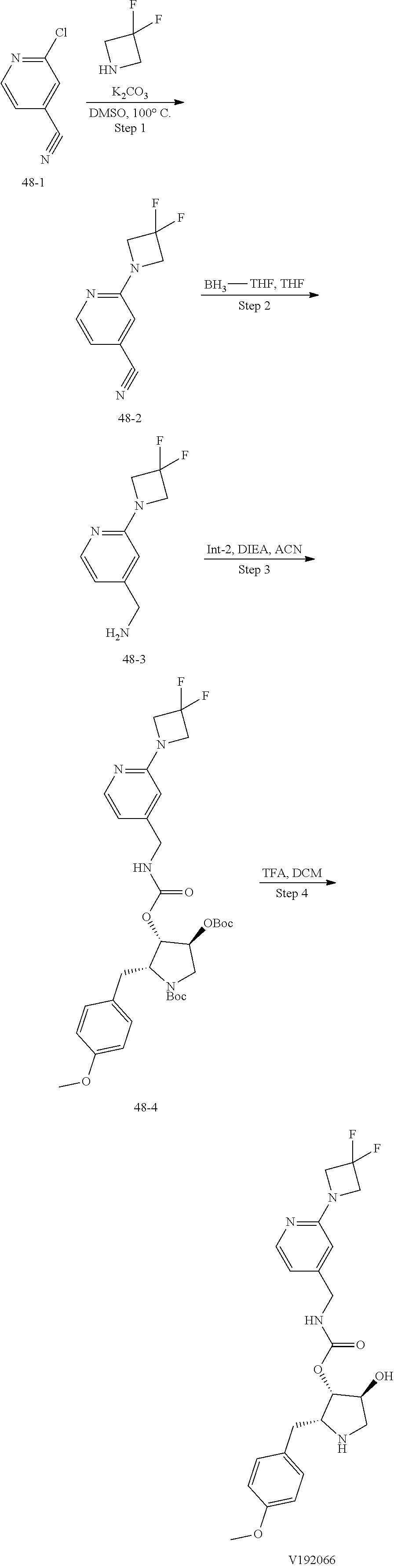

Step 1: To a stirred solution of 2-chloropyridine-4-carbonitrile (48-1, 300 mg, 2.16 mmol, 1 eq.) and 3,3-difluoroazetidine (403.0 mg, 4.33 mmol, 2 eq.) in DMSO (5 mL) was added K₂CO₃ (897.7 mg, 6.49 mmol, 3 eq.) at 0° C. The resulting mixture was stirred at room temperature for overnight. The crude product was purified by reversed phase flash to afford 2-(3,3-difluoroazetidin-1-yl)pyridine-4-carbonitrile (300 mg, 70.9%) as a light yellow semi-solid. MS: m/z: Calc'd for C₉H₇F₂N₃[M+H]⁺196. Found, 196.

Step 2: To a stirred solution of 2-(3,3-difluoroazetidin-1-yl)pyridine-4-carbonitrile (48-2, 295 mg, 1.51 mmol, 1 eq.) in THF (1 mL) were added BH₃-THF (3 mL) at 0° C. The resulting mixture was stirred at room temperature for overnight. The resulting mixture was concentrated under reduced pressure to afford tert-butyl (3R)-3-(2-aminoethyl)piperidine-1-carboxylate (250 mg, 88.4%) as a light yellow solid. MS: m/z: Calc'd for C₉H₁₁F₂N₃ [M+H]⁺200. Found, 200.

Step 3 and 4: The title compound was prepared in 34.7% overall yield as an off-white solid according to Substitution reaction for synthesis of Carbamate; General Procedure I using 1-[2-(3,3-difluoroazetidin-1-yl)pyridin-4-yl]methanamine (83-3) in STEP 3; Boc Deprotection; General Procedure II in STEP 4. MS: m/z Calc'd for C₂₀H₃₁N₃O₄[M+H]⁺378. Found, 378. ¹H NMR (400 MHz, Methanol-d₄) δ 8.04 (d, J=6.0 Hz, 1H), 7.23 (d, J=8.5 Hz, 2H), 6.96-6.86 (m, 3H), 6.72 (s, 1H), 4.99 (d, J=3.6 Hz, 1H), 4.57 (t, J=11.8 Hz, 4H), 4.46-4.35 (m, 3H), 4.19-4.17 (m, 1H), 3.79 (s, 3H), 3.68-3.56 (m, 1H), 3.24 (d, J=12.6 Hz, 1H), 3.13 (dd, J=14.0, 7.1 Hz, 1H), 3.00 (dd, J=14.1, 8.4 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 26% B in 10 min; Wave Length: 254 nm/220 nm; RT1(min): 9.1.

### Synthetic Procedure of (2R,3S,4S)-4-hydroxy-2-[(4-methoxyphenyl)methyl]pyrrolidin-3-yl N-{[2-(azetidin-1-yl)pyrimidin-4-yl]methyl}carbamate (V192073)

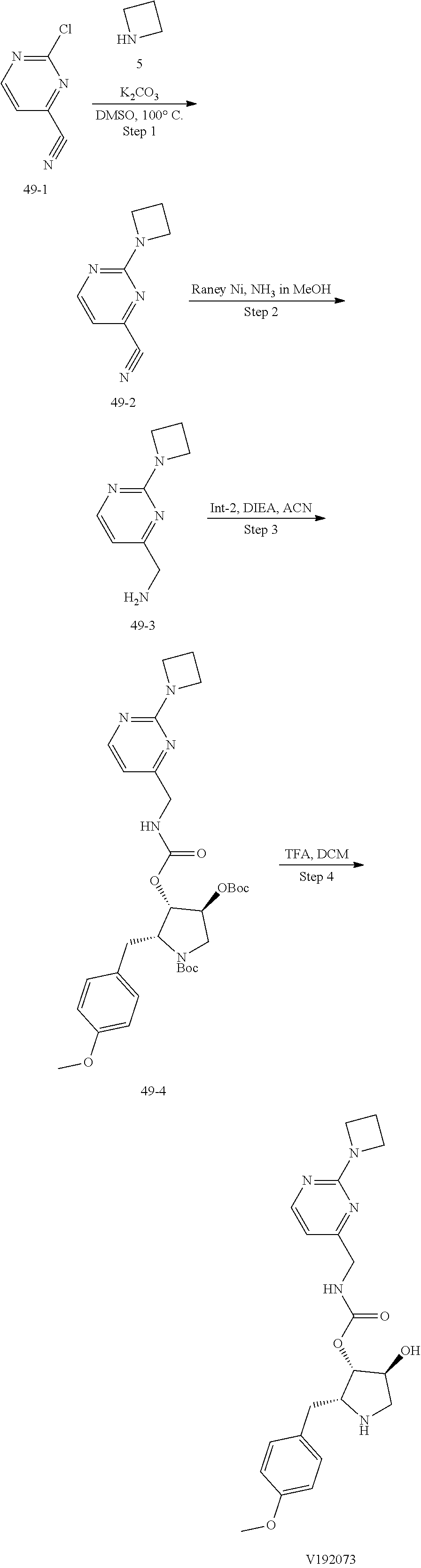

Step 1: To a stirred solution of 2-chloropyrimidine-4-carbonitrile (300 mg, 2.15 mmol, 1 eq.) and azetidine (245.5 mg, 4.30 mmol, 2 eq.) in DMSO (4 mL) was added K₂CO₃ (891.3 mg, 6.45 mmol, 3 eq.) at 0° C. The resulting mixture was stirred at room temperature for overnight. The crude product was purified by reversed phase flash to afford 2-(azetidin-1-yl)pyrimidine-4-carbonitrile (95 mg, 27.5%) as a light yellow semi-solid. MS: m/z: Calc'd for C₈H₈N₄ [M+H]⁺161. Found, 161.

Step 2: To a stirred solution of 2-(azetidin-1-yl)pyrimidine-4-carbonitrile (70 mg, 0.43 mmol, 1 eq.) in NH₃(g) in MeOH (3 mL) were added RaneyNi (30 mg) at room temperature. The resulting mixture was stirred at room temperature for 30 min. The resulting mixture was filtered, the filter cake was washed with MeOH (1×10 mL). The filtrate was concentrated under reduced pressure to afford 1-[2-(azetidin-1-yl)pyrimidin-4-yl]methanamine (70 mg, 97.5%) as an off-white solid. MS: m/z: Calc'd for C₈H₁₂N₄[M+H]⁺165. Found, 165.

### Step 3 and 4

The title compound was prepared in 26.9% overall yield as an off-white solid according to Substitution reaction for synthesis of Carbamate; General Procedure I using 1-[2-(azetidin-1-yl)pyrimidin-4-yl]methanamine (49-3) in STEP 3; Boc Deprotection; General Procedure II in STEP 4. MS: m/z Calc'd for C₂₁H₂₇N504[M+H]⁺414. Found, 414. ¹H NMR (400 MHz, Methanol-d₄) δ 8.25 (d, J=5.7 Hz, 1H), 7.28-7.20 (m, 2H), 6.95-6.87 (m, 2H), 6.76 (dd, J=8.5, 3.9 Hz, 1H), 4.98 (d, J=3.6 Hz, 1H), 4.44-4.33 (m, 3H), 4.31-4.18 (m, 5H), 3.79 (s, 3H), 3.62 (dd, J=13.0, 4.3 Hz, 1H), 3.24 (d, J=12.6 Hz, 1H), 3.14 (dd, J=14.1, 7.3 Hz, 1H), 3.02 (dd, J=14.1, 8.5 Hz, 1H), 2.46 (d, J=7.5 Hz, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 27% B in 10 min; Wave Length: 254 nm/220 nm; RT1(min): 9.1.

### Synthetic Procedure of (2R,3S,4S)-2-{[4-(1,3-benzothiazol-6-yl)phenyl]methyl}-4-hydroxypyrrolidin-3-yl N-[(3-fluorophenyl)methyl]carbamate (V192103)

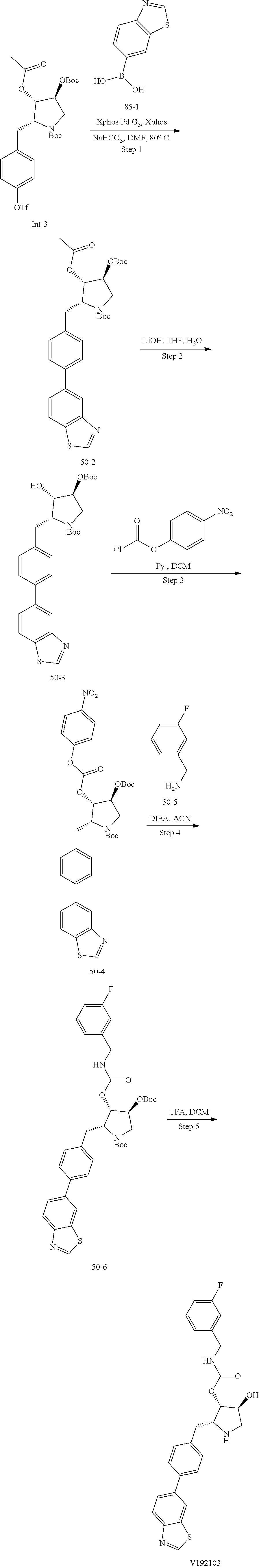

Step 1: To a stirred solution of tert-butyl (2R,3S,4S)-3-(acetyloxy)-4-hydroxy-2-{[4-(trifluoromethanesulfonyloxy)phenyl]methyl}pyrrolidine-1-carboxylate (Int-3, 150 mg, 0.31 mmol, 1 eq.) and 1,3-benzothiazol-6-ylboronic acid (50-1, 92.0 mg, 0.51 mmol, 1.2 eq.) in DMF (4 mL) were added XPhos Pd G₃ (36.2 mg, 0.04 mmol, 0.1 eq.), X-Phos (40.8 mg, 0.08 mmol, 0.2 eq.) and NaHCO₃ (52.1 mg, 0.62 mmol, 2 eq.) at room temperature. The reaction was placed under vacuum, sonicated and backfilled with nitrogen. The resulting mixture was stirred at 80° C. for overnight. After completion of reaction monitored by LCMS. The reaction mixture was quenched with water and extracted with EA. The extracts were washed with brine, dried over anhydrous sodium sulfate, filtered and concentrated. The residue was purified by reversed-phase flash to obtain tert-butyl (2R,3S,4S)-3-(acetyloxy)-2-{[4-(1,3-benzothiazol-5-yl)phenyl]methyl}-4-[(tert-butoxycarbonyl)oxy]pyrrolidine-1-carboxylate (195 mg, 80.0%) as an off-white solid. MS: m/z: Calc'd for C₃₀H₃₆N₂O₇S [M+H]⁺569. Found, 569.

Step 2: To a stirred solution of tert-butyl (2R,3S,4S)-3-(acetyloxy)-2-{[4-(1,3-benzothiazol-5-yl)phenyl]methyl}-4-[(tert-butoxycarbonyl)oxy]pyrrolidine-1-carboxylate (50-2, 190 mg, 0.33 mmol, 1 eq.) in THF (3 mL) and H₂O (1 mL) were added LiOH (80.0 mg, 3.34 mmol, 10 eq.) at room temperature. The resulting mixture was stirred at room temperature for overnight. The resulting mixture was concentrated under reduced pressure to afford tert-butyl (2R,3S,4S)-2-{[4-(1,3-benzothiazol-5-yl)phenyl]methyl}-4-[(tert-butoxycarbonyl)oxy]-3-hydroxypyrrolidine-1-carboxylate (100 mg, 56.8%) as an off-white solid. MS: m/z: Calc'd for C₂₈H₃₄N₂O₆S [M+H]⁺527. Found, 527.

Step 3: To a stirred solution of tert-butyl (2R,3S,4S)-2-{[4-(1,3-benzothiazol-5-yl)phenyl]methyl}-4-[(tert-butoxycarbonyl)oxy]-3-hydroxypyrrolidine-1-carboxylate (50-3, 100 mg, 0.19 mmol, 1 eq.) and 4-nitrophenyl carbonochloridate (76.5 mg, 0.38 mmol, 2 eq.) in DCM (3 mL) were added Pyridine (45.0 mg, 0.57 mmol, 3 eq.) at 0° C. The resulting mixture was stirred at room temperature for overnight. The resulting mixture was concentrated under reduced pressure. The residue was purified by Prep-TLC (PE/EA 1:1) to afford tert-butyl (2R,3S,4S)-2-{[4-(1,3-benzothiazol-5-yl)phenyl]methyl}-4-[(tert-butoxycarbonyl)oxy]-3-[(4-nitrophenoxycarbonyl)oxy]pyrrolidine-1-carboxylate (43 mg, 32.7%) as a yellow oil. MS: m/z: Calc'd for C₃₅H₃₇N₃O₁₀S [M+H]⁺692. Found, 692.

Step 4 and 5: The title compound was prepared in 59.0% overall yield as an off-white solid according to Substitution reaction for synthesis of Carbamate; General Procedure I using tert-butyl (2R,3S,4S)-2-{[4-(1,3-benzothiazol-5-yl)phenyl]methyl}-4-[(tert-butoxycarbonyl)oxy]-3-[(4-nitrophenoxycarbonyl)oxy]pyrrolidine-1-carboxylate (50-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine (50-6) in STEP 4; Boc Deprotection; General Procedure II in STEP 5. MS: m/z Calc'd for C₂₆H₂₄FN₃O₃S [M+H]⁺478. found 478. ¹H NMR (400 MHz, Methanol-d₄) δ 9.29 (s, 1H), 8.33 (d, J=1.9 Hz, 1H), 8.16 (d, J=8.5 Hz, 1H), 7.84 (dd, J=8.6, 1.8 Hz, 1H), 7.75-7.69 (m, 2H), 7.44 (d, J=7.9 Hz, 2H), 7.39-7.37 (m, 1H), 7.18 (d, J=7.7 Hz, 1H), 7.12 (d, J=9.9 Hz, 1H), 7.04 (t, J=8.7 Hz, 1H), 5.01 (d, J=3.5 Hz, 1H), 4.47-4.41 (m, 1H), 4.38-4.35 (m, 1H), 4.33-4.25 (m, 1H), 3.62 (dd, J=12.7, 4.2 Hz, 1H), 3.31-3.21 (m, 3H), 3.15-3.07 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 9% B to 30% B in 10 min; Wave Length: 254 nm/220 nm; RT1(min): 9.5.

### Synthetic Procedure of (2R,3S,4S)-2-({4-[2-(azetidin-1-yl)-1,3-thiazol-5-yl]phenyl}methyl)-4-hydroxypyrrolidin-3-ylN-[(3-fluorophenyl)methyl]carbamate (V192116)

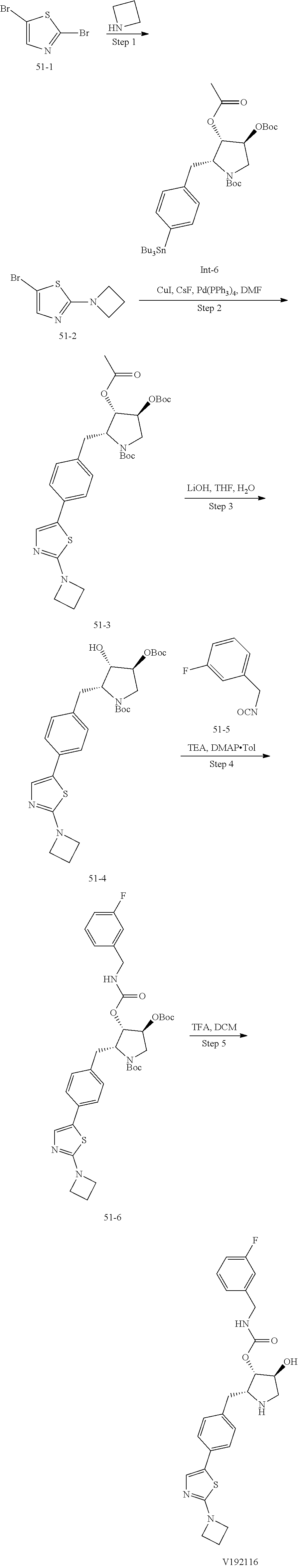

Step 1: To a stirred solution of 2,5-dibromo-1,3-thiazole (500 mg, 2.058 mmol, 1 eq.) and azetidine (235.0 mg, 4.11 mmol, 2 eq.) in DMF (8 mL) were added K₂CO₃ (853.4 mg, 6.17 mmol, 3 eq.) at ° C. The resulting mixture was stirred at 100° C. for overnight. The resulting mixture was extracted with EtOAc (2×100 mL). The combined organic layers were washed with brine (2×40 mL), dried over anhydrous Na₂SO₄. After filtration, the filtrate was concentrated under reduced pressure. The residue was purified by Prep-TLC (PE/EA 3:1) to afford 2-(azetidin-1-yl)-5-bromo-1,3-thiazole (260 mg, 57.6%) as an off-white solid. MS: m/z: Calc'd for C₆H₇BrN₂S [M+H]⁺219. Found, 219.

### Step 2, 3, 4, and 5

The title compound was prepared in 67.8% overall yield as an off-white according to Stille Coupling with Pd(PPh₃)₄, CuI and CsF; General Procedure IV using 2-(azetidin-1-yl)-5-bromo-1,3-thiazole (51-2) in STEP 2; Hydrolysis reaction with LiOH; General Procedure V using tert-butyl (2R,3S,4S)-3-(acetyloxy)-4-[(tert-butoxycarbonyl)oxy]-2-{[4-(tributylstannyl)phenyl]methyl}pyrrolidine-1-carboxylate (51-3) instead of tert-butyl (2R,3S,4S)-3-(acetyloxy)-4-[(tert-butoxycarbonyl)oxy]-2-[(4-fluorophenyl)methyl]pyrrolidine-1-carboxylate (3-1) in STEP 3; Addition reaction: Isocyanates and hydroxyl groups; General Procedure VI using tert-butyl (2R,3S,4S)-2-({4-[2-(azetidin-1-yl)-1,3-thiazol-5-yl]phenyl}methyl)-4-[(tert-butoxycarbonyl)oxy]-3-hydroxypyrrolidine-1-carboxylate (51-4) instead of tert-butyl (2R,3S,4S)-4-[(tert-butoxycarbonyl)oxy]-2-[(4-fluorophenyl)methyl]-3-hydroxypyrrolidine-1-carboxylate (Int-7), 1-fluoro-3-(isocyanatomethyl)benzene STEP 4; Boc Deprotection; General Procedure II in STEP 5. MS: m/z Calc'd for C₂₅H₂₇FN₄O₃S [M+H]⁺483. found 483. ¹H NMR (400 MHz, Methanol-d₄) δ 7.56 (s, 1H), 7.48 (s, 2H), 7.46 (d, J=1.9 Hz, 1H), 7.43-7.35 (m, 2H), 7.33 (d, J=8.1 Hz, 1H), 7.16 (d, J=7.7 Hz, 1H), 7.07 (dd, J=14.6, 9.2 Hz, 1H), 4.94 (d, J=3.6 Hz, 1H), 4.44-4.36 (m, 2H), 4.35-4.19 (m, 6H), 3.60 (dd, J=12.6, 4.3 Hz, 1H), 3.26 (d, J=12.6 Hz, 1H), 3.18 (dd, J=13.9, 7.9 Hz, 1H), 3.08 (dd, J=13.9, 7.9 Hz, 1H), 2.61 (d, J=7.6 Hz, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 6% B to 36% B in 10 min; Wave Length: 254 nm/220 nm; RT1(min): 8.5.

### Synthetic Procedure of (2R,3S,4S)-4-hydroxy-2-[(4-{imidazo[2,1-b][1,3]thiazol-2-yl}phenyl)methyl]pyrrolidin-3-yl N-[(3-fluorophenyl)methyl]carbamate (V192127)

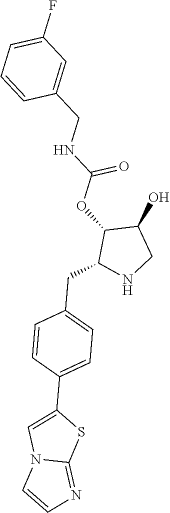 [SMILES: O=C(NCc1cccc(F)c1)O[C@@H]1[C@@H](O)CN[C@@H]1Cc1ccc(-c2cn3ccnc3s2)cc1]

The title compound was prepared in 3.2% overall yield as an off-white solid according to Stille Coupling with Pd(PPh₃)₄, CuI and CsF; General Procedure IV using 2-bromoimidazo[2,1-b][1,3]thiazole in STEP 1; Hydrolysis reaction with LiOH; General Procedure V using tert-butyl (2R,3S,4S)-3-(acetyloxy)-4-[(tert-butoxycarbonyl)oxy]-2-[(4-{imidazo[2,1-b][1,3]thiazol-2-yl}phenyl)methyl]pyrrolidine-1-carboxylate instead of tert-butyl (2R,3S,4S)-3-(acetyloxy)-4-[(tert-butoxycarbonyl)oxy]-2-[(4-fluorophenyl)methyl]pyrrolidine-1-carboxylate (3-1) in STEP 2; Addition reaction: Isocyanates and hydroxyl groups; General Procedure VI using tert-butyl (2R,3S,4S)-4-[(tert-butoxycarbonyl)oxy]-3-hydroxy-2-[(4-{imidazo[2,1-b][1,3]thiazol-2-yl}phenyl)methyl]pyrrolidine-1-carboxylate instead of tert-butyl (2R,3S,4S)-4-[(tert-butoxycarbonyl)oxy]-2-[(4-fluorophenyl)methyl]-3-hydroxypyrrolidine-1-carboxylate (Int-7), (3-fluorophenyl)methyl cyanate in STEP 3; Boc Deprotection; General Procedure II in STEP 4. MS: m/z Calc'd for C₂₄H₂₃FN₄O₃S[M+H]⁺467. Found, 467. ¹H NMR (400 MHz, Methanol-d₄) δ 8.31 (s, 1H), 7.88 (s, 1H), 7.63 (d, J=8.0 Hz, 2H), 7.53 (s, 1H), 7.42 (dd, J=17.0, 7.3 Hz, 2H), 7.37 (d, J=7.4 Hz, 1H), 7.34-7.21 (m, 1H), 7.17 (d, J=7.7 Hz, 1H), 7.14-7.01 (m, 1H), 4.96 (d, J=3.6 Hz, 1H), 4.40 (d, J=15.2 Hz, 2H), 4.36-4.23 (m, 2H), 3.61 (dd, J=12.6, 4.2 Hz, 1H), 3.31-3.25 (m, 1H), 3.21-3.18 (s, 1H), 3.18-3.09 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 15 min; Wave Length: 254 nm/220 nm; RT1(min): 13.5.

### Synthetic Procedure of (2R,3S,4S)-4-hydroxy-2-{[4-(1,3-oxazol-5-yl)phenyl]methyl}pyrrolidin-3-yl N-{[3-(azetidin-3-yl)phenyl]methyl}carbamate (V192154)

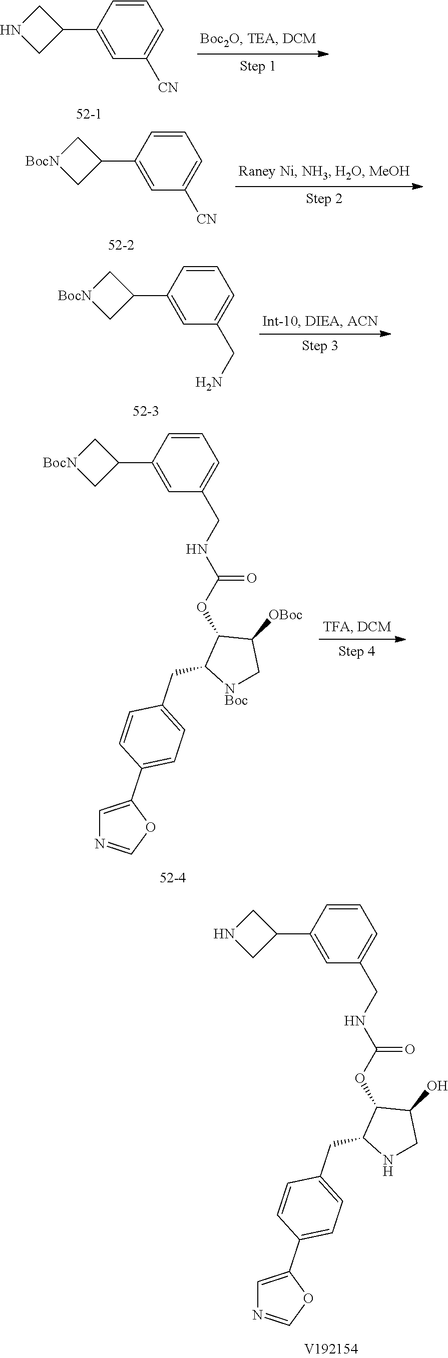

Step 1: To a stirred solution of 3-(azetidin-3-yl)benzonitrile (450 mg, 2.84 mmol, 1 eq.) and TEA (863.5 mg, 8.53 mmol, 3 eq.) in DCM (8 mL) were added Boc₂O (5307.4 mg, 22.75 mmol, 8 eq.) at 0° C. The resulting mixture was stirred at room temperature for overnight. The resulting mixture was concentrated under reduced pressure. The crude product was purified by reverse phase flash with the following conditions (TFA/H₂O) to afford tert-butyl 3-(3-cyanophenyl)azetidine-1-carboxylate (70 mg, 9.5%) as a white solid. MS: m/z: Calc'd for C₁₅H₁₈N₂O₂[M+H−56+41]⁺244. Found, 244.

Step 2: To a stirred solution of tert-butyl 3-(3-cyanophenyl)azetidine-1-carboxylate (65 mg, 0.25 mmol, 1 eq.) in MEOH (3 mL) were added Ammonium hydroxide (9% in water) (0.1 mL, 0.01 mmol, 0.01 eq.) and raney nickel (147.6 mg, 2.52 mmol, 10 eq.) at 0° C. The resulting mixture was stirred at room temperature under hydrogen atmosphere for 2 h. The precipitated solids were collected by filtration and washed with MEOH (3 mL). The filtrate was concentrated under reduced pressure. The crude product (60 mg) was purified by Prep-HPLC to afford tert-butyl 3-[3-(aminomethyl)phenyl]azetidine-1-carboxylate (20 mg, 30.3% yield, 96% purity) as a white solid. MS: m/z: Calc'd for C₁₅H₂₂N₂O₂ [M+H−56]⁺207. Found, 207.

Step 3 and 4: The title compound was prepared in 55.5% overall yield as a yellow solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 3-[3-(aminomethyl)phenyl]azetidine-1-carboxylate (52-3) in STEP 3; Boc Deprotection; General Procedure II in STEP 4. MS: m/z Calc'd for C₂₅H₂₈N₄O₄[M+H]⁺449. Found, 449. ¹H NMR (400 MHz, Methanol-d₄) δ 8.29 (s, 1H), 7.69 (d, J=8.0 Hz, 2H), 7.53 (s, 1H), 7.46 (d, J=7.7 Hz, 3H), 7.44-7.32 (m, 3H), 4.99 (s, 1H), 4.38 (d, J=8.9 Hz, 5H), 4.29-4.26 (dd, J=19.0, 5.4 Hz, 4H), 3.63 (d, J=4.2 Hz, 1H), 3.26 (d, J=12.5 Hz, 2H), 3.20 (d, J=7.4 Hz, 1H).

Prep-HPLC purification conditions: Column: X select CSH Prep C18 Column, 30*250 mm, 10 μm; Mobile Phase A: Water (0.05% TFA), Mobile Phase B: ACN; Flow rate: 60 mL/min; Gradient: 2% B to 15% B in 18 min; Wave Length: 254 nm/220 nm; RT1(min): 17.8.

### Synthetic Procedure of (2R,3S,4S)-4-hydroxy-2-{[4-(1,3-oxazol-5-yl)phenyl]methyl}pyrrolidin-3-yl N-[2-(trifluoromethoxy)ethyl]carbamate (V192265)

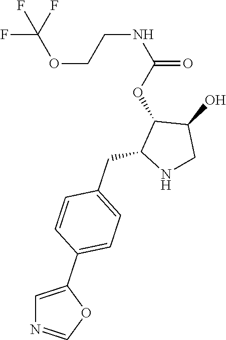 [SMILES: O=C(NCCOC(F)(F)F)O[C@@H]1[C@@H](O)CN[C@@H]1Cc1ccc(-c2cnco2)cc1]

The title compound was prepared in 74.2% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 2-(trifluoromethoxy)ethanamine in STEP 1; Boc Deprotection; General Procedure II in STEP 2. MS: m/z Calc'd for C₁₈H₂₀F₃N₃O₅ [M+H]⁺416. found 416. ¹H NMR (400 MHz, Methanol-d₄) δ 8.27 (s, 1H), 7.76 (dd, J=8.4, 2.3 Hz, 2H), 7.54-7.41 (m, 3H), 5.01 (d, J=3.2 Hz, 1H), 4.42 (d, J=4.2 Hz, 1H), 4.27-4.25 (m, 1H), 4.15 (dd, J=5.8, 4.5 Hz, 2H), 3.63-3.41 (m, 3H), 3.30-3.19 (m, 2H), 3.19-3.04 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 6% B to 26% B in 16 min; Wave Length: 254 nm/220 nm; RT1(min): 15.5.

### Synthetic Procedure of (2R,3S,4S)-4-hydroxy-2-{[4-(1,3-oxazol-5-yl)phenyl]methyl}pyrrolidin-3-yl N-{[5-(azetidin-1-yl)pyridin-3-yl]methyl}carbamate (V192286)

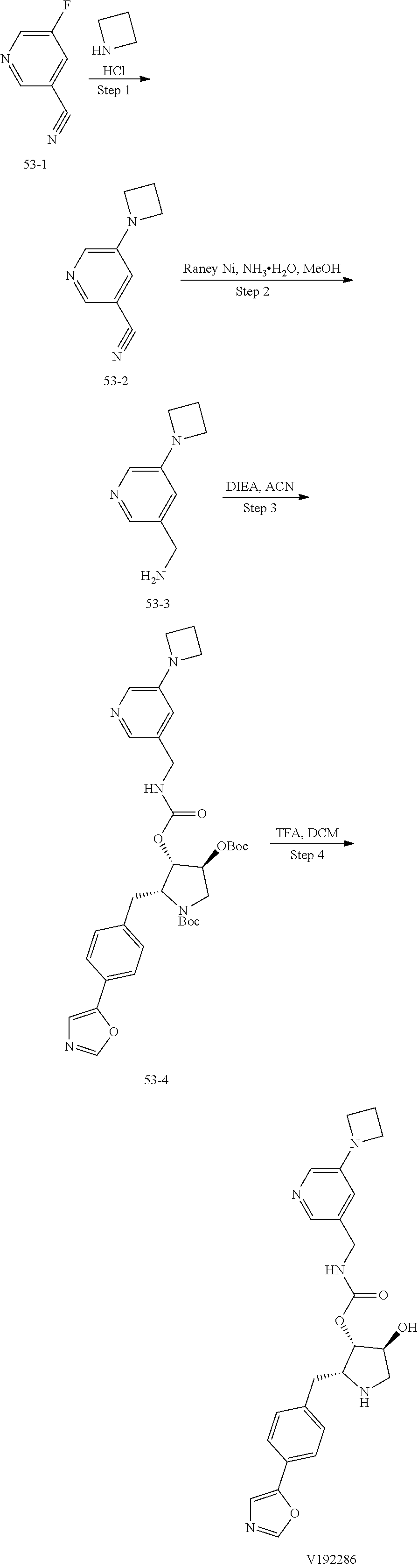

Step 1: To a stirred solution of 5-fluoropyridine-3-carbonitrile (200 mg, 1.63 mmol, 1 eq.) and azetidine hydrochloride (306.4 mg, 3.27 mmol, 2 eq.) in DMF (5 mL) was added K₂CO₃ (679.1 mg, 4.91 mmol, 3 eq) at 80° C. for overnight. The resulting mixture was extracted with EtOAc (2×50 mL). The combined organic layers were washed with brine (2×20 mL), dried over anhydrous Na₂SO₄. After filtration, the filtrate was concentrated under reduced pressure. The crude product was purified by reversed phase flash (NH₄HCO₃/H₂O) to afford 5-(azetidin-1-yl)pyridine-3-carbonitrile (100 mg, 38.3% yield, 98% purity) as a white solid. MS: m/z: Calc'd for C₉H₉N₃ [M+H]⁺160. Found, 160.

Step 2: To a stirred solution of 5-(azetidin-1-yl)pyridine-3-carbonitrile (95 mg, 0.59 mmol, 1 eq.) in methanol (3 mL) were added raney nickel (105.0 mg, 1.79 mmol, 3 eq.) and Ammonium hydroxide (9% in water) (30 mg, 0.85 mmol, 1.4 eq.) at room temperature under hydrogen atmosphere for 2 h. The precipitated solids were collected by filtration and washed with methanol (5 mL) (1×20 mL). The resulting mixture was concentrated under reduced pressure to afford 1-[5-(azetidin-1-yl)pyridin-3-yl]methanamine (80 mg) as a white solid. MS: m/z: Calc'd for C₉H₁₃N3 [M+H]⁺164. Found, 164.

### Step 3 and 4

The title compound was prepared in 20.8% overall yield as a yellow solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-[5-(azetidin-1-yl)pyridin-3-yl]methanamine (53-3) in STEP 3; Boc Deprotection; General Procedure II in STEP 4. MS: m/z Calc'd for C₂₄H₂₇N₅O₄[M+H]⁺450. Found, 450. ¹H NMR (400 MHz, Methanol-d₄) δ 8.29 (s, 1H), 7.98 (s, 1H), 7.82 (s, 1H), 7.72 (d, J=8.0 Hz, 2H), 7.55 (s, 1H), 7.42 (d, J=8.0 Hz, 2H), 7.34 (d, J=2.2 Hz, 1H), 5.04-4.98 (m, 1H), 4.50-4.35 (m, 3H), 4.28-4.26 (m, 1H), 4.10 (t, J=7.4 Hz, 4H), 3.62 (dd, J=12.6, 4.3 Hz, 1H), 3.23 (dd, J=22.2, 9.8 Hz, 2H), 3.10 (dd, J=14.1, 8.5 Hz, 1H), 2.52 (d, J=7.4 Hz, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 15% B in 18 min; Wave Length: 254 nm/220 nm; RT1(min): 16.9.

### Synthetic Procedure of (2R,3S,4S)-4-hydroxy-2-{[4-(1,3-oxazol-5-yl)phenyl]methyl}pyrrolidin-3-yl N-({3-[1-(methylamino)cyclopropyl]phenyl}methyl)carbamate (V192289)

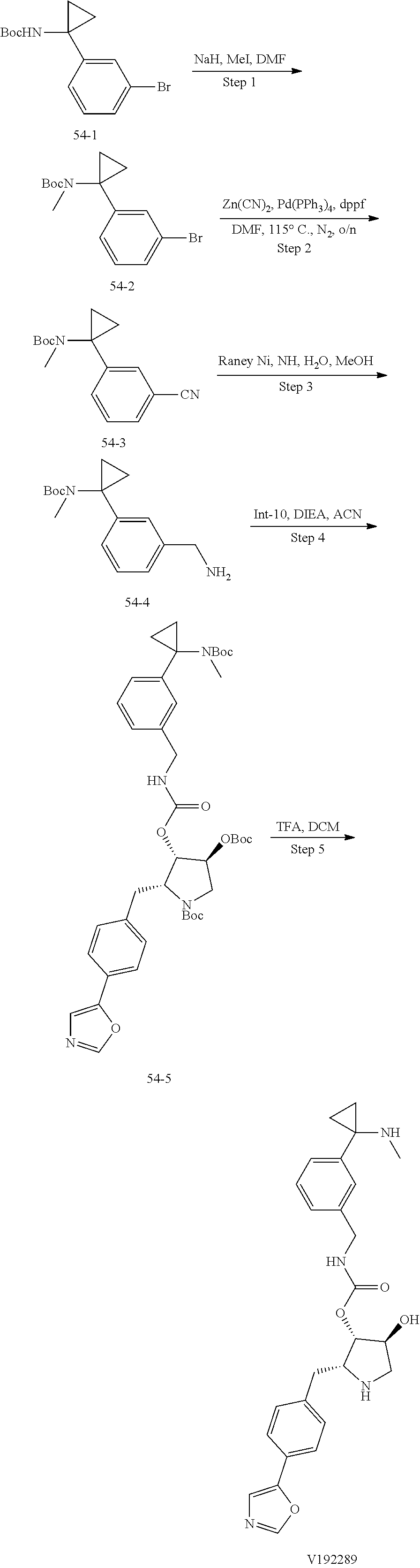

Step 1: To a stirred solution of tert-butyl N-[1-(3-bromophenyl)cyclopropyl]carbamate (500 mg, 1.60 mmol, 1 eq.) in DMF (8 mL) was added NaH (96.0 mg, 4.00 mmol, 2.5 eq.) at 0° C. for 30 min. To the above mixture was added iodomethane (454.6 mg, 3.20 mmol, 2 eq.). The resulting mixture was stirred at room temperature for additional overnight. The reaction was quenched by the addition of Water (20 mL) at 0° C. The resulting mixture was extracted with EtOAc (3×50 mL). The combined organic layers were washed with brine (2×30 mL), dried over anhydrous Na₂SO₄. After filtration, the filtrate was concentrated under reduced pressure. The crude product was purified by reversed phase flash (NH₄HCO₃/H₂O) to afford tert-butyl N-[1-(3-bromophenyl)cyclopropyl]-N-methylcarbamate (525 mg, 99.4% yield, 98% purity) as a yellow oil. MS: m/z: Calc'd for C₁₅H₂₀BrNO₂ [M+H−56]⁺270. Found, 270.

Step 2: To a stirred solution of tert-butyl N-[1-(3-bromophenyl)cyclopropyl]-N-methylcarbamate (380 mg, 1.16 mmol, 1 eq.) in DMF (6 mL) were added zinc dicarbonitrile (205.1 mg, 1.74 mmol, 1.5 eq.) and Pd(PPh₃)₄ (67.3 mg, 0.05 mmol, 0.05 eq.) at room temperature under nitrogen atmosphere. To the above mixture was added Dppf (64.5 mg, 0.11 mmol, 0.1 eq.) and Zinc (7.6 mg, 0.11 mmol, 0.1 eq.). The resulting mixture was stirred at 115° C. under nitrogen atmosphere for additional overnight. The resulting mixture was extracted with EtOAc (3×50 mL). The combined organic layers were washed with brine (2×40 mL), dried over anhydrous Na₂SO₄. After filtration, the filtrate was concentrated under reduced pressure. The crude product was purified by reversed phase flash (NH₄HCO₃/H₂O) to afford tert-butyl N-[1-(3-cyanophenyl)cyclopropyl]-N-methylcarbamate (278 mg, 87.6% yield, 90% purity) as a yellow oil. MS: m/z: Calc'd for C₁₆H₂₀N₂O₂ [M+H−56]⁺217. Found, 217.

Step 3: To a stirred solution of tert-butyl N-[1-(3-cyanophenyl)cyclopropyl]-N-methylcarbamate (140 mg, 0.51 mmol, 1 eq.) in methanol (5 mL) were added raney nickel (90.5 mg, 1.54 mmol, 3.00 eq.) and Ammonium hydroxide (9% in water) (30 mg, 0.85 mmol, 1.67 eq.) at room temperature under hydrogen atmosphere for 2 h. The precipitated solids were collected by filtration and washed with methanol (5 mL) (1×20 mL). The resulting mixture was concentrated under reduced pressure to afford tert-butyl N-{1-[3-(aminomethyl)phenyl]cyclopropyl}-N-methylcarbamate (120 mg) as a white solid. MS: m/z: Calc'd for C₁₆H₂₄N₂O₂ [2M+H]⁺553. Found, 553.

### Step 4 and 5

The title compound was prepared in 35.1% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl N-{1-[3-(aminomethyl)phenyl]cyclopropyl}-N-methylcarbamate (54-3) in STEP 4; Boc Deprotection; General Procedure II in STEP 5. MS: m/z Calc'd for C₂₆H₃₀N₄O₄ [M+H]⁺463. found 463. ¹H NMR (400 MHz, Methanol-d₄) δ 8.28 (s, 1H), 7.75-7.67 (m, 2H), 7.56-7.45 (m, 5H), 7.42 (d, J=8.3 Hz, 2H), 5.03-4.98 (m, 1H), 4.43 (d, J=15.5 Hz, 3H), 4.26-4.24 (m, 1H), 3.69-3.57 (m, 1H), 3.29-3.18 (m, 2H), 3.10 (dd, J=14.0, 8.4 Hz, 1H), 2.60 (s, 3H), 1.47-1.38 (m, 2H), 1.38-1.25 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 30% B in 17 min; Wave Length: 254 nm/220 nm; RT1(min): 16.5.

### Synthetic Procedure of (2R,3S,4S)-4-hydroxy-2-{[4-(1,3-oxazol-5-yl)phenyl]methyl}pyrrolidin-3-yl N-(4,4,4-trifluoro-3-hydroxybutyl)carbamate (V192868)

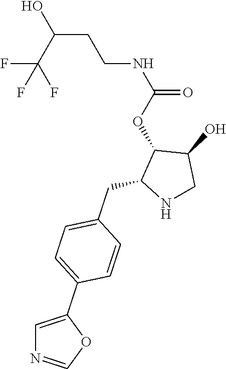 [SMILES: O=C(NCCC(O)C(F)(F)F)O[C@@H]1[C@@H](O)CN[C@@H]1Cc1ccc(-c2cnco2)cc1]

The title compound was prepared in 31.2% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 4-amino-1,1,1-trifluorobutan-2-ol in STEP 1; Boc Deprotection; General Procedure II in STEP 2. MS: m/z Calc'd for C₁₉H₂₂N₃O₃S₅ [M+H]⁺430. found 430. ¹H NMR (400 MHz, Methanol-d₄) δ 8.27 (s, 1H), 7.77 (d, J=8.1 Hz, 2H), 7.54 (d, J=1.1 Hz, 1H), 7.44 (d, J=8.2 Hz, 2H), 4.99 (s, 1H), 4.47 (s, 1H), 4.35-4.28 (m, 1H), 4.01 (dd, J=7.4, 4.0 Hz, 1H), 3.59 (dd, J=12.7, 4.3 Hz, 1H), 3.37 (d, J=8.0 Hz, 2H), 3.29-3.19 (m, 2H), 3.14-3.03 (m, 1H), 1.98-1.89 (m, 1H), 1.77 (s, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 5% B to 35% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-{2-[(trifluoromethyl)sulfanyl]ethyl}carbamate (V192442)

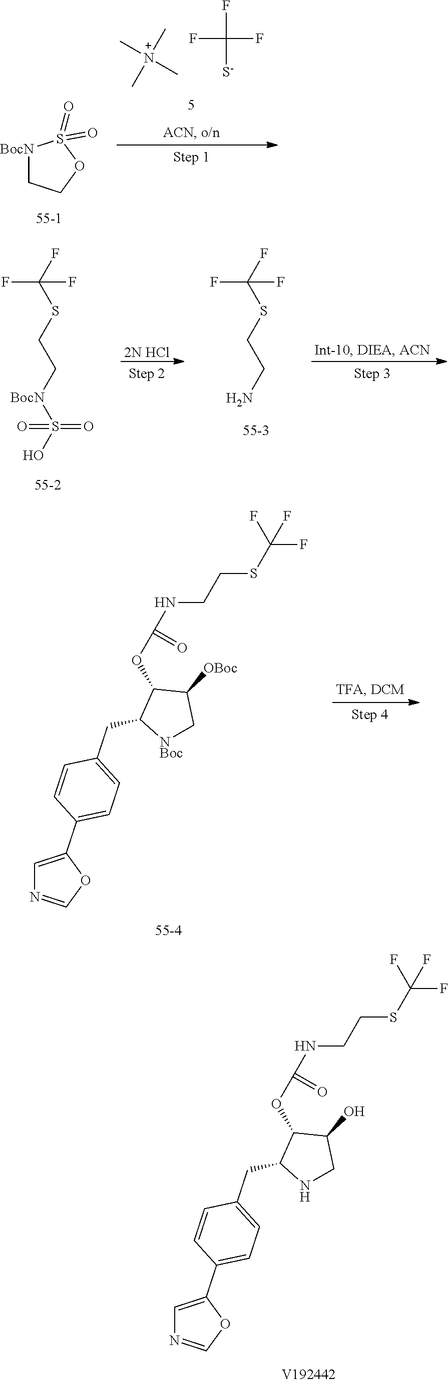

Step 1: To a stirred solution of tert-butyl 2,2-dioxo-1,2lambda6,3-oxathiazolidine-3-carboxylate (600 mg, 2.68 mmol, 1 eq.) and (trifluoromethyl)sulfanide; tetramethylammonium ion (941.8 mg, 5.37 mmol, 2 eq.) in ACN (8 mL) at room temperature. The resulting mixture was stirred at room temperature for overnight. The crude product was purified by reverse phase flash with the following conditions (NH₄HCO₃/H₂O) to afford N-(tert-butoxycarbonyl)-N-sulfo-2-[(trifluoromethyl)sulfanyl]ethanamine (250 mg, 28.5% yield, 95% purity) as a light yellow solid. MS: m/z: Calc'd for C₈H₁₄F₃NO₅S₂[M−H]-324. Found, 324.

Step 2: To a stirred solution of N-(tert-butoxycarbonyl)-N-sulfo-2-[(trifluoromethyl)sulfanyl]ethanamine (245 mg, 0.75 mmol, 1 eq.) in HCl (1 mL) H₂O (5 mL, 277.54 mmol) at 0° C. The resulting mixture was stirred at room temperature for overnight. The resulting mixture was concentrated under reduced pressure to afford 2-[(trifluoromethyl)sulfanyl]ethanamine (200 mg) as an off-white solid. MS: m/z: Calc'd for C₃H₆F₃NS [M+H]⁺146. Found, 146.

Step 3 and 4: The title compound was prepared in 11.2% overall yield as a yellow solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 2-[(trifluoromethyl)sulfanyl]ethanamine (55-3) in STEP 3; Boc Deprotection; General Procedure II in STEP 4. MS: m/z Calc'd for C₁₈H₂₀F₃N₃O₄S[M+H]⁺432. Found, 432. ¹H NMR (400 MHz, Methanol-d₄) δ 8.27 (s, 1H), 7.76 (d, J=8.1 Hz, 2H), 7.54 (s, 1H), 7.44 (d, J=8.2 Hz, 2H), 5.00 (s, 1H), 4.41 (d, J=4.3 Hz, 1H), 4.27 (s, 1H), 3.57 (dd, J=12.6, 4.2 Hz, 1H), 3.47 (d, J=3.7 Hz, 2H), 3.25 (d, J=12.3 Hz, 2H), 3.25-3.14 (m, 2H), 3.08 (dd, J=14.1, 8.8 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 15% B to 45% B in 10 min; Wave Length: 254 nm/220 nm; RT1(min): 7.5.

### Synthetic Procedure of (2R,3S,4S)-4-hydroxy-2-(pyrazol-1-ylmethyl)pyrrolidin-3-yl N-benzylcarbamate (V188209)

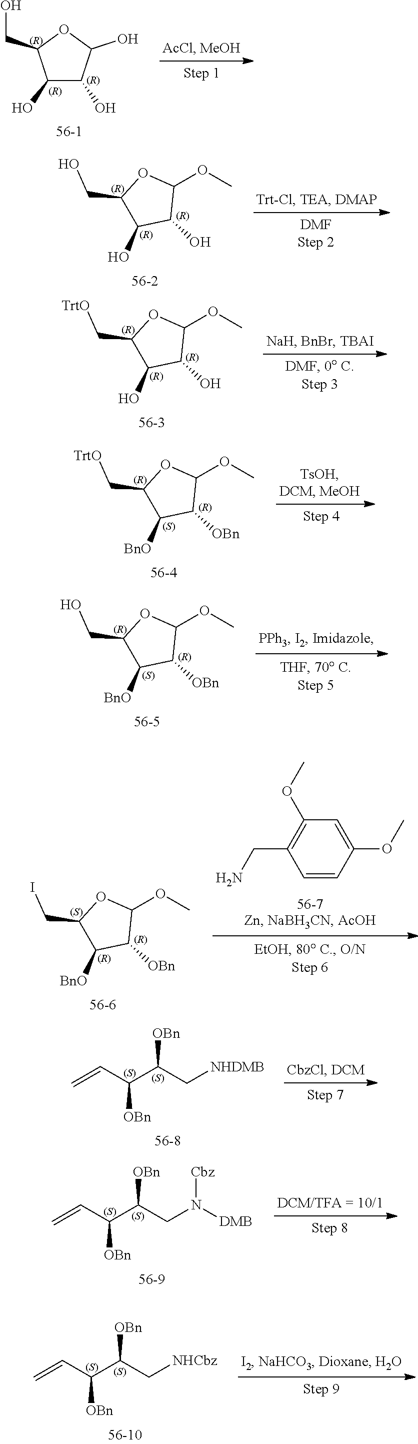

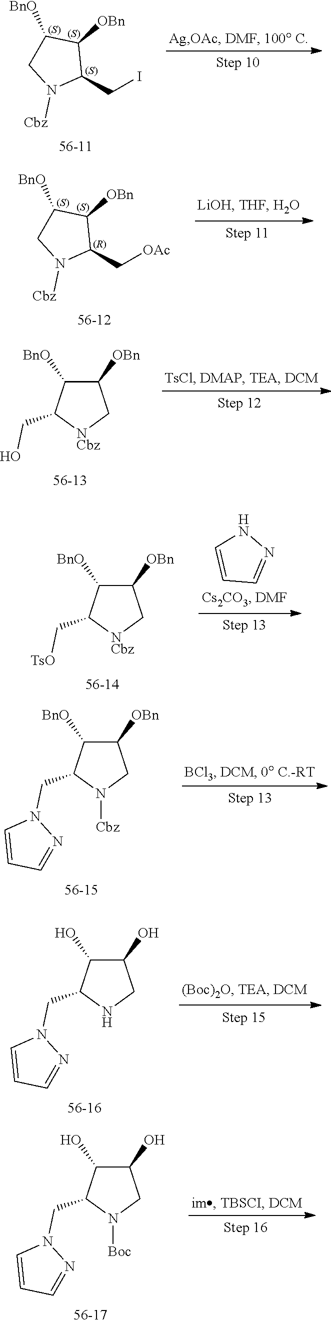

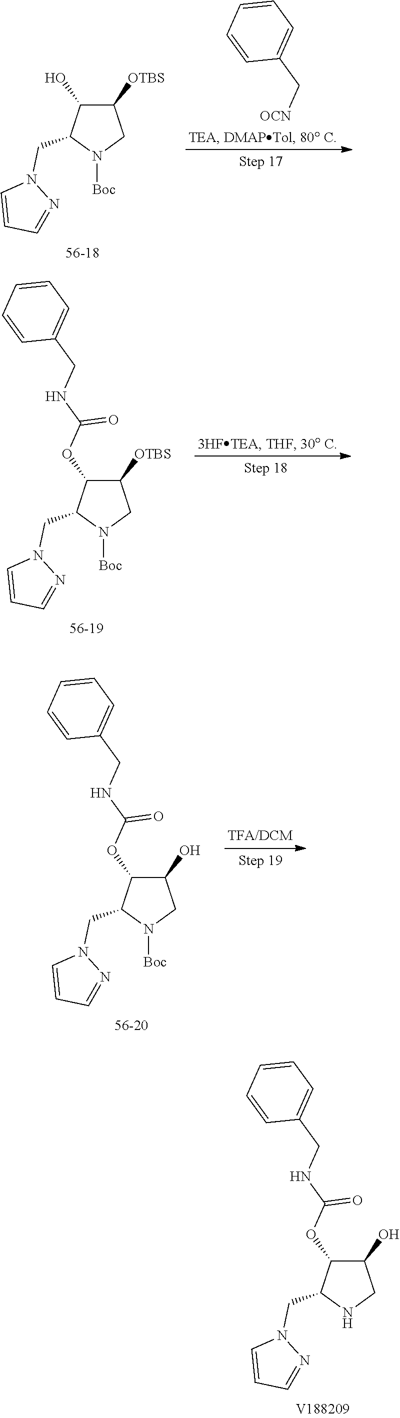

Step 1: To a stirred solution of (3R,4R,5R)-5-(hydroxymethyl)oxolane-2,3,4-triol (30 g, 199.82 mmol, 1 eq.) in methanol (600 mL) were added acetyl chloride (6 mL, 76.43 mmol) at 0° C. The resulting mixture was stirred at room temperature for overnight. The mixture was neutralized to pH 7 with saturated NaHCO₃ (aq.). The resulting mixture was concentrated under reduced pressure. The residue was purified by silica gel column chromatography, eluted with EA/MeOH (9:1) to afford (2R,3R,4R)-2-(hydroxymethyl)-5-methoxyoxolane-3,4-diol (30 g, 91.4% yield, 95% purity) as a light yellow oil. MS: m/z: Calc'd for C₆H₁₂O₅ [M+H]⁺165.

Step 2: To a stirred solution of (2R,3R,4R)-2-(hydroxymethyl)-5-methoxyoxolane-3,4-diol (30 g, 182.75 mmol, 1 eq.) and (chlorodiphenylmethyl)benzene (127.3 g, 456.88 mmol, 2.5 eq.) in DMF (1000 mL) were added Et₃N (46.2 g, 456.88 mmol, 2.5 eq.) and DMAP (2.2 g, 18.27 mmol, 0.1 eq.) at 0° C. The resulting mixture was stirred at 50° C. for overnight. The resulting mixture was extracted with EtOAc (2×1000 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EA (3:1) to afford (3R,4R,5R)-2-methoxy-5-[(triphenylmethoxy)methyl]oxolane-3,4-diol (40 g, 53.8% yield, 95% purity) as a light yellow oil. MS: m/z: Calc'd for C₂₅H₂₆O₅[M−H]⁻405. Found, 405.

Step 3: To a stirred solution of (3R,4R,5R)-2-methoxy-5-[(triphenylmethoxy)methyl]oxolane-3,4-diol (40 g, 98.40 mmol, 1 eq.) and (bromomethyl)benzene (42.0 g, 246.01 mmol, 2.5 eq.) in DMF (600 mL) were added tetrabutylazanium iodide (1.8 g, 4.92 mmol, 0.05 eq.) and NaH (10.3 g, 432.98 mmol, 4.4 eq.) at 0° C. The resulting mixture was stirred at room temperature for overnight. The reaction was quenched with Water at 0° C. The resulting mixture was extracted with EtOAc (2×500 mL). The combined organic layers were washed with brine (1×300 mL), dried over anhydrous Na₂SO₄. After filtration, the filtrate was concentrated under reduced pressure. The residue was purified by silica gel column chromatography, eluted with PE/EA (3:1) to afford (3R,4S,5R)-3,4-bis(benzyloxy)-2-methoxy-5-[(triphenylmethoxy)methyl]oxolane (50 g, 86.6% yield, 95% purity) as a light yellow oil. MS: m/z: Calc'd for C₃₉H₃₈O₅ [M+H+17]⁺604. Found, 604.

Step 4: To a stirred solution of (3R,4S,5R)-3,4-bis(benzyloxy)-2-methoxy-5-[(triphenylmethoxy)methyl]oxolane (50 g, 85.21 mmol, 1 eq.) in MEOH (500 mL) and DCM (500 mL) were added TSOH (73.3 g, 426.09 mmol, 5 eq.) at 0° C. The resulting mixture was stirred at room temperature for overnight. The mixture was neutralized to pH 7 with saturated NaHCO₃ (aq.). The residue was purified by silica gel column chromatography, eluted with PE/EA (3:2) to afford [(2R,3S,4R)-3,4-bis(benzyloxy)-5-methoxyoxolan-2-yl]methanol (20 g, 68.1% yield, 95% purity) as a light yellow oil. MS: m/z: Calc'd for C₂₀H₂₄O₅[M+H+17]⁺362. Found, 362.

Step 5: To a stirred solution of [(2R,3S,4R)-3,4-bis(benzyloxy)-5-methoxyoxolan-2-yl]methanol (20 g, 58.07 mmol, 1 eq.) and Imidazole (7.9 g, 116.14 mmol, 2 eq.) in THF (500 mL) were added PPh₃ (22.8 g, 87.10 mmol, 1.5 eq.) and iodine (22.1 g, 87.10 mmol, 1.5 eq.) at 0° C. The resulting mixture was stirred at 70° C. under nitrogen atmosphere for 2 h. The resulting mixture was extracted with EtOAc (2×500 mL). The combined organic layers were washed with brine (2×400 mL), dried over anhydrous Na₂SO₄. After filtration, the filtrate was concentrated under reduced pressure. The residue was purified by silica gel column chromatography, eluted with PE/EA (4:1) to afford (2S,3R,4R)-3,4-bis(benzyloxy)-2-(iodomethyl)-5-methoxyoxolane (20 g, 75.8% yield, 95% purity) as a light yellow oil. MS: m/z: Calc'd for C₂₀H₂₃IO₄ [M+H+22]⁺477. found 477.

Step 6: To a stirred solution of (2S,3R,4R)-3,4-bis(benzyloxy)-2-(iodomethyl)-5-methoxyoxolane (2000 mg, 4.41 mmol, 1 eq.) and 1-(2,4-dimethoxyphenyl)methanamine (1472.2 mg, 8.81 mmol, 2 eq.) in EtOH (25 mL) were added Zn (1439.1 mg, 22.01 mmol, 5 eq.) and AcOH (528.7 mg, 8.80 mmol, 2 eq.). NaBH₃CN (553.3 mg, 8.80 mmol, 2 eq.) was added the mixture slowly at 0° C. and then stirred at 80° C. for overnight. LCMS showed the reaction was completed. The mixture was filtered and washed with EA. Filtrate was concentrated and purified by column chromatography, eluted with PE/EA (2:1) to afford [(2S,3S)-2,3-bis(benzyloxy)pent-4-en-1-yl][(2,4-dimethoxyphenyl)methyl]amine (1800 mg, 91% yield, 95% purity) as a yellow oil. MS: m/z: Calc'd for C₂₈H₃₃NO₄ [M+H]⁺448. Found 448.

Step 7: To a stirred solution of [(2S,3S)-2,3-bis(benzyloxy)pent-4-en-1-yl][(2,4-dimethoxyphenyl)methyl]amine (1800 mg, 4.02 mmol, 1 eq.) and NaHCO₃ (1351.4 mg, 16.09 mmol, 4 eq.) in 1,4-dioxane (20 mL) and H₂O (10 mL) was added CbzCl (1372.1 mg, 8.04 mmol, 2 eq.) at 0° C. Then the mixture was stirred at room temperature for 3h. LCMS showed the reaction was completed. The mixture was diluted with water and extracted with EA, the combined organic layers were washed with brine and dried over anhydrous sodium sulfate. After filtration, the filtrate was concentrated under reduced pressure. The resulting residue was purified by column chromatography, eluted with PE/EA (4:1) to afford benzyl N-[(2S,3S)-2,3-bis(benzyloxy)pent-4-en-1-yl]-N-[(2,4-dimethoxyphenyl)methyl]carbamate (1600 mg, 68% yield, 95% purity) as a yellow oil. MS: m/z: Calc'd for C₃₆H₃₉NO₆ [M+H+22]⁺604. Found 604.

Step 8: To a stirred solution of benzyl N-[(2S,3S)-2,3-bis(benzyloxy)pent-4-en-1-yl]-N-[(2,4-dimethoxyphenyl)methyl]carbamate (1600 mg, 2.75 mmol, 1 eq.) in DCM (20 mL) was added TFA (2 mL) and the mixture was stirred at room temperature for 1 h. LCMS showed the reaction was completed. The mixture was quenched with saturated aqueous NaHCO₃ and extracted with EA. The combined organic layers were washed with brine and dried over anhydrous sodium sulfate. After filtration, the filtrate was concentrated under reduced pressure. The resulting residue was purified by column chromatography, eluted with PE/EA (2:1) to afford benzyl N-[(2S,3S)-2,3-bis(benzyloxy)pent-4-en-1-yl]carbamate (1150 mg, 98% yield, 95% purity) as a yellow solid. MS: m/z: Calc'd for C₂₇H₂₉NO₄ [M+H]⁺432. Found, 432.

Step 9: To a stirred solution of benzyl N-[(2S,3S)-2,3-bis(benzyloxy)pent-4-en-1-yl]carbamate (1150 mg, 2.66 mmol, 1 eq.) in Dioxane (15 mL) and water (5 mL) were added 1₂ (2705.5 mg, 10.66 mmol, 4 eq.) and NaHCO₃ (895.5 mg, 10.66 mmol, 4 eq.) and the mixture was stirred at room temperature for overnight. LCMS showed the reaction was completed. The mixture was diluted with water and extracted with EA, the combined organic layers were washed with brine and dried over anhydrous sodium sulfate. After filtration, the filtrate was concentrated under reduced pressure. The resulting residue was purified by column chromatography eluted with PE/EA (2:1) to afford benzyl (2S,3S,4S)-3,4-bis(benzyloxy)-2-(iodomethyl)pyrrolidine-1-carboxylate (1150 mg, 76% yield, 95% purity) as a yellow oil. MS: m/z: Calc'd for C₂₇H₂₈INO₄ [M+H]⁺558. Found 558.

Step 10: A mixture of benzyl (2R,3S,4S)-2-[(acetyloxy)methyl]-3,4-bis(benzyloxy)pyrrolidine-1-carboxylate (1 g, 2.03 mmol, 1 eq) and Silver acetate (545.5 mg, 3.26 mmol, 1.6 eq) in DMF (10 mL) was stirred for overnight at 100° C. under nitrogen atmosphere. Desired product could be detected by LCMS. The resulting mixture was diluted with water (10 mL). The resulting mixture was extracted with EtOAc (3×30 mL). The combined organic layers were dried over anhydrous Na₂SO₄. After filtration, the filtrate was concentrated under reduced pressure. The residue was purified by reversed-phase flash chromatography to afford benzyl (2S,3S,4S)-3,4-bis(benzyloxy)-2-(iodomethyl)pyrrolidine-1-carboxylate (430 mg, 37.7%) as a light yellow oil. MS: m/z: Calc'd for C₂₉H₃₁NO₆ [M+H]⁺490. found 490.

Step 11: A mixture of benzyl (2R,3S,4S)-3,4-bis(benzyloxy)-2-(hydroxymethyl)pyrrolidine-1-carboxylate (420 mg, 0.93 mmol, 1 eq) and LiOH (112.38 mg, 4.69 mmol, 5 eq) in THF (6 mL) and H₂O (1 mL) was stirred for overnight at room temperature under nitrogen atmosphere. Desired product could be detected by LCMS. The resulting mixture was diluted with water (10 mL). The mixture was neutralized to pH 6 with 1M HCl (aq.). The resulting mixture was extracted with EtOAc (3×30 mL). The combined organic layers were dried over anhydrous Na₂SO₄. After filtration, the filtrate was concentrated under reduced pressure. The residue was purified by reversed-phase flash chromatography to afford benzyl (2R,3S,4S)-2-[(acetyloxy)methyl]-3,4-bis(benzyloxy)pyrrolidine-1-carboxylate (400 mg, 87.1%) as a light yellow oil. MS: m/z: Calc'd for C₂₇H₂₉NO₅ [M+H]⁺448. found 448.

Step 12: A mixture of benzyl (2R,3S,4S)-3,4-bis(benzyloxy)-2-(hydroxymethyl)pyrrolidine-1-carboxylate (185 mg, 0.41 mmol, 1 eq), P-toluenesulfonyl chloride (94.5 mg, 0.49 mmol, 1.2 eq), TEA (125.4 mg, 1.23 mmol, 3 eq) and DMAP (50.5 mg, 0.41 mmol, 1 eq) in DCM (4 mL) was stirred for overnight at room temperature under nitrogen atmosphere. Desired product could be detected by LCMS. The resulting mixture was concentrated under vacuum. The residue was purified by reverse phase flash with the following conditions (5.0 mmol/L NH₄HCO₃/ACN) to afford benzyl (2R,3S,4S)-3,4-bis(benzyloxy)-2-{[(4-methylbenzenesulfonyl)oxy]methyl}pyrrolidine-1-carboxylate (165 mg, 66.3%) as a light yellow oil. MS: m/z: Calc'd for C₃₄H₃₅NO₇S [M+H]⁺602. found 602.

Step 13: To a stirred mixture of benzyl (2R,3S,4S)-3,4-bis(benzyloxy)-2-{[(4-methylbenzenesulfonyl)oxy]methyl}pyrrolidine-1-carboxylate (240 mg, 0.39 mmol, 1 eq) and pyrazole (54.3 mg, 0.79 mmol, 2 eq) in DMF (3 mL) was added Cs₂CO₃ (0.80 mL, 0.79 mmol, 3 eq) at room temperature under nitrogen atmosphere. The resulting mixture was stirred at 80° C. for overnight. Desired product could be detected by LCMS. The resulting mixture was purified by reverse phase flash with the following conditions (5.0 mmol/LNH₄HCO₃/ACN) to obtain benzyl (2R,3S,4S)-3,4-bis(benzyloxy)-2-(pyrazol-1-ylmethyl)pyrrolidine-1-carboxylate (175 mg, 88.1%) as a light yellow solid. MS: m/z: Calc'd for C₃₀H₃₁N₃O₄ [M+H]⁺498. found 498.

Step 14: To a stirred solution of benzyl (2R,3S,4S)-3,4-bis(benzyloxy)-2-(pyrazol-1-ylmethyl)pyrrolidine-1-carboxylate (200 mg, 0.40 mmol, 1 eq) in DCM (2 mL) was added boron trichloride (5 mL) dropwise at room temperature under nitrogen atmosphere. The resulting mixture was stirred for 2 h at room temperature. Desired product could be detected by LCMS. The resulting mixture was diluted with MeOH (3 mL) and directly concentrated under vacuum to obtain the crude (2R,3S,4S)-2-(pyrazol-1-ylmethyl)pyrrolidine-3,4-diol (80 mg, 108.6%) as a light yellow oil. MS: m/z: Calc'd for C₈H₁₃N₃O₂ [M+H]⁺ 184. found 184.

Step 15: A mixture of tert-butyl (2R,3S,4S)-3,4-dihydroxy-2-(pyrazol-1-ylmethyl)pyrrolidine-1-carboxylate (75 mg, 0.26 mmol, 1 eq), di-tert-butyl dicarbonate (107.2 mg, 0.49 mmol, 1.2 eq) and TEA (124.2 mg, 1.22 mmol, 3 eq) in DCM (5 mL) was stirred for overnight at room temperature under nitrogen atmosphere. Desired product could be detected by LCMS. The resulting mixture was concentrated under vacuum. The residue was purified by reverse phase flash with the following conditions (5.0 mmol/L NH₄HCO₃/ACN) to afford tert-butyl (2R,3S,4S)-3,4-dihydroxy-2-(pyrazol-1-ylmethyl)pyrrolidine-1-carboxylate (75 mg, 64.6%) as a light yellow oil. MS: m/z: Calc'd for C₁₃H₂₁N₃O₄ [M+H]⁺ 284. found 284.

Step 16: A mixture of tert-butyl (2R,3S,4S)-3,4-dihydroxy-2-(pyrazol-1-ylmethyl)pyrrolidine-1-carboxylate (75 mg, 0.26 mmol, 1 eq), t-butyldimethylchlorosilane (79.7 mg, 0.53 mmol, 2 eq) and imidazole (36. mg, 0.53 mmol, 2 eq) in DCM (3 mL) was stirred for overnight at room temperature under nitrogen atmosphere. Desired product could be detected by LCMS. The resulting mixture was concentrated under vacuum. The residue was purified by Prep-TLC (PE/EA 1:1) to afford tert-butyl (2R,3S,4S)-3-[(benzylcarbamoyl)oxy]-4-[(tert-butyldimethylsilyl)oxy]-2-(pyrazol-1-ylmethyl)pyrrolidine-1-carboxylate (50 mg, 47.5%) as a light yellow solid. MS: m/z: Calc'd for C₁₉H₃₅N₃O₄Si [M+H]⁺ 398. found 398.

Step 17: A mixture of tert-butyl (2R,3S,4S)-4-[(tert-butoxycarbonyl)oxy]-3-hydroxy-2-{[4-(1,3-oxazol-2-yl)phenyl]methyl}pyrrolidine-1-carboxylate (75 mg, 0.16 mmol, 1 eq), 1-fluoro-3-(isocyanatomethyl)benzene (34.2 mg, 0.22 mmol, 2 eq), TEA (49.4 mg, 0.48 mmol, 3 eq) and DMAP (13.8 mg, 0.11 mmol, 1 eq) in Toluene (3 mL) was stirred for overnight at 80° C. under nitrogen atmosphere. Desired product could be detected by LCMS. The resulting mixture was concentrated under vacuum. The residue was purified by Prep-TLC (PE/EA 2:1) to afford tert-butyl (2R,3S,4S)-3-[(benzylcarbamoyl)oxy]-4-[(tert-butyldimethylsilyl)oxy]-2-(pyrazol-1-ylmethyl)pyrrolidine-1-carboxylate (60 mg, 99.8%) as a light yellow oil. MS: m/z: Calc'd for C₂₇H₄₂N₄O₅Si [M+H]⁺ 531. found 531.

Step 18: To a stirred solution of tert-butyl (2R,3S,4S)-3-[(benzylcarbamoyl)oxy]-4-[(tert-butyldimethylsilyl)oxy]-2-(pyrazol-1-ylmethyl)pyrrolidine-1-carboxylate (55 mg, 0.104 mmol) in THF (2 mL) was added TEA·3HF (2 mL) dropwise at room temperature under nitrogen atmosphere. The resulting mixture was stirred for 2 h at room temperature. Desired product could be detected by LCMS. The resulting mixture was diluted with DCM (10 mL) and directly concentrated under vacuum to obtain the crude (2R,3S,4S)-4-hydroxy-2-(pyrazol-1-ylmethyl)pyrrolidin-3-yl N-benzylcarbamate; trifluoroacetic acid (24.3 mg, 46.6%) as a light yellow oil. MS: m/z: Calc'd for C₂₁H₂₈N₄O₅ [M+H]⁺ 417. found 417.

Step 19: The title compound was prepared in 46.6% yield as a white solid according to Boc Deprotection; General Procedure II in STEP 19. MS: m/z Calc'd for C₁₆H₂₀N₄O₃ [M+H]⁺ 317, found 317. ¹H NMR (400 MHz, Methanol-d₄) δ 7.65 (d, J=2.4 Hz, 1H), 7.59 (d, J=1.9 Hz, 1H), 7.42-7.19 (m, 5H), 6.33 (q, J=1.9 Hz, 1H), 5.18 (d, J=3.5 Hz, 1H), 4.74-4.66 (m, 1H), 4.57-4.45 (m, 1H), 4.45-4.40 (m, 2H), 4.33 (d, J=2.3 Hz, 2H), 3.66-3.58 (m, 1H), 3.28 (s, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 4% B to 34% B in 10 min; Wave Length: 254 nm/220 nm; RT1(min): 8.5.

### Synthetic Procedure of (2R,3S,4S)-4-hydroxy-2-(pyridin-3-ylmethyl)pyrrolidin-3-yl N-[(3-fluorophenyl)methyl]carbamate (V190519)

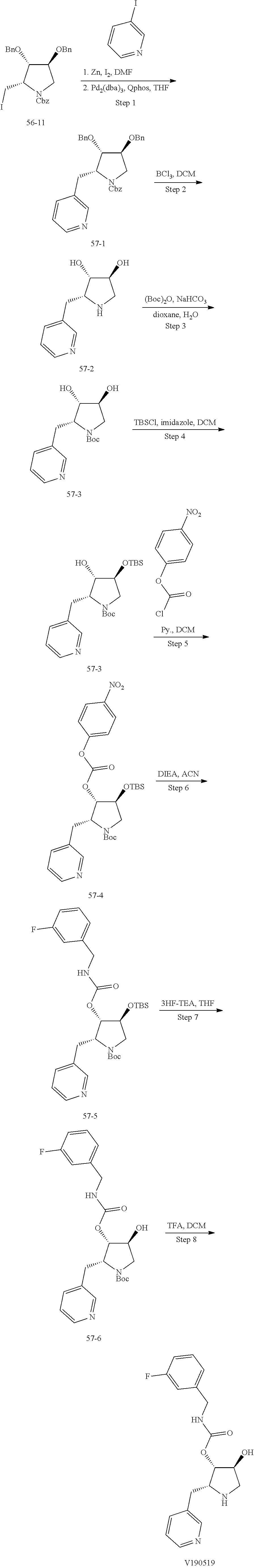

Step 1: A solution of benzyl (2S,3S,4S)-3,4-bis(benzyloxy)-2-(iodomethyl)pyrrolidine-1-carboxylate (1 g, 1.79 mmol, 1.0 eq.) in DMF (10 mL) was treated with Zn (1.17 g, 17.94 mmol, 10 eq.) and 1₂ (0.91 g, 3.59 mmol, 2.0 eq.) for 1 h at 25° C. under nitrogen atmosphere. Then a solution of 3-iodopyridine (0.74 g, 3.59 mmol, 2.0 eq.) in THF (10 mL) was treated with Pd₂(dba)₃ (0.16 g, 0.18 mmol, 0.1 eq.) and 1,2,3,4,5-pentaphenyl-1′-(di-tert-butylphosphino) (0.25 g, 0.36 mmol, 0.2 eq.) under N₂. To the above mixture was added the first crude product dropwise 2 min at 25° C. The resulting mixture was stirred for additional 12 h at 25° C. under N₂. The resulting mixture was extracted with EtOAc (3×10 mL). The combined organic layers were washed with water (3×5 mL), dried over anhydrous Na₂SO₄. After filtration, the filtrate was concentrated under reduced pressure. The residue was purified by silica gel column chromatography, eluted with PE/EA (1:1) to afford benzyl (2R,3S,4S)-3,4-bis(benzyloxy)-2-(pyridin-3-ylmethyl)pyrrolidine-1-carboxylate as a light yellow oil. MS: m/z: Calc'd for C₃₂H₃₂N₂O₄ [M+H]⁺509. Found, 509.

Step 2: To a stirred solution of benzyl (2R,3S,4S)-3,4-bis(benzyloxy)-2-(pyridin-3-ylmethyl)pyrrolidine-1-carboxylate (400 mg, 0.79 mmol, 1.0 eq.) in DCM (20 mL) was added boron trichloride (11.8 mL, 11.79 mmol, 15 equiv) in portions at 0° C. under N₂. The resulting mixture was stirred over night at 25° C. under a nitrogen atmosphere. Desired product could be detected by LCMS. The reaction was quenched with MeOH at 0° C. The resulting mixture was concentrated under reduced pressure. The resulting mixture was used in the next step directly without further purification. MS: m/z: Calc'd for C₁₀H₁₄N₂O₂ [M+H]⁺195. Found, 195.

Step 3: To a stirred solution of (2R,3S,4S)-2-(pyridin-3-ylmethyl)pyrrolidine-3,4-diol (255 mg, 1.31 mmol, 1.0 eq.) in dioxane (6 mL) and H₂O (2 mL) was added (Boc)₂O (573.0 mg, 2.63 mmol, 2.0 eq.) and NaHCO₃ (441.2 mg, 5.25 mmol, 4.0 eq.) in portions at 0° C. The resulting mixture was stirred over night at 25° C. Desired product could be detected by LCMS. The residue was purified by reversed-phase flash chromatography to afford tert-butyl (2R,3S,4S)-3,4-dihydroxy-2-(pyridin-3-ylmethyl)pyrrolidine-1-carboxylate (270 mg, 69.9%) as a light yellow oil. MS: m/z: Calc'd for C₁₅H₂₂N₂O₄ [M+H]⁺295. Found, 295.

Step 4: To a stirred solution of tert-butyl (2R,3S,4S)-3,4-dihydroxy-2-(pyridin-3-ylmethyl)pyrrolidine-1-carboxylate (260 mg, 0.88 mmol, 1.0 eq.) in DCM (6 mL) was added TBSCl (266.3 mg, 1.77 mmol, 2.0 eq.) and Imidazole (180.4 mg, 2.65 mmol, 3.0 eq.) in portions at 0° C. The resulting mixture was stirred over night at 25° C. under a nitrogen atmosphere. The residue was purified by Prep-TLC (PE/EA 1:1) to afford tert-butyl (2R,3S,4S)-4-[(tert-butyldimethylsilyl)oxy]-3-hydroxy-2-(pyridin-3-ylmethyl)pyrrolidine-1-carboxylate (300 mg, 83.1%) as a light yellow oil. MS: m/z: Calc'd for C₂₁H₃₆N₂O₄Si [M+H]⁺409. Found, 409.

Step 5: To a stirred solution of tert-butyl (2R,3S,4S)-4-[(tert-butyldimethylsilyl)oxy]-3-hydroxy-2-(pyridin-3-ylmethyl)pyrrolidine-1-carboxylate (105 mg, 0.25 mmol, 1 eq.) and 4-nitrophenyl carbonochloridate (103.5 mg, 0.51 mmol, 2 eq.) in DCM (3 mL) were added Pyridine (60.9 mg, 0.77 mmol, 3 eq.) at 0° C. The resulting mixture was stirred at room temperature for overnight. The resulting mixture was concentrated under reduced pressure. The residue was purified by Prep-TLC (PE/EA 1:1) to afford tert-butyl (2R,3S,4S)-4-[(tert-butyldimethylsilyl)oxy]-3-[(4-nitrophenoxycarbonyl)oxy]-2-(pyridin-3-ylmethyl)pyrrolidine-1-carboxylate (165 mg, 111.9%) as a yellow oil. MS: m/z: Calc'd for C₂₈H₃₉N₃O₈Si[M+H]⁺574. Found, 574.

Step 6: The compound was prepared in 51.2% yield as a yellow oil according to Substitution reaction for synthesis of Carbamate; General Procedure I using tert-butyl (2R,3S,4S)-4-[(tert-butyldimethylsilyl)oxy]-3-[(4-nitrophenoxycarbonyl)oxy]-2-(pyridin-3-ylmethyl)pyrrolidine-1-carboxylate (57-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine. MS: m/z: Calc'd for C₂₉H₄₂FN₃O₅Si[M+H]⁺560. Found, 560.

Step 7: To a stirred solution of tert-butyl (2R,3S,4S)-4-[(tert-butyldimethylsilyl)oxy]-3-({[(3-fluorophenyl)methyl]carbamoyl}oxy)-2-(pyridin-3-ylmethyl)pyrrolidine-1-carboxylate (75 mg, 0.13 mmol, 1 eq.) in THF (3 mL) were added Et₃N·3HF (1 mL, 7.36 mmol) at 0° C. The resulting mixture was stirred at room temperature for overnight. The crude product was purified by reverse phase flash with the following conditions (TFA/H₂O) to afford tert-butyl (2R,3S,4S)-3-({[(3-fluorophenyl)methyl]carbamoyl}oxy)-4-hydroxy-2-(pyridin-3-ylmethyl)pyrrolidine-1-carboxylate (63 mg, 105.5%) as a light yellow oil. MS: m/z: Calc'd for C₂₃H₂₈FN₃O₅[M+H]⁺446. Found, 446.

Step 8: The title compound was prepared in 31.3% yield as an off-white solid according to Boc Deprotection; General Procedure II in STEP 8. MS: m/z Calc'd for C₁₈H₂₀FN₃O₃[M+H]⁺346. Found, 346. ¹H NMR (400 MHz, Methanol-d₄) δ 8.75 (s, 2H), 8.31-8.20 (m, 1H), 7.84-7.74 (m, 1H), 7.37 (dd, J=7.9, 5.8 Hz, 1H), 7.15 (d, J=7.7 Hz, 1H), 7.12-7.08 (m, 1H), 7.02-6.97 (m, 1H), 5.07 (d, J=3.6 Hz, 1H), 4.44-4.35 (m, 4H), 3.65 (dd, J=12.6, 4.0 Hz, 1H), 3.37 (d, J=14.7 Hz, 1H), 3.28 (d, J=12.2 Hz, 2H).

Prep-HPLC purification conditions: Column: Xselect CSH Prep C18 OBD Column, 30*150 mm, 5 μm; Mobile Phase A: Water (0.05% TFA), Mobile Phase B: ACN; Flow rate: 60 mL/min; Gradient: 2% B to 18% B in 7 min; Wave Length: 254 nm/220 nm; RT1(min): 6.05.

### Synthetic Procedure of (2R,3S,4S)-2-[(4-chlorophenyl)methyl]-4-hydroxypyrrolidin-3-yl N-[(3S)-pyrrolidin-3-ylmethyl]carbamate (V188188)

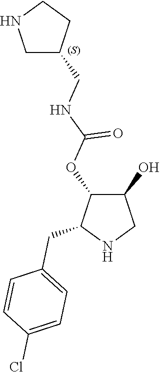

The title compound was prepared in 26.1% overall yield as a yellow solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (3R)-3-(aminomethyl)pyrrolidine-1-carboxylate in STEP 1; Boc Deprotection; General Procedure II in STEP 2. MS: m/z Calc'd for C₁₇H₂₄ClN₃O₃[M+H]⁺354; found 354. ¹H NMR (400 MHz, Methanol-d₄) δ 7.42-7.30 (m, 4H), 5.00-4.94 (m, 1H), 4.39 (d, J=4.2 Hz, 1H), 4.21-4.11 (m, 1H), 3.60-3.52 (m, 1H), 3.48-3.43 (m, 1H), 3.43-3.36 (m, 1H), 3.27-3.25 (m, 3H), 3.23-3.13 (m, 2H), 3.11-2.98 (m, 2H), 2.61-2.51 (m, 1H), 2.19-2.08 (m, 1H), 1.78-1.68 (m, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 3% B to 33% B in 15 min; Wave Length: 254 nm/220 nm; RT1(min): 16.47.

### Synthetic Procedure of (2R,3S,4S)-2-[(4-chlorophenyl)methyl]-4-hydroxypyrrolidin-3-yl N-benzylcarbamate (V188203)

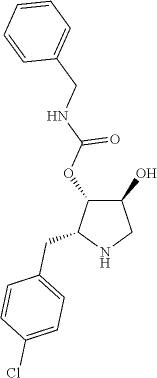 [SMILES: O=C(NCc1ccccc1)O[C@@H]1[C@@H](O)CN[C@@H]1Cc1ccc(Cl)cc1]

The title compound was prepared in 45.8% overall yield as a white solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), benzylamine in STEP 1; Boc Deprotection; General Procedure II in STEP 2. MS: m/z Calc'd for C₁₉H₂₁ClN₂O₃[M+H]⁺361. Found, 361. ¹H NMR (400 MHz, Methanol-d₄) δ 7.41-7.24 (m, 9H), 4.95 (d, J=3.6 Hz, 1H), 4.43-4.30 (m, 3H), 4.21 (dd, J=7.9, 3.5 Hz, 1H), 3.60 (dd, J=12.6, 4.3 Hz, 1H), 3.24-3.16 (m, 2H), 3.05 (dd, J=14.0, 8.2 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 16% B to 46% B in 10 min; Wave Length: 254 nm/220 nm; RT1(min): 9.13.

### Synthetic Procedure of (2R,3S,4S)-2-[(4-chlorophenyl)methyl]-4-hydroxypyrrolidin-3-yl N-[(3-fluorophenyl)methyl]carbamate (V188270)

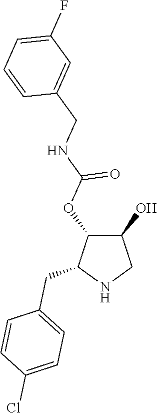 [SMILES: O=C(NCc1cccc(F)c1)O[C@@H]1[C@@H](O)CN[C@@H]1Cc1ccc(Cl)cc1]

The title compound was prepared in 54.6% overall yield as a white solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z Calc'd for C₁₉H₂₀ClFN₂O₃[M+H]⁺379. Found, 379. ¹H NMR (400 MHz, Methanol-d₄) δ 7.44-7.31 (m, 3H), 7.29 (d, J=8.5 Hz, 2H), 7.16-6.99 (m, 3H), 4.96 (d, J=3.6 Hz, 1H), 4.44-4.31 (m, 3H), 4.22-4.20 (m, 1H), 3.59 (dd, J=12.7, 4.3 Hz, 1H), 3.25-3.17 (m, 2H), 3.05 (dd, J=14.0, 8.3 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 17% B to 47% B in 10 min; Wave Length: 254 nm/220 nm; RT1(min): 9.15.

### Synthetic Procedure of (2R,3S,4S)-2-[(4-chlorophenyl)methyl]-4-hydroxypyrrolidin-3-yl N-[(4-fluorophenyl)methyl]carbamate (V188271)

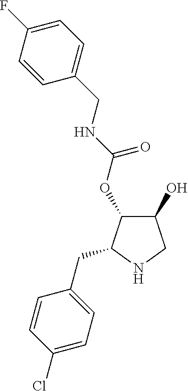 [SMILES: O=C(NCc1ccc(F)cc1)O[C@@H]1[C@@H](O)CN[C@@H]1Cc1ccc(Cl)cc1]

The title compound was prepared in 25.1% overall yield as a white solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 4-fluorobenzylamine in STEP 1; Boc Deprotection; General Procedure II in STEP 2. MS: m/z Calc'd for C₁₉H₂₀ClFN₂O₃[M+H]⁺379. Found, 379. ¹H NMR (400 MHz, Methanol-d₄) δ 7.42-7.32 (m, 4H), 7.28 (d, J=8.5 Hz, 2H), 7.13-7.07 (m, 2H), 4.96 (d, J=3.5 Hz, 1H), 4.40 (d, J=4.1 Hz, 1H), 4.37-4.26 (m, 2H), 4.23-4.19 (m, 1H), 3.59 (dd, J=12.6, 4.3 Hz, 1H), 3.24 (d, J=12.6 Hz, 1H), 3.16 (dd, J=14.0, 7.4 Hz, 1H), 3.04 (dd, J=14.0, 8.3 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 18% B to 48% B in 10 min; Wave Length: 254 nm/220 nm; RT1(min): 8.87.

### Synthetic Procedure of (2R,3S,4S)-2-[(4-chlorophenyl)methyl]-4-hydroxypyrrolidin-3-yl N-[(3,5-difluorophenyl)methyl]carbamate (V188272)

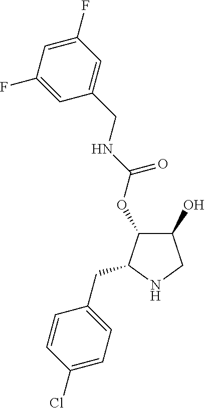 [SMILES: O=C(NCc1cc(F)cc(F)c1)O[C@@H]1[C@@H](O)CN[C@@H]1Cc1ccc(Cl)cc1]

The title compound was prepared in 54.9% overall yield as a white solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3,5-difluorophenyl)methanamine in STEP 1; Boc Deprotection; General Procedure II in STEP 2. MS: m/z Calc'd for C₁₉H₁₉ClF₂N₂O₃ [M+H]⁺397. Found, 397. ¹H NMR (400 MHz, Methanol-d₄) δ 7.35 (dd, J=8.5, 1.9 Hz, 2H), 7.30 (d, J=8.4 Hz, 2H), 7.04-6.94 (m, 2H), 6.90-6.86 (m, 1H), 4.95 (d, J=3.5 Hz, 1H), 4.41 (d, J=4.3 Hz, 1H), 4.39-4.27 (m, 2H), 4.24-4.20 (m, 1H), 3.61 (dd, J=12.5, 4.3 Hz, 1H), 3.25 (d, J=12.7 Hz, 1H), 3.18 (dd, J=14.0, 7.5 Hz, 1H), 3.07 (dd, J=14.1, 8.1 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 19% B to 49% B in 10 min; Wave Length: 254 nm/220 nm; RT1(min): 8.98.

### Synthetic Procedure of (2R,3S,4S)-2-[(4-chlorophenyl)methyl]-4-hydroxypyrrolidin-3-yl N-[(4-chlorophenyl)methyl]carbamate (V188273)

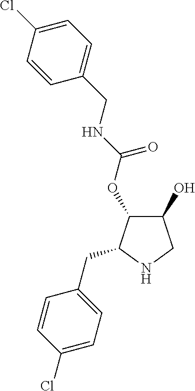 [SMILES: O=C(NCc1ccc(Cl)cc1)O[C@@H]1[C@@H](O)CN[C@@H]1Cc1ccc(Cl)cc1]

The title compound was prepared in 54.9% overall yield as a white solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(4-chlorophenyl)methanamine in STEP 1; Boc Deprotection; General Procedure II in STEP 2. MS: m/z Calc'd for C₁₉H₂₀Cl₂N₂O₃ [M+H]⁺395. Found, 395. ¹H NMR (400 MHz, Methanol-d₄) δ 7.41-7.36 (m, 2H), 7.35-7.30 (m, 4H), 7.28 (d, J=8.5 Hz, 2H), 4.96 (d, J=3.5 Hz, 1H), 4.40 (d, J=4.2 Hz, 1H), 4.39-4.27 (m, 2H), 4.24-4.20 (m, 1H), 3.59 (dd, J=12.6, 4.3 Hz, 1H), 3.24 (d, J=12.6 Hz, 1H), 3.16 (dd, J=14.0, 7.5 Hz, 1H), 3.04 (dd, J=14.1, 8.3 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 20% B to 50% B in 10 min; Wave Length: 254 nm/220 nm; RT1(min): 9.1.

### Synthetic Procedure of (2R,3S,4S)-2-[(4-chlorophenyl)methyl]-4-hydroxypyrrolidin-3-yl N-[(2-oxo-1H-pyridin-4-yl)methyl]carbamate (V188274)

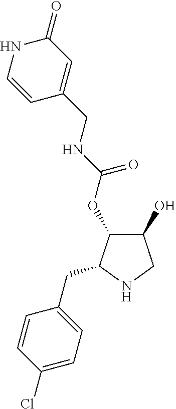 [SMILES: O=C(NCc1cc[nH]c(=O)c1)O[C@@H]1[C@@H](O)CN[C@@H]1Cc1ccc(Cl)cc1]

The title compound was prepared in 26.1% overall yield as a green solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 4-(aminomethyl)-1H-pyridin-2-one in STEP 1; Boc Deprotection; General Procedure II in STEP 2. MS: m/z Calc'd for C₁₈H₂₀ClN₃O₄[M+H]⁺378. found 378. ¹H NMR (400 MHz, Methanol-d₄) δ 7.57-7.27 (m, 5H), 6.46 (s, 1H), 6.37 (dd, J=6.8, 1.7 Hz, 1H), 4.99 (d, J=3.6 Hz, 1H), 4.43 (d, J=4.1 Hz, 1H), 4.38-4.27 (m, 1H), 4.27-4.18 (m, 3H), 3.62 (dd, J=12.6, 4.3 Hz, 1H), 3.26 (d, J=12.6 Hz, 1H), 3.20 (dd, J=14.2, 7.1 Hz, 1H), 3.06 (dd, J=14.1, 8.5 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 29% B in 10 min; Wave Length: 254 nm/220 nm; RT1(min): 9.9.

### Synthetic Procedure of (2R,3S,4S)-2-[(4-chlorophenyl)methyl]-4-hydroxypyrrolidin-3-yl N-({4-[(trifluoromethyl)sulfanyl]phenyl}methyl)carbamate (V188285)

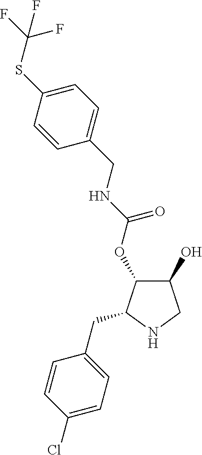 [SMILES: O=C(NCc1ccc(SC(F)(F)F)cc1)O[C@@H]1[C@@H](O)CN[C@@H]1Cc1ccc(Cl)cc1]

The title compound was prepared in 51.7% overall yield as a white solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-{4-[(trifluoromethyl)sulfanyl]phenyl}methanamine in STEP 1; Boc Deprotection; General Procedure II in STEP 2. MS: m/z Calc'd for C₂₀H₂₀ClF₃N₂O₃S [M+H]⁺461. Found, 461. ¹H NMR (400 MHz, Methanol-d₄) δ 7.70 (d, J=7.9 Hz, 2H), 7.47 (d, J=7.9 Hz, 2H), 7.31-7.29 (m, 4H), 4.96 (d, J=3.6 Hz, 1H), 4.52-4.33 (m, 3H), 4.27-4.21 (m, 1H), 3.62-3.58 (m, 1H), 3.25 (d, J=12.6 Hz, 1H), 3.17 (dd, J=14.1, 7.4 Hz, 1H), 3.09-3.03 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 26% B to 56% B in 10 min; Wave Length: 254 nm/220 nm; RT1(min): 9.06.

### Synthetic Procedure of (2R,3S,4S)-2-[(4-chlorophenyl)methyl]-4-hydroxypyrrolidin-3-yl N-[(3R)-oxolan-3-ylmethyl]carbamate (V188287)

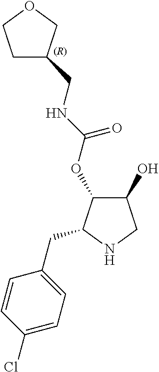

The title compound was prepared in 33.1% overall yield as a white solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-[(3R)-oxolan-3-yl]methanamine in STEP 1; Boc Deprotection; General Procedure II in STEP 2. MS: m/z Calc'd for C₁₇H₂₃ClN₂O₄[M+H]⁺355. Found, 355. ¹H NMR (400 MHz, Methanol-d₄) δ 7.43-7.29 (m, 4H), 4.94 (d, J=3.5 Hz, 1H), 4.39 (d, J=4.2 Hz, 1H), 4.25-4.19 (m, 1H), 3.94-3.71 (m, 3H), 3.66-3.52 (m, 2H), 3.28-2.99 (m, 5H), 2.52-2.46 (m, 1H), 2.14-2.01 (m, 1H), 1.72-1.64 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 6% B to 36% B in 10 min; Wave Length: 254 nm/220 nm; RT1(min): 9.62.

### Synthetic Procedure of (2R,3S,4S)-2-[(4-chlorophenyl)methyl]-4-hydroxypyrrolidin-3-yl N-(1,2-oxazol-4-ylmethyl)carbamate (V188289)

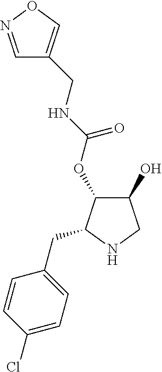 [SMILES: O=C(NCc1cnoc1)O[C@@H]1[C@@H](O)CN[C@@H]1Cc1ccc(Cl)cc1]

The title compound was prepared in 30.3% overall yield as a pink solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(1,2-oxazol-4-yl)methanamine in STEP 1; Boc Deprotection; General Procedure II in STEP 2. MS: m/z Calc'd for C₁₆H₁₈ClN₃O₄[M+H]⁺352. found 352. ¹H NMR (400 MHz, Methanol-d₄) δ 8.65 (s, 1H), 8.43 (s, 1H), 7.41-7.24 (m, 4H), 4.97 (d, J=3.6 Hz, 1H), 4.42-4.36 (m, 1H), 4.22 (dd, J=11.6, 3.6 Hz, 3H), 3.57 (dd, J=12.6, 4.3 Hz, 1H), 3.23 (d, J=12.6 Hz, 1H), 3.15 (dd, J=14.1, 7.3 Hz, 1H), 3.02 (dd, J=14.1, 8.4 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 8% B to 38% B in 10 min; Wave Length: 254 nm/220 nm; RT1(min): 9.33.

### Synthetic Procedure of (2R,3S,4S)-2-[(4-chlorophenyl)methyl]-4-hydroxypyrrolidin-3-yl N-[(4-sulfamoylphenyl)methyl]carbamate (V188291)

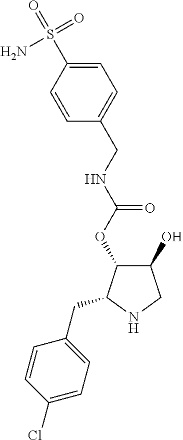 [SMILES: NS(=O)(=O)c1ccc(CNC(=O)O[C@@H]2[C@@H](O)CN[C@@H]2Cc2ccc(Cl)cc2)cc1]

The title compound was prepared in 30.3% overall yield as a white solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), emilene in STEP 1; Boc Deprotection; General Procedure II in STEP 2. MS: m/z Calc'd for C₁₉H₂₂ClN₃O₅S [M+H]⁺440. found 440. ¹H NMR (400 MHz, Methanol-d₄) δ 7.94-7.87 (m, 2H), 7.50 (d, J=8.3 Hz, 2H), 7.39-7.33 (m, 2H), 7.30 (d, J=8.5 Hz, 2H), 4.98 (d, J=3.5 Hz, 1H), 4.52-4.35 (m, 3H), 4.30-4.18 (m, 1H), 3.59-3.35 (m, 1H), 3.24 (d, J=12.6 Hz, 1H), 3.22-3.12 (m, 1H), 3.04-2.82 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 6% B to 36% B in 10 min; Wave Length: 254 nm/220 nm; RT1(min): 9.83.

### Synthetic Procedure of (2R,3S,4S)-4-hydroxy-2-[(4-methoxyphenyl)methyl]pyrrolidin-3-yl N-({3-aminobicyclo[1.1.1]pentan-1-yl}methyl)carbamate (V188323)

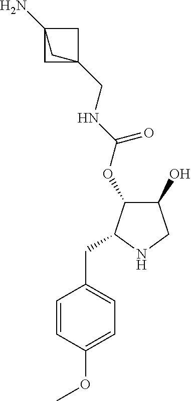 [SMILES: COc1ccc(C[C@H]2NC[C@H](O)[C@H]2OC(=O)NCC23CC(N)(C2)C3)cc1]

The title compound was prepared in 51.1% overall yield as a white solid according to Substitution reaction for synthesis of Carbamate; General Procedure I using tert-butyl (3-(aminomethyl)bicyclo[1.1.1]pentan-1-yl)carbamate in STEP 1; Boc Deprotection; General Procedure II in STEP 2. MS: m/z Calc'd for C₁₉H₂₇N₃O₄ [M+H]⁺362. Found, 362. ¹H NMR (400 MHz, Methanol-d₄) δ 7.28-7.20 (m, 2H), 6.98-6.90 (m, 2H), 4.95 (d, J=3.5 Hz, 1H), 4.38 (d, J=4.5 Hz, 1H), 4.21-4.11 (m, 1H), 3.80 (s, 3H), 3.64-3.53 (m, 1H), 3.44 (d, J=14.7 Hz, 1H), 3.22 (d, J=12.6 Hz, 1H), 3.15-3.07 (m, 1H), 2.97 (dd, J=13.6, 7.9 Hz, 1H), 2.09-1.96 (m, 6H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 19% B in 10 min; Wave Length: 254 nm/220 nm; RT1(min): 9.1.

### Synthetic Procedure of (2R,3S,4S)-4-hydroxy-2-[(4-methoxyphenyl)methyl]pyrrolidin-3-yl N-{bicyclo[1.1.1]pentan-1-ylmethyl}carbamate (V188324)

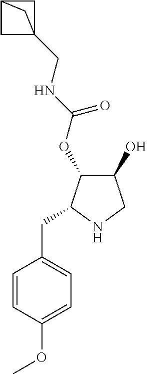 [SMILES: COc1ccc(C[C@H]2NC[C@H](O)[C@H]2OC(=O)NCC23CC(C2)C3)cc1]

The title compound was prepared in 31.2% overall yield as a white solid according to Substitution reaction for synthesis of Carbamate; General Procedure I using 1-{bicyclo[1.1.1]pentan-1-yl}methanamine in STEP 1; Boc Deprotection; General Procedure II in STEP 2. MS: m/z Calc'd for C₁₉H₂₆N₂O₄ [M+H]⁺347. Found, 347. ¹H NMR (400 MHz, Methanol-d₄) δ 7.27-7.20 (m, 2H), 6.97-6.90 (m, 2H), 4.93 (d, J=3.5 Hz, 1H), 4.38 (d, J=4.1 Hz, 1H), 4.18-4.14 (m, 1H), 3.80 (s, 3H), 3.62-3.52 (m, 1H), 3.26-3.18 (m, 2H), 3.15-3.10 (m, 2H), 2.96 (dd, J=14.1, 8.3 Hz, 1H), 2.52 (s, 1H), 1.77 (s, 6H).

Prep-HPLC purification conditions: Column: Welch Ultimate XB-Phenyl 30*250, 10 μm; Mobile Phase A: Water (0.05% TFA), Mobile Phase B: ACN; Flow rate: 60 mL/min; Gradient: 20% B to 50% B in 10 min; Wave Length: 254 nm/220 nm; RT1(min): 7.5.

### Synthetic Procedure of (2R,3S,4S)-4-hydroxy-2-[(4-methoxyphenyl)methyl]pyrrolidin-3-yl N-[(4-fluorophenyl)methyl]carbamate (V188378)

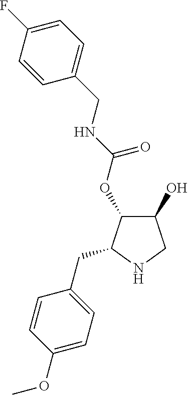 [SMILES: COc1ccc(C[C@H]2NC[C@H](O)[C@H]2OC(=O)NCc2ccc(F)cc2)cc1]

The title compound was prepared in 33.9% overall yield as a white solid according to Substitution reaction for synthesis of Carbamate; General Procedure I using 4-fluorobenzylamine in STEP 1; Boc Deprotection; General Procedure II in STEP 2. MS: m/z Calc'd for C₂₀H₂₃FN₂O₄ [M+H]⁺375. Found, 375. ¹H NMR (400 MHz, Methanol-d₄) δ 7.38-7.32 (m, 2H), 7.17 (d, J=8.2 Hz, 2H), 7.13-7.03 (m, 2H), 6.89-6.81 (m, 2H), 4.91 (s, 1H), 4.39-4.23 (m, 3H), 4.17-4.11 (m, 1H), 3.77 (d, J=1.6 Hz, 3H), 3.57 (dd, J=12.7, 4.5 Hz, 1H), 3.20 (d, J=12.6 Hz, 1H), 3.08 (dd, J=14.1, 7.7 Hz, 1H), 2.96 (dd, J=13.7, 7.2 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 15% B to 45% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-[(3,5-difluorophenyl)methyl]carbamate (V188380)

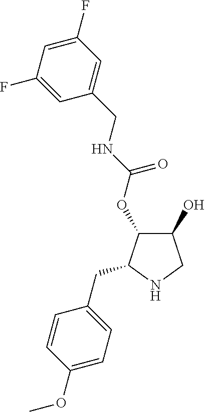 [SMILES: COc1ccc(C[C@H]2NC[C@H](O)[C@H]2OC(=O)NCc2cc(F)cc(F)c2)cc1]

The title compound was prepared in 33.9% overall yield as a white solid according to Substitution reaction for synthesis of Carbamate; General Procedure I using 1-(3,5-difluorophenyl)methanamine in STEP 1; Boc Deprotection; General Procedure II in STEP 2. MS: m/z Calc'd for C₂₀H₂₂F₂N₂O₄[M+H]⁺393. Found, 393. ¹H NMR (400 MHz, Methanol-d₄) δ 7.23-7.15 (m, 2H), 7.00-6.91 (m, 2H), 6.90-6.81 (m, 3H), 4.90 (d, J=3.5 Hz, 1H), 4.46-4.34 (m, 2H), 4.28 (d, J=15.8 Hz, 1H), 4.18-4.14 (m, 1H), 3.77 (s, 3H), 3.62-3.52 (m, 1H), 3.22 (d, J=12.6 Hz, 1H), 3.10 (dd, J=14.0, 7.8 Hz, 1H), 2.98 (dd, J=13.9, 8.0 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 15% B to 45% B in 10 min; Wave Length: 254 nm/220 nm; RT1(min): 8.9.

### Synthetic Procedure of (2R,3S,4S)-4-hydroxy-2-[(4-methoxyphenyl)methyl]pyrrolidin-3-yl N-(1,2-thiazol-4-ylmethyl)carbamate (V188384)

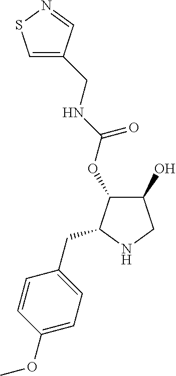 [SMILES: COc1ccc(C[C@H]2NC[C@H](O)[C@H]2OC(=O)NCc2cnsc2)cc1]

The title compound was prepared in 31.5% overall yield as a white solid according to Substitution reaction for synthesis of Carbamate; General Procedure I using 1-(1,2-thiazol-4-yl)methanamine in STEP 1; Boc Deprotection; General Procedure II in STEP 2. MS: m/z Calc'd for C₁₇H₂₁N₃O₄S [M+H]⁺364. Found, 364. ¹H NMR (400 MHz, Methanol-d₄) δ 8.79 (s, 1H), 8.53 (s, 1H), 7.22-7.15 (m, 2H), 6.92-6.84 (m, 2H), 4.94 (d, J=3.5 Hz, 1H), 4.53-4.36 (m, 3H), 4.19-4.15 (m, 1H), 3.80 (s, 3H), 3.57 (dd, J=12.6, 4.3 Hz, 1H), 3.23 (d, J=12.6 Hz, 1H), 3.09 (dd, J=14.0, 7.6 Hz, 1H), 2.96 (dd, J=14.0, 8.2 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 4% B to 34% B in 10 min; Wave Length: 254 nm/220 nm; RT1(min): 9.1.

### Synthetic Procedure of (2R,3S,4S)-4-hydroxy-2-[(4-methoxyphenyl)methyl]pyrrolidin-3-yl 2-[(2S)-pyrrolidin-2-yl]ethyl carbonate (V191942)

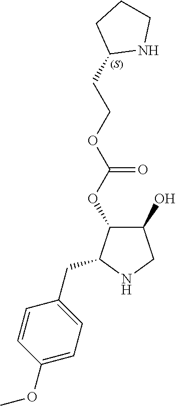

The title compound was prepared in 46.7% overall yield as a white solid according to Substitution reaction for synthesis of Carbamate; General Procedure I using tert-butyl (2S)-2-(2-hydroxyethyl)pyrrolidine-1-carboxylate in STEP 1; Boc Deprotection; General Procedure II in STEP 2. MS: m/z Calc'd for C₁₉H₂₈N₂O₅ [M+H]⁺365. Found, 365. ¹H NMR (400 MHz, Methanol-d₄) δ 7.29-7.21 (m, 2H), 6.98-6.90 (m, 2H), 4.99 (d, J=3.8 Hz, 1H), 4.49 (d, J=4.3 Hz, 1H), 4.41-4.31 (m, 2H), 4.26-4.20 (m, 1H), 3.80 (s, 3H), 3.71-3.58 (m, 2H), 3.37 (dd, J=7.8, 4.0 Hz, 2H), 3.24 (d, J=12.8 Hz, 1H), 3.14 (dd, J=14.3, 6.5 Hz, 1H), 3.06-2.95 (m, 1H), 2.38-1.97 (m, 5H), 1.80-1.70 (m, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 30% B in 15 min; Wave Length: 254 nm/220 nm; RT1(min): 16.5.

### Synthetic Procedure of (2R,3S,4S)-4-hydroxy-2-[(4-methoxyphenyl)methyl]pyrrolidin-3-yl N-[(1S)-1-phenylethyl]carbamate (V191971)

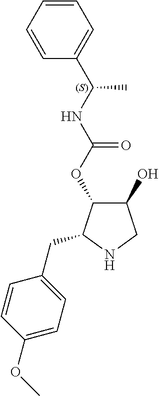

The title compound was prepared in 33.2% overall yield as a white solid according to Substitution reaction for synthesis of Carbamate; General Procedure I using (S)-a-phenylethylamine in STEP 1; Boc Deprotection; General Procedure II in STEP 2. MS: m/z Calc'd for C₂₁H₂₆N₂O₄ [M+H]⁺371. Found, 371. ¹H NMR (400 MHz, Methanol-d₄) δ 7.44-7.34 (m, 4H), 7.33-7.23 (m, 1H), 7.10 (d, J=8.2 Hz, 2H), 6.74 (d, J=8.1 Hz, 2H), 4.85-4.72 (m, 2H), 4.38 (d, J=4.1 Hz, 1H), 4.15-4.10 (m, J=8.0, 3.6 Hz, 1H), 3.74 (s, 3H), 3.59 (dd, J=12.7, 4.0 Hz, 1H), 3.24 (d, J=12.6 Hz, 1H), 3.04 (dd, J=13.8, 8.3 Hz, 1H), 2.98-2.91 (m, 1H), 1.50 (d, J=7.1 Hz, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-[(2,6-difluorophenyl)methyl]carbamate (V190521)

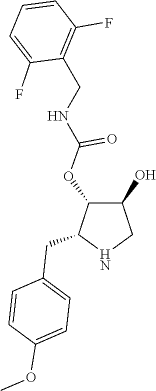 [SMILES: COc1ccc(C[C@H]2NC[C@H](O)[C@H]2OC(=O)NCc2c(F)cccc2F)cc1]

The title compound was prepared in 34.9% overall yield as a white solid according to Substitution reaction for synthesis of Carbamate; General Procedure I using 1-(2,6-difluorophenyl)methanamine in STEP 1; Boc Deprotection; General Procedure II in STEP 2. MS: m/z Calc'd for C₂₀H₂₂F₂N₂O₄[M+H]⁺393. Found, 393. ¹H NMR (400 MHz, Methanol-d₄) δ 7.44-7.36 (m, 1H), 7.17 (d, J=8.2 Hz, 2H), 7.06-7.00 (m, 2H), 6.89-6.83 (m, 2H), 4.58-4.35 (m, 3H), 4.16-4.11 (m, 1H), 3.78 (s, 3H), 3.58-3.53 (m, 1H), 3.21 (d, J=12.7 Hz, 1H), 3.07 (dd, J=14.0, 7.6 Hz, 1H), 2.99-2.88 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 7% B to 37% B in 10 min; Wave Length: 254 nm/220 nm; RT1(min): 10.17.

### Synthetic Procedure of (2R,3S,4S)-4-hydroxy-2-{[4-(1,3-thiazol-5-yl)phenyl]methyl}pyrrolidin-3-yl N-[2-(piperidin-4-yl)ethyl]carbamate (V192112)

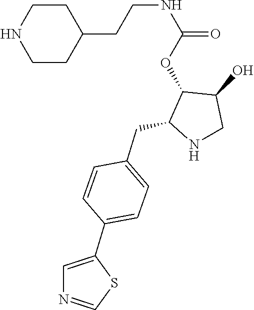 [SMILES: O=C(NCCC1CCNCC1)O[C@@H]1[C@@H](O)CN[C@@H]1Cc1ccc(-c2cncs2)cc1]

The title compound was prepared in 40.1% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-thiazol-5-yl)phenyl]methyl}pyrrolidine-1-carboxylate (Int-13) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 4-(2-aminoethyl)piperidine-1-carboxylate in STEP 1; Boc Deprotection; General Procedure II in STEP 2. MS: m/z Calc'd for C₂₄H₃₁N₃O₃ [M+H]⁺410. found 410. ¹H NMR (400 MHz, Methanol-d₄) δ 9.00 (s, 1H), 8.19 (s, 1H), 7.73-7.66 (m, 2H), 7.50-7.40 (m, 2H), 5.04-4.98 (m, 1H), 4.43-4.38 (m, 1H), 4.27 (ddd, J=10.8, 7.3, 3.5 Hz, 1H), 3.59 (dd, J=12.6, 4.3 Hz, 1H), 3.41 (d, J=12.9 Hz, 3H), 3.24 (dd, J=13.8, 9.4, 4.7 Hz, 3H), 3.09 (dd, J=14.1, 8.5 Hz, 1H), 2.98 (t, J=12.7 Hz, 2H), 2.02 (d, J=13.7 Hz, 2H), 1.76-1.62 (m, 1H), 1.57 (d, J=7.2 Hz, 2H), 1.44-1.41 (s, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 24% B in 13 min; Wave Length: 254 nm/220 nm; RT1(min): 11.5.

### Synthetic Procedure of (2R,3S,4S)-4-hydroxy-2-{[4-(1,3-oxazol-5-yl)phenyl]methyl}pyrrolidin-3-yl N-{2-[(3S,4R)-3-fluoropiperidin-4-yl]ethyl}carbamate (V192897)

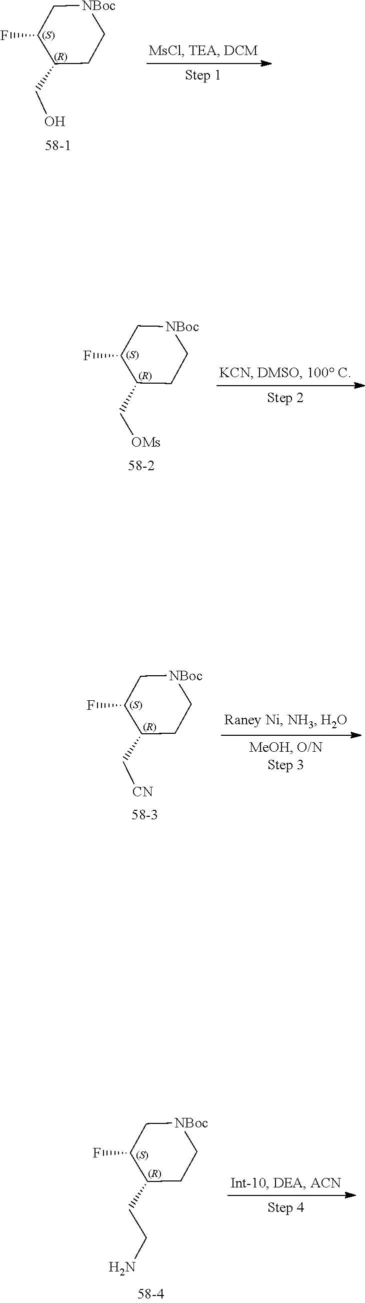

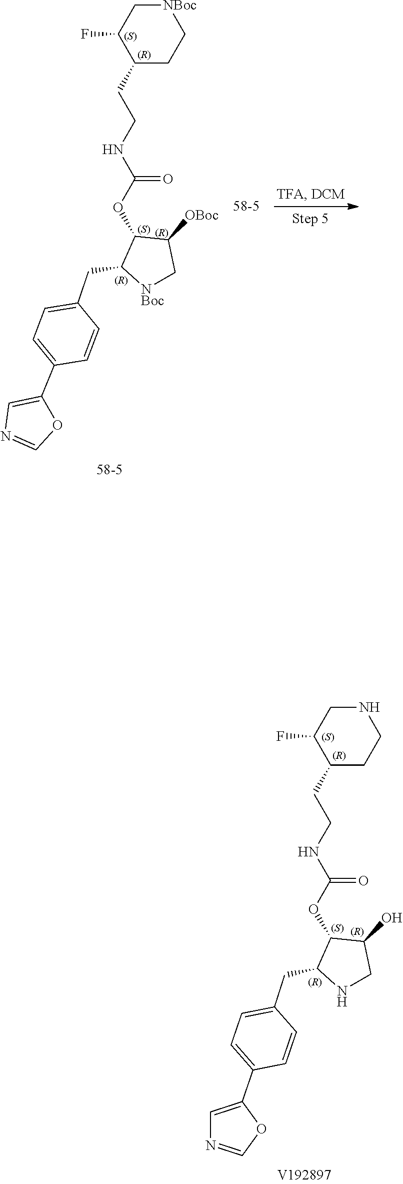

Step 1: To a solution of tert-butyl (3S,4R)-3-fluoro-4-(hydroxymethyl)piperidine-1-carboxylate (500 mg, 2.14 mmol, 1 equiv) in DCM (10 mL) was added Et₃N (650.6 mg, 6.42 mmol, 3 equiv) and methanesulfonyl chloride (490.9 mg, 4.28 mmol, 2 equiv). After stirring for overnight at room temperature under a nitrogen atmosphere, the resulting mixture was concentrated under reduced pressure. The residue was purified by silica gel column chromatography, eluted with PE/EA (2:1) to afford tert-butyl (3S,4R)-3-fluoro-4-[(methanesulfonyloxy)methyl]piperidine-1-carboxylate (700 mg) as a off-white solid. MS: m/z: Calc'd for C₁₂H₂₂FNO₅S [M−56]⁺256. found 256.

Step 2: To a solution of tert-butyl (3S,4R)-3-fluoro-4-[(methanesulfonyloxy)methyl]piperidine-1-carboxylate (690 mg, 2.21 mmol, 1 equiv) in DMSO (10 mL) was added KCN (288.6 mg, 4.43 mmol, 2 equiv). After stirring for overnight at 100° C. under a nitrogen atmosphere, the resulting mixture was concentrated under reduced pressure. The residue was purified by silica gel column chromatography, eluted with PE/EA (2:1) to afford tert-butyl (3S,4R)-4-(cyanomethyl)-3-fluoropiperidine-1-carboxylate (500 mg) as a off-white solid. MS: m/z: Calc'd for C₁₂H₁₉FN₂O₂[M−56]⁺ 187. found 187.

Step 3: To a solution of tert-butyl (3S,4R)-4-(cyanomethyl)-3-fluoropiperidine-1-carboxylate (240 mg, 0.99 mmol, 1 equiv) in methanol (5 mL) and Ammonium hydroxide (28% in water) (0.2 mL) was added raney nickel (116.2 mg, 1.98 mmol, 2 equiv) in a pressure tank. The mixture was hydrogenated at room temperature under 20 psi of hydrogen pressure for overnight, filtered through a Celite pad and concentrated under reduced pressure to afford tert-butyl (3S,4S)-4-(2-aminoethyl)-3-fluoropiperidine-1-carboxylate (220 mg) as a light yellow solid. MS: m/z: Calc'd for C₁₂H₂₃FN₂O₂[M+H]⁺ 247. found 247.

### Step 4 and 5

The title compound was prepared in 38.6% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (3S,4S)-4-(2-aminoethyl)-3-fluoropiperidine-1-carboxylate (58-4) in STEP 4; Boc Deprotection; General Procedure II in STEP 5. MS: m/z Calc'd for C₂₂H₂₉FN₄O₄[M+H]⁺433. found 433. ¹H NMR (400 MHz, Methanol-d₄) δ 8.27 (s, 1H), 7.75 (dd, J=8.2, 2.4 Hz, 2H), 7.54 (s, 1H), 7.45 (d, J=8.0 Hz, 2H), 4.98 (t, J=23.2 Hz, 2H), 4.39 (d, J=4.2 Hz, 1H), 4.26 (d, J=9.1 Hz, 1H), 3.71-3.56 (m, 2H), 3.41 (d, J=12.9 Hz, 1H), 3.33-3.17 (m, 5H), 3.18-2.95 (m, 2H), 2.14-1.71 (m, 2H), 1.64 (dd, J=13.8, 7.0 Hz, 1H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-{2-[(2R,4S)-4-fluoropyrrolidin-2-yl]ethyl}carbamate (V192917)

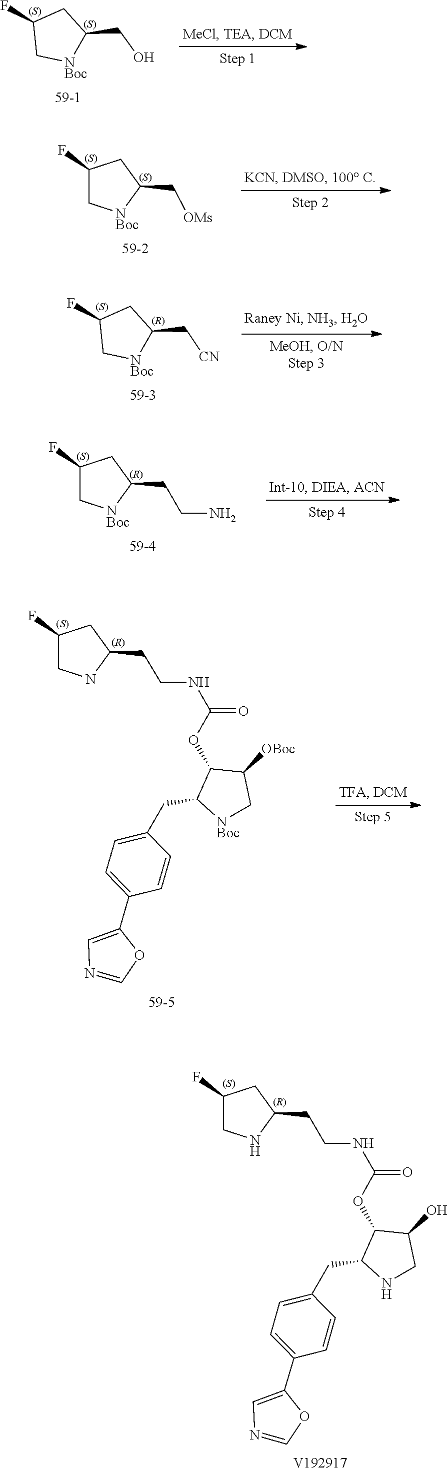

Step 1: To a solution of tert-butyl (2S,4S)-4-fluoro-2-(hydroxymethyl)pyrrolidine-1-carboxylate (500 mg, 2.28 mmol, 1 equiv) in DCM (10 mL) was added Et₃N (692.2 mg, 6.84 mmol, 3 equiv) and methanesulfonyl chloride (522.4 mg, 4.56 mmol, 2 equiv). After stirring for overnight at room temperature under a nitrogen atmosphere, the resulting mixture was concentrated under reduced pressure. The residue was purified by silica gel column chromatography, eluted with PE/EA (2:1) to afford tert-butyl (2S,4S)-4-fluoro-2-[(methanesulfonyloxy)methyl]pyrrolidine-1-carboxylate (700 mg) as an off-white oil. MS: m/z: Calc'd for C₁₁H₂₀FNO₅S [M−100]⁺198. found 198.

Step 2: To a solution of tert-butyl (2S,4S)-4-fluoro-2-[(methanesulfonyloxy)methyl]pyrrolidine-1-carboxylate (690 mg, 2.32 mmol, 1 equiv) in DMSO (10 mL) was added KCN (302.2 mg, 4.64 mmol, 2 equiv). After stirring for overnight at 100° C. under a nitrogen atmosphere, the resulting mixture was concentrated under reduced pressure. The residue was purified by silica gel column chromatography, eluted with PE/EA (2:1) to afford tert-butyl (2R,4S)-2-(cyanomethyl)-4-fluoropyrrolidine-1-carboxylate (250 mg) as an off-white oil. MS: m/z: Calc'd for C₁₁H₁₇FN₂O₂[M+H]⁺229. found 229.

Step 3: To a solution of tert-butyl (2R,4S)-2-(cyanomethyl)-4-fluoropyrrolidine-1-carboxylate (240 mg, 1.05 mmol, 1 equiv) in methanol (5 mL) and Ammonium hydroxide (28% in water) (0.2 mL) was added raney nickel (123.42 mg, 2.102 mmol, 2 equiv) in a pressure tank. The mixture was hydrogenated at room temperature under 20 psi of hydrogen pressure for overnight, filtered through a Celite pad and concentrated under reduced pressure to afford tert-butyl (2R,4S)-2-(2-aminoethyl)-4-fluoropyrrolidine-1-carboxylate (200 mg) as an off-white oil. MS: m/z: Calc'd for C₁₁H₂₁FN₂O₂[M+H]⁺233. found 233.

Step 4 and 5: The title compound was prepared in 38.6% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2R,4S)-2-(2-aminoethyl)-4-fluoropyrrolidine-1-carboxylate (59-4) in STEP 4; Boc Deprotection; General Procedure II in STEP 5. MS: m/z Calc'd for C₂₁H₂₇FN₄O₄[M+H]⁺419. found 419. ¹H NMR (400 MHz, Methanol-d₄) δ 8.27 (s, 1H), 7.76 (d, J=7.8 Hz, 2H), 7.54 (s, 1H), 7.45 (d, J=8.0 Hz, 2H), 5.44 (d, J=52.8 Hz, 1H), 5.01 (s, 1H), 4.41 (d, J=4.2 Hz, 1H), 4.26 (s, 1H), 3.87-3.75 (m, 1H), 3.75-3.64 (m, 1H), 3.61 (dt, J=12.5, 2.6 Hz, 1H), 3.53-3.36 (m, 2H), 3.31 (d, J=6.4 Hz, 1H), 3.23 (dd, J=13.7, 7.8 Hz, 2H), 3.12 (dd, J=14.3, 8.6 Hz, 1H), 2.77-2.57 (m, 1H), 2.22-1.96 (m, 3H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.5.

### Synthetic Procedure of (2R,3S,4S)-2-[(4-cyclopropylphenyl)methyl]-4-hydroxypyrrolidin-3-yl N-benzylcarbamate (V188329)

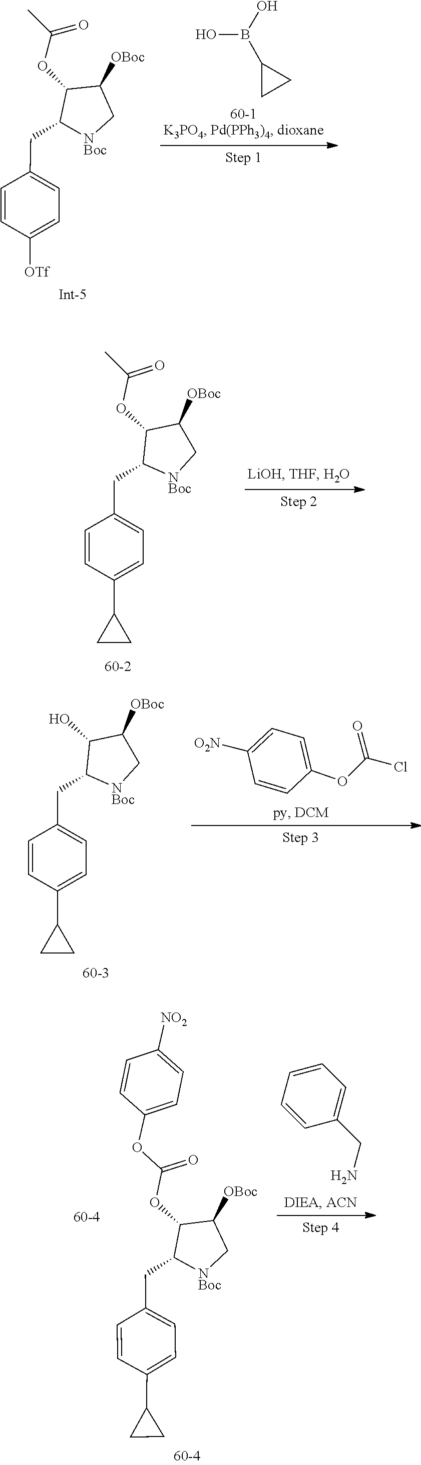

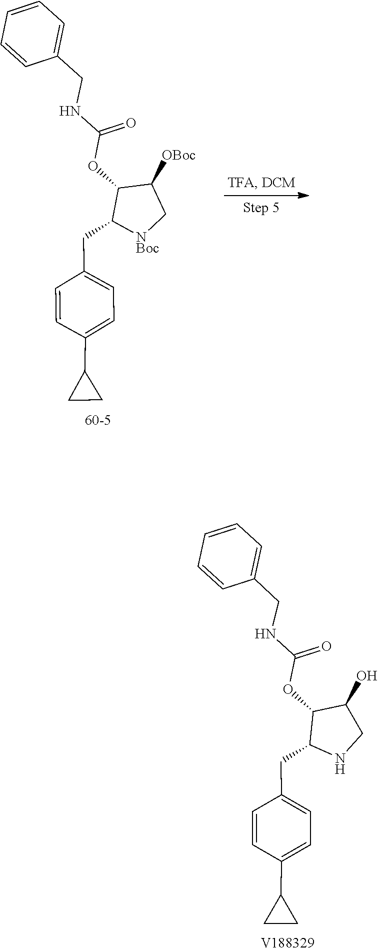

Step 1: To a stirred solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (400 mg, 0.69 mmol, 1.0 eq.) and cyclopropylboronic acid (76.5 mg, 0.89 mmol, 1.3 eq.) in dioxane (10 mL) was added K₃PO₄ (291 mg, 1.37 mmol, 2.0 eq.) and Pd(PPh₃)₄ (39.6 mg, 0.03 mmol, 0.05 eq.) in portions at room temperature. The resulting mixture was stirred over night at 100° C. under a nitrogen atmosphere. Desired product could be detected by LCMS. The resulting mixture was filtered, the filter cake was washed with dioxane (10 mL) (3×3 mL). The filtrate was concentrated under reduced pressure. The residue was purified by reversed-phase flash chromatography to afford tert-butyl (2R,3S,4S)-3-(acetyloxy)-4-[(tert-butoxycarbonyl)oxy]-2-[(4-cyclopropylphenyl)methyl]pyrrolidine-1-carboxylate (250 mg, 76.7%) as a light yellow oil. MS: m/z: Calc'd for C₂₆H₃₇NO₇ [M+H−56−56]⁺364. Found, 364.

Step 2: To a stirred solution of tert-butyl (2R,3S,4S)-3-(acetyloxy)-4-[(tert-butoxycarbonyl)oxy]-2-[(4-cyclopropylphenyl)methyl]pyrrolidine-1-carboxylate (250 mg, 0.53 mmol, 1.0 eq.) in THF (5 mL) and H₂O (0.5 mL) was added LiOH (37.8 mg, 1.58 mmol, 3.0 eq.) in portions at room temperature. The resulting mixture was stirred over night at 25° C. The resulting mixture was extracted with EtOAc (2×5 mL). The combined organic layers were washed with H₂O (3×3 mL), dried over anhydrous Na2SO4. After filtration, the filtrate was concentrated under reduced pressure. This resulted in tert-butyl (2R,3S,4S)-4-[(tert-butoxycarbonyl)oxy]-2-[(4-cyclopropylphenyl)methyl]-3-hydroxypyrrolidine-1-carboxylate (200 mg, 87.8%) as a light yellow oil. MS: m/z: Calc'd for C₂₄H₃₅NO₆ [M+H−56−56]⁺322. Found, 322.

Step 3: To a stirred solution of tert-butyl (2R,3S,4S)-4-[(tert-butoxycarbonyl)oxy]-2-[(4-cyclopropylphenyl)methyl]-3-hydroxypyrrolidine-1-carboxylate (190 mg, 0.44 mmol, 1.0 eq.) and 4-nitrophenyl carbonochloridate (132.5 mg, 0.66 mmol, 1.5 eq.) in DCM (5 mL) was added Pyridine (69.3 mg, 0.88 mmol, 2.0 eq.) in portions at room temperature. The resulting mixture was stirred 2 h at 25° C. Desired product could be detected by LCMS. The residue was purified by Prep-TLC (PE/EA 2:1) to afford tert-butyl (2R,3S,4S)-4-[(tert-butoxycarbonyl)oxy]-2-[(4-cyclopropylphenyl)methyl]-3-[(4-nitrophenoxycarbonyl)oxy]pyrrolidine-1-carboxylate (200 mg, 76.2%) as a light yellow oil. MS: m/z: Calc'd for C₃₁H₃₈N₂O₁₀ [M+H−56−56]⁺487. Found, 487.

### Step 4 and 5

The title compound was prepared in 34.3% overall yield as a white solid according to Substitution reaction for synthesis of Carbamate; General Procedure I using tert-butyl (2R,3S,4S)-4-[(tert-butoxycarbonyl)oxy]-2-[(4-cyclopropylphenyl)methyl]-3-[(4-nitrophenoxycarbonyl)oxy]pyrrolidine-1-carboxylate (60-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), benzylamine in STEP 4; Boc Deprotection; General Procedure II in STEP 5. MS: m/z Calc'd for C₂₂H₂₆N₂O₃ [M+H]⁺367. Found, 367. ¹H NMR (400 MHz, Methanol-d₄) δ 7.36 (d, J=6.7 Hz, 4H), 7.32-7.28 (m, 1H), 7.19-7.12 (m, 2H), 7.07-7.00 (m, 2H), 4.93 (d, J=3.6 Hz, 1H), 4.45-4.25 (m, 3H), 4.19-4.14 (m, 1H), 3.61-3.55 (m, 1H), 3.22 (d, J=12.6 Hz, 1H), 3.12 (dd, J=13.9, 7.6 Hz, 1H), 3.02-2.92 (m, 1H), 1.93-1.86 (m, 1H), 1.02-0.92 (m, 2H), 0.70-0.61 (m, 2H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 10.17.

### Synthetic Procedure of (2R,3S,4S)-2-[(4-cyclopropylphenyl)methyl]-4-hydroxypyrrolidin-3-yl N-[(3-fluorophenyl)methyl]carbamate (V190518)

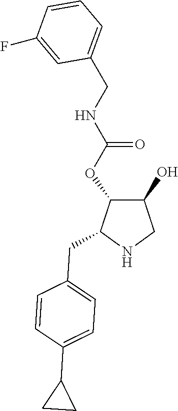 [SMILES: O=C(NCc1cccc(F)c1)O[C@@H]1[C@@H](O)CN[C@@H]1Cc1ccc(C2CC2)cc1]

The title compound was prepared in 37.9% overall yield as a white solid according to Substitution reaction for synthesis of Carbamate; General Procedure I using tert-butyl (2R,3S,4S)-4-[(tert-butoxycarbonyl)oxy]-2-[(4-cyclopropylphenyl)methyl]-3-[(4-nitrophenoxycarbonyl)oxy]pyrrolidine-1-carboxylate (60-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z Calc'd for C₂₂H₂₅FN₂O₃[M+H]⁺385. Found, 385. ¹H NMR (400 MHz, Methanol-d₄) δ 7.41-7.36 (m, 1H), 7.20-6.99 (m, 7H), 4.44-4.26 (m, 3H), 4.20-4.15 (m, J=8.0, 3.5 Hz, 1H), 3.59 (dd, J=13.1, 4.1 Hz, 1H), 3.23 (d, J=12.7 Hz, 1H), 3.13 (dd, J=13.9, 7.7 Hz, 1H), 3.05-2.94 (m, 1H), 1.93-1.87 (m, 1H), 1.02-0.92 (m, 2H), 0.70-0.61 (m, 2H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 40% B to 70% B in 10 min; Wave Length: 254 nm/220 nm; RT1(min): 9.88.

### Synthetic Procedure of (2R,3S,4S)-2-{[4-(5-chlorothiophen-2-yl)phenyl]methyl}-4-hydroxypyrrolidin-3-yl N-{2-[(2S)-pyrrolidin-2-yl]ethyl}carbamate (V191891)

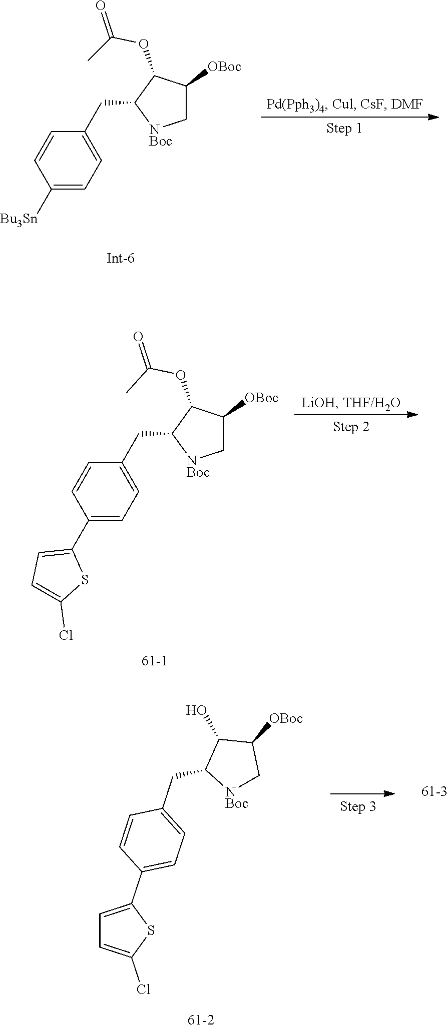

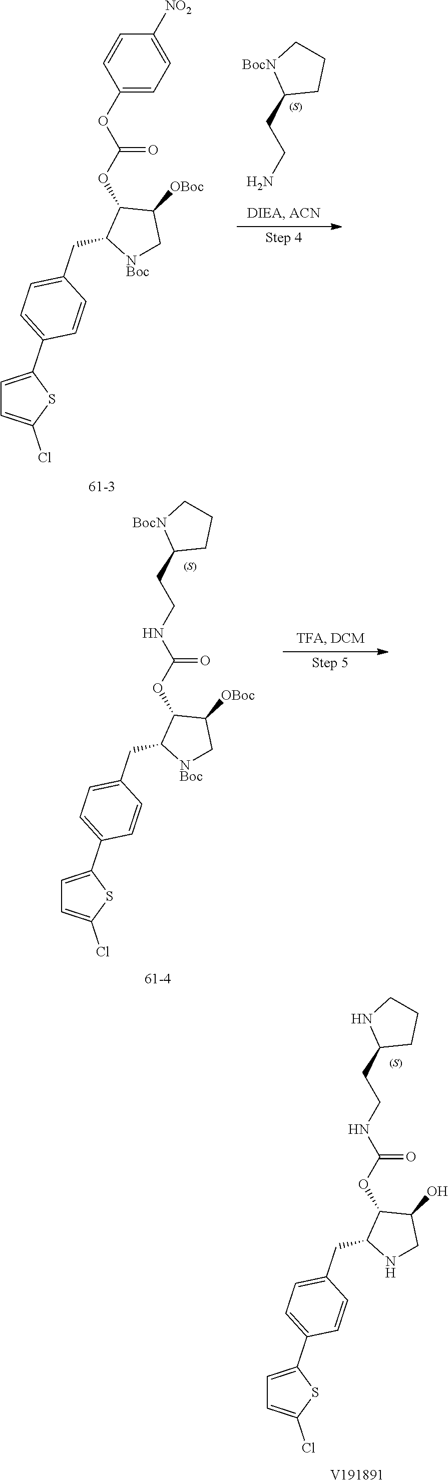

Step 1: To a stirred mixture of 2-bromo-5-chlorothiophene (163.5 mg, 0.82 mmol, 1.5 equiv) and tert-butyl (2R,3S,4S)-3-(acetyloxy)-4-[(tert-butoxycarbonyl)oxy]-2-{[4-(tributylstannyl)phenyl]methyl}pyrrolidine-1-carboxylate (400 mg, 0.55 mmol, 1.00 equiv) CuI (21.3 mg, 0.11 mmol, 0.2 equiv) in DMF (5 mL) was added Pd(PPh₃)₄ (63.7 mg, 0.05 mmol, 0.1 equiv) and CsF (167.2 mg, 1.14 mmol, 2 equiv) at room temperature under nitrogen atmosphere. The resulting mixture was stirred at 80° C. for overnight under nitrogen atmosphere. The residue was purified by reversed-phase flash chromatography with the following conditions: column, C18 silica gel; mobile phase, MeCN in Water (0.1% TFA), 0% to 100% gradient in 10 min; detector, UV 254 nm. This resulted in tert-butyl (2R,3S,4S)-3-(acetyloxy)-4-[(tert-butoxycarbonyl)oxy]-2-{[4-(5-chlorothiophen-2-yl)phenyl]methyl}pyrrolidine-1-carboxylate (300 mg, 98.4% yield, 85% purity) as a yellow solid. MS: m/z: Calc'd for C₂₇H₃₄ClNO₇S [M−100]⁺452. found 452.

Step 2: To a stirred mixture of tert-butyl (2R,3S,4S)-3-(acetyloxy)-4-[(tert-butoxycarbonyl)oxy]-2-{[4-(5-chlorothiophen-2-yl)phenyl]methyl}pyrrolidine-1-carboxylate (400 mg, 0.72 mmol, 1 equiv) in Water (1 mL) and THF (5 mL) was added LiOH (52.0 mg, 2.17 mmol, 3 equiv) at room temperature. The resulting mixture was stirred at room temperature for 2 h. The mixture neutralized to pH 7 with conc. HCl. The reaction mixture was quenched with water and extracted with EA. The extracts were washed with brine, dried over anhydrous sodium sulfate, filtered and concentrated. This resulted in tert-butyl (2R,3S,4S)-4-[(tert-butoxycarbonyl)oxy]-2-{[4-(5-chlorothiophen-2-yl)phenyl]methyl}-3-hydroxypyrrolidine-1-carboxylate (300 mg, 81.1% yield, 95% purity) as a white solid. MS: m/z: Calc'd for C₂₅H₃₂ClNO₆S [M+22]⁺ 532. found 532.

Step 3: To a stirred mixture of tert-butyl (2R,3S,4S)-4-[(tert-butoxycarbonyl)oxy]-2-{[4-(5-chlorothiophen-2-yl)phenyl]methyl}-3-hydroxypyrrolidine-1-carboxylate (620 mg, 1.21 mmol, 1 equiv) and py (288.4 mg, 3.64 mmol, 3 equiv) in DCM (10 mL) was added 4-nitrophenyl chloroformate (490.0 mg, 2.43 mmol, 2 equiv) at room temperature. The resulting mixture was stirred at room temperature for 2 h. The resulting mixture was concentrated under reduced pressure. The residue was dissolved in DCM (10 mL). The residue was purified by Prep-TLC (PE/EA 2:1) to afford tert-butyl (2R,3S,4S)-4-[(tert-butoxycarbonyl)oxy]-2-{[4-(5-chlorothiophen-2-yl)phenyl]methyl}-3-[(4-nitrophenoxycarbonyl)oxy]pyrrolidine-1-carboxylate (520 mg, 63.3% yield, 90% purity) as a yellow solid. MS: m/z: Calc'd for C₃₂H₃₅ClN₂O₁₀S [M+22]⁺697. found 697.

### Step 4 and 5

The title compound was prepared in 34.3% overall yield as a white solid according to Substitution reaction for synthesis of Carbamate; General Procedure I using tert-butyl (2R,3S,4S)-4-[(tert-butoxycarbonyl)oxy]-2-{[4-(5-chlorothiophen-2-yl)phenyl]methyl}-3-[(4-nitrophenoxycarbonyl)oxy]pyrrolidine-1-carboxylate (61-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate in STEP 4; Boc Deprotection; General Procedure II in STEP 5. MS: m/z C₂₂H₂₈ClN₃O₃S [M+H]⁺450. found 450. ¹H NMR (400 MHz, Methanol-d₄) δ 7.63-7.55 (m, 2H), 7.41-7.34 (m, 2H), 7.24 (d, J=3.9 Hz, 1H), 6.99 (d, J=3.9 Hz, 1H), 5.03-4.97 (m, 1H), 4.40 (d, J=4.1 Hz, 1H), 4.26 (ddd, J=8.6, 7.1, 3.6 Hz, 1H), 3.56-3.53 (m, 2H), 3.40 (s, 1H), 3.32-3.25 (m, 4H), 3.23-3.15 (m, 1H), 3.08 (dd, J=14.2, 8.6 Hz, 1H), 2.35-2.28 (m, 1H), 2.24-2.20 (m, 3H), 2.18-1.96 (m, 3H), 1.71 (dd, J=13.0, 8.9 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 10% B to 40% B in 10 min; Wave Length: 254 nm/220 nm; RT1(min): 9.3.

### Synthetic Procedure of (2R,3S,4S)-4-hydroxy-2-{[4′-(pentafluoro-lambda6-sulfanyl)-[1,1′-biphenyl]-4-yl]methyl}pyrrolidin-3-yl N-(1,2,3,4-tetrahydroisoquinolin-6-ylmethyl)carbamate (V192059)

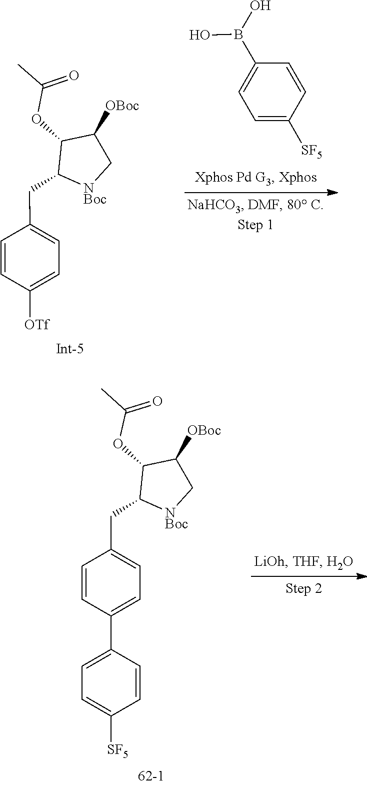

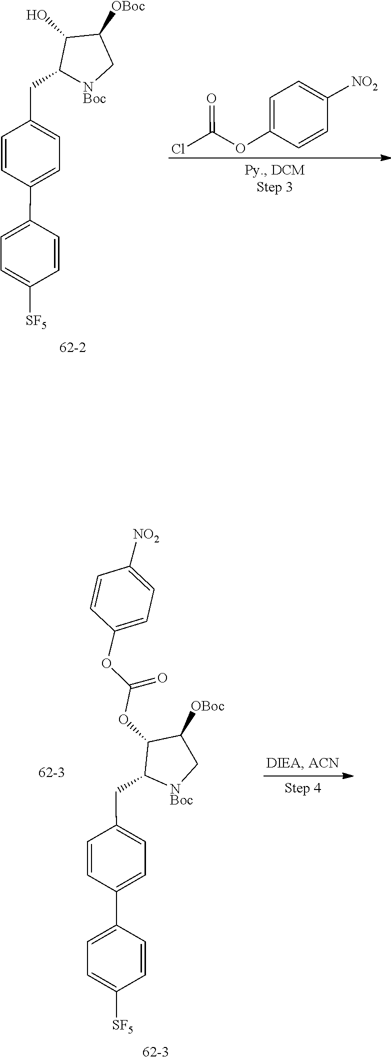

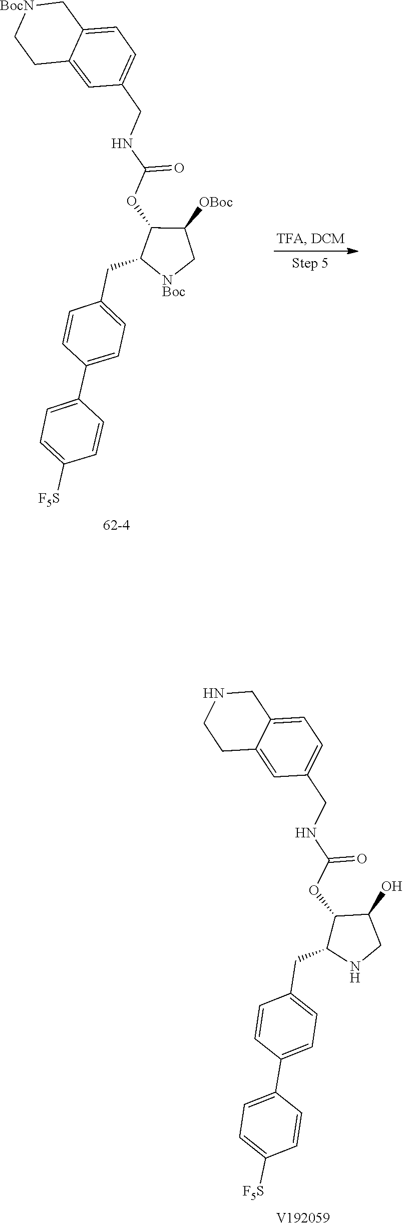

Step 1: To a stirred mixture of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (290 mg, 0.47 mmol, 1 equiv) and 4-(pentafluoro-lambda6-sulfanyl)phenylboronic acid (184.8 mg, 0.74 mmol, 1.5 equiv) in DMF (5 mL) was added XPhos (47.38 mg, 0.099 mmol, 0.2 equiv) XPhos Pd G₃ (23.6 mg, 0.05 mmol, 0.1 equiv) and NaHCO₃ (83.9 mg, 0.94 mmol, 2 equiv) at room temperature under nitrogen atmosphere. The resulting mixture was stirred at 80° C. for overnight under nitrogen atmosphere. The resulting mixture was filtered, the filter cake was washed with DMF (1×2 mL). The residue was purified by reversed-phase flash chromatography to afford tert-butyl (2R,3S,4S)-3-(acetyloxy)-4-[(tert-butoxycarbonyl)oxy]-2-{[4′-(pentafluoro-lambda6-sulfanyl)-[1,1′-biphenyl]-4-yl]methyl}pyrrolidine-1-carboxylate (302 mg) as a yellow solid. MS: m/z: Calc'd for C₂₉H₃₆F₅NO₇S [M+22]⁺660. found 660.

Step 2: To a stirred mixture of tert-butyl (2R,3S,4S)-3-(acetyloxy)-4-[(tert-butoxycarbonyl)oxy]-2-{[4′-(pentafluoro-lambda6-sulfanyl)-[1,1′-biphenyl]-4-yl]methyl}pyrrolidine-1-carboxylate (302 mg, 0.47 mmol, 1 equiv) in THF (5 mL) and H₂O (0.5 mL) was added LiOH (34.3 mg, 1.42 mmol, 3 equiv) at room temperature. The resulting mixture was stirred at room temperature for overnight. The mixture/residue was acidified/basified/neutralized to pH 7 with saturated NaHCO₃. The reaction mixture was quenched with water and extracted with EA. The extracts were washed with brine, dried over anhydrous sodium sulfate, filtered and concentrated to afford crude tert-butyl (2R,3S,4S)-4-[(tert-butoxycarbonyl)oxy]-3-hydroxy-2-{[4′-(pentafluoro-lambda6-sulfanyl)-[1,1′-biphenyl]-4-yl]methyl}pyrrolidine-1-carboxylate (277 mg, 98.2%) as a white solid which was used directly in the next step without further purification.

Step 3: To a stirred mixture of tert-butyl (2R,3S,4S)-4-[(tert-butoxycarbonyl)oxy]-3-hydroxy-2-{[4′-(pentafluoro-lambda6-sulfanyl)-[1,1′-biphenyl]-4-yl]methyl}pyrrolidine-1-carboxylate (277 mg, 0.46 mmol, 1 equiv) and 4-nitrophenyl carbonochloridate (281.1 mg, 1.39 mmol, 3 equiv) in DCM (5 mL) was added Pyridine (147.1 mg, 1.86 mmol, 4 equiv) at room temperature. The resulting mixture was stirred at room temperature for overnight. The resulting mixture was filtered, the filter cake was washed with DCM (1×2 mL). The filtrate was concentrated under reduced pressure. The residue was purified by Prep-TLC (PE/EA 3:1) to afford tert-butyl (2R,3S,4S)-4-[(tert-butoxycarbonyl)oxy]-3-[(4-nitrophenoxycarbonyl)oxy]-2-{[4′-(pentafluoro-lambda6-sulfanyl)-[1,1′-biphenyl]-4-yl]methyl}pyrrolidine-1-carboxylate (150 mg, 42.4%) as a white solid. MS: m/z: Calc'd for C₃₄H₃₇F₅N₂O₁₀S [M−100]⁺661. found 661.

Step 4 and 5: The title compound was prepared in 41.3% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4′-(pentafluoro-lambda6-sulfanyl)-[1,1′-biphenyl]-4-yl]methyl}pyrrolidine-1-carboxylate (62-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 6-(aminomethyl)-3,4-dihydro-1H-isoquinoline-2-carboxylate in STEP 4; Boc Deprotection; General Procedure II in STEP 5. MS: m/z C₂₈H₃₀F₅N₃O₃S [M+H]⁺584. found 584. ¹H NMR (400 MHz, Methanol-d₄) δ 7.95-7.88 (m, 2H), 7.81 (d, J=8.5 Hz, 2H), 7.72-7.66 (m, 2H), 7.46 (d, J=8.0 Hz, 2H), 7.35-7.17 (m, 3H), 5.01 (d, J=4.7 Hz, 1H), 4.47-4.19 (m, 6H), 3.63 (dd, J=12.6, 4.3 Hz, 1H), 3.49-3.39 (m, 2H), 3.25 (dd, J=13.4, 6.8 Hz, 2H), 3.13-3.03 (m, 3H).

Prep-HPLC purification conditions: Column: Xselect CSH Prep C18 OBD Column, 30*150 mm, 5 μm; Mobile Phase A: Water (0.05% TFA), Mobile Phase B: ACN; Flow rate: 60 mL/min; Gradient: 11% B to 41% B in 7 min; Wave Length: 254 nm/220 nm; RT1(min): 6.78.

### Synthetic Procedure of (2R,3S,4S)-4-hydroxy-2-{[4′-(pentafluoro-lambda6-sulfanyl)-[1,1′-biphenyl]-4-yl]methyl}pyrrolidin-3-yl N-{2-[(2R)-pyrrolidin-2-yl]ethyl}carbamate (V192039)

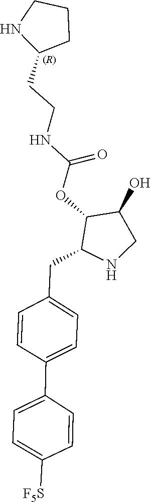

The title compound was prepared in 37.9% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4′-(pentafluoro-lambda6-sulfanyl)-[1,1′-biphenyl]-4-yl]methyl}pyrrolidine-1-carboxylate (62-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2R)-2-(2-aminoethyl)pyrrolidine-1-carboxylate in STEP 1; Boc Deprotection; General Procedure II in STEP 2. MS: m/z Calc'd for C₂₄H₃₀F₅N₃O₃S [M+H]⁺536. found 536. ¹H NMR (400 MHz, Methanol-d₄) δ 7.95-7.88 (m, 2H), 7.82 (d, J=8.6 Hz, 2H), 7.76-7.70 (m, 2H), 7.48 (d, J=8.0 Hz, 2H), 5.04 (d, J=3.6 Hz, 1H), 4.42 (d, J=3.9 Hz, 1H), 4.32 (d, J=3.4 Hz, 1H), 3.58 (dd, J=15.0, 13.5 Hz, 2H), 3.33-3.26 (m, 4H), 3.24 (d, J=5.4 Hz, 1H), 3.21-3.16 (m, 1H), 3.14-3.06 (m, 1H), 2.35-2.26 (m, 1H), 2.01 (ddd, J=48.2, 21.0, 13.9 Hz, 4H), 1.71 (dd, J=12.5, 8.8 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min mL/min; Gradient: 16% B to 46% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-[(2,6-difluorophenyl)methyl]carbamate (V191992)

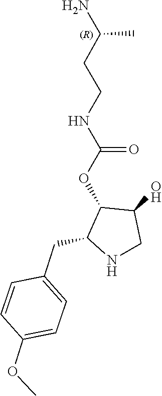

The title compound was prepared in 32.0% overall yield as a white solid according to Substitution reaction for synthesis of Carbamate; General Procedure I using tert-butyl N-[(2R)-4-aminobutan-2-yl]carbamate in STEP 1; Boc Deprotection; General Procedure II in STEP 2. MS: m/z Calc'd for C₁₇H₂₇N₃O₄ [M+H]⁺338. Found, 338. ¹H NMR (400 MHz, Methanol-d₄) δ 7.28-7.20 (m, 2H), 6.96-6.88 (m, 2H), 4.96 (d, J=3.6 Hz, 1H), 4.39 (d, J=4.2 Hz, 1H), 4.19-4.14 (m, 1H), 3.80 (s, 3H), 3.59 (dd, J=12.7, 4.4 Hz, 1H), 3.43-3.36 (m, 1H), 3.31-3.28 (m, 2H), 3.22 (d, J=12.6 Hz, 1H), 3.11 (dd, J=14.1, 7.2 Hz, 1H), 2.99 (dd, J=14.0, 8.6 Hz, 1H), 2.00-1.88 (m, 1H), 1.83-1.74 (m, 1H), 1.36 (d, J=6.6 Hz, 3H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 25% B in 15 min; Wave Length: 254 nm/220 nm; RT1(min): 16.5.

### Synthetic Procedure of (2R,3S,4S)-4-hydroxy-2-[(4-methoxyphenyl)methyl]pyrrolidin-3-yl N-{2-[(2R)-1-methylpyrrolidin-2-yl]ethyl}carbamate (V192042)

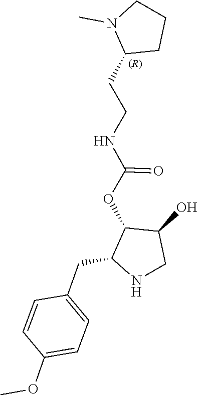

The title compound was prepared in 44.1% overall yield as a white solid according to Substitution reaction for synthesis of Carbamate; General Procedure I using tert-butyl N-[(2R)-4-aminobutan-2-yl]carbamate in STEP 1; Boc Deprotection; General Procedure II in STEP 2. MS: m/z Calc'd for C₂₀H₃₁N₃O₄ [M+H]⁺378. Found, 378. ¹H NMR (400 MHz, Methanol-d₄) δ 7.29-7.21 (m, 2H), 6.96-6.89 (m, 2H), 4.97 (d, J=3.6 Hz, 1H), 4.39 (d, J=4.2 Hz, 1H), 4.22-4.12 (m, 1H), 3.80 (s, 3H), 3.74-3.67 (m, 1H), 3.59 (dd, J=12.9, 4.2 Hz, 1H), 3.35 (s, 1H), 3.30-3.27 (m, 2H), 3.24 (d, J=8.3 Hz, 1H), 3.21-3.07 (m, 2H), 3.03-2.77 (m, 4H), 2.48-2.41 (m, 1H), 2.27-2.03 (m, 3H), 1.87-1.77 (m, 2H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 30% B in 15 min; Wave Length: 254 nm/220 nm; RT1(min): 16.5.

### Synthetic Procedure of (2R,3S,4S)-4-hydroxy-2-{[4-(1,3-oxazol-5-yl)phenyl]methyl}pyrrolidin-3-yl N-(1,2,3,4-tetrahydroisoquinolin-6-ylmethyl)carbamate (V192056)

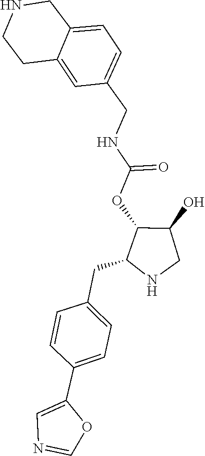 [SMILES: O=C(NCc1ccc2c(c1)CCNC2)O[C@@H]1[C@@H](O)CN[C@@H]1Cc1ccc(-c2cnco2)cc1]

The title compound was prepared in 8.4% overall yield as a light yellow according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 6-(aminomethyl)-3,4-dihydro-1H-isoquinoline-2-carboxylate in STEP 1; Boc Deprotection; General Procedure II in STEP 2. MS: m/z Calc'd for C₂₅H₂₈N₄O₄ [M+H]⁺449. Found, 449. ¹H NMR (400 MHz, Methanol-d₄) δ 8.28 (s, 1H), 7.73 (d, J=7.9 Hz, 2H), 7.54 (s, 1H), 7.43 (d, J=7.9 Hz, 2H), 7.29-7.23 (m, 3H), 5.00 (d, J=3.5 Hz, 1H), 4.43-4.25 (m, 6H), 3.62 (dd, J=12.7, 4.3 Hz, 1H), 3.53-3.51 (m, 2H), 3.28-3.19 (m, 2H), 3.15-3.04 (m, 3H).

Prep-HPLC purification conditions: Column: Xbridge Prep OBD Shield RP18, 19*250 mm, 5 μm; Mobile Phase A: Water (0.05% TFA), Mobile Phase B: MEOH; Flow rate: 25 mL/min; Gradient: 12% B to 39% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-[2-(2,3-dihydro-1H-isoindol-5-yl)ethyl]carbamate (V188344)

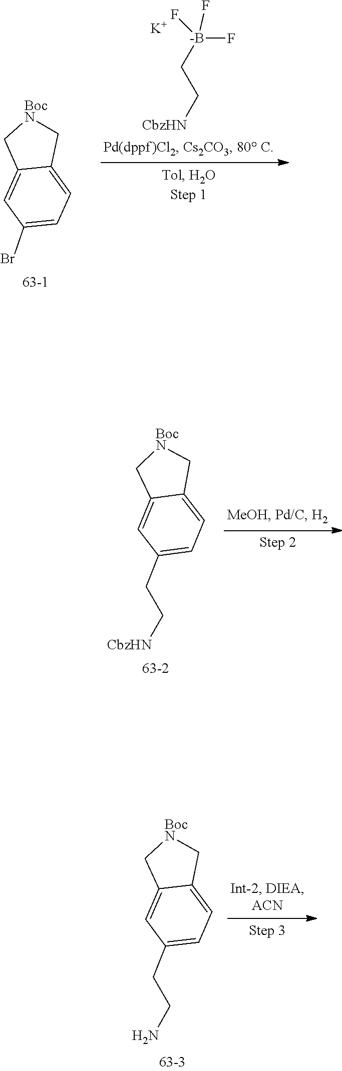

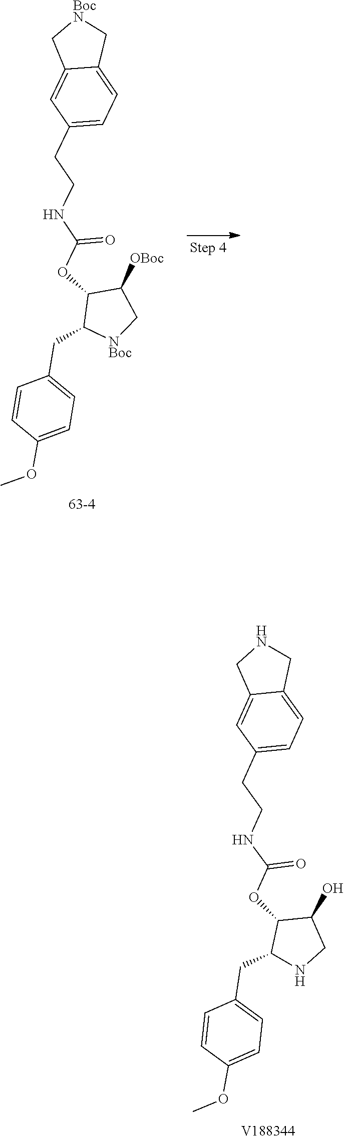

Step 1: To a stirred mixture of benzyl N-[2-(trifluoroboranuidyl)ethyl]carbamate (495.4 mg, 2.01 mmol, 1.2 equiv) and tert-butyl 5-bromo-1,3-dihydroisoindole-2-carboxylate (500 mg, 1.67 mmol, 1.0 equiv) in toluene (8 mL) and Water (2 mL) was added Pd(dppf)Cl₂CH₂Cl₂ (122.7 mg, 0.16 mmol, 0.1 equiv) and Cs₂CO₃ (1639.0 mg, 5.01 mmol, 3equiv) at room temperature under nitrogen atmosphere. The resulting mixture was stirred at 80° C. for overnight under nitrogen atmosphere. The resulting mixture was concentrated under reduced pressure. The residue was dissolved in MeCN (5 mL). The residue was purified by reversed-phase flash chromatography to afford tert-butyl 5-(2-{[(benzyloxy)carbonyl]amino}ethyl)-1,3-dihydroisoindole-2-carboxylate (500 mg, 90% purity) as a white solid. MS: m/z: Calc'd for C₁₀H1₂BF₃NO₂ [M−100]⁺297. found 297.

Step 2: To a stirred mixture of tert-butyl 5-(2-{[(benzyloxy)carbonyl]amino}ethyl)-1,3-dihydroisoindole-2-carboxylate (200 mg, 0.54 mmol, 1 equiv) in EA (5 mL) was added Pd/C (134.2 mg, 1.20 mmol, 2.5 equiv) at room temperature under nitrogen atmosphere. The resulting mixture was stirred for overnight at room temperature under hydrogen atmosphere. The resulting mixture was filtered, the filter cake was washed with EA (2×2 mL). The filtrate was concentrated under reduced pressure. This resulted in tert-butyl 5-(2-aminoethyl)-1,3-dihydroisoindole-2-carboxylate (100 mg, 75.5%) as a yellow oil. MS: m/z: Calc'd for C₁₅H₂₂N₂O₂ [M+H]⁺263. found 263.

Step 3 and 4: The title compound was prepared in 23.7% overall yield as a yellow solid according to Substitution reaction for synthesis of Carbamate; General Procedure I using tert-butyl 5-(2-aminoethyl)-1,3-dihydroisoindole-2-carboxylate (63-3) in STEP 3; Boc Deprotection; General Procedure II in STEP 4. MS: m/z Calc'd for C₂₃H₂₉N₃O₄ [M+H]⁺412. found 412. ¹H NMR (400 MHz, Methanol-d₄) δ 7.43-7.27 (m, 3H), 7.23-7.11 (m, 2H), 7.06-6.86 (m, 2H), 4.92 (d, J=3.5 Hz, 1H), 4.58 (d, J=16.1 Hz, 4H), 4.33 (d, J=4.3 Hz, 1H), 4.11-4.01 (m, 1H), 3.79 (s, 3H), 3.56 (dd, J=12.7, 4.4 Hz, 1H), 3.49-3.33 (m, 2H), 3.20 (d, J=12.6 Hz, 1H), 3.04 (dd, J=14.1, 7.1 Hz, 1H), 2.97-2.82 (m, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 29% B in 10 min; Wave Length: 254 nm/220 nm; RT1(min): 8.5.

### Synthetic Procedure of (2R,3S,4S)-2-{[4-(2,3-dihydro-1H-isoindol-5-yl)phenyl]methyl}-4-hydroxypyrrolidin-3-yl N-benzylcarbamate (V188347)

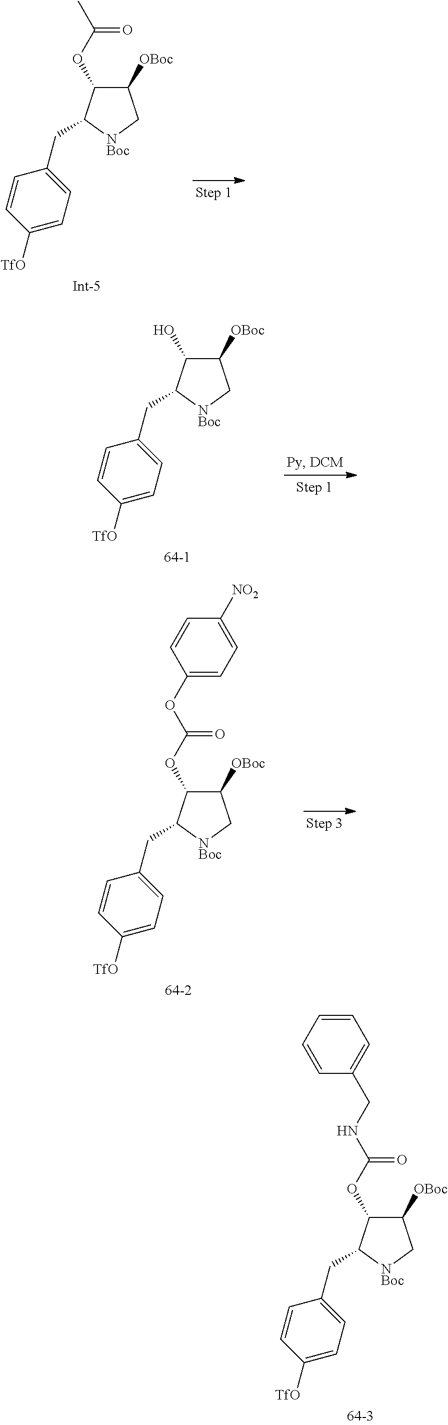

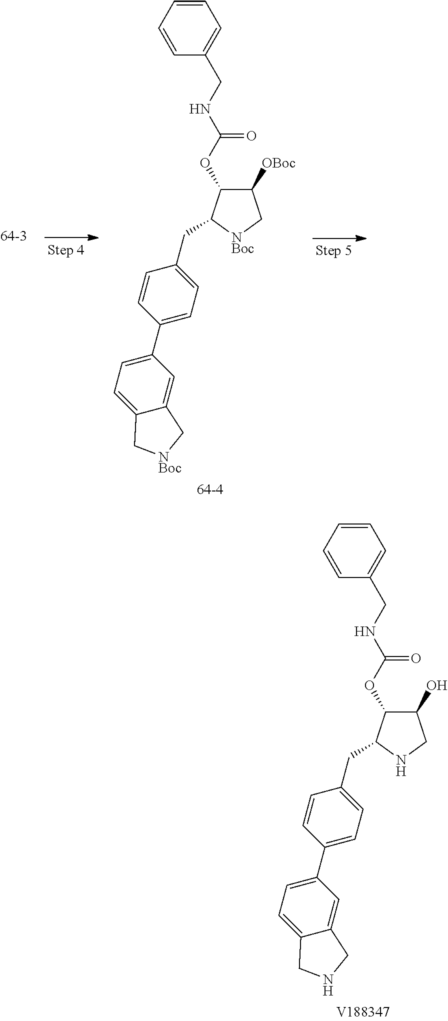

Step 1: To a stirred mixture of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (100 mg, 0.17 mmol, 1 equiv) in H₂O (1 mL, 0.85 mmol) and THF (20 mL) was added LiOH (4.1 mg, 0.17 mmol, 1 equiv) at room temperature. The resulting mixture was stirred for overnight at room temperature. The residue was purified by reversed-phase flash chromatography to afford tert-butyl (2R,3S,4S)-4-[(tert-butoxycarbonyl)oxy]-3-hydroxy-2-{[4-(trifluoromethanesulfonyloxy)phenyl]methyl}pyrrolidine-1-carboxylate (80 mg, 86.2%) as a yellow oil. MS: m/z: Calc'd for C₂₂H₃₀F₃NO₉S [M+22]⁺564. found 564.

Step 2: To a stirred mixture of tert-butyl (2R,3S,4S)-4-[(tert-butoxycarbonyl)oxy]-3-hydroxy-2-{[4-(trifluoromethanesulfonyloxy)phenyl]methyl}pyrrolidine-1-carboxylate (85 mg, 0.15 mmol, 1 equiv) and 4-nitrophenyl chloroformate (47.4 mg, 0.23 mmol, 1.5 equiv) in DCM (5 mL) was added Pyridine (37.2 mg, 0.47 mmol, 3 equiv) at room temperature. The resulting mixture was stirred for overnight at room temperature. The resulting mixture was filtered, the filter cake was washed with DCM (1×2 mL). The filtrate was concentrated under reduced pressure. The residue was dissolved in DCM (2 mL). The residue was purified by Prep-TLC (PE/EA 3:1) to afford tert-butyl (2R,3S,4S)-4-[(tert-butoxycarbonyl)oxy]-3-[(4-nitrophenoxycarbonyl)oxy]-2-{[4-(trifluoromethanesulfonyloxy)phenyl]methyl}pyrrolidine-1-carboxylate (50 mg, 45.1%) as a white solid. MS: m/z: Calc'd for C₂₉H₃₃F₃N₂O₁₃S [M+22]+ 729. found 729.

Step 3: The title compound was prepared in 83.7% yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(trifluoromethanesulfonyloxy)phenyl]methyl}pyrrolidine-1-carboxylate (64-2) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), benzylamine. MS: m/z C₃₀H₃₇F₃N₂O₁₀S [M+22]⁺697. found 697.

Step 4: To a stirred mixture of tert-butyl 5-bromo-1,3-dihydroisoindole-2-carboxylate (19.9 mg, 0.067 mmol, 1.1 equiv) and tert-butyl (2R,3S,4S)-3-[(benzylcarbamoyl)oxy]-4-[(tert-butoxycarbonyl)oxy]-2-{[4-(trifluoromethanesulfonyloxy)phenyl]methyl}pyrrolidine-1-carboxylate (64-3, 40 mg, 0.06 mmol, 1.00 equiv) NaHCO₃ (10.2 mg, 0.12 mmol, 2 equiv) in DMF (5 mL) was added XPhos (5.7 mg, 0.01 mmol, 0.2 equiv) and XPhosPdG₄ (5.2 mg, 0.006 mmol, 0.1 equiv) at room temperature under nitrogen atmosphere. The resulting mixture was stirred at 80° C. for overnight under nitrogen atmosphere. The residue was purified by reversed-phase flash chromatography to afford tert-butyl 5-(4-{[(2R,3S,4S)-3-[(benzylcarbamoyl)oxy]-1-(tert-butoxycarbonyl)-4-[(tert-butoxycarbonyl)oxy]pyrrolidin-2-yl]methyl}phenyl)-1,3-dihydroisoindole-2-carboxylate (45 mg, 90% purity) as a yellow solid. MS: m/z: Calc'd for C₄₂H₅₃N₃O₉ [M+H]⁺744. found 744.

Step 5: The title compound was prepared in 32.7% yield as a white solid according to Boc Deprotection; General Procedure II. MS: m/z C₂₇H₂₉N₃O₃ [M+H]⁺444. found 444. ¹H NMR (400 MHz, Methanol-d₄) δ 7.71-7.63 (m, 2H), 7.62-7.57 (m, 2H), 7.52 (d, J=7.9 Hz, 1H), 7.44-7.18 (m, 7H), 4.98-4.93 (m, 1H), 4.69 (d, J=8.5 Hz, 4H), 4.55-4.31 (m, 3H), 4.31-4.15 (m, 1H), 3.61 (dd, J=12.6, 4.3 Hz, 1H), 3.26 (d, J=12.4 Hz, 1H), 3.23-3.06 (m, 2H).

Prep-HPLC purification conditions: Column: Xselect CSH Prep C18 OBD Column, 30*150 mm, 5 μm; Mobile Phase A: Water (0.05% TFA), Mobile Phase B: ACN; Flow rate: 60 mL/min; Gradient: 11% B to 41% B in 7 min; Wave Length: 254 nm/220 nm; RT1(min): 6.78.

### Synthetic Procedure of (2R,3S,4S)-4-hydroxy-2-[(4-methoxyphenyl)methyl]pyrrolidin-3-yl N-(3-methylbut-2-en-1-yl)carbamate (V190411)

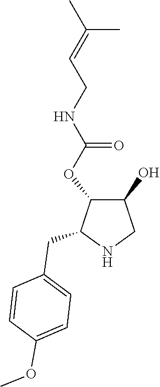 [SMILES: COc1ccc(C[C@H]2NC[C@H](O)[C@H]2OC(=O)NCC=C(C)C)cc1]

The title compound was prepared in 25.7% overall yield as a yellow semi-solid according to Substitution reaction for synthesis of Carbamate; General Procedure I using prenylamine in STEP 1; Boc Deprotection; General Procedure II in STEP 2. MS: m/z Calc'd for C₁₈H₂₆N₂O₄ [M+H]⁺335. found 335. ¹H NMR (400 MHz, Methanol-d₄) δ 7.26-7.18 (m, 2H), 6.96-6.88 (m, 2H), 5.29-5.20 (m, 1H), 4.92 (d, J=3.5 Hz, 1H), 4.37 (d, J=4.2 Hz, 1H), 4.14-4.02 (m, 1H), 3.79 (s, 5H), 3.57 (dd, J=12.6, 4.4 Hz, 1H), 3.20 (d, J=12.7 Hz, 1H), 3.03-2.91 (m, 2H), 1.79-1.71 (m, 6H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 18% B to 28% B in 15 min; Wave Length: 254 nm/220 nm; RT1(min): 16.5.

### Synthetic Procedure of (2R,3S,4S)-4-hydroxy-2-[(4-methoxyphenyl)methyl]pyrrolidin-3-yl N-(3-hydroxy-3-methylbutyl)carbamate (V190413)

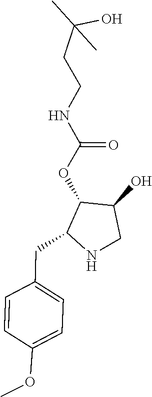 [SMILES: COc1ccc(C[C@H]2NC[C@H](O)[C@H]2OC(=O)NCCC(C)(C)O)cc1]

The title compound was prepared in 51.0% overall yield as a white solid according to Substitution reaction for synthesis of Carbamate; General Procedure I using 4-amino-2-methylbutan-2-ol in STEP 1; Boc Deprotection; General Procedure II in STEP 2. MS: m/z Calc'd for C₁₈H₂₈N₂O₅ [M+H]⁺353. found 353. ¹H NMR (400 MHz, Methanol-d₄) δ 7.27-7.18 (m, 2H), 6.96-6.87 (m, 2H), 4.99 (s, 1H), 4.44-4.34 (m, 1H), 4.14-4.01 (m, 1H), 3.79 (s, 3H), 3.58 (dd, J=12.7, 4.5 Hz, 1H), 3.40-3.17 (m, 3H), 3.16-2.93 (m, 2H), 1.78-1.63 (m, 2H), 1.25 (s, 6H).

Prep-HPLC purification conditions: Column: Welch Ultimate XB-Phenyl 30*250, 10 μm; Mobile Phase A: Water (0.05% TFA), Mobile Phase B: ACN; Flow rate: 60 mL/min; Gradient: 6% B to 36% B in 10 min; Wave Length: 254 nm/220 nm; RT1(min): 8.5.

### Synthetic Procedure of (2R,3S,4S)-2-[(4-cyclopropoxyphenyl)methyl]-4-hydroxypyrrolidin-3-ylN-[(3-fluorophenyl)methyl]carbamate (V190516)

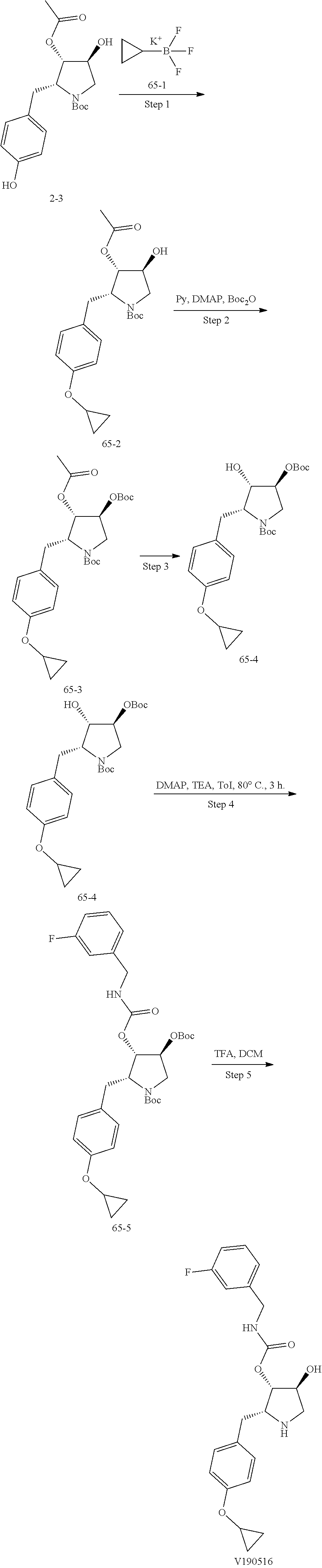

Step 1: To a stirred mixture of tert-butyl (2R,3S,4S)-3-(acetyloxy)-4-hydroxy-2-[(4-hydroxyphenyl)methyl]pyrrolidine-1-carboxylate (2-3, 300 mg, 0.85 mmol, 1 equiv) and potassium cyclopropyltrifluoroboranuide (378.9 mg, 2.56 mmol, 3 equiv) in Toluene (10 mL) and H₂O (1 mL) was added 1,10-phenanthroline (15.38 mg, 0.085 mmol, 0.1 equiv), K₂CO₃ (353.97 mg, 2.56 mmol, 3 equiv) and Cu(OAc)₂ (38.7 mg, 0.21 mmol, 0.25 equiv) at room temperature. The resulting mixture was stirred for overnight at 70° C. under nitrogen atmosphere. The resulting mixture was filtered, the filter cake was washed with Toluene (1×2 mL). The filtrate was concentrated under reduced pressure. The residue was dissolved in MeCN (5 mL). The residue was purified by reversed-phase flash chromatography to afford tert-butyl (2R,3S,4S)-3-(acetyloxy)-2-[(4-cyclopropoxyphenyl)methyl]-4-hydroxypyrrolidine-1-carboxylate (190 mg, 56.8%) as a yellow solid. MS: m/z: Calc'd for C₂₁H₂₉NO₆ [M+22]⁺414. found 414.

Step 2: To a stirred mixture of tert-butyl (2R,3S,4S)-3-(acetyloxy)-2-[(4-cyclopropoxyphenyl)methyl]-4-hydroxypyrrolidine-1-carboxylate (190 mg, 0.45 mmol, 1 equiv) and Boc₂O (566.0 mg, 2.42 mmol, 5 equiv) in Pyridine (5 mL) was added DMAP (88.9 mg, 0.72 mmol, 1.5 equiv) at room temperature. The resulting mixture was stirred for overnight at room temperature. The resulting mixture was concentrated under vacuum. The residue was dissolved in MeCN (5 mL). The residue was purified by reversed-phase flash chromatography to afford tert-butyl (2R,3S,4S)-3-(acetyloxy)-4-[(tert-butoxycarbonyl)oxy]-2-[(4-cyclopropoxyphenyl)methyl]pyrrolidine-1-carboxylate (90 mg, 37.2%) as a yellow oil. MS: m/z: Calc'd for C₂₆H₃₇NO₈ [M+22]⁺514. found 514.

### Step 3, 4, and 5

The title compound was prepared in 36.3% overall yield as a white solid according to Hydrolysis reaction with LiOH; General Procedure V using tert-butyl (2R,3S,4S)-3-(acetyloxy)-4-[(tert-butoxycarbonyl)oxy]-2-[(4-cyclopropoxyphenyl)methyl]pyrrolidine-1-carboxylate (65-3) in STEP 3; Addition reaction: Isocyanates and hydroxyl groups; General Procedure VI using tert-butyl (2R,3S,4S)-4-[(tert-butoxycarbonyl)oxy]-2-[(4-cyclopropoxyphenyl)methyl]-3-hydroxypyrrolidine-1-carboxylate (65-4) instead of tert-butyl (2R,3S,4S)-4-[(tert-butoxycarbonyl)oxy]-2-[(4-fluorophenyl)methyl]-3-hydroxypyrrolidine-1-carboxylate (Int-7), 1-fluoro-3-(isocyanatomethyl)benzene in STEP 4; Boc Deprotection; General Procedure II in STEP 5. MS: m/z Calc'd for C₂₂H₂₅FN₂O₄[M+H]⁺401. found 401. ¹H NMR (400 MHz, Methanol-d₄) δ 7.38-7.28 (m, 1H), 7.24-7.18 (m, 2H), 7.18-7.07 (m, 2H), 7.06-6.97 (m, 3H), 4.96-4.91 (m, 1H), 4.41 (d, J=4.8 Hz, 1H), 4.40-4.28 (m, 2H), 4.16-4.06 (m, 1H), 3.76-3.66 (m, 1H), 3.59 (dd, J=12.4, 4.6 Hz, 1H), 3.23 (d, J=12.6 Hz, 1H), 3.11 (dd, J=12.8, 6.6 Hz, 1H), 2.98 (dd, J=13.9, 7.9 Hz, 1H), 0.84-0.71 (m, 2H), 0.68-0.58 (m, 2H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 9.08.

### Synthetic Procedure of (2R,3S,4S)-2-{[4-(1,1-difluoroethyl)phenyl]methyl}-4-hydroxypyrrolidin-3-yl N-[(3-fluorophenyl)methyl]carbamate (V190547)

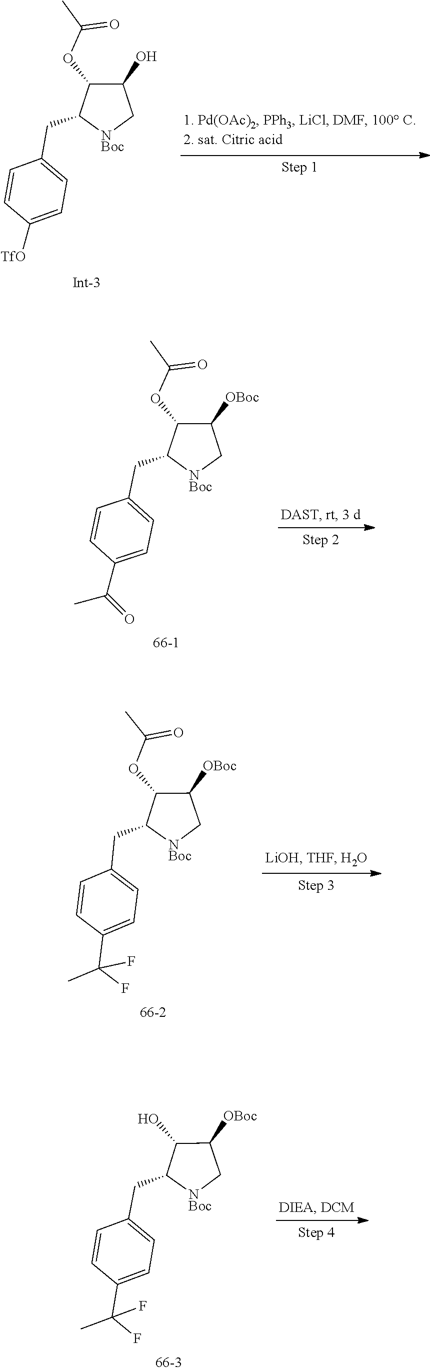

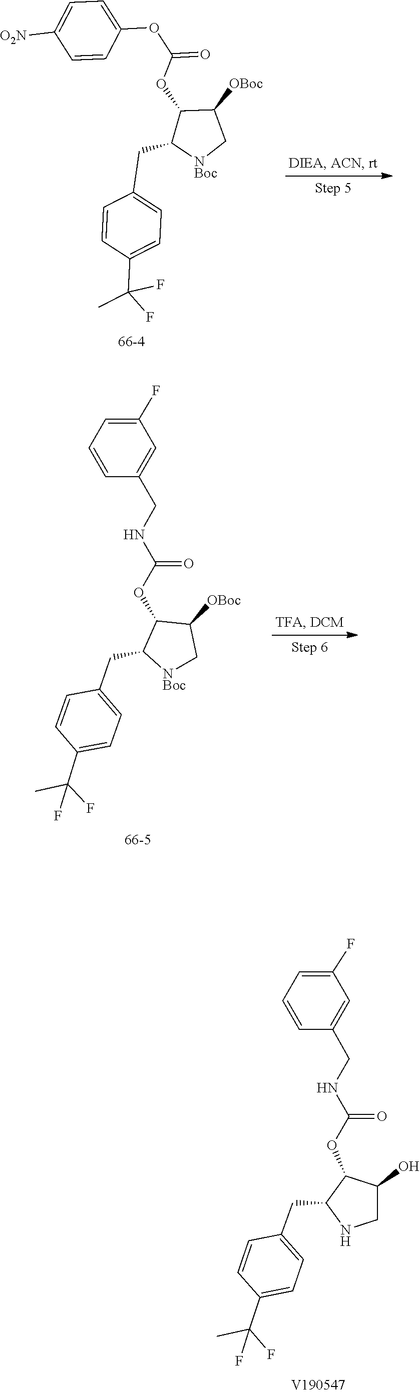

Step 1: To the solution of tert-butyl (2R,3S,4S)-3-(acetyloxy)-4-hydroxy-2-{[4-(trifluoromethanesulfonyloxy)phenyl]methyl}pyrrolidine-1-carboxylate (380 mg, 0.79 mmol, 1 equiv), tributyl(1-ethoxyethenyl)stannane (426 mg, 1.18 mmol, 1.5 equiv) and LiCl (67 mg, 1.57 mmol, 2 equiv) in DMF (5 mL) was added Pd(OAc)₂ (18 mg, 0.079 mmol, 0.1 equiv) and PPh₃ (41 mg, 0.16 mmol, 0.2 equiv). The mixture was stirred at 100° C. for 4 hours under N₂. Citric acid (sat., aq., 5 mL) was added and stirred for 1 hour at rt. The mixture was diluted with EA, washed with water, dried and concentrated. The residue was purified by Prep-TLC (PE/EA 2:1) to afford tert-butyl (2R,3S,4S)-3-(acetyloxy)-2-[(4-acetylphenyl)methyl]-4-[(tert-butoxycarbonyl)oxy]pyrrolidine-1-carboxylate (200 mg, 53.3%) as a light yellow oil. MS: m/z: Calc'd for C₂₅H₃₅NO₈ [M+H]⁺478. found 478.

Step 2: The solution of tert-butyl (2R,3S,4S)-3-(acetyloxy)-2-[(4-acetylphenyl)methyl]-4-[(tert-butoxycarbonyl)oxy]pyrrolidine-1-carboxylate (200 mg, 0.42 mmol, 1 equiv) in DAST (6 mL, 0.21 mmol) was stirred at rt for 3 days. The mixture was concentrated to give the crude product which was purified by reversed phase flash column to afford tert-butyl (2R,3S,4S)-3-(acetyloxy)-4-[(tert-butoxycarbonyl)oxy]-2-{[4-(1,1-difluoroethyl)phenyl]methyl}pyrrolidine-1-carboxylate (40 mg, 19.1%) as a brown oil. MS: m/z: Calc'd for C₂₅H₃₅F₂NO₇ [M+Na]⁺522. found 522.

Step 3: The title compound was prepared in 97.1% yield as a brown oil according to Hydrolysis reaction with LiOH; General Procedure V using tert-butyl (2R,3S,4S)-3-(acetyloxy)-4-[(tert-butoxycarbonyl)oxy]-2-{[4-(1,1-difluoroethyl)phenyl]methyl}pyrrolidine-1-carboxylate (66-2). MS: m/z: Calc'd for C₂₃H₃₃F₂NO₆ [M+Na]⁺480. found 480.

Step 4: To the solution of tert-butyl (2R,3S,4S)-4-[(tert-butoxycarbonyl)oxy]-2-{[4-(1,1-difluoroethyl)phenyl]methyl}-3-hydroxypyrrolidine-1-carboxylate (66-3, 40 mg, 0.087 mmol, 1 equiv) and 4-nitrophenyl carbonochloridate (26 mg, 0.13 mmol, 1.5 equiv) in DCM (2 mL) was added Pyridine (14 mg, 0.17 mmol, 2 equiv). The mixture was stirred at rt for 2 hours. The mixture was concentrated to give the crude product which was purified by reversed phase flash column to afford tert-butyl (2R,3S,4S)-4-[(tert-butoxycarbonyl)oxy]-2-{[4-(1,1-difluoroethyl)phenyl]methyl}-3-[(4-nitrophenoxycarbonyl)oxy]pyrrolidine-1-carboxylate (28 mg, 51.4%) as a colorless oil. MS: m/z: Calc'd for C₃₀H₃₆F₂N₂O₁₀ [M+Na]⁺645. found 645.

### Step 5 and 6

The title compound was prepared in 33.1% overall yield as a white solid according to Substitution reaction for synthesis of Carbamate; General Procedure I using tert-butyl (2R,3S,4S)-4-[(tert-butoxycarbonyl)oxy]-2-{[4-(1,1-difluoroethyl)phenyl]methyl}-3-[(4-nitrophenoxycarbonyl)oxy]pyrrolidine-1-carboxylate (66-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 5; Boc Deprotection; General Procedure II in STEP 6. MS: m/z Calc'd for C₂₁H₂₃F₃N₂O3[M+H]⁺409. found 409. ¹H NMR (400 MHz, Methanol-d₄) δ 7.45-7.27 (m, 6H), 7.18-6.97 (m, 4H), 4.69-4.60 (m, 1H), 4.44-4.24 (m, 2H), 4.21-4.14 (m, 1H), 3.64-3.55 (m, 1H), 3.40-3.29 (m, 1H), 2.98-2.83 (m, 2H), 2.79-2.62 (m, 1H), 1.89 (t, J=18.2 Hz, 3H).

Prep-HPLC purification conditions: Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 55% B in 10 min; Wave Length: 254 nm/220 nm; RT1(min): 8.68.

### Synthetic Procedure of (2R,3S,4S)-2-{[4-(1,1-difluoroethyl)phenyl]methyl}-4-hydroxypyrrolidin-3-yl N-{2-[(2S)-pyrrolidin-2-yl]ethyl}carbamate (V191885)

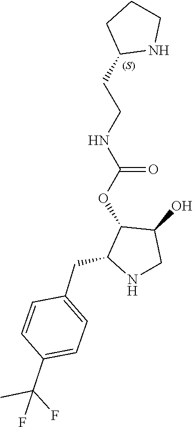

The title compound was prepared in 40.3% overall yield as an off-white solid according to Substitution reaction for synthesis of Carbamate; General Procedure I using tert-butyl (2R,3S,4S)-4-[(tert-butoxycarbonyl)oxy]-2-{[4-(1,1-difluoroethyl)phenyl]methyl}-3-[(4-nitrophenoxycarbonyl)oxy]pyrrolidine-1-carboxylate (66-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2R)-2-(2-aminoethyl)pyrrolidine-1-carboxylate in STEP 1; Boc Deprotection; General Procedure II in STEP 2. MS: m/z Calc'd for C₂₀H₂₉F₂N₃O₃[M+H]⁺398, found 398. ¹H NMR (400 MHz, Methanol-d₄) δ 7.55 (d, J=7.9 Hz, 2H), 7.44 (d, J=8.0 Hz, 2H), 5.01-4.95 (m, 1H), 4.40 (d, J=4.1 Hz, 1H), 4.29-4.20 (m, 1H), 3.68-3.50 (m, 1H), 3.32-3.18 (m, 6H), 3.16-3.06 (m, 1H), 2.37-2.24 (m, 1H), 2.18-1.83 (m, 7H), 1.79-1.64 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 26% B in 10 min; Wave Length: 254 nm/220 nm; RT1(min): 9.88.

### Synthetic Procedure of (2R,3S,4S)-4-hydroxy-2-[(4-methoxyphenyl)methyl]pyrrolidin-3-yl N-{2-[(2S)-azetidin-2-yl]ethyl}carbamate (V191920)

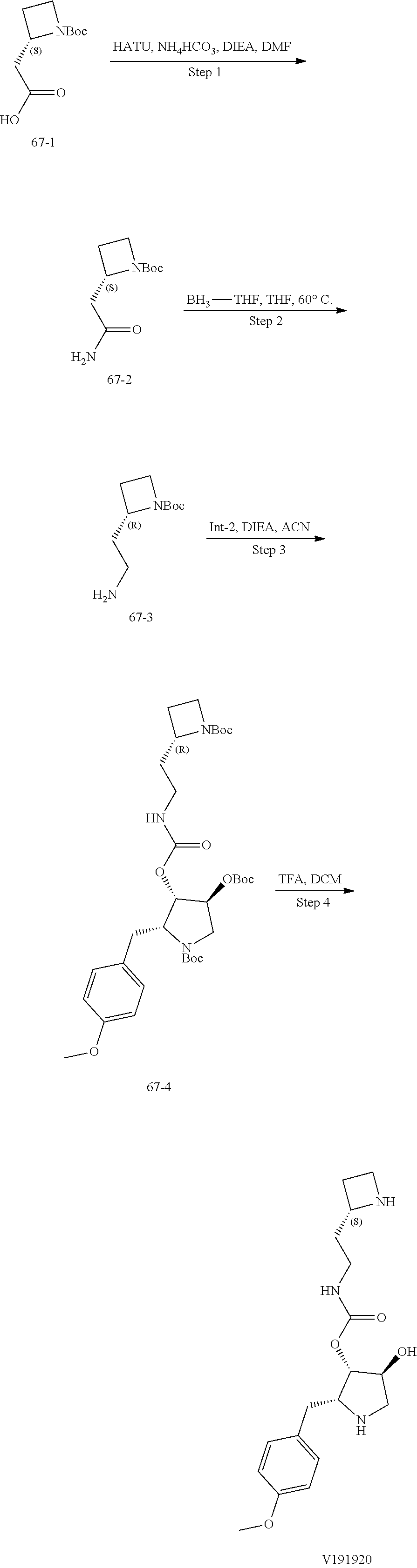

Step 1: To the solution of [1-(tert-butoxycarbonyl)azetidin-2-yl]acetic acid (64 mg, 0.30 mmol, 1 equiv) and NH₄HCO₃ (47 mg, 0.59 mmol, 2 equiv) in DMF (2 mL) was added DIEA (77 mg, 0.59 mmol, 2 equiv) and HATU (136 mg, 0.36 mmol, 1.2 equiv). The mixture was stirred at rt for overnight. The mixture was diluted with EA, wash with water, dried and concentrated to give the crude product which was used in the next step without further purification. The tert-butyl 2-(carbamoylmethyl)azetidine-1-carboxylate (40 mg, 62.8%) was obtained as a yellow oil. MS: m/z: Calc'd for C₁₀H₁₈N₂O₃[M+H]⁺215. found 215.

Step 2: To the solution of tert-butyl 2-(carbamoylmethyl)azetidine-1-carboxylate (40 mg, 0.19 mmol, 1 equiv) in THF (5 mL) was added BH₃-THF (160 mg, 1.87 mmol, 10 equiv). The mixture was stirred at rt for 3 hours at 60° C. under N₂. The mixture was concentrated to give the crude product which was used in the next step without further purification. The tert-butyl (2R)-2-(2-aminoethyl)azetidine-1-carboxylate (30 mg, 80.2%) was obtained as a colorless oil. MS: m/z: Calc'd for C₁₀H₂₀N₂O₂ [M+H]⁺201. found 201.

### Step 3 and 4

The title compound was prepared in 17.2% overall yield as a grey solid according to Substitution reaction for synthesis of Carbamate; General Procedure I using tert-butyl (2R)-2-(2-aminoethyl)azetidine-1-carboxylate (67-3) in STEP 3; Boc Deprotection; General Procedure II in STEP 4. MS: m/z Calc'd for C₁₈H₂₇N₃O₄ [M+H]⁺350. found 350. ¹H NMR (400 MHz, Methanol-d₄) δ 7.30-7.22 (m, 2H), 6.98-6.90 (m, 2H), 4.96 (d, J=3.4 Hz, 1H), 4.59-4.46 (m, 1H), 4.39 (d, J=4.2 Hz, 1H), 4.23-4.14 (m, 1H), 4.14-4.02 (m, 1H), 3.98-3.87 (m, 1H), 3.80 (s, 3H), 3.59 (dd, J=12.7, 4.3 Hz, 1H), 3.34-3.18 (m, 3H), 3.17-3.07 (m, 1H), 3.05-2.95 (m, 1H), 2.70-2.56 (m, 1H), 2.47-2.33 (m, 1H), 2.28-2.03 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 18% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-{2-[(2S)-2-methylpyrrolidin-2-yl]ethyl}carbamate (V191921)

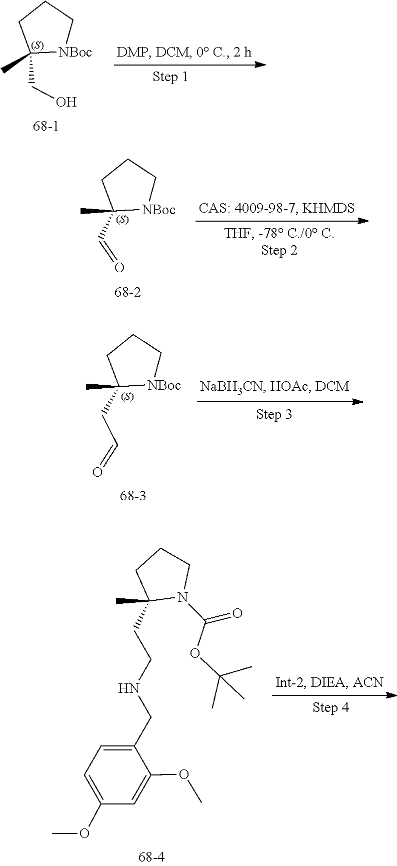

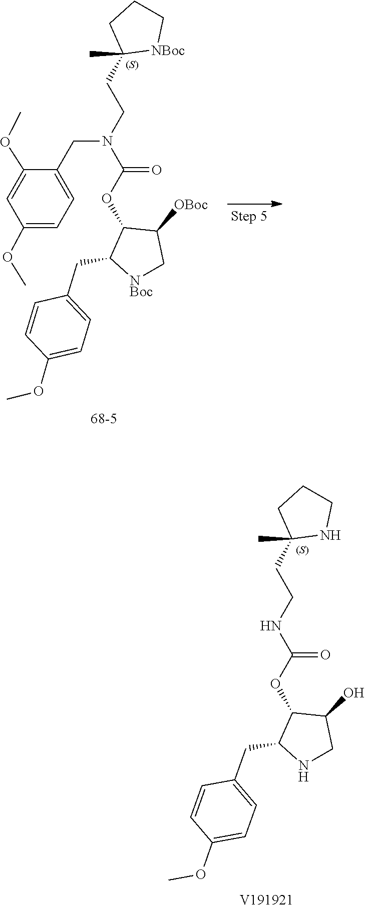

Step 1: To a stirred mixture of tert-butyl (2S)-2-(hydroxymethyl)-2-methylpyrrolidine-1-carboxylate (300 mg, 1.39 mmol, 1 equiv) in DCM (15 mL) was added DMP (650.1 mg, 1.52 mmol, 1.1 equiv) at 0° C. The resulting mixture was stirred at room temperature for 2 h. The residue was purified by Prep-TLC (PE/EA 1:1) to afford tert-butyl (2S)-2-formyl-2-methylpyrrolidine-1-carboxylate (280 mg, 95% purity) as a yellow oil.

Step 2: To a stirred mixture of (Methoxymethyl)triphenylphosphonium chloride (825.3 mg, 2.40 mmol, 2 equiv) in THF (10 mL) was added KHMDS (718.3 mg, 3.60 mmol, 3 equiv) at −78° C. under nitrogen atmosphere. The resulting mixture was stirred at −78° C. for 1 h, and then stirred at 0° C. for another 1 h. To a stirred mixture of crude was added tert-butyl (2S)-2-formyl-2-methylpyrrolidine-1-carboxylate (256 mg, 1.200 mmol, 1 equiv) at 0° C. The resulting mixture was stirred at room temperature for overnight. Upon completion, the mixture was extracted with Et₂O (2×20 mL). The combined organic layers were washed with saturated salt water (1×20 mL), dried over anhydrous Na₂SO₄. After filtration, the filtrate was concentrated under reduced pressure. The residue was dissolved in Et₂O (10 mL). The resulting mixture was stirred at −30° C. for 0.5h under. The resulting mixture was filtered. the filter cake was washed with Et₂O (2×5 mL). The filtrate was concentrated under reduced pressure. The residue was dissolved in acetone (10 mL) and HCl (6M) (21.88 mg, 0.60 mmol, 0.5 equiv). The mixture was stirred at room temperature for 10 min, then extracted with Et₂O (2×20 mL). The combined organic layers were washed with saturated salt water (1×20 mL), dried over anhydrous Na₂SO₄. After filtration, the filtrate was concentrated under reduced pressure to afford crude product tert-butyl (2S)-2-methyl-2-(2-oxoethyl)pyrrolidine-1-carboxylate (150 mg) as a yellow oil. MS: m/z: Calc'd for C₁₂H₂₁NO₃ [M+H]⁺228. found 228.

Step 3: A solution of tert-butyl (2S)-2-methyl-2-(2-oxoethyl)pyrrolidine-1-carboxylate (150 mg, 0.66 mmol, 1 equiv) and 1-(2,4-dimethoxyphenyl)methanamine (165.5 mg, 0.99 mmol, 1.5 equiv) in DCM (5 mL) was treated with HOAc (79.2 mg, 1.32 mmol, 2 equiv) at 0° C. for 30 min under nitrogen atmosphere followed by the addition of NaBH₃CN (82.9 mg, 1.32 mmol, 2 equiv) at room temperature. The resulting mixture was stirred at room temperature for overnight. The resulting mixture was concentrated under reduced pressure. The residue was purified by reversed-phase flash chromatography to afford tert-butyl (2S)-2-(2-{[(2,4-dimethoxyphenyl)methyl]amino}ethyl)-2-methylpyrrolidine-1-carboxylate (230 mg, 92.8%) as a white solid. MS: m/z: Calc'd for C₂₁H₃₄N₂O₄ [M+22]⁺401. found 401.

Step 4 and 5: The title compound was prepared in 70.1% overall yield as an off-white solid according to Substitution reaction for synthesis of Carbamate; General Procedure I using tert-butyl (2S)-2-(2-{[(2,4-dimethoxyphenyl)methyl]amino}ethyl)-2-methylpyrrolidine-1-carboxylate (68-4) in STEP 4; Boc Deprotection; General Procedure II in STEP 5. MS: m/z Calc'd for C₂₀H₃₁N₃O₄ [M+H]⁺378. found 378. ¹H NMR (400 MHz, Methanol-d₄) δ 7.29-7.19 (m, 2H), 6.96-6.88 (m, 2H), 5.00-4.94 (m, 1H), 4.38 (d, J=4.2 Hz, 1H), 4.21-4.12 (m, 1H), 3.79 (s, 3H), 3.58 (dd, J=12.6, 4.3 Hz, 1H), 3.46-3.34 (m, 2H), 3.29 (dd, J=7.9, 3.5 Hz, 2H), 3.22 (d, J=12.7 Hz, 1H), 3.11 (dd, J=14.1, 7.1 Hz, 1H), 2.98 (dd, J=14.2, 8.5 Hz, 1H), 2.24-2.08 (m, 2H), 2.07-1.92 (m, 4H), 1.44 (s, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-{2-[(5S)-4-azaspiro[2.4]heptan-5-yl]ethyl}carbamate (V191945)

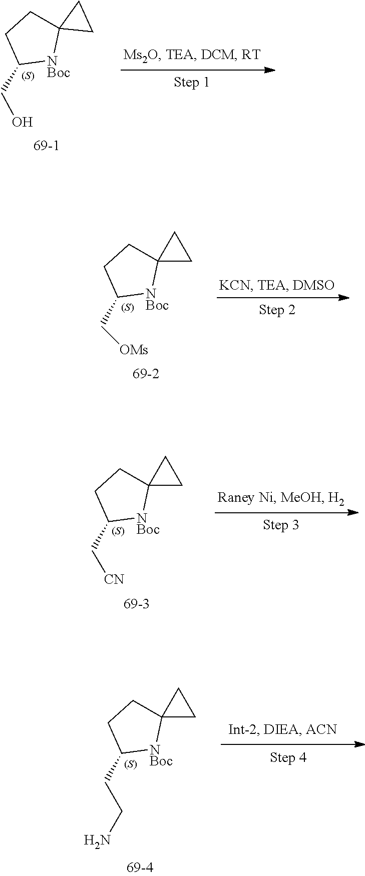

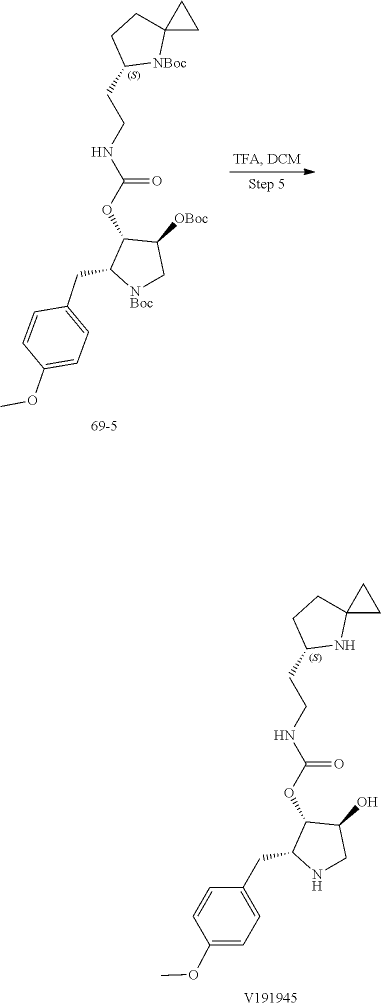

Step 1: To the solution of tert-butyl (5S)-5-(hydroxymethyl)-4-azaspiro[2.4]heptane-4-carboxylate (290 mg, 1.28 mmol, 1 equiv) and methanesulfonyl methanesulfonate (333 mg, 1.91 mmol, 1.5 equiv) in DCM (4 mL) was added TEA (258 mg, 2.55 mmol, 2 equiv) slowly at 0° C. The mixture was stirred at rt for overnight. The mixture was diluted with DCM, washed with water, dried and concentrated to give the crude product which was used in the next step directly as a yellow oil. MS: m/z: Calc'd for C₁₃H₂₃NO₅S [M+H−56]⁺250. found 250.

Step 2: To the solution of tert-butyl (5S)-5-[(methanesulfonyloxy)methyl]-4-azaspiro[2.4]heptane-4-carboxylate (300 mg, 0.98 mmol, 1 equiv) in DMSO (5 mL) was added DIEA (254 mg, 1.96 mmol, 2 equiv) and KCN (128 mg, 1.96 mmol, 2 equiv). The mixture was stirred at 80° C. for overnight. The mixture was diluted with EA, washed with water, dried and concentrated to give the crude product which was used in the next step without further purification. The tert-butyl (5S)-5-(cyanomethyl)-4-azaspiro[2.4]heptane-4-carboxylate (180 mg, 77.5%) was obtained as a brown oil. MS: m/z: Calc'd for C₁₃H₂₀N₂O₂ [M+H−56]⁺181. found 181.

Step 3: To the solution of tert-butyl (5S)-5-(cyanomethyl)-4-azaspiro[2.4]heptane-4-carboxylate (190 mg, 0.80 mmol, 1 equiv) in MeOH (5 mL) was added Raney-Ni (190 mg, 2.22 mmol, 2.76 equiv). The mixture was stirred at rt for overnight under H2. The mixture was filtered and the filtrate was concentrated to give the crude product which was used directly in the next step. The tert-butyl (5S)-5-(2-aminoethyl)-4-azaspiro[2.4]heptane-4-carboxylate (120 mg, 62.1%) was obtained as a colorless oil. MS: m/z: Calc'd for C₁₃H₂₄N₂O₂ [M+H]⁺241. found 241.

Step 4 and 5: The title compound was prepared in 24.8% overall yield as a white solid according to Substitution reaction for synthesis of Carbamate; General Procedure I using tert-butyl (5S)-5-(2-aminoethyl)-4-azaspiro[2.4]heptane-4-carboxylate (69-4) in STEP 4; Boc Deprotection; General Procedure II in STEP 5. MS: m/z Calc'd for C21H₃₁N₃O₄[M+H]⁺390. found 390. ¹H NMR (400 MHz, Methanol-d₄) δ 7.29-7.21 (m, 2H), 6.99-6.89 (m, 2H), 4.99-4.94 (m, 1H), 4.41-4.36 (m, 1H), 4.24-4.15 (m, 1H), 3.80 (s, 3H), 3.81-3.71 (m, 1H), 3.58 (dd, J=12.6, 4.3 Hz, 1H), 3.33-3.19 (m, 3H), 3.12 (dd, J=14.1, 7.2 Hz, 1H), 3.00 (dd, J=14.1, 8.5 Hz, 1H), 2.50-2.37 (m, 1H), 2.14 (dd, J=9.1, 5.8 Hz, 2H), 2.10-1.87 (m, 3H), 1.27-1.11 (m, 2H), 1.05-0.96 (m, 1H), 0.96-0.85 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 18% B in 10 min; Wave Length: 254 nm/220 nm; RT1(min): 8.5.

### Synthetic Procedure of (2R,3S,4S)-4-hydroxy-2-(4-methoxybenzyl)pyrrolidin-3-yl (2-((R)-2-methylpyrrolidin-2-yl)ethyl)carbamate (V192041)

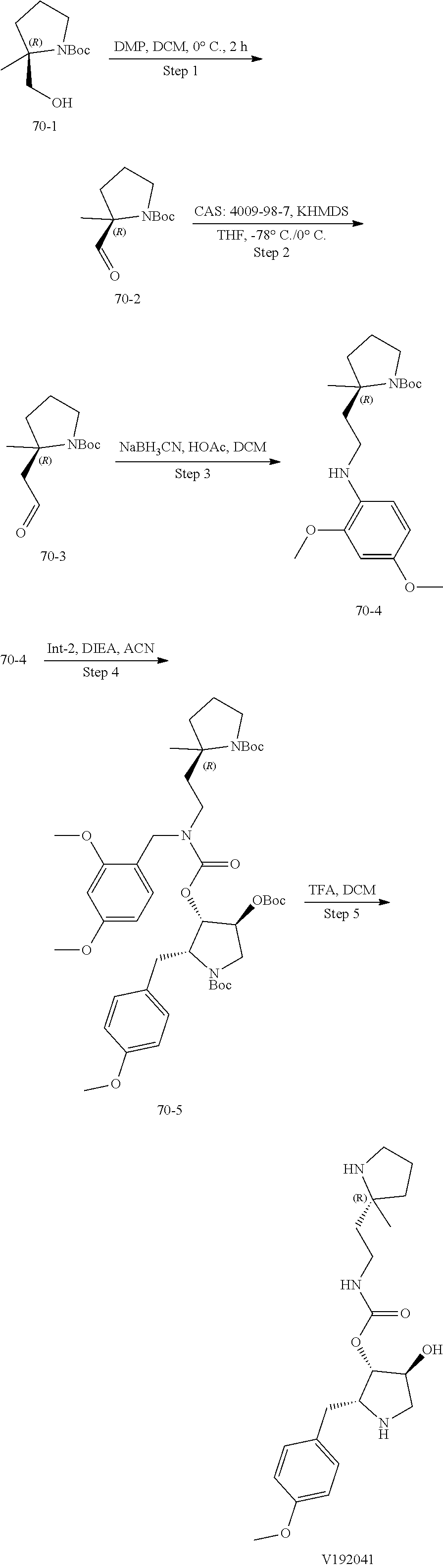

Step 1: To a stirred mixture of tert-butyl (2R)-2-(hydroxymethyl)-2-methylpyrrolidine-1-carboxylate (500 mg, 2.32 mmol, 1 equiv) in DCM (15 mL) was added 1,1-bis(acetyloxy)-3-oxo-3H-1l\hat{ }[5],2-benziodoxol-1-yl acetate (1083.54 mg, 2.554 mmol, 1.1 equiv) at 0° C. The resulting mixture was stirred at room temperature for 2 h. The resulting mixture was filtered, the filter cake was washed with DCM (2×2 mL). The filtrate was concentrated under reduced pressure. This resulted in tert-butyl (2R)-2-formyl-2-methylpyrrolidine-1-carboxylate (490 mg) as a yellow oil.

Step 2: To a stirred mixture of (Methoxymethyl)triphenylphosphonium chloride (967.2 mg, 2.81 mmol, 2 equiv) in THF (10 mL) was added KHMDS (841.8 mg, 4.22 mmol, 3 equiv) at −78° C. under nitrogen atmosphere. The resulting mixture was stirred at −78° C. for 1 h. The resulting mixture was stirred at 0° C. for another 1 h. To the above mixture was added tert-butyl (2R)-2-formyl-2-methylpyrrolidine-1-carboxylate (300 mg, 1.40 mmol, 1 equiv) at 0° C. The resulting mixture was stirred at room temperature for overnight. Upon completion, the resulting mixture was extracted with Et₂O (2×20 mL). The combined organic layers were washed with saturated brine (1×20 mL), dried over anhydrous Na₂SO₄. After filtration, the filtrate was concentrated under reduced pressure. The residue was dissolved in Et₂O (10 mL). The resulting mixture was stirred at −30° C. for 0.5h under. The resulting mixture was filtered. the filter cake was washed with Et₂O (2×5 mL). The filtrate was concentrated under reduced pressure. The residue was dissolved in acetone (10 mL) was added HCl (6M) (25.6 mg, 0.70 mmol, 0.5 equiv) at room temperature. The resulting mixture was stirred at room temperature for 10 min. The resulting mixture was extracted with Et₂O (2×20 mL). The combined organic layers were washed with saturated salt water (1×20 mL), dried over anhydrous Na₂SO₄. After filtration, the filtrate was concentrated under reduced pressure. This resulted in tert-butyl (2R)-2-methyl-2-(2-oxoethyl)pyrrolidine-1-carboxylate (200 mg, 62.5% yield, 90% purity) as a yellow oil. MS: m/z: Calc'd for C₁₂H₂₁NO₃ [M+H]+ 228. found 228.

Step 3: A solution of tert-butyl (2R)-2-methyl-2-(2-oxoethyl)pyrrolidine-1-carboxylate (200 mg, 0.88 mmol, 1 equiv) and 1-(2,4-dimethoxyphenyl)methanamine (294.2 mg, 1.76 mmol, 2 equiv) in DCM (5 mL) was treated with HOAc (105.6 mg, 1.76 mmol, 2 equiv) at 0° C. for 30 min followed by the addition of NaBH₃CN (110.5 mg, 1.76 mmol, 2 equiv) at room temperature. The resulting mixture was stirred at room temperature for overnight. The resulting mixture was concentrated under reduced pressure. The residue was purified by reversed-phase flash chromatography to afford tert-butyl (2R)-2-{2-[(2,4-dimethoxyphenyl)amino]ethyl}-2-methylpyrrolidine-1-carboxylate (250 mg, 77.9% yield, 85% purity) as a yellow solid. MS: m/z: Calc'd for C₂₀H₃₂N₂O₄ [M+H]⁺365. found 365.

### Step 4 and 5

The title compound was prepared in 21.5% overall yield as a white solid according to Substitution reaction for synthesis of Carbamate; General Procedure I using tert-butyl (2R)-2-{2-[(2,4-dimethoxyphenyl)amino]ethyl}-2-methylpyrrolidine-1-carboxylate (70-4) in STEP 4; Boc Deprotection; General Procedure II in STEP 5. MS: m/z Calc'd for C₂₀H₃₁N₃O₄ [M+H]⁺378. found 378. ¹H NMR (400 MHz, Methanol-d₄) δ 7.27-7.19 (m, 2H), 6.96-6.88 (m, 2H), 4.99-4.93 (m, 1H), 4.37 (d, J=4.2 Hz, 1H), 4.20-4.10 (m, 1H), 3.79 (s, 3H), 3.56 (dd, J=12.6, 4.3 Hz, 1H), 3.46-3.33 (m, 3H), 3.29 (dd, J=7.7, 3.7 Hz, 1H), 3.20 (d, J=12.6 Hz, 1H), 3.10 (dd, J=14.1, 7.1 Hz, 1H), 2.96 (dd, J=14.1, 8.5 Hz, 1H), 2.24-2.09 (m, 2H), 2.07-1.92 (m, 4H), 1.44 (s, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 9.7.

### Synthetic Procedure of (2R,3S,4S)-2-{[4-(2,2-difluoro-1,3-dihydroinden-5-yl)phenyl]methyl}-4-hydroxypyrrolidin-3-yl N-(1,2,3,4-tetrahydroisoquinolin-6-ylmethyl)carbamate (V192060)

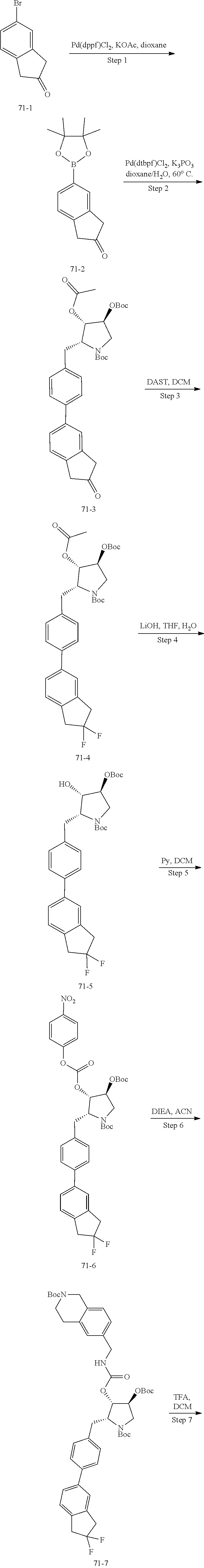

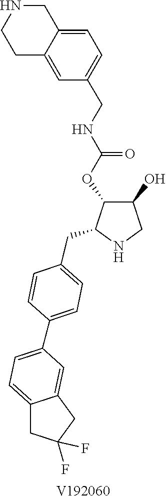 [SMILES: O=C(NCc1ccc2c(c1)CCNC2)O[C@@H]1[C@@H](O)CN[C@@H]1Cc1ccc(-c2ccc3c(c2)CC(F)(F)C3)cc1]

Step 1: To the solution of 5-bromo-1,3-dihydroinden-2-one (1 g, 4.74 mmol, 1 equiv) and bis(pinacolato)diboron (1.80 g, 7.11 mmol, 1.5 equiv) in dioxane (20 mL) was added KOAc (1395 mg, 14.21 mmol, 3 equiv) and Pd(dppf)Cl₂·CH₂Cl₂ (386 mg, 0.47 mmol, 0.1 equiv). The mixture was stirred at 80° C. for overnight under N₂. The mixture was concentrated to give the crude product which was purified by silica gel column chromatography, eluted with PE:EA (5:1) to afford 5-(4,4,5,5-tetramethyl-1,3,2-dioxaborolan-2-yl)-1,3-dihydroinden-2-one (800 mg, 65.4%) as a light yellow solid. MS: m/z: Calc'd for C₁₅H₁₉BO₃ [M+H]⁺259. found 259.

Step 2: To the solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (300 mg, 0.51 mmol, 1 equiv), and 5-(4,4,5,5-tetramethyl-1,3,2-dioxaborolan-2-yl)-1,3-dihydroinden-2-one (199 mg, 0.77 mmol, 1.5 equiv) in dioxane (3 mL) and water (0.3 mL) was added Pd(dppf)Cl₂·CH₂Cl₂ (42 mg, 0.051 mmol, 0.1 equiv) and K₃PO₄ (218 mg, 1.03 mmol, 2 equiv). The mixture was stirred at 60° C. for 6 hours under N₂. The mixture was diluted with EA, washed with water, dried and concentrated to give the crude product which was purified by Prep TLC (PE:EA=3:1). The tert-butyl (2R,3S,4S)-3-(acetyloxy)-4-[(tert-butoxycarbonyl)oxy]-2-{[4-(2-oxo-1,3-dihydroinden-5-yl)phenyl]methyl}pyrrolidine-1-carboxylate (280 mg, 96.3%) was obtained as a brown oil. MS: m/z: Calc'd for C₃₂H₃₉NO₈ [M+H−56−56]⁺454. found 454.

Step 3: To the solution of tert-butyl (2R,3S,4S)-3-(acetyloxy)-4-[(tert-butoxycarbonyl)oxy]-2-{[4-(2-oxo-1,3-dihydroinden-5-yl)phenyl]methyl}pyrrolidine-1-carboxylate (310 mg, 0.55 mmol, 1 equiv) in DCM (3 mL) was added DAST (1 mL, 3.89 mmol). The mixture was stirred at rt for 2 days. The mixture was concentrated. The residue was dissolved in DCM, washed with water, dried and concentrated to give the crude product which was used directly in the next step. MS: m/z: Calc'd for C₃₂H₃₉F₂NO₇ [M+H−56−56]⁺476. found 476.

Step 4: The title compound was prepared in 80.8% yield as a brown oil according to Hydrolysis reaction with LiOH; General Procedure V using tert-butyl (2R,3S,4S)-3-(acetyloxy)-4-[(tert-butoxycarbonyl)oxy]-2-{[4-(2,2-difluoro-1,3-dihydroinden-5-yl)phenyl]methyl}pyrrolidine-1-carboxylate (71-4). MS: m/z: Calc'd for C₃₀H₃₇F₂NO₆ [M+Na-100]⁺468. found 468.

Step 5: To the solution of tert-butyl (2R,3S,4S)-4-[(tert-butoxycarbonyl)oxy]-2-{[4-(2,2-difluoro-1,3-dihydroinden-5-yl)phenyl]methyl}-3-hydroxypyrrolidine-1-carboxylate (150 mg, 0.27 mmol, 1 equiv) and 4-nitrophenyl carbonochloridate (111 mg, 0.55 mmol, 2 equiv) in DCM (3 mL) was added Pyridine (43 mg, 0.55 mmol, 2 equiv). The mixture was stirred at rt for overnight. The mixture was concentrated to give the crude product which was purified by reversed phase flash column to afford tert-butyl (2R,3S,4S)-4-[(tert-butoxycarbonyl)oxy]-2-{[4-(2,2-difluoro-1,3-dihydroinden-5-yl)phenyl]methyl}-3-[(4-nitrophenoxycarbonyl)oxy]pyrrolidine-1-carboxylate (60 mg, 30.7%) as a brown oil. MS: m/z: Calc'd for C₃₀H₃₇F₂NO₆ [M+H−100]⁺611. found 611.

### Step 6 and 7

The title compound was prepared in 58.4% overall yield as a white solid according to Substitution reaction for synthesis of Carbamate; General Procedure I using tert-butyl (2R,3S,4S)-4-[(tert-butoxycarbonyl)oxy]-2-{[4-(2,2-difluoro-1,3-dihydroinden-5-yl)phenyl]methyl}-3-[(4-nitrophenoxycarbonyl)oxy]pyrrolidine-1-carboxylate (71-6) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 6-(aminomethyl)-3,4-dihydro-1H-isoquinoline-2-carboxylate in STEP 6; Boc Deprotection; General Procedure II in STEP 7. MS: m/z Calc'd for C₃₁H₃₃F₂N₃O₃[M+H]⁺534. found 534. ¹H NMR (400 MHz, Methanol-d₄) δ 7.63-7.57 (m, 2H), 7.54-7.46 (m, 2H), 7.43-7.32 (m, 3H), 7.30-7.19 (m, 3H), 5.03-4.98 (m, 1H), 4.45-4.36 (m, 2H), 4.36-4.30 (m, 3H), 4.30-4.22 (m, 1H), 3.67-3.58 (m, 1H), 3.56-3.42 (m, 6H), 3.29-3.17 (m, 2H), 3.15-3.05 (m, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 8.5.

### Synthetic Procedure of (2R,3S,4S)-2-{[4-(2,2-difluoro-1,3-dihydroinden-5-yl)phenyl]methyl}-4-hydroxypyrrolidin-3-yl N-{2-[(2S)-pyrrolidin-2-yl]ethyl}carbamate (V191961)

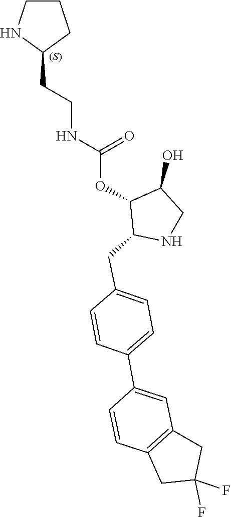

The title compound was prepared in 64.2% overall yield as a white solid according to Substitution reaction for synthesis of Carbamate; General Procedure I using tert-butyl (2R,3S,4S)-4-[(tert-butoxycarbonyl)oxy]-2-{[4-(2,2-difluoro-1,3-dihydroinden-5-yl)phenyl]methyl}-3-[(4-nitrophenoxycarbonyl)oxy]pyrrolidine-1-carboxylate (71-6) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate in STEP 1; Boc Deprotection; General Procedure II in STEP 2. MS: m/z Calc'd for C₂₇H₃₃F₂N₃O₃[M+H]⁺486. found 486. ¹H NMR (400 MHz, Methanol-d₄) δ 7.67-7.60 (m, 2H), 7.51 (d, J=8.3 Hz, 2H), 7.42 (d, J=8.0 Hz, 2H), 7.34 (d, J=7.8 Hz, 1H), 5.04-4.99 (m, 1H), 4.41 (d, J=4.1 Hz, 1H), 4.32-4.22 (m, 1H), 3.69-3.54 (m, 2H), 3.54-3.37 (m, 4H), 3.34-3.18 (m, 6H), 3.16-3.06 (m, 1H), 2.35-2.22 (m, 1H), 2.21-1.87 (m, 4H), 1.78-1.63 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 11% B to 41% B in 10 min; Wave Length: 254 nm/220 nm; RT1(min): 9.2.

### Synthetic Procedure of (2R,3S,4S)-2-{[4-(2,2-difluoro-1,3-dihydroinden-5-yl)phenyl]methyl}-4-hydroxypyrrolidin-3-yl N-{2-[(2R)-pyrrolidin-2-yl]ethyl}carbamate (V192040)

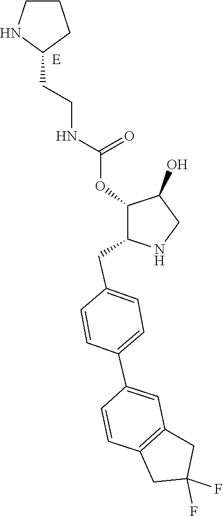

The title compound was prepared in 65.5% overall yield as a white solid according to Substitution reaction for synthesis of Carbamate; General Procedure I using tert-butyl (2R,3S,4S)-4-[(tert-butoxycarbonyl)oxy]-2-{[4-(2,2-difluoro-1,3-dihydroinden-5-yl)phenyl]methyl}-3-[(4-nitrophenoxycarbonyl)oxy]pyrrolidine-1-carboxylate (71-6) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2R)-2-(2-aminoethyl)pyrrolidine-1-carboxylate in STEP 1; Boc Deprotection; General Procedure II in STEP 2. MS: m/z Calc'd for C₂₇H₃₃F₂N₃O₃[M+H]⁺486. found 486. ¹H NMR (400 MHz, Methanol-d₄) δ 7.63 (d, J=8.1 Hz, 2H), 7.50 (d, J=8.3 Hz, 2H), 7.41 (d, J=7.9 Hz, 2H), 7.34 (d, J=7.8 Hz, 1H), 5.02 (d, J=3.5 Hz, 1H), 4.41 (d, J=4.2 Hz, 1H), 4.27-4.23 (m, 1H), 3.69-3.54 (m, 2H), 3.57-3.48 (m, 2H), 3.48-3.41 (m, 2H), 3.33-3.17 (m, 6H), 3.14-3.04 (m, 1H), 2.37-2.24 (m, 1H), 2.16-1.87 (m, 4H), 1.78-1.64 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 12% B to 42% B in 10 min; Wave Length: 254 nm/220 nm; RT1(min): 9.1.

### Synthetic Procedure of (2R,3S,4S)-2-{[4-(2,2-difluoro-1,3-dihydroinden-5-yl)phenyl]methyl}-4-hydroxypyrrolidin-3-yl N-[2-(piperidin-4-yl)ethyl]carbamate (V192053)

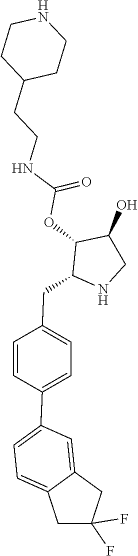 [SMILES: O=C(NCCC1CCNCC1)O[C@@H]1[C@@H](O)CN[C@@H]1Cc1ccc(-c2ccc3c(c2)CC(F)(F)C3)cc1]

The title compound was prepared in 65.5% overall yield as a white solid according to Substitution reaction for synthesis of Carbamate; General Procedure I using tert-butyl (2R,3S,4S)-4-[(tert-butoxycarbonyl)oxy]-2-{[4-(2,2-difluoro-1,3-dihydroinden-5-yl)phenyl]methyl}-3-[(4-nitrophenoxycarbonyl)oxy]pyrrolidine-1-carboxylate (71-6) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 4-(2-aminoethyl)piperidine-1-carboxylate in STEP 1; Boc Deprotection; General Procedure II in STEP 2. MS: m/z Calc'd for C₂₈H₃₅F₂N₃O₃[M+H]⁺500. found 500. ¹H NMR (400 MHz, Methanol-d₄) δ 7.67-7.60 (m, 2H), 7.50 (d, J=8.5 Hz, 2H), 7.44-7.38 (m, 2H), 7.34 (d, J=7.8 Hz, 1H), 5.03-4.98 (m, 1H), 4.40 (d, J=4.2 Hz, 1H), 4.29-4.20 (m, 1H), 3.69-3.55 (m, 1H), 3.55-3.43 (m, 4H), 3.44-3.35 (m, 2H), 3.30-3.18 (m, 4H), 3.14-3.04 (m, 1H), 3.03-2.91 (m, 2H), 2.05-1.96 (m, 2H), 1.76-1.65 (m, 1H), 1.62-1.52 (m, 2H), 1.46-1.33 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 11% B to 41% B in 10 min; Wave Length: 254 nm/220 nm; RT1(min): 9.2.

### Synthetic Procedure of (2R,3S,4S)-4-hydroxy-2-{[4-(1,3-thiazol-5-yl)phenyl]methyl}pyrrolidin-3-yl N-{2-[(2S)-pyrrolidin-2-yl]ethyl}carbamate (V192113)

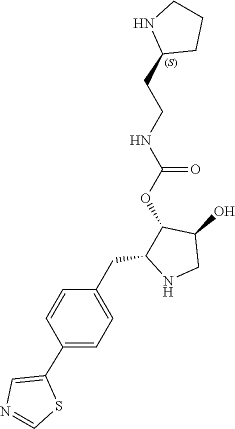

The title compound was prepared in 11.2% overall yield as an off-white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-thiazol-5-yl)phenyl]methyl}pyrrolidine-1-carboxylate (Int-13) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate in STEP 1; Boc Deprotection; General Procedure II in STEP 2. MS: m/z Calc'd for C₂₁H₂₈N₄O₃S [M+H]⁺417. found 417. ¹H NMR (400 MHz, Methanol-d₄) δ 9.00 (s, 1H), 8.22-8.16 (m, 1H), 7.73-7.67 (m, 2H), 7.50-7.40 (m, 2H), 5.02 (d, J=3.4 Hz, 1H), 4.41 (d, J=4.1 Hz, 1H), 4.29 (dd, J=58.3, 44.8 Hz, 1H), 3.65-3.50 (m, 2H), 3.33 (s, 2H), 3.32-3.18 (m, 4H), 3.10 (dd, J=14.2, 8.7 Hz, 1H), 2.36-2.23 (m, 1H), 2.19-1.85 (m, 4H), 1.78-1.64 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 15% B in 13 min; Wave Length: 254 nm/220 nm; RT1(min): 13.9.

### Synthetic Procedure of (2R,3S,4S)-4-hydroxy-2-[(4-methoxyphenyl)methyl]pyrrolidin-3-yl N-{2-[(5R)-4-azaspiro[2.4]heptan-5-yl]ethyl}carbamate (V192043)

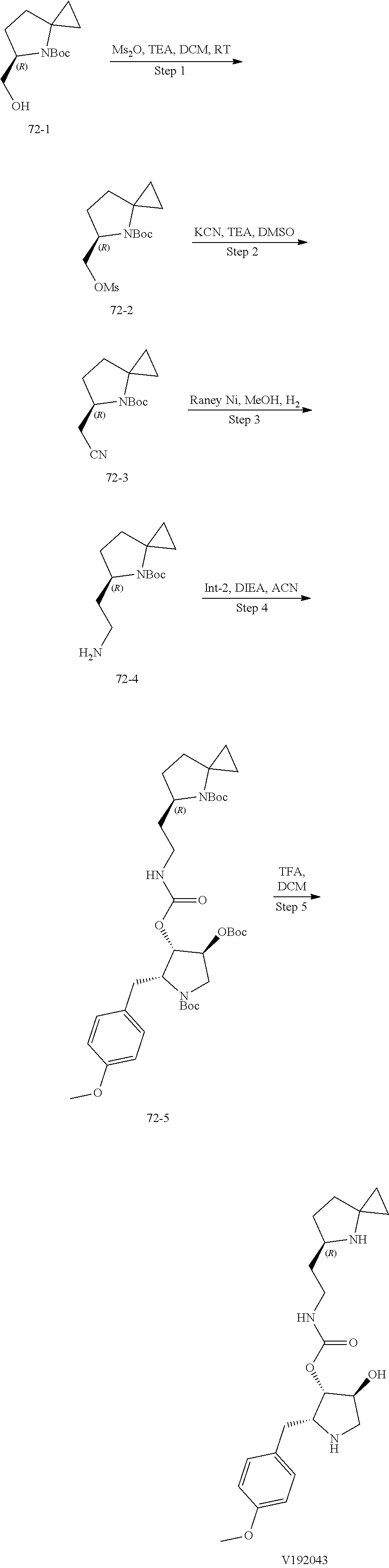

Step 1: To a solution of tert-butyl (5R)-5-(hydroxymethyl)-4-azaspiro[2.4]heptane-4-carboxylate (250 mg, 1.10 mmol, 1 equiv) and methanesulfonyl methanesulfonate (287 mg, 1.65 mmol, 1.5 equiv) in DCM (5 mL) was added TEA (223 mg, 2.20 mmol, 2 equiv) slowly at 0° C. The mixture was stirred at rt for overnight. The mixture was diluted with DCM, washed with water, dried and concentrated to give the crude product which was used in the next step directly as a brown oil. MS: m/z: Calc'd for C₁₃H₂₃NO₅S [M+H−56]⁺250. found 250.

Step 2: To the solution of tert-butyl (5R)-5-[(methanesulfonyloxy)methyl]-4-azaspiro[2.4]heptane-4-carboxylate (320 mg, 1.05 mmol, 1 equiv) in DMSO (5 mL) was added DIEA (271 mg, 2.10 mmol, 2 equiv) and KCN (136 mg, 2.10 mmol, 2 equiv). The mixture was stirred at 80° C. for overnight. The mixture was diluted with EA, washed with water, dried and concentrated to give the crude product which was used in the next step without further purification as a brown oil. MS: m/z: Calc'd for C₁₃H₂₀N₂O₂ [M+H−56]⁺181. found 181.

Step 3: To the solution of tert-butyl (5R)-5-(cyanomethyl)-4-azaspiro[2.4]heptane-4-carboxylate (200 mg, 0.85 mmol, 1 equiv) in MeOH (5 mL) was added Raney-Ni (100 mg, 1.17 mmol, 1.38 equiv). The mixture was stirred at rt for overnight under H₂. The mixture was filtered and the filtrate was concentrated to give the crude product which was used directly in the next step as a colorless oil. MS: m/z: Calc'd for C₁₃H₂₄N₂O₂ [M+H]⁺241. found 241.

### Step 4 and 5

The title compound was prepared in 46.0% overall yield as a white solid according to Substitution reaction for synthesis of Carbamate; General Procedure I using tert-butyl (5R)-5-(2-aminoethyl)-4-azaspiro[2.4]heptane-4-carboxylate (72-4) in STEP 4; Boc Deprotection; General Procedure II in STEP 5. MS: m/z Calc'd for C₂₁H₃₁N₃O₄ [M+H]⁺390. found 390. ¹H NMR (400 MHz, Methanol-d₄) 6.27-7.20 (m, 2H), 6.97-6.89 (m, 2H), 4.97 (d, J=3.5 Hz, 1H), 4.39 (d, J=4.2 Hz, 1H), 4.22-4.12 (m, 1H), 3.87-3.79 (m, 1H), 3.80 (s, 3H), 3.59 (dd, J=12.7, 4.4 Hz, 1H), 3.32-3.25 (m, 2H), 3.22 (d, J=12.7 Hz, 1H), 3.11 (dd, J=14.2, 7.2 Hz, 1H), 2.98 (dd, J=14.1, 8.5 Hz, 1H), 2.50-2.37 (m, 1H), 2.14 (dd, J=8.8, 6.1 Hz, 2H), 2.10-1.87 (m, 3H), 1.27-1.14 (m, 2H), 1.09-0.97 (m, 1H), 0.97-0.86 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 9.5.

### Synthetic Procedure of (2R,3S,4S)-4-hydroxy-2-(4-methoxybenzyl)pyrrolidin-3-yl ((2-(3-hydroxyazetidin-1-yl)pyridin-4-yl)methyl)carbamate (V192067)

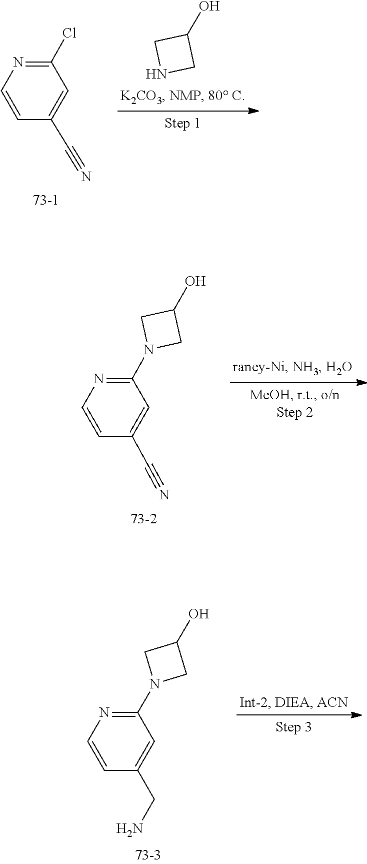 [SMILES: N#Cc1ccnc(Cl)c1 ; OC1CNC1 ; N#Cc1ccnc(N2CC(O)C2)c1 ; NCc1ccnc(N2CC(O)C2)c1]

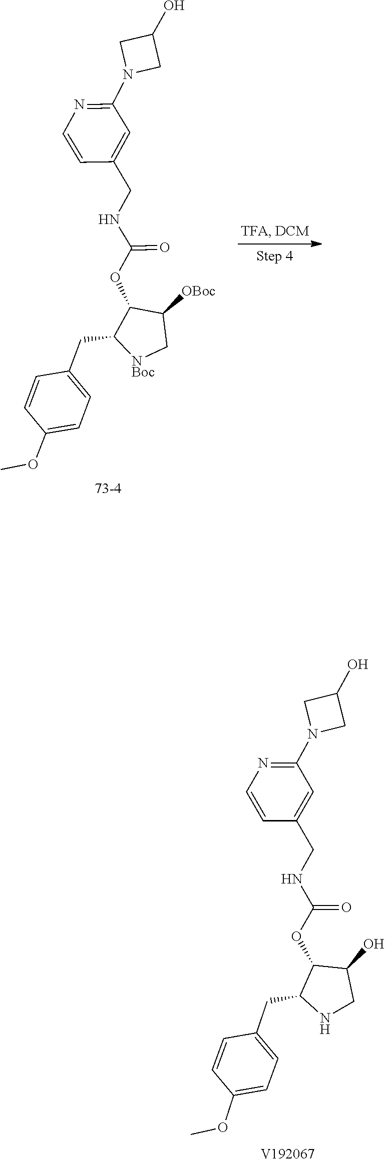

Step 1: To a stirred mixture of 2-chloropyridine-4-carbonitrile (500 mg, 3.69 mmol, 1 equiv) and azetidin-3-ol (395.6 mg, 5.413 mmol, 1.5 equiv) in NMP (10 mL) was added K₂CO₃ (997.5 mg, 7.21 mmol, 2 equiv) at room temperature. The resulting mixture was stirred at 80° C. for overnight. The mixture was allowed to cool down to room temperature. The reaction mixture was quenched with water and extracted with EA. The extracts were washed with brine, dried over anhydrous sodium sulfate, filtered and concentrated. The residue was purified by reversed-phase flash to afford 2-(3-hydroxyazetidin-1-yl)pyridine-4-carbonitrile (260 mg, 41.1%) as a yellow oil. MS: m/z: Calc'd for C₉H₉N₃O [M+H]⁺176. found 176.

Step 2: To a stirred mixture of 2-(3-hydroxyazetidin-1-yl)pyridine-4-carbonitrile (260 mg, 1.44 mmol, 1 equiv) and NH3·H₂O (26.0 mg, 0.74 mmol, 0.5 equiv) in MeOH (10 mL) was added Raney Nickel (174.2 mg, 2.96 mmol, 2 equiv) at room temperature under nitrogen atmosphere. The resulting mixture was stirred at room temperature for overnight under hydrogen atmosphere. The resulting mixture was filtered, the filter cake was washed with MeOH (2×2 mL). The filtrate was concentrated under reduced pressure. This resulted in 1-[4-(aminomethyl)pyridin-2-yl]azetidin-3-ol (210 mg, 78.9%) as a yellow oil. MS: m/z: Calc'd for C₉H₁₃N₃O [M+H]⁺180. found 180.

Step 3 and 4: The title compound was prepared in 14.9% overall yield as a white solid according to Substitution reaction for synthesis of Carbamate; General Procedure I using 1-[4-(aminomethyl)pyridin-2-yl]azetidin-3-ol (73-3) in STEP 3; Boc Deprotection; General Procedure II in STEP 4. MS: m/z Calc'd for C₂₂H₂₈N₄O₅ [M+H]⁺429. found 429. ¹H NMR (400 MHz, Methanol-d₄) δ 7.86 (d, J=6.6 Hz, 1H), 7.28-7.19 (m, 2H), 6.97-6.82 (m, 3H), 6.73 (s, 1H), 4.99 (d, J=3.5 Hz, 1H), 4.84-4.75 (m, 1H), 4.57-4.47 (m, 2H), 4.51-4.32 (m, 3H), 4.23-4.14 (m, 1H), 4.13-4.04 (m, 2H), 3.80 (s, 3H), 3.67-3.58 (m, 1H), 3.24 (d, J=12.7 Hz, 1H), 3.13 (dd, J=14.2, 7.1 Hz, 1H), 3.01 (dd, J=14.2, 8.6 Hz, 1H).

Prep-HPLC purification conditions: Column: Xselect CSH Prep C18 OBD Column, 30*150 mm, 5 μm; Mobile Phase A: Water (0.05% TFA), Mobile Phase B: ACN; Flow rate: 60 mL/min; Gradient: 2% B to 18% B in 7 min; Wave Length: 254 nm/220 nm; RT1(min): 6.07.

### Synthetic Procedure of (2R,3S,4S)-4-hydroxy-2-[(4-methoxyphenyl)methyl]pyrrolidin-3-yl N-[(2-{2-azaspiro[3.3]heptan-2-yl}pyridin-4-yl)methyl]carbamate (V192068)

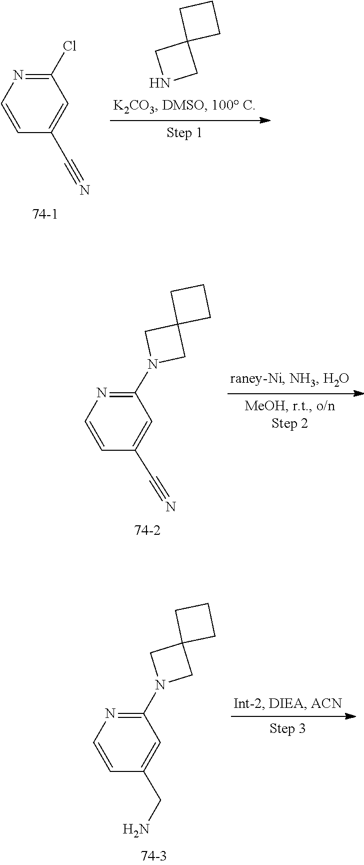 [SMILES: N#Cc1ccnc(Cl)c1 ; C1CC2(C1)CNC2 ; N#Cc1ccnc(N2CC3(CCC3)C2)c1 ; NCc1ccnc(N2CC3(CCC3)C2)c1]

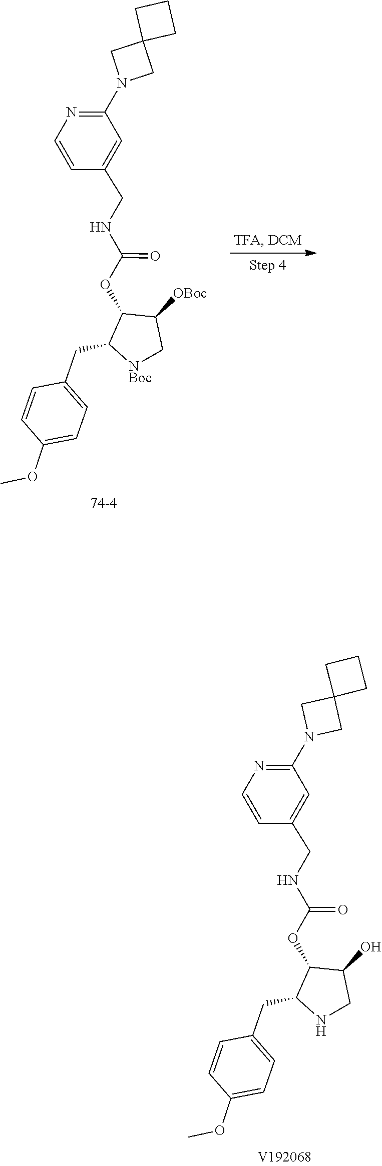

Step 1: To a stirred mixture of 2-chloropyridine-4-carbonitrile (500 mg, 3.60 mmol, 1 equiv) and 2-azaspiro[3.3]heptane (525.9 mg, 5.41 mmol, 1.5 equiv) in DMSO (10 mL) was added K₂CO₃ (1496.2 mg, 10.82 mmol, 3 equiv) at room temperature. The resulting mixture was stirred at room temperature for overnight. The resulting mixture was filtered. the filter cake was washed with DMSO (2×2 mL). The residue was purified by reversed-phase flash chromatography to afford 2-{2-azaspiro[3.3]heptan-2-yl}pyridine-4-carbonitrile (600 mg, 83.4%) as a brown oil. MS: m/z: Calc'd for C₁₂H₁₃N₃ [M+H]+ 200. found 200.

Step 2: To a stirred mixture of 2-{2-azaspiro[3.3]heptan-2-yl}pyridine-4-carbonitrile (600 mg, 3.01 mmol, 1 equiv) in methanol (5 mL) was added raney nickel (176.7 mg, 3.01 mmol, 1 equiv) and Ammonium hydroxide (9% in water) (31.6 mg, 0.90 mmol, 0.3 equiv) at 0° C. under nitrogen atmosphere. The resulting mixture was stirred at room temperature for overnight under hydrogen atmosphere. The resulting mixture was filtered, the filter cake was washed with methanol (1×2 mL). The filtrate was concentrated under reduced pressure. This resulted in 1-(2-{2-azaspiro[3.3]heptan-2-yl}pyridin-4-yl)methanamine (500 mg) as a yellow solid. MS: m/z: Calc'd for C₁₂H₁₇N₃[M+H]+ 204, found 204.

### Step 3 and 4

The title compound was prepared in 28.7% overall yield as a white solid according to Substitution reaction for synthesis of Carbamate; General Procedure I using 1-(2,4-dimethoxyphenyl)methanamine (74-3) in STEP 3; Boc Deprotection; General Procedure II in STEP 4. MS: m/z Calc'd for C₂₅H₃₂N₄O₄ [M+H]⁺453. found 453. ¹H NMR (400 MHz, Methanol-d₄) δ 7.82 (d, J=6.6 Hz, 1H), 7.29-7.22 (m, 2H), 6.97-6.80 (m, 3H), 6.67 (s, 1H), 5.03 (dd, J=3.6, 1.8 Hz, 1H), 4.47-4.31 (m, 3H), 4.29-4.14 (m, 5H), 3.80 (d, J=0.7 Hz, 3H), 3.68-3.59 (m, 1H), 3.24 (d, J=12.7 Hz, 1H), 3.14 (dd, J=14.1, 6.9 Hz, 1H), 3.07-2.95 (m, 1H), 2.28 (dd, J=9.7, 6.8 Hz, 4H), 1.95-1.83 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 4% B to 34% B in 10 min; Wave Length: 254 nm/220 nm; RT1(min): 9.3.

### Synthetic Procedure of (2R,3S,4S)-4-hydroxy-2-[(4-methoxyphenyl)methyl]pyrrolidin-3-yl N-{[4-(1-aminocyclopropyl)phenyl]methyl}carbamate (V192069)

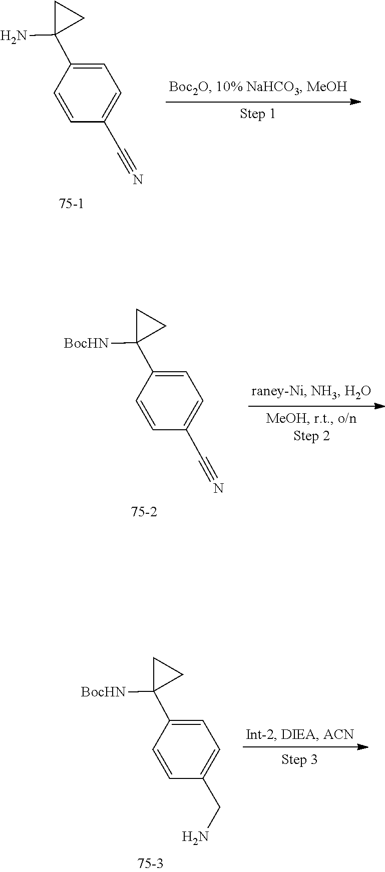 [SMILES: N#Cc1ccc(C2(N)CC2)cc1 ; CC(C)(C)OC(=O)NC1(c2ccc(C#N)cc2)CC1 ; CC(C)(C)OC(=O)NC1(c2ccc(CN)cc2)CC1]

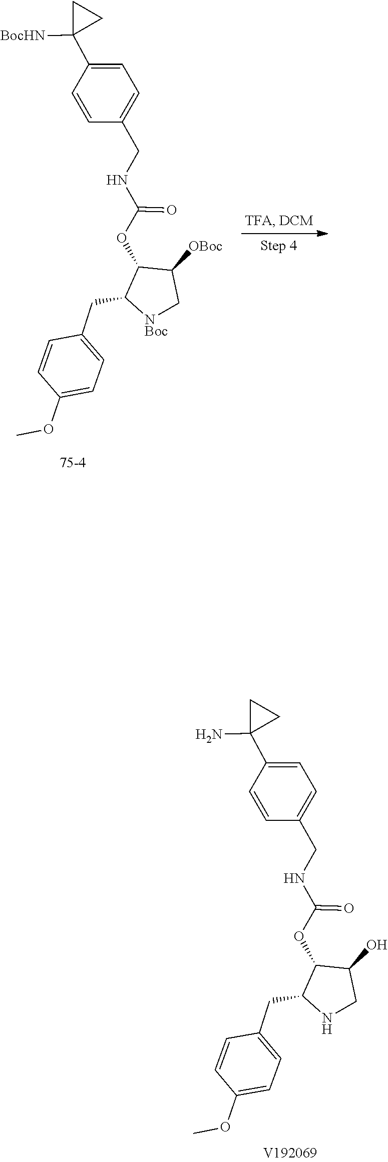

Step 1: To a stirred mixture of 4-(1-aminocyclopropyl)benzonitrile (200 mg, 1.26 mmol, 1 equiv) and di-tert-butyl dicarbonate (827.7 mg, 3.79 mmol, 3 equiv) in Water (5 mL) and methanol (5 mL) was added NaHCO₃ (318.6 mg, 3.79 mmol, 3 equiv) at room temperature. The resulting mixture was stirred at room temperature for overnight. The reaction mixture was quenched with water and extracted with EA. The extracts were washed with brine, dried over anhydrous sodium sulfate, filtered and concentrated. The residue was purified by reversed-phase flash chromatography to afford tert-butyl N-[1-(4-cyanophenyl)cyclopropyl]carbamate (200 mg, 61.2% yield, 90% purity) as a white solid.

Step 2: To a stirred mixture of tert-butyl N-[1-(4-cyanophenyl)cyclopropyl]carbamate (341 mg, 1.32 mmol, 1 equiv) in methanol (5 mL) was added raney nickel (77.4 mg, 1.32 mmol, 1 equiv) and Ammonium hydroxide (9% in water) (13.8 mg, 0.39 mmol, 0.3 equiv) at 0° C. under nitrogen atmosphere. The resulting mixture was stirred at room temperature for overnight under hydrogen atmosphere. The resulting mixture was filtered, the filter cake was washed with methanol (1×2 mL). The filtrate was concentrated under reduced pressure. This resulted in tert-butyl N-{1-[4-(aminomethyl)phenyl]cyclopropyl}carbamate (300 mg, 86.6% yield, 90% purity) as a yellow oil. MS: m/z: Calc'd for C₁₅H₂₂N₂O₂ [M*2+H]+ 525. found 525.

Step 3 and 4: The title compound was prepared in 41.3% overall yield as a white solid according to Substitution reaction for synthesis of Carbamate; General Procedure I using tert-butyl N-{1-[4-(aminomethyl)phenyl]cyclopropyl}carbamate (75-2) in STEP 3; Boc Deprotection; General Procedure II in STEP 4. MS: m/z Calc'd for C₂₃H₂₉N₃O₄ [M+H]⁺412. found 412. ¹H NMR (400 MHz, Methanol-d₄) δ 7.52-7.45 (m, 2H), 7.42 (d, J=8.3 Hz, 2H), 7.26-7.16 (m, 2H), 6.92-6.85 (m, 2H), 4.94 (d, J=3.5 Hz, 1H), 4.44-4.29 (m, 3H), 4.20-4.11 (m, 1H), 3.79 (s, 3H), 3.64-3.55 (m, 1H), 3.22 (d, J=12.7 Hz, 1H), 3.09 (dd, J=14.1, 7.5 Hz, 1H), 2.98 (dd, J=14.3, 8.4 Hz, 1H), 1.43-1.34 (m, 2H), 1.33-1.25 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 27% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-{[3-(imidazol-1-yl)phenyl]methyl}carbamate (V192167)

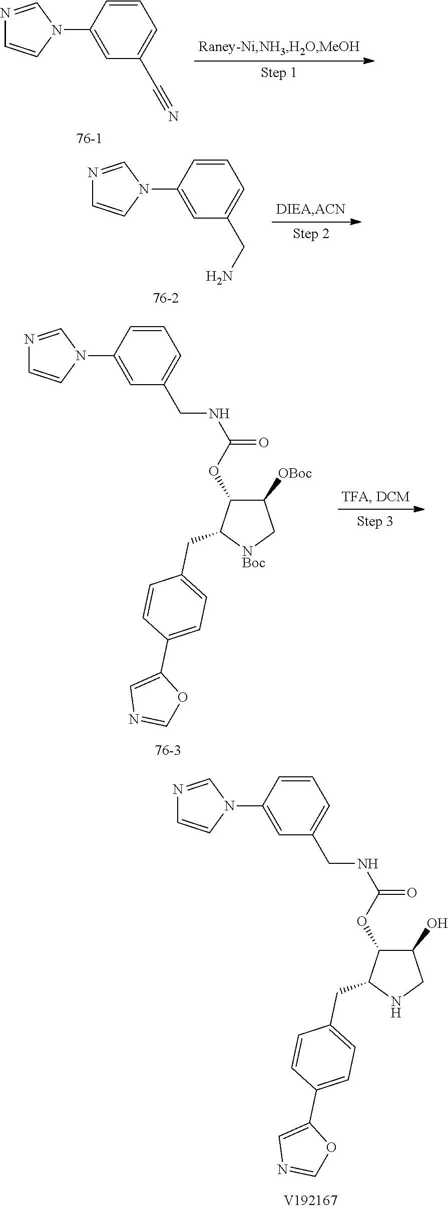

Step 1: To a stirred mixture of 3-(imidazol-1-yl)benzonitrile (300 mg, 1.77 mmol, 1 equiv) and Ammonium hydroxide (9% in water) (62.1 mg, 1.77 mmol, 1 equiv) in methanol (10 mL) was added raney nickel (1040.7 mg, 17.73 mmol, 10 equiv) at room temperature under nitrogen atmosphere. The resulting mixture was stirred at room temperature for overnight under hydrogen atmosphere. The resulting mixture was filtered. the filter cake was washed with MeOH (1×2 mL). The filtrate was concentrated under reduced pressure. This resulted in 1-[3-(imidazol-1-yl)phenyl]methanamine (200 mg, 65.1% yield, 90% purity) as a yellow solid. MS: m/z: Calc'd for C₁₀H₁₁N₃ [M+H]+ 174. found 174.

Step 2 and 3: The title compound was prepared in 33.1% overall yield as a yellow solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-[3-(imidazol-1-yl)phenyl]methanamine (76-2) in STEP 2; Boc Deprotection; General Procedure II in STEP 3. MS: m/z Calc'd for C₂₅H₂₅N₅O₄ [M+H]⁺460. found 460. ¹H NMR (400 MHz, Methanol-d₄) δ 9.34 (s, 1H), 8.26 (s, 1H), 8.03 (s, 1H), 7.79-7.57 (m, 7H), 7.51 (s, 1H), 7.40 (d, J=8.0 Hz, 2H), 5.00 (d, J=3.5 Hz, 1H), 4.64-4.40 (m, 3H), 4.31-4.22 (m, 1H), 3.62 (dd, J=12.7, 4.3 Hz, 1H), 3.29-3.17 (m, 2H), 3.11 (dd, J=14.1, 8.3 Hz, 1H).

Prep-HPLC purification conditions: Column: Xselect CSH Prep Fluoro-Phenyl Column, 30*150 mm, 5 μm; Mobile Phase A: Water (0.05% TFA), Mobile Phase B: ACN; Flow rate: 60 mL/min; Gradient: 2% B to 19% B in 8 min; Wave Length: 254 nm/220 nm; RT1(min): 6.65.

### Synthetic Procedure of (2R,3S,4S)-4-hydroxy-2-{[4-(pyridin-3-yloxy)phenyl]methyl}pyrrolidin-3-yl N-[(3-fluorophenyl)methyl]carbamate (V192398)

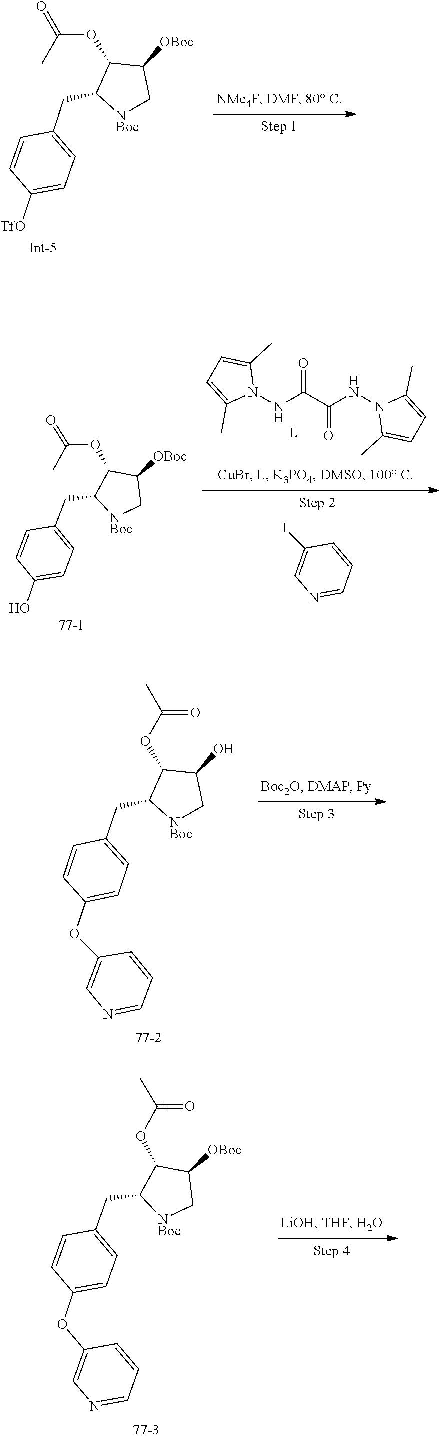

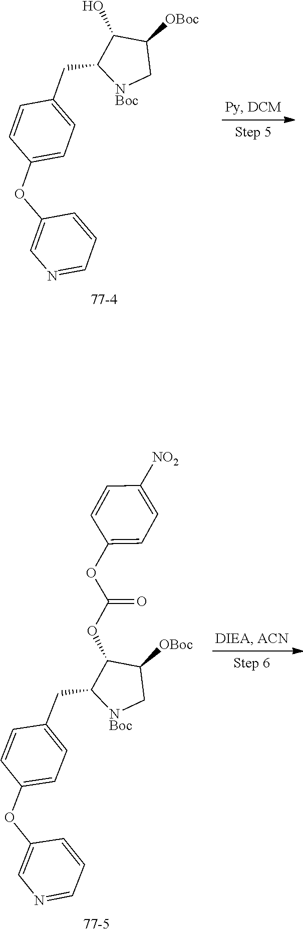

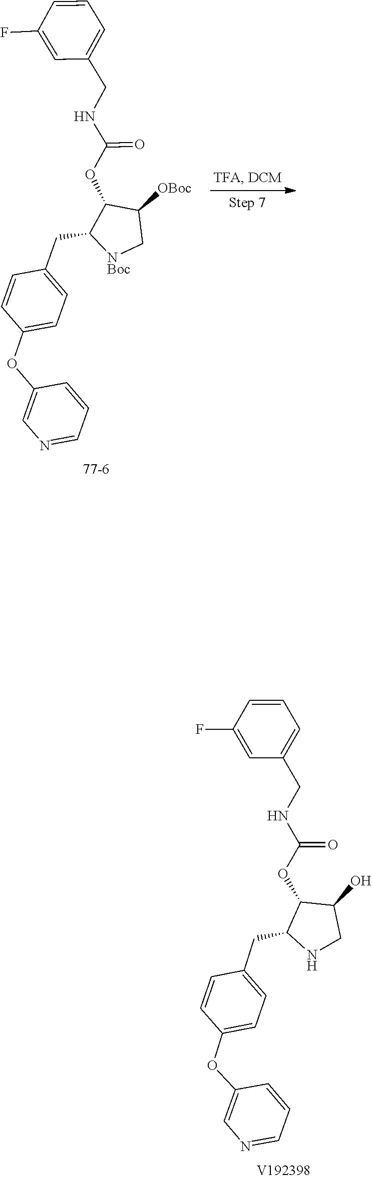

Step 1: To the solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (500 mg, 0.86 mmol, 1 equiv) in DMF (5 mL) was added Tetramethylammonium fluoride (319 mg, 3.43 mmol, 4 equiv). The mixture was stirred at 80° C. for 5 hours under N₂. The mixture was purified by reversed-phase flash chromatography to afford tert-butyl (2R,3S,4S)-3-(acetyloxy)-4-[(tert-butoxycarbonyl)oxy]-2-[(4-hydroxyphenyl)methyl]pyrrolidine-1-carboxylate (250 mg, 64.6% yield) was obtained as a white solid. MS: m/z: Calc'd for C₂₃H₃₃NO₈ [M+H]⁺452. found 452.

Step 2: To the solution of tert-butyl (2R,3S,4S)-3-(acetyloxy)-4-[(tert-butoxycarbonyl)oxy]-2-[(4-hydroxyphenyl)methyl]pyrrolidine-1-carboxylate (200 mg, 0.44 mmol, 1 equiv), 3-iodopyridine (136 mg, 0.66 mmol, 1.5 equiv) and N,N′-bis(2,5-dimethyl-1H-pyrrol-1-yl)ethanediamide (24 mg, 0.089 mmol, 0.2 equiv) in DMSO (5 mL) was added CuBr (6 mg, 0.044 mmol, 0.1 equiv) and K₃PO₄ (188 mg, 0.89 mmol, 2 equiv). The mixture was stirred at 100° C. for 2 days under N₂. The mixture was diluted with EA, washed with water, dried and concentrated. The crude product was used in the next step without purification as a brown oil. MS: m/z: Calc'd for C₂₃H₂₈N₂O₆ [M+H]⁺429. found 429.

Step 3: To the solution of tert-butyl (2R,3S,4S)-3-(acetyloxy)-4-hydroxy-2-{[4-(pyridin-3-yloxy)phenyl]methyl}pyrrolidine-1-carboxylate (80 mg, 0.19 mmol, 1 equiv) and di-tert-butyl dicarbonate (204 mg, 0.94 mmol, 5 equiv) in pyridine (5 mL) was added DMAP (34 mg, 0.28 mmol, 1.5 equiv). The mixture was stirred at rt for overnight. The mixture was concentrated. The residue was purified by Prep TLC (PE:EA=1:1) to afford tert-butyl (2R,3S,4S)-3-(acetyloxy)-4-[(tert-butoxycarbonyl)oxy]-2-{[4-(pyridin-3-yloxy)phenyl]methyl}pyrrolidine-1-carboxylate (60 mg, 60.8% yield) as a colorless oil. MS: m/z: Calc'd for C₂₈H₂₆N₂O₈ [M+H]⁺529. found 529.

Step 4: To the solution of tert-butyl (2R,3S,4S)-3-(acetyloxy)-4-[(tert-butoxycarbonyl)oxy]-2-{[4-(pyridin-3-yloxy)phenyl]methyl}pyrrolidine-1-carboxylate (60 mg, 0.11 mmol, 1 equiv) in THF (3 mL) and H₂O (1 mL) was added LiOH (16 mg, 0.684 mmol, 6 equiv). The mixture was stirred at rt for 2 hours. The mixture was diluted with EA, washed with water, dried and concentrated. The crude product was used directly in the next step without further purification. MS: m/z: Calc'd for C₂₆H₃₄N₂O₇ [M+H]⁺487. found 487.

Step 5: To the solution of tert-butyl (2R,3S,4S)-4-[(tert-butoxycarbonyl)oxy]-3-hydroxy-2-{[4-(pyridin-3-yloxy)phenyl]methyl}pyrrolidine-1-carboxylate (30 mg, 0.062 mmol, 1 equiv) and 4-nitrophenyl carbonochloridate (25 mg, 0.12 mmol, 2 equiv) in DCM (3 mL) was added pyridine (10 mg, 0.12 mmol, 2 equiv). The mixture was stirred at rt for 2 hours. The mixture was concentrated. The residue was purified by reversed-phase flash chromatography to afford tert-butyl (2R,3S,4S)-4-[(tert-butoxycarbonyl)oxy]-3-[(4-nitrophenoxycarbonyl)oxy]-2-{[4-(pyridin-3-yloxy)phenyl]methyl}pyrrolidine-1-carboxylate (20 mg, 49.8% yield) as a colorless oil. MS: m/z: Calc'd for C₃₃H₃₇N₃O₁₁ [M+H]⁺652. found 652.

### Step 6 and 7

The title compound was prepared in 27.3% overall yield as a grey solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(pyridin-3-yloxy)phenyl]methyl}pyrrolidine-1-carboxylate (77-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 6; Boc Deprotection; General Procedure II in STEP 7. MS: m/z Calc'd for C₂₄H₂₄F₂N₃O₄[M+H]⁺438. found 438. ¹H NMR (400 MHz, Methanol-d₄) δ 8.41 (s, 2H), 7.64 (s, 2H), 7.41-7.29 (m, 3H), 7.15 (d, J=7.6 Hz, 1H), 7.12-7.04 (m, 3H), 7.04-6.94 (m, 1H), 4.97 (d, J=3.6 Hz, 1H), 4.44-4.34 (m, 2H), 4.34-4.21 (m, 2H), 3.62 (dd, J=12.5, 4.1 Hz, 1H), 3.28 (d, J=12.7 Hz, 1H), 3.24-3.06 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 4% B to 34% B in 10 min; Wave Length: 254 nm/220 nm; RT1(min): 8.5.

### Synthetic Procedure of (2R,3S,4S)-2-({4-[2-(3,3-difluoroazetidin-1-yl)-1,3-thiazol-5-yl]phenyl}methyl)-4-hydroxypyrrolidin-3-yl N-[(3-fluorophenyl)methyl]carbamate (V192410)

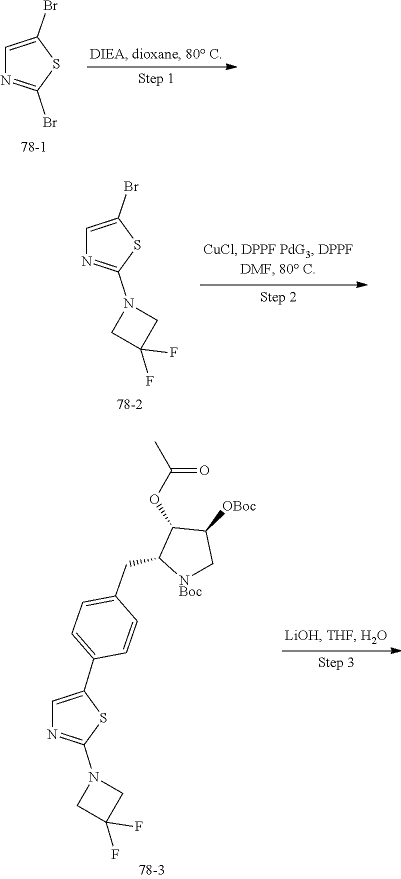

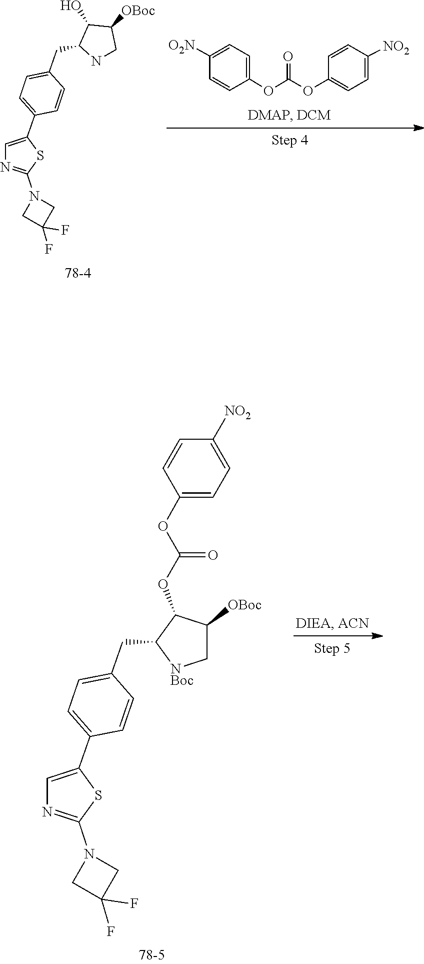

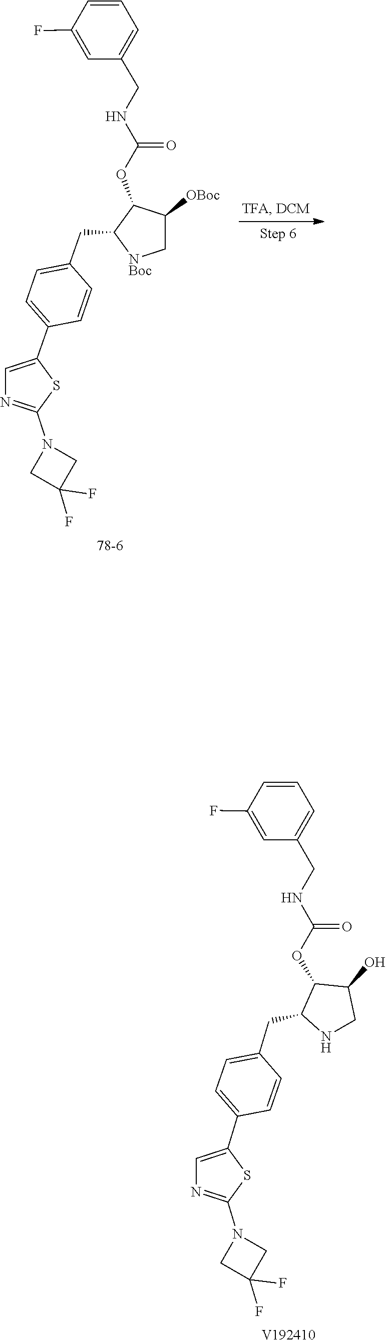

Step 1: To the solution of 2,5-dibromo-1,3-thiazole (500 mg, 2.06 mmol, 1 equiv) and 3,3-difluoroazetidine (287 mg, 3.09 mmol, 1.5 equiv) in DMF (3 mL) was added K₂CO₃ (532 mg, 4.12 mmol, 2 equiv). The mixture was stirred at 80° C. for overnight. The mixture was filtered and the filtrate was purified by reversed-phase flash chromatography to afford 5-bromo-2-(3,3-difluoroazetidin-1-yl)-1,3-thiazole (300 mg, 57.1% yield) as a light yellow solid. MS: m/z: Calc'd for C₆H₅BrF₂N₂S[M+H]⁻255. found 255.

Step 2: To the solution of tert-butyl (2R,3S,4S)-3-(acetyloxy)-4-[(tert-butoxycarbonyl)oxy]-2-{[4-(tributylstannyl)phenyl]methyl}pyrrolidine-1-carboxylate (300 mg, 0.41 mmol, 1 equiv), dppf (46 mg, 0.083 mmol, 0.2 equiv) and 5-bromo-2-(3,3-difluoroazetidin-1-yl)-1,3-thiazole (158 mg, 0.62 mmol, 1.5 equiv) in DMF (5 mL) was added CuCl (82 mg, 0.828 mmol, 2 equiv) and Dppf palladium(II) biphenyl-2-amine (38 mg, 0.041 mmol, 0.1 equiv). The mixture was stirred at 80° C. for overnight under N₂. The mixture was purified by reversed-phase flash chromatography to afford tert-butyl (2R,3S,4S)-3-(acetyloxy)-4-[(tert-butoxycarbonyl)oxy]-2-({4-[2-(3,3-difluoroazetidin-1-yl)-1,3-thiazol-5-yl]phenyl}methyl)pyrrolidine-1-carboxylate (66 mg, 26.15% yield) as a brown oil. MS: m/z: Calc'd for C₂₉H₃₇F₂N₃O₇S [M+H]⁺610. found 610.

Step 3: To the solution of tert-butyl (2R,3S,4S)-3-(acetyloxy)-4-[(tert-butoxycarbonyl)oxy]-2-({4-[2-(3,3-difluoroazetidin-1-yl)-1,3-thiazol-5-yl]phenyl}methyl)pyrrolidine-1-carboxylate (66 mg, 0.11 mmol, 1 equiv) in THF (5 mL) and H₂O (1 mL) was added LiOH (16 mg, 0.65 mmol, 6 equiv). The mixture was stirred at rt for 4 hours. The mixture was diluted with EA, washed with water, dried and concentrated. The crude product was used in the next step without further purification. MS: m/z: Calc'd for C₂₇H₃₅F₂N₃O₆S [M+H]⁺568. found 568.

Step 4: To the solution of tert-butyl (2R,3S,4S)-4-[(tert-butoxycarbonyl)oxy]-2-({4-[2-(3,3-difluoroazetidin-1-yl)-1,3-thiazol-5-yl]phenyl}methyl)-3-hydroxypyrrolidine-1-carboxylate (40 mg, 0.070 mmol, 1 equiv) and bis(4-nitrophenyl) carbonate (43 mg, 0.14 mmol, 2 equiv) in DCM (3 mL) was added DMAP (17 mg, 0.14 mmol, 2 equiv). The mixture was stirred at rt for 6 hours. The mixture was diluted with DCM, washed with water, dried and concentrated. The crude product was used directly in the next step without further purification. MS: m/z: Calc'd for C₃₄H₃₈F₂N₄O₁₀S [M+H]⁺733, found 733.

### Step 5 and 6

The title compound was prepared in 33.2% overall yield as a white solid according to Substitution reaction for synthesis of Carbamate; General Procedure I using tert-butyl (2R,3S,4S)-4-[(tert-butoxycarbonyl)oxy]-2-({4-[2-(3,3-difluoroazetidin-1-yl)-1,3-thiazol-5-yl]phenyl}methyl)-3-[(4-nitrophenoxycarbonyl)oxy]pyrrolidine-1-carboxylate (76-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 5; Boc Deprotection; General Procedure II in STEP 6. MS: m/z Calc'd for C₂₅H₂₅F₃N₄O₃S [M+H]⁺519. found 519. ¹H NMR (400 MHz, Methanol-d₄) δ 7.51 (s, 1H), 7.49-7.43 (m, 2H), 7.43-7.34 (m, 1H), 7.31 (d, J=8.1 Hz, 2H), 7.16 (d, J=7.6 Hz, 1H), 7.10 (d, J=10.1 Hz, 1H), 7.08-7.00 (m, 1H), 4.95 (d, J=3.1 Hz, 1H), 4.57-4.47 (m, 4H), 4.47-4.36 (m, 3H), 4.31 (d, J=15.4 Hz, 1H), 4.27-4.19 (m, 1H), 3.60 (dd, J=12.6, 4.2 Hz, 1H), 3.25 (d, J=12.6 Hz, 1H), 3.22-3.13 (m, 1H), 3.11-3.01 (m, 1H).

Prep-HPLC purification conditions: Column: Xselect CSH Prep Fluoro-Phenyl Column, 30*150 mm, 5 μm; Mobile Phase A: Water (0.05% TFA), Mobile Phase B: ACN; Flow rate: 60 mL/min; Gradient: 11% B to 41% B in 7 min; Wave Length: 254 nm/220 nm; RT1(min): 6.78.

### Synthetic Procedure of (2R,3S,4S)-4-hydroxy-2-{[4-(1,3-oxazol-5-yl)phenyl]methyl}pyrrolidin-3-yl N-[2-(3,5-difluoro-4-hydroxyphenyl)ethyl]carbamate (V192432)

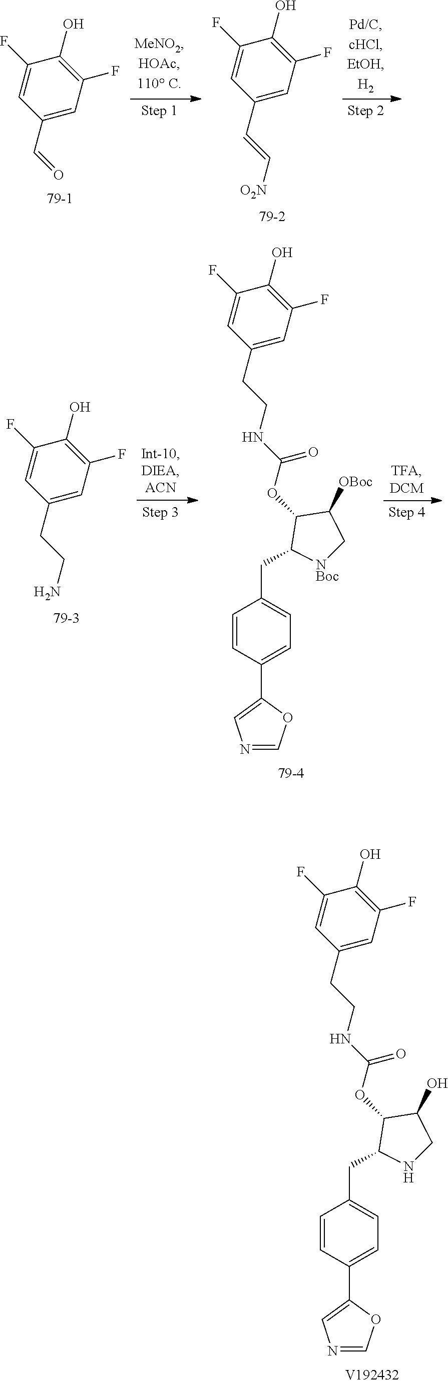

Step 1: To the solution of 3,5-difluoro-4-hydroxybenzaldehyde (200 mg, 1.26 mmol, 1 equiv) and nitromethane (463 mg, 7.59 mmol, 6 equiv) in AcOH (4 mL) was added ammonium acetate (481 mg, 5.06 mmol, 4 equiv). The mixture was stirred at 110° C. for 2 hours under N₂. The mixture was concentrated and the crude product was purified by Prep TLC (PE:EA=1:1). The 2,6-difluoro-4-[(E)-2-nitroethenyl]phenol (120 mg, 47.2% yield) was obtained as a brown solid. MS: m/z: Calc'd for C₈H₅F₂NO₃[M−H]⁻200. found 200.

Step 2: To the solution of 2,6-difluoro-4-[(E)-2-nitroethenyl]phenol (110 mg, 0.55 mmol, 1 equiv) in EtOH (5 mL) was Pd/C (25 mg, 0.24 mmol, 0.43 equiv) and 1 drop HCl (12 M in H₂O). The mixture was stirred at rt for overnight under H₂. The mixture was filtered and the filtrate was concentrated to give the crude product used directly in the next step. The 4-(2-aminoethyl)-2,6-difluorophenol (80 mg, 84.5% yield) was obtained as a yellow oil. MS: m/z: Calc'd for C₈H₉F₂NO [M+H]⁺174. found 174.

Step 3 and 4: The title compound was prepared in 33.1% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 4-(2-aminoethyl)-2,6-difluorophenol (76-2) in STEP 3; Boc Deprotection; General Procedure II in STEP 4. MS: m/z Calc'd for C₂₃H₂₃F₂N₃O₅ [M+H]⁺460. found 460. ¹H NMR (400 MHz, Methanol-d₄) δ 8.27 (s, 1H), 7.77-7.70 (m, 2H), 7.54 (s, 1H), 7.39 (d, J=8.2 Hz, 2H), 6.90-6.77 (m, 2H), 4.96-4.90 (m, 1H), 4.34 (d, J=4.2 Hz, 1H), 4.26-4.17 (m, 1H), 3.55 (dd, J=12.6, 4.3 Hz, 1H), 3.50-3.34 (m, 2H), 3.23 (d, J=12.6 Hz, 1H), 3.21-3.11 (m, 1H), 3.08-2.98 (m, 1H), 2.84-2.68 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 20% B to 35% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-[(3,5-difluoro-4-hydroxyphenyl)methyl]carbamate (V192433)

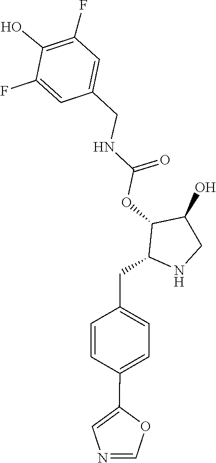 [SMILES: O=C(NCc1cc(F)c(O)c(F)c1)O[C@@H]1[C@@H](O)CN[C@@H]1Cc1ccc(-c2cnco2)cc1]

The title compound was prepared in 25.8% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 4-(aminomethyl)-2,6-difluorophenol in STEP 1; Boc Deprotection; General Procedure II in STEP 2. MS: m/z Calc'd for C₂₂H₂₁F₂N₃O₅[M+H]⁺446. found 446. ¹H NMR (400 MHz, Methanol-d₄) δ 8.28 (s, 1H), 7.76-7.68 (m, 2H), 7.54 (s, 1H), 7.40 (d, J=8.1 Hz, 2H), 6.99-6.87 (m, 2H), 4.96 (d, J=3.4 Hz, 1H), 4.45-4.39 (m, 1H), 4.36-4.23 (m, 2H), 4.19 (d, J=15.2 Hz, 1H), 3.60 (dd, J=12.6, 4.2 Hz, 1H), 3.31-3.17 (m, 2H), 3.16-3.06 (m, 1H).

Prep-HPLC purification conditions: Column: SHIMADZU Shim-pack Scepter C18-120, 30*150 mm, 5 μm; Mobile Phase A: Water (0.05% TFA), Mobile Phase B: ACN; Flow rate: 60 mL/min; Gradient: 5% B to 35% B in 10 min; Wave Length: 254 nm/220 nm; RT1(min): 9.3.

### Synthetic Procedure of (2R,3S,4S)-4-hydroxy-2-{[4-(1,3-oxazol-5-yl)phenyl]methyl}pyrrolidin-3-yl N-{2-[(4R)-3,3-difluoropiperidin-4-yl]ethyl}carbamate (V192894)

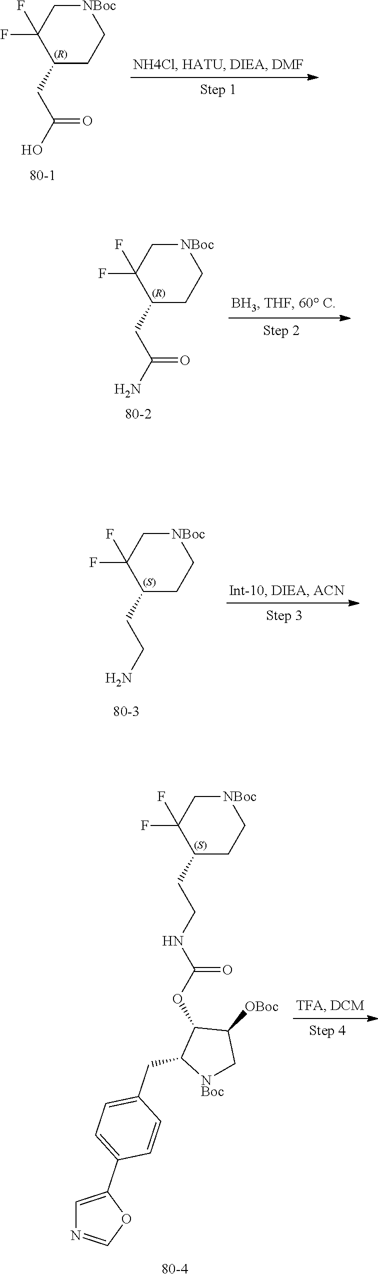

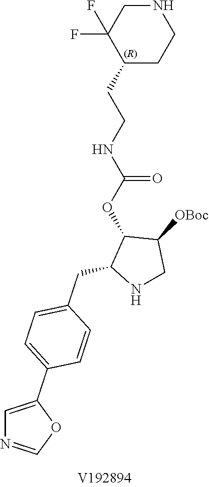

Step 1: To the solution of [(4R)-1-(tert-butoxycarbonyl)-3,3-difluoropiperidin-4-yl]acetic acid (200 mg, 0.716 mmol, 1 equiv) and HATU (326.75 mg, 0.859 mmol, 1.2 equiv) in DMF (3 mL) was added ammonium chloride (76.61 mg, 1.432 mmol, 2 equiv) and DIEA (185.11 mg, 1.432 mmol, 2 equiv). The mixture was stirred at rt for overnight. LCMS showed the reaction was OK. The mixture was diluted with EA, washed with water, dried and concentrated. The crude product was used directly in the next step without further purification. MS: m/z: Calc'd for C₁₂H₂₀F₂NO₃ [M+H]⁺279. found 279.

Step 2: To the solution of tert-butyl (4R)-4-(carbamoylmethyl)-3,3-difluoropiperidine-1-carboxylate (150 mg, 0.54 mmol, 1 equiv) in THF (5 mL) was added Borane-tetrahydrofuran complex (1.0M in THF) (463.21 mg, 5.390 mmol, 10 equiv). The mixture was stirred at 60° C. for 2 hours. The mixture was concentrated to give the crude product which was used in the next step without further purification, as a colorless oil. MS: m/z: Calc'd for C₁₂H₂₂F₂N₂O₂[M+H]⁺265. found 265.

Step 3 and 4: The title compound was prepared in 17.5% overall yield as an off-white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (4S)-4-(2-aminoethyl)-3,3-difluoropiperidine-1-carboxylate (80-3) in STEP 3; Boc Deprotection; General Procedure II in STEP 4. MS: m/z Calc'd for C₂₂H₂₈F₂N₄O₄ [M+H]⁺451. found 451. ¹H NMR (400 MHz, Methanol-d₄) δ 8.27 (s, 1H), 7.75 (d, J=7.9 Hz, 2H), 7.54 (s, 1H), 7.45 (d, J=7.9 Hz, 2H), 4.98 (d, J=3.6 Hz, 1H), 4.40 (d, J=4.1 Hz, 1H), 4.30-4.21 (m, 1H), 3.78-3.66 (m, 1H), 3.60 (dd, J=12.6, 4.3 Hz, 1H), 3.50-3.35 (m, 3H), 3.32-3.18 (m, 3H), 3.11-3.09 (m, 2H), 2.24 (d, J=15.7 Hz, 2H), 2.13-2.01 (m, 1H), 1.81-1.70 (m, 1H), 1.64-1.54 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-{2-[(3S)-azepan-3-yl]ethyl}carbamate (V193013)

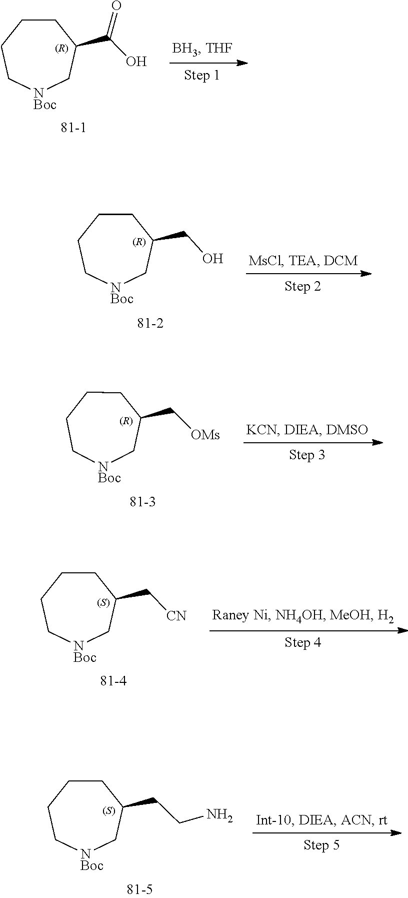

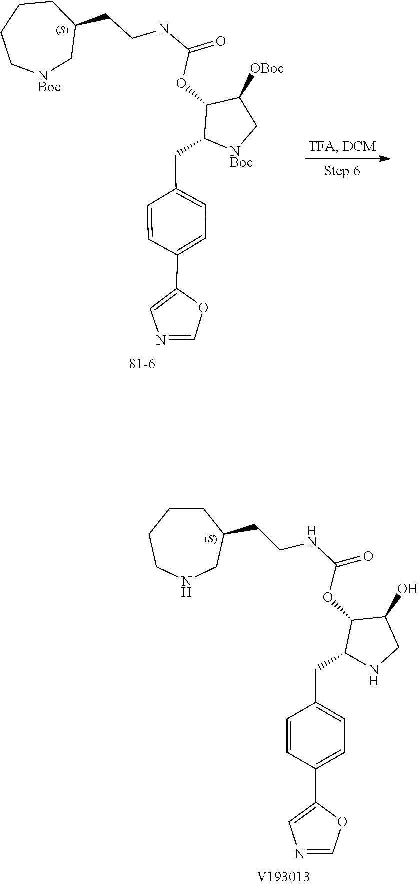

Step 1: To a stirred mixture of (3R)-1-(tert-butoxycarbonyl)azepane-3-carboxylic acid (500 mg, 2.06 mmol, 1 equiv) in THF (50 mL) was added Borane-tetrahydrofuran complex (1.0M in THF) (883 mg, 10.28 mmol, 5 equiv) at 0° C. under nitrogen atmosphere. The resulting mixture was stirred at room temperature for overnight under nitrogen atmosphere. The reaction was quenched with MeOH at 0° C. The resulting mixture was concentrated under reduced pressure to afford crude product which was used directly in the next step without further purification as a yellow oil. MS: m/z: Calc'd for C₁₂H₂₃NO₃ [M+H]⁺230. found 230.

Step 2: To the solution of tert-butyl (3R)-3-(hydroxymethyl)azepane-1-carboxylate (460 mg, 2.01 mmol, 1 equiv) in DCM (5 mL) was added methanesulfonyl chloride (345 mg, 3.01 mmol, 1.5 equiv) and Et₃N (406 mg, 4.01 mmol, 2 equiv). The mixture was stirred at rt for 1 hour. The mixture was diluted with DCM, washed with water, dried and concentrated. The crude product was used in the next step without further purification. The tert-butyl (3R)-3-[(methanesulfonyloxy)methyl]azepane-1-carboxylate (450 mg, 73.0% yield) was obtained as a light yellow oil. MS: m/z: Calc'd for C₁₃H₂₅NO₅S[M+H−56]⁺252. found 252.

Step 3: To the solution of tert-butyl (3R)-3-[(methanesulfonyloxy)methyl]azepane-1-carboxylate (450 mg, 1.46 mmol, 1 equiv) and KCN (191 mg, 2.93 mmol, 2 equiv) in DMSO (5 mL) was added DIEA (378 mg, 2.93 mmol, 2 equiv). The mixture was stirred at 80° C. for overnight. The mixture was diluted with EA, washed with water, dried and concentrated. The crude product was used in the next step without further purification as a yellow oil. MS: m/z: Calc'd for C₁₃H₂₂N₂O₂ [M+H−56]⁺183. found 183.

Step 4: To the solution of tert-butyl (3S)-3-(cyanomethyl)azepane-1-carboxylate (100 mg, 0.42 mmol, 1 equiv) in methanol (5 mL) was added raney nickel (30 mg, 0.51 mmol, 1.22 equiv) and 5 drops NH₄OH. The mixture was stirred at rt for 4 hours under H₂. The mixture was filtered and concentrated to give the crude product. The tert-butyl (3S)-3-(2-aminoethyl)azepane-1-carboxylate (85 mg, 83.6% yield) was obtained as a colorless oil. MS: m/z: Calc'd for C₁₃H₂₆N₂O₂ [M+H]⁺243. found 243.

### Step 5 and 6

The title compound was prepared in 52.2% overall yield as an off-white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (3S)-3-(2-aminoethyl)azepane-1-carboxylate (81-5) in STEP 5; Boc Deprotection; General Procedure II in STEP 6. MS: m/z Calc'd for C₂₃H₃₂N₄O₄[M+H]⁺429. found 429. ¹H NMR (400 MHz, Methanol-d₄) δ 8.27 (s, 1H), 7.79-7.72 (m, 2H), 7.54 (s, 1H), 7.48-7.41 (m, 2H), 5.02-4.96 (m, 1H), 4.39 (d, J=4.1 Hz, 1H), 4.30-4.21 (m, 1H), 3.60 (dd, J=12.7, 4.3 Hz, 1H), 3.37-3.33 (m, 1H), 3.29-3.15 (m, 6H), 3.10 (dd, J=14.2, 8.6 Hz, 1H), 2.95 (dd, J=13.5, 10.1 Hz, 1H), 1.99-1.88 (m, 5H), 1.66-1.53 (m, 3H), 1.49-1.36 (m, 1H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.5.

### Synthetic Procedure of (2R,3S,4S)-2-[(4-azidophenyl)methyl]-4-hydroxypyrrolidin-3-yl N-[(3-fluorophenyl)methyl]carbamate (V193037)

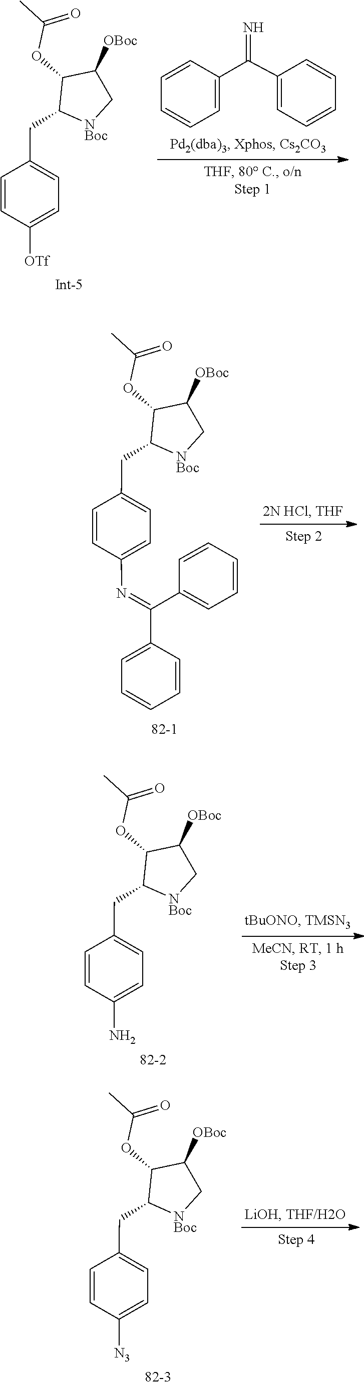

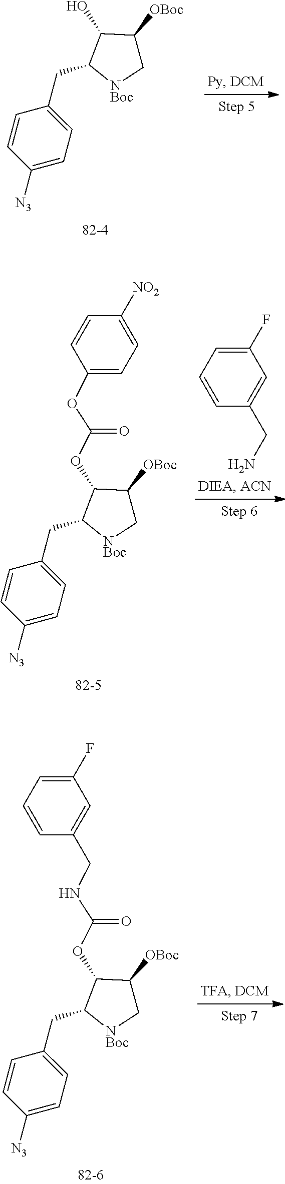

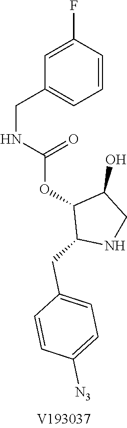 [SMILES: [N-]=[N+]=Nc1ccc(C[C@H]2NC[C@H](O)[C@H]2OC(=O)NCc2cccc(F)c2)cc1]

Step 1: A solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (1 g, 1.71 mmol, 1 equiv) in THF (10 mL) was treated with Pd₂(dba)₃ (0.16 g, 0.17 mmol, 0.1 equiv) and Cs₂CO₃ (1.67 g, 5.14 mmol, 3 equiv) at room temperature. the mixture replacing nitrogen gas for 5 min. The final reaction mixture was stirred at 80° C. for 12h. Desired product could be detected by LCMS. The resulting mixture was filtered, the filter cake was washed with EA (50 mL). The filtrate was concentrated under reduced pressure. The residue was purified by reversed-phase flash chromatography with the following conditions: column, C18 silica gel; mobile phase, 0.05% NH₄HCO₃ in MeCN, 0% to 100% gradient in 30 min; detector, UV 254 nm). to afford tert-butyl (2R,3S,4S)-3-(acetyloxy)-4-[(tert-butoxycarbonyl)oxy]-2-({4-[(diphenylmethylidene)amino]phenyl}methyl)pyrrolidine-1-carboxylate (220 mg, 17% yield) as a yellow oil. MS: m/z: Calc'd for C₃₆H₄₂N₂O₇ [M+H]⁺615. found 615.

Step 2: A solution of tert-butyl (2R,3S,4S)-3-(acetyloxy)-4-[(tert-butoxycarbonyl)oxy]-2-({4-[(diphenylmethylidene)amino]phenyl}methyl)pyrrolidine-1-carboxylate (220 mg, 0.35 mmol, 1 equiv) in THF (2.5 mL) was treated with 2N HCl (0.5 mL, 2 mmol) at 0° C. for 5 min under nitrogen atmosphere. The resulting mixture was stirred at 25° C. for additional 4h. Desired product could be detected by LCMS. The resulting mixture was concentrated under reduced pressure. The residue was purified by Prep-TLC (EA:PE 1:1) to afford tert-butyl (2R,3S,4S)-3-(acetyloxy)-2-[(4-aminophenyl)methyl]-4-[(tert-butoxycarbonyl)oxy]pyrrolidine-1-carboxylate (130 mg, 80% yield, 95% purity) as a yellow oil. MS: m/z: Calc'd for C₂₃H₃₄N₂O₇ [M+H]⁺451. found 451.

Step 3: A solution of tert-butyl (2R,3S,4S)-3-(acetyloxy)-2-[(4-aminophenyl)methyl]-4-[(tert-butoxycarbonyl)oxy]pyrrolidine-1-carboxylate (130 mg, 0.28 mmol, 1 equiv) in MeCN (5 mL) was treated with tert-butylnitrite (44 mg, 0.43 mmol, 1.5 equiv) at 0° C. for 5 min under nitrogen atmosphere followed by the addition of azidotrimethylsilane (49 mg, 0.43 mmol, 1.5 equiv) dropwise at 0° C. The resulting mixture was stirred at 25° C. for 1 h. Desired product could be detected by LCMS. The resulting mixture was concentrated under vacuum. The residue was purified by reversed-phase flash chromatography to afford tert-butyl (2R,3S,4S)-3-(acetyloxy)-2-[(4-azidophenyl)methyl]-4-[(tert-butoxycarbonyl)oxy]pyrrolidine-1-carboxylate (160 mg, 116% yield, 99.8% purity) as a yellow oil. MS: m/z: Calc'd for C₂₃H₃₂N₄O₇ [M+H]⁺477. found 477.

Step 4: A solution of tert-butyl (2R,3S,4S)-3-(acetyloxy)-2-[(4-azidophenyl)methyl]-4-[(tert-butoxycarbonyl)oxy]pyrrolidine-1-carboxylate (160 mg, 0.33 mmol, 1 equiv) in THF (5 mL) at room temperature followed by the addition of LiOH·H₂O (42 mg, 1.00 mmol, 3 equiv) in H₂O (1 mL) dropwise at 0° C. The resulting mixture was stirred at room temperature for 4h. Desired product could be detected by LCMS. The aqueous layer was extracted with EA (3×10 mL). The resulting mixture was concentrated under vacuum. The crude product was used in the next step directly without further purification. MS: m/z: Calc'd for C₂₁H₃₀N₄O₆ [M+H]⁺435. found 435.

Step 5: A solution of tert-butyl (2R,3S,4S)-2-[(4-azidophenyl)methyl]-4-[(tert-butoxycarbonyl)oxy]-3-hydroxypyrrolidine-1-carboxylate (150 mg, 0.34 mmol, 1 equiv) in DCM (5 mL) was treated with pyridine (81 mg, 1.03 mmol, 3 equiv) at 0° C. for 5 min under nitrogen atmosphere followed by the addition of 4-nitrophenyl carbonochloridate (104 mg, 0.51 mmol, 1.5 equiv) in DCM in portions at 0° C. The resulting mixture was stirred at 25° C. for 1 h. Desired product could be detected by LCMS. The resulting mixture was diluted with DCM (10 mL). The resulting mixture was concentrated under vacuum. The residue was purified by reversed-phase flash chromatography to afford tert-butyl (2R,3S,4S)-2-[(4-azidophenyl)methyl]-4-[(tert-butoxycarbonyl)oxy]-3-[(4-nitrophenoxycarbonyl)oxy]pyrrolidine-1-carboxylate (99 mg, 47% yield, 95% purity) as a white solid. MS: m/z: Calc'd for C₂₈H₃₃N₅O₁₀ [M+H]⁺600. found 600.

### Step 6 and 7

The title compound was prepared in 34.0% overall yield as an off-white solid according to Substitution reaction for synthesis of Carbamate; General Procedure I using tert-butyl (2R,3S,4S)-2-[(4-azidophenyl)methyl]-4-[(tert-butoxycarbonyl)oxy]-3-[(4-nitrophenoxycarbonyl)oxy]pyrrolidine-1-carboxylate (82-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 6; Boc Deprotection; General Procedure II in STEP 7. MS: m/z Calc'd for C₁₉H₂₀FN₅O₃[M+H]⁺386. found 386. ¹H NMR (400 MHz, Methanol-d₄) δ 7.39-7.35 (m, 1H), 7.34-7.29 (m, 2H), 7.16 (d, J=7.7 Hz, 1H), 7.12-6.99 (m, 4H), 4.44-4.35 (m, 2H), 4.30 (d, J=15.4 Hz, 1H), 4.22-4.16 (m, 1H), 3.59 (dd, J=12.5, 4.3 Hz, 1H), 3.25 (d, J=12.6 Hz, 1H), 3.16 (dd, J=14.1, 7.8 Hz, 1H), 3.05 (dd, J=14.0, 8.1 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.5.

### Synthetic Procedure of (2R,3S,4S)-2-[(4-azidophenyl)methyl]-4-hydroxypyrrolidin-3-yl N-{2-[(2S)-pyrrolidin-2-yl]ethyl}carbamate (V193038)

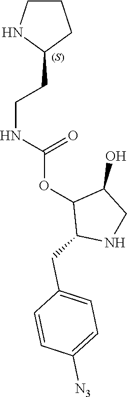

The title compound was prepared in 31.3% overall yield as off-white semi-solid according to Substitution reaction for synthesis of Carbamate; General Procedure I using tert-butyl (2R,3S,4S)-2-[(4-azidophenyl)methyl]-4-[(tert-butoxycarbonyl)oxy]-3-[(4-nitrophenoxycarbonyl)oxy]pyrrolidine-1-carboxylate (82-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate in STEP 1; Boc Deprotection; General Procedure II in STEP 2. MS: m/z Calc'd for C₁₈H₂₆N₆O₃ [M+H]⁺375. found 375. ¹H NMR (400 MHz, Methanol-d₄) δ 7.64-7.30 (m, 2H), 7.28-6.94 (m, 2H), 4.96 (s, 1H), 4.51-4.09 (m, 3H), 3.82-3.48 (m, 3H), 3.26-2.91 (m, 4H), 2.43-2.08 (m, 3H), 1.98-1.84 (m, 3H), 1.71-1.64 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.5.

### Synthetic Procedure of (2R,3S,4S)-4-hydroxy-2-[(4-nitrophenyl)methyl]pyrrolidin-3-yl N-{2-[(2S)-pyrrolidin-2-yl]ethyl}carbamate (V190542)

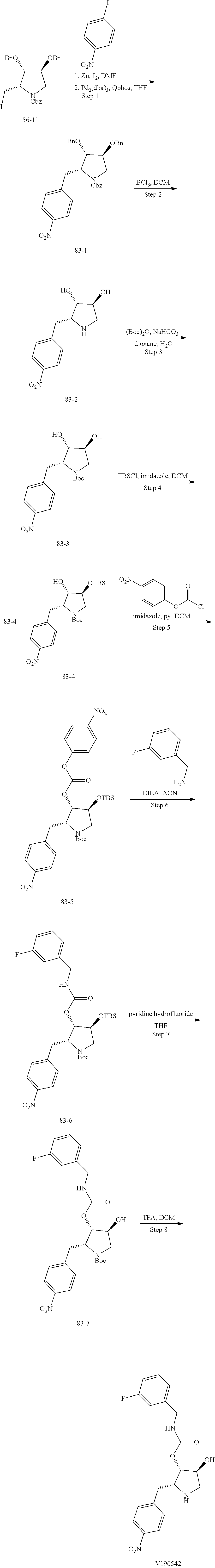

Step 1: A solution of benzyl (2S,3S,4S)-3,4-bis(benzyloxy)-2-(iodomethyl)pyrrolidine-1-carboxylate (1 g, 1.79 mmol, 1.0 eq.) in DMF (10 mL) was treated with Zinc (1.17 g, 17.94 mmol, 10.0 eq.) and Iodine (0.91 g, 3.59 mmol, 2.0 eq.) for 1 h at 25° C. under nitrogen atmosphere. Then a solution of benzene, 1-iodo-4-nitro- (0.89 g, 3.59 mmol, 2.0 eq.) in THF (10 mL) was treated with Pd₂(dba)₃ (0.16 g, 0.179 mmol, 0.1 equiv) and Q-Phos (0.26 g, 0.36 mmol, 0.2 eq.) under N₂. To the above mixture was added the first crude product dropwise 2 min at 25° C. The resulting mixture was stirred for additional 12 h at 25° C. under N₂. Desired product could be detected by LCMS. The residue was purified by reversed-phase flash chromatography to afford benzyl (2R,3S,4S)-3,4-bis(benzyloxy)-2-[(4-nitrophenyl)methyl]pyrrolidine-1-carboxylate (300 mg, 30.3% yield, 90% purity) as a light yellow oil. MS: m/z: Calc'd for C₃₃H₃₂N₂O₆ [M+H]⁺553. Found, 553.

Step 2: To a stirred solution of benzyl (2R,3S,4S)-3,4-bis(benzyloxy)-2-[(4-nitrophenyl)methyl]pyrrolidine-1-carboxylate (300 mg, 0.54 mmol, 1.0 eq.) in DCM (10 mL) was added trichloroborane (954.0 mg, 8.15 mmol, 15.0 eq.) in portions at 0° C. The resulting mixture was stirred over night at 25° C. The reaction was quenched with MeOH at 0° C. The crude product was used in the next step directly without further purification. MS: m/z: Calc'd for C₁₁H₁₄N₂O₄ [M+H]⁺239. Found, 239.

Step 3: To a stirred solution of (2R,3S,4S)-2-[(4-nitrophenyl)methyl]pyrrolidine-3,4-diol (170 mg, 0.71 mmol, 1.0 eq.) in 1,4-dioxane (6 mL) and H₂O (2 mL) was added di-tert-butyl dicarbonate (467.2 mg, 2.14 mmol, 3.0 eq.) and NaHCO₃ (179.8 mg, 2.14 mmol, 3.0 eq.) in portions at room temperature. The resulting mixture was stirred 2 h at 25° C. Desired product could be detected by LCMS. The residue was purified by reversed-phase flash chromatography to afford tert-butyl (2R,3S,4S)-3,4-dihydroxy-2-[(4-nitrophenyl)methyl]pyrrolidine-1-carboxylate (150 mg) as a light yellow oil. MS: m/z: Calc'd for C₁₆H₂₂N₂O₆ [M+H−56]⁺283. Found, 283.

Step 4: To a stirred solution of tert-butyl (2R,3S,4S)-3,4-dihydroxy-2-[(4-nitrophenyl)methyl]pyrrolidine-1-carboxylate (150 mg, 0.44 mmol, 1.0 eq.) in DCM (5 mL) was added tert-butyl(chloro)dimethylsilane (200.5 mg, 1.33 mmol, 3.0 eq.) and Imidazole (90.5 mg, 1.33 mmol, 3.0 eq.) in portions at 0° C. The resulting mixture was stirred 1 h at 25° C. Desired product could be detected by LCMS. The residue was purified by reversed-phase flash chromatography to afford tert-butyl (2R,3S,4S)-4-[(tert-butyldimethylsilyl)oxy]-3-hydroxy-2-[(4-nitrophenyl)methyl]pyrrolidine-1-carboxylate (160 mg) as a white solid. MS: m/z: Calc'd for C₂₂H₃₆N₂O₆Si [M+H−100]⁺353. Found, 353.

Step 5: To a stirred solution of tert-butyl (2R,3S,4S)-4-[(tert-butyldimethylsilyl)oxy]-3-hydroxy-2-[(4-nitrophenyl)methyl]pyrrolidine-1-carboxylate (160 mg, 0.35 mmol, 1.0 eq.) and 4-nitrophenyl carbonochloridate (142.5 mg, 0.706 mmol, 2.0 eq.) in DCM (5 mL) was added pyridine (55.9 mg, 0.71 mmol, 2.0 eq.) in portions at 0° C. The resulting mixture was stirred 1 h at 25° C. Desired product could be detected by LCMS. The residue was purified by reversed-phase flash chromatography to afford tert-butyl (2R,3S,4S)-4-[(tert-butyldimethylsilyl)oxy]-3-[(4-nitrophenoxycarbonyl)oxy]-2-[(4-nitrophenyl)methyl]pyrrolidine-1-carboxylate (130 mg) as a light yellow oil. MS: m/z: Calc'd for C₂₉H₃₉N₃O₁₀Si [M+H−100]⁺518. Found, 518.

### Step 6, 7, and 8

The title compound was prepared in 34.2% overall yield as a white solid according to Substitution reaction for synthesis of Carbamate; General Procedure I using tert-butyl (2R,3S,4S)-4-[(tert-butyldimethylsilyl)oxy]-3-[(4-nitrophenoxycarbonyl)oxy]-2-[(4-nitrophenyl)methyl]pyrrolidine-1-carboxylate (83-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 6; TBS Deprotection; General Procedure VII in STEP 7; Boc Deprotection; General Procedure II in STEP 8. MS: m/z Calc'd for C₁₉H₂₀FN₃O₅ [M+H]⁺390. Found, 390. ¹H NMR (400 MHz, Methanol-d₄) δ 8.25-8.17 (m, 2H), 7.56 (d, J=8.6 Hz, 2H), 7.39-7.36 (m, 1H), 7.16-7.00 (m, 3H), 4.99 (d, J=4.2 Hz, 1H), 4.46-4.27 (m, 4H), 3.61 (dd, J=12.6, 4.2 Hz, 1H), 3.33-3.16 (m, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 31% B in 10 min; Wave Length: 254 nm/220 nm; RT1(min): 8.5.

### Synthetic Procedure of: (2R,3S,4S)-4-hydroxy-2-[(4-nitrophenyl)methyl]pyrrolidin-3-yl N-{2-[(2S)-pyrrolidin-2-yl]ethyl}carbamate (V192359)

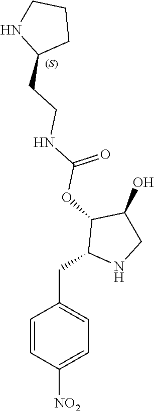

The title compound was prepared in 34.3% overall yield as a white solid according to Substitution reaction for synthesis of Carbamate; General Procedure I using tert-butyl (2R,3S,4S)-4-[(tert-butyldimethylsilyl)oxy]-3-[(4-nitrophenoxycarbonyl)oxy]-2-[(4-nitrophenyl)methyl]pyrrolidine-1-carboxylate (83-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate in STEP 1; Boc Deprotection; General Procedure II in STEP 2. MS: m/z Calc'd for C₁₈H₂₆N₄O₅ [M+H]⁺379. Found, 379. ¹H NMR (400 MHz, Methanol-d₄) δ 8.30-8.23 (m, 2H), 7.61 (d, J=8.6 Hz, 2H), 5.00 (d, J=3.6 Hz, 1H), 4.41 (d, J=4.0 Hz, 1H), 4.34 (s, 1H), 3.60-3.50 (m, 2H), 3.34-3.18 (m, 7H), 2.31 (dd, J=7.4, 3.9 Hz, 1H), 2.17-1.86 (m, 4H), 1.72 (dd, J=13.0, 8.9 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 31% B in 10 min; Wave Length: 254 nm/220 nm; RT1(min): 8.5.

### Synthetic Procedure of (2R,3S,4S)-4-hydroxy-2-{[4-(1,2-thiazol-5-yl)phenyl]methyl}pyrrolidin-3-yl N-{2-[(2S)-pyrrolidin-2-yl]ethyl}carbamate (V192331)

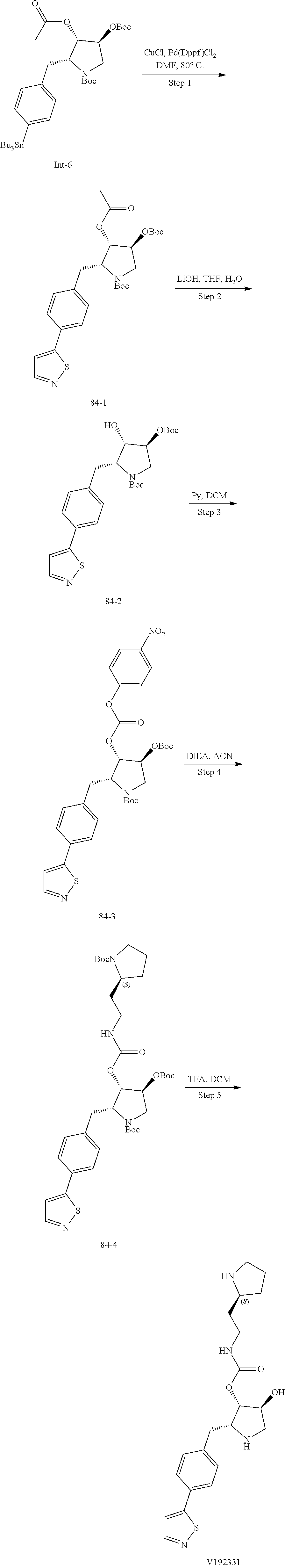

### Step 1 and 2

The compound was prepared in 55.9% overall yield as a light yellow oil according to Stille Coupling with Pd(PPh₃)₄, CuI and CsF; General Procedure IV using 5-bromo-1,2-thiazole in STEP 1; Hydrolysis reaction with LiOH; General Procedure V using tert-butyl (2R,3S,4S)-3-(acetyloxy)-4-[(tert-butoxycarbonyl)oxy]-2-{[4-(1,2-thiazol-5-yl)phenyl]methyl}pyrrolidine-1-carboxylate (84-1) instead of tert-butyl (2R,3S,4S)-3-(acetyloxy)-4-[(tert-butoxycarbonyl)oxy]-2-[(4-fluorophenyl)methyl]pyrrolidine-1-carboxylate (3-1) in STEP 2. MS: m/z Calc'd for C₂₄H₃₂N₂O₆S [M+H]⁺477. found 477.

Step 3: To the solution of tert-butyl (2R,3S,4S)-4-[(tert-butoxycarbonyl)oxy]-3-hydroxy-2-{[4-(1,2-thiazol-5-yl)phenyl]methyl}pyrrolidine-1-carboxylate (300 mg, 0.63 mmol, 1 equiv) and 4-nitrophenyl carbonochloridate (254 mg, 1.26 mmol, 2 equiv) in DCM (5 mL) was added pyridine (100 mg, 1.26 mmol, 2 equiv). The mixture was stirred at rt for 2 hours. The mixture was concentrated to give the crude product which was purified by reversed-phase flash chromatography to afford tert-butyl (2R,3S,4S)-4-[(tert-butoxycarbonyl)oxy]-3-[(4-nitrophenoxycarbonyl)oxy]-2-{[4-(1,2-thiazol-5-yl)phenyl]methyl}pyrrolidine-1-carboxylate (210 mg, 52.0% yield) as a light yellow solid. MS: m/z: C₃₁H₃₅N₃O₁₀S [M+H]⁺642. found 642.

### Step 4 and 5

The title compound was prepared in 48.9% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,2-thiazol-5-yl)phenyl]methyl}pyrrolidine-1-carboxylate (84-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate in STEP 4; Boc Deprotection; General Procedure II in STEP 5. MS: m/z Calc'd for C₂₁H₂₈N₄O₃S [M+H]⁺417. found 417. ¹H NMR (400 MHz, Methanol-d₄) δ 8.51 (d, J=1.8 Hz, 1H), 7.77-7.71 (m, 2H), 7.64 (d, J=1.8 Hz, 1H), 7.47 (d, J=8.1 Hz, 2H), 5.04-4.98 (m, 1H), 4.41 (d, J=4.1 Hz, 1H), 4.33-4.24 (m, 1H), 3.65-3.49 (m, 2H), 3.32-3.23 (m, 4H), 3.23 (d, J=7.1 Hz, 2H), 3.13 (dd, J=14.1, 8.6 Hz, 1H), 2.36-2.23 (m, 1H), 2.18-2.07 (m, 1H), 2.06-1.87 (m, 3H), 1.78-1.64 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 20 min; Wave Length: 254 nm/220 nm; RT1(min): 17.5.

### Synthetic Procedure of (2R,3S,4S)-4-hydroxy-2-{[4-(1,2-thiazol-5-yl)phenyl]methyl}pyrrolidin-3-yl N-[(3-fluorophenyl)methyl]carbamate (V192324)

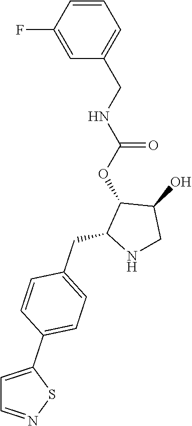 [SMILES: O=C(NCc1cccc(F)c1)O[C@@H]1[C@@H](O)CN[C@@H]1Cc1ccc(-c2ccns2)cc1]

The title compound was prepared in 36.7% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,2-thiazol-5-yl)phenyl]methyl}pyrrolidine-1-carboxylate (84-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z Calc'd for C₂₂H₂₂FN₃O₃S[M+H]⁺428. found 428. ¹H NMR (400 MHz, Methanol-d₄) δ 8.51 (d, J=1.9 Hz, 1H), 7.71-7.64 (m, 2H), 7.61 (d, J=1.9 Hz, 1H), 7.45-7.33 (m, 3H), 7.17 (d, J=7.6 Hz, 1H), 7.14-7.08 (m, 1H), 7.08-6.99 (m, 1H), 4.99-4.93 (m, 1H), 4.48-4.36 (m, 2H), 4.35-4.23 (m, 2H), 3.62 (dd, J=12.7, 4.3 Hz, 1H), 3.30-3.18 (m, 2H), 3.18-3.08 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 15% B to 45% B in 10 min; Wave Length: 254 nm/220 nm; RT1(min): 8.5.

### Synthetic Procedure of (2R,3S,4S)-2-{[4-(difluoromethyl)phenyl]methyl}-4-hydroxypyrrolidin-3-yl N-[(3-fluorophenyl)methyl]carbamate (V192274)

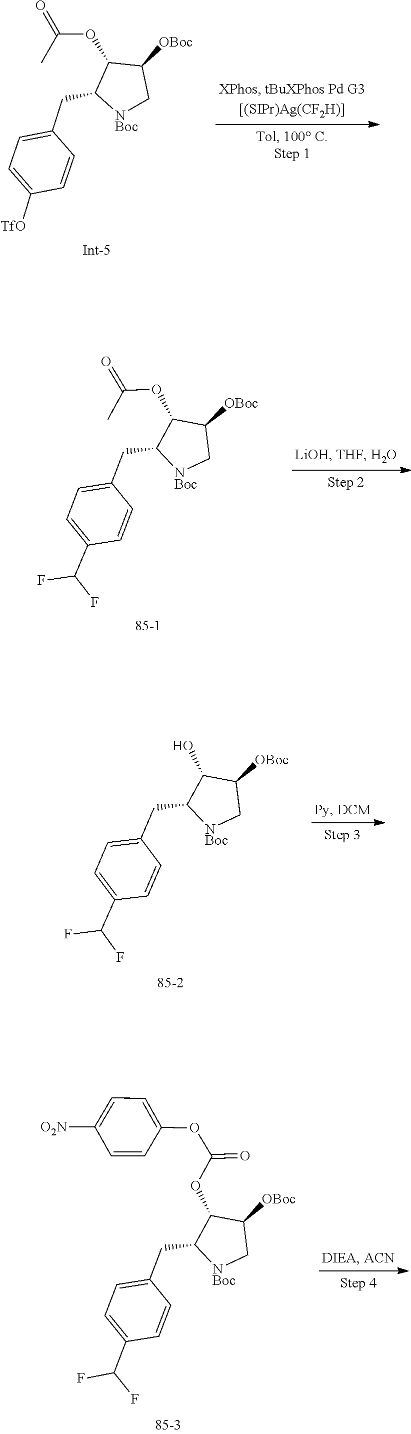

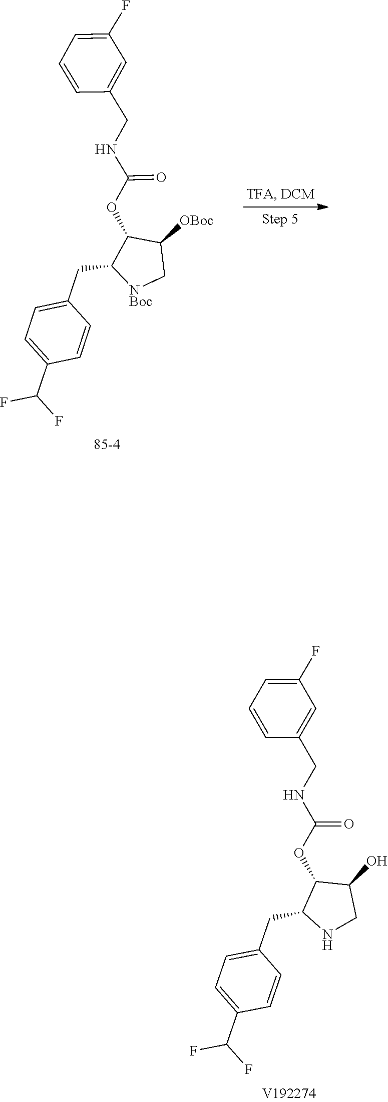

Step 1: To the solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (105 mg, 0.18 mmol, 1 equiv) and [1,3-bis(2,6-diisopropylphenyl)imidazolidin-2-ylidene](difluoromethyl)silver (129 mg, 0.23 mmol, 1.3 equiv) in toluene (5 mL) was added XPhos (9 mg, 0.018 mmol, 0.1 equiv) and t-BuXPhos Pd G3 (14 mg, 0.018 mmol, 0.1 equiv). The mixture was stirred at 100° C. for 7 hours under N₂. The mixture was diluted with EA, washed with water, dried and concentrated to give the crude product used directly in the next step. The tert-butyl (2R,3S,4S)-3-(acetyloxy)-4-[(tert-butoxycarbonyl)oxy]-2-{[4-(difluoromethyl)phenyl]methyl}pyrrolidine-1-carboxylate (80 mg, 91.6% yield) was obtained as a yellow oil. MS: m/z: Calc'd for C₂₄H₃₃F₂NO₇ [M+H−56−56]⁺373. found 373.

Step 2: The compound was prepared in 87.6% yield as a light yellow oil according to Hydrolysis reaction with LiOH; General Procedure V. MS: m/z Calc'd for C₂₂H₃₁F₂NO₆ [M+H]⁺444. found 444.

Step 3: To the solution of tert-butyl (2R,3S,4S)-4-[(tert-butoxycarbonyl)oxy]-2-{[4-(difluoromethyl)phenyl]methyl}-3-hydroxypyrrolidine-1-carboxylate (60 mg, 0.14 mmol, 1 equiv) and 4-nitrophenyl carbonochloridate (55 mg, 0.27 mmol, 2 equiv) in DCM (3 mL) was added pyridine (21 mg, 0.27 mmol, 2 equiv). The mixture was stirred at rt for 2 hours. The mixture was concentrated to give the crude product which was purified by reversed-phase flash chromatography to afford tert-butyl (2R,3S,4S)-4-[(tert-butoxycarbonyl)oxy]-2-{[4-(difluoromethyl)phenyl]methyl}-3-[(4-nitrophenoxycarbonyl)oxy]pyrrolidine-1-carboxylate (40 mg, 48.6% yield) as a white solid. MS: m/z: C₂₉H₃₄F₂N₂O₁₀ [M+H−56−56]⁺497. found 497.

### Step 4 and 5

The title compound was prepared in 48.9% overall yield as a white solid according to Substitution reaction for synthesis of Carbamate; General Procedure I using tert-butyl (2R,3S,4S)-4-[(tert-butoxycarbonyl)oxy]-2-{[4-(difluoromethyl)phenyl]methyl}-3-[(4-nitrophenoxycarbonyl)oxy]pyrrolidine-1-carboxylate (85-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 4; Boc Deprotection; General Procedure II in STEP 5. MS: m/z Calc'd for C₂₀H₂₁F₃N₂O₃[M+H]⁺395. found 395. ¹H NMR (400 MHz, Methanol-d₄) δ 7.44 (d, J=7.9 Hz, 3H), 7.40-7.30 (m, 3H), 7.15 (d, J=7.7 Hz, 1H), 7.08 (d, J=10.0 Hz, 1H), 7.06-6.97 (m, 1H), 6.72 (t, J=56.4 Hz, 1H), 4.64 (d, J=3.7 Hz, 1H), 4.42-4.24 (m, 2H), 4.17 (d, J=5.2 Hz, 1H), 3.66-3.57 (m, 1H), 3.41-3.35 (m, 1H), 3.00-2.85 (m, 2H), 2.80-2.71 (m, 1H).

Prep-HPLC purification conditions: Column: XBridge Prep OBD C18 Column, 30*150 mm, 5 μm; Mobile Phase A: Water (10 nmol/LNH₄HCO₃), Mobile Phase B: ACN; Flow rate: 60 mL/min; Gradient: 22% B to 52% B in 10 min; Wave Length: 254 nm/220 nm; RT1(min): 8.5.

### Synthetic Procedure of (2R,3S,4S)-2-{[4-(difluoromethyl)phenyl]methyl}-4-hydroxypyrrolidin-3-yl N-{2-[(2S)-azetidin-2-yl]ethyl}carbamate (V193008)

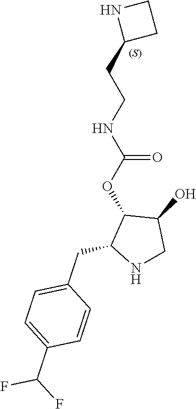

The title compound was prepared in 40.0% overall yield as a light yellow solid according to Substitution reaction for synthesis of Carbamate; General Procedure I using tert-butyl (2R,3S,4S)-4-[(tert-butoxycarbonyl)oxy]-2-{[4-(difluoromethyl)phenyl]methyl}-3-[(4-nitrophenoxycarbonyl)oxy]pyrrolidine-1-carboxylate (85-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2R)-2-(2-aminoethyl)azetidine-1-carboxylate in STEP 1; Boc Deprotection; General Procedure II in STEP 2. MS: m/z Calc'd for C₁₈H₂₅F₂N₃O₃[M+H]⁺370, found 370. ¹H NMR (400 MHz, Methanol-d₄) δ 7.57 (d, J=7.9 Hz, 2H), 7.48 (d, J=8.0 Hz, 2H), 6.79 (t, J=56.2 Hz, 1H), 4.98 (d, J=3.5 Hz, 1H), 4.58-4.45 (m, 1H), 4.40 (d, J=4.2 Hz, 1H), 4.31-4.22 (m, 1H), 4.07 (q, J=9.4 Hz, 1H), 3.97-3.86 (m, 1H), 3.60 (dd, J=12.7, 4.3 Hz, 1H), 3.32-3.18 (m, 4H), 3.14 (dd, J=14.2, 8.6 Hz, 1H), 2.69-2.56 (m, 1H), 2.46-2.32 (m, 1H), 2.24-2.02 (m, 2H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 24% B in 10 min; Wave Length: 254 nm/220 nm; RT1(min): 8.5.

### Synthetic Procedure of: (2R,3S,4S)-2-{[4-(difluoromethyl)phenyl]methyl}-4-hydroxypyrrolidin-3-yl N-[(3-fluorophenyl)methyl]carbamate (V192508)

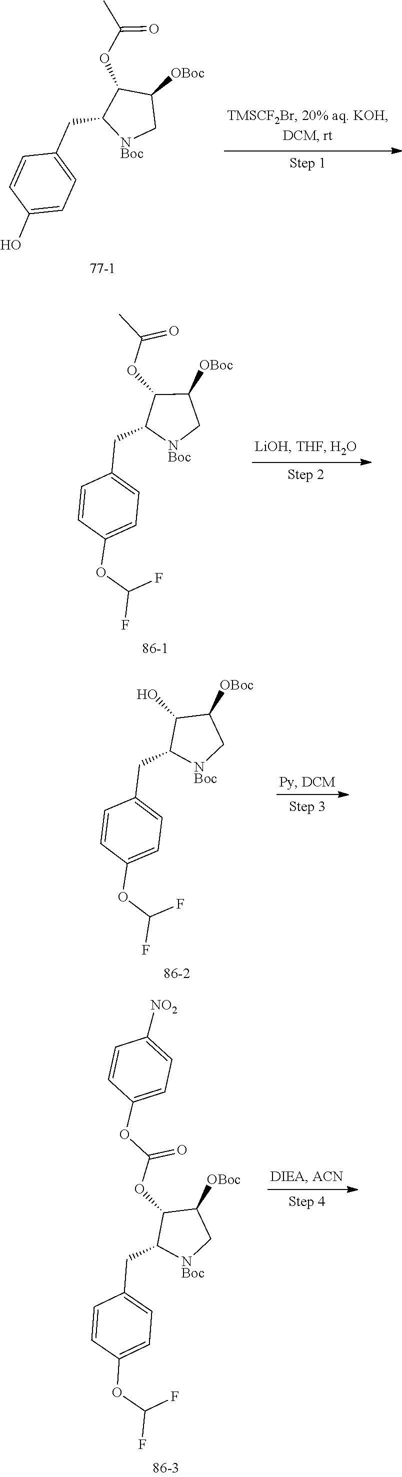

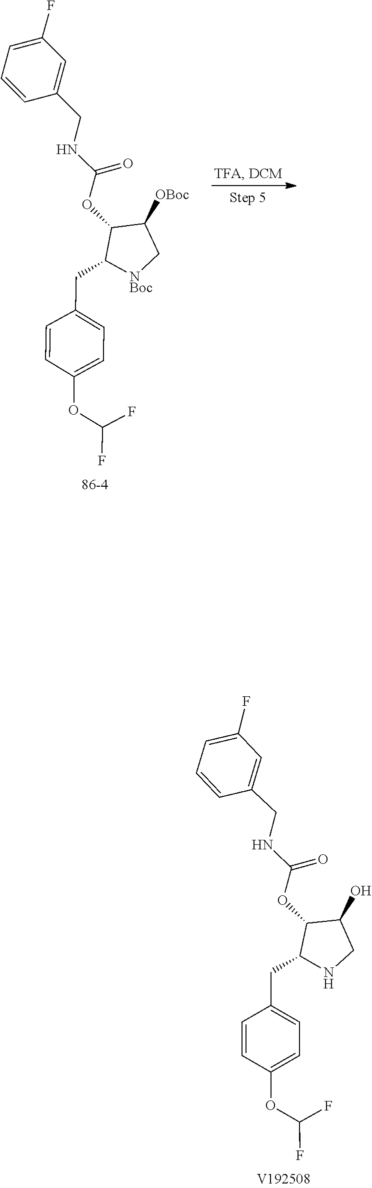

Step 1: To the solution of tert-butyl (2R,3S,4S)-3-(acetyloxy)-4-[(tert-butoxycarbonyl)oxy]-2-[(4-hydroxyphenyl)methyl]pyrrolidine-1-carboxylate (200 mg, 0.44 mmol, 1 equiv) in DCM (10 mL) was added KOH (149 mg, 2.66 mmol, 6 equiv) (20% aq.). Then (bromodifluoromethyl)trimethylsilane (180 mg, 0.89 mmol, 2 equiv) was added. The mixture was stirred at rt for 1 hour. The mixture was diluted with DCM, washed with water, dried and concentrated. The crude product was used directly in the next step without further purification. The tert-butyl (2R,3S,4S)-3-(acetyloxy)-4-[(tert-butoxycarbonyl)oxy]-2-{[4-(difluoromethoxy)phenyl]methyl}pyrrolidine-1-carboxylate (160 mg, 72.0% yield) was obtained as a light yellow oil. MS: m/z: Calc'd for C₂₄H₃₃F₂NO₈ [M+H]⁺502. found 502.

Step 2: The compound was prepared in 90.9% yield as a brown oil according to Hydrolysis reaction with LiOH; General Procedure V. MS: m/z Calc'd for C₂₂H₃₁F₂NO₇ [M+Na]⁺460. found 460.

Step 3: To the solution of tert-butyl (2R,3S,4S)-4-[(tert-butoxycarbonyl)oxy]-2-{[4-(difluoromethoxy)phenyl]methyl}-3-hydroxypyrrolidine-1-carboxylate (200 mg, 0.44 mmol, 1 equiv) and 4-nitrophenyl carbonochloridate (175 mg, 0.87 mmol, 2 equiv) in DCM (5 mL) was added pyridine (69 mg, 0.87 mmol, 2 equiv). The mixture was stirred at rt for 2 hours. The mixture was concentrated to give the crude product which was purified by reversed-phase flash chromatography to afford tert-butyl (2R,3S,4S)-4-[(tert-butoxycarbonyl)oxy]-2-{[4-(difluoromethoxy)phenyl]methyl}-3-[(4-nitrophenoxycarbonyl)oxy]pyrrolidine-1-carboxylate (120 mg, 44.1% yield) as a colorless oil. MS: m/z: Calc'd for C₂₉H₃₄F₂N₂O [M+Na]⁺625. found 625.

Step 4 and 5: The title compound was prepared in 37.7% overall yield as a grey solid according to Substitution reaction for synthesis of Carbamate; General Procedure I using tert-butyl (2R,3S,4S)-4-[(tert-butoxycarbonyl)oxy]-2-{[4-(difluoromethoxy)phenyl]methyl}-3-[(4-nitrophenoxycarbonyl)oxy]pyrrolidine-1-carboxylate (86-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 4; Boc Deprotection; General Procedure II in STEP 5. MS: m/z Calc'd for C₂₀H₂₁F₃N₂O₄[M+H]⁺411. found 411. ¹H NMR (400 MHz, Methanol-d₄) δ 7.43-7.33 (m, 1H), 7.34 (d, J=8.5 Hz, 2H), 7.19-7.06 (m, 4H), 7.08-6.98 (m, 1H), 6.72 (d, J=74.0 Hz, 1H), 4.96 (d, J=3.6 Hz, 1H), 4.44-4.35 (m, 2H), 4.31 (d, J=15.5 Hz, 1H), 4.26-4.17 (m, 1H), 3.59 (dd, J=12.6, 4.3 Hz, 1H), 3.28-3.13 (m, 2H), 3.10-3.00 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 18% B to 48% B in 10 min; Wave Length: 254 nm/220 nm; RT1(min): 8.5.

### Synthetic Procedure of (2R,3S,4S)-2-{[4-(difluoromethoxy)phenyl]methyl}-4-hydroxypyrrolidin-3-yl N-{2-[(2S)-azetidin-2-yl]ethyl}carbamate (V192938)

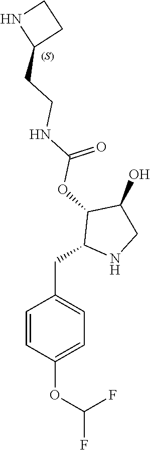

The title compound was prepared in 31.2% overall yield as a grey solid according to Substitution reaction for synthesis of Carbamate; General Procedure I using tert-butyl (2R,3S,4S)-4-[(tert-butoxycarbonyl)oxy]-2-{[4-(difluoromethoxy)phenyl]methyl}-3-[(4-nitrophenoxycarbonyl)oxy]pyrrolidine-1-carboxylate (86-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)azetidine-1-carboxylate in STEP 1; Boc Deprotection; General Procedure II in STEP 2. MS: m/z Calc'd for C₁₈H₂₅F₂N₃O₄[M+H]⁺386, found 386. ¹H NMR (400 MHz, Methanol-d₄) δ 7.38 (d, J=8.4 Hz, 2H), 7.17 (d, J=8.4 Hz, 2H), 6.83 (t, J=74.0 Hz, 1H), 4.98 (d, J=3.6 Hz, 1H), 4.57-4.45 (m, 1H), 4.40 (d, J=4.2 Hz, 1H), 4.27-4.18 (m, 1H), 4.13-4.02 (m, 1H), 3.97-3.87 (m, 1H), 3.58 (dd, J=12.6, 4.3 Hz, 1H), 3.29-3.13 (m, 3H), 3.06 (dd, J=14.2, 8.7 Hz, 1H), 2.69-2.56 (m, 1H), 2.46-2.32 (m, 1H), 2.24-2.02 (m, 2H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 27% B in 10 min; Wave Length: 254 nm/220 nm; RT1(min): 8.5.

### Synthetic Procedure of (2R,3S,4S)-2-{[1,1′-biphenyl]-4-ylmethyl}-4-hydroxypyrrolidin-3-yl N-{2-[(2S)-pyrrolidin-2-yl]ethyl}carbamate (V190505)

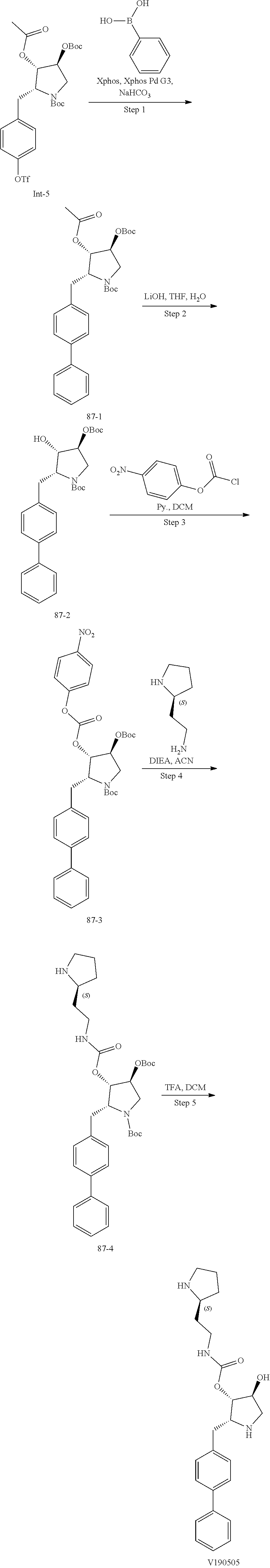

Step 1: To a stirred solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (Int-5, 300 mg, 0.51 mmol, 1.0 eq.) and phenyl boronic acid (75.2 mg, 0.62 mmol, 1.2 eq.) in DMF (10 mL) were added XPhos Pd G₃ (43.5 mg, 0.05 mmol, 0.1 eq.), XPhos (49.0 mg, 0.10 mmol, 0.2 eq.) and NaHCO₃ (86.4 mg, 1.03 mmol, 2.0 eq.) at room temperature. The resulting mixture was stirred at 80° C. for 12 h under nitrogen atmosphere. Desired product could be detected by LCMS. The residue was purified by reversed-phase flash chromatography to afford tert-butyl (2R,3S,4S)-3-(acetyloxy)-2-{[1,1′-biphenyl]-4-ylmethyl}-4-[(tert-butoxycarbonyl)oxy]pyrrolidine-1-carboxylate (230 mg, 87.5%) as a light yellow oil. MS: m/z: Calc'd for C₂₉H₃₇NO₇ [M+H−56−56]⁺400. Found, 400.

Step 2: The compound was prepared in 94.7% yield as a light yellow oil according to Hydrolysis reaction with LiOH; General Procedure V. MS: m/z Calc'd for C₂₇H₃₅NO₆ [M+H−56−56]⁺358. Found, 358.

Step 3: To a stirred solution of tert-butyl (2R,3S,4S)-2-{[1,1′-biphenyl]-4-ylmethyl}-4-[(tert-butoxycarbonyl)oxy]-3-hydroxypyrrolidine-1-carboxylate (190 mg, 0.41 mmol, 1.0 eq.) and 4-nitrophenyl carbonochloridate (122.3 mg, 0.61 mmol, 1.5 eq.) in DCM (5 mL) was added Pyridine (64.0 mg, 0.81 mmol, 2.0 eq.) in portions at room temperature. The resulting mixture was stirred over night at 25° C. Desired product could be detected by LCMS. The residue was purified by Prep-TLC (PE/EA 2:1) to afford tert-butyl (2R,3S,4S)-2-{[1,1′-biphenyl]-4-ylmethyl}-4-[(tert-butoxycarbonyl)oxy]-3-[(4-nitrophenoxycarbonyl)oxy]pyrrolidine-1-carboxylate (200 mg, 77.9%) as a light yellow oil. MS: m/z: Calc'd for C₃₄H₃₈N₂O₁₀ [M+H−100]⁺535. Found, 535.

### Step 4 and 5

The title compound was prepared in 41.2% overall yield as a white solid according to Substitution reaction for synthesis of Carbamate; General Procedure I using tert-butyl (2R,3S,4S)-2-{[1,1′-biphenyl]-4-ylmethyl}-4-[(tert-butoxycarbonyl)oxy]-3-[(4-nitrophenoxycarbonyl)oxy]pyrrolidine-1-carboxylate (87-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate in STEP 4; Boc Deprotection; General Procedure II in STEP 5. MS: m/z Calc'd for C₂₄H₃₁N₃O₃ [M+H]⁺410. Found, 410. ¹H NMR (400 MHz, Methanol-d₄) δ 7.63 (dd, J=9.8, 7.5 Hz, 4H), 7.47-7.42 (m, 4H), 7.38-7.34 (m, 1H), 5.01 (d, J=3.5 Hz, 1H), 4.41 (d, J=4.1 Hz, 1H), 4.30-4.25 (m, 1H), 3.64-3.52 (m, 2H), 3.36 (s, 2H), 3.31-3.20 (m, 4H), 3.12 (dd, J=14.4, 8.6 Hz, 1H), 2.32-2.25 (m, 1H), 2.15-1.90 (m, 4H), 1.76-1.66 (m, 1H).

Prep-HPLC purification conditions: Column: Welch Ultimate PFP-C18 30*250, 10 μm; Mobile Phase A: Water (0.05% TFA), Mobile Phase B: ACN; Flow rate: 60 mL/min; Gradient: 2% B to 30% B in 10 min; Wave Length: 254 nm/220 nm; RT1(min): 10.48.

### Synthetic Procedure of (2R,3S,4S)-2-{[1,1′-biphenyl]-4-ylmethyl}-4-hydroxypyrrolidin-3-yl N-{2-[(2R)-pyrrolidin-2-yl]ethyl}carbamate (V192036)

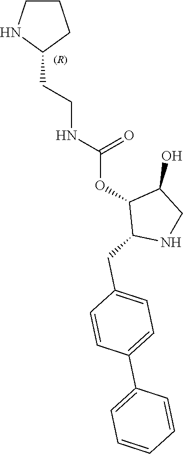

The title compound was prepared in 38.8% overall yield as a white solid according to Substitution reaction for synthesis of Carbamate; General Procedure I using tert-butyl (2R,3S,4S)-2-{[1,1′-biphenyl]-4-ylmethyl}-4-[(tert-butoxycarbonyl)oxy]-3-[(4-nitrophenoxycarbonyl)oxy]pyrrolidine-1-carboxylate (87-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (2R)-2-(2-aminoethyl)pyrrolidine-1-carboxylate in STEP 1; Boc Deprotection; General Procedure II in STEP 2. MS: m/z Calc'd for C₂₄H₃₁N₃O₃ [M+H]⁺410. found 410. ¹H NMR (400 MHz, Methanol-d₄) δ 7.68-7.58 (m, 4H), 7.55-7.31 (m, 5H), 5.06-5.01 (m, 1H), 4.42 (d, J=4.1 Hz, 1H), 4.27-4.25 (m, 1H), 3.64-3.52 (m, 2H), 3.33-3.27 (m, 3H), 3.27-3.18 (m, 2H), 3.16-3.12 (m, 1H), 3.09 (dd, J=14.1, 8.6 Hz, 1H), 2.30 (dd, J=14.3, 7.3, Hz, 1H), 2.10-1.91 (m, 4H), 1.71 (dd, J=13.0, 8.9 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 6% B to 36% B in 10 min; Wave Length: 254 nm/220 nm; RT1(min): 9.3.

### Synthetic Procedure of (2R,3S,4S)-2-{[1,1′-biphenyl]-4-ylmethyl}-4-hydroxypyrrolidin-3-yl N-[2-(piperidin-4-yl)ethyl]carbamate (V192050)

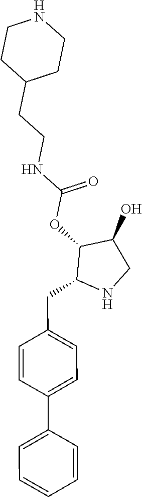 [SMILES: O=C(NCCC1CCNCC1)O[C@@H]1[C@@H](O)CN[C@@H]1Cc1ccc(-c2ccccc2)cc1]

The title compound was prepared in 46.6% overall yield as a white solid according to Substitution reaction for synthesis of Carbamate; General Procedure I using tert-butyl (2R,3S,4S)-2-{[1,1′-biphenyl]-4-ylmethyl}-4-[(tert-butoxycarbonyl)oxy]-3-[(4-nitrophenoxycarbonyl)oxy]pyrrolidine-1-carboxylate (87-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 4-(2-aminoethyl)piperidine-1-carboxylate in STEP 1; Boc Deprotection; General Procedure II in STEP 2. MS: m/z Calc'd for C₂₅H₃₃N₃O₃ [M+H]⁺424. Found, 424. ¹H NMR (400 MHz, Methanol-d₄) δ 7.68-7.59 (m, 4H), 7.50-7.34 (m, 5H), 5.00 (d, J=3.5 Hz, 1H), 4.40 (d, J=4.1 Hz, 1H), 4.28-4.24 (m, 1H), 3.63-3.58 (m, 1H), 3.44-3.35 (m, 2H), 3.23 (dd, J=12.9, 6.9 Hz, 4H), 3.13-3.06 (m, 1H), 3.01-2.93 (m, 2H), 2.01 (d, J=14.2 Hz, 2H), 1.74-1.65 (m, 1H), 1.60-1.54 (m, 2H), 1.50-1.35 (m, 2H).

Prep-HPLC purification conditions: Column: Welch Ultimate PFP-C18 30*250, 10 μm; Mobile Phase A: Water (0.05% TFA), Mobile Phase B: ACN; Flow rate: 60 mL/min; Gradient: 2% B to 31% B in 10 min; Wave Length: 254 nm/220 nm; RT1(min): 10.48.

### Synthetic Procedure of 2R,3S,4S)-4-hydroxy-2-[(4-{imidazo[1,2-a]pyridin-2-yl}phenyl)methyl]pyrrolidin-3-yl N-[(3-fluorophenyl)methyl]carbamate (V192139)

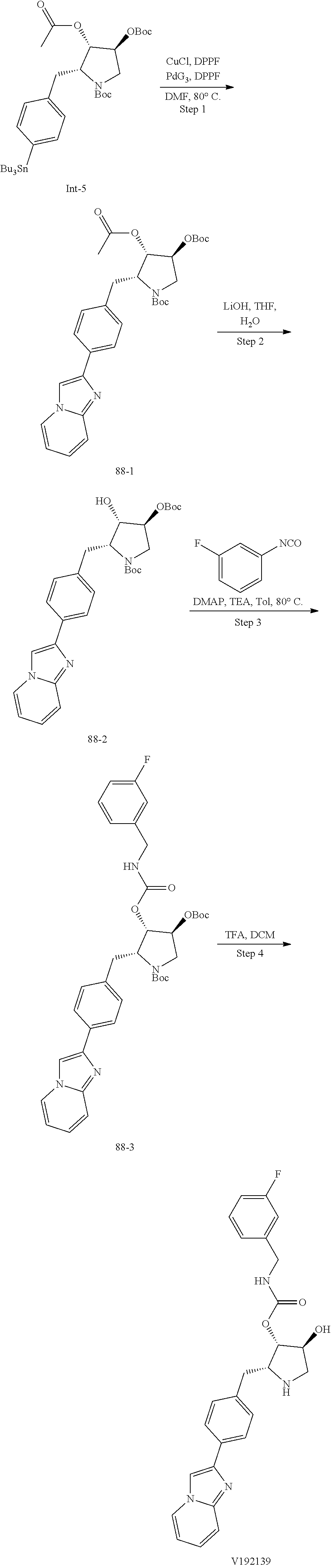

Step 1: To the solution of tert-butyl (2R,3S,4S)-3-(acetyloxy)-4-[(tert-butoxycarbonyl)oxy]-2-{[4-(tributylstannyl)phenyl]methyl}pyrrolidine-1-carboxylate (Int-5, 400 mg, 0.55 mmol, 1 equiv), 2-bromoimidazo[1,2-a]pyridine (163 mg, 0.83 mmol, 1.5 equiv) and Dppf (61 mg, 0.11 mmol, 0.2 equiv) in DMF (10 mL) was added CuCl (109 mg, 1.104 mmol, 2 equiv) and Dppf palladium(II) biphenyl-2-amine (51 mg, 0.055 mmol, 0.1 equiv). The mixture was stirred at 80° C. for overnight under N₂. The mixture was diluted with EA, washed with water, dried and concentrated. The crude product was purified by Prep TLC (PE:EA=1:1) to afford tert-butyl (2R,3S,4S)-3-(acetyloxy)-4-[(tert-butoxycarbonyl)oxy]-2-[(4-{imidazo[1,2-a]pyridin-2-yl}phenyl)methyl]pyrrolidine-1-carboxylate (140 mg, 46.0% yield) as a light yellow solid. MS: m/z: Calc'd for C₃₀H₃₇N₃O₇ [M+H]⁺552. found 552.

### Step 2, 3, 4 and 5

The title compound was prepared in 23.8% overall yield as a white solid according to Hydrolysis reaction with LiOH; General Procedure VII using tert-butyl (2R,3S,4S)-3-(acetyloxy)-4-[(tert-butoxycarbonyl)oxy]-2-[(4-{imidazo[1,2-a]pyridin-2-yl}phenyl)methyl]pyrrolidine-1-carboxylate (88-1) instead of tert-butyl (2R,3S,4S)-3-(acetyloxy)-4-[(tert-butoxycarbonyl)oxy]-2-[(4-fluorophenyl)methyl]pyrrolidine-1-carboxylate (3-1) in STEP 2; Addition reaction: Isocyanates and hydroxyl groups; General Procedure VI using tert-butyl (2R,3S,4S)-4-[(tert-butoxycarbonyl)oxy]-3-hydroxy-2-[(4-{imidazo[1,2-a]pyridin-2-yl}phenyl)methyl]pyrrolidine-1-carboxylate (88-2) instead of tert-butyl (2R,3S,4S)-4-[(tert-butoxycarbonyl)oxy]-2-[(4-fluorophenyl)methyl]-3-hydroxypyrrolidine-1-carboxylate (Int-7), 1-fluoro-3-isocyanatobenzene in STEP 3; Boc Deprotection; General Procedure II in STEP 4. MS: m/z Calc'd for C₂₆H₂₅FN₄O₃[M+H]⁺461. found 461. ¹H NMR (400 MHz, Methanol-d₄) δ 8.73 (d, J=6.8 Hz, 1H), 8.49 (s, 1H), 7.91 (d, J=7.9 Hz, 2H), 7.85 (d, J=4.0 Hz, 2H), 7.53 (d, J=7.9 Hz, 2H), 7.39-7.37 (m, 2H), 7.17 (d, J=7.9 Hz, 1H), 7.10 (d, J=10.0 Hz, 1H), 7.07-7.00 (m, 1H), 5.03-4.97 (m, 1H), 4.50-4.37 (m, 2H), 4.37-4.26 (m, 2H), 3.63 (dd, J=12.7, 4.3 Hz, 1H), 3.32-3.15 (m, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 4% B to 34% B in 10 min; Wave Length: 254 nm/220 nm; RT1(min): 8.5.

### Synthetic Procedure of (2R,3S,4S)-4-hydroxy-2-({4-[5-(trifluoromethyl)-1,3,4-thiadiazol-2-yl]phenyl}methyl)pyrrolidin-3-yl N-[(3-fluorophenyl)methyl]carbamate (V192102)

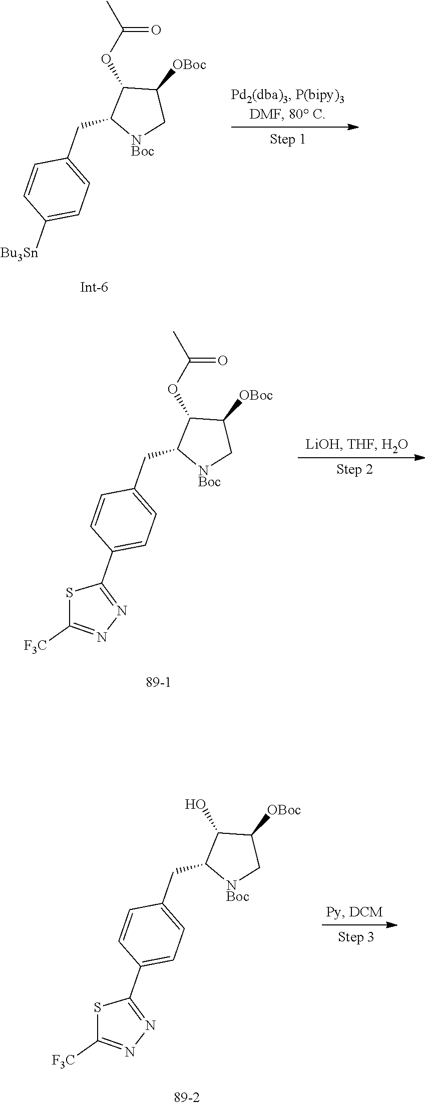

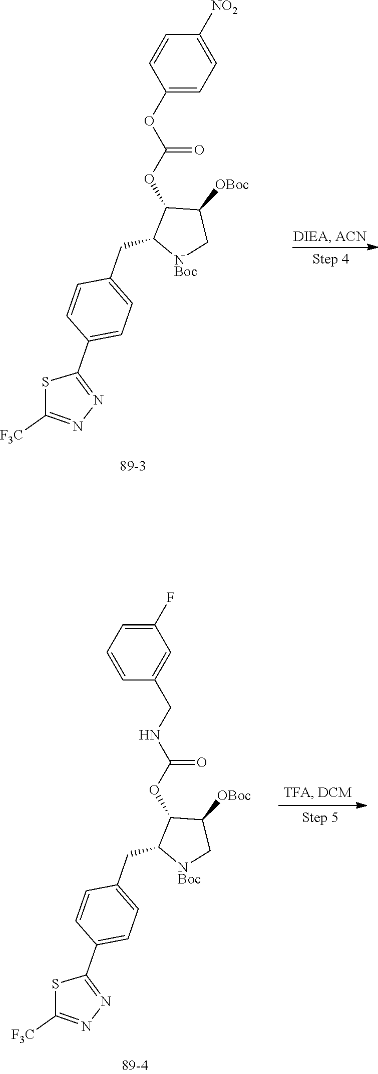

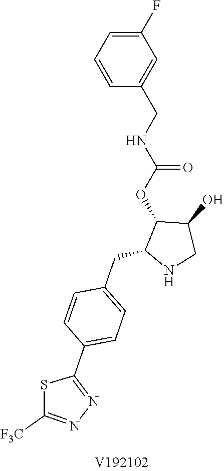 [SMILES: O=C(NCc1cccc(F)c1)O[C@@H]1[C@@H](O)CN[C@@H]1Cc1ccc(-c2nnc(C(F)(F)F)s2)cc1]

Step 1: To the solution of tert-butyl (2R,3S,4S)-3-(acetyloxy)-4-[(tert-butoxycarbonyl)oxy]-2-{[4-(tributylstannyl)phenyl]methyl}pyrrolidine-1-carboxylate (Int-6, 110 mg, 0.15 mmol, 1 equiv) and 2-bromo-5-(trifluoromethyl)-1,3,4-thiadiazole (53 mg, 0.23 mmol, 1.5 equiv) in DMF (3 mL) was added Pd₂(dba)₃ (14 mg, 0.015 mmol, 0.1 equiv) and tris(furan-2-yl)phosphane (7 mg, 0.030 mmol, 0.2 equiv). The mixture was stirred at 80° C. for overnight under N₂. The mixture was filtered and the filtrate was purified by reversed-phase flash chromatography to afford tert-butyl (2R,3S,4S)-3-(acetyloxy)-4-[(tert-butoxycarbonyl)oxy]-2-({4-[5-(trifluoromethyl)-1,3,4-thiadiazol-2-yl]phenyl}methyl)pyrrolidine-1-carboxylate (40 mg, 44.8%) was obtained as a colorless oil. MS: m/z: Calc'd for C₂₆H₃₂F₃N₃O₇S [M+H−56−56]⁺476. found 476.

Step 2: The compound was prepared in 89.8% yield as a brown oil according to Hydrolysis reaction with LiOH; General Procedure V. MS: m/z Calc'd for C₂₄H₃₀F₃N₃O₆S [M+Na]⁺568. found 568.

Step 3: To the solution of tert-butyl (2R,3S,4S)-4-[(tert-butoxycarbonyl)oxy]-3-hydroxy-2-({4-[5-(trifluoromethyl)-1,3,4-thiadiazol-2-yl]phenyl}methyl)pyrrolidine-1-carboxylate (50 mg, 0.092 mmol, 1 equiv) and 4-nitrophenyl carbonochloridate (37 mg, 0.18 mmol, 2 equiv) in DCM (3 mL) was added Pyridine (15 mg, 0.18 mmol, 2 equiv). The mixture was stirred at rt for 2 hours. The mixture was concentrated to give the crude product which was purified by reversed-phase flash chromatography to afford tert-butyl (2R,3S,4S)-4-[(tert-butoxycarbonyl)oxy]-3-[(4-nitrophenoxycarbonyl)oxy]-2-({4-[5-(trifluoromethyl)-1,3,4-thiadiazol-2-yl]phenyl}methyl)pyrrolidine-1-carboxylate (48 mg, 73.7%) as a light yellow oil. MS: m/z: Calc'd for C₃₁H₃₃F₃N₄O₁₀s [M+Na]⁺733. found 733.

### Step 4 and 5

The title compound was prepared in 26.9% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-({4-[5-(trifluoromethyl)-1,3,4-thiadiazol-2-yl]phenyl}methyl)pyrrolidine-1-carboxylate (89-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 4; Boc Deprotection; General Procedure II in STEP 5. MS: m/z Calc'd for C₂₂H₂₀F₄N₄O₃S[M+H]⁺497. found 497. ¹H NMR (400 MHz, Methanol-d₄) δ 8.09-8.03 (m, 2H), 7.53 (d, J=8.1 Hz, 2H), 7.44-7.34 (m, 1H), 7.17 (d, J=7.6 Hz, 1H), 7.13-6.99 (m, 2H), 5.02-4.96 (m, 1H), 4.46-4.36 (m, 2H), 4.36-4.27 (m, 2H), 3.62 (dd, J=12.7, 4.2 Hz, 1H), 3.32-3.24 (m, 2H), 3.23-3.13 (m, 1H).

Prep-HPLC purification conditions: Column: Xselect CSH Prep Fluoro-Phenyl Column, 30*150 mm, 5 μm; Mobile Phase A: Water (0.05% TFA), Mobile Phase B: ACN; Flow rate: 60 mL/min; Gradient: 10% B to 40% B in 10 min; Wave Length: 254 nm/220 nm; RT1(min): 9.68.

### Synthetic Procedure of (2R,3S,4S)-4-hydroxy-2-{[4-(1,3-oxazol-5-yl)phenyl]methyl}pyrrolidin-3-yl N-{[3-(trifluoromethoxy)phenyl]methyl}carbamate (V192276)

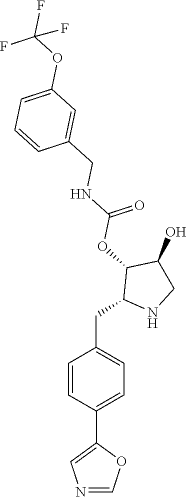 [SMILES: O=C(NCc1cccc(OC(F)(F)F)c1)O[C@@H]1[C@@H](O)CN[C@@H]1Cc1ccc(-c2cnco2)cc1]

The title compound was prepared in 46.9% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-[3-(trifluoromethoxy)phenyl]methanamine in STEP 1; Boc Deprotection; General Procedure II in STEP 2. MS: m/z Calc'd for C₂₃H₂₂F₃N₃O₅[M+H]⁺478. found 478. ¹H NMR (400 MHz, Methanol-d₄) δ 8.28 (s, 1H), 7.75-7.68 (m, 2H), 7.53 (s, 1H), 7.48 (d, J=7.9 Hz, 1H), 7.41-7.36 (d, J=8.1 Hz, 3H), 7.28 (s, 1H), 7.24 (d, J=8.1 Hz, 1H), 5.00 (d, J=3.5 Hz, 1H), 4.48-4.35 (m, 3H), 4.27 (dd, J=7.8, 3.5 Hz, 1H), 3.60 (dd, J=12.6, 4.3 Hz, 1H), 3.24 (dd, J=21.5, 10.0 Hz, 2H), 3.10 (dd, J=14.0, 8.3 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 17% B to 47% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-{2-[(2S,5S)-5-methylpyrrolidin-2-yl]ethyl}carbamate (V192850)

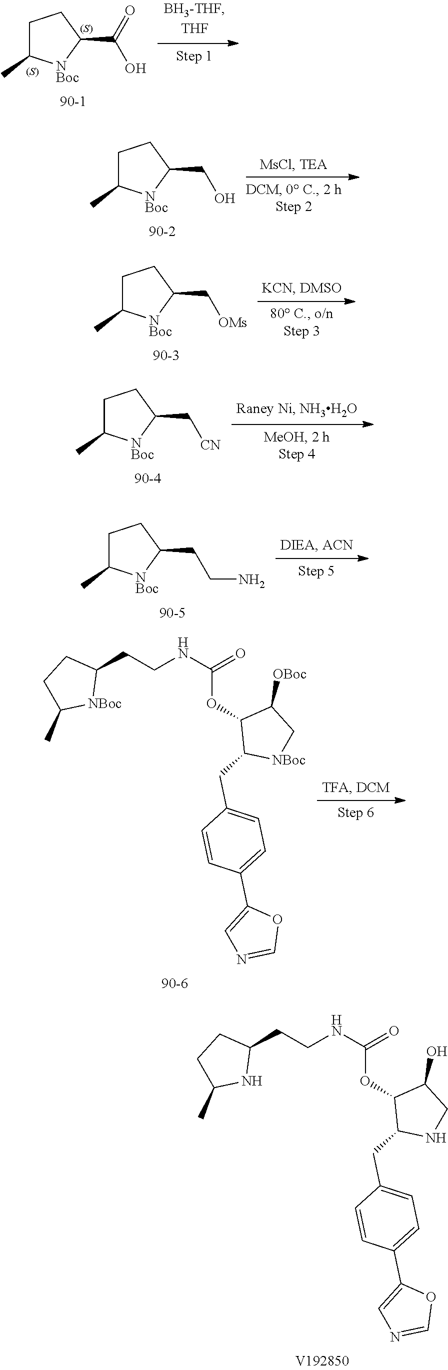

Step 1: To a stirred solution of (2S,5S)-1-(tert-butoxycarbonyl)-5-methylpyrrolidine-2-carboxylic acid (1 g, 4.36 mmol, 1 eq.) in THF (8 mL) were added Borane-tetrahydrofuran complex (1.0 M in THF) (8 mL) at 0° C. The resulting mixture was stirred at room temperature for overnight. The resulting mixture was concentrated under reduced pressure. The residue was purified by silica gel column chromatography, eluted with PE/EA (3:1) to afford tert-butyl (2S,5S)-2-(hydroxymethyl)-5-methylpyrrolidine-1-carboxylate (800 mg, 85.2% yield) as a light yellow oil. MS: m/z: Calc'd for C₁₁H₂₁NO₃ [M+H−56]⁺160. Found, 160.

Step 2: To a stirred solution of tert-butyl (2S,5S)-2-(hydroxymethyl)-5-methylpyrrolidine-1-carboxylate (600 mg, 2.78 mmol, 1 eq.) and methanesulfonyl chloride (638.4 mg, 5.57 mmol, 2 eq.) in DCM (8 mL) were added Et₃N (846.0 mg, 8.36 mmol, 3 eq.) at 0° C. The resulting mixture was stirred at room temperature for 2 h. The resulting mixture was concentrated under reduced pressure. The residue was purified by silica gel column chromatography, eluted with PE/EA (3:1) to afford tert-butyl (2S,5S)-2-[(methanesulfonyloxy)methyl]-5-methylpyrrolidine-1-carboxylate (800 mg, 97.8% yield, 95% purity) as a light yellow oil. MS: m/z: Calc'd for C₁₂H₂₃NO₅S [M+H−56]⁺238. Found, 238.

Step 3: To a stirred solution of tert-butyl (2S,5S)-2-[(methanesulfonyloxy)methyl]-5-methylpyrrolidine-1-carboxylate (790 mg, 2.69 mmol, 1 eq.) and KCN (350.6 mg, 5.38 mmol, 2 eq.) in DMSO (8 mL) at room temperature. The resulting mixture was stirred at 80° C. for overnight. The resulting mixture was extracted with EtOAc (2×100 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (6:1) to afford tert-butyl (2S,5S)-2-(cyanomethyl)-5-methylpyrrolidine-1-carboxylate (370 mg, 61.2% yield, 95% purity) as a light yellow oil. MS: m/z: Calc'd for C₁₂H₂₀N₂O₂ [M+H−56]⁺169. Found, 169.

Step 4: To a stirred solution of tert-butyl (2S,5S)-2-(cyanomethyl)-5-methylpyrrolidine-1-carboxylate (365 mg, 1.62 mmol, 1 eq.) and raney nickel (286.5 mg, 4.88 mmol, 3 eq.) in MEOH (6 mL) were added Ammonium hydroxide (9% in water) (85.5 mg, 2.44 mmol, 1.5 eq.) at room temperature. The resulting mixture was stirred at room temperature for 2 h. The precipitated solids were collected by filtration and washed with MEOH (6 mL) (2×30 mL). The resulting mixture was concentrated under reduced pressure to afford tert-butyl (2S,5S)-2-(2-aminoethyl)-5-methylpyrrolidine-1-carboxylate (250 mg) as a light yellow oil. MS: m/z: Calc'd for C₁₂H₂₄N₂O₂ [M+H]⁺229. Found, 229.

### Step 5 and 6

The title compound was prepared in 23.0% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), bis(tert-butyl (2S,5S)-2-(2-aminoethyl)-5-methylpyrrolidine-1-carboxylate) (90-5) in STEP 5; Boc Deprotection; General Procedure II in STEP 6. MS: m/z Calc'd for C₂₂H₃₀N₄O₄ [M+H]⁺415. found 415. ¹H NMR (400 MHz, Methanol-d₄) δ 8.28 (s, 1H), 7.80-7.73 (m, 2H), 7.54 (s, 1H), 7.45 (d, J=8.2 Hz, 2H), 5.05-4.99 (m, 1H), 4.41 (d, J=4.0 Hz, 1H), 4.33-4.24 (m, 1H), 3.71-3.64 (m, 1H), 3.61-3.51 (m, 2H), 3.32-3.18 (m, 4H), 3.10 (dd, J=14.2, 8.6 Hz, 1H), 2.29 (dd, J=16.1, 8.2 Hz, 2H), 2.09-1.83 (m, 2H), 1.77-1.75 (m, 2H), 1.43 (d, J=6.6 Hz, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 23% B in 10 min; Wave Length: 254 nm/220 nm; RT1(min): 9.5.

### Synthetic Procedure of (2R,3S,4S)-4-hydroxy-2-{[4-(1,3-oxazol-5-yl)phenyl]methyl}pyrrolidin-3-yl N-{2-[(2S,4S)-4-(trifluoromethyl)pyrrolidin-2-yl]ethyl}carbamate (V192912)

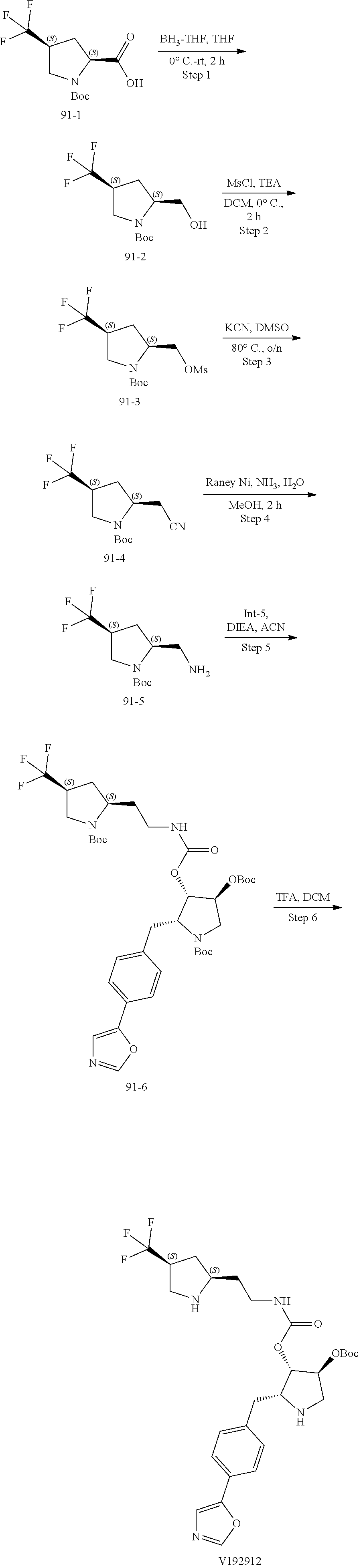

Step 1: To a stirred solution of (2S,4S)-1-(tert-butoxycarbonyl)-4-(trifluoromethyl)pyrrolidine-2-carboxylic acid (500 mg, 1.76 mmol, 1 eq.) in THF (4 mL) were added Borane-tetrahydrofuran complex (1.0 M in THF) (4 mL) at 0° C. The resulting mixture was stirred at room temperature for overnight. The resulting mixture was concentrated under reduced pressure. The residue was purified by silica gel column chromatography, eluted with PE/EA (3:1) to afford tert-butyl (2S,4S)-2-(hydroxymethyl)-4-(trifluoromethyl)pyrrolidine-1-carboxylate (500 mg, 105.1% yield, 95% purity) as a light yellow oil. MS: m/z: Calc'd for C₁₁H₁₈F₃NO₃ [M+H−56]⁺214. Found, 214.

Step 2: To a stirred solution of tert-butyl (2S,4S)-2-(hydroxymethyl)-4-(trifluoromethyl)pyrrolidine-1-carboxylate (500 mg, 1.85 mmol, 1 eq.) and methanesulfonyl chloride (425.3 mg, 3.71 mmol, 2.00 eq.) in DCM (8 mL) were added TEA (563.7 mg, 5.57 mmol, 3 eq.) at 0° C. The resulting mixture was stirred at room temperature for 2 h. The resulting mixture was concentrated under reduced pressure. The residue was purified by silica gel column chromatography, eluted with PE/EA (3:1) to afford tert-butyl (2S,4S)-2-[(methanesulfonyloxy)methyl]-4-(trifluoromethyl)pyrrolidine-1-carboxylate (650 mg, 100.7% yield, 95% purity) as a light yellow oil. MS: m/z: Calc'd for C₁₂H₂₀F₃NO₅S [M+H−56]⁺292. Found, 292.

Step 3: To a stirred solution of tert-butyl (2S,4S)-2-[(methanesulfonyloxy)methyl]-4-(trifluoromethyl)pyrrolidine-1-carboxylate (400 mg, 1.15 mmol, 1 eq.) and KCN (224.9 mg, 3.45 mmol, 3 eq.) in DMSO (5 mL) at room temperature. The resulting mixture was stirred at 80° C. for overnight. The resulting mixture was extracted with EtOAc (3×100 mL). The combined organic layers were washed with brine (2×50 mL), dried over anhydrous Na₂SO₄. After filtration, the filtrate was concentrated under reduced pressure to afford tert-butyl (2S,4S)-2-(cyanomethyl)-4-(trifluoromethyl)pyrrolidine-1-carboxylate (160 mg, 49.9% yield) as a white solid. MS: m/z: Calc'd for C₁₂H₁₇F₃N₂O₂[M+H−56]⁺223. Found, 223.

Step 4: To a stirred solution of tert-butyl (2S,4S)-2-(cyanomethyl)-4-(trifluoromethyl)pyrrolidine-1-carboxylate (150 mg, 0.539 mmol, 1 equiv) and raney nickel (158.1 mg, 2.69 mmol, 5 eq.) in methanol (4 mL) were added Ammonium hydroxide (9% in water) (18.8 mg, 0.53 mmol, 1 eq.) at room temperature. The resulting mixture was stirred at room temperature under hydrogen atmosphere for 2 h. The precipitated solids were collected by filtration and washed with methanol (4 mL) (2×10 mL). The resulting mixture was concentrated under reduced pressure to afford tert-butyl (2S,4S)-2-(2-aminoethyl)-4-(trifluoromethyl)pyrrolidine-1-carboxylate (75 mg, 49.2% yield) as a light yellow solid. MS: m/z: Calc'd for C₁₂H₂₁F₃N₂O₂[M+H]⁺283. Found, 283.

### Step 5 and 6

The title compound was prepared in 32.8% overall yield as a grey solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S,4S)-2-(2-aminoethyl)-4-(trifluoromethyl)pyrrolidine-1-carboxylate (91-5) in STEP 5; Boc Deprotection; General Procedure II in STEP 6. MS: m/z Calc'd for C₂₂H₂₇F₃N₄O₄[M+H]⁺469. found 469. ¹H NMR (400 MHz, Methanol-d₄) δ 8.27 (s, 1H), 7.76 (d, J=7.9 Hz, 2H), 7.54 (s, 1H), 7.45 (dd, J=8.4, 2.8 Hz, 2H), 5.03 (s, 1H), 4.41 (s, 1H), 4.28 (s, 1H), 3.75-3.26 (m, 7H), 3.23-3.20 (m, 2H), 3.10 (s, 1H), 2.65-2.62 (m, 1H), 2.08-1.84 (d, J=11.4 Hz, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 27% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-{2-[(3S)-pyrrolidin-3-yl]ethyl}carbamate (V193011)

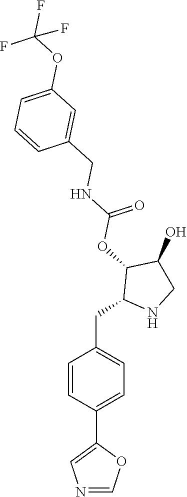 [SMILES: O=C(NCc1cccc(OC(F)(F)F)c1)O[C@@H]1[C@@H](O)CN[C@@H]1Cc1ccc(-c2cnco2)cc1]

The title compound was prepared in 25.1% overall yield as an off-white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (3R)-3-(2-aminoethyl)pyrrolidine-1-carboxylate in STEP 1; Boc Deprotection; General Procedure II in STEP 2. MS: m/z Calc'd for C₂₁H₂₈N₄O₄ [M+H]⁺401. found 401. ¹H NMR (400 MHz, Methanol-d₄) δ 8.28 (s, 1H), 7.76 (d, J=8.2 Hz, 2H), 7.54 (s, 1H), 7.45 (d, J=8.2 Hz, 2H), 5.00 (d, J=3.6 Hz, 1H), 4.40 (d, J=4.1 Hz, 1H), 4.27 (s, 1H), 3.62-3.50 (m, 1H), 3.50-3.36 (m, 1H), 3.26 (d, J=6.3 Hz, 1H), 3.26-3.18 (m, 5H), 3.09 (dd, J=14.1, 8.6 Hz, 1H), 2.87 (dd, J=11.5, 9.1 Hz, 1H), 2.34 (dd, J=16.7, 7.9 Hz, 1H), 2.28 (s, 1H), 1.79-1.61 (m, 3H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-{2-[(2R)-4-fluoropyrrolidin-2-yl]ethyl}carbamate (V192916)

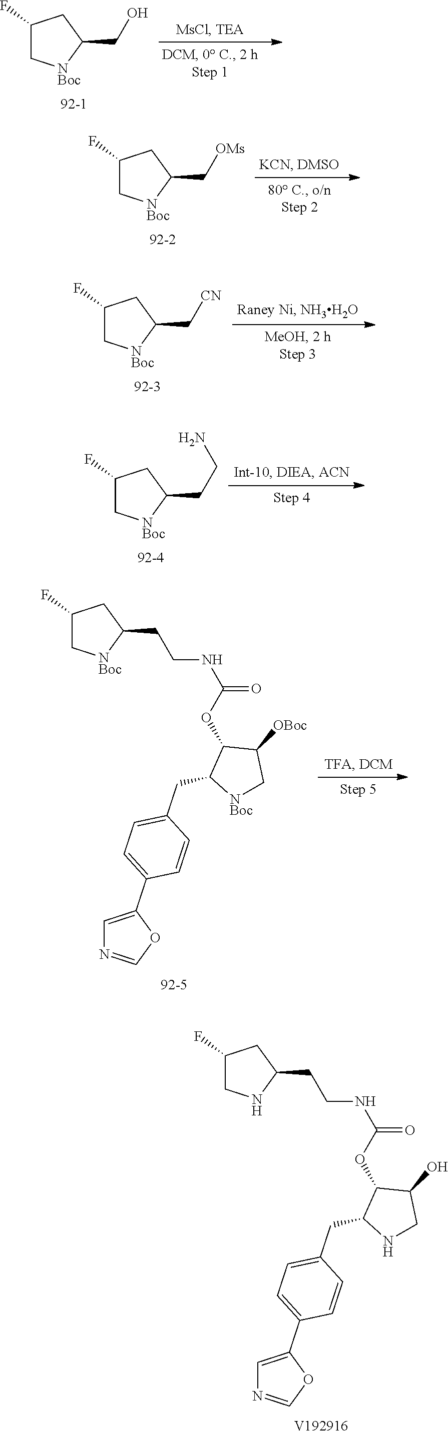

Step 1: To a stirred solution of tert-butyl (2S)-4-fluoro-2-(hydroxymethyl)pyrrolidine-1-carboxylate (500 mg, 2.28 mmol, 1 eq.) and methanesulfonyl chloride (522.4 mg, 4.56 mmol, 2 eq.) in DCM (8 mL) were added TEA (692.2 mg, 6.84 mmol, 3 eq.) at 0° C. The resulting mixture was stirred at room temperature for 2 h. The resulting mixture was concentrated under reduced pressure. The residue was purified by silica gel column chromatography, eluted with PE/EA (2:1) to afford tert-butyl (2S)-4-fluoro-2-[(methanesulfonyloxy)methyl]pyrrolidine-1-carboxylate (600 mg, 88.4% yield, 95% purity) as a light yellow oil. MS: m/z: Calc'd for C₁₁H₂₀FNO₅S [M+H−56]⁺242. Found, 242.

Step 2: To a stirred solution of tert-butyl (2S)-4-fluoro-2-[(methanesulfonyloxy)methyl]pyrrolidine-1-carboxylate (600 mg, 2.01 mmol, 1 eq.) in DMSO (10 mL) were added KCN (262.7 mg, 4.03 mmol, 2 eq.) at 0° C. The resulting mixture was stirred at room temperature for overnight. The resulting mixture was extracted with EtOAc (2×300 mL). The combined organic layers were washed with brine (2×100 mL), dried over anhydrous Na₂SO₄. After filtration, the filtrate was concentrated under reduced pressure to afford tert-butyl (2R)-2-(cyanomethyl)-4-fluoropyrrolidine-1-carboxylate (350 mg, 75.9% yield, 80% purity) as a light yellow semi-solid. MS: m/z: Calc'd for C₁₁H₁₇FN₂O₂[M+H]⁺229. Found, 229.

Step 3: To a stirred solution of tert-butyl (2R)-2-(cyanomethyl)-4-fluoropyrrolidine-1-carboxylate (345 mg, 1.51 mmol, 1 eq.) and raney nickel (266.1 mg, 4.53 mmol, 3 eq.) in methanol (5 mL) were added Ammonium hydroxide (9% in water) (52.9 mg, 1.51 mmol, 1 eq.) at room temperature under hydrogen atmosphere. The resulting mixture was stirred at room temperature for 2 h. The resulting mixture was filtered, the filter cake was washed with methanol (5 mL). The filtrate was concentrated under reduced pressure to afford tert-butyl (2R)-2-(2-aminoethyl)-4-fluoropyrrolidine-1-carboxylate (250 mg) as a light yellow semi-solid. MS: m/z: Calc'd for C₁₁H₂₁FN₂O₂[M+H]⁺233. Found, 233.

### Step 4 and 5

The title compound was prepared in 24.8% overall yield as an off-white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2R)-2-(2-aminoethyl)-4-fluoropyrrolidine-1-carboxylate (92-4) in STEP 4; Boc Deprotection; General Procedure II in STEP 5. MS: m/z Calc'd for C₂₁H₂₇FN₄O₄ [M+H]⁺419. found 419. ¹H NMR (400 MHz, Methanol-d₄) δ 8.27 (s, 1H), 7.76 (d, J=7.9 Hz, 2H), 7.54 (s, 1H), 7.45 (dd, J=8.4, 2.8 Hz, 2H), 5.53-5.46 (m, 1H), 5.03 (s, 1H), 4.41 (s, 1H), 4.28 (s, 1H), 3.85 (s, 1H), 3.75 (s, 1H), 3.70-3.46 (m, 2H), 3.23 (dd, J=15.6, 9.3 Hz, 3H), 3.10 (s, 1H), 3.05 (dd, J=13.5, 7.2 Hz, 1H), 2.78 (s, 1H), 2.08 (s, 3H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-{2-[(6R)-5-azaspiro[2.4]heptan-6-yl]ethyl}carbamate (V193018)

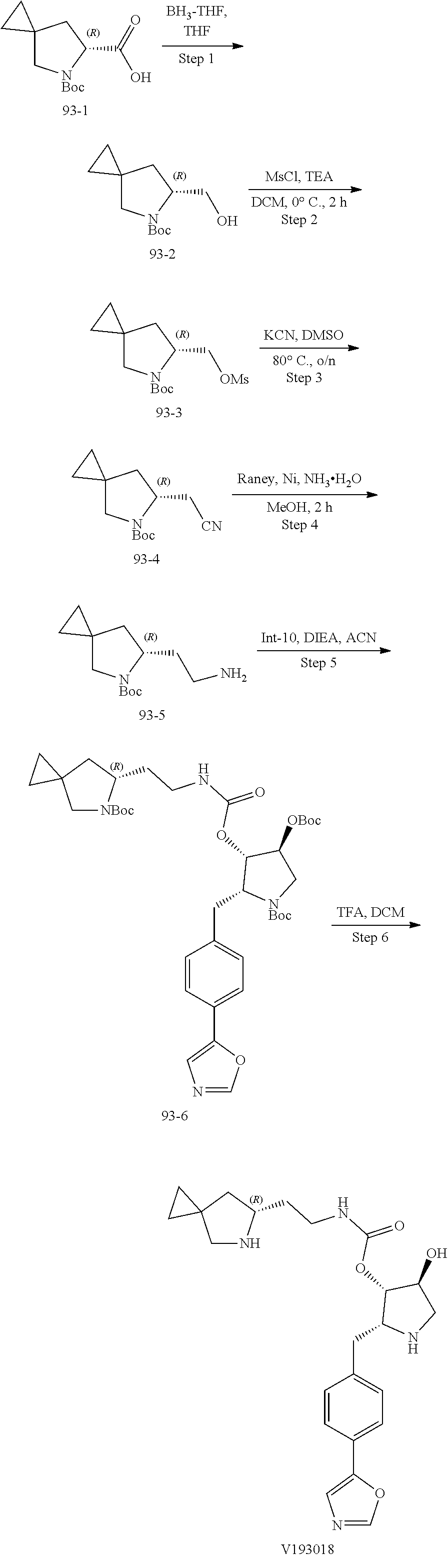

Step 1: To a stirred solution of (6R)-5-(tert-butoxycarbonyl)-5-azaspiro[2.4]heptane-6-carboxylic acid (1 g, 4.14 mmol, 1 eq.) in THF (6 mL) were added Borane-tetrahydrofuran complex (1.0 M in THF) (6 mL) at 0° C. The resulting mixture was stirred at room temperature for overnight. The resulting mixture was concentrated under reduced pressure. The residue was purified by silica gel column chromatography, eluted with PE/EA (3:1) to afford tert-butyl (6R)-6-(hydroxymethyl)-5-azaspiro[2.4]heptane-5-carboxylate (930 mg, 98.7% yield, 95% purity) as a light yellow oil. MS: m/z: Calc'd for C₁₂H₂₁NO₃ [M+H−56]⁺172. Found, 172.

Step 2: To a stirred solution of tert-butyl (6R)-6-(hydroxymethyl)-5-azaspiro[2.4]heptane-5-carboxylate (500 mg, 2.20 mmol, 1 eq.) and methanesulfonyl chloride (503.9 mg, 4.40 mmol, 2 eq.) in DCM (8 mL) were added TEA (667.7 mg, 6.60 mmol, 3 eq.) at 0° C. The resulting mixture was stirred at room temperature for 2 h. The resulting mixture was concentrated under reduced pressure. The residue was purified by silica gel column chromatography, eluted with PE/EA (3:1) to afford tert-butyl (6R)-6-[(methanesulfonyloxy)methyl]-5-azaspiro[2.4]heptane-5-carboxylate (600 mg, 89.32% yield) as a light yellow oil. MS: m/z: Calc'd for C₁₃H₂₃NO₅S [M+H−56]⁺250. Found, 250.

Step 3: To a stirred solution of tert-butyl (6R)-6-[(methanesulfonyloxy)methyl]-5-azaspiro[2.4]heptane-5-carboxylate (400 mg, 1.31 mmol, 1 eq.) and KCN (255.8 mg, 3.93 mmol, 3 eq.) in DMSO (5 mL) at room temperature. The resulting mixture was stirred at 80° C. for overnight. The resulting mixture was extracted with EtOAc (3×100 mL). The combined organic layers were washed with brine (2×50 mL), dried over anhydrous Na₂SO₄. After filtration, the filtrate was concentrated under reduced pressure to afford tert-butyl (6R)-6-(cyanomethyl)-5-azaspiro[2.4]heptane-5-carboxylate (250 mg, 80.7% yield, 80% purity) as a white solid. MS: m/z: Calc'd for C₁₃H₂₀N₂O₂ [M+H−56]⁺181. Found, 181.

Step 4: To a stirred solution of tert-butyl (6R)-6-(cyanomethyl)-5-azaspiro[2.4]heptane-5-carboxylate (245 mg, 1.03 mmol, 1 eq.) and raney nickel (182.5 mg, 3.11 mmol, 3 eq.) in methanol (5 mL) were added Ammonium hydroxide (9% in water) (36.3 mg, 1.03 mmol, 1 eq.) at room temperature. The resulting mixture was stirred at room temperature under hydrogen atmosphere for 2 h. The precipitated solids were collected by filtration and washed with methanol (4 mL) (2×10 mL). The resulting mixture was concentrated under reduced pressure to afford tert-butyl (6R)-6-(2-aminoethyl)-5-azaspiro[2.4]heptane-5-carboxylate as a light yellow oil. MS: m/z: Calc'd for C₁₃H₂₄N₂O₂ [M+H]⁺241. Found, 241.

### Step 5 and 6

The title compound was prepared in 26.8% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (6R)-6-(2-aminoethyl)-5-azaspiro[2.4]heptane-5-carboxylate (93-5) in STEP 4; Boc Deprotection; General Procedure II in STEP 5. MS: m/z Calc'd for C₂₃H₃₀N₄O₄ [M+H]⁺427. found 427. ¹H NMR (400 MHz, Methanol-d₄) δ 8.28 (s, 1H), 7.76 (d, J=8.2 Hz, 2H), 7.54 (s, 1H), 7.45 (d, J=8.0 Hz, 2H), 5.05-5.00 (m, 1H), 4.41 (d, J=4.1 Hz, 1H), 4.27 (s, 1H), 3.84 (p, J=7.6 Hz, 1H), 3.60 (dd, J=12.6, 4.2 Hz, 1H), 3.32 (d, J=15.3 Hz, 6H), 3.18-3.05 (m, 1H), 2.16-1.89 (m, 4H), 0.88-0.69 (m, 4H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 27% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-{2-[(2S)-1-benzyl-4-(oxolan-2-ylmethyl)piperazin-2-yl]ethyl}carbamate (V187345)

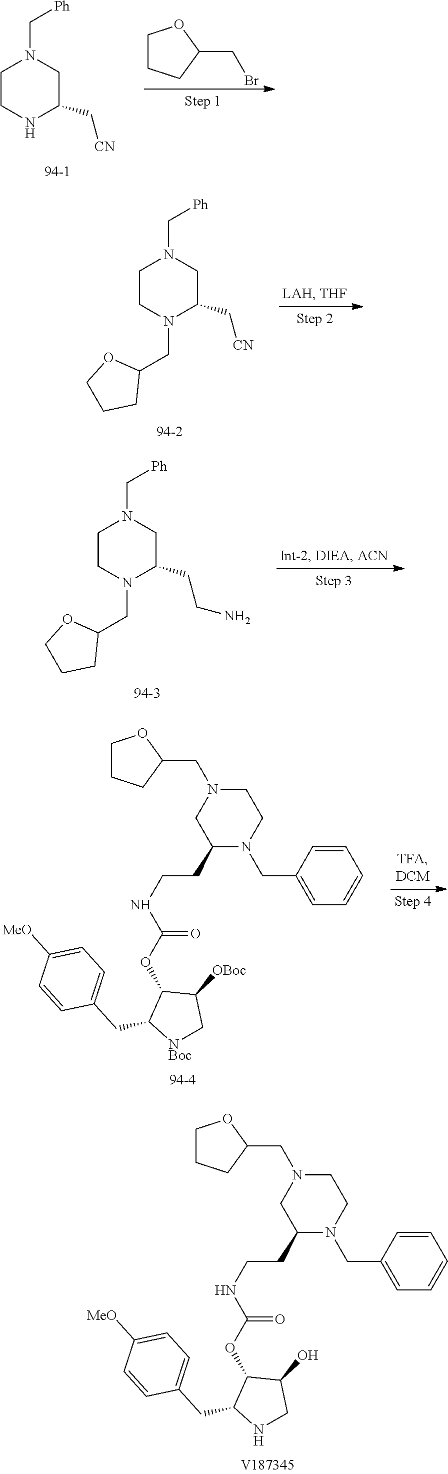

Step 1: To a stirred solution of 2-[(2S)-4-benzylpiperazin-2-yl]acetonitrile (400 mg, 1.858 mmol, 1 equiv) and 2-(bromomethyl)oxolane (306.6 mg, 1.86 mmol, 1 eq.) in DMF (5 ml) were added K₂CO₃ (513.5 mg, 3.72 mmol, 2 eq.) and KI (30.8 mg, 0.19 mmol, 0.1 eq.) in portions at room temperature under air atmosphere. The final reaction mixture was irradiated with microwave radiation for 2 h at 80° C. After completion of reaction monitored by LCMS. The residue was purified by reversed-phase flash chromatography to afford 2-[(2S)-4-benzyl-1-(oxolan-2-ylmethyl)piperazin-2-yl]acetonitrile (222 mg, 35.9%) as a white solid. MS: m/z: Calc'd for C₁₈H₂₅N₃O [M+H]⁺ 300. found 300.

Step 2: To a stirred solution of 2-[(2S)-4-benzyl-1-(oxolan-2-ylmethyl)piperazin-2-yl]acetonitrile (200 mg, 0.67 mmol, 1 eq.) in THF (5 ml) was added LAH (1 mL) dropwise at 0° C. under air atmosphere. The final reaction mixture was irradiated with microwave radiation for 2 h at 0° C. After completion of reaction monitored by LCMS. The reaction was quenched with MeOH at 0° C. The resulting mixture was concentrated under reduced pressure to afford 2-[(2S)-4-benzyl-1-(oxolan-2-ylmethyl)piperazin-2-yl]ethanamine (150 mg, 74%) as a white solid. MS: m/z: Calc'd for C₁₈H₂₉N₃O [M+H]⁺ 304. found 304.

Step 3 and 4: The title compound was prepared in 10.5% overall yield as a white solid according to Substitution reaction for synthesis of Carbamate; General Procedure I using 2-[(2S)-4-benzyl-1-(oxolan-2-ylmethyl)piperazin-2-yl]ethanamine (94-3) in STEP 3; Boc Deprotection; General Procedure II in STEP 4. MS: m/z Calc'd for C₃₁H₄₄N₄O₅ [M+H]⁺553. found 553. ¹H NMR (400 MHz, Methanol-d₄) δ 7.56-7.35 (m, 5H), 7.27-7.20 (m, 2H), 6.99-6.85 (m, 2H), 5.04-4.95 (m, 1H), 4.37 (t, J=4.7 Hz, 1H), 4.27-4.12 (m, 2H), 3.99-3.86 (m, 2H), 3.85-3.70 (m, 5H), 3.56 (dd, J=12.6, 4.3 Hz, 2H), 3.36 (s, 2H), 3.30-3.18 (m, 4H), 3.17-3.02 (m, 3H), 2.96 (dd, J=14.2, 8.7 Hz, 2H), 2.86 (d, J=19.7 Hz, 2H), 2.23-1.86 (m, 5H), 1.69-1.56 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 29% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-[2-(1H-pyrazol-4-yl)ethyl]carbamate (V187509)

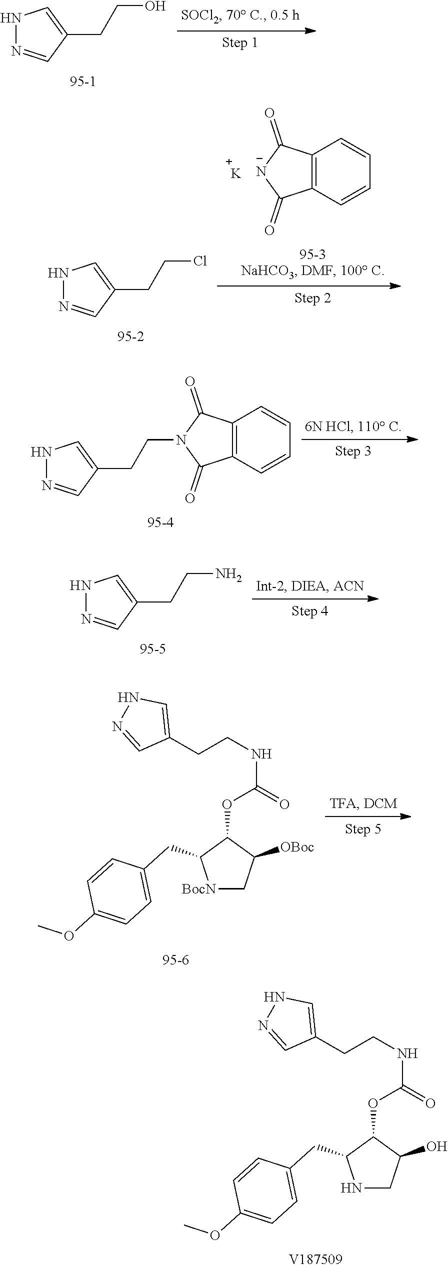

Step 1: To a stirred solution of 2-(1H-pyrazol-4-yl)ethanol (1 g, 8.92 mmol, 1 eq.) was added SOCl₂ (5 mL) in portions at room temperature under air atmosphere. The final reaction mixture was irradiated with microwave radiation for 0.5 h at 70° C. The resulting mixture was concentrated under reduced pressure. After completion of reaction monitored by LCMS. This resulted in 4-(2-chloroethyl)-1H-pyrazole (1.2 g, 92.7%) as a yellow solid. The crude product mixture was used in the next step directly without further purification. MS: m/z: Calc'd for C₅H₇ClN₂ [M+H]⁺131. found 131.

Step 2: To a stirred solution of 4-(2-chloroethyl)-1H-pyrazole (400 mg, 3.06 mmol, 1 eq.) and potassium phthalimide (680.9 mg, 3.68 mmol, 1.2 eq.) in DMF (5 ml) was added NaHCO₃ (220.5 mg, 9.19 mmol, 3 eq.) in portions at room temperature under air atmosphere. The final reaction mixture was irradiated with microwave radiation for 2 h at 100° C. After completion of reaction monitored by LCMS. The residue was purified by reversed-phase flash chromatography to afford 2-[2-(1H-pyrazol-4-yl)ethyl]isoindole-1,3-dione (400 mg, 48.7%) as a white solid. MS: m/z: Calc'd for C₁₃H₁₁N₃O₂ [M+H]⁺242. found 242.

Step 3: A solution of 2-[2-(1H-pyrazol-4-yl)ethyl]isoindole-1,3-dione (400 mg, 1.66 mmol, 1 eq.) and HCl (6M) (6 mL) was stirred for overnight at 110° C. under air atmosphere. After completion of reaction monitored by LCMS. The resulting mixture was concentrated under reduced pressure. This resulted in 2-(1H-pyrazol-4-yl)ethanamine (350 mg, 170.9%) as a white solid. The crude product mixture was used in the next step directly without further purification. MS: m/z: Calc'd for C₅H₉N₃ [M+H]⁺ 112. found 112.

### Step 4 and 5

The title compound was prepared in 13.9% overall yield as a white solid according to Substitution reaction for synthesis of Carbamate; General Procedure I using 2-(1H-pyrazol-4-yl)ethanamine (95-5) in STEP 4; Boc Deprotection; General Procedure II in STEP 5. MS: m/z Calc'd for C₁₈H₂₄N₄O₄ [M+H]⁺361. found 361. ¹H NMR (400 MHz, Methanol-d₄) δ 7.56 (d, J=2.9 Hz, 2H), 7.28-7.14 (m, 2H), 6.97-6.87 (m, 2H), 4.92 (d, J=3.6 Hz, 1H), 4.35 (d, J=4.2 Hz, 1H), 4.19-4.07 (m, 1H), 3.79 (s, 3H), 3.54 (dd, J=12.6, 4.3 Hz, 1H), 3.46-3.36 (m, 2H), 3.20 (d, J=12.7 Hz, 1H), 3.08 (dd, J=14.1, 7.2 Hz, 1H), 2.98-2.91 (m, 1H), 2.76 (t, J=7.0 Hz, 2H).

Prep-HPLC purification conditions: Column: Xselect CSH Prep C18 C18 Column, 30*150 mm, 5 μm; Mobile Phase A: Water (0.05% TFA), Mobile Phase B: ACN; Flow rate: 60 mL/min; Gradient: 2% B to 24% B in 10 min; Wave Length: 254 nm/220 nm; RT1(min): 8.68.

### Synthetic Procedure of (2R,3S,4S)-4-hydroxy-2-[(4-methoxyphenyl)methyl]pyrrolidin-3-yl N-[2-(1H-pyrazol-3-ylamino)ethyl]carbamate (V187827)

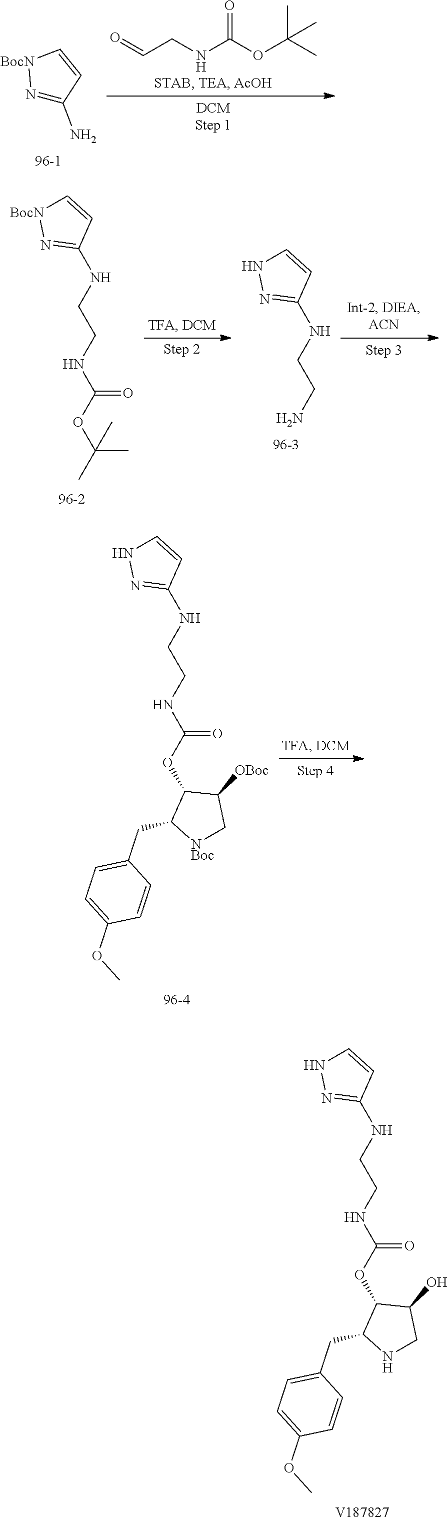

Step 1: A solution of tert-butyl 3-aminopyrazole-1-carboxylate (100 mg, 0.55 mmol, 1 eq.) and tert-butyl N-(2-oxoethyl)carbamate (173.8 mg, 1.09 mmol, 2 eq.) in EtOH (10 mL) was treated with ZnCl₂ (223.2 mg, 1.64 mmol, 3 eq.) for 1 h at 80° C. under nitrogen atmosphere followed by the addition of NaBH₃CN (68.6 mg, 1.09 mmol, 2 eq.) at 0° C. The resulting mixture was stirred for 1 h at room temperature. After completion of reaction monitored by LCMS. The reaction was quenched with Water at 0° C. The resulting mixture was concentrated under reduced pressure. The residue was purified by flash. This resulted in tert-butyl N-[2-(1H-pyrazol-3-ylamino)ethyl]carbamate (60 mg, 48.6%) as a light yellow oil. MS: m/z: Calc'd for C₁₀H₁₈N₄O₂[M+H]⁺ 227. found 227.

Step 2: To a stirred solution of tert-butyl N-[2-(1H-pyrazol-3-ylamino)ethyl]carbamate (50 mg, 0.22 mmol, 1 eq.) in DCM (5 mL) was added TFA (1 mL) at 0° C. The resulting mixture was stirred at room temperature for 1 h. After completion of reaction monitored by LCMS. The crude product was used in the next step directly without further purification. MS: m/z: Calc'd for C₅H₁₀N₄ [M+H]⁺127. found 127.

Step 3 and 4: The title compound was prepared in 12.4% overall yield as a grey solid according to Substitution reaction for synthesis of Carbamate; General Procedure I using N1-(1H-pyrazol-3-yl)ethane-1,2-diamine (96-3) in STEP 3; Boc Deprotection; General Procedure II in STEP 4. MS: m/z Calc'd for C₁₈H₂₅N₅O₄ [M+H]⁺376. found 376. ¹H NMR (400 MHz, Methanol-d₄) δ 7.68 (d, J=17.4 Hz, 1H), 7.22 (d, J=8.5 Hz, 2H), 6.96-6.87 (m, 2H), 4.96 (d, J=3.7 Hz, 2H), 4.37 (d, J=4.2 Hz, 1H), 4.20-4.11 (m, 1H), 3.79 (d, J=3.7 Hz, 3H), 3.55 (dd, J=12.6, 4.2 Hz, 1H), 3.32-3.29 (m, 4H), 3.21 (d, J=12.6 Hz, 1H), 3.10 (dd, J=14.2, 7.0 Hz, 1H), 2.94 (dd, J=14.2, 8.6 Hz, 1H).

Prep-HPLC purification conditions: Column: Xbridge Prep OBD C18 Column, 19*250 mm, 5 μm; Mobile Phase A: Water (10 mmol/L NH₄HCO₃), Mobile Phase B: ACN; Flow rate: 25 mL/min; Gradient: 10% B to 20% B in 12 min; Wave Length: 254 nm/220 nm; RT1(min): 9.72.

### Synthetic Procedure of (2R,3S,4S)-4-hydroxy-2-[(4-methoxyphenyl)methyl]pyrrolidin-3-yl N-[2-(1H-pyrazol-3-ylamino)ethyl]carbamate (V187944)

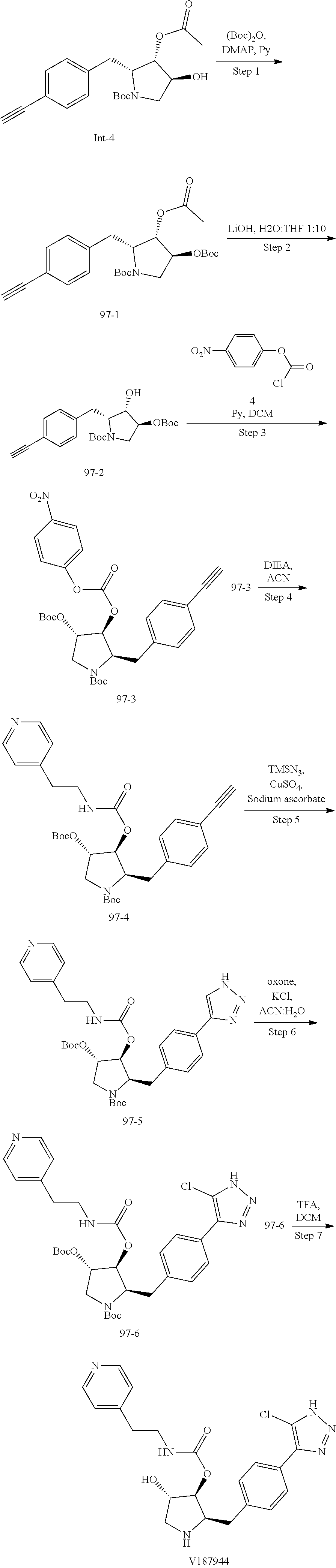

Step 1: To a stirred solution of tert-butyl (2R,3S,4S)-3-(acetyloxy)-2-[(4-ethynylphenyl)methyl]-4-hydroxypyrrolidine-1-carboxylate (Int-4, 500 mg, 1.39 mmol, 1 eq.) and DMAP (254.9 mg, 2.09 mmol, 1.5 eq.) in Pyridine (10 mL) was added Boc₂O (1.5 g, 6.95 mmol, 5 eq.) at room temperature. The resulting mixture was stirred at room temperature for 12 h. After completion of reaction monitored by LCMS. The residue was purified by flash. This resulted in tert-butyl (2R,3S,4S)-3-(acetyloxy)-4-[(tert-butoxycarbonyl)oxy]-2-[(4-ethynylphenyl)methyl]pyrrolidine-1-carboxylate (625 mg, 97.8%) as a light yellow oil. MS: m/z: Calc'd for C₂₅H₃₃NO₇ [M+H+22+41]⁺523. found 523.

Step 2: The compound was prepared in 65.2% yield as a white solid according to Hydrolysis reaction with LiOH; General Procedure V; MS: m/z: Calc'd for C₂₃H₃₁NO₆ [M+H+22+41]⁺481, found 481.

Step 3: To a stirred solution of tert-butyl (2R,3S,4S)-4-[(tert-butoxycarbonyl)oxy]-2-[(4-ethynylphenyl)methyl]-3-hydroxypyrrolidine-1-carboxylate (97-2, 360 mg, 0.86 mmol, 1 eq.) and 4-nitrophenyl carbonochloridate (260.7 mg, 1.29 mmol, 1.5 eq.) in DCM (10 mL) was added Pyridine (136.4 mg, 1.72 mmol, 2 eq.) at room temperature. The resulting mixture was stirred for 4 h at room temperature under nitrogen atmosphere. After completion of reaction monitored by LCMS. The residue was purified by Prep-TLC (PE:EA, 4:1). This resulted in tert-butyl (2R,3S,4S)-4-[(tert-butoxycarbonyl)oxy]-2-[(4-ethynylphenyl)methyl]-3-[(4-nitrophenoxycarbonyl)oxy]pyrrolidine-1-carboxylate (470 mg, 93.6%) as a colorless oil. MS: m/z: Calc'd for C₃₀H₃₄N₂O₁₀ [M+H−100]⁺483, found 483.

Step 4: The compound was prepared in 85% yield as a light yellow oil according to Substitution reaction for synthesis of Carbamate; General Procedure I using 4-pyridine etheneamine; MS: m/z: Calc'd for C₃₁H₃₉N₃O₇ [M+H]⁺566. found 566.

Step 5: To a stirred solution of tert-butyl (2R,3S,4S)-4-[(tert-butoxycarbonyl)oxy]-2-[(4-ethynylphenyl)methyl]-3-({[2-(pyridin-4-yl)ethyl]carbamoyl}oxy)pyrrolidine-1-carboxylate (200 mg, 0.35 mmol, 1 eq.) and Azidotrimethylsilane (81.5 mg, 0.71 mmol, 2 eq.) in MeOH (20 mL) were added CuSO₄·5H₂O (22.5 mg, 0.35 mmol, 1 eq.) and sodium (5R)-5-[(1S)-1,2-dihydroxyethyl]-3,4-dihydroxy-2,5-dihydrofuran-2-one (70.4 mg, 0.35 mmol, 1 eq.) at 0° C. The resulting mixture was stirred at room temperature for 4 h under nitrogen atmosphere. After completion of reaction monitored by LCMS. The residue was purified by flash. This resulted in tert-butyl (2R,3S,4S)-4-[(tert-butoxycarbonyl)oxy]-3-({[2-(pyridin-4-yl)ethyl]carbamoyl}oxy)-2-{[4-(1H−1,2,3-triazol-4-yl)phenyl]methyl}pyrrolidine-1-carboxylate (120 mg, 55.8%) as a light yellow oil. MS: m/z: Calc'd for C₃₁H₄₀N₆O₇[M+H]⁺609. found 609.

Step 6: To a stirred solution of tert-butyl (2R,3S,4S)-4-[(tert-butoxycarbonyl)oxy]-3-({[2-(pyridin-4-yl)ethyl]carbamoyl}oxy)-2-{[4-(1H−1,2,3-triazol-4-yl)phenyl]methyl}pyrrolidine-1-carboxylate (105 mg, 0.17 mmol, 1 eq.) in DMF (5 mL) were added NCS (35.7 mg, 0.22 mmol, 1.3 eq.) at 50° C. under nitrogen atmosphere. The resulting mixture was stirred at room temperature for 12h under nitrogen atmosphere. After completion of reaction monitored by LCMS. The residue was purified by flash. This resulted in tert-butyl (2R,3S,4S)-4-[(tert-butoxycarbonyl)oxy]-2-{[4-(5-chloro-1H−1,2,3-triazol-4-yl)phenyl]methyl}-3-({[2-(pyridin-4-yl)ethyl]carbamoyl}oxy)pyrrolidine-1-carboxylate (28 mg, 25.2%) as a white solid. MS: m/z: Calc'd for C₃₁H₃₉ClN₆O₇[M+H]⁺ 643. found 643.

Step 7: The title compound was prepared in 42.9% yield as a light yellow solid according to Boc Deprotection; General Procedure II. MS: m/z Calc'd for C₂₁H₂₃ClN₆O₃[M+H]⁺443. found 443. ¹H NMR (400 MHz, Methanol-d₄) δ 8.73-8.61 (m, 2H), 7.93 (d, J=7.9 Hz, 2H), 7.82 (d, J=5.6 Hz, 2H), 7.45 (d, J=8.0 Hz, 2H), 4.99 (d, J=3.5 Hz, 1H), 4.37 (d, J=4.0 Hz, 1H), 4.29-4.24 (m, 1H), 3.63-3.51 (m, 3H), 3.23 (dd, J=13.4, 9.8 Hz, 2H), 3.15-3.00 (m, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 24% B in 10 min; Wave Length: 254 nm/220 nm; RT1(min): 9.02.

### Synthetic Procedure of (2S)-2-{2-[({[(2R,3S,4S)-4-hydroxy-2-[(4-methoxyphenyl)methyl]pyrrolidin-3-yl]oxy}carbonyl)amino]acetamido}propanoic acid (V188019)

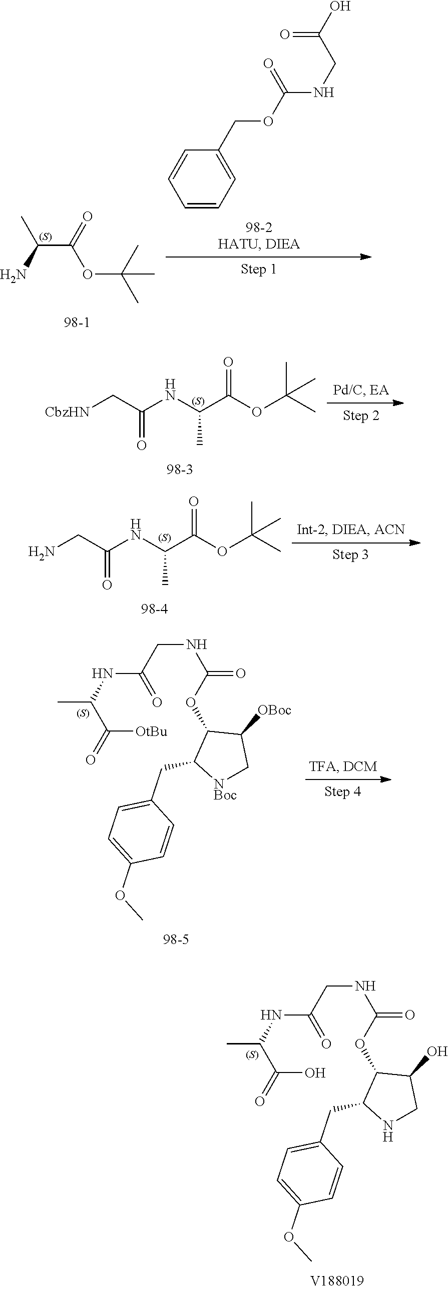

Step 1: To a stirred solution of tert-butyl (2S)-2-aminopropanoate (300 mg, 2.07 mmol, 1 eq.) and (cbz)gly (432.2 mg, 2.07 mmol, 1 eq.) in DMF (5 mL) were added HATU (1.6 g, 4.13 mmol, 2 eq.) and DIEA (1.1 g, 8.26 mmol, 4 eq.) at room temperature. The resulting mixture was stirred at room temperature for 4 h under nitrogen atmosphere. After completion of reaction monitored by LCMS. After completion of reaction monitored by LCMS. This resulted in tert-butyl (2S)-2-(2-{[(benzyloxy)carbonyl]amino}acetamido)propanoate (600 mg, 86.3%) as a white solid. MS: m/z: Calc'd for C₁₇H₂₄N₂O₅ [M+H+22]⁺359. found 359.

Step 2: To a stirred solution of tert-butyl (2S)-2-(2-{[(benzyloxy)carbonyl]amino}acetamido)propanoate (300 mg, 0.89 mmol, 1 eq.) in EA (25 mL) was added Pd/C (99.7 mg, 0.94 mmol, 1.1 eq.) at room temperature under nitrogen atmosphere. The resulting mixture was stirred at room temperature for 4 h under hydrogen atmosphere. After completion of reaction monitored by LCMS. The crude product was used in the next step directly without further purification. MS: m/z: Calc'd for C₉H₁₈N₂O₃[M+H−56]⁺ 147. found 147.

Step 3 and 4: The title compound was prepared in 12.4% overall yield as a white solid according to Substitution reaction for synthesis of Carbamate; General Procedure I using tert-butyl (2S)-2-(2-aminoacetamido)propanoate (98-4) in STEP 3; Boc Deprotection; General Procedure II in STEP 4. MS: m/z Calc'd for C₁₈H₂₅N₃O₇ [M+H]⁺396. found 396. ¹H NMR (400 MHz, Methanol-d₄) δ 7.30-7.22 (m, 2H), 6.97-6.91 (m, 2H), 4.97 (d, J=3.3 Hz, 1H), 4.44 (d, J=7.3 Hz, 2H), 4.20-4.11 (m, 1H), 3.89 (s, 2H), 3.80 (s, 3H), 3.61 (dd, J=12.6, 4.5 Hz, 1H), 3.52-3.38 (m, 1H), 3.21 (d, J=12.6 Hz, 1H), 3.13 (d, J=6.8 Hz, 1H), 2.97 (dd, J=14.2, 8.7 Hz, 1H), 1.44 (d, J=7.2 Hz, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 26% B in 10 min; Wave Length: 254 nm/220 nm; RT1(min): 8.55.

### Synthetic Procedure of (2R,3S,4S)-4-hydroxy-2-[(4-methoxyphenyl)methyl]pyrrolidin-3-yl N-[(2-aminopyrimidin-5-yl)methyl]carbamate (V188037)

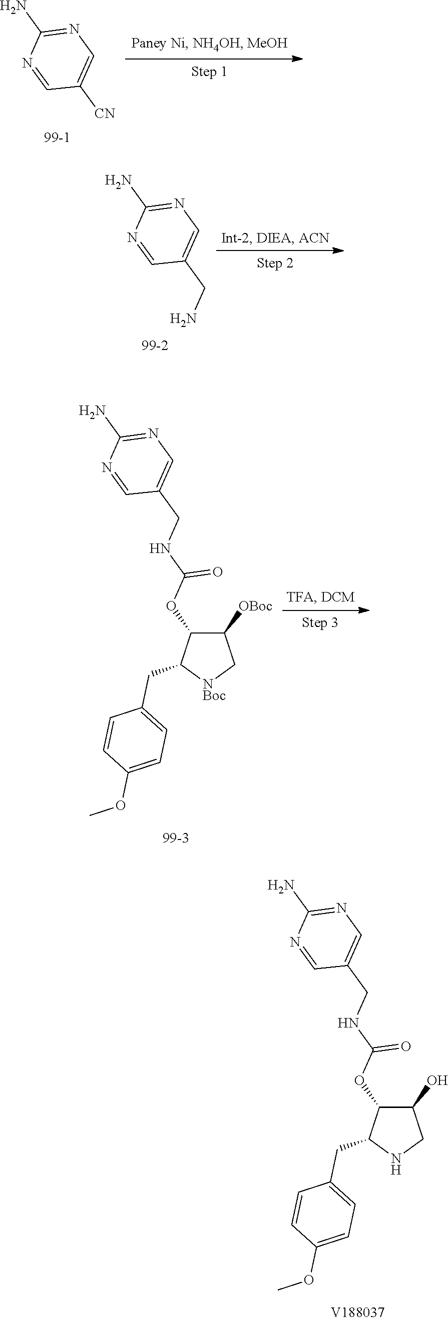

Step 1: To a stirred solution of 2-aminopyrimidine-5-carbonitrile (100 mg, 0.83 mmol, 1 eq.) and NH₄OH (87.5 mg, 2.50 mmol, 3 eq.) in MeOH (10 mL) was added Raney Ni (463.6 mg, 5.41 mmol, 6.5 eq.) at room temperature under nitrogen atmosphere. The resulting mixture was stirred at room temperature for 2 h under hydrogen atmosphere. TLC showed a new point. The crude product was used in the next step directly without further purification.

Step 2 and 3: The title compound was prepared in 8.2% overall yield as a light yellow solid according to Substitution reaction for synthesis of Carbamate; General Procedure I using 5-(aminomethyl)pyrimidin-2-amine (99-2) in STEP 2; Boc Deprotection; General Procedure II in STEP 3. MS: m/z Calc'd for C₁₈H₂₃N₅O₄ [M+H]⁺374. found 374. ¹H NMR (400 MHz, Methanol-d₄) δ 8.29 (s, 2H), 7.09 (d, J=8.5 Hz, 2H), 6.85-6.78 (m, 2H), 4.68-4.58 (m, 3H), 4.24-4.15 (m, 2H), 4.10 (d, J=15.0 Hz, 1H), 3.78 (s, 3H), 3.65 (s, 1H), 2.83 (dd, J=9.2, 4.4 Hz, 2H).

Prep-HPLC purification conditions: Column: XBridge Prep OBD C18 Column, 30*150 mm, 5 μm; Mobile Phase A: Water (10 nmol/LNH₄HCO₃), Mobile Phase B: ACN; Flow rate: 60 mL/min; Gradient: 2% B to 26% B in 10 min; Wave Length: 254 nm/220 nm; RT1(min): 8.67.

### Synthetic Procedure of (2R,3S,4S)-4-hydroxy-2-[(4-methoxyphenyl)methyl]pyrrolidin-3-yl N-{[2-(trifluoromethyl)pyridin-4-yl]methyl}carbamate (V188050)

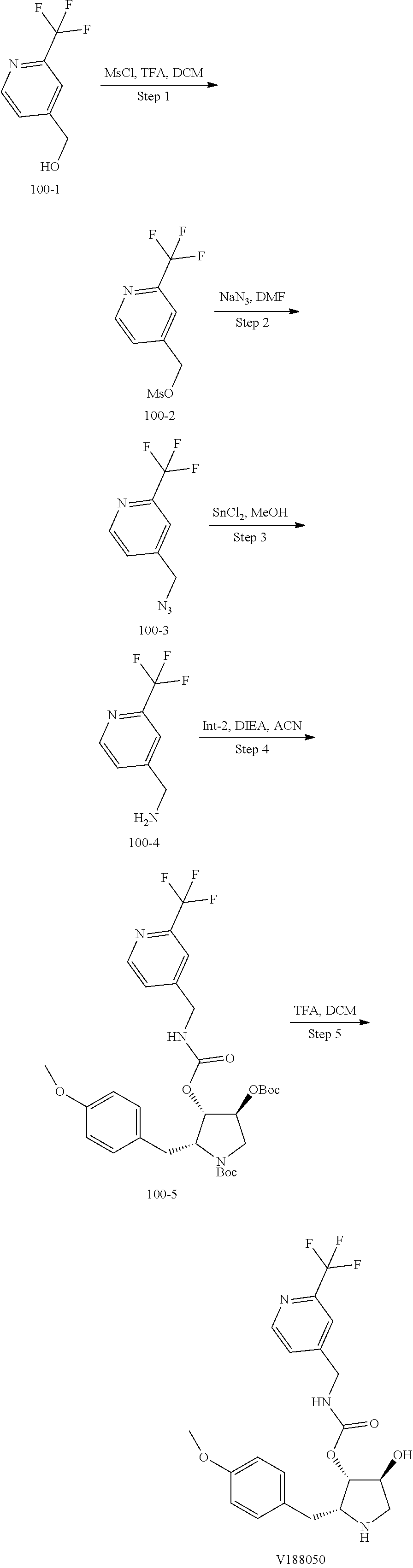

Step 1: To a stirred solution of [2-(trifluoromethyl)pyridin-4-yl]methanol (200 mg, 1.13 mmol, 1 eq.) and TEA (171.4 mg, 1.69 mmol, 1.5 eq.) in DCM (5 mL) was added MsCl (155.2 mg, 1.36 mmol, 1.2 eq.) at 0° C. The resulting mixture was stirred at room temperature for 1 h under nitrogen atmosphere. After completion of reaction monitored by LCMS. The aqueous layer was extracted with DCM. The crude product was used in the next step directly without further purification. MS: m/z: Calc'd for C₈H₈F₃NO₃S [M+H]⁺ 256. found 256.

Step 2: To a stirred solution of [2-(trifluoromethyl)pyridin-4-yl]methyl methanesulfonate (280 mg, 1.09 mmol, 1 eq.) in DMF (6 mL) was added NaN₃ (213.9 mg, 3.29 mmol, 3 eq.) at room temperature. The resulting mixture was stirred at room temperature for 12 h under nitrogen atmosphere. After completion of reaction monitored by LCMS. The aqueous layer was extracted with EA. The crude product was used in the next step directly without further purification. MS: m/z: Calc'd for C₇H₅F₃N₄[M+H]⁺ 203. found 203.

Step 3: To a stirred solution of 4-(azidomethyl)-2-(trifluoromethyl)pyridine (190 mg, 0.94 mmol, 1 eq.) in MeOH (10 mL) was added SnCl₂·2H₂O (492.2 mg, 2.16 mmol, 2.3 eq.) at 0° C. The resulting mixture was stirred at room temperature for 3 h under nitrogen atmosphere. After completion of reaction monitored by LCMS. The residue was purified by Prep-HPLC. This resulted in 1-[2-(trifluoromethyl)pyridin-4-yl]methanamine (80 mg, 48.3%) as a white solid. MS: m/z: Calc'd for C₇H₇F₃N₂[M+H]⁺177. found 177.

### Step 4 and 5

The title compound was prepared in 29.9% overall yield as light yellow semi-solid according to Substitution reaction for synthesis of Carbamate; General Procedure I using 1-[2-(trifluoromethyl)pyridin-4-yl]methanamine (100-4) in STEP 4; Boc Deprotection; General Procedure II in STEP 5. MS: m/z Calc'd for C₂₀H₂₂F₃N₃O₄[M+H]⁺426. found 426. ¹H NMR (400 MHz, Methanol-d₄) δ 8.69 (d, J=5.0 Hz, 1H), 7.80 (s, 1H), 7.62 (d, J=5.1 Hz, 1H), 7.22 (d, J=8.5 Hz, 2H), 6.93-6.85 (m, 2H), 4.97 (d, J=3.5 Hz, 1H), 4.57-4.46 (m, 2H), 4.46-4.39 (m, 1H), 4.23-4.14 (m, 1H), 3.79 (s, 3H), 3.62 (dd, J=12.7, 4.3 Hz, 1H), 3.25 (d, J=12.7 Hz, 1H), 3.13 (dd, J=14.1, 7.5 Hz, 1H), 3.01 (dd, J=14.1, 8.2 Hz, 1H).

Prep-HPLC purification conditions: Column: Welch Ultimate XB-C18 50*250, 10 μm; Mobile Phase A: Water (0.05% TFA), Mobile Phase B: ACN; Flow rate: 60 mL/min; Gradient: 2% B to 20% B in 20 min; Wave Length: 254 nm/220 nm; RT1(min): 6.93.

### Synthetic Procedure of (2R,3S,4S)-2-[(3-fluoro-4-methoxyphenyl)methyl]-4-hydroxypyrrolidin-3-yl N-[(3S)-pyrrolidin-3-ylmethyl]carbamate (V188076)

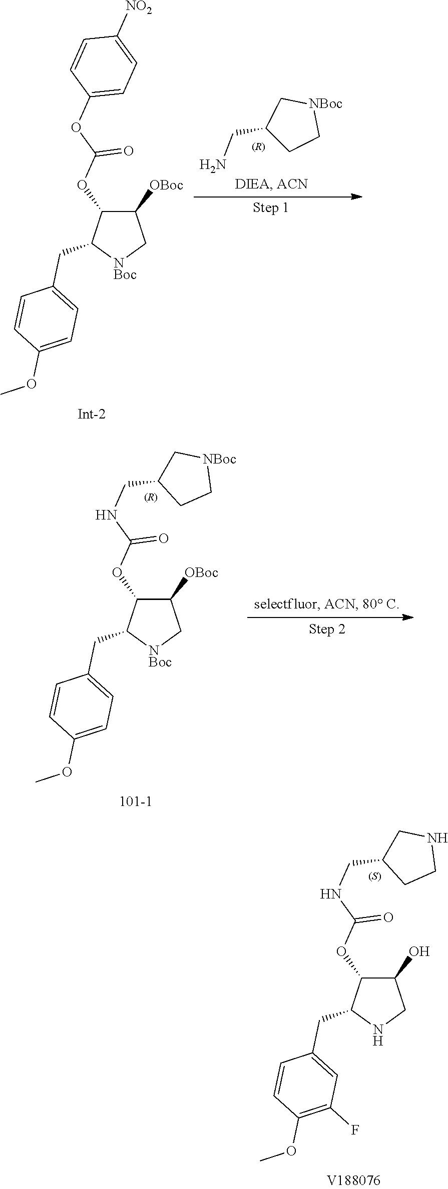

Step 1: The compound was prepared in 89% yield as light yellow solid according to Substitution reaction for synthesis of Carbamate; General Procedure I using tert-butyl (3R)-3-(aminomethyl)pyrrolidine-1-carboxylate. MS: m/z: Calc'd for C₃₃H₅₁N₃O₁₀ [M+H+22]⁺672. found 672.

Step 2: A solution of tert-butyl (2R,3S,4S)-4-[(tert-butoxycarbonyl)oxy]-3-[({[(3R)-1-(tert-butoxycarbonyl)pyrrolidin-3-yl]methyl}carbamoyl)oxy]-2-[(4-methoxyphenyl)methyl]pyrrolidine-1-carboxylate (215 mg, 0.33 mmol, 1 eq.) and Selectfluor (234.4 mg, 0.66 mmol, 2 eq.) in ACN (10 mL) was stirred for 12 h at 80° C. under nitrogen atmosphere. After completion of reaction monitored by LCMS. The residue was purified by Prep-HPLC. This resulted in (2R,3S,4S)-2-[(3-fluoro-4-methoxyphenyl)methyl]-4-hydroxypyrrolidin-3-yl N-[(3S)-pyrrolidin-3-ylmethyl]carbamate (8.9 mg, 7.3%) as a brown solid. MS: m/z: Calc'd for C₁₈H₂₆FN₃O₄[M+H]⁺ 368. found 368. ¹H NMR (400 MHz, Methanol-d₄) δ 7.14-7.05 (m, 3H), 4.96 (d, J=3.5 Hz, 1H), 4.39 (d, J=4.2 Hz, 1H), 4.23-4.14 (m, 1H), 3.88 (s, 3H), 3.59 (dd, J=12.7, 4.2 Hz, 1H), 3.49-3.38 (m, 2H), 3.30-3.20 (m, 4H), 3.11 (dd, J=14.2, 7.2 Hz, 1H), 3.05-2.96 (m, 2H), 2.68-2.56 (m, 1H), 2.26-2.13 (m, 1H), 1.86-1.72 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 5% B to 25% B in 10 min; Wave Length: 254 nm/220 nm; RT1(min): 8.38.

### Synthetic Procedure of (2R,3S,4S)-4-hydroxy-2-[(4-methoxyphenyl)methyl]pyrrolidin-3-yl N-{[(3S)-4,4-difluoropyrrolidin-3-yl]methyl}carbamate (V188094)

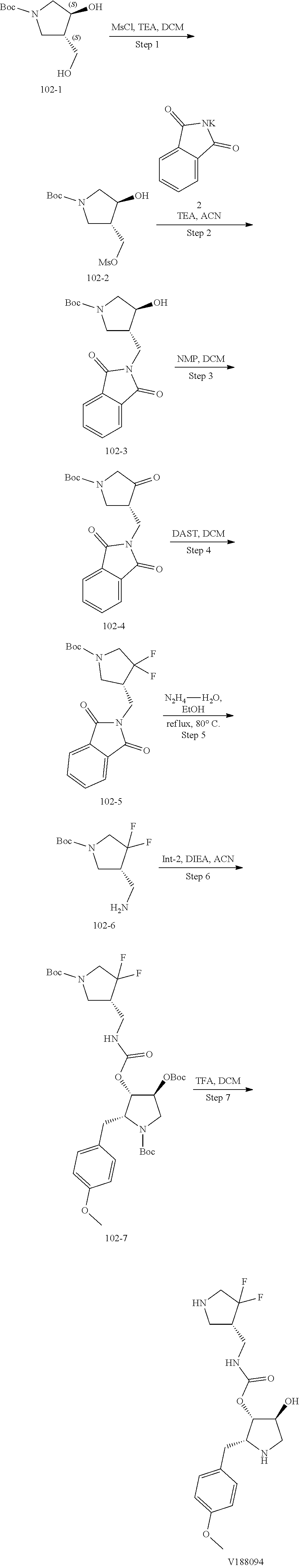

Step 1: To a stirred solution of tert-butyl (3S,4S)-3-hydroxy-4-(hydroxymethyl)pyrrolidine-1-carboxylate (102-1, 2 g, 9.21 mmol, 1 eq.) and TEA (1.4 g, 13.81 mmol, 1.5 eq.) in DCM (30 mL) was added MsCl (1.27 g, 11.05 mmol, 1.2 eq.) add at 0° C. The resulting mixture was stirred at 0° C. under nitrogen atmosphere for 1 h. After completion of reaction monitored by LCMS. The aqueous layer was extracted with EtOAc (3×10 mL). The crude product was used in the next step directly without further purification. MS: m/z: Calc'd for C₁₁H₂₁NO₆S [M+H−56]⁺240. found 240.

Step 2: To a stirred solution of tert-butyl (3S,4S)-3-hydroxy-4-[(methanesulfonyloxy)methyl]pyrrolidine-1-carboxylate (102-2, 2.8 g, 9.48 mmol, 1 eq.) and 2-potassioisoindole-1,3-dione (2.63 g, 14.22 mmol, 1.5 eq.) in ACN (100 mL) was added NaHCO₃ (2.39 g, 28.44 mmol, 3 eq.) add at room temperature. The resulting mixture was stirred at 80° C. for 12 h under nitrogen atmosphere. After completion of reaction monitored by LCMS. The residue was purified by flash. This resulted in tert-butyl (3R,4S)-3-[(1,3-dioxoisoindol-2-yl)methyl]-4-hydroxypyrrolidine-1-carboxylate (1.1 g, 33.5%) as a light yellow oil. MS: m/z: Calc'd for C₁₈H₂₂N₂O₅ [M+H−100]⁺247. found 247.

Step 3: A solution of tert-butyl (3R,4S)-3-[(1,3-dioxoisoindol-2-yl)methyl]-4-hydroxypyrrolidine-1-carboxylate (102-3, 1.1 g, 3.18 mmol, 1 eq.) and NMP (0.4 g, 3.81 mmol, 1.2 eq.) in DCM (50 mL) was stirred for 1 h at room temperature under nitrogen atmosphere. After completion of reaction monitored by LCMS. The residue was purified by flash. This resulted in tert-butyl (3R)-3-[(1,3-dioxoisoindol-2-yl)methyl]-4-oxopyrrolidine-1-carboxylate (610 mg, 55.8%) as a light yellow oil. MS: m/z: Calc'd for C₁₈H₂₀N₂O₅ [M+H−56]⁺289. found 289.

Step 4: To a stirred solution of tert-butyl (3R)-3-[(1,3-dioxoisoindol-2-yl)methyl]-4-oxopyrrolidine-1-carboxylate (102-4, 400 mg, 1.16 mmol, 1 equiv) in DCM (5 mL) was added DAST (1 mL) in portions at 0° C. under nitrogen atmosphere. The resulting mixture was stirred at room temperature for 1 h. After completion of reaction monitored by LCMS. The reaction was quenched with Water at 0° C. The residue was purified by flash. This resulted in tert-butyl (4R)-4-[(1,3-dioxoisoindol-2-yl)methyl]-3,3-difluoropyrrolidine-1-carboxylate (60 mg, 14.1%) as a yellow oil. MS: m/z: Calc'd for C₁₈H₂₀F₂N₂O₄[M+H−56]⁺311. found 311.

Step 5: A solution of tert-butyl (4R)-4-[(1,3-dioxoisoindol-2-yl)methyl]-3,3-difluoropyrrolidine-1-carboxylate (102-5, 60 mg, 0.16 mmol, 1 eq.) and hydrazine hydrate (40.99 mg, 0.82 mmol, 5 eq.) in EtOH (2 mL) was stirred for 3 h at 80° C. under nitrogen atmosphere. After completion of reaction monitored by LCMS. The resulting mixture was filtered, the filter cake was washed with EtOH (2 mL) (3×10 mL). The filtrate was concentrated under reduced pressure. The crude product was used in the next step directly without further purification. MS: m/z: Calc'd for C₁₀H₁₈F₂N₂O₂ [M+H−56]⁺ 181. found [M+H−56]⁺181.

### Step 6 and 7

The title compound was prepared in 46.5% overall yield as a white solid according to Substitution reaction for synthesis of Carbamate; General Procedure I using tert-butyl (4R)-4-(aminomethyl)-3,3-difluoropyrrolidine-1-carboxylate (102-6) in STEP 6; Boc Deprotection; General Procedure II in STEP 7. MS: m/z Calc'd for C₁₈H₂₅F₂N₃O₄ [M+H]⁺494. found 494. ¹H NMR (400 MHz, Methanol-d₄) δ 7.24 (d, J=8.5 Hz, 2H), 6.97-6.89 (m, 2H), 4.97 (d, J=3.9 Hz, 1H), 4.39 (d, J=4.2 Hz, 1H), 4.17 (s, 1H), 3.80 (s, 6H), 3.61-3.50 (m, 2H), 3.47-3.35 (m, 2H), 3.22 (d, J=12.6 Hz, 1H), 3.15-2.96 (m, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 23% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-{[(3R)-1-methylpyrrolidin-3-yl]methyl}carbamate (V188108)

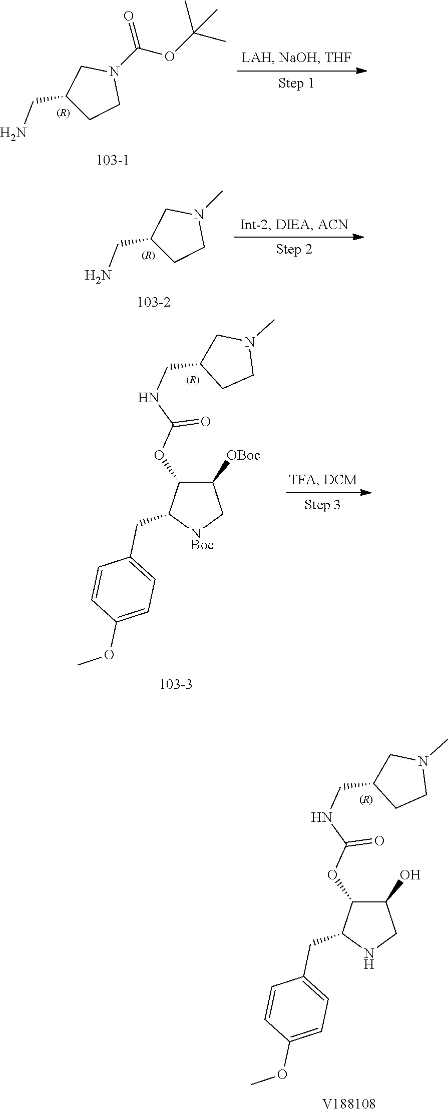

Step 1: To a stirred solution of tert-butyl (3R)-3-(aminomethyl)pyrrolidine-1-carboxylate (200 mg, 0.99 mmol, 1 eq.) in THF (4 mL) was added LAH (4 mL) dropwise at 0° C. under nitrogen atmosphere. The resulting mixture was stirred at 0° C. for 1 h under nitrogen atmosphere. After completion of reaction monitored by LCMS. The resulting mixture was filtered, the filter cake was washed with THF (4 mL). The filtrate was concentrated under reduced pressure. The crude product was used in the next step directly without further purification. MS: m/z: Calc'd for C₆H₁₄N₂ [M+H]⁺115. found 115.

Step 2 and 3: The title compound was prepared in 8.2% overall yield as a light yellow solid according to Substitution reaction for synthesis of Carbamate; General Procedure I using 1-[(3R)-1-methylpyrrolidin-3-yl]methanamine (103-2) in STEP 2; Boc Deprotection; General Procedure II in STEP 3. MS: m/z Calc'd for C₁₉H₂₉N₃O₄ [M+H]⁺364. found 364. ¹H NMR (400 MHz, Methanol-d₄) δ 7.30-7.18 (m, 2H), 6.99-6.88 (m, 2H), 4.97 (d, J=3.5 Hz, 1H), 4.39 (d, J=4.3 Hz, 1H), 4.22-4.12 (m, 1H), 3.79 (s, 3H), 3.58 (dd, J=12.7, 4.3 Hz, 1H), 3.32-3.25 (m, 5H), 3.23-3.19 (m, 1H), 3.11 (dd, J=14.2, 7.1 Hz, 1H), 3.03-2.92 (m, 4H), 2.82-2.62 (m, 1H), 2.28 (s, 1H), 1.89 (s, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 17% B in 10 min; Wave Length: 254 nm/220 nm; RT1(min): 9.53.

### Synthetic Procedure of: (2R,3S,4S)-4-hydroxy-2-[(4-methoxyphenyl)methyl]pyrrolidin-3-yl N-(JH-1,3-benzodiazol-5-ylmethyl)carbamate (V188150)

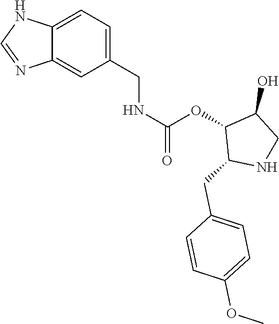 [SMILES: COc1ccc(C[C@H]2NC[C@H](O)[C@H]2OC(=O)NCc2ccc3[nH]cnc3c2)cc1]

The title compound was prepared in 11.8% overall yield as a light yellow solid according to Substitution reaction for synthesis of Carbamate; General Procedure I using 1-(1H−1,3-benzodiazol-5-yl)methanamine in STEP 1; Boc Deprotection; General Procedure II in STEP 2. MS: m/z Calc'd for C₂₁H₂₄N₄O₄ [M+H]⁺ 397. found 397. ¹H NMR (400 MHz, Methanol-d₄) δ 9.23 (s, 1H), 7.83 (d, J=10.6 Hz, 2H), 7.62 (d, J=8.3 Hz, 1H), 7.19 (d, J=8.4 Hz, 2H), 6.87-6.79 (m, 2H), 4.96 (s, 1H), 4.59 (s, 1H), 4.51 (s, 1H), 4.40 (d, J=4.2 Hz, 1H), 4.17 (d, J=3.6 Hz, 1H), 3.77 (s, 3H), 3.63-3.55 (m, 1H), 3.23 (d, J=12.7 Hz, 1H), 3.10 (dd, J=14.1, 7.4 Hz, 1H), 2.97 (dd, J=13.9, 8.4 Hz, 1H), 2.12 (s, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 9.85.

### Synthetic Procedure of (2R,3S,4S)-2-[(4-chlorophenyl)methyl]-4-hydroxypyrrolidin-3-yl N-[(1-fluorocyclopropyl)methyl]carbamate (V188269)

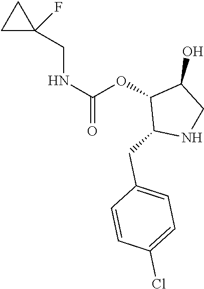 [SMILES: O=C(NCC1(F)CC1)O[C@@H]1[C@@H](O)CN[C@@H]1Cc1ccc(Cl)cc1]

The title compound was prepared in 38.6% overall yield as a white solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(1-fluorocyclopropyl)methanamine in STEP 1; Boc Deprotection; General Procedure II in STEP 2. MS: m/z Calc'd for C₁₆H₂₀ClFN₂O₃[M+H]⁺343. found 343. ¹H NMR (400 MHz, Methanol-d₄) δ 7.42-7.30 (m, 4H), 4.40 (d, J=4.2 Hz, 1H), 4.27-4.17 (m, 1H), 3.64-3.46 (m, 3H), 3.29-3.14 (m, 2H), 3.06 (dd, J=14.4, 7.9 Hz, 1H), 1.05 (dd, J=18.6, 6.5 Hz, 2H), 0.83-0.73 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 12% B to 42% B in 10 min; Wave Length: 254 nm/220 nm; RT1(min): 9.15.

### Synthetic Procedure of (2R,3S,4S)-2-[(4-chlorophenyl)methyl]-4-hydroxypyrrolidin-3-yl N-(cyclopropylmethyl)carbamate (V188275)

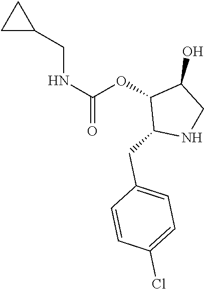 [SMILES: O=C(NCC1CC1)O[C@@H]1[C@@H](O)CN[C@@H]1Cc1ccc(Cl)cc1]

The title compound was prepared in 78.1% overall yield as a white solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-cyclopropylmethanamine in STEP 1; Boc Deprotection; General Procedure II in STEP 2. MS: m/z Calc'd for C₁₆H₂₁ClN₂O₃[M+H]⁺325. found 325. ¹H NMR (400 MHz, Methanol-d₄) δ 7.42-7.30 (m, 4H), 4.40 (d, J=4.3 Hz, 1H), 4.25-4.17 (m, 1H), 3.63-3.54 (m, 1H), 3.28-3.14 (m, 2H), 3.11-2.97 (m, 3H), 1.10-0.95 (m, 1H), 0.59-0.50 (m, 2H), 0.29-0.21 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 12% B to 42% B in 10 min; Wave Length: 254 nm/220 nm; RT1(min): 9.15.

### Synthetic Procedure of (2R,3S,4S)-2-[(4-chlorophenyl)methyl]-4-hydroxypyrrolidin-3-yl N-{[4-(trifluoromethoxy)phenyl]methyl}carbamate (V188286)

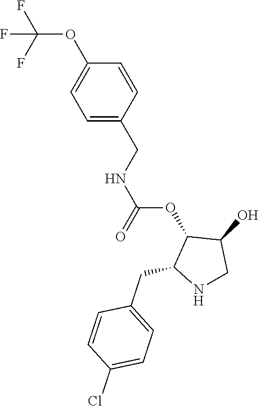 [SMILES: O=C(NCc1ccc(OC(F)(F)F)cc1)O[C@@H]1[C@@H](O)CN[C@@H]1Cc1ccc(Cl)cc1]

The title compound was prepared in 69.5% overall yield as a light yellow solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-[4-(trifluoromethoxy)phenyl]methanamine in STEP 1; Boc Deprotection; General Procedure II in STEP 2. MS: m/z Calc'd for C₂₀H₂₀ClF₃N₂O₄ [M+H]⁺445. found 445. ¹H NMR (400 MHz, Methanol-d₄) δ 7.48-7.41 (m, 2H), 7.35-7.25 (m, 6H), 4.96 (d, J=3.4 Hz, 1H), 4.44-4.17 (m, 4H), 3.60 (dd, J=12.6, 4.2 Hz, 1H), 3.24 (d, J=12.7 Hz, 1H), 3.16 (dd, J=14.1, 7.5 Hz, 1H), 3.10-3.00 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 24% B to 54% B in 10 min; Wave Length: 254 nm/220 nm; RT1(min): 9.02.

### Synthetic Procedure of (2R,3S,4S)-2-[(4-chlorophenyl)methyl]-4-hydroxypyrrolidin-3-yl N-[(3S)-oxolan-3-ylmethyl]carbamate (V188288)

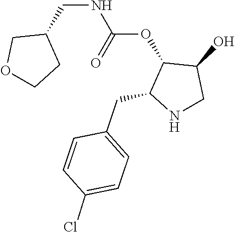 [SMILES: O=C(NC[C@@H]1CCOC1)O[C@@H]1[C@@H](O)CN[C@@H]1Cc1ccc(Cl)cc1]

The title compound was prepared in 69.5% overall yield as a light yellow solid according to Substitution reaction for synthesis of Carbamate; General Procedure I using tert-butyl (2R,3S,4S)-4-[(tert-butoxycarbonyl)oxy]-2-[(4-chlorophenyl)methyl]-3-[(4-nitrophenoxycarbonyl)oxy]pyrrolidine-1-carboxylate (Int-11) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-[(3S)-oxolan-3-yl]methanamine in STEP 1; Boc Deprotection; General Procedure II in STEP 2. MS: m/z Calc'd for C₁₇H₂₃ClN₂O₄[M+H]⁺355. found 355. ¹H NMR (400 MHz, Methanol-d₄) δ 7.43-7.36 (m, 2H), 7.32 (d, J=8.5 Hz, 2H), 4.95 (d, J=3.6 Hz, 1H), 4.39 (d, J=4.3 Hz, 1H), 4.26-4.17 (m, 1H), 3.93-3.71 (m, 3H), 3.63-3.52 (m, 2H), 3.28-3.13 (m, 4H), 3.05 (dd, J=14.1, 8.4 Hz, 1H), 2.56-2.44 (m, 1H), 2.13-2.01 (m, 1H), 1.74-1.61 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 6% B to 36% B in 10 min; Wave Length: 254 nm/220 nm; RT1(min): 9.35.

### Synthetic Procedure of (2R,3S,4S)-4-hydroxy-2-{[4-(1H−1,2,3-triazol-4-yl)phenyl]methyl}pyrrolidin-3-yl N-benzylcarbamate (V188331)

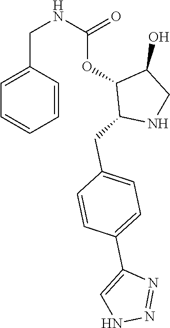 [SMILES: O=C(NCc1ccccc1)O[C@@H]1[C@@H](O)CN[C@@H]1Cc1ccc(-c2c[nH]nn2)cc1]

The title compound was prepared in 36.9% overall yield as a white solid according to Substitution reaction for synthesis of Carbamate; General Procedure I using tert-butyl (2R,3S,4S)-4-[(tert-butoxycarbonyl)oxy]-2-[(4-ethynylphenyl)methyl]-3-[(4-nitrophenoxycarbonyl)oxy]pyrrolidine-1-carboxylate (2-7) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), benzylamine in STEP 1; Cyclization reaction for synthesis of Substituted triazoles; General Procedure III using azidotrimethylsilane in STEP 2; Boc Deprotection; General Procedure II in STEP 3. MS: m/z C₂₁H₂₃N₅O₃ [M+H]⁺394. found 394. ¹H NMR (400 MHz, Methanol-d₄) δ 8.16 (s, 1H), 7.83 (d, J=8.1 Hz, 2H), 7.43-7.32 (m, 6H), 7.31-7.25 (m, 1H), 5.02-4.96 (m, 1H), 4.45-4.21 (m, 4H), 3.61 (dd, J=12.7, 4.3 Hz, 1H), 3.29-3.17 (m, 2H), 3.10 (dd, J=14.0, 8.3 Hz, 1H).

Prep-HPLC purification conditions: Xselect CSH Prep C18 Column, 30*250 mm, 10 μm; Mobile Phase A: Water (0.05% TFA), Mobile Phase B: ACN; Flow rate: 60 mL/min; Gradient: 6% B to 36% B in 10 min; Wave Length: 254 nm/220 nm; RT1(min): 9.5.

### Synthetic Procedure of (2R,3S,4S)-4-hydroxy-2-{[4-(1H−1,2,3-triazol-4-yl)phenyl]methyl}pyrrolidin-3-yl N-(cyclopropylmethyl)carbamate (V188327)

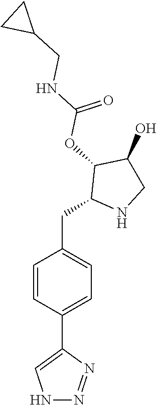 [SMILES: O=C(NCC1CC1)O[C@@H]1[C@@H](O)CN[C@@H]1Cc1ccc(-c2c[nH]nn2)cc1]

The title compound was prepared in 36.9% overall yield as a white solid according to Substitution reaction for synthesis of Carbamate; General Procedure I using tert-butyl (2R,3S,4S)-4-[(tert-butoxycarbonyl)oxy]-2-[(4-ethynylphenyl)methyl]-3-[(4-nitrophenoxycarbonyl)oxy]pyrrolidine-1-carboxylate (2-7) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-cyclopropylmethanamine in STEP 1; Cyclization reaction for synthesis of Substituted triazoles; General Procedure III using Azidotrimethylsilane in STEP 2; Boc Deprotection; General Procedure II in STEP 3. MS: m/z C₁₈H₂₃N₅O₃ [M+H]⁺358. found 358. ¹H NMR (400 MHz, Methanol-d₄) δ 8.18 (s, 1H), 7.87 (d, J=7.9 Hz, 2H), 7.44 (d, J=8.0 Hz, 2H), 4.99 (d, J=3.5 Hz, 1H), 4.41 (d, J=4.2 Hz, 1H), 4.30-4.20 (m, 1H), 3.61 (dd, J=12.7, 4.3 Hz, 1H), 3.29-3.19 (m, 2H), 3.15-2.98 (m, 3H), 1.10-0.98 (m, 1H), 0.60-0.51 (m, 2H), 0.30-0.23 (m, 2H).

Prep-HPLC purification conditions: Xselect CSH Prep C18 Column, 30*250 mm, 10 μm; Mobile Phase A: Water (0.05% TFA), Mobile Phase B: ACN; Flow rate: 60 mL/min; Gradient: 2% B to 31% B in 10 min; Wave Length: 254 nm/220 nm; RT1(min): 9.3.

### Synthetic Procedure of (2R,3S,4S)-4-hydroxy-2-[(4-methoxyphenyl)methyl]pyrrolidin-3-yl N-{[(3R)-1-methylpyrrolidin-3-yl]methyl}carbamate (V188306)

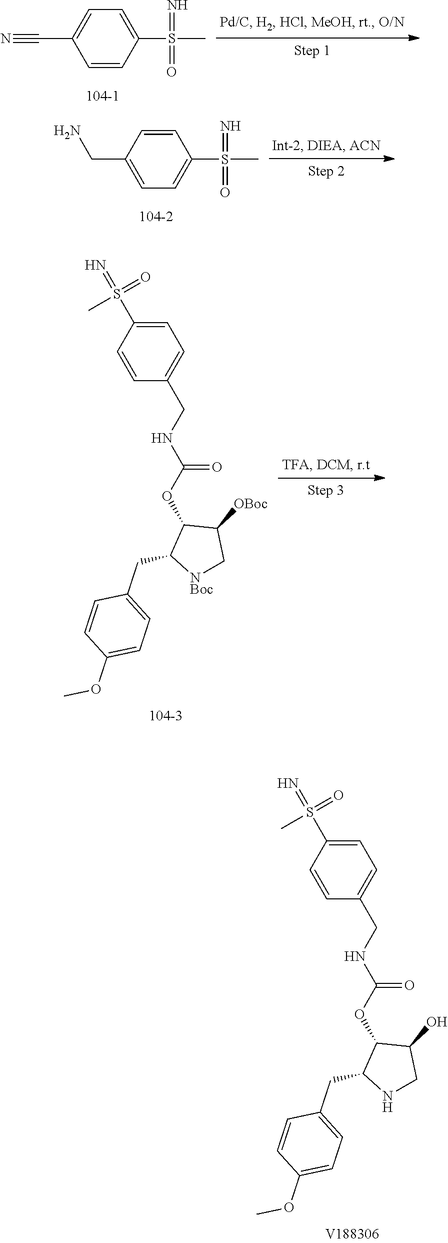

Step 1: To a stirred mixture of 4-[imino(methyl)oxo-lambda6-sulfanyl]benzonitrile (120 mg, 0.66 mmol, 1 equiv) and HCl (6M) (2.2 mg, 0.01 mmol, 0.02 equiv) in MeOH (5 mL) was added Pd/C (35.4 mg, 0.33 mmol, 0.5 equiv) at room temperature under nitrogen atmosphere. The resulting mixture was stirred for overnight at room temperature under hydrogen atmosphere. The resulting mixture was filtered, the filter cake was washed with MeOH (5 mL) (1×2 mL). The filtrate was concentrated under reduced pressure. to afford [4-(aminomethyl)phenyl](imino)methyl-lambda6-sulfanone (110 mg, 89.6%) as a yellow oil.

Step 2 and 3: The title compound was prepared in 53.3% overall yield as a light yellow solid according to Substitution reaction for synthesis of Carbamate; General Procedure I using [4-(aminomethyl)phenyl](imino)methyl-lambda6-sulfanone (104-2) in STEP 2; Boc Deprotection; General Procedure II in STEP 3. MS: m/z Calc'd for C₂₁H₂₇N₃O₅S [M+H]⁺434. found 434. ¹H NMR (400 MHz, Methanol-d₄) δ 8.13-8.05 (m, 2H), 7.72-7.64 (m, 2H), 7.24-7.17 (m, 2H), 6.92-6.84 (m, 2H), 4.94 (d, J=3.5 Hz, 1H), 4.55-4.41 (m, 2H), 4.38 (d, J=4.1 Hz, 1H), 4.21-4.12 (m, 1H), 3.78 (s, 3H), 3.66-3.45 (m, 4H), 3.22 (d, J=12.6 Hz, 1H), 3.10 (dd, J=14.1, 7.5 Hz, 1H), 3.04-2.93 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 28% B in 10 min; Wave Length: 254 nm/220 nm; RT1(min): 8.9.

### Synthetic Procedure of (2R,3S,4S)-4-hydroxy-2-[(4-methoxyphenyl)methyl]pyrrolidin-3-ylN-{[(3R)-1-methylpyrrolidin-3-yl]methyl}carbamate (V188339)

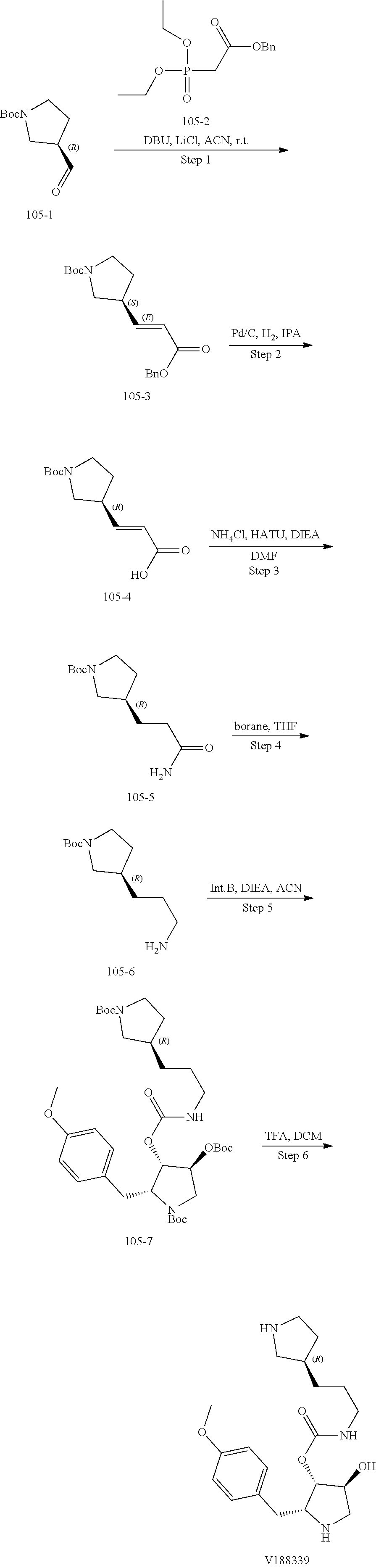

Step 1: A solution of benzyl 2-(diethoxyphosphoryl)acetate (105-2, 1436.71 mg, 5.018 mmol, 2 equiv) in ACN (10 mL) was treated with DBU (458.4 mg, 3.01 mmol, 1.2 eq.) and LiCl (148.9 mg, 3.51 mmol, 1.4 eq.) for 30 min at 0° C. under nitrogen atmosphere followed by the addition of tert-butyl (3R)-3-formylpyrrolidine-1-carboxylate (105-1, 500 mg, 2.509 mmol, 1 equiv) in portions at 0° C. The resulting mixture was stirred at room temperature for 6 h under nitrogen atmosphere. Desired product could be detected by TLC. The residue was purified by Prep-TLC (PE:EA, 3:1). This resulted in tert-butyl (3S)-3-[3-(benzyloxy)-3-oxoprop-1-en-1-yl]pyrrolidine-1-carboxylate (560 mg, 67.3%) as a yellow oil.

Step 2: To a stirred solution of tert-butyl (3S)-3-[3-(benzyloxy)-3-oxoprop-1-en-1-yl]pyrrolidine-1-carboxylate (105-3, 550 mg, 1.66 mmol, 1 eq.) in IPA (15 mL) was added Pd/C (110 mg, 1.03 mmol, 0.6 eq.) at room temperature under nitrogen atmosphere. The resulting mixture was stirred at room temperature for 2 h under hydrogen atmosphere. After completion of reaction monitored by LCMS. The precipitated solids were collected by filtration and washed with IPA (15 mL) (3×10 mL). This resulted in 3-[(3R)-1-(tert-butoxycarbonyl)pyrrolidin-3-yl]propanoic acid (550 mg, 136.2%) as a colorless oil. MS: m/z: Calc'd for C₁₂H₂₁NO₄ [M+H−56]⁺188. found 188.

Step 3: To a stirred solution of 3-[(3R)-1-(tert-butoxycarbonyl)pyrrolidin-3-yl]propanoic acid (105-4, 395 mg, 1.62 mmol, 1 eq.) and NH4Cl (86.8 mg, 1.62 mmol, 1 eq.) in DMF (20 mL) were added HATU (1234.6 mg, 3.25 mmol, 2 eq.) and DIEA (839.3 mg, 6.49 mmol, 4 eq.) at room temperature. The resulting mixture was stirred at room temperature for 4 h under nitrogen atmosphere. After completion of reaction monitored by LCMS. The residue was purified by flash. This resulted in tert-butyl (3R)-3-(2-carbamoylethyl)pyrrolidine-1-carboxylate (60 mg, 15.3%) as a yellow oil. MS: m/z: Calc'd for C₁₂H₂₂N₂O₃ [M+H−56]⁺187. found 187.

Step 4: To a stirred solution of tert-butyl (3R)-3-(2-carbamoylethyl)pyrrolidine-1-carboxylate (105-5, 295 mg, 1.22 mmol, 1 eq.) in THF (10 mL) was added borane-THF (313.9 mg, 3.65 mmol, 3 eq.) at 0° C. under nitrogen atmosphere. The resulting mixture was stirred at room temperature for 4 h under nitrogen atmosphere. After completion of reaction monitored by LCMS. The reaction was quenched by the addition of MeOH (2 mL) at 0° C. The resulting mixture was concentrated under reduced pressure. The crude product was used in the next step directly without further purification. MS: m/z: Calc'd for C₁₂H₂₄N₂O₂ [M+H]⁺229. found 229.

### Step 5 and 6

The title compound was prepared in 26.4% overall yield as a light yellow solid according to Substitution reaction for synthesis of Carbamate; General Procedure I using tert-butyl (3R)-3-(3-aminopropyl)pyrrolidine-1-carboxylate (105-6) in STEP 5; Boc Deprotection; General Procedure II in STEP 6. MS: m/z Calc'd for C₂₀H₃₁N₃O₄ [M+H]⁺378. found 378. ¹H NMR (400 MHz, Methanol-d₄) δ 7.27-7.20 (m, 2H), 6.97-6.89 (m, 2H), 4.95 (d, J=3.5 Hz, 1H), 4.38 (d, J=4.2 Hz, 1H), 4.22-4.11 (m, 1H), 3.80 (s, 3H), 3.57 (dd, J=12.7, 4.3 Hz, 1H), 3.49-3.37 (m, 2H), 3.29-3.08 (m, 5H), 2.97 (dd, J=14.1, 8.5 Hz, 1H), 2.83 (dd, J=11.5, 9.3 Hz, 1H), 2.43-2.30 (m, 1H), 2.29-2.16 (m, 1H), 1.71-1.43 (m, 5H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 19% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-{3-[(3S)-pyrrolidin-3-yl]propyl}carbamate (V188340)

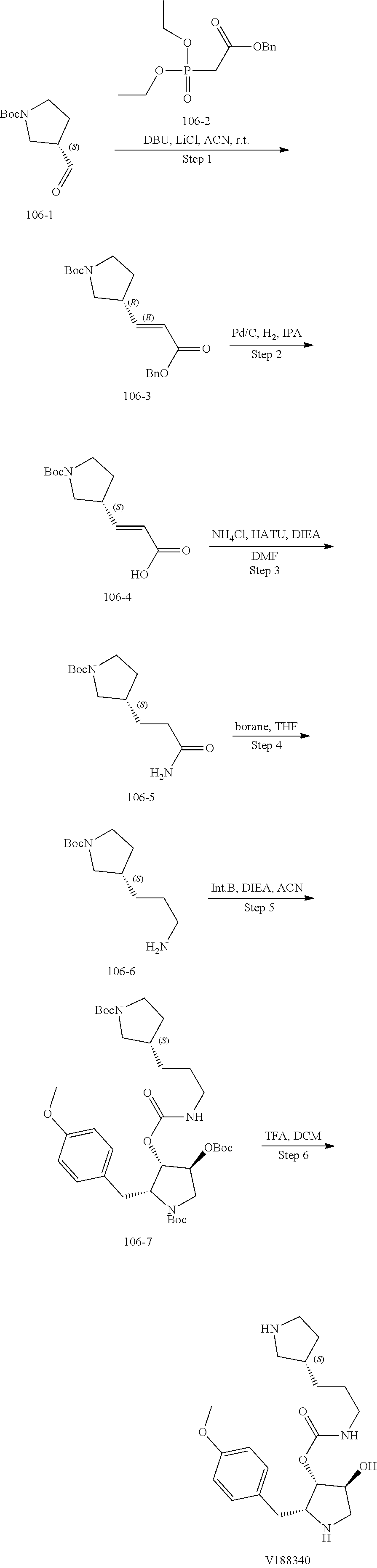

Step 1: A solution of benzyl 2-(diethoxyphosphoryl)acetate (106-2, 1436.7 mg, 5.02 mmol, 2 eq.) in ACN (10 mL) was treated with DBU (458.4 mg, 3.01 mmol, 1.2 eq.) and LiCl (148.9 mg, 3.51 mmol, 1.4 eq.) at 0° C. for 30 min under nitrogen atmosphere followed by the addition of tert-butyl (3S)-3-formylpyrrolidine-1-carboxylate (500 mg, 2.51 mmol, 1 eq.) in portions at 0° C. The resulting mixture was stirred at room temperature for 6 h under nitrogen atmosphere. After completion of reaction monitored by LCMS. The residue was purified by Prep-TLC (PE:EA, 3:1). This resulted in tert-butyl (3R)-3-[3-(benzyloxy)-3-oxoprop-1-en-1-yl]pyrrolidine-1-carboxylate (440 mg, 52.9%) as a colorless oil.

Step 2: To a stirred solution of tert-butyl (3R)-3-[3-(benzyloxy)-3-oxoprop-1-en-1-yl]pyrrolidine-1-carboxylate (106-3, 430 mg, 1.29 mmol, 1 eq.) in IPA (10 mL) was added Pd/C (110 mg, 1.03 mmol, 0.8 eq.) at room temperature under nitrogen atmosphere. The resulting mixture was stirred at room temperature for 2 h under hydrogen atmosphere. After completion of reaction monitored by LCMS. The precipitated solids were collected by filtration and washed with IPA (10 mL) (3×10 mL). This resulted in 3-[(3S)-1-(tert-butoxycarbonyl)pyrrolidin-3-yl]propanoic acid (346 mg, 109.6%) as a colorless oil. MS: m/z: Calc'd for C₁₂H₂₁NO₄ [M+H−56]⁺188. found 188.

Step 3: To a stirred solution of 3-[(3S)-1-(tert-butoxycarbonyl)pyrrolidin-3-yl]propanoic acid (106-4, 315 mg, 1.3 mmol, 1 eq.) and NH4Cl (69.3 mg, 1.3 mmol, 1 eq.) in DMF (20 mL) was added HATU (984.6 mg, 2.59 mmol, 2 eq.) and DIEA (669.3 mg, 5.18 mmol, 4 eq.) dropwise at room temperature. The resulting mixture was stirred at room temperature for 4 h under nitrogen atmosphere. After completion of reaction monitored by LCMS. The aqueous layer was extracted with EtOAc (3×5 mL). The crude product was used in the next step directly without further purification. This resulted in tert-butyl (3S)-3-(2-carbamoylethyl)pyrrolidine-1-carboxylate (260 mg, 82.9%) as a colorless oil. MS: m/z: Calc'd for C₁₂H₂₂N₂O₃ [M+H−56]⁺ 187. found 187.

Step 4: To a stirred solution of tert-butyl (3S)-3-(2-carbamoylethyl)pyrrolidine-1-carboxylate (106-5, 260 mg, 1.07 mmol, 1 eq.) in THF (10 mL) was added borane-THF (276.6 mg, 3.22 mmol, 3 eq.) at 0° C. The resulting mixture was stirred at room temperature for 4 h under nitrogen atmosphere. After completion of reaction monitored by LCMS. The resulting mixture was concentrated under reduced pressure. The crude product was used in the next step directly without further purification. MS: m/z: Calc'd for C₁₂H₂₄N₂O₂ [M+H]⁺229. found 229.

### Step 5 and 6

The title compound was prepared in 33.4% overall yield as an off-white solid according to Substitution reaction for synthesis of Carbamate; General Procedure I using tert-butyl (3S)-3-(3-aminopropyl)pyrrolidine-1-carboxylate (106-6) in STEP 5; Boc Deprotection; General Procedure II in STEP 6. MS: m/z Calc'd for C₂₀H₃₁N₃O₄ [M+H]⁺378. found 378. ¹H NMR (400 MHz, Methanol-d₄) δ 7.28-7.19 (m, 2H), 6.96-6.89 (m, 2H), 4.97-4.91 (m, 1H), 4.37 (d, J=4.3 Hz, 1H), 4.19-4.11 (m, 1H), 3.80 (s, 3H), 3.59 (dd, J=12.6, 4.4 Hz, 1H), 3.51-3.34 (m, 2H), 3.30-3.08 (m, 5H), 2.99 (dd, J=14.1, 8.4 Hz, 1H), 2.83 (dd, J=11.5, 9.3 Hz, 1H), 2.43-2.27 (m, 1H), 2.27-2.16 (m, 1H), 1.70-1.43 (m, 5H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 15% B in 10 min; Wave Length: 254 nm/220 nm; RT1(min): 11.5.

### Synthetic Procedure of (2R,3S,4S)-4-hydroxy-2-[(4-methoxyphenyl)methyl]pyrrolidin-3-yl N-{[4-(pyrrolidin-3-yl)phenyl]methyl}carbamate (V188354)

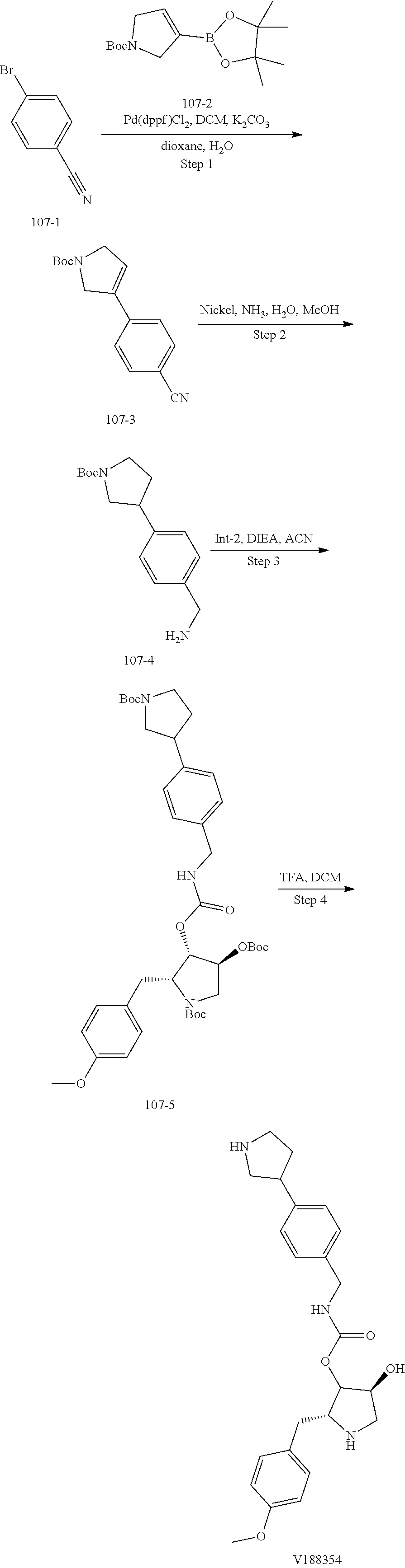

Step 1: To a stirred solution of 4-bromobenzonitrile (107-1, 500 mg, 2.75 mmol, 1.0 eq.) and tert-butyl 3-(4,4,5,5-tetramethyl-1,3,2-dioxaborolan-2-yl)-2,5-dihydropyrrole-1-carboxylate (107-2, 1216.3 mg, 4.12 mmol, 1.5 eq.) in dioxane (10 mL) and H₂O (1 mL) was added Pd(dppf)Cl₂ (201.00 mg, 0.28 mmol, 0.1 eq.) and K₂CO₃ (759.3 mg, 5.49 mmol, 2.0 eq.) in portions at room temperature. The resulting mixture was stirred over night at 80° C. under a nitrogen atmosphere. The resulting mixture was filtered, the filter cake was washed with dioxane (10 mL) (3×3 mL). The filtrate was concentrated under reduced pressure. The residue was purified by reversed-phase flash chromatography to afford tert-butyl 3-(4-cyanophenyl)-2,5-dihydropyrrole-1-carboxylate (700 mg, 94.3%) as a yellow solid. MS: m/z: Calc'd for C₁₆H₁₈N₂O₂[M+H−56+41]⁺256. Found, 256.

Step 2: To a stirred solution of tert-butyl 3-(4-cyanophenyl)-2,5-dihydropyrrole-1-carboxylate (107-3, 100 mg, 0.370 mmol, 1.0 eq.) in MeOH (2 mL) was added Raney Ni (95.08 mg, 1.110 mmol, 3.0 eq.) and NH₃·H₂O (18.9 mg, 1.11 mmol, 3.0 eq.) in portions at room temperature. The resulting mixture was stirred over night at 25° C. under N₂. The resulting mixture was filtered, the filter cake was washed with MeOH (2×2 mL). The filtrate was concentrated under reduced pressure. This resulted in tert-butyl 3-[4-(aminomethyl)phenyl]pyrrolidine-1-carboxylate (100 mg, 97.8%) as a light yellow oil. MS: m/z: Calc'd for C₁₆H₂₄N₂O₂ [M*2+H]⁺553. Found, 553.

Step 3 and 4: The title compound was prepared in 33.4% overall yield as a white solid according to Substitution reaction for synthesis of Carbamate; General Procedure I using tert-butyl tert-butyl 3-[4-(aminomethyl)phenyl]pyrrolidine-1-carboxylate (107-4) in STEP 3; Boc Deprotection; General Procedure II in STEP 4. MS: m/z Calc'd for C₂₄H₃₁N₃O₄ [M+H]⁺426. Found, 426. ¹H NMR (400 MHz, Methanol-d₄) δ 7.36 (s, 4H), 7.20 (d, J=8.1 Hz, 2H), 6.87 (d, J=8.0 Hz, 2H), 4.94-4.91 (m, 1H), 4.42-4.24 (m, 3H), 4.18-4.10 (m, 1H), 3.79 (s, 3H), 3.72 (dd, J=11.4, 7.9 Hz, 1H), 3.64-3.49 (m, 3H), 3.44-3.36 (m, 1H), 3.24-3.18 (m, 2H), 3.09 (dd, J=14.0, 7.7 Hz, 1H), 2.98 (dd, J=13.9, 8.4 Hz, 1H), 2.50-2.47 (m, 1H), 2.19-2.01 (m, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 10.18.

### Synthetic Procedure of (2R,3S,4S)-4-hydroxy-2-[(4-methoxyphenyl)methyl]pyrrolidin-3-yl N-{1H,2H,3H-pyrrolo[2,3-b]pyridin-4-ylmethyl}carbamate (V188360)

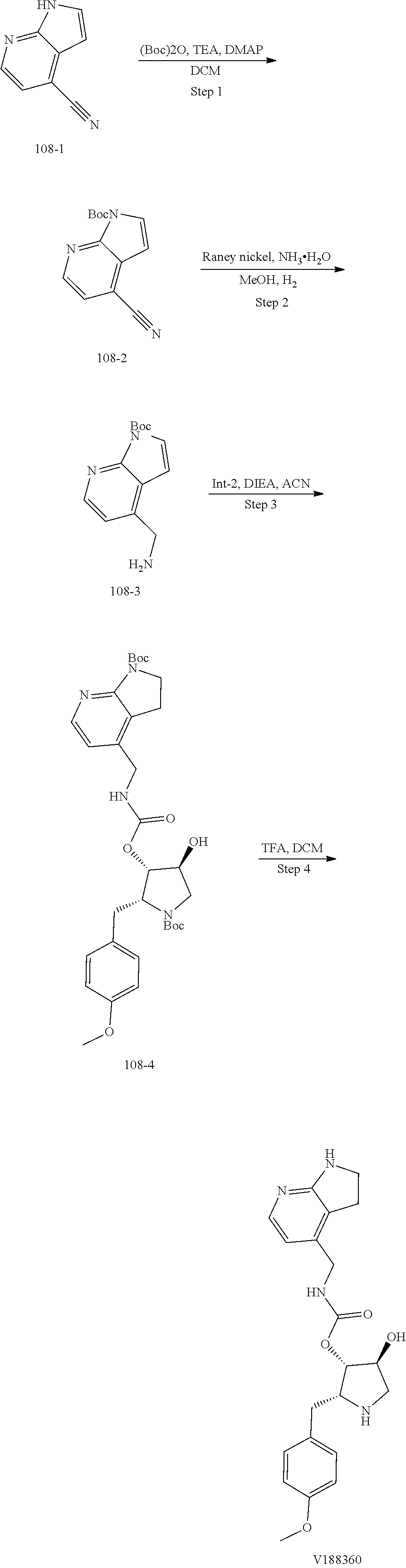 [SMILES: N#Cc1ccnc2[nH]ccc12 ; CC(C)(C)OC(=O)n1ccc2c(C#N)ccnc21 ; CC(C)(C)OC(=O)n1ccc2c(CN)ccnc21 ; COc1ccc(C[C@@H]2[C@H](OC(=O)NCc3ccnc4c3CCN4C(=O)OC(C)(C)C)[C@@H](O)CN2C(=O)OC(C)(C)C)cc1 ; COc1ccc(C[C@H]2NC[C@H](O)[C@H]2OC(=O)NCc2ccnc3c2CCN3)cc1]

Step 1: To a stirred solution of 1H-pyrrolo[2,3-b]pyridine-4-carbonitrile (108-1, 500 mg, 3.493 mmol, 1.0 eq.) in DCM (10 mL) was added TEA (1060.4 mg, 10.48 mmol, 3.0 eq.), DMAP (213.4 mg, 1.75 mmol, 0.5 eq.) and Boc₂O (838.6 mg, 3.84 mmol, 1.1 eq.) in portions at 0° C. The resulting mixture was stirred over night at 25° C. The resulting mixture was concentrated under reduced pressure. The residue was dissolved in ACN (3 mL). The residue was purified by reversed-phase flash chromatography with the following conditions: column, C18 silica gel; mobile phase, MeCN in Water (0.1% TFA), 10% to 50% gradient in 10 min; detector, UV 220 nm. This resulted in tert-butyl 4-cyanopyrrolo[2,3-b]pyridine-1-carboxylate (600 mg, 70.6%) as a white solid. MS: m/z: Calc'd for C₁₃H₁₃N₃O₂ [M+H−56]⁺188. Found, 188.

Step 2: To a stirred solution of tert-butyl 4-cyanopyrrolo[2,3-b]pyridine-1-carboxylate (108-2, 100 mg, 0.411 mmol, 1 eq.) in MeOH (5 mL) was added Raney Ni (100 mg, 1.17 mmol, 2.8 eq.) and NH₃·H₂O (28.8 mg, 0.82 mmol, 2.0 eq.) in portions at 25° C. The resulting mixture was stirred over night at 25° C. under H₂. The resulting mixture was filtered, the filter cake was washed with MeOH (3×3 mL). The filtrate was concentrated under reduced pressure. This resulted in tert-butyl 4-(aminomethyl)-2H,3H-pyrrolo[2,3-b]pyridine-1-carboxylate (50 mg, 48.8%) as a light yellow oil. MS: m/z: Calc'd for C₁₃H₁₉N₃O₂ [M+H]⁺250. Found, 250.

Step 3 and 4: The title compound was prepared in 33.4% overall yield as an off-white solid according to Substitution reaction for synthesis of Carbamate; General Procedure I using tert-butyl 4-(aminomethyl)-2H,3H-pyrrolo[2,3-b]pyridine-1-carboxylate (108-3) in STEP 3; Boc Deprotection; General Procedure II in STEP 4. MS: m/z Calc'd for C₂₁H₂₆N₄O₄ [M+H]⁺399. Found, 399. ¹H NMR (400 MHz, Methanol-d₄) δ 7.54 (d, J=6.8 Hz, 1H), 7.23 (d, J=8.3 Hz, 2H), 6.94-6.86 (m, 2H), 6.68 (d, J=6.8 Hz, 1H), 4.97 (d, J=3.5 Hz, 1H), 4.40 (d, J=4.1 Hz, 1H), 4.35-4.15 (m, 3H), 3.98-3.92 (m, 2H), 3.80 (s, 3H), 3.60 (dd, J=12.5, 4.5 Hz, 1H), 3.31-3.21 (m, 3H), 3.12 (dd, J=14.1, 7.4 Hz, 1H), 3.00 (dd, J=14.2, 8.5 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 23% B in 10 min; Wave Length: 254 nm/220 nm; RT1(min): 9.2.

### Synthetic Procedure of (2R,3S,4S)-4-hydroxy-2-[(4-methoxyphenyl)methyl]pyrrolidin-3-yl N-[(5-chlorothiophen-2-yl)methyl]carbamate (V188373)

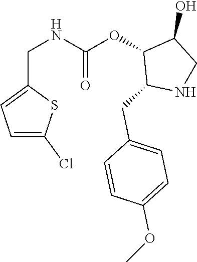 [SMILES: COc1ccc(C[C@H]2NC[C@H](O)[C@H]2OC(=O)NCc2ccc(Cl)s2)cc1]

The title compound was prepared in 36.5% overall yield as a light yellow solid according to Substitution reaction for synthesis of Carbamate; General Procedure I using 1-(5-chlorothiophen-2-yl)methanamine in STEP 1; Boc Deprotection; General Procedure II in STEP 2. MS: m/z Calc'd for C₁₈H₂₁ClN₂O₄S [M+H]⁺397. found 397. ¹H NMR (400 MHz, Methanol-d₄) δ 7.22-7.16 (m, 2H), 6.92-6.83 (m, 4H), 4.93 (s, 1H), 4.50-4.33 (m, 3H), 4.20-4.11 (m, 1H), 3.79 (s, 3H), 3.56 (d, J=12.6 Hz, 1H), 3.22 (d, J=12.7 Hz, 1H), 3.09 (dd, J=13.9, 7.8 Hz, 1H), 3.01-2.91 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 16% B to 46% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-[(1-fluorocyclopropyl)methyl]carbamate (V188381)

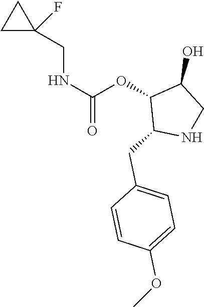 [SMILES: COc1ccc(C[C@H]2NC[C@H](O)[C@H]2OC(=O)NCC2(F)CC2)cc1]

The title compound was prepared in 39.5% overall yield as a white solid according to Substitution reaction for synthesis of Carbamate; General Procedure I using 1-(1-fluorocyclopropyl)methanamine in STEP 1; Boc Deprotection; General Procedure II in STEP 2. MS: m/z Calc'd for C₁₇H₂₃FN₂O₄[M+H]⁺ 339. found 339. ¹H NMR (400 MHz, Methanol-d₄) δ 7.28-7.21 (m, 2H), 6.96-6.88 (m, 2H), 4.39 (d, J=4.2 Hz, 1H), 4.21-4.11 (m, 1H), 3.82-3.77 (m, 3H), 3.65-3.46 (m, 3H), 3.23 (d, J=12.6 Hz, 1H), 3.13 (dd, J=14.2, 7.6 Hz, 1H), 3.00 (dd, J=14.8, 7.8 Hz, 1H), 1.10-1.01 (m, 2H), 0.84-0.73 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 8% B to 3 8% B in 10 min; Wave Length: 254 nm/220 nm; RT1(min): 8.5.

### Synthetic Procedure of (2R,3S,4S)-4-hydroxy-2-[(4-propoxyphenyl)methyl]pyrrolidin-3-yl N-benzylcarbamate (V188376)

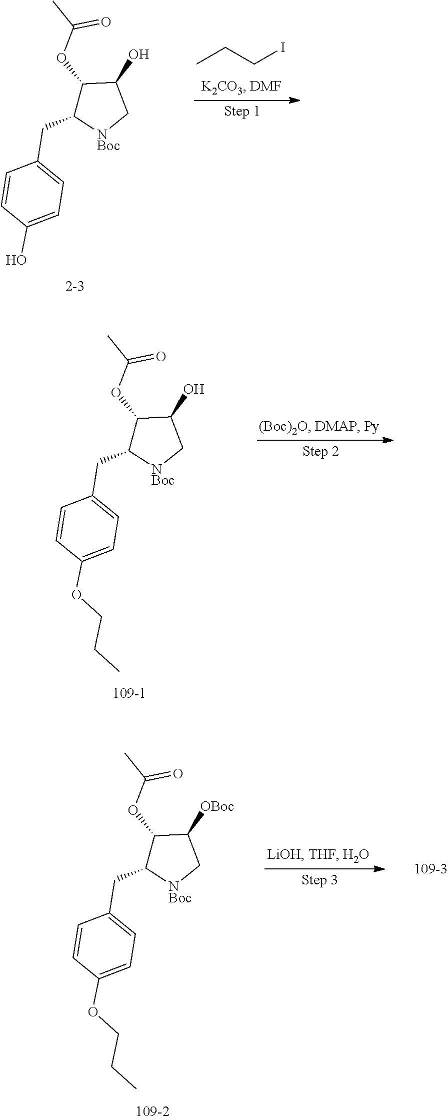

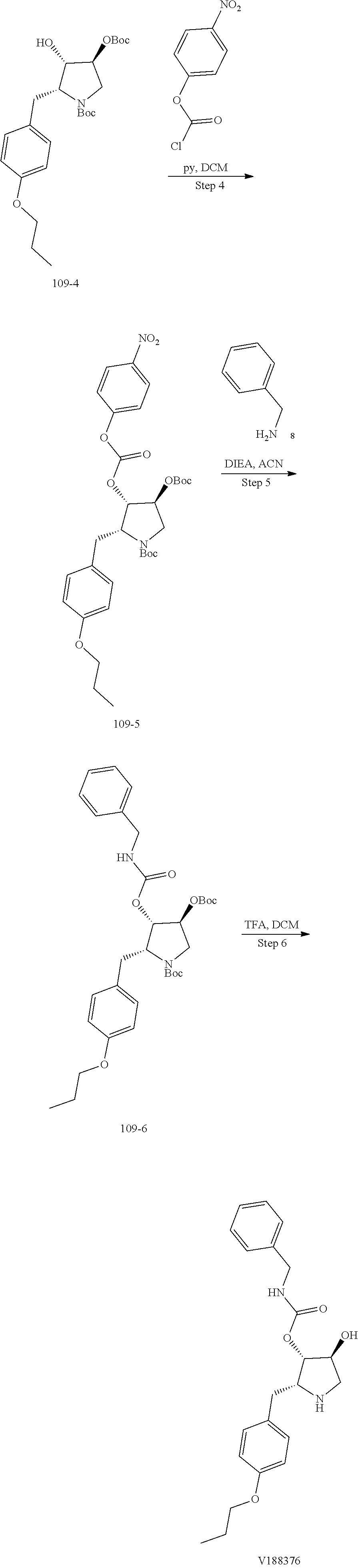

Step 1: To a stirred solution of tert-butyl (2R,3S,4S)-3-(acetyloxy)-4-hydroxy-2-[(4-hydroxyphenyl)methyl]pyrrolidine-1-carboxylate (2-4, 300 mg, 0.85 mmol, 1.0 eq.) and iodopropane (174.2 mg, 1.03 mmol, 1.2 eq.) in DMF (5 mL) was added K₂CO₃ (354 mg, 2.56 mmol, 3 eq.) in portions at room temperature. The resulting mixture was stirred over night at 25° C. Desired product could be detected by LCMS. The residue was purified by reversed-phase flash chromatography with the following conditions: column, C18 silica gel; mobile phase, MeCN in Water (0.1% TFA), 10% to 50% gradient in 10 min; detector, UV 220 nm. This resulted in tert-butyl (2R,3S,4S)-3-(acetyloxy)-4-hydroxy-2-[(4-propoxyphenyl)methyl]pyrrolidine-1-carboxylate (320 mg, 95.3%) as a white solid. MS: m/z: Calc'd for C₂₁H₃₁NO₆ [M+H−100]⁺294. Found, 294.

Step 2: To a stirred solution of tert-butyl (2R,3S,4S)-3-(acetyloxy)-4-hydroxy-2-[(4-propoxyphenyl)methyl]pyrrolidine-1-carboxylate (109-1, 340 mg, 0.864 mmol, 1 equiv) in Pyridine (20 mL) was added DMAP (158.4 mg, 1.30 mmol, 1.5 eq.) and Boc₂O (942.93 mg, 4.32 mmol, 5 eq.) in portions at 0° C. The resulting mixture was stirred over night at 25° C. The resulting mixture was concentrated under reduced pressure. The residue was purified by reversed-phase flash chromatography with the following conditions: column, C18 silica gel; mobile phase, MeCN in Water (0.1% NH₃·H₂O), 10% to 50% gradient in 10 min; detector, UV 220 nm. This resulted in tert-butyl (2R,3S,4S)-3-(acetyloxy)-4-[(tert-butoxycarbonyl)oxy]-2-[(4-propoxyphenyl)methyl]pyrrolidine-1-carboxylate (370 mg, 86.8%) as a light yellow oil. MS: m/z: Calc'd for C₂₆H₃₉NO₈ [M+H]⁺494. Found, 494.

Step 3: The compound was prepared in 91.6% yield as a white solid according to Hydrolysis reaction with LiOH; General Procedure V using tert-butyl (2R,3S,4S)-3-(acetyloxy)-4-[(tert-butoxycarbonyl)oxy]-2-[(4-propoxyphenyl)methyl]pyrrolidine-1-carboxylate (109-2) instead of tert-butyl (2R,3S,4S)-3-(acetyloxy)-4-[(tert-butoxycarbonyl)oxy]-2-[(4-fluorophenyl)methyl]pyrrolidine-1-carboxylate (3-1). MS: m/z Calc'd for C₂₄H₃₇NO₇ [M+H−56−56]⁺340. Found, 340.

Step 4: To a stirred solution of tert-butyl (2R,3S,4S)-4-[(tert-butoxycarbonyl)oxy]-3-hydroxy-2-[(4-propoxyphenyl)methyl]pyrrolidine-1-carboxylate (109-3, 320 mg, 0.71 mmol, 1 eq.) and 4-nitrophenyl carbonochloridate (214.3 mg, 1.06 mmol, 1.5 eq.) in DCM (10 mL) was added Pyridine (112.1 mg, 1.42 mmol, 2 eq.) in portions at room temperature. The resulting mixture was stirred over night at 25° C. Desired product could be detected by LCMS. The residue was purified by Prep-TLC (PE/EA 3:1) to afford tert-butyl (2R,3S,4S)-4-[(tert-butoxycarbonyl)oxy]-3-[(4-nitrophenoxycarbonyl)oxy]-2-[(4-propoxyphenyl)methyl]pyrrolidine-1-carboxylate (340 mg, 77.8%) as a white solid. MS: m/z: Calc'd for C₃₁H₄₀N₂O₁₁ [M+H−56−56]⁺505. Found, 505.

### Step 5 and 6

The title compound was prepared in 54.6% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-[(4-propoxyphenyl)methyl]pyrrolidine-1-carboxylate (109-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), benzylamine in STEP 5; Boc Deprotection; General Procedure II in STEP 6. MS: m/z Calc'd for C₂₂H₂₈N₂O₄ [M+H]⁺385. Found, 385. ¹H NMR (400 MHz, Methanol-d₄) δ 7.46-7.26 (m, 5H), 7.19 (d, J=8.3 Hz, 2H), 6.90-6.83 (m, 2H), 4.95 (d, J=3.5 Hz, 1H), 4.43-4.26 (m, 3H), 4.18-4.14 (m, 1H), 3.94-3.90 (m, 2H), 3.62-3.53 (m, 1H), 3.22 (d, J=12.6 Hz, 1H), 3.10 (dd, J=14.0, 7.5 Hz, 1H), 2.96 (dd, J=14.1, 8.2 Hz, 1H), 1.85-1.75 (m, 2H), 1.08-1.04 (m, 3H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 20% B to 50% B in 10 min; Wave Length: 254 nm/220 nm; RT1(min): 8.5.

### Synthetic Procedure of (2R,3S,4S)-4-hydroxy-2-{[4-(1-methyl-1,2,3-triazol-4-yl)phenyl]methyl}pyrrolidin-3-yl N-benzylcarbamate (V188392)

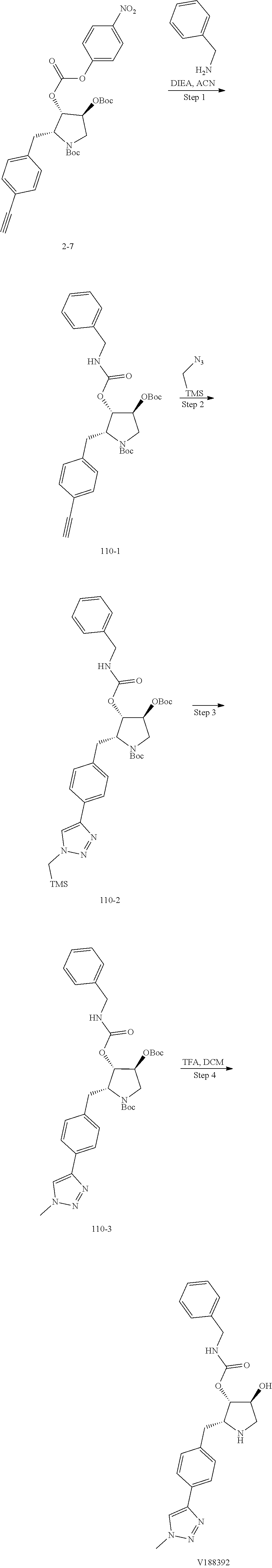

### Step 1 and 2

The compound was prepared in 76.7% yield as a light yellow oil according to Substitution reaction for synthesis of Carbamate; General Procedure I using tert-butyl (2R,3S,4S)-4-[(tert-butoxycarbonyl)oxy]-2-[(4-ethynylphenyl)methyl]-3-[(4-nitrophenoxycarbonyl)oxy]pyrrolidine-1-carboxylate (2-7) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), benzylamine in STEP 1; Cyclization reaction for synthesis of Substituted triazoles; General Procedure III using (azidomethyl)trimethylsilane in STEP 2. MS: m/z Calc'd for C₃₅H₄₉N₅O₇Si [M+H]⁺680. found 680.

Step 3: A solution of tert-butyl (2R,3S,4S)-3-[(benzylcarbamoyl)oxy]-4-[(tert-butoxycarbonyl)oxy]-2-[(4-{1-[(trimethylsilyl)methyl]-1,2,3-triazol-4-yl}phenyl)methyl]pyrrolidine-1-carboxylate (110-2, 210 mg, 0.31 mmol, 1 eq.) and TBAF (121.1 mg, 0.46 mmol, 1.5 eq.) in THF (10 mL) was stirred at 0° C. for 30 min under nitrogen atmosphere. After completion of reaction monitored by LCMS. The residue was purified by Prep-TLC (PE:EA, 1:2). This resulted in tert-butyl (2R,3S,4S)-3-[(benzylcarbamoyl)oxy]-4-[(tert-butoxycarbonyl)oxy]-2-{[4-(1-methyl-1,2,3-triazol-4-yl)phenyl]methyl}pyrrolidine-1-carboxylate (190 mg, 99.2%) as a colorless oil. MS: m/z: Calc'd for C₃₂H₄₁N₅O₇ [M+H]⁺ 608. found 608.

Step 4: The title compound was prepared in 19.1% yield as a white solid according to Boc Deprotection; General Procedure II. MS: m/z Calc'd for C₂₂H₂₅N₅O₃ [M+H]⁺408. found 408. ¹H NMR (400 MHz, Methanol-d₄) δ 8.28 (d, J=1.3 Hz, 1H), 7.80 (dd, J=8.2, 1.7 Hz, 2H), 7.42-7.25 (m, 7H), 4.99 (d, J=3.5 Hz, 1H), 4.45-4.22 (m, 4H), 4.17 (d, J=1.4 Hz, 3H), 3.66-3.57 (m, 1H), 3.29-3.17 (m, 2H), 3.14-3.04 (m, 1H).

Prep-HPLC purification conditions: Xselect CSH Prep C18 Column, 30*250 mm, 10 μm; Mobile Phase A: Water (0.05% TFA), Mobile Phase B: ACN; Flow rate: 60 mL/min; Gradient: 7% B to 37% B in 10 min; Wave Length: 254 nm/220 nm; RT1(min): 9.5.

### Synthetic Procedure of (2R,3S,4S)-2-({4-[1-(35-{5-[(3aS,4S,6aR)-2-oxo-hexahydrothieno[3,4-d]imidazol-4-yl]pentanamido}-3,6,9,12,15,18,21,24,27,30,33-undecaoxapentatriacontan-1-yl)-1,2,3-triazol-4-yl]phenyl}methyl)-4-hydroxypyrrolidin-3-yl N-benzylcarbamate (V190422)

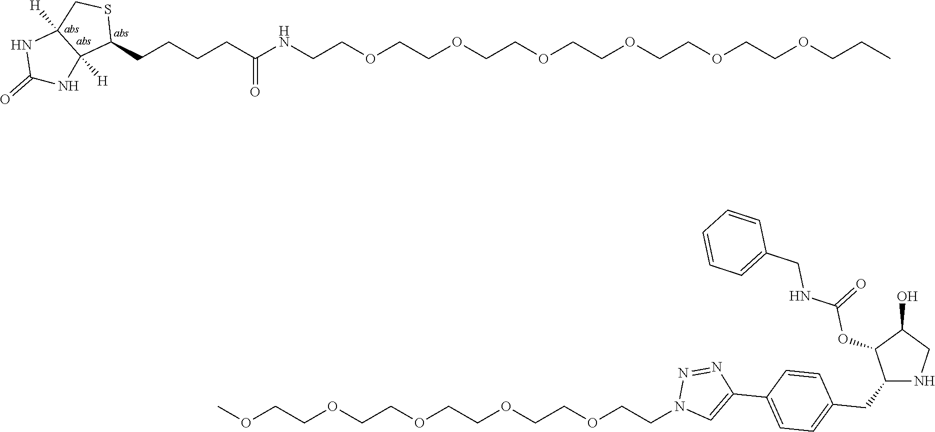

The title compound was prepared in 24.9% overall yield as a light yellow oil according to Cyclization reaction for synthesis of Substituted triazoles; General Procedure III using 5-[(3aS,4S,6aR)-2-oxo-hexahydrothieno[3,4-d]imidazol-4-yl]-N-(35-azido-3,6,9,12,15,18,21,24,27,30,33-undecaoxapentatriacontan-1-yl)pentanamide in STEP 1; Boc Deprotection; General Procedure II in STEP 2. MS: m/z Calc'd for C₅₅H₈₆N₈O₁₆S [M+H]⁺1148, found 1148. ¹H NMR (400 MHz, Methanol-d₄) δ 8.41 (s, 1H), 7.84 (d, J=8.0 Hz, 2H), 7.48-7.17 (m, 7H), 5.01 (d, J=3.5 Hz, 1H), 4.67 (t, J=4.9 Hz, 2H), 4.50 (dd, J=7.8, 4.9 Hz, 1H), 4.46-4.39 (m, 1H), 4.38 (s, 1H), 4.36-4.28 (m, 2H), 3.97 (t, J=5.0 Hz, 2H), 3.71-3.62 (m, 30H), 3.61-3.56 (m, 13H), 3.54 (d, J=5.5 Hz, 1H), 3.37 (d, J=5.5 Hz, 2H), 3.29-3.17 (m, 3H), 3.09 (dd, J=13.9, 8.3 Hz, 1H), 2.94 (dd, J=12.8, 5.0 Hz, 1H), 2.72 (d, J=12.8 Hz, 1H), 2.23 (t, J=7.4 Hz, 2H), 1.80-1.58 (m, 4H), 1.51-1.41 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 17% B to 27% B in 18 min; Wave Length: 254 nm/220 nm; RT1(min): 18.5.

### Synthetic Procedure of (2R,3S,4S)-2-(4-(difluoromethyl)benzyl)-4-hydroxypyrrolidin-3-yl (isothiazol-4-ylmethyl)carbamate (V192929)

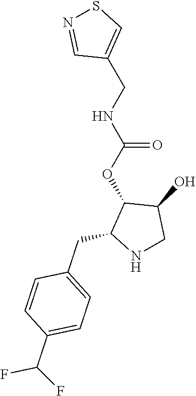 [SMILES: O=C(NCc1cnsc1)O[C@@H]1[C@@H](O)CN[C@@H]1Cc1ccc(C(F)F)cc1]

The title compound was prepared in 68.1% overall yield as a white solid according to Substitution reaction for synthesis of Carbamate; General Procedure I using tert-butyl (2R,3S,4S)-4-[(tert-butoxycarbonyl)oxy]-2-{[4-(difluoromethyl)phenyl]methyl}-3-[(4-nitrophenoxycarbonyl)oxy]pyrrolidine-1-carboxylate (85-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), isothiazol-4-ylmethanamine in STEP 1; Boc Deprotection; General Procedure II in STEP 2. MS: m/z: Calc'd for C₁₇H₁₉F₂N₃O₃S [M+H]⁺384. Found, 384.

¹H NMR (400 MHz, Methanol-d₄) δ 8.79 (s, 1H), 8.53 (s, 1H), 7.53 (d, J=7.9 Hz, 2H), 7.42 (d, J=7.9 Hz, 2H), 6.93-6.61 (m, 1H), 5.01-4.95 (m, 1H), 4.53-4.38 (m, 3H), 4.31-4.21 (m, 1H), 3.59 (dd, J=12.7, 4.3 Hz, 1H), 3.23 (dd, J=13.5, 7.8 Hz, 2H), 3.10 (dd, J=14.1, 8.4 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 6% B to 36% B in 10 min; Wave Length: 254 nm/220 nm; RT1(min): 8.5.

### Synthetic Procedure of (2R,3S,4S)-2-(4-(difluoromethyl)benzyl)-4-hydroxypyrrolidin-3-yl (2-(trifluoromethoxy)ethyl)carbamate (V192930)

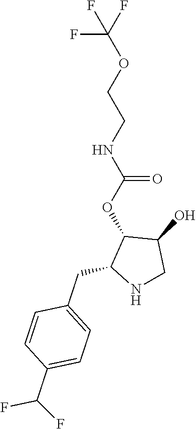 [SMILES: O=C(NCCOC(F)(F)F)O[C@@H]1[C@@H](O)CN[C@@H]1Cc1ccc(C(F)F)cc1]

The title compound was prepared in 51.1% overall yield as a white solid according to Substitution reaction for synthesis of Carbamate; General Procedure I using tert-butyl (2R,3S,4S)-4-[(tert-butoxycarbonyl)oxy]-2-{[4-(difluoromethyl)phenyl]methyl}-3-[(4-nitrophenoxycarbonyl)oxy]pyrrolidine-1-carboxylate (85-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 2-(trifluoromethoxy)ethan-1-amine in STEP 1; Boc Deprotection; General Procedure II in STEP 2. MS: m/z: Calc'd for C₁₆H₁₉F₅N₂O₄[M+H]⁺399. Found, 399.

¹H NMR (400 MHz, Methanol-d₄) δ 7.57 (d, J=7.9 Hz, 2H), 7.46 (d, J=8.0 Hz, 2H), 6.94-6.60 (m, 1H), 4.99 (d, J=3.6 Hz, 1H), 4.41 (d, J=4.1 Hz, 1H), 4.30-4.21 (m, 1H), 4.19-4.09 (m, 2H), 3.63-3.56 (m, 1H), 3.55-3.40 (m, 2H), 3.26 (dd, J=13.5, 7.6 Hz, 2H), 3.17-3.06 (m, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 13% B to 43% B in 10 min; Wave Length: 254 nm/220 nm; RT1(min): 8.5.

### Synthetic Procedure of (2R,3S,4S)-2-(4-(difluoromethyl)benzyl)-4-hydroxypyrrolidin-3-yl (2-((S)-pyrrolidin-3-yl)ethyl)carbamate (V193010)

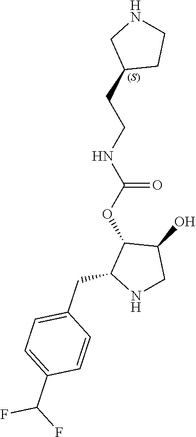

The title compound was prepared in 48.5% overall yield as a white solid according to Substitution reaction for synthesis of Carbamate; General Procedure I using tert-butyl (2R,3S,4S)-4-[(tert-butoxycarbonyl)oxy]-2-{[4-(difluoromethyl)phenyl]methyl}-3-[(4-nitrophenoxycarbonyl)oxy]pyrrolidine-1-carboxylate (85-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (R)-3-(2-aminoethyl)pyrrolidine-1-carboxylate in STEP 1; Boc Deprotection; General Procedure II in STEP 2. MS: m/z: Calc'd for C₁₉H₂₇F₂N₃O₃[M+H]⁺384. Found, 384. ¹H NMR (400 MHz, Methanol-d₄) δ 7.57 (d, J=7.9 Hz, 2H), 7.47 (d, J=7.9 Hz, 2H), 6.93-6.64 (m, 1H), 4.98 (d, J=3.6 Hz, 1H), 4.40 (d, J=4.2 Hz, 1H), 4.31-4.22 (m, 1H), 3.58 (dd, J=12.5, 4.2 Hz, 1H), 3.50 (d, J=19.0 Hz, 1H), 3.45-3.37 (m, 1H), 3.30-3.16 (m, 5H), 3.15-3.07 (m, 1H), 2.88 (dd, J=11.6, 9.1 Hz, 1H), 2.42-2.21 (m, 2H), 1.81-1.61 (m, 3H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.5.

### Synthetic Procedure of (2R,3S,4S)-4-hydroxy-2-(4-(oxazol-5-yl)benzyl)pyrrolidin-3-yl (2-((S)-pyrrolidin-3-yl)ethyl)carbamate (V193011)

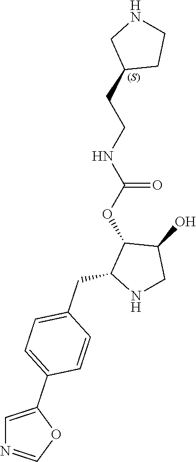

The title compound was prepared in 35.2% overall yield as an off-white semi-solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (R)-3-(2-aminoethyl)pyrrolidine-1-carboxylate in STEP 1; Boc Deprotection; General Procedure II in STEP 2. MS: m/z: Calc'd for C₂₁H₂₈N₄O₄ [M+H]⁺401. found 401. ¹H NMR (400 MHz, Methanol-d₄) δ 8.28 (s, 1H), 7.76 (d, J=8.2 Hz, 2H), 7.54 (s, 1H), 7.45 (d, J=8.2 Hz, 2H), 5.00 (d, J=3.6 Hz, 1H), 4.40 (d, J=4.1 Hz, 1H), 4.27 (s, 1H), 3.62-3.50 (m, 1H), 3.50-3.36 (m, 1H), 3.26 (d, J=6.3 Hz, 1H), 3.26-3.18 (m, 5H), 3.09 (dd, J=14.1, 8.6 Hz, 1H), 2.87 (dd, J=11.5, 9.1 Hz, 1H), 2.34 (dd, J=16.7, 7.9 Hz, 1H), 2.28 (s, 1H), 1.79-1.61 (m, 3H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.5.

### Synthetic Procedure of (2R,3S,4S)-4-hydroxy-2-(4-(oxazol-5-yl)benzyl)pyrrolidin-3-yl (2-(difluoromethoxy)ethyl)carbamate (V193066)

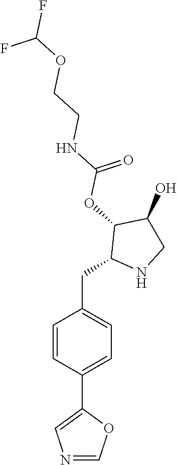 [SMILES: O=C(NCCOC(F)F)O[C@@H]1[C@@H](O)CN[C@@H]1Cc1ccc(-c2cnco2)cc1]

The title compound was prepared in 48.6% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 2-(difluoromethoxy)ethan-1-amine in STEP 1; Boc Deprotection; General Procedure II in STEP 2. MS: m/z: Calc'd for C₁₈H₂₁F₂N₃O₅[M+H]⁺398. found 398. ¹H NMR (400 MHz, Methanol-d₄) δ 8.27 (s, 1H), 7.80-7.73 (m, 2H), 7.54 (s, 1H), 7.44 (d, J=8.1 Hz, 2H), 6.45 (s, 1H), 4.99 (d, J=3.5 Hz, 1H), 4.41 (d, J=4.2 Hz, 1H), 4.26 (dd, J=7.9, 3.6 Hz, 1H), 3.96 (d, J=5.4 Hz, 2H), 3.58 (dd, J=12.6, 4.3 Hz, 1H), 3.43 (dd, J=9.1, 5.4 Hz, 2H), 3.29-3.18 (m, 2H), 3.08 (dd, J=14.1, 8.5 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 4% B to 34% B in 10 min; Wave Length: 254 nm/220 nm; RT1(min): 8.5

### Synthetic Procedure of (2R,3S,4S)-4-hydroxy-2-(4-(oxazol-5-yl)benzyl)pyrrolidin-3-yl (3-(difluoromethoxy)benzyl)carbamate (V193068)

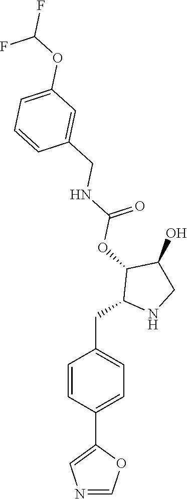 [SMILES: O=C(NCc1cccc(OC(F)F)c1)O[C@@H]1[C@@H](O)CN[C@@H]1Cc1ccc(-c2cnco2)cc1]

The title compound was prepared in 43.5% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (3-(difluoromethoxy)phenyl)methanamine in STEP 1; Boc Deprotection; General Procedure II in STEP 2. MS: m/z: Calc'd for C₂₃H₂₃F₂N₃O₅[M+H]⁺460. found 460.

¹H NMR (400 MHz, Methanol-d₄) δ 8.27 (s, 1H), 7.75-7.68 (m, 2H), 7.53 (s, 1H), 7.40 (dd, J=8.0, 3.6 Hz, 3H), 7.23 (t, J=10.0 Hz, 1H), 7.15 (s, 1H), 7.10 (d, J=8.4 Hz, 1H), 6.84 (s, 1H), 4.99 (d, J=3.5 Hz, 1H), 4.41 (d, J=15.7 Hz, 3H), 4.36-4.22 (m, 1H), 3.60 (dd, J=12.5, 4.3 Hz, 1H), 3.33-3.26 (m, 1H), 3.23-3.12 (m, 1H), 3.09 (dd, J=14.0, 8.3 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 8.5.

### Synthetic Procedure of (2R,3S,4S)-4-hydroxy-2-(4-(oxazol-5-yl)benzyl)pyrrolidin-3-yl (3-fluorophenethyl)carbamate (V193069)

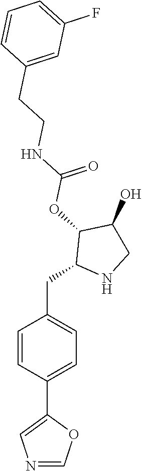 [SMILES: O=C(NCCc1cccc(F)c1)O[C@@H]1[C@@H](O)CN[C@@H]1Cc1ccc(-c2cnco2)cc1]

The title compound was prepared in 44.2% overall yield as an off-white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 2-(3-fluorophenyl)ethan-1-amine in STEP 1; Boc Deprotection; General Procedure II in STEP 2. MS: m/z: Calc'd for C23H₂₄FN₃O₄[M+H]⁺426. found 426. ¹H NMR (400 MHz, Methanol-d₄) δ 8.27 (s, 1H), 7.78-7.71 (m, 2H), 7.54 (s, 1H), 7.40 (d, J=8.3 Hz, 2H), 7.37-7.27 (m, 1H), 7.08 (d, J=7.6 Hz, 1H), 7.02 (d, J=10.1 Hz, 1H), 6.95 (dd, J=8.6, 2.7 Hz, 1H), 4.96 (d, J=3.6 Hz, 1H), 4.35 (d, J=4.2 Hz, 1H), 4.22 (dd, J=7.9, 3.4 Hz, 1H), 3.54 (dd, J=12.6, 4.3 Hz, 1H), 3.43 (d, J=7.0 Hz, 2H), 3.26-3.23 (m, 1H), 3.19-3.11 (m, 1H), 3.00 (dd, J=14.2, 8.6 Hz, 1H), 2.88 (d, J=6.9 Hz, 2H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 8.5.

### Synthetic Procedure of (2R,3S,4S)-4-hydroxy-2-(4-(oxazol-5-yl)benzyl)pyrrolidin-3-yl (3,5-difluorobenzyl)carbamate (V193071)

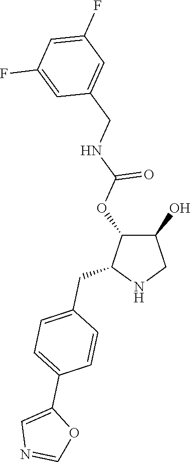 [SMILES: O=C(NCc1cc(F)cc(F)c1)O[C@@H]1[C@@H](O)CN[C@@H]1Cc1ccc(-c2cnco2)cc1]

The title compound was prepared in 49.4% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (3,5-difluorophenyl)methanamine in STEP 1; Boc Deprotection; General Procedure II in STEP 2. MS: m/z: Calc'd for C₂₂H₂₁F₂N₃O₄[M+H]⁺430. found 430. ¹H NMR (400 MHz, Methanol-d₄) δ 8.28 (s, 1H), 7.77-7.69 (m, 2H), 7.53 (s, 1H), 7.42 (d, J=8.1 Hz, 2H), 7.00-6.90 (m, 3H), 4.98 (d, J=3.6 Hz, 1H), 4.46-4.36 (m, 3H), 4.35-4.24 (m, 1H), 3.61 (dd, J=12.6, 4.2 Hz, 1H), 3.31-3.18 (m, 2H), 3.11 (dd, J=14.0, 8.3 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 12% B to 42% B in 10 min; Wave Length: 254 nm/220 nm; RT1(min): 8.5.

### Synthetic Procedure of (2R,3S,4S)-2-(4-(difluoromethyl)benzyl)-4-hydroxypyrrolidin-3-yl (isothiazol-4-ylmethyl)carbamate (V192929)

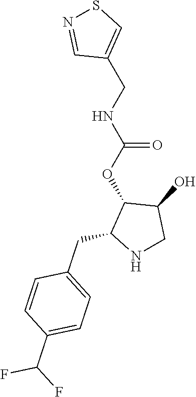 [SMILES: O=C(NCc1cnsc1)O[C@@H]1[C@@H](O)CN[C@@H]1Cc1ccc(C(F)F)cc1]

The title compound was prepared in 68.1% overall yield as a white solid according to Substitution reaction for synthesis of Carbamate; General Procedure I using tert-butyl (2R,3S,4S)-4-[(tert-butoxycarbonyl)oxy]-2-{[4-(difluoromethyl)phenyl]methyl}-3-[(4-nitrophenoxycarbonyl)oxy]pyrrolidine-1-carboxylate (85-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), isothiazol-4-ylmethanamine in STEP 1; Boc Deprotection; General Procedure II in STEP 2. MS: m/z: Calc'd for C₁₇H₁₉F₂N₃O₃S [M+H]⁺384. Found, 384.

¹H NMR (400 MHz, Methanol-d₄) δ 8.79 (s, 1H), 8.53 (s, 1H), 7.53 (d, J=7.9 Hz, 2H), 7.42 (d, J=7.9 Hz, 2H), 6.93-6.61 (m, 1H), 5.01-4.95 (m, 1H), 4.53-4.38 (m, 3H), 4.31-4.21 (m, 1H), 3.59 (dd, J=12.7, 4.3 Hz, 1H), 3.23 (dd, J=13.5, 7.8 Hz, 2H), 3.10 (dd, J=14.1, 8.4 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min n; Gradient: 6% B to 36% B in 10 min; Wave Length: 254 nm/220 nm; RT1(min): 8.5.

### Synthetic Procedure of (2R,3S,4S)-2-(4-(difluoromethyl)benzyl)-4-hydroxypyrrolidin-3-yl (2-(trifluoromethoxy)ethyl)carbamate (V192930)

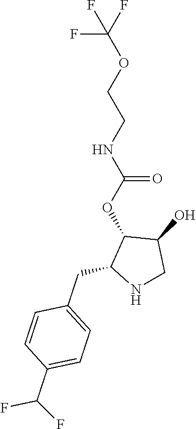 [SMILES: O=C(NCCOC(F)(F)F)O[C@@H]1[C@@H](O)CN[C@@H]1Cc1ccc(C(F)F)cc1]

The title compound was prepared in 51.1% overall yield as a white solid according to Substitution reaction for synthesis of Carbamate; General Procedure I using tert-butyl (2R,3S,4S)-4-[(tert-butoxycarbonyl)oxy]-2-{[4-(difluoromethyl)phenyl]methyl}-3-[(4-nitrophenoxycarbonyl)oxy]pyrrolidine-1-carboxylate (85-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 2-(trifluoromethoxy)ethan-1-amine in STEP 1; Boc Deprotection; General Procedure II in STEP 2. MS: m/z: Calc'd for C₁₆H₁₉F₅N₂O₄[M+H]⁺399. Found, 399.

¹H NMR (400 MHz, Methanol-d₄) δ 7.57 (d, J=7.9 Hz, 2H), 7.46 (d, J=8.0 Hz, 2H), 6.94-6.60 (m, 1H), 4.99 (d, J=3.6 Hz, 1H), 4.41 (d, J=4.1 Hz, 1H), 4.30-4.21 (m, 1H), 4.19-4.09 (m, 2H), 3.63-3.56 (m, 1H), 3.55-3.40 (m, 2H), 3.26 (dd, J=13.5, 7.6 Hz, 2H), 3.17-3.06 (m, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 13% B to 43% B in 10 min; Wave Length: 254 nm/220 nm; RT1(min): 8.5.

### Synthetic Procedure of (2R,3S,4S)-2-(4-(difluoromethyl)benzyl)-4-hydroxypyrrolidin-3-yl (2-((S)-pyrrolidin-3-yl)ethyl)carbamate (V193010)

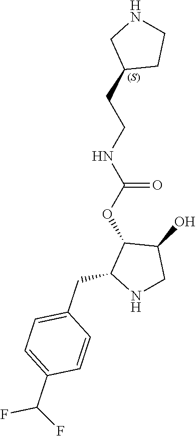

The title compound was prepared in 48.5% overall yield as a white solid according to Substitution reaction for synthesis of Carbamate; General Procedure I using tert-butyl (2R,3S,4S)-4-[(tert-butoxycarbonyl)oxy]-2-{[4-(difluoromethyl)phenyl]methyl}-3-[(4-nitrophenoxycarbonyl)oxy]pyrrolidine-1-carboxylate (85-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (R)-3-(2-aminoethyl)pyrrolidine-1-carboxylate in STEP 1; Boc Deprotection; General Procedure II in STEP 2. MS: m/z: Calc'd for C₁₉H₂₇F₂N₃O₃[M+H]⁺384. Found, 384. ¹H NMR (400 MHz, Methanol-d₄) δ 7.57 (d, J=7.9 Hz, 2H), 7.47 (d, J=7.9 Hz, 2H), 6.93-6.64 (m, 1H), 4.98 (d, J=3.6 Hz, 1H), 4.40 (d, J=4.2 Hz, 1H), 4.31-4.22 (m, 1H), 3.58 (dd, J=12.5, 4.2 Hz, 1H), 3.50 (d, J=19.0 Hz, 1H), 3.45-3.37 (m, 1H), 3.30-3.16 (m, 5H), 3.15-3.07 (m, 1H), 2.88 (dd, J=11.6, 9.1 Hz, 1H), 2.42-2.21 (m, 2H), 1.81-1.61 (m, 3H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 10 min; Wave Length: 254 nm/220 nm; RT1(min): 8.5.

### Synthetic Procedure of: (2R,3S,4S)-4-hydroxy-2-(4-(oxazol-5-yl)benzyl)pyrrolidin-3-yl (2-((S)-pyrrolidin-3-yl)ethyl)carbamate (V193011)

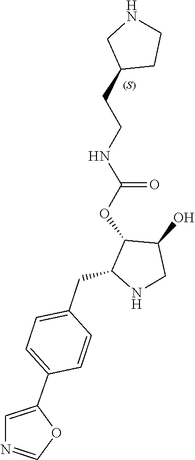

The title compound was prepared in 35.2% overall yield as an off-white semi-solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (R)-3-(2-aminoethyl)pyrrolidine-1-carboxylate in STEP 1; Boc Deprotection; General Procedure II in STEP 2. MS: m/z: Calc'd for C₂₁H₂₈N₄O₄[M+H]⁺401. found 401. ¹H NMR (400 MHz, Methanol-d₄) δ 8.28 (s, 1H), 7.76 (d, J=8.2 Hz, 2H), 7.54 (s, 1H), 7.45 (d, J=8.2 Hz, 2H), 5.00 (d, J=3.6 Hz, 1H), 4.40 (d, J=4.1 Hz, 1H), 4.27 (s, 1H), 3.62-3.50 (m, 1H), 3.50-3.36 (m, 1H), 3.26 (d, J=6.3 Hz, 1H), 3.26-3.18 (m, 5H), 3.09 (dd, J=14.1, 8.6 Hz, 1H), 2.87 (dd, J=11.5, 9.1 Hz, 1H), 2.34 (dd, J=16.7, 7.9 Hz, 1H), 2.28 (s, 1H), 1.79-1.61 (m, 3H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min mL/min; Gradient: 2% B to 22% B in 10 min; Wave Length: 254 nm/220 nm; RT1(min): 8.5.

### Synthetic Procedure of (2R,3S,4S)-4-hydroxy-2-(4-(oxazol-5-yl)benzyl)pyrrolidin-3-yl (2-(difluoromethoxy)ethyl)carbamate (V193066)

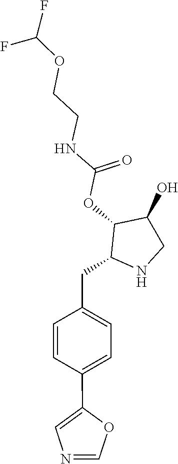 [SMILES: O=C(NCCOC(F)F)O[C@@H]1[C@@H](O)CN[C@@H]1Cc1ccc(-c2cnco2)cc1]

The title compound was prepared in 48.6% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 2-(difluoromethoxy)ethan-1-amine in STEP 1; Boc Deprotection; General Procedure II in STEP 2. MS: m/z: Calc'd for C₁₈H₂₁F₂N₃O₅[M+H]⁺398. found 398. ¹H NMR (400 MHz, Methanol-d₄) δ 8.27 (s, 1H), 7.80-7.73 (m, 2H), 7.54 (s, 1H), 7.44 (d, J=8.1 Hz, 2H), 6.45 (s, 1H), 4.99 (d, J=3.5 Hz, 1H), 4.41 (d, J=4.2 Hz, 1H), 4.26 (dd, J=7.9, 3.6 Hz, 1H), 3.96 (d, J=5.4 Hz, 2H), 3.58 (dd, J=12.6, 4.3 Hz, 1H), 3.43 (dd, J=9.1, 5.4 Hz, 2H), 3.29-3.18 (m, 2H), 3.08 (dd, J=14.1, 8.5 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 4% B to 34% B in 10 min; Wave Length: 254 nm/220 nm; RT1(min): 8.5.

### Synthetic Procedure of (2R,3S,4S)-4-hydroxy-2-(4-(oxazol-5-yl)benzyl)pyrrolidin-3-yl (3-(difluoromethoxy)benzyl)carbamate (V193068)

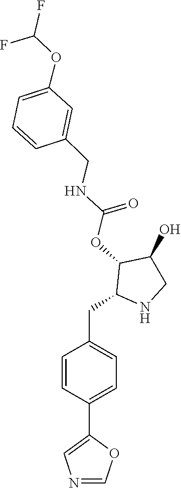 [SMILES: O=C(NCc1cccc(OC(F)F)c1)O[C@@H]1[C@@H](O)CN[C@@H]1Cc1ccc(-c2cnco2)cc1]

The title compound was prepared in 43.5% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (3-(difluoromethoxy)phenyl)methanamine in STEP 1; Boc Deprotection; General Procedure II in STEP 2. MS: m/z: Calc'd for C₂₃H₂₃F₂N₃O₅[M+H]⁺460. found 460.

¹H NMR (400 MHz, Methanol-d₄) δ 8.27 (s, 1H), 7.75-7.68 (m, 2H), 7.53 (s, 1H), 7.40 (dd, J=8.0, 3.6 Hz, 3H), 7.23 (t, J=10.0 Hz, 1H), 7.15 (s, 1H), 7.10 (d, J=8.4 Hz, 1H), 6.84 (s, 1H), 4.99 (d, J=3.5 Hz, 1H), 4.41 (d, J=15.7 Hz, 3H), 4.36-4.22 (m, 1H), 3.60 (dd, J=12.5, 4.3 Hz, 1H), 3.33-3.26 (m, 1H), 3.23-3.12 (m, 1H), 3.09 (dd, J=14.0, 8.3 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 8.5.

### Synthetic Procedure of (2R,3S,4S)-4-hydroxy-2-(4-(oxazol-5-yl)benzyl)pyrrolidin-3-yl (3-fluorophenethyl)carbamate (V193069)

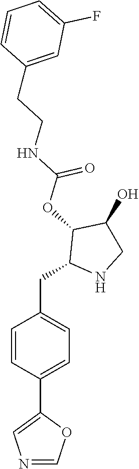 [SMILES: O=C(NCCc1cccc(F)c1)O[C@@H]1[C@@H](O)CN[C@@H]1Cc1ccc(-c2cnco2)cc1]

The title compound was prepared in 44.2% overall yield as an off-white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 2-(3-fluorophenyl)ethan-1-amine in STEP 1; Boc Deprotection; General Procedure II in STEP 2. MS: m/z: Calc'd for C₂₃H₂₄FN₃O₄[M+H]⁺426. found 426. ¹H NMR (400 MHz, Methanol-d₄) δ 8.27 (s, 1H), 7.78-7.71 (m, 2H), 7.54 (s, 1H), 7.40 (d, J=8.3 Hz, 2H), 7.37-7.27 (m, 1H), 7.08 (d, J=7.6 Hz, 1H), 7.02 (d, J=10.1 Hz, 1H), 6.95 (dd, J=8.6, 2.7 Hz, 1H), 4.96 (d, J=3.6 Hz, 1H), 4.35 (d, J=4.2 Hz, 1H), 4.22 (dd, J=7.9, 3.4 Hz, 1H), 3.54 (dd, J=12.6, 4.3 Hz, 1H), 3.43 (d, J=7.0 Hz, 2H), 3.26-3.23 (m, 1H), 3.19-3.11 (m, 1H), 3.00 (dd, J=14.2, 8.6 Hz, 1H), 2.88 (d, J=6.9 Hz, 2H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 8.5

### Synthetic Procedure of (2R,3S,4S)-4-hydroxy-2-(4-(oxazol-5-yl)benzyl)pyrrolidin-3-yl (3,5-difluorobenzyl)carbamate (V193071)

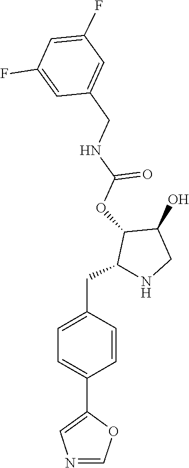 [SMILES: O=C(NCc1cc(F)cc(F)c1)O[C@@H]1[C@@H](O)CN[C@@H]1Cc1ccc(-c2cnco2)cc1]

The title compound was prepared in 49.4% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (3,5-difluorophenyl)methanamine in STEP 1; Boc Deprotection; General Procedure II in STEP 2. MS: m/z: Calc'd for C₂₂H₂₁F₂N₃O₄[M+H]⁺430. found 430. ¹H NMR (400 MHz, Methanol-d₄) δ 8.28 (s, 1H), 7.77-7.69 (m, 2H), 7.53 (s, 1H), 7.42 (d, J=8.1 Hz, 2H), 7.00-6.90 (m, 3H), 4.98 (d, J=3.6 Hz, 1H), 4.46-4.36 (m, 3H), 4.35-4.24 (m, 1H), 3.61 (dd, J=12.6, 4.2 Hz, 1H), 3.31-3.18 (m, 2H), 3.11 (dd, J=14.0, 8.3 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 12% B to 42% B in 10 min; Wave Length: 254 nm/220 nm; RT1(min): 8.5.

### Synthetic Procedure of (2R,3S,4S)-4-hydroxy-2-((4′-(pentafluoro-l6-sulfanyl)-[1,1′-biphenyl]-4-yl)methyl)pyrrolidin-3-yl (2-((S)-pyrrolidin-2-yl)ethyl)carbamate (V191968)

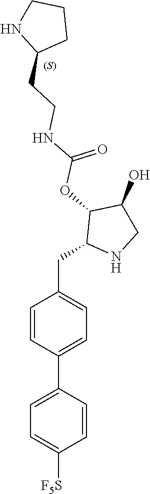

The title compound was prepared in 71.1% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4′-(pentafluoro-lambda6-sulfanyl)-[1,1′-biphenyl]-4-yl]methyl}pyrrolidine-1-carboxylate (62-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (S)-2-(2-aminoethyl)pyrrolidine-1-carboxylate in STEP 1; Boc Deprotection; General Procedure II in STEP 2. MS: m/z: Calc'd for C₂₄H₃₀F₅N₃O₃S [M+H]⁺536. Found, 536. ¹H NMR (400 MHz, Methanol-d₄) δ 7.92 (d, J=8.9 Hz, 2H), 7.82 (d, J=8.6 Hz, 2H), 7.73 (dd, J=8.1, 1.5 Hz, 2H), 7.48 (d, J=8.2 Hz, 2H), 5.01 (d, J=3.6 Hz, 1H), 4.42 (d, J=4.2 Hz, 1H), 4.33-4.24 (m, 1H), 3.64-3.54 (m, 2H), 3.32-3.21 (m, 6H), 3.15 (d, J=13.6 Hz, 1H), 2.35-2.23 (m, 1H), 2.16-1.87 (m, 4H), 1.76-1.66 (m, 1H).

Prep-HPLC-conditions: Column: Welch Ultimate PFP-C18 30*250, 10 μm; Mobile Phase A: Water (0.05% TFA), Mobile Phase B: ACN; Flow rate: 60 mL/min; Gradient: 10% B to 40% B in 10 min; Wave Length: 254 nm/220 nm; RT1(min): 10.48.

### Synthetic Procedure of (2R,3S,4S)-4-hydroxy-2-(4-methoxybenzyl)pyrrolidin-3-yl (2-(1-aminocyclopropyl)ethyl)carbamate (V191989)

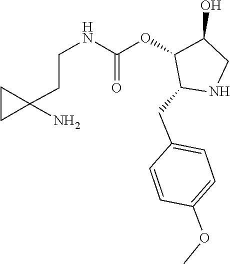 [SMILES: COc1ccc(C[C@H]2NC[C@H](O)[C@H]2OC(=O)NCCC2(N)CC2)cc1]

The title compound was prepared in 49.1% overall yield as a light grey solid according to Substitution reaction for synthesis of Carbamate; General Procedure I using tert-butyl (1-(2-aminoethyl)cyclopropyl)carbamate in STEP 1; Boc Deprotection; General Procedure II in STEP 2. MS: m/z: Calc'd for C₁₈H₂₇N₃O₄ [M+H]⁺350. found [M+H]⁺350. ¹H NMR (400 MHz, Methanol-d4) δ 7.25 (d, J=8.3 Hz, 2H), 6.93 (d, J=8.3 Hz, 2H), 4.98 (d, J=3.5 Hz, 1H), 4.40 (d, J=4.1 Hz, 1H), 4.23-4.13 (m, 1H), 3.80 (s, 3H), 3.59 (dd, J=12.6, 4.4 Hz, 1H), 3.43-3.35 (m, 2H), 3.23 (d, J=12.7 Hz, 1H), 3.12 (dd, J=14.0, 7.2 Hz, 1H), 2.99 (dd, J=14.2, 8.5 Hz, 1H), 1.90 (t, J=7.1 Hz, 2H), 0.99-0.82 (m, 4H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 8.5.

### Synthetic Procedure of (2R,3S,4S)-4-hydroxy-2-(4-(thiazol-5-yl)benzyl)pyrrolidin-3-yl ((1,2,3,4-tetrahydroisoquinolin-6-yl)methyl)carbamate (V192109)

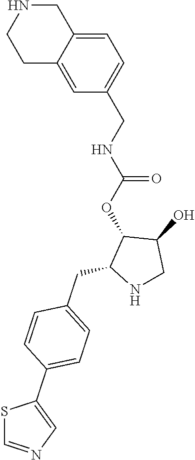 [SMILES: O=C(NCc1ccc2c(c1)CCNC2)O[C@@H]1[C@@H](O)CN[C@@H]1Cc1ccc(-c2cncs2)cc1]

The title compound was prepared in 48.1% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-thiazol-5-yl)phenyl]methyl}pyrrolidine-1-carboxylate (Int-13) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 6-(aminomethyl)-3,4-dihydroisoquinoline-2(1H)-carboxylate in STEP 1; Boc Deprotection; General Procedure II in STEP 2. MS: m/z: Calc'd for C₂₅H₂₈N₄O₃S [M+H]¹465, found [M+H]⁺465. ¹H NMR (400 MHz, Methanol-d4) δ 9.00 (s, 1H), 8.18 (s, 1H), 7.70-7.63 (m, 2H), 7.41 (d, J=8.0 Hz, 2H), 7.28 (d, J=8.0 Hz, 1H), 7.23 (d, J=7.3 Hz, 2H), 5.03-4.98 (m, 1H), 4.42 (d, J=4.1 Hz, 1H), 4.37 (s, 3H), 4.32 (s, 1H), 4.30-4.24 (m, 1H), 3.61 (dd, J=12.6, 4.3 Hz, 1H), 3.51 (t, J=6.3 Hz, 2H), 3.28 (s, 1H), 3.25-3.20 (m, 1H), 3.17-3.06 (m, 3H).

Prep-HPLC-conditions: Column: XBridge Prep Shield RP C18 Column, 30*150 mm, 5 μm; Mobile Phase A: Water (0.05% TFA), Mobile Phase B: ACN; Flow rate: 60 mL/min; Gradient: 3% B to 29% B in 7 min; Wave Length: 254 nm/220 nm; RT1(min): 6.28.

### Synthetic Procedure of (2R,3S,4S)-4-hydroxy-2-(4-(oxazol-5-yl)benzyl)pyrrolidin-3-yl (isothiazol-4-ylmethyl)carbamate (V192110)

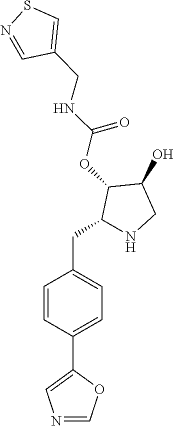 [SMILES: O=C(NCc1cnsc1)O[C@@H]1[C@@H](O)CN[C@@H]1Cc1ccc(-c2cnco2)cc1]

The title compound was prepared in 35.5% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), isothiazol-4-ylmethanamine in STEP 1; Boc Deprotection; General Procedure II in STEP 2. MS: m/z: Calc'd for C₁₉H₂₀N₄O₄S [M+H]⁺401. Found, 401. ¹H NMR (400 MHz, Methanol-d₄) δ 8.79 (s, 1H), 8.54 (s, 1H), 8.28 (s, 1H), 7.71 (d, J=8.1 Hz, 2H), 7.54 (s, 1H), 7.39 (d, J=8.0 Hz, 2H), 4.98 (d, J=3.5 Hz, 1H), 4.53-4.37 (m, 3H), 4.29-4.25 (m, J=7.9, 3.6 Hz, 1H), 3.60 (dd, J=12.8, 4.2 Hz, 1H), 3.29-3.00 (m, 3H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 8% B to 38% B in 10 min; Wave Length: 254 nm/220 nm; RT1(min): 6.9.

### Synthetic Procedure of (2R,3S,4S)-4-hydroxy-2-(4-(oxazol-5-yl)benzyl)pyrrolidin-3-yl ((2-(azetidin-1-yl)pyridin-4-yl)methyl)carbamate (V192160)

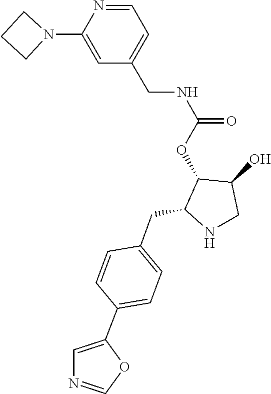 [SMILES: O=C(NCc1ccnc(N2CCC2)c1)O[C@@H]1[C@@H](O)CN[C@@H]1Cc1ccc(-c2cnco2)cc1]

The title compound was prepared in 27.4% overall yield as a light yellow solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (2-(azetidin-1-yl)pyridin-4-yl)methanamine in STEP 1; Boc Deprotection; General Procedure II in STEP 2. MS: m/z: Calc'd for C₂₄H₂₇N₅O₄ [M+H]⁺450. Found, 450. ¹H NMR (400 MHz, Methanol-d₄) δ 8.28 (s, 1H), 7.84 (d, J=6.6 Hz, 1H), 7.75 (d, J=8.0 Hz, 2H), 7.55 (s, 1H), 7.45 (d, J=8.0 Hz, 2H), 6.85 (dd, J=6.7, 1.6 Hz, 1H), 6.69 (s, 1H), 5.04 (d, J=3.6 Hz, 1H), 4.42 (dd, J=14.0, 4.5 Hz, 3H), 4.35-4.27 (m, 5H), 3.64 (dd, J=12.6, 4.2 Hz, 1H), 3.31-3.20 (m, 2H), 3.13 (dd, J=14.1, 8.5 Hz, 1H), 2.61-2.54 (m, 2H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 24% B in 10 min; Wave Length: 254 nm/220 nm; RT1(min): 8.5.

### Synthetic Procedure of: (2R,3S,4S)-4-hydroxy-2-(4-(oxazol-5-yl)benzyl)pyrrolidin-3-yl (2,2-difluoroethyl)carbamate (V192264)

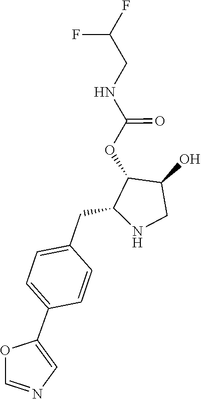 [SMILES: O=C(NCC(F)F)O[C@@H]1[C@@H](O)CN[C@@H]1Cc1ccc(-c2cnco2)cc1]

The title compound was prepared in 25.3% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 2,2-difluoroethane-1-amine in STEP 1; Boc Deprotection; General Procedure II in STEP 2. MS: m/z: Calc'd for C₁₇H₁₉F₂N₃O₄[M+H]⁺368. Found, 368. ¹H NMR (400 MHz, Methanol-d₄) δ 8.27 (s, 1H), 7.76 (d, J=8.1 Hz, 2H), 7.54 (s, 1H), 7.48-7.41 (m, 2H), 6.10-5.80 (m, 1H), 5.00 (d, J=3.5 Hz, 1H), 4.42 (d, J=4.3 Hz, 1H), 4.29-4.25 (m, 1H), 3.66-3.48 (m, 3H), 3.30-3.19 (m, 2H), 3.11 (dd, J=14.1, 8.4 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 18% B in 18 min; Wave Length: 254 nm/220 nm; RT1(min): 17.5.

### Synthetic Procedure of (2R,3S,4S)-4-hydroxy-2-(4-(oxazol-5-yl)benzyl)pyrrolidin-3-yl (3-(trifluoromethoxy)benzyl)carbamate (V192276)

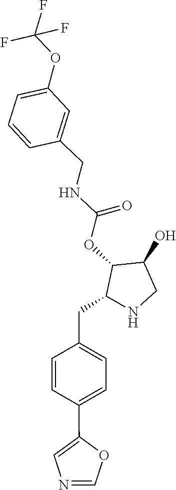 [SMILES: O=C(NCc1cccc(OC(F)(F)F)c1)O[C@@H]1[C@@H](O)CN[C@@H]1Cc1ccc(-c2cnco2)cc1]

The title compound was prepared in 49.4% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (3-(trifluoromethoxy)phenyl)methanamine in STEP 1; Boc Deprotection; General Procedure II in STEP 2. MS: m/z: Calc'd for C₂₃H₂₂F₃N₃O₅[M+H]⁺478. found 478.

¹H NMR (400 MHz, Methanol-d₄) δ 8.28 (s, 1H), 7.75-7.68 (m, 2H), 7.53 (s, 1H), 7.48 (d, J=7.9 Hz, 1H), 7.41-7.36 (d, J=8.1 Hz, 3H), 7.28 (s, 1H), 7.24 (d, J=8.1 Hz, 1H), 5.00 (d, J=3.5 Hz, 1H), 4.48-4.35 (m, 3H), 4.27 (dd, J=7.8, 3.5 Hz, 1H), 3.60 (dd, J=12.6, 4.3 Hz, 1H), 3.24 (dd, J=21.5, 10.0 Hz, 2H), 3.10 (dd, J=14.0, 8.3 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min mL/min; Gradient: 17% B to 47% B in 10 min; Wave Length: 254 nm/220 nm; RT1(min): 8.5.

### Synthetic Procedure of (2R,3S,4S)-2-(4-(difluoromethyl)benzyl)-4-hydroxypyrrolidin-3-yl (2-((S)-pyrrolidin-2-yl)ethyl)carbamate (V192893)

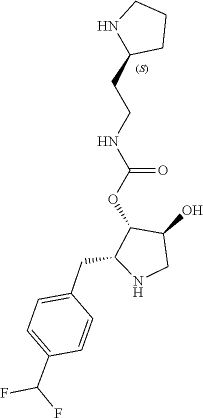

The title compound was prepared in 56.5% overall yield as an off-white solid according to Substitution reaction for synthesis of Carbamate; General Procedure I using tert-butyl (2R,3S,4S)-4-[(tert-butoxycarbonyl)oxy]-2-{[4-(difluoromethyl)phenyl]methyl}-3-[(4-nitrophenoxycarbonyl)oxy]pyrrolidine-1-carboxylate (85-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (S)-2-(2-aminoethyl)pyrrolidine-1-carboxylate in STEP 1; Boc Deprotection; General Procedure II in STEP 2. MS: m/z: Calc'd for C₁₉H₂₇F₂N₃O₃[M+H]⁺384. Found, 384. ¹H NMR (400 MHz, Methanol-d₄) δ 7.61-7.54 (m, 2H), 7.48 (d, J=8.0 Hz, 2H), 6.93-6.65 (m, 1H), 4.99 (s, 1H), 4.40 (d, J=4.1 Hz, 1H), 4.26 (d, J=8.1 Hz, 1H), 3.61-3.54 (m, 2H), 3.32-3.20 (m, 6H), 3.18-3.08 (m, 1H), 2.35-2.27 (m, 1H), 2.15-1.88 (m, 4H), 1.77-1.67 (m, 1H).

Prep-HPLC-conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 2% B to 32% B in 13 min; Wave Length: 254 nm/220 nm; RT1(min): 4.67.

### Synthetic Procedure of (2R,3S,4S)-4-hydroxy-2-[(4-methoxyphenyl)methyl]pyrrolidin-3-yl N-[(2-fluorophenyl)methyl]carbamate (V190520)

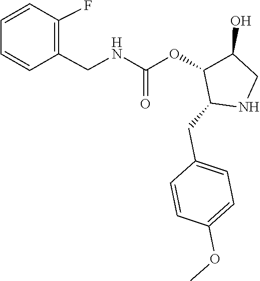 [SMILES: COc1ccc(C[C@H]2NC[C@H](O)[C@H]2OC(=O)NCc2ccccc2F)cc1]

The title compound was prepared in 59.7% overall yield as a white solid according to Substitution reaction for synthesis of Carbamate; General Procedure I using 1-(2-fluorophenyl)methanamine in STEP 1; Boc Deprotection; General Procedure II in STEP 2. MS: m/z: Calc'd for C₂₀H₂₃FN₂O₄[M+H]⁺375. found 375.

¹H NMR (400 MHz, Methanol-d₄) δ 7.45-7.31 (m, 2H), 7.23-7.08 (m, 4H), 6.90-6.84 (m, 2H), 4.95-4.90 (m, 1H), 4.51-4.33 (m, 3H), 4.15 (s, 1H), 3.78 (d, J=2.8 Hz, 3H), 3.56 (d, J=11.9 Hz, 1H), 3.25-3.17 (m, 1H), 3.14-3.04 (m, 1H), 2.96 (d, J=7.2 Hz, 1H).

Prep-HPLC-conditions: Xselect CSH Prep C18 Column, 30*150 mm, 5 μm; Mobile Phase A: Water (0.05% TFA), Mobile Phase B: ACN; Flow rate: 60 mL/min; Gradient: 8% B to 38% B in 10 min; Wave Length: 254 nm/220 nm; RT1(min): 10.37.

### Synthetic Procedure of (2R,3S,4S)-4-hydroxy-2-{[4-(1,3-thiazol-5-yl)phenyl]methyl}pyrrolidin-3-yl N-[(3-fluorophenyl)methyl]carbamate (V190546)

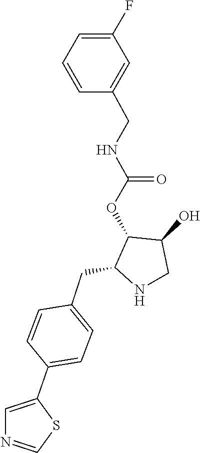 [SMILES: O=C(NCc1cccc(F)c1)O[C@@H]1[C@@H](O)CN[C@@H]1Cc1ccc(-c2cncs2)cc1]

The title compound was prepared in 29.6% overall yield as an off-white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-thiazol-5-yl)phenyl]methyl}pyrrolidine-1-carboxylate (Int-12) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z: Calc'd for C₂₂H₂₂FN₃O₃S [M+H]⁺428. found 428. ¹H NMR (400 MHz, Methanol-d₄) δ 8.99 (s, 1H), 8.17 (s, 1H), 7.67-7.60 (m, 2H), 7.43-7.34 (m, 3H), 7.17 (d, J=7.7 Hz, 1H), 7.11 (d, J=9.8 Hz, 1H), 7.04 (t, J=8.5 Hz, 1H), 4.97 (s, 1H), 4.42 (s, 1H), 4.36 (d, J=21.3 Hz, 2H), 4.29-4.23 (m, 1H), 3.60 (dd, J=12.5, 4.4 Hz, 1H), 3.26 (d, J=12.8 Hz, 1H), 3.20 (d, J=7.8 Hz, 1H), 3.10 (dd, J=14.1, 8.2 Hz, 1H).

Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 8% B to 38% B in 10 min; Wave Length: 254 nm/220 nm; RT1(min): 10.15.

### Synthetic Procedure of (2R,3S,4S)-4-hydroxy-2-{[4-(1,3-thiazol-4-yl)phenyl]methyl}pyrrolidin-3-yl N-[(3-fluorophenyl)methyl]carbamate (V190511)

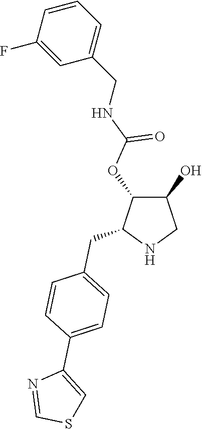 [SMILES: O=C(NCc1cccc(F)c1)O[C@@H]1[C@@H](O)CN[C@@H]1Cc1ccc(-c2cscn2)cc1]

The title compound was prepared in 24.2% overall yield as a white solid according to Stille Coupling with Pd(PPh₃)₄, CuI and CsF; General Procedure IV using 4-bromo-1,3-thiazole in STEP 1; Hydrolysis reaction with LiOH; General Procedure V in STEP 2; Addition reaction: Isocyanates and hydroxyl groups; General Procedure VI using 1-fluoro-3-(isocyanatomethyl)benzene in STEP 3; Boc Deprotection; General Procedure II in STEP 4. MS: m/z: Calc'd for C₂₂H₂₂FN₃O₃S [M+H]⁺428. found 428. ¹H NMR (400 MHz, Methanol-d₄) δ 9.08 (d, J=1.9 Hz, 1H), 7.94 (d, J=8.1 Hz, 2H), 7.89 (d, J=2.0 Hz, 1H), 7.39 (dd, J=7.1, 5.2 Hz, 3H), 7.17 (d, J=7.6 Hz, 1H), 7.10 (dd, J=10.0, 2.6 Hz, 1H), 7.07-6.99 (m, 1H), 5.03-4.98 (m, 1H), 4.46-4.22 (m, 4H), 3.61 (dd, J=12.7, 4.3 Hz, 1H), 3.29-3.18 (m, 2H), 3.09 (dd, J=14.0, 8.4 Hz, 1H).

Prep-HPLC purification conditions: Column: Xselect CSH Prep C18 Column, 30*150 mm, 5 m; Mobile Phase A: Water (0.05% TFA), Mobile Phase B: ACN; Flow rate: 60 mL/min; Gradient: 10% B to 40% B in 10 min; Wave Length: 254 nm/220 nm; RT1 (min): 10.72.

### Synthetic Procedure of (2R,3S,4S)-4-hydroxy-2-{[4-(imidazol-1-yl)phenyl]methyl}pyrrolidin-3-yl N-[(3-fluorophenyl)methyl]carbamate (V190515)

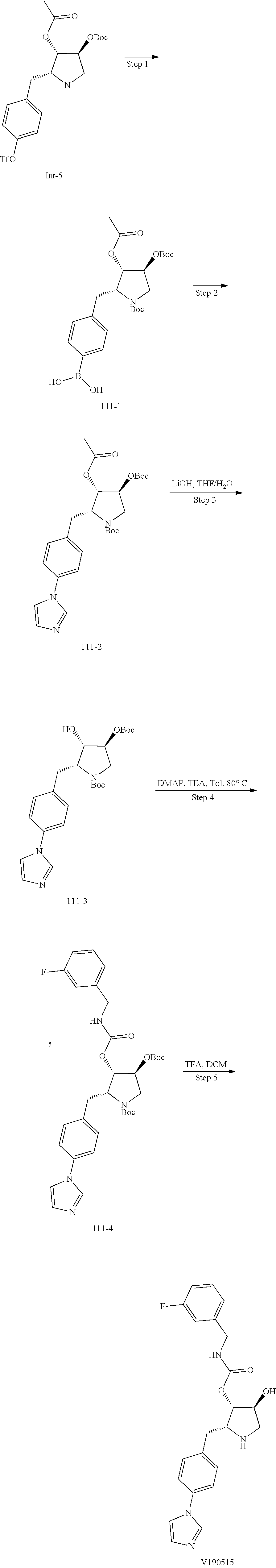

Step 1: To a stirred mixture of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (Int-5, 500 mg, 0.85 mmol, 1 equiv.), B₂(OH)₄ (307.5 mg, 3.48 mmol, 4 equiv.) and DIEA (664.4 mg, 5.12 mmol, 6 equiv.) in EtOH (5 mL) were added [3-(diphenylphosphanyl)propyl]diphenylphosphane; dichloronickel (92.8 mg, 0.1 mmol, 0.2 equiv.) and PPh₃ (89.8 mg, 0.34 mmol, 0.4 equiv.) at room temperature under nitrogen atmosphere. The resulting mixture was stirred for overnight at 80° C. under nitrogen atmosphere. The mixture was purified by reversed-phase flash chromatography to afford 4-{[(2R,3S,4S)-3-(acetyloxy)-1-(tert-butoxycarbonyl)-4-[(tert-butoxycarbonyl)oxy]pyrrolidin-2-yl]methyl}phenylboronic acid (120 mg, 29.2%) as a yellow oil.

Step 2: To a stirred mixture of 4-{[(2R,3S,4S)-3-(acetyloxy)-1-(tert-butoxycarbonyl)-4-[(tert-butoxycarbonyl)oxy]pyrrolidin-2-yl]methyl}phenylboronic acid (111-1, 80 mg, 0.17 mmol, 1 equiv.) and imidazole (22.7 mg, 0.34 mmol, 2 equiv.) in THF (3 mL) and H₂O (1 mL) was added Cu(OAc)₂ (15.16 mg, 0.08 mmol, 0.5 equiv.) at room temperature under nitrogen atmosphere. The resulting mixture was stirred for overnight at 50° C. under nitrogen atmosphere. The resulting mixture was filtered and the filter cake was washed with THF (1×2 mL). The filtrate was concentrated under reduced pressure. The residue was dissolved in MeCN (5 mL). The residue was purified by reversed-phase flash chromatography to afford tert-butyl (2R,3S,4S)-3-(acetyloxy)-4-[(tert-butoxycarbonyl)oxy]-2-{[4-(imidazol-1-yl)phenyl]methyl}pyrrolidine-1-carboxylate (111-2, 60 mg, 71.6%) as a yellow oil. MS: m/z: Calc'd for C₂₆H₃₅N₃O₇ [M+H]⁺ 502. found 502.

### Step 3, 4, and 5

The title compound was prepared in 22.2% overall yield as a white solid according to Hydrolysis reaction with LiOH; General Procedure V in STEP 3; Addition reaction: Isocyanates and hydroxyl groups; General Procedure VI using 1-fluoro-3-(isocyanatomethyl)benzene in STEP 4; Boc Deprotection; General Procedure II in STEP 5. MS: m/z: Calc'd for C₂₂H₂₃FN₄O₃ [M+H]⁺411. found 411. ¹H NMR (400 MHz, Methanol-d₄) δ 9.25 (s, 1H), 7.84 (d, J=113.5 Hz, 2H), 7.68-7.54 (m, 4H), 7.43-7.33 (m, 1H), 7.16 (d, J=7.7 Hz, 1H), 7.12-7.06 (m, 1H), 7.05-6.97 (m, 1H), 4.98-4.93 (m, 2H), 4.44-4.40 (m, 1H), 4.35 (d, J=13.7 Hz, 2H), 4.32-4.25 (m, 1H), 3.64 (dd, J=12.7, 4.3 Hz, 1H), 3.47-3.40 (m, 1H), 3.29-3.18 (m, 3H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 2% B to 23% B in 10 min; Wave Length: 254 nm/220 nm; RT1 (min): 9.82.

### Synthetic Procedure of (2R,3S,4S)-2-(1,3-benzothiazol-5-ylmethyl)-4-hydroxypyrrolidin-3-ylN-[(3-fluorophenyl)methyl]carbamate (V190552)

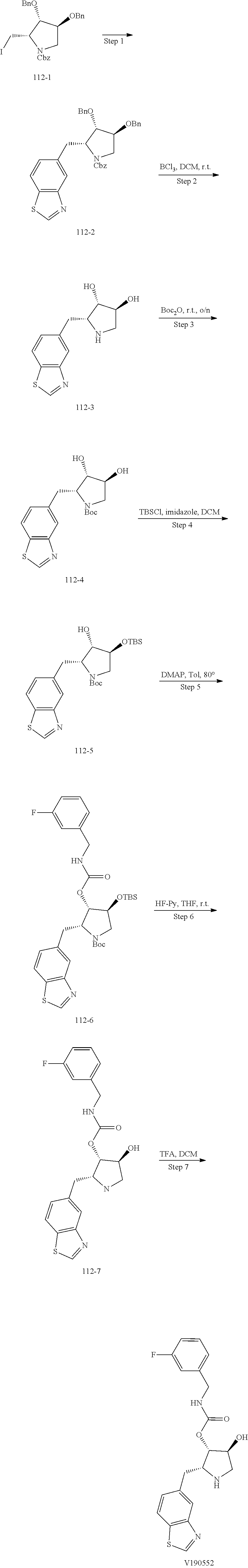

Step 1: To a stirred mixture of benzyl (2S,3S,4S)-3,4-bis(benzyloxy)-2-(iodomethyl)pyrrolidine-1-carboxylate (112-1, 1 g, 1.79 mmol, 1.00 equiv.) and Zinc (1.17 g, 17.94 mmol, 10 equiv.) in DMF (5 mL) was added iodine solution (2.28 g, 8.97 mmol, 5 equiv.) in DMF (2 mL) at room temperature under nitrogen atmosphere. The resulting mixture was stirred at room temperature for 1 h under nitrogen atmosphere. The resulting mixture was take the supernatant. To a stirred mixture of 5-bromo-1,3-benzothiazole (0.77 g, 3.58 mmol, 2 equiv.) and Pd₂(dba)₃ (0.33 g, 0.35 mmol, 0.2 equiv.), Q-Phos (0.26 g, 0.35 mmol, 0.2 equiv.) in THF (5 mL) was added supernatant at room temperature under nitrogen atmosphere. The resulting mixture was stirred at room temperature for overnight under nitrogen atmosphere. The resulting mixture was concentrated under reduced pressure. The residue was purified by reversed-phase flash chromatography to afford benzyl (2R,3S,4S)-2-(1,3-benzothiazol-5-ylmethyl)-3,4-bis(benzyloxy)pyrrolidine-1-carboxylate (400 mg, 39.4% yield, 85% purity) as a red oil. MS: m/z: Calc'd for C₃₄H₃₂N₂O₄S [M+H]⁺ 565. found 565.

Step 2: To a stirred mixture of benzyl (2R,3S,4S)-2-(1,3-benzothiazol-5-ylmethyl)-3,4-bis(benzyloxy)pyrrolidine-1-carboxylate (112-2, 450 mg, 0.79 mmol, 1 equiv) in DCM (10 mL) was added trichloroborane (8 mL) at room temperature under nitrogen atmosphere. The resulting mixture was stirred at room temperature for overnight under nitrogen atmosphere. The reaction was quenched with MeOH at 0° C. The resulting mixture was concentrated under reduced pressure. This resulted in (2R,3S,4S)-2-(1,3-benzothiazol-5-ylmethyl)pyrrolidine-3,4-diol (170 mg, 85.2% yield, 90% purity) as a yellow solid. MS: m/z: Calc'd for C₁₂H₁₄N₂O₂S [M+H]⁺ 251. found 251.

Step 3: To a stirred mixture of (2R,3S,4S)-2-(1,3-benzothiazol-5-ylmethyl)pyrrolidine-3,4-diol (200 mg, 0.79 mmol, 1 equiv) and Et₃N (242.5 mg, 2.39 mmol, 3 equiv.) in DCM (5 mL) was added di-tert-butyl dicarbonate (348.75 mg, 1.598 mmol, 2 equiv.) at room temperature. The resulting mixture was stirred at room temperature for overnight. The resulting mixture was concentrated under reduced pressure. The residue was purified by reversed-phase flash chromatography to afford tert-butyl (2R,3S,4S)-2-(1,3-benzothiazol-5-ylmethyl)-3,4-dihydroxypyrrolidine-1-carboxylate (200 mg, 71.4% yield, 85% purity) as a yellow solid. MS: m/z: Calc'd for C₁₇H₂₂N₂O₄S [M+22]⁺373. found 373.

Step 4: To a stirred mixture of tert-butyl (2R,3S,4S)-2-(1,3-benzothiazol-5-ylmethyl)-3,4-dihydroxypyrrolidine-1-carboxylate (112-3, 154 mg, 0.43 mmol, 1 equiv.) and Imidazole (89.7 mg, 1.31 mmol, 3 equiv.) in DCM (5 mL) was added tert-butyl(chloro)dimethylsilane (132.47 mg, 0.878 mmol, 2 equiv.) at 0° C. The resulting mixture was stirred at room temperature for 2 h and concentrated. The residue was purified by Prep-TLC (PE/EA 3:1) to afford tert-butyl (2R,3S,4S)-2-(1,3-benzothiazol-5-ylmethyl)-4-[(tert-butyldimethylsilyl)oxy]-3-hydroxypyrrolidine-1-carboxylate (100 mg, 48.9% yield, 90% purity) as a yellow oil. MS: m/z: Calc'd for C₂₃H₃₆N₂O₄SSi [M+22]⁺ 487, found 487.

### Step 5, 6, and 7

The title compound was prepared in 11.2% overall yield as a white solid according to Addition reaction: Isocyanates and hydroxyl groups; General Procedure VI using 1-fluoro-3-(isocyanatomethyl)benzene in STEP 5; TBS Deprotection; General Procedure VII in STEP 5; Boc Deprotection; General Procedure II in STEP 5. MS: m/z: Calc'd for C₂₀H₂₀FN₃O₃S [M+H]⁺402, found 402. ¹H NMR (400 MHz, Methanol-d₄) δ 9.30 (d, J=3.7 Hz, 1H), 8.11-8.02 (m, 2H), 7.47 (d, J=8.4 Hz, 1H), 7.43-7.33 (m, 1H), 7.17 (d, J=7.7 Hz, 1H), 7.09 (d, J=9.8 Hz, 1H), 7.03 (dd, J=9.8, 7.5 Hz, 1H), 5.02 (s, 1H), 4.44 (s, 1H), 4.45-4.30 (m, 3H), 3.66-3.57 (m, 1H), 3.39 (dd, J=14.2, 7.2 Hz, 1H), 3.30-3.13 (m, 2H).

Prep-HPLC purification conditions: Column: Xselect CSH Prep C18 Column, 30*250 mm, 10 m; Mobile Phase A: Water (0.05% TFA), Mobile Phase B: ACN; Flow rate: 60 mL/min; Gradient: 12% B to 42% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-{[4-(azetidin-3-yl)phenyl]methyl}carbamate (V190559)

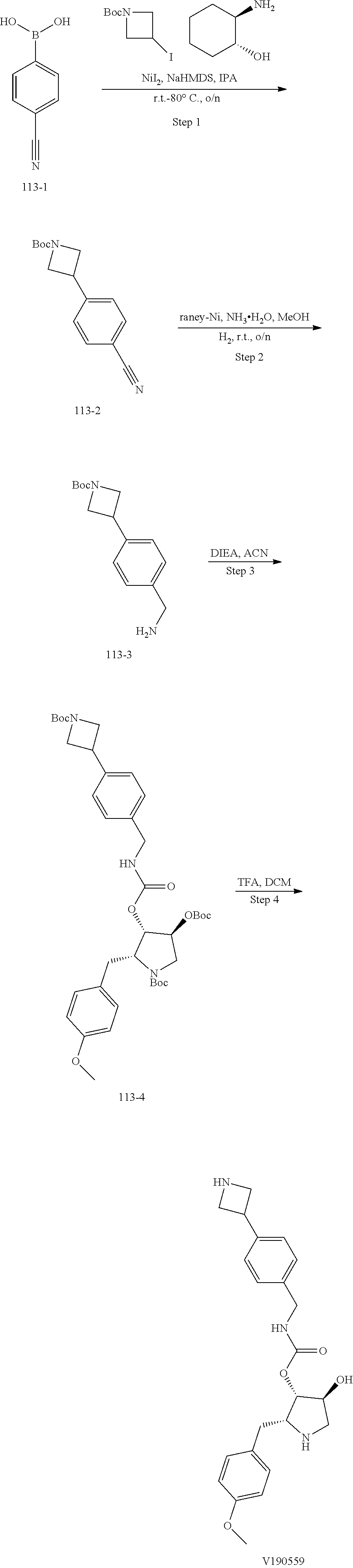

### Step 1

A solution of 4-cyanophenylboronic acid (113-1, 519.2 mg, 3.53 mmol, 2 equiv), nickel(II) iodide (55.9 mg, 0.17 mmol, 0.1 equiv.) and (1R,2R)-2-aminocyclohexan-1-ol (20.4 mg, 0.17 mmol, 0.1 equiv.) in IPA (10 mL) was treated with NaHMDS (1.8 mL, 3.52 mmol, 2 equiv.) and the resulting mixture was stirred for 1 h at room temperature under nitrogen atmosphere followed by the addition of tert-butyl 3-iodoazetidine-1-carboxylate (500 mg, 1.76 mmol, 1.00 equiv) at room temperature. The resulting mixture was stirred for overnight at 80° C. under nitrogen atmosphere. The resulting mixture was concentrated under reduced pressure. The residue was dissolved in DMSO (5 mL). The residue was purified by reversed-phase flash chromatography to afford tert-butyl 3-(4-cyanophenyl)azetidine-1-carboxylate (300 mg, 65.6%) as a white solid.

Step 2: To a stirred mixture of tert-butyl 3-(4-cyanophenyl)azetidine-1-carboxylate (113-2, 150 mg, 0.51 mmol, 1 equiv) and Raney-Ni (248.5 mg, 2.95 mmol, 5 equiv) in MeOH (3 mL) was added NH₃·H₂O (20.3 mg, 0.51 mmol, 1 equiv) at room temperature under nitrogen atmosphere. The resulting mixture was stirred overnight at room temperature under hydrogen atmosphere. The resulting mixture was filtered and the filter cake was washed with MeOH (2×2 mL). The filtrate was concentrated under reduced pressure. This resulted in tert-butyl 3-[4-(aminomethyl)phenyl]azetidine-1-carboxylate (100 mg, 65.6%) as a yellow oil.

### Step 3 and 4

The title compound was prepared in 49.5% overall yield as a white solid according to Substitution reaction for synthesis of Carbamate; General Procedure I using tert-butyl 3-[4-(aminomethyl)phenyl]azetidine-1-carboxylate (113-3) in STEP 3; Boc Deprotection; General Procedure II in STEP 4. MS: m/z: Calc'd for C₂₃H₂₉N₃O₄ [M+H]⁺412. found 412. ¹H NMR (400 MHz, Methanol-d₄) δ 7.40 (s, 2H), 7.24-7.15 (m, 1H), 6.91-6.83 (m, 1H), 4.93 (d, J=3.7 Hz, 1H), 4.43-4.33 (m, 2H), 4.33-4.10 (m, 3H), 3.79 (s, 2H), 3.59 (dd, J=12.6, 4.3 Hz, 1H), 3.37-3.30 (m, 1H), 3.22 (d, J=12.6 Hz, 1H).

Prep-HPLC purification conditions: Column: Xselect CSH Prep C18 Column, 30*150 mm, 5 m; Mobile Phase A: Water (0.05% TFA), Mobile Phase B: ACN; Flow rate: 60 mL/min; Gradient: 2% B to 21% B in 10 min; Wave Length: 254 nm/220 nm; RT1 (min): 9.83.

### Synthetic Procedure of (2R,3S,4S)-4-hydroxy-2-[(4-methoxyphenyl)methyl]pyrrolidin-3-yl N-{2-[(3S)-piperidin-3-yl]ethyl}carbamate (V191903)

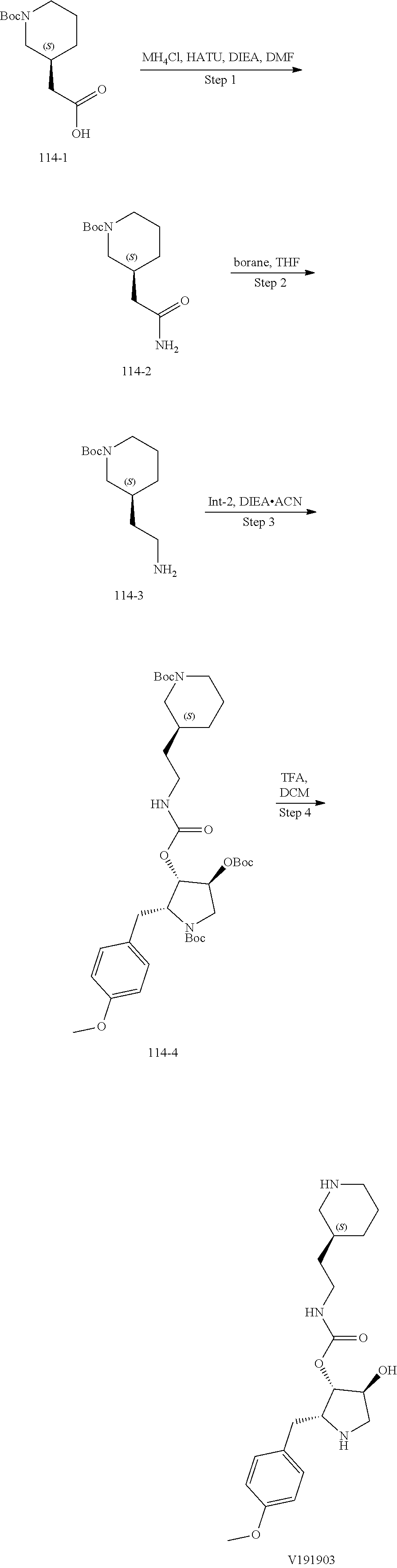

Step 1: To a stirred solution of [(3S)-1-(tert-butoxycarbonyl)piperidin-3-yl]acetic acid (114-1, 200 mg, 0.822 mmol, 1 equiv) and NH₄Cl (43.9 mg, 0.82 mmol, 1 equiv) in DMF (15 mL) were added HATU (625.1 mg, 1.64 mmol, 2 equiv) and DIEA (424.9 mg, 3.29 mmol, 4 equiv) at room temperature. The resulting mixture was stirred at room temperature for 6 h under nitrogen atmosphere. After completion of reaction monitored by LCMS. The resulting mixture was purified by reversed-phase flash chromatography to afford tert-butyl (3S)-3-(carbamoylmethyl)piperidine-1-carboxylate (200 mg, 99.4%) as a light yellow oil. MS: m/z: Calc'd for C₁₂H₂₂N₂O₃ [M+H]⁺243. found 243.

Step 2: To a stirred solution of tert-butyl (3S)-3-(carbamoylmethyl)piperidine-1-carboxylate (190 mg, 0.78 mmol, 1 equiv) in THF (10 mL) was added borane-THF (202.2 mg, 2.35 mmol, 3 equiv) at 0° C. under nitrogen atmosphere. The resulting mixture was stirred at room temperature for 2 h under nitrogen atmosphere. After completion of reaction monitored by LCMS. The crude product was used in the next step directly without further purification. MS: m/z: Calc'd for C₁₂H₂₄N₂O₂ [M+H]⁺229. found 229.

### Step 3 and 4

The title compound was prepared in 33.6% overall yield as a light yellow solid according to Substitution reaction for synthesis of Carbamate; General Procedure I using tert-butyl (3S)-3-(2-aminoethyl)piperidine-1-carboxylate (114-3) in STEP 3; Boc Deprotection; General Procedure II in STEP 4. MS: m/z: Calc'd for C₂₀H₃₁N₃O₄ [M+H]⁺527. found 527. ¹H NMR (400 MHz, Methanol-d₄) δ 7.27-7.20 (m, 2H), 6.97-6.89 (m, 2H), 4.96 (d, J=3.6 Hz, 1H), 4.38 (d, J=4.1 Hz, 1H), 4.22-4.13 (m, 1H), 3.80 (s, 3H), 3.57 (dd, J=12.7, 4.3 Hz, 1H), 3.38 (t, J=7.3 Hz, 2H), 3.30-3.17 (m, 3H), 3.12 (dd, J=14.2, 7.0 Hz, 1H), 3.02-2.87 (m, 2H), 2.70 (t, J=12.1 Hz, 1H), 2.04-1.92 (m, 2H), 1.88-1.66 (m, 2H), 1.64-1.46 (m, 2H), 1.37-1.22 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 24% B in 10 min; Wave Length: 254 nm/220 nm; RT1(min): 8.5.

### Synthetic Procedure of (2R,3S,4S)-4-hydroxy-2-[(4-methoxyphenyl)methyl]pyrrolidin-3-yl N-[(3,5-difluoro-4-hydroxyphenyl)methyl]carbamate (V191939)

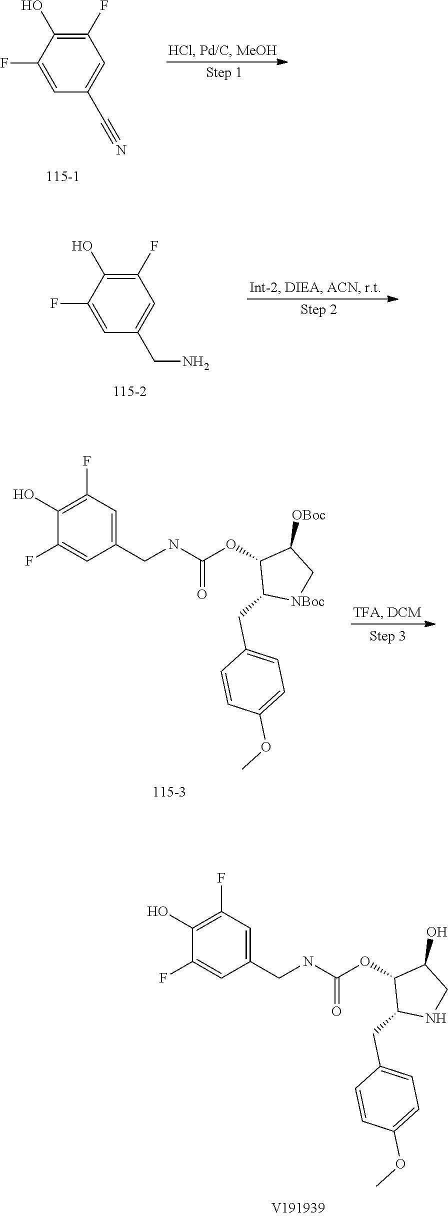

Step 1: To a solution of 3,5-difluoro-4-hydroxybenzonitrile (300 mg, 1.93 mmol, 1 equiv.) in methanol (8 mL) was added Pd/C (30 mg) and hydrochloric titrant (1 mL) at 0° C. under N₂. The suspension was degassed under vacuum and purged with H₂ several times. The mixture was stirred at under H₂ at r.t. for 1 h. TLC showed the reaction was completed. The reaction mixture was filtered and the filtrate was concentrated to give 4-(aminomethyl)-2,6-difluorophenol (150 mg, 48.7% yield, 73% purity) as a yellow solid. MS: m/z: Calc'd for C₇H₇F₂NO [M+H]⁺160. Found, 160.

Step 2 and 3: The title compound was prepared in 22.7% overall yield as a white solid according to Substitution reaction for synthesis of Carbamate; General Procedure I using 4-(aminomethyl)-2,6-difluorophenol (115-2) in STEP 2; Boc Deprotection; General Procedure II in STEP 3. MS: m/z: Calc'd for C₂₀H₂₂F₂N₂O₅ [M+H]⁺409. Found, 409. ¹H NMR (400 MHz, Methanol-d₄) δ 7.15-7.08 (m, 2H), 6.95-6.81 (m, 2H), 6.85-6.78 (m, 2H), 4.66-4.60 (m, 1H), 4.33-4.06 (m, 3H), 3.77 (s, 3H), 3.66-3.55 (m, 1H), 3.42-3.34 (m, 1H), 2.92-2.75 (m, 3H).

Prep-HPLC purification conditions: Column: XBridge Prep RP OBD C18 Column, 30*150 mm, 5 μm; Mobile Phase A: Water (10 mmol/L NH₄HCO₃), Mobile Phase B: ACN; Flow rate: 60 mL/min; Gradient: 3% B to 33% B in 10 min; Wave Length: 254 nm/220 nm; RT1(min): 9.4.

### Synthetic Procedure of (2R,3S,4S)-4-hydroxy-2-[(4-methoxyphenyl)methyl]pyrrolidin-3-yl N-[2-(1,1-dioxo-1lambda6,2-thiazolidin-2-yl)ethyl]carbamate (V191944)

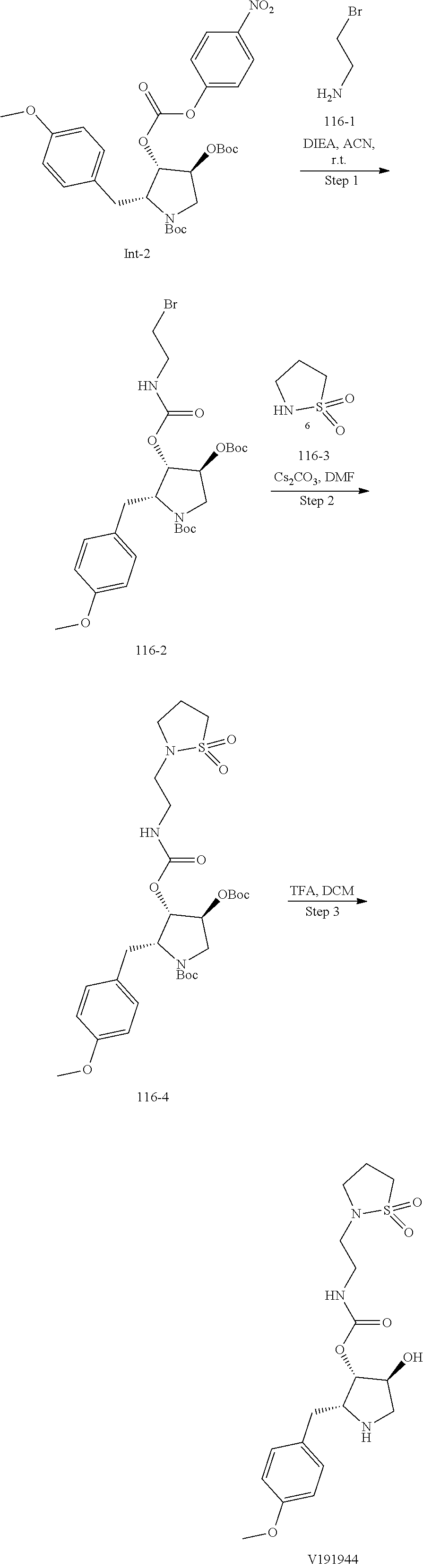

Step 1: The compound was prepared in 51.3% yield as a light yellow oil according to Substitution reaction for synthesis of Carbamate; General Procedure I using 2-bromoethanamine (116-1). MS: m/z: Calc'd for C₂₅H₃₇BrN₂O₈ [M+H−100−56]⁺417. Found, 417.

Step 2: To a stirred solution of tert-butyl (2R,3S,4S)-3-{[(2-bromoethyl)carbamoyl]oxy}-4-[(tert-butoxycarbonyl)oxy]-2-[(4-methoxyphenyl)methyl]pyrrolidine-1-carboxylate (116-2, 50 mg, 0.09 mmol, 1.0 equiv) and 1lambda6,2-thiazolidine-1,1-dione (116-3, 21.1 mg, 0.17 mmol, 2.0 equiv) in DMF (5 mL) was added Cs₂CO₃ (85.2 mg, 0.26 mmol, 3.0 equiv) in portions at room temperature.

The resulting mixture was stirred over night at 25° C. The solution was purified by reversed-phase flash chromatography to afford tert-butyl (2R,3S,4S)-4-[(tert-butoxycarbonyl)oxy]-3-({[2-(1,1-dioxo-1lambda6,2-thiazolidin-2-yl)ethyl]carbamoyl}oxy)-2-[(4-methoxyphenyl)methyl]pyrroli-dine-1-carboxylate (40 mg, 74.8%) as a light yellow oil. MS: m/z: Calc'd for C₂₈H₄₃N₃O₁₀S [M+H−100-56]⁺458. Found, 458.

Step 3: The title compound was prepared in 56.5% yield as a white solid according to Boc Deprotection; General Procedure II. MS: m/z: Calc'd for C₁₈H₂₇N₃O₆S [M+H]⁺414. Found, 414. ¹H NMR (400 MHz, Methanol-d4) δ 7.29-7.21 (m, 2H), 6.98-6.90 (m, 2H), 4.95-4.90 (m, 1H), 4.48-4.42 (m, 1H), 4.22-4.12 (m, 1H), 3.80 (s, 3H), 3.60 (dd, J=12.7, 4.3 Hz, 1H), 3.42-3.36 (m, 3H), 3.31-3.26 (m, 1H), 3.24-3.08 (m, 6H), 2.98 (dd, J=14.2, 8.5 Hz, 1H), 2.39-2.33 (m, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 30% B in 10 min; Wave Length: 254 nm/220 nm; RT1 (min): 9.5.

### Synthetic Procedure of (2R,3S,4S)-4-hydroxy-2-{[4-(trifluoromethyl)phenyl]methyl}pyrrolidin-3-yl N-[(3-fluorophenyl)methyl]carbamate (V192896)

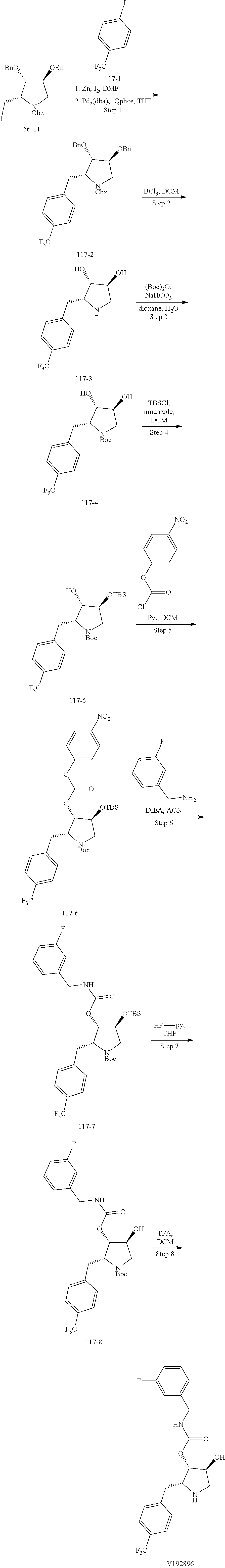

Step 1: A solution of benzyl (2S,3S,4S)-3,4-bis(benzyloxy)-2-(iodomethyl)pyrrolidine-1-carboxylate (1 g, 1.79 mmol, 1 equiv) in DMF (10 mL) was treated with zinc (1.17 g, 17.94 mmol, 10 equiv) and iodine (1.37 g, 5.38 mmol, 3 equiv) and the mixture was stirred for 1 h at 25° C. under nitrogen atmosphere. Then a mixture of 1-iodo-4-(trifluoromethyl)benzene (0.98 g, 3.59 mmol, 2 equiv) in THF (10 mL) was treated with Pd₂(dba)₃ (164.3 mg, 0.18 mmol, 0.1 equiv) and Q-Phos (255.0 mg, 0.36 mmol, 0.2 equiv) was added into the above mixture dropwise 2 min at 25° C. The resulting mixture was stirred for additional 12 h at 25° C. under N₂. The resulting mixture was purified by Prep-TLC (PE/EA, 5:1) to afford benzyl (2R,3S,4S)-3,4-bis(benzyloxy)-2-{[4-(trifluoromethyl)phenyl]methyl}pyrrolidine-1-carboxylate (400 mg, 39% yield, 70% purity) as a light yellow oil. MS: m/z: Calc'd for C₃₄H₃₂F₃NO₄ [M+H]⁺576. Found, 576.

Step 2: To a stirred solution of benzyl (2R,3S,4S)-3,4-bis(benzyloxy)-2-{[4-(trifluoromethyl)phenyl]methyl}pyrrolidine-1-carboxylate (395 mg, 0.69 mmol, 1 equiv) in DCM (1 mL) was added trichloroborane (1 M in DCM, 10.3 mL, 10.3 mmol, 15 equiv) dropwise at room temperature. The resulting mixture was stirred overnight at 25° C. The reaction was quenched with MeOH at 0° C. The resulting mixture was concentrated under reduced pressure. The crude product was used in the next step directly without further purification. MS: m/z: Calc'd for C₁₂H1₄F₃NO₂ [M+H]⁺ 262. Found, 262.

Step 3: To a stirred solution of (2R,3S,4S)-2-{[4-(trifluoromethyl)phenyl]methyl}pyrrolidine-3,4-diol (170 mg, 0.65 mmol, 1 equiv) in 1,4-dioxane (3 mL) and H₂O (1 mL) were added di-tert-butyl dicarbonate (426.0 mg, 1.95 mmol, 3 equiv) and NaHCO₃ (164.0 mg, 1.95 mmol, 3 equiv) in portions at 0° C. The resulting mixture was stirred 2 h at 25° C. and concentrated. The residue was purified by reversed-phase flash chromatography to afford tert-butyl (2R,3S,4S)-3,4-dihydroxy-2-{[4-(trifluoromethyl)phenyl]methyl}pyrrolidine-1-carboxylate (140 mg, 59.5% yield, 90% purity) as an off-white oil. MS: m/z: Calc'd for C₁₇H₂₂F₃NO₄ [M+H−56]⁺306. Found, 306.

Step 4: To a stirred solution of tert-butyl (2R,3S,4S)-3,4-dihydroxy-2-{[4-(trifluoromethyl)phenyl]methyl}pyrrolidine-1-carboxylate (140 mg, 0.39 mmol, 1 equiv) in DCM (5 mL) was added tert-butyl(chloro)dimethylsilane (175.18 mg, 1.16 mmol, 3 equiv) and imidazole (79.1 mg, 1.16 mmol, 3 equiv) in portions at 0° C. The resulting mixture was stirred 1 h at 25° C. The mixture was purified by Prep-TLC (PE/EA, 4:1) to afford tert-butyl (2R,3S,4S)-4-[(tert-butyldimethylsilyl)oxy]-3-hydroxy-2-{[4-(trifluoromethyl)phenyl]methyl}pyrrolidine-1-carboxylate (180 mg, 97.7% yield, 90% purity) as a light yellow oil. MS: m/z: Calc'd for C₂₃H₃₆F₃NO₄Si [M+H−100]⁺376. Found, 376.

Step 5: To a stirred solution of tert-butyl (2R,3S,4S)-4-[(tert-butyldimethylsilyl)oxy]-3-hydroxy-2-{[4-(trifluoromethyl)phenyl]methyl}pyrrolidine-1-carboxylate (170 mg, 0.36 mmol, 1 equiv) in DCM (5 mL) were added 4-nitrophenyl carbonochloridate (144.1 mg, 0.71 mmol, 2 equiv) and pyridine (56.6 mg, 0.71 mmol, 2 equiv) in portions at 0° C. The above mixture was stirred 3 h at 25° C. The resulting mixture was concentrated and the residue was purified by silica gel column chromatography, eluted with PE/EA (1:1) to afford tert-butyl (2R,3S,4S)-4-[(tert-butyldimethylsilyl)oxy]-3-[(4-nitrophenoxycarbonyl)oxy]-2-{[4-(trifluoromethyl)phenyl]-methyl}pyrrolidine-1-carboxylate (160 mg) as a light yellow oil. MS: m/z: Calc'd for C₃₀H₃₉F₃N₂O₈Si [M+H−100]⁺541. Found, 541.

### Step 6, 7, and 8

The title compound was prepared in 32.4% overall yield as a white solid according to Substitution reaction for synthesis of Carbamate; General Procedure I using tert-butyl (2R,3S,4S)-4-[(tert-butyldimethylsilyl)oxy]-3-[(4-nitrophenoxycarbonyl)oxy]-2-{[4-(trifluoromethyl)phenyl]methyl}pyrrolidine-1-carboxylate (117-6) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 6; TBS Deprotection; General Procedure VII in STEP 7; Boc Deprotection; General Procedure II in STEP 8. MS: m/z: Calc'd for C₂₀H₂₀F₄N₂O₃[M+H]⁺413. Found, 413. ¹H NMR (400 MHz, Methanol-d₄) δ 7.64 (d, J=7.8 Hz, 2H), 7.51 (d, J=8.0 Hz, 2H), 7.46-7.31 (m, 1H), 7.19-6.95 (m, 3H), 4.45-4.22 (m, 4H), 3.61 (dd, J=12.6, 4.3 Hz, 1H), 3.30-3.23 (m, 2H), 3.16 (dd, J=14.2, 8.2 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 20% B to 50% B in 10 min; Wave Length: 254 nm/220 nm; RT1 (min): 8.5.

### Synthetic Procedure of (2R,3S,4S)-2-(4-(fluoromethoxy)benzyl)-4-hydroxypyrrolidin-3-yl (3-fluorobenzyl)carbamate (V190507)

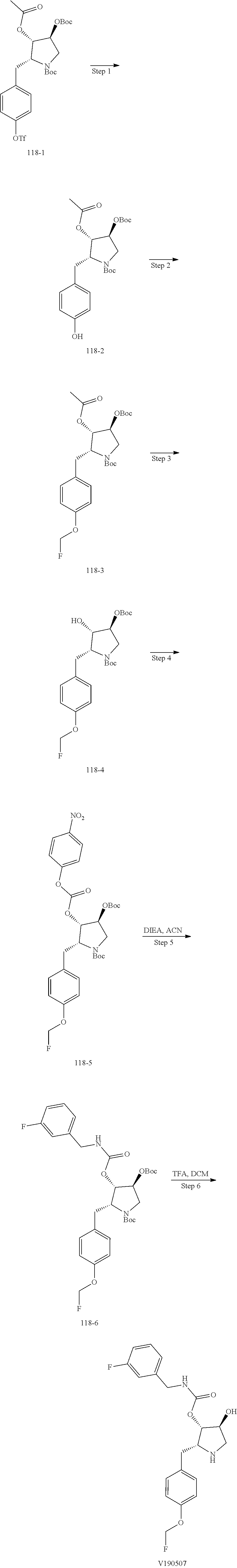

Step 1: To the solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (118-1, 500 mg, 0.86 mmol, 1 equiv) in DMF (5 mL) was added Tetramethylammonium fluoride (319 mg, 3.43 mmol, 4 equiv). The mixture was stirred at 80° C. for 5 hours under N₂. The mixture was purified by reversed-phase flash chromatography with the following conditions: column, C18 silica gel; mobile phase, MeCN in Water (0.1% TFA), 0% to 100% gradient in 150 min; detector, UV 220 nm. The tert-butyl (2R,3S,4S)-3-(acetyloxy)-4-[(tert-butoxycarbonyl)oxy]-2-[(4-hydroxyphenyl)methyl]pyrrolidine-1-carboxylate (250 mg, 64.6% yield) was obtained as a white solid. MS: m/z: Calc'd for C23H33NO8 [M+H]+452. found 452.

Step 2: To a stirred mixture of fluoroiodomethane (250 mg, 1.56 mmol, 1 equiv) and tert-butyl (2R,3S,4S)-3-(acetyloxy)-4-[(tert-butoxycarbonyl)oxy]-2-[(4-hydroxyphenyl)methyl]pyrrolidine-1-carboxylate (118-2, 1411.6 mg, 3.12 mmol, 2 equiv) in DMF (10 mL) was added K₂CO₃ (648.1 mg, 4.68 mmol, 3 equiv) at room temperature. The resulting mixture was stirred at 60° C. for overnight under nitrogen atmosphere. The residue was purified by reversed-phase flash chromatography with the following conditions: column, C18 silica gel; mobile phase, MeCN in Water (0.1% TFA), 0% to 100% gradient in 10 min; detector, UV 220 nm. This resulted in tert-butyl (2R,3S,4S)-3-(acetyloxy)-4-[(tert-butoxycarbonyl)oxy]-2-{[4-(fluoromethoxy)phenyl]methyl}pyrrolidine-1-carboxylate (200 mg) as a yellow oil.

MS: m/z: Calc'd for C₂₄H₃₄FNO₈ [M+H]+484. found 484.

Step 3: To a stirred mixture of tert-butyl (2R,3S,4S)-3-(acetyloxy)-4-[(tert-butoxycarbonyl)oxy]-2-{[4-(fluoromethoxy)phenyl]methyl}pyrrolidine-1-carboxylate (118-3, 200 mg, 0.41 mmol, 1 equiv) in THF (5 mL) and H₂O (1 mL) was added LiOH (29.7 mg, 1.24 mmol, 3. equiv) at room temperature. The resulting mixture was stirred at room temperature for overnight. The residue was purified by reversed-phase flash chromatography with the following conditions: column, C18 silica gel; mobile phase, MeCN in Water (0.1% TFA), 0% to 100% gradient in 10 min; detector, UV 220 nm. This resulted in tert-butyl (2R,3S,4S)-4-[(tert-butoxycarbonyl)oxy]-2-{[4-(fluoromethoxy)phenyl]methyl}-3-hydroxypyrrolidine-1-carboxylate (180 mg, 98.5% yield, 80% purity) as a yellow oil. MS: m/z: Calc'd for C₂₂H₃₂FNO₇ [M+H]+442. found 442.

Step 4: To a stirred mixture of tert-butyl (2R,3S,4S)-4-[(tert-butoxycarbonyl)oxy]-2-{[4-(fluoromethoxy)phenyl]methyl}-3-hydroxypyrrolidine-1-carboxylate (118-4, 180 mg, 0.40 mmol, 1 equiv) and pyridine (96.7 mL, 1.22 mmol, 3 equiv) in DCM (10 mL) was added 4-nitrophenyl chloroformate (164.3 mg, 0.81 mmol, 2 equiv) at room temperature. The resulting mixture was stirred at room temperature for overnight. The resulting mixture was concentrated under vacuum. The residue was dissolved in DCM (10 mL) 1:1) to afford tert-butyl (2R,3S,4S)-4-[(tert-butoxycarbonyl)oxy]-2-{[4-(fluoromethoxy)phenyl]methyl}-3-[(4-nitrophenoxycarbonyl)oxy]pyrrolidine-1-carboxylate (170 mg, 68.7% yield, 80% purity) as a white solid. MS: m/z: Calc'd for C₂₉H₃₅FN₂O₁₁ [M+H]⁺607. found 607.

### Step 5 and 6

The title compound was prepared in 20.6% overall yield as a white solid according to Substitution reaction for synthesis of Carbamate; General Procedure I using tert-butyl (2R,3S,4S)-4-((tert-butoxycarbonyl)oxy)-2-(4-(fluoromethoxy)benzyl)-3-(((4-nitrophenoxy)carbonyl)oxy)pyrrolidine-1-carboxylate (118-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (3-fluorophenyl)methanamine in STEP 5; Boc Deprotection; General Procedure II in STEP 6. MS: m/z: Calc'd for C₂₀H₂₂F₂N₂O₄[M+H]⁺393. found 393. ¹H NMR (400 MHz, Methanol-d4) δ 7.43-7.33 (m, 1H), 7.32-7.14 (m, 3H), 7.13-6.98 (m, 4H), 5.81 (s, 1H), 5.68 (s, 1H), 4.96 (s, 1H), 4.44-4.27 (m, 3H), 4.25-4.15 (m, 1H), 3.58 (dd, J=12.7, 4.3 Hz, 1H), 3.24 (d, J=12.6 Hz, 1H), 3.15 (dd, J=14.2, 7.4 Hz, 1H), 3.01 (dd, J=14.1, 8.3 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min mL/min; Gradient: 14% B to 44% B in 10 min; Wave Length: 254 nm/220 nm; RT1(min): 8.5

### Synthetic Procedure of (2R,3S,4S)-4-hydroxy-2-(4-(trifluoromethoxy)benzyl)pyrrolidin-3-yl (3-fluorobenzyl)carbamate (V190509)

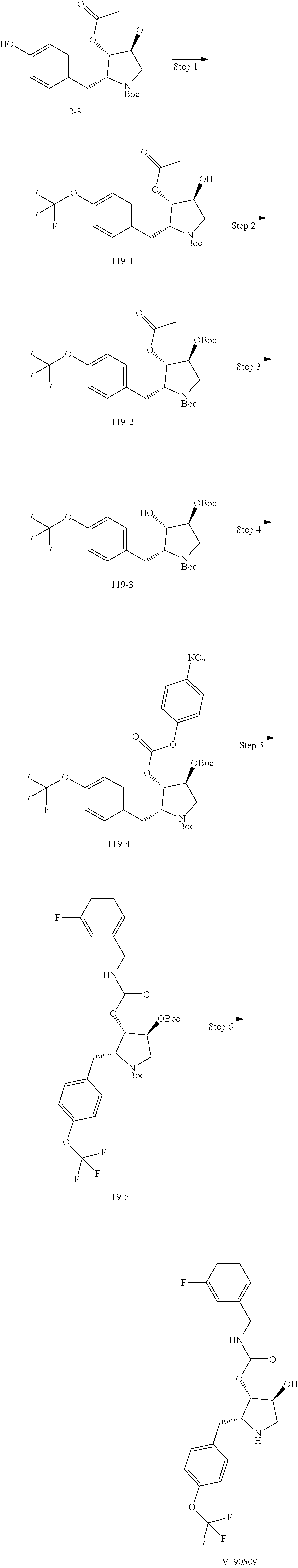

Step 1: To a stirred mixture of tert-butyl (2R,3S,4S)-3-(acetyloxy)-4-hydroxy-2-[(4-hydroxyphenyl)methyl]pyrrolidine-1-carboxylate (2-3, 100 mg, 0.28 mmol, 1 equiv) trimethyl(trifluoromethyl)silane (202.3 mg, 1.42 mmol, 5 equiv) Silver trifluoromethanesulfonate (365.5 mg, 1.45 mmol, 5 equiv) 6-fluoropyridine-3-carbonitrile (173.7 mg, 1.42 mmol, 5 equiv) 4-(chloromethyl)-1-fluoro-1,4-diazabicyclo[2.2.2]octane-1,4-diium (201.6 mg, 0.57 mmol, 2 equiv) and N-(benzenesulfonyl)-S-phenylfluoranesulfonamido (179.48 mg, 0.57 mmol, 2 equiv) in Toluene (6 mL) and (trifluoromethyl)benzene (3 mL) was added N-(benzenesulfonyl)-S-phenylfluoranesulfonamido (179.4 mg, 0.57 mmol, 2 equiv) and CsF (259.3 mg, 1.71 mmol, 6 equiv) at room temperature under nitrogen atmosphere. The resulting mixture was stirred at room temperature for overnight. The resulting mixture was filtered, the filter cake was washed with Toluene (6 mL). The filtrate was concentrated under reduced pressure. The residue was purified by reversed-phase flash chromatography with the following conditions: column, C18 silica gel; mobile phase, MeCN in Water (0.1% TFA), 10% to 50% gradient in 10 min; detector, UV 200 nm. This resulted in tert-butyl (2R,3S,4S)-3-(acetyloxy)-4-hydroxy-2-{[4-(trifluoromethoxy)phenyl]methyl}pyrrolidine-1-carboxylate (80 mg) as a white solid. MS: m/z: Calc'd for C₁₉H₂₄F₃NO₆ [M+H]+ 420. found 420.

Step 2: To a stirred mixture of tert-butyl (2R,3S,4S)-3-(acetyloxy)-4-hydroxy-2-{[4-(trifluoromethoxy)phenyl]methyl}pyrrolidine-1-carboxylate (119-1, 84 mg, 0.20 mmol, 1 equiv) and Boc₂O (109.2 mg, 0.50 mmol, 5 equiv) in Pyridine (5 mL) was added DMAP (18.3 mg, 0.15 mmol, 1.5 equiv) at room temperature. The resulting mixture was stirred at room temperature for overnight. The resulting mixture was concentrated under reduced pressure. The residue was dissolved in MeCN (5 mL). This resulted in tert-butyl (2R,3S,4S)-3-(acetyloxy)-4-[(tert-butoxycarbonyl)oxy]-2-{[4-(trifluoromethoxy)phenyl]methyl}pyrrolidine-1-carboxylate (80 mg, 76.8%) as a yellow solid. MS: m/z: Calc'd for C₂₄H₃₂F₃NO₈ [M+H]+ 520. found 520.

Step 3: To a stirred mixture of tert-butyl (2R,3S,4S)-3-(acetyloxy)-4-[(tert-butoxycarbonyl)oxy]-2-{[4-(trifluoromethoxy)phenyl]methyl}pyrrolidine-1-carboxylate (119-2, 67 mg, 0.12 mmol, 1 equiv) in THF (5 mL) and H₂O (0.5 mL) was added LiOH (9.2 mg, 0.38 mmol, 3 equiv) at room temperature. The resulting mixture was stirred for overnight at room temperature. The reaction mixture was quenched with water and extracted with EA. The extracts were washed with brine, dried over anhydrous sodium sulfate, filtered and concentrated. This resulted in tert-butyl (2R,3S,4S)-4-[(tert-butoxycarbonyl)oxy]-3-hydroxy-2-{[4-(trifluoromethoxy)phenyl]methyl}pyrrolidine-1-carboxylate (60 mg, 88.2%) as a yellow oil. MS: m/z: Calc'd for C₂₂H₃₀F₃NO₇ [M+H]+ 478. found 478.

Step 4: To a stirred mixture of tert-butyl (2R,3S,4S)-4-[(tert-butoxycarbonyl)oxy]-3-hydroxy-2-{[4-(trifluoromethoxy)phenyl]methyl}pyrrolidine-1-carboxylate (119-3, 60 mg, 0.12 mmol, 1 equiv) and Pyridine (29.8 mg, 0.37 mmol, 3 equiv) in DCM (5 mL) was added 4-nitrophenyl chloroformate (37.9 mg, 0.18 mmol, 1.5 equiv) at room temperature. The resulting mixture was stirred at room temperature for overnight. The residue was purified by Prep-TLC (PE/EA 1:1) to afford tert-butyl (2R,3S,4S)-4-[(tert-butoxycarbonyl)oxy]-3-[(4-nitrophenoxycarbonyl)oxy]-2-{[4-(trifluoromethoxy)phenyl]methyl}pyrrolidine-1-carboxylate (80 mg) as a yellow oil. MS: m/z: Calc'd for C₂₉H₃₃F₃N₂O₁₁ [M+H]+ 643. found 643.

### Step 5 and 6

The title compound was prepared in 26.5% overall yield as a white solid according to Substitution reaction for synthesis of Carbamate; General Procedure I using tert-butyl (2R,3S,4S)-4-((tert-butoxycarbonyl)oxy)-3-(((4-nitrophenoxy)carbonyl)oxy)-2-(4-(trifluoromethoxy)benzyl)pyrrolidine-1-carboxylate (119-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (3-fluorophenyl)methanamine in STEP 5; Boc Deprotection; General Procedure II in STEP 6. MS: m/z: Calc'd for C₂₀H₂₀F₄N₂O₄[M+H]+ 429. found 429.

¹H NMR (400 MHz, Methanol-d4) δ 7.38 (dd, J=32.2, 0.0 Hz, 3H), 7.25 (d, J=7.7 Hz, 2H), 7.15 (d, J=7.7 Hz, 1H), 7.12-7.05 (m, 1H), 7.08-6.98 (m, 1H), 4.98 (d, J=3.5 Hz, 1H), 4.44-4.39 (m, 1H), 4.35 (d, J=14.9 Hz, 2H), 4.30-4.20 (m, 1H), 3.59 (dd, J=12.6, 4.3 Hz, 1H), 3.28-3.17 (m, 2H), 3.08 (dd, J=14.1, 8.4 Hz, 1H).

Prep-HPLC purification conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 18% B to 48% B in 10 min; Wave Length: 254 nm/220 nm; RT1(min): 8.35.

### Synthetic Procedure of (2R,3S,4S)-4-methoxy-2-(4-(oxazol-5-yl)benzyl)pyrrolidin-3-yl (3-fluorobenzyl)carbamate (V192976)

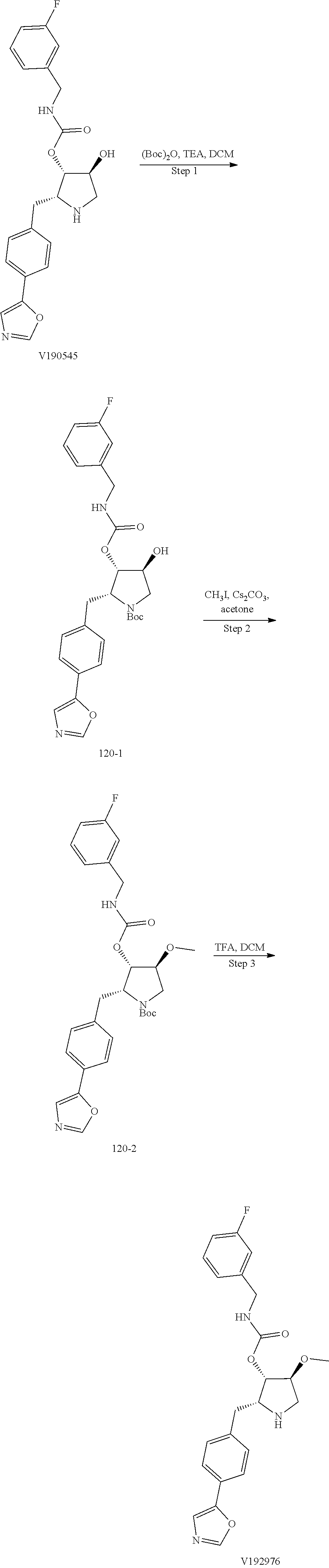 [SMILES: O=C(NCc1cccc(F)c1)O[C@@H]1[C@@H](O)CN[C@@H]1Cc1ccc(-c2cnco2)cc1 ; CC(C)(C)OC(=O)N1C[C@H](O)[C@@H](OC(=O)NCc2cccc(F)c2)[C@H]1Cc1ccc(-c2cnco2)cc1 ; CO[C@H]1CN(C(=O)OC(C)(C)C)[C@H](Cc2ccc(-c3cnco3)cc2)[C@@H]1OC(=O)NCc1cccc(F)c1 ; CO[C@H]1CN[C@H](Cc2ccc(-c3cnco3)cc2)[C@@H]1OC(=O)NCc1cccc(F)c1]

Step 1: To a stirred solution of (2R,3S,4S)-4-hydroxy-2-{[4-(1,3-oxazol-5-yl)phenyl]methyl}pyrrolidin-3-yl N-[(3-fluorophenyl)methyl]carbamate (V190545, 80 mg, 0.194 mmol, 1 equiv) in DCM (3 mL) was added di-tert-butyl dicarbonate (127.3 mg, 0.58 mmol, 3 equiv) and Et3N (59.0 mg, 0.58 mmol, 3 equiv) at 0° C. The resulting mixture was stirred 1 h at 25° C. The residue was purified by silica gel column chromatography, eluted with PE/EA (4:1) to afford tert-butyl (2R,3S,4S)-3-({[(3-fluorophenyl)methyl]carbamoyl}oxy)-4-hydroxy-2-{[4-(1,3-oxazol-5-yl)phenyl]methyl}pyrrolidine-1-carboxylate (60 mg) as a light yellow solid. MS: m/z: Calc'd for C₂₇H₃₀FN₃O₆ [M+H]⁺512. Found, 512.

Step 2: To a stirred solution of tert-butyl (2R,3S,4S)-3-({[(3-fluorophenyl)methyl]carbamoyl}oxy)-4-hydroxy-2-{[4-(1,3-oxazol-5-yl)phenyl]methyl}pyrrolidine-1-carboxylate (120-1, 90 mg, 0.18 mmol, 1 equiv) in acetone (14 mL) was added CH₃I (124.9 mg, 0.9 mmol, 5 equiv) and Cs₂CO₃ (172 mg, 0.53 mmol, 3 equiv) at room temperature. The resulting mixture was stirred overnight at 25° C. The resulting mixture was filtered, the filter cake was washed with EtOAc (3×1 mL). The filtrate was concentrated under reduced pressure. The residue was purified by silica gel column chromatography, eluted with PE/EA (1:1) to afford tert-butyl (2R,3S,4S)-3-({[(3-fluorophenyl)methyl]carbamoyl}oxy)-4-methoxy-2-{[4-(1,3-oxazol-5-yl)phenyl]methyl}pyrrolidine-1-carboxylate (40 mg) as a light yellow oil. MS: m/z: Calc'd for C₂₈H₃₂FN₃O₆ [M+H]⁺526. Found, 526.

Step 3: The title compound was prepared in 80.9% overall yield as a white solid according to Boc Deprotection; General Procedure II in STEP 3. MS: m/z: Calc'd for C₂₃H₂₄FN₃O₄[M+H]⁺426. Found, 426.

¹H NMR (400 MHz, Methanol-d₄) δ 8.28 (d, J=3.1 Hz, 1H), 7.75 (d, J=8.1 Hz, 2H), 7.55 (s, 1H), 7.37 (dd, J=35.0, 8.0 Hz, 3H), 7.18-7.03 (m, 3H), 5.09 (d, J=15.6 Hz, 1H), 4.65 (d, J=16.3 Hz, 1H), 4.46-4.39 (m, 1H), 4.36-4.26 (m, 1H), 3.68-3.40 (m, 1H), 3.31-3.19 (m, 2H), 3.12 (d, J=8.9 Hz, 1H), 3.05 (d, J=5.9 Hz, 4H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min mL/min; Gradient: 14% B to 44% B in 10 min; Wave Length: 254 nm/220 nm; RT1(min): 8.5.

### Synthetic Procedure of (2R,3S,4S)-4-hydroxy-1-methyl-2-(4-(oxazol-5-yl)benzyl)pyrrolidin-3-yl (3-fluorobenzyl)carbamate (V192977)

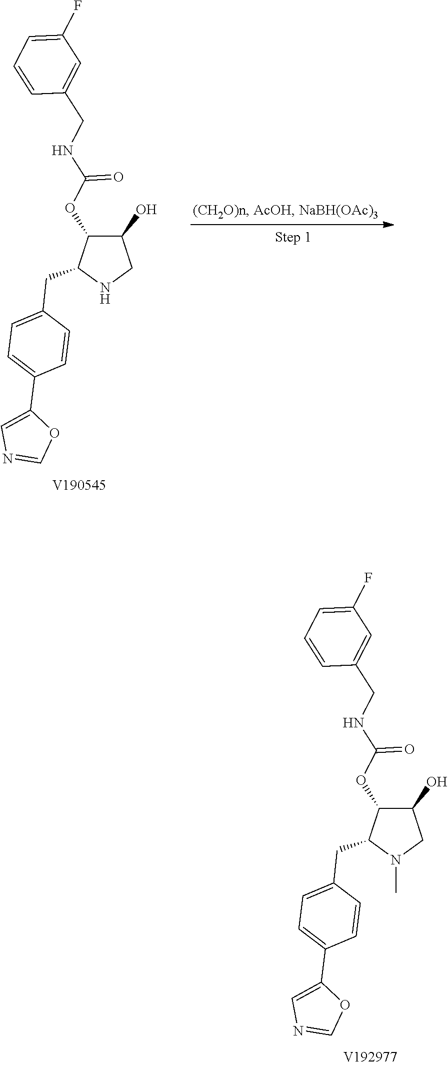 [SMILES: O=C(NCc1cccc(F)c1)O[C@@H]1[C@@H](O)CN[C@@H]1Cc1ccc(-c2cnco2)cc1 ; CN1C[C@H](O)[C@@H](OC(=O)NCc2cccc(F)c2)[C@H]1Cc1ccc(-c2cnco2)cc1]

Step 1: To a stirred solution of (2R,3S,4S)-4-hydroxy-2-{[4-(1,3-oxazol-5-yl)phenyl]methyl}pyrrolidin-3-yl N-[(3-fluorophenyl)methyl]carbamate (V190545, 50 mg, 0.12 mmol, 1 equiv) and (HCHO)n (10.9 mg, 0.37 mmol, 3 equiv) in DCM (3 mL) was added HOAc (14.6 mg, 0.24 mmol, 2 equiv) at 0° C. The resulting mixture was stirred 0.5 h at 0° C. Then add STAB (51.5 mg, 0.24 mmol, 2 equiv) at 0° C. The resulting mixture was stirred 2 h at 25° C. The resulting mixture was filtered, the filter cake was washed with DCM (3 mL)(1×1 mL). The filtrate was concentrated under reduced pressure. The mixture was purified directly by reversed-phase column chromatography (10 mmol/L NH₄HCO₃ in water and acetonitrile) to afford (2R,3S,4S)-4-hydroxy-1-methyl-2-{[4-(1,3-oxazol-5-yl)phenyl]methyl}pyrrolidin-3-yl N-[(3-fluorophenyl)methyl]carbamate; trifluoroacetic acid (6.2 mg) as a white solid. MS: m/z: Calc'd for C₂₃H₂₄FN₃O₄[M+H]⁺426. Found, 426. ¹H NMR (400 MHz, Methanol-d₄) δ 8.29 (s, 1H), 7.68 (d, J=8.1 Hz, 2H), 7.52 (s, 1H), 7.39 (dd, J=8.1, 5.1 Hz, 3H), 7.17 (d, J=7.6 Hz, 1H), 7.14-7.03 (m, 2H), 4.44-4.34 (m, 2H), 4.29 (d, J=15.4 Hz, 1H), 4.22-4.14 (m, 1H), 4.05 (dd, J=13.0, 4.5 Hz, 1H), 3.38 (s, 1H), 3.28-3.21 (m, 1H), 3.10 (s, 3H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min mL/min; Gradient: 12% B to 42% B in 15 min; Wave Length: 254 nm/220 nm; RT1(min): 14.5.

### Synthetic Procedure of (2R,3S,4S)-4-hydroxy-2-(4-(oxazol-5-yl)benzyl)pyrrolidin-3-yl (2-((R)-azepan-3-yl)ethyl)carbamate (V193012)

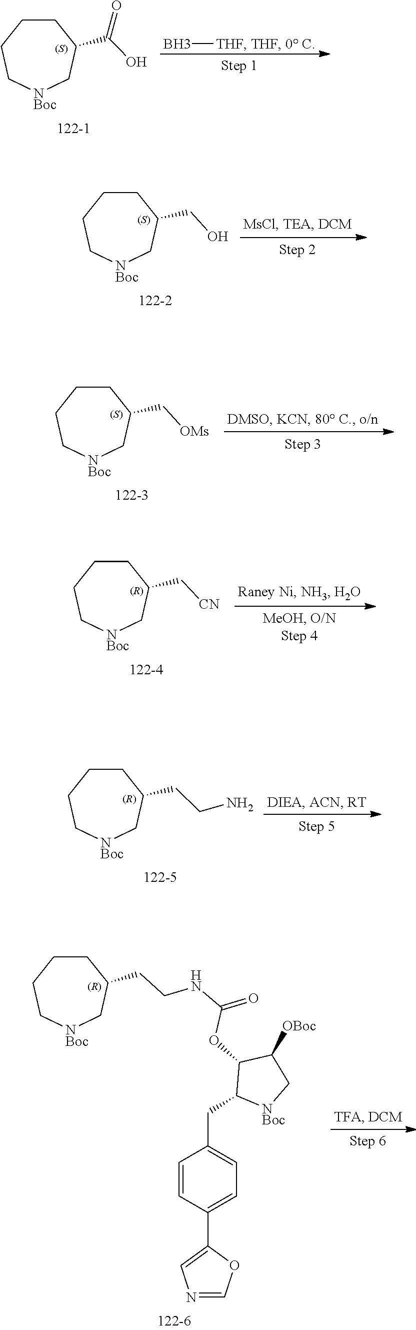

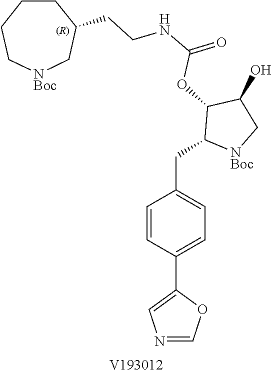

Step 1: To a stirred solution of (3S)-1-(tert-butoxycarbonyl)azepane-3-carboxylic acid (122-1, 500 mg, 2.05 mmol, 1 equiv) in anhydrous THF (10 mL) was added Borane-tetrahydrofuran complex (1.0M in THF) (4.1 mL, 4.1 mmol, 2 equiv) at 0° C. and stirred at 60° C. for 2h. Upon completed, the reaction mixture was concentrated under reduced pressure to give crude product which was further purified by silica gel column chromatography, eluted with PE/EA (1:1) to afford tert-butyl (3S)-3-(hydroxymethyl)azepane-1-carboxylate (500 mg, 106.10% yield, 90% purity) as a colorless oil. C₁₂H₂₃NO₃ [M−56]⁺ 174. found 174.

Step 2: To a stirred solution of tert-butyl (3S)-3-(hydroxymethyl)azepane-1-carboxylate (122-2, 500 mg, 2.18 mmol, 1 equiv) in anhydrous DCM (10 mL) was added methanesulfonyl chloride (499.5 mg, 4.36 mmol, 2 equiv) and Et3N (6.5 mL, 6.54 mmol, 3 equiv) at 0° C. and stirred at room temperature for overnight. Upon completed, the reaction mixture was concentrated under reduced pressure to give crude product which was further purified by silica gel column chromatography, eluted with PE/EA (2:1) to afford tert-butyl (3S)-3-[(methanesulfonyloxy)methyl]azepane-1-carboxylate (600 mg, 89.52% yield, 97% purity) as a colorless oil. MS: m/z: Calc'd for C₁₃H₂₅NO₅S [M−56]⁺ 252, found 252.

Step 3: To a stirred solution of tert-butyl (3S)-3-[(methanesulfonyloxy)methyl]azepane-1-carboxylate (122-3, 600 mg, 1.95 mmol, 1 equiv) in anhydrous DMSO (10 mL) was added KCN (381.3 mg, 5.86 mmol, 3 equiv) at room temperature and stirred at 80° C. for overnight. Upon completed, the resulting mixture was extracted with EtOAc (2×100 mL). The combined organic layers were washed with brine (1×100 mL), dried over anhydrous Na₂SO₄. After filtration, the filtrate was concentrated under reduced pressure to give crude product which was further purified by silica gel column chromatography, eluted with PE/EA (2:1) to afford tert-butyl (3R)-3-(cyanomethyl)azepane-1-carboxylate (400 mg, 85.99% yield, 100% purity) as a colorless oil. MS: m/z: Calc'd for C₁₃H₂₂N₂O₂ [M−56]⁺ 183. found 183.

Step 4: To a stirred solution of tert-butyl (3R)-3-(cyanomethyl)azepane-1-carboxylate (122-4, 95 mg, 0.4 mmol, 1 equiv) in anhydrous methanol (3 mL) and NH₃·H₂O (0.3 mL) was added raney nickel (70.2 mg, 1.19 mmol, 3 equiv) and hydrogenated at room temperature under 20 psi of hydrogen pressure for overnight, filtered through a Celite pad and concentrated under reduced pressure to give crude product tert-butyl (3R)-3-(2-aminoethyl)azepane-1-carboxylate (80 mg) as a colorless oil. MS: m/z: Calc'd for C₁₃H₂₆N₂O₂ [M+H]⁺ 243. found 243.

### Step 5 and 6

The title compound was prepared in 43.5% overall yield as a light yellow solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (R)-3-(2-aminoethyl)azepane-1-carboxylate (122-5) in STEP 1; Boc Deprotection; General Procedure II in STEP 2. MS: m/z: Calc'd for C₂₃H₃₂N₄O₄ [M+H]⁺429. found 429. ¹H NMR (400 MHz, Methanol-d4) δ 8.27 (s, 1H), 7.79-7.72 (m, 2H), 7.54 (s, 1H), 7.45 (d, J=8.2 Hz, 2H), 5.03-4.97 (m, 1H), 4.43-4.37 (m, 1H), 4.32-4.21 (m, 1H), 3.61 (dd, J=12.2, 4.0 Hz, 1H), 3.35 (d, J=3.5 Hz, 1H), 3.23 (dt, J=11.1, 7.1 Hz, 6H), 3.11 (dd, J=14.3, 8.6 Hz, 1H), 2.96 (dd, J=13.5, 10.1 Hz, 1H), 2.17-1.76 (m, 5H), 1.68-1.54 (m, 3H), 1.48-1.35 (m, 1H).

Prep-HPLC conditions: Column: XBridge Prep OBD C18 Column, 30*150 mm, 5 μm; Mobile Phase A: Water (0.05% TFA), Mobile Phase B: ACN; Flow rate: 60 mL/min mL/min; Gradient: 2% B to 25% B in 10 min; Wave Length: 254 nm/220 nm; RT1(min): 8.5.

### Synthetic Procedure of (2R,3S,4S)-4-hydroxy-2-(4-(oxazol-5-yl)benzyl)pyrrolidin-3-yl (2-((R)-5-azaspiro[2.4]heptan-6-yl)ethyl)carbamate (V193018)

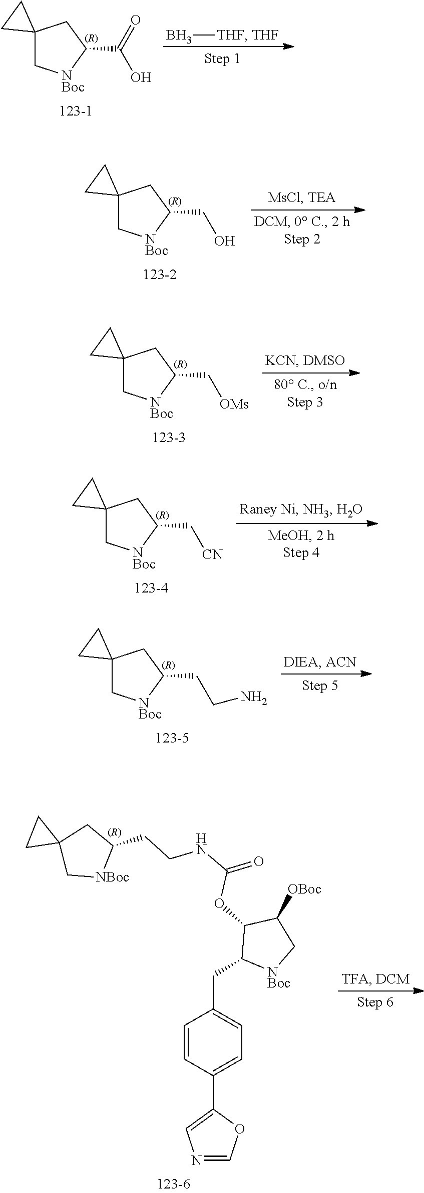

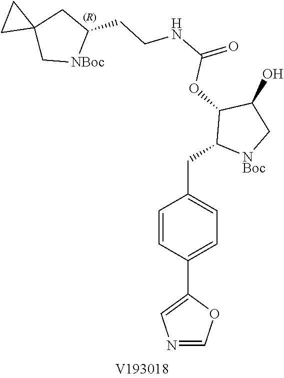

Step 1: To a stirred solution of (6R)-5-(tert-butoxycarbonyl)-5-azaspiro[2.4]heptane-6-carboxylic acid (123-1, 1 g, 4.14 mmol, 1 equiv) in THF (6 mL) were added Borane-tetrahydrofuran complex (1.0 M in THF) (6 mL) at 0° C. The resulting mixture was stirred at room temperature for overnight. The resulting mixture was concentrated under reduced pressure. The residue was purified by silica gel column chromatography, eluted with PE/EA (3:1) to afford tert-butyl (6R)-6-(hydroxymethyl)-5-azaspiro[2.4]heptane-5-carboxylate (930 mg, 98.7% yield, 95% purity) as a light yellow oil. MS: m/z: Calc'd for C₁₂H₂₁NO₃ [M+H−56]⁺172. Found, 172.

Step 2: To a stirred solution of tert-butyl (6R)-6-(hydroxymethyl)-5-azaspiro[2.4]heptane-5-carboxylate (123-2, 500 mg, 2.20 mmol, 1 equiv) and methanesulfonyl chloride (503.9 mg, 4.40 mmol, 2 equiv) in DCM (8 mL) were added TEA (667.7 mg, 6.60 mmol, 3 equiv) at 0° C. The resulting mixture was stirred at room temperature for 2 h. The resulting mixture was concentrated under reduced pressure. The residue was purified by silica gel column chromatography, eluted with PE/EA (3:1) to afford tert-butyl (6R)-6-[(methanesulfonyloxy)methyl]-5-azaspiro[2.4]heptane-5-carboxylate (600 mg, 89.32% yield) as a light yellow oil. MS: m/z: Calc'd for C₁₃H₂₃NO₅S [M+H−56]⁺250. Found, 250.

Step 3: To a stirred solution of tert-butyl (6R)-6-[(methanesulfonyloxy)methyl]-5-azaspiro[2.4]heptane-5-carboxylate (123-3, 400 mg, 1.31 mmol, 1 equiv) and KCN (255.8 mg, 3.93 mmol, 3 equiv) in DMSO (5 mL) at room temperature. The resulting mixture was stirred at 80° C. for overnight. The resulting mixture was extracted with EtOAc (3×100 mL). The combined organic layers were washed with brine (2×50 mL), dried over anhydrous Na₂SO₄. After filtration, the filtrate was concentrated under reduced pressure to afford tert-butyl (6R)-6-(cyanomethyl)-5-azaspiro[2.4]heptane-5-carboxylate (250 mg, 80.7% yield, 80% purity) as a white solid. MS: m/z: Calc'd for C₁₃H₂₀N₂O₂ [M+H−56]⁺181. Found, 181.

Step 4: To a stirred solution of tert-butyl (6R)-6-(cyanomethyl)-5-azaspiro[2.4]heptane-5-carboxylate (123-4, 245 mg, 1.03 mmol, 1 equiv) and raney nickel (182.5 mg, 3.11 mmol, 3 equiv) in methanol (5 mL) were added Ammonium hydroxide (9% in water) (36.3 mg, 1.03 mmol, 1 equiv) at room temperature. The resulting mixture was stirred at room temperature under hydrogen atmosphere for 2 h. The precipitated solids were collected by filtration and washed with methanol (4 mL) (2×10 mL). The resulting mixture was concentrated under reduced pressure to afford tert-butyl (6R)-6-(2-aminoethyl)-5-azaspiro[2.4]heptane-5-carboxylate (250 mg, 100.3% yield, 80% purity) as a light yellow oil. MS: m/z: Calc'd for C₁₃H₂₄N₂O₂ [M+H]⁺241. Found, 241.

### Step 5 and 6

The title compound was prepared in 41.7% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (R)-6-(2-aminoethyl)-5-azaspiro[2.4]heptane-5-carboxylate (123-5) in STEP 1; Boc Deprotection; General Procedure II in STEP 2. MS: m/z: Calc'd for C₂₃H₃₀N₄O₄ [M+H]⁺427. found 427. ¹H NMR (400 MHz, Methanol-d₄) δ 8.28 (s, 1H), 7.76 (d, J=8.2 Hz, 2H), 7.54 (s, 1H), 7.45 (d, J=8.0 Hz, 2H), 5.05-5.00 (m, 1H), 4.41 (d, J=4.1 Hz, 1H), 4.27 (s, 1H), 3.84 (p, J=7.6 Hz, 1H), 3.60 (dd, J=12.6, 4.2 Hz, 1H), 3.32 (d, J=15.3 Hz, 6H), 3.18-3.05 (m, 1H), 2.16-1.89 (m, 4H), 0.88-0.69 (m, 4H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min mL/min; Gradient: 2% B to 27% B in 10 min; Wave Length: 254 nm/220 nm; RT1(min): 8.5.

### Synthetic Procedure of (2R,3S,4S)-4-hydroxy-2-(4-(oxazol-5-yl)benzyl)pyrrolidin-3-yl ((1-(difluoromethyl)-1H-pyrazol-4-yl)methyl)carbamate (V193078)

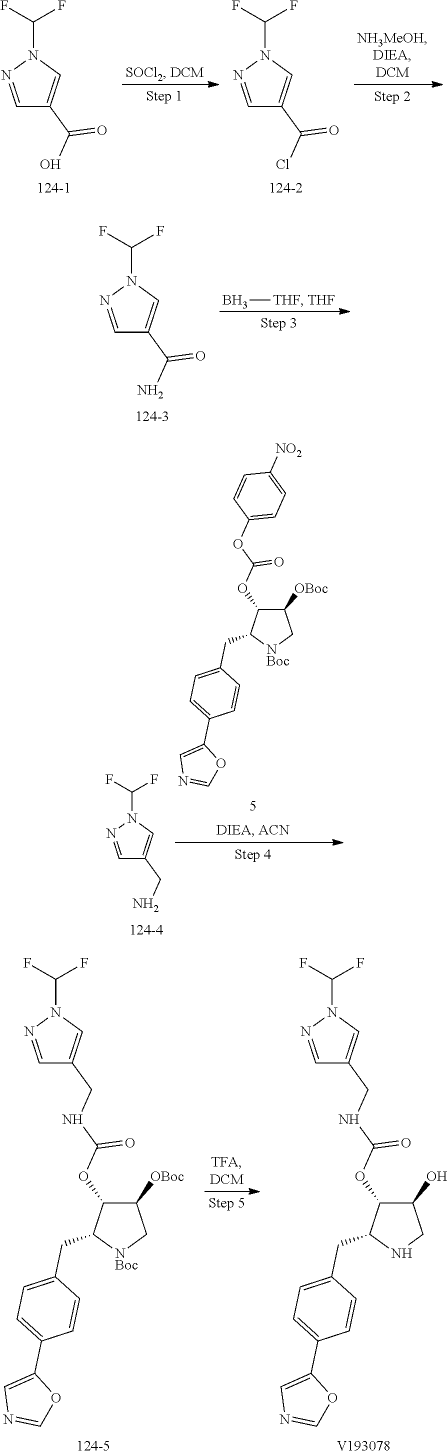

Step 1: To a stirred solution of 1-(difluoromethyl)pyrazole-4-carboxylic acid (124-1,100 mg, 0.617 mmol, 1 equiv) in DCM (3 mL) was added SOCl₂ (0.5 mL) at room temperature. The resulting mixture was stirred 2 h at 25° C. Upon completed. The resulting mixture was concentrated under reduced pressure. The resulting mixture was used in the next step directly without further purification. MS: m/z: Calc'd for C₅H₃ClF₂N₂O [M+H]⁺181. Found, 181.

Step 2: To a stirred solution of 1-(difluoromethyl)pyrazole-4-carbonyl chloride (124-2, 100 mg, 0.55 mmol, 1 equiv) in DCM (3 mL) was added DIEA (715.9 mg, 5.54 mmol, 10 equiv) and NH₃·MeOH (1.5 mL) at room temperature. The resulting mixture was stirred 2 h at 25° C. The resulting mixture was extracted with EtOAc (3×5 mL). The combined organic layers were washed with water (3×3 mL), dried over anhydrous Na₂SO₄. After filtration, the filtrate was concentrated under reduced pressure. The crude product was used in the next step directly without further purification. MS: m/z: Calc'd for C₅H₅F₂N₃O [M+H]⁺162. Found, 162.

Step 3: To a stirred solution of 1-(difluoromethyl)pyrazole-4-carboxamide (124-3, 100 mg, 0.62 mmol, 1 equiv) in THF (6 mL) was added Borane-tetrahydrofuran complex (1.0M in THF) (3 mL) in portions at 0° C. The resulting mixture was stirred 2 h at 25° C. under a nitrogen atmosphere. Desired product could be detected by LCMS. The resulting mixture was concentrated under reduced pressure.

The resulting mixture was used in the next step directly without further purification. MS: m/z: Calc'd for C₅H₇F₂N₃ [M+H]⁺148. Found, 148.

### Step 4 and 5

The title compound was prepared in 31.4% overall yield as a white solid according to Substitution reaction for synthesis of Carbamate; General Procedure I using tert-butyl (2S,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (1-(difluoromethyl)-1H-pyrazol-4-yl)methanamine (124-4) in STEP 1; Boc Deprotection; General Procedure II in STEP 2. MS: m/z: Calc'd for C₂₀H₂₁F₂N₅O₄[M+H]⁺434. Found, 434. ¹H NMR (400 MHz, Methanol-d₄) δ 8.28 (s, 1H), 8.03 (s, 1H), 7.76-7.68 (m, 3H), 7.54 (s, 1H), 7.50-7.31 (m, 3H), 4.98 (s, 1H), 4.41 (d, J=4.1 Hz, 1H), 4.34-4.20 (m, 3H), 3.59 (d, J=12.4 Hz, 1H), 3.22 (dd, J=23.7, 10.0 Hz, 2H), 3.13-3.03 (m, 1H).

Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 24% B in 10 min; Wave Length: 254 nm/220 nm; RT1(min): 9.78.

### Synthetic Procedure of (2R,3S,4S)-2-(4-(1-(difluoromethyl)-1H-pyrazol-4-yl)benzyl)-4-hydroxypyrrolidin-3-yl (3-fluorobenzyl)carbamate (V193079)

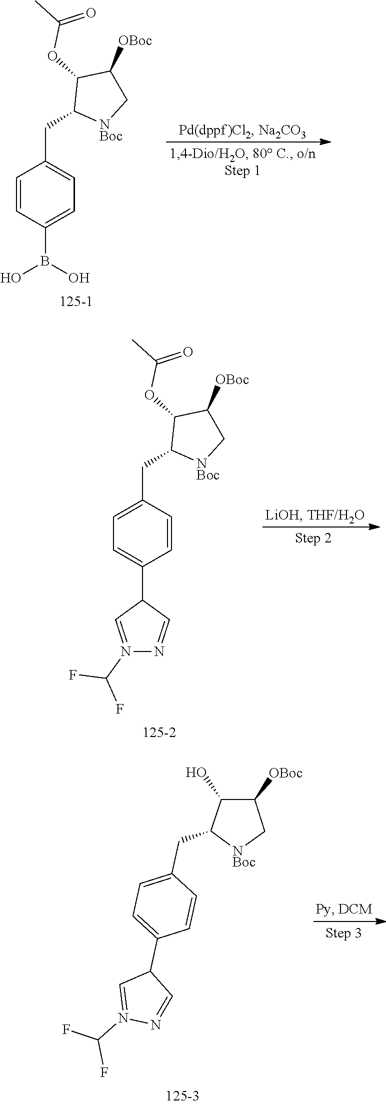

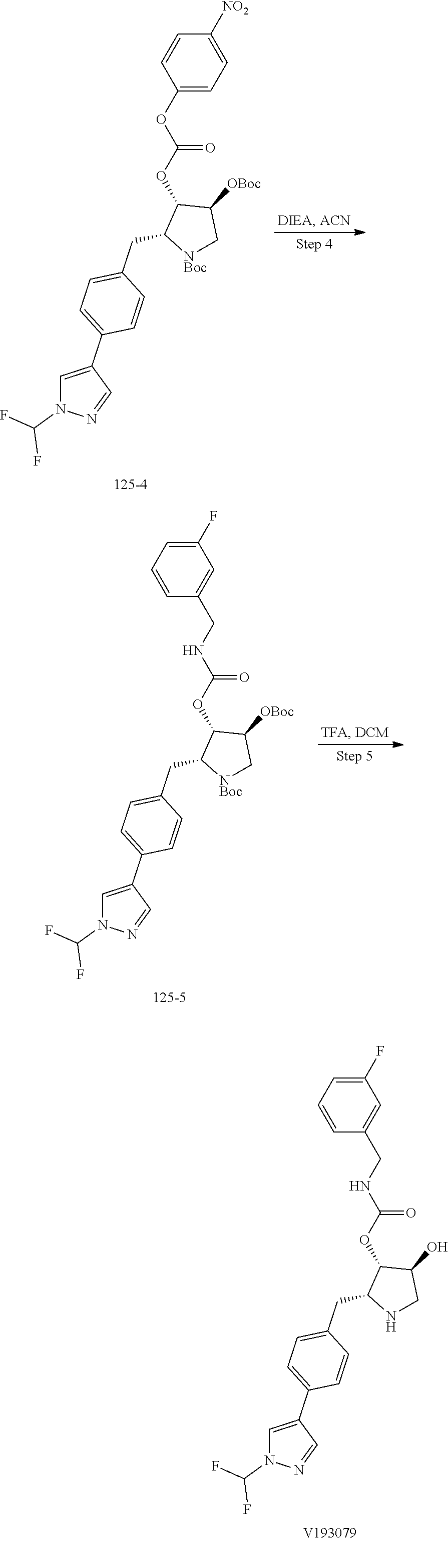

Step 1: To a stirred solution of 4-{[(2R,3S,4S)-3-(acetyloxy)-1-(tert-butoxycarbonyl)-4-[(tert-butoxycarbonyl)oxy]pyrrolidin-2-yl]methyl}phenylboronic acid (125-1,500 mg, 1.04 mmol, 1 equiv) and 1-(difluoromethyl)-4-iodopyrazole (509.0 mg, 2.08 mmol, 2 equiv) in 1,4-dioxane (5 mL) and H₂O (0.5 mL) was added Na₂CO₃ (331.6 mg, 3.13 mmol, 3 equiv) and Pd(dppf)Cl₂CH₂Cl₂ (85.2 mg, 0.10 mmol, 0.1 equiv) at room temperature. The reaction mixture was subsequently degassed by bubbling nitrogen through the solution for 5 minutes and then stirred at 80° C. for overnight. After completion of the reaction, the reaction mixture was quenched with water 100 mL. The aqueous layer was extracted with ethyl acetate (100 mL×2). Then, the combined organic phase was washed with brine, dried over anhydrous Na₂SO₄. Purified by column chromatography on silica gel (Combiflash) (PE/EtOAc=3:1) to afford tert-butyl (2R,3S,4S)-3-(acetyloxy)-4-[(tert-butoxycarbonyl)oxy]-2-({4-[1-(difluoromethyl)pyrazol-4-yl]phenyl}methyl)pyrrolidine-1-carboxylate (300 mg, 52.14% yield, 95% purity) as a light yellow solid. MS: m/z: Calc'd for C₂₇H₃₅F₂N₃O₇[M−56-56]⁺ 440. found 440.

Step 2: To a stirred solution of tert-butyl (2R,3S,4S)-3-(acetyloxy)-4-[(tert-butoxycarbonyl)oxy]-2-({4-[1-(difluoromethyl)pyrazol-4-yl]phenyl}methyl)pyrrolidine-1-carboxylate (124-2, 300 mg, 0.54 mmol, 1 equiv) in THF (5 mL) was added LiOH (65.1 mg, 2.72 mmol, 5 equiv) in H₂O (1 mL) dropwise at 0° C. under nitrogen atmosphere and stirred at room temperature for overnight. The reaction progress was monitored by TLC. After completion of reaction, the reaction mixture was diluted with water (100 mL), the resulting mixture was extracted with EtOAc (1×100 mL). The combined organic layers were dried over anhydrous Na₂SO₄. After filtration, the filtrate was concentrated under reduced pressure to give crude product tert-butyl (2R,3S,4S)-4-[(tert-butoxycarbonyl)oxy]-2-({4-[1-(difluoromethyl)pyrazol-4-yl]phenyl}methyl)-3-hydroxypyrrolidine-1-carboxylate (250 mg, 90.21% yield, 89% purity) as a light yellow solid. MS: m/z: Calc'd for C₂₅H₃₃F₂N₃O₆ [M−56−56]⁺ 398. found 398.

Step 3: To a stirred solution of tert-butyl (2R,3S,4S)-4-[(tert-butoxycarbonyl)oxy]-2-({4-[1-(difluoromethyl)pyrazol-4-yl]phenyl}methyl)-3-hydroxypyrrolidine-1-carboxylate (125-3, 250 mg, 0.49 mmol, 1 equiv) in anhydrous DCM (5 mL) was added pyridine (116.4 mg, 1.47 mmol, 3 equiv) and 4-nitrophenyl carbonochloridate (197.8 mg, 0.98 mmol, 2 equiv) and stirred at room temperature for 2 h. The reaction progress was monitored by TLC. After completion of reaction, the reaction mixture was concentrated under reduced pressure to give crude product which was further purified by silica gel column chromatography, eluted with PE/EA (4:1) to afford tert-butyl (2R,3S,4S)-4-[(tert-butoxycarbonyl)oxy]-2-({4-[1-(difluoromethyl)pyrazol-4-yl]phenyl}methyl)-3-[(4-nitrophenoxycarbonyl)oxy]pyrrolidine-1-carboxylate (300 mg, 90.63% yield, 95% purity) as an off-white solid. MS: m/z: Calc'd for C₃₂H₃₆F₂N₄O₁₀ [M−56]⁺ 619. found 619.

Step 4 and 5: The title compound was prepared in 63.5% overall yield as a light yellow solid according to Substitution reaction for synthesis of Carbamate; General Procedure I using tert-butyl (2R,3S,4S)-4-((tert-butoxycarbonyl)oxy)-2-(4-(1-(difluoromethyl)-1H-pyrazol-4-yl)benzyl)-3-(((4-nitrophenoxy)carbonyl)oxy)pyrrolidine-1-carboxylate (125-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (3-fluorophenyl)methanamine in STEP 4; Boc Deprotection; General Procedure II in STEP 5. MS: m/z: Calc'd for C₂₃H₂₃F₃N₄O₃[M+H]⁺461. found 461. ¹H NMR (400 MHz, Methanol-d4) δ 8.41 (s, 1H), 8.08 (s, 1H), 7.71-7.30 (m, 6H), 7.18 (t, J=8.6 Hz, 1H), 7.11 (dt, J=9.8, 2.2 Hz, 1H), 7.04 (td, J=8.5, 2.6 Hz, 1H), 4.97 (d, J=3.5 Hz, 1H), 4.52-4.28 (m, 3H), 4.25 (td, J=7.9, 3.6 Hz, 1H), 3.61 (dd, J=12.7, 4.3 Hz, 1H), 3.29-3.15 (m, 2H), 3.08 (dd, J=14.0, 8.2 Hz, 1H).

Prep-HPLC conditions: Column: Xselect CSH Prep C18 Column, 30*250 mm, 10 μm; Mobile Phase A: Water (0.05% TFA), Mobile Phase B: ACN; Flow rate: 60 mL/min mL/min; Gradient: 18% B to 48% B in 10 min; Wave Length: 254 nm/220 nm; RT1(min): 8.5.

### Synthetic Procedure of (2R,3S,4S)-2-(4-(1-(difluoromethyl)-1H-pyrazol-4-yl)benzyl)-4-hydroxypyrrolidin-3-yl (2-((S)-pyrrolidin-2-yl)ethyl)carbamate (V193080)

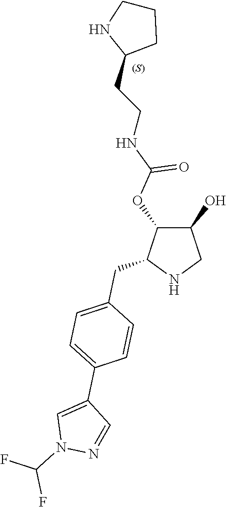

The title compound was prepared in 25.7% overall yield as an off-white solid according to Substitution reaction for synthesis of Carbamate; General Procedure I using tert-butyl (2R,3S,4S)-4-((tert-butoxycarbonyl)oxy)-2-(4-(1-(difluoromethyl)-1H-pyrazol-4-yl)benzyl)-3-(((4-nitrophenoxy)carbonyl)oxy)pyrrolidine-1-carboxylate (125-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (S)-2-(2-aminoethyl)pyrrolidine-1-carboxylate in STEP 1; Boc Deprotection; General Procedure II in STEP 2. MS: m/z: Calc'd for C₂₂H₂₉F₂N₅O₃[M+H]⁺450, found 450.

¹H NMR (400 MHz, Methanol-d4) δ 8.44 (s, 1H), 8.10 (s, 1H), 7.77-7.61 (m, 2H), 7.54-7.31 (m, 3H), 5.01 (s, 1H), 4.40 (d, J=4.3 Hz, 1H), 4.25 (s, 1H), 3.73-3.47 (m, 2H), 3.32-3.16 (m, 6H), 3.15-2.98 (m, 1H), 2.37-2.22 (m, 1H), 2.11 (d, J=9.9 Hz, 1H), 2.08-1.90 (m, 3H), 1.79-1.65 (m, 1H), 1.32 (s, OH).

Prep-HPLC conditions: Column: Column: Xselect CSH Prep C18 OBD Column, 30*150 mm, 5 μm; Mobile Phase A: Water (0.05% TFA), Mobile Phase B: ACN; Flow rate: 60 mL/min mL/min; Gradient: 3% B to 26% B in 7 min; Wave Length: 254 nm/220 nm; RT1(min): 6.3

### Synthetic Procedure of (2R,3S,4S)-2-(4-(1-(difluoromethyl)-1H-pyrazol-4-yl)benzyl)-4-hydroxypyrrolidin-3-yl (2-((S)-pyrrolidin-3-yl)ethyl)carbamate (V193081)

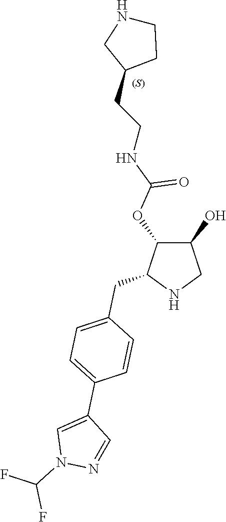

The title compound was prepared in 41.2% overall yield as a white solid according to Substitution reaction for synthesis of Carbamate; General Procedure I using tert-butyl (2R,3S,4S)-4-((tert-butoxycarbonyl)oxy)-2-(4-(1-(difluoromethyl)-1H-pyrazol-4-yl)benzyl)-3-(((4-nitrophenoxy)carbonyl)oxy)pyrrolidine-1-carboxylate (125-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (R)-3-(2-aminoethyl)pyrrolidine-1-carboxylate in STEP 1; Boc Deprotection; General Procedure II in STEP 2. MS: m/z: Calc'd for C₂₂H₂₉F₂N₅O₃[M+H]⁺450, found 450. ¹H NMR (400 MHz, Methanol-d4) δ 8.43 (d, J=0.7 Hz, 1H), 8.10 (s, 1H), 7.72-7.60 (m, 2H), 7.55-7.32 (m, 3H), 5.01-4.95 (m, 1H), 4.39 (dd, J=4.0, 1.4 Hz, 1H), 4.28-4.16 (m, 1H), 3.60 (dd, J=12.6, 4.3 Hz, 1H), 3.50 (dd, J=11.5, 7.5 Hz, 1H), 3.45-3.35 (m, 1H), 3.32-3.15 (m, 5H), 3.08 (dd, J=14.1, 8.5 Hz, 1H), 2.88 (dd, J=11.6, 9.1 Hz, 1H), 2.45-2.29 (m, 1H), 2.29-2.19 (m, 1H), 1.81-1.60 (m, 3H).

Prep-HPLC conditions: Column: Xselect CSH Prep C18 OBD Column, 30*150 mm, 5 μm; Mobile Phase A: Water (0.05% TFA), Mobile Phase B: ACN; Flow rate: 60 mL/min mL/min; Gradient: 3% B to 25% B in 7 min; Wave Length: 254 nm/220 nm; RT1(min): 6.4.

### Synthetic Procedure of (2R,3S,4S)-2-(4-(4-cyanothiophene-2-yl)benzyl)-4-hydroxypyrrolidin-3-yl (3-fluorobenzyl)carbamate (V193129)

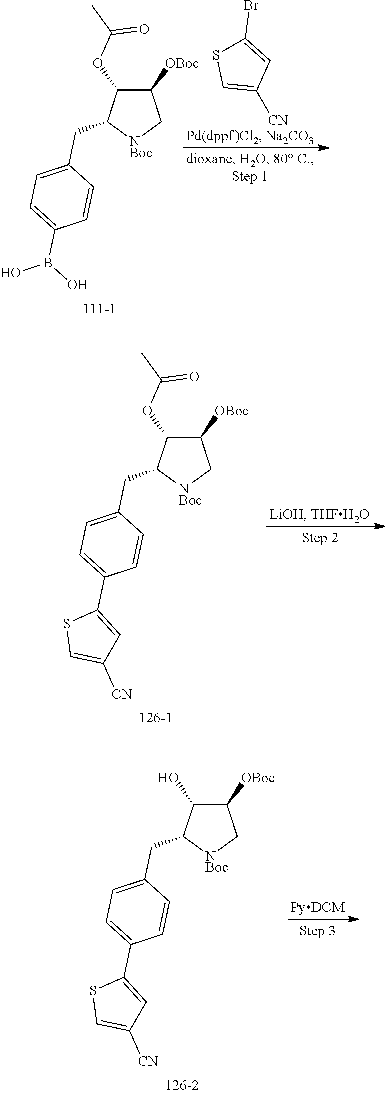

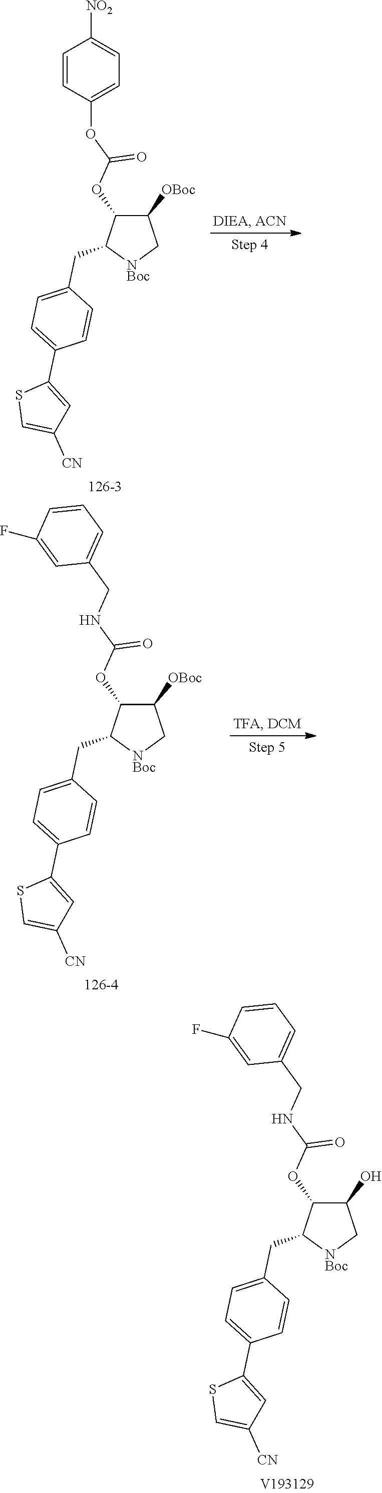

Step 1: To a solution of 4-{[(2R,3S,4S)-3-(acetyloxy)-1-(tert-butoxycarbonyl)-4-[(tert-butoxycarbonyl)oxy]pyrrolidin-2-yl]methyl}phenylboronic acid (111-1, 200 mg, 0.41 mmol, 1 equiv) and 5-bromothiophene-3-carbonitrile (78.4 mg, 0.41 mmol, 1 equiv) in Dioxane (3 mL) and H₂O (0.3 mL) was added Na₂CO₃ (88.4 mg, 0.83 mmol, 2 equiv) and Pd(dppf)Cl₂CH₂Cl₂ (68.1 mg, 0.08 mmol, 0.2 equiv). The mixture was stirred at 80° C. for o/n under N₂. The mixture was extracted with EA and washed with water, then brine. The organic layer was dried over sodium sulfate, concentrated. The residue was purified by Prep-TLC to obtain tert-butyl (2R,3S,4S)-3-(acetyloxy)-4-[(tert-butoxycarbonyl)oxy]-2-{[4-(4-cyanothiophene-2-yl)phenyl]methyl}pyrrolidine-1-carboxylate (160 mg, 70.6% yield, 90% purity) as a yellow solid. MS: m/z: Calc'd for C₂₈H₃₄N₂O₇S [M+H]⁺ 543 Found, 543.

Step 2: To a solution of tert-butyl (2R,3S,4S)-3-(acetyloxy)-4-[(tert-butoxycarbonyl)oxy]-2-{[4-(4-cyanothiophene-2-yl)phenyl]methyl}pyrrolidine-1-carboxylate (126-1, 160 mg, 0.29 mmol, 1 equiv) in THF (3 mL) and H₂O (0.3 mL) was added LiOH (14.1 mg, 0.59 mmol, 2 equiv). The mixture was stirred at r.t. for o/n. The mixture was diluted with EA and washed with water, then brine.

The organic layer was dried over sodium sulfate, concentrated. The crude product tert-butyl (2R,3S,4S)-4-[(tert-butoxycarbonyl)oxy]-2-{[4-(4-cyanothiophene-2-yl)phenyl]methyl}-3-hydroxypyrrolidine-1-carboxylate (130 mg, 88.0% yield, 70% purity) was used directly in the next step without further purification. MS: m/z: Calc'd for C₂₆H₃₂N₂O₆S [M+H]⁺ 501 Found, 501.

Step 3: To a solution of tert-butyl (2R,3S,4S)-4-[(tert-butoxycarbonyl)oxy]-2-{[4-(4-cyanothiophene-2-yl)phenyl]methyl}-3-hydroxypyrrolidine-1-carboxylate (126-2, 120 mg, 0.24 mmol, 1 equiv) and 4-nitrophenyl carbonochloridate (72.4 mg, 0.36 mmol, 1.5 equiv) in DCM (5 mL) was added pyridine (37.9 mg, 0.48 mmol, 2 equiv) at 0° C. The reaction was stirred at r.t. for o/n. The mixture was diluted with water and extracted with EA, washed with brine. The organic layer was dried over sodium sulfate, concentrated. The residue was purified by reversed phase column to obtain tert-butyl (2R,3S,4S)-4-[(tert-butoxycarbonyl)oxy]-2-{[4-(4-cyanothiophene-2-yl)phenyl]methyl}-3-[(4-nitrophenoxycarbonyl)oxy]pyrrolidine-1-carboxylate (60 mg, 37.6% yield, 100% purity) as a yellow oil. MS: m/z: Calc'd for C₃₃H₃₅N₃O₁₀S [M+H]⁺ 666 Found, 666.

Step 4 and 5: The title compound was prepared in 65.6% overall yield as a white solid according to Substitution reaction for synthesis of Carbamate; General Procedure I using tert-butyl (2R,3S,4S)-4-((tert-butoxycarbonyl)oxy)-2-(4-(4-cyanothiophene-2-yl)benzyl)-3-(((4-nitrophenoxy)carbonyl)oxy)pyrrolidine-1-carboxylate (126-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (3-fluorophenyl)methanamine in STEP 4; Boc Deprotection; General Procedure II in STEP 5. MS: m/z: Calc'd for C₂₄H₂₂FN₃O₃S [M+H]⁺ 452 Found, 452.

¹H NMR (400 MHz, Methanol-d4) δ 8.24 (d, J=1.5 Hz, 1H), 7.69-7.62 (m, 3H), 7.44-7.34 (m, 3H), 7.25-7.00 (m, 3H), 4.97 (s, 1H), 4.45-4.36 (m, 2H), 4.36-4.22 (m, 2H), 3.65-3.56 (m, 1H), 3.31-3.17 (m, 2H), 3.12 (d, J=7.6 Hz, 1H).

Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 16% B to 46% B in 10 min; Wave Length: 254 nm/220 nm; RT1(min): 9.45.

### Synthetic Procedure of (2R,3S,4S)-2-(4-(difluoromethyl)benzyl)-4-hydroxypyrrolidin-3-yl (isoindolin-4-ylmethyl)carbamate (V193139)

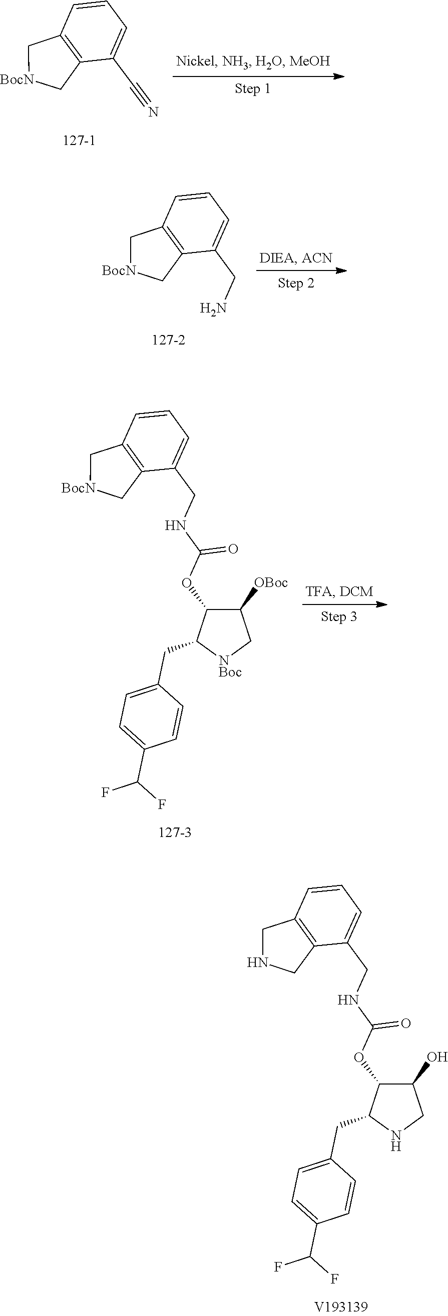

Step 1: To a stirred solution of tert-butyl 4-cyano-1,3-dihydroisoindole-2-carboxylate (127-1, 100 mg, 0.41 mmol, 1 equiv) in methanol (5 mL) was added Nickel (72.1 mg, 1.23 mmol, 3 equiv) and NH₃·H₂O (14.4 mg, 0.41 mmol, 1 equiv) at room temperature under H₂. The resulting mixture was stirred overnight at 25° C. under H₂. The resulting mixture was filtered, the filter cake was washed with MeOH (2×1 mL). The filtrate was concentrated under reduced pressure. This resulted in tert-butyl 4-(aminomethyl)-1,3-dihydroisoindole-2-carboxylate (70 mg) as a light yellow oil. MS: m/z: Calc'd for C₁₄H₂₀N₂O₂ [M+H−56+41]⁺234. Found, 234.

Step 2 and 3: The title compound was prepared in 70.5% overall yield as a white solid according to Substitution reaction for synthesis of Carbamate; General Procedure I using tert-butyl (2R,3S,4S)-4-((tert-butoxycarbonyl)oxy)-2-(4-(difluoromethyl)benzyl)-3-(((4-nitrophenoxy)carbonyl)oxy)pyrrolidine-1-carboxylate (85-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 4-(aminomethyl)isoindoline-2-carboxylate (126-2) in STEP 2; Boc Deprotection; General Procedure II in STEP 3. MS: m/z: Calc'd for C₂₂H₂₅F₂N₃O₃[M+H]⁺418. Found, 418. ¹H NMR (400 MHz, Methanol-d₄) δ 7.54-7.26 (m, 7H), 6.92-6.61 (m, 1H), 4.95 (s, 1H), 4.76 (s, 2H), 4.66 (s, 2H), 4.40 (d, J=4.7 Hz, 1H), 4.35 (s, 2H), 4.29-4.20 (m, 1H), 3.61 (dd, J=12.6, 4.2 Hz, 1H), 3.28-3.19 (m, 2H), 3.13 (dd, J=14.0, 8.6 Hz, 1H).

Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 2% B to 24% B in 10 min; Wave Length: 254 nm/220 nm; RT1(min): 9.78.

### Synthetic Procedure of (2R,3S,4S)-2-(4-(4-(difluoromethyl)thiophen-2-yl)benzyl)-4-hydroxypyrrolidin-3-yl (3-fluorobenzyl)carbamate (V193142)

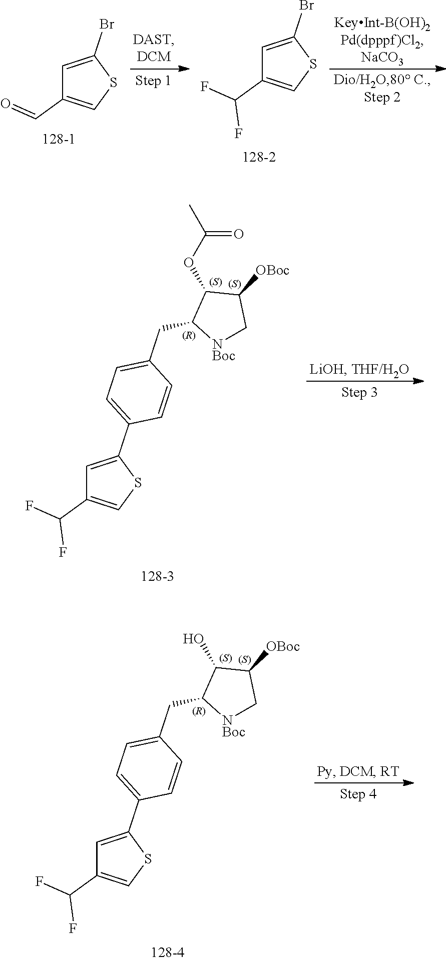

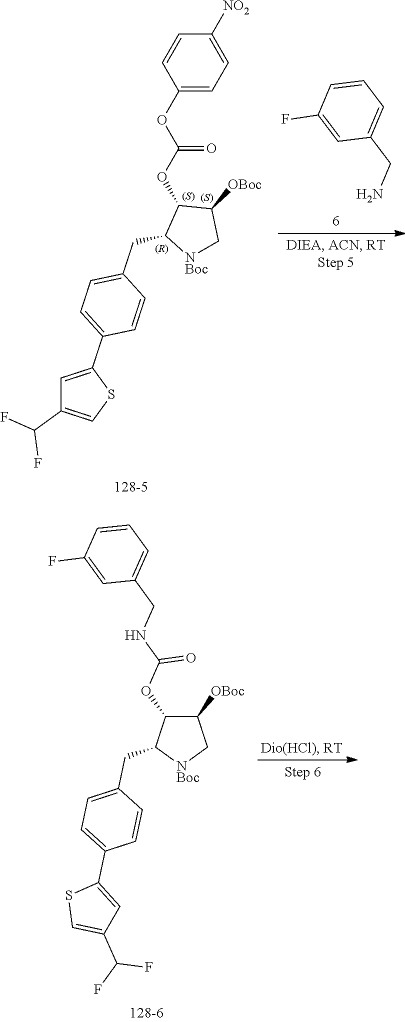

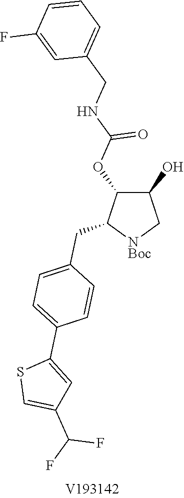 [SMILES: CC(C)(C)OC(=O)N1C[C@H](O)[C@@H](OC(=O)NCc2cccc(F)c2)[C@H]1Cc1ccc(-c2cc(C(F)F)cs2)cc1]

Step 1: To a stirred solution of 5-bromothiophene-3-carbaldehyde (128-1, 2 g, 10.46 mmol, 1 equiv) in anhydrous DCM (20 mL) was added Diethylaminosulfur trifluoride (4.2 mL, 31.39 mmol, 3.00 equiv) at 0° C. and stirred at room temperature for 3 h. The reaction progress was monitored by TLC. After completion of reaction, the reaction mixture was poured into sat. sodium bicarbonate (aq.) at 0° C. The resulting mixture was extracted with CH₂Cl₂ (2×100 mL). The combined organic layers were dried over anhydrous Na₂SO₄. After filtration, the filtrate was concentrated under reduced pressure to give crude product which was further purified by silica gel column chromatography, eluted with PE/EA (4:1) to afford 2-bromo-4-(difluoromethyl)thiophene (1.3 g, 58.29% yield, 100% purity) as a colorless oil. ¹H NMR (400 MHz, Chloroform-d) δ 7.68 (t, J=1.8 Hz, 1H), 6.86-6.38 (m, 2H).

Step 2: To a degassed solution of 4-{[(2R,3S,4S)-3-(acetyloxy)-1-(tert-butoxycarbonyl)-4-[(tert-butoxycarbonyl)oxy]pyrrolidin-2-yl]methyl}phenylboronic acid (1 g, 2.08 mmol, 1 equiv) and 2-bromo-4-(difluoromethyl)thiophene (128-2, 0.89 g, 4.17 mmol, 2 equiv) in 1,4-dioxane (20 mL) and H₂O (2 mL) was added Na₂CO₃ (0.66 g, 6.25 mmol, 3 equiv) and Pd(dppf)Cl₂ (0.31 g, 0.417 mmol, 0.2 equiv) at room temperature. The reaction mixture was subsequently degassed by bubbling nitrogen through the solution for 5 minutes and then stirred at 80° C. for overnight. After completion of the reaction, the reaction mixture was quenched with water 200 mL. The aqueous layer was extracted with ethyl acetate (200 mL×2). Then, the combined organic phase was washed with brine, dried over anhydrous Na₂SO₄ and purified by column chromatography on silica gel (Combiflash) (PE/EtOAc=3:1) to afford tert-butyl (2R,3S,4S)-3-(acetyloxy)-4-[(tert-butoxycarbonyl)oxy]-2-({4-[4-(difluoromethyl)thiophen-2-yl]phenyl}methyl)pyrrolidine-1-carboxylate (700 mg, 59.11% yield, 87% purity) as a light yellow solid. MS: m/z: Calc'd for C₂₈H₃₅F₂NO₇S [M−56−56]⁺ 456. found 456.

Step 3: To a stirred solution of tert-butyl (2R,3S,4S)-3-(acetyloxy)-4-[(tert-butoxycarbonyl)oxy]-2-({4-[4-(difluoromethyl)thiophen-2-yl]phenyl}methyl)pyrrolidine-1-carboxylate (128-3, 700 mg, 1.23 mmol, 1 equiv) in THF (10 mL) was added LiOH (147.6 mg, 6.16 mmol, 5 equiv) in H₂O (2 mL) dropwise at 0° C. under nitrogen atmosphere and stirred at room temperature for overnight. The reaction progress was monitored by TLC. After completion of reaction, the reaction mixture was diluted with water (100 mL), the resulting mixture was extracted with EtOAc (2×150 mL). The combined organic layers were dried over anhydrous Na₂SO₄. After filtration, the filtrate was concentrated under reduced pressure to give crude product tert-butyl (2R,3S,4S)-4-[(tert-butoxycarbonyl)oxy]-2-({4-[4-(difluoromethyl)thiophen-2-yl]phenyl}methyl)-3-hydroxypyrrolidine-1-carboxylate (600 mg) as a light yellow solid. MS: m/z: Calc'd for C₂₆H₃₃F₂NO₆S [M−56−56]⁺ 414. found 414.

Step 4: To a stirred solution of tert-butyl (2R,3S,4S)-4-[(tert-butoxycarbonyl)oxy]-2-({4-[4-(difluoromethyl)thiophen-2-yl]phenyl}methyl)-3-hydroxypyrrolidine-1-carboxylate (128-4, 500 mg, 0.95 mmol, 1 equiv) in anhydrous DCM (10 mL) was added pyridine (225.7 mg, 2.85 mmol, 3 equiv) and 4-nitrophenyl carbonochloridate (383.4 mg, 1.90 mmol, 2 equiv) and stirred at room temperature for 2 h. The reaction progress was monitored by TLC. After completion of reaction, the reaction mixture was concentrated under reduced pressure to give crude product which was further purified by silica gel column chromatography, eluted with PE/EA (4:1) to afford tert-butyl (2R,3S,4S)-4-[(tert-butoxycarbonyl)oxy]-2-({4-[4-(difluoromethyl)thiophen-2-yl]phenyl}methyl)-3-[(4-nitrophenoxycarbonyl)oxy]pyrrolidine-1-carboxylate (500 mg, 76.10% yield, 93% purity) as a light yellow solid. MS: m/z: Calc'd for C₃₃H₃₆F₂N₂O₁₀S [M−100]⁺ 591. found 591.

### Step 5 and 6

The title compound was prepared in 45.5% overall yield as a white solid according to Substitution reaction for synthesis of Carbamate; General Procedure I using tert-butyl (2R,3S,4S)-4-((tert-butoxycarbonyl)oxy)-2-(4-(4-(difluoromethyl)thiophen-2-yl)benzyl)-3-(((4-nitrophenoxy)carbonyl)oxy)pyrrolidine-1-carboxylate (127-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), (3-fluorophenyl)methanamine in STEP 5; Boc Deprotection; General Procedure II in STEP 6. MS: m/z: Calc'd for C₂₄H₂₃F₃N₂O₃S [M+H]⁺ 477. found 477. ¹H NMR (400 MHz, Methanol-d4) δ 7.71 (q, J=1.8 Hz, 1H), 7.66-7.60 (m, 2H), 7.51 (s, 1H), 7.43-7.32 (m, 3H), 7.17 (d, J=7.7 Hz, 1H), 7.11 (d, J=9.8 Hz, 1H), 7.07-7.01 (m, 1H), 6.81 (t, J=56.1 Hz, 1H), 4.97 (d, J=3.4 Hz, 1H), 4.45-4.31 (m, 3H), 4.31-4.20 (m, 1H), 3.60 (dd, J=12.6, 4.3 Hz, 1H), 3.29-3.16 (m, 2H), 3.08 (dd, J=13.9, 8.2 Hz, 1H).

Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 30% B to 40% B in 10 min; Wave Length: 254 nm/220 nm; RT1(min): 9.87.

### Synthetic Procedure of (2R,3S,4S)-2-(4-(4-(difluoromethyl)thiophen-2-yl)benzyl)-4-hydroxypyrrolidin-3-yl (isoindolin-5-ylmethyl)carbamate (V193165)

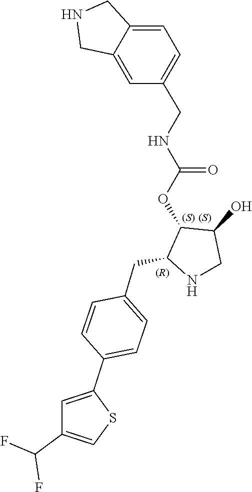

The title compound was prepared in 36.1% overall yield as an off-white solid according to Substitution reaction for synthesis of Carbamate; General Procedure I using tert-butyl (2R,3S,4S)-4-((tert-butoxycarbonyl)oxy)-2-(4-(4-(difluoromethyl)thiophen-2-yl)benzyl)-3-(((4-nitrophenoxy)carbonyl)oxy)pyrrolidine-1-carboxylate (127-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 5-(aminomethyl)isoindoline-2-carboxylate in STEP 1; Boc Deprotection; General Procedure II in STEP 2. MS: m/z: Calc'd for C₂₆H₂₇F₂N₃O₃S [M+H]⁺ 500, found 500. ¹H NMR (400 MHz, Methanol-d4) δ 7.72 (q, J=1.8 Hz, 1H), 7.68-7.61 (m, 2H), 7.55-7.50 (m, 1H), 7.50-7.38 (m, 5H), 6.82 (t, J=56.0 Hz, 1H), 5.03-4.97 (m, 1H), 4.63 (d, J=4.0 Hz, 4H), 4.46-4.29 (m, 3H), 4.29-4.15 (m, 1H), 3.65 (dd, J=12.7, 4.3 Hz, 1H), 3.27-3.17 (m, 2H), 3.12 (dd, J=14.0, 8.5 Hz, 1H).

Prep-HPLC conditions: Column: Xselect CSH Prep C18 Column, 30*150 mm, 5 μm; Mobile Phase A: Water (0.05% HCL), Mobile Phase B: ACN; Flow rate: 60 mL/min mL/min; Gradient: 5% B to 35% B in 10 min; Wave Length: 254 nm/220 nm; RT1(min): 9.23

### Synthetic Procedure of (2R,3S,4S)-2-(4-(4-(difluoromethyl)thiophen-2-yl)benzyl)-4-hydroxypyrrolidin-3-yl (2-((S)-pyrrolidin-2-yl)ethyl)carbamate (V193238)

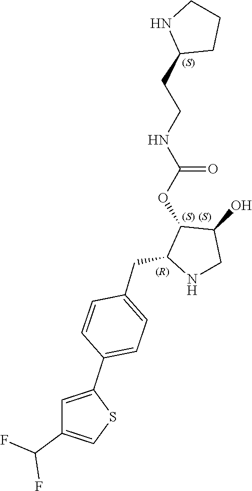

The title compound was prepared in 34.1% overall yield as an off-white solid according to Substitution reaction for synthesis of Carbamate; General Procedure I using tert-butyl (2R,3S,4S)-4-((tert-butoxycarbonyl)oxy)-2-(4-(4-(difluoromethyl)thiophen-2-yl)benzyl)-3-(((4-nitrophenoxy)carbonyl)oxy)pyrrolidine-1-carboxylate (127-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (S)-2-(2-aminoethyl)pyrrolidine-1-carboxylate in STEP 1; Boc Deprotection; General Procedure II in STEP 2. MS: m/z: Calc'd for C₂₃H₂₉F₂N₃O₃S [M+H]⁺ 466, found 466. ¹H NMR (400 MHz, Methanol-d4) δ 7.74-7.66 (m, 3H), 7.55-7.39 (m, 3H), 6.81 (t, J=56.0 Hz, 1H), 5.01 (s, 1H), 4.41 (d, J=4.1 Hz, 1H), 4.26 (s, 1H), 3.67-3.53 (m, 2H), 3.33 (s, 2H), 3.32-3.04 (m, 5H), 2.37-2.24 (m, 1H), 2.22-1.90 (m, 4H), 1.78-1.62 (m, 1H).

Prep-HPLC conditions: Column: Xselect CSH Prep C18 Column, 30*150 mm, 5 μm; Mobile Phase A: Water (0.05% HCL), Mobile Phase B: ACN; Flow rate: 60 mL/min mL/min; Gradient: 8% B to 38% B in 10 min; Wave Length: 254 nm/220 nm; RT1(min): 8.99.

### Synthetic Procedure of (2R,3S,4S)-2-(4-(4-(difluoromethyl)thiophen-2-yl)benzyl)-4-hydroxypyrrolidin-3-yl (3-(azetidin-3-yl)benzyl)carbamate (V193243)

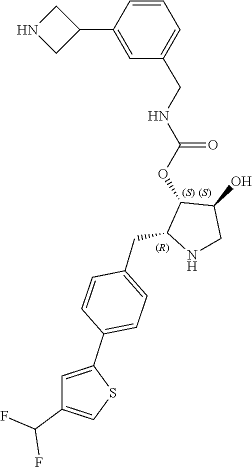

The title compound was prepared in 30.1% overall yield as an white solid according to Substitution reaction for synthesis of Carbamate; General Procedure I using tert-butyl (2R,3S,4S)-4-((tert-butoxycarbonyl)oxy)-2-(4-(4-(difluoromethyl)thiophen-2-yl)benzyl)-3-(((4-nitrophenoxy)carbonyl)oxy)pyrrolidine-1-carboxylate (127-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 3-(3-(aminomethyl)phenyl)azetidine-1-carboxylate in STEP 1; Boc Deprotection; General Procedure II in STEP 2. MS: m/z: Calc'd for C₂₇H₂₉F₂N₃O₃S [M+H]⁺ 514, found 514.

¹H NMR (400 MHz, Methanol-d4) δ 7.71 (d, J=2.2 Hz, 1H), 7.56 (d, J=8.0 Hz, 2H), 7.52-7.08 (m, 7H), 6.81 (t, J=56.1 Hz, 1H), 4.96 (d, J=3.5 Hz, 1H), 4.52-4.12 (m, 9H), 3.63 (dd, J=12.6, 4.3 Hz, 1H), 3.29-2.86 (m, 3H).

Prep-HPLC conditions: Column: Xselect CSH Prep C18 Column, 30*150 mm, 5 μm; Mobile Phase A: Water (0.05% TFA), Mobile Phase B: ACN; Flow rate: 60 mL/min mL/min; Gradient: 4% B to 34% B in 10 min; Wave Length: 254 nm/220 nm; RT1(min): 9.8

### Synthetic Procedure of (2R,3S,4S)-4-hydroxy-2-{[4-(1,3,4-thiadiazol-2-yl)phenyl]methyl}pyrrolidin-3-yl N-[(3-fluorophenyl)methyl]carbamate (V192411)

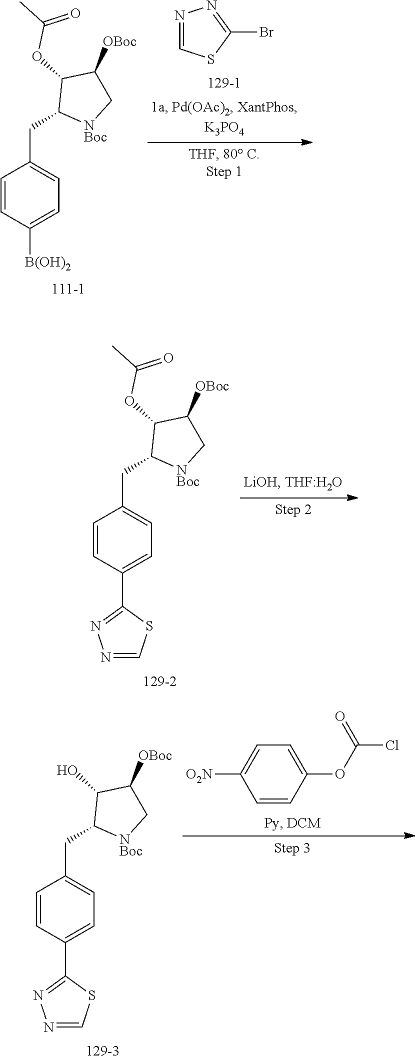

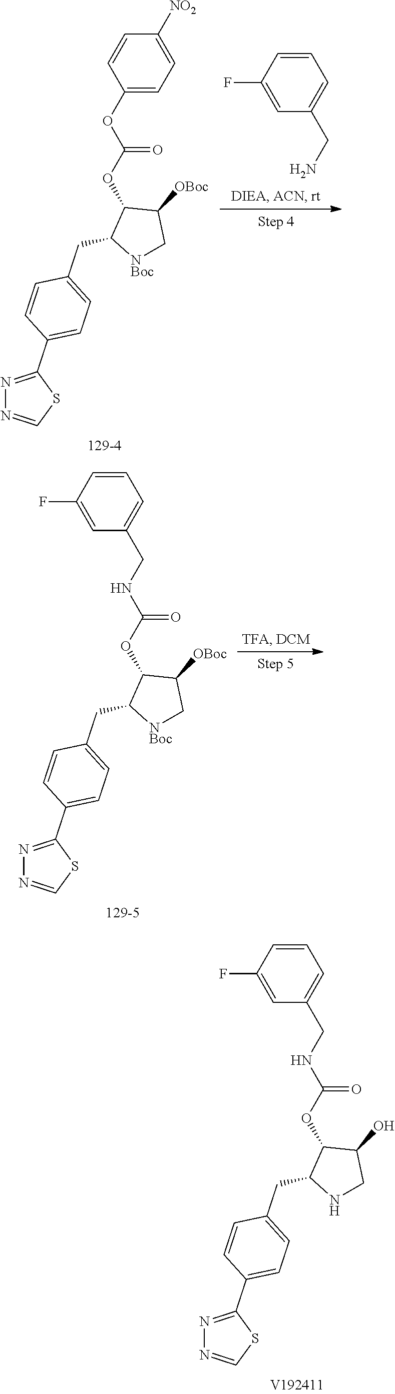

Step 1: To the solution of 4-{[(2R,3S,4S)-3-(acetyloxy)-1-(tert-butoxycarbonyl)-4-[(tert-butoxycarbonyl)oxy]pyrrolidin-2-yl]methyl}phenylboronic acid (100 mg, 0.21 mmol, 1 equiv.) and 2-bromo-1,3,4-thiadiazole (34 mg, 0.21 mmol, 1.5 equiv.) in THF (3 mL) were added Pd(OAc)₂ (3 mg, 0.014 mmol, 0.1 equiv.), XantPhos (16 mg, 0.028 mmol, 0.2 equiv.) and K₃PO4 (59 mg, 0.28 mmol, 2 equiv.). The mixture was stirred at 80° C. overnight under N₂. The mixture was filtered and the filtrate was concentrated. The crude product was purified by Prep TLC (PE:EA=1:1) to afford tert-butyl (2R,3S,4S)-3-(acetyloxy)-4-[(tert-butoxycarbonyl)oxy]-2-{[4-(1,3,4-thiadiazol-2-yl)phenyl]methyl}pyrrolidine-1-carboxylate (55 mg, 76.7% yield) as a colorless oil. MS: m/z: Calc'd for C₂₅H₃₃N₃O₇S [M+H]⁺520. found 520.

Step 2: The compound was prepared in 79.1% yield as a colorless oil according to Hydrolysis reaction with LiOH; General Procedure V. MS: m/z: Calc'd for C₂₃H₃₁N₃O₆S [M+H]⁺478. found 478.

Step 3: To the solution of 4-nitrophenyl carbonochloridate (34 mg, 0.17 mmol, 2 equiv.) and tert-butyl (2R,3S,4S)-4-[(tert-butoxycarbonyl)oxy]-3-hydroxy-2-{[4-(1,3,4-thiadiazol-2-yl)phenyl]methyl}pyrrolidine-1-carboxylate (40 mg, 0.084 mmol, 1 equiv.) in DCM (3 mL) was added pyridine (13 mg, 0.17 mmol, 2 equiv.). The mixture was stirred at room temperature for 1 h. The mixture was concentrated. The residue was purified by reversed-phase flash chromatography to afford tert-butyl (2R,3S,4S)-4-[(tert-butoxycarbonyl)oxy]-3-[(4-nitrophenoxycarbonyl)oxy]-2-{[4-(1,3,4-thiadiazol-2-yl)phenyl]methyl}pyrrolidine-1-carboxylate (37 mg, 68.7% yield) as a colorless oil. MS: m/z: Calc'd for C₃₀H₃₄N₄O₁₀S [M+Na]⁺ 643. found 643.

Step 4 and 5: The title compound was prepared in 39.1% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3,4-thiadiazol-2-yl)phenyl]methyl}pyrrolidine-1-carboxylate (129-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 4; Boc Deprotection; General Procedure II in STEP 5. MS: m/z: Calc'd for C₂₁H₂₁FN₄O₃S [M+H]⁺429. found 429. ¹H NMR (400 MHz, Methanol-d₄) δ 9.48 (s, 1H), 8.03-7.96 (m, 2H), 7.50 (d, J=8.2 Hz, 2H), 7.44-7.30 (m, 1H), 7.17 (d, J=7.6 Hz, 1H), 7.14-7.07 (m, 1H), 7.08-7.00 (m, 1H), 5.05-4.96 (m, 1H), 4.46-4.36 (m, 2H), 4.36-4.26 (m, 2H), 3.62 (dd, J=12.6, 4.3 Hz, 1H), 3.32-3.22 (m, 2H), 3.21-3.11 (m, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 9% B to 39% B in 10 min; Wave Length: 254 nm/220 nm; RT1(min): 8.5.

### Synthetic Procedure of (2R,3S,4S)-4-hydroxy-2-{[4′-(trifluoromethyl)-[1,1′-biphenyl]-4-yl]methyl}pyrrolidin-3-yl N-{2-[(2S)-pyrrolidin-2-yl]ethyl}carbamate (V191963)

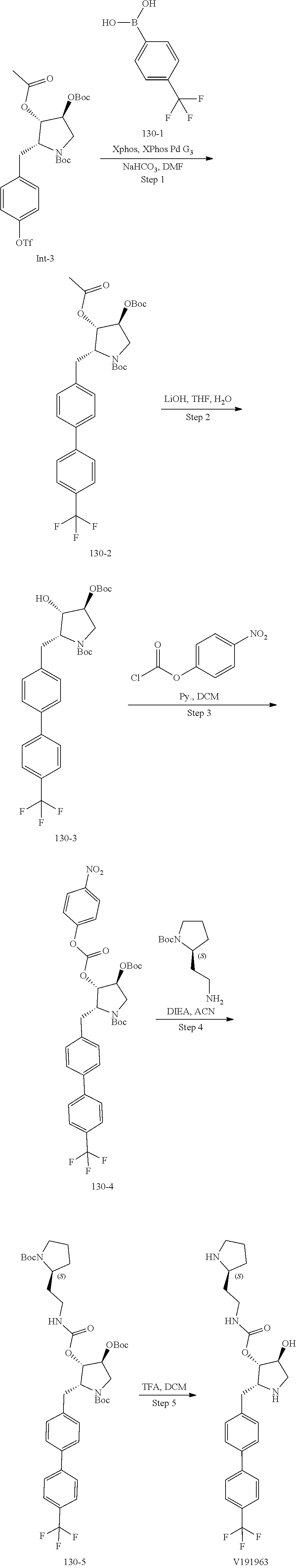

Step 1: To a stirred solution of tert-butyl (2R,3S,4S)-3-(acetyloxy)-4-[(tert-butoxycarbonyl)oxy]-2-{[4-(trifluoromethanesulfonyloxy)phenyl]methyl}pyrrolidine-1-carboxylate (200 mg, 0.34 mmol, 1 equiv) and 4-(trifluoromethyl)phenylboronic acid (78.1 mg, 0.41 mmol, 1.2 equiv) in DMF (5 mL) was added XPhos (16.3 mg, 0.03 mmol, 0.1 equiv), XPhos Pd G₃ (32.7 mg, 0.07 mmol, 0.2 equiv) and NaHCO₃ (57.6 mg, 0.69 mmol, 2 equiv) in portions at room temperature. The resulting mixture was stirred 3 h at 80° C. under a nitrogen atmosphere. Desired product could be detected by LCMS. The residue was purified by reversed-phase flash chromatography to afford tert-butyl (2R,3S,4S)-3-(acetyloxy)-4-[(tert-butoxycarbonyl)oxy]-2-{[4′-(trifluoromethyl)-[1,1′-biphenyl]-4-yl]methyl}pyrrolidine-1-carboxylate (170 mg, 85.6%) as a light yellow oil. MS: m/z: Calc'd for C₃₀H₃₆F₃NO₇ [M+H−56−56]⁺ 468. found, 468.

Step 2: The compound was prepared in 51.3% yield as a light yellow oil according to Hydrolysis reaction with LiOH; General Procedure V. MS: m/z: Calc'd for C₂₈H₃₄F₃NO₆ [M+H]⁺426. found 426.

Step 3: Tert-butyl (2R,3S,4S)-4-((tert-butoxycarbonyl)oxy)-3-(((4-nitrophenoxy)carbonyl)oxy)-2-((4′-(trifluoromethyl)-[1,1′-biphenyl]-4-yl)methyl)pyrrolidine-1-carboxylate. The procedure for tert-butyl (2R,3S,4S)-4-[(tert-butoxycarbonyl)oxy]-3-[(4-nitrophenoxycarbonyl)oxy]-2-{[4-(1,3,4-thiadiazol-2-yl)phenyl]methyl}pyrrolidine-1-carboxylate was repeated with tert-butyl (2R,3S,4S)-4-((tert-butoxycarbonyl)oxy)-3-hydroxy-2-((4′-(trifluoromethyl)-[1,1′-biphenyl]-4-yl)methyl)pyrrolidine-1-carboxylate to afford the title compound. MS: m/z Calc'd for C₂₈H₃₄F₃NO₆ [M+Na]+ 702. found 702.

Step 4 and 5: The title compound was prepared in 26.7% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4′-(trifluoromethyl)-[1,1′-biphenyl]-4-yl]methyl}pyrrolidine-1-carboxylate (130-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate STEP 4; Boc Deprotection; General Procedure II in STEP 5. MS: m/z: Calc'd for C₂₅H₃₀F₃N₃O₃[M+H]⁺478. found 478. ¹H NMR (400 MHz, Methanol-d₄) δ 7.83 (d, J=8.2 Hz, 2H), 7.79-7.75 (m, 2H), 7.74-7.70 (m, 2H), 7.48 (d, J=8.0 Hz, 2H), 5.02 (s, 1H), 4.41 (d, J=4.1 Hz, 1H), 4.27 (d, J=7.3 Hz, 1H), 3.65-3.54 (m, 2H), 3.32-3.21 (m, 6H), 3.13 (dd, J=14.6, 8.3 Hz, 1H), 2.35-2.23 (m, 1H), 2.10 (t, J=7.3 Hz, 1H), 2.05-1.89 (m, 6H), 1.78-1.63 (m, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 13% B to 43% B in 10 min; Wave Length: 254 nm/220 nm; RT1(min): 7.5.

### Synthetic Procedure of (2R,3S,4S)-4-hydroxy-2-{[4′-(trifluoromethyl)-[1,1′-biphenyl]-4-yl]methyl}-pyrrolidin-3-yl N-{2-[(2R)-pyrrolidin-2-yl]ethyl}carbamate; Trifluoroacetic Acid (V192038)

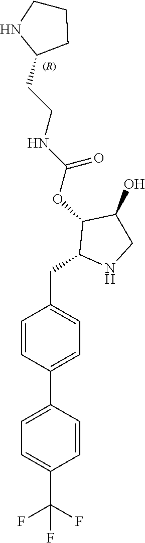

The title compound was prepared in 38.9% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4′-(trifluoromethyl)-[1,1′-biphenyl]-4-yl]methyl}pyrrolidine-1-carboxylate (130-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2R)-2-(2-aminoethyl)pyrrolidine-1-carboxylate in STEP 1; Boc Deprotection; General Procedure II in STEP 2. MS: m/z: Calc'd for C₂₅H₃₀F₃N₃O₃[M+H]⁺478, found 478. ¹H NMR (400 MHz, Methanol-d₄) δ 7.83 (d, J=8.2 Hz, 2H), 7.76 (d, J=8.3 Hz, 2H), 7.72 (d, J=8.0 Hz, 2H), 7.47 (d, J=8.0 Hz, 2H), 5.03 (s, 1H), 4.42 (d, J=4.1 Hz, 1H), 4.29 (d, J=9.0 Hz, 1H), 3.66-3.53 (m, 2H), 3.33-3.22 (m, 3H), 3.32-3.26 (m, 3H), 3.24 (t, J=3.6 Hz, 1H), 3.18-3.08 (m, 1H), 2.37-2.24 (m, 1H), 2.15-1.88 (m, 4H), 1.79-1.64 (m, 1H).

Prep-HPLC-conditions: Column: XBridge Prep Shield RP18 OBD C18 Column, 30*150 mm, 5 μm; Mobile Phase A: Water (0.05% TFA), Mobile Phase B: ACN; Flow rate: 60 mL/min; Gradient: 7% B to 37% B in 10 min; Wave Length: 254 nm/220 nm; RT1(min): 8.5.

### Synthetic Procedure of (2R,3S,4S)-4-hydroxy-2-{[4′-(trifluoromethyl)-[1,1′-biphenyl]-4-yl]methyl}pyrrolidin-3-yl N-[2-(piperidin-4-yl)ethyl]carbamate (V192052)

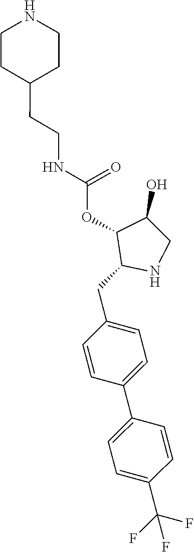 [SMILES: O=C(NCCC1CCNCC1)O[C@@H]1[C@@H](O)CN[C@@H]1Cc1ccc(-c2ccc(C(F)(F)F)cc2)cc1]

The title compound was prepared in 38.9% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4′-(trifluoromethyl)-[1,1′-biphenyl]-4-yl]methyl}pyrrolidine-1-carboxylate (130-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 4-(2-aminoethyl)piperidine-1-carboxylate in STEP 1; Boc Deprotection; General Procedure II in STEP 2. MS: m/z: Calc'd for C₂₆H₃₂F₃N₃O₃[M+H]⁺492, found 492. ¹H NMR (400 MHz, Methanol-d₄) δ 7.82 (d, J=8.2 Hz, 2H), 7.76 (d, J=8.3 Hz, 2H), 7.71 (d, J=7.9 Hz, 2H), 7.47 (d, J=8.0 Hz, 2H), 5.03-4.98 (m, 1H), 4.40 (d, J=4.3 Hz, 1H), 4.27 (s, 1H), 3.61 (dd, J=12.6, 4.4 Hz, 1H), 3.40 (d, J=12.9 Hz, 2H), 3.30-3.21 (m, 4H), 3.12 (dd, J=14.3, 8.7 Hz, 1H), 2.97 (t, J=12.7 Hz, 2H), 2.01 (d, J=14.2 Hz, 2H), 1.72 (s, 1H), 1.62-1.52 (m, 2H), 1.45 (dd, J=18.9, 8.5 Hz, 2H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1 (min): 9.2.

### Synthetic Procedure of (2R,3S,4S)-4-hydroxy-2-{[4′-(trifluoromethyl)-[1,1′-biphenyl]-4-yl]methyl}pyrrolidin-3-yl N-(1,2,3,4-tetrahydroisoquinolin-6-ylmethyl)carbamate (V192058)

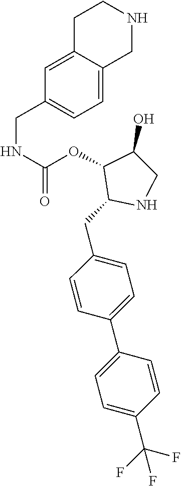 [SMILES: O=C(NCc1ccc2c(c1)CCNC2)O[C@@H]1[C@@H](O)CN[C@@H]1Cc1ccc(-c2ccc(C(F)(F)F)cc2)cc1]

The title compound was prepared in 25.8% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4′-(trifluoromethyl)-[1,1′-biphenyl]-4-yl]methyl}pyrrolidine-1-carboxylate (130-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 6-(aminomethyl)-3,4-dihydro-1H-isoquinoline-2-carboxylate in STEP 1; Boc Deprotection; General Procedure II in STEP 2. MS: m/z: Calc'd for C₂₉H₃₀F₃N₃O₃[M+H]⁺526, found 526. ¹H NMR (400 MHz, Methanol-d₄) δ 7.83 (d, J=8.3 Hz, 2H), 7.77 (d, J=8.3 Hz, 2H), 7.73-7.66 (m, 2H), 7.46 (d, J=8.0 Hz, 2H), 7.28 (d, J=8.5 Hz, 1H), 7.25-7.20 (m, 2H), 5.02 (d, J=3.6 Hz, 1H), 4.45-4.40 (m, 1H), 4.39-4.31 (m, 3H), 4.31-4.24 (m, 1H), 3.62 (dd, J=12.6, 4.3 Hz, 1H), 3.49 (t, J=6.4 Hz, 2H), 3.25 (dd, J=13.5, 7.3 Hz, 3H), 3.17-3.07 (m, 3H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 9.2.

### Synthetic Procedure of (2R,3S,4S)-4-hydroxy-2-((4′-(trifluoromethyl)-[1,1′-biphenyl]-4-yl)methyl)pyrrolidin-3-yl ((2-(azetidin-1-yl)pyridin-4-yl)methyl)carbamate (V192071)

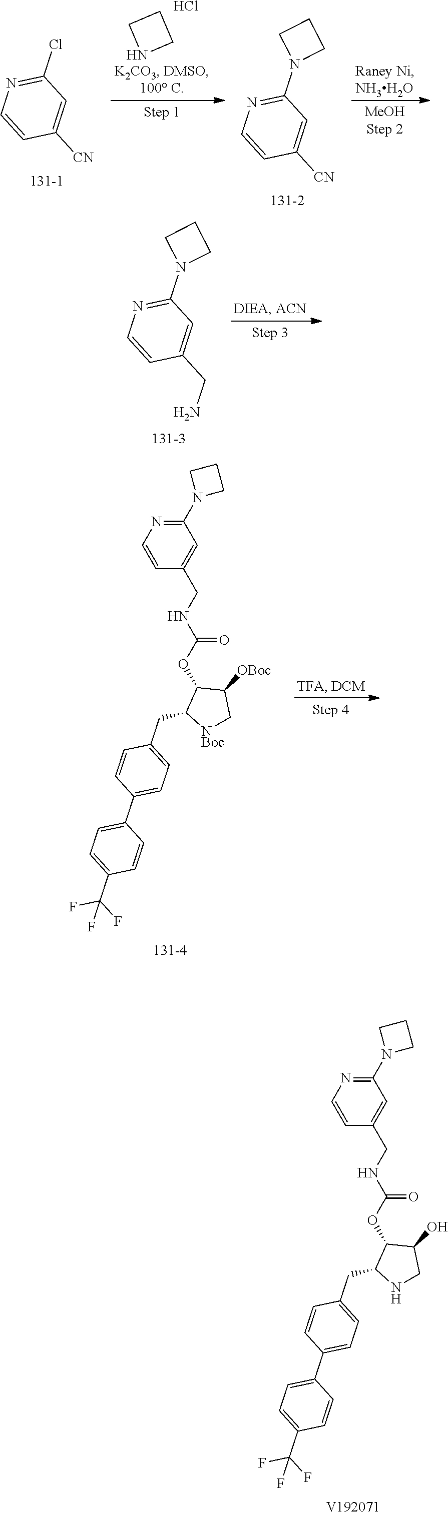

Step 1: To a stirred solution of 2-chloropyridine-4-carbonitrile (300 mg, 2.17 mmol, 1 equiv) and azetidine hydrochloride (405.1 mg, 4.33 mmol, 2 equiv) in DMSO (10 mL) was added K₂CO₃ (897.8 mg, 6.49 mmol, 3 equiv) at room temperature. The resulting mixture was stirred at 100° C. for 12 h under nitrogen atmosphere. The mixture was filtered and the filtrate was purified by flash to afford 2-(azetidin-1-yl)pyridine-4-carbonitrile (246 mg, 71.3%) as a light yellow oil. MS: m/z: Calc'd for C₉H₉N₃ [M+H]⁺160. found 160.

Step 2: To a stirred solution of 2-(azetidin-1-yl)pyridine-4-carbonitrile (230 mg, 1.45 mmol, 1 equiv) and NH₃·H₂O (101.3 mg, 2.89 mmol, 2 equiv) in MeOH (10 mL) was added Raney Ni (804.6 mg, 9.39 mmol, 6.5 equiv) at room temperature under nitrogen atmosphere. The resulting mixture was stirred at room temperature for 2 h under hydrogen atmosphere. After completion of reaction monitored by LCMS. The precipitated solids were collected by filtration and washed with MeOH (3×10 mL). The crude product was used in the next step directly without further purification. MS: m/z: Calc'd for C₉H₁₃N₃[M+H]⁺164. found 164.

Step 3 and 4: The title compound was prepared in 28.8% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4′-(trifluoromethyl)-[1,1′-biphenyl]-4-yl]methyl}pyrrolidine-1-carboxylate (130-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-[2-(azetidin-1-yl)pyridin-4-yl]methanamine STEP 3; Boc Deprotection; General Procedure II in STEP 4. MS: m/z: Calc'd for C₂₈H₂₉F₃N₄O₃[M+H]⁺527. found 527. ¹H NMR (400 MHz, Methanol-d₄) δ 7.97 (d, J=5.4 Hz, 1H), 7.81 (d, J=8.3 Hz, 2H), 7.75 (d, J=8.3 Hz, 2H), 7.58 (d, J=7.9 Hz, 2H), 7.38-7.15 (m, 2H), 6.71-6.61 (m, 1H), 6.35 (s, 1H), 4.70 (d, J=3.8 Hz, 1H), 4.32 (d, J=16.1 Hz, 1H), 4.25-4.16 (m, 2H), 4.00 (t, J=7.4 Hz, 4H), 3.70-3.60 (m, 1H), 3.42-3.36 (m, 1H), 3.02-2.88 (m, 2H), 2.82-2.67 (m, 1H), 2.40-2.29 (m, 2H).

Prep-HPLC purification conditions: Column: XBridge Prep OBD C18 Column, 30*150 mm, 5 μm; Mobile Phase A: Water (10 nmol/LNH₄HCO₃), Mobile Phase B: ACN; Flow rate: 60 mL/min; Gradient: 29% B to 59% B in 10 min; Wave Length: 254 nm/220 nm; RT1 (min): 8.2.

### Synthetic Procedure of (2R,3S,4S)-4-hydroxy-2-{[4′-(trifluoromethyl)-[1,1′-biphenyl]-4-yl]methyl}pyrrolidin-3-yl N-[(3-fluorophenyl)methyl]carbamate (V192083)

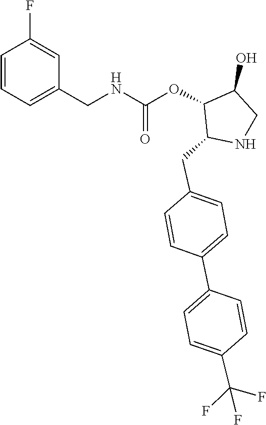 [SMILES: O=C(NCc1cccc(F)c1)O[C@@H]1[C@@H](O)CN[C@@H]1Cc1ccc(-c2ccc(C(F)(F)F)cc2)cc1]

The title compound was prepared in 27.3% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4′-(trifluoromethyl)-[1,1′-biphenyl]-4-yl]methyl}pyrrolidine-1-carboxylate (130-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z: Calc'd for C₂₆H₂₄F₄N₂O₃[M+H]⁺489. found 489. ¹H NMR (400 MHz, Methanol-d₄) δ 7.82 (d, J=8.3 Hz, 2H), 7.76 (d, J=8.4 Hz, 2H), 7.67 (d, J=7.8 Hz, 2H), 7.44 (d, J=8.0 Hz, 2H), 7.41-7.33 (m, 1H), 7.31-7.15 (m, 1H), 7.15-7.08 (m, 1H), 7.08-6.98 (m, 1H), 5.05-4.96 (m, 1H), 4.43 (t, J=2.5 Hz, 1H), 4.37 (d, J=20.0 Hz, 1H), 4.32-4.23 (m, 1H), 3.67-3.58 (m, 1H), 3.41 (s, 1H), 3.25 (dd, J=15.1, 9.9 Hz, 2H), 3.20-3.08 (m, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Wave Length: 254 nm/220 nm; RT1(min): 8.5.

### Synthetic Procedure of (2R,3S,4S)-2-{[4-(5-chlorothiophen-2-yl)phenyl]methyl}-4-hydroxypyrrolidin-3-yl N-[(3-fluorophenyl)methyl]carbamate (V192156)

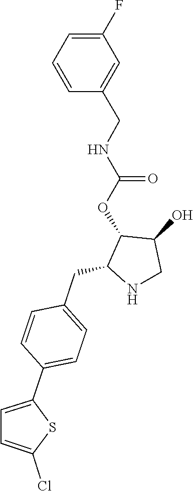 [SMILES: O=C(NCc1cccc(F)c1)O[C@@H]1[C@@H](O)CN[C@@H]1Cc1ccc(-c2ccc(Cl)s2)cc1]

The title compound was prepared in 27.3% overall yield as a white solid according to Substitution reaction for synthesis of Carbamate; General Procedure I using tert-butyl (2R,3S,4S)-4-[(tert-butoxycarbonyl)oxy]-2-{[4-(5-chlorothiophen-2-yl)phenyl]methyl}-3-[(4-nitrophenoxycarbonyl)oxy]pyrrolidine-1-carboxylate (61-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z: Calc'd for C₂₃H₂₂ClFN₂O₃S [M+H]⁺461. Found, 461. ¹H NMR (400 MHz, Methanol-d₄) δ 7.58-7.51 (m, 2H), 7.44-7.30 (m, 3H), 7.25-6.97 (m, 5H), 5.00-4.94 (m, 1H), 4.45-4.20 (m, 4H), 3.60 (dd, J=12.5, 4.3 Hz, 1H), 3.30-3.15 (m, 2H), 3.07 (dd, J=14.0, 8.2 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 27% B to 57% B in 10 min; Wave Length: 254 nm/220 nm; RT1(min): 9.

### Synthetic Procedure of (2R,3S,4S)-4-hydroxy-2-{[4-(1,3-oxazol-5-yl)phenyl]methyl}pyrrolidin-3-yl N-{2-[(4S)-3,3-difluoropiperidin-4-yl]ethyl}carbamate (V192234)

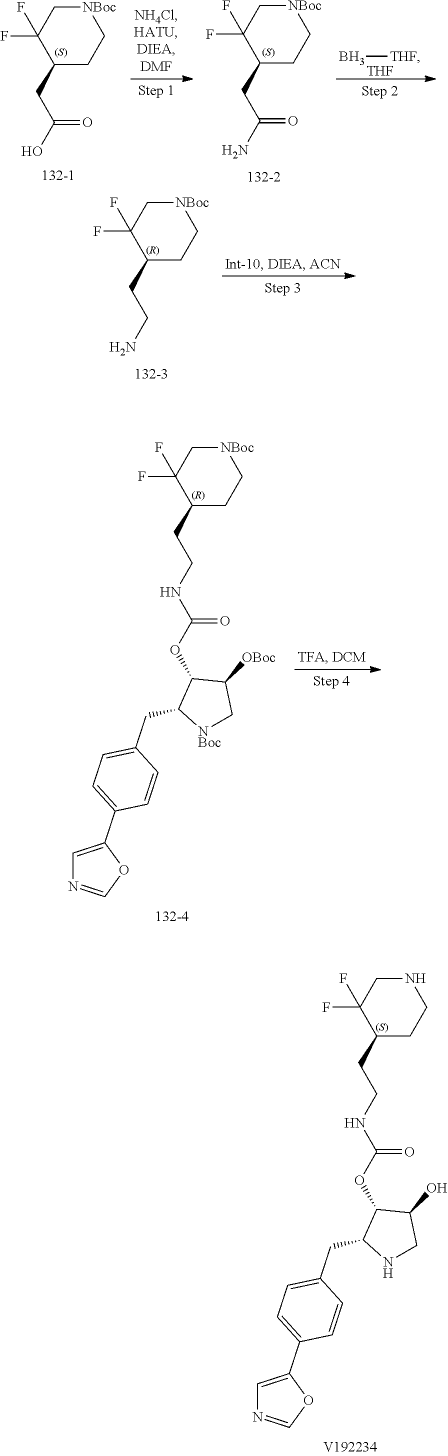

Step 1: To a stirred solution of [(4S)-1-(tert-butoxycarbonyl)-3,3-difluoropiperidin-4-yl]acetic acid (200 mg, 0.72 mmol, 1 equiv) and ammonium chloride (191.5 mg, 3.58 mmol, 5 equiv), ammonium chloride (191.5 mg, 3.58 mmol, 5 equiv) in DMF (5 mL) were added HATU (544.6 mg, 1.43 mmol, 2 equiv) and N,N-Diisopropylethylamine (370.2 mg, 2.86 mmol, 4 equiv) in portions at room temperature. The resulting mixture was stirred over night at 25° C. The mixture was added DMF, filtered and the filtrate was purified by reversed-phase flash chromatography to afford tert-butyl (4S)-4-(carbamoylmethyl)-3,3-difluoropiperidine-1-carboxylate (100 mg) as a white solid. MS: m/z: Calc'd for C₁₂H₂₀F₂N₂O₃[M+H−56]⁺223. Found, 223.

Step 2: To a stirred solution of tert-butyl (4S)-4-(carbamoylmethyl)-3,3-difluoropiperidine-1-carboxylate (80 mg, 0.29 mmol, 1 equiv) in THF (4 mL) was added borane-tetrahydrofuran complex (1.0M in THF) (2 mL) in portions at 0° C. The resulting mixture was stirred 1 h at 25° C. The reaction was quenched by the addition of MeOH (2 mL) at 0° C. The resulting mixture was concentrated under reduced pressure. The resulting mixture was used in the next step directly without further purification. MS: m/z: Calc'd for C₁₂H₂₂F₂N₂O₂[M+H]⁺265. Found, 265.

### Step 3 and 4

The title compound was prepared in 28.8% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (4R)-4-(2-aminoethyl)-3,3-difluoropiperidine-1-carboxylate STEP 3; Boc Deprotection; General Procedure II in STEP 4. MS: m/z: Calc'd for C₂₂H₂₈F₂N₄O₄[M+H]⁺451. Found, 451. ¹H NMR (400 MHz, Methanol-d4) δ 8.27 (s, 1H), 7.76 (d, J=8.1 Hz, 2H), 7.53 (s, 1H), 7.44 (d, J=8.0 Hz, 2H), 5.00 (d, J=3.6 Hz, 1H), 4.41 (d, J=4.0 Hz, 1H), 4.31-4.22 (m, 1H), 3.72 (d, J=8.9 Hz, 1H), 3.60 (dd, J=12.6, 4.3 Hz, 1H), 3.50-3.39 (m, 3H), 3.26-3.08 (m, 5H), 2.25 (d, J=16.1 Hz, 2H), 2.12-2.01 (m, 1H), 1.79-1.67 (m, 1H), 1.63-1.52 (m, 1H).

Prep-HPLC purification conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 22% B in 10 min; Wave Length: 254 nm/220 nm; RT1(min): 8.5.

### Synthetic Procedure of (2R,3S,4S)-2-{[4-(5-chlorothiophen-2-yl)phenyl]methyl}-4-hydroxypyrrolidin-3-yl N-[2-(piperidin-4-yl)ethyl]carbamate (V192261)

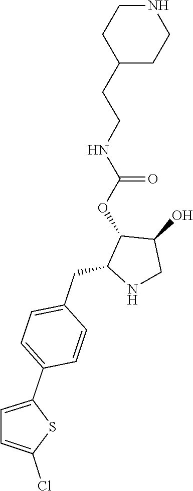 [SMILES: O=C(NCCC1CCNCC1)O[C@@H]1[C@@H](O)CN[C@@H]1Cc1ccc(-c2ccc(Cl)s2)cc1]

The title compound was prepared in 24.6% overall yield as a white solid according to Substitution reaction for synthesis of Carbamate; General Procedure I using tert-butyl (2R,3S,4S)-4-[(tert-butoxycarbonyl)oxy]-2-{[4-(5-chlorothiophen-2-yl)phenyl]methyl}-3-[(4-nitrophenoxycarbonyl)oxy]pyrrolidine-1-carboxylate (61-3) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 4-(2-aminoethyl)piperidine-1-carboxylate in STEP 1; Boc Deprotection; General Procedure II in STEP 2. MS: m/z: Calc'd for C₂₃H₃₀ClN₃O₃S [M+H]⁺464, found 464. ¹H NMR (400 MHz, Methanol-d₄) δ 7.62-7.55 (m, 2H), 7.40-7.34 (m, 2H), 7.23 (d, J=3.9 Hz, 1H), 6.99 (d, J=3.9 Hz, 1H), 5.01-4.95 (m, 1H), 4.42-4.36 (m, 1H), 4.28-4.19 (m, 1H), 3.59 (dd, J=12.7, 4.2 Hz, 1H), 3.44-3.36 (m, 2H), 3.30-3.15 (m, 4H), 3.06 (dd, J=14.1, 8.5 Hz, 1H), 3.02-2.91 (m, 2H), 2.05-1.96 (m, 2H), 1.71 (s, 1H), 1.61-1.51 (m, 2H), 1.50-1.34 (m, 2H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 11% B to 41% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-({4-[1-(methylamino)cyclopropyl]phenyl}methyl)carbamate (V192288)

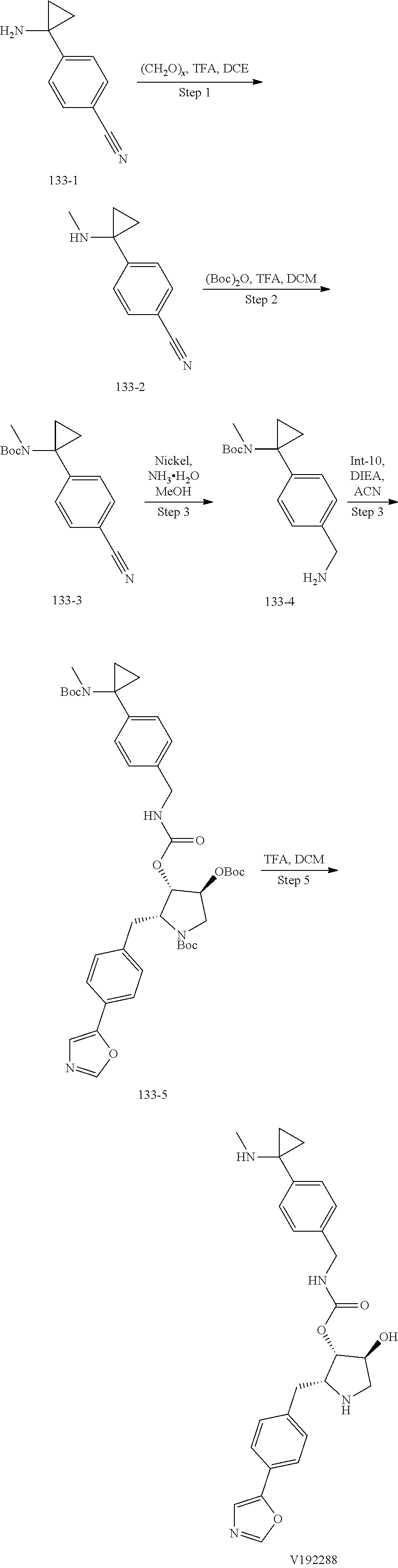

Step 1: To a stirred solution of 4-(1-aminocyclopropyl)benzonitrile (200 mg, 1.26 mmol, 1 equiv) in 1,2-dichloroethane (5 mL) were added paraformaldehyde (167.1 mg, 3.79 mmol, 3 equiv) and trifluoroacetic acid (0.2 mL) at room temperature. The resulting mixture was stirred overnight at 45° C. under a nitrogen atmosphere. The solution was evaporated and the residue was dissolved into DMSO, purified by reversed-phase flash chromatography to afford 4-[1-(methylamino)cyclopropyl]benzonitrile (140 mg) as a yellow solid. MS: m/z: Calc'd for C₁₁H₁₂N₂[M+H]⁺173. Found, 173.

Step 2: To a stirred solution of 4-[1-(methylamino)cyclopropyl]benzonitrile (130 mg, 0.76 mmol, 1 equiv) in 1,4-dioxane (3 mL) and H₂O (1 mL) were added di-tert-butyl dicarbonate (494 mg, 2.27 mmol, 3 equiv) and NaHCO₃ (54 mg, 2.27 mmol, 3 equiv) in portions at room temperature. The resulting mixture was stirred over night at 25° C. under a nitrogen atmosphere. The mixture was concentrated and the residue was dissolved into DMSO, filtered and purified by reversed-phase flash chromatography to afford tert-butyl N-[1-(4-cyanophenyl)cyclopropyl]-N-methylcarbamate (180 mg) as a light yellow solid. MS: m/z: Calc'd for C₁₆H₂₀N₂O₂ [M+H−56]⁺217. Found, 217.

Step 3: To a stirred solution of tert-butyl N-[1-(4-cyanophenyl)cyclopropyl]-N-methylcarbamate (175 mg, 0.64 mmol, 1 equiv) in methanol (5 mL) was added Nickel (113.1 mg, 1.93 mmol, 3 equiv) and ammonium hydroxide (9% in water) (67.6 mg, 1.93 mmol, 3 equiv) dropwise at room temperature. The resulting mixture was stirred over night at 25° C. under H₂ (˜2 bar). Desired The resulting mixture was concentrated under reduced pressure. The residue was used in the next step directly without further purification. MS: m/z: Calc'd for C₁₆H₂₄N₂O₂ [M+H]⁺277. Found, 277.

Step 4 and 5: The title compound was prepared in 28.8% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl N-{1-[4-(aminomethyl)phenyl]cyclopropyl}-N-methylcarbamate (133-4) in STEP 4; Boc Deprotection; General Procedure II in STEP 5. MS: m/z: Calc'd for C₁₇H₂₂N₄O₄ [M+H]⁺347. Found, 347. ¹H NMR (400 MHz, Methanol-d4) δ 8.29 (s, 1H), 7.74 (d, J=7.9 Hz, 2H), 7.61-7.40 (m, 7H), 5.00 (d, J=3.5 Hz, 1H), 4.47-4.33 (m, 3H), 4.27 (s, 1H), 3.61 (d, J=12.0 Hz, 1H), 3.30-3.17 (m, 2H), 3.14-3.08 (m, 1H), 2.60 (s, 3H), 1.42 (d, J=5.9 Hz, 2H), 1.30 (d, J=5.7 Hz, 2H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 2% B to 25% B in 13 min; Wave Length: 254 nm/220 nm; RT1(min): 12.5.

### Synthetic Procedure of (2R,3S,4S)-2-({4-[2-(difluoromethyl)-1,3-thiazol-5-yl]phenyl}methyl)-4-hydroxypyrrolidin-3-yl N-[(3-fluorophenyl)methyl]carbamate (V192310)

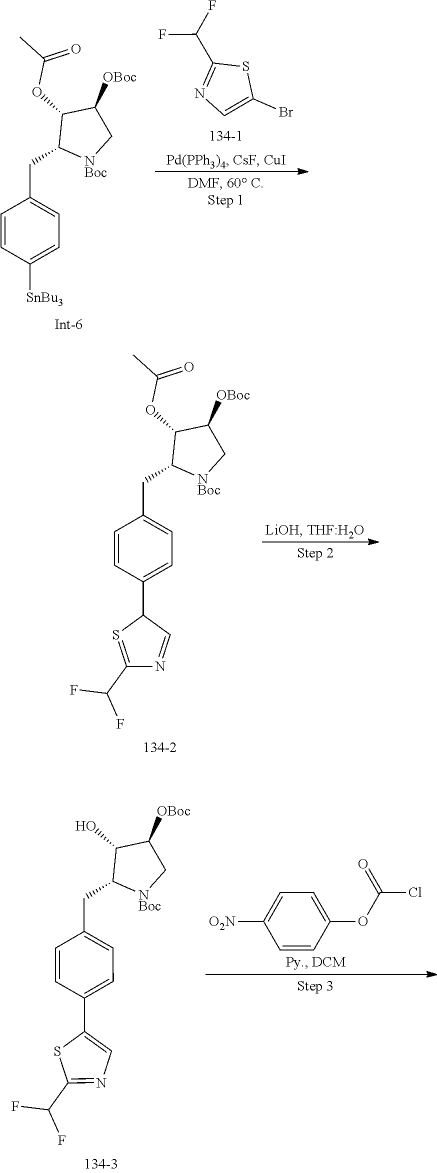

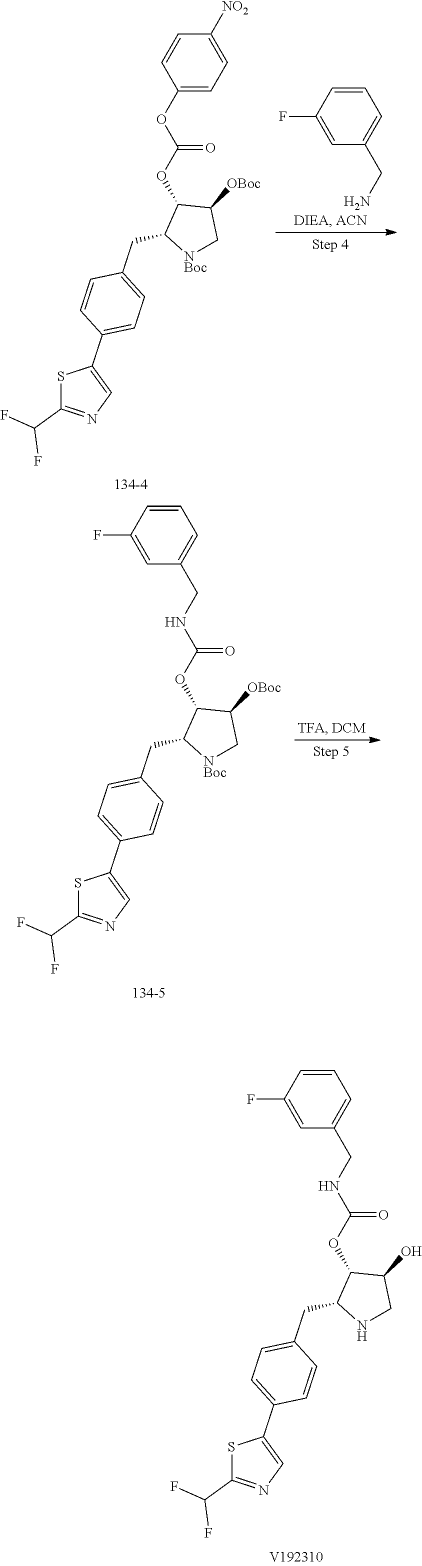

Step 1: To a stirred solution of tert-butyl (2R,3S,4S)-3-(acetyloxy)-4-[(tert-butoxycarbonyl)oxy]-2-{[4-(tributylstannyl)phenyl]methyl}pyrrolidine-1-carboxylate (Int-6, 200 mg, 0.28 mmol, 1 equiv) and 5-bromo-2-(difluoromethyl)-1,3-thiazole (118.2 mg, 0.55 mmol, 2 equiv) in DMF (4 mL) were added Pd₂(dba)₃ (50.6 mg, 0.06 mmol, 0.2 equiv) and tris(furan-2-yl)phosphane (128.2 mg, 0.55 mmol, 2 equiv) at room temperature. The resulting mixture was stirred at 80° C. for 12 h under nitrogen atmosphere. The mixture was added water and extracted with EA. The extracts were washed with brine, dried over anhydrous sodium sulfate, filtered and concentrated. The residue was purified by Prep-TLC (PE:EA, 3:1) to afford tert-butyl (2R,3S,4S)-3-(acetyloxy)-4-[(tert-butoxycarbonyl)oxy]-2-({4-[2-(difluoromethyl)-1,3-thiazol-5-yl]phenyl}methyl)pyrrolidine-1-carboxylate (100 mg, 63.7% yield, 95% purity) as a light yellow oil. MS: m/z: Calc'd for C₂₇H₃₄F₂N₂O₇S [M+H−56−56]⁺457. found 457.

Step 2: The compound was prepared in 68.6% yield as a colorless oil according to Hydrolysis reaction with LiOH; General Procedure V. MS: m/z: Calc'd for C₂₅H₃₂F₂N₂O₆S [M+H+22]⁺549, found 549.

Step 3: To a stirred solution of tert-butyl (2R,3S,4S)-4-[(tert-butoxycarbonyl)oxy]-2-({4-[2-(difluoromethyl)-1,3-thiazol-5-yl]phenyl}methyl)-3-hydroxypyrrolidine-1-carboxylate (100 mg, 0.19 mmol, 1 equiv) and 4-nitrophenyl carbonochloridate (76.6 mg, 0.38 mmol, 2 equiv) in DCM (5 mL) was added pyridine (45.1 mg, 0.57 mmol, 3 equiv) at room temperature. The resulting mixture was stirred at room temperature for 4 h under nitrogen atmosphere. After completion of reaction monitored by LCMS. The residue was purified by Prep-TLC (PE:EA, 2:1). This resulted in tert-butyl (2R,3S,4S)-4-[(tert-butoxycarbonyl)oxy]-2-({4-[2-(difluoromethyl)-1,3-thiazol-5-yl]phenyl}methyl)-3-[(4-nitrophenoxycarbonyl)oxy]pyrrolidine-1-carboxylate (110 mg) as a light yellow oil. MS: m/z: Calc'd for C₃₂H₃₅F₂N₃O₁₀S [M+H+22]⁺714. found 714.

Step 4 and 5: The title compound was prepared in 49.4% overall yield as a white solid according to Substitution reaction for synthesis of Carbamate; General Procedure I using tert-butyl (2R,3S,4S)-4-[(tert-butoxycarbonyl)oxy]-2-({4-[2-(difluoromethyl)-1,3-thiazol-5-yl]phenyl}methyl)-3-[(4-nitrophenoxycarbonyl)oxy]pyrrolidine-1-carboxylate (134-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 4; Boc Deprotection; General Procedure II in STEP 5. MS: m/z: Calc'd for C₂₃H₂₂F₃N₃O₃S [M+H]⁺478. found 478. ¹H NMR (400 MHz, Methanol-d₄) δ 8.08 (s, 1H), 8.00-7.93 (m, 2H), 7.39 (dd, J=13.7, 7.8 Hz, 3H), 7.22-6.96 (m, 4H), 5.00 (s, 1H), 4.47-4.25 (m, 4H), 3.61 (d, J=12.7 Hz, 1H), 3.30-3.19 (m, 2H), 3.11 (t, J=11.5 Hz, 1H).

Prep-HPLC purification conditions: Column: SHIMADZU Shim-pack Scepter C18-120, 30*150 mm, 5 μm; Mobile Phase A: Water (10 nmol/LNH₄HCO₃), Mobile Phase B: ACN; Flow rate: 60 mL/min; Gradient: 2% B to 25% B in 10 min; Wave Length: 254 nm/220 nm; RT1 (min): 11.75.

### Synthetic Procedure of (2R,3S,4S)-4-hydroxy-2-{[4-(1,2,3-thiadiazol-5-yl)phenyl]methyl}pyrrolidin-3-yl N-[(3-fluorophenyl)methyl]carbamate (V192319)

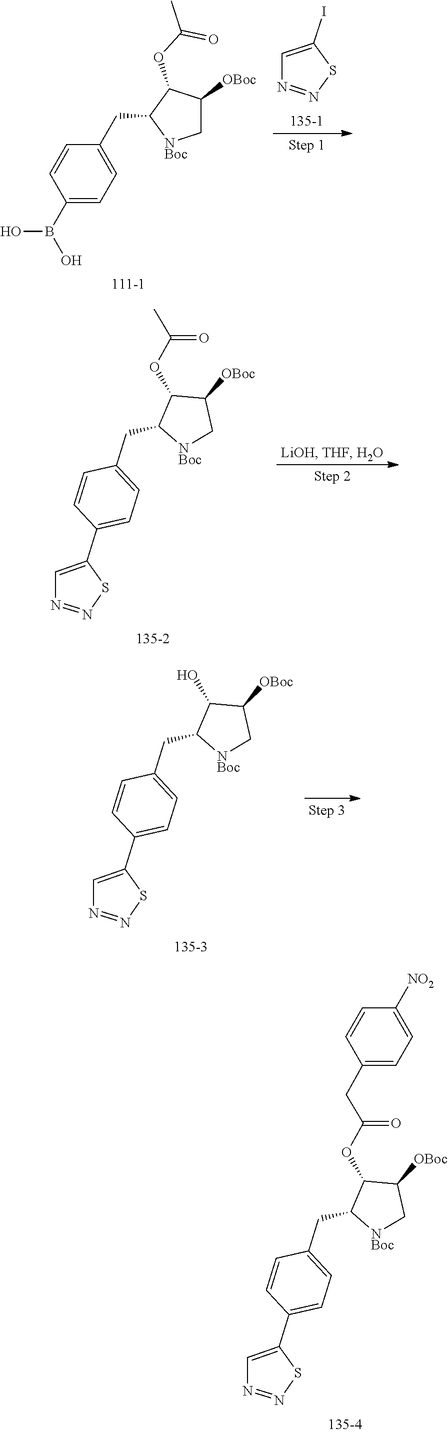

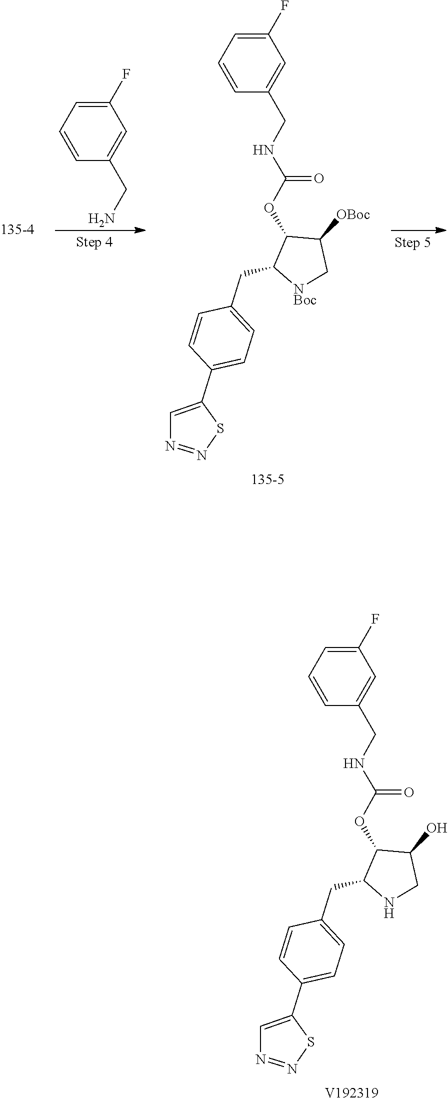

Step 1: To a solution of 5-iodo-1,2,3-thiadiazole (176.92 mg, 0.834 mmol, 2 equiv) and 4-{[(2R,3S,4S)-3-(acetyloxy)-1-(tert-butoxycarbonyl)-4-[(tert-butoxycarbonyl)oxy]pyrrolidin-2-yl]methyl}phenylboronic acid (111-1, 200 mg, 0.417 mmol, 1 equiv.) in 1,4-dioxane (6 mL) and H₂O (0.6 mL) were added Pd(dtbpf)Cl₂ (27.1 mg, 0.04 mmol, 0.1 equiv.) and Na₂CO₃ (88 mg, 0.83 mmol, 2 equiv.). The mixture was stirred at 60° C. overnight under N₂. The mixture was diluted with water and extracted with EA, washed with brine. The organic layer was dried over sodium sulfate, filtered and concentrated. The residue was purified by silica gel column chromatographic to obtain tert-butyl (2R,3S,4S)-3-(acetyloxy)-4-[(tert-butoxycarbonyl)oxy]-2-{[4-(1,2,3-thiadiazol-5-yl)phenyl]methyl}pyrrolidine-1-carboxylate (110 mg, 50.7% yield, 90% purity) as a white solid. MS: m/z: Calc'd for C₂₅H₃₃N₃O₇S [M+H]⁺520. Found, 520.

Step 2: The compound was prepared in 93.2% yield as a colorless oil according to Hydrolysis reaction with LiOH; General Procedure V. MS: m/z: Calc'd for C23H₃₁N₃O₆S [M+H]⁺478. Found, 478.

Step 3: To a solution of tert-butyl (2R,3S,4S)-4-[(tert-butoxycarbonyl)oxy]-3-hydroxy-2-{[4-(1,2,3-thiadiazol-5-yl)phenyl]methyl}pyrrolidine-1-carboxylate (135-3, 110 mg, 0.23 mmol, 1 equiv.) and 4-nitrophenyl carbonochloridate (69.6 mg, 0.34 mmol, 1.5 equiv.) in DCM (5 mL) was added pyridine (36.4 mg, 0.46 mmol, 2 equiv.) at 0° C. The reaction was stirred at room temperature overnight under nitrogen. The mixture was diluted with water and extracted with EA, washed with brine. The organic layer was dried over sodium sulfate, filtered and concentrated. The residue was purified by Prep-TLC (PE/EA, 1:1) to obtain tert-butyl (2R,3S,4S)-4-[(tert-butoxycarbonyl)oxy]-3-[(4-nitrophenoxycarbonyl)oxy]-2-{[4-(1,2,3-thiadiazol-5-yl)phenyl]methyl}pyrrolidine-1-carboxylate (60 mg, 40.5% yield, 90% purity) as a yellow oil. MS: m/z: Calc'd for C₃₀H₃₄N₄O₁₀S [M+H]⁺ 643. Found, 643.

### Step 4 and 5

The title compound was prepared in 49.4% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,2,3-thiadiazol-5-yl)phenyl]methyl}pyrrolidine-1-carboxylate (135-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 4; Boc Deprotection; General Procedure II in STEP 5. MS: m/z: Calc'd for C₂₁H₂₁FN₄O₃S [M+H]⁺429. Found, 429. ¹H NMR (400 MHz, Methanol-d₄) δ 9.12 (s, 1H), 7.74 (d, J=7.9 Hz, 2H), 7.47 (d, J=8.0 Hz, 2H), 7.43-7.34 (m, 1H), 7.17 (d, J=7.7 Hz, 1H), 7.15-7.00 (m, 2H), 4.97 (d, J=3.6 Hz, 1H), 4.48-4.36 (m, 2H), 4.36-4.25 (m, 2H), 3.62 (dd, J=12.6, 4.2 Hz, 1H), 3.25 (dd, J=18.0, 10.4 Hz, 2H), 3.15 (dd, J=13.9, 8.1 Hz, 1H).

Prep-HPLC purification conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 8.5.

### Synthetic Procedure of (2R,3S,4S)-4-hydroxy-2-{[4-(1,2,3-thiadiazol-5-yl)phenyl]methyl}pyrrolidin-3-yl N-{2-[(2S)-pyrrolidin-2-yl]ethyl}carbamate (V192320)

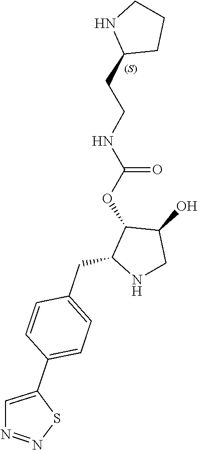

The title compound was prepared in 24.6% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,2,3-thiadiazol-5-yl)phenyl]methyl}pyrrolidine-1-carboxyl-ate (135-4) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate in STEP 1; Boc Deprotection; General Procedure II in STEP 2. MS: m/z: Calc'd for C₂₀H₂₇N₅O₃S [M+H]⁺418 Found, 418. ¹H NMR (400 MHz, Methanol-d₄) δ 9.15 (s, 1H), 7.84-7.77 (m, 2H), 7.55-7.48 (m, 2H), 5.05-4.99 (m, 1H), 4.44-4.39 (m, 1H), 4.34-4.23 (m, 1H), 3.65-3.50 (m, 2H), 3.33-3.21 (m, 6H), 3.20-3.10 (m, 1H), 2.37-2.22 (m, 1H), 2.20-1.85 (m, 4H), 1.80-1.62 (m, 1H).

Prep-HPLC-conditions: Column: XBridge Prep Shield RP18 OBD C18 Column, 30*150 mm, 5 μm; Mobile Phase A: Water (0.05% TFA), Mobile Phase B: ACN; Flow rate: 60 mL/min; Gradient: 2% B to 21% B in 10 min; Wave Length: 254 nm/220 nm; RT1 (min): 8.5.

### Synthetic Procedure of (2R,3S,4S)-2-(2,1,3-benzoxadiazol-5-ylmethyl)-4-hydroxypyrrolidin-3-ylN-[(3-fluorophenyl)methyl]carbamate (V192383)

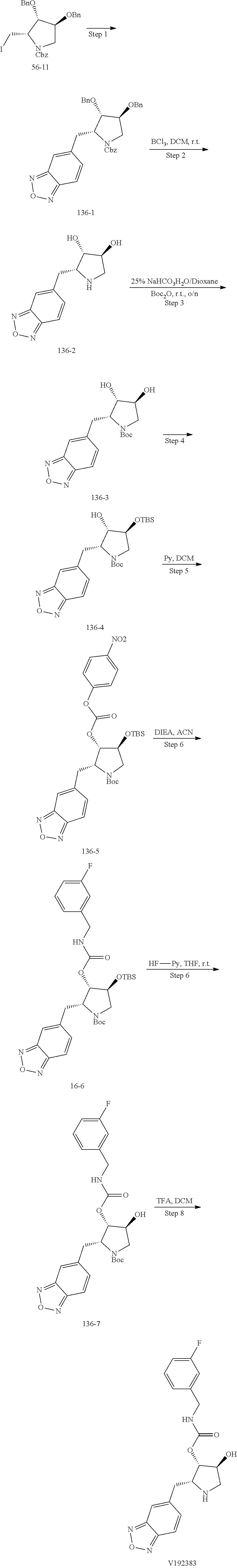

Step 1: To a stirred mixture of benzyl (2S,3S,4S)-3,4-bis(benzyloxy)-2-(iodomethyl)pyrrolidine-1-carboxylate (1 g, 1.79 mmol, 1.00 equiv.) and zinc (1172.9 mg, 17.94 mmol, 10 equiv.) in DMF (5 mL) was added iodometric solution (4553 mg, 17.94 mmol, 10 equiv.) at room temperature under nitrogen atmosphere. The resulting mixture was stirred at room temperature for 1 h under nitrogen atmosphere. The resulting mixture was take the supernatant. To a stirred mixture of 5-bromo-2,1,3-benzoxadiazole (714.0 mg, 3.58 mmol, 2 equiv.) and Pd₂(dba)₃ (328.5 mg, 0.35 mmol, 0.2 equiv.) and Q-Phos (255.0 mg, 0.35 mmol, 0.2 equiv.) in THF (5 mL) was added supernatant at room temperature under nitrogen atmosphere. The above mixture was stirred at room temperature overnight under nitrogen atmosphere. The resulting mixture was concentrated under reduced pressure. The residue was purified by reversed-phase flash chromatography to afford benzyl (2R,3S,4S)-2-(2,1,3-benzoxadiazol-5-ylmethyl)-3,4-bis(benzyloxy)pyrrolidine-1-carboxylate (200 mg, 20.3% yield, 90% purity) as a yellow oil. MS: m/z: Calc'd for C₃₃H₃₁N₃O₅ [M+2]⁺ 550. found 550.

Step 2: To a stirred mixture of benzyl (2R,3S,4S)-2-(2,1,3-benzoxadiazol-5-ylmethyl)-3,4-bis(benzyloxy)pyrrolidine-1-carboxylate (300 mg, 0.54 mmol, 1 equiv.) in DCM (10 mL) was added trichloroborane (639.4 mg, 5.46 mmol, 10 equiv.) at 0° C. The above mixture was stirred at room temperature overnight. The reaction was quenched with MeOH at 0° C. The resulting mixture was concentrated under reduced pressure to afford (2R,3S,4S)-2-(2,1,3-benzoxadiazol-5-ylmethyl)pyrrolidine-3,4-diol (100 mg, 77.8% yield, 90% purity) as a yellow oil. MS: m/z: Calc'd for C₁₁H₁₃N₃O₃ [M+H]⁺ 236. found 236.

Step 3: To a stirred mixture of (2R,3S,4S)-2-(2,1,3-benzoxadiazol-5-ylmethyl)pyrrolidine-3,4-diol (159 mg, 0.67 mmol, 1 equiv) in DCM (5 mL) was added di-tert-butyl dicarbonate (443 mg, 2.02 mmol, 3 equiv.) at room temperature. The above mixture was stirred at room temperature overnight. The resulting mixture was concentrated under reduced pressure. The residue was dissolved in MeCN (5 mL) and purified by reversed-phase flash chromatography to afford tert-butyl (2R,3S,4S)-2-(2,1,3-benzoxadiazol-5-ylmethyl)-3,4-dihydroxypyrrolidine-1-carboxylate (160 mg, 70.9% yield, 90% purity) as a yellow oil. MS: m/z: Calc'd for C₁₆H₂₁N₃O₅ [M+Na]⁺ 358. found 358.

Step 4: To a stirred mixture of tert-butyl (2R,3S,4S)-2-(2,1,3-benzoxadiazol-5-ylmethyl)-3,4-dihydroxypyrrolidine-1-carboxylate (138 mg, 0.41 mmol, 1 equiv.) and imidazole (84.0 mg, 1.23 mmol, 3 equiv.) in DCM (5 mL) was added tert-butyl(chloro)dimethylsilane (68.2 mg, 0.45 mmol, 1.1 equiv.) at room temperature. The above mixture was stirred at room temperature for 3 h. The mixture was concentrated and the residue was purified by Prep-TLC (PE/EA, 3:1) to afford tert-butyl (2R,3S,4S)-2-(2,1,3-benzoxadiazol-5-ylmethyl)-4-[(tert-butyldimethylsilyl)oxy]-3-hydroxypyrrolidine-1-carboxylate (50 mg, 27% yield, 95% purity) as a white solid. MS: m/z: Calc'd for C₂₂H₃₅N₃O₅Si [M−100]⁺ 350. found 350.

Step 5: To a stirred mixture of tert-butyl (2R,3S,4S)-2-(2,1,3-benzoxadiazol-5-ylmethyl)-4-[(tert-butyldimethylsilyl)oxy]-3-hydroxypyrrolidine-1-carboxylate (49 mg, 0.10 mmol, 1 equiv.) and pyridine (25.8 mg, 0.32 mmol, 3 equiv.) in DCM (10 mL) was added 4-nitrophenyl chloroformate (43.93 mg, 0.218 mmol, 2 equiv.) at room temperature. The resulting mixture was stirred at room temperature overnight. The residue was purified by Prep-TLC (PE/EA, 2:1) to afford tert-butyl (2R,3S,4S)-2-(2,1,3-benzoxadiazol-5-ylmethyl)-4-[(tert-butyldimethylsilyl)oxy]-3-[(4-nitrophenoxycarbonyl)oxy]pyrrolidine-1-carboxylate (25 mg, 37.3% yield, 95% purity) as a white solid. MS: m/z: Calc'd for C₂₉H₃₈N₄O₉Si [M−100]⁺515. found 515.

### Step 6, 7, and 8

The title compound was prepared in 91.3% overall yield as an off-white solid according to Substitution reaction for synthesis of Carbamate; General Procedure I using tert-butyl (2R,3S,4S)-2-(2,1,3-benzoxadiazol-5-ylmethyl)-4-[(tert-butyldimethylsilyl)oxy]-3-[(4-nitrophenoxycarbonyl)oxy]pyrrolidine-1-carboxylate (136-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 6; TBS Deprotection; General Procedure VII in STEP 7; Boc Deprotection; General Procedure II in STEP 8. MS: m/z: Calc'd for C₁₉H₁₉FN₄O₄[M+H]⁺387. found 387. ¹H NMR (400 MHz, Methanol-d₄) δ 7.93 (dd, J=9.3, 1.0 Hz, 1H), 7.84 (s, 1H), 7.52 (dd, J=9.3, 1.4 Hz, 1H), 7.47-7.33 (m, 1H), 7.15 (d, J=7.7 Hz, 1H), 7.11-7.06 (m, 1H), 7.05-6.99 (m, 1H), 5.13-5.07 (m, 1H), 4.45 (d, J=4.1 Hz, 1H), 4.42-4.28 (m, 3H), 3.63 (dd, J=12.6, 4.3 Hz, 1H), 3.40-3.34 (m, 1H), 3.33-3.16 (m, 2H).

Prep-HPLC purification conditions: Column: Xselect CSH Prep C18 Column, 30*250 mm, 10 m; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 9.5.

### Synthetic Procedure of (2R,3S,4S)-2-{[4-(2,1,3-benzoxadiazol-5-yl)phenyl]methyl}-4-hydroxypyrrolidin-3-yl N-[(3-fluorophenyl)methyl]carbamate (V192385)

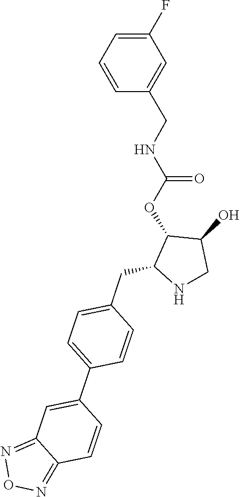 [SMILES: O=C(NCc1cccc(F)c1)O[C@@H]1[C@@H](O)CN[C@@H]1Cc1ccc(-c2ccc3nonc3c2)cc1]

The title compound was prepared in 83.2% overall yield as a white solid according to Substitution reaction for synthesis of Carbamate; General Procedure I using tert-butyl (2R,3S,4S)-2-{[4-(2,1,3-benzoxadiazol-5-yl)phenyl]methyl}-4-[(tert-butoxycarbonyl)oxy]-3-[(4-nitrophenoxycarbonyl)oxy]pyrrolidine-1-carboxylate (136-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z: Calc'd for C₂₅H₂₃FN₄O₄[M+H]⁺463. Found, 463. ¹H NMR (400 MHz, Methanol-d₄) 6.11-8.06 (m, 1H), 8.02 (dd, J=9.4, 1.1 Hz, 1H), 7.91-7.84 (m, 1H), 7.76 (d, J=8.1 Hz, 2H), 7.48 (d, J=8.0 Hz, 2H), 7.41-7.31 (m, 1H), 7.18 (d, J=7.6 Hz, 1H), 7.12 (d, J=9.9 Hz, 1H), 7.06-7.01 (m, 1H), 5.05-4.96 (m, 1H), 4.49-4.25 (m, 4H), 3.63 (dd, J=12.7, 4.3 Hz, 1H), 3.27 (dd, J=13.6, 8.3 Hz, 2H), 3.16 (dd, J=13.8, 8.2 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 23% B to 53% B in 10 min; Wave Length: 254 nm/220 nm; RT1 (min): 8.5.

### Synthetic Procedure of (3S,4S,5R)-4-({[(3-fluorophenyl)methyl]carbamoyl}oxy)-5-{[4-(1,3-oxazol-5-yl)phenyl]methyl}pyrrolidin-3-yl acetate (V192392)

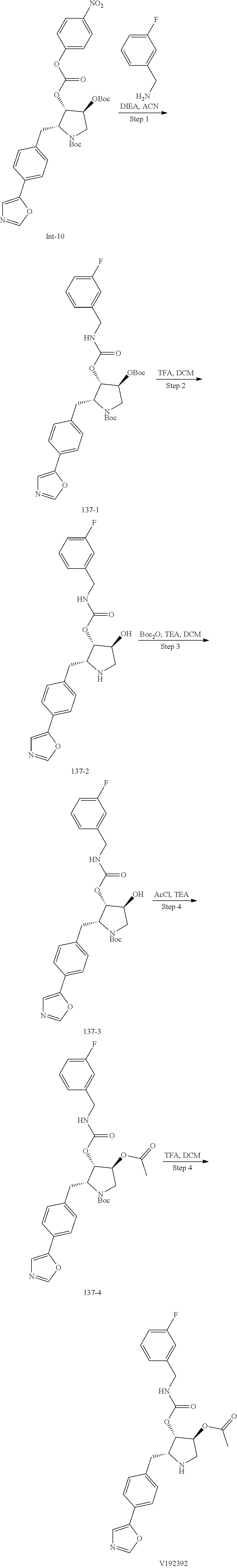

### Step 1 and 2

The compound was prepared in 90.2% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z: Calc'd for C₂₂H₂₂FN₃O₄[M+H]⁺412. found 412.

Step 3: To a stirred solution of (2R,3S,4S)-4-hydroxy-2-{[4-(1,3-oxazol-5-yl)phenyl]methyl}pyrrolidin-3-yl N-[(3-fluorophenyl)methyl]carbamate (100 mg, 0.24 mmol, 1 equiv) and Et₃N (122.9 mg, 1.22 mmol, 5 equiv) in DCM (10 mL) was added di-tert-butyl dicarbonate (159.1 mg, 0.73 mmol, 3 equiv) at 0° C. The resulting mixture was stirred at room temperature for 4 h under nitrogen atmosphere and concentrated. The residue was purified by Prep-TLC (EA) to afford tert-butyl (2R,3S,4S)-3-({[(3-fluorophenyl)methyl]carbamoyl}oxy)-4-hydroxy-2-{[4-(1,3-oxazol-5-yl)phenyl]methyl}pyrrolidine-1-carboxylate (80 mg, 64.3% yield, 98% purity) as a light yellow oil. MS: m/z: Calc'd for C₂₇H₃₀FN₃O₆ [M+H]⁺512. found 512.

Step 4: To a stirred solution of tert-butyl (2R,3S,4S)-3-({[(3-fluorophenyl)methyl]carbamoyl}oxy)-4-hydroxy-2-{[4-(1,3-oxazol-5-yl)phenyl]methyl}pyrroli-dine-1-carboxylate (75 mg, 0.15 mmol, 1 equiv) and Et₃N (44.5 mg, 0.44 mmol, 3 equiv) in DCM (15 mL) was added acetyl chloride (12.7 mg, 0.16 mmol, 1.1 equiv) at 0° C. The mixture was stirred at room temperature for 1 h under nitrogen atmosphere. The resulting mixture was purified by Prep-TLC (PE: EA, 2:1) to afford tert-butyl (2R,3S,4S)-4-(acetyloxy)-3-({[(3-fluorophenyl)methyl]carbamoyl}oxy)-2-{[4-(1,3-oxazol-5-yl)phenyl]methyl}pyrrolidine-1-carboxylate (70 mg, 86.3% yield, 95% purity) as a light yellow solid. MS: m/z: Calc'd for C₂₉H₃₂FN₃O₇[M+H]⁺554. found 554.

Step 5: The title compound was prepared in 26.9% yield as a white solid according to Boc Deprotection; General Procedure II. MS: m/z: Calc'd for C₂₄H₂₄FN₃O₅ [M+H]⁺454. found 454. ¹H NMR (400 MHz, Methanol-d₄) δ 8.40 (s, 1H), 8.26 (s, 1H), 7.68 (d, J=7.9 Hz, 2H), 7.50 (s, 1H), 7.37 (t, J=7.3 Hz, 3H), 7.16 (d, J=7.7 Hz, 1H), 7.10 (d, J=9.9 Hz, 1H), 7.03 (t, J=8.6 Hz, 1H), 5.19 (d, J=5.3 Hz, 1H), 5.03 (d, J=3.9 Hz, 1H), 4.39 (d, J=15.3 Hz, 1H), 4.30 (d, J=15.5 Hz, 1H), 3.87 (d, J=3.9 Hz, 1H), 3.66 (dd, J=13.2, 5.6 Hz, 1H), 3.10 (dd, J=13.9, 6.8 Hz, 2H), 2.99 (dd, J=13.9, 7.9 Hz, 1H), 2.07 (s, 3H).

Prep-HPLC purification conditions: Column: Xselect CSH Prep C18 OBD Column, 30*150 mm, 5 μm; Mobile Phase A: Water (0.1% FA), Mobile Phase B: ACN; Flow rate: 60 mL/min; Gradient: 3% B to 33% B in 10 min; Wave Length: 254 nm/220 nm; RT1(min): 9.07.

### Synthetic Procedure of (3S,4S,5R)-4-({[(3-fluorophenyl)methyl]carbamoyl}oxy)-5-{[4-(1,3-thiazol-5-yl)phenyl]methyl}pyrrolidin-3-yl acetate (V192393)

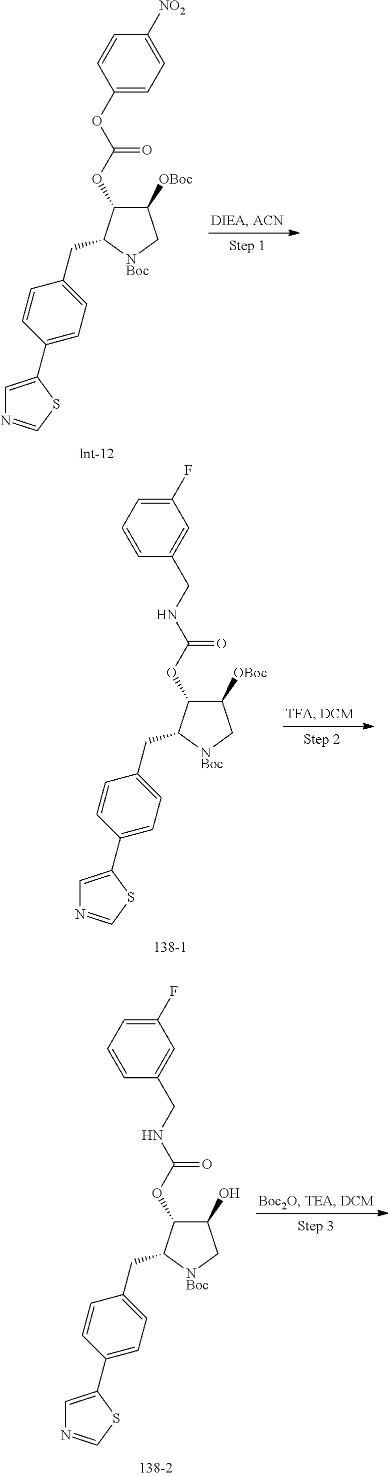

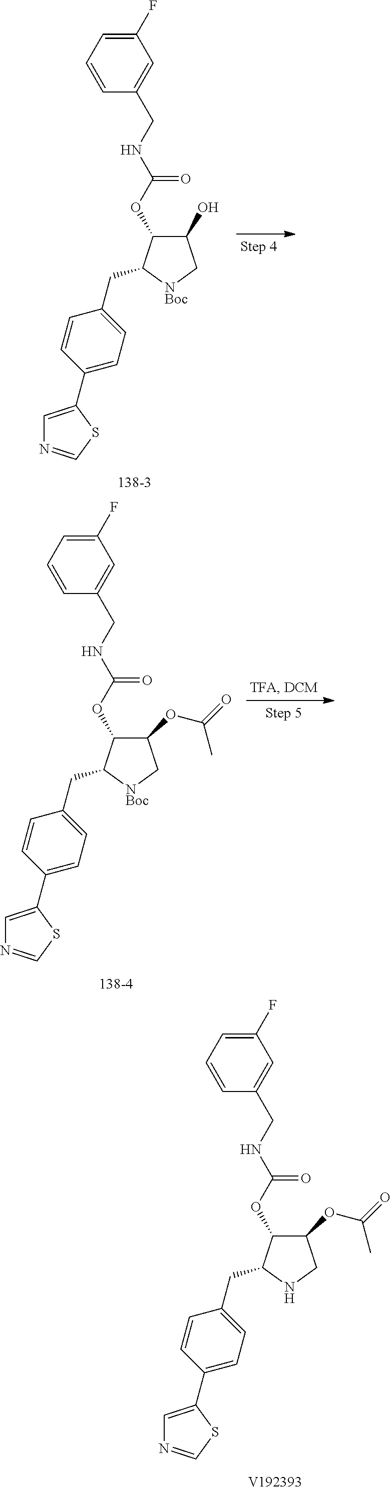 [SMILES: CC(C)(C)OC(=O)N1C[C@H](O)[C@@H](OC(=O)NCc2cccc(F)c2)[C@H]1Cc1ccc(-c2cncs2)cc1 ; CC(=O)O[C@H]1CN(C(=O)OC(C)(C)C)[C@H](Cc2ccc(-c3cncs3)cc2)[C@@H]1OC(=O)NCc1cccc(F)c1 ; CC(=O)O[C@H]1CN[C@H](Cc2ccc(-c3cncs3)cc2)[C@@H]1OC(=O)NCc1cccc(F)c1]

### Step 1 and 2

The compound was prepared in 89.2% overall yield as a yellow oil according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-thiazol-5-yl)phenyl]methyl}pyrrolidine-1-carboxylate (Int-12) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z: Calc'd for C₂₂H₂₂FN₃O₃S [M+H]⁺428. found 428.

Step 3: To a stirred mixture of (2R,3S,4S)-4-hydroxy-2-{[4-(1,3-thiazol-5-yl)phenyl]methyl}pyrrolidin-3-yl N-[(3-fluorophenyl)methyl]carbamate (100 mg, 0.23 mmol, 1 equiv.) and Et₃N (71.01 mg, 0.702 mmol, 3 equiv.) in DCM (5 mL) was added di-tert-butyl dicarbonate (102.11 mg, 0.48 mmol, 2 equiv.) at room temperature. The above mixture was stirred at room temperature overnight. The resulting mixture was concentrated under reduced pressure. The residue was dissolved in MeCN (5 mL) and purified by reversed-phase flash chromatography to afford tert-butyl (2R,3S,4S)-3-({[(3-fluorophenyl)methyl]carbamoyl}oxy)-4-hydroxy-2-{[4-(1,3-thiazol-5-yl)phenyl]methyl}pyrrolidine-1-carboxylate (120 mg, 97.2% yield, 95% purity) as a yellow solid. MS: m/z: Calc'd for C₂₇H₃₀FN₃O₅S [M+H]⁺528. found 528.

Step 4: To a stirred mixture of tert-butyl (2R,3S,4S)-3-({[(3-fluorophenyl)methyl]carbamoyl}oxy)-4-hydroxy-2-{[4-(1,3-thiazol-5-yl)phenyl]methyl}pyrrol-idine-1-carboxylate (120 mg, 0.27 mmol, 1 equiv.) and Et₃N (69.5 mg, 0.68 mmol, 3 equiv.) in DCM (10 mL) was added acetyl chloride (35.71 mg, 0.454 mmol, 2 equiv,) at room temperature. The mixture was stirred at room temperature for 1 h. The resulting mixture was concentrated under reduced pressure. The residue was dissolved in MeCN (5 mL) and purified by reversed-phase flash chromatography to afford tert-butyl (2R,3S,4S)-4-(acetyloxy)-3-({[(3-fluorophenyl)methyl]carbamoyl}oxy)-2-{[4-(1,3-thiazol-5-yl)phenyl]methyl}pyrrolidine-1-carboxylate (125 mg, 98% yield, 95% purity) as a yellow solid. MS: m/z: Calc'd for C₂₉H₃₂FN₃O₆S [M−100]⁺570. found 570.

Step 5: The title compound was prepared in 26.9% yield as a white solid according to Boc Deprotection; General Procedure II. MS: m/z: Calc'd for C₂₄H₂₄FN₃O₄S [M+H]⁺470. found 470. ¹H NMR (400 MHz, Methanol-d4) δ 8.99 (s, 1H), 8.20-8.15 (m, 1H), 7.69-7.61 (m, 2H), 7.39 (dd, J=14.2, 7.9 Hz, 3H), 7.29-7.15 (m, 1H), 7.15-7.09 (m, 1H), 7.08-6.99 (m, 1H), 5.36-5.30 (m, 1H), 5.24-5.18 (m, 1H), 4.46-4.29 (m, 2H), 4.27-4.17 (m, 1H), 3.88-3.79 (m, 1H), 3.43 (d, J=13.7 Hz, 1H), 3.25 (dd, J=14.1, 7.0 Hz, 1H), 3.10 (s, 1H), 2.13-2.08 (m, 3H).

Prep-HPLC purification conditions: Column: Xselect CSH Prep C18 Column, 30*250 mm, 10 m; Mobile Phase A: Water (0.05% TFA), Mobile Phase B: ACN; Flow rate: 60 mL/min; Gradient: 16% B to 46% B in 10 min; Wave Length: 254 nm/220 nm; RT1(min): 8.5.

### Synthetic Procedure of (2R,3S,4S)-4-hydroxy-2-{[4-(1,3-thiazol-5-yloxy)phenyl]methyl}pyrrolidin-3-yl N-[(3-fluorophenyl)methyl]carbamate (V192395)

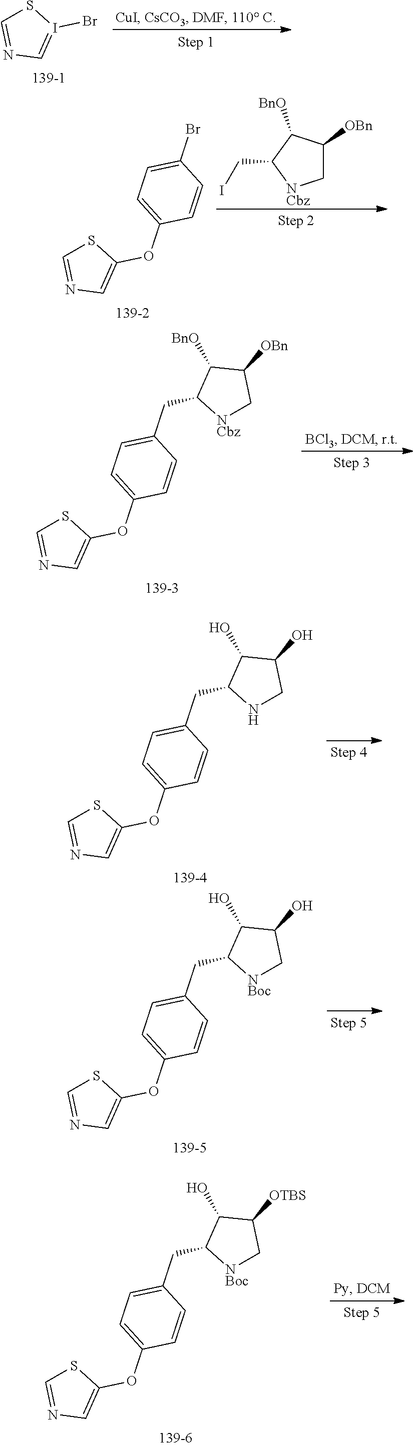

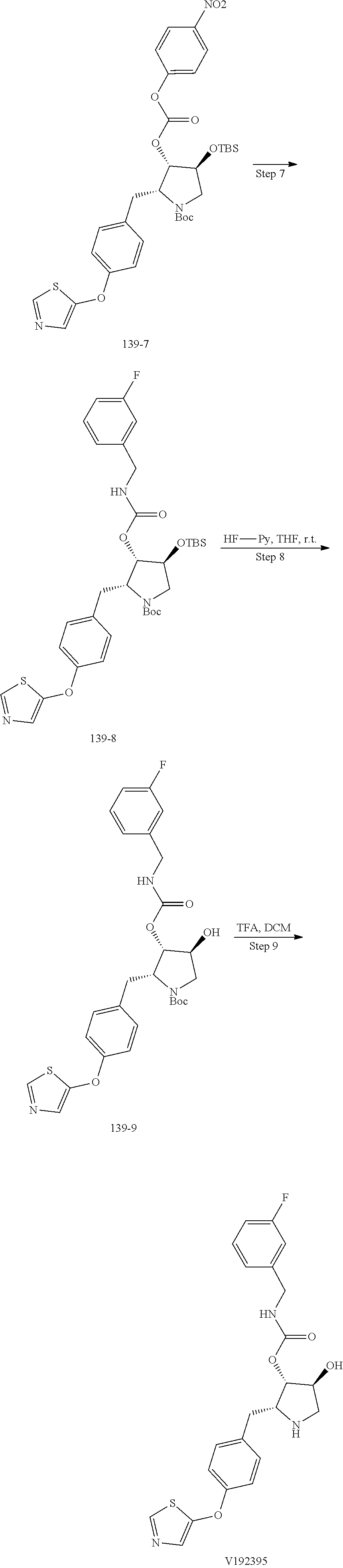

Step 1: To a stirred mixture of 4-bromophenol (8.57 g, 49.53 mmol, 1.2 equiv) and 5-bromo-1,3-thiazole (6.5 g, 39.6 mmol, 1.00 equiv.) in DMF (10 mL) was added CuI (7.6 g, 39.62 mmol, 1 equiv.) and Cs₂CO₃ (38.7 g, 118.8 mmol, 3 equiv.) at room temperature under nitrogen atmosphere. The resulting mixture was stirred at 110° C. overnight under nitrogen atmosphere. The reaction mixture was quenched with water and extracted with EA. The extracts were washed with brine, dried over anhydrous sodium sulfate, filtered and concentrated. The residue was purified by reversed-phase flash chromatography to afford 5-(4-bromophenoxy)-1,3-thiazole (3 g, 29.5% yield, 90% purity) as a white solid. MS: m/z: Calc'd for C₉H₆BrNOS [M+H]⁺ 258. found 258.

Step 2: To a stirred mixture of benzyl (2S,3S,4S)-3,4-bis(benzyloxy)-2-(iodomethyl)pyrrolidine-1-carboxylate (1 g, 1.79 mmol, 1 equiv.) and zinc (1173 mg, 17.94 mmol, 10 equiv.) in DMF (5 mL) were added Iodometric solution (4553 mg, 17.94 mmol, 10 equiv.) at room temperature under nitrogen atmosphere. The resulting mixture was stirred at room temperature for 1 h under nitrogen atmosphere. The resulting mixture was take the supernatant. To a stirred mixture of 5-(4-bromophenoxy)-1,3-thiazole (0.92 g, 3.588 mmol, 2 equiv.), Pd₂(dba)₃ (328 mg, 0.35 mmol, 0.2 equiv.) and Q-Phos (255 mg, 0.35 mmol, 0.2 equiv.) in THF (5 mL) was added supernatant at room temperature under nitrogen atmosphere. The above mixture was stirred at room temperature overnight under nitrogen atmosphere. The resulting mixture was concentrated under reduced pressure. The residue was purified by reversed-phase flash chromatography to afford benzyl (2R,3S,4S)-3,4-bis(benzyloxy)-2-{[4-(1,3-thiazol-5-yloxy)phenyl]methyl}pyrrolidine-1-carboxylate (300 mg, 27.5% yield, 90% purity) as a yellow solid. MS: m/z: Calc'd for C₃₆H₃₄N₂O₅S [M+H]⁺ 607. found 607.

Step 3: To a stirred mixture of benzyl (2R,3S,4S)-3,4-bis(benzyloxy)-2-{[4-(1,3-thiazol-5-yloxy)phenyl]methyl}pyrrolidine-1-carboxylate (470 mg, 0.77 mmol, 1 equiv.) in DCM (10 mL) was added trichloroborane (908 mg, 7.75 mmol, 10 equiv.) at 0° C. The resulting mixture was stirred at room temperature overnight. The reaction was quenched with MeOH at 0° C. The resulting mixture was concentrated under reduced pressure to afford the crude (2R,3S,4S)-2-{[4-(1,3-thiazol-5-yloxy)phenyl]methyl}pyrrolidine-3,4-diol (200 mg, 88.3% yield, 90% purity) as a yellow oil which was used for the next step directly. MS: m/z: Calc'd for C₁₄H₁₆N₂O₃S [M+H]⁺ 293. found 293.

Step 4: To a stirred mixture of (2R,3S,4S)-2-{[4-(1,3-thiazol-5-yloxy)phenyl]methyl}pyrrolidine-3,4-diol (186 mg, 0.63 mmol, 1 equiv.) and di-tert-butyl dicarbonate (278 mg, 1.27 mmol, 2 equiv.) in DCM (5 mL) was added Et₃N (193.1 mg, 1.908 mmol, 3 equiv.) at room temperature. The above mixture was stirred at room temperature overnight. The resulting mixture was concentrated under reduced pressure. The residue was dissolved in MeCN (5 mL) and purified by reversed-phase flash chromatography to afford tert-butyl (2R,3S,4S)-3,4-dihydroxy-2-{[4-(1,3-thiazol-5-yloxy)phenyl]methyl}pyrrolidine-1-carboxylate (190 mg, 76.1% yield, 90% purity) as a yellow solid. MS: m/z: Calc'd for C₁₉H₂₄N₂O₅S [M+Na]⁺ 415. found 415.

Step 5: To a stirred mixture of tert-butyl (2R,3S,4S)-3,4-dihydroxy-2-{[4-(1,3-thiazol-5-yloxy)phenyl]methyl}pyrrolidine-1-carboxylate (165 mg, 0.420 mmol, 1 equiv.) and imidazole (85.8 mg, 1.26 mmol, 3 equiv.) in DCM (5 mL) was added tert-butyl(chloro)dimethylsilane (69.7 mg, 0.46 mmol, 1.1 equiv.) at room temperature. The above mixture was stirred at room temperature for 3 h. The resulting mixture was purified by Prep-TLC (PE/EA, 3:1) to afford tert-butyl (2R,3S,4S)-4-[(tert-butyldimethylsilyl)oxy]-3-hydroxy-2-{[4-(1,3-thiazol-5-yloxy)phenyl]methyl}pyrrolidine-1-carboxylate (100 mg, 47% yield, 95% purity) as a yellow solid. MS: m/z: Calc'd for C₂₅H₃₈N₂O₅SSi [M+H]⁺ 529. found 529.

Step 6: To a stirred mixture of tert-butyl (2R,3S,4S)-4-[(tert-butyldimethylsilyl)oxy]-3-hydroxy-2-{[4-(1,3-thiazol-5-yloxy)phenyl]methyl}pyrrolidine-1-carboxylate (65 mg, 0.13 mmol, 1 equiv.) and pyridine (30.4 mg, 0.38 mmol, 3 equiv.) in DCM (10 mL) was added 4-nitrophenyl chloroformate (51.71 mg, 0.256 mmol, 2 equiv.) at room temperature. The above mixture was stirred at room temperature overnight. The resulting mixture was purified by Prep-TLC (PE/EA 2:1) to afford tert-butyl (2R,3S,4S)-4-[(tert-butyldimethylsilyl)oxy]-3-[(4-nitrophenoxycarbonyl)oxy]-2-{[4-(1,3-thiazol-5-yloxy)phenyl]methyl}pyrrolidine-1-carboxylate (50 mg, 58% yield, 90% purity) as a white solid. MS: m/z: Calc'd for C₃₂H₄₁N₃O₉SSi [M+H]⁺672. found 672.

### Step 7, 8, and 9

The title compound was prepared in 37.5% overall yield as a white solid according to Substitution reaction for synthesis of Carbamate; General Procedure I using tert-butyl (2R,3S,4S)-4-[(tert-butyldimethylsilyl)oxy]-3-[(4-nitrophenoxycarbonyl)oxy]-2-{[4-(1,3-thiazol-5-yloxy)phenyl]methyl}pyrrolidine-1-carboxylate (139-7) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 7; TBS Deprotection; General Procedure VII in STEP 8; Boc Deprotection; General Procedure II in STEP 9. MS: m/z: Calc'd for C₂₂H₂₂FN₃O₄S [M+H]⁺444. found 444. ¹H NMR (400 MHz, Methanol-d₄) δ 8.66 (s, 1H), 7.50 (d, J=0.9 Hz, 1H), 7.44-7.28 (m, 3H), 7.15 (d, J=7.7 Hz, 1H), 7.08 (dd, J=9.2, 2.7 Hz, 3H), 7.09-6.96 (m, 1H), 4.97-4.92 (m, 1H), 4.46-4.28 (m, 3H), 4.28-4.15 (m, 1H), 3.59 (dd, J=12.7, 4.2 Hz, 1H), 3.25 (d, J=12.6 Hz, 1H), 3.17 (dd, J=14.0, 7.7 Hz, 1H), 3.06 (dd, J=14.0, 8.1 Hz, 1H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 14% B to 44% B in 10 min; Wave Length: 254 nm/220 nm; RT1(min): 9.5.

### Synthetic Procedure of (2R,3S,4S)-2-(3-fluoro-4-(oxazol-5-yl)benzyl)-4-hydroxypyrrolidin-3-yl (2-((S)-azetidin-2-yl)ethyl)carbamate (V192991)

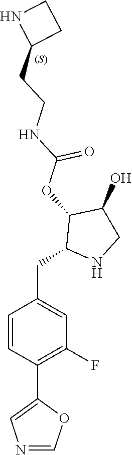

The title compound was prepared in 38.2% overall yield as a white solid according to Substitution reaction for synthesis of Carbamate; General Procedure I using tert-butyl (2R,3S,4S)-4-((tert-butyldimethylsilyl)oxy)-2-(3-fluoro-4-(oxazol-5-yl)benzyl)-3-(((4-nitrophenoxy)carbonyl)oxy)pyrrolidine-1-carboxylate (144-8) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (R)-2-(2-aminoethyl)azetidine-1-carboxylate in STEP 1; TBS Deprotection; General Procedure VII in STEP 2; Boc Deprotection; General Procedure II in STEP 3. MS: m/z: Calc'd for C₂₀H₂₅FN₄O₄[M+H]⁺405. Found, 405. ¹H NMR (400 MHz, Methanol-d₄) δ 8.35 (s, 1H), 7.91-7.82 (m, 1H), 7.53 (d, J=3.7 Hz, 1H), 7.36-7.27 (m, 2H), 5.04-4.98 (m, 1H), 4.59-4.46 (m, 1H), 4.41 (d, J=3.9 Hz, 1H), 4.34-4.25 (m, 1H), 4.07 (q, J=9.3 Hz, 1H), 3.98-3.87 (m, 1H), 3.60 (dd, J=12.5, 4.2 Hz, 1H), 3.31-3.11 (m, 5H), 2.69-2.56 (m, 1H), 2.46-2.32 (m, 1H), 2.25-2.03 (m, 2H), 1.31 (s, 2H).

Prep-HPLC-conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min mL/min; Gradient: 2% B to 32% B in 13 min; Wave Length: 254 nm/220 nm; RT1(min): 4.67.

### Synthetic Procedure of (2R,3S,4S)-2-(2-fluoro-4-(oxazol-5-yl)benzyl)-4-hydroxypyrrolidin-3-yl (2-((S)-azetidin-2-yl)ethyl)carbamate (V192990)

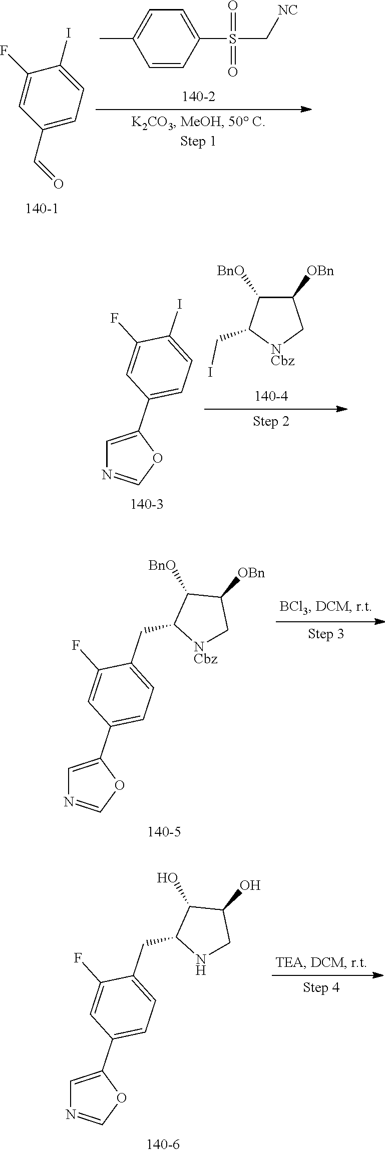

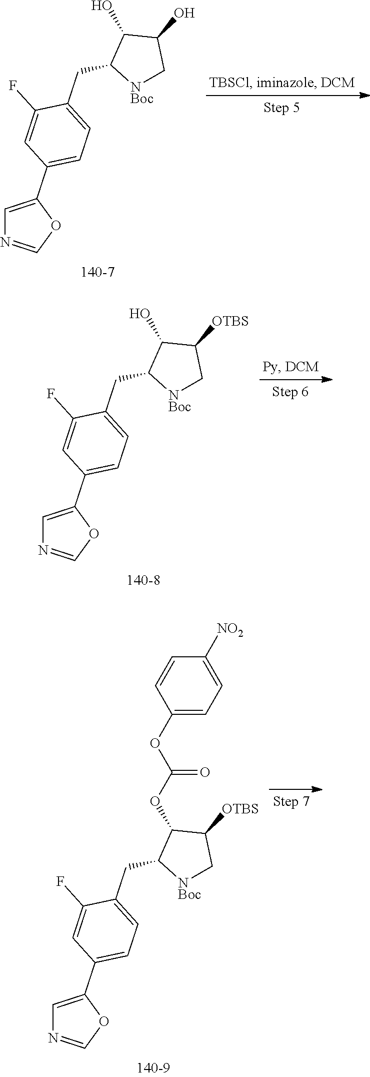 [SMILES: CC(C)(C)OC(=O)N1C[C@H](O)[C@@H](O)[C@H]1Cc1ccc(-c2cnco2)cc1F ; CC(C)(C)OC(=O)N1C[C@H](O[Si](C)(C)C(C)(C)C)[C@@H](O)[C@H]1Cc1ccc(-c2cnco2)cc1F ; CC(C)(C)OC(=O)N1C[C@H](O[Si](C)(C)C(C)(C)C)[C@@H](OC(=O)Oc2ccc([N+](=O)[O-])cc2)[C@H]1Cc1ccc(-c2cnco2)cc1F]

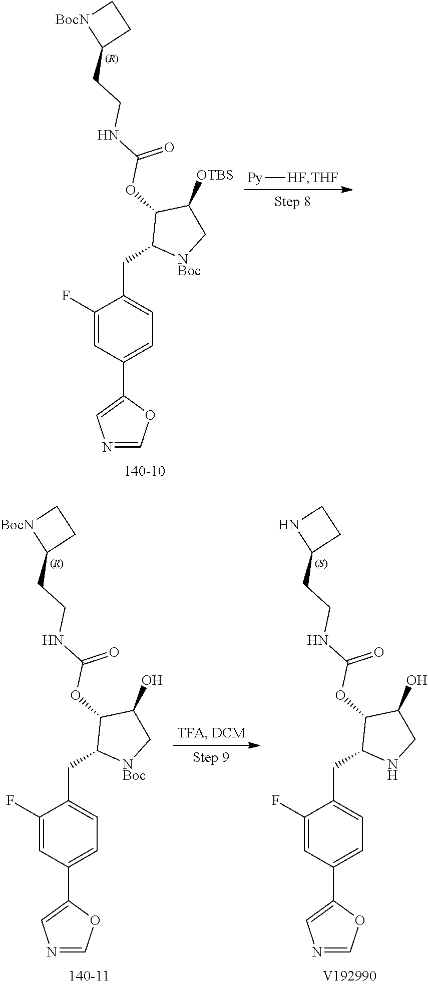

Step 1: To a stirred solution of 3-fluoro-4-iodobenzaldehyde (140-1, 2 g, 8.0 mmol, 1 equiv) in methanol (30 mL) was added TosMIC (2.34 g, 12.0 mmol, 1.5 equiv) and K₂CO₃ (2.21 g, 16.0 mmol, 2 equiv) at r.t. The resulting mixture was stirred at 50° C. for 2 h. The reaction mixture was quenched with water and extracted with EA. The extracts were washed with brine, dried over anhydrous sodium sulfate, filtered and concentrated. The residue was purified by silica gel column chromatography, eluted with PE/EA (2:1) to afford 5-(3-fluoro-4-iodophenyl)-1,3-oxazole (2 g, 86.4% yield) as a yellow solid. MS: m/z: Calc'd for C₉H₅FINO[M+H]⁺290. Found, 290.

Step 2: To a stirred solution of benzyl (2S,3S,4S)-3,4-bis(benzyloxy)-2-(iodomethyl)pyrrolidine-1-carboxylate (140-4, 1000 mg, 1.79 mmol, 1.00 equiv) and Zinc (1172.8 mg, 17.94 mmol, 10 equiv) in DMF (5 mL) was added Iodometric solution (4553.2 mg, 17.94 mmol, 10 equiv) at room temperature under nitrogen atmosphere. The resulting mixture was stirred at room temperature for 1 h under nitrogen atmosphere. The resulting mixture was take the supernatant. To a stirred solution of 5-(3-fluoro-4-iodophenyl)-1,3-oxazole (140-3, 777.8 mg, 2.69 mmol, 1.5 equiv) and Pd₂(dba)₃ (328.5 mg, 0.35 mmol, 0.2 equiv) Q-Phos (255.0 mg, 0.39 mmol, 0.2 equiv) in THF (5 mL) was added supernatant at room temperature under nitrogen atmosphere. The resulting mixture was stirred at room temperature for overnight under nitrogen atmosphere. The resulting mixture was concentrated under reduced pressure. The residue was purified by reversed-phase flash chromatography with the following conditions: column, C18 silica gel; mobile phase, MeCN in Water (0.1% TFA), 0% to 100% gradient in 15 min; detector, UV 254 nm. This resulted in benzyl (2R,3S,4S)-3,4-bis(benzyloxy)-2-{[2-fluoro-4-(1,3-oxazol-5-yl)phenyl]methyl}pyrrolidine-1-carboxylate (400 mg) as a yellow oil. MS: m/z: Calc'd for C₃₆H₃₃FN₂O₅ [M+H]+ 593. found 593.

Step 3: To a stirred solution of benzyl (2R,3S,4S)-3,4-bis(benzyloxy)-2-{[2-fluoro-4-(1,3-oxazol-5-yl)phenyl]methyl}pyrrolidine-1-carboxylate (140-5, 400 mg, 0.67 mmol, 1 equiv) in DCM (10 mL) was added trichloroborane (790.7 mg, 6.75 mmol, 10 equiv) at 0° C. under nitrogen atmosphere. The resulting mixture was stirred at room temperature for overnight. The reaction was quenched with MeOH at 0° C. The resulting mixture was concentrated under vacuum. This resulted in (2R,3S,4S)-2-{[2-fluoro-4-(1,3-oxazol-5-yl)phenyl]methyl}pyrrolidine-3,4-diol (200 mg, 80% purity) as a yellow oil. MS: m/z: Calc'd for C₁₄H1₅FN₂O₃[M+H]+ 279. found 279.

Step 4: To a stirred solution of (2R,3S,4S)-2-{[2-fluoro-4-(1,3-oxazol-5-yl)phenyl]methyl}pyrrolidine-3,4-diol (140-6, 200 mg, 0.71 mmol, 1 equiv) and Et₃N (218.1 mg, 2.15 mmol, 3 equiv) in DCM (5 mL) was added di-tert-butyl dicarbonate (313.7 mg, 1.43 mmol, 2 equiv) at room temperature. The resulting mixture was stirred at room temperature for overnight. The resulting mixture was concentrated under reduced pressure. The residue was purified by reversed-phase flash chromatography with the following conditions: column, C18 silica gel; mobile phase, MeCN in Water (0.1% TFA), 0% to 100% gradient in 10 min; detector, UV 254 nm. This resulted in tert-butyl (2R,3S,4S)-2-{[2-fluoro-4-(1,3-oxazol-5-yl)phenyl]methyl}-3,4-dihydroxypyrrolidine-1-carboxylate (200 mg, 73.5% yield, 90% purity) as a yellow oil. MS: m/z: Calc'd for C₁₉H₂₃FN₂O₅ [M−56]+ 323, found 323.

Step 5: To a stirred solution of tert-butyl (2R,3S,4S)-2-{[2-fluoro-4-(1,3-oxazol-5-yl)phenyl]methyl}-3,4-dihydroxypyrrolidine-1-carboxylate (140-7, 150 mg, 0.39 mmol, 1 equiv) and Imidazole (80.9 mg, 1.18 mmol, 3 equiv) in DCM (10 mL) was added tert-butyl(chloro)dimethylsilane (119.4 mg, 0.79 mmol, 2 equiv) at 0° C. The resulting mixture was stirred at room temperature for overnight. The residue was purified by Prep-TLC (PE/EA 1:1) to afford tert-butyl (2R,3S,4S)-4-[(tert-butyldimethylsilyl)oxy]-2-{[2-fluoro-4-(1,3-oxazol-5-yl)phenyl]methyl}-3-hydroxypyrrolidine-1-carboxylate (80 mg, 40.9% yield, 95% purity) as a white solid. MS: m/z: Calc'd for C₂₅H₃₇FN₂O₅Si [M+H]⁺ 493. found 493.

Step 6: To a stirred solution of tert-butyl (2R,3S,4S)-4-[(tert-butyldimethylsilyl)oxy]-2-{[2-fluoro-4-(1,3-oxazol-5-yl)phenyl]methyl}-3-hydroxypyrrolidine-1-carboxylate (140-8, 80 mg, 0.16 mmol, 1 equiv) and py. (51.3 mg, 0.64 mmol, 4 equiv) in DCM (10 mL) was added 4-nitrophenyl chloroformate (98.1 mg, 0.48 mmol, 3 equiv) at room temperature. The resulting mixture was stirred at room temperature for 2 h. The residue was purified by Prep-TLC (PE/EA 1:1) to afford tert-butyl (2R,3S,4S)-4-[(tert-butyldimethylsilyl)oxy]-2-{[2-fluoro-4-(1,3-oxazol-5-yl)phenyl]methyl}-3-[(4-nitrophenoxycarbonyl)oxy]pyrrolidine-1-carboxylate (90 mg, 84.6% yield, 90% purity) as a white solid. MS: m/z: Calc'd for C₃₂H₄₀FN₃O₉Si [M+H]+ 658. found 658.

Step 7: The compound was prepared in 81.3% yield as an off-white solid according to Substitution reaction for synthesis of Carbamate; General Procedure I using tert-butyl (2R,3S,4S)-4-((tert-butyldimethylsilyl)oxy)-2-(2-fluoro-4-(oxazol-5-yl)benzyl)-3-(((4-nitrophenoxy)carbonyl)oxy)pyrrolidine-1-carboxylate (140-9) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (R)-2-(2-aminoethyl)azetidine-1-carboxylate. MS: m/z: Calc'd for C₃₆H₅₅FN₄O₈Si [M+H]+ 719. found 719.

Step 8: To a stirred solution of tert-butyl (2R,3S,4S)-3-[({2-[(2S)-1-(tert-butoxycarbonyl)azetidin-2-yl]ethyl}carbamoyl)oxy]-4-[(tert-butyldimethylsilyl)oxy]-2-{[2-fluoro-4-(1,3-oxazol-5-yl)phenyl]methyl}pyrrolidine-1-carboxylate (140-10, 40 mg, 0.056 mmol, 1 equiv) in THF (4 mL) was added pyridine hydrofluoride (1 mL) at room temperature. The resulting mixture was stirred at room temperature for overnight. The resulting mixture was filtered, the filter cake was washed with THF (4 mL). The filtrate was concentrated under reduced pressure. This resulted in tert-butyl (2R,3S,4S)-3-[({2-[(2S)-1-(tert-butoxycarbonyl)azetidin-2-yl]ethyl}carbamoyl)oxy]-2-{[2-fluoro-4-(1,3-oxazol-5-yl)phenyl]methyl}-4-hydroxypyrrolidine-1-carboxylate (30 mg, 89.17% yield, 95% purity) as a white solid. MS: m/z: Calc'd for C₃₀H₄₁FN₄O₈ [M−100]+ 505. found 505.

Step 9: The title compound was prepared in 30.1% yield as an off-white solid according to Boc Deprotection; General Procedure II in STEP 9. MS: m/z: Calc'd for C₂₀H₂₅FN₄O₄[M+H]+ 405, found 405. 1H NMR (400 MHz, Methanol-d4) δ 8.82-7.33 (m, 5H), 4.83-4.53 (m, 1H), 4.48-4.25 (m, 1H), 4.15 (s, 2H), 3.90 (s, 2H), 3.15 (s, 6H), 2.59 (s, 1H), 2.40 (s, 1H), 2.05 (s, 2H).

Prep-HPLC-conditions: Column: Welch Ultimate PFP 30*250, 10 μm; Mobile Phase A: Water (0.1% FA), Mobile Phase B: ACN; Flow rate: 60 mL/min mL/min; Gradient: 2% B to 26% B in 10 min; Wave Length: 254 nm/220 nm; RT1(min): 8.5

### Synthetic Procedure of (2R,3S,4S)-4-hydroxy-2-{[4-(1,3-oxazol-5-yl)phenyl]methyl}pyrrolidin-3-yl N-(2-{2-azabicyclo[2.1.1]hexan-1-yl}ethyl)carbamate (V192847)

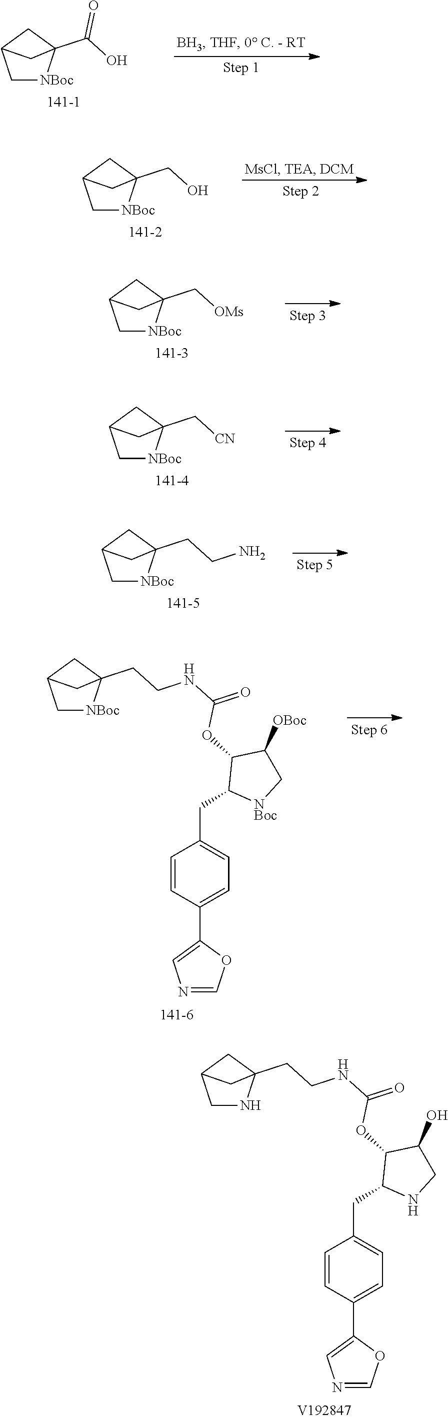

Step 1: To a solution of 2-(tert-butoxycarbonyl)-2-azabicyclo[2.1.1]hexane-1-carboxylic acid (141-1, 500 mg, 2.20 mmol, 1 equiv.) in THF (10 mL) was added borane-tetrahydrofuran complex (1.0M in THF, 4.4 mL, 4.40 mmol, 2 equiv.) at 0° C. The reaction was stirred at room temperature for 3 h. The reaction was quenched by MeOH. Then the reaction mixture was removed under vacuum. The residue was purified by silica gel column chromatography to obtain tert-butyl 1-(hydroxymethyl)-2-azabicyclo[2.1.1]hexane-2-carboxylate (380 mg, 80.9% yield, 100% purity) as a colorless semi-solid. MS: m/z: Calc'd for C₁₁H₁₉NO₃ [M+H−56]⁺ 158. Found, 158.

Step 2: To a solution of tert-butyl 1-(hydroxymethyl)-2-azabicyclo[2.1.1]hexane-2-carboxylate (141-2, 400 mg, 1.87 mmol, 1 equiv.) in DCM (3 mL) was added methanesulfonyl chloride (429.6 mg, 3.75 mmol, 2 equiv.) and Et₃N (569.3 mg, 5.62 mmol, 3 equiv) at 0° C. The reaction was stirred at room temperature for 3 h. The reaction was quenched by water and extracted with DCM. The organic layer combined was washed with brine, then dried over anhydrous sodium sulfate, concentrated. The residue was purified by silica gel column chromatography to obtain tert-butyl 1-[(methanesulfonyloxy)methyl]-2-azabicyclo[2.1.1]hexane-2-carboxylate (350 mg, 64% yield, 92% purity) as a colorless oil. MS: m/z: Calc'd for C₁₂H₂₂NO₅S [M+H]⁺292. Found, 292.

Step 3: To a solution of tert-butyl 1-[(methanesulfonyloxy)methyl]-2-azabicyclo[2.1.1]hexane-2-carboxylate (141-3, 300 mg, 1.03 mmol, 1 equiv.) in N,N-dimethylacetamide (3 mL) were added KCN (201.1 mg, 3.09 mmol, 3 equiv.) and K₂CO₃ (426.9 mg, 3.09 mmol, 3 equiv.). The mixture was stirred at 60° C. overnight. The solid was filtered out and the filtrate was purified by reversed phase column to obtain tert-butyl 1-(cyanomethyl)-2-azabicyclo[2.1.1]hexane-2-carboxylate (55 mg, 24.0% yield, 100% purity) as a colorless oil. MS: m/z: Calc'd for C₁₂H₁₈N₂O₂[M+H]⁺223. Found, 223.

Step 4: To a solution of tert-butyl 1-(cyanomethyl)-2-azabicyclo[2.1.1]hexane-2-carboxylate (141-4, 50 mg, 0.22 mmol, 1 equiv.) in methanol (5 mL) was added Raney-Ni (5 mg, 0.08 mmol, 0.38 equiv.) and NH₃—H₂O (0.2 mL) at N₂ atmosphere. The mixture was stirred at room temperature overnight under H₂ (˜2 bar). The reaction was filtered and washed with MeOH. The filtrate was concentrated under vacuum to afford tert-butyl 1-(2-aminoethyl)-2-azabicyclo[2.1.1]hexane-2-carboxylate (45 mg) as a colorless oil which was not purified further. MS: m/z: Calc'd for C₁₂H₂₂N₂O₂ [M+H−56]⁺171. Found, 171.

### Step 5 and 6

The title compound was prepared in 43.5% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 1-(2-aminoethyl)-2-azabicyclo[2.1.1]hexane-2-carboxylate (141-5) in STEP 5; Boc Deprotection; General Procedure II in STEP 6. MS: m/z: Calc'd for C₂₂H₂₈N₄O₄ [M+H]⁺413. Found, 413. ¹H NMR (400 MHz, Methanol-d₄) δ 8.28 (s, 1H), 7.79-7.73 (m, 2H), 7.55 (s, 1H), 7.45 (d, J=8.1 Hz, 2H), 5.01 (dd, J=3.8, 1.4 Hz, 1H), 4.40 (d, J=3.9 Hz, 1H), 4.31-4.20 (m, 1H), 3.60 (dd, J=12.7, 4.3 Hz, 1H), 3.39 (s, 2H), 3.37-3.34 (m, 2H), 3.29-3.18 (m, 2H), 3.11 (dd, J=14.1, 8.5 Hz, 1H), 2.92 (t, J=3.3 Hz, 1H), 2.21-2.11 (m, 4H), 1.63-1.51 (m, 2H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1(min): 8.5.

### Synthetic Procedure of (2R,3S,4S)-4-hydroxy-2-{[4-(1,3-oxazol-5-yl)phenyl]methyl}pyrrolidin-3-yl N-(2-{2-azabicyclo[2.1.1]hexan-4-yl}ethyl)carbamate (V192849)

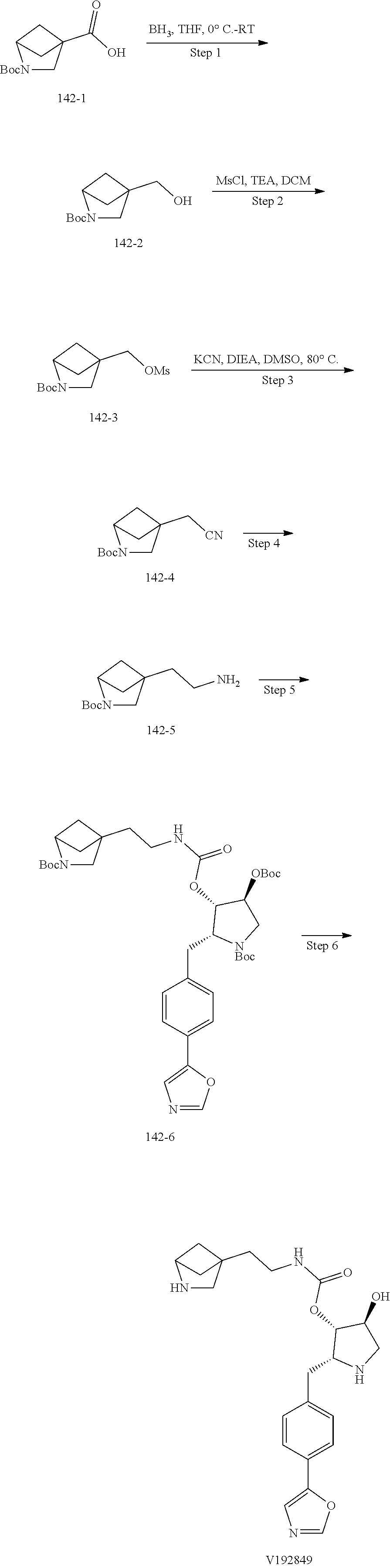

Step 1: To a solution of 2-(tert-butoxycarbonyl)-2-azabicyclo[2.1.1]hexane-4-carboxylic acid (142-1, 500 mg, 2.20 mmol, 1 equiv.) in THF (10 mL) was added borane-tetrahydrofuran complex (1.0M in THF, 4.4 mL, 2.00 equiv.) at 0° C. The reaction was stirred at room temperature overnight. The mixture was quenched by MeOH and the solvent was removed under vacuum. The residue was purified by silica gel column chromatographic to obtain tert-butyl 4-(hydroxymethyl)-2-azabicyclo[2.1.1]hexane-2-carboxylate (350 mg, 74.5% yield, 90% purity) as a colorless oil. MS: m/z: Calc'd for C₁₁H₁₉NO₃ [M+H−56]⁺158. Found, 158.

Step 2: To a solution of tert-butyl 4-(hydroxymethyl)-2-azabicyclo[2.1.1]hexane-2-carboxylate (142-2, 360 mg, 1.68 mmol, 1 equiv.) in DCM (5 mL) was added methanesulfonyl chloride (387 mg, 3.37 mmol, 2 equiv.) and Et₃N (512.4 mg, 5.06 mmol, 3 equiv.) at 0° C. The reaction was stirred at room temperature overnight. The mixture was diluted with water and extracted with DCM, washed with brine. The organic layer was dried over sodium sulfate, filtered and concentrated. The residue was purified by silica gel column chromatographic to obtain tert-butyl 4-[(methanesulfonyloxy)methyl]-2-azabicyclo[2.1.1]hexane-2-carboxylate (253 mg, 51% yield, 90% purity) as a colorless semi-solid. MS: m/z: Calc'd for C₁₂H₂₂NO₅S [M+H−56]⁺236. Found, 236.

Step 3: To a solution of tert-butyl 4-[(methanesulfonyloxy)methyl]-2-azabicyclo[2.1.1]hexane-2-carboxylate (142-3, 253 mg, 0.86 mmol, 1 equiv.) in DMSO (3 mL) was added KCN (170 mg, 2.60 mmol, 3 equiv.) and DIEA (337 mg, 2.60 mmol, 3 equiv). The reaction was stirred at 80° C. overnight.

The mixture was diluted with water and extracted with DCM, washed with brine. The organic layer was dried over sodium sulfate, filtered and concentrated. The residue was purified by silica gel column chromatographic to obtain tert-butyl 4-(cyanomethyl)-2-azabicyclo[2.1.1]hexane-2-carboxylate (160 mg, 82.8% yield) as an off-white semi-solid. MS: m/z: Calc'd for C₁₂H₁₈N₂O₂ [M+H−56]⁺167. Found, 167.

Step 4: To a solution of tert-butyl 4-(cyanomethyl)-2-azabicyclo[2.1.1]hexane-2-carboxylate (142-4, 150 mg, 0.67 mmol, 1 equiv) in methanol (5 mL) was added Raney-Ni (19.8 mg, 0.33 mmol, 0.5 equiv) and NH₃. H₂O (23.6 mg, 0.67 mmol, 1 equiv). The reaction was stirred at room temperature overnight. The mixture was filtered and the filtrate cake was washed with MeOH. The filtrate was concentrated under vacuum to afford tert-butyl 4-(2-aminoethyl)-2-azabicyclo[2.1.1]hexane-2-carboxylate (150 mg) which was used directly in the next step without further purification. MS: m/z: Calc'd for C₁₂H₂₂N₂O₂ [M+H]⁺227. Found, 227.

### Step 5 and 6

The title compound was prepared in 24.6% overall yield as a light-yellow solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl 4-(2-aminoethyl)-2-azabicyclo[2.1.1]hexane-2-carboxylate (142-5) in STEP 5; Boc Deprotection; General Procedure II in STEP 6. MS: m/z: Calc'd for C₂₂H₂₈N₄O₄ [M+H]⁺413. Found, 413. ¹H NMR (400 MHz, Methanol-d₄) δ 8.27 (s, 1H), 7.79-7.72 (m, 2H), 7.54 (s, 1H), 7.48-7.41 (m, 2H), 5.00 (d, J=3.5 Hz, 1H), 4.42-4.37 (m, 1H), 4.30-4.22 (m, 1H), 4.18 (t, J=1.7 Hz, 1H), 3.68-3.55 (m, 1H), 3.33-3.17 (m, 6H), 3.10 (dd, J=14.2, 8.5 Hz, 1H), 2.05 (d, J=6.0 Hz, 2H), 1.99 (t, J=7.3 Hz, 2H), 1.65-1.53 (m, 2H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 20% B in 10 min; Wave Length: 254 nm/220 nm; RT1 (min): 8.5.

### Synthetic Procedure of (2R,3S,4S)-4-hydroxy-2-{[4-(1,3-oxazol-5-yl)phenyl]methyl}pyrrolidin-3-yl N-{2-[(2S,5S)-5-methylpyrrolidin-2-yl]ethyl}carbamate (V192850)

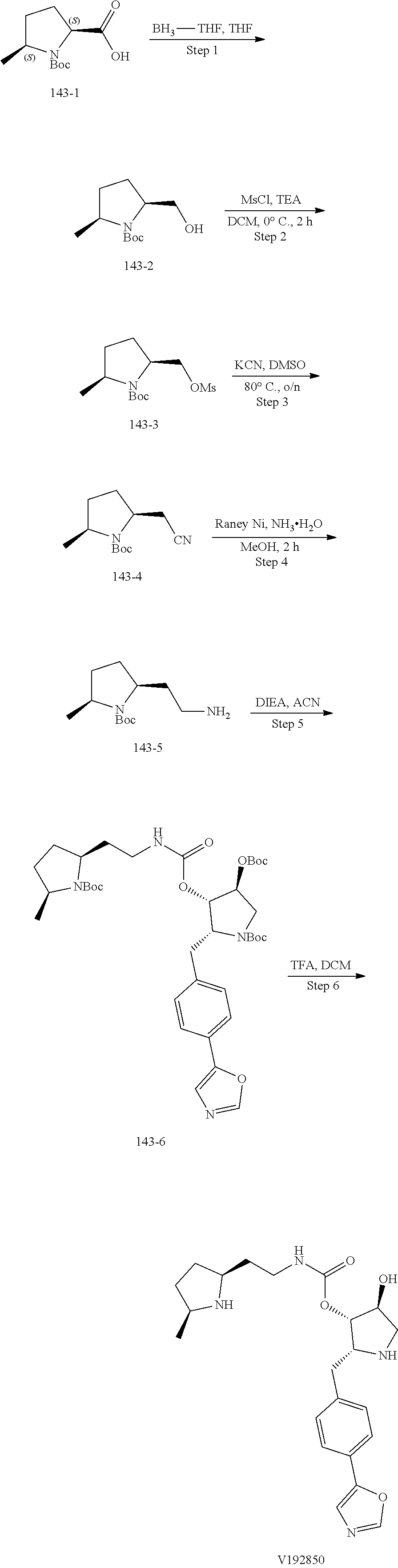

Step 1: To a stirred solution of (2S,5S)-1-(tert-butoxycarbonyl)-5-methylpyrrolidine-2-carboxylic acid (143-1, 1 g, 4.36 mmol, 1 equiv) in THF (8 mL) were added borane-tetrahydrofuran complex (1.0 M in THF, 8 mL, 8 mmol, 1.84 equiv.) at 0° C. The above mixture was stirred at room temperature overnight. The resulting mixture was concentrated under reduced pressure. The residue was purified by silica gel column chromatography, eluted with PE/EA (3:1) to afford tert-butyl (2S,5S)-2-(hydroxymethyl)-5-methylpyrrolidine-1-carboxylate (800 mg, 85.2% yield) as a light yellow oil. MS: m/z: Calc'd for C₁₁H₂₁NO₃ [M+H−56]⁺160. Found, 160.

Step 2: To a stirred solution of tert-butyl (2S,5S)-2-(hydroxymethyl)-5-methylpyrrolidine-1-carboxylate (143-2, 600 mg, 2.78 mmol, 1 equiv) and methanesulfonyl chloride (638 mg, 5.57 mmol, 2 equiv) in DCM (8 mL) was added Et₃N (846 mg, 8.36 mmol, 3 equiv) at 0° C. The resulting mixture was stirred at room temperature for 2 h. The resulting mixture was concentrated under reduced pressure. The residue was purified by silica gel column chromatography, eluted with PE/EA (3:1) to afford tert-butyl (2S,5S)-2-[(methanesulfonyloxy)methyl]-5-methylpyrrolidine-1-carboxylate (800 mg, 97.8% yield, 95% purity) as a light yellow oil. MS: m/z: Calc'd for C₁₂H₂₃NO₅S [M+H−56]⁺238. Found, 238.

Step 3: To a stirred solution of tert-butyl (2S,5S)-2-[(methanesulfonyloxy)methyl]-5-methylpyrrolidine-1-carboxylate (143-3, 790 mg, 2.69 mmol, 1 equiv) and KCN (351 mg, 5.38 mmol, 2 equiv) in DMSO (8 mL) at room temperature. The above mixture was stirred at 80° C. overnight. The resulting mixture was diluted with EtOAc, washed with brine, dried over anhydrous Na₂SO₄. After filtration, the filtrate was concentrated under reduced pressure. The residue was purified by silica gel column chromatography, eluted with PE/EA (6:1) to afford tert-butyl (2S,5S)-2-(cyanomethyl)-5-methylpyrrolidine-1-carboxylate (370 mg, 61% yield, 95% purity) as a light yellow oil. MS: m/z: Calc'd for C₁₂H₂₀N₂O₂ [M+H−56]⁺ 169. Found, 169.

Step 4: To a stirred solution of tert-butyl (2S,5S)-2-(cyanomethyl)-5-methylpyrrolidine-1-carboxylate (143-4, 365 mg, 1.62 mmol, 1 equiv) and Raney nickel (286.5 mg, 4.88 mmol, 3 equiv) in MeOH (6 mL) were added ammonium hydroxide (9% in water) (85.5 mg, 2.44 mmol, 1.5 equiv) at room temperature. The resulting mixture was stirred at room temperature for 2 h. The precipitated solids were collected by filtration and washed with MeOH (6 mL). The filtrate was concentrated under reduced pressure to afford tert-butyl (2S,5S)-2-(2-aminoethyl)-5-methylpyrrolidine-1-carboxylate (250 mg) as a light yellow oil. MS: m/z: Calc'd for C₁₂H₂₄N₂O₂ [M+H]⁺229. Found, 229.

### Step 5 and 6

The title compound was prepared in 23.1% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-oxazol-5-yl)phenyl]methyl}pyrrolidine-1-carboxylate (Int-10) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), bis(tert-butyl (2S,5S)-2-(2-aminoethyl)-5-methylpyrrolidine-1-carboxylate) (143-5) in STEP 5; Boc Deprotection; General Procedure II in STEP 6. MS: m/z: Calc'd for C₂₂H₃₀N₄O₄ [M+H]⁺415. found 415. ¹H NMR (400 MHz, Methanol-d₄) δ δ 8.28 (s, 1H), 7.80-7.73 (m, 2H), 7.54 (s, 1H), 7.45 (d, J=8.2 Hz, 2H), 5.05-4.99 (m, 1H), 4.41 (d, J=4.0 Hz, 1H), 4.33-4.24 (m, 1H), 3.71-3.64 (m, 1H), 3.61-3.51 (m, 2H), 3.32-3.18 (m, 4H), 3.10 (dd, J=14.2, 8.6 Hz, 1H), 2.29 (dd, J=16.1, 8.2 Hz, 2H), 2.09-1.83 (m, 2H), 1.77 (dd, J=22.9, 9.3 Hz, 2H), 1.43 (d, J=6.6 Hz, 3H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min mL/min; Gradient: 2% B to 23% B in 10 min; Wave Length: 254 nm/220 nm; RT1(min): 9.5.

### Synthetic Procedure of (2R,3S,4S)-2-{[2-fluoro-4-(1,3-oxazol-5-yl)phenyl]methyl}-4-hydroxypyrrolidin-3-ylN-[(3-fluorophenyl)methyl]carbamate (V192861)

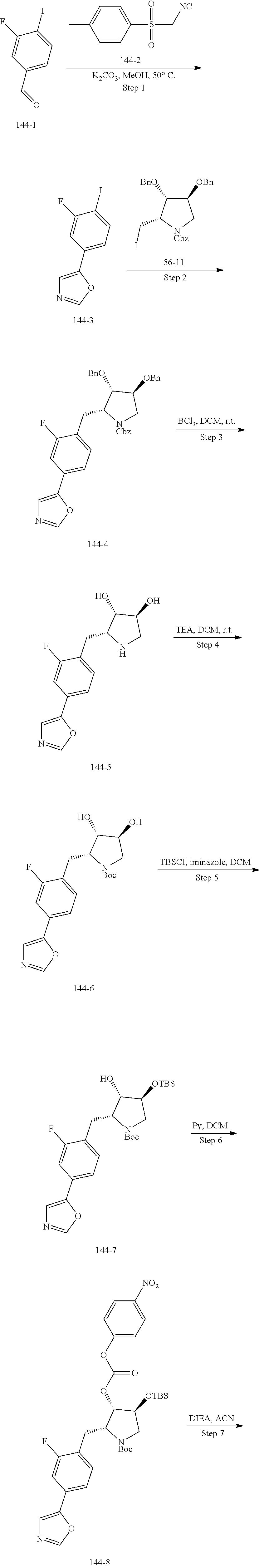

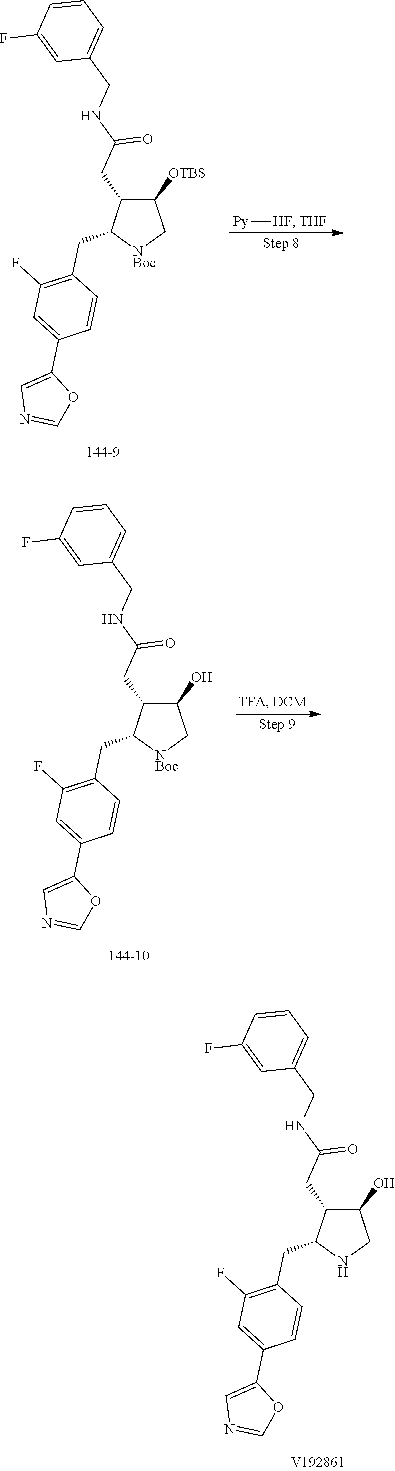

Step 1: A mixture of 3-fluoro-4-iodobenzaldehyde (144-1, 2 g, 8.0 mmol, 1 equiv.), TosMIC (144-2, 2.34 g, 12.0 mmol, 1.5 equiv.) and K₂CO₃ (2.21 g, 16.00 mmol, 2 equiv.) in methanol (30 mL) was stirred at 50° C. for 2 h under nitrogen atmosphere. The resulting mixture was concentrated under vacuum. The residue was diluted with water and extracted with CHCl₃. The combined organic layers were dried over anhydrous Na₂SO₄. After filtration, the filtrate was concentrated under reduced pressure. The residue was purified by silica gel column chromatography, eluted with PE/EA (2:1) to afford 5-(3-fluoro-4-iodophenyl)-1,3-oxazole (2 g, 86.4% yield, 99.9% purity) as a yellow solid.

Step 2: To a stirred mixture of benzyl (2S,3S,4S)-3,4-bis(benzyloxy)-2-(iodomethyl)pyrrolidine-1-carboxylate (56-11, 1000 mg, 1.79 mmol, 1.00 equiv.) and zinc (1172.8 mg, 17.94 mmol, 10 equiv.) in DMF (5 mL) was added iodometric solution (4553.2 mg, 17.94 mmol, 10 equiv.) at room temperature under nitrogen atmosphere. The above mixture was stirred at room temperature for 1 h under nitrogen atmosphere. The resulting mixture was take the supernatant. To a stirred mixture of 5-(3-fluoro-4-iodophenyl)-1,3-oxazole (144-3, 777.8 mg, 2.69 mmol, 1.5 equiv.) and Pd₂(dba)₃ (328.5 mg, 0.35 mmol, 0.2 equiv.) and Q-Phos (255.0 mg, 0.39 mmol, 0.2 equiv.) in THF (5 mL) was added supernatant at room temperature under nitrogen atmosphere. The mixture was stirred at room temperature overnight under nitrogen atmosphere. The resulting mixture was concentrated under reduced pressure. The residue was purified by reversed-phase flash chromatography to afford benzyl (2R,3S,4S)-3,4-bis(benzyloxy)-2-{[2-fluoro-4-(1,3-oxazol-5-yl)phenyl]methyl}pyrrolidine-1-carboxylate (400 mg) as a yellow oil. MS: m/z: Calc'd for C₃₆H₃₃FN₂O₅ [M+H]⁺593. found 593.

Step 3: To a stirred mixture of benzyl (2R,3S,4S)-3,4-bis(benzyloxy)-2-{[2-fluoro-4-(1,3-oxazol-5-yl)phenyl]methyl}pyrrolidine-1-carboxylate (144-4, 400 mg, 0.67 mmol, 1 equiv.) in DCM (10 mL) was added trichloroborane (790.7 mg, 6.75 mmol, 10 equiv.) at 0° C. under nitrogen atmosphere. The reaction mixture was stirred at room temperature overnight under nitrogen atmosphere. The reaction was quenched with MeOH at 0° C. The resulting mixture was concentrated under vacuum to afford (2R,3S,4S)-2-{[2-fluoro-4-(1,3-oxazol-5-yl)phenyl]methyl}pyrrolidine-3,4-diol (200 mg, 80% purity) as a yellow oil. MS: m/z: Calc'd for C₁₄H1₅FN₂O₃[M+H]⁺279. found 279.

Step 4: To a stirred mixture of (2R,3S,4S)-2-{[2-fluoro-4-(1,3-oxazol-5-yl)phenyl]methyl}pyrrolidine-3,4-diol (144-5, 200 mg, 0.71 mmol, 1 equiv.) and Et₃N (218 mg, 2.15 mmol, 3 equiv.) in DCM (5 mL) was added di-tert-butyl dicarbonate (314 mg, 1.43 mmol, 2 equiv.) at room temperature. The above mixture was stirred at room temperature overnight. The resulting mixture was concentrated under reduced pressure. The residue was purified by reversed-phase flash chromatography to afford tert-butyl (2R,3S,4S)-2-{[2-fluoro-4-(1,3-oxazol-5-yl)phenyl]methyl}-3,4-dihydroxypyrrolidine-1-carboxylate (200 mg, 73.5% yield, 90% purity) as a yellow oil.

Step 5: To a stirred mixture of tert-butyl (2R,3S,4S)-2-{[2-fluoro-4-(1,3-oxazol-5-yl)phenyl]methyl}-3,4-dihydroxypyrrolidine-1-carboxylate (144-6, 150 mg, 0.39 mmol, 1 equiv.) and imidazole (80.9 mg, 1.18 mmol, 3 equiv.) in DCM (10 mL) was added tert-butyl(chloro)dimethylsilane (119.4 mg, 0.79 mmol, 2 equiv.) at 0° C. The resulting mixture was stirred at room temperature overnight. The residue was purified by Prep-TLC (PE/EA, 1:1) to afford tert-butyl (2R,3S,4S)-4-[(tert-butyldimethylsilyl)oxy]-2-{[2-fluoro-4-(1,3-oxazol-5-yl)phenyl]methyl}-3-hydroxypyrrolidine-1-carboxylate (80 mg, 40.9% yield, 95% purity) as a white solid. MS: m/z: Calc'd for C₂₅H₃₇FN₂O₅Si [M+H]⁺493. found 493.

Step 6: To a stirred mixture of tert-butyl (2R,3S,4S)-4-[(tert-butyldimethylsilyl)oxy]-2-{[2-fluoro-4-(1,3-oxazol-5-yl)phenyl]methyl}-3-hydroxypyrrolidine-1-carboxylate (144-7, 80 mg, 0.16 mmol, 1 equiv.) and pyridine (51.3 mg, 0.64 mmol, 4 equiv.) in DCM (10 mL) was added 4-nitrophenyl chloroformate (98.1 mg, 0.48 mmol, 3 equiv.) at room temperature. The above mixture was stirred at room temperature for 2 h.

The resulting mixture was concentrated. The residue was purified by Prep-TLC (PE/EA, 1:1) to afford tert-butyl (2R,3S,4S)-4-[(tert-butyldimethylsilyl)oxy]-2-{[2-fluoro-4-(1,3-oxazol-5-yl)phenyl]methyl}-3-[(4-nitrophenoxycarbonyl)oxy]pyrrolidine-1-carboxylate (90 mg, 84.6% yield, 90% purity) as a white solid. MS: m/z: Calc'd for C₃₂H₄₀FN₃O₉Si [M+H]⁺ 658. found 658.

### Step 7, 8, and 9

The title compound was prepared in 38.7% overall yield as a white solid according to Substitution reaction for synthesis of Carbamate; General Procedure I using tert-butyl (2R,3S,4S)-4-[(tert-butyldimethylsilyl)oxy]-2-{[2-fluoro-4-(1,3-oxazol-5-yl)phenyl]methyl}-3-[(4-nitrophenoxycarbonyl)oxy]pyrrolidine-1-carboxylate (144-8) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 7; TBS Deprotection; General Procedure VII in STEP 8; Boc Deprotection; General Procedure II in STEP 9. MS: m/z: Calc'd for C₂₂H₂₁F₂N₃O₄[M+H]⁺430. found 430. ¹H NMR (400 MHz, Methanol-d₄) δ 8.31 (s, 1H), 7.60 (s, 1H), 7.58-7.48 (m, 2H), 7.45-7.33 (m, 2H), 7.16 (d, J=7.7 Hz, 1H), 7.13-7.00 (m, 2H), 5.00-4.94 (m, 1H), 4.45-4.36 (m, 2H), 4.35-4.24 (m, 2H), 3.62 (dd, J=12.6, 4.3 Hz, 1H), 3.28-2.97 (m, 3H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 12% B to 42% B in 10 min; Wave Length: 254 nm/220 nm; RT1(min): 8.5.

### Synthetic Procedure of (2R,3S,4S)-4-hydroxy-2-{[6-(1,3-oxazol-5-yl)pyridin-3-yl]methyl}pyrrolidin-3-yl N-[(3-fluorophenyl)methyl]carbamate (V192862)

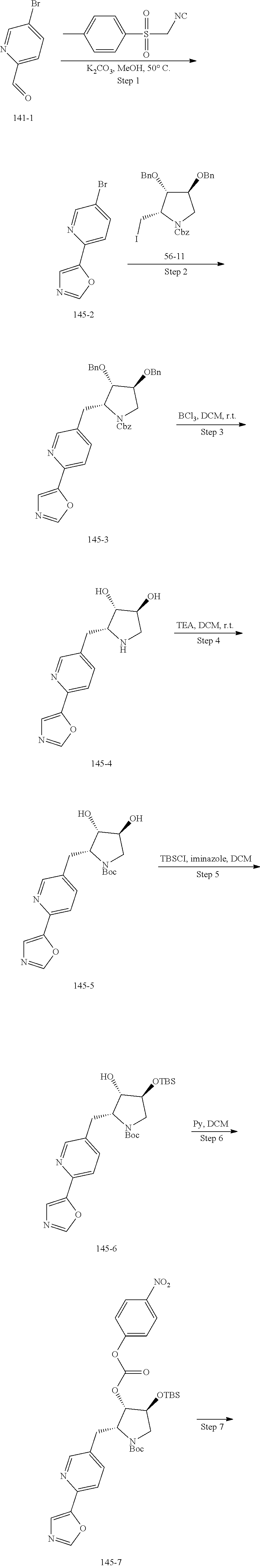

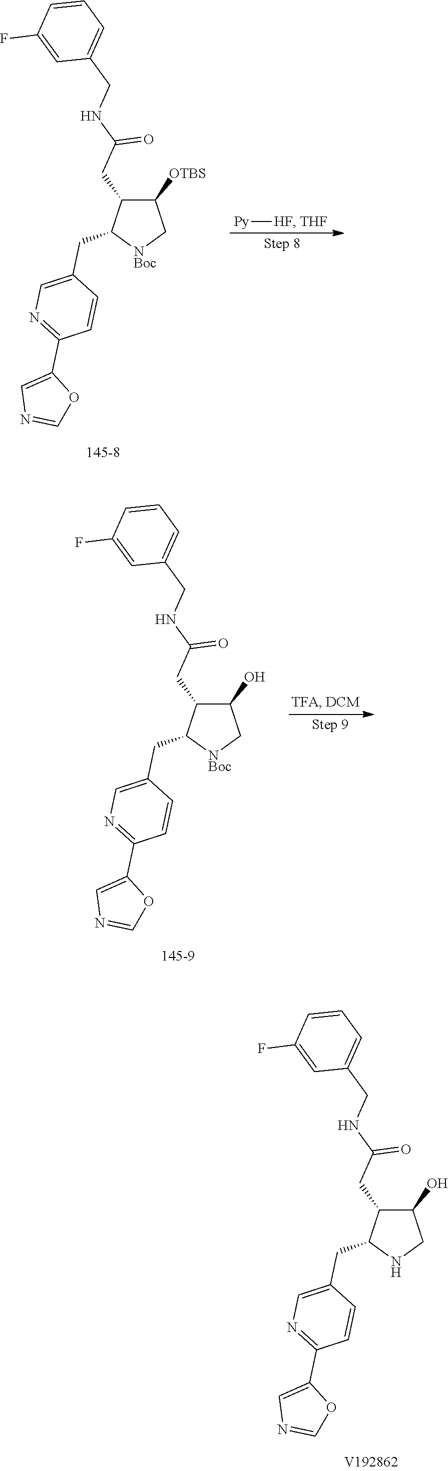

Step 1: A mixture of 5-bromopyridine-2-carbaldehyde (145-1, 2 g, 10.75 mmol, 1 equiv.), TosMIC (3.15 g, 16.12 mmol, 1.5 equiv.) and K₂CO₃ (2.97 g, 21.50 mmol, 2 equiv.) in methanol (30 mL) was stirred at 50° C. for 2 h under nitrogen atmosphere. The resulting mixture was concentrated under vacuum. The residue was diluted with water and extracted with CH₂Cl₂. The combined organic layers were dried over anhydrous Na₂SO₄. After filtration, the filtrate was concentrated under reduced pressure. The residue was purified by silica gel column chromatography, eluted with PE/EA (1:1) to afford 5-bromo-2-(1,3-oxazol-5-yl)pyridine (2.2 g, 90.9% yield, 100% purity) as a yellow solid.

Step 2: To a stirred mixture of benzyl (2S,3S,4S)-3,4-bis(benzyloxy)-2-(iodomethyl)pyrrolidine-1-carboxylate (56-11, 1000 mg, 1.79 mmol, 1.00 equiv.) and zinc (1172.8 mg, 17.94 mmol, 10 equiv.) in DMF (5 mL) was added iodometric solution (4553.2 mg, 17.94 mmol, 10 equiv.) at room temperature under nitrogen atmosphere. The above mixture was stirred at room temperature for 1 h under nitrogen atmosphere. The resulting mixture was take the supernatant. To a stirred mixture of 5-bromo-2-(1,3-oxazol-5-yl)pyridine (145-2, 605.58 mg, 2.691 mmol, 1.5 equiv.), Pd₂(dba)₃ (328.5 mg, 0.35 mmol, 0.2 equiv.) and Q-Phos (255.0 mg, 0.36 mmol, 0.2 equiv.) in THF (5 mL) was added supernatant at room temperature under nitrogen atmosphere. The mixture was stirred at room temperature overnight under nitrogen atmosphere. The resulting mixture was concentrated under reduced pressure. The residue was purified by reversed-phase flash chromatography with the following conditions: column, C18 silica gel; mobile phase, MeCN in Water (0.1% TFA), 0% to 100% gradient in 15 min; detector, UV 254 nm. Pure fractions were evaporated to afford benzyl (2R,3S,4S)-3,4-bis(benzyloxy)-2-{[6-(1,3-oxazol-5-yl)pyridin-3-yl]methyl}pyrrolidine-1-carbo-xylate (400 mg, 38.7% yield, 50% purity) as a yellow oil.

Step 3: To a stirred mixture of benzyl (2R,3S,4S)-3,4-bis(benzyloxy)-2-{[6-(1,3-oxazol-5-yl)pyridin-3-yl]methyl}pyrrolidine-1-carboxylate (145-3, 400 mg, 0.69 mmol, 1 equiv.) in DCM (10 mL) was added trichloroborane (814.0 mg, 6.95 mmol, 10 equiv.) at 0° C. under nitrogen atmosphere. The mixture was stirred at room temperature overnight under nitrogen atmosphere. The reaction was quenched with MeOH at 0° C. The resulting mixture was concentrated under vacuum to afford (2R,3S,4S)-2-{[6-(1,3-oxazol-5-yl)pyridin-3-yl]methyl}pyrrolidine-3,4-diol (200 mg, 80% purity) as a yellow oil. MS: m/z: Calc'd for C₁₃H1₅N₃O₃ [M+H]⁺262. found 262.

Step 4: To a stirred mixture of (2R,3S,4S)-2-{[6-(1,3-oxazol-5-yl)pyridin-3-yl]methyl}pyrrolidine-3,4-diol (145-4, 200 mg, 0.76 mmol, 1 equiv.) and Et₃N (232.3 mg, 2.29 mmol, 3 equiv.) in DCM (5 mL) was added di-tert-butyl dicarbonate (334.1 mg, 1.53 mmol, 2 equiv.) at room temperature. The above mixture was stirred at room temperature overnight. The resulting mixture was concentrated under reduced pressure. The residue was purified by reversed-phase flash chromatography with the following conditions: column, C18 silica gel; mobile phase, MeCN in Water (0.1% TFA), 0% to 100% gradient in 10 min; detector, UV 254 nm. Pure fractions were evaporated to afford tert-butyl (2R,3S,4S)-3,4-dihydroxy-2-{[6-(1,3-oxazol-5-yl)pyridin-3-yl]methyl}pyrrolidine-1-carboxylate (200 mg, 72% yield, 90% purity) as a yellow oil. MS: m/z: Calc'd for C₁₈H₂₃N₃O₅ [M+H]⁺ 362. found 362.

Step 5: To a stirred mixture of tert-butyl (2R,3S,4S)-3,4-dihydroxy-2-{[6-(1,3-oxazol-5-yl)pyridin-3-yl]methyl}pyrrolidine-1-carboxylate (145-5, 150 mg, 0.415 mmol, 1 equiv.) and imidazole (84.7 mg, 1.24 mmol, 3 equiv.) in DCM (10 mL) was added tert-butyl(chloro)dimethylsilane (125.1 mg, 0.83 mmol, 2 equiv.) at 0° C. The resulting mixture was stirred at room temperature overnight and concentrated. The residue was purified by Prep-TLC (PE/EA, 1:1) to afford tert-butyl (2R,3S,4S)-4-[(tert-butyldimethylsilyl)oxy]-3-hydroxy-2-{[6-(1,3-oxazol-5-yl)pyridin-3-yl]methyl}pyrrolidine-1-carboxylate (50 mg, 25.3% yield, 95% purity) as a white solid. MS: m/z: Calc'd for C₂₄H₃₇N₃O₅Si [M+H]⁺ 76. found 476.

Step 6: To a stirred mixture of tert-butyl (2R,3S,4S)-4-[(tert-butyldimethylsilyl)oxy]-3-hydroxy-2-{[6-(1,3-oxazol-5-yl)pyridin-3-yl]methyl}pyrrolidine-1-carboxylate (145-6, 30 mg, 0.06 mmol, 1 equiv.) and pyridine (19.9 mg, 0.25 mmol, 4 equiv.) in DCM (10 mL) was added 4-nitrophenyl chloroformate (38.14 mg, 0.189 mmol, 3 equiv.) at 0° C. The above mixture was stirred at room temperature overnight. The resulting mixture was concentrated under reduced pressure. The residue was purified by Prep-TLC (PE/EA, 1:1) to afford tert-butyl (2R,3S,4S)-4-[(tert-butyldimethylsilyl)oxy]-3-[(4-nitrophenoxycarbonyl)oxy]-2-{[6-(1,3-oxazol-5-yl)pyridin-3-yl]methyl}pyrrolidine-1-carboxylate (30 mg, 74% yield, 90% purity) as a white solid. MS: m/z: Calc'd for C₃₁H₄₀N₄O₉Si [M+H]⁺641. found 641.

### Step 7, 8, and 9

The title compound was prepared in 11.5% overall yield as a white solid according to Substitution reaction for synthesis of Carbamate; General Procedure I using tert-butyl (2R,3S,4S)-4-[(tert-butyldimethylsilyl)oxy]-3-[(4-nitrophenoxycarbonyl)oxy]-2-{[6-(1,3-oxazol-5-yl)pyridin-3-yl]methyl}pyrrolidine-1-carboxylate (145-7) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 7; TBS Deprotection; General Procedure VII in STEP 8; Boc Deprotection; General Procedure II in STEP 9. MS: m/z: Calc'd for C₂₁H₂₁FN₄O₄[M+H]⁺413. found 413. ¹H NMR (400 MHz, Methanol-d₄) δ 8.62-8.57 (m, 1H), 8.35 (s, 1H), 7.95-7.88 (m, 1H), 7.75 (d, J=7.5 Hz, 2H), 7.42-7.32 (m, 1H), 7.17 (d, J=7.7 Hz, 1H), 7.10 (d, J=9.7 Hz, 1H), 7.06-6.97 (m, 1H), 5.03-4.98 (m, 1H), 4.44-4.38 (m, 1H), 4.38-4.24 (m, 3H), 3.77-3.69 (m, 1H), 3.31-3.21 (m, 3H).

Prep-HPLC-conditions: Column: XBridge Prep OBD C18 Column, 30*150 mm, 5 μm; Mobile Phase A: Water (0.1% FA), Mobile Phase B: ACN; Flow rate: 60 mL/min; Gradient: 6% B to 36% B in 10 min; Wave Length: 254 nm/220 nm; RT1(min): 8.5.

### Synthetic Procedure of (2R,3S,4S)-4-hydroxy-2-({4-[6-(trifluoromethyl)imidazo[2,1-b][1,3]thiazol-2-yl]phenyl}methyl)pyrrolidin-3-yl N-[(3-fluorophenyl)methyl]carbamate (V192874)

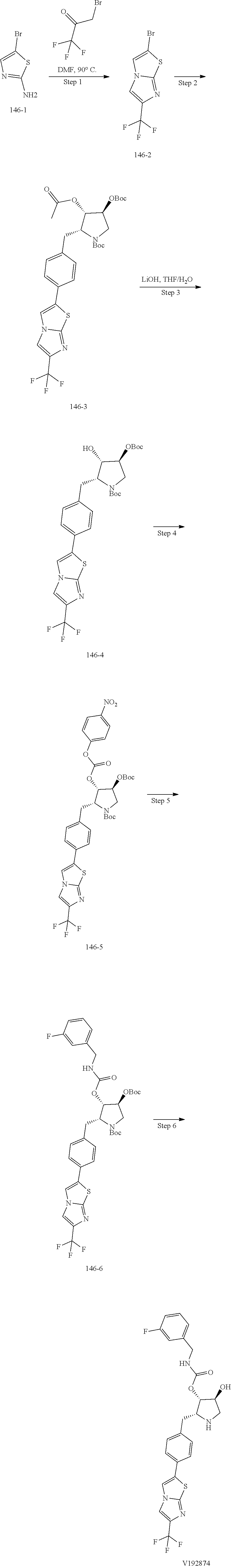

Step 1: To a stirred mixture of 3-bromo-1,1,1-trifluoropropan-2-one (146-1, 6.2 g, 32.47 mmol, 1 equiv.) in DMF (100 mL) was added 5-bromo-1,3-thiazol-2-amine (5.81 g, 32.47 mmol, 1 equiv.) at room temperature under nitrogen atmosphere. The resulting mixture was stirred at 90° C. overnight under nitrogen atmosphere. The reaction mixture was quenched with water and extracted with EA. The extracts were washed with brine, dried over anhydrous sodium sulfate, filtered and concentrated. The residue was purified by reversed-phase flash chromatography with the following conditions: column, C18 silica gel; mobile phase, MeCN in Water (0.1% TFA), 10% to 50% gradient in 10 min; detector, UV 254 nm. Pure fractions were evaporated to afford 2-bromo-6-(trifluoromethyl)imidazo[2,1-b][1,3]thiazole (6 g, 68% yield, 95% purity) as a yellow solid.

Step 2: To a stirred mixture of 2-bromo-6-(trifluoromethyl)imidazo[2,1-b][1,3]thiazole (146-2, 339.3 mg, 1.25 mmol, 2 equiv), 4-{[(2R,3S,4S)-3-(acetyloxy)-1-(tert-butoxycarbonyl)-4-[(tert-butoxycarbonyl)oxy]pyrrolidin-2-yl]methyl}phenylboronic acid (111-1, 300 mg, 0.62 mmol, 1.00 equiv.) and Na₂CO₃ (132.6 mg, 1.25 mmol, 2 equiv.) in 1,4-dioxane (10 mL) and water (1 mL) was added Pd(dtbpf)Cl₂ (40.79 mg, 0.06 mmol, 0.1 equiv.) at room temperature under nitrogen atmosphere. The above mixture was stirred at 60° C. overnight under nitrogen atmosphere. The resulting mixture was concentrated under reduced pressure. The residue was purified by reversed-phase flash chromatography with the following conditions: column, C18 silica gel; mobile phase, MeCN in Water (0.1% TFA), 0% to 100% gradient in 10 min; detector, UV 254 nm. Pure fractions were evaporated to afford tert-butyl (2R,3S,4S)-3-(acetyloxy)-4-[(tert-butoxycarbonyl)oxy]-2-({4-[6-(trifluoromethyl)imidazo[2,1-b][1,3]thiazol-2-yl]phenyl}methyl)pyrrolidine-1-carboxylate (350 mg, 89% yield, 90% purity) as a white solid. MS: m/z: Calc'd for C₂₉H₃₄F₃N₃O₇S [M+H]⁺626. found 626.

Step 3: The compound was prepared in 89% yield as a white solid according to Hydrolysis reaction with LiOH; General Procedure V. MS: m/z: Calc'd for C₂₇H₃₂F₃N₃O₆S [M+H]⁺584. found 584.

Step 4: To a stirred mixture of tert-butyl (2R,3S,4S)-4-[(tert-butoxycarbonyl)oxy]-3-hydroxy-2-({4-[6-(trifluoromethyl)imidazo[2,1-b][1,3]thiazol-2-yl]phenyl}methyl)pyrrolidine-1-carboxylate (146-4, 400 mg, 0.68 mmol, 1 equiv.) and pyridine (162.6 mg, 2.05 mmol, 3 equiv.) in DCM (10 mL) was added 4-nitrophenyl chloroformate (207.2 mg, 1.02 mmol, 1.5 equiv.) at 0° C. The above mixture was stirred at room temperature for 1 h. The resulting mixture was concentrated under reduced pressure. The residue was purified by Prep-TLC (PE/EA, 4:1) to afford tert-butyl (2R,3S,4S)-4-[(tert-butoxycarbonyl)oxy]-3-[(4-nitrophenoxycarbonyl)oxy]-2-({4-[6-(trifluoromethyl)imidazo[2,1-b][1,3]thiazol-2-yl]phenyl}methyl)pyrrolidine-1-carboxylate (400 mg, 77.9% yield, 90% purity) as a yellow solid. MS: m/z: Calc'd for C₃₄H₃₅F₃N₄O₁₀S [M+H]⁺ 749. found 749.

### Step 5 and 6

The title compound was prepared in 51.7% overall yield as a white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-({4-[6-(trifluoromethyl)imidazo[2,1-b][1,3]thiazol-2-yl]phenyl}methyl)pyrrolidine-1-carboxylate (146-5) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 5; Boc Deprotection; General Procedure II in STEP 6. MS: m/z: Calc'd for C₂₅H₂₂F₄N₄O₃S [M+H]⁺535. found 535. ¹H NMR (400 MHz, Methanol-d₄) δ 8.21 (s, 1H), 8.19-8.15 (m, 1H), 7.65-7.58 (m, 2H), 7.41 (dd, J=14.1, 6.9 Hz, 3H), 7.17 (d, J=7.6 Hz, 1H), 7.13-7.07 (m, 1H), 7.08-7.01 (m, 1H), 4.96 (d, J=3.6 Hz, 1H), 4.49-4.19 (m, 4H), 3.62 (dd, J=12.6, 4.3 Hz, 1H), 3.27 (d, J=12.3 Hz, 1H), 3.25-3.09 (m, 2H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min; Gradient: 23% B to 53% B in 10 min; Wave Length: 254 nm/220 nm; RT1(min): 9.5.

### Synthetic Procedure of (5S,6S,7R)-7-{[4-(1,3-oxazol-5-yl)phenyl]methyl}-3-oxo-4-oxa-1-azabicyclo[3.2.1]octan-6-yl N-[(3-fluorophenyl)methyl]carbamate (V192883)

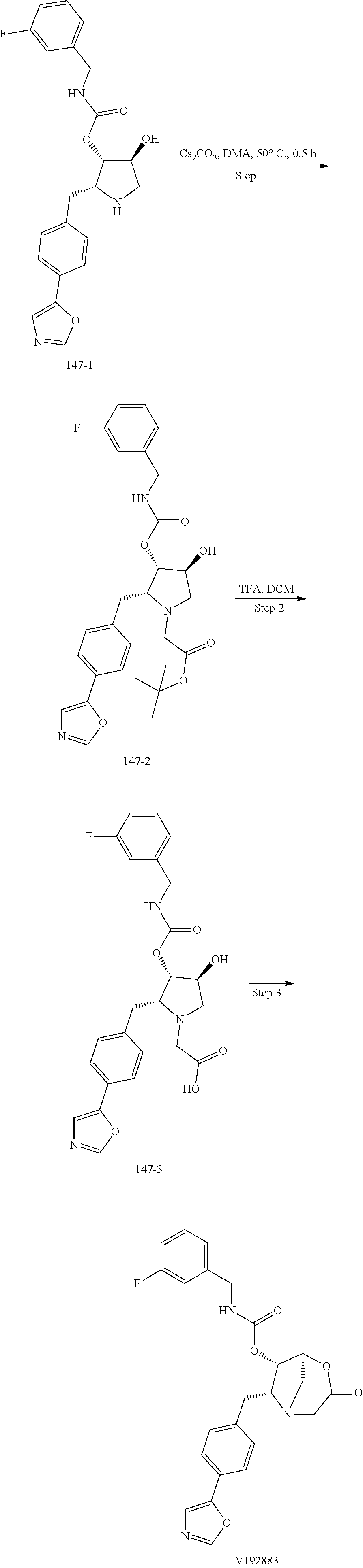 [SMILES: O=C(NCc1cccc(F)c1)O[C@@H]1[C@@H](O)CN[C@@H]1Cc1ccc(-c2cnco2)cc1 ; CC(C)(C)OC(=O)CN1C[C@H](O)[C@@H](OC(=O)NCc2cccc(F)c2)[C@H]1Cc1ccc(-c2cnco2)cc1 ; O=C(O)CN1C[C@H](O)[C@@H](OC(=O)NCc2cccc(F)c2)[C@H]1Cc1ccc(-c2cnco2)cc1 ; O=C1CN2C[C@H](O1)[C@@H](OC(=O)NCc1cccc(F)c1)[C@H]2Cc1ccc(-c2cnco2)cc1]

Step 1: To a stirred mixture of tert-butyl 2-bromoacetate (104.3 mg, 0.53 mmol, 1.1 equiv) and (2R,3S,4S)-4-hydroxy-2-{[4-(1,3-oxazol-5-yl)phenyl]methyl}pyrrolidin-3-yl N-[(3-fluorophenyl)methyl]carbamate (200 mg, 0.48 mmol, 1.00 equiv.) in DMF (20 mL) was added Cs₂CO₃ (475.1 mg, 1.45 mmol, 3 equiv.) at 0° C. under nitrogen atmosphere. The resulting mixture was stirred at 50° C. for 0.5 h under nitrogen atmosphere. The mixture was filtered and the filtrate was purified by reversed-phase flash chromatography to afford tert-butyl 2-[(2R,3S,4S)-3-({[(3-fluorophenyl)methyl]carbamoyl}oxy)-4-hydroxy-2-{[4-(1,3-oxazol-5-yl)phenyl]methyl}pyrroli-din-1-yl]acetate (150 mg, 58.7% yield, 95% purity) as a white solid. MS: m/z: Calc'd for C₂₈H₃₂FN₃O₆ [M+H]⁺ 526. found 526.

Step 2: The compound was prepared in 97% yield as a yellow oil according to Boc Deprotection; General Procedure II. MS: m/z: Calc'd for C₂₄H₂₄FN₃O₆ [M+H]⁺470. found 470.

Step 3: A solution of (2-methyl-6-nitrophenyl)carbonyl 2-methyl-6-nitrobenzoate (70 mg, 0.21 mmol, 1.2 equiv.) in DCM (100 mL) and DMF (5 mL) was treated with DMAP (62.5 mg, 0.51 mmol, 3 equiv.) at room temperature and the above solution was stirred at room temperature for 10 min under nitrogen atmosphere followed by the addition of [(2R,3S,4S)-3-({[(3-fluorophenyl)methyl]carbamoyl}oxy)-4-hydroxy-2-{[4-(1,3-oxazol-5-yl)phenyl]methyl}pyrroli-din-1-yl]acetic acid (80 mg, 0.17 mmol, 1 equiv) dropwise at room temperature. The mixture was stirred at room temperature for overnight. The resulting mixture was concentrated under reduced pressure. The residue was purified by Prep-HPLC to afford the title compound (20.6 mg, 21% yield, 97% purity) as a white solid. MS: m/z: Calc'd for C₂₄H₂₂FN₃O₅ [M+H]⁺452. found 452. ¹H NMR (400 MHz, Methanol-d₄) δ 8.26 (s, 1H), 7.65-7.58 (m, 2H), 7.47 (s, 1H), 7.41 (d, J=8.2 Hz, 2H), 7.34-7.22 (m, 1H), 7.10-6.91 (m, 3H), 5.57 (d, J=6.6 Hz, 1H), 4.97 (s, 1H), 4.40-4.16 (m, 3H), 4.08 (d, J=18.3 Hz, 1H), 3.94-3.81 (m, 1H), 3.56 (d, J=13.3 Hz, 1H), 3.46-3.37 (m, 1H), 3.08-2.90 (m, 2H).

Prep-HPLC-conditions: Column: Xselect CSH Prep C18 Column, 30*250 mm, 10 μm; Mobile Phase A: Water (0.05% TFA), Mobile Phase B: ACN; Flow rate: 60 mL/min mL/min; Gradient: 26% B to 56% B in 10 min; Wave Length: 254 nm/220 nm; RT1(min): 8.5.

### Synthetic Procedure of (2R,3S,4S)-2-{[3-fluoro-4-(1,3-oxazol-5-yl)phenyl]methyl}-4-hydroxypyrrolidin-3-yl N-{2-[(2S)-pyrrolidin-2-yl]ethyl}carbamate (V192903)

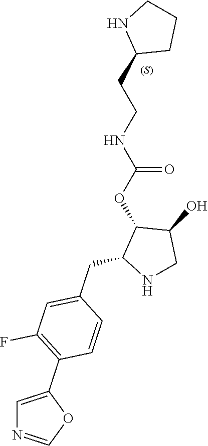

The title compound was prepared in 23.8% overall yield as a light yellow semi-solid according to Substitution reaction for synthesis of Carbamate; General Procedure I using tert-butyl (2R,3S,4S)-4-[(tert-butyldimethylsilyl)oxy]-2-{[3-fluoro-4-(1,3-oxazol-5-yl)phenyl]methyl}-3-[(4-nitrophenoxycarbonyl)oxy]pyrrolidine-1-carboxylate (144-8) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), tert-butyl (2S)-2-(2-aminoethyl)pyrrolidine-1-carboxylate in STEP 1; TBS Deprotection; General Procedure VII in STEP 2; Boc Deprotection; General Procedure II in STEP 3. MS: m/z: Calc'd for C₂₁H₂₇FN₄O₄[M+H]⁺419. Found, 419. ¹H NMR (400 MHz, Methanol-d₄) δ 8.35 (s, 1H), 7.90-7.82 (m, 1H), 7.52 (d, J=3.7 Hz, 1H), 7.36-7.27 (m, 2H), 5.04-4.98 (m, 1H), 4.41 (d, J=4.1 Hz, 1H), 4.31 (d, J=10.1 Hz, 1H), 3.65-3.50 (m, 2H), 3.32-3.10 (m, 7H), 2.35-2.24 (m, 1H), 2.13-1.87 (m, 4H), 1.81-1.65 (m, 1H), 1.31 (s, 2H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min; Gradient: 2% B to 23% B in 10 min; Wave Length: 254 nm/220 nm; RT1(min): 8.5.

### Synthetic Procedure of (2R,3S,4S)-4-hydroxy-2-(4-(thiazol-5-yl)benzyl)pyrrolidin-3-yl isopentylcarbamate (V193648)

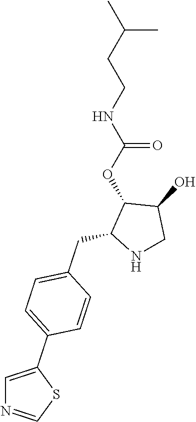 [SMILES: CC(C)CCNC(=O)O[C@@H]1[C@@H](O)CN[C@@H]1Cc1ccc(-c2cncs2)cc1]

The title compound was prepared in 16.6% overall yield as an off-white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-thiazol-5-yl)phenyl]methyl}pyrrolidine-1-carboxylate (Int-12) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z: Calc'd for C₂₀H₂₇N₃O₃S [M+H]⁺390. Found, 390. ¹H NMR (400 MHz, MeOD) δ 8.99 (s, 1H), 8.19 (s, 1H), 7.69 (d, J=7.8 Hz, 2H), 7.42 (d, J=7.9 Hz, 2H), 4.97 (d, J=3.5 Hz, 1H), 4.40 (d, J=4.2 Hz, 1H), 4.33-4.21 (m, 1H), 3.60 (dd, J=12.6, 4.3 Hz, 1H), 3.27-3.06 (m, 5H), 1.75-1.61 (m, 1H), 1.51-1.41 (m, 2H), 0.97 (d, J=6.6 Hz, 6H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min; Gradient (B %): 15% B to 45% B in 10 min; Wave Length: 254 nm/220 nm; RT1(min): 8.47.

### Synthetic Procedure of (2R,3S,4S)-4-hydroxy-2-(4-(thiazol-5-yl)benzyl)pyrrolidin-3-yl (2-(isoxazol-4-yl)ethyl)carbamate (V193647)

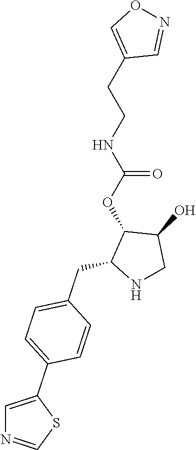 [SMILES: O=C(NCCc1cnoc1)O[C@@H]1[C@@H](O)CN[C@@H]1Cc1ccc(-c2cncs2)cc1]

The title compound was prepared in 17.7% overall yield as an off-white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-thiazol-5-yl)phenyl]methyl}pyrrolidine-1-carboxylate (Int-12) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z: Calc'd for C₂₀H₂₂N₄O₄S [M+H]⁺415. Found, 415. ¹H NMR (400 MHz, MeOD) δ 9.00 (s, 1H), 8.56 (s, 1H), 8.40 (s, 1H), 8.20 (s, 1H), 7.69 (d, J=7.9 Hz, 2H), 7.39 (d, J=7.9 Hz, 2H), 4.95 (d, J=3.4 Hz, 1H), 4.37 (d, J=4.1 Hz, 1H), 4.28-4.19 (m, 1H), 3.58 (dd, J=12.8, 4.2 Hz, 1H), 3.47-3.37 (m, 2H), 3.27-3.22 (m, 1H), 3.21-3.14 (m, 1H), 3.05 (dd, J=14.1, 8.3 Hz, 1H), 2.80-2.72 (m, 2H).

Prep-HPLC-conditions: Column: Xselect CSH Prep Fluoro-Phenyl Column, 30*150 mm, 5 μm; Mobile Phase A: Water (0.05% TFA), Mobile Phase B: ACN; Flow rate: 60 mL/min; Gradient (B %): 3% B to 28% B in 10 min; Wave Length: 254 nm/220 nm; RT1(min): 8.4.

### Synthetic Procedure of (2R,3S,4S)-4-hydroxy-2-(4-(thiazol-5-yl)benzyl)pyrrolidin-3-yl (2-(1H-pyrazol-4-yl)ethyl)carbamate (V193646)

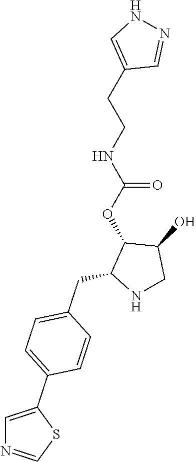 [SMILES: O=C(NCCc1cn[nH]c1)O[C@@H]1[C@@H](O)CN[C@@H]1Cc1ccc(-c2cncs2)cc1]

The title compound was prepared in 34.2% overall yield as an off-white solid according to Substitution reaction for synthesis of Carbamate; General Procedure I using tert-butyl (2R,3S,4S)-4-[(tert-butoxycarbonyl)oxy]-3-[(4-nitrophenoxycarbonyl)oxy]-2-{[4-(1,3-thiazol-5-yl)phenyl]methyl}pyrrolidine-1-carboxylate (Int-12) instead of tert-butyl (2R,3S,4S)-4-[(tert-butoxycarbonyl)oxy]-2-[(4-methoxyphenyl)methyl]-3-[(4-nitrophenoxycarbonyl)oxy]pyrrolidine-1-carboxylate (Int-2), 1-(3-fluorophenyl)methanamine in STEP 1; Boc Deprotection; General Procedure II in STEP 2. MS: m/z: Calc'd for C₂₀H₂₃N₅O₃S [M+H]⁺414. Found, 414. ¹H NMR (400 MHz, MeOD) δ 9.01 (s, 1H), 8.20 (s, 1H), 7.86-7.57 (m, 4H), 7.39 (d, J=8.0 Hz, 2H), 4.96 (d, J=3.5 Hz, 1H), 4.37 (d, J=4.2 Hz, 1H), 4.27-4.18 (m, 1H), 3.59 (dd, J=12.6, 4.3 Hz, 1H), 3.48-3.36 (m, 2H), 3.24 (d, J=12.8 Hz, 1H), 3.21-3.02 (m, 2H), 2.83-2.75 (m, 2H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min; Gradient (B %): 2% B to 31% B in 10 min; Wave Length: 254 nm/220 nm; RT1(min): 9.47.

### Synthetic Procedure of (2R,3S,4S)-4-hydroxy-2-(4-(1-(7-hydroxy-2-oxo-2H-chromen-3-yl)-1H-1,2,3-triazol-4-yl)benzyl)pyrrolidin-3-yl methylcarbamate (V193432)

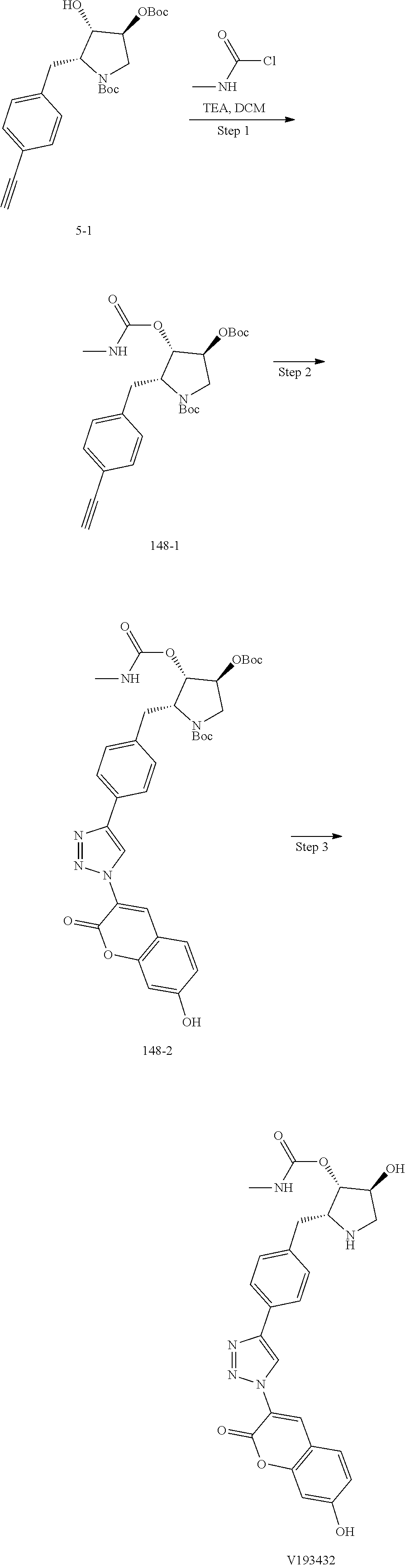

Step 1: To a stirred solution of tert-butyl (2R,3S,4S)-4-[(tert-butoxycarbonyl)oxy]-2-[(4-ethynylphenyl)methyl]-3-hydroxypyrrolidine-1-carboxylate (5-1, 190 mg, 0.46 mmol, 1 equiv) and N-methylcarbamoyl chloride (63.8 mg, 0.68 mmol, 1.5 equiv) in DCM (5 mL) was added Et₃N (138.2 mg, 1.37 mmol, 3 equiv) and DMAP (55.6 mg, 0.46 mmol, 1 equiv) at 0° C. The resulting mixture was stirred at room temperature for 2 h under nitrogen atmosphere. Upon completion. The reaction mixture was quenched with water and extracted with EA. The extracts were washed with brine, dried over anhydrous sodium sulfate, filtered and concentrated. The residue was purified by reversed column chromatography to obtain tert-butyl (2R,3S,4S)-4-[(tert-butoxycarbonyl)oxy]-2-[(4-ethynylphenyl)methyl]-3-[(methylcarbamoyl)oxy]pyrrolidine-1-carboxylate (116 mg) as a light yellow oil. MS: m/z: Calc'd for C₂₅H₃₄N₂O₇ [M+H−56−56]⁺ 363. found [M+H−56−56]⁺363.

Step 2: To a stirred solution of tert-butyl (2R,3S,4S)-4-[(tert-butoxycarbonyl)oxy]-2-[(4-ethynylphenyl)methyl]-3-[(methylcarbamoyl)oxy]pyrrolidine-1-carboxylate (148-1, 116 mg, 0.24 mmol, 1 equiv) and 3-azido-7-hydroxychromen-2-one (59.6 mg, 0.29 mmol, 1.2 equiv) in methanol (5 mL) was added CuSO₄·5H₂O (61.1 mg, 0.24 mmol, 1 equiv) and Sodium ascorbate (96.9 mg, 0.49 mmol, 2 equiv) at 0° C. The resulting mixture was stirred at room temperature for 3 h under nitrogen atmosphere. Upon completion. The reaction mixture was quenched with water and extracted with EA. The extracts were washed with brine, dried over anhydrous sodium sulfate, filtered and concentrated. The residue was purified by reversed chromatography to obtain tert-butyl (2R,3S,4S)-4-[(tert-butoxycarbonyl)oxy]-2-({4-[1-(7-hydroxy-2-oxochromen-3-yl)-1,2,3-triazol-4-yl]phenyl}methyl)-3-[(methylcarbamoyl)oxy]pyrrolidine-1-carboxylate (143 mg, 86.3% yield, 98% purity) as a light yellow oil. MS: m/z: Calc'd for C₃₄H₃₉N₅O₁₀ [M+H]⁺ 678. found [M+H]⁺678.

Step 3: The compound was prepared in 29.5% yield as a yellow oil according to Boc Deprotection; General Procedure II. MS: m/z: Calc'd for C₂₄H₂₃N₅O₆ [M+H]⁺ 358. found [M+H]⁺358. ¹H NMR (400 MHz, MeOD) δ 8.92 (s, 1H), 8.59 (s, 1H), 7.95 (d, J=8.0 Hz, 2H), 7.70 (d, J=8.6 Hz, 1H), 7.46 (d, J=8.0 Hz, 2H), 6.94 (dd, J=8.5, 2.2 Hz, 1H), 6.87 (d, J=2.2 Hz, 1H), 5.02 (s, 1H), 4.42 (d, J=4.2 Hz, 1H), 4.27 (s, 1H), 3.59 (dd, J=12.6, 4.3 Hz, 1H), 3.29-3.23 (m, 2H), 3.10 (d, J=8.8 Hz, 1H), 2.80 (s, 3H).

Prep-HPLC-conditions: Column: Xselect CSH Prep C18 Column, 30*150 mm, 5 μm; Mobile Phase A: Water (0.05% TFA), Mobile Phase B: ACN; Flow rate: 60 mL/min; Gradient: 4% B to 34% B in 10 min; Wave Length: 254 nm/220 nm; RT1(min): 9.37.

### Synthetic Procedure of (2R,3S,4S)-4-hydroxy-2-(4-(thiazol-5-yl)benzyl)pyrrolidin-3-yl (2-((1r,3R)-3-hydroxycyclobutyl)ethyl)carbamate (V193645)

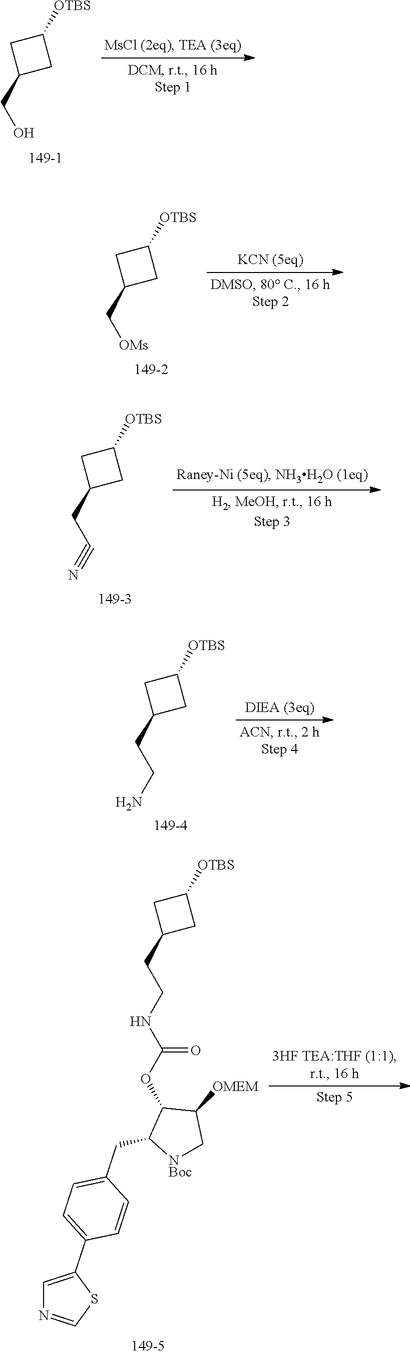

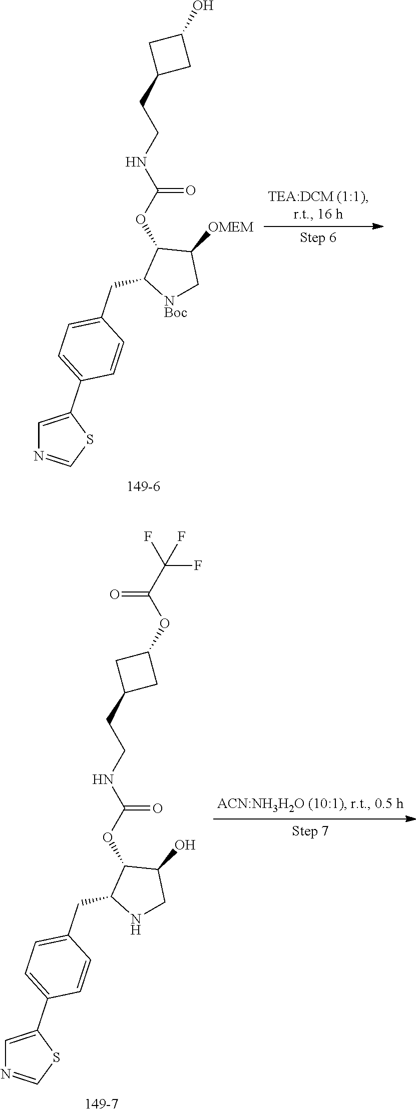

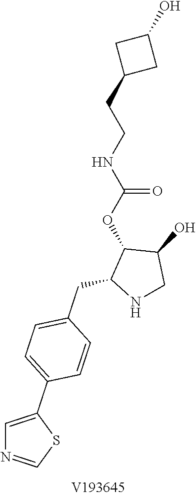 [SMILES: O=C(NCC[C@H]1C[C@H](O)C1)O[C@@H]1[C@@H](O)CN[C@@H]1Cc1ccc(-c2cncs2)cc1]

Step 1: To a stirred solution of [(1r,3r)-3-[(tert-butyldimethylsilyl)oxy]cyclobutyl]methanol (149-1, 250 mg, 1.16 mmol, 1 equiv) in DCM (2 mL) was added Et₃N (351 mg, 3.47 mmol, 3 equiv) and chlorotrimethylsilane (251 mg, 2.31 mmol, 2 equiv) at 0° C. The mixture was stirred for 16 h. The reaction mixture was diluted with DCM (20 mL). The resulting mixture was concentrated under vacuum. The residue was purified by silica gel column chromatography, eluted with (PE/EA=5:1) to afford [(1r,3r)-3-[(tert-butyldimethylsilyl)oxy]cyclobutyl]methyl methanesulfonate (300 mg, 88.2% yield) as a colorless oil. MS: m/z: Calc'd for C₁₂H₂₆O₄SSi [M+H]⁺295. found 295.

Step 2: To a stirred solution of [(1r,3r)-3-[(tert-butyldimethylsilyl)oxy]cyclobutyl]methyl methanesulfonate (149-2, 300 mg, 1.02 mmol, 1 equiv) in DMSO (5 mL) was added KCN (132.7 mg, 2.04 mmol, 2 equiv) at room temperature under nitrogen atmosphere. The mixture was stirred at 80° C. for 16h. Up completion. The reaction mixture was quenched by water and extracted with DCM (3*50 mL). The resulting mixture was concentrated under vacuum. The residue was used in the next step directly without further purification to afford 2-[(1r,3s)-3-[(tert-butyldimethylsilyl)oxy]cyclobutyl]acetonitrile (200 mg crude) as a light yellow oil.

Step 3: To a stirred solution of 2-[(1r,3s)-3-[(tert-butyldimethylsilyl)oxy]cyclobutyl]acetonitrile (149-3, 200 mg, 0.89 mmol, 1 equiv) in methanol (3 mL) and NH₃·H₂O (0.1 mL) was added Raney-Ni (104 mg, 1.77 mmol, 2 equiv) under nitrogen atmosphere. The mixture was hydrogenated at room temperature for 16 h under hydrogen atmosphere using a hydrogen balloon. The reaction progress was monitored by TLC. After completion of reaction, the mixture was filtered through a Celite pad and concentrated under reduced pressure. The crude product was used in the next step directly without further purification to afford 2-[(1r,3s)-3-[(tert-butyldimethylsilyl)oxy]cyclobutyl]ethanamine (110 mg crude) as a light yellow oil. MS: m/z: Calc'd for C₁₂H₂₇NOSi [M+H]⁺230. found 230.

Step 4: To a stirred solution of tert-butyl (2R,3S,4S)-4-[(2-methoxyethoxy)methoxy]-3-[(4-nitrophenoxycarbonyl)oxy]-2-{[4-(1,3-thiazol-5-yl)phenyl]methyl}pyrrolidine-1-carboxylate (50 mg, 0.08 mmol, 1 equiv) in MeCN (2 mL) was added DIEA (30.8 mg, 0.24 mmol, 3 equiv) and 2-[(1r,3s)-3-[(tert-butyldimethylsilyl)oxy]cyclobutyl]ethanamine (149-4, 36.4 mg, 0.16 mmol, 2 equiv) at room temperature. The mixture was stirred for 2 h. The reaction mixture was diluted with ACN (10 mL). The resulting mixture was concentrated under vacuum. The residue was purified by reversed-phase column chromatography (0.05% TFA and MeCN) to afford tert-butyl (2R,3S,4S)-4-[(2-methoxyethoxy)methoxy]-3-[({2-[(1r,3s)-3-[(tert-butyldimethylsilyl)oxy]cyclobutyl]ethyl}carbamoyl)oxy]-2-{[4-(1,3-thiazol-5-yl)phenyl]methyl}pyrrolidine-1-carboxylate (50 mg, 87.5% yield) as a light yellow oil. MS: m/z: Calc'd for C₃₆H₅₇N₃O₈SSi [M+22]⁺742. found 742.

Step 5: To a stirred solution of tert-butyl (2R,3S,4S)-4-[(2-methoxyethoxy)methoxy]-3-[({2-[(1r,3s)-3-[(tert-butyldimethylsilyl)oxy]cyclobutyl]ethyl}carbamoyl)oxy]-2-{[4-(1,3-thiazol-5-yl)phenyl]methyl}pyrrolidine-1-carboxylate (149-5, 50 mg, 0.07 mmol, 1 equiv) in anhydrous THF (1 mL) was added Et₃N·3HF (1 mL) at 0° C. and the resulting mixture was stirred at room temperature for 16 h. The reaction mixture was quenched with water and extracted with EA. The extracts were washed with brine, dried over anhydrous sodium sulfate, filtered and concentrated. The residue was purified by Prep-TLC (0.05% TFA and MeCN) to afford tert-butyl (2R,3S,4S)-4-[(2-methoxyethoxy)methoxy]-3-[({2-[(1r,3s)-3-hydroxycyclobutyl]ethyl}carbamoyl)oxy]-2-{[4-(1,3-thiazol-5-yl)phenyl]methyl}pyrrolidine-1-carboxylate (40 mg, 95.1% yield) as an light yellow oil. MS: m/z: Calc'd for C₃₀H₄₃N₃O₈S [M+H]⁺606. found 606.

Step 6: To a stirred solution of tert-butyl (2R,3S,4S)-4-[(2-methoxyethoxy)methoxy]-3-[({2-[(1r,3s)-3-hydroxycyclobutyl]ethyl}carbamoyl)oxy]-2-{[4-(1,3-thiazol-5-yl)phenyl]methyl}pyrrolidine-1-carboxylate (149-6, 40 mg, 0.07 mmol, 1 equiv) in anhydrous DCM (1.5 mL) was added trifluoroacetic acid (1.5 mL) at 0° C. and the resulting mixture was stirred at room temperature for 12 h. The reaction mixture was quenched with water and extracted with EA. The extracts were washed with brine, dried over anhydrous sodium sulfate, filtered and concentrated. The crude product was used in the next step directly without further purification to afford (1s,3r)-3-{2-[({[(2R,3S,4S)-4-hydroxy-2-{[4-(1,3-thiazol-5-yl)phenyl]methyl}pyrrolidin-3-yl]oxy}carbonyl)amino]ethyl}cyclobutyl 2,2,2-trifluoroacetate (30 mg crude) as a light yellow oil. MS: m/z: Calc'd for C₂₃H₂₆F₃N₃O₅S [M+H]⁺514. found 514.

Step 7: To a stirred solution of (1s,3r)-3-{2-[({[(2R,3S,4S)-4-hydroxy-2-{[4-(1,3-thiazol-5-yl)phenyl]methyl}pyrrolidin-3-yl]oxy}carbonyl)amino]ethyl}cyclobutyl 2,2,2-trifluoroacetate (149-7, 30 mg, 0.058 mmol, 1 equiv) in anhydrous MeCN (2 mL) was added NH₃·H₂O (0.2 mL) at 0° C. and was stirred at room temperature for 1 h. The reaction mixture was concentrated. The residue was purified by Prep-HPLC to obtain (2R,3S,4S)-4-hydroxy-2-{[4-(1,3-thiazol-5-yl)phenyl]methyl}pyrrolidin-3-yl N-{2-[(1r,3s)-3-hydroxycyclobutyl]ethyl}carbamate; formic acid (6.5 mg, 24.0% yield, 98.9% purity) as an off-white semi-solid. MS: m/z: Calc'd for C₂₂H₂₉N₃O₂S [M+H]⁺400. found 400. ¹H NMR (400 MHz, MeOD) δ 8.98 (s, 1H), 8.52 (s, 1H), 8.19 (s, 1H), 7.68 (d, J=8.1 Hz, 2H), 7.41 (d, J=8.1 Hz, 2H), 4.92 (d, J=3.6 Hz, 1H), 4.35 (h, J=5.5 Hz, 2H), 4.23-4.13 (m, 1H), 3.56 (dd, J=12.9, 4.7 Hz, 1H), 3.23-3.00 (m, 5H), 2.34-2.18 (m, 1H), 2.21-1.98 (m, 4H), 1.68-1.64 (m, 2H).

Prep-HPLC conditions: Column: Kinetex EVO C18, 30*150 mm, 5 μm; Mobile Phase A: Water (0.1% FA), Mobile Phase B: MEOH; Flow rate: 25 mL/min mL/min; Gradient (B %): 10% B to 31% B in 12 min; Wave Length: 254 nm/220 nm; RT1(min): 11.82.

### Synthetic Procedure of (2R,3S,4S)-4-hydroxy-2-(4-(thiazol-5-yl)benzyl)pyrrolidin-3-yl (2-((1s,3S)-3-hydroxycyclobutyl)ethyl)carbamate (V193644)

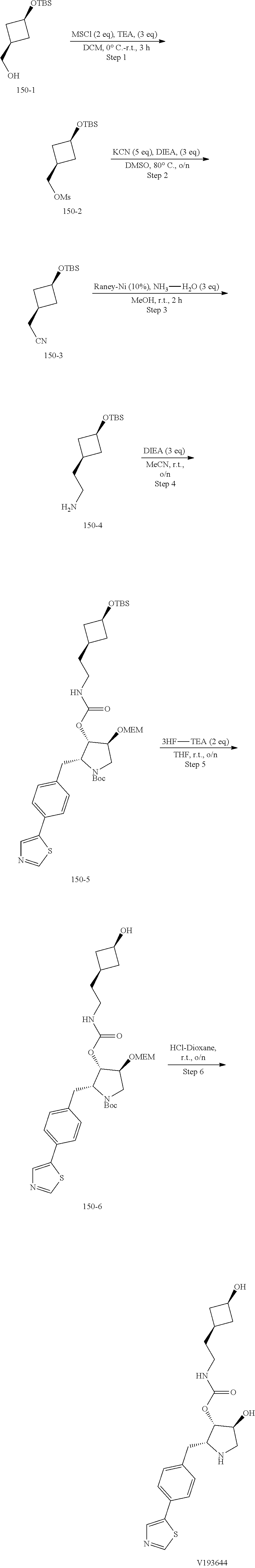

Step 1: To a stirred solution of [(1s,3s)-3-[(tert-butyldimethylsilyl)oxy]cyclobutyl]methanol (150-1, 500 mg, 2.31 mmol, 1 equiv) and Et₃N (701.4 mg, 6.93 mmol, 3 equiv) in DCM (10 mL) was added methanesulfonyl chloride (529.3 mg, 4.62 mmol, 2 equiv) at 0° C. and the mixture was stirred at room temperature for 2 h. The mixture was diluted with water and extracted with EA, the combined organic layers were washed with brine and dried over anhydrous sodium sulfate. After filtration, the filtrate was concentrated under reduced pressure. The resulting residue was purified by column chromatography to obtain [(1s,3s)-3-[(tert-butyldimethylsilyl)oxy]cyclobutyl]methyl methanesulfonate (500 mg, 73.5% yield) as a yellow oil. MS: m/z: Calc'd for C₁₂H₂₆O₄SSi [M+H]⁺295. found 295.

Step 2: To a stirred solution of [(1s,3s)-3-[(tert-butyldimethylsilyl)oxy]cyclobutyl]methyl methanesulfonate (150-2, 500 mg, 1.7 mmol, 1 equiv) in DMSO (12 mL) were added DIEA (877.8 mg, 6.79 mmol, 4 equiv) and KCN (552.8 mg, 8.49 mmol, 5 equiv). The mixture was stirred at 80° C. for 16 h. The mixture was diluted with water and extracted with EA, the combined organic layers were washed with brine and dried over anhydrous sodium sulfate. After filtration, the filtrate was concentrated under reduced pressure to obtain 2-[(1s,3r)-3-[(tert-butyldimethylsilyl)oxy]cyclobutyl]acetonitrile (250 mg) as a yellow oil. The crude product was used in the next step directly without further purification.

Step 3: To a stirred solution of 2-[(1s,3r)-3-[(tert-butyldimethylsilyl)oxy]cyclobutyl]acetonitrile (150-3, 240 mg, 1.1 mmol, 1 equiv) in methanol (8 mL) were added Raney-Ni (125 mg, 2.1 mmol, 2 equiv) and NH₃·H₂O (111.9 mg, 3.2 mmol, 3 equiv) under nitrogen atmosphere. The mixture was hydrogenated at room temperature for 2 h under hydrogen atmosphere using a hydrogen balloon. Then the mixture was filtered through a Celite pad and concentrated to obtain 2-[(1s,3r)-3-[(tert-butyldimethylsilyl)oxy]cyclobutyl]ethanamine (200 mg) as a yellow oil. The crude product was used in the next step directly without further purification. MS: m/z: Calc'd for C₁₂H₂₇NOSi [M+H]⁺230. found 230.

Step 4: To a stirred solution of 2-[(1s,3r)-3-[(tert-butyldimethylsilyl)oxy]cyclobutyl]ethanamine (150-4, 40 mg, 0.17 mmol, 1 equiv) and tert-butyl (2R,3S,4S)-4-[(2-methoxyethoxy)methoxy]-3-[(4-nitrophenoxycarbonyl)oxy]-2-{[4-(1,3-thiazol-5-yl)phenyl]methyl}pyrrolidine-1-carboxylate (13.7 mg, 0.02 mmol, 1 equiv) in MeCN (4 mL) was added DIEA (67.6 mg, 0.52 mmol, 3 equiv) at 0° C. and the mixture was stirred at room temperature for 12 h. The reaction mixture was quenched with water and extracted with EA. The extracts were washed with brine, dried over anhydrous sodium sulfate, filtered and concentrated. The residue was purified by Prep-TLC to obtain tert-butyl (2R,3S,4S)-4-[(2-methoxyethoxy)methoxy]-3-[({2-[(1s,3r)-3-[(tert-butyldimethylsilyl)oxy]cyclobutyl]ethyl}carbamoyl)oxy]-2-{[4-(1,3-thiazol-5-yl)phenyl]methyl}pyrrolidine-1-carboxylate (30 mg, 23.9% yield) as a yellow solid. MS: m/z: Calc'd for C₃₆H₅₇N₃O₈SSi [M+22]⁺742. found 742.

Step 5: To a stirred solution of tert-butyl (2R,3S,4S)-4-[(2-methoxyethoxy)methoxy]-3-[({2-[(1s,3r)-3-[(tert-butyldimethylsilyl)oxy]cyclobutyl]ethyl}carbamoyl)oxy]-2-{[4-(1,3-thiazol-5-yl)phenyl]methyl}pyrrolidine-1-carboxylate (150-5, 30 mg, 0.042 mmol, 1 equiv) in THF (3 mL) was added Et₃N·3HF (33.6 mg, 0.21 mmol, 5 equiv) and the mixture was stirred at room temperature for 5 h. The reaction mixture was quenched with water and extracted with EA. The extracts were washed with brine, dried over anhydrous sodium sulfate, filtered and concentrated. The residue was purified by Prep-TLC to obtain tert-butyl (2R,3S,4S)-4-[(2-methoxyethoxy)methoxy]-3-[({2-[(1s,3r)-3-hydroxycyclobutyl]ethyl}carbamoyl)oxy]-2-{[4-(1,3-thiazol-5-yl)phenyl]methyl}pyrrolidine-1-carboxylate (25 mg, 99.0% yield) as a yellow oil. MS: m/z: Calc'd for C₃₀H₄₃N₃O₈S [M+H]⁺606, found 606.

Step 6: The compound was prepared in 55.8% yield as a yellow oil according to Boc Deprotection; General Procedure II. MS: m/z: Calc'd for C₂₂H₂₉N₃O₂S [M+H]⁺418. found 418. ¹H NMR (400 MHz, MeOD) δ 8.99 (s, 1H), 8.55 (s, 1H), 8.19 (s, 1H), 7.68 (d, J=8.1 Hz, 2H), 7.41 (d, J=8.1 Hz, 2H), 4.92 (d, J=3.6 Hz, 1H), 4.38-4.35 (m, 1H), 4.21-4.14 (m, 1H), 4.11-4.02 (m, 1H), 3.57 (dd, J=12.9, 4.7 Hz, 1H), 3.23-3.00 (m, 5H), 2.55-2.41 (m, 2H), 1.88-1.72 (m, 1H), 1.69-1.62 (m, 2H), 1.58-1.45 (m, 2H).

Prep-HPLC conditions: Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 8% B to 29% B in 17 min; Wave Length: 254 nm/220 nm; RT1(min): 16.55.

### Synthetic Procedure of (2R,3S,4S)-4-hydroxy-2-(4-(thiazol-5-yl)benzyl)pyrrolidin-3-yl (2-((S)-tetrahydrofuran-3-yl)ethyl)carbamate (V193643)

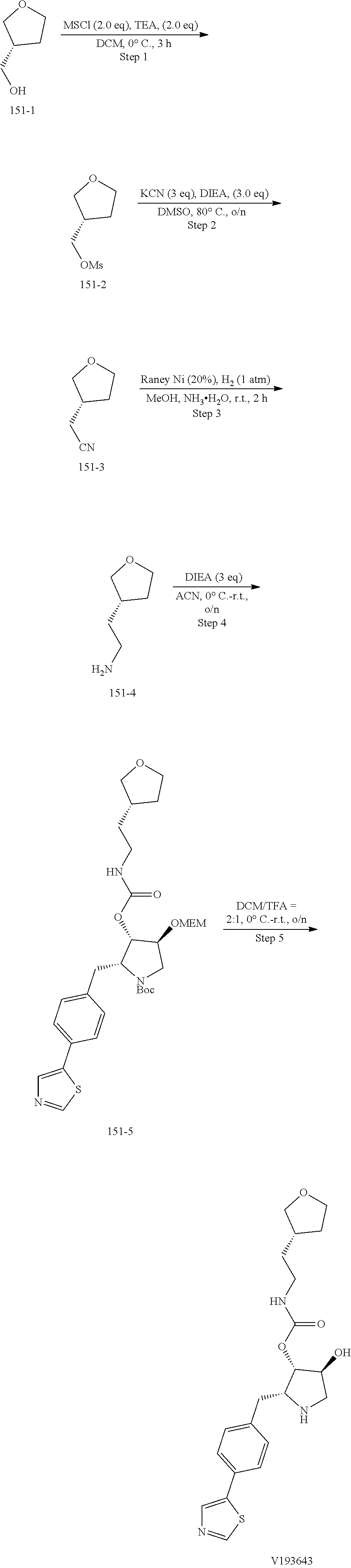

Step 1: To a stirred solution of (3R)-oxolan-3-ylmethanol (151-1, 500 mg, 4.89 mmol, 1 equiv) in DCM (5 mL) was added Et3N (1486 mg, 14.68 mmol, 3 equiv) and methanesulfonyl chloride (1121 mg, 9.79 mmol, 2 equiv) at 0° C. The resulting mixture was stirred at r.t. for 3 h. The reaction mixture was quenched with water and extracted with EA. The extracts were washed with brine, dried over anhydrous sodium sulfate, filtered and concentrated. The residue was purified by silica gel chromatography column (elu with PE/EA=1:1) to obtain (3S)-oxolan-3-ylmethyl methanesulfonate (800 mg, 90.67% yield) as a yellow liquid. MS: m/z: Calc'd for C₆H₁₂O₄S [M+H]⁺181 Found, 181.

Step 2: To a stirred solution of (3S)-oxolan-3-ylmethyl methanesulfonate (151-2, 400 mg, 2.22 mmol, 1 equiv) in DMSO (10 mL) was added DIEA (860 mg, 6.66 mmol, 3 equiv) and KCN (433 mg, 6.66 mmol, 3 equiv). The mixture was stirred at 80° C. for o/n. The mixture was cooled to room temperature and diluted with water and extracted with DCM. The organic layer was washed with water then brine, dried over sodium sulfate, low temperature concentrated. The residue was used in the next step without further purification. MS: m/z: Calc'd for C₆H₉NO.

Step 3: To a stirred solution of 2-[(3S)-oxolan-3-yl]acetonitrile (151-3, 200 mg, 1.79 mmol, 1 equiv) in methanol (5 mL) was added Raney-Ni (40 mg, 0.68 mmol, 0.38 equiv) and NH₃·H₂O (0.1 mL, 0.28 mmol, 0.16 equiv) under N2. The mixture was stirred at r.t. for 3 h under H₂. The solid was filter end out and washed with DCM. The residue was extracted with DCM. The organic layer was dried over sodium sulfate, low temperature concentrated. The crude product was used in the next step without further purification. MS: m/z: Calc'd for C₆H₁₃NO [M+H]⁺116 Found, 116.

Step 4: To a stirred solution of tert-butyl (2R,3S,4S)-4-[(2-methoxyethoxy)methoxy]-3-[(4-nitrophenoxycarbonyl)oxy]-2-{[4-(1,3-thiazol-5-yl)phenyl]methyl}pyrrolidine-1-carboxylate (50 mg, 0.07 mmol, 1 equiv) and 2-[(3S)-oxolan-3-yl]ethanamine (151-4, 50 mg, 0.43 mmol, 5 equiv) in MeCN (3 mL) was added DIEA (33 mg, 0.26 mmol, 3 equiv) at 0° C. The mixture was stirred at r.t. for 1 h. The reaction mixture was quenched with water and extracted with EA. The extracts were washed with brine, dried over anhydrous sodium sulfate, filtered and concentrated. The residue was purified by Prep-TLC to obtain tert-butyl (2R,3S,4S)-4-[(2-methoxyethoxy)methoxy]-3-[({2-[(3S)-oxolan-3-yl]ethyl}carbamoyl)oxy]-2-{[4-(1,3-thiazol-5-yl)phenyl]methyl}pyrrolidine-1-carboxylate (50 mg, 100% yield) as a Colorless oil. MS: m/z: Calc'd for C₃₀H₄₃N₃O₈S [M+H]⁺606 Found, 606.

Step 5: The compound was prepared in 35.7% yield as a yellow oil according to Boc Deprotection; General Procedure II. MS: m/z: Calc'd for C₂₁H₂₇N₃O₄S [M+H]⁺418 Found, 418. ¹H NMR (400 MHz, Methanol-d4) δ 8.99 (s, 1H), 8.20 (s, 1H), 7.70 (d, J=7.8 Hz, 2H), 7.43 (d, J=7.8 Hz, 2H), 4.98 (d, J=3.5 Hz, 1H), 4.41 (d, J=4.1 Hz, 1H), 4.34-4.27 (m, 1H), 4.01-3.84 (m, 2H), 3.77 (t, J=7.7 Hz, 1H), 3.59 (dd, J=12.7, 4.3 Hz, 1H), 3.39 (t, J=7.8 Hz, 1H), 3.29-3.19 (m, 4H), 3.10 (dd, J=14.1, 8.4 Hz, 1H), 2.37-2.08 (m, 2H), 1.73-1.51 (m, 3H).

Prep-HPLC conditions: Column: Xselect CSH Prep Fluoro-Phenyl Column, 30*150 mm, 5 μm; Mobile Phase A: Water (0.05% TFA), Mobile Phase B: ACN; Flow rate: 60 mL/min; Gradient (B %): 3% B to 30% B in 8 min; Wave Length: 254 nm/220 nm; RT1(min): 7.1.

### Synthetic Procedure of (2R,3S,4S)-4-hydroxy-2-(4-(thiazol-5-yl)benzyl)pyrrolidin-3-yl (2-((R)-tetrahydrofuran-3-yl)ethyl)carbamate (V193642)

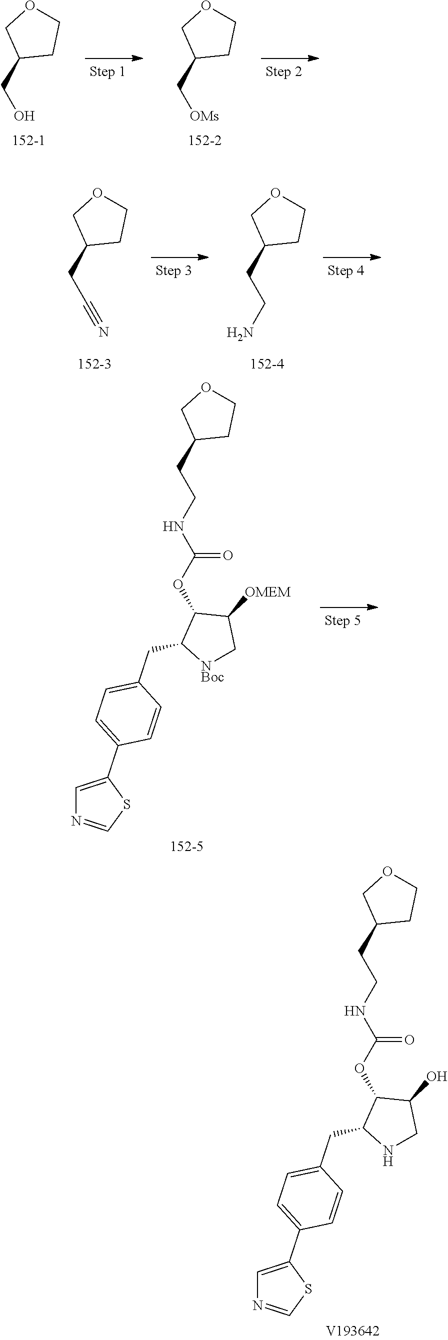

Step 1: To a stirred mixture of (3S)-oxolan-3-ylmethanol (152-1, 500 mg, 4.86 mmol, 1 equiv), pyridine (774.5 mg, 9.79 mmol, 2 equiv) and DMAP (59.8 mg, 0.49 mmol, 0.1 equiv) in DCM (10 mL) was added chlorotrimethylsilane (531.8 mg, 4.86 mmol, 1 equiv) at 0° C. at 0° C. and the resulting mixture was stirred at room temperature for 2 h. The reaction mixture was quenched with water and extracted with EA. The extracts were washed with brine, dried over anhydrous sodium sulfate, filtered and concentrated. The residue was purified by silica gel chromatography column (elu with PE/EA=1:1) to obtain (3R)-oxolan-3-ylmethyl methanesulfonate (500 mg, 56.6% yield) as a yellow oil. MS: m/z: Calc'd for C₆H₁₂O₄S [M+H]⁺ 181. Found, 181.

Step 2: To a stirred mixture of (3R)-oxolan-3-ylmethyl methanesulfonate (152-2, 500 mg, 2.77 mmol, 1 equiv) in DMSO (10 mL) was added KCN (541.9 mg, 8.32 mmol, 3 equiv) The mixture was stirred at 80° C. for o/n. The mixture was cooled to room temperature and diluted with water and extracted with DCM. The organic layer was washed with water then brine, dried over sodium sulfate, low temperature concentrated. The residue was used in the next step without further purification. MS: m/z: Calc'd for C₆H₉NO.

Step 3: To a stirred mixture of 2-[(3R)-oxolan-3-yl]acetonitrile (152-3, 250 mg, 2.24 mmol, 1 equiv) in methanol (10 mL) and NH₃. H₂O (0.1 mL) was added Raney-Ni (660.1 mg, 11.24 mmol, 5 equiv). The mixture was stirred at r.t. for 3 h under H₂. The solid was filter end out and washed with DCM. The residue was extracted with DCM. The organic layer was dried over sodium sulfate, low temperature concentrated. The crude product was used in the next step without further purification. MS: m/z: Calc'd for C₆H₁₃NO [M+H]⁺ 116. Found, 116.

Step 4: To a stirred mixture of 2-[(3R)-oxolan-3-yl]ethanamine (152-4, 29.27 mg, 0.25 mmol, 2 eq) and tert-butyl (2R,3S,4S)-4-[(2-methoxyethoxy)methoxy]-3-[(4-nitrophenoxycarbonyl)oxy]-2-{[4-(1,3-thiazol-5-yl)phenyl]methyl}pyrrolidine-1-carboxylate (80 mg, 0.12 mmol, 1.00 equiv) in MeCN (10 mL) was added DIEA (49.2 mg, 0.38 mmol, 3 equiv) at 0° C. and the resulting mixture was stirred at room temperature for 2 h. The reaction mixture was quenched with water and extracted with EA. The extracts were washed with brine, dried over anhydrous sodium sulfate, filtered and concentrated. The residue was purified by Prep-TLC to obtain tert-butyl (2R,3S,4S)-4-[(2-methoxyethoxy)methoxy]-3-[({2-[(3R)-oxolan-3-yl]ethyl}carbamoyl)oxy]-2-{[4-(1,3-thiazol-5-yl)phenyl]methyl}pyrrolidine-1-carboxylate (50 mg, 64.9% yield, 90% purity) as a yellow solid. MS: m/z: Calc'd for C₃₀H₄₃N₃O₈S [M+H]+ 606. found 606.

Step 5: The compound was prepared in 59.6% yield as a yellow oil according to Boc Deprotection; General Procedure II. MS: m/z: Calc'd for C₂₁H₂₇N₃O₄S [M+H]+ 418. found 418. ¹H NMR (400 MHz, MeOD) δ 8.98 (s, 1H), 8.50 (s, 1H), 8.19 (s, 1H), 7.72-7.65 (m, 2H), 7.45-7.37 (m, 2H), 4.97-4.91 (m, 1H), 4.37 (d, J=4.3 Hz, 1H), 4.22-4.13 (m, 1H), 3.94 (dd, J=8.3, 7.1 Hz, 1H), 3.91-3.84 (m, 1H), 3.76 (q, J=7.9 Hz, 1H), 3.55 (dd, J=12.5, 4.4 Hz, 1H), 3.42-3.34 (m, 1H), 3.30-3.11 (m, 4H), 3.06 (dd, J=14.0, 8.2 Hz, 1H), 2.27-2.24 (m, 1H), 2.13-2.10 (m, 1H), 1.70-1.53 (m, 3H). Prep-HPLC conditions: Kinetex EVO C18, 30*150 mm, 5 μm; Mobile Phase A: Water (0.1% FA), Mobile Phase B: MEOH; Flow rate: 25 mL/min; Gradient (B %): 13% B to 34% B in 12 min; Wave Length: 254 nm/220 nm; RT1(min): 12.49.

### Synthetic Procedure of (2R,3S,4S)-4-hydroxy-2-(4-(thiazol-5-yl)benzyl)pyrrolidin-3-yl (2-((R)-tetrahydrofuran-2-yl)ethyl)carbamate (V193641)

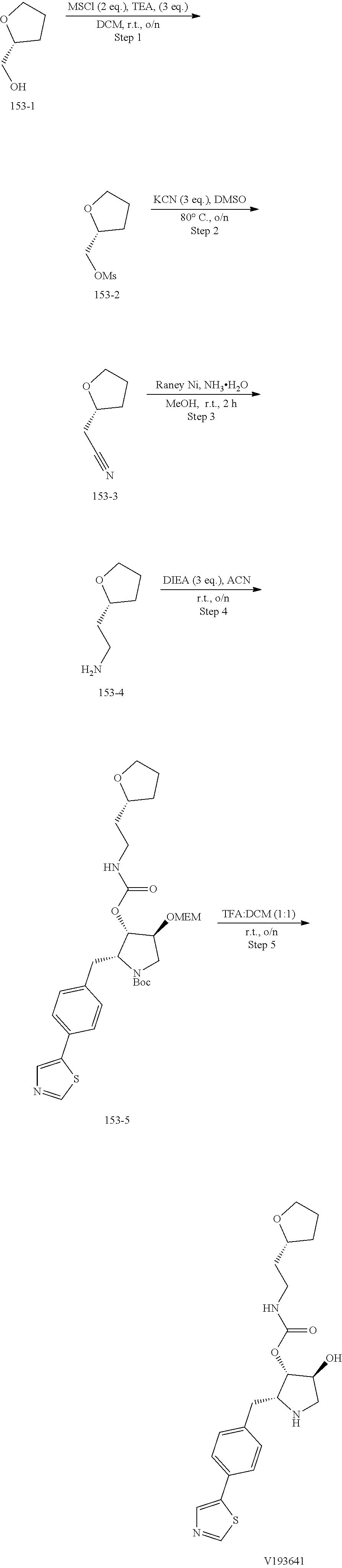

Step 1: To a stirred solution of (2R)-oxolan-2-ylmethanol (153-1, 500 mg, 4.89 mmol, 1 equiv) and TEA (1486.1 mg, 14.68 mmol, 3 equiv) in DCM (8 mL) were added chlorotrimethylsilane (1063.7 mg, 9.79 mmol, 2.00 equiv) at 0° C. The resulting mixture was stirred at room temperature for 2 h. The resulting mixture was concentrated under reduced pressure. The residue was purified by silica gel column chromatography, eluted with PE/EA (1:1) to afford (2R)-oxolan-2-ylmethyl methanesulfonate (800 mg, 90.6% yield) as a light yellow oil. MS: m/z: Calc'd for C₆H₁₂O₄S[M+H]⁺181. Found, 181.

Step 2: To a stirred solution of (2R)-oxolan-2-ylmethyl methanesulfonate (153-2, 400 mg, 2.22 mmol, 1 equiv) in DMSO (7 mL) were added KCN (300 mg, 4.60 mmol, 2.08 equiv) at room temperature. The resulting mixture was stirred at 80° C. for overnight. The resulting mixture was extracted with EtOAc (2×100 mL). The combined organic layers were washed with brine (2×100 mL), dried over anhydrous Na₂SO₄. After filtration, the filtrate was concentrated under reduced pressure to afford 2-[(2R)-oxolan-2-yl]acetonitrile (200 mg, 81.0% yield) as a light yellow semi-solid. The residue was used in the next step without further purification.

Step 3: To a stirred solution of 2-[(2R)-oxolan-2-yl]acetonitrile (153-3, 200 mg, 1.79 mmol, 1 equiv) and NH₃·H₂O (126.1 mg, 3.59 mmol, 2 equiv) in methanol (5 mL) were added Raney-Ni (211.2 mg, 3.59 mmol, 2 equiv). The mixture was stirred at r.t. for 3 h under H₂. The solid was filter end out and washed with DCM. The residue was extracted with DCM. The organic layer was dried over sodium sulfate, low temperature concentrated. The crude product was used in the next step without further purification. MS: m/z: Calc'd for C₆H₁₃NO [M+H]⁺ 116 Found, 116.

Step 4: To a stirred solution of tert-butyl (2R,3S,4S)-4-[(2-methoxyethoxy)methoxy]-3-[(4-nitrophenoxycarbonyl)oxy]-2-{[4-(1,3-thiazol-5-yl)phenyl]methyl}pyrrolidine-1-carboxylate (50 mg, 0.07 mmol, 1 equiv) and 2-[(2R)-oxolan-2-yl]ethanamine (153-4, 18.2 mg, 0.15 mmol, 2 equiv) in ACN (3 mL) were added DIEA (30.7 mg, 0.23 mmol, 3 equiv) at 0° C. The resulting mixture was stirred at room temperature for overnight. The residue was purified by Prep-TLC (EA) to afford tert-butyl (2R,3S,4S)-4-[(2-methoxyethoxy)methoxy]-3-[({2-[(2R)-oxolan-2-yl]ethyl}carbamoyl)oxy]-2-{[4-(1,3-thiazol-5-yl)phenyl]methyl}pyrrolidine-1-carboxylate (15 mg, 31.1% yield) as a light yellow solid. MS: m/z: Calc'd for C₃₀H₄₃N₃O₈S[M+H−100]⁺506. Found, 506.

Step 5: The compound was prepared in 31.9% yield as a yellow oil according to Boc Deprotection; General Procedure II. MS: m/z: Calc'd for C₂₁H₂₇N₃O₄S [M+H]⁺418. found 418. ¹H NMR (400 MHz, Methanol-d₄) δ 9.00 (s, 1H), 8.20 (s, 1H), 7.69 (d, J=7.7 Hz, 2H), 7.43 (d, J=7.9 Hz, 2H), 4.97 (d, J=3.5 Hz, 1H), 4.40 (d, J=4.2 Hz, 1H), 4.25 (s, 1H), 3.90 (dd, J=14.6, 6.8 Hz, 2H), 3.75 (d, J=7.5 Hz, 1H), 3.60 (dd, J=12.7, 4.2 Hz, 1H), 3.24-3.22 (m, 4H), 3.10 (dd, J=14.0, 8.4 Hz, 1H), 2.08-2.06 (m, 1H), 1.98-1.85 (m, 2H), 1.76 (d, J=6.8 Hz, 1H), 1.68 (d, J=7.0 Hz, 1H), 1.61-1.46 (m, 1H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min; Gradient (B %): 7% B to 37% B in 10 min; Wave Length: 254 nm/220 nm; RT1(min): 7.94.

### Synthetic Procedure of (2R,3S,4S)-4-hydroxy-2-(4-(thiazol-5-yl)benzyl)pyrrolidin-3-yl (2-((S)-tetrahydrofuran-2-yl)ethyl)carbamate (V193640)

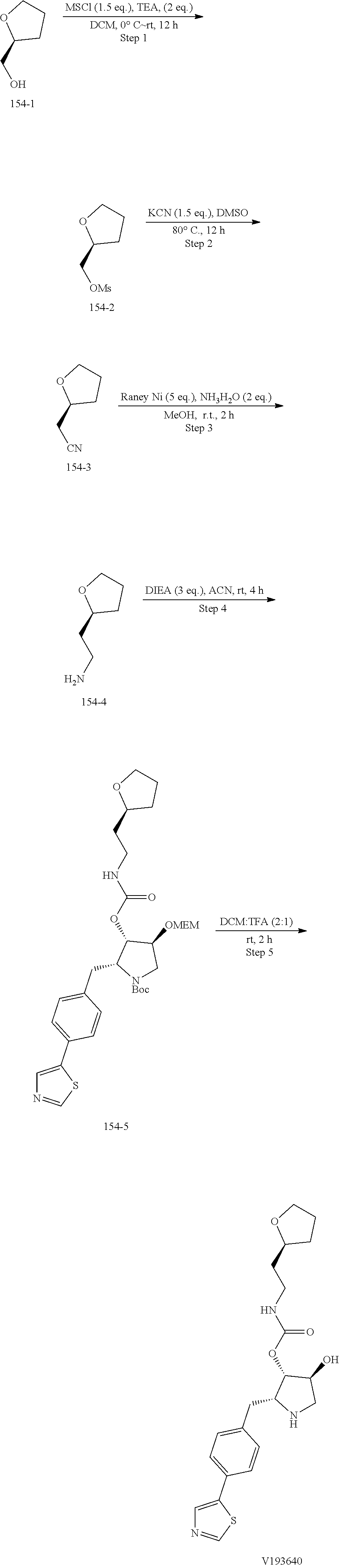

Step 1: To a stirred solution of (2S)-oxolan-2-ylmethanol (154-1, 500 mg, 4.89 mmol, 1 equiv) and Et₃N (644 mg, 6.4 mmol, 1.3 equiv) in DCM (10 mL) was added methanesulfonyl chloride (616.8 mg, 5.39 mmol, 1.1 equiv) at 0° C. and the resulting mixture was stirred at room temperature for 12 h. The reaction mixture was quenched with water and extracted with EA. The extracts were washed with brine, dried over anhydrous sodium sulfate, filtered and concentrated. The residue was purified by Prep-TLC Prep-TLC (PE:EA=1:1) to obtain (2S)-oxolan-2-ylmethyl methanesulfonate (850 mg, 96.3% yield, 98% purity) as a light yellow oil. MS: m/z: Calc'd for C₆H₁₂O₄S [M+H]⁺ 181. found 181.

Step 2: To a stirred solution of (2S)-oxolan-2-ylmethyl methanesulfonate (154-2, 400 mg, 2.22 mmol, 1 equiv) in DMSO (4 mL) was added KCN (216.8 mg, 3.33 mmol, 1.5 equiv) at room temperature. The resulting mixture was stirred at 80° C. for overnight under nitrogen atmosphere. The resulting mixture was extracted with EtOAc (2×100 mL). The combined organic layers were washed with brine (2×100 mL), dried over anhydrous Na₂SO₄. After filtration, the filtrate was concentrated under reduced pressure to afford 2-[(2S)-oxolan-2-yl]acetonitrile (150 mg, 71.0% yield) as a light yellow semi-solid. The residue was used in the next step without further purification.

Step 3: To a stirred solution of 2-[(2S)-oxolan-2-yl]acetonitrile (154-3, 150 mg, 1.35 mmol, 1 equiv) in methanol (6 mL) were added Raney-Ni (396.1 mg, 6.75 mmol, 5 equiv) and NH₃·H₂O (94.6 mg, 2.7 mmol, 2 equiv) The mixture was stirred at r.t. for 3 h under H₂. The solid was filter end out and washed with DCM. The residue was extracted with DCM. The organic layer was dried over sodium sulfate, low temperature concentrated. The crude product was used in the next step without further purification. MS: m/z: Calc'd for C₆H₁₃NO [M+H]⁺ 116 Found, 116.

Step 4: To a stirred solution of tert-butyl (2R,3S,4S)-4-[(2-methoxyethoxy)methoxy]-3-[(4-nitrophenoxycarbonyl)oxy]-2-{[4-(1,3-thiazol-5-yl)phenyl]methyl}pyrrolidine-1-carboxylate (60 mg, 0.1 mmol, 1 equiv) and 2-[(2S)-oxolan-2-yl]ethanamine (154-4, 21.9 mg, 0.19 mmol, 2 equiv) in MeCN (5 mL) was added DIEA (36.9 mg, 0.29 mmol, 3 equiv) at 0° C. and the resulting mixture was stirred at room temperature for 12 h. The reaction mixture was quenched with water and extracted with EA. The extracts were washed with brine, dried over anhydrous sodium sulfate, filtered and concentrated. The residue was purified by Prep-TLC (column, C18 silica gel; mobile phase, MeCN in Water (0.5% TFA), 10% to 50% gradient in 10 min; detector, UV 254 nm) to obtain tert-butyl (2R,3S,4S)-4-[(2-methoxyethoxy)methoxy]-3-[({2-[(2S)-oxolan-2-yl]ethyl}carbamoyl)oxy]-2-{[4-(1,3-thiazol-5-yl)phenyl]methyl}pyrrolidine-1-carboxylate (30 mg) as a light yellow oil. MS: m/z: Calc'd for C₃₀H₄₃N₃O₈S [M+H]⁺ 606. found 606.

Step 5: The compound was prepared in 14.9% yield as a yellow oil according to Boc Deprotection; General Procedure II. MS: m/z: Calc'd for C₂₄H₂₃N₅O₆ [M+H]⁺418. found 418.

¹H NMR (400 MHz, MeOD) δ 8.99 (s, 1H), 8.19 (s, 1H), 7.70 (d, J=7.9 Hz, 2H), 7.42 (d, J=8.2 Hz, 2H), 4.98-4.97 (m, 1H), 4.40 (d, J=4.3 Hz, 1H), 4.29-4.23 (m, 1H), 3.95-3.85 (m, 2H), 3.78-3.73 (m, 1H), 3.63-3.57 (m, 1H), 3.27-3.15 (m, 4H), 3.08 (dd, J=14.1, 8.4 Hz, 1H), 2.13-2.05 (m, 1H), 1.99-1.88 (m, 2H), 1.79-1.71 (m, 2H), 1.59-1.51 (m, 1H).

Prep-HPLC-conditions: Column: XBridge Prep OBD C18 Column, 30*150 mm, 5 μm; Mobile Phase A: Water (0.05% TFA), Mobile Phase B: ACN; Flow rate: 60 mL/min; Gradient (B %): 4% B to 34% B in 10 min; Wave Length: 254 nm/220 nm; RT1(min): 8.69.

### Additional Compounds

The following compounds can be synthesized according to the above procedures:

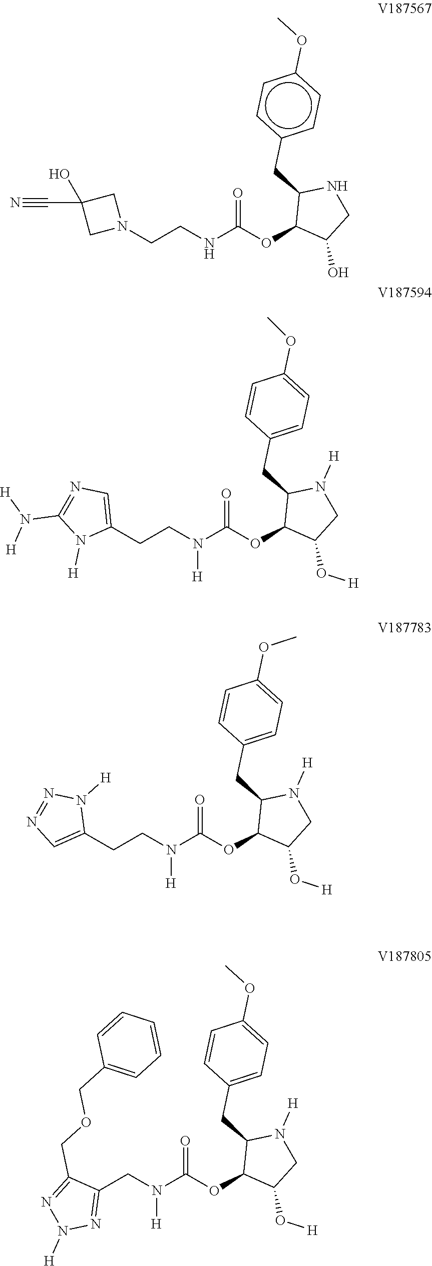

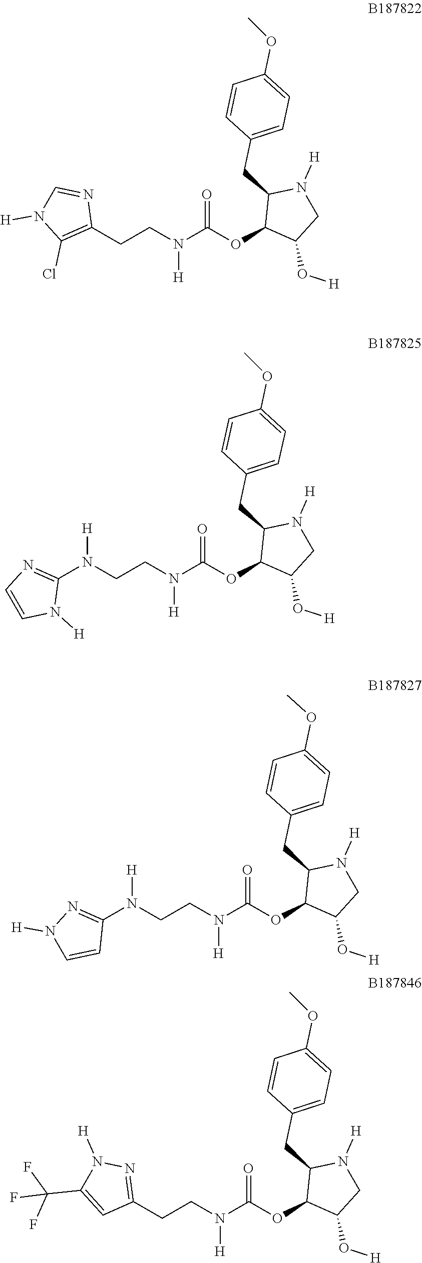

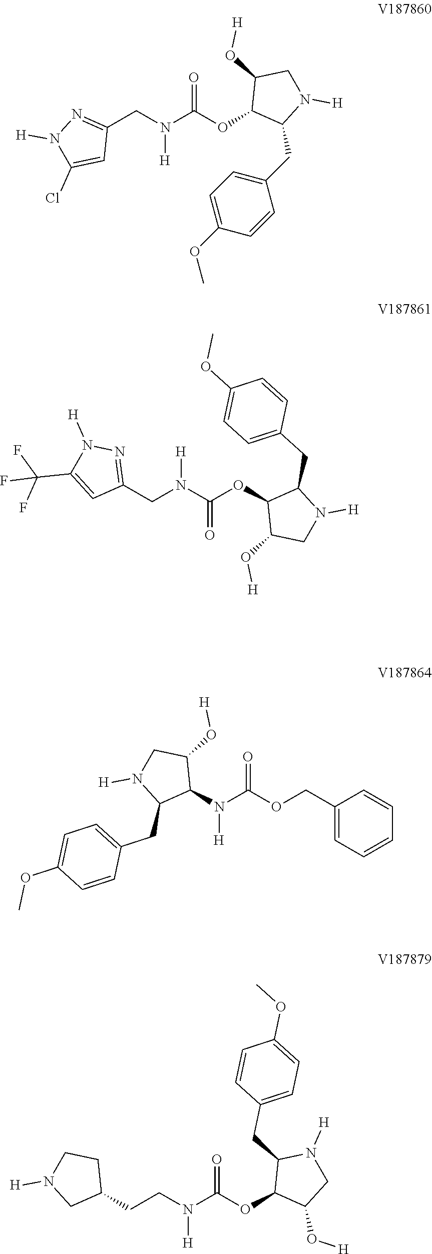

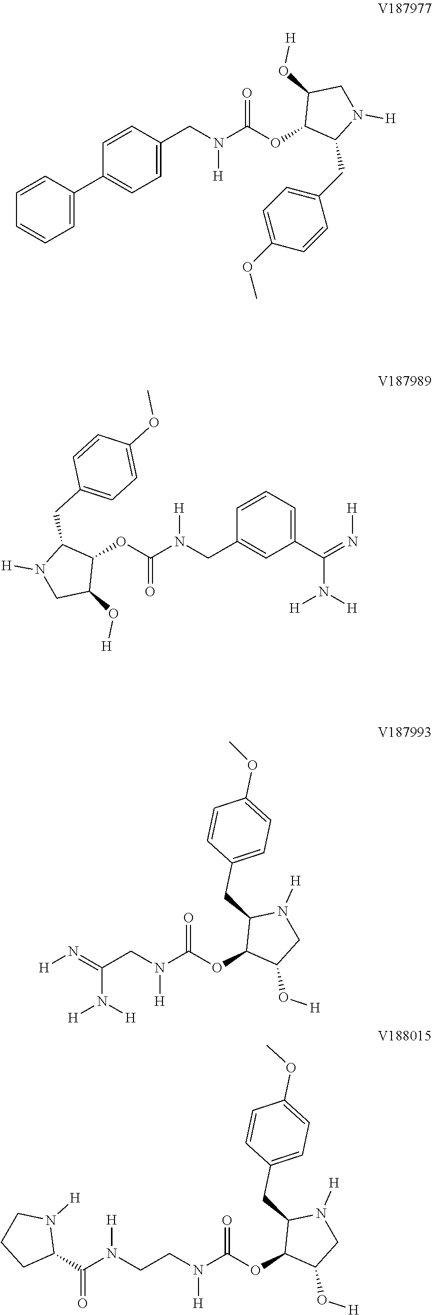 [SMILES: COc1ccc(C[C@H]2NC[C@H](O)[C@H]2OC(=O)NCc2ccc(-c3ccccc3)cc2)cc1 ; [H]/N=C(/N)c1cccc(CNC(=O)O[C@@H]2[C@@H](O)CN[C@@H]2Cc2ccc(OC)cc2)c1 ; [H]/N=C(\N)CNC(=O)O[C@@H]1[C@@H](O)CN[C@@H]1Cc1ccc(OC)cc1 ; COc1ccc(C[C@H]2NC[C@H](O)[C@H]2OC(=O)NCCNC(=O)[C@@H]2CCCN2)cc1]

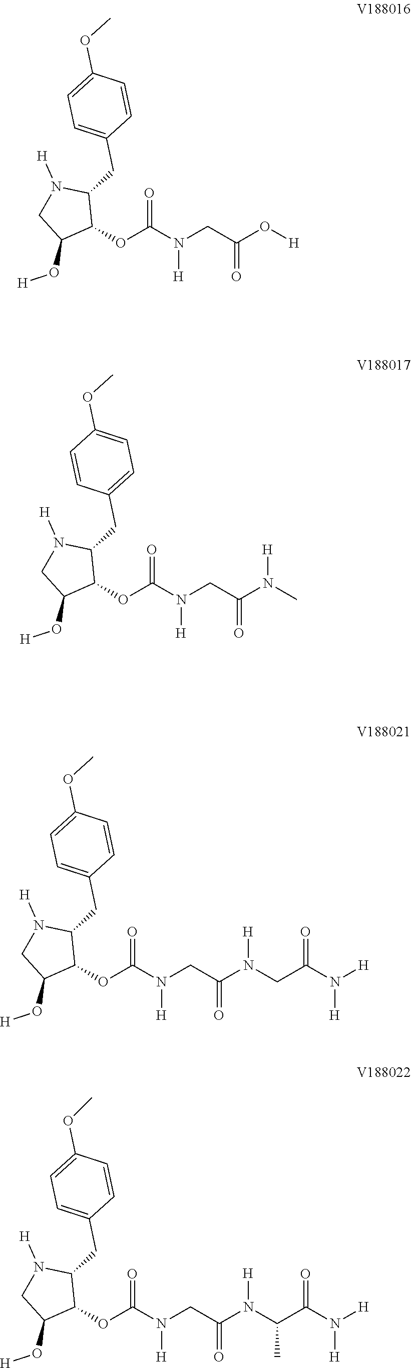

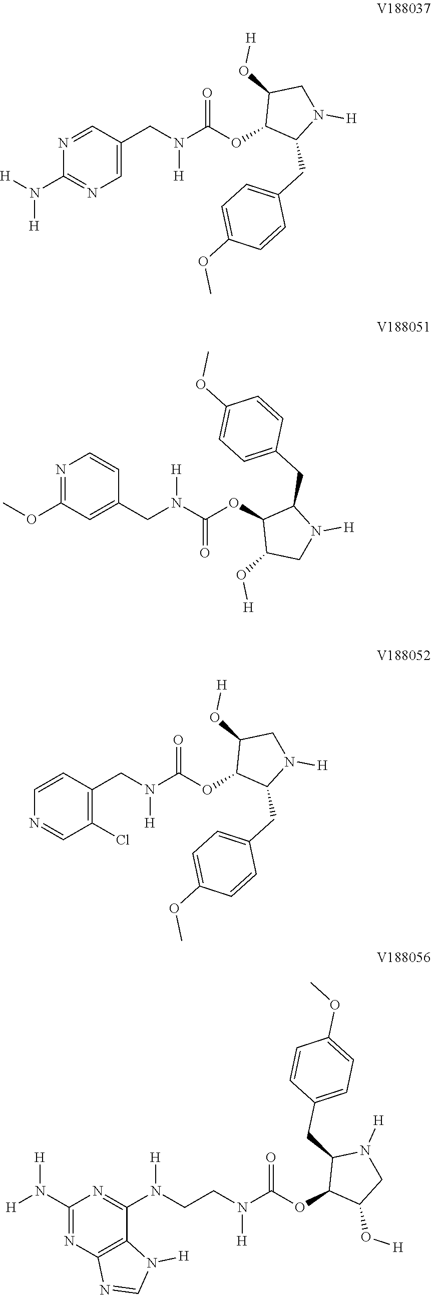 [SMILES: COc1ccc(C[C@H]2NC[C@H](O)[C@H]2OC(=O)NCc2cnc(N)nc2)cc1 ; COc1ccc(C[C@H]2NC[C@H](O)[C@H]2OC(=O)NCc2ccnc(OC)c2)cc1 ; COc1ccc(C[C@H]2NC[C@H](O)[C@H]2OC(=O)NCc2ccncc2Cl)cc1 ; COc1ccc(C[C@H]2NC[C@H](O)[C@H]2OC(=O)NCCNc2nc(N)nc3nc[nH]c23)cc1]

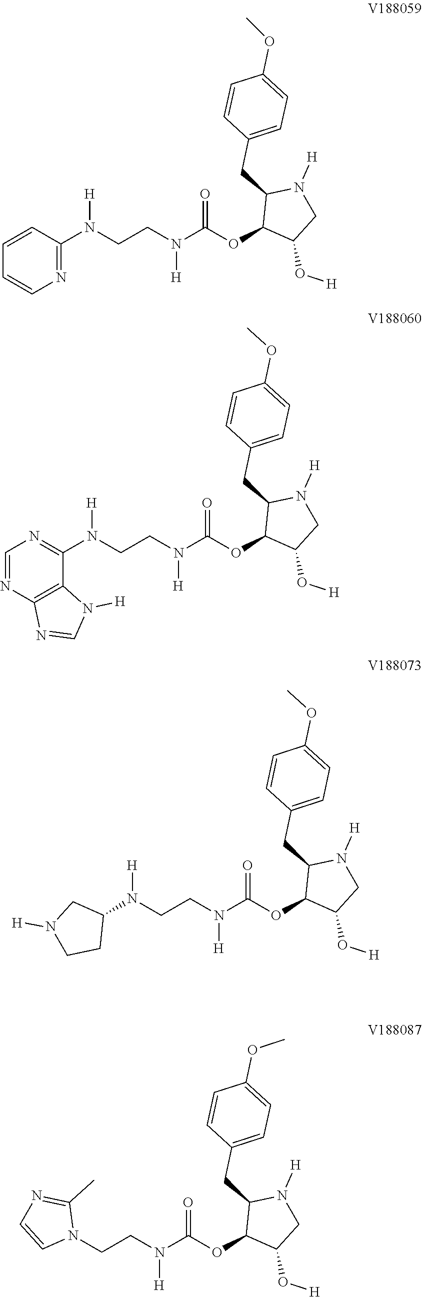 [SMILES: COc1ccc(C[C@H]2NC[C@H](O)[C@H]2OC(=O)NCCNc2ccccn2)cc1 ; COc1ccc(C[C@H]2NC[C@H](O)[C@H]2OC(=O)NCCNc2ncnc3nc[nH]c23)cc1 ; COc1ccc(C[C@H]2NC[C@H](O)[C@H]2OC(=O)NCCN[C@@H]2CCNC2)cc1 ; COc1ccc(C[C@H]2NC[C@H](O)[C@H]2OC(=O)NCCn2ccnc2C)cc1]

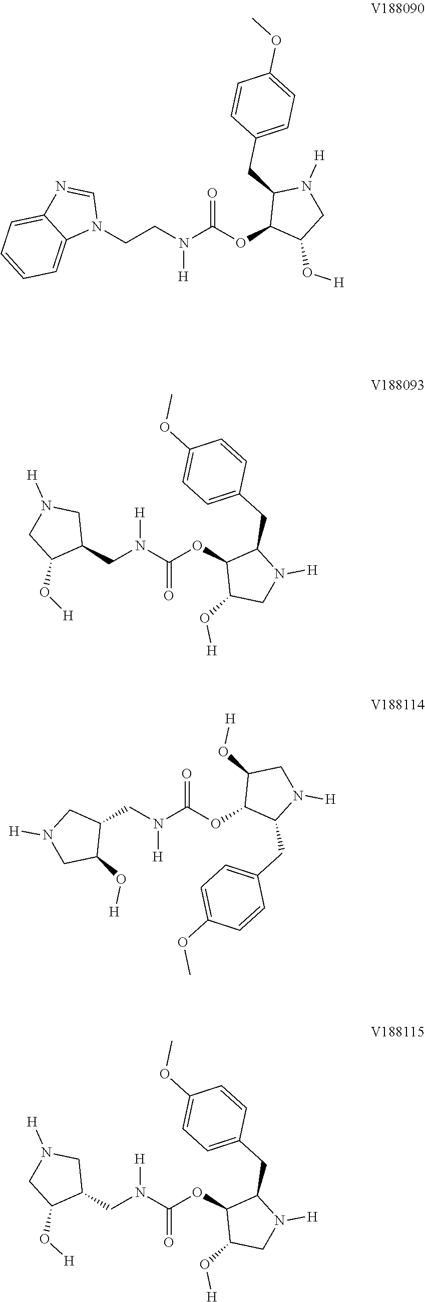

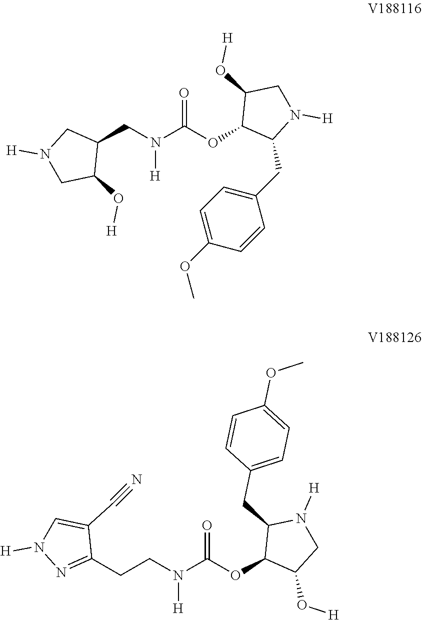 [SMILES: COc1ccc(C[C@H]2NC[C@H](O)[C@H]2OC(=O)NC[C@@H]2CNC[C@@H]2O)cc1 ; COc1ccc(C[C@H]2NC[C@H](O)[C@H]2OC(=O)NCCc2n[nH]cc2C#N)cc1]

### In Vitro Transcription/Translation Data

All reporter constructs have been cloned into the pT7CFE1-CHis plasmid (ThermoFisher #88860) using Gibson assembly procedures (NEB #E2611L). The parental pT7CFE1-CHis plasmid contains an EMCV IRES at the 5′UTR for translation initiation, a multiple-cloning site (MCS) for gene insertion, an optional C-terminal His-epitope tag, and polyA sequence in the 3′UTR to regulate mRNA stability. All reporters are designed to be inserted at the MCS to include a red fluorescent protein (mCherry), P2A self-cleaving peptide which releases the preceding mCherry protein product, and a fusion open reading frame containing a 3×FLAG epitope, a peptide sequence of interest (TEST), and green fluorescent protein (sfGFP).

Construct Plasmid Preparation: All plasmids are isolated from MACH1 cells (ThermoFisher) grown overnight in ampicillin-containing luria broth, and purified using ZymoPure Express Plasmid Midiprep Kit (Zymo Research). Constructs are sequenced verified and diluted to a working concentration of 110 ng/μL.

HeLa S3 Lysate Preparation: HeLa S3 cell pellets are acquired from Ipracell/Ipratech (Belgium) and shipped as 4 mL frozen cell pellets on dry ice for storage at −80° C. until use. The procedure for lysate preparation is as follows. Prepare 1× Lysis Buffer, cold and stored on ice:

| Hela S3 Lysis Buffer (1×) | Vol. for 40 mL of buffer |
|---|---|
| 20 mM Hepes pH 7.4 | 800 μL of 1M stock |
| 10 mM KOAc pH 7.6 | 133.2 μL of 3 M stock |
| 1.8 mM Mg(OAc)2 | 72 μL of 1M stock |
| 1 mM DTT | 40 μL of 1M stock |
| 500 nM ISRIB | 20 μL of 1 mM stock |
| H₂O (cold nuclease free) | 38.936 |

Each preparation takes 8 cell pellets (4 ml packed volume each, approximately 1.0×10⁹ cells per pellet). Thaw cell pellets from −80° C. on ice for 90 mins. Lay pellets horizontally on top of the ice and rotate every 30 mins to ensure even thawing. Once thawed, add 4 mL of cold lysis buffer to each pellet, and using a serological pipet, gently pipet up and down to break up clumps. Incubate resuspended cells in ice for an additional 30 mins (plunged into ice). During 30 min incubation, rinse and RNase-zap (Ambion) glass dounces (use loose pestle “A”), gently dry and place on ice until needed. Prepare RNase-free Eppendorf tubes for spinning and for batch storage. Add batch labels to 1.5-1.7 mL RNase-free microcentrifuge tubes (labeling scheme below). Each batch will have an extra 1.5-1.7 mL RNase-free microcentrifuge tube for 40 μL of lysate, label the top of this tube (labeling scheme below). After 30 min incubation, transfer all 8 mL of cell pellet to dounce. Cell lysis is complete after douncing 100 times. Aliquot cell lysis into 1.5-1.7 mL temporary-microcentrifuge tubes. Place tubes in centrifuge with “fins” out, and spin at 1,200×g for 5 mins. After the first spin use a 1 mL pipet to resuspend each tube and repeat spin. This acts as an additional lysis step, is more vigorous and increases lysate yield. During second spin get liquid N2 and a fresh conical tube on ice. After spin is complete, transfer supernatant (lysate) from microcentrifuge tubes into the 50 mL conical tube and keep on ice. Repeat steps 10-14 for all other cell pellets until completed. Once all cell lysate has been transferred to the 50 mL conical tube pipet to mix so the solution is homogenous. Transfer 1 mL of lysate into batch-labeled microcentrifuge tubes and flash freeze in liquid nitrogen. Be sure to pipet up and down to mix throughout transfer step. Place all tubes in the 80° C. for long-term storage.

Lysate batch quality control: Use IDB-PL005 & IDB-PL055 reporters (highest & lowest signal generators) as controls. Reaction Set-Up included 5 μL Lysate (Previous preparation or new preparation), 1 μL TF accessory proteins, 2 μL TF reaction mix, 1 μL H₂O, and 1 μL of 110 ng/μL plasmid DNA. Set-up 96-well plate as seen below, and incubate reactions at 30° C. for 2 hours:

| | Control lysate reactions | | | Batch #00X Rxns | | |
|---|---|---|---|---|---|---|
| | 1 | 2 | 3 | 4 | 5 | 6 |
| A | Lysate | PL005 | PL055 | Lysate | PL005 | PL055 |
| | Only | | | Only | | |

After 2 hrs, quench the assay using 45 μL of PBS, and transfer 50 μL to a flat-bottom black microplate. Compare fluorescent signals between previous lysate batch and new batch to confirm activity.

In vitro transcription-translation assay setup: Each assay includes a master mix containing lysate and shared components, a 96-well plate containing compound titrations for testing, a 96-well DNA plate with reporter constructs to be tested, 96-well skirted PCR plates, and 96-well flat-bottom, black, fluorescent assay plates. Reaction materials setup: (A) Master mix—15 mL conical tube (3 mL of HeLa S3 Lysate described above; 0.6 mL of ThermoFisher Accessory Proteins (#88882); 1.2 ml of ThermoFisher Reaction Mix (#88882); 0.54 mL of nuclease-free Water); (B) 96-Well Master Mix+Compound Plate (Half-log dilutions (30 μM−0.1 μM) of 4 compounds of interest are prepared in 100% DMSO); 96-Well DNA Plate (Five DNA constructs are plated into single columns within the plate. Row 1 of the column contains nuclease free H2O, all other wells contain 110 ng/μL plasmid DNA). Reactions are started by mixing 155 μL of master mix and 3.5 μL of compound serial dilutions, then 9.1 μL of master mix+compound mixture is aliquoted across 5 96-well plates. Reactions begin with final addition of 2 μL of DNA for a 11.1 μL reaction volume in each well. The entire plate is sealed, and incubated for 2 hrs at 30° C. After 2 hrs, quench the assay using 45 μL of PBS, and transfer 50 μL to a flat-bottom black microplate Quantify the sfGFP and mCherry signals from each well on each plate, taking 3 reads of each plate, and perform data analysis below.

In vitro transcription-translation assay data analysis: Each plate is read three times in channels for both sfGFP and mCherry signals. The average signal across these 3 reads is then calculated for further analysis. Control wells containing no DNA reporter are subtracted from reporter wells in the same column to remove auto-fluorescence of the lysate. Then, the ratio of sfGFP/mCherry is calculated per well, and these ratios are averaged across the triplicate experiments on the same plate. Finally, the ratio of each titration concentration is normalized to the no drug control to create the normalized stalling metric. The error on this measurement is determined by propagating error through the averaging and normalization steps. Values are plotted and fit for IC50 determination for each test sequence and titration of compound.

TABLE E1A
In vitro Transcription-Translation Assay Results

| | BCL-2 IC₅₀ | MYC IC₅₀ | CCND1 IC₅₀ | MCL-1 | ALK IC₅₀ | KRAS G12D |
|---|---|---|---|---|---|---|
| Cmpd | (μM) | (μM) | (μM) | IC₅₀ (μM) | (μM) | IC₅₀ (μM) |
| V0074 | — | A | A | — | A | A |
| V187189 | — | A | — | A | A | A |
| V187190 | A | A | A | A | A | A |
| V187191 | — | C | — | — | A | D |
| V187315 | — | D | — | — | D | D |
| V187327 | — | D | — | — | A | D |
| V187344 | — | D | — | — | D | D |
| V187345 | — | D | — | — | D | D |
| V187381 | — | D | — | — | D | D |
| V187382 | — | D | — | — | D | D |
| V187383 | — | — | — | — | A | D |
| V187384 | — | D | — | — | B | — |
| V187385 | — | B | — | — | A | C |
| V187386 | — | D | — | — | D | D |
| V187387 | — | C | — | — | A | D |
| V187436 | — | D | — | — | D | D |
| V187437 | — | B | — | — | A | C |
| V187439 | — | A | — | — | A | C |
| V187465 | — | A | — | — | A | B |
| V187507 | C | A | C | B | — | B |
| V187508 | — | D | — | — | B | D |
| V187509 | A | A | A | A | — | A |
| V187516 | — | A | — | — | A | B |
| V187518 | — | D | — | — | B | D |
| V187519 | — | — | — | — | — | D |
| V187520 | — | C | — | — | A | D |
| V187521 | — | A | A | A | A | B |
| V187321 | A | A | A | A | A | A |
| V187534 | — | B | — | — | A | — |
| V187536 | — | A | A | A | A | A |
| V187192 | A | A | A | A | A | A |
| V187551 | — | D | — | — | — | D |
| V187552 | — | D | — | — | D | D |
| V187553 | — | D | — | — | D | D |
| V187554 | — | D | — | — | D | D |
| V187565 | — | D | — | — | A | D |
| V187566 | — | D | — | — | B | D |
| V187567 | — | B | C | — | — | C |
| V187568 | — | D | — | — | C | D |
| V187575 | — | C | — | — | A | — |
| V187576 | — | A | — | — | B | B |
| V187577 | — | D | — | — | A | B |
| V187578 | — | D | — | — | C | D |
| V187579 | — | B | — | — | C | — |
| V187580 | — | D | — | — | B | — |
| V187581 | — | C | — | — | — | D |
| V187582 | — | D | — | — | B | D |
| V187583 | — | — | — | — | A | — |
| V187584 | — | — | — | — | A | B |
| V187585 | — | A | A | A | A | — |
| V187586 | — | — | A | A | A | A |
| V187587 | — | A | A | A | A | A |
| V187588 | — | D | — | — | D | — |
| V187589 | — | D | — | — | D | D |
| V187591 | — | A | A | A | A | A |
| V187592 | — | A | — | — | A | A |
| V187593 | A | A | A | A | A | A |
| V187597 | — | D | — | — | D | D |
| V187598 | — | — | — | — | A | A |
| V187620 | — | B | — | — | A | C |
| V187623 | — | D | — | — | C | D |
| V187624 | — | D | — | — | A | D |
| V187625 | — | D | — | — | D | D |
| V187626 | — | D | — | — | C | — |
| V187627 | — | B | — | — | A | C |
| V187628 | — | A | — | — | A | B |
| V187636 | — | A | — | A | A | A |
| V187637 | — | B | — | — | — | C |
| V187638 | — | B | — | — | A | B |
| V187640 | — | D | — | — | — | D |
| V187641 | — | — | — | — | B | — |
| V187642 | — | D | — | — | D | D |
| V187643 | — | — | — | — | C | D |
| V187644 | — | D | — | — | C | D |
| V187652 | — | D | — | — | D | D |
| V187657 | — | B | — | — | A | C |
| V187658 | — | D | — | — | D | D |
| V187659 | — | C | — | — | A | D |
| V187537 | — | A | — | — | A | C |
| V187538 | — | B | — | — | A | D |
| V187539 | — | A | — | — | A | C |
| V187666 | — | — | — | — | D | D |
| V187668 | — | — | — | — | A | B |
| V187679 | — | D | — | — | B | D |
| V187681 | — | D | — | — | D | D |
| V187682 | — | D | — | — | B | D |
| V187684 | — | C | — | — | D | D |
| V187685 | B | A | — | A | — | B |
| V187686 | — | A | B | — | A | B |
| V187688 | — | D | — | — | D | D |
| V187540 | — | D | — | — | B | D |
| V187692 | — | D | — | — | D | — |
| V187700 | — | A | A | A | A | A |
| V187723 | — | B | C | — | A | D |
| V187730 | — | D | — | — | D | D |
| V187739 | — | — | — | — | A | B |
| V187741 | — | A | — | — | A | B |
| V187752 | — | B | — | — | A | — |
| V187753 | — | D | — | — | D | — |
| V187754 | — | — | — | — | A | — |
| V187755 | — | A | A | — | A | — |
| V187767 | — | A | A | A | A | A |
| V187698 | A | A | A | A | A | A |
| V187807 | A | A | — | A | A | A |
| V187812 | B | A | A | A | — | — |
| V187815 | — | A | B | — | A | C |
| V187816 | — | C | D | — | A | D |
| V187819 | — | A | A | A | A | — |
| V187820 | — | B | C | — | A | D |
| V187821 | — | B | D | — | A | D |
| V187818 | — | C | D | — | A | D |
| V187952 | — | C | D | — | A | — |
| V187594 | — | A | A | A | — | — |
| V187599 | — | D | — | B | — | — |
| V187600 | — | B | — | A | — | — |
| V187719 | B | A | A | A | — | — |
| V187783 | A | A | — | A | — | — |
| V187805 | D | — | D | D | — | — |
| V187822 | B | A | B | A | — | — |
| V187823 | B | A | B | A | — | — |
| V187824 | D | D | D | D | — | — |
| V187825 | C | C | B | B | — | — |
| V187826 | C | A | C | B | — | — |
| V187827 | B | A | A | A | — | — |
| V187846 | C | — | D | B | — | — |
| V187847 | C | A | B | A | — | — |
| V187860 | A | A | A | A | — | — |
| V187861 | — | D | C | A | — | — |
| V187862 | — | A | A | A | — | — |
| V187864 | — | D | D | D | — | — |
| V187877 | D | D | D | D | — | — |
| V187878 | B | A | — | A | — | — |
| V187879 | B | A | A | A | — | — |
| V187887 | — | A | A | A | — | — |
| V187916 | A | A | A | A | — | — |
| V187917 | A | A | A | A | — | — |
| V187919 | D | C | D | — | — | — |
| V187928 | D | B | D | B | — | — |
| V187929 | D | D | — | D | — | — |
| V187930 | D | C | C | C | — | — |
| V187940 | A | A | A | A | — | — |
| V187948 | — | A | A | A | — | — |
| V187950 | — | B | A | A | — | — |
| V187977 | D | D | D | D | — | — |
| V187979 | D | D | — | D | — | — |
| V187982 | D | D | D | D | — | — |
| V187987 | A | A | A | A | — | — |
| V187988 | A | A | A | A | — | — |
| V187989 | — | — | A | A | — | — |
| V187993 | — | B | B | B | — | — |
| V188002 | — | C | A | A | — | — |
| V188015 | D | — | B | B | — | — |
| V188016 | D | D | D | D | — | — |
| V188017 | B | — | B | A | — | — |
| V188021 | C | C | C | B | — | — |
| V188022 | C | B | D | A | — | — |
| V188037 | — | A | A | A | — | — |
| V188051 | A | A | A | A | — | — |
| V188052 | B | A | — | A | — | — |
| V188053 | — | A | A | A | — | — |
| V188056 | D | C | D | D | — | — |
| V188058 | — | D | D | D | — | — |
| V188059 | — | B | B | B | — | — |
| V188060 | D | B | C | D | — | — |
| V188073 | — | B | C | B | — | — |
| V188087 | — | B | C | A | — | — |
| V188090 | — | D | D | — | — | — |
| V188093 | — | C | D | — | — | — |
| V188106 | — | A | A | A | — | — |
| V188109 | — | B | D | — | — | — |
| V188114 | — | C | D | B | — | — |
| V188115 | — | D | D | B | — | — |
| V188116 | — | C | D | B | — | — |
| V188126 | A | — | A | A | — | — |

* A = 0 to <10; B = 10 to <20; C = 20 to <30; D ≥ >30; Compounds not tested in any of the IVTT assays were excluded from Table E1A

TABLE E1B
In vitro Transcription-Translation Assay Results*

| Compound | MYC IC₅₀ (μM) | Compound | MYC IC₅₀ (μM) |
|---|---|---|---|
| V193642 | A | V193301 | A |
| V193644 | A | V193129 | A |
| V193645 | A | V193060 | A |
| V193641 | A | V193242 | A |
| V193643 | A | V193139 | A |
| V193648 | A | V193138 | A |
| V193646 | A | V193128 | A |
| V193647 | A | V193081 | A |
| V193351 | A | V193080 | A |
| V193432 | A | V193078 | A |
| V193362 | A | V192275 | A |
| V193210 | B | V193092 | A |
| V193302 | A | V193079 | A |
| V193244 | A | V193019 | A |
| V193234 | A | V192991 | A |
| V193180 | A | V192903 | A |
| V193298 | A | V190507 | A |
| V193262 | A | V193071 | A |
| V193215 | A | V193069 | A |
| V193305 | A | V193068 | A |
| V193263 | A | V193066 | A |
| V193243 | A | V193018 | A |
| V193241 | A | V193013 | A |
| V193240 | A | V192276 | A |
| V193235 | A | V193043 | A |
| V193219 | A | V193012 | A |
| V193218 | A | V193010 | A |
| V193165 | A | V193008 | A |
| V193142 | A | V192915 | A |
| V193040 | A | V192395 | A |
| V193039 | A | V192383 | A |
| V192992 | A | V192433 | A |
| V192990 | A | V192410 | A |
| V192943 | A | V192359 | A |
| V192938 | A | V192310 | A |
| V192912 | A | V192110 | A |
| V192910 | A | V190542 | A |
| V192896 | A | V192392 | A |
| V192894 | A | V192274 | A |
| V192862 | A | V192260 | A |
| V192861 | A | V192129 | A |
| V192860 | A | V192289 | A |
| V190508 | A | V192288 | A |
| V192234 | A | V192286 | A |
| V192169 | A | V192136 | A |
| V192131 | A | V192133 | A |
| V193011 | A | V192127 | A |
| V192930 | A | V192118 | A |
| V192929 | A | V192331 | A |
| V192917 | A | V192324 | A |
| V192916 | A | V192261 | A |
| V192905 | A | V192142 | A |
| V192897 | A | V192265 | A |
| V192893 | A | V192264 | A |
| V192847 | A | V192232 | A |
| V192907 | A | V192156 | D |
| V192901 | A | V192154 | A |
| V192874 | A | V192139 | A |
| V192851 | A | V190552 | A |
| V192850 | A | V190444 | D |
| V192849 | A | V192262 | A |
| V192429 | A | V192167 | A |
| V192418 | A | V192160 | A |
| V192398 | A | V191891 | A |
| V192320 | A | V192269 | A |
| V192319 | A | V192116 | A |
| V192868 | A | V192102 | B |
| V192385 | A | V190447 | C |
| V192159 | A | V192114 | D |
| V192442 | A | V192113 | A |
| V192432 | A | V192112 | A |
| V192411 | A | V192109 | A |
| V192103 | A | V192038 | D |
| V192063 | B | V192037 | B |
| V192041 | A | V192036 | A |
| V192073 | A | V191989 | A |
| V192071 | C | V191988 | A |
| V192069 | A | V191970 | D |
| V192068 | A | V191968 | D |
| V192060 | A | V191963 | D |
| V192056 | A | V191945 | A |
| V192045 | B | V191943 | A |
| V192040 | B | V191920 | A |
| V191944 | A | V191962 | D |
| V191921 | A | V191955 | A |
| V192083 | D | V191954 | A |
| V192067 | A | V191948 | B |
| V192066 | A | V191932 | A |
| V192065 | B | V191930 | B |
| V192059 | D | V191906 | A |
| V192057 | A | V191902 | B |
| V192053 | D | V190559 | A |
| V192051 | A | V190547 | A |
| V192049 | A | V190546 | A |
| V192046 | A | V190515 | A |
| V191961 | B | V191915 | A |
| V191903 | A | V191911 | A |
| V190543 | A | V191904 | A |
| V190519 | C | V191885 | A |
| V188209 | D | V190517 | A |
| V192058 | D | V190516 | A |
| V192052 | D | V190514 | A |
| V192050 | B | V190511 | A |
| V192048 | A | V188356 | A |
| V192047 | A | V191886 | A |
| V192043 | D | V190545 | A |
| V192042 | D | V190538 | D |
| V191992 | C | V190520 | A |
| V191971 | B | V190512 | A |
| V191956 | D | V190505 | A |
| V191939 | B | V190449 | A |
| V190526 | B | V190410 | |
| V190446 | A | V188354 | A |
| V190441 | D | V190522 | A |
| V192039 | D | V190521 | C |
| V190518 | B | V188342 | A |
| V190509 | D | V188341 | A |
| V190506 | A | V188311 | A |
| V190459 | A | V188307 | A |
| V190414 | D | V188306 | A |
| V188406 | A | V188299 | A |
| V188405 | A | V188337 | A |
| V188398 | A | V188331 | A |
| V188330 | A | V188327 | A |
| V188329 | A | V188322 | A |
| V188366 | A | V188321 | D |
| V188347 | A | V188318 | A |
| V190415 | D | V188315 | B |
| V188382 | A | V188310 | A |
| V188376 | A | V188298 | A |
| V188365 | A | V188296 | A |
| V188360 | A | V188305 | A |
| V188355 | A | V188304 | A |
| V188339 | A | V188291 | B |
| V188319 | B | V188289 | A |
| V190413 | B | V188288 | B |
| V190411 | B | V188287 | A |
| V190408 | A | V188286 | D |
| V188392 | A | V188276 | B |
| V188384 | A | V188275 | A |
| V188381 | A | V188274 | A |
| V188373 | A | V188273 | D |
| V188361 | A | V188272 | B |
| V188345 | A | V188271 | B |
| V188344 | A | V188270 | A |
| V188340 | A | V188269 | D |
| V188324 | B | V188228 | D |
| V188323 | B | V188227 | C |
| V188316 | A | V188203 | B |
| V188314 | A | V188149 | D |
| V188303 | A | V188080 | A |
| V188297 | A | V187808 | A |
| V188383 | A | V188229 | A |
| V188380 | A | V188226 | A |
| V188379 | A | V188225 | A |
| V188378 | A | V188150 | A |
| V188377 | A | V188144 | A |
| V188348 | A | V188131 | A |
| V188084 | A | V187925 | A |
| V188076 | D | V188188 | D |
| V188071 | B | V188175 | B |
| V188035 | A | V188172 | D |
| V188210 | B | V188167 | D |
| V188206 | A | V188164 | D |
| V188205 | B | V188161 | D |
| V188202 | A | V188160 | C |
| V188171 | B | V188154 | A |
| V188170 | B | V188151 | A |
| V188165 | C | V188148 | A |
| V188162 | D | V188147 | A |
| V188152 | — | V188138 | A |
| V188143 | A | V188135 | B |
| V188108 | D | V188088 | C |
| V188089 | B | V188050 | A |
| V188086 | B | V187944 | B |
| V188085 | B | V188018 | B |
| V188079 | D | V188019 | B |

*A= 0 to <10; B = 10 to <20; C = 20 to <30; D = ≥30; Compounds not tested in any of the IVTT assays were excluded from Table E1B

TABLE E1C
In vitro Transcription-Translation Assay Results*

| | CCND1 | CCNE1 | MCL1 | CCNTB1 | cGAS | STING | JAK1 | NFKB1 |
|---|---|---|---|---|---|---|---|---|
| | IC₅₀ | IC₅₀ | IC₅₀ | IC₅₀ | IC₅₀ | IC₅₀ | IC₅₀ | IC₅₀ |
| Cmpd | (μM) | (μM) | (μM) | (μM) | (μM) | (μM) | (μM) | (μM) |
| V193351 | — | A | A | — | — | — | — | — |
| V193432 | — | — | — | — | — | — | — | — |
| V193362 | — | B | B | — | — | — | — | — |
| V193210 | — | C | B | — | — | — | — | — |
| V193302 | — | A | A | — | — | — | — | — |
| V193244 | — | A | A | — | — | — | — | — |
| V193234 | — | A | A | — | — | — | — | — |
| V193180 | — | A | A | — | — | — | — | — |
| V193298 | — | A | A | — | — | — | — | — |
| V193262 | — | A | A | — | — | — | — | — |
| V193215 | — | A | A | — | — | — | — | — |
| V193305 | — | A | A | — | — | — | — | — |
| V193263 | — | A | A | — | — | — | — | — |
| V193243 | — | A | A | — | — | — | — | — |
| V193241 | — | A | A | — | — | — | — | — |
| V193240 | — | A | A | — | — | — | — | — |
| V193235 | — | A | A | — | — | — | — | — |
| V193219 | — | A | A | — | — | — | — | — |
| V193218 | — | A | A | — | — | — | — | — |
| V193165 | — | A | A | — | — | — | — | — |
| V193142 | — | B | A | — | — | — | — | — |
| V193301 | — | A | A | — | — | — | — | — |
| V193129 | — | A | A | — | — | — | — | — |
| V193060 | — | A | A | — | — | — | — | — |
| V193242 | — | A | A | — | — | — | — | — |
| V193139 | — | A | A | — | — | — | — | — |
| V193138 | — | A | A | — | — | — | — | — |
| V193128 | — | A | A | — | — | — | — | — |
| V193081 | — | A | A | — | — | — | — | — |
| V193080 | — | A | A | — | — | — | — | — |
| V193078 | — | A | A | — | — | — | — | — |
| V192275 | A | — | — | — | — | — | — | A |
| V193092 | — | A | A | — | — | — | — | — |
| V193079 | — | A | A | — | — | — | — | — |
| V193019 | — | A | A | — | — | — | — | — |
| V192991 | — | A | A | — | — | — | — | — |
| V192903 | B | — | — | — | — | — | — | A |
| V190507 | A | — | — | — | — | — | — | A |
| V193071 | — | A | A | — | — | — | — | — |
| V193069 | — | A | A | — | — | — | — | — |
| V193068 | — | A | A | — | — | — | — | — |
| V193066 | — | A | A | — | — | — | — | — |
| V193018 | — | A | A | — | — | — | — | — |
| V193013 | — | A | A | — | — | — | — | — |
| V192276 | A | — | — | — | — | — | — | A |
| V193043 | — | A | A | — | — | — | — | — |
| V193012 | — | A | A | — | — | — | — | — |
| V193010 | — | A | B | — | — | — | — | — |
| V193008 | — | A | A | — | — | — | — | — |
| V192915 | A | — | — | — | — | — | — | A |
| V193040 | — | A | A | — | — | — | — | — |
| V193039 | — | A | A | — | — | — | — | — |
| V192992 | — | A | A | — | — | — | — | — |
| V192990 | — | A | A | — | — | — | — | — |
| V192943 | — | A | A | — | — | — | — | — |
| V192938 | — | A | A | — | — | — | — | — |
| V192912 | A | — | — | — | — | — | — | A |
| V192910 | A | — | — | — | — | — | — | A |
| V192896 | A | — | — | — | — | — | — | A |
| V192894 | A | — | — | — | — | — | — | A |
| V192862 | A | — | — | — | — | — | — | A |
| V192861 | A | — | — | — | — | — | — | A |
| V192860 | A | — | — | — | — | — | — | A |
| V190508 | A | — | — | — | — | — | — | A |
| V192234 | A | — | — | — | — | — | — | A |
| V192169 | A | — | — | — | — | — | — | A |
| V192131 | A | — | — | — | — | — | — | A |
| V193011 | — | A | A | — | — | — | — | — |
| V192930 | — | A | A | — | — | — | — | — |
| V192929 | — | A | A | — | — | — | — | — |
| V192917 | A | — | — | — | — | — | — | A |
| V192916 | A | — | — | — | — | — | — | A |
| V192905 | A | — | — | — | — | — | — | A |
| V192897 | B | — | — | — | — | — | — | A |
| V192893 | A | — | — | — | — | — | — | A |
| V192847 | A | — | — | — | — | — | — | A |
| V192907 | A | — | — | — | — | — | — | A |
| V192901 | A | — | — | — | — | — | — | A |
| V192874 | A | — | — | — | — | — | — | A |
| V192851 | A | — | — | — | — | — | — | A |
| V192850 | A | — | — | — | — | — | — | A |
| V192849 | A | — | — | — | — | — | — | A |
| V192429 | A | — | — | — | — | — | — | A |
| V192418 | A | — | — | — | — | — | — | A |
| V192398 | A | — | — | — | — | — | — | A |
| V192320 | A | — | — | — | — | — | — | A |
| V192319 | A | — | — | — | — | — | — | A |
| V192868 | A | — | — | — | — | — | — | A |
| V192385 | A | — | — | — | — | — | — | A |
| V192159 | A | — | — | — | — | — | — | A |
| V192442 | A | — | — | — | — | — | — | A |
| V192432 | A | — | — | — | — | — | — | A |
| V192411 | A | — | — | — | — | — | — | A |
| V192395 | A | — | — | — | — | — | — | A |
| V192383 | A | — | — | — | — | — | — | A |
| V192433 | A | — | — | — | — | — | — | A |
| V192410 | A | — | — | — | — | — | — | A |
| V192359 | A | — | — | — | — | — | — | A |
| V192310 | B | — | — | — | — | — | — | A |
| V192110 | A | — | — | — | — | — | — | A |
| V190542 | A | — | — | — | — | — | — | A |
| V192392 | A | — | — | — | — | — | — | A |
| V192274 | A | — | — | — | — | — | — | A |
| V192260 | A | — | — | — | — | — | — | A |
| V192129 | A | — | — | — | — | — | — | A |
| V192289 | A | — | — | — | — | — | — | A |
| V192288 | A | — | — | — | — | — | — | A |
| V192286 | A | — | — | — | — | — | — | A |
| V192136 | A | — | — | — | — | — | — | A |
| V192133 | A | — | — | — | — | — | — | A |
| V192127 | A | — | — | — | — | — | — | A |
| V192118 | A | — | — | — | — | — | — | A |
| V192331 | A | — | — | — | — | — | — | A |
| V192324 | A | — | — | — | — | — | — | A |
| V192261 | A | — | — | — | — | — | — | A |
| V192142 | A | — | — | — | — | — | — | A |
| V192265 | A | — | A | A | A | A | A | A |
| V192264 | A | — | A | A | — | — | — | — |
| V192232 | A | — | A | A | — | — | — | — |
| V192156 | D | — | D | D | — | — | — | — |
| V192154 | A | — | A | A | A | A | A | A |
| V192139 | A | — | A | A | — | — | — | — |
| V190552 | A | — | A | A | — | — | — | — |
| V190444 | D | — | D | D | — | — | — | — |
| V192262 | A | — | A | A | — | — | — | — |
| V192167 | A | — | A | A | — | — | — | — |
| V192160 | A | — | A | A | A | A | A | A |
| V191891 | B | — | A | A | — | — | — | — |
| V192269 | A | — | A | A | A | A | A | A |
| V192116 | B | — | A | A | — | — | — | — |
| V192102 | D | — | D | B | — | — | — | — |
| V190447 | D | — | D | B | — | — | — | — |
| V192114 | D | — | D | D | A | D | B | A |
| V192113 | A | — | A | A | — | — | — | — |
| V192112 | A | — | A | A | — | — | — | — |
| V192109 | A | — | A | A | — | — | — | — |
| V192103 | B | — | A | A | — | — | — | — |
| V192063 | C | — | A | A | — | — | — | — |
| V192041 | B | — | A | A | — | — | — | — |
| V192073 | A | — | A | A | — | — | — | — |
| V192071 | D | — | C | C | — | — | — | — |
| V192069 | A | — | A | A | — | — | — | — |
| V192068 | B | — | A | A | — | — | — | — |
| V192060 | C | — | A | A | — | — | — | — |
| V192056 | A | — | A | A | — | — | — | — |
| V192045 | C | — | B | A | — | — | — | — |
| V192040 | C | — | B | A | — | — | — | — |
| V191944 | B | — | A | A | — | — | — | — |
| V191921 | B | — | A | A | — | — | — | — |
| V192083 | D | — | D | D | — | — | — | — |
| V192067 | A | — | A | B | — | — | — | — |
| V192066 | A | — | A | B | — | — | — | — |
| V192065 | D | — | B | B | — | — | — | — |
| V192059 | D | — | D | D | — | — | — | — |
| V192057 | B | — | A | A | — | — | — | — |
| V192053 | D | — | C | B | — | — | — | — |
| V192051 | A | — | A | A | — | — | — | — |
| V192049 | A | — | A | A | A | A | A | A |
| V192046 | B | — | A | A | — | — | — | — |
| V191961 | B | — | A | A | — | — | — | — |
| V191903 | B | — | A | A | — | — | — | — |
| V190543 | D | — | D | C | — | — | — | — |
| V190519 | D | — | D | C | — | — | — | — |
| V188209 | D | — | D | D | — | — | — | — |
| V192058 | D | — | D | C | — | — | — | — |
| V192052 | D | — | D | D | — | — | — | — |
| V192050 | D | — | B | B | — | — | — | — |
| V192048 | A | — | A | A | — | — | — | — |
| V192047 | A | — | A | A | A | A | A | A |
| V192043 | D | — | D | D | — | — | — | — |
| V192042 | D | — | D | C | — | — | — | — |
| V191992 | D | — | D | B | — | — | — | — |
| V191971 | D | — | C | B | — | — | — | — |
| V191956 | D | — | D | D | — | — | — | — |
| V191939 | B | — | A | A | — | — | — | — |
| V190526 | D | — | B | A | — | — | — | — |
| V190446 | A | — | A | A | — | — | — | — |
| V190441 | D | — | D | D | — | — | — | — |
| V192039 | D | — | — | D | — | — | — | — |
| V192038 | D | — | — | D | — | — | — | — |
| V192037 | — | — | — | A | — | — | — | — |
| V192036 | B | — | — | A | — | — | — | — |
| V191989 | A | — | — | A | — | — | — | — |
| V191988 | A | — | — | A | — | — | — | — |
| V191970 | D | — | — | — | — | — | — | — |
| V191968 | — | — | — | D | — | — | — | — |
| V191963 | D | — | C | C | — | — | — | — |
| V191945 | B | — | A | A | — | — | — | — |
| V191943 | A | — | A | A | — | — | — | — |
| V191920 | A | — | A | A | — | — | — | — |
| V191962 | D | — | D | D | — | — | — | — |
| V191955 | A | — | A | A | — | — | — | — |
| V191954 | A | — | A | A | — | — | — | — |
| V191948 | D | — | D | D | — | — | — | — |
| V191932 | B | — | A | A | — | — | — | — |
| V191930 | C | — | B | A | — | — | — | — |
| V191906 | B | — | A | A | — | — | — | — |
| V191902 | D | — | D | B | — | — | — | — |
| V190559 | A | — | A | A | — | — | — | — |
| V190547 | C | — | A | A | — | — | — | — |
| V190546 | A | — | A | A | — | — | — | — |
| V190515 | A | — | A | A | — | — | — | — |
| V191915 | A | — | A | A | — | — | — | — |
| V191911 | B | — | A | A | — | — | — | — |
| V191904 | A | — | A | A | — | — | — | — |
| V191885 | B | — | A | A | — | — | — | — |
| V190517 | A | — | A | A | — | — | — | — |
| V190516 | B | — | A | A | — | — | — | — |
| V190514 | A | — | A | A | — | — | — | — |
| V190511 | A | — | A | A | — | — | — | — |
| V188356 | A | — | A | A | — | — | — | — |
| V191886 | A | — | A | A | — | — | — | — |
| V190545 | A | — | A | A | A | A | A | — |
| V190538 | D | — | D | D | — | — | — | — |
| V190520 | B | — | A | A | — | — | — | — |
| V190512 | A | — | A | A | — | — | — | — |
| V190505 | A | — | A | A | — | — | — | — |
| V190449 | A | — | A | A | — | — | — | — |
| V190410 | D | — | B | A | — | — | — | — |
| V188354 | A | — | A | A | — | — | — | — |
| V190522 | A | — | A | A | — | — | — | — |
| V190521 | D | — | C | B | — | — | — | — |
| V190518 | C | — | B | A | — | — | — | — |
| V190509 | D | — | D | C | — | — | — | — |
| V190506 | B | — | A | A | — | — | — | — |
| V190459 | B | — | A | A | — | — | — | — |
| V190414 | D | — | D | C | — | — | — | — |
| V188406 | A | — | A | A | — | — | — | — |
| V188405 | A | — | A | A | — | — | — | — |
| V188398 | A | — | A | A | — | — | — | — |
| V188330 | A | — | A | A | — | — | — | — |
| V188329 | A | — | A | A | — | — | — | — |
| V188366 | B | — | A | A | — | — | — | — |
| V188347 | A | — | A | A | A | A | A | A |
| V190415 | D | — | D | D | — | — | — | — |
| V188382 | A | — | A | A | — | — | — | — |
| V188376 | B | — | A | A | — | — | — | — |
| V188365 | B | — | A | A | — | — | — | — |
| V188360 | A | — | A | A | — | — | — | — |
| V188355 | A | — | A | A | — | — | — | — |
| V188339 | A | — | A | A | — | — | — | — |
| V188319 | D | — | B | A | — | — | — | — |
| V190413 | C | — | B | A | — | — | — | — |
| V190411 | B | — | B | A | — | — | — | — |
| V190408 | A | — | A | A | — | — | — | — |
| V188392 | A | — | A | A | — | — | — | — |
| V188384 | A | — | A | A | — | — | — | — |
| V188381 | B | — | A | A | — | — | — | — |
| V188373 | A | — | A | A | — | — | — | — |
| V188361 | A | — | A | A | — | — | — | — |
| V188345 | A | — | A | A | — | — | — | — |
| V188344 | A | — | A | A | — | — | — | — |
| V188340 | B | — | A | A | — | — | — | — |
| V188324 | B | — | B | A | — | — | — | — |
| V188323 | C | — | C | A | — | — | — | — |
| V188316 | A | — | A | A | — | — | — | — |
| V188314 | A | — | A | A | — | — | — | — |
| V188303 | A | — | A | A | — | — | — | — |
| V188297 | A | — | A | A | — | — | — | — |
| V188383 | A | — | A | A | — | — | — | — |
| V188380 | A | — | A | A | — | — | — | — |
| V188379 | A | — | A | A | — | — | — | — |
| V188378 | A | — | A | A | — | — | — | — |
| V188377 | A | — | A | A | — | — | — | — |
| V188348 | A | — | A | A | — | — | — | — |
| V188342 | C | — | B | A | — | — | — | — |
| V188341 | A | — | A | A | — | — | — | — |
| V188311 | A | — | A | A | — | — | — | — |
| V188307 | A | — | A | A | — | — | — | — |
| V188306 | A | — | A | A | — | — | — | — |
| V188299 | B | — | A | A | — | — | — | — |
| V188337 | A | — | A | A | — | — | — | — |
| V188331 | A | — | A | A | — | — | — | — |
| V188327 | A | — | A | A | — | — | — | — |
| V188322 | A | — | A | A | — | — | — | — |
| V188321 | D | — | B | B | — | — | — | — |
| V188318 | A | — | A | A | — | — | — | — |
| V188315 | C | — | A | A | — | — | — | — |
| V188310 | A | — | A | A | — | — | — | — |
| V188298 | B | — | A | A | — | — | — | — |
| V188296 | A | — | A | A | — | — | — | — |
| V188305 | A | A | — | A | — | — | — | — |
| V188304 | A | A | — | A | — | — | — | — |
| V188291 | A | A | — | A | — | — | — | — |
| V188289 | A | A | A | A | — | — | — | — |
| V188288 | C | A | — | A | — | — | — | — |
| V188287 | A | A | A | A | — | — | — | — |
| V188286 | D | D | — | D | — | — | — | — |
| V188276 | A | A | A | A | — | — | — | — |
| V188275 | B | A | — | A | — | — | — | — |
| V188274 | A | A | A | A | — | — | — | — |
| V188273 | D | C | — | B | — | — | — | — |
| V188272 | C | A | — | A | — | — | — | — |
| V188271 | B | A | — | A | — | — | — | — |
| V188270 | B | A | — | A | — | — | — | — |
| V188269 | D | B | — | A | — | — | — | — |
| V188228 | D | D | — | D | — | — | — | — |
| V188227 | C | C | — | A | — | — | — | — |
| V188203 | B | A | — | A | — | — | — | — |
| V188149 | D | B | — | A | — | — | — | — |
| V188080 | A | A | — | A | — | — | — | — |
| V187808 | A | A | — | A | — | — | — | — |
| V188229 | B | B | A | A | — | — | — | — |
| V188226 | A | A | — | A | — | — | — | — |
| V188225 | A | A | A | A | — | — | — | — |
| V188150 | A | A | — | A | — | — | — | — |
| V188144 | A | A | — | A | — | — | — | — |
| V188131 | B | A | — | A | — | — | — | — |
| V188084 | A | A | — | A | — | — | — | — |
| V188076 | C | B | — | A | — | — | — | — |
| V188071 | B | A | — | A | — | — | — | — |
| V188035 | A | A | — | A | — | — | — | — |
| V188210 | B | A | — | A | — | — | — | — |
| V188206 | A | A | — | A | — | — | — | — |
| V188205 | C | B | — | A | — | — | — | — |
| V188202 | A | A | — | A | — | — | — | — |
| V188171 | B | B | A | A | — | — | — | — |
| V188170 | A | A | — | A | — | — | — | — |
| V188165 | B | — | C | A | — | — | — | — |
| V188162 | D | — | — | A | — | — | — | — |
| V188152 | D | B | — | A | — | — | — | — |
| V188143 | A | A | A | A | — | — | — | — |
| V188108 | D | D | — | C | — | — | — | — |
| V188089 | B | A | — | A | — | — | — | — |
| V188086 | B | A | B | A | — | — | — | — |
| V188085 | C | A | — | A | — | — | — | — |
| V188079 | D | D | — | B | — | — | — | — |
| V187925 | A | A | — | A | — | — | — | — |
| V188188 | D | A | — | A | — | — | — | — |
| V188175 | D | B | — | A | — | — | — | — |
| V188172 | D | D | — | D | — | — | — | — |
| V188167 | D | — | — | D | — | — | — | — |
| V188164 | D | C | — | B | — | — | — | — |
| V188161 | D | B | — | A | — | — | — | — |
| V188160 | C | B | — | A | — | — | — | — |
| V188154 | A | A | — | A | — | — | — | — |
| V188151 | A | A | A | A | — | — | — | — |
| V188148 | A | A | — | A | — | — | — | — |
| V188147 | B | A | — | A | — | — | — | — |
| V188138 | A | A | A | A | — | — | — | — |
| V188135 | C | C | — | B | — | — | — | — |
| V188088 | D | B | — | A | — | — | — | — |
| V188050 | A | A | — | A | — | — | — | — |
| V187944 | C | B | — | A | — | — | — | — |
| V188018 | D | — | D | D | — | — | — | — |
| V188019 | D | — | D | D | — | — | — | — |

*A = 0 to <10; B = 10 to <20; C = 20 to <30; D = ≥30; Compounds not tested in any of the IVTT assays were excluded from Table E1C

### Example 5: In Vitro Transcription Translation Data and Cell Viability Data

### Cell Viability Data

Count and dispense 2,000-10,000 cells per well (100 μL volume) in black 96-well tissue culture microplate and let cells adhere overnight. Add compound dissolved in 100% DMSO to each well as needed and incubate cells in standard culture conditions for 24-72 hrs. Final DMSO concentration in each well should be 0.1-0.5%. At the desired time point, gently aspirate compound containing media and replace with PBS to wash. Repeat wash step for 2 total washes. Remove PBS and add 100 μL CellTiter-Fluor reagent (Promega) reagent to each well. Seal plate and incubate 1 hr at room temperature with orbital shaking. Analyze fluorescence using plate reader to quantify viable cells compared with vehicle (DMSO) control.

TABLE E2
SU Cell Viability Data (HT) *

| | HEK | | | | | NCI- | | | | | |
|---|---|---|---|---|---|---|---|---|---|---|---|
| | 293T | HPAF- | KLE | LS411N | MCF7 | H1915 | HCC38 | HEPG2 | KATO- | MS751 | T47D |
| | IC₅₀ | II IC₅₀ | IC₅₀ | IC₅₀ | IC₅₀ | IC₅₀ | IC₅₀ | IC₅₀ | III IC₅₀ | IC₅₀ | IC₅₀ |
| Cmpd | (μM) | (μM) | (μM) | (μM) | (μM) | (μM) | (μM) | (μM) | (μM) | (μM) | (μM) |
| V187189 | A | B | B | A | B | B | B | — | B | B | B |
| V187190 | A | B | A | A | A | A | B | A | A | A | A |
| V187509 | A | B | B | B | B | B | B | C | B | A | B |
| V187536 | B | C | C | C | C | C | C | C | C | B | C |
| V187685 | D | — | — | — | C | — | — | — | — | — | — |
| V187686 | D | D | — | D | C | D | — | — | — | — | — |
| V187819 | D | — | — | — | C | — | — | — | — | — | — |
| V187594 | D | — | — | — | C | — | — | — | — | — | — |
| V187783 | B | — | — | — | C | — | — | — | — | — | — |
| V187822 | D | — | — | — | D | — | — | — | — | — | — |
| V187823 | D | — | — | — | — | — | — | — | — | — | — |
| V187825 | D | — | — | — | D | — | — | — | — | — | — |
| V187826 | C | — | — | — | C | — | — | — | — | — | — |
| V187827 | D | — | — | — | — | — | — | — | — | — | — |
| V187878 | D | D | D | C | B | D | — | C | — | — | — |
| V187879 | D | — | — | — | C | — | — | — | — | — | — |
| V187917 | A | C | B | B | B | B | — | C | — | — | — |
| V187940 | B | — | — | — | C | — | — | — | — | — | — |
| V187987 | A | B | B | A | A | A | B | C | B | B | A |
| V187988 | B | C | B | B | B | B | — | D | — | — | — |
| V188017 | C | — | — | — | C | — | — | — | — | — | — |
| V188053 | C | C | C | C | C | C | — | — | — | — | — |

* A = 0 to <0.25; B = 0.25 to <1; C = 1 to <4; D = ≥4; Compounds not tested in any of the cell viability assays were excluded from Table E2

### INCORPORATION BY REFERENCE

The present application refers to various issued patent, published patent applications, scientific journal articles, and other publications, all of which are incorporated herein by reference. The details of one or more embodiments of the present disclosure are set forth herein. Other features, objects, and advantages of the present disclosure will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A compound of Formula (I): 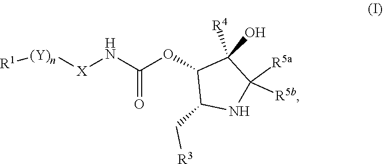
or a pharmaceutically acceptable salt thereof, wherein:
X is —CH(R²)— or —NH—;
R¹ is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{1a}, or —N(R^{1a})₂;
each instance of R^{1a} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, or an oxygen protecting group when attached to an oxygen atom, or two instances of R^{1a} are joined together with their intervening atom to form an optionally substituted heterocyclic ring or optionally substituted heteroaryl ring;
R² is hydrogen or optionally substituted alkyl;
or R¹ and R² are joined together with their intervening atoms to form optionally substituted carbocyclyl, or optionally substituted heterocyclyl;
R³ is optionally substituted carbocyclyl, optionally substituted heterocyclyl, optionally substituted aryl, or optionally substituted heteroaryl;
R⁴ is hydrogen or optionally substituted alkyl;
R^{5a} and R^{5b} are each hydrogen; or R^{5a} and R^{5b} are joined together with their intervening atom to form optionally substituted carbocyclyl;
each instance of Y is independently —C(R^{Y})₂—, —O—, or —N(R^{1a})—;
each instance of R is independently hydrogen or halogen, or two instances of R are taken together to form ═O; and
n is 0, 1, 2, 3, 4, or 5.

2. The compound of claim 1, wherein the compound is of Formula (II): 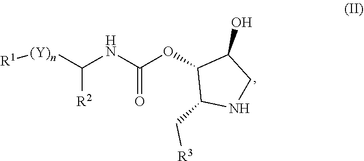
or a pharmaceutically acceptable salt thereof.

3. The compound of claim 1, or a pharmaceutically acceptable salt thereof, wherein the compound is of Formula (IV-a): 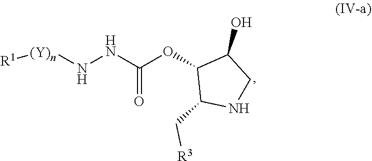
or a pharmaceutically acceptable salt thereof.

4. The compound of any one of claims 1-3, or a pharmaceutically acceptable salt thereof, wherein R¹ is hydrogen, optionally substituted alkyl, optionally substituted alkenyl, or optionally substituted alkynyl.

5. The compound of any one of claims 1-4, or a pharmaceutically acceptable salt thereof, wherein R¹ is hydrogen, methyl, ethyl, isopropyl,

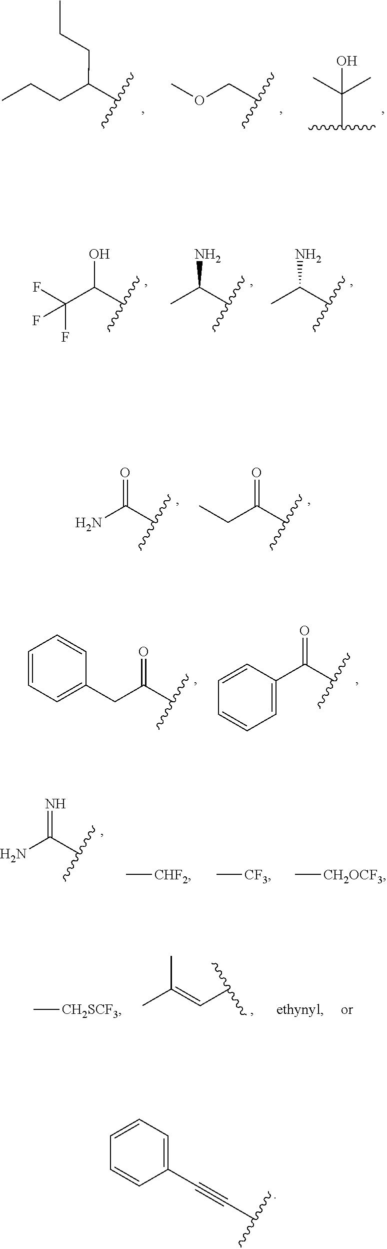

6. The compound of any one of claims 1-3, or a pharmaceutically acceptable salt thereof, wherein R¹ is optionally substituted carbocyclyl.

7. The compound of any one of claims 1-3 or 6, or a pharmaceutically acceptable salt thereof, wherein R¹ is optionally substituted cyclopropyl, optionally substituted cyclobutyl, optionally substituted cyclobutenyl, optionally substituted cyclopentyl, or optionally substituted cyclohexyl.

8. The compound of any one of claims 1-3, 6, or 7, or a pharmaceutically acceptable salt thereof, wherein R¹ is

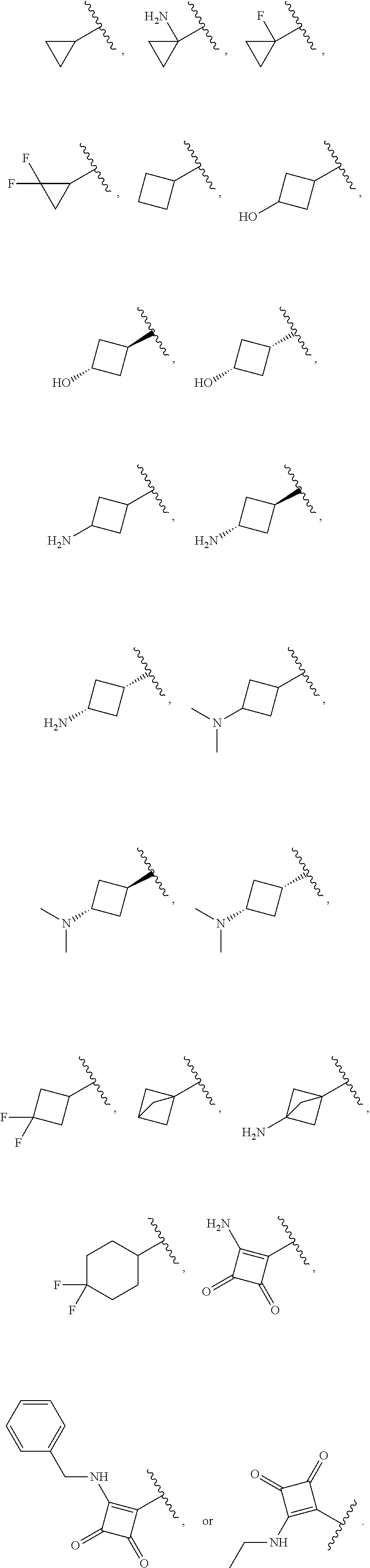

9. The compound of any one of claims 1-3, or a pharmaceutically acceptable salt thereof, wherein R¹ is optionally substituted heterocyclyl.

10. The compound of any one of claims 1-3 or 9, or a pharmaceutically acceptable salt thereof, wherein R¹ is optionally substituted tetrahydrofuranyl, optionally substituted tetrahydropyranyl, optionally substituted azetidinyl, optionally substituted pyrrolidinyl, optionally substituted imidazolidinyl, optionally substituted piperidinyl, optionally substituted piperazinyl, optionally substituted tetrahydroimidazopyrazinyl, optionally substituted tetrahydrothiopyranyl, optionally substituted 2,6-diazaspiro[3.3]heptyl, optionally substituted 6-azaspiro[3.4]octyl, 2,6-diazaspiro[3.4]octyl, optionally substituted 2,5,8-triazaspiro[3.5]nonyl, or optionally substituted 1,4,9-triazaspiro[5.5]undecyl.

11. The compound of any one of claims 1-3, 9, or 10, or a pharmaceutically acceptable salt thereof, wherein optionally substituted tetrahydrofuranyl, optionally substituted azetidinyl, optionally substituted pyrrolidinyl, optionally substituted imidazolidinyl, optionally substituted piperidinyl, optionally substituted piperazinyl, optionally substituted tetrahydrothiopyranyl, optionally substituted 2,6-diazaspiro[3.3]heptyl, optionally substituted 6-azaspiro[3.4]octyl, optionally substituted 2,6-diazaspiro[3.4]octyl, or optionally substituted 1,4,9-triazaspiro[5.5]undecyl.

12. The compound of any one of claims 1-3 or 9-11, or a pharmaceutically acceptable salt thereof, wherein R¹ is

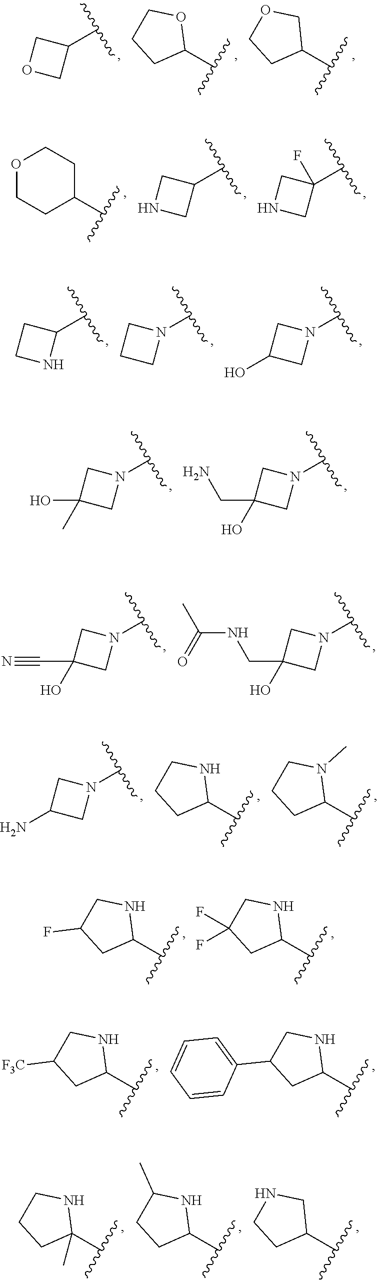

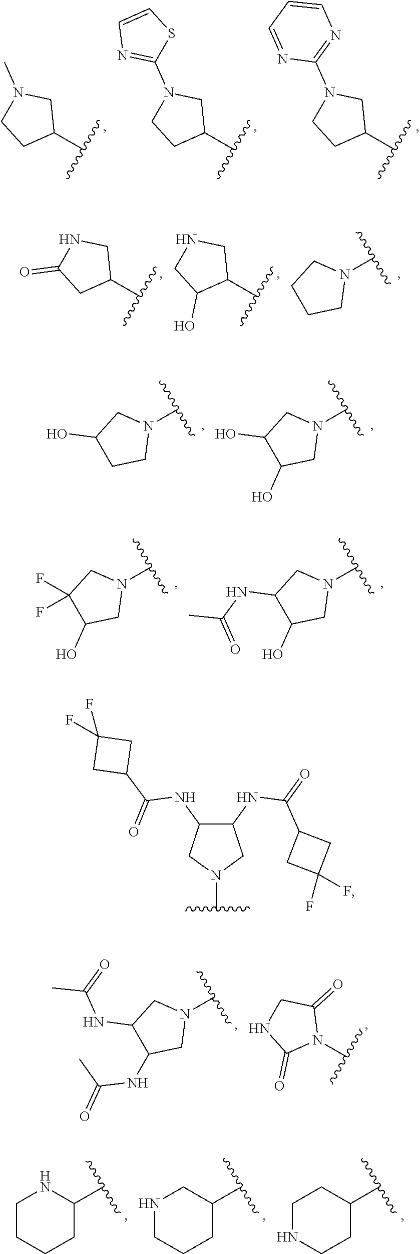

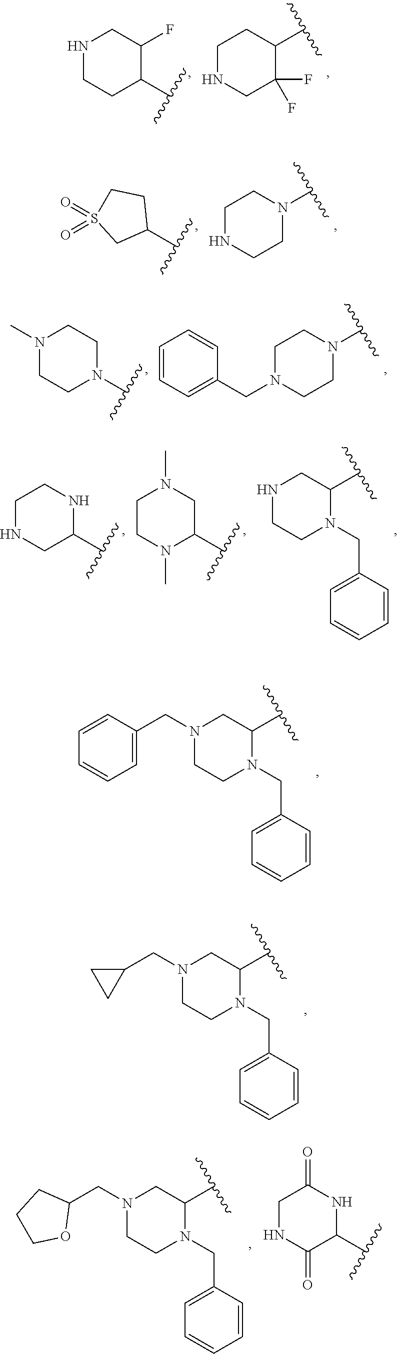

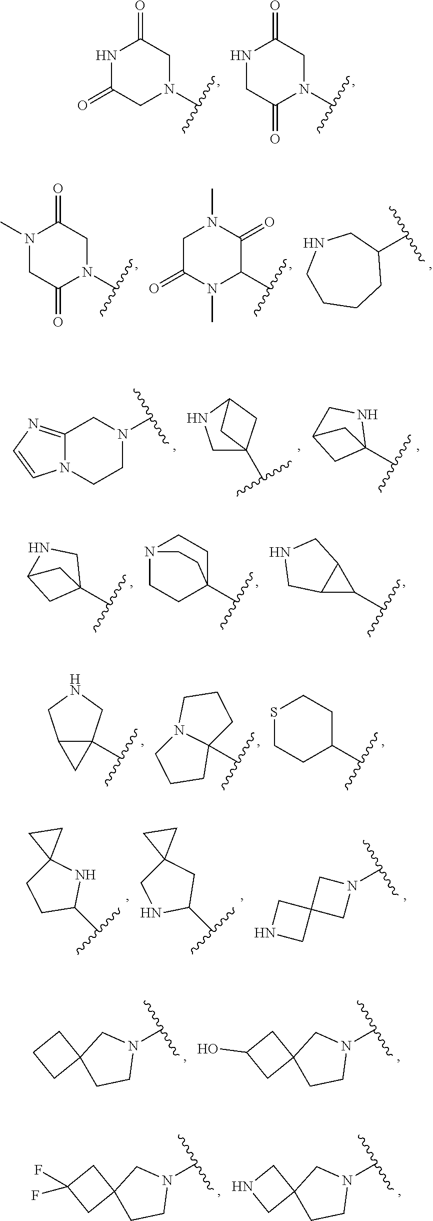

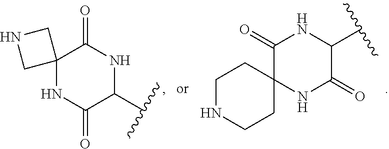 [SMILES: *C1NC(=O)C2(CNC2)NC1=O ; *C1NC(=O)C2(CCNCC2)NC1=O]

13. The compound of any one of claims 1-3 or 9-12, or a pharmaceutically acceptable salt thereof, wherein R¹ is

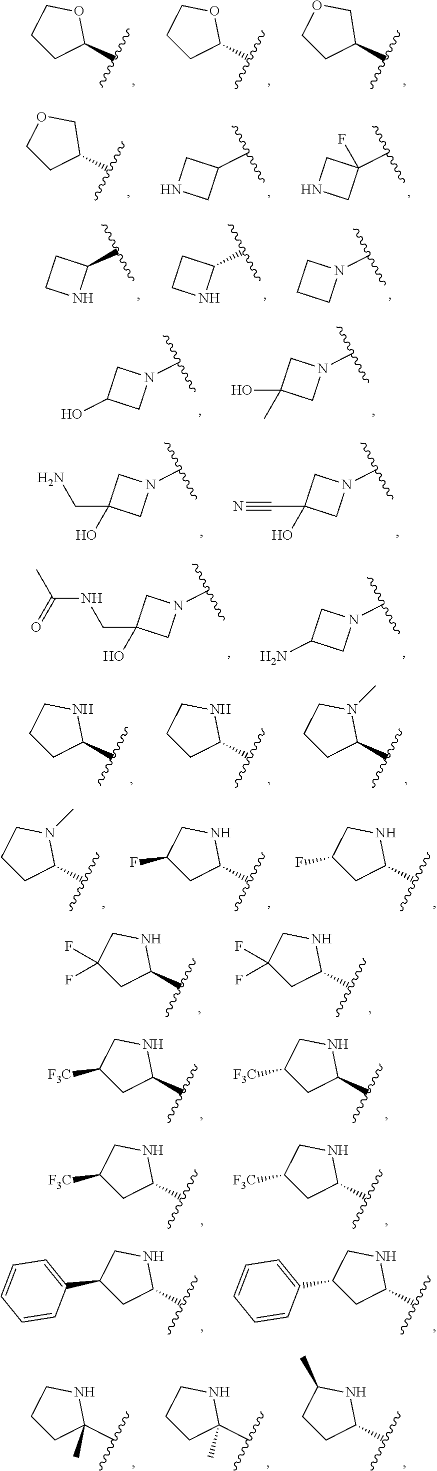

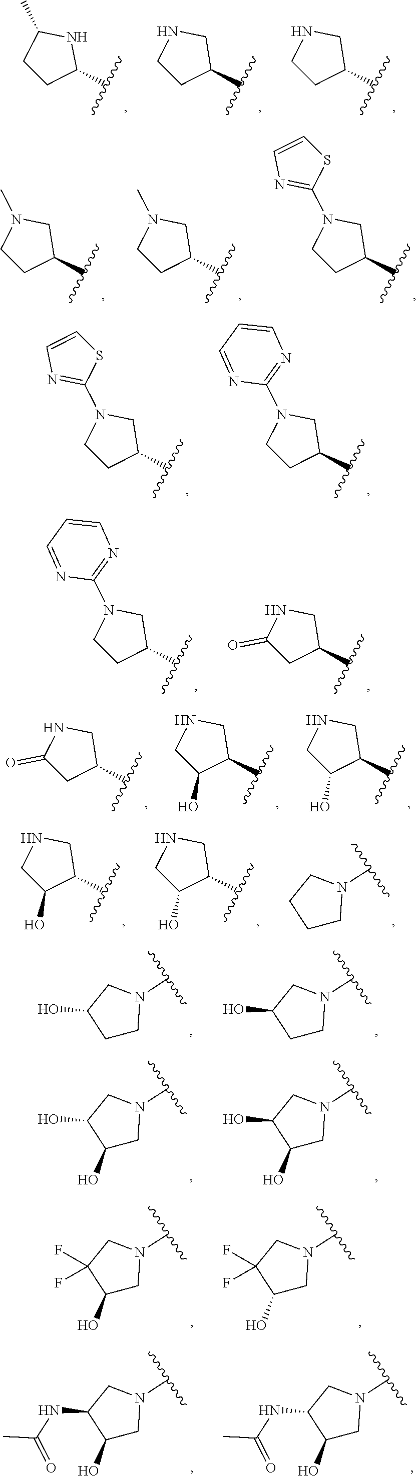

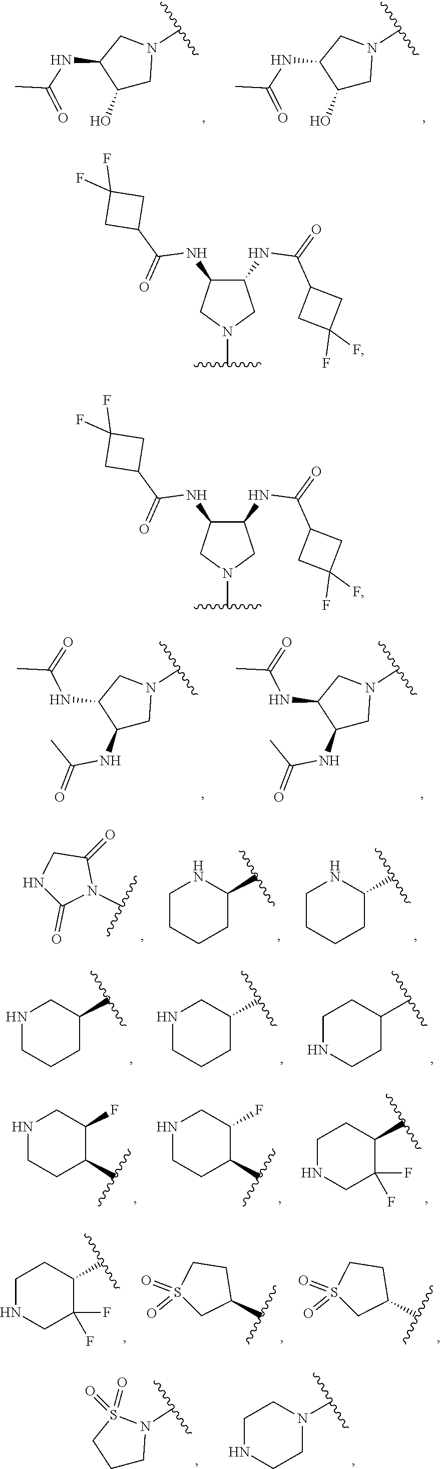

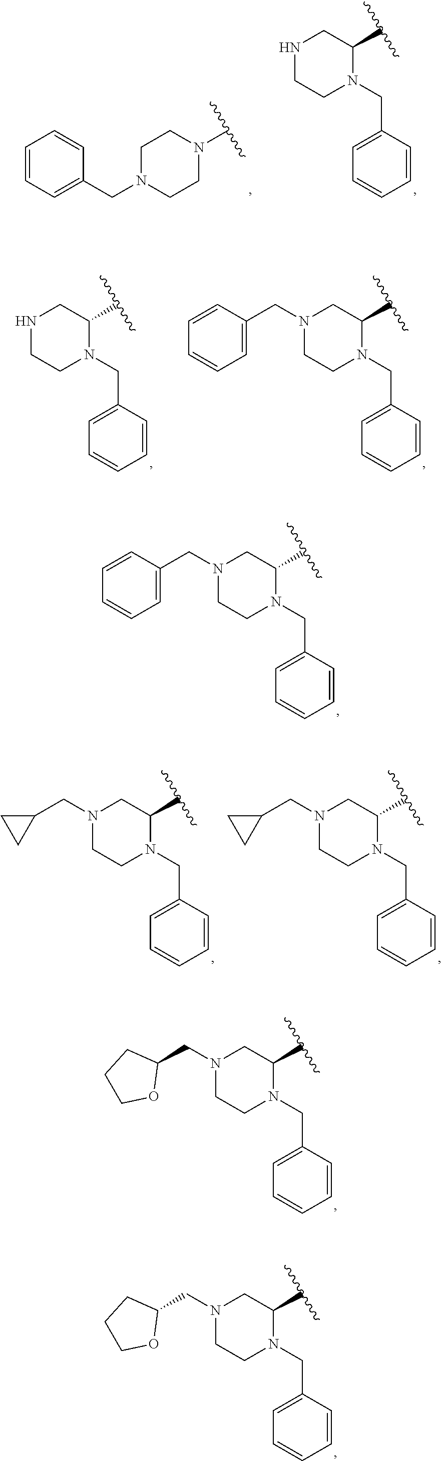

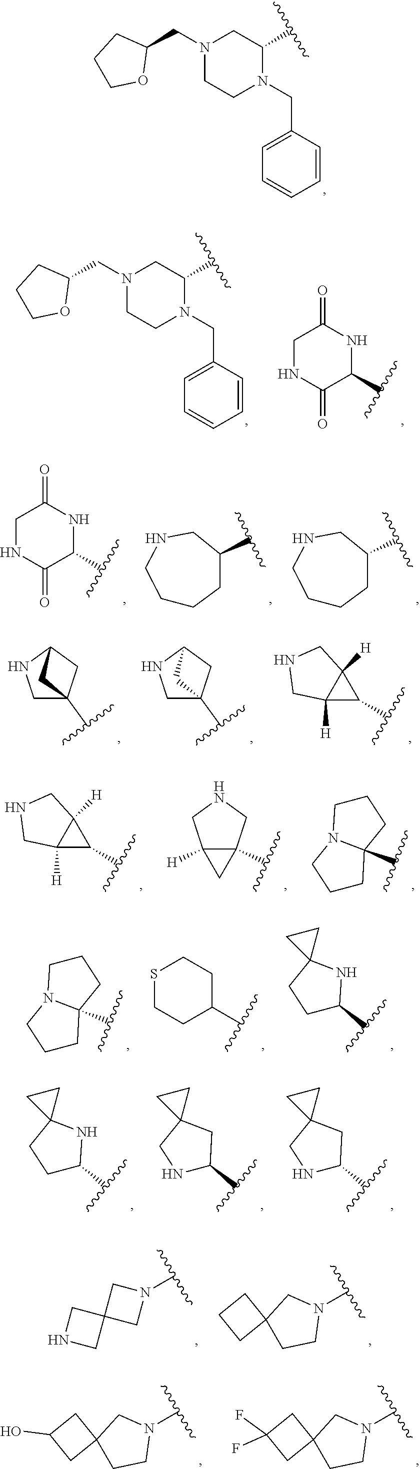

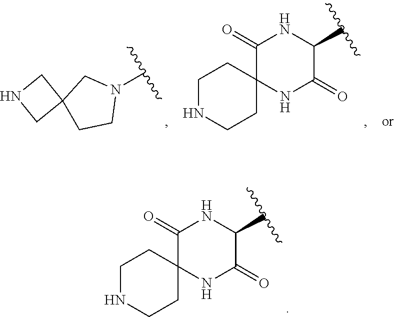 [SMILES: *N1CCC2(CNC2)C1 ; *[C@@H]1NC(=O)C2(CCNCC2)NC1=O ; *[C@@H]1NC(=O)C2(CCNCC2)NC1=O]

14. The compound of any one of claims 1-3, or a pharmaceutically acceptable salt thereof, wherein R¹ is optionally substituted aryl.

15. The compound of any one of claims 1-3 or 14, or a pharmaceutically acceptable salt thereof, wherein R¹ is phenyl optionally substituted with halogen, optionally substituted alkyl, optionally substituted aryl, —OR^{1a}, —N(R^{1a})₂, —C(═NR^{A})N(R^{A})₂, and/or —B(OR^{A})₂.

16. The compound of any one of claims 1-3, 14, or 15, or a pharmaceutically acceptable salt thereof, wherein R¹ is

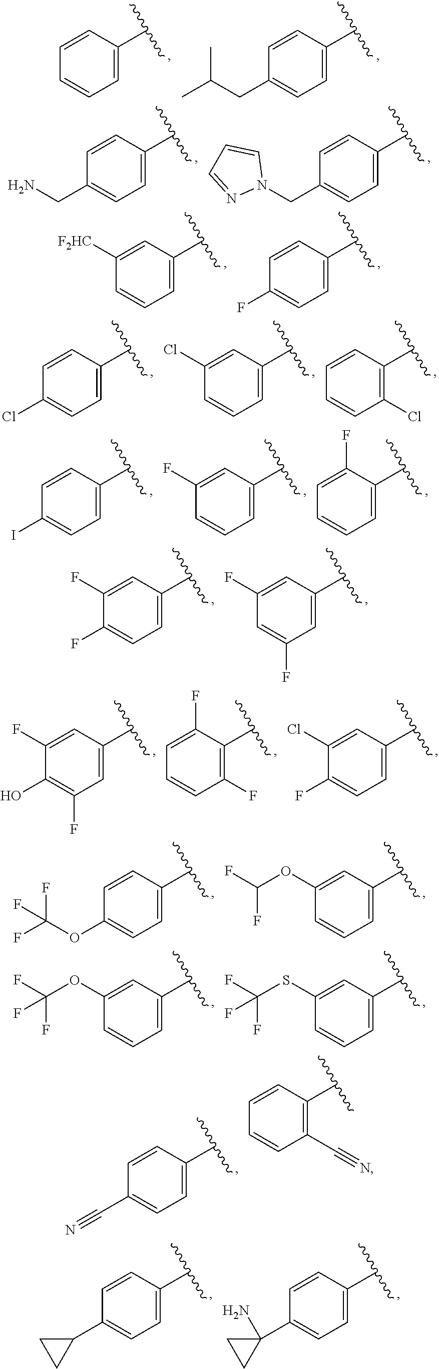

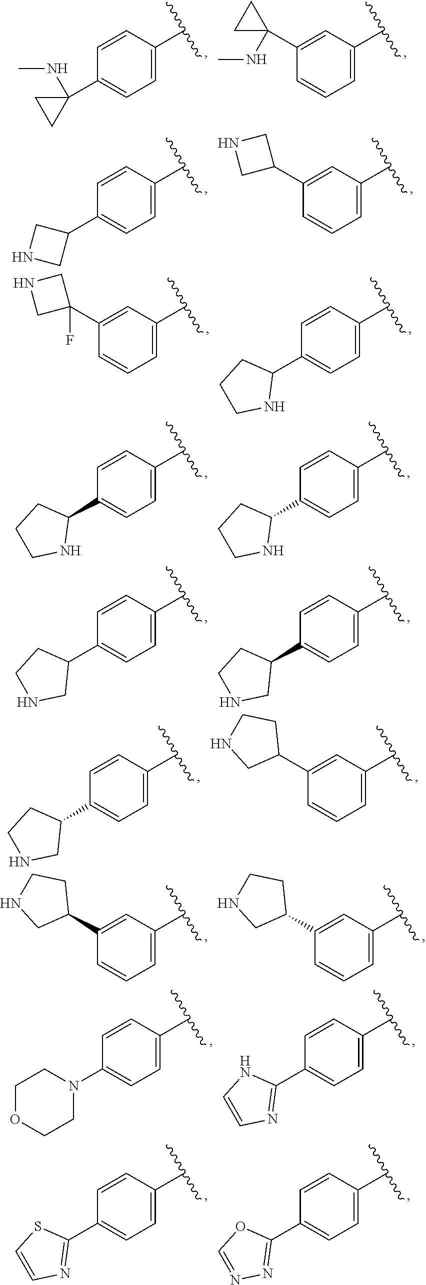

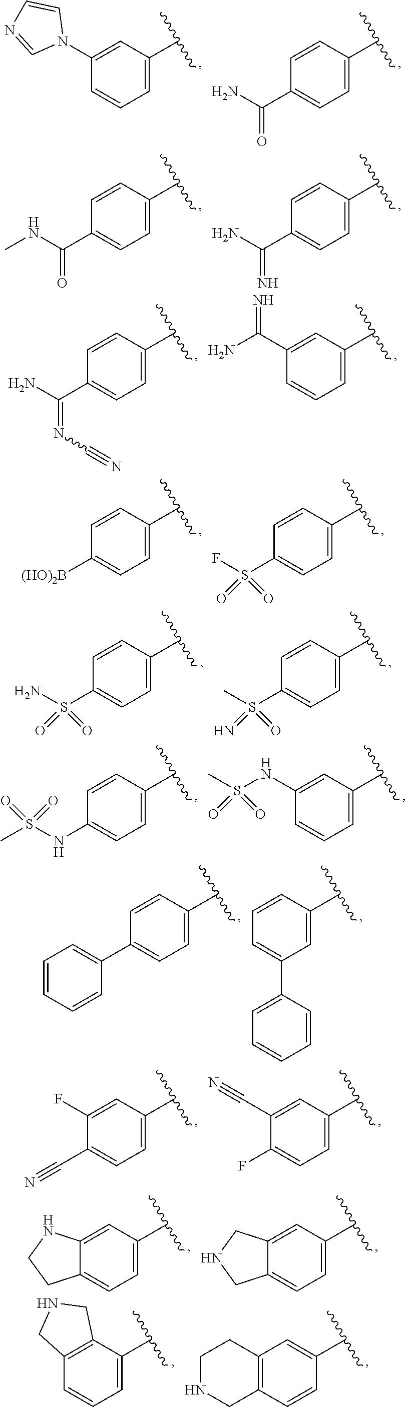

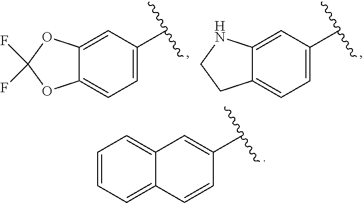

17. The compound of any one of claims 1-3, or a pharmaceutically acceptable salt thereof, wherein R¹ is optionally substituted heteroaryl.

18. The compound of any one of claims 1-3 or 17, or a pharmaceutically acceptable salt thereof, wherein R¹ is optionally substituted pyridyl, optionally substituted pyrazinyl, optionally substituted pyrimidinyl, optionally substituted pyridazinyl, optionally substituted pyrazolyl, optionally substituted imidazolyl, optionally substituted triazolyl, optionally substituted tetrazolyl, optionally substituted benzimidazolyl, optionally substituted benzotriazolyl, optionally substituted purinyl, optionally substituted thiophenyl, optionally substituted benzothiophenyl, optionally substituted oxazolyl, optionally substituted isoxazolyl, optionally substituted oxadiazolyl, optionally substituted benzooxadiazolyl, optionally substituted thiazolyl, optionally substituted thiadiazolyl, or optionally substituted benzothiadiazolyl.

19. The compound of any one of claims 1-3, 17, or 18, or a pharmaceutically acceptable salt thereof, wherein R¹ is optionally substituted pyridyl, optionally substituted pyrimidinyl, optionally substituted pyridazinyl, optionally substituted pyrazolyl, optionally substituted imidazolyl, optionally substituted triazolyl, optionally substituted tetrazolyl, optionally substituted benzimidazolyl, optionally substituted purinyl, optionally substituted oxazolyl, optionally substituted isoxazolyl, or optionally substituted thiazolyl.

20. The compound of any one of claims 1-3 or 17-19, or a pharmaceutically acceptable salt thereof, wherein R¹ is

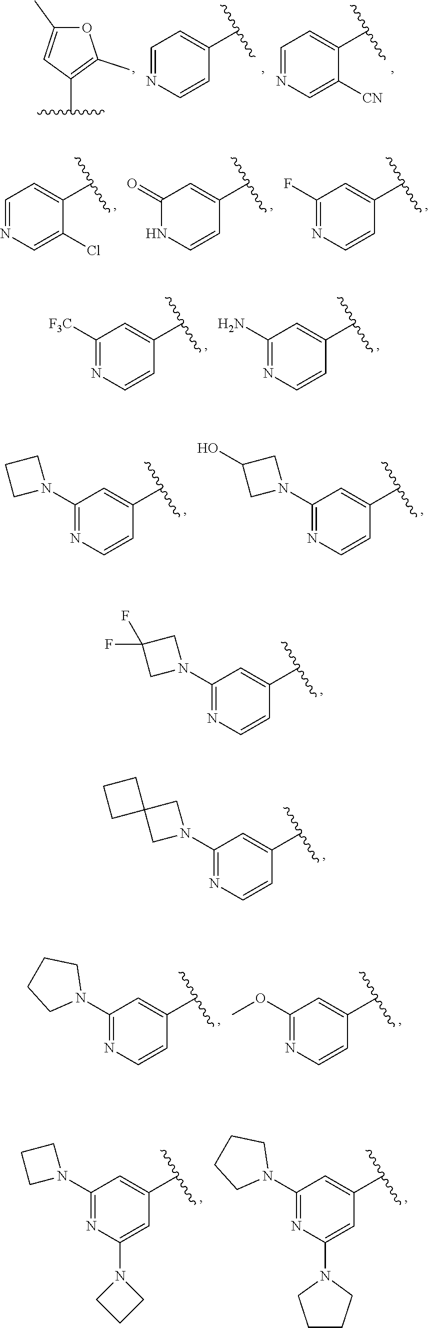

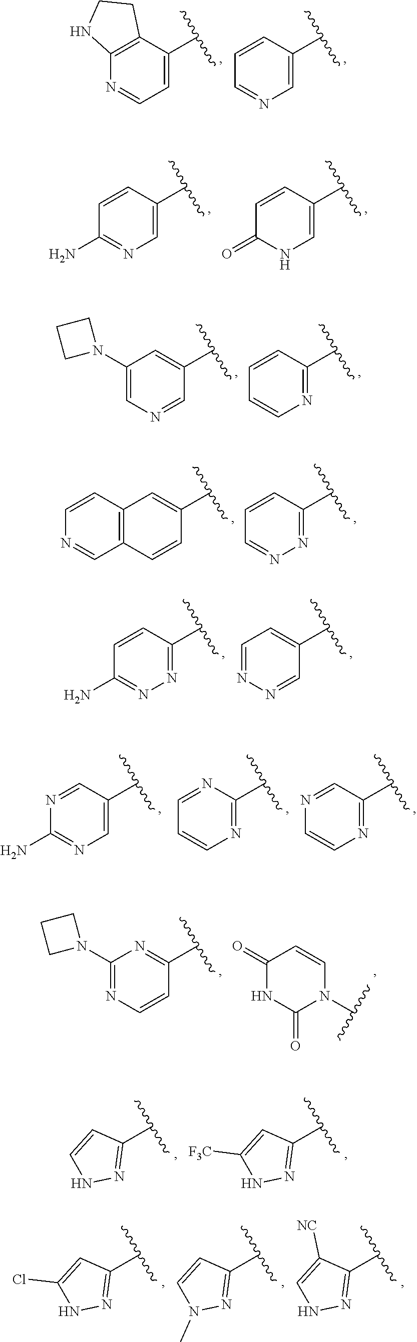

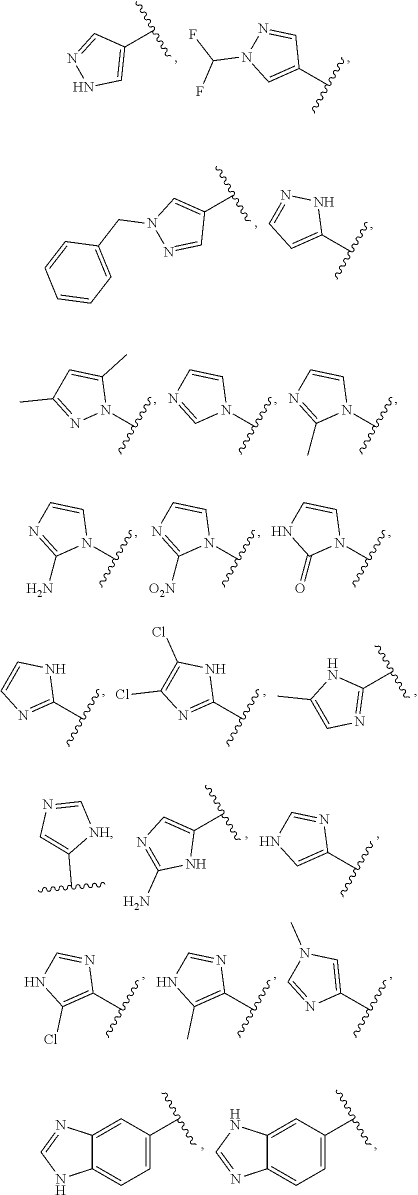

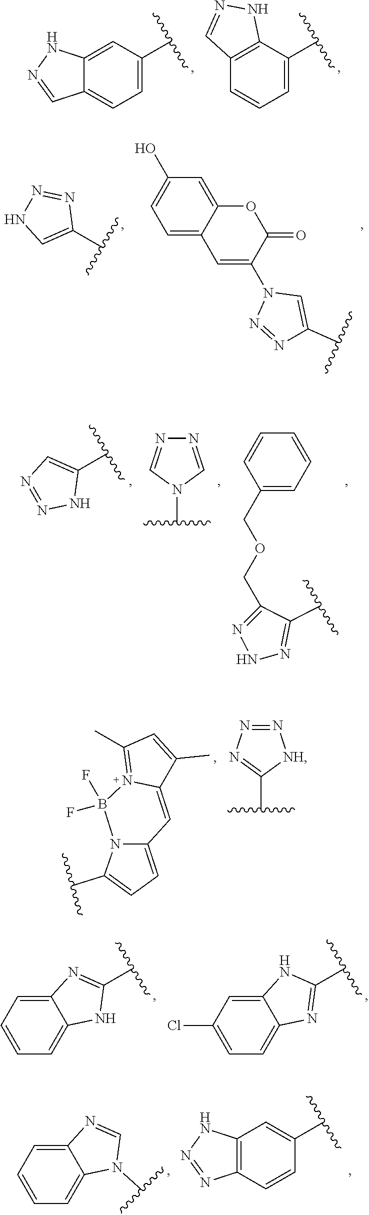

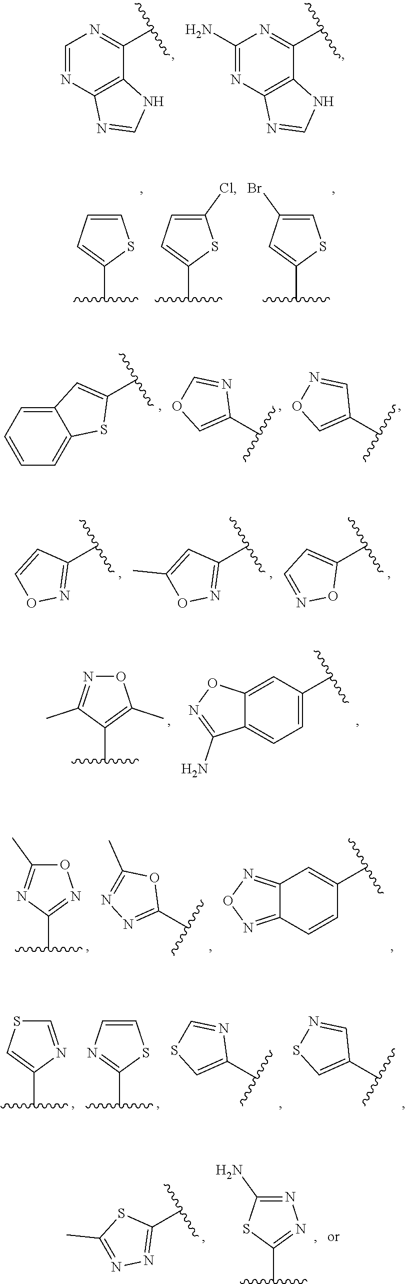

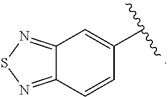 [SMILES: *c1ccc2nsnc2c1]

21. The compound of any one of claims 1-3, or a pharmaceutically acceptable salt thereof, wherein R¹ is —OR^{1a} or —N(R^{1a})₂.

22. The compound of any one of claims 1-3 or 21, or a pharmaceutically acceptable salt thereof, wherein R¹ is —OH, —OCHF₂—OCF₃,

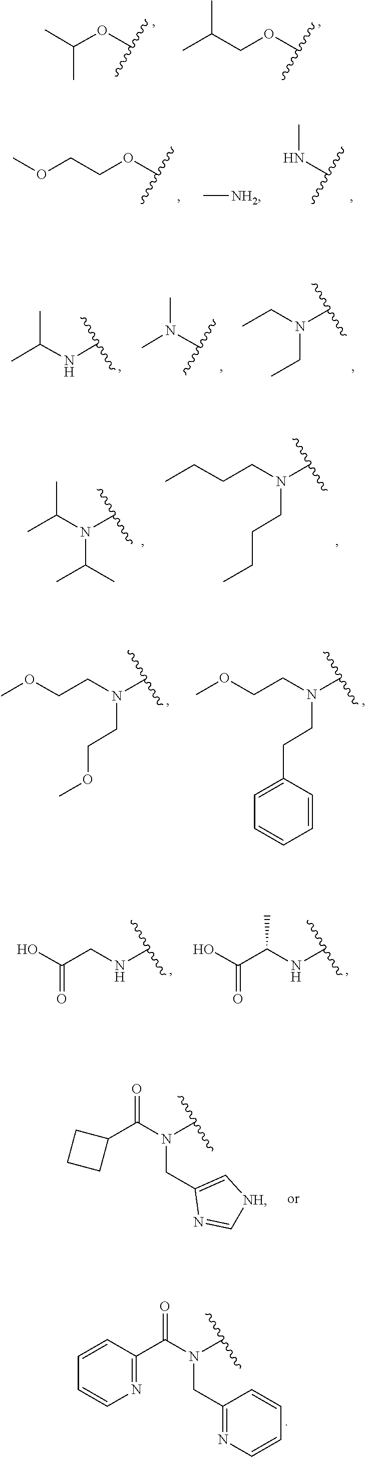

23. The compound of any one of claim 1, 2, or 4-22, or a pharmaceutically acceptable salt thereof, wherein R² is hydrogen.

24. The compound of any one of claims 1-23, or a pharmaceutically acceptable salt thereof, wherein n is 0.

25. The compound of any one of claims 1-23, or a pharmaceutically acceptable salt thereof, wherein n is 1.

26. The compound of any one of claims 1, 2, or 4-25, wherein the compound is of Formula (II-a-1) or (II-a-2): 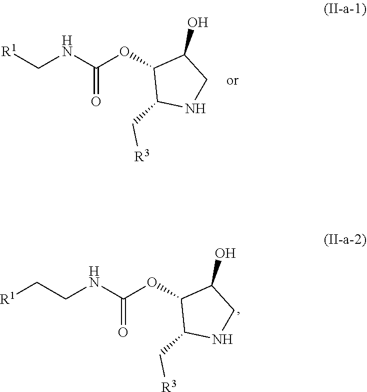
or a pharmaceutically acceptable salt thereof.

27. The compound of any one of claims 1-26, or a pharmaceutically acceptable salt thereof, wherein at least one instance of Y is C(R^{Y})₂.

28. The compound of any one of claims 1-27, or a pharmaceutically acceptable salt thereof, wherein at least one instance of Y is CH₂.

29. The compound of any one of claims 1-25, or a pharmaceutically acceptable salt thereof, wherein at least one instance of Y is —O— or —N(R^{1a})—.

30. The compound of any one of claims 1-3, 24, 25, 27, or 28, or a pharmaceutically acceptable salt thereof, wherein R¹ and R² are joined together with their intervening atoms to form optionally substituted carbocyclyl.

31. The compound of any one of claims 1-3, 24, 25, 27, 28, or 30, or a pharmaceutically acceptable salt thereof, wherein R¹ and R² are joined together with their intervening atoms to form optionally substituted cyclobutyl, optionally substituted cyclohexyl, optionally substituted spiro[2.2]pentyl, optionally substituted spiro[2.3]hexyl, or optionally substituted spiro[3.3]heptyl.

32. The compound of any one of claims 1-3, 24, 25, 27, 28, 30, or 31, or a pharmaceutically acceptable salt thereof, wherein R¹ and R² are joined together with their intervening atoms to form optionally substituted cyclobutyl.

33. The compound of any one of claims 1-3, 24, 25, 27, 28, or 30-32, or a pharmaceutically acceptable salt thereof, wherein R¹ and R² are joined together with their intervening atoms to form

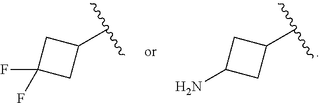 [SMILES: *C1CC(F)(F)C1 ; *C1CC(N)C1]

34. The compound of any one of claims 1-3, 24, 25, or 27-29, or a pharmaceutically acceptable salt thereof, wherein R¹ and R² are joined together with their intervening atoms to form optionally substituted heterocyclyl.

35. The compound of any one of claims 1-3, 24, 25, 27-29, or 34, or a pharmaceutically acceptable salt thereof, wherein R¹ and R² are joined together with their intervening atoms to form optionally substituted oxetanyl, optionally substituted tetrahydrofuranyl, optionally substituted tetrahydropyranyl, optionally substituted azetidinyl, optionally substituted pyrrolidinyl, optionally substituted 2-azaspiro[3.3]heptyl, optionally substituted 2,6-diazaspiro[3.4]octanyl, optionally substituted tetrahydrothiophenyl, optionally substituted dithiolanyl, or optionally substituted 1-imino-1-oxo-hexahydro-1λ⁶-thiopyranyl.

36. The compound of any one of claims 1-3, 24, 25, 27-29, 34, or 35, or a pharmaceutically acceptable salt thereof, wherein R¹ and R² are joined together with their intervening atoms to form optionally substituted azetidinyl or optionally substituted pyrrolidinyl.

37. The compound of any one of claims 1-3, 24, 25, 27-29, or 34-36, or a pharmaceutically acceptable salt thereof, wherein R¹ and R² are joined together with their intervening atoms to form

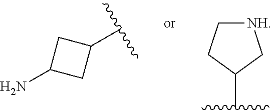 [SMILES: *C1CC(N)C1 ; *C1CCNC1]

38. A compound of Formula (V): 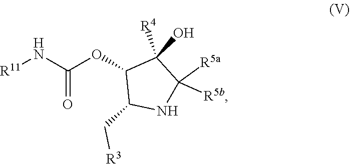
or a pharmaceutically acceptable salt thereof, wherein:
R³ is optionally substituted carbocyclyl, optionally substituted heterocyclyl, optionally substituted aryl, or optionally substituted heteroaryl;
R⁴ is hydrogen or optionally substituted alkyl;
R^{5a} and R^{5b} are each hydrogen; or R^{5a} and R^{5b} are joined together with their intervening atom to form optionally substituted carbocyclyl; and
R¹¹ is optionally substituted aryl or optionally substituted heteroaryl.

39. The compound of claim 38, or a pharmaceutically acceptable salt thereof, wherein R¹¹ is optionally substituted phenyl, optionally substituted pyrazolyl, optionally substituted imidazolyl, optionally substituted triazolyl, optionally substituted benzimidazolyl, optionally substituted isoxazolyl, optionally substituted oxadiazolyl, optionally substituted thiazolyl, optionally substituted benzothiazolyl, or optionally substituted thiadiazolyl.

40. The compound of any one of claims 38 or 39, or a pharmaceutically acceptable salt thereof, wherein R¹¹ is

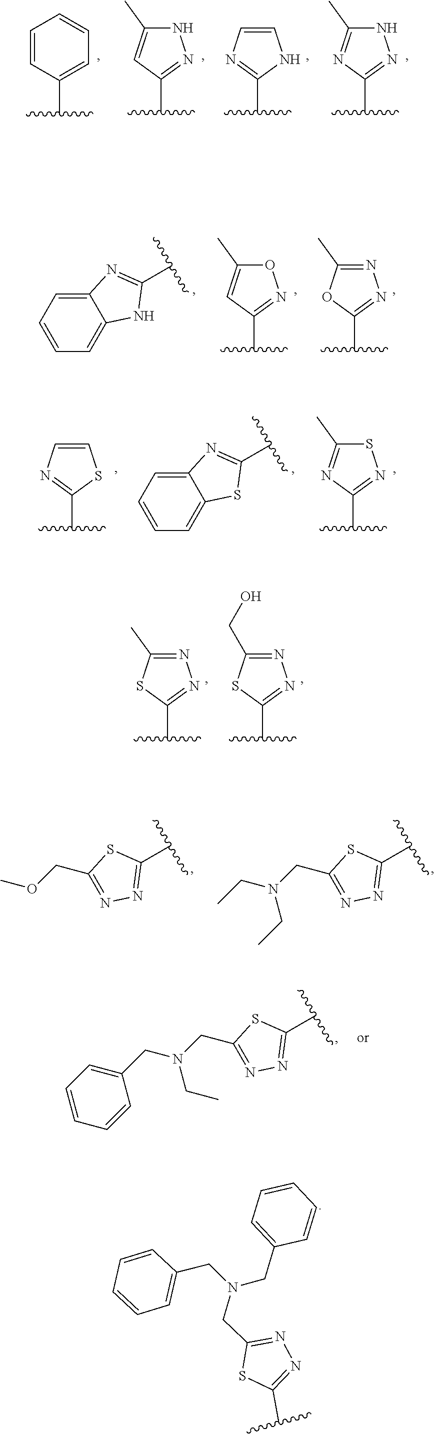

41. The compound of any one of claims 1-40, or a pharmaceutically acceptable salt thereof, wherein R³ is optionally substituted aryl.

42. The compound of any one of claims 1-41, or a pharmaceutically acceptable salt thereof, wherein R³ is phenyl optionally substituted with halogen, optionally substituted C₁₋₃ alkyl, optionally substituted C₂₋₃ alkynyl, —CN, —NO₂, —OR^{1a}, —N(R^{1a})₂, —NR^{1a}C(═O)R^{1a}, optionally substituted C₃₋₄ carbocyclyl, optionally substituted C₃₋₄ heterocyclyl, optionally substituted phenyl, optionally substituted 5-6 membered heteroaryl containing 1, 2, 3, or 4 ring heteroatoms selected from N, O, and S, and/or optionally substituted 8-10 membered heteroaryl containing 1, 2, 3, or 4 ring heteroatoms selected from N, O, and S.

43. The compound of any one of claims 1-42, or a pharmaceutically acceptable salt thereof, wherein R³ is

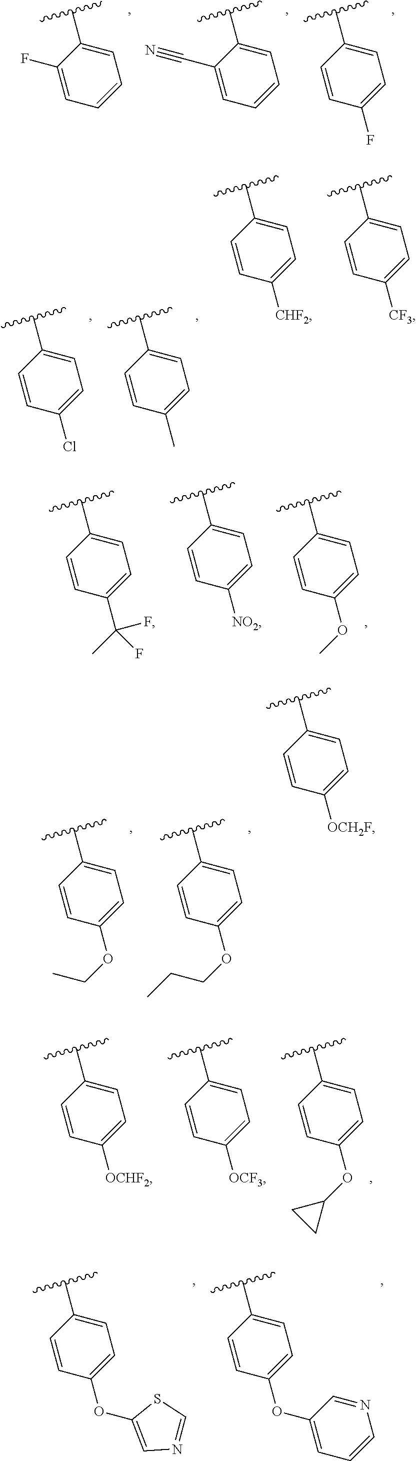

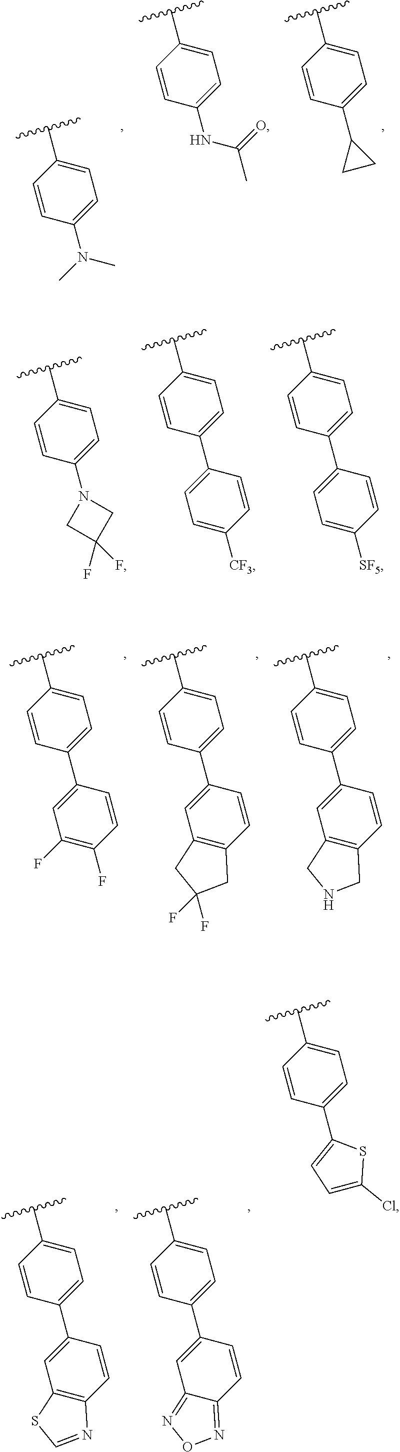

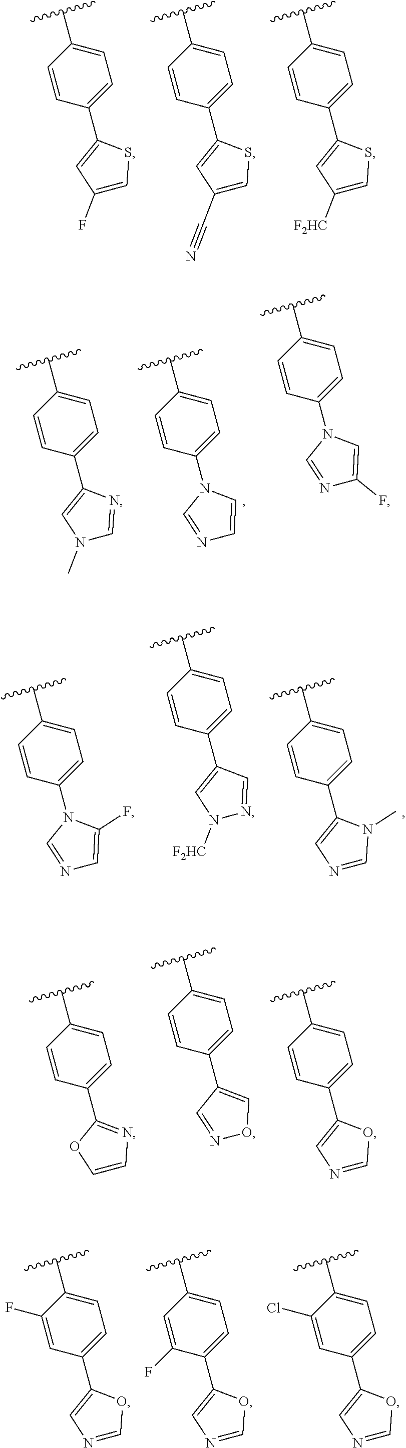

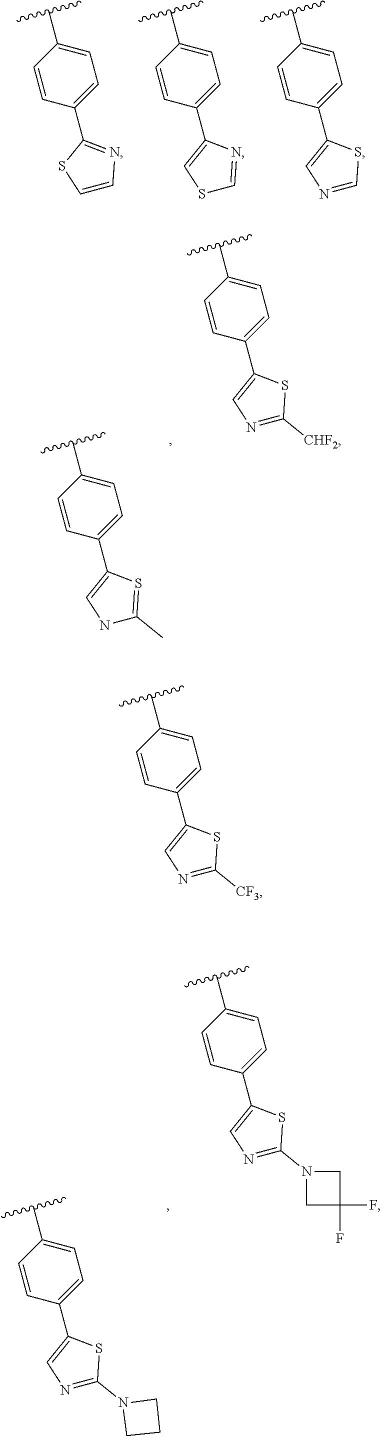

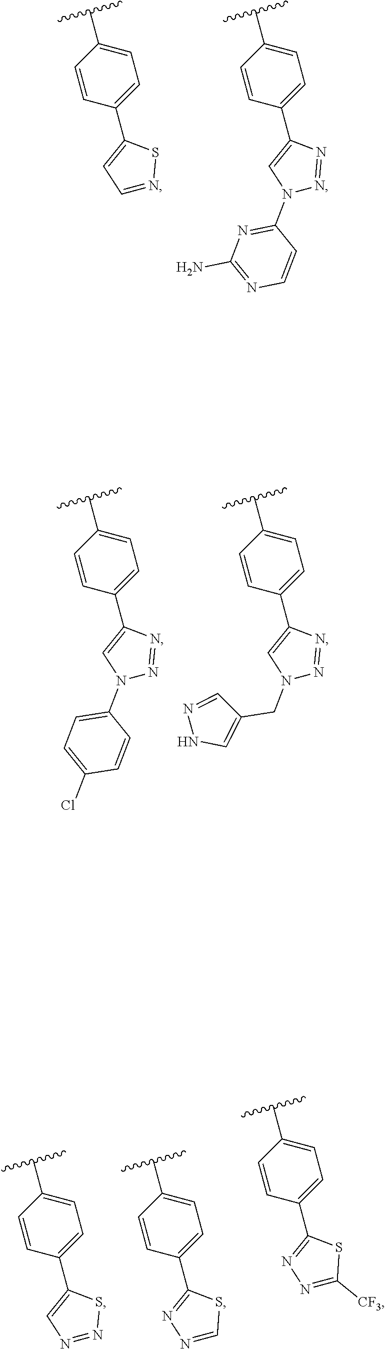

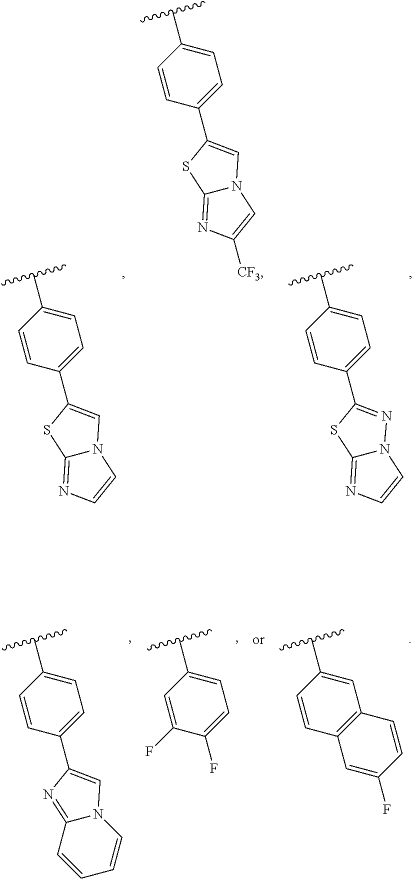 [SMILES: *c1ccc(-c2cn3cc(C(F)(F)F)nc3s2)cc1 ; *c1ccc(-c2cn3ccnc3s2)cc1 ; *c1ccc(-c2nn3ccnc3s2)cc1 ; *c1ccc(-c2cn3ccccc3n2)cc1 ; *c1ccc(F)c(F)c1 ; *c1ccc2cc(F)ccc2c1]

44. The compound of any one of claims 1, 2, 4-37 or 41, wherein the compound is of Formula (III-a-1): 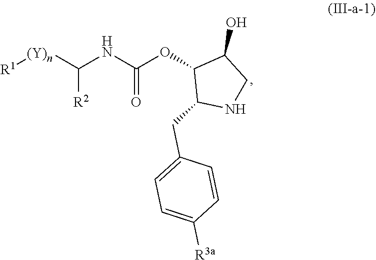
or a pharmaceutically acceptable salt thereof, wherein:
R^{3a} is halogen, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted carbocyclyl, substituted or unsubstituted heterocyclyl, substituted or unsubstituted aryl, substituted or unsubstituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NRA)OR^{A}, —OC(═NRA)SR^{A}, —OC(═NRA)N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each instance of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} are joined together with their intervening atom to form an optionally substituted heterocyclic ring or optionally substituted heteroaryl ring.

45. The compound of claim 44, wherein the compound is of Formula (III-c-1) or (III-c-2): 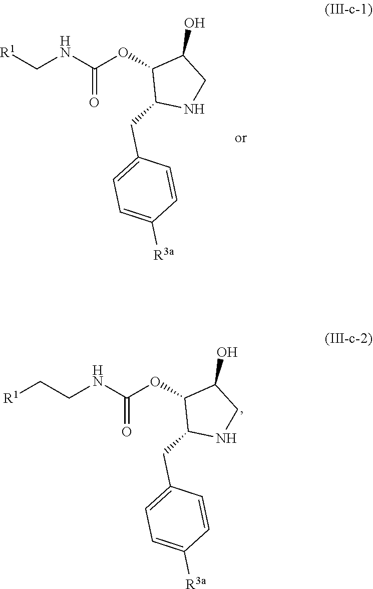
or a pharmaceutically acceptable salt thereof.

46. The compound of any one of claims 44 or 45, wherein R^{3a} is halogen, optionally substituted C₁₋₃ alkyl, optionally substituted C₂₋₃ alkynyl, —CN, —NO₂, —OR^{1a}, —N(R^{1a})₂, —NR^{1a}C(═O)R^{1a}, optionally substituted C₃₋₄ carbocyclyl, optionally substituted C₃₋₄ heterocyclyl, optionally substituted phenyl, optionally substituted 5-6 membered heteroaryl containing 1, 2, 3, or 4 ring heteroatoms selected from N, O, and S, or optionally substituted 8-10 membered heteroaryl containing 1, 2, 3, or 4 ring heteroatoms selected from N, O, and S.

47. The compound of any one of claims 1-46, wherein R⁴ is hydrogen or methyl.

48. The compound of any one of claims 1, 2, 4-37, 41-43, or 47, wherein the compound is of Formula (I-b-1): 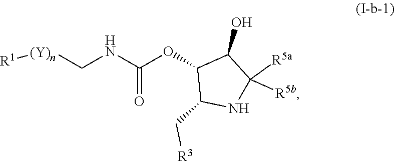
or a pharmaceutically acceptable salt thereof.

49. The compound of any one of claims 1-48, wherein R^{5a} and R^{5b} are each hydrogen, or R^{5a} and R^{5b} are joined together with their intervening atom to form optionally substituted cyclopropyl.

50. The compound of any one of claims 1, 2, 4-37, 41-43, 47, or 49, wherein the compound is of Formula (I-b-3): 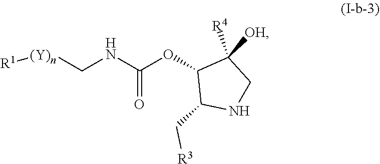
or a pharmaceutically acceptable salt thereof.

51. The compound of any one of claims 1-37 or 41-50, wherein the compound is selected from those in Table 1A and Table 1B, and pharmaceutically acceptable salts thereof.

52. The compound of any one of claims 38-40, wherein the compound is selected from those in Table 2, and pharmaceutically acceptable salts thereof.

53. A composition comprising the compound of any one of claims 1-52, or a pharmaceutically acceptable salt thereof, or a pharmaceutically acceptable salt thereof, and an excipient.

54. A method of modulating protein synthesis in a subject in need thereof or in a cell, tissue, or biological sample, comprising administering to the subject in need thereof or contacting the cell, tissue, or biological sample with an effective amount of:
a compound of any one of claims 1-52, or a pharmaceutically acceptable salt thereof;
or the composition of claim 53.

55. The method of claim 54, wherein modulating protein synthesis comprises modulating synthesis of a target protein.

56. The method of claim 55, wherein modulating protein synthesis comprises decreasing protein synthesis.

57. A method of decreasing protein synthesis in a subject in need thereof or in a cell, tissue, or biological sample, comprising administering to the subject in need thereof or contacting the cell, tissue, or biological sample with an effective amount of:
a compound of any one of claims 1-52, or a pharmaceutically acceptable salt thereof;
or the composition of claim 53.

58. The method of claim 57, wherein decreasing protein synthesis comprises decreasing synthesis of a target protein.

59. The method of any one of claims 55, 56, or 58, further comprising decreasing an amount of mRNA, wherein the mRNA is associated with synthesis of the target protein.

60. The method of any one of claims 55, 56, 58, or 59, wherein the target protein is B-cell lymphoma 2 (BCL-2), MYC proto-oncogene bHLH transcription factor (MYC), cyclin D1 (CCND1), myeloid cell leukemia 1 (MCL-1), anaplastic lymphoma kinase (ALK), or GTPase KRas G12D mutant (KRAS-G12D)

61. A method of treating or preventing a disease in a subject in need thereof, comprising administering to the subject in need thereof an effective amount of:
the compound of any one of claims 1-52, or a pharmaceutically acceptable salt or prodrug thereof;
or the composition of claim 53.

62. The compound of any one of claims 1-52, or a pharmaceutically acceptable salt or prodrug thereof, or the composition of claim 53, for use in treating or preventing a disease in a subject in need thereof.

63. The compound of any one of claims 1-52, or a pharmaceutically acceptable salt or prodrug thereof, or the composition of claim 53, for use in the manufacture of a medicament for treatment or prevention of a disease in a subject in need thereof.

64. The method or compound for use of any one of claims 61-63, wherein the disease is associated with B-cell lymphoma 2 (BCL-2), MYC proto-oncogene bHLH transcription factor (MYC), cyclin D1 (CCND1), myeloid cell leukemia 1 (MCL-1), anaplastic lymphoma kinase (ALK), or GTPase KRas G12D mutant (KRAS-G12D).

65. The method or compound for use of any one of claims 61-64, wherein the disease is a proliferative disease.

66. The method or compound for use of claim 65, wherein the proliferative disease is cancer.

67. The method or compound for use of claim 66, wherein the cancer is prostate cancer, pancreatic cancer, lung cancer, breast cancer, colorectal cancer, endometrial cancer, ovarian cancer, cervical cancer, esophageal cancer, bladder cancer, biliary cancer, hematopoietic cancer, or neuroblastoma.

68. The method or compound for use of any one of claims 61-64, wherein the disease is a neurological disease.

69. The method or compound for use of claim 68, wherein the neurological disease is cerebellar ataxia or a neurodegenerative disease.

70. The method or compound for use of any one of claims 61-64, wherein the disease is an immune disorder.

71. The method or compound for use of claim 70, wherein the immune disorder is psoriasis, lupus, or rheumatoid arthritis.

72. A kit comprising:
the compound of any one of claims 1-52, or a pharmaceutically acceptable salt or prodrug thereof, or the composition of claim 53;
and instructions for its use.
